SEQUENCE LISTING (298 sequences)

SEQ ID NO: 1 - protein, 332 aa, Escherichia coli
MNKKVLTLSAIMASMLFGAAAHAADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSKK

SEQ ID NO: 2 - protein, 360 aa, Thermoanaerobacterium thermosaccharolyticum
MKKILTYLVLVVLVLSALLTGCGNSNTTSSNSSSSSSQQSDKTASSDSGKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFCNNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQ

SEQ ID NO: 3 - protein, 332 aa, Salmonella enterica
MNKKVLTLSAVMASLLFGAHAHAADTRIGVTIYKYDDNFMSVVRKAIEKDGKSAPDVQLLMNDSQNDQSKQNDQIDVLLAKGVKALAINLVDPAAAGTVIEKARGQNVPVVFFNKEPSRKALDSYDKAYYVGTDSKESGVIQGDLIAKHWQANQGWDLNKDGKIQYVLLKGEPGHPDAEARTTYVVKELNDKGIQTEQLALDTAMWDTAQAKDKMDAWLSGPNANKIEVVIANNDAMAMGAVEALKAHNKSSIPVFGVDALPEALALVKSGAMAGTVLNDANNQAKATFDLAKNLAEGKGAADGTSWKIENKIVRVPYVGVDKDNLSEFTQK

SEQ ID NO: 4 - protein, 356 aa, Caldicellulosiruptor hydrothermalis
MFNKKKFWVVLVSMVLIISLVLVGCGKKSTNDSSNGTSEENKPYIGVAIYKFDDTFMTGVRNAIAKEGEGKAKLDFVDCQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHNGIMEYVMITGEPGHQDAILRTEYSIKAVEAAGIKTKALAQDTAMWDRVKGQEKMQAFLASFGDRIEAVFCNNDDMALGAIEALKAAGYFKNGKYIPVVGVDATTPGLQALEEGTLLGTVLNDAKAQGKATFNLAYVLAKGEKPTKENVGFDITDGKYIWVPYQKVTKDNLEEMKKYVNEQ

SEQ ID NO: 5 - protein, 356 aa, Caldicellulosiruptor obsidiansis
MLNKKKFWVVLVSIVLAISLVLVGCGKKSTNENSGGTSEDNKPYIGVAIYKFDDTFMTGVRNAIAKEGEGKAKLDFVDCQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHDGIMQYVMITGEPGHQDAILRTEYSIKAVEAAGIRVKCLAQDTAMWDRVKGQEKMQAFLASFGDKIEAVFCNNDDMALGAIEALKAAGYFKDGKYVPVVGVDATTPGLQALEEGTLLGTVLNDAKAQGKATFNLAYVLAKGEKPTKENVGFEITDGKYIWVPYQKVTKDNLEEMKKYVNEQ

SEQ ID NO: 6 - protein, 350 aa, Paenibacillus sp
MKKKWLFVLMAGMMLTTAACNNGGSSSTGSDSTGGDAVGGSTPQVGVAIYKFDDTFMTGVRNAMSDAANGVAKLDIVDSQNAQPTQNEKIDLFISKKYSSMIINPVDRTAAGVIIDKAKTANTPVVFLNREPIAEDMNKWDKVYYVGAKAEESGTISGQLIVDYWKANPKADKNGDGKLQYVLLQGEPGHQDAELRTKFSVQAIQDAGIEVEALAVDTAMWDRVKGQEKMQTFLASHGDKIEAVLANNDDMALGAIEALKAAGYFSGDKYMPVVGVDATAPAVQALEDGTLLGTVLNDAKSQGKASVAIAAALSKGEAPNKENTGFDITDGKYVWIAYKKITKDNIADAK

SEQ ID NO: 7 - protein, 354 aa, Clostridium saccharolyticum
MKRLRKGIFLFFFIVWTAFPLYGCAPMEQKKDVGESATSEAGTEGVPEETGPKIGVSIYRYDDTFMKLYRQELKQYLEETYHAEVIMRNAGGDQKEQDKQVNQFISDGCDGIIVNPVEIPAAQELADACSRAGIPLVFINREPKEEEQKRWREKQMAVSCVGTDSRQAGTYQGEIILETLNKGDFNGDGVVSYVMLMGEKGNEDSQYRTEYSIKALEEGGMKTEELFSGNGNWNKDEGKKLAKQALASWGNRIEVFFCNNDSMANGALEAVEEAGRIPGKDIYLVGVDALQDTVTYIKEGRMTGTVLNDHEGQSQMAADTLKKMIDGESVETRYQVDYIKVTAISTFQTLKGED

SEQ ID NO: 8 - protein, 354 aa, Butyrivibrio proteoclasticus
MKKKMICYMIIAALALSLMAGCSNTQESEPVQESVAYSSYSDAKVGVCIYQKSDNFMSLFSSELVKYLVSRGFSKDNIILYDSNNDENVQLSQVEELIASGINALIINPVNSSVAHSITDMASASNIPLVYINREPSGDEENRWEMYQLNVCYVGCDARQSGIYQGEILLSLGKNKLDHNGDGKIQYFMIEGAPENIDAGYRTLYSVSALQNSEMEMDCLLDEVGNWDETTASLLVSKGIQNGLKPEVIICNNDAMALGAIKAAEKSGLVPGEDVYIVGVDALPEAIEMIKAGKLAGTVYNDYVLQSHKSADAVINYLKGIDNEHYIGCDYVKVDIDNAESIAGLTNTDEEDID

SEQ ID NO: 9 - protein, 383 aa, Roseburia intestinalis
MKRKVVSVILATAMVASMVAGCGGSNNASTNNAGTTTDAAASDASSDTSNDAAATEAADAGDAAADAATDADASLADKKVGVCIYQFSDNFMTLFRTELENYLVEKGFSKDNITIVDGANDQATQTGQIDNFITEGVDVLIINPVNSSSAATITDKVVAAGIPLVYINREPDEEEQKRWSDNNWDVTYVGCDARQSGTFQGEMISDLGLDTVDLNGNGKIDYVMVEGDPENVDAQYRTEYSVKALEDAGLEVNCLSDQVGNWQQDQAQQIVANALGQYGNDVEVVFCNNDAMALGALQAIQSAGRTVGTDIYLVGVDALSEALEDVLAGTMTGTVFNDHFSQSHSAADAAINYITGAGNDHYIGCDYVKVTKDNAQDVLDMVK

SEQ ID NO: 10 - protein, 350 aa, Faecalibacterium prausnitzii
MKMISRRDFLKASAVVGATAAMTACGGSSSTSTAASSVASSTAASAAATNGSANIGVCIYQFADNFMTLYRADLEGYLKDMGYSVTIMDGKNDQNTQTEQINTFLQQGVDVLVINPVQTTSAQTIVDTVSPSGTPIVFINREPEESVLDSYKGKCCYVGADARQSGTYQGELILATDTQGDINGDGKITYIMCKGDPENIDAQYRTEYSIKALTDAGKEVECLYEYLDNWDQTTAQQDVANALSQYGEKIEVVFCNNDAMALGALQSIQQAGRTVGKDVYLVGVDALVEAVQNVVDGNMTGTVLNDDVGQATKAAEATKLFVEGKDVEKYYWVDYVKVTKDNASQYLKED

SEQ ID NO: 11 - protein, 338 aa, Clostridium ljungdahlii
MIVKKCMKSIAVTGLLTIILGTGCSNSLSSNKNEPVIGFVAYEFNNTWITELKNEIYKVSSGKARVDIWNGDNIQTVENDKINLFINRKVNVLDINPVDVNAAGQIIEKCKKANIPTVFVNRQPKKEDMEKWNKVYYVGAKAEQSGTIQGQMLVNYFKGHPTQDGTIRYIMLKGETRNQDAEKRTQYSIKALKDSGFKVQKVAEDTAMWDRTKAQEKMTSFISSYGPNFDCVIANNDDMALGAVDALKAAGYFNGGKYVPVVGVDATAPAVKAVEDGTLFGTVLNDAAKQGDAAFDLSYILSKGKIPDESNFKYKVTDGKYIWIDYKMITKENVQDAK

SEQ ID NO: 12 - protein, 336 aa, Clostridium autoethanogenum
MKKCMKSIAVTGLLTIILGTGCSNSLSSNKNEPVIGFVAYEFNNTWITELKNEMYKVSNGKARVDIWNGNNIQTVENDKISLFINRKVDVLDINPVDVNAAGQIIEKCKKANIPTVFVNRQPKKEDVEKWNKVYYVGAKAEQSGTIQGQMLVNYFKGHPTQDGTIRYIMLKGEMRNQDAEKRTQYSIKALEDSGFKVQKVAEDTAMWDRTKAQEKMTSFISSYGPNFDCVIANNDDMALGAVDALKAAGYFNGGKYVPVVGVDATAPAVKAVEDGTLFGTVLNDAAKQGDAAFDLSYILSKGKIPDESNFKYKITDGKYIWIDYKMITKENVQDAK

SEQ ID NO: 13 - protein, 367 aa, Roseburia intestinalis
MCGAEKVRHMLMGEQVLKKWKKSKKMTVAFGGILVMSVVIGGCGGREDAKKSIKIGISVYDQYDTFVSEMMKDFNDYATKKEEETGVAINIDTYNASASQSTQNSQVENMITEGCDVICVNLVDRTDPTAIIDLAEKNNIPVIFFNRELVEEDLERWTRLYYVGAQAFESGIMQGELAAEAFLTDQSLDKNGDGIFQYVVLEGEAGHQDAIVRTEYSVSTMIDSGVEVEKLGYAIANWNRAQAQTKMAQLMSQFGDSIELVIANNDDMALGAIDALKASGLTKDEWPAVIGIDGTDVGLEAVKNKEMIGTVYNDKEGQADAMLNLAYELSTGSDLSDLNLIDGKYIRLPYARVTCDDVDSYMEGDTE

SEQ ID NO: 14 - protein, 360 aa, Erysipelothrix rhusiopathiae
MKKLSKLILVSLLALTLFGCSSKGGAEGNANEGGKTYNIGVAIYKFDDNFMTLYREELASYFKEVGEKDGNTYKLDIQDGKQDQANQTEQINNFIAQGKDLIIANMVDPTAAGSIINSAKAKEIPVVFINREPETQELEIWPGKTTYVGADATQSGTIQGYMIANLENKGDIDGDGSVSYITLMGDPANVDAKQRTEYSVKGLEEKGVKTNALAQPYQANWDTAKGQEFTANALEQFGNKLEVVFANNDGMAVGAVTAIEAAGRKVGEDIFVVGVDAIPDAIELLKGGKLTGTVLNDHFNQSHTAVDVALELLQGKDVSAYYWHDYVGVTKPEEAELKRAEARKETVEEAVKRYAERDAQ

SEQ ID NO: 15 - protein, 338 aa, Eubacterium rectale
MMILCFALILSFVSCSNTRVDEKKQIYIGVTCYDQKDTFIGELIETFKKECASLDTDKYDISMTIMDAAGSQRAQDDQVQEMIEDGCNVLCINLADRTDLSHIINAAMEKDIPIIFFNREPVDEDLNRWDKLYYVGAKAKQSGQMQGELIADYIKNNPGVDKNGDGRIQYVILEGEMGHQDAIVRTESVTESMKNNGLQIEKLSCQIANWNRAQAQNRMTQLIGQYKNSIELVIANNDAMALGAIDAYEKLGVTESNVPAFFGVDGTDDGLEAVQQSKLAATVYNDKEGQAMAMAQLAYLAATGGSMKNIKFEDKKYVYLPYEKVTPDNVNEFVKDEQ

SEQ ID NO: 16 - protein, 310 aa, synthetic construct
  REGION 1..310: note = ecGGBP (with signal peptide replaced with M)
MADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNQADGKGAADGTNWKIDNKVVRVPYVGVDKDNLAEFSKK

SEQ ID NO: 17 - protein, 309 aa, synthetic construct
  REGION 1..309: note = ecGGBP (with signal sequence removed)
ADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNQADGKGAADGTNWKIDNKVVRVPYVGVDKDNLAEFSKK

SEQ ID NO: 18 - protein, 312 aa, synthetic construct
  REGION 1..312: note = ttGGBP (with signal peptide replaced with M)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFCNNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQ

SEQ ID NO: 19 - protein, 310 aa, synthetic construct
  REGION 1..310: note = stGGBP (with signal peptide replaced with M)
MADTRIGVTIYKYDDNFMSVVRKAIEKDGKSAPDVQLLMNDSQNDQSKQNDQIDVLLAKGVKALAINLVDPAAAGTVIEKARGQNVPVVFFNKEPSRKALDSYDKAYYVGTDSKESGVIQGDLIAKHWQANQGWDLNKDGKIQYVLLKGEPGHPDAEARTTYVVKELNDKGIQTEQLALDTAMWDTAQAKDKMDAWLSGPNANKIEVVIANNDAMAMGAVEALKAHNKSSIPVFGVDALPEALALVKSGAMAGTVLNDANNQAKATFDLAKNLAEGKGAADGTSWKIENKIVRVPYVGVDKDNLSEFTQK

SEQ ID NO: 20 - protein, 316 aa, synthetic construct
  REGION 1..316: note = chyGGBP (with signal peptide replaced with M)
MKPYIGVAIYKFDDTFMTGVRNAIAKEGEGKAKLDFVDCQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHNGIMEYVMITGEPGHQDAILRTEYSIKAVEAAGIKTKALAQDTAMWDRVKGQEKMQAFLASFGDRIEAVFCNNDDMALGAIEALKAAGYFKNGKYIPVVGVDATTPGLQALEEGTLLGTVLNDAKAQGKATFNLAYVLAKGEKPTKENVGFDITDGKYIWVPYQKVTKDNLEEMKKYVNEQ

SEQ ID NO: 21 - protein, 316 aa, synthetic construct
  REGION 1..316: note = cobGGBP (with signal peptide replaced with M)
MKPYIGVAIYKFDDTFMTGVRNAIAKEGEGKAKLDFVDCQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHDGIMQYVMITGEPGHQDAILRTEYSIKAVEAAGIRVKCLAQDTAMWDRVKGQEKMQAFLASFGDKIEAVFCNNDDMALGAIEALKAAGYFKDGKYVPVVGVDATTPGLQALEEGTLLGTVLNDAKAQGKATFNLAYVLAKGEKPTKENVGFEITDGKYIWVPYQKVTKDNLEEMKKYVNEQ

SEQ ID NO: 22 - protein, 307 aa, synthetic construct
  REGION 1..307: note = pspGGBP (with signal peptide replaced with M)
MVGVAIYKFDDTFMTGVRNAMSDAANGVAKLDIVDSQNAQPTQNEKIDLFISKKYSSMIINPVDRTAAGVIIDKAKTANTPVVFLNREPIAEDMNKWDKVYYVGAKAEESGTISGQLIVDYWKANPKADKNGDGKLQYVLLQGEPGHQDAELRTKFSVQAIQDAGIEVEALAVDTAMWDRVKGQEKMQTFLASHGDKIEAVLANNDDMALGAIEALKAAGYFSGDKYMPVVGVDATAPAVQALEDGTLLGTVLNDAKSQGKASVAIAAALSKGEAPNKENTGFDITDGKYVWIAYKKITKDNIADAK

SEQ ID NO: 23 - protein, 307 aa, synthetic construct
  REGION 1..307: note = csaGGBP (with signal peptide replaced with M)
METGPKIGVSIYRYDDTFMKLYRQELKQYLEETYHAEVIMRNAGGDQKEQDKQVNQFISDGCDGIIVNPVEIPAAQELADACSRAGIPLVFINREPKEEEQKRWREKQMAVSCVGTDSRQAGTYQGEIILETLNKGDFNGDGVVSYVMLMGEKGNEDSQYRTEYSIKALEEGGMKTEELFSGNGNWNKDEGKKLAKQALASWGNRIEVFFCNNDSMANGALEAVEEAGRIPGKDIYLVGVDALQDTVTYIKEGRMTGTVLNDHEGQSQMAADTLKKMIDGESVETRYQVDYIKVTAISTFQTLKGED

SEQ ID NO: 24 - protein, 314 aa, synthetic construct
  REGION 1..314: note = bprGGBP (with signal peptide replaced with M)
MDAKVGVCIYQKSDNFMSLFSSELVKYLVSRGFSKDNIILYDSNNDENVQLSQVEELIASGINALIINPVNSSVAHSITDMASASNIPLVYINREPSGDEENRWEMYQLNVCYVGCDARQSGIYQGEILLSLGKNKLDHNGDGKIQYFMIEGAPENIDAGYRTLYSVSALQNSEMEMDCLLDEVGNWDETTASLLVSKGIQNGLKPEVIICNNDAMALGAIKAAEKSGLVPGEDVYIVGVDALPEAIEMIKAGKLAGTVYNDYVLQSHKSADAVINYLKGIDNEHYIGCDYVKVDIDNAESIAGLTNTDEEDID

SEQ ID NO: 25 - protein, 306 aa, synthetic construct
  REGION 1..306: note = rinGGBP_A (with signal peptide replaced with M)
MKVGVCIYQFSDNFMTLFRTELENYLVEKGFSKDNITIVDGANDQATQTGQIDNFITEGVDVLIINPVNSSSAATITDKVVAAGIPLVYINREPDEEEQKRWSDNNWDVTYVGCDARQSGTFQGEMISDLGLDTVDLNGNGKIDYVMVEGDPENVDAQYRTEYSVKALEDAGLEVNCLSDQVGNWQQDQAQQIVANALGQYGNDVEVVFCNNDAMALGALQAIQSAGRTVGTDIYLVGVDALSEALEDVLAGTMTGTVFNDHFSQSHSAADAAINYITGAGNDHYIGCDYVKVTKDNAQDVLDMVK

SEQ ID NO: 26 - protein, 300 aa, synthetic construct
  REGION 1..300: note = fprGGBP (with signal peptide replaced with M)
MSANIGVCIYQFADNFMTLYRADLEGYLKDMGYSVTIMDGKNDQNTQTEQINTFLQQGVDVLVINPVQTTSAQTIVDTVSPSGTPIVFINREPEESVLDSYKGKCCYVGADARQSGTYQGELILATDTQGDINGDGKITYIMCKGDPENIDAQYRTEYSIKALTDAGKEVECLYEYLDNWDQTTAQQDVANALSQYGEKIEVVFCNNDAMALGALQSIQQAGRTVGKDVYLVGVDALVEAVQNVVDGNMTGTVLNDDVGQATKAAEATKLFVEGKDVEKYYWVDYVKVTKDNASQYLKED

SEQ ID NO: 27 - protein, 305 aa, synthetic construct
  REGION 1..305: note = cljGGBP (with signal peptide replaced with M)
MPVIGFVAYEFNNTWITELKNEIYKVSSGKARVDIWNGDNIQTVENDKINLFINRKVNVLDINPVDVNAAGQIIEKCKKANIPTVFVNRQPKKEDMEKWNKVYYVGAKAEQSGTIQGQMLVNYFKGHPTQDGTIRYIMLKGETRNQDAEKRTQYSIKALKDSGFKVQKVAEDTAMWDRTKAQEKMTSFISSYGPNFDCVIANNDDMALGAVDALKAAGYFNGGKYVPVVGVDATAPAVKAVEDGTLFGTVLNDAAKQGDAAFDLSYILSKGKIPDESNFKYKVTDGKYIWIDYKMITKENVQDAK

SEQ ID NO: 28 - protein, 306 aa, synthetic construct
  REGION 1..306: note = cauGGBP (with signal peptide replaced with M)
MEPVIGFVAYEFNNTWITELKNEMYKVSNGKARVDIWNGNNIQTVENDKISLFINRKVDVLDINPVDVNAAGQIIEKCKKANIPTVFVNRQPKKEDVEKWNKVYYVGAKAEQSGTIQGQMLVNYFKGHPTQDGTIRYIMLKGEMRNQDAEKRTQYSIKALEDSGFKVQKVAEDTAMWDRTKAQEKMTSFISSYGPNFDCVIANNDDMALGAVDALKAAGYFNGGKYVPVVGVDATAPAVKAVEDGTLFGTVLNDAAKQGDAAFDLSYILSKGKIPDESNFKYKITDGKYIWIDYKMITKENVQDAK

SEQ ID NO: 29 - protein, 318 aa, synthetic construct
  REGION 1..318: note = rinGGBP_B (with signal peptide replaced with M)
MKSIKIGISVYDQYDTFVSEMMKDFNDYATKKEEETGVAINIDTYNASASQSTQNSQVENMITEGCDVICVNLVDRTDPTAIIDLAEKNNIPVIFFNRELVEEDLERWTRLYYVGAQAFESGIMQGELAAEAFLTDQSLDKNGDGIFQYVVLEGEAGHQDAIVRTEYSVSTMIDSGVEVEKLGYAIANWNRAQAQTKMAQLMSQFGDSIELVIANNDDMALGAIDALKASGLTKDEWPAVIGIDGTDVGLEAVKNKEMIGTVYNDKEGQADAMLNLAYELSTGSDLSDLNLIDGKYIRLPYARVTCDDVDSYMEGDTE

SEQ ID NO: 30 - protein, 327 aa, synthetic construct
  REGION 1..327: note = erhGGBP (with signal peptide replaced with M)
MKTYNIGVAIYKFDDNFMTLYREELASYFKEVGEKDGNTYKLDIQDGKQDQANQTEQINNFIAQGKDLIIANMVDPTAAGSIINSAKAKEIPVVFINREPETQELEIWPGKTTYVGADATQSGTIQGYMIANLENKGDIDGDGSVSYITLMGDPANVDAKQRTEYSVKGLEEKGVKTNALAQPYQANWDTAKGQEFTANALEQFGNKLEVVFANNDGMAVGAVTAIEAAGRKVGEDIFVVGVDAIPDAIELLKGGKLTGTVLNDHFNQSHTAVDVALELLQGKDVSAYYWHDYVGVTKPEEAELKRAEARKETVEEAVKRYAERDAQ

SEQ ID NO: 31 - protein, 316 aa, synthetic construct
  REGION 1..316: note = ereGGBP (with signal peptide replaced with M)
MKQIYIGVTCYDQKDTFIGELIETFKKECASLDTDKYDISMTIMDAAGSQRAQDDQVQEMIEDGCNVLCINLADRTDLSHIINAAMEKDIPIIFFNREPVDEDLNRWDKLYYVGAKAKQSGQMQGELIADYIKNNPGVDKNGDGRIQYVILEGEMGHQDAIVRTESVTESMKNNGLQIEKLSCQIANWNRAQAQNRMTQLIGQYKNSIELVIANNDAMALGAIDAYEKLGVTESNVPAFFGVDGTDDGLEAVQQSKLAATVYNDKEGQAMAMAQLAYLAATGGSMKNIKFEDKKYVYLPYEKVTPDNVNEFVKDEQ

SEQ ID NO: 32 - protein, 319 aa, synthetic construct
  REGION 1..319: note = ecGGBPW183C (with signal peptide replaced with M; a W183C mutation; and a GGSHHHHHH at C-terminus)
MADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMCDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSKKGGSHHHHHH

SEQ ID NO: 33 - protein, 319 aa, synthetic construct
  REGION 1..319: note = ecGGBP (with signal peptide replaced with M and a GGSHHHHHH at C-terminus)
MADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNQADGKGAADGTNWKIDNKVVRVPYVGVDKDNLAEFSKKGGSHHHHHH

SEQ ID NO: 34 - protein, 316 aa, synthetic construct
  REGION 1..316: note = csaGGBP (with signal peptide replaced with M and a GGSHHHHHH at C-terminus)
METGPKIGVSIYRYDDTFMKLYRQELKQYLEETYHAEVIMRNAGGDQKEQDKQVNQFISDGCDGIIVNPVEIPAAQELADACSRAGIPLVFINREPKEEEQKRWREKQMAVSCVGTDSRQAGTYQGEIILETLNKGDFNGDGVVSYVMLMGEKGNEDSQYRTEYSIKALEEGGMKTEELFSGNGNWNKDEGKKLAKQALASWGNRIEVFFCNNDSMANGALEAVEEAGRIPGKDIYLVGVDALQDTVTYIKEGRMTGTVLNDHEGQSQMAADTLKKMIDGESVETRYQVDYIKVTAISTFQTLKGEDGGSHHHHHH

SEQ ID NO: 35 - protein, 323 aa, synthetic construct
  REGION 1..323: note = bprGGBP (with signal peptide replaced with M and a GGSHHHHHH at C-terminus)
MDAKVGVCIYQKSDNFMSLFSSELVKYLVSRGFSKDNIILYDSNNDENVQLSQVEELIASGINALIINPVNSSVAHSITDMASASNIPLVYINREPSGDEENRWEMYQLNVCYVGCDARQSGIYQGEILLSLGKNKLDHNGDGKIQYFMIEGAPENIDAGYRTLYSVSALQNSEMEMDCLLDEVGNWDETTASLLVSKGIQNGLKPEVIICNNDAMALGAIKAAEKSGLVPGEDVYIVGVDALPEAIEMIKAGKLAGTVYNDYVLQSHKSADAVINYLKGIDNEHYIGCDYVKVDIDNAESIAGLTNTDEEDIDGGSHHHHHH

SEQ ID NO: 36 - protein, 315 aa, synthetic construct
  REGION 1..315: note = rinGGBP_A (with signal peptide replaced with M and a GGSHHHHHH at C-terminus)
MKVGVCIYQFSDNFMTLFRTELENYLVEKGFSKDNITIVDGANDQATQTGQIDNFITEGVDVLIINPVNSSSAATITDKVVAAGIPLVYINREPDEEEQKRWSDNNWDVTYVGCDARQSGTFQGEMISDLGLDTVDLNGNGKIDYVMVEGDPENVDAQYRTEYSVKALEDAGLEVNCLSDQVGNWQQDQAQQIVANALGQYGNDVEVVFCNNDAMALGALQAIQSAGRTVGTDIYLVGVDALSEALEDVLAGTMTGTVFNDHFSQSHSAADAAINYITGAGNDHYIGCDYVKVTKDNAQDVLDMVKGGSHHHHHH

SEQ ID NO: 37 - protein, 309 aa, synthetic construct
  REGION 1..309: note = fprGGBP (with signal peptide replaced with M and a GGSHHHHHH at C-terminus)
MSANIGVCIYQFADNFMTLYRADLEGYLKDMGYSVTIMDGKNDQNTQTEQINTFLQQGVDVLVINPVQTTSAQTIVDTVSPSGTPIVFINREPEESVLDSYKGKCCYVGADARQSGTYQGELILATDTQGDINGDGKITYIMCKGDPENIDAQYRTEYSIKALTDAGKEVECLYEYLDNWDQTTAQQDVANALSQYGEKIEVVFCNNDAMALGALQSIQQAGRTVGKDVYLVGVDALVEAVQNVVDGNMTGTVLNDDVGQATKAAEATKLFVEGKDVEKYYWVDYVKVTKDNASQYLKEDGGSHHHHHH

SEQ ID NO: 38 - protein, 314 aa, synthetic construct
  REGION 1..314: note = cljGGBP (with signal peptide replaced with M and a GGSHHHHHH at C-terminus)
MPVIGFVAYEFNNTWITELKNEIYKVSSGKARVDIWNGDNIQTVENDKINLFINRKVNVLDINPVDVNAAGQIIEKCKKANIPTVFVNRQPKKEDMEKWNKVYYVGAKAEQSGTIQGQMLVNYFKGHPTQDGTIRYIMLKGETRNQDAEKRTQYSIKALKDSGFKVQKVAEDTAMWDRTKAQEKMTSFISSYGPNFDCVIANNDDMALGAVDALKAAGYFNGGKYVPVVGVDATAPAVKAVEDGTLFGTVLNDAAKQGDAAFDLSYILSKGKIPDESNFKYKVTDGKYIWIDYKMITKENVQDAKGGSHHHHHH

SEQ ID NO: 39 - protein, 315 aa, synthetic construct
  REGION 1..315: note = cauGGBP (with signal peptide replaced with M and a GGSHHHHHH at C-terminus)
MEPVIGFVAYEFNNTWITELKNEMYKVSNGKARVDIWNGNNIQTVENDKISLFINRKVDVLDINPVDVNAAGQIIEKCKKANIPTVFVNRQPKKEDVEKWNKVYYVGAKAEQSGTIQGQMLVNYFKGHPTQDGTIRYIMLKGEMRNQDAEKRTQYSIKALEDSGFKVQKVAEDTAMWDRTKAQEKMTSFISSYGPNFDCVIANNDDMALGAVDALKAAGYFNGGKYVPVVGVDATAPAVKAVEDGTLFGTVLNDAAKQGDAAFDLSYILSKGKIPDESNFKYKITDGKYIWIDYKMITKENVQDAKGGSHHHHHH

SEQ ID NO: 40 - protein, 327 aa, synthetic construct
  REGION 1..327: note = rinGGBP_B (with signal peptide replaced with M and a GGSHHHHHH at C-terminus)
MKSIKIGISVYDQYDTFVSEMMKDFNDYATKKEEETGVAINIDTYNASASQSTQNSQVENMITEGCDVICVNLVDRTDPTAIIDLAEKNNIPVIFFNRELVEEDLERWTRLYYVGAQAFESGIMQGELAAEAFLTDQSLDKNGDGIFQYVVLEGEAGHQDAIVRTEYSVSTMIDSGVEVEKLGYAIANWNRAQAQTKMAQLMSQFGDSIELVIANNDDMALGAIDALKASGLTKDEWPAVIGIDGTDVGLEAVKNKEMIGTVYNDKEGQADAMLNLAYELSTGSDLSDLNLIDGKYIRLPYARVTCDDVDSYMEGDTEGGSHHHHHH

SEQ ID NO: 41 - protein, 336 aa, synthetic construct
  REGION 1..336: note = erhGGBP (with signal peptide replaced with M and a GGSHHHHHH at C-terminus)
MKTYNIGVAIYKFDDNFMTLYREELASYFKEVGEKDGNTYKLDIQDGKQDQANQTEQINNFIAQGKDLIIANMVDPTAAGSIINSAKAKEIPVVFINREPETQELEIWPGKTTYVGADATQSGTIQGYMIANLENKGDIDGDGSVSYITLMGDPANVDAKQRTEYSVKGLEEKGVKTNALAQPYQANWDTAKGQEFTANALEQFGNKLEVVFANNDGMAVGAVTAIEAAGRKVGEDIFVVGVDAIPDAIELLKGGKLTGTVLNDHFNQSHTAVDVALELLQGKDVSAYYWHDYVGVTKPEEAELKRAEARKETVEEAVKRYAERDAQGGSHHHHHH

SEQ ID NO: 42 - protein, 325 aa, synthetic construct
  REGION 1..325: note = ereGGBP (with signal peptide replaced with M and a GGSHHHHHH at C-terminus)
MKQIYIGVTCYDQKDTFIGELIETFKKECASLDTDKYDISMTIMDAAGSQRAQDDQVQEMIEDGCNVLCINLADRTDLSHIINAAMEKDIPIIFFNREPVDEDLNRWDKLYYVGAKAKQSGQMQGELIADYIKNNPGVDKNGDGRIQYVILEGEMGHQDAIVRTESVTESMKNNGLQIEKLSCQIANWNRAQAQNRMTQLIGQYKNSIELVIANNDAMALGAIDAYEKLGVTESNVPAFFGVDGTDDGLEAVQQSKLAATVYNDKEGQAMAMAQLAYLAATGGSMKNIKFEDKKYVYLPYEKVTPDNVNEFVKDEQGGSHHHHHH

SEQ ID NO: 43 - protein, 321 aa, synthetic construct
  REGION 1..321: note = ttGGBP (with signal peptide replaced with M and a GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFCNNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 44 - protein, 325 aa, synthetic construct
  REGION 1..325: note = cobGGBP (with signal peptide replaced with M and a GGSHHHHHH at C-terminus)
MKPYIGVAIYKFDDTFMTGVRNAIAKEGEGKAKLDFVDCQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHDGIMQYVMITGEPGHQDAILRTEYSIKAVEAAGIRVKCLAQDTAMWDRVKGQEKMQAFLASFGDKIEAVFCNNDDMALGAIEALKAAGYFKDGKYVPVVGVDATTPGLQALEEGTLLGTVLNDAKAQGKATFNLAYVLAKGEKPTKENVGFEITDGKYIWVPYQKVTKDNLEEMKKYVNEQGGSHHHHHH

SEQ ID NO: 45 - protein, 325 aa, synthetic construct
  REGION 1..325: note = chyGGBP (with signal peptide replaced with M and a GGSHHHHHH at C-terminus)
MKPYIGVAIYKFDDTFMTGVRNAIAKEGEGKAKLDFVDCQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHNGIMEYVMITGEPGHQDAILRTEYSIKAVEAAGIKTKALAQDTAMWDRVKGQEKMQAFLASFGDRIEAVFCNNDDMALGAIEALKAAGYFKNGKYIPVVGVDATTPGLQALEEGTLLGTVLNDAKAQGKATFNLAYVLAKGEKPTKENVGFDITDGKYIWVPYQKVTKDNLEEMKKYVNEQGGSHHHHHH

SEQ ID NO: 46 - protein, 316 aa, synthetic construct
  REGION 1..316: note = pspGGBP (with signal peptide replaced with M and a GGSHHHHHH at C-terminus)
MVGVAIYKFDDTFMTGVRNAMSDAANGVAKLDIVDSQNAQPTQNEKIDLFISKKYSSMIINPVDRTAAGVIIDKAKTANTPVVFLNREPIAEDMNKWDKVYYVGAKAEESGTISGQLIVDYWKANPKADKNGDGKLQYVLLQGEPGHQDAELRTKFSVQAIQDAGIEVEALAVDTAMWDRVKGQEKMQTFLASHGDKIEAVLANNDDMALGAIEALKAAGYFSGDKYMPVVGVDATAPAVQALEDGTLLGTVLNDAKSQGKASVAIAAALSKGEAPNKENTGFDITDGKYVWIAYKKITKDNIADAKGGSHHHHHH

SEQ ID NO: 47 - protein, 325 aa, synthetic construct
  REGION 1..325: note = cobGGBP.W181C (Signaling cys mutant of cobGGBP with signal peptide replaced with M and GGSHHHHHH at C-terminus)
MKPYIGVAIYKFDDTFMTGVRNAIAKEGEGKAKLDFVDAQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHDGIMQYVMITGEPGHQDAILRTEYSIKAVEAAGIRVKSLAQDTAMCDRVKGQEKMQAFLASFGDKIEAVFANNDDMALGAIEALKAAGYFKDGKYVPVVGVDATTPGLQALEEGTLLGTVLNDAKAQGKATFNLAYVLAKGEKPTKENVGFEITDGKYIWVPYQKVTKDNLEEMKKYVNEQGGSHHHHHH

SEQ ID NO: 48 - protein, 325 aa, synthetic construct
  REGION 1..325: note = chyGGBP.W181C (Signaling cys mutants of chyGGBP with signal peptide replaced with M and GGSHHHHHH at C-terminus)
MKPYIGVAIYKFDDTFMTGVRNAIAKEGEGKAKLDFVDAQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHNGIMEYVMITGEPGHQDAILRTEYSIKAVEAAGIKTKALAQDTAMCDRVKGQEKMQAFLASFGDRIEAVFANNDDMALGAIEALKAAGYFKNGKYIPVVGVDATTPGLQALEEGTLLGTVLNDAKAQGKATFNLAYVLAKGEKPTKENVGFDITDGKYIWVPYQKVTKDNLEEMKKYVNEQGGSHHHHHH

SEQ ID NO: 49 - protein, 321 aa, synthetic construct
  REGION 1..321: note = ttGGBP11C (ttGGBP cys mutant with signal peptide replaced with M and GGSHHHHHH at C-terminus)
MKQLNIGVAICKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 50 - protein, 321 aa, synthetic construct
  REGION 1..321: note = ttGGBP16C (ttGGBP cys mutant with signal peptide replaced with M and GGSHHHHHH at C-terminus)
MKQLNIGVAIYKFDDCFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 51 - protein, 321 aa, synthetic construct
  REGION 1..321: note = ttGGBP17C (ttGGBP cys mutant with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 52 - protein, 321 aa, synthetic construct
  REGION 1..321: note = ttGGBP42C (ttGGBP cys mutant with signal peptide replaced with M and GGSHHHHHH at C-terminus)
MKQLNIGVAIYKFDDTFMTGVRNAMTAEAQGKAKLNMVDSQCSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 53 - protein, 321 aa, synthetic construct
  REGION 1..321: note = ttGGBP67C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPCDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 54 - protein, 321 aa, synthetic construct
  REGION 1..321: note = ttGGBP91C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNCEPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 55 - protein, 321 aa, synthetic construct
  REGION 1..321: note = ttGGBP92C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNRCPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 56 - protein, 321 aa, synthetic construct
  REGION 1..321: note = ttGGBP111C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKCEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 57 - protein, 321 aa, synthetic construct
  REGION 1..321: note = ttGGBP148C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGCPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 58 - protein, 321 aa, synthetic construct
  REGION 1..321: note = ttGGBP151C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGCQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 59 - protein, 321 aa, synthetic construct
  REGION 1..321: note = ttGGBP152C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHCDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 60 - protein, 321 aa, synthetic construct
  REGION 1..321: note = ttGGBP181C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYACWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 61 - protein, 321 aa, synthetic construct
  REGION 1..321: note = ttGGBP182C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 62 - protein, 321 aa, synthetic construct
  REGION 1..321: note = ttGGBP183C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWCRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 63 - protein, 321 aa, synthetic construct
  REGION 1..321: note = ttGGBP257C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVCNDAKNQAKATFNIAYELAQGITPTKDNIGYDITDGKYVWIPYKKITKDNISDAEQGGSHHHHHH

SEQ ID NO: 64 - protein, 321 aa, synthetic construct
  REGION 1..321: note = ttGGBP259C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNCAKNQAKATFNIAYELAQGITPTKDNIGYDITDGKYVWIPYKKITKDNISDAEQGGSHHHHHH

SEQ ID NO: 65 - protein, 321 aa, synthetic construct
  REGION 1..321: note = ttGGBP300C (ttGGBP cys mutant with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYCKITKDNISDAEQGGSHHHHHH

SEQ ID NO: 66 - protein, 321 aa, synthetic construct
  REGION 1..321: note = ttGGBP17C.1 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 67 - protein, 321 aa, synthetic construct
  REGION 1..321: note = ttGGBP17C.2 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDPTAAGTIIDKAKQANIPVVFFNREPLPEDMKKWDKVYYVGAKAEQSGILQGQIMADYWKAHPEADKNHDGVMQYVMLMGQPGHPDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 68 - protein, 321 aa, synthetic construct
  REGION 1..321: note = ttGGBP17C.3 (affinity-tuning mutant, 17C background (Table 6) with signal peptide replaced with M and GGSHHHHHH at C-terminus)
MKQLNIGVAIYKFDDNCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDAMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 69 - protein, 321 aa, synthetic construct
  REGION 1..321: note = ttGGBP17C.4 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGQQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 70 - protein, 321 aa, synthetic construct
  REGION 1..321: note = ttGGBP17C.5 (affinity-tuning mutant, 17C background (Table 6) with signal peptide replaced with M and GGSHHHHHH at C-terminus)
MKQLNIGVAIYKFDATCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 71 - protein, 321 aa, synthetic construct
  REGION 1..321: note = ttGGBP17C.6 (affinity-tuning mutant, 17C background (Table 6) with signal peptide replaced with M and GGSHHHHHH at C-terminus)
MKQLNIGVAIYKFDETCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 72 - protein, 321 aa, synthetic construct
  REGION 1..321: note = ttGGBP17C.7 (affinity-tuning mutant, 17C background (Table 6) with signal peptide replaced with M and GGSHHHHHH at C-terminus)
MKQLNIGVAIYKFDNTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 73 - protein, 321 aa, synthetic construct
  REGION 1..321: note = ttGGBP17C.8 (affinity-tuning mutant, 17C background (Table 6) with signal peptide replaced with M and GGSHHHHHH at C-terminus)
MKQLNIGVAIYKFDDNCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 74 - protein, 321 aa, synthetic construct
  REGION 1..321: note = ttGGBP17C.9 (affinity-tuning mutant, 17C background (Table 6) with signal peptide replaced with M and GGSHHHHHH at C-terminus)
MKQLNIGVAIYKFDDSCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 75 - protein, 321 aa, synthetic construct
  REGION 1..321: note = ttGGBP17C.10 (affinity-tuning mutant, 17C background (Table 6) with signal peptide replaced with M and GGSHHHHHH at C-terminus)
MKQLNIGVAIYKFDDTCMTAVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 76 - protein, 321 aa, synthetic construct
  REGION 1..321: note = ttGGBP17C.11 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDAAAPGIQAIKDGTLLGTVLNDAKNQAKATFNIAYELAQGITPTKDNIGYDITDGKYVWIPYKKITKDNISDAEQGGSHHHHHH

SEQ ID NO: 77 - protein, 321 aa, synthetic construct
  REGION 1..321: note = ttGGBP17C.19 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLDDAKNQAKATFNIAYELAQGITPTKDNIGYDITDGKYVWIPYKKITKDNISDAEQGGSHHHHHH

SEQ ID NO: 78 - protein, 321 aa, synthetic construct
  REGION 1..321: note = ttGGBP17C.20 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLSDAKNQAKATFNIAYELAQGITPTKDNIGYDITDGKYVWIPYKKITKDNISDAEQGGSHHHHHH

SEQ ID NO: 79 - protein, 321 aa, synthetic construct
  REGION 1..321: note = ttGGBP17C.21 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLADAKNQAKATFNIAYELAQGITPTKDNIGYDITDGKYVWIPYKKITKDNISDAEQGGSHHHHHH

SEQ ID NO: 80 - protein, 321 aa, synthetic construct
  REGION 1..321: note = ttGGBP17C.22 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDNKNQAKATFNIAYELAQGITPTKDNIGYDITDGKYVWIPYKKITKDNISDAEQGGSHHHHHH

SEQ ID NO: 81 - protein, 321 aa, synthetic construct
  REGION 1..321: note = ttGGBP17C.23 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDQKNQAKATFNIAYELAQGITPTKDNIGYDITDGKYVWIPYKKITKDNISDAEQGGSHHHHHH

SEQ ID NO: 82 - protein, 321 aa, synthetic construct
  REGION 1..321: note = ttGGBP17C.24 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDRKNQAKATFNIAYELAQGITPTKDNIGYDITDGKYVWIPYKKITKDNISDAEQGGSHHHHHH

SEQ ID NO: 83 - protein, 321 aa, synthetic construct
  REGION 1..321: note = ttGGBP17C.25 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDKKNQAKATFNIAYELAQGITPTKDNIGYDITDGKYVWIPYKKITKDNISDAEQGGSHHHHHH

SEQ ID NO: 84 - protein, 321 aa, synthetic construct
  REGION 1..321: note = ttGGBP17C.26 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDWKNQAKATFNIAYELAQGITPTKDNIGYDITDGKYVWIPYKKITKDNISDAEQGGSHHHHHH

SEQ ID NO: 85 - protein, 321 aa, synthetic construct
  REGION 1..321: note = ttGGBP17C.27 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDFKNQAKATFNIAYELAQGITPTKDNIGYDITDGKYVWIPYKKITKDNISDAEQGGSHHHHHH

SEQ ID NO: 86 - protein, 321 aa, synthetic construct
  REGION 1..321: note = ttGGBP17C.28 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDYKNQAKATFNIAYELAQGITPTKDNIGYDITDGKYVWIPYKKITKDNISDAEQGGSHHHHHH

SEQ ID NO: 87 - protein, 321 aa, synthetic construct
  REGION 1..321: note = ttGGBP17C.29 (affinity-tuning mutant, 17C background (Table 6) with signal peptide replaced with M and GGSHHHHHH at C-terminus)
MKQLNIGVAIYKFDDTCMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANWDRVTAHDKMAAWLSSFGDKIEAVFANNDDMALGAIEALKSAGYFTGNKYIPVVGVDATAPGIQAIKDGTLLGTVLNDSKNQAKATFNIAYELAQGITPTKDNIGYDITDGKYVWIPYKKITKDNISDAEQGGSHHHHHH

SEQ ID NO: 88 - protein, 321 aa, synthetic construct
  REGION 1..321: note = ttGGBP182C.2.0 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 89 - protein, 321 aa, synthetic construct
  REGION 1..321: note = ttGGBP182C.2.1 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDSILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 90 - protein, 321 aa, synthetic construct
  REGION 1..321: note = ttGGBP182C.2.3 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDNILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 91 - protein, 321 aa, synthetic construct
  REGION 1..321: note = ttGGBP182C.2.4 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDMILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 92 - protein, 321 aa, synthetic construct
  REGION 1..321: note = ttGGBP182C.2.5 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGQQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 93 - protein, 321 aa, synthetic construct
  REGION 1..321: note = ttGGBP182C.2.6 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGNQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 94 - protein, 321 aa, synthetic construct
  REGION 1..321: note = ttGGBP182C.2.7 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGFQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 95 - protein, 321 aa, synthetic construct
  REGION 1..321: note = ttGGBP182C.2.8 (affinity-tuning mutant, 182C background (Table 6) with signal peptide replaced with M and GGSHHHHHH at C-terminus)
MKQLNIGVAIYKFDNTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 96 - protein, 321 aa, synthetic construct
  REGION 1..321: note = ttGGBP182C.2.9 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDFILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 97 - protein, 321 aa, synthetic construct
  REGION 1..321: note = ttGGBP182C.3 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGQPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 98 - protein, 321 aa, synthetic construct
  REGION 1..321: note = ttGGBP182C.4 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 99 - protein, 321 aa, synthetic construct
  REGION 1..321: note = ttGGBP182C.5 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDPTAAGTIIDKAKQANIPVVFFNREPLPEDMKKWDKVYYVGAKAEQSGILQGQIMADYWKAHPEADKNHDGVMQYVMLMGQPGHPDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 100 - protein, 321 aa, synthetic construct
  REGION 1..321: note = ttGGBP182C.6 (affinity-tuning mutant, 182C background (Table 6) with signal peptide replaced with M and GGSHHHHHH at C-terminus)
MKQLNIGVAIYKFDDNFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANCDRVTAHDKMAAWLSSFGDKIEAVFANNDAMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 101 - protein, 321 aa, synthetic construct
  REGION 1..321: note = ttGGBP182C.7 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGSPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 102 - protein, 321 aa, synthetic construct
  REGION 1..321: note = ttGGBP182C.8 (affinity-tuning mutant, 182C background (Table 6) with signal peptide replaced with M and GGSHHHHHH at C-terminus)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGKPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 103 - protein, 321 aa, synthetic construct
  REGION 1..321: note = ttGGBP182C.9 (affinity-tuning mutant, 182C background (Table 6) with signal peptide replaced with M and GGSHHHHHH at C-terminus)
MKQLNIGVAIYKFDNTFMTGVRNAMTAEAQGKAKLNMVDSQNSQPTQNDQVDLFITKKMNALAINPVDRTAAGTIIDKAKQANIPVVFFNREPLPEDMKKWDKVYYVGAKAEQSGILQGQIMADYWKAHPEADKNHDGVMQYVMLMGQPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 104 - protein, 9 aa, synthetic construct
  REGION 1..9: note = GGSHHHHHH Tag Sequence
GGSHHHHHH

SEQ ID NO: 105 - DNA, 1328 bp, synthetic construct
  misc_feature 1..1328: note = csaGGBP Exemplary Expression Construct (Table 2)
  source 1..1328: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatggaaacaggtccaaaaattggtgtaagtatatacaggtacgacgacaccttcatgaagctgtatcgccaggaactgaagcagtacctggaagaaacctaccatgcggaagtgatcatgcgcaatgctggtggtgaccagaaagagcaggacaaacaggtgaaccagttcatctcagacggatgtgacgggatcatagtgaatccggtggaaattccggcagctcaggagcttgcggatgcgtgcagtcgtgcgggaatcccccttgtgttcataaaccgtgaacccaaggaagaagaacagaaacgttggcgcgaaaagcagatggcagtttcgtgtgtaggcaccgatagccgtcaggcgggtacctatcagggcgaaatcatcctggaaaccctgaacaaaggcgacttcaacggtgatggtgtcgtgtcctacgtgatgctcatgggtgagaagggcaatgaggactcgcaataccggacggaatacagcatcaaagcgctggaagaaggcggcatgaaaaccgaagagctgttttcgggcaacggcaactggaacaaagacgaaggcaagaaactggcgaaacaggcgcttgcctcttggggtaaccgcatagaggtgttcttctgcaacaacgattcgatggcgaatggtgcgttggaagcagtggaggaggcaggtaggatcccaggcaaagacatctacctggttggtgtggatgcgttgcaggatacggtgacctacatcaaagaaggccgtatgaccggaacagtcctgaacgaccacgaaggtcagagccagatggcagctgatacgctgaagaaaatgatcgatggagagtcagtggagactcggtatcaggtggactacatcaaagtgactgccatctctacgtttcaaactttaaaaggtgaagatggaggaagccatcatcatcatcatcattaataatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 106 - DNA, 1349 bp, synthetic construct
  misc_feature 1..1349: note = bprGGBP Exemplary Expression Construct (Table 2)
  source 1..1349: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatggatgcaaaagttggtgtatgtatatatcaaaaatcggacaacttcatgtcgctcttctccagcgaactggtgaagtacctggtgagtcgtgggttcagcaaggacaacatcatcctgtacgactccaacaacgacgagaacgtgcagttgtcgcaggtggaagagctcattgcgtctgggatcaatgcgctcatcatcaacccagtgaactcgagtgttgctcactcgatcacggacatggcatctgcctcgaacatcccgttagtctacatcaaccgtgaaccgtctggtgacgaagagaaccgttgggagatgtatcagctgaacgtgtgctatgtcggctgtgatgcacgccaatctggcatctaccagggggaaatcctcctgtctctgggcaaaaacaagctggatcacaacggagatggcaagatccagtacttcatgatcgaaggtgcaccggagaacatcgatgcaggctatcgtacgctgtactccgtttctgctctgcagaacagcgaaatggagatggattgcctgctggatgaagtgggcaactgggatgaaaccactgccagtctgctggtctccaagggcatccagaatgggctcaaaccggaggtcatcatctgcaacaacgacgcgatggcactgggtgccatcaaagcggcggaaaaatcgggtctggttccgggtgaagacgtctacatcgtgggtgttgacgccttgcctgaagcgatcgaaatgatcaaagcagggaagctcgcaggtaccgtgtacaacgactacgtgctgcagtcccacaaaagcgcagatgcggtcatcaactacctgaaagggatcgacaacgagcactacatcggttgcgattacgtgaaagtggatatcgacaacgcggaaagcattgcggggttgacaaatacagatgaagaagatattgatggaggaagccatcatcatcatcatcattagtaataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 107 - DNA, 1325 bp, synthetic construct
  misc_feature 1..1325: note = rinGGBP_A Exemplary Expression Construct (Table 2)
  source 1..1325: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaagttggtgtatgtatttaccaatttagtgacaacttcatgacgctcttccgtaccgaactggaaaactacctggtggaaaaaggctttagcaaggacaacatcaccatcgttgatggcgctaacgatcaggctacgcagactggccagatcgacaacttcatcaccgaaggggtggatgtcctcatcatcaatccggtcaacagcagcagtgcagcgacgattaccgataaagtggtggcagcaggcattccgctggtctacatcaaccgagaaccggatgaagaagagcagaaacgctggagtgacaacaactgggacgtgacgtacgtgggttgcgatgcgcgtcagtctgggacattccagggtgagatgatcagcgatcttggcctggatacggtcgatctgaacggaaacgggaaaatcgactacgtcatggtggaaggtgatccagagaacgtggatgcccagtatcgcacggaatactccgtgaaagcactggaagatgcgggtctcgaagtgaactgcttgagcgatcaggttgggaactggcagcaggatcaggcacagcagattgtggcgaatgctctcggtcagtatggcaacgacgtagaggttgtgttctgcaacaacgacgctatggcgttaggtgccttacaggcgattcagagcgcaggtcgtaccgttggtactgacatctacctggttggtgtggatgcgttgagcgaagcactggaggatgtgctggctggtaccatgaccggaactgtgttcaacgaccacttcagtcagtcccatagtgcggcagatgcggctatcaactacatcacaggagctggaaacgaccactacatcggctgcgattacgtgaaagtgaccaaagacaacgcacaagatgtgttagatatggttaaaggaggaagccatcatcatcatcatcattaatgataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 108 - DNA, 1307 bp, synthetic construct
  misc_feature 1..1307: note = fprGGBP Exemplary Expression Construct (Table 2)
  source 1..1307: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgtctgctaatattggagtttgtatttatcaatttgccgacaacttcatgaccctctatcgtgctgacctggaaggctacctgaaggacatgggctattccgtcaccatcatggacgggaaaaacgaccagaacacccaaaccgagcagatcaacaccttcctgcagcaaggcgttgacgtgctggtcatcaacccagttcagaccactagtgcacagaccatcgtagacaccgtttctccgtctggtaccccaatcgtgttcatcaaccgtgaacctgaggaaagcgtcctggatagctacaagggcaagtgctgctacgtgggagctgatgctcggcagagtggtacgtaccagggtgagctgatcctggcaaccgatacgcaaggcgacatcaacggtgatggcaagatcacctacatcatgtgcaaaggcgatccggagaacatcgacgcacagtaccgtaccgagtacagcatcaaggcactgaccgatgcaggcaaggaagtggagtgcctgtacgagtacctggacaactgggatcagaccactgcacagcaagacgtggctaacgcactgtcccagtatggcgagaagatcgaagtcgtcttctgcaacaacgacgctatggcactgggcgcattgcagagcatccagcaagctggtcgtaccgttggcaaagacgtctacctggttggagtcgatgcgctggtagaggctgtccagaacgttgtcgacggtaacatgaccggaaccgtgctcaacgacgacgttggtcaggcaacgaaagctgcagaagcgaccaagctgttcgtggagggcaaagacgtggagaagtactactgggtggactacgtcaaagtcaccaaggataatgcaagtcaatatctgaaagaagatggaggaagccatcatcatcatcatcattagtaataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 109 - DNA, 1322 bp, synthetic construct
  misc_feature 1..1322: note = cljGGBP Exemplary Expression Construct (Table 2)
  source 1..1322: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgccagttattggatttgttgcttatgaatttaacaacacgtggatcaccgaactgaagaacgagatctacaaggtgtctagcggcaaagcgcgtgtggacatctggaacggcgataacatccagaccgttgagaacgacaagatcaacctcttcatcaaccgcaaagtgaacgtgctcgacatcaacccggttgatgtcaacgcagctggacagatcatcgaaaagtgcaaaaaagcgaacattccgaccgtgttcgtgaatcgccaaccgaaaaaagaggatatggagaaatggaacaaagtgtactacgttggtgccaaagccgaacagtcgggtaccatccaagggcagatgctcgtgaactacttcaaaggccatccgactcaggatggcactatccgctacatcatgctcaagggtgaaacgcgaaaccaggatgcggagaaacgcacccagtacagcatcaaagcgctgaaagacagcggtttcaaggtgcagaaggttgcggaagacaccgcaatgtgggatcgtacgaaggcacaggagaagatgacttcgttcatatcctcctacggacccaacttcgactgtgtgatagcgaacaacgacgacatggccttaggagccgttgatgccctcaaagcggcaggctacttcaacggcggcaaatatgtgccggtggttggtgtggatgccactgctccggctgtcaaagcagtggaagacggaacgttgttcggaactgtgctgaacgatgctgcgaaacagggtgatgcggcctttgatctgtcgtacatcctttccaaagggaagatcccggatgaaagcaacttcaagtacaaggtgacggatggcaagtacatttggatcgactacaagatgatcactaaagaaaatgttcaagatgcaaaaggaggaagccatcatcatcatcatcattagtgataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 110 - DNA, 1325 bp, synthetic construct
  misc_feature 1..1325: note = cauGGBP Exemplary Expression Construct (Table 2)
  source 1..1325: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatggaaccagtaattggttttgttgcttatgagttcaacaacacgtggattaccgaactgaagaacgagatgtacaaggtgtctaacggcaaagcgcgtgtggacatctggaacggcaacaacatccagacggtcgagaacgacaagatctccctgttcatcaaccgcaaagtcgacgttctggacatcaacccggttgacgtgaacgcagctggacagatcatcgagaaatgcaaaaaagcgaacattccgacggtgttcgtgaaccgtcaaccgaaaaaggaagatgtggaaaagtggaacaaagtctactacgttggtgcgaaagccgaacagtctggcaccatacagggacagatgctggtgaactacttcaaagggcatccgacccaggatggaaccatccgctacatcatgctcaaaggggaaatgcgcaaccaggatgcggagaaacgcacccagtacagcatcaaagccctggaagattctgggttcaaggtccagaaggtggcagaagacaccgctatgtgggatcgtaccaaggctcaggagaagatgacttcgttcatctcctcatacggaccgaacttcgactgtgtgatcgccaacaacgacgacatggccttaggagctgttgatgccctgaaagctgcaggctacttcaacggcggcaaatatgtgccggtggttggtgtggatgctactgctccggcagtgaaagctgtcgaggatgggacactgttcggaactgtcctgaacgatgcagcgaaacagggtgatgcggcctttgatctgtcgtacatcctttccaaggggaagattccggatgaaagcaacttcaagtacaagatcacggatggcaagtacatttggatcgactacaagatgattactaaagaaaatgttcaagatgctaaaggaggaagccatcatcatcatcatcattagtaataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 111 - DNA, 1361 bp, synthetic construct
  misc_feature 1..1361: note = rinGGBP_B Exemplary Expression Construct (Table 2)
  source 1..1361: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaatcaataaaaattggtataagtgtttatgatcagtatgacacctttgtgagcgaaatgatgaaagacttcaacgattatgcgaccaagaaagaagaagagactggtgttgccatcaacatcgacacatacaacgctagtgcaagccagtctacgcagaacagtcaggtcgagaacatgatcacggaaggctgcgatgtgatctgtgtcaacctggttgacaggacggatccaactgcgatcatcgatcttgcggagaaaaacaacatccccgtgatttttttcaaccgtgaactggtggaggaagacctggaacgttggacaaggctgtactacgttggtgcacaggcgttcgaaagcggcatcatgcagggtgaactggctgcagaagcctttctgaccgatcagagcctggacaagaacggagatgggatcttccagtacgtggtgcttgaaggtgaggctggacaccaggatgccatcgtgcgtacggagtactcggttagcaccatgatcgatagcggagtggaagtggagaaactgggctacgcaatcgccaactggaatcgtgcacaggctcagacgaaaatggcgcagctgatgtcccagtttggtgacagcatagagctggtcatcgccaacaacgacgacatggctctaggagccatagatgcgctcaaggcgtctggtctgaccaaggacgaatggccggcagtcattgggatcgatggaacggatgtgggcttagaggcggtcaagaacaaagagatgatcggtaccgtgtacaacgataaggaaggccaagcggatgcgatgctgaacctggcgtacgaactgagtaccggtagcgatctgtcggatctgaacctgatcgatggcaaatacatccgtctgccttatgcgcgagtgacgtgtgatgatgtggatagttatatggaaggtgataccgaaggaggaagccatcatcatcatcatcattaataatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 112 - DNA, 1388 bp, synthetic construct
  misc_feature 1..1388: note = erhGGBP Exemplary Expression Construct (Table 2)
  source 1..1388: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaaacttataatattggtgttgcaatttataagttcgatgacaacttcatgacgctgtatcgtgaagaactggcgagctacttcaaagaagtgggtgagaaagacggcaacacgtacaaactggacatccaggatggcaagcaggaccaagctaaccagaccgaacagatcaacaactttatcgcacagggtaaagacctgatcatcgcgaacatggtcgatccgaccgcagcgggtagcatcatcaacagcgcgaaagcgaaagaaatcccggtggtcttcatcaaccgagaaccggaaacccaggaactggaaatctggccgggcaaaaccacctacgttggtgcggatgcgacccagtcaggcaccattcagggctacatgatcgcgaacttggagaacaaaggcgacatcgacggtgatgggagtgtcagctacatcacgctgatgggagatcctgcgaacgtggatgcgaaacagcgtaccgaatacagcgtgaaaggcctggaagagaaaggcgtgaaaaccaacgcgcttgcgcagccgtaccaagcgaactgggataccgcgaaaggccaggaattcaccgcaaacgcgctggaacagtttggcaacaaactggaagtggtgttcgcgaacaacgatggcatggcggttggtgcggtgaccgcgattgaagctgcgggacgcaaagtcggagaggacatcttcgtggttggtgtcgatgccattccggatgccatcgagctcctgaaaggcggtaaactgaccggtactgtcctcaacgaccacttcaaccagagccataccgcggtggatgtggcactggaactgctgcagggcaaagatgtgagcgcctactactggcatgactacgttggcgtgaccaaaccggaagaagcggaactgaaacgtgcagaagcacgcaaagagaccgtggaagaagcggttaaacgttatgcagaacgtgatgctcaaggaggaagccatcatcatcatcatcattaataatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 113 - DNA, 1355 bp, synthetic construct
  misc_feature 1..1355: note = ereGGBP Exemplary Expression Construct (Table 2)
  source 1..1355: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacagatttatattggtgtaacttgttatgatcagaaggataccttcattggagagctgatcgagaccttcaagaaggaatgcgcgtctctggataccgacaagtacgacatcagcatgaccatcatggatgctgcaggtagccagcgtgcacaggatgatcaggtgcaggagatgatcgaagacggttgcaacgtactgtgcatcaacctggcagatcgtaccgacttgtcgcacatcatcaacgcggcgatggagaaggatatcccgatcatcttcttcaaccgagaaccggtggatgaggatctcaaccgttgggacaagctgtactacgtaggagcaaaggcgaaacagagcggtcagatgcaaggagagctcattgcggactacatcaaaaacaacccgggtgtggacaagaacggtgatgggaggatccagtacgtcatcctggaaggggaaatgggtcatcaggatgccatcgtacggactgaaagcgtgaccgaatcgatgaagaacaacggtctgcagatcgagaagctgagctgccagatcgcgaactggaatcgagctcaagctcagaaccggatgacgcagctgattggccagtacaagaactcgatcgagctggtgatcgccaacaacgatgccatggctctaggtgcgatcgatgcctacgagaaactcggtgtaacggaaagcaacgttccagcgtttttcggtgtggatggtacagacgatggactggaagcagtgcagcagagcaagcttgcggcaacggtgtacaacgacaaggaaggtcaggcgatggcgatggctcagttggcctatctcgctgcaactggtggaagcatgaagaacatcaaattcgaagacaaaaagtatgtgtatctgccgtatgagaaggtgacaccggataacgttaatgaatttgttaaagatgaacaaggaggaagccatcatcatcatcatcattgataataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 114 - DNA, 1343 bp, synthetic construct
  misc_feature 1..1343: note = ttGGBP Exemplary Expression Construct (Table 2)
  source 1..1343: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaattaaatattggtgttgcgatttataaattcgatgacaccttcatgaccggtgttcggaatgcgatgactgcggaagcgcaaggcaaggcgaagctcaacatggttgacagccagaacagccaaccaacccagaacgatcaggttgacctgttcatcacgaagaagatgaacgcacttgcgatcaacccggtggatcgcactgcagcaggcaccatcatcgacaaagccaaacaggccaacattccggtggtcttcttcaaccgtgaaccgctgccggaagacatgaagaaatgggataaagtgtactatgtgggtgcgaaagcggaacagagtggcattttgcagggtcagatcatggctgactactggaaagcccatcctgaagcggacaagaaccatgatggcgtgatgcagtacgtcatgctgatgggtcagccaggtcatcaggatgcgatactgcgtacacagtacagcatccaaacggtgaaagatgcaggcatcaaagtgcaggaactggccaaagactacgcaaactgggatcgcgttaccgcgcatgacaaaatggcagcgtggttgtcgagctttggcgacaagatcgaagccgtcttctgcaacaacgacgacatggcgttgggtgccatcgaagcgctgaagtctgcaggctacttcaccggcaacaaatacatcccggttgtgggcgtggatgcgaccgcaccgggcattcaggcgatcaaggatggtaccctgttgggaaccgtgttgaacgatgcgaagaaccaggcgaaagccaccttcaacatcgcctacgaactggctcagggcattaccccgacgaaggacaacatagggtacgacatcacagacgggaagtacgtgtggattccgtacaaaaagatcacgaaagacaatatatcagatgctgaacaaggaggaagccatcatcatcatcatcattagtaataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 115 - DNA, 1355 bp, synthetic construct
  misc_feature 1..1355: note = cobGGBP Exemplary Expression Construct (Table 2)
  source 1..1355: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaaccttatataggtgttgctatatataagtttgacgacaccttcatgaccggtgtgcgtaacgcgattgcgaaagaaggcgaaggcaaagcgaaactggatttcgtcgattgccagaacagccagtcgacccagaacgacaagatcgacctgttcatcaccaagaaggtcgatgcactggcgatcaacccagttgatcgcacagcagcaggtgtgctcatcgacaaggcgaaacaggcgaacatccctgttgtcttcttcaaccgtgaaccgcttccggaagacatgaagaaatgggataaggtgtactacgtgggtgcgaaagcggaacagagtggcactctgcaaggcgaaatcatggcggagtactggaaaagccatccggaagcggacaaaaaccatgatggcattatgcagtatgtcatgatcactggtgaacctggacaccaggatgccatactccggactgagtactccataaaggcggtggaagcggctggtatccgcgtgaaatgcctggcgcaggataccgcgatgtgggatcgagtgaaaggccaggaaaagatgcaggcattccttgcgagctttggcgacaaaatcgaagccgtgttctgcaacaacgacgacatggcactgggtgcgattgaagcgctgaaagcggctggctacttcaaggatggcaagtatgtgccggttgtgggcgtggatgcgaccaccccgggtctgcaggcgctggaagaaggtaccctgcttggtaccgtgttgaacgatgcgaaagcgcaaggtaaggctactttcaacctcgcttacgtgctggcgaaaggcgaaaaaccgaccaaagaaaacgtgggtttcgaaatcaccgatggcaaatacatctgggttccgtaccagaaagtgaccaaagacaacctggaagaaatgaaaaaatatgtgaatgaacagggaggaagccatcatcatcatcatcattaataatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 116 - DNA, 1355 bp, synthetic construct
  misc_feature 1..1355: note = chyGGBP Exemplary Expression Construct (Table 2)
  source 1..1355: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaaccttatattggtgttgcaatatataagtttgacgacacattcatgaccggtgttcgcaacgcgattgcgaaagaaggcgaaggcaaagcgaaactggactttgtcgattgccagaacagccagtcgacccagaacgacaagatcgacctcttcataaccaagaaagtggacgctttggcgatcaatccggttgatcgtaccgcggcaggtgtgttgatcgacaaggcgaaacaggcgaacatcccagtcgtcttcttcaaccgagagccgttaccggaagatatgaagaaatgggataaggtgtactacgttggtgccaaagctgaacagtcaggcactctgcagggggaaatcatggcagagtactggaaaagccatccggaagccgacaagaaccacaacggcatcatggaatacgtgatgatcaccggtgaaccgggtcatcaggatgcgatcttgcgtaccgagtactcgatcaaagcggttgaggctgcgggtatcaagaccaaagcgcttgctcaggatacagccatgtgggatagggtgaaaggtcaggagaagatgcaggcgttccttgcgagctttggcgatcggattgaggctgtattctgcaacaacgacgatatggcactgggtgccattgaagcgctcaaagcagcagggtacttcaagaacggcaaatacatccctgttgtgggtgtggatgcgaccactccgggtctgcaggcgctggaagaaggtaccctgctgggtaccgtcttgaacgacgccaaagctcagggtaaggctacgttcaacttggcgtatgtgctggcgaaaggcgaaaaaccgaccaaagagaatgtcggcttcgacatcacggatggcaagtacatttgggtgccgtaccagaaagtgaccaaggacaacttggaagagatgaaaaaatacgtaaatgaacaaggaggaagccatcatcatcatcatcattaataatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 117 - DNA, 1328 bp, synthetic construct
  misc_feature 1..1328: note = pspGGBP23 Exemplary Expression Construct (Table 2)
  source 1..1328: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatggttggtgtagcaatttataagtttgatgataccttcatgacaggcgttcgaaacgcgatgagcgatgcagcgaacggtgtagcgaaactggacatcgtggattcgcaaaacgcccaaccgacccagaacgagaaaatcgacctgttcatcagcaaaaagtacagcagcatgatcatcaaccccgttgatcgtaccgcagctggcgtcatcatcgacaaagccaaaaccgccaataccccggtggttttcctgaatcgcgaaccgattgcggaagacatgaacaaatgggacaaggtgtactatgtcggtgccaaagcggaggaaagcggtaccatcagcggtcagctgatcgtggactactggaaagcgaacccgaaagccgacaagaacggagatggcaaactgcagtacgtgctgttgcagggtgaaccgggtcatcaggatgccgaactgcgtaccaagttcagcgttcaggctatccaggatgcaggcatcgaggtggaagcgttagcggtggataccgctatgtgggatcgtgtgaaagggcaggaaaagatgcagaccttccttgcgtctcatggcgacaaaatcgaagcggttctggcgaacaacgacgatatggcgttaggagcgattgaggccttgaaagctgcaggctacttcagtggcgataagtacatgccggtggttggtgtggatgcaaccgctccagccgttcaggcgctggaagatggcacattgctcggaaccgtgctcaacgacgcgaaaagccagggcaaagcgagtgttgcgatagcagcggcgctttcgaagggtgaagcgccgaacaaagagaacaccggtttcgacatcaccgatgggaagtacgtgtggattgcgtacaagaagatcaccaaagataatattgcagatgctaaaggcggcagccatcatcatcatcatcattaatgataaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 118 - DNA, 1340 bp, synthetic construct
  misc_feature 1..1340: note = ttGGBP11C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttgcaaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 119 - DNA, 1340 bp, synthetic construct
  misc_feature 1..1340: note = ttGGBP16C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgactgcttcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 120 - DNA, 1340 bp, synthetic construct
  misc_feature 1..1340: note = ttGGBP17C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 121 - DNA, 1340 bp, synthetic construct
  misc_feature 1..1340: note = ttGGBP42C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagtgctcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 122 - DNA, 1340 bp, synthetic construct
  misc_feature 1..1340: note = ttGGBP67C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaacccttgcgatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 123 - DNA, 1340 bp, synthetic construct
  misc_feature 1..1340: note = ttGGBP91C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaactgcgaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 124 - DNA, 1340 bp, synthetic construct
  misc_feature 1..1340: note = ttGGBP92C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccggtgccctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 125 - DNA, 1340 bp, synthetic construct
  misc_feature 1..1340: note = ttGGBP111C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaagtgcgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 126 - DNA, 1340 bp, synthetic construct
  misc_feature 1..1340: note = ttGGBP148C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggctgcccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 127 - DNA, 1340 bp, synthetic construct
  misc_feature 1..1340: note = ttGGBP151C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggtgccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 128 - DNA, 1340 bp, synthetic construct
  misc_feature 1..1340: note = ttGGBP152C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcactgcgacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 129 - DNA, 1340 bp, synthetic construct
  misc_feature 1..1340: note = ttGGBP181C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgcctgctgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 130 - DNA, 1340 bp, synthetic construct
  misc_feature 1..1340: note = ttGGBP182C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 131 - DNA, 1340 bp, synthetic construct
  misc_feature 1..1340: note = ttGGBP183C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattggtgccgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 132 - DNA, 1340 bp, synthetic construct
  misc_feature 1..1340: note = ttGGBP257C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtctgcaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 133 - DNA, 1340 bp, synthetic construct
  misc_feature 1..1340: note = ttGGBP259C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaactgcgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 134 - DNA, 1340 bp, synthetic construct
  misc_feature 1..1340: note = ttGGBP300C Exemplary Cysteine Scan Mutant Expression Construct (Table 3)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatattgcaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 135 - DNA, 1340 bp, synthetic construct
  misc_feature 1..1340: note = ttGGBP17C.1 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 136 - DNA, 1340 bp, synthetic construct
  misc_feature 1..1340: note = ttGGBP17C.2 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatccgaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcacccggacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 137 - DNA, 1340 bp, synthetic construct
  misc_feature 1..1340: note = ttGGBP17C.3 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacaactgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgcgatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 138 - DNA, 1340 bp, synthetic construct
  misc_feature 1..1340: note = ttGGBP17C.4 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcagcaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 139 - DNA, 1340 bp, synthetic construct
  misc_feature 1..1340: note = ttGGBP17C.5 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgcgacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 140 - DNA, 1340 bp, synthetic construct
  misc_feature 1..1340: note = ttGGBP17C.6 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgaaacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 141 - DNA, 1340 bp, synthetic construct
  misc_feature 1..1340: note = ttGGBP17C.7 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacaacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 142 - DNA, 1340 bp, synthetic construct
  misc_feature 1..1340: note = ttGGBP17C.8 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacaactgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 143 - DNA, 1340 bp, synthetic construct
  misc_feature 1..1340: note = ttGGBP17C.9 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacagctgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 144 - DNA, 1340 bp, synthetic construct
  misc_feature 1..1340: note = ttGGBP17C.10 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactgcggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 145 - DNA, 1340 bp, synthetic construct
  misc_feature 1..1340: note = ttGGBP17C.11 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccgcggccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 146 - DNA, 1340 bp, synthetic construct
  misc_feature 1..1340: note = ttGGBP17C.19 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctggatgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 147 - DNA, 1340 bp, synthetic construct
  misc_feature 1..1340: note = ttGGBP17C.20 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgagcgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 148 - DNA, 1340 bp, synthetic construct
  misc_feature 1..1340: note = ttGGBP17C.21 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctggcggacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 149 - DNA, 1340 bp, synthetic construct
  misc_feature 1..1340: note = ttGGBP17C.22 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacaacaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 150 - DNA, 1340 bp, synthetic construct
  misc_feature 1..1340: note = ttGGBP17C.23 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgaccagaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 151 - DNA, 1340 bp, synthetic construct
  misc_feature 1..1340: note = ttGGBP17C.24 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgaccgcaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 152 - DNA, 1340 bp, synthetic construct
  misc_feature 1..1340: note = ttGGBP17C.25 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacaaaaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 153 - DNA, 1340 bp, synthetic construct
  misc_feature 1..1340: note = ttGGBP17C.26 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgactggaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 154 - DNA, 1340 bp, synthetic construct
  misc_feature 1..1340: note = ttGGBP17C.27 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgactttaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 155 - DNA, 1340 bp, synthetic construct
  misc_feature 1..1340: note = ttGGBP17C.28 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgactataaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 156 - DNA, 1340 bp, synthetic construct
  misc_feature 1..1340: note = ttGGBP17C.29 Exemplary Affinity-Tuning mutant (17C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacatgcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgggatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacagcaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 157 - DNA, 1340 bp, synthetic construct
  misc_feature 1..1340: note = ttGGBP182C.2.0 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 158 - DNA, 1340 bp, synthetic construct
  misc_feature 1..1340: note = ttGGBP182C.2.1 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagacagcatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 159 - DNA, 1340 bp, synthetic construct
  misc_feature 1..1340: note = ttGGBP182C.2.3 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagacaacatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 160 - DNA, 1340 bp, synthetic construct
  misc_feature 1..1340: note = ttGGBP182C.2.4 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagacatgatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 161 - DNA, 1340 bp, synthetic construct
  misc_feature 1..1340: note = ttGGBP182C.2.5 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcagcaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 162 - DNA, 1340 bp, synthetic construct
  misc_feature 1..1340: note = ttGGBP182C.2.6 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggaaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 163 - DNA, 1340 bp, synthetic construct
  misc_feature 1..1340: note = ttGGBP182C.2.7 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggtttcaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 164 - DNA, 1340 bp, synthetic construct
  misc_feature 1..1340: note = ttGGBP182C.2.8 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacaacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 165 - DNA, 1340 bp, synthetic construct
  misc_feature 1..1340: note = ttGGBP182C.2.9 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagactttatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 166 - DNA, 1340 bp, synthetic construct
  misc_feature 1..1340: note = ttGGBP182C.3 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 167 - DNA, 1340 bp, synthetic construct
  misc_feature 1..1340: note = ttGGBP182C.4 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcgaaccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 168 - DNA, 1340 bp, synthetic construct
  misc_feature 1..1340: note = ttGGBP182C.5 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatccgaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcacccggacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 169 - DNA, 1340 bp, synthetic construct
  misc_feature 1..1340: note = ttGGBP182C.6 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacaacttcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgcgatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 170 - DNA, 1340 bp, synthetic construct
  misc_feature 1..1340: note = ttGGBP182C.7 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcagcccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 171 - DNA, 1340 bp, synthetic construct
  misc_feature 1..1340: note = ttGGBP182C.8 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacgacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaacaaagaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggcaaaccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 172 - DNA, 1340 bp, synthetic construct
  misc_feature 1..1340: note = ttGGBP182C.9 Exemplary Affinity-Tuning mutant (182C Background) Expression Construct (Table 6)
  source 1..1340: mol_type = other DNA
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatataccatgaaacaactcaatatcggtgtagctatttataaattcgacaacacattcatgactggggtccggaatgctatgaccgcagaagcccaaggcaaggccaagttaaacatggtagacagtcagaactcacaacctacacaaaatgatcaggtcgacctcttcatcacgaaaaaaatgaatgcgttggcaatcaaccctgtagatcgcaccgcagctgggactatcatcgacaaggcaaagcaagcaaatatccctgtagtgttcttcaaccgggaacctttaccagaagacatgaaaaaatgggataaggtatattacgtaggcgcaaaggccgaacagagtggcattctccaaggtcaaatcatggctgattattggaaagctcatccggaagcggacaagaaccacgacggggttatgcaatatgtcatgttaatgggccagccggggcaccaagacgcaatcttacgtacgcaatactcgatccaaaccgtgaaagatgcaggcatcaaggtccaggagctggctaaagactacgccaattgcgatcgtgtcaccgctcatgacaaaatggctgcttggctctcgtcctttggcgacaagattgaagccgtttttgcaaataacgacgatatggccctgggtgccattgaagccctcaagtctgctggctatttcacgggcaacaagtacatcccagttgtaggcgtcgacgccaccgccccagggatccaggcgatcaaagacggtacattactggggacagtcctgaacgacgccaaaaaccaggcgaaagccactttcaacattgcatacgaacttgcacaagggatcacgccaaccaaagataacatcggttacgacatcaccgacggcaaatacgtttggattccatataaaaaaattacaaaagacaacatttcggatgcagagcaaggtggttcacatcatcatcatcatcattaatgaaagggcgatatccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 173 - protein, 321 aa, synthetic construct; identical to SEQ ID NO: 88
  REGION 1..321: note = ttGGBP182C.2.0_Imm0

SEQ ID NO: 174 - protein, 321 aa, synthetic construct; identical to SEQ ID NO: 88
  REGION 1..321: note = ttGBP,R91K,Q148E

SEQ ID NO: 175 - protein, 333 aa, synthetic construct
  REGION 1..333: note = ttGGBP182C.2.0_Imm1
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSGGSKKKKKKGGSHHHHHH

SEQ ID NO: 176 - protein, 333 aa, synthetic construct
  REGION 1..333: note = ttGGBP182C.2.0_Imm2
MHHHHHHGGSKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSGGSKKKKKK

SEQ ID NO: 177 - protein, 333 aa, synthetic construct
  REGION 1..333: note = ttGGBP182C.2.0_Imm3
MKKKKKKGGSGGSKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 178 - protein, 359 aa, synthetic construct
  REGION 1..359: note = ttGGBP182C.2.0_Imm4
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSGGSTGEKPYKCPECGKSFSRSDHLSRHQRTHQNKKGGSHHHHHH

SEQ ID NO: 179 - protein, 359 aa, synthetic construct
  REGION 1..359: note = ttGGBP182C.2.0_Imm5
MTGEKPYKCPECGKSFSRSDHLSRHQRTHQNKKGGSGGSKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 180 - protein, 345 aa, synthetic construct
  REGION 1..345: note = ttGGBP182C.2.0_Imm6
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSGGSTGEKPYKCPECGKSFSRSGGSHHHHHH

SEQ ID NO: 181 - protein, 345 aa, synthetic construct
  REGION 1..345: note = ttGGBP182C.2.0_Imm7
MTGEKPYKCPECGKSFSRSGGSGGSKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQGGSHHHHHH

SEQ ID NO: 182 - protein, 9 aa, synthetic construct
  REGION 1..9: note = Conserved GGBP Sequence
  SITE 4: note = misc_feature - Xaa can be any naturally occurring amino acid
  SITE 7: note = misc_feature - Xaa can be any naturally occurring amino acid
IYKXDDXFM

SEQ ID NO: 183 - protein, 8 aa, synthetic construct
  REGION 1..8: note = Conserved GGBP Sequence
  SITE 3: note = misc_feature - Xaa can be any naturally occurring amino acid
  SITE 6: note = misc_feature - Xaa can be any naturally occurring amino acid
YKXDDXFM

SEQ ID NO: 184 - protein, 7 aa, synthetic construct
  REGION 1..7: note = Conserved GGBP Sequence
  SITE 3: note = misc_feature - Xaa can be any naturally occurring amino acid
  SITE 6: note = misc_feature - Xaa can be any naturally occurring amino acid
YKXDDXF

SEQ ID NO: 185 - protein, 6 aa, synthetic construct
  REGION 1..6: note = Conserved GGBP Sequence
  SITE 2: note = misc_feature - Xaa can be any naturally occurring amino acid
  SITE 5: note = misc_feature - Xaa can be any naturally occurring amino acid
KXDDXF

SEQ ID NO: 186 - protein, 5 aa, synthetic construct
  REGION 1..5: note = Conserved GGBP Sequence
  SITE 3: note = misc_feature - Xaa can be any naturally occurring amino acid
YKXDD

SEQ ID NO: 187-188 - skipped (2 entries)

SEQ ID NO: 189 - protein, 8 aa, synthetic construct
  REGION 1..8: note = Conserved GGBP Sequence
  SITE 2: note = misc_feature - Xaa can be any naturally occurring amino acid
  REGION 5..6: note = misc_feature - Xaa can be any naturally occurring amino acid
DXFMXXVR

SEQ ID NO: 190 - protein, 7 aa, synthetic construct
  REGION 1..7: note = Conserved GGBP Sequence
  SITE 2: note = misc_feature - Xaa can be any naturally occurring amino acid
  REGION 5..6: note = misc_feature - Xaa can be any naturally occurring amino acid
DXFMXXV

SEQ ID NO: 191 - skipped

SEQ ID NO: 192 - protein, 9 aa, synthetic construct
  REGION 1..9: note = Conserved GGBP Sequence
  SITE 4: note = misc_feature - Xaa can be any naturally occurring amino acid
  SITE 7: note = misc_feature - Xaa can be any naturally occurring amino acid
IYKXDNXFM

SEQ ID NO: 193 - protein, 8 aa, synthetic construct
  REGION 1..8: note = Conserved GGBP Sequence
  SITE 3: note = misc_feature - Xaa can be any naturally occurring amino acid
  SITE 6: note = misc_feature - Xaa can be any naturally occurring amino acid
YKXDNXFM

SEQ ID NO: 194 - protein, 7 aa, synthetic construct
  REGION 1..7: note = Conserved GGBP Sequence
  SITE 3: note = misc_feature - Xaa can be any naturally occurring amino acid
  SITE 6: note = misc_feature - Xaa can be any naturally occurring amino acid
YKXDNXF

SEQ ID NO: 195 - protein, 6 aa, synthetic construct
  REGION 1..6: note = Conserved GGBP Sequence
  SITE 2: note = misc_feature - Xaa can be any naturally occurring amino acid
  SITE 5: note = misc_feature - Xaa can be any naturally occurring amino acid
KXDNXF

SEQ ID NO: 196 - protein, 5 aa, synthetic construct
  REGION 1..5: note = Conserved GGBP Sequence
  SITE 3: note = misc_feature - Xaa can be any naturally occurring amino acid
YKXDN

SEQ ID NO: 197-198 - skipped (2 entries)

SEQ ID NO: 199 - protein, 8 aa, synthetic construct
  REGION 1..8: note = Conserved GGBP Sequence
  SITE 2: note = misc_feature - Xaa can be any naturally occurring amino acid
  REGION 5..6: note = misc_feature - Xaa can be any naturally occurring amino acid
NXFMXXVR

SEQ ID NO: 200 - protein, 7 aa, synthetic construct
  REGION 1..7: note = Conserved GGBP Sequence
  SITE 2: note = misc_feature - Xaa can be any naturally occurring amino acid
  REGION 5..6: note = misc_feature - Xaa can be any naturally occurring amino acid
NXFMXXV

SEQ ID NO: 201 - skipped

SEQ ID NO: 202 - protein, 9 aa, synthetic construct
  REGION 1..9: note = Conserved GGBP Sequence
PVVFFNKEP

SEQ ID NO: 203 - protein, 9 aa, synthetic construct
  REGION 1..9: note = Conserved GGBP Sequence
  SITE 5: note = misc_feature - Xaa can be any naturally occurring amino acid
PVVFXNKEP

SEQ ID NO: 204 - protein, 9 aa, synthetic construct
  REGION 1..9: note = Conserved GGBP Sequence
  SITE 7: note = misc_feature - Xaa can be any naturally occurring amino acid
PVVFFNXEP

SEQ ID NO: 205 - protein, 9 aa, synthetic construct
  REGION 1..9: note = Conserved GGBP Sequence
  SITE 5: note = misc_feature - Xaa can be any naturally occurring amino acid
  SITE 7: note = misc_feature - Xaa can be any naturally occurring amino acid
PVVFXNXEP

SEQ ID NO: 206 - protein, 8 aa, synthetic construct
  REGION 1..8: note = Conserved GGBP Sequence
  SITE 4: note = misc_feature - Xaa can be any naturally occurring amino acid
  SITE 6: note = misc_feature - Xaa can be any naturally occurring amino acid
VVFXNXEP

SEQ ID NO: 207 - protein, 7 aa, synthetic construct
  REGION 1..7: note = Conserved GGBP Sequence
  SITE 3: note = misc_feature - Xaa can be any naturally occurring amino acid
  SITE 5: note = misc_feature - Xaa can be any naturally occurring amino acid
VFXNXEP

SEQ ID NO: 208 - protein, 8 aa, synthetic construct
  REGION 1..8: note = Conserved GGBP Sequence
  SITE 5: note = misc_feature - Xaa can be any naturally occurring amino acid
  SITE 7: note = misc_feature - Xaa can be any naturally occurring amino acid
PVVFXNXE

SEQ ID NO: 209 - protein, 6 aa, synthetic construct
  REGION 1..6: note = Conserved GGBP Sequence
  SITE 5: note = misc_feature - Xaa can be any naturally occurring amino acid
PVVFXN

SEQ ID NO: 210 - protein, 6 aa, synthetic construct
  REGION 1..6: note = Conserved GGBP Sequence
  SITE 2: note = misc_feature - Xaa can be any naturally occurring amino acid
  SITE 5: note = misc_feature - Xaa can be any naturally occurring amino acid
PXVFXN

SEQ ID NO: 211 - protein, 6 aa, synthetic construct
  REGION 1..6: note = Conserved GGBP Sequence
  SITE 3: note = misc_feature - Xaa can be any naturally occurring amino acid
  SITE 5: note = misc_feature - Xaa can be any naturally occurring amino acid
PVXFXN

SEQ ID NO: 212 - protein, 6 aa, synthetic construct
  REGION 1..6: note = Conserved GGBP Sequence
  SITE 2: note = misc_feature - Xaa can be any naturally occurring amino acid
  SITE 4: note = misc_feature - Xaa can be any naturally occurring amino acid
FXNXEP

SEQ ID NO: 213 - skipped

SEQ ID NO: 214 - protein, 10 aa, synthetic construct
  REGION 1..10: note = Conserved GGBP Sequence
PGHPDAEART

SEQ ID NO: 215 - protein, 10 aa, synthetic construct
  REGION 1..10: note = Conserved GGBP Sequence
  SITE 4: note = misc_feature - Xaa can be any naturally occurring amino acid
  REGION 7..8: note = misc_feature - Xaa can be any naturally occurring amino acid
PGHXDAXXRT

SEQ ID NO: 216 - protein, 9 aa, synthetic construct
  REGION 1..9: note = Conserved GGBP Sequence
  SITE 3: note = misc_feature - Xaa can be any naturally occurring amino acid
  REGION 6..7: note = misc_feature - Xaa can be any naturally occurring amino acid
GHXDAXXRT

SEQ ID NO: 217 - protein, 8 aa, synthetic construct
  REGION 1..8: note = Conserved GGBP Sequence
  SITE 2: note = misc_feature - Xaa can be any naturally occurring amino acid
  REGION 5..6: note = misc_feature - Xaa can be any naturally occurring amino acid
HXDAXXRT

SEQ ID NO: 218 - protein, 6 aa, synthetic construct
  REGION 1..6: note = Conserved GGBP Sequence
  REGION 3..4: note = misc_feature - Xaa can be any naturally occurring amino acid
DAXXRT

SEQ ID NO: 219 - protein, 9 aa, synthetic construct
  REGION 1..9: note = Conserved GGBP Sequence
  SITE 4: note = misc_feature - Xaa can be any naturally occurring amino acid
  REGION 7..8: note = misc_feature - Xaa can be any naturally occurring amino acid
PGHXDAXXR

SEQ ID NO: 220 - protein, 6 aa, synthetic construct
  REGION 1..6: note = Conserved GGBP Sequence
  SITE 4: note = misc_feature - Xaa can be any naturally occurring amino acid
PGHXDA

SEQ ID NO: 221 - protein, 5 aa, synthetic construct
  REGION 1..5: note = Conserved GGBP Sequence
  SITE 4: note = misc_feature - Xaa can be any naturally occurring amino acid
PGHXD

SEQ ID NO: 222 - protein, 10 aa, synthetic construct
  REGION 1..10: note = Conserved GGBP Sequence
PGNPDAEART

SEQ ID NO: 223 - protein, 10 aa, synthetic construct
  REGION 1..10: note = Conserved GGBP Sequence
  SITE 4: note = misc_feature - Xaa can be any naturally occurring amino acid
  REGION 7..8: note = misc_feature - Xaa can be any naturally occurring amino acid
PGNXDAXXRT

SEQ ID NO: 224 - protein, 9 aa, synthetic construct
  REGION 1..9: note = Conserved GGBP Sequence
  SITE 3: note = misc_feature - Xaa can be any naturally occurring amino acid
  REGION 6..7: note = misc_feature - Xaa can be any naturally occurring amino acid
GNXDAXXRT

SEQ ID NO: 225 - protein, 8 aa, synthetic construct
  REGION 1..8: note = Conserved GGBP Sequence
  SITE 2: note = misc_feature - Xaa can be any naturally occurring amino acid
  REGION 5..6: note = misc_feature - Xaa can be any naturally occurring amino acid
NXDAXXRT

SEQ ID NO: 226 - protein, 9 aa, synthetic construct
  REGION 1..9: note = Conserved GGBP Sequence
  SITE 4: note = misc_feature - Xaa can be any naturally occurring amino acid
  REGION 7..8: note = misc_feature - Xaa can be any naturally occurring amino acid
PGNXDAXXR

SEQ ID NO: 227 - protein, 6 aa, synthetic construct
  REGION 1..6: note = Conserved GGBP Sequence
  SITE 4: note = misc_feature - Xaa can be any naturally occurring amino acid
PGNXDA

SEQ ID NO: 228 - protein, 5 aa, synthetic construct
  REGION 1..5: note = Conserved GGBP Sequence
  SITE 4: note = misc_feature - Xaa can be any naturally occurring amino acid
PGNXD

SEQ ID NO: 229 - protein, 6 aa, synthetic construct
  REGION 1..6: note = Conserved GGBP Sequence
DTAMWD

SEQ ID NO: 230 - protein, 6 aa, synthetic construct
  REGION 1..6: note = DTAMCD (Conserved Sequence)
DTAMCD

SEQ ID NO: 231 - protein, 5 aa, synthetic construct
  REGION 1..5: note = Conserved GGBP Sequence
DTAMW

SEQ ID NO: 232 - protein, 5 aa, synthetic construct
  REGION 1..5: note = Conserved GGBP Sequence
DTAMC

SEQ ID NO: 233 - protein, 5 aa, synthetic construct
  REGION 1..5: note = Conserved GGBP Sequence
TAMWD

SEQ ID NO: 234 - protein, 5 aa, synthetic construct
  REGION 1..5: note = Conserved GGBP Sequence
TAMCD

SEQ ID NO: 235-236 - skipped (2 entries)

SEQ ID NO: 237 - protein, 9 aa, synthetic construct
  REGION 1..9: note = Conserved GGBP Sequence
IEVVIANND

SEQ ID NO: 238 - protein, 8 aa, synthetic construct
  REGION 1..8: note = Conserved GGBP Sequence
EVVIANND

SEQ ID NO: 239 - protein, 8 aa, synthetic construct
  REGION 1..8: note = Conserved GGBP Sequence
IEVVIANN

SEQ ID NO: 240 - protein, 7 aa, synthetic construct
  REGION 1..7: note = Conserved GGBP Sequence
EVVIANN

SEQ ID NO: 241 - protein, 9 aa, synthetic construct
  REGION 1..9: note = Conserved GGBP Sequence
  SITE 3: note = misc_feature - Xaa can be any naturally occurring amino acid
  REGION 5..6: note = misc_feature - Xaa can be any naturally occurring amino acid
IEXVXXNND

SEQ ID NO: 242 - protein, 8 aa, synthetic construct
  REGION 1..8: note = Conserved GGBP Sequence
  SITE 3: note = misc_feature - Xaa can be any naturally occurring amino acid
  REGION 5..6: note = misc_feature - Xaa can be any naturally occurring amino acid
IEXVXXNN

SEQ ID NO: 243 - protein, 8 aa, synthetic construct
  REGION 1..8: note = Conserved GGBP Sequence
  SITE 2: note = misc_feature - Xaa can be any naturally occurring amino acid
  REGION 4..5: note = misc_feature - Xaa can be any naturally occurring amino acid
EXVXXNND

SEQ ID NO: 244 - protein, 7 aa, synthetic construct
  REGION 1..7: note = Conserved GGBP Sequence
  SITE 2: note = misc_feature - Xaa can be any naturally occurring amino acid
  REGION 4..5: note = misc_feature - Xaa can be any naturally occurring amino acid
EXVXXNN

SEQ ID NO: 245 - protein, 7 aa, synthetic construct
  REGION 1..7: note = Conserved GGBP Sequence
PVFGVDA

SEQ ID NO: 246 - protein, 6 aa, synthetic construct
  REGION 1..6: note = Conserved GGBP Sequence
VFGVDA

SEQ ID NO: 247 - protein, 6 aa, synthetic construct
  REGION 1..6: note = Conserved GGBP Sequence
PVFGVD

SEQ ID NO: 248 - protein, 5 aa, synthetic construct
  REGION 1..5: note = Conserved GGBP Sequence
FGVDA

SEQ ID NO: 249 - protein, 7 aa, synthetic construct
  REGION 1..7: note = Conserved GGBP Sequence
  SITE 3: note = misc_feature - Xaa can be any naturally occurring amino acid
PVXGVDA

SEQ ID NO: 250 - protein, 6 aa, synthetic construct
  REGION 1..6: note = Conserved GGBP Sequence
  SITE 2: note = misc_feature - Xaa can be any naturally occurring amino acid
VXGVDA

SEQ ID NO: 251 - protein, 6 aa, synthetic construct
  REGION 1..6: note = Conserved GGBP Sequence
  SITE 3: note = misc_feature - Xaa can be any naturally occurring amino acid
PVXGVD

SEQ ID NO: 252 - protein, 5 aa, synthetic construct
  REGION 1..5: note = Conserved GGBP Sequence
  SITE 2: note = misc_feature - Xaa can be any naturally occurring amino acid
VXGVD

SEQ ID NO: 253 - protein, 7 aa, synthetic construct
  REGION 1..7: note = Conserved GGBP Sequence
GTVLNDA

SEQ ID NO: 254 - protein, 6 aa, synthetic construct
  REGION 1..6: note = Conserved GGBP Sequence
GTVLND

SEQ ID NO: 255 - protein, 5 aa, synthetic construct
  REGION 1..5: note = Conserved GGBP Sequence
GTVLN

SEQ ID NO: 256 - protein, 5 aa, synthetic construct
  REGION 1..5: note = Conserved GGBP Sequence
TVLND

SEQ ID NO: 257 - protein, 325 aa, synthetic construct
  REGION 1..325: note = chyGGBP_F12C (chGGBP with signal sequence replaced with M; F12C, C39A, and C206A mutations; and GGSHHHHHH at C-terminus)
MKPYIGVAIYKCDDTFMTGVRNAIAKEGEGKAKLDFVDAQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHNGIMEYVMITGEPGHQDAILRTEYSIKAVEAAGIKTKALAQDTAMWDRVKGQEKMQAFLASFGDRIEAVFANNDDMALGAIEALKAAGYFKNGKYIPVVGVDATTPGLQALEEGTLLGTVLNDAKAQGKATFNLAYVLAKGEKPTKENVGFDITDGKYIWVPYQKVTKDNLEEMKKYVNEQGGSHHHHHH

SEQ ID NO: 258 - protein, 325 aa, synthetic construct
  REGION 1..325: note = chyGGBP_F16C (chGGBP with signal sequence replaced with M; F16C, C39A, and C206A mutations; and GGSHHHHHH at C-terminus)
MKPYIGVAIYKFDDTCMTGVRNAIAKEGEGKAKLDFVDAQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHNGIMEYVMITGEPGHQDAILRTEYSIKAVEAAGIKTKALAQDTAMWDRVKGQEKMQAFLASFGDRIEAVFANNDDMALGAIEALKAAGYFKNGKYIPVVGVDATTPGLQALEEGTLLGTVLNDAKAQGKATFNLAYVLAKGEKPTKENVGFDITDGKYIWVPYQKVTKDNLEEMKKYVNEQGGSHHHHHH

SEQ ID NO: 259 - protein, 325 aa, synthetic construct; identical to SEQ ID NO: 48
  REGION 1..325: note = chyGGBP_W181C (chGGBP with signal sequence replaced with M; W181C, C39A, and C206A mutations; and GGSHHHHHH at C-terminus)

SEQ ID NO: 260 - protein, 325 aa, synthetic construct
  REGION 1..325: note = cobGGBP_F12C (cobGGBP with signal sequence replaced with M; F12C, C39A, C173A, and C206A mutations; and GGSHHHHHH at C-terminus)
MKPYIGVAIYKCDDTFMTGVRNAIAKEGEGKAKLDFVDAQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHDGIMQYVMITGEPGHQDAILRTEYSIKAVEAAGIRVKALAQDTAMWDRVKGQEKMQAFLASFGDKIEAVFANNDDMALGAIEALKAAGYFKDGKYVPVVGVDATTPGLQALEEGTLLGTVLNDAKAQGKATFNLAYVLAKGEKPTKENVGFEITDGKYIWVPYQKVTKDNLEEMKKYVNEQGGSHHHHHH

SEQ ID NO: 261 - protein, 325 aa, synthetic construct
  REGION 1..325: note = cobGGBP_F16C (cobGGBP with signal sequence replaced with M; F16C, C39A, C173A, and C206A mutations; and GGSHHHHHH at C-terminus)
MKPYIGVAIYKFDDTCMTGVRNAIAKEGEGKAKLDFVDAQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHDGIMQYVMITGEPGHQDAILRTEYSIKAVEAAGIRVKALAQDTAMWDRVKGQEKMQAFLASFGDKIEAVFANNDDMALGAIEALKAAGYFKDGKYVPVVGVDATTPGLQALEEGTLLGTVLNDAKAQGKATFNLAYVLAKGEKPTKENVGFEITDGKYIWVPYQKVTKDNLEEMKKYVNEQGGSHHHHHH

SEQ ID NO: 262 - protein, 325 aa, synthetic construct
  REGION 1..325: note = cobGGBP_W181C (cobGGBP with signal sequence replaced with M, W181C, C39A, C173A, and C206A mutations; and GGSHHHHHH at C-terminus)
MKPYIGVAIYKFDDTFMTGVRNAIAKEGEGKAKLDFVDAQNSQSTQNDKIDLFITKKVDALAINPVDRTAAGVLIDKAKQANIPVVFFNREPLPEDMKKWDKVYYVGAKAEQSGTLQGEIMAEYWKSHPEADKNHDGIMQYVMITGEPGHQDAILRTEYSIKAVEAAGIRVKALAQDTAMCDRVKGQEKMQAFLASFGDKIEAVFANNDDMALGAIEALKAAGYFKDGKYVPVVGVDATTPGLQALEEGTLLGTVLNDAKAQGKATFNLAYVLAKGEKPTKENVGFEITDGKYIWVPYQKVTKDNLEEMKKYVNEQGGSHHHHHH

SEQ ID NO: 263 - protein, 316 aa, synthetic construct
  REGION 1..316: note = pspGGPB_F13C (pspGGBP with signal sequence replaced with M; F13C mutation; and GGSHHHHHH at C-terminus)
MVGVAIYKFDDTCMTGVRNAMSDAANGVAKLDIVDSQNAQPTQNEKIDLFISKKYSSMIINPVDRTAAGVIIDKAKTANTPVVFLNREPIAEDMNKWDKVYYVGAKAEESGTISGQLIVDYWKANPKADKNGDGKLQYVLLQGEPGHQDAELRTKFSVQAIQDAGIEVEALAVDTAMWDRVKGQEKMQTFLASHGDKIEAVLANNDDMALGAIEALKAAGYFSGDKYMPVVGVDATAPAVQALEDGTLLGTVLNDAKSQGKASVAIAAALSKGEAPNKENTGFDITDGKYVWIAYKKITKDNIADAKGGSHHHHHH

SEQ ID NO: 264 - protein, 316 aa, synthetic construct
  REGION 1..316: note = pspGGPB_F9C (pspGGBP with signal sequence replaced with M; F9C mutation; and GGSHHHHHH at C-terminus)
MVGVAIYKCDDTFMTGVRNAMSDAANGVAKLDIVDSQNAQPTQNEKIDLFISKKYSSMIINPVDRTAAGVIIDKAKTANTPVVFLNREPIAEDMNKWDKVYYVGAKAEESGTISGQLIVDYWKANPKADKNGDGKLQYVLLQGEPGHQDAELRTKFSVQAIQDAGIEVEALAVDTAMWDRVKGQEKMQTFLASHGDKIEAVLANNDDMALGAIEALKAAGYFSGDKYMPVVGVDATAPAVQALEDGTLLGTVLNDAKSQGKASVAIAAALSKGEAPNKENTGFDITDGKYVWIAYKKITKDNIADAKGGSHHHHHH

SEQ ID NO: 265 - protein, 316 aa, synthetic construct
  REGION 1..316: note = pspGGPB_W178C (pspGGBP with signal sequence replaced with M; W178C mutation; and GGSHHHHHH at C-terminus)
MVGVAIYKFDDTFMTGVRNAMSDAANGVAKLDIVDSQNAQPTQNEKIDLFISKKYSSMIINPVDRTAAGVIIDKAKTANTPVVFLNREPIAEDMNKWDKVYYVGAKAEESGTISGQLIVDYWKANPKADKNGDGKLQYVLLQGEPGHQDAELRTKFSVQAIQDAGIEVEALAVDTAMCDRVKGQEKMQTFLASHGDKIEAVLANNDDMALGAIEALKAAGYFSGDKYMPVVGVDATAPAVQALEDGTLLGTVLNDAKSQGKASVAIAAALSKGEAPNKENTGFDITDGKYVWIAYKKITKDNIADAKGGSHHHHHH

SEQ ID NO: 266 - protein, 316 aa, synthetic construct
  REGION 1..316: note = csaGGBP_F18C (csaGGBP with signal sequence replaced with M; F18C, C62A,C82A,C113A, and C211A mutations; and GGSHHHHHH at C-terminus)
METGPKIGVSIYRYDDTCMKLYRQELKQYLEETYHAEVIMRNAGGDQKEQDKQVNQFISDGADGIIVNPVEIPAAQELADAASRAGIPLVFINREPKEEEQKRWREKQMAVSAVGTDSRQAGTYQGEIILETLNKGDFNGDGVVSYVMLMGEKGNEDSQYRTEYSIKALEEGGMKTEELFSGNGNWNKDEGKKLAKQALASWGNRIEVFFANNDSMANGALEAVEEAGRIPGKDIYLVGVDALQDTVTYIKEGRMTGTVLNDHEGQSQMAADTLKKMIDGESVETRYQVDYIKVTAISTFQTLKGEDGGSHHHHHH

SEQ ID NO: 267 - protein, 316 aa, synthetic construct
  REGION 1..316: note = csaGGBP_W186C (csaGGBP with signal sequence replaced with M; W186C, C62A,C82A,C113A, and C211A mutations; and GGSHHHHHH at C-terminus)
METGPKIGVSIYRYDDTFMKLYRQELKQYLEETYHAEVIMRNAGGDQKEQDKQVNQFISDGADGIIVNPVEIPAAQELADAASRAGIPLVFINREPKEEEQKRWREKQMAVSAVGTDSRQAGTYQGEIILETLNKGDFNGDGVVSYVMLMGEKGNEDSQYRTEYSIKALEEGGMKTEELFSGNGNCNKDEGKKLAKQALASWGNRIEVFFANNDSMANGALEAVEEAGRIPGKDIYLVGVDALQDTVTYIKEGRMTGTVLNDHEGQSQMAADTLKKMIDGESVETRYQVDYIKVTAISTFQTLKGEDGGSHHHHHH

SEQ ID NO: 268 - protein, 316 aa, synthetic construct
  REGION 1..316: note = csaGGBP_Y14C (csaGGBP with signal sequence replaced with M; Y14C, C62A,C82A,C113A, and C211A mutations; and GGSHHHHHH at C-terminus)
METGPKIGVSIYRCDDTFMKLYRQELKQYLEETYHAEVIMRNAGGDQKEQDKQVNQFISDGADGIIVNPVEIPAAQELADAASRAGIPLVFINREPKEEEQKRWREKQMAVSAVGTDSRQAGTYQGEIILETLNKGDFNGDGVVSYVMLMGEKGNEDSQYRTEYSIKALEEGGMKTEELFSGNGNWNKDEGKKLAKQALASWGNRIEVFFANNDSMANGALEAVEEAGRIPGKDIYLVGVDALQDTVTYIKEGRMTGTVLNDHEGQSQMAADTLKKMIDGESVETRYQVDYIKVTAISTFQTLKGEDGGSHHHHHH

SEQ ID NO: 269 - protein, 323 aa, synthetic construct
  REGION 1..323: note = bprGGBP_F16C (bprGGBP with signal sequence replaced with M; F16C, C8A, C112A, C116A, C179A, C211A, and C289A mutations; and GGSHHHHHH at C-terminus)
MDAKVGVAIYQKSDNCMSLFSSELVKYLVSRGFSKDNIILYDSNNDENVQLSQVEELIASGINALIINPVNSSVAHSITDMASASNIPLVYINREPSGDEENRWEMYQLNVAYVGADARQSGIYQGEILLSLGKNKLDHNGDGKIQYFMIEGAPENIDAGYRTLYSVSALQNSEMEMDALLDEVGNWDETTASLLVSKGIQNGLKPEVIIANNDAMALGAIKAAEKSGLVPGEDVYIVGVDALPEAIEMIKAGKLAGTVYNDYVLQSHKSADAVINYLKGIDNEHYIGADYVKVDIDNAESIAGLTNTDEEDIDGGSHHHHHH

SEQ ID NO: 270 - protein, 323 aa, synthetic construct
  REGION 1..323: note = bprGGBP_K12C (bprGGBP with signal sequence replaced with M; K12C, C8A, C112A, C116A, C179A, C211A, and C289A mutations; and GGSHHHHHH at C-terminus)
MDAKVGVAIYQCSDNFMSLFSSELVKYLVSRGFSKDNIILYDSNNDENVQLSQVEELIASGINALIINPVNSSVAHSITDMASASNIPLVYINREPSGDEENRWEMYQLNVAYVGADARQSGIYQGEILLSLGKNKLDHNGDGKIQYFMIEGAPENIDAGYRTLYSVSALQNSEMEMDALLDEVGNWDETTASLLVSKGIQNGLKPEVIIANNDAMALGAIKAAEKSGLVPGEDVYIVGVDALPEAIEMIKAGKLAGTVYNDYVLQSHKSADAVINYLKGIDNEHYIGADYVKVDIDNAESIAGLTNTDEEDIDGGSHHHHHH

SEQ ID NO: 271 - protein, 323 aa, synthetic construct
  REGION 1..323: note = bprGGBP_W187C (bprGGBP with signal sequence replaced with M; W187C, C8A, C112A, C116A, C179A, C211A, and C289A mutations; and GGSHHHHHH at C-terminus)
MDAKVGVAIYQKSDNFMSLFSSELVKYLVSRGFSKDNIILYDSNNDENVQLSQVEELIASGINALIINPVNSSVAHSITDMASASNIPLVYINREPSGDEENRWEMYQLNVAYVGADARQSGIYQGEILLSLGKNKLDHNGDGKIQYFMIEGAPENIDAGYRTLYSVSALQNSEMEMDALLDEVGNCDETTASLLVSKGIQNGLKPEVIIANNDAMALGAIKAAEKSGLVPGEDVYIVGVDALPEAIEMIKAGKLAGTVYNDYVLQSHKSADAVINYLKGIDNEHYIGADYVKVDIDNAESIAGLTNTDEEDIDGGSHHHHHH

SEQ ID NO: 272 - protein, 315 aa, synthetic construct
  REGION 1..315: note = rinGGBP_A_F10C (rinGGBP_A with signal sequence replaced with M; F10C, C6A, C114A, C177A, C210A, and C288A mutations; and GGSHHHHHH at C-terminus)
MKVGVAIYQCSDNFMTLFRTELENYLVEKGFSKDNITIVDGANDQATQTGQIDNFITEGVDVLIINPVNSSSAATITDKVVAAGIPLVYINREPDEEEQKRWSDNNWDVTYVGADARQSGTFQGEMISDLGLDTVDLNGNGKIDYVMVEGDPENVDAQYRTEYSVKALEDAGLEVNALSDQVGNWQQDQAQQIVANALGQYGNDVEVVFANNDAMALGALQAIQSAGRTVGTDIYLVGVDALSEALEDVLAGTMTGTVFNDHFSQSHSAADAAINYITGAGNDHYIGADYVKVTKDNAQDVLDMVKGGSHHHHHH

SEQ ID NO: 273 - protein, 315 aa, synthetic construct
  REGION 1..315: note = rinGGBP_A_F14C (rinGGBP_A with signal sequence replaced with M; F14C, C6A, C114A, C177A, C210A, and C288A mutations; and GGSHHHHHH at C-terminus)
MKVGVAIYQFSDNCMTLFRTELENYLVEKGFSKDNITIVDGANDQATQTGQIDNFITEGVDVLIINPVNSSSAATITDKVVAAGIPLVYINREPDEEEQKRWSDNNWDVTYVGADARQSGTFQGEMISDLGLDTVDLNGNGKIDYVMVEGDPENVDAQYRTEYSVKALEDAGLEVNALSDQVGNWQQDQAQQIVANALGQYGNDVEVVFANNDAMALGALQAIQSAGRTVGTDIYLVGVDALSEALEDVLAGTMTGTVFNDHFSQSHSAADAAINYITGAGNDHYIGADYVKVTKDNAQDVLDMVKGGSHHHHHH

SEQ ID NO: 274 - protein, 315 aa, synthetic construct
  REGION 1..315: note = rinGGBP_A_W185C (rinGGBP_A with signal sequence replaced with M; W185C, C6A, C114A, C177A, C210A, and C288A mutations; and GGSHHHHHH at C-terminus)
MKVGVAIYQFSDNFMTLFRTELENYLVEKGFSKDNITIVDGANDQATQTGQIDNFITEGVDVLIINPVNSSSAATITDKVVAAGIPLVYINREPDEEEQKRWSDNNWDVTYVGADARQSGTFQGEMISDLGLDTVDLNGNGKIDYVMVEGDPENVDAQYRTEYSVKALEDAGLEVNALSDQVGNCQQDQAQQIVANALGQYGNDVEVVFANNDAMALGALQAIQSAGRTVGTDIYLVGVDALSEALEDVLAGTMTGTVFNDHFSQSHSAADAAINYITGAGNDHYIGADYVKVTKDNAQDVLDMVKGGSHHHHHH

SEQ ID NO: 275 - protein, 327 aa, synthetic construct
  REGION 1..327: note = rinGGBP_B_F17C (rinGGBP_B with signal sequence replaced with M; F17C, C66A, C70A, and C306A; and GGSHHHHHH at C-terminus)
MKSIKIGISVYDQYDTCVSEMMKDFNDYATKKEEETGVAINIDTYNASASQSTQNSQVENMITEGADVIAVNLVDRTDPTAIIDLAEKNNIPVIFFNRELVEEDLERWTRLYYVGAQAFESGIMQGELAAEAFLTDQSLDKNGDGIFQYVVLEGEAGHQDAIVRTEYSVSTMIDSGVEVEKLGYAIANWNRAQAQTKMAQLMSQFGDSIELVIANNDDMALGAIDALKASGLTKDEWPAVIGIDGTDVGLEAVKNKEMIGTVYNDKEGQADAMLNLAYELSTGSDLSDLNLIDGKYIRLPYARVTADDVDSYMEGDTEGGSHHHHHH

SEQ ID NO: 276 - protein, 327 aa, synthetic construct
  REGION 1..327: note = rinGGBP_B_Q13C (rinGGBP_B with signal sequence replaced with M; Q13C, C66A, C70A, and C306A mutations; and GGSHHHHHH at C-terminus)
MKSIKIGISVYDCYDTFVSEMMKDFNDYATKKEEETGVAINIDTYNASASQSTQNSQVENMITEGADVIAVNLVDRTDPTAIIDLAEKNNIPVIFFNRELVEEDLERWTRLYYVGAQAFESGIMQGELAAEAFLTDQSLDKNGDGIFQYVVLEGEAGHQDAIVRTEYSVSTMIDSGVEVEKLGYAIANWNRAQAQTKMAQLMSQFGDSIELVIANNDDMALGAIDALKASGLTKDEWPAVIGIDGTDVGLEAVKNKEMIGTVYNDKEGQADAMLNLAYELSTGSDLSDLNLIDGKYIRLPYARVTADDVDSYMEGDTEGGSHHHHHH

SEQ ID NO: 277 - protein, 327 aa, synthetic construct
  REGION 1..327: note = rinGGBP_B_W189C (rinGGBP_B with signal sequence replaced with M; W189C, C66A, C70A, and C306A mutations; and GGSHHHHHH at C-terminus)
MKSIKIGISVYDQYDTFVSEMMKDFNDYATKKEEETGVAINIDTYNASASQSTQNSQVENMITEGADVIAVNLVDRTDPTAIIDLAEKNNIPVIFFNRELVEEDLERWTRLYYVGAQAFESGIMQGELAAEAFLTDQSLDKNGDGIFQYVVLEGEAGHQDAIVRTEYSVSTMIDSGVEVEKLGYAIANCNRAQAQTKMAQLMSQFGDSIELVIANNDDMALGAIDALKASGLTKDEWPAVIGIDGTDVGLEAVKNKEMIGTVYNDKEGQADAMLNLAYELSTGSDLSDLNLIDGKYIRLPYARVTADDVDSYMEGDTEGGSHHHHHH

SEQ ID NO: 278 - protein, 309 aa, synthetic construct
  REGION 1..309: note = fprGGBP_F12C (fprGGBP with signal sequence replaced with M; F12C, C8A, C105A, C106A, C143A, and C205A mutations; and GGSHHHHHH at C-terminus)
MSANIGVAIYQCADNFMTLYRADLEGYLKDMGYSVTIMDGKNDQNTQTEQINTFLQQGVDVLVINPVQTTSAQTIVDTVSPSGTPIVFINREPEESVLDSYKGKAAYVGADARQSGTYQGELILATDTQGDINGDGKITYIMAKGDPENIDAQYRTEYSIKALTDAGKEVECLYEYLDNWDQTTAQQDVANALSQYGEKIEVVFANNDAMALGALQSIQQAGRTVGKDVYLVGVDALVEAVQNVVDGNMTGTVLNDDVGQATKAAEATKLFVEGKDVEKYYWVDYVKVTKDNASQYLKEDGGSHHHHHH

SEQ ID NO: 279 - protein, 309 aa, synthetic construct
  REGION 1..309: note = fprGGBP_F16C (fprGGBP with signal sequence replaced with M; F16C, C8A, C105A, C106A, C143A, and C205A mutations; and GGSHHHHHH at C-terminus)
MSANIGVAIYQFADNCMTLYRADLEGYLKDMGYSVTIMDGKNDQNTQTEQINTFLQQGVDVLVINPVQTTSAQTIVDTVSPSGTPIVFINREPEESVLDSYKGKAAYVGADARQSGTYQGELILATDTQGDINGDGKITYIMAKGDPENIDAQYRTEYSIKALTDAGKEVECLYEYLDNWDQTTAQQDVANALSQYGEKIEVVFANNDAMALGALQSIQQAGRTVGKDVYLVGVDALVEAVQNVVDGNMTGTVLNDDVGQATKAAEATKLFVEGKDVEKYYWVDYVKVTKDNASQYLKEDGGSHHHHHH

SEQ ID NO: 280 - protein, 309 aa, synthetic construct
  REGION 1..309: note = fprGGBP_W180C (fprGGBP with signal sequence replaced with M; W180C, C8A, C105A, C106A, C143A, and C205A mutations; and GGSHHHHHH at C-terminus)
MSANIGVAIYQFADNFMTLYRADLEGYLKDMGYSVTIMDGKNDQNTQTEQINTFLQQGVDVLVINPVQTTSAQTIVDTVSPSGTPIVFINREPEESVLDSYKGKAAYVGADARQSGTYQGELILATDTQGDINGDGKITYIMAKGDPENIDAQYRTEYSIKALTDAGKEVECLYEYLDNCDQTTAQQDVANALSQYGEKIEVVFANNDAMALGALQSIQQAGRTVGKDVYLVGVDALVEAVQNVVDGNMTGTVLNDDVGQATKAAEATKLFVEGKDVEKYYWVDYVKVTKDNASQYLKEDGGSHHHHHH

SEQ ID NO: 281 - protein, 314 aa, synthetic construct
  REGION 1..314: note = cljGGBP_F11C (cljGGBP with signal sequence replaced with M; F11C, C77A, and C198A mutations; and GGSHHHHHH at C-terminus)
MPVIGFVAYECNNTWITELKNEIYKVSSGKARVDIWNGDNIQTVENDKINLFINRKVNVLDINPVDVNAAGQIIEKAKKANIPTVFVNRQPKKEDMEKWNKVYYVGAKAEQSGTIQGQMLVNYFKGHPTQDGTIRYIMLKGETRNQDAEKRTQYSIKALKDSGFKVQKVAEDTAMWDRTKAQEKMTSFISSYGPNFDAVIANNDDMALGAVDALKAAGYFNGGKYVPVVGVDATAPAVKAVEDGTLFGTVLNDAAKQGDAAFDLSYILSKGKIPDESNFKYKVTDGKYIWIDYKMITKENVQDAKGGSHHHHHH

SEQ ID NO: 282 - protein, 314 aa, synthetic construct
  REGION 1..314: note = cljGGBP_W15C (cljGGBP with signal sequence replaced with M; W15C, C77A, and C198A mutations; and GGSHHHHHH at C-terminus)
MPVIGFVAYEFNNTCITELKNEIYKVSSGKARVDIWNGDNIQTVENDKINLFINRKVNVLDINPVDVNAAGQIIEKAKKANIPTVFVNRQPKKEDMEKWNKVYYVGAKAEQSGTIQGQMLVNYFKGHPTQDGTIRYIMLKGETRNQDAEKRTQYSIKALKDSGFKVQKVAEDTAMWDRTKAQEKMTSFISSYGPNFDAVIANNDDMALGAVDALKAAGYFNGGKYVPVVGVDATAPAVKAVEDGTLFGTVLNDAAKQGDAAFDLSYILSKGKIPDESNFKYKVTDGKYIWIDYKMITKENVQDAKGGSHHHHHH

SEQ ID NO: 283 - protein, 314 aa, synthetic construct
  REGION 1..314: note = cljGGBP_W176C (cljGGBP with signal sequence replaced with M; W176C, C77A, and C198A mutations; and GGSHHHHHH at C-terminus)
MPVIGFVAYEFNNTWITELKNEIYKVSSGKARVDIWNGDNIQTVENDKINLFINRKVNVLDINPVDVNAAGQIIEKAKKANIPTVFVNRQPKKEDMEKWNKVYYVGAKAEQSGTIQGQMLVNYFKGHPTQDGTIRYIMLKGETRNQDAEKRTQYSIKALKDSGFKVQKVAEDTAMCDRTKAQEKMTSFISSYGPNFDAVIANNDDMALGAVDALKAAGYFNGGKYVPVVGVDATAPAVKAVEDGTLFGTVLNDAAKQGDAAFDLSYILSKGKIPDESNFKYKVTDGKYIWIDYKMITKENVQDAKGGSHHHHHH

SEQ ID NO: 284 - protein, 315 aa, synthetic construct
  REGION 1..315: note = cauGGBP_F12C (cauGGBP with signal sequence replaced with M; F12C, C78A, and C199A mutations; and GGSHHHHHH at C-terminus)
MEPVIGFVAYECNNTWITELKNEMYKVSNGKARVDIWNGNNIQTVENDKISLFINRKVDVLDINPVDVNAAGQIIEKAKKANIPTVFVNRQPKKEDVEKWNKVYYVGAKAEQSGTIQGQMLVNYFKGHPTQDGTIRYIMLKGEMRNQDAEKRTQYSIKALEDSGFKVQKVAEDTAMWDRTKAQEKMTSFISSYGPNFDAVIANNDDMALGAVDALKAAGYFNGGKYVPVVGVDATAPAVKAVEDGTLFGTVLNDAAKQGDAAFDLSYILSKGKIPDESNFKYKITDGKYIWIDYKMITKENVQDAKGGSHHHHHH

SEQ ID NO: 285 - protein, 315 aa, synthetic construct
  REGION 1..315: note = cauGGBP_W16C (cauGGBP with signal sequence replaced with M; W16C, C78A, and C199A mutations; and GGSHHHHHH at C-terminus)
MEPVIGFVAYEFNNTCITELKNEMYKVSNGKARVDIWNGNNIQTVENDKISLFINRKVDVLDINPVDVNAAGQIIEKAKKANIPTVFVNRQPKKEDVEKWNKVYYVGAKAEQSGTIQGQMLVNYFKGHPTQDGTIRYIMLKGEMRNQDAEKRTQYSIKALEDSGFKVQKVAEDTAMWDRTKAQEKMTSFISSYGPNFDAVIANNDDMALGAVDALKAAGYFNGGKYVPVVGVDATAPAVKAVEDGTLFGTVLNDAAKQGDAAFDLSYILSKGKIPDESNFKYKITDGKYIWIDYKMITKENVQDAKGGSHHHHHH

SEQ ID NO: 286 - protein, 315 aa, synthetic construct
  REGION 1..315: note = cauGGBP_W177C (cauGGBP with signal sequence replaced with M; W177C, C78A, and C199A mutations; and GGSHHHHHH at C-terminus)
MEPVIGFVAYEFNNTWITELKNEMYKVSNGKARVDIWNGNNIQTVENDKISLFINRKVDVLDINPVDVNAAGQIIEKAKKANIPTVFVNRQPKKEDVEKWNKVYYVGAKAEQSGTIQGQMLVNYFKGHPTQDGTIRYIMLKGEMRNQDAEKRTQYSIKALEDSGFKVQKVAEDTAMCDRTKAQEKMTSFISSYGPNFDAVIANNDDMALGAVDALKAAGYFNGGKYVPVVGVDATAPAVKAVEDGTLFGTVLNDAAKQGDAAFDLSYILSKGKIPDESNFKYKITDGKYIWIDYKMITKENVQDAKGGSHHHHHH

SEQ ID NO: 287 - protein, 336 aa, synthetic construct
  REGION 1..336: note = erhGGBP_F13C (erhGGBP with signal sequence replaced with M; F13C mutation; and GGSHHHHHH at C-terminus)
MKTYNIGVAIYKCDDNFMTLYREELASYFKEVGEKDGNTYKLDIQDGKQDQANQTEQINNFIAQGKDLIIANMVDPTAAGSIINSAKAKEIPVVFINREPETQELEIWPGKTTYVGADATQSGTIQGYMIANLENKGDIDGDGSVSYITLMGDPANVDAKQRTEYSVKGLEEKGVKTNALAQPYQANWDTAKGQEFTANALEQFGNKLEVVFANNDGMAVGAVTAIEAAGRKVGEDIFVVGVDAIPDAIELLKGGKLTGTVLNDHFNQSHTAVDVALELLQGKDVSAYYWHDYVGVTKPEEAELKRAEARKETVEEAVKRYAERDAQGGSHHHHHH

SEQ ID NO: 288 - protein, 336 aa, synthetic construct
  REGION 1..336: note = erhGGBP_F17C (erhGGBP with signal sequence replaced with M; F17C mutation; and GGSHHHHHH at C-terminus)
MKTYNIGVAIYKFDDNCMTLYREELASYFKEVGEKDGNTYKLDIQDGKQDQANQTEQINNFIAQGKDLIIANMVDPTAAGSIINSAKAKEIPVVFINREPETQELEIWPGKTTYVGADATQSGTIQGYMIANLENKGDIDGDGSVSYITLMGDPANVDAKQRTEYSVKGLEEKGVKTNALAQPYQANWDTAKGQEFTANALEQFGNKLEVVFANNDGMAVGAVTAIEAAGRKVGEDIFVVGVDAIPDAIELLKGGKLTGTVLNDHFNQSHTAVDVALELLQGKDVSAYYWHDYVGVTKPEEAELKRAEARKETVEEAVKRYAERDAQGGSHHHHHH

SEQ ID NO: 289 - protein, 336 aa, synthetic construct
  REGION 1..336: note = erhGGBP_W188C (erhGGBP with signal sequence replaced with M; W188C mutation; and GGSHHHHHH at C-terminus)
MKTYNIGVAIYKFDDNFMTLYREELASYFKEVGEKDGNTYKLDIQDGKQDQANQTEQINNFIAQGKDLIIANMVDPTAAGSIINSAKAKEIPVVFINREPETQELEIWPGKTTYVGADATQSGTIQGYMIANLENKGDIDGDGSVSYITLMGDPANVDAKQRTEYSVKGLEEKGVKTNALAQPYQANCDTAKGQEFTANALEQFGNKLEVVFANNDGMAVGAVTAIEAAGRKVGEDIFVVGVDAIPDAIELLKGGKLTGTVLNDHFNQSHTAVDVALELLQGKDVSAYYWHDYVGVTKPEEAELKRAEARKETVEEAVKRYAERDAQGGSHHHHHH

SEQ ID NO: 290 - protein, 325 aa, synthetic construct
  REGION 1..325: note = ereGGBP_F17C (ereGGBP with signal sequence replaced with M; F17C, C10A, C29A, C65A, C69A, and C183A mutations; and GGSHHHHHH at C-terminus)
MKQIYIGVTAYDQKDTCIGELIETFKKEAASLDTDKYDISMTIMDAAGSQRAQDDQVQEMIEDGANVLAINLADRTDLSHIINAAMEKDIPIIFFNREPVDEDLNRWDKLYYVGAKAKQSGQMQGELIADYIKNNPGVDKNGDGRIQYVILEGEMGHQDAIVRTESVTESMKNNGLQIEKLSAQIANWNRAQAQNRMTQLIGQYKNSIELVIANNDAMALGAIDAYEKLGVTESNVPAFFGVDGTDDGLEAVQQSKLAATVYNDKEGQAMAMAQLAYLAATGGSMKNIKFEDKKYVYLPYEKVTPDNVNEFVKDEQGGSHHHHHH

SEQ ID NO: 291 - protein, 325 aa, synthetic construct
  REGION 1..325: note = ereGGBP_Q13C (ereGGBP with signal sequence replaced with M; Q13C, C10A, C29A, C65A, C69A, and C183A mutations; and GGSHHHHHH at C-terminus)
MKQIYIGVTAYDCKDTFIGELIETFKKEAASLDTDKYDISMTIMDAAGSQRAQDDQVQEMIEDGANVLAINLADRTDLSHIINAAMEKDIPIIFFNREPVDEDLNRWDKLYYVGAKAKQSGQMQGELIADYIKNNPGVDKNGDGRIQYVILEGEMGHQDAIVRTESVTESMKNNGLQIEKLSAQIANWNRAQAQNRMTQLIGQYKNSIELVIANNDAMALGAIDAYEKLGVTESNVPAFFGVDGTDDGLEAVQQSKLAATVYNDKEGQAMAMAQLAYLAATGGSMKNIKFEDKKYVYLPYEKVTPDNVNEFVKDEQGGSHHHHHH

SEQ ID NO: 292 - protein, 325 aa, synthetic construct
  REGION 1..325: note = ereGGBP_W188C (ereGGBP with signal sequence replaced with M; W188C, C10A, C29A, C65A, C69A, and C183A mutations; and GGSHHHHHH at C-terminus)
MKQIYIGVTAYDQKDTFIGELIETFKKEAASLDTDKYDISMTIMDAAGSQRAQDDQVQEMIEDGANVLAINLADRTDLSHIINAAMEKDIPIIFFNREPVDEDLNRWDKLYYVGAKAKQSGQMQGELIADYIKNNPGVDKNGDGRIQYVILEGEMGHQDAIVRTESVTESMKNNGLQIEKLSAQIANCNRAQAQNRMTQLIGQYKNSIELVIANNDAMALGAIDAYEKLGVTESNVPAFFGVDGTDDGLEAVQQSKLAATVYNDKEGQAMAMAQLAYLAATGGSMKNIKFEDKKYVYLPYEKVTPDNVNEFVKDEQGGSHHHHHH

SEQ ID NO: 293 - protein, 18 aa, synthetic construct
  REGION 1..18: note = bZif Sequence
TGEKPYKCPECGKSFSRS

SEQ ID NO: 294 - protein, 32 aa, synthetic construct
  REGION 1..32: note = ZF-QNK
TGEKPYKCPECGKSFSRSDHLSRHQRTHQNKK

SEQ ID NO: 295 - protein, 6 aa, synthetic construct
  REGION 1..6: note = Hexahistidine Tag
HHHHHH

SEQ ID NO: 296 - protein, 6 aa, synthetic construct
  REGION 1..6: note = Hexalysine Tag
KKKKKK

SEQ ID NO: 297 - protein, 319 aa, synthetic construct
  REGION 1..319: note = ecGGBP.F16C (with signal peptide replaced with M; a F16C mutation; and a GGSHHHHHH at C-terminus)
MADTRIGVTIYKYDDNCMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSKKGGSHHHHHH

SEQ ID NO: 298 - protein, 312 aa, synthetic construct
  REGION 1..312: note = ttGGBP182C.2.0 (affinity-tuning mutant, 182C background (Table 6) with signal peptide replaced with M)
MKQLNIGVAIYKFDDTFMTGVRNAMTAEAQGKAKLNMVDSQNSQPTQNDQVDLFITKKMNALAINPVDRTAAGTIIDKAKQANIPVVFFNKEPLPEDMKKWDKVYYVGAKAEQSGILQGQIMADYWKAHPEADKNHDGVMQYVMLMGEPGHQDAILRTQYSIQTVKDAGIKVQELAKDYANCDRVTAHDKMAAWLSSFGDKIEAVFANNDDMALGAIEALKSAGYFTGNKYIPVVGVDATAPGIQAIKDGTLLGTVLNDAKNQAKATFNIAYELAQGITPTKDNIGYDITDGKYVWIPYKKITKDNISDAEQ

(19) United States
(12) Patent Application Publication
(10) Pub. No.: US 2024/0168032 A1
(43) Pub. Date: May 23, 2024

(54) GLUCOSE BIOSENSORS AND USES THEREOF

(71) Applicant: Duke University, Durham, NC (US)

(72) Inventors: Homme W. Hellinga, Durham, NC (US); Malin J. Allert, Raleigh, NC (US)

(21) Appl. No.: 18/340,009

(22) Filed: Jun. 22, 2023

Related U.S. Application Data
(63) Continuation of application No. 17/475,092, filed on Sep. 14, 2021, now abandoned.
Application No. 17/475,092 is a continuation of application No. 15/776,725, filed on May 16, 2018, now Pat. No. 11,156,615.
(60) Provisional application No. 62/257,784, filed on Nov. 20, 2015.
(60) Provisional application No. 62/257,796, filed on Nov. 20, 2015.

(51) Int. Cl. G01N 33/66; C07K 14/245; C12Q 1/54

(52) U.S. Cl. CPC G01N 33/66; C07K 14/245; C12Q 1/54; C07K 2319/20; G01N 2333/195; G01N 2333/245

(57) ABSTRACT

The present subject matter provides glucose biosensors as well as compositions, devices, and methods comprising such biosensors.

### RELATED APPLICATIONS

This application is a continuation of U.S. patent application Ser. No. 15/776,725, filed May 16, 2018, which is a national stage application, filed under 35 U.S.C. § 371, of International Application No. PCT/US2016/050297 filed Sep. 2, 2016, which claims benefit of priority to U.S. Provisional Application No. 62/257,796, filed Nov. 20, 2015, and U.S. Provisional Application No. 62/257,784, filed Nov. 20, 2015, the entire contents of each of which are incorporated herein by reference.

### INCORPORATION-BY-REFERENCE OF SEQUENCE LISTING

The contents of the text file named “35327-516001WO_Sequence_Listing.txt”, which was created on Sep. 2, 2016 and is 587 KB in size, is hereby incorporated by reference in its entirety.

### FIELD OF THE INVENTION

The present invention relates to compositions and methods for detecting and determining the concentration of glucose and/or galactose.

### BACKGROUND

Biosensors are analytical tools that can be used to measure the presence of a single molecular species in a complex mixture by combining the exquisite molecular recognition properties of biological macromolecules with signal transduction mechanisms that couple ligand binding to readily detectable physical changes (Hall, Biosensors, Prentice-Hall, Englewood Cliffs, N.J.; Scheller et al., Curr. Op. Biotech. 12:35-40, 2001). Ideally, a biosensor is reagentless and, in contrast to enzyme-based assays or competitive immunoassays, does not change composition as a consequence of making the measurement (Hellinga & Marvin, Trends Biotech. 16:183-189, 1998). Most biosensors combine a naturally occurring macromolecule such as an enzyme or an antibody, with the identification of a suitable physical signal particular to the molecule in question, and the construction of a detector specific to that system (Meadows, Adv. Drug Deliv. Rev. 21:177-189, 1996). Recently, molecular engineering techniques have been explored to develop macromolecules that combine a wide range of binding specificities and affinities with a common signal transduction mechanism, to construct a generic detection system for many different analytes (Hellinga & Marvin, Trends Biotech. 16:183-189, 1998).

Escherichia coli periplasmic binding proteins are members of a protein superfamily (bacterial periplasmic binding proteins, bPBPs) (Tam & Saier, Microbiol. Rev. 57:320-346, 1993). These proteins comprise two domains linked by a hinge region (Quiocho & Ledvina, Molec. Microbiol. 20:17-25, 1996). The ligand-binding site is located at the interface between the two domains. The proteins typically adopt two conformations: a ligand-free open form, and a ligand-bound closed form, which interconvert via a hinge-bending mechanism upon ligand binding. This global, ligand-mediated conformational change has been exploited to couple ligand binding to changes in fluorescence intensity by positioning single, environmentally sensitive fluorophores in locations that undergo local conformational changes in concert with the global change (Brune et al., Biochemistry 33:8262-8271, 1994; Gilardi et al., Prot. Eng. 10:479-486, 1997; Gilardi et al., Anal. Chem. 66:3840-3847, 1994; Marvin et al., Proc. Natl. Acad. Sci. USA 94:4366-4371, 1997, Marvin and Hellinga, J. Am. Chem. Soc. 120:7-11, 1998; Tolosa et al., Anal. Biochem. 267:114-120, 1999; Dattelbaum & Lakowicz, Anal. Biochem. 291:89-95, 2001; Marvin & Hellinga, Proc. Natl. Acad. Sci. USA 98:4955-4960, 2001; Salins et al., Anal. Biochem. 294:19-26, 2001).

### SUMMARY OF THE INVENTION

The invention provides improved biosensors that rapidly, reliably, and accurately detect and quantify glucose and/or galactose with significant advantages over previous systems. The present disclosure provides a biosensor for ligand, comprising a ligand-binding protein that is attached to a reporter group. The ligand may be glucose and/or galactose, and the ligand-binding protein includes a domain that binds the glucose and/or the galactose. The binding of a ligand to the ligand-binding domain of the ligand-binding protein causes a change in signaling by the reporter group. In various implementations, the biosensor may produce a signal when a ligand is bound to the ligand binding domain that is not produced (and/or that is different from a signal that is produced) when the ligand is absent from the ligand binding domain. These biosensors have widespread utility including in clinical, industrial, and environmental settings.

The glucose-binding proteins (biosensors) described herein are characterized by one conformational shape when bound to glucose and a different conformational shape when unbound to glucose, this change in shape affects the signal of a detectable label such as a fluorophore. The proteins are engineered to include a single cysteine to which the detectable label, e.g., a fluorophore is covalently attached. The biosensors are reagentless in that their monitoring mechanism requires neither additional substrates for a signal to develop, nor measurement of substrate consumption or product generation rates to determine glucose concentrations.

Among the advantages of these fluorophone-containing protein constructs is their high durability. The constructs retain their ability to bind glucose, change shape and thus detect the analyte, glucose, (a) even when immobilized (directly or indirectly) onto a solid surface such as a bead, plate, or sheet; (b) even after dessication (and subsequent reconstitution in a physiological buffer solution); (c) even when subjected to ambient conditions, e.g., conditions that can be encountered in storage and/or transportation; and (d) even when aged/stored for extended periods of time, e.g., weeks, months, or even years. Thus, the biosensors do not require refrigeration or a cold chain for distribution, permitting a wider range of applicability such as in-the-field use and reducing the cost of the sensor product.

For clinical applications, microliter volumes, e.g., 10 μl or less of a bodily fluid such as blood may be used. Moreover compared to conventional enzyme-based or antibody based assay systems, the results are achieved virtually instantaneously, e.g., within 30-60 seconds. A further advantage is that the sensors consistently and reliably bind to and detect the analyte (glucose) in complex fluids such as whole blood. Thus in a clinical setting, whole blood need not be processed, thereby reducing time and cost of the diagnostic procedure.

In non-clinical situations, e.g., industrial of commercial settings such as analysis of waste water, food or beverage production, or bioreactor/fermentation monitoring, the samples to be analyzed can be used directly upon sampling without further purification or processing, similarly reducing time and expense of the test. Moreover, the immobilized sensors need not be washed to remove unbound material following contacting the test sample with the sensors, because the unbound material (“contaminants”) do not materially affect the production of a precise, reliable detectable assay signal.

The glucose biosensors produce a dichromatic, ratiometric signal, i.e., the signal is defined as the quotient of the intensities at two independent wavelengths. The advantage of such a signal is that it provides an internally consistent reference. The self-calibrating nature of a ratiometric measurement removes the necessity for carrying out on-board calibration tests prior to each measurement.

Thus, reagentless, fluorescently responsive glucose sensors present a number of advantages over enzyme-based biosensors, including elimination of chemical transformations, elimination of substrate requirements, and self-calibration, which together lead to rapid response times, continuous monitoring capabilities, simple sample-handling, and lower cost due to simplified manufacturing and distribution processes.

### Ligand-Binding Proteins

Aspects of the present subject matter provide biosensors comprising a ligand-binding protein that binds glucose and/or galactose. Ligand binding proteins that bind both glucose and galactose may be referred to herein as “glucose-galactose binding proteins” (GGBPs). Typically, a natural GGBP has a glucose dissociation constant (KD) of about 10 μM or less at room temperature. However, GGBPs may be selected, designed, or engineered to detect different (e.g., higher or lower) levels of glucose and/or galactose. The ligand-binding protein may comprise a naturally occurring protein or a protein that is modified compared to a naturally occurring protein. For example, the ligand-binding protein may comprise one or more mutations compared to a naturally occurring protein. In some embodiments, the naturally occurring protein is a naturally occurring counterpart of the ligand-binding protein (e.g., the ligand-binding protein is a mutant of the naturally occurring counterpart).

A “naturally occurring counterpart” of a mutant polypeptide is a polypeptide produced in nature from which the mutant polypeptide has been or may be derived (e.g., by one or more mutations). For example, the naturally occurring counterpart is an endogenous polypeptide produced by an organism in nature, wherein the endogenous polypeptide typically does not have one or more of the mutations present in the mutant polypeptide. For convenience and depending on context, a naturally occurring counterpart may be referred to herein for the purpose of comparison and to illustrate the location and/or presence of one or more mutations, binding activities, and/or structural features.

As used herein, a “mutation” is a difference between the amino acid sequence of a modified polypeptide/protein and a naturally occurring counterpart. A polypeptide having a mutation may be referred to as a “mutant.” Non-limiting examples of mutations include insertions, deletions, and substitutions. However, the term “mutation” excludes (i) the addition of amino acids to the N-terminus or C-terminus of a polypeptide, and (ii) the omission/deletion/replacement of a polypeptide's signal peptide (e.g., replacement with another signal peptide or with a methionine).

The addition of amino acids to the N-terminus or C-terminus of a protein via a peptide bond may be referred to herein as a “fusion” of the amino acids to the protein. Similarly, an exogenous protein fused to amino acids (e.g., another protein, a fragment, a tag, or a polypeptide moiety) at its N-terminus or C-terminus may be referred to as a “fusion protein.” The added amino acids may comprise a heterologous polypeptide, e.g., a polypeptide reporter group such as a fluorescent protein, a moiety that facilitates the isolation or modification of a polypeptide, or a moiety that facilitates the attachment of a polypeptide to a substrate or surface. As used herein, “heterologous” when referring to the added amino acids (e.g., a “polypeptide”) of a fusion protein indicates that the polypeptide is not naturally part of the protein to which it is fused in the fusion protein. For example, the sequence of a heterologous polypeptide (“added amino acids”) that is fused to a protein is encoded by an organism other than the organism from which the protein is derived, is not known to be naturally encoded by any organism, or is encoded by a gene other than the wild-type gene that encodes an endogenous version of the protein.

As used herein the term “signal peptide” refers to a short (e.g., 5-30 or 10-60 amino acids long) stretch of amino acids at the N-terminus of a protein that directs the transport of the protein. In various embodiments, the signal peptide is cleaved off during the post-translational modification of a protein by a cell. Signal peptides may also be referred to as “targeting signals,” “leader sequences,” “signal sequences,” “transit peptides,” or “localization signals.” In instances where a signal peptide is not defined for a GGBP discussed herein, the signal peptide may optionally be considered to be, e.g., the first 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 5-15, 5-20, 5-25, 5-100, 10-15, 10-20, 10-25, 10-50, 10-100, 25-50, 25-75, or 25-100 amino acids from the N-terminus of the translated protein (compared to a protein that has not had the signal peptide removed, e.g., compared to a naturally occurring protein).

In some embodiments, the ligand-binding protein comprises 1, 2, 3, 4, 5, 6, 7, 8, 9, 10, 15, 20, 25, 30, 40, 50, 60, 70, 80, 90, 100, 1-10, 1-15, 1-20, 5-15, 5-20, 10-25, 10-50, 20-50, 25-75, 25-100 or more mutations compared to a naturally occurring protein while retaining at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 99.5%, or about 100% of the activity of the naturally occurring protein. Mutations include but are not limited to substitutions, insertions, and deletions. Non-limiting examples of ligand-binding proteins may have 1, 2, 3, 4, 5, 6, 7, 8, 9, 10, 15, 20, 25, 30, 40, 50, 60, 70, 80, 90, 100, 1-10, 1-15, 1-20, 5-15, 5-20, 10-25, 10-50, 20-50, 25-75, 25-100, or more substitution mutations compared to a naturally occurring protein while retaining at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 99.5%, or about 100% of the activity of the naturally occurring protein. In embodiments, at least one amino acid of the ligand-binding protein has been substituted with a cysteine. Alternatively or in addition, a ligand-binding protein may include one or more mutations that remove a cysteine, e.g., 1, 2, 3, 4, 5, 6, 7, 8, 9, 10 or more substitutions or deletions of a cysteine compared to a naturally occurring protein.

Alternatively, the ligand-binding protein is not a mutant. For example, a reporter group is fused to the N-terminus or the C-terminus of the ligand-binding protein.

In various embodiments, a ligand-binding protein may comprise a stretch of amino acids (e.g., the entire length of the ligand-binding protein or a portion comprising at least about 50, 100, 200, 250, 300, or 350 amino acids) in a sequence that is at least about 90%, 91%, 92%, 93%, 94%, 95%, 96%, 97%, 98%, 99%, 99.1%, 99.2%, 99.3%, 99.4%, or 99.5% identical to an amino acid sequence of a naturally occurring protein.

In some embodiments, the mutations are conservative, and the present subject matter includes many ligand-binding proteins in which the only mutations are substitution mutations. In non-limiting examples, a ligand-binding protein has no deletions or insertions compared to a naturally occurring protein (e.g., a naturally occurring counterpart). Alternatively, a ligand-binding protein may have (i) less than about 5, 4, 3, 2, or 1 inserted amino acids, and/or (ii) less than about 5, 4, 3, 2, or 1 deleted amino acids compared to a naturally occurring protein.

In various embodiments, a naturally occurring protein to which a ligand-binding protein is compared or has been derived (e.g., by mutation, fusion, or other modification) from a prokaryotic ligand-binding protein such as a bacterial ligand-binding protein. For example, the prokaryotic ligand-binding protein is a mutant, fragment, or variant of a natural (i.e., wild-type) bacterial protein. In various embodiments, the bacterial ligand-binding protein is from a thermophilic, mesophilic, or cryophilic prokaryotic microorganism (e.g., a thermophilic, mesophilic, or cryophilic bacterium).

A microorganism is “thermophilic” if it is capable of surviving, growing, and reproducing at temperatures between 41 and 140° ° C. (106 and 284 ºF), inclusive. In various embodiments, a thermophilic organism has an optimal growth temperature between 41 and 140° C., or that is at least about 45, 50, 55, 60, 65, 70, 75, 80, 85, 90, 95, 100, 105, 110, 115, 120, 125, 130, 135, or 140° C. Many thermophiles are archaea. Thermophilic eubacteria are suggested to have been among the earliest bacteria. Thermophiles are found in various geothermally heated regions of the Earth, such as hot springs and deep sea hydrothermal vents, as well as decaying plant matter, such as peat bogs and compost. Unlike other types of microorganisms, thermophiles can survive at much hotter temperatures, whereas other bacteria would be damaged and sometimes killed if exposed to the same temperatures. Thermophiles may be classified into three groups: (1) obligate thermophiles; (2) facultative thermophiles; and (3) hyperthermophiles. Obligate thermophiles (also called extreme thermophiles) require such high temperatures for growth, whereas facultative thermophiles (also called moderate thermophiles) can thrive at high temperatures, but also at lower temperatures (e.g. below 50° C.). Hyperthermophiles are particularly extreme thermophiles for which the optimal temperatures are above 80° C. Some microorganisms can live at temperatures higher than 100° C. at large depths in the ocean where water does not boil because of high pressure. Many hyperthermophiles are also able to withstand other environmental extremes such as high acidity or radiation levels. A compound (e.g., a protein or biosensor) is “thermotolerant” if it is capable of surviving exposure to temperatures above 41ºC. For example, in some embodiments a thermotolerant biosensor retains its function and does not become denatured when exposed to a temperature of about 45, 50, 55, 60, 65, 70, 75, 80, 85, 90, 95, 100, 105, 110, 115, 120, 125, 130, 135, or 140° C. for at least about 1, 2, 3, 4, 5, 6, 7, 8, 9, 10, 15, 20, 25, 30 or more minutes. In some embodiments, the thermotolerant compound survives exposure to 45, 50, 55, 60, 65, 70, 75, 80, 85, 90, 95, 100, 105, 110, 115, 120, 125, 130, 135, or 140° C. under pressure.

A microorganism is “mesophilic” if it is capable of surviving, growing, and reproducing at temperatures between 20 and 40° ° C. (68 and 104ºF), inclusive. “Psychrophiles” or “cryophiles” are microorganisms that are capable of growth and reproduction in cold temperatures. In various embodiments, a psychrophile is capable of growth and reproduction at a temperature of 10° C. or less, e.g., between −20° C. and +10° C.

In some embodiments, the microbial protein is produced by a bacterial microorganism, an archaean microorganism, an algal microorganism, a protozoan microorganism, or a fungal microorganism. In non-limiting examples, the microbial protein is produced by a Gram-positive bacterium or a Gram-negative bacterium. In various embodiments, a biosensor comprises a modified (e.g., mutated, fused, and/or conjugated) periplasmic binding protein or a cytoplasmic binding protein.

In non-limiting examples in which the ligand-binding protein is (1) an Escherichia coli (E. coli) glucose-galactose binding protein (GGBP) (e.g., has been derived from an E. coli GGBP via mutation) or (2) has an amino acid sequence that is at least 95%, 96, 97%, 98%, or 99% identical to the amino acid sequence of E. coli GGBP, the ligand-binding protein comprises a mutation other than a mutation of amino acid Y10, D14, N15, F16, N91, K92, E93, S112, S115, E149, H152, D154, A155, R158, M182, W183, N211, D236, L255, N256, D257, P294, or V296, wherein each amino acid position is numbered as in (SEQ ID NO: 17).

In certain embodiments, the ligand-binding protein is not derived from (e.g., by mutation, fusion, or other modification) an E. coli protein (such as ecGGBP) and does not comprise an amino acid sequence that is identical to the amino acid sequence of ecGGBP. For example, the naturally occurring counterpart of the ligand-binding protein is not an E. coli GGBP (e.g., the ligand-binding protein is not a mutant of, fusion protein comprising, or other variant of an E. coli GGBP). In some embodiments, the amino acid sequence of the ligand-binding protein is less than about 100%, 99%, 98%, 97%, 96%, 95%, 90%, 85%, 80%, 75%, 70%, 65%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, or 15% identical to an E. coli GGBP protein having amino acids in the sequence set forth as SEQ ID NO: 1, 16, or 17.

Aspects of the present subject matter provide a ligand-binding protein with a mutation that alters the interaction of the ligand-binding protein with a ligand (e.g., glucose and/or galactose). For example, the ligand-binding protein comprises a mutation that alters the interaction of the ligand-binding protein with the ligand compared to a naturally occurring counterpart. In some embodiments, the ligand-binding protein comprises a mutation that alters the interaction of an amino acid of the ligand-binding protein with a water molecule compared to a naturally occurring counterpart.

In some embodiments, the ligand-binding protein does not comprise a signal peptide. For example, the signal peptide (e.g., that is present in a naturally occurring counterpart) may be replaced with a methionine.

Exemplary implementations relate to a ligand such as glucose or galactose, wherein the ligand-binding protein comprises a GGBP. For example, the GGBP may comprise a mutant of, a fragment of, or a fusion protein comprising a microbial GGBP. In embodiments, the GGBP is not a mutant or fragment to which a heterologous polypeptide has been attached or added. In some embodiments, the ligand-binding protein has an affinity (KD) for glucose and/or galactose within the concentration range of glucose and/or galactose in a subject. In certain embodiments, the ligand-binding protein has an affinity (KD) for glucose in the range of about 0.2 mM to about 100 mM, about 0.1 mmol/L to about 120 mmol/L, or about 4 mmol/L to about 33 mmol/L. In various embodiments, the ligand-binding protein has an affinity (KD) for galactose in the range of about 0.8 mM to about 100 mM or about 0.2 mM to about 400 mM. The biosensor is capable of detecting glucose in, e.g. the hypoglycemic, euglycemic, hyperglycemic, or hyperglycemic-hyperosmotic range. Thus, unlike previous glucose sensors, the ratiometric reagentless glucose biosensors produce precise measurements over an extended glucose concentration range from hypoglycemic, euglycemic, hyperglycemic, as well as the hyperglycemic-hyperosmotic range in sample volumes of less than 10 μl. In some embodiments, the ligand-binding protein comprises a mutation that alters (e.g., increases or decreases) the interaction of the mutant with bound glucose compared to a naturally occurring protein (e.g., a microbial GGBP), wherein the interaction is with a portion of the glucose selected from the group consisting of 1-hydroxyl, 2-hydroxyl, 3-hydroxyl, 4-hydroxyl, 6-hydroxyl, pyranose ring, or any combination thereof. In non-limiting examples, the ligand-binding protein comprises a mutation that alters (e.g., increases or decreases) the mutant's affinity and/or specificity for glucose and/or galactose compared to a the unmutated ligand-binding protein (e.g., a microbial GGBP). In certain embodiments, the ligand-binding protein comprises a mutation that alters the interaction between the protein and bound glucose and/or galactose, a mutation that alters the equilibrium between the open and closed states of the ligand-binding protein, a mutation that alters the interaction between the ligand-binding protein and a reporter group (such as a fluorescent conjugate, e.g., the interaction with a carbonyl group or a naphthalene ring of a prodan-derived fluorophore such as Acrylodan or Badan), and/or a mutation that impacts indirect interactions that alter the geometry of the ligand binding site. In various embodiments, the mutation does not reduce, or negligibly impacts, the thermostability of the ligand-binding protein. In some embodiments, the mutation alters the thermostability of the ligand-binding protein by less than about 1, 2, 3, 4, 5, or 10° C.

The present subject matter provides a GGBP that is or is a mutant of: an Escherichia sp. (e.g., E. albertii, E. coli, E. fergusonii, E. hermannii, or E. vulneris) GGBP; a Thermoanaerobacter sp. (e.g., T. acetoethylicus, T. brockii, T. ethanolicus, T. italicus, T. kivui, T. mathranii, T. pseudethanolicus, T. siderophilus, T. sulfurigignens, T. sulfurophilus, T. thermocopriae, T. thermohydrosulfuricus, T. thermosaccharolyticum, T. uzonensis, or T. wiegelii) GGBP; a Clostridium sp. (e.g., C. absonum, C. aceticum, C. acetireducens, C. acetobutylicum, C. acidisoli, C. aciditolerans, C. acidurici, C. aerotolerans, C. aestuarii, C. akagii, C. aldenense, C. aldrichii, C. algidicarni, C. algidixylanolyticum, C. algifaecis, C. algoriphilum, C. alkalicellulosi, C. aminophilum, C. aminovalericum, C. amygdalinum, C. amylolyticum, C. arbusti, C. arcticum, C. argentinense, C. asparagiforme, C. aurantibutyricum, C. autoethanogenum, C. baratii, C. barkeri, C. bartlettii, C. beijerinckii, C. bifermentans, C. bolteae, C. bornimense, C. botulinum, C. bowmanii, C. bryantii, C. butyricum, C. cadaveris, C. caenicola, C. caminithermale, C. carboxidivorans, C. carnis, C. cavendishii, C. celatum, C. celerecrescens, C. cellobioparum, C. cellulofermentans, C. cellulolyticum, C. cellulosi, C. cellulovorans, C. chartatabidum, C. chauvoei, C. chromiireducens, C. citroniae, C. clariflavum, C. clostridioforme, C. coccoides, C. cochlearium, C. colletant, C. colicanis, C. colinum, C. collagenovorans, C. cylindrosporum, C. difficile, C. diolis, C. disporicum, C. drakei, C. durum, C. estertheticum, C. estertheticum estertheticum, C. estertheticum laramiense, C. fallax, C. felsineum, C. fervidum, C. fimetarium, C. formicaceticum, C. frigidicarnis, C. frigoris, C. ganghwense, C. gasigenes, C. ghonii, C. glycolicum, C. glycyrrhizinilyticum, C. grantii, C. haemolyticum, C. halophilum, C. hastiforme, C. hathewayi, C. herbivorans, C. hiranonis, C. histolyticum, C. homopropionicum, C. huakuii, C. hungatei, C. hydrogeniformans, C. hydroxybenzoicum, C. hylemonae, C. jejuense, C. indolis, C. innocuum, C. intestinale, C. irregulare, C. isatidis, C. josui, C. kluyveri, C. lactatifermentans, C. lacusfryxellense, C. laramiense, C. lavalense, C. lentocellum, C. lentoputrescens, C. leptum, C. limosum, C. litorale, C. lituseburense, C. ljungdahlii, C. lortetii, C. lundense, C. magnum, C. malenominatum, C. mangenotii, C. mayombei, C. methoxybenzovorans, C. methylpentosum, C. neopropionicum, C. nexile, C. nitrophenolicum, C. novyi, C. oceanicum, C. orbiscindens, C. oroticum, C. oxalicum, C. papyrosolvens, C. paradoxum, C. paraperfringens, C. paraputrificum, C. pascui, C. pasteurianum, C. peptidivorans, C. perenne, C. perfringens, C. pfennigii, C. phytofermentans, C. piliforme, C. polysaccharolyticum, C. populeti, C. propionicum, C. proteoclasticum, C. proteolyticum, C. psychrophilum, C. puniceum, C. purinilyticum, C. putrefaciens, C. putrificum, C. quercicolum, C. quinii, C. ramosum, C. rectum, C. roseum, C. saccharobutylicum, C. saccharogumia, C. saccharolyticum, C. saccharoperbutylacetonicum, C. sardiniense, C. sartagoforme, C. scatologenes, C. schirmacherense, C. scindens, C. septicum, C. sordellii, C. sphenoides, C. spiroforme, C. sporogenes, C. sporosphaeroides, C. stercorarium, C. stercorarium leptospartum, C. stercorarium stercorarium, C. stercorarium thermolacticum, C. sticklandii, C. straminisolvens, C. subterminale, C. sufflavum, C. sulfidigenes, C. symbiosum, C. tagluense, C. tepidiprofundi, C. termitidis, C. tertium, C. tetani, Clostridium tetanomorphum, C. thermaceticum, C. thermautotrophicum, C. thermoalcaliphilum, C. thermobutyricum, C. thermocellum, C. thermocopriae, C. thermohydrosulfuricum, C. thermolacticum, C. thermopalmarium, C. thermopapyrolyticum, C. thermosaccharolyticum, C. thermosuccinogenes, C. thermosulfurigenes, C. thiosulfatireducens, C. tyrobutyricum, C. uliginosum, C. ultunense, C. villosum, C. vincentii, C. viride, C. xylanolyticum, or C. xylanovorans) GGBP; a Salmonella sp. [e.g., S. bongori, S. enterica, S. enterica subspecies enterica, S. enterica subspecies salamae, S. enterica subspecies arizonae, S. enterica subspecies diarizonae, S. enterica subspecies houtenae, S. enterica subspecies indica, or S. enterica subspecies enterica serovar Typhimurium (S. typhimurium)] GGBP; a Caldicellulosiruptor sp. (e.g., C. saccharolyticus, C. acetigenus, C. bescii, C. changbaiensis, C. hydrothermalis, Caldicellulosiruptor hydrother, C. kristjanssonii, C. kronotskyensis, C. lactoaceticus, C. owensensis, or C. obsidiansis) GGBP; a Paenibacillus sp. (e.g., P. agarexedens, P. agaridevorans, P. alginolyticus, P. alkaliterrae, P. alvei, P. amylolyticus, P. anaericanus, P. antarcticus, P. assamensis, P. azoreducens, P. azotofixans, P. barcinonensis, P. borealis, P. brasilensis, P. brassicae, P. campinasensis, P. chinjuensis, P. chitinolyticus, P. chondroitinus, P. cineris, P. cookii, P. curdlanolyticus, P. daejeonensis, P. dendritiformis, P. durum, P. ehimensis, P. elgii, P. favisporus, P. glucanolyticus, P. glycanilyticus, P. gordonae, P. graminis, P. granivorans, P. hodogayensis, P. illinoisensis, P. jamilae, P. kobensis, P. koleovorans, P. koreensis, P. kribbensis, P. lactis, P. larvae, P. lautus, P. lentimorbus, P. macerans, P. macquariensis, P. massiliensis, P. mendelii, P. motobuensis, P. naphthalenovorans, P. nematophilus, P. odorifer, P. pabuli, P. peoriae, P. phoenicis, P. phyllosphaerae, P. polymyxa, P. popilliae, P. pulvifaciens, P. rhizosphaerae, P. sanguinis, P. stellifer, P. terrae, P. thiaminolyticus, P. timonensis, P. tylopili, P. turicensis, P. validus, P. vortex, P. vulneris, P. wynnii, P. xylanilyticus) GGBP; a Butyrivibrio sp. (e.g., B. proteoclasticus, B. crossotus, B. fibrisolvens, or B. hungatei) GGBP; a Roseburia sp. (e.g., R. intestinalis, R. faecis, R. hominis, or R. inulinivorans) GGBP; a Faecalibacterium sp. (e.g., F. prausnitzii) GGBP; an Erysipelothrix sp. (e.g., E. rhusiopathiae, E. inopinata, or E. tonsillarum) GGBP; or an Eubacterium sp. (e.g., E. rectale, E. acidaminophilum, E. nodatum, E. oxidoreducens, or E. foedans) GGBP.

In various embodiments, a biosensor comprises a GGBP that is or is a mutant of: an Escherichia coli GGBP (ecGGBP; SEQ ID NO: 1), a Thermoanaerobacter thermosaccharolyticum GGBP (ttGGBP; SEQ ID NO: 2), a Salmonella typhimurium GGBP (stGGBP; SEQ ID NO: 3), a Caldicellulosiruptor hydrothermalis GGBP (chyGGBP; SEQ ID NO: 4), a Caldicellulosiruptor obsidiansis GGBP (cobGGBP; SEQ ID NO: 5), a Paenibacillus sp. GGBP (pspGGBP; SEQ ID NO: 6); a Clostridium saccharolyticum GGBP (csaGGBP; SEQ ID NO: 7); a Clostridium autoethanogenum GGBP (cauGGBP; SEQ ID NO: 12); a Clostridium ljungdahlii GGBP (cljGGBP; SEQ ID NO: 11); a Butyrivibrio proteoclasticus GGBP (bprGGBP; SEQ ID NO: 8); a Roseburia intestinalis GGBP (rinGGBP_A; SEQ ID NO: 9 or rinGGBP_B; SEQ ID NO: 13); a Faecalibacterium prausnitzii GGBP (fprGGBP; SEQ ID NO: 10); a Erysipelothrix rhusiopathiae GGBP (erhGGBP; SEQ ID NO: 14); or a Eubacterium rectale GGBP (ereGGBP; SEQ ID NO: 15). In some embodiments, the GGBP comprises an amino acid sequence that is between 75% and 10% identical (e.g., between 25% and 50% identical) to the amino acid sequence of ecGGBP (SEQ ID NO: 1 or 17), ttGGBP (SEQ ID NO: 2 or 18), stGGBP (SEQ ID NO: 3 or 19), chyGGBP (SEQ ID NO: 4 or 20), pspGGBP (SEQ ID NO: 6 or 22); csaGGBP (SEQ ID NO: 7 or 23); bprGGBP (SEQ ID NO: 8 or 24); rinGGBP_A (SEQ ID NO: 9 or 25); rinGGBP_B (SEQ ID NO: 13 or 29); fprGGBP (SEQ ID NO: 10 or 26); cljGGBP (SEQ ID NO: 11 or 27); cauGGBP (SEQ ID NO: 12 or 28); erhGGBP (SEQ ID NO: 14 or 30); and/or ereGGBP (SEQ ID NO: 15 or 31).

Aspects of the present subject matter include a GGBP that is or is a mutant of a protein listed in Table 1, e.g., the protein numbered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138 in Table 1.

With regard to a defined polypeptide, % identity figures higher or lower than those provided herein will encompass various embodiments. Thus, where applicable, in light of a minimum % identity figure, a polypeptide may comprises an amino acid sequence which is at least 60%, 65%, 70%, 75%, 76%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to the reference SEQ ID NO or to each of the reference SEQ ID NOs. Where applicable, in light of a maximum % identity to a reference sequence, a polypeptide may comprise an amino acid sequence which is less than 75%, 70%, 65%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, or 15% identical to the reference SEQ ID NO or to each of the reference SEQ ID NOs. In certain embodiments, a polypeptide comprises amino acids in a sequence that is preferably at least about 10%, 11%, 12%, 13%, 14%, 15%, 16%, 17%, 18%, 19%, 20%, 21%, 22%, 23%, 24%, 25%, 26%, 27%, 28%, 29%, or 30% and less than about 75%, 70%, 65%, 60%, 55%, 50%, 45%, 44%, 43%, 42%, 41%, 40%, 39%, 38%, 37%, 36%, 35%, 34%, 33%, 32%, 31%, or 30% identical to the reference SEQ ID NO or to each of the reference SEQ ID NOs. Non-limiting examples of reference proteins and amino acid sequences disclosed herein include:
(i) a glucose-galactose binding protein from Escherichia coli (ecGGBP; genome, NC_002695; protein, WP_032329053, SEQ ID NO: 1);
(ii) a glucose-galactose binding protein from Thermoanaerobacterium thermosaccharolyticum (ttGGBP; genome, NC_014410; protein, YP_003852930.1, SEQ ID NO: 2);
(iii) a glucose-galactose binding protein from Salmonella typhimurium (stGGBP; genome, NC_003197; protein, WP_001036943, SEQ ID NO: 3);
(iv) a glucose-galactose binding protein from Caldicellulosiruptor hydrothermalis (chyGGBP; genome, NC_014652; protein identifier, YP_003991244.1, SEQ ID NO: 4);
(v) a glucose-galactose binding protein from Caldicellulosiruptor obsidiansis (cobGGBP; genome, NC_014392; protein, YP_003839461.1, SEQ ID NO: 5);
(vi) a glucose-galactose binding protein from Paenibacillus sp. (pspGGBP; genome, NC_013406; protein, YP_003243743.1, SEQ ID NO: 6);
(vii) a glucose-galactose binding protein from Clostridium saccharolyticum (csaGGBP; genome, NC_014376; protein, YP_003822565.1, SEQ ID NO: 7);
(viii) a glucose-galactose binding protein from Butyrivibrio proteoclasticus (bprGGBP; genome, NC_014387; protein, YP_003830205.1, SEQ ID NO: 8);
(ix) a glucose-galactose binding protein from Roseburia intestinalis (rinGGBP_A; genome, NC_021012; protein, YP_007778116.1, SEQ ID NO: 9);
(x) a glucose-galactose binding protein from Faecalibacterium prausnitzii (fprGGBP; genome, NC_021020; protein, YP_007799070.1, SEQ ID NO: 10);
(xi) a glucose-galactose binding protein from Clostridium ljungdahlii (cljGGBP; genome, NC_014328; protein, CLJU_c08950, SEQ ID NO: 11);
(xii) a glucose-galactose binding protein from Clostridium autoethanogenum (cauGGBP; genome, NC_022592; protein, CAETHG_2989, SEQ ID NO: 12);
(xiii) a glucose-galactose binding protein from Roseburia intestinalis (rinGGBP_B; genome, NC_021012; protein, YP_007778124.1, SEQ ID NO: 13);
(xiv) a glucose-galactose binding protein from Erysipelothrix rhusiopathiae (erhGGBP; genome, NC_015601; protein, YP_004561181.1, SEQ ID NO: 14); and
(xv) a glucose-galactose binding protein from Eubacterium rectale (ereGGBP; genome, NC_012781; protein, YP_002936409.1, SEQ ID NO: 15).

The GGBPs disclosed herein may optionally be fused (e.g., at their N-terminal and/or C-terminal ends) to a motif comprising a stretch of amino acids that facilitates the isolation or other manipulation such as conjugation to a moiety or immobilization on a substrate such as a plastic, a cellulose product such as paper, polymer, metal, noble metal, semi-conductor, or quantum dot (e.g., a fluorescent quantum dot). A non-limiting example of such a stretch of amino acids has the sequence: GGSHHHHHH (SEQ ID NO: 104). This motif is not required for and is not believed to influence or affect ligand-binding activity or signal transduction. For example, each of SEQ ID NOs: 32-103 (and the non-limiting examples of other proteins used in the experiments disclosed herein) comprises this motif (SEQ ID NO: 104). Alternatively or in addition, a ligand-binding protein may be fused to a heterologous polypeptide or “added amino acids” that facilitates the attachment thereof to a surface, such as the surface of a device.

In some embodiments, a polypeptide comprises 1, 2, 3, 4, 5, or more substitutions or deletions of a cysteine compared to the naturally occurring counterpart of the polypeptide (i.e., 1, 2, 3, 4, 5, or more native cysteines have been removed), e.g., 1, 2, 3, 4, 5, or more cysteine to alanine substitutions compared to the naturally occurring counterpart of the polypeptide. In some embodiments, all of cysteines of a polypeptide have been deleted and/or substituted compared to its natural counterpart.

In embodiments, the amino acid sequence of a protein comprises no more than 1, 2, 3, 4, 5, 6, 7, 8, 9, 10, 15, 20, or 25 mutations compared to its naturally occurring counterpart. In some embodiments, less than 25, 20, 15, 10, 9, 8, 7, 6, 5, 4, 3, or 2 of the mutations is a deletion or insertion of 1, 2, 3, 4, or 5 or no more than 1, 2, 3, 4, or 5 amino acids. In some embodiments, 1, 2, 3, 4, 5, 6, 7, 8, 9, 10, 15, 20, 25, or more of the mutations is a substitution mutation. In certain embodiments, every mutation to a protein compared to its naturally occurring counterpart is a substitution mutation. In various embodiments, 1, 2, 3, 4, 5, 6, 7, 8, 9, 10, 15, 20, 25 or more or all of the mutations to a protein compared to its naturally occurring counterpart is a conservative substitution mutation.

In various embodiments, a polypeptide does not have any insertion or deletion compared to its natural counterpart, other than (optionally) the removal of the signal peptide and/or the fusion of compounds such as another polypeptide at the N-terminus or C-terminus thereof.

Ligand-Binding Proteins Comprising a Primary Complementary Surface (PCS)

The following BLAST parameters are used to identify sequence homologues of GGBP: (1) Expect threshold is 10.0; (2) Gap cost is Existence: 11 and Extension: 1; (3) The Matrix employed is BLOSUM62; (4) The filter for low complexity regions is “on.” Such an alignment may be generated using the ProteinHunter program. The ProteinHunter package always executes BLAST searches, with the following command
“blastall -p blastp -m 8 -b 50000 -d % s -i <INPUT FILE> -o <OUTPUT FILE>”
where <INPUT FILE> and <OUTPUT FILE> specify the input and output files, respectively for a given calculation. This command executes the BLAST alignment program for protein sequences with default parameters, intrinsically set by the program. The BLAST program version is 2.2.24.

Sequence homologues of GGBP identified using BLAST may be aligned with ecGGBP using ClustalW to identify homologues that share a PCS with ecGGBP as discussed below.

Aspects of the present subject matter provide ligand-binding proteins that share a PCS with a GGBP disclosed herein. In embodiments, the PCS comprises at least about 5, 6, 7, 8, 9, or 10 amino acid positions used to identify a glucose-galactose binding protein. For example, the PCS of ecGGBP may comprise positions 14, 16, 91, 152, 154, 158, 183, 211, 236, and 256, wherein each position is counted as in SEQ ID NO: 17. In various embodiments, a protein shares a PCS with ecGGBP if the amino acid sequence of the protein has
(i) D or N at the position that aligns with position 14 of ecGGBP;
(ii) F, Y, or W at the position that aligns with position 16 of ecGGBP;
(iii) N or D at the position that aligns with position 91 of ecGGBP;
(iv) H, N, or Q at the position that aligns with position 152 of ecGGBP;
(v) D or N at the position that aligns with position 154 of ecGGBP;
(vi) R at the position that aligns with position 158 of ecGGBP;
(vii) W, F, or Y at the position that aligns with position 183 of ecGGBP; (vii)
(viii) N or D at the position that aligns with position 211 of ecGGBP;
(ix) D or N at the position that aligns with position 236 of ecGGBP; and
(x) N or D at the position that aligns with position 256 of ecGGBP,
   wherein the alignment between ecGGBP (SEQ ID NO: 17) and the protein is constructed using the ClustalW alignment program.

The ProteinHunter package always executes multiple sequence alignments with the following command
“clustalw -infile=<INPUT FILE> -outfile=<OUTPUTFILE> -align -quiet”

This command executes the CLUSTALW multi-sequence alignment program for protein sequences. There are no user-specified parameter settings that alter the alignment behavior of the program. The CLUSTALW program version is 2.1.

For convenience and depending on context, a position that aligns with a stated position of ecGGBP may be referred to herein as “equivalent” to the stated position.

Exemplary Ligand-Binding Proteins

Various biosensors provided herein comprise glucose-galactose binding proteins, such as glucose-galactose binding proteins that have altered amino acid sequences compared to their naturally occurring counterparts. In embodiments, such proteins are conjugated to reporter groups. ecGGBP is a non-limiting reference protein respect to glucose-galactose binding proteins. An alignment of ecGGBP with other polypeptides is provided in FIG. 3.

In various embodiments, a polypeptide of the present disclosure comprises
(a) an amino acid that is preferably (i) at least about 10%, 11%, 12%, 13%, 14%, 15%, 16%, 17%, 18%, 19%, 20%, 21%, 22%, 23%, 24%, 25%, 26%, 27%, 28%, 29%, or 30%, and (ii) less than about 75%, 70%, 65%, 60%, 55%, 50%, 45%, 44%, 43%, 42%, 41%, 40%, 39%, 38%, 37%, 36%, or 35% identical to ecGGBP;
(b) a cysteine substitution (compared to its naturally occurring counterpart) within a stretch of at least 5, 10, or 20 amino acids having at least about 50%, 55%, 60%, 65%, 75%, 80%, 85%, 90%, or 95% identity to a stretch of consecutive amino acids including position 183 of ecGGBP (e.g., a tryptophan to cysteine substitution);
(c) a stretch of at least 4, 5, 6, 7, 8, 9, 10, 11, 12, 13, 14, 15, 20, 25, or 30 amino acids having at least about 50%, 55%, 60%, 65%, 75%, 80%, 85%, 90%, or 95% identity to a stretch of consecutive amino acids including position 14 of ecGGBP;
(d) a stretch of amino acids in the sequence
   a. IYKX₁DDX₂FM (where X₁ is any amino acid, or where X₁ is Y, F, Q, or K; and where X₂ is any amino acid, or where X₂ is N or T) (SEQ ID NO: 182),
   b. YKX₁DDX₂FM (where X₁ is any amino acid, or where X₁ is Y, F, Q, or K; and where X₂ is any amino acid, or where X₂ is N or T) (SEQ ID NO: 183),
   c. YKX₁DDX₂F (where X₁ is any amino acid, or where X₁ is Y, F, Q, or K; and where X₂ is any amino acid, or where X₂ is N or T) (SEQ ID NO: 184),
   d. KX₁DDX₂F (where X₁ is any amino acid, or where X₁ is Y, F, Q, or K; and where X₂ is any amino acid, or where X₂ is N or T) (SEQ ID NO: 185),
   e. YKXDD (where X is any amino acid, or where X is Y, F, Q, or K) (SEQ ID NO: 186),
   f. KXDD (where X is any amino acid, or where X is Y, F, Q, or K) (SEQ ID NO: 187),
   g. DDXF (where X is any amino acid, or where X is N or T) (SEQ ID NO: 188),
   h. DX₁FMX₂X₃VR (where X₁ is any amino acid, or where X₁ is N or T; where X₂ is any amino acid, or where X₂ is S, T, G; and where X₃ is any amino acid, or where X₃ is V, G, E, or L) (SEQ ID NO: 189),
   i. DX₁FMX₂X₃V (where X₁ is any amino acid, or where X₁ is N or T; where X₂ is any amino acid, or where X₂ is S, T, G; and where X₃ is any amino acid, or where X₃ is V, G, E, or L) (SEQ ID NO: 190),
   j. DXFM (where X is any amino acid, or where X is N or T) (SEQ ID NO: 191),
   k. IYKX₁DNX₂FM (where X₁ is any amino acid, or where X₁ is Y, F, Q, or K; and where X₂ is any amino acid, or where X₂ is N or T) (SEQ ID NO: 192),
   l. YKX₁DNX₂FM (where X₁ is any amino acid, or where X₁ is Y, F, Q, or K; and where X₂ is any amino acid, or where X₂ is N or T) (SEQ ID NO: 193),
   m. YKX₁DNX₂F (where X₁ is any amino acid, or where X₁ is Y, F, Q, or K; and where X₂ is any amino acid, or where X₂ is N or T) (SEQ ID NO: 194),
   n. KX₁DNX₂F (where X₁ is any amino acid, or where X₁ is Y, F, Q, or K; and where X₂ is any amino acid, or where X₂ is N or T) (SEQ ID NO: 195),
   o. YKXDN (where X is any amino acid, or where X is Y, F, Q, or K) (SEQ ID NO: 196),
   p. KXDN (where X is any amino acid, or where X is Y, F, Q, or K) (SEQ ID NO: 197),
   q. DNXF (where X is any amino acid, or where X is N or T) (SEQ ID NO: 198),
   r. NX₁FMX₂X₃VR (where X₁ is any amino acid, or where X₁ is N or T; where X₂ is any amino acid, or where X₂ is S, T, G; and where X₃ is any amino acid, or where X₃ is V, G, E, or L) (SEQ ID NO: 199),
   s. NX₁FMX₂X₃V (where X₁ is any amino acid, or where X₁ is N or T; where X₂ is any amino acid, or where X₂ is S, T, G; and where X₃ is any amino acid, or where X₃ is V, G, E, or L) (SEQ ID NO: 200),
   t. NXFM (where X is any amino acid, or where X is N or T) (SEQ ID NO: 201);
(e) a stretch of amino acids in the sequence
   a. IYKX₁DDX₂FM (where X₁ is any amino acid, or where X₁ is Y, F, Q, or K; and where X₂ is any amino acid, or where X₂ is N or T) (SEQ ID NO: 182),
   b. YKX₁DDX₂FM (where X₁ is any amino acid, or where X₁ is Y, F, Q, or K; and where X₂ is any amino acid, or where X₂ is N or T) (SEQ ID NO: 183),
   c. YKX₁DDX₂F (where X₁ is any amino acid, or where X₁ is Y, F, Q, or K; and where X₂ is any amino acid, or where X₂ is N or T) (SEQ ID NO: 184),
   d. KX₁DDX₂F (where X₁ is any amino acid, or where X₁ is Y, F, Q, or K; and where X₂ is any amino acid, or where X₂ is N or T) (SEQ ID NO: 185),
   e. YKXDD (where X is any amino acid, or where X is Y, F, Q, or K) (SEQ ID NO: 186),
   f. KXDD (where X is any amino acid, or where X is Y, F, Q, or K) (SEQ ID NO: 187),
   g. DDXF (where X is any amino acid, or where X is N or T) (SEQ ID NO: 188),
   h. DX₁FMX₂X₃VR (where X₁ is any amino acid, or where X₁ is N or T; where X₂ is any amino acid, or where X₂ is S, T, G; and where X₃ is any amino acid, or where X₃ is V, G, E, or L) (SEQ ID NO: 189),
   i. DX₁FMX₂X₃V (where X₁ is any amino acid, or where X₁ is N or T; where X₂ is any amino acid, or where X₂ is S, T, G; and where X₃ is any amino acid, or where X₃ is V, G, E, or L) (SEQ ID NO: 190),
   j. DXFM (where X is any amino acid, or where X is N or T) (SEQ ID NO: 191),
   k. IYKX₁DNX₂FM (where X₁ is any amino acid, or where X₁ is Y, F, Q, or K; and where X₂ is any amino acid, or where X₂ is N or T) (SEQ ID NO: 192),
   l. YKX₁DNX₂FM (where X₁ is any amino acid, or where X₁ is Y, F, Q, or K; and where X₂ is any amino acid, or where X₂ is N or T) (SEQ ID NO: 193),
   m. YKX₁DNX₂F (where X₁ is any amino acid, or where X₁ is Y, F, Q, or K; and where X₂ is any amino acid, or where X₂ is N or T) (SEQ ID NO: 194),
   n. KX₁DNX₂F (where X₁ is any amino acid, or where X₁ is Y, F, Q, or K; and where X₂ is any amino acid, or where X₂ is N or T) (SEQ ID NO: 195),
   o. YKXDN (where X is any amino acid, or where X is Y, F, Q, or K) (SEQ ID NO: 196),
   p. KXDN (where X is any amino acid, or where X is Y, F, Q, or K) (SEQ ID NO: 197),
   q. DNXF (where X is any amino acid, or where X is N or T) (SEQ ID NO: 198),
   r. NX₁FMX₂X₃VR (where X₁ is any amino acid, or where X₁ is N or T; where X₂ is any amino acid, or where X₂ is S, T, G; and where X₃ is any amino acid, or where X₃ is V, G, E, or L) (SEQ ID NO: 199),
   S. NX₁FMX₂X₃V (where X₁ is any amino acid, or where X₁ is N or T; where X₂ is any amino acid, or where X₂ is S, T, G; and where X₃ is any amino acid, or where X₃ is V, G, E, or L) (SEQ ID NO: 200), or
   t. NXFM (where X is any amino acid, or where X is N or T) (SEQ ID NO: 201),
within about 50, 45, 40, 35, 20, or 15 amino acids of the N-terminus of the polypeptide, including or not including the amino acids of the signal peptide (also referred to herein as the leader peptide) of its natural counterpart;
(f) a stretch of at least 4, 5, 6, 7, 8, 9, 10, 11, 12, 13, 14, 15, 20, 25, or 30 amino acids having at least about 50%, 55%, 60%, 65%, 75%, 80%, 85%, 90%, or 95% identity to a stretch of consecutive amino acids including position 91 of ecGGBP
(g) a stretch of amino acids having the sequence
   a. PVVFFNKEP (SEQ ID NO: 202),
   b. PVVFXNKEP (where X is any amino acid, or where X is F, L, V, or I) (SEQ ID NO: 203),
   c. PVVFFNXEP (where X is any amino acid, or where X is K, R, E, or G) (SEQ ID NO: 204),
   d. PVVFX₁NX₂EP (where X₁ is any amino acid, or where X₁ is F, L, V, or I; and where X₂ is any amino acid, or where X₂ is K, R, E, or G) (SEQ ID NO: 205),
   e. VVFX₁NX₂EP (where X₁ is any amino acid, or where X₁ is F, L, V, or I; and where X₂ is any amino acid, or where X₂ is K, R, E, or G) (SEQ ID NO: 206),
   f. VFX₁NX₂EP (where X₁ is any amino acid, or where X₁ is F, L, V, or I; and where X₂ is any amino acid, or where X₂ is K, R, E, or G) (SEQ ID NO: 207),
   g. PVVFX₁NX₂E (where X₁ is any amino acid, or where X₁ is F, L, V, or I; and where X₂ is any amino acid, or where X₂ is K, R, E, or G) (SEQ ID NO: 208),
   h. PVVFXN (where X is any amino acid, or where X is F, L, V, or I) (SEQ ID NO: 209),
   i. PX₁VFX₂N (where X₁ is any amino acid, or where X₁ is V, I, L, or T; and where X₂ is any amino acid, or where X₂ is F, L, V, or I) (SEQ ID NO: 210),
   j. PVX₁FX₂N (where X₁ is any amino acid, or where X₁ is V, I, L, or T; and where X₂ is any amino acid, or where X₂ is F, L, V, or I) (SEQ ID NO: 211),
   k. FX₁NX₂EP (where X₁ is any amino acid, or where X₁ is F, L, V, or I; and where X₂ is any amino acid, or where X₂ is K, R, E, or G) (SEQ ID NO: 212), or
   l. FX₁NX₂E (where X₁ is any amino acid, or where X₁ is F, L, V, or I; and where X₂ is any amino acid, or where X₂ is K, R, E, or G) (SEQ ID NO: 213);
(h) a stretch of at least 4, 5, 6, 7, 8, 9, 10, 11, 12, 13, 14, 15, 20, 25, or 30 amino acids having at least about 50%, 55%, 60%, 65%, 75%, 80%, 85%, 90%, or 95% identity to a stretch of consecutive amino acids including position 150, 155, or 160 of ecGGBP;
(i) a stretch of amino acids having the sequence
   a. PGHPDAEART (SEQ ID NO: 214),
   b. PGHX₁DAX₂X₃RT (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 215),
   c. GHX₁DAX₂X₃RT (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 216),
   d. HX₁DAX₂X₃RT (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 217),
   e. DAX₁X₂RT (where X₁ is any amino acid, or where X₁ is E, I, K, or Q; and where X₂ is any amino acid, or where X₂ is A, L, V, K, Q, or Y) (SEQ ID NO: 218),
   f. PGHX₁DAX₂X₃R (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 219),
   g. PGHXDA (where X is any amino acid, or where X is P, Q, V, I, or E) (SEQ ID NO: 220),
   h. PGHXD (where X is any amino acid, or where X is P, Q, V, I, or E) (SEQ ID NO: 221),
   i. PGNPDAEART (SEQ ID NO: 222),
   j. PGNX₁DAX₂X₃RT (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 223),
   k. GNX₁DAX₂X₃RT (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 224),
   l. NX₁DAX₂X₃RT (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 225),
   m. PGNX₁DAX₂X₃R (where X₁ is any amino acid, or where X₁ is P, Q, V, I, or E; where X₂ is any amino acid, or where X₂ is E, I, K, or Q; and where X₃ is any amino acid, or where X₃ is A, L, V, K, Q, or Y) (SEQ ID NO: 226),
   n. PGNXDA (where X is any amino acid, or where X is P, Q, V, I, or E) (SEQ ID NO: 227), or
   o. PGNXD (where X is any amino acid, or where X is P, Q, V, I, or E) (SEQ ID NO: 228);
(j) a stretch of at least 4, 5, 6, 7, 8, 9, 10, 11, 12, 13, 14, 15, 20, 25, or 30 amino acids having at least about 50%, 55%, 60%, 65%, 75%, 80%, 85%, 90%, or 95% identity to a stretch of consecutive amino acids including position 183 of ecGGBP;
(k) a stretch of amino acids having the sequence
   a. DTAMWD (SEQ ID NO: 229),
   b. DTAMCD (SEQ ID NO: 230),
   c. DTAMW (SEQ ID NO: 231),
   d. DTAMC (SEQ ID NO: 232),
   e. TAMWD (SEQ ID NO: 233),
   f. TAMCD (SEQ ID NO: 234),
   g. AX₁WX₂X₃ (where X₁ is any amino acid, or where X₁ is M or N; where X₂ is any amino acid, or where X₂ is D or N; and where X₃ is any amino acid, or where X₃ is T or R) (SEQ ID NO: 235), or
   h. AX₁CX₂X₃ (where X₁ is any amino acid, or where X₁ is M or N; where X₂ is any amino acid, or where X₂ is D or N; and where X₃ is any amino acid, or where X₃ is T or R) (SEQ ID NO: 236);
(l) a stretch of at least 4, 5, 6, 7, 8, 9, 10, 11, 12, 13, 14, 15, 20, 25, or 30 amino acids having at least about 50%, 55%, 60%, 65%, 75%, 80%, 85%, 90%, or 95% identity to a stretch of consecutive amino acids including position 210 of ecGGBP;
(m) a stretch of amino acids having the sequence
   a. IEVVIANND (SEQ ID NO: 237),
   b. EVVIANND (SEQ ID NO: 238),
   c. IEVVIANN (SEQ ID NO: 239),
   d. EVVIANN (SEQ ID NO: 240),
   e. IEX₁VX₂X₃NND (where X₁ is any amino acid, or where X₁ is V, A, L, or C; where X₂ is any amino acid, or where X₂ is I, F, or L; and where X₃ is any amino acid, or where X₃ is A or C) (SEQ ID NO: 241),
   f. IEX₁VX₂X₃NN (where X₁ is any amino acid, or where X₁ is V, A, L, or C; where X₂ is any amino acid, or where X₂ is I, F, or L; and where X₃ is any amino acid, or where X₃ is A or C) (SEQ ID NO: 242),
   g. EX₁VX₂X₃NND (where X₁ is any amino acid, or where X₁ is V, A, L, or C; where X₂ is any amino acid, or where X₂ is I, F, or L; and where X₃ is any amino acid, or where X₃ is A or C) (SEQ ID NO: 243), or
   h. EX₁VX₂X₃NN (where X₁ is any amino acid, or where X₁ is V, A, L, or C; where X₂ is any amino acid, or where X₂ is I, F, or L; and where X₃ is any amino acid, or where X₃ is A or C) (SEQ ID NO: 244);
(n) a stretch of at least 4, 5, 6, 7, 8, 9, 10, 11, 12, 13, 14, 15, 20, 25, or 30 amino acids having at least about 50%, 55%, 60%, 65%, 75%, 80%, 85%, 90%, or 95% identity to a stretch of consecutive amino acids including position 235 or 240 of ecGGBP;
(o) a stretch of amino acids having the sequence
   a. PVFGVDA (SEQ ID NO: 245),
   b. VFGVDA (SEQ ID NO: 246),
   c. PVFGVD (SEQ ID NO: 247),
   d. FGVDA (SEQ ID NO: 248),
   e. PVXGVDA (where X is any amino acid or where X is F, V, or I) (SEQ ID NO: 249),
   f. VXGVDA (where X is any amino acid or where X is F, V, or I) (SEQ ID NO: 250),
   g. PVXGVD (where X is any amino acid or where X is F, V, or I) (SEQ ID NO: 251), or
   h. VXGVD (where X is any amino acid or where X is F, V, or I) (SEQ ID NO: 252);
(p) a stretch of at least 4, 5, 6, 7, 8, 9, 10, 11, 12, 13, 14, 15, 20, 25, or 30 amino acids having at least about 50%, 55%, 60%, 65%, 75%, 80%, 85%, 90%, or 95% identity to a stretch of consecutive amino acids including position 255 or 260 of ecGGBP
(q) a stretch of amino acids having the sequence GTVLNDA (SEQ ID NO: 253), GTVLND (SEQ ID NO: 254), GTVLN (SEQ ID NO: 255), or TVLND (SEQ ID NO: 256);
(r) no more than 1, 2, 3, 4, 5, 6, 7, 8, 9, 10, or 15 deleted or inserted amino acids compared to ecGGBP, not including added amino acids added to the N-terminus or C-terminus of the polypeptide compared to its natural counterpart, and including or not including the signal peptide of the natural counterpart of the polypeptide;
(s) at least 8, 9, 10, or 11, or exactly 8, 9, 10, or 11 α-helices; and/or
(t) at least 9, 10, 11, or 12 β-sheets or exactly 9, 10, 11, or 12 β-sheets.

In embodiments, two or more or each of features (c)-(q) above occurs in the polypeptide in the order listed above as the amino acid sequence of the polypeptide is viewed or read from the N-terminus to the C-terminus (with additional features and/or amino acid sequences therebetween). For example, the polypeptide may have an N-terminus, followed by feature (c), (d), or (e), followed by feature (f) or (g), followed by feature (h) or (i), followed by feature (j) or (k), followed by feature (l) or (m), followed by feature (n) or (o), followed by feature (p) or (q), followed by the C-terminus.

As used herein when referring to the order of features in an amino acid read from the N terminus to the C-terminus, a first feature is “followed by” a second feature when the second feature occurs after the first feature in the amino acid sequence. The words “followed by” do not require that the second feature immediately follow or be close to the first feature. For example, the N-terminus is followed by the C-terminus.

The features listed above are not limiting and may be combined with any other relevant features disclosed herein, including those listed below.

In some embodiments the polypeptide has the following sequence:

ZZZZ!GVXIY K%DDXFMXXV RXAXXXXXXX XXXX#XXZZD
XQNXQXXQN# X!DXXXXKXX XX$XINXVDX XAAGXXI#KA
XXXNXPVVFX NXEPXXXX$X XXDKXYYVGX XXX#SGXXXG
#XXXXXWXXX XXXDXNX#GX x#%V$xxG#P GHXDAxxRTX
%X!XXXXXXG IXXXXLXXDX AXWDXXXXXX KMXXXLXXZX
X#XIEXVXXN NDXMA$GA!E ALKXZZZZXX XXXXPVXGVD
AXXXXXXXXX XGX$XGTVLN DAXXQXKAXX XXAXXLXXGZ
XXXX#XXXXX IZ#XKX!X!X YXX!XKDNXX #ZZZZZZZZ

wherein each
Z is, individually, any amino acid or is absent,
X is, individually, any amino acid,
! is, individually, I or V,
$ is, individually, L or M,
% is, individually, F or Y, and
#is, individually, N, D, Q, or E.

In a non-limiting example, the glucose-galactose binding polypeptide comprises an N-terminal domain and a C-terminal domain connected by a flexible hinge, with the ligand-binding site (the ligand binding domain) located in the cleft between the N-terminal and the C-terminal domain.

In some embodiments, the glucose-galactose binding polypeptide comprises, from the N-terminus to the C-terminus, a first β-sheet (β1), followed by a first α-helix (α1), followed by a second β-sheet (β2), followed by a second α-helix (α2), followed by a third β-sheet (β3), followed by a third α-helix (α3), followed by a fourth β-sheet (β4), followed by a fourth α-helix (α4), followed by a fifth β-sheet (β5), followed by a first inter-domain hinge segment (h1), followed by a fifth α-helix (α5), followed by a sixth β-sheet (β6), followed by a sixth α-helix (α6), followed by a seventh β-sheet (β7), followed by a seventh α-helix (α7), followed by an eighth β-sheet (8), followed by an eighth α-helix (α8), followed by a ninth β-sheet (β9), followed by a ninth α-helix (α9), followed by a tenth β-sheet (β10), followed by a second inter-domain hinge segment (h2), followed by a tenth α-helix (α10), followed by a third inter-domain hinge segment (h3), followed by an eleventh β-sheet (β11). In some embodiments, the polypeptide comprises (i) 1, 2, or 3 amino acid substitutions between β1 and α1; (ii) 1, 2, or 3 amino acid substitutions between β2 and α2; (iii) 1, 2, or 3 amino acid substitutions between the β3 and α3; (iv) 1, 2, or 3 amino acid substitutions between the β4 and α4; (v) 1, 2, or 3 amino acid substitutions in β6; (vi) 1, 2, or 3 amino acid substitutions in α6; (vii) 1, 2, or 3 amino acid substitutions between the β7 and α7; (viii) 1, 2, or 3, amino acid substitutions in α8; and/or (ix) 1, 2, or 3 amino acid substitutions between the β9 and α9. In some embodiments, the substitutions are conservative substitutions. In various embodiments, the polypeptide comprises a cysteine substitution at β7, α7, or between β7 and α7.

The glucose-galactose binding polypeptide may further comprise 1, 2, or more Ca²⁺ binding sites.

In various embodiments, the Ca root-mean-square deviation (RMSD) between the backbone of the glucose-galactose binding polypeptide and ecGGBP, ttGGBP, stGGBP, chyGGBP, cobGGBP, pspGGBP, csaGGBP, bprGGBP, rinGGBP_A, rinGGBP_B, fprGGBP, cljGGBP, cauGGBP, erhGGBP, ereGGBP, and/or chyGGBP is, e.g., between about 0.1-3 Å, 0.5-1 Å, 0.5-1.5 Å, or 0.5-2 Å, or less than about 0.1 Å, 0.2 Å, 0.3 Å, 0.4 Å, 0.5 Å, 0.6 Å, 0.7 Å, 0.8 Å, 0.9 Å, 1.0 Å, 1.5 Å, 1.6 Å, 1.7 Å, 1.8 Å, 1.9 Å, 2.0 Å, 2.5 Å, or 3 Å. In some embodiments, the Ca RMSD between the N-terminal domain (i.e., the portion of the protein at the N-terminal side of the binding domain hinge) backbone of the glucose-galactose binding polypeptide and the corresponding domain of ecGGBP, ttGGBP, stGGBP, chyGGBP, cobGGBP, and/or pspGGBP is, e.g., between about 0.1-3 Å, 0.5-1 Å, 0.5-1.5 Å, or 0.5-2 Å, or less than about 0.1 Å, 0.2 Å, 0.3 Å, 0.4 Å, 0.5 Å, 0.6 Å, 0.7 Å, 0.8 Å, 0.9 Å, 1.0 Å, 1.5 Å, 1.6 Å, 1.7 Å, 1.8 Å, 1.9 Å, 2.0 Å, 2.5 Å, or 3 Å. In certain embodiments, the Ca RMSD between the C-terminal domain (i.e., the portion of the protein at the C-terminal side of the binding domain hinge) backbone of the glucose-galactose binding polypeptide and the corresponding domain of ecGGBP, ttGGBP, stGGBP, chyGGBP, cobGGBP, pspGGBP, csaGGBP, bprGGBP, rinGGBP_A, rinGGBP_B, fprGGBP, cljGGBP, cauGGBP, erhGGBP, ereGGBP, and/or chyGGBP is, e.g., between about 0.1-3 Å, 0.5-1 Å, 0.5-1.5 Å, or 0.5-2 Å, or less than about 0.1 Å, 0.2 Å, 0.3 Å, 0.4 Å, 0.5 Å, 0.6 Å, 0.7 Å, 0.8 Å, 0.9 Å, 1.0 Å, 1.5 Å, 1.6 Å, 1.7 Å, 1.8 Å, 1.9 Å, 2.0 Å, 2.5 Å, or 3 Å. Non-limiting considerations relating to the sequence and structural differences between homologous proteins are discussed in Chothia and Lesk (1986) The EMBO Journal, 5(4):823-826, the entire content of which is incorporated herein by reference.

Non-limiting examples of glucose-galactose binding polypeptides that are useful in biosensors provided herein include ecGGBP, ttGGBP, stGGBP, chyGGBP, cobGGBP, pspGGBP, csaGGBP, bprGGBP, rinGGBP_A, rinGGBP_B, fprGGBP, cljGGBP, cauGGBP, erhGGBP, ereGGBP, and chyGGBP. In embodiments, a biosensor comprises a modified ecGGBP, ttGGBP, stGGBP, chyGGBP, cobGGBP, pspGGBP, csaGGBP, bprGGBP, rinGGBP_A, rinGGBP_B, fprGGBP, cljGGBP, cauGGBP, erhGGBP, ereGGBP, or chyGGBP polypeptide having an amino acid substitution compared to its naturally occurring counterpart, such that the polypeptide has a cysteine at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 270, 271, 272, 273, 274, 275, 276, 277, 278, 279, 280, 281, 282, 283, 284, 285, 286, 287, 288, 289, 290, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, or 383 or any combination of 1, 2, 3, 4, or 5 thereof, wherein the position corresponds a SEQ ID NO disclosed herein for ecGGBP, ttGGBP, stGGBP, chyGGBP, cobGGBP, pspGGBP, csaGGBP, bprGGBP, rinGGBP_A, rinGGBP_B, fprGGBP, cljGGBP, cauGGBP, erhGGBP, ereGGBP, or chyGGBP. In embodiments, the cysteine is conjugated to a reporter group.

In embodiments, a biosensor comprises a modified ecGGBP. In non-limiting examples, the modified ecGGBP may comprise one or more, or any combination of the following substitutions compared to its naturally occurring counterpart: Y10X, D14X, N15X, F16X, P70X, N91X, K92X, S112X, S115X, E149X, H152X, P153X, D154X, A155X, R158X, M182X, W183X, N211X, D212X, D236X, L238X, D257X, P294X, and V296X, where X is any amino acid, an amino acid that results in a conservative substitution, or a cysteine, and where each position is counted in ecGGBP without including the signal peptide (SEQ ID NO: 17). In some embodiments, the modified ecGGBP comprises 1, 2, 3, 4, 5, 6, 7, 8, 9, 10, or 11 of the following substitutions: Y10A, Y10C, D14C, D14A, D14Q, D14N, D14S, D14T, D14E, D14H, D14L, D14Y, D14F, F16L, F16A, F16C, N91C, N91A, K92A, K92C, E149C, E149K, E149Q, E149S, H152C, H152A, H152F, H152Q, H152N, D154C, D154A, D154N, A155C, A155S, A155H, A155L, A155F, A155Y, A155N, A155K, A155M, A155W, A155Q, R158C, R158A, R158K, M182C, M182W, W183C, N211C, N211F, N211W, N211K, N211Q, N211S, N211H, N211M, N211C, D212C, L238C, D236C, D236A, D236N, N256A, N256D, D257C, P294C, and V293C.

In various embodiments, a biosensor comprises a modified ttGGBP. In non-limiting examples, the modified ttGGBP may comprise one or more, or any combination of the following substitutions compared to its naturally occurring counterpart: Y11X, D15X, T16X, F17X, G20X, N42X, V67X, R69X, R91X, E92X, A111X, Q148X, H151X, Q152X, A154X, N181X, W182X, D183X, D211X, T237X, T240X, L257X, N258X, D259X, A260X, and K300X where X is any amino acid, an amino acid that results in a conservative substitution, or a cysteine, and where each position is counted in ttGGBP with the signal peptide replaced with a methionine (SEQ ID NO: 18). In some embodiments, the modified ttGGBP comprises 1, 2, 3, 4, 5, 6, 7, 8, 9, 10, or 11 of the following substitutions: Y11C, D15A, D15E, D15N, D15C, T16S, T16N, T16C, F17C, G20A, G20C, N42C, V67C, R69P, R69C, R91K, E92C, A111C, Q148S, Q148K, Q148E, Q148C, H151Q, H151N, H151F, H151C, Q152P, Q152C, A154S, A154N, A154M, A154F, A154C, N181C, W182C, D183C, D211A, D211C, T237C, T240A, L257C, N258D, N258S, N258A, N258C, D259C, A260N, A260Q, A260R, A260K, A260W, A260F, A260Y, A260S, A260C, and K300C.

In embodiments, a biosensor comprises a modified stGGBP. In non-limiting examples, the modified stGGBP may comprise one or more, or any combination of the following substitutions compared to its naturally occurring counterpart: Y11X, Y13X, D15X, N16X, F17X, P71X, N92X, K93X, P113X, S116X, E150X, H153X, P154X, D155X, A156X, R159X, M183X, W184X, N211X, N212X, D213X, A214X, D237X, L239X, D258X, P295X, and V297X where X is any amino acid, an amino acid that results in a conservative substitution, or a cysteine, and where each position is counted in stGGBP with the signal peptide replaced with a methionine (SEQ ID NO: 19). In some embodiments, the modified stGGBP comprises 1, 2 or 3 of the following mutations: Y13C, F17C, and W184C.

In embodiments, a biosensor comprises a modified chyGGBP. In non-limiting examples, the modified chyGGBP may comprise one or more, or any combination of the following substitutions compared to its naturally occurring counterpart: F12X, D14X, T15X, F16X, R68X, N89X, R90X, A110X, S113X, E147X, H150X, Q151X, D152X, A153X, R156X, M180X, W181X, N207X, N208X, D209X, D210X, D237X, T239X, D258X, V296X, and Y298X where X is any amino acid, an amino acid that results in a conservative substitution, or a cysteine, and where each position is counted in chyGGBP with the signal peptide replaced with a methionine (SEQ ID NO: 20). In some embodiments, the modified chyGGBP comprises 1, 2, or 3 of the following mutations: F12C, F16C, C39A, W181C, and C206A.

In embodiments, a biosensor comprises a modified cobGGBP. In non-limiting examples, the modified cobGGBP may comprise one or more, or any combination of the following substitutions compared to its naturally occurring counterpart: F12X, D14X, T15X, F16X, C39X, R68X, N89X, R90X, A110X, S113X, E147X, H150X, Q151X, D152X, A153X, R156X, C173X, M180X, W181X, C206X, N207X, N208X, D209X, D210X, D237X, T239X, D258X, P297X, and Q299X where X is any amino acid, an amino acid that results in a conservative substitution, or a cysteine, and where each position is counted in cobGGBP with the signal peptide replaced with a methionine (SEQ ID NO: 21). In some embodiments, the modified cobGGBP comprises 1, 2, or 3 of the following mutations: F12C, F16C, C39A, C173A, W181C, and C206A.

In embodiments, a biosensor comprises a modified pspGGBP. In non-limiting examples, the modified pspGGBP may comprise one or more, or any combination of the following substitutions compared to its naturally occurring counterpart: F9X, D11X, T12X, F13X, R65X, N86X, R87X, A107X, S110X, E144X, H147X, Q148X, D149X, A150X, R153X, M177X, W178X, N204X, N205X, D206X, D207X, D234X, T236X, 255X, A294X, and K296X where X is any amino acid, an amino acid that results in a conservative substitution, or a cysteine, and where each position is counted in pspGGBP with the signal peptide replaced with a methionine (SEQ ID NO: 22). In some embodiments, the modified pspGGBP comprises 1, 2, or 3 of the following mutations: F9C, F13C, and W178C.

In embodiments, a biosensor comprises a modified csaGGBP. In non-limiting examples, the modified csaGGBP may comprise one or more, or any combination of the following substitutions compared to its naturally occurring counterpart: Y14X, D16X, F18X, C62X, I72X, C82X, N93X, R94X, C113A, S118X, A121X, E152X, N155X, E156X, D157X, S158X, R161X, N185X, W186X, C211X, D241X, L243X, D262X, D290X, I292X, I297X, F299X, Q301X, and T302X where X is any amino acid, an amino acid that results in a conservative substitution, or a cysteine, and where each position is counted in csaGGBP with the signal peptide replaced with a methionine (SEQ ID NO: 23). In some embodiments, the modified csaGGBP comprises 1, 2, 3, 4, 5, 6, 7, or 8 of the following mutations: Y14C, F18C, C62A, C82A, C113A, W186C, and C211A.

In embodiments, a biosensor comprises a modified bprGGBP. In non-limiting examples, the modified bprGGBP may comprise one or more, or any combination of the following substitutions compared to its naturally occurring counterpart: C8X, K12X, D14X, N15X, F16X, S72X, N93X, R94X, C112X, C116X, A118X, S121X, A153X, N156X, 1157X, D158X, A159X, C179X, N186X, W187X, C211X, N212X, N213X, D214X, A215X, D241X, D243X, K251X, C289X, D290X, and V292X where X is any amino acid, an amino acid that results in a conservative substitution, or a cysteine, and where each position is counted in bprGGBP with the signal peptide replaced with a methionine (SEQ ID NO: 24). In some embodiments, the modified bprGGBP comprises 1, 2, 3, 4, 5, 6, 7, 8, or 9 of the following mutations: C8A, K12C, F16C, C112A, C116A, C179A, W187C, C211A, and C289A.

In embodiments, a biosensor comprises a modified rinGGBP_A. In non-limiting examples, the modified rinGGBP_A may comprise one or more, or any combination of the following substitutions compared to its naturally occurring counterpart: C6X, F10X, D12X, N13X, F14X, S70X, N91X, R92X, C114X, A116X, Q118X, D151X, N154X, V155X, D156X, A157X, R160X, C177X, N184X, W185X, C210X, N211X, N212X, D213X, A214X, D240X, L242X, L250X, C288X, D289X, and V291X where X is any amino acid, an amino acid that results in a conservative substitution, or a cysteine, and where each position is counted in rinGGBP_A with the signal peptide replaced with a methionine (SEQ ID NO: 25). In some embodiments, the modified rinGGBP_A comprises 1, 2, 3, 4, 5, 6, 7, or 8 of the following mutations: C6A, F10C, F14C, C114A, C177A, W185C, C210A, and C288A.

In embodiments, a biosensor comprises a modified rinGGBP_B. In non-limiting examples, the modified rinGGBP_B may comprise one or more, or any combination of the following substitutions compared to its naturally occurring counterpart: Q13X, D15X, T16X, F17X, C66X, C70A, R76X, N97X, R98X, A118X, S121X, E155X, H158X, Q159X, D160X, A161X, R164X, N188X, W189X, N215X, N216X, D217X, D218X, D244X, T246X, D265X, P301X, A303X, and C306X where X is any amino acid, an amino acid that results in a conservative substitution, or a cysteine, and where each position is counted in rinGGBP_B with the signal peptide replaced with a methionine (SEQ ID NO: 29). In some embodiments, the modified rinGGBP_B comprises 1, 2, 3, 4, 5, or 6 of the following mutations: Q13C, F17C, C66A, C70A, W189C, and C306A.

In embodiments, a biosensor comprises a modified fprGGBP. In non-limiting examples, the modified fprGGBP may comprise one or more, or any combination of the following substitutions compared to its naturally occurring counterpart: C8A, F12X, D14X, N15X, F16X, T69X, N90X, R91X, C105X, C106X, A113X, S116X, C143X, D146X, N149X, 1150X, D151X, A152X, R155X, N179X, W180X, C205A, N206X, N207X, D208X, A209X, D235X, L237X, N243X, D284X, and V286X where X is any amino acid, an amino acid that results in a conservative substitution, or a cysteine, and where each position is counted in fprGGBP with the signal peptide replaced with a methionine (SEQ ID NO: 26). In some embodiments, the modified fprGGBP comprises 1, 2, 3, 4, 5, 6, or 7 of the following mutations: C8A, F12C, F16C, C105A, C106A, C143A, W180C, and C205A.

In embodiments, a biosensor comprises a modified cljGGBP. In non-limiting examples, the modified cljGGBP may comprise one or more, or any combination of the following substitutions compared to its naturally occurring counterpart: F11X, N13X, T14X, W15X, V67X, C77X, N88X, R89X, A109X, S112X, E142X, N145X, Q146X, D147X, A148X, R151X, M175X, W176X, C198X, N201X, N202X, D203X, D204X, D231X, T233X, D252X, D291X, and K294X where X is any amino acid, an amino acid that results in a conservative substitution, or a cysteine, and where each position is counted in cljGGBP with the signal peptide replaced with a methionine (SEQ ID NO: 27). In some embodiments, the modified cljGGBP comprises 1, 2, 3, 4, or 5 of the following mutations: F11C, W15C, C77A, W176C, and C198A.

In embodiments, a biosensor comprises a modified cauGGBP. In non-limiting examples, the modified cauGGBP may comprise one or more, or any combination of the following substitutions compared to its naturally occurring counterpart: F12X, N14X, T15X, W16X, V68X, C78X, N89X, R90X, A110X, S113X, E143X, N146X, Q147X, D148X, A149X, R152X, M176X, W177X, C199X, N203X, N204X, D205X, D206X, D233X, T235X, D254X, D293X, and K295X where X is any amino acid, an amino acid that results in a conservative substitution, or a cysteine, and where each position is counted in cauGGBP with the signal peptide replaced with a methionine (SEQ ID NO: 28). In some embodiments, the modified cauGGBP comprises 1, 2, 3, 4, or 5 of the following mutations: F12C, W16C, C78A, W177C, and C199A.

In embodiments, a biosensor comprises a modified erhGGBP. In non-limiting examples, the modified erhGGBP may comprise one or more, or any combination of the following substitutions compared to its naturally occurring counterpart: F13X, D15X, N16X, F17X, P76X, N97X, R98X, A119X, S122X, D153X, N156X, V157X, D158X, A159X, R162X, M187X, W188X, N214X, N215X, D216X, G217X, D243X, 1245X, D264X, E312X, and V314X where X is any amino acid, an amino acid that results in a conservative substitution, or a cysteine, and where each position is counted in erhGGBP with the signal peptide replaced with a methionine (SEQ ID NO: 30). In some embodiments, the modified erhGGBP comprises 1, 2, or 3 of the following mutations: F13C, F17C, and W188C.

In embodiments, a biosensor comprises a modified ereGGBP. In non-limiting examples, the modified ereGGBP may comprise one or more, or any combination of the following substitutions compared to its naturally occurring counterpart: Q13X, D15X, T16X, F17X, C29X, C65X, C69X, R75X, N96X, R97, A117X, S120X, E154X, H157X, Q158X, D159X, A160X, R163X, C183X, N187X, W188X, N214X, N215X, D216X, A217X, D243X, T245X, D264X, P301X, and E303X where X is any amino acid, an amino acid that results in a conservative substitution, or a cysteine, and where each position is counted in ereGGBP with the signal peptide replaced with a methionine (SEQ ID NO: 31). In some embodiments, the modified ereGGBP comprises 1, 2, 3, 4, 5, 6, 7, or 8 of the following mutations: C10A, Q13C, F17C, C29A, C65A, C69A, C183A, and W188C.

In various embodiments, the mutant glucose-galactose binding polypeptide's glucose and/or galactose disassociation constant differs by at least about 1 μM, 5 μM, 10 μM, 20 μM, 25 μM, 30 μM, 35 μM, 40 μM, 45 μM, 50 μM, 75 μM, 100 M, 200 μM, 300 μM, 400 μM, 500 μM, 600 μM, 700 μM, 800 M, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, or 100 mM (increase or decrease) compared to its naturally occurring counterpart.

The biosensors and ligand-binding proteins provided herein are robust and useful at a wide range of physical conditions, e.g., pressure, temperature, salinity, osmolality, and pH conditions. For example, biosensors and ligand-binding proteins provided herein may survive substantial periods of time after being dried or exposed to high temperatures. In some embodiments, the biosensor maintains at least about 75%, 80%, 85%, 90%, 95%, 96%, 97%, 98%, 99%, 99.5%, 99.9%, or more of its signal transduction activity after exposure to a temperature of about 40, 45, 50, 55, 60, 65, 70, 75, 80, 85, 90, 95, 100, 105, 110, 115, 120, or 125, or 40-125° C. for about 1, 2, 3, 4, 5, 6, 15, 30, 60, 120, 180, 240, or 360 minutes. In certain embodiments, the biosensor maintains at least about 75%, 80%, 85%, 90%, 95%, 96%, 97%, 98%, 99%, 99.5%, 99.9%, or more of its signal transduction activity after 1, 2, 3, 4, or 5 freeze-thaw cycles in an aqueous solution. In various embodiments, the biosensor maintains at least about 75%, 80%, 85%, 90%, 95%, 96%, 97%, 98%, 99%, 99.5%, 99.9%, or more of its signal transduction activity after storage at a temperature of between 20-37° C. for about 1, 2, 3, 4, 5, 6, 7, 8, 9, 10, 11, 12, 18, 24, or 1-24 months in dry form. In some embodiments, the optimal functional temperature of the biosensor is between 41 and 122° C., between 20 and 40° C., or less than about 10° C. (e.g., between -20 and +10° C.). Devices, compositions, and biosensors provided herein may be stored, e.g., with or without protection from exposure to light. In some embodiments, the devices, compositions, and biosensors are stored in the dark, e.g., with protection from light.

### Reporter Group Attachment

Aspects of the present subject matter provide a biosensor that comprises a one or more reporter groups attached to a ligand-binding protein, wherein binding of a ligand to a ligand-binding domain of the ligand-binding protein causes a change in signaling by the reporter group. In various embodiments, the reporter group is attached to an endosteric site, an allosteric site, or a peristeric site of the ligand-binding protein. In embodiments, the reporter group is covalently or noncovalently attached to the ligand-binding protein.

For convenience and depending on context, a reporter group may be referred to by a name of an unattached form of the reporter group regardless of whether the reporter group is attached to a ligand-binding protein. For example, a compound known as “Compound A” when in an unconjugated form may be referred to herein as “Compound A” when in a form that is attached to a ligand-binding protein. In a specific example, the term “Acrylodan” is used to refer to unreacted/unconjugated Acrylodan, as well as Acrylodan that is conjugated to a ligand-binding protein.

In certain embodiments, a biosensor comprises a reporter group that is conjugated to a ligand-binding protein, and the reporter group is conjugated to an amino acid of the protein that is at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 4, 6, 8, 10, 11, 12, 13, 14, 15, 16, 17, 18, 19, 20, 25, 30, 35, 40, 45, 50, 55, 60, 65, 70, 75, 80, 85, 90, 95, or 100 angstroms (Å) from the ligand when the ligand is bound to the protein. In some embodiments, the reporter group is conjugated to an amino acid of the protein that is within an α-helix or a β-sheet. In some embodiments, the reporter group is conjugated to an amino acid that (i) is not within an α-helix or a β-sheet, but is within about 10, 9, 8, 7, 6, 5, 4, 3, 2, or 1 amino acids of an amino acid of the protein's amino acid sequence that is within α-helix or β-sheet. In some embodiments, the reporter group is conjugated to an amino acid that is in an inter-domain hinge amino acid region between two domains of a protein. In some embodiments, the reporter group is conjugated to an amino acid that is in an inter-domain hinge amino acid region between (i) α-helix and a β-sheet; (ii) two α-helixes; or (iii) two β-sheets of a protein. In some embodiments, the reporter group is conjugated to an amino acid (e.g., a cysteine such as a cysteine added by substitution compared to a naturally corresponding polypeptide) between positions 1-25, 25-50, 50-75, 75-100, 100-125, 125-150, 150-175, 175-200, 200-225, 225-250, 250-275, 275-350, 275-300, 275-325, 300-325, 300-350, 300-383, or 350-383 (inclusive) of a polypeptide (e.g., not including N-terminal fusion proteins compared to the polypeptide's naturally occurring counterpart).

Direct signaling relationships between proteins and reporter groups are readily designed by replacing a residue known to form a ligand contact with a cysteine to which the fluorophore is attached (“endosteric” attachment site). Other, indirect signaling relationships can be established in two ways. The first relies on visual inspection of the ligand complex structure, and identifying residues that are located in the vicinity of the binding site, but do not interact directly with the ligand, and that are likely to be involved in conformational changes. Typically, such “peristeric” sites are located adjacent to the residues that form direct contacts with the bound ligand. In the case of the bPBPs, such residues are located at the perimeter of the inter-domain cleft that forms the ligand binding site. The environment of these peristeric sites changes significantly upon formation of the closed state. These are examples of positions which are proximal to the ligand-binding pocket/domain. The second, most general, approach identifies sites in the protein structure that are located anywhere in the protein, including locations at some distance away from the ligand-binding site (i.e., distal to the ligand-binding pocket/domain), and undergo a local conformational change in concert with ligand binding. If the structures of both the open and closed states are known, then such “allosteric” sites can be identified using a computational method that analyzes the conformational changes that accompany ligand binding (Marvin et al., Proc. Natl. Acad. Sci. USA 94:4366-4371, 1997). Alternatively, once allosteric sites have been identified in one bPBP, modeling and structural homology arguments can be invoked to identify such sites in other bPBPs in which only one state has been characterized (Marvin & Hellinga, J. Am. Chem. Soc. 120:7-11, 1998). This generalized conformational analysis also may identify peristeric and endosteric sites, which were identified and classified by visual inspection.

In non-limiting implementations, the reporter group is attached to said ligand-binding protein via a biotin-avidin interaction. The reporter group may be, e.g., conjugated to biotin and the ligand-binding protein is conjugated to avidin. In an example, the avidin is bound to four biotin molecules wherein each biotin molecule is individually conjugated to a reporter group. Alternatively, the reporter group is conjugated to avidin and the ligand-binding protein is conjugated to biotin. For example, the avidin is bound to four biotin molecules, wherein each biotin molecule is individually conjugated to a ligand-binding protein.

As used herein, “conjugated” means covalently attached. One compound may be directly conjugated to another compound, or indirectly conjugated, e.g., via a linker.

In some embodiments, the reporter group is directly attached to said ligand-binding protein. In various embodiments, the reporter group is attached to an amino acid of the ligand-binding protein that is at least about 2, 4, 6, 8, 10, 11, 12, 13, 14, 15, 16, 17, 18, 19, 20, 25, 30, 35, 40, 45, 50, 55, 60, 65, 70, 75, 80, 85, 90, 95, or 100 angstroms (Å) from the ligand when the ligand is bound to the ligand-binding protein. In certain embodiments, the reporter group is conjugated to an amino acid having a position within positions 1-25, 25-50, 50-75, 75-100, 100-125, 125-150, 150-175, 175-200, 200-225, 225-250, 250-275, or 275-300 of the ligand-binding protein, wherein position 1 is the N-terminal amino acid of the ligand-binding protein. In non-limiting examples, the reporter group is conjugated to an amino acid of the ligand-binding protein that is (a) within an α-helix or a -sheet of the ligand-binding protein; (b) not within an α-helix; (c) not within a β-sheet; (d) within about 5 or 10 amino acids of an amino acid that is within an α-helix or β-sheet; (e) within a stretch of consecutive amino acids that links two domains of the ligand-binding protein; (f) within a stretch of consecutive amino acids that links an α-helix and a β-sheet; (g) within a stretch of consecutive amino acids that links two α-helices; or (h) within a stretch of consecutive amino acids that links two β-sheets. In some embodiments, the reporter group is directly attached to the N-terminus or the C-terminus of the ligand-binding protein.

The reporter group may be conjugated to the ligand-binding protein a variety of linkers or bonds, including (but not limited to) a disulfide bond, an ester bond, a thioester bond, an amide bond, or a bond that has been formed by a click reaction. In some embodiments, the click reaction is a reaction between (a) an azide and an alkyne; (b) an azide and an alkyne in the presence of Cu(I); (c) an azide and a strained cyclooctyne; (d) an azide and a dibenzylcyclooctyne, a difluorooctyne, or a biarylazacyclooctynone; (e) a diaryl-strained-cyclooctyne and a 1,3-nitrone; (f) an azide, a tetrazine, or a tetrazole and a strained alkene; (g) an azide, a tetrazine, or a tretrazole and a oxanorbornadiene, a cyclooctene, or a trans-cycloalkene; (h) a tetrazole and an alkene; or (i) a tetrazole with an amino or styryl group that is activated by ultraviolet light and an alkene. These exemplary click chemistry reactions have high specificity, efficient kinetics, and occur in vivo under physiological conditions. See, e.g., Baskin et al. Proc. Natl. Acad. Sci. USA 104(2007):16793; Oneto et al. Acta biomaterilia (2014); Neves et al. Bioconjugate chemistry 24(2013):934; Koo et al. Angewandte Chemie 51(2012):11836; Rossin et al. Angewandte Chemie 49(2010):3375, and U.S. Patent Application Publication No. 20160220686, published Aug. 4, 2016, the entire content of each of which is incorporated herein by reference. For a review of a wide variety of click chemistry reactions and their methodologies, see e.g., Nwe K and Brechbiel M W, 2009 Cancer Biotherapy and Radiopharmaceuticals, 24(3): 289-302; Kolb H C et al., 2001 Angew. Chem. Int. Ed. 40: 2004-2021. The entire contents of each of the foregoing references are incorporated herein by reference.

As used herein, the term “linker” refers to a molecule or sequence (such as an amino acid sequence), that attaches, as in a bridge, one molecule or sequence to another molecule or sequence. “Linked” means attached or bound by covalent bonds, or non-covalent bonds, or other bonds, such as van der Waals forces. In some embodiments, a linker comprises a chemical structure that has resulted from a reaction used to attach one molecule to another.

In various implementations of the present subject matter, the reporter group is conjugated to a cysteine of the ligand-binding protein. The cysteine may be present on a natural counterpart or version of the ligand-binding protein or added to the ligand-binding protein by a substitution mutation. In some embodiments, the cysteine is at the N-terminus or the C-terminus of said ligand-binding protein.

Non-limiting examples relate to the conjugation of a reporter group to a primary amine of the ligand-binding protein. In certain embodiments, the primary amine is present in a lysine of said ligand-binding protein. The lysine may be present on a natural counterpart or version of the ligand-binding protein or added to the ligand-binding protein by a substitution mutation. In various embodiments, the lysine is at the N-terminus or the C-terminus of the ligand-binding protein.

Aspects of the present subject matter provide a biosensor in which the reporter group is attached to the ligand-binding protein via a linker. In some embodiments, the linker comprises an organic compound that is less than about 30, 20, 15, or 10 Å long. Non-limiting examples of spacers include O, S, NH, PH, and alkyl spacers.

“Alkyl,” as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), more preferably 20 or fewer carbon atoms, more preferably 12 or fewer carbon atoms, and most preferably 8 or fewer carbon atoms. Likewise, preferred cycloalkyls have from 3-10 carbon atoms in their ring structure, and more preferably have 5, 6 or 7 carbons in the ring structure. The ranges provided above are inclusive of all values between the minimum value and the maximum value. The term “alkyl” includes both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety. Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. The alkyl groups may also contain one or more heteroatoms within the carbon backbone. Preferably the heteroatoms incorporated into the carbon backbone are oxygen, nitrogen, sulfur, and combinations thereof. In certain embodiments, the alkyl group contains between one and four heteroatoms.

In some embodiments, the linker comprises a bond formed by a chemical reaction involving a reactive group such as a maleimide group. Alternatively or in addition, the linker comprises a stretch of amino acids. In a non-limiting example, the linker comprises a polyglycine linker. In embodiments, the polyglycine linker comprises 2, 3, 4, 5, or more glycines. Optionally, the polyglycine linker further comprises a serine.

In various implementations, the reporter group is attached to a linker via a covalent bond and the linker is attached to a ligand-binding protein via a covalent bond. In embodiments, the covalent bond between the linker and the reporter group and/or the covalent bond between the linker and the ligand-binding protein is a disulfide bond, an ester bond, a thioester bond, an amide bond, a carbamate bond, or a bond that has been formed by a click reaction. Non-limiting examples of click reactions include reactions between an azide and an alkyne; an azide and an alkyne in the presence of Cu(I); an azide and a strained cyclooctyne; an azide and a dibenzylcyclooctyne, a difluorooctyne, or a biarylazacyclooctynone; a diaryl-strained-cyclooctyne and a 1,3-nitrone; an azide, a tetrazine, or a tetrazole and a strained alkene; an azide, a tetrazine, or a tretrazole and a oxanorbornadiene, a cyclooctene, or a trans-cycloalkene; a tetrazole and an alkene; or a tetrazole with an amino or styryl group that is activated by ultraviolet light and an alkene.

### Reporter Groups

Various types of reporter groups may be used in embodiments of the present subject matter. For example, the reporter group may comprise a fluorophore that produces a fluorescent signal. Biosensors comprising a fluorophore may be referred to herein as fluorescently responsive sensors (FRSs).

Preferably, the binding of ligand to an FRS results in a change in ratiometric ΔR in the signal from a reporter group. A ratiometric signal (R_{1,2}) is defined as the quotient of two intensities, I_{λ1} and I_{λ2}, measured at two independent wavelengths, λ₁ and λ₂ and may be calculated according to the following equation:

R_{1,2}=I_{λ1}/I_{λ2}

In some embodiments, intensities are integrated over a range of wavelengths in a recorded emission spectrum. In some embodiments, intensities are integrated over 10-nm, 15-nm, 20-nm, 25-nm, 30-nm, 35-nm, 40-nm, 45-nm, 50-nm, 75-nm, 100-nm, 10-40-nm, 10-50-nm, 20-50-nm, or 10-100-nm regions, centered between 400-800 nm, e.g. between 420 nm and 520 nm for λ₁, and 400-800 nm, e.g. between 500 nm to 600 nm for λ₂. In some embodiments, intensities are recorded through a bandpass filter. A non-limiting example of a bandpass filter is a 10-nm, 15-nm, 20-nm, 25-nm, 30-nm, 35-nm, 40-nm, 45-nm, 50-nm, 75-nm, 100-nm, 10-40-nm, 10-50-nm, 20-50-nm, or 10-100-nm bandpass filter, centered between 400-800 nm, e.g. at 452 nm for λ₁ and at 400-800 nm, e.g. at 528 nm (λ₂).

Aspects of the present subject matter provide FRSs whose emission spectra change (e.g., the shape of the emission spectra change) in response to ligand binding. In various embodiments, the ratio of intensities at two chosen wavelengths of an FRS's emission spectrum changes upon ligand binding. In some embodiments, the emission wavelength and/or intensity of the fluorophore changes when the position of atoms within the fluorophore changes with respect to each other (e.g., due to the rotation of bound atoms with respect to each other or a change in the angle of a bond). In non-limiting examples, the emission wavelength and/or intensity of the fluorophore changes when (i) one portion of the fluorophore rotates around a bond axis compared to another portion of the fluorophore and/or (ii) when the angle of a bond between two atoms of said fluorophore changes. In a non-limiting example, the fluorophore is a prodan-derived fluorophore (e.g., Acrylodan or Badan) and binding of ligand alters the orientation of a dimethylamino group, a naphthalene ring, and/or a carbonyl with respect to the ligand-binding protein and/or each other. In a non-limiting example, the degree of polarization of a dipole on the fluorophore changes in response to ligand binding. In various embodiments, the emission wavelength and/or intensity of the fluorophore changes when an atom electrostatically interacts with said fluorophore. For example, the emission wavelength and/or intensity of the fluorophore changes when the source of a positive or negative charge changes its distance with respect to the fluorophore within about 1, 2, 3, 4, 5, or 10 Å of the fluorophore. In some embodiments, the fluorophore exhibits hypsochromicity or bathochromicity upon ligand binding to the ligand-binding domain of the ligand-binding protein. In certain embodiments, the fluorophore has an emission wavelength comprising a wavelength of about 550 nanometers (nm), 575 nm, 600 nm, 625 nm, 650 nm, or 550-650 nm.

In some embodiments, the signal comprises the emission intensity of the fluorophore recorded at a single wavelength or range of wavelengths. The change in signal may be a shift in the single wavelength or range of wavelengths. In some embodiments, the shift in the wavelength is at least about 1 nm, at least about 2 nm, at least about 3 nm, at least about 4 nm, at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, at least about 100 nm, at least about 105 nm, at least about 110 nm, at least about 115 nm, at least about 120 nm, at least about 125 nm, or at least about 130 nm. In some embodiments, the shift in the wavelength is about 1 nm to about 20 nm, about 2 nm to about 20 nm, about 3 nm to about 20 nm, about 4 nm to about 20 nm, about 5 nm to about 20 nm, about 1 nm to about 19 nm, about 1 nm to about 18 nm, about 1 nm to about 17 nm, 1 nm to about 16 nm, about 1 nm to about 15 nm, about 1 nm to about 14 nm, about 1 nm to about 13 nm, about 1 nm to about 12 nm, about 1 nm to about 11 nm, or about 1 nm to about 10 nm. In some embodiments, the shift in the wavelength is about 1 nm to about 20 nm. In some embodiments, the shift in the wavelength is about 1 nm to about 130 nm.

In certain embodiments, the signal comprises the ratio or quotient of the emission intensities recorded at two distinct wavelengths or ranges of wavelengths, i.e., a ratiometric signal. For example, as shown in FIGS. 1A-C, ligand binding may be determined by measuring the ratio of blue to green emission intensities. The change in signal may be decreased emission intensity at one wavelength, and no change in emission intensity at the other wavelength. The change in signal may be increased emission intensity at one wavelength, and no change in emission intensity at the other wavelength. The change in signal may be increased emission intensity at one wavelength, and increased emission intensity at the other wavelength. The change in signal may be decreased emission intensity at one wavelength, and decreased emission intensity at the other wavelength. The change in signal may be increased emission intensity at one wavelength, and decreased emission intensity at the other wavelength. In some embodiments, the change in ratio of the emission intensities recorded at two distinct wavelengths or ranges of wavelengths may be at least about 1.1-fold, at least about 1.2-fold, at least about 1.4-fold, at least about 1.6-fold, at least about 1.8-fold, at least about 2.0-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 12-fold, at least about 14-fold, at least about 16-fold, at least about 18-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, or at least about 100-fold. In some embodiments, the change in ratio of the emission intensities recorded at two distinct wavelengths or ranges of wavelengths may be a decrease of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, or of 5-25%, 25-50%, 25-75%, 50-75%, 50-90%, or 75-99% or the reciprocal thereof.

The change in signal may be a change in the ratio of the two distinct wavelengths or ranges of wavelengths. The change in signal may be a shift in the two distinct wavelengths or ranges of wavelengths. In some embodiments, one wavelength shifts. In some embodiments, both wavelengths shift. In some embodiments, the shift in the wavelength is at least about 1 nm, at least about 2 nm, at least about 3 nm, at least about 4 nm, at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, at least about 100 nm, at least about 105 nm, at least about 110 nm, at least about 115 nm, at least about 120 nm, at least about 125 nm, or at least about 130 nm. In some embodiments, the shift in the wavelength is about 1 nm to about 20 nm, about 2 nm to about 20 nm, about 3 nm to about 20 nm, about 4 nm to about 20 nm, about 5 nm to about 20 nm, about 1 nm to about 19 nm, about 1 nm to about 18 nm, about 1 nm to about 17 nm, 1 nm to about 16 nm, about 1 nm to about 15 nm, about 1 nm to about 14 nm, about 1 nm to about 13 nm, about 1 nm to about 12 nm, about 1 nm to about 11 nm, or about 1 nm to about 10 nm. In some embodiments, the shift in the wavelength is about 1 nm to about 20 nm. In some embodiments, the shift in the wavelength is about 1 nm to about 130 nm.

A fluorophore may comprise, e.g., a fluorescent protein or an organic compound having a molecular weight less than about 2000 Daltons (Da). Non-limiting examples of commercially available fluorophores include such as 5-iodoacetamidofluorescein (5IAF) or 6-iodoacetamidofluorescein (6IAF), rhodamine, Oregon Green, eosin, Texas Red, indocarbocyanine, oxacarbocyanine, thiacarbocyanine, merocyanine, Badan, Acrylodan, IAEDANS, comprising 3-cyano-7-hydroxycoumarin, 7-hydroxycoumarin-3-carboxylic acid, 6,8-difluoro-7-hydroxy-4-methylcoumarin, or 7-amino-4-methylcoumarin, pyridyloxazole, nitrobenzoxadiazole, benzoxadiazole, DRAQ5, DRAQ7, or CyTRAK Orange, cascade blue, Nile red, Nile blue, cresyl violet, oxazine 170, proflavin, acridine orange, acridine yellow, auramine, crystal violet, malachite green, porphin, phthalocyanine, bilirubin, pyrene, N,N′-dimethyl-N-(iodoacetyl)-N′-(7-nitrobenz-2-ox-a-1,3-diazol-4-yl)ethylenediamide (NBD), N-((2-(iodoacetoxy)ethyl)-N-methy-l)amino-7-nitrobenz-2-oxa-1,3-diazole (NBDE), Acrylodan, JPW4039, JPW4042, JPW4045, Oregon Green, Pacific Blue, CPM, N,N′-Dimethyl-N-(Iodoacetyl)-N′-(7-Nitrobenz-2-Oxa-1,3-Diazol-4-yl)Ethylenediamine (IANBD), 7-diethylamino-3-(4′-maleimidylphenyl)-4-methylcoumarin (CPM), BODIPY 499, BODIPY 507/545, BODIPY 499/508, Alexa 432, Alexa488, Alexa532, Alexa546, Cy5, or 1-(2-maleimidylethyl)-4-(5-(4-methoxyphenyl)oxazol-2-yl)pyridinium methanesulfonate (PyMPO maleimide) (PyMPO). In various embodiments, the reporter group was thiol-reactive prior to being conjugated to a polypeptide disclosed herein. In embodiments, the reporter group is linked to a polypeptide disclosed herein via a disulfide bond. Additional non-limiting examples of commercially available fluorophores include fluorescent proteins such as Blue Fluorescent Protein (BFP), TagBFP, mTagBFP2, Azurite, Enhanced Blue Florescent Protein 2 (EBFP2), mKalama1, Sirius, Sapphire, T-Sapphire, Cyan Fluorescent Protein (CFP); Enhanced Cyan Fluorescent Protein (ECFP), Cerulean, SCFP3A, mTurquoise, mTurquoise2, monomeric Midoriishi-Cyan, TagCFP, mTFP1, AmCyan1, Green Fluorescent Protein (GFP), Enhanced Green Fluorescent Protein (EGFP), Emerald, Superfolder GFP, AcGFP1, ZsGreen1, Monomeric Azami Green, TagGFP2, mUKG, mWasabi, Clover, mNeonGreen, Yellow Fluorescent Protein (YFP), Enhanced Yellow Fluorescent Protein (EYFP), Citrine, Venus, Super Yellow Fluorescent Protein 2 (SYFP2), TagYFP, ZsYellow1, mBanana, Orange Fluorescein Protein (OFP), Monomeric Kusabira-Orange (mKO), mKOκ, mKO2, mOrange, mOrange2, Red Fluorescent Protein (RFP), DsRed-Express, DsRed-Express2, DsRed2, AsRed2, mRaspberry, mCherry, mStrawberry, mTangerine, tdTomato, TagRFP, TagRFP-T, mApple, mRuby, mRuby2, mPlum, HcRed-Tandem, mKate2, mNeptune, HcRed1, E2-Crimson, NirFP, TagRFP657, IFP1.4, or iRFP.

In some embodiments, the fluorophore comprises xanthene, a xanthene derivative, cyanine, a cyanine derivative, squaraine, a squaraine derivative, naphthalene, a naphthalene derivative, coumarin, a coumarin derivative, oxadiazole, an oxadiazole derivative, anthracene, an anthracene derivative, a boradiazaindacine (BODIPY) family fluorophore, pyrene, a pyrene derivative, acridine, an acridine derivative, arylmethine, an arylmethine derivative, tetrapyrrole, or a tetrapyrrole derivative. For example, the fluorophore may comprise a xanthene derivative comprising fluorescein or a fluorescein derivative, rhodamine, Oregon Green, eosin, or Texas Red. Non-limiting examples of fluorescein derivatives include 5-fluorescein, 6-carboxyfluorescein, 3′6-carboxyfluorescein, 5(6)-carboxyfluorescein, 6-hexachlorofluorescein, 6-tetrachlorofluorescein, or isothiocyanate. In some embodiments, the fluorophore comprises a cyanine derivative comprising indocarbocyanine, oxacarbocyanine, thiacarbocyanine, or merocyanine. In certain embodiments, the fluorophore comprises a squaraine derivative comprising a ring-substituted squaraine. In various embodiments, the fluorophore comprises a naphthalene derivative comprising a dansyl or prodan naphthalene derivative. In a non-limiting example, the fluorophore comprises prodan or a derivative thereof. In certain embodiments, the fluorophore comprises Badan, Acrylodan, or N-(Iodoacetaminoethyl)-1-naphthylamine-5-sulfonic acid (IAEDANS). In some embodiments, the fluorophore comprises a coumarin derivative such as 3-cyano-7-hydroxycoumarin, 7-hydroxycoumarin-3-carboxylic acid, 6,8-difluoro-7-hydroxy-4-methylcoumarin (DiFMU), or 7-amino-4-methylcoumarin. In various embodiments, the fluorophore comprises an oxadiazole derivative such as pyridyloxazole, nitrobenzoxadiazole, or benzoxadiazole. In certain embodiments, the fluorophore comprises an anthracene derivative comprising an anthraquinone such as DRAQ5, DRAQ7, or CyTRAK Orange. In various embodiments, the fluorophore comprises a pyrene derivative comprising cascade blue. In non-limiting examples the fluorophore comprises an oxazine derivative such as Nile red, Nile blue, cresyl violet, or oxazine 170. In some embodiments, the fluorophore comprises an acridine derivative such as proflavin, acridine orange, or acridine yellow. In certain embodiments, the fluorophore comprises an arylmethine derivative such as auramine, crystal violet, or malachite green. In various embodiments, the fluorophore comprises a tetrapyrrole derivative comprising porphin, phthalocyanine, or bilirubin.

Aspects of the present subject matter relate to the use of fluorophores that may readily be attached to a ligand-binding protein disclosed herein, e.g., at a cysteine residue. For example, a fluorophore may comprise a sulfhydryl group prior to attachment to a ligand-binding protein that is reacted with a moiety of the ligand-binding protein to attach the fluorophore to the ligand-binding protein. In some embodiments, the fluorophore comprised a thiol group prior to attachment to the ligand-binding protein. For example, the fluorophore was thiol reactive prior to attachment to said ligand-binding protein. Non-limiting examples of fluorophores that may readily be attached to ligand-binding proteins using thiol reactions include fluorescein, pyrene, NBD, NBDE, Acrylodan (6-acryloyl-2-dimethylaminonaphthalene), Badan (6-bromo-acetyl-2-dimethylamino-naphthalene), JPW4039, JPW4042, or JPW4045.

In certain embodiments, the fluorophore comprises a derivative of a Prodan-based fluorophore such as Acrylodan or Badan. The excitation and emission properties of the Prodan-based fluorophores Acrylodan and Badan can be altered by manipulating the fluorescent ring system, while preserving the dimethylamino donor group, and the twistable carbonyl acceptor (Klymchenko 2013 Progress in Molecular Biology and Translational Science, 35-58). Replacement of the two-ring naphthalene with a three-ring anthracene (Lu 2006 J. Org. Chem., 71, 9651-9657), fluorene (Kucherak 2010 J. Phys. Chem. Lett., 1, 616-620), pyrene (Niko 2013 Chem. Eur. J., 19, 9760-9765), or styrene (Benedetti 2012 J. Am. Chem. Soc., 134, 12418-12421) cores significantly red-shift the excitation and emission properties, and in the case of the latter two, improve brightness through improvements in their excitation peak extinction coefficients. The entire content of each of the references cited above (as well as all other references referred to herein including the contents of nucleic acid and amino acid sequence accession number references) are incorporated herein by reference. Non-limiting examples of prodan analogues include 2-cyano-6-dihexylaminoanthracene and 2-propionyl-6-dihexylaminoanthracene, as well as fluorophores comprising the following structures:

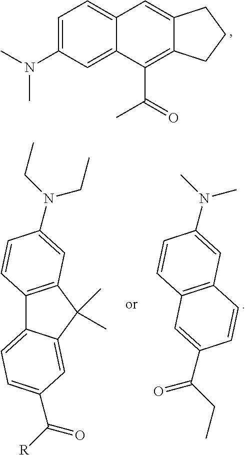

In some embodiments, the fluorophore comprises a fluorescent protein. Fluorescent proteins that emit blue, cyan, green, yellow, orange, red, far-red, or near infrared radiation when contacted with excitation radiation are known in the art and commercially available as proteins and via the expression of vectors that encode the fluorescent protein. Non-limiting examples of fluorescent proteins include Blue Fluorescent Protein (BFP), TagBFP, mTagBFP2, Azurite, Enhanced Blue Florescent Protein 2 (EBFP2), mKalama1, Sirius, Sapphire, T-Sapphire, Cyan Fluorescent Protein (CFP); Enhanced Cyan Fluorescent Protein (ECFP), Cerulean, SCFP3A, mTurquoise, mTurquoise2, monomeric Midoriishi-Cyan, TagCFP, mTFP1, AmCyan1, Green Fluorescent Protein (GFP), Enhanced Green Fluorescent Protein (EGFP), Emerald, Superfolder GFP, AcGFP1, ZsGreen1, Monomeric Azami Green, TagGFP2, mUKG, mWasabi, Clover, mNeonGreen, Yellow Fluorescent Protein (YFP), Enhanced Yellow Fluorescent Protein (EYFP), Citrine, Venus, Super Yellow Fluorescent Protein 2 (SYFP2), TagYFP, ZsYellow1, mBanana, Orange Fluorescein Protein (OFP), Monomeric Kusabira-Orange (mKO), mKOκ, mKO2, mOrange, mOrange2, Red Fluorescent Protein (RFP), DsRed-Express, DsRed-Express2, DsRed2, AsRed2, mRaspberry, mCherry, mStrawberry, mTangerine, tdTomato, TagRFP, TagRFP-T, mApple, mRuby, mRuby2, mPlum, HcRed-Tandem, mKate2, mNeptune, HcRed1, E2-Crimson, NirFP, TagRFP657, IFP1.4, or iRFP.

In some embodiments, the fluorophore comprises a quantum dot (Medintz et al. 2005) (Sapsford, Berti and Medintz 2006 Angew Chem Int Ed Engl, 45, 4562-89; Resch-Genger et al. 2008 Nat Methods, 5, 763-75). In some embodiments the emission properties of the conjugated protein are enhanced by immobilization on or near metallic nanoparticles (Zeng et al. 2014 Chem Soc Rev, 43, 3426-52; Shen et al. 2015 Nanoscale, 7, 20132-41).

In various embodiments, the peak emission wavelength and/or the emission intensity of the biosensor change when the ligand binds to the ligand-binding protein. In some embodiments, the biosensor exhibits a dichromatic signaling change when the ligand binds to the ligand-binding protein. In various embodiments, the peak emission wavelength of the biosensor shifts by at least about 5, 10, 15, 20, 30, 40, 50, or by about 5-50 nm when the biosensor binds to ligand. In certain embodiments, the emission intensity of the biosensor increases by at least about 5%, 10%, 20%, 30%, 40%, 50%, 60%, 70%, 80%, 90%, 100%, 150%, 200%, or 300% when the biosensor binds to ligand. In various embodiments, the signal produced by said reporter group persists for at least 1 nanoseconds (ns), 5 ns, 10 ns, 25 ns, 50 ns, 75 ns, 100 ns, 200 ns, 300 ns, 400 ns, 500 ns, 600 ns, 700 ns, 800 ns, 900 ns, 0.001 milliseconds (ms), 0.01 ms, 0.1 ms, 1 ms, 5 ms, 10 ms, 20 ms, 25 ms, 50 ms, 100 ms, or 500 ms when the ligand binds to the ligand-binding protein.

### Exemplary Methods of Using Biosensors Provided Herein

Aspects of the present subject matter provide a method of assaying for a ligand in a sample. The method may include contacting the sample with a biosensor disclosed herein under conditions such that the ligand-binding protein of the biosensor binds to the ligand if ligand is present in said sample. The method also comprises detecting (i) whether a signal is produced by a reporter group of the biosensor; and/or (ii) the a signal produced by a reporter group of the biosensor. In a non-limiting example, a reporter group of the biosensor is fluorescent, and the method further comprises contacting the reporter group with electromagnetic radiation having a wavelength that comprises a wavelength within the band of excitation wavelengths of the reporter group.

In various embodiments, the method further comprises (i) comparing a signal produced by a reporter group of the biosensor when the biosensor is contacted with the sample with a signal produced by a control sample containing a known quantity of ligand; and (ii) detecting the presence or absence of ligand in said sample based on this comparison. Alternatively or in addition, the method further comprises (i) comparing a signal produced by a reporter group of the biosensor when the biosensor is contacted with the sample with signals produced by a series of control samples containing known quantities of ligand; and (ii) determining the quantity of ligand in the sample based on this comparison. In some embodiments, the series of control samples comprises at least 2, 3, 4, 5, 6, 7, 8, 9, or 10 control samples, and wherein each control sample comprises a different quantity of ligand. Alternatively or in addition, the method further comprises determining the concentration of a ligand in a sample, wherein determining the concentration of the ligand in the sample comprises comparing the signal to a standard hyperbolic ligand binding curve to determine the concentration of the ligand in the test sample, wherein the standard hyperbolic ligand binding curve is prepared by measuring the signal produced by the reporter group of the biosensor when the biosensor is contacted with control samples containing known concentrations of ligand. In various embodiments, the method comprises (i) measuring a ratiometric change (ΔR) and/or an intensity change (ΔI) of a signal produced by the reporter group. In some embodiments, the method includes quantitating the level of ligand present in said sample.

In various embodiments, the ligand comprises glucose and/or galactose and said ligand-binding protein comprises a glucose-galactose binding protein.

Aspects of the present subject matter also provide a method of assaying for multiple ligands in a sample, wherein the multiple ligands comprise a first ligand and a second ligand. Such a method may include contacting the sample with (i) a first biosensor a first ligand provided herein and (ii) a second biosensor for said second ligand, under conditions such that the ligand-binding protein of said first biosensor binds to said first ligand, if said first ligand is present in said sample, and detecting (i) a signal produced by a reporter group of said first biosensor, or (ii) whether a signal is produced by a reporter group of said first biosensor. In some embodiments, the second biosensor is also a biosensor provided herein, and the second biosensor is contacted with the second ligand under conditions such that the ligand-binding protein of the second biosensor binds to the second ligand it is present in the sample. The method may further comprise detecting (i) a signal produced by a reporter group of the second biosensor, or (ii) whether a signal is produced by a reporter group of the second biosensor.

In some embodiments, the signal produced by the reporter group of said first biosensor is different than the signal produced by the reporter group of said second biosensor. In a non-limiting example, the reporter group of said first biosensor and the reporter group of said second biosensor are each fluorescent, and the peak emission wavelength of the reporter group of the first biosensor is at least about 10, 25, 50, 75, or 100 nm greater or lower than the peak emission wavelength of the reporter group of the second biosensor.

Non-limiting examples of biosensors that may be used as the second biosensor include biosensors with ligand-binding proteins comprising a GGBP (e.g., an E. coli GGBP) or a derivative or mutant thereof; (ii) an E. coli arabinose binding protein (e.g., an E. coli arabinose binding protein) or a derivative or mutant thereof; (iii) a dipeptide binding protein (e.g., an E. coli dipeptide binding protein) or a derivative or mutant thereof; (iv) a histidine binding protein (e.g., an E. coli, histidine binding protein) or a derivative or mutant thereof; (v) a ribose binding protein (e.g., an E. coli ribose binding protein) or a derivative or mutant thereof; (vi) a sulfate binding protein (e.g., an E. coli sulfate binding protein) or a derivative or mutant thereof; (vii) a maltose binding protein (e.g., an E. coli maltose binding protein) or a derivative or mutant thereof; (viii) a glutamine binding protein (e.g., an E. coli glutamine binding protein) or a derivative or mutant thereof; (ix) a glutamate/aspartate binding protein (e.g., an E. coli glutamate/aspartate binding protein) or a derivative or mutant thereof; (x) a phosphate binding protein (e.g., an E. coli phosphate binding protein) or a derivative or mutant thereof; or (xi) an iron binding protein [e.g., a Haemophilus influenza (H. influenzae) iron binding protein] or a derivative or mutant thereof. For example, the second biosensor comprises an E. coli GGBP having a Y10C, Y10A, D14A, D14Q, D14N, D14S, D14T, D14E, D14H, D14L, D14Y, D14F, N15C, F16L, F16A, F16Y, K92A, N91A, E93C, S112A, S115A, E149C, E149K, E149Q, E149S, H152A, H152F, H152Q, H152N, H152C, D154A, D154N, A155S, A155H, A155L, A155F, A155Y, A155N, A155K, A155M, A155W, A155Q, R158A, R158K, M182W, W183C, W183A, N211F, N211W, N211K, N211Q, N211S, N211H, N211M, D236A, D236N, L255C, N256A, N256D, D257C, P294C, or V296C mutation (e.g., comprising 1, 2, 3, 4, 5 or more of these mutations), wherein each amino acid position is numbered as in (SEQ ID NO: 17); (ii) an E. coli arabinose binding protein having a D257C, F23C, K301C, L253C, or L298C mutation (e.g., comprising 1, 2, 3, 4, or 5 of these mutations) (see, e.g., U.S. Patent Application Publication No. 2004/0118681, the entire contents of which are incorporated herein by reference) (see, e.g., U.S. Patent Application Publication No. 2004/0118681, the entire contents of which are incorporated herein by reference); (iii) an E. coli dipeptide binding protein having a D450C, K394C, R141C, S111C, T44C, or W315C mutation (e.g., comprising 1, 2, 3, 4, 5 or 6 of these mutations) (see, e.g., U.S. Patent Application Publication No. 2004/0118681, the entire contents of which are incorporated herein by reference); (iv) an E. coli, histidine binding protein having a E167C, K229C, V163C, Y230C, F231C, Y88C mutation (e.g., comprising 1, 2, 3, 4, 5 or 6 of these mutations) (see, e.g., U.S. Patent Application Publication No. 2004/0118681, the entire contents of which are incorporated herein by reference); (v) an E. coli ribose binding protein having a T135C, D165C, E192C, A234C, L236C, or L265C mutation (e.g., comprising 1, 2, 3, 4, 5 or 6 of these mutations) (see, e.g., U.S. Patent Application Publication No. 2004/0118681, the entire contents of which are incorporated herein by reference); (vi) an E. coli sulfate binding protein having a L65C, N70C, Q294C, R134C, W290C, or Y67C mutation (e.g., comprising 1, 2, 3, 4, 5 or 6 of these mutations) (see, e.g., U.S. Patent Application Publication No. 2004/0118681 the entire content of which is incorporated herein by reference); (vii) an E. coli maltose binding protein having a D95C, F92C, E163C, G174C, 1329C, or S233C mutation (e.g., comprising 1, 2, 3, 4, 5 or 6 of these mutations) (see, e.g., U.S. Patent Application Publication No. 2004/0118681 the entire content of which is incorporated herein by reference); (viii) an E. coli glutamine binding protein having a N160C, F221C, K219C, L162C, W220C, Y163C, or Y86C mutation (e.g., comprising 1, 2, 3, 4, 5 or more of these mutations) (see, e.g., U.S. Patent Application Publication No. 2004/0118681 the entire content of which is incorporated herein by reference); (ix) an E. coli glutamate/aspartate binding protein having a A207C, A210C, E119C, F126C, F131C, F270C, G211C, K268C, Q123C, or T129C mutation (e.g., comprising 1, 2, 3, 4, 5 or more of these mutations) (see, e.g., U.S. Patent Application Publication No. 2004/0118681 the entire content of which is incorporated herein by reference); (x) an E. coli phosphate binding protein having a A225C, N223C, N226C, S164C, or S39C mutation (e.g., comprising 1, 2, 3, 4, or 5 of these mutations) (see, e.g., U.S. Patent Application Publication No. 2004/0118681 the entire content of which is incorporated herein by reference); or (xi) a Haemophilus influenza (H. influenzae) iron binding protein having a E203C, K202C, K85C, or V287C mutation (e.g., comprising 1, 2, 3, or 4 of these mutations) (see, e.g., U.S. Patent Application Publication No. 2004/0118681 the entire content of which is incorporated herein by reference). In various embodiments, the sample is suspected of comprising glucose or galactose.

References and PDB^{a} files for bPBP structures, genes, and ligand binding

| | crystal structure | | | |
|---|---|---|---|---|
| bPBP | open form | closed form | DNA sequence | ligand affinity |
| arabinose BP | | Quiocho and | Scripture et al., | Clark et al., |
| | | Vyas, 1984 1ABE | 1987 | 1982; Miller et |
| | | | | al., 1983 |
| dipeptide BP | Nickitenko et | Dunten & | Abouhamad et | Guyer et al., |
| | al., 1995 1DPE | Mowbray, 1995 | al., 1991 | 1986; Smith et |
| | | 1DPP | | al., 1999 |
| Glu/Asp BP | | | | Barash Halpern, |
| | | | | 1975; Willis |
| | | | | Furlong, 1975 |
| Fe(III) BP | Bruns et al., | Bruns et al., 1997 | Sanders et al., | Adhikari et al., |
| | 2001 1D9V | 1MRP | 1994 | 1995 |
| glucose BP | | Vyas et al., 1988; | Scholle et al., | Anraku, 1968 |
| | | Vyas et al., 1994 | 1987 | |
| | | 1GLG | | |
| histidine BP | | Yao et al., 1994 | Joshi & Ames | Miller et al., |
| | | 1HSL | 1996 | 1983 |
| maltose BP | Sharff et al., | Spurlino et al., | Duplay et al., | Schwartz et al., |
| | 1992 1OMP | 1991; Quiocho et al., | 1984 | 1976 |
| | | 1997 1ANF | | |
| phosphate BP | Ledvina et al., | Luecke & | Magota et al., | Medveczky & |
| | 1996 1OIB | Quiocho, 1990 | 1984 | Rosenberg, 1969 |
| | | 1IXH | | |
| glutamine BP | Hsiao et al., | Sun et al., 1998 | Nohno et al., | Weiner et al., |
| | 1996 1GGG | 1WDN | 1986 | 1971 |
| ribose BP | Bjorkman & | Mowbray & Cole, | Groarke et al., | Willis & |
| | Mowbray, 1998 | 1992 2DRI | 1983 | Furlong, 1974 |
| | 1URP | | | |
| sulfate BP | | Pflugrath & | Hellinga & | Jacobson & |
| | | Quiocho, 1985; | Evans, 1985 | Quiocho, 1988 |
| | | He & Quiocho, | | |
| | | 1993 1SBP | | |

^{a}Protein Data Bank (Berman et al., 2000)
Abouhamad et al., Molec. Microbiol. 5: 1035-1047 (1991)
Adhikari et al., J. Biol. Chem. 270: 25142-25149 (1995)
Anraku, J. Biol. Chem. 243: 3116-3122 (1968)
Barash & Halpern, Biochim. Biophys. Acta 386: 168-180 (1975)
Bjorkman & Mowbray, J. Mol. Biol. 279: 651-664 (1998)
Bruns et al., Biochemistry 40: 15631-15637 (2001)
Bruns et al., Nat. Struct. Biol. 4: 919-924 (1997)
Clark et al., Biochemistry 21: 2227-2233 (1982)
Dunten & Mowbray, Protein Sci. 4: 2327-2334 (1995)
Duplay et al., J. Biol. Chem. 259: 10606-10613 (1984)
Groarke et al., J. Biol. Chem. 258: 12952-12956 (1983)
Guyer et al., J. Bacteriol. 168: 775-779 (1986)
He & Quiocho, Protein Sci. 2: 1643-1647 (1993)
Hellinga & Evans, Eur. J. Biochem. 149: 363-373 (1985)
Hsiao et al., J. Mol. Biol. 262: 225-242 (1996)
Jacobson & Quiocho, J. Mol. Biol. 204: 783-787 (1988)
Joshi & Ames, GenBank Accession Number U47027 (1996)
Ledvina et al., Proc. Natl. Acad. Sci. USA 93: 6786-6791 (1996)
Luecke & Quiocho, Nature 347: 402-406 (1990)
Magota et al., J. Bacteriol. 157: 909-917 (1984)
Medveczky & Rosenberg, Biochim. Biophys. Acta 192: 369-371 (1969)
Miller et al., J. Biol. Chem. 258: 13665-13672 (1983)
Mowbray & Cole, J. Mol. Biol. 225: 155-175 (1992)
Nickitenko et al., Biochemistry 34: 16585-16595 (1995)
Nohno et al., Molec. Gen. Genet. 205: 260-269 (1986)
Pflugrath & Quiocho, Nature 314: 257-260 (1985)
Quiocho et al., Structure 5: 997-1015 (1997)
Quiocho & Vyas, Nature 310: 381-386 (1984)
Sanders et al., Infect. Immun. 62: 4515-4525 (1994)
Scholle et al., Molec. Gen. Genet. 208; 247-253 (1987)
Scripture et al., J. Mol. Biol. 197: 37-46 (1987)
Schwartz et al., Eur. J. Biochem. 71; 167-170 (1976)
Sharff et al Biochemistry 31: 10657-10663 (1992)
Smith et al., Microbiology 145: 2891-2901 (1999)
Spurlino et al., J. Biol. Chem. 266: 5202-5219 (1991)
Sun et al., J. Mol. Biol. 278: 219-229 (1998)
Vyas et al., Biochemistry 33: 4762-4768 (1994)
Vyas et al., Science 242: 1290-1295 (1988)
Weiner et al., Arch. Biochem. Biophys. 142: 715-717 (1971)
Willis & Furlong, J. Biol. Chem. 249: 6926-6929 (1974)
Willis & Furlong, J. Biol. Chem. 250: 2574-2580 (1975)
Yao et al., Biochemistry 33: 4769-4779 (1994)

Various types of samples may be used in methods provided herein. In non-limiting examples, a sample may comprise a reaction product, a buffer, and/or a solvent. In some embodiments, the solvent is an aqueous solvent. In some embodiments, the solvent comprises a non-polar solvent, a polar aprotic solvent, and/or a polar protic solvent. For example, a sample may comprise water, liquid ammonia, liquid sulfur dioxide, sulfuryl chloride, sulfuryl chloride fluoride, phosphoryl chloride, dinitrogen tetroxide, antimony trichloride, bromine pentafluoride, hydrogen fluoride, dimethyl sulfoxide, hexane, benzene, toluene, 1,4-dioxane, chlorogorm, diethyl ether, dichloromethane, N-methylpyrrolidone, tetrahydrofuran, ethyl acetate, acetone, dimethylformamide, acetonitrile, tormic acid, n-butanol, isopropanol, nitromethane, ethanol, methanol, and/or acetic acid.

In embodiments, a sample comprises a Newtonian liquid, a shear thickening liquid, a shear thinning liquid, a thixotropic liquid, a rheopectic liquid, or a Bingham plastic. In some implementations, a sample has a dynamic viscosity of at least about 0.5, 0.6, 0.7, 0.8, 0.9, 1, 1.1, 1.2, 1.3, 1.4, 1.5, or 2 pascal-seconds (Pa·s) or less than about 2, 1.5, 1.4, 1.3, 1.2, 1.1, 1, 0.9, 0.8, 0.7, 0.6, 0.5 Pa·s; and/or a kinematic viscosity of at least about 0.5, 0.6, 0.7, 0.8, 0.9, 1, 1.1, 1.2, 1.3, 1.4, 1.5, or 2 centistokes (cSt) or less than about 2, 1.5, 1.4, 1.3, 1.2, 1.1, 1, 0.9, 0.8, 0.7, 0.6, 0.5 cSt.

In various embodiments, the sample comprises a biological sample. The sample may comprise, e.g., a clinical sample (i.e., a sample collected in a clinical or veterinary setting, e.g., by or at the request or supervision or direction of a doctor, nurse, aid worker, or medic) and/or a physiological sample (a sample collected from an organism, e.g., a mammal such as a human). In certain embodiments, the biological sample comprises or has been provided or obtained from a skin surface or a mucosal surface. In some embodiments, the biological sample comprises a biological fluid. Non-limiting examples of biological fluids include sweat, tear fluid, blood, serum, plasma, interstitial fluid, amniotic fluid, sputum, gastric lavage, skin oil, milk, fecal matter, emesis, bile, saliva, urine, mucous, semen, lymph, spinal fluid, synovial fluid, a cell lysate, venom, hemolymph, and fluid obtained from plants such as the fluid transported in xylem cells or phloem sieve tube elements of a plant (e.g. sap).

The present subject matter also provides biosensors, methods, compositions, and devices useful for measuring the level of a ligand within a liquid solution or suspension or composition comprising cultured cells or tissue or a supernatant of such a solution or suspension, e.g., a sample of conditioned media or a sample of growth media in which a population of cells was cultured. In some embodiments, the sample is within a culture (e.g., inserted into a bioreactor) or provided from a media, culture, or reaction, e.g., in a bioreactor. For example, the sample may be within or provided from a fermenter such as a culture or culture supernatant from a fermentation reaction (e.g., an ongoing fermentation). Thus, the level of a ligand can be assayed at a timepoint of interest or at a series of timepoints over the duration of cell culture, e.g. continuously, in or from a reaction or culture. Bioreactors include devices or systems that support a biologically active environment. For example, a bioreactor may comprise a vessel in which a chemical process is carried out which involves organisms or biochemically active substances derived from such organisms. Such a process can either be aerobic or anaerobic. Organisms growing in bioreactors may be, e.g., submerged or suspended in liquid medium or may be attached to the surface of a solid medium. Submerged cultures may be suspended or immobilized. Suspension bioreactors can use a wider variety of organisms, since special attachment surfaces are not needed, and can operate at much larger scale than immobilized cultures. However, in a continuously operated process the organisms will be removed from the reactor with the effluent. Immobilization is a general term describing a wide variety of cell or particle attachment or entrapment. It can be applied to basically all types of biocatalysis including enzymes, cellular organelles, and cells (e.g., animal cells, plant cells, fungal cells, and bacterial cells). Immobilization is useful for continuously operated processes, since the organisms will not be removed with the reactor effluent, but is limited in scale because the cells are only present on the surfaces of the vessel. A bioreactor may also refer to a device or system meant to grow cells or tissues in the context of cell culture. The interrogation and/or monitoring of glucose levels in such samples permits the evaluation of the status of growth of the cells or production of secreted products by the cells to inform harvest or feeding or other modification of the culture.

Aspects of the present subject matter relate to the use of methods and biosensors provided herein to detect contamination.

In some embodiments, the sample comprises an environmental sample. Depending on context, there are instances in which a biological sample may also be, or may be within, an environmental sample. In certain embodiments, an environmental sample comprises a solute obtained from a biological composition, such as bone, nail, hair, shell, or cartilage. In various embodiments, an environmental sample comprises a solute obtained from an environmental substance and/or an environmental surface. For example, the solute may be dissolved/obtained from the environmental substance and/or an environmental surface using an aqueous or nonaqueous solution. In some embodiments, an aqueous may optionally comprise a nonaqueous solvent (e.g., mixed with an aqueous solvent). Non-limiting examples of environmental substances include rock, soil, clay, sand, meteorites, asteroids, dust, plastic, metal, mineral, fossils, sediment, and wood. Non-limiting examples of environmental surfaces include the surface of a vehicle such as a civilian vehicle (e.g., a satellite, a bike, a rocket, an automobile, a truck, a motorcycle, a yacht, a bus, or a plane) or a military vehicle (e.g., a tank, an armored personnel carrier, a transport truck, a jeep, a mobile artillery unit, a mobile antiaircraft unit, a minesweeper, a Mine-Resistant Ambush Protected (MRAP) vehicle, a lightweight tactical all-terrain vehicle, a high mobility multipurpose wheeled vehicle, a mobile multiple rocket launch system, an amphibious landing vehicle, a ship, a hovercraft, a submarine, a transport plane, a fighterjet, a helicopter, a rocket, or an Unmanned Arial Vehicle), a drone, a robot, a building, furniture, or an organism other than a human. In some embodiments, the sample comprises an environmental fluid. Non-limiting examples of environmental fluids include marine water, well water, drinking well water, water at the bottom of well dug for petroleum extraction or exploration, melted ice water, pond water, aquarium water, pool water, lake water, mud, stream water, river water, brook water, waste water, treated waste water, reservoir water, rain water, and ground water. In some embodiments, waste water comprises sewage water, septic tank water, agricultural runoff, water from an area in which chemical or oil spill has or is suspected of having occurred (e.g., an oil spill into a marine environment), water from an area where a radiation leak has or is suspected of having occurred (e.g., coolant from a nuclear reactor), water within the plumbing of a building, water within or exiting a research facility, and/or water within or exiting a manufacturing facility such as a factory.

In certain embodiments, the sample comprises a food or beverage additive and/or a food or beverage composition. In some embodiments, the food or beverage composition comprises a fermented composition. In various embodiments, the sample comprises a fluid obtained from a food composition. Alternatively or in addition, the sample may comprise a solute dissolved from a food composition. In some examples, a solute is or has been dissolved from a food composition with an aqueous or nonaqueous solution. In various implementations, an aqueous solution may optionally comprise a nonaqueous solvent. In certain embodiments, a sample comprises a food composition in semisolid or liquid form. Non-limiting examples of such compositions include yogurt, soup, ice cream, a broth, a puree, a shake, a smoothie, a batter, a condiment, a sauce, and any combination thereof. In some implementations, a sample is a food engineering process (e.g., obtained from a food design, storage, transport, or production process or from equipment intended to process, transport, or store food). A food composition may comprise, e.g., a plant or a composition isolated from a plant, and/or an animal or a composition isolated from an animal. In various embodiments, a sample comprises a beverage composition. Non-limiting examples of beverage compositions include soft drinks, fountain beverages, water, coffee, tea, milk, dairy-based beverages, soy-based beverages (e.g., soy milk), almond-based beverages (e.g., almond milk), vegetable juice, fruit juice, fruit juice-flavored drinks, energy drinks, sports and fitness drinks, alcoholic products, and beverages comprising any combination thereof. Non-limiting examples of beverage compositions comprising water include purified water (e.g., filtered water, distilled water, or water purified by reverse osmosis), flavored water, mineral water, spring water, sparkling water, tonic water, and any combination thereof. In various embodiments, the sample comprises alcohol. Non-limiting examples of such samples include samples comprising or obtained/provided from beer, malt beverages, liqueur, wine, spirits, and any combination thereof.

In some embodiments, a sample comprises a nutritional or supplement composition. In certain implementations, the nutritional or supplement composition comprises an omega-3 fatty acid, a vitamin, a mineral, a protein powder, or a meal supplement.

In certain embodiments, a biosensor is implanted in a subject's body. For example, a biosensor may be implanted in a subject's blood vessel, vein, eye, natural or artificial pancreas, alimentary canal, stomach, intestine, esophagus, or skin (e.g., within the skin or under the skin). In various embodiments, the biosensor is configured within or on the surface of a contact lens. In some embodiments, the biosensor is configured to be implanted in or under the skin. In non-limiting examples, the biosensor is implanted in a subject with an optode and/or a microbead. In certain embodiments, the biosensor generates a signal transdermally.

Aspects of the present subject matter provide a method for assaying the level of glucose in a subject. The method may comprise contacting a biological sample from the subject with a biosensor for glucose under conditions such that the biosensor binds to glucose present in the biological sample. The biosensor comprises a glucose-galactose binding protein attached to a reporter group, and binding of glucose to a glucose-binding domain of the glucose-galactose binding protein causes a change in signaling by the reporter group. In various embodiments, the subject has or is suspected of having diabetes, such as Type I diabetes or Type II diabetes. In some embodiments, the biological sample comprises blood, plasma, serum, sweat, tear fluid, or urine. In certain embodiments, the biological sample is present in or on the surface of said subject. In various implementations, the biosensor is applied onto or inserted into the subject. For example, the biosensor may be tattooed into the subject or is in or on a device that is implanted into said subject. In some embodiments, the biosensor may be present in or on a contact lens that is worn by the subject. Methods for determining the level of glucose, e.g. in a subject who has or is suspected of having diabetes, may be performed without other testing related to diabetes performed as part of a battery of clinical testing.

The present subject matter includes a method for monitoring the level of a ligand, comprising periodically or continuously detecting the level of the ligand, wherein detecting the level of the ligand comprises (a) providing or obtaining a sample; (b) contacting said sample with a biosensor for said ligand according to claim 1 under conditions such that the ligand-binding protein of said biosensor binds to said ligand, and (c) detecting a signal produced by said biosensor.

Aspects of the present subject matter also provide a method for monitoring the level of a ligand (e.g., glucose and/or galactose) in a subject, comprising periodically detecting the level of the ligand in the subject. Detecting the level of the ligand in the subject may comprise (a) providing or obtaining a biological sample from said subject; (b) contacting the biological sample with a biosensor for the ligand provided herein under conditions such that the ligand-binding protein of the biosensor binds to the ligand, if the ligand is present in said biological sample, and (c) detecting (i) a signal produced by a reporter group of said biosensor, or (ii) whether a signal is produced by a reporter group of said biosensor. The level of said ligand may be detected, e.g., at least once every 1, 2, 3, 6, or 12 hours, at least once every 1, 2, 3, or 4 days, at least once every 1, 2, or three weeks, or at least once every 1, 2, 3, 4, 6, or 12 months.

The present subject matter also provides a method for monitoring the level of a ligand in a subject. The method comprises (a) administering a biosensor provided herein or a device comprising a biosensor provided herein to said subject, wherein after administration the biosensor is in contact with a bodily fluid or surface that typically comprises said ligand, and (b) detecting (i) a signal produced by a reporter group of the biosensor continuously or repeatedly at intervals less than about 30 minutes (m), 15m, 10m, 5m, 1m, 30 seconds (s), 15s, 10s, 5s, 1s, 0.1s, 0.001s, 0.0001s, or 0.00001 apart, and/or (ii) whether a signal is produced by a reporter group of the biosensor continuously or repeatedly at intervals less than about 30m, 15m, 10m, 5m, 1m, 30s, 15s, 10s, 5s, 1s, 0.1s, 0.001s, 0.0001s, or 0.00001 apart.

Non-limiting aspects of continuously monitoring glucose levels are described in Weidemaier et al. (2011) Biosensors and Bioelectronics 26, 4117-4123 and Judge et al. (2011) Diabetes Technology & Therapeutics, 13(3):309-317, the entire contents of each of which are hereby incorporated herein by reference.

Also within the invention is a composition comprising a purified thermostable, glucose-binding fluorescently-responsive sensor protein and a solid substrate, e.g., a particle, a bead such as a magnetic bead, or a planar surface such as a chip or slide, wherein the sensor protein is immobilized onto the solid substrate. An exemplary solid substrate solid substrate comprises a cyclic olefin copolymer.

The sensor protein contains a single cysteine residue, with the single cysteine residue being located in a glucose-contacting site of protein. A thermostable glucose sensor protein is one in which the activity (glucose binding) is unaffected by relatively high temperatures. For example, the glucose sensor protein comprises a mid-point thermal melt transition greater than 50° C., greater than 60° C., greater than 70° C., greater than 80ºC, greater than 90° C., or greater than 100° C. In some examples, the protein comprises the amino acid sequence of SEQ ID NO: 298, and in some examples, the single cysteine is conjugated to Badan, Acrylodan, or a derivative thereof. For example, the derivative comprises a replacement of the two-ring naphthalene of Acrylodan or Badan with a three-ring anthracene, a fluorene, or a styrene. A reporter group is covalently bound to the single cysteine. In some situations, the solid substrate comprises a plurality of sensor proteins, each of which comprises a different dissociation constant (K_{D}) for glucose, e.g., for detecting and quantifying glucose levels across many ranges of concentrations.

The invention also includes a composition comprising purified glucose sensor protein with less than 65% identity and greater than 27% identity (e.g., 44-48% sequence identity) to SEQ ID NO: 1, 16, or 17, wherein the sensor protein comprises a single cysteine residue, said residue being located in a glucose-contacting site of the protein, and a solid substrate, such that the sensor protein is immobilized onto said solid substrate. As described above, a reporter group is covalently bound to the single cysteine. In some example, the solid substrate comprises a plurality of sensor proteins, each of which comprises a different dissociation constant (K_{D}) for glucose for sensing over a wide range or ranges of glucose concentrations.

A method of detecting the presence of or the quantity of glucose in a test sample is carried out using the following steps: contacting the test sample with the biosensor or sensor protein/solid support construct to yield a complex of glucose and the ligand-binding protein or biosensor protein; contacting the complex with an excitation light; measuring an emission intensity of the reporter group from at least two wavelengths; computing a ratiometric signal from the two (or more) wavelengths; and comparing the signal to a known glucose binding curve of signals to identify the presence of or calculate the quantity of glucose in the test sample. The test sample may be obtained from a variety of sources. For example, the test sample is selected from a bodily fluid, a food, a beverage, or a bioreactor culture broth. The testing method may be carried out in vivo, e.g., using an implantable device or dermal patch, or ex vivo.

In various embodiments, the subject to be tested is a mammal, e.g., a primate (such as a human, a monkey, a chimpanzee, or a gorilla), a fish, a bird, a reptile, an amphibian, or an arthropod. In some embodiments, the subject is a fish, a cow, a pig, a camel, a llama, a horse, a race horse, a work horse, a goat, a rabbit, a sheep, a hamster, a guinea pig, a cat, a wolf, a dog (e.g., a pet dog, a work dog, a police dog, or a military dog), a rat, a mouse, a seal, a whale, a manatee, a lizard, a snake, a chicken, a goose, a swan, a duck, or a penguin.

### Exemplary Devices and Compositions Comprising Biosensors

Aspects of the present subject matter provide a device comprising one or more biosensors provided herein. Such devices may be, e.g., wearable, implantable, portable, or fixed.

In some embodiments, the device is a nanoparticle or a microparticle comprising said biosensor. Non-limiting examples of devices include devices comprising a test strip, patch, plate, bead, or chip comprising a biosensor provided herein. In certain embodiments, a device may comprise a desiccated biosensor.

The present subject matter also provides a contact lens or a skin patch comprising a biosensor provided herein. In some embodiments, the biosensor is throughout the contact lens or skin patch or within a particular region or zone of a contact lens or skin patch (e.g., in one or more shapes (e.g., a square, circle, or star), dots, lines, or zones, located at the periphery or a portion of the periphery of a contact lens or patch). In some embodiments, the skin patch comprises an adhesive that facilitates attachment of the patch to the surface of skin.

Devices provided herein may include a variety of structural compositions. For example, many polymers (including copolymers), and plastics may be used. Non-limiting examples of compositions useful in certain devices include glass, polystyrene, polypropylene, cyclic olefin copolymers, ethylene-norbornene copolymers, polyethylene, dextran, nylon, amylase, paper, a natural cellulose, a modified cellulose, a polyacrylamide, gabbros, gold, and magnetite (as well as combinations thereof). In some embodiments, the device comprises a hydrogel, a cryogel, or a soluble gel. For example, the biosensor may be incorporated into or onto the hydrogel, cryogel, or soluble gel. In various embodiments, the device comprises a matrix comprising nanopores, micropores, and/or macropores. In certain embodiments, the surface of a device comprises a polymer. In an embodiment, the surface comprises the surface of a particle or a bead having a diameter of about 0.001-1, 0.001-0.1, 0.01-0.1, 0.001-0.01, 0.1-1, 0.1-0.5, or 0.01-0.5 centimeters (cm). For example, the particle comprises a nanoparticle or a microparticle.

Non-limiting examples of polymers include cyclic olefin copolymers, ethylene-norbornene copolymers, polylactic acid, polyglycolic acid, agarose, alginate, poly(lactide-co-glycolide), gelatin, collagen, agarose, natural and synthetic polysaccharides, polyamino acids, poly(lysine), polyesters, polyhydroxybutyrates, polyanhydrides, polyphosphazines, polyvinyl alcohol, polyalkylene oxide, polyethylene oxide, polyallylamines, polyacrylates, modified styrene polymers, poly(4-aminomethylstyrene), pluronic polyols, polyoxamers, polyuronic acid, and polyvinylpyrrolidone.

In some embodiments, the device comprises a plastic polymer comprising cyclic olefin copolymer (COC), such as e.g. TOPAS® COC. Several types of cyclic olefin copolymers are available based on different types of cyclic monomers and polymerization methods. Cyclic olefin copolymers are produced by chain copolymerization of cyclic monomers such as 8,9,10-trinorborn-2-ene (norbornene) or 1,2,3,4,4a,5,8,8a-octahydro-1,4:5,8-dimethanonaphthalene (tetracyclododecene) with ethene (such as TOPAS Advanced Polymer's TOPAS, Mitsui Chemical's APEL), or by ring-opening metathesis polymerization of various cyclic monomers followed by hydrogenation (Japan Synthetic Rubber's ARTON, Zeon Chemical's Zeonex and Zeonor). See, e.g., International Union of Pure and Applied Chemistry (2005) Purr. Appl. Chem. 77(5):801-814. These later materials using a single type of monomer may be referred to as cyclic olefin polymers (COPs). A CAS Registry number for COC is 26007-43-2.

In certain embodiments, the device is attached to a surface of a device or is not attached to a surface of said device (e.g., the biosensor is present loosely within the device as a component of a solution or powder).

A biosensor may be attached to a device via a variety or means, e.g., via attachment motif. In some embodiments, the attachment motif is attached to the N-terminus or the C-terminus of the biosensor. In certain embodiments, the biosensor is linked to an attachment motif via a covalent bond. In various embodiments, the biosensor is linked to said attachment motif via a linker. A non-limiting example of a linker is a polyglycine comprising 2, 3, 4, 5, or more glycines and optionally further comprising a serine. In some embodiments, the attachment motif comprises a polypeptide. Non-limiting examples of polypeptides useful in attachment moieties include hexahistidine peptides, hexalysine peptides, zinc-finger domains (ZF-QNKs), and disulfide-containing truncated zinc fingers (βZifs). An example of a hexalysine peptide comprises amino acids in the sequence of SEQ ID NO: 296, an example of a ZF-QNK comprises amino acids in the sequence of SEQ ID NO: 294, and an example of a βZif comprises amino acids in the sequence of SEQ ID NO: 293. In some embodiments, the attachment motif comprises a polypeptide that binds to plastic or cellulose.

The hexahistidine, hexalysine, βZif and QNK-ZF fusions (SEQ ID NOS: 173 and 175-181) enable FRSs to be immobilized onto chemically functionalized surfaces. Non-limiting aspects of chemically functionalized surfaces are discussed in Biju, V. (2014) Chem Soc Rev, 43, 744-64 and McDonagh (2008) Chem Rev, 108, 400-422, the entire contents of which are incorporated herein by reference. Directed evolution methods have been used to develop peptides that bind directly to non-functionalized surfaces (Care, Bergquist and Sunna 2015 Trends Biotechnol, 33, 259-68; Baneyx 2007 Curr. Opin. Biotechnol., 18, 312-317; Gunay and Klok 2015 Bioconjug Chem, 26, 2002-15), including various plastics (Adey et al. 1995 Gene, 156, 27-31; Serizawa et al. 2005 J Am Chem Soc, 127, 13780-1; Serizawa, Sawada and Kitayama 2007a Angew Chem Int Ed Engl, 46, 723-6; Serizawa, Sawada and Matsuno 2007b Langmuir, 23, 11127-33; Serizawa, Techawanitchai and Matsuno 2007c Chembiochem, 8, 989-93; Matsuno et al. 2008 Langmuir, 24, 6399-403; Chen, Serizawa and Komiyama 2011 J Pept Sci, 17, 163-8; Kumada 2010 J. Biosci. and BioEng., 109, 583-587; Date et al. 2011 ACS Appl Mater Interfaces, 3, 351-9; Kumada 2012, Vodnik, Strukelj and Lunder 2012 J. Biotech., 160, 222-228; Kumada 2014 Biochem. et Biophys. Acta, 1844, 1960-1969; Ejima, Matsuno and Serizawa 2010 Langmuir, 26, 17278-85), inorganic materials (Hnilova 2012 Soft Matter, 8, 4327-4334; Care et al. 2015 Trends Biotechnol, 33, 259-68), nanoparticles (Avvakumova et al. 2014 Trends Biotechnol, 32, 11-20), and cellulosic paper (Guo et al. 2013 Biomacromolecules, 14, 1795-805). Such peptides, or natural material-binding domains (Oliveira et al. 2015 Biotechnol Adv, 33, 358-69), also can be fused to FRSs to direct site-specific, oriented immobilization on their target materials while preserving FRS function. For instance, plastic-binding peptides have been developed that direct immobilization on polystyrene (Adey et al. 1995 Gene, 156, 27-31; Serizawa et al. 2007c Chembiochem, 8, 989-93; Kumada 2010 Biochem. et Biophys. Acta, 1844, 1960-1969; Vodnik et al. 2012 Anal Biochem, 424, 83-6), polymethyl acrylate (Serizawa et al. 2005 J Am Chem Soc, 127, 13780-1; Serizawa et al. 2007a Angew Chem Int Ed Engl, 46, 723-6; Serizawa et al. 2007b Langmuir, 23, 11127-33; Kumada 2014 Biochem. et Biophys. Acta, 1844, 1960-1969), polycarbonate (Kumada 2012 J. Biotech., 160, 222-228), polylactide (Matsuno et al. 2008 Langmuir, 24, 6399-403), and polyphenylene vinylene (Ejima et al. 2010 Langmuir, 26, 17278-85). Cellulose-binding peptides (Guo et al. 2013 Biomacromolecules, 14, 1795-805) and natural domains (Oliveira et al. 2015 Biotechnol Adv, 33, 358-69; Shoseyov, Shani and Levy 2006 Microbiol Mol Biol Rev, 70, 283-95) can be used to immobilize fusion proteins on paper. Inorganic material include noble metals (Hnilova 2012 Soft Matter, 8, 4327-4334), semi-conductors (Care et al. 2015 Trends Biotechnol, 33, 259-68), and fluorescent quantum dots (Medintz et al. 2005 Nat Mater, 4, 435-46; Lee et al. 2002 Science, 296, 892-5). The entire contents of each of the references above (and all other references herein) is incorporated herein by reference.

In some embodiments, the attachment motif is attached to a device surface and/or within a matrix of the device. In some embodiments, a biosensor is attached to an attachment motif via a covalent bond and the attachment motif is attached to a device via a covalent bond. Non-limiting examples of covalent bonds include disulfide bonds, ester bonds, thioester bonds, amide bonds, and bonds that have been formed by click reactions. Non-limiting examples of a click reaction include a reaction between an azide and an alkyne; an azide and an alkyne in the presence of Cu(I); an azide and a strained cyclooctyne; an azide and a dibenzylcyclooctyne, a difluorooctyne, or a biarylazacyclooctynone; a diaryl-strained-cyclooctyne and a 1,3-nitrone; an azide, a tetrazine, or a tetrazole and a strained alkene; an azide, a tetrazine, or a tretrazole and a oxanorbornadiene, a cyclooctene, or a trans-cycloalkene; a tetrazole and an alkene; or a tetrazole with an amino or styryl group that is activated by ultraviolet light and an alkene.

Alternatively or in addition, a surface of a device may be modified to contain a moiety (e.g. a reactive group) what facilitates the attachment of a biosensor and/or binds to the biosensor. In some embodiments, the biosensor is attached to a surface via a biotin-avidin interaction.

In various implementations, the device comprises a first region for receiving a sample and second a region that comprises the biosensor, wherein said first region is separated from said second region by a filter. In some examples, the filter is impermeable to compounds greater than about 1, 2, 3, 4, 5, 10, 50, 200, or 250 kiloDalton (kDa) in size. The sample may comprise, e.g., a tube, such as a tube that is configured for centrifugation. When sample is placed into the first region and the device is centrifuged, then a portion of the sample comprising a ligand flows through the filter into the second region where the biosensor is contacted.

Non-limiting examples of devices provided herein include endoscopy probes and colonoscopy probes.

In some embodiments, the device comprises an optode. In non-limiting examples, the optode comprises an optical fiber and a single biosensor or composite biosensor. In certain embodiments, the single biosensor or composite biosensor is immobilized on the surface or at an end of the optical fiber. In some embodiments, the optode is configured for implantation into a subject. Alternatively or in addition, the optode is configured for insertion into a sample.

The devices provided herein may optionally comprise a biosensor panel, a composite sensor, a sensor array, and/or a composition comprising a plurality of biosensors. In various embodiments, a device comprises multiple glucose and/or galactose biosensors that detect a range of different glucose and/or galactose concentrations in a single sample and/or assay run (i.e., each biosensor has a different affinity for glucose and/or galactose). Devices may provide spatial localization of multiple biosensors to provide the necessary addressability of different elements in a multi-sensor array comprising sensors that differ in their engineered affinities for coverage of a wide range of glucose concentrations, or sensors that each detects distinct analytes.

Aspects of the present subject matter provide a biosensor panel comprising a plurality of biosensors, wherein said plurality of biosensors comprises at least one biosensor disclosed herein. In some embodiments, the plurality comprises at least about 2, 3, 4, 5, 10, 20, 30, 40, 50, 60, 70, 80, 90, or 100 biosensors.

The present subject matter also provides a composite sensor. The composite sensor may comprise a sensor element, wherein the sensor element comprises 2 or more biosensors, wherein at least 1 of said 2 or more biosensors is a biosensor disclosed herein. In some embodiments, the biosensors are not spatially separated in the sensor element, e.g., the biosensors are mixed within a solution or on a surface of the sensor element. In various embodiments, the composite sensor comprises a plurality of sensor elements, wherein each sensor element of the plurality of sensor elements comprises 2 or more biosensors, wherein at least 1 of the 2 or more biosensors is a biosensor provided herein. In some embodiments, the plurality of sensor elements comprises at least about 2, 3, 4, 5, 10, 20, 30, 40, 50, 60, 70, 80, 90, or 100 sensor elements.

Also included herein is a sensor array comprising a plurality of biosensors of the present subject matter. The sensor array may include, e.g., multichannel array or a multiplexed array. In some embodiments, the biosensors of the plurality of biosensors are spatially separated from each other. In certain embodiments, the biosensors are arranged linearly or in a grid on a surface of said array.

The present subject matter provides a composition comprising a plurality of biosensors including at least one biosensor disclosed herein. Also provided is a non-human mammal comprising a biosensor or device disclosed herein.

### Exemplary Polypeptides and Polynucleotides

The present subject matter provides polynucleotides encoding any one of the polypeptides disclosed herein. The polypeptides are also provided. In various embodiments, the polynucleotides are codon-optimized for expression in a desired host cell, such as bacterial cells (e.g., E. coli), yeast, insect cells, plant cells, algal cells, or mammalian cells. The polypeptides provided herein include polypeptides comprising the amino acid sequence of any one of SEQ ID NOS: 1-103, 157-292, 197, or 198. The polynucleotides provided herein include polynucleotides encoding a polypeptide comprising the amino acid sequence of any one of SEQ ID NOS: 1-103, 157-292, 197, or 198.

The polypeptides and biosensors provided herein may be in a variety of forms, e.g., purified in solution, dried (e.g. lyophilized) such as in the form of a powder, and in the form of a crystal (e.g., a crystal suitable for x-ray crystallography). Thus, aspects of the present subject matter provide crystal structures and crystalized forms of the ligand-binding proteins and biosensors disclosed herein. Such crystal structures and crystalized proteins are useful for designing and optimizing biosensors using principles and methods discussed herein.

Also provided are expression vectors comprising a polynucleotide of the present subject matter and/or encoding a polypeptide disclosed herein. Non-limiting examples of expression vectors include viral vectors and plasmid vectors. In some embodiments, an expression vector comprises nucleotides in the sequence set forth as SEQ ID NO: 105-156. In various embodiments, a polynucleotide encoding a ligand-binding protein and/or biosensor is operably linked to a promoter. The promoter may be expressed, e.g., in a prokaryotic and/or a eukaryotic cell.

The subject matter further includes an isolated cell comprising an expression vector provided herein. The isolated cell may be, e.g., a bacterial cell, a yeast cell, an algal cell, a plant cell, an insect cell, or a mammalian cell. Also included is a non-human multicellular organism such as a plant or an animal (e.g., an insect, a mammal, a worm, a fish, a bird, or a reptile) comprising an expression vector disclosed herein.

### Exemplary Methods for Designing Biosensors

Aspects of the present subject matter provide method of identifying a candidate ligand-binding protein for use in a biosensor, comprising: (a) selecting a first protein having a known amino acid sequence (seed sequence), wherein the first protein is a glucose-galactose binding protein; (b) identifying a second protein having an amino acid sequence (hit sequence) with at least 15% sequence identity to the seed sequence; (c) aligning the seed amino acid sequence and the hit sequence, and comparing the hit sequence with the seed sequence at positions of the seed sequence that correspond to at least 5 primary complementary surface (PCS) amino acids, wherein each of the at least 5 PCS amino acids has a hydrogen bond interaction or a van der Waals interaction with glucose when glucose is bound to the first protein; and (d) identifying the second protein to be a candidate ligand-binding protein if the hit sequence comprises at least 5 amino acids that are consistent with the PCS.

The present subject matter also includes a method for constructing a candidate biosensor, comprising: (a) providing a candidate ligand-binding protein; (b) generating a structure of the second protein; (c) identifying at least one putative allosteric, endosteric, or peristeric site of the second protein based on the structure; (d) mutating the second protein to substitute an amino acid at said at least one putative allosteric, endosteric, or peristeric site of the second protein with a cysteine; and (e) conjugating a fluorescent compound to the cysteine. In some embodiments, the structure comprises a homology model of the second protein generated using a structure of the first protein. In some embodiments, the structure comprises a structure experimentally determined by nuclear magnetic resonance spectroscopy or X-ray crystallography.

Aspects of the present subject matter further provide a method for constructing a biosensor comprising a desired dissociation constant (KD) for glucose, comprising: (a) providing an initial biosensor that does not comprise the desired KD for glucose, wherein said initial biosensor is a biosensor provided herein; (b) mutating the initial biosensor to (i) alter a direct interaction in the PCS between the initial biosensor and bound glucose; (ii) manipulate the equilibrium between open and closed states of the initial biosensor; (iii) alter an interaction between the ligand-binding protein and the reporter group of the initial biosensor; or (iv) alter an indirect interaction that alters the geometry of the binding site of the biosensor, to produce a modified biosensor; and (c) selecting the modified biosensor if the modified biosensor comprises the desired KD for glucose. In some embodiments, the reporter comprises Acrylodan, Badan, or a derivative thereof, and mutating the initial biosensor in (b) comprises altering an interaction between the ligand-binding protein and a carbonyl group of the Acrylodan, Badan, or derivative thereof. In some embodiments, the reporter group comprises Acrylodan, Badan, or a derivative thereof, and mutating the initial biosensor in (b) comprises altering an interaction between the ligand-binding protein and a naphthalene ring of the Acrylodan, Badan, or derivative thereof. In some embodiments, mutating the initial biosensor comprises introducing a substitution mutation into said initial biosensor. In some embodiments, the method further comprises immobilizing said affinity-tuned biosensor on a substrate.

In some embodiments, the second protein comprises (i) amino acids in the sequence of SEQ ID NO: 1-15 or 17-31; (ii) a stretch of amino acids in a sequence that is least about 95, 96, 97, 98, or 99% identical to the sequence of SEQ ID NO: 1-31; (iii) a stretch of at least about 50, 100, 150, 200, 250, 300, or 350 amino acids in a sequence that is at least about 95, 96, 97, 98, or 99% identical to a sequence within SEQ ID NO: 1-31; or (iv) a stretch of at least about 50, 100, 150, 200, 250, 300, or 350 amino acids in a sequence that is identical to a sequence within SEQ ID NO: 1-31. In various embodiments, attaching the reporter group to the putative allosteric, endosteric, or peristeric site of the first protein comprises substituting a cysteine at the site with a cysteine. For example, the reporter group is conjugated to the cysteine. Preferably, attaching a reporter group to the corresponding amino acid of the second protein produces a functional biosensor.

The selected first protein (e.g., the amino acid sequence thereof) may be novel or known. However, in many instances, the function of the first protein will not be known. In a non-limiting example, identifying a protein not previously known to have glucose binding activity may comprise a structurally assisted functional evaluation (SAFE) homolog search method comprising the following steps:
(1) Collecting a sequence homology set using a BLAST sequence alignment tool starting with glucose-galactose binding protein (GGBP) sequence disclosed herein as a seed. Permissive settings are used, such that pairwise hits are required to have a minimum of only, e.g., 20%, 25%, 30%, 35% or 40% sequence identity with the seed sequence. The lengths of the hit and seed are mutually constrained such that the alignment covers at least, e.g., 60%, 65%, 70%, 85%, or 90% within each partner.
(2) Structure-based encoding of biological function: A primary complementary surface (PCS) comprising the protein residues that form hydrogen bonds and van der Waals contacts with a bound glucose is defined using computer-assisted, visual inspection of the three-dimensional structure of the GGBP-glucose complex. This definition specifies residue positions and their permitted amino acid identity. Multiple amino acid identities are permitted at each position to encode functionally equivalent residues. This definition establishes a search filter for the accurate prediction of glucose-binding proteins within the universe of sequence homologs collected in (1). For example, a candidate's residue corresponding to position 14 of ecGGBP may be D or N, a candidate's residue corresponding to position 16 of ecGGBP may be F, Y, or W, a candidate's residue corresponding to position 91 of ecGGBP may be N or D, a candidate's residue corresponding to position 152 of ecGGBP may be H, N, or Q, a candidate's residue corresponding to position 154 of ecGGBP may be D or N, a candidate's residue corresponding to position 158 of ecGGBP may be R, a candidate's residue corresponding to position 183 of ecGGBP may be W, F, or Y, a candidate's residue corresponding to position 211 of ecGGBP may be N or D, a candidate's residue corresponding to position 236 of ecGGBP may be D or N, and a candidate's residue corresponding to position 256 of ecGGBP may be N or D.
(3) Accurate sequence alignment: Tools such as ClustalW are used to construct an accurate alignment of all the sequence homologs. The GGBP seed sequence is included in the alignment. This multiple sequence alignment establishes the equivalent positions of the seed GGBP (primary complementary surface) PCS in each sequence homolog.
(4) Function evaluation: The glucose-binding properties of each of the aligned sequence homologs is determined by measuring their compliance with the PCS sequence filter. A “Hamming distance”, H, is assigned for each homolog, which specifies the degree of sequence identity of all the residues at the aligned PCS positions. A value of H=0 indicates that the identities of all the residues at the aligned PCS positions match the amino acid(s) allowed in the PCS search filter; H>0, indicates that one or more aligned positions have disallowed residues. Sequences for which H=0 are predicted to encode glucose-binding proteins.
(5) Selection of representative SAFE homologs: The sequence homologs are ordered by (a) identity with the seed PCS, as measured by the Hamming distance, (b) fractional overall sequence identity with the seed sequence. A subset for sequences with H=0, sampling the fractional overall sequence identity is selected for experimental verification.

In a non-limiting example, identifying a protein not previously known to have glucose binding activity may comprises the following steps:
(1) performing a computational search of sequence databases to define a broad group of simple sequence or structural homologs of any known, glucose-galactose binding protein;
(2) using the list from step (1), deriving a search profile containing common sequence and/or structural motifs shared by the members of the list [e.g. by using computer programs such as MEME (Multiple Em for Motif Elicitation available at meme.sdsc.edu/meme/cgi-bin/meme.cgi) or BLAST];
(3) searching sequence/structural databases, using a derived search profile based on the common sequence or structural motif from step (2) as query (e.g., using computer programs such as BLAST, or MAST (Motif Alignment Search Tool available at meme.sdsc.edu/meme/cgi-bin/mast.cgi), and identifying a candidate sequence, wherein a sequence homology and/or structural similarity to a reference glucose-galactose binding protein is a predetermined percentage threshold;
(4) compiling a list of candidate sequences to generate a list of candidate glucose-galactose binding proteins;
(5) expressing the candidate glucose-binding proteins in a host organism; and
(6) testing for glucose and/or galactose binding activity, wherein detection of glucose and/or galactose binding in said organism (or the media thereof) indicates that the candidate sequence comprises a novel glucose and/or galactose binding protein.

In non-limiting examples, the MEME suite of sequence analysis tools (meme.sdsc.edu/meme/cgi-bin/meme.cgi) can also be used as an alternative to BLAST. Sequence motifs are discovered using the program “MEME”. These motifs can then be used to search sequence databases using the program “MAST.” The BLAST search algorithm is well-known.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

FIGS. 1A-C are a cartoon and graphs illustrating emission spectra changes upon ligand binding to FRSs. (A) FRSs can be constructed by site-specifically attaching a fluorophore to a protein that undergoes a conformational change upon binding ligand (triangle), such that the shape and intensities of the fluorescent conjugate emission spectra changes. (B) In the absence of ligand, the emitted fluorescence color is predominantly blue, whereas the ligand complex fluoresces green. Arrows indicate the direction of change upon ligand addition. (C) Ligand binding is determined by measuring the ratio of blue to green emission intensities.

FIGS. 2A-C are structural illustrations and a table showing exemplary features of the E. coli glucose-galactose binding protein (ecGGBP). (A) The glucose and calcium complex [PDB identifier 2gbp (Vyas et al. 1988)], glucose is sandwiched by van der Waals contacts with the rings of Phe16 and Trp183. (B) Residues that form hydrogen bonds with the bound glucose. The primary complementary surface (PCS) comprises the aromatic sandwich and the hydrogen-bonding residues. (C) The PCS sequence filter used to identify the subset of glucose-binding proteins within a family of sequence ecGGBP homologs. Note redundancies in the allowed residues at each position (the first amino acid listed corresponds to the wild-type ecGGBP sequence).

FIG. 3 is an alignment of the homologs predicted to be glucose-binding proteins (alignment generated by ClustalW; ordered by fractional sequence identity to the ecGGBP seed sequence). Sequences taken from Table 1 (name, line number in Table 1, accession code, species, fractional identity to ecGGBP):
ecGGBP, 2, NC_013654|YP_003350022.1, Escherichia coli, 100%;
ttGGBP, 96, NC_014410|YP_003852930.1, Thermoanearobacterium thermosaccharolyticum, 48%;
cobGGBP, 94, NC_014392|YP_003839461.1, Caldicellulosiruptor obsidiansis, 48%;
chyGGBP, 101, NC_014652|YP_003991244.1, Caldicellulosiruptor hydrothermalis, 47%;
pspGGBP, 112, NC_013406|YP_003243743.1, Paenibacillus sp., 44%;
ereGGBP, 123, NC_012781|YP_002936409.1, Eubacterium rectale, 37%;
cauGGBP, 127, NC_022592|CAETHG_2982, Clostridium autoethanogenum, 36%;
erhGGBP, 124, N_015601|YP_004561181.1, Erysipelthrix rhusiopathiae, 36%;
rinGGBP_B, 126, NC_021012|YP_00777811241, Roseburia intestinalis, 36%;
cljGGBP, 128, NC_014328|CLJU_c08950, Clostridium ljunghaldii, 35%;
fprGGBP, 129, NC_021020|YP_007799070.1, Faecalibacterium prausnitzii, 34%;
rinGGBP_A, 132, NC_021012|YP_007778116.1, Roseburia intestinalis, 33%;
bprGGBP, 137, NC_014387|YP_003830205.1, Butyvibrio proteoclasticus, 30%;
csaGGBP, 138, NC_014376|YP_003822565.1, Clostridium saccharolyticum, 29%.
   Numbering according to ecGGBP. Light grey: leader peptides; dark grey, primary complementary surface (PCS) residues; -, position of insertions. Positions of the α helices (αₓ), β sheets (βₓ), inter-domain hinge segments (hₓ), and calcium binding site EF hand (EF) observed in the ecGGBP structure are indicated.

FIGS. 4A and 4B are graphs relating to the analysis of glucose binding by ligand-mediated increases in protein thermostability. Protein (un)folding is monitored by binding of SYPRO to the unfolded state as a function of temperature. Fluorescence intensity increases upon binding of SYPRO by the unfolded state. (A) Temperature dependence of fluorescence emission intensity of csaGGBP (see Tables 1 and 2) in the presence of varying amounts of glucose. (B) The mid-point denaturation temperature, Tₘ, increases upon addition of glucose, indicative of glucose binding to the folded state of csaGGBP, as predicted

FIG. 5 is a series of graphs showing temperature- and glucose-dependent ratiometric fluorescent landscapes of the Acrylodan conjugates of GGBP homologs. Rows correspond to the E. coli (ecGGBP), T. thermosaccharolyticum (ttGGBP), C. obsidiansis (cobGGBP), and C. hydrothermalis (chyGGBP) glucose-galactose binding proteins site-specifically labeled with Acrylodan at the endosteric cysteine mutation replacing the tryptophan that contacts the glucose pyranose ring (W183 in ecGGPB). Column I: Three-dimensional landscapes representing the ratio of fluorescence emission intensities (Z axis) at 488 nm and 510 nm as a function of temperature and glucose concentration. Indicated are the main equilibrium states: N, native apo-protein; D, denatured protein; S, saturated glucose complex. Subscripts indicate temperature-dependent conformational sub-states (see main text). Dotted lines indicate approximate mid-points (i.e. ΔG=0) for the equilibria of the underlying reactions: black lines (----), binding; red (-⋅-⋅-), thermal denaturation; purple (⋅⋅⋅⋅), thermal conformational changes. Column II: Examples of isothermal glucose-binding curves, determined at 310 K (37° C.). For each protein, two panels are shown: top, fit to the ratiometric signal (red line, fit to the ratiometric data only; blue line, joint fit to ratiometric and monochromatic emission intensities); bottom, fit to monochromatic emission intensities (red, 488 nm; blue, 510 nm).

FIG. 6A is a structure showing exemplary positions for introducing cysteine mutations to which fluorophores can be covalently coupled for reagentless biosensor construction are indicated: positions 157, 159, and 300, endosteric; positions 11, 16, 42, 67, 92, 111, 148, 181, and 183, peristeric; positions 17, 91, 151, and 182, allosteric. FIG. 6B shows a structure of Acrylodan. FIG. 6C shows a structure of Badan.

FIG. 7A-F is a series of graphs for glucose-dependent emission spectra of Acrylodan and Badan conjugates of Y11C, F17C and W182C mutants of ttGGBP. Corrected spectra (apo-protein, red; saturated glucose, purple; intermediate glucose concentrations, black): left column, Acrylodan; right column, Badan. Insets, fit of the ratiometric signal (equation 1 and 2; 20 nm integration bandwidth): blue circles, experimentally observed ratios; black line, calculated fit (baselines; apo-protein, constant; saturated glucose complex, linear). (A) Y11C. Acrylodan (isochromic; λ₁, 458 nm; λ₂, 511 nm; ^{app}K_{d}, 0.14 mM); (B) Y11C⋅Badan (hypsochromic; λ₁, 492 nm; λ₂, 528 nm; ^{app}K_{d}, 0.28 mM); (C) F17C⋅Acrylodan (hypsochromic; λ₁, 482 nm; λ₂, 545 nm; ^{app}K_{d}, 0.08 mM); (D) F17C⋅Badan (hypsochromic; λ₁, 470 nm; λ₂, 542 nm; ^{app}K_{d}, 0.18 mM); (E) F182C⋅Acrylodan (bathochromic; λ₁, 475 nm; λ₂, 545 nm; ^{app}K_{d}, 2.2 mM); (F) F182C. Badan (isochromic; λ₁, 474 nm; λ₂, 518 nm; ^{app}K_{d}, 26 mM).

FIGS. 8A-P are illustrations of fluorophore structures. Naphthalene family: (A) Acrylodan; (B) Badan; (C) IAEDANS. Xanthene family: (D) Fluorescein (5-IAF and 6-IAF); (E) Oregon Green; (F) Alexa 432; (G) Alexa 532; (H) Alexa 546; (I) Texas Red. Coumarin family: (J) Pacific Blue; (K) CPM. Benzoxadiazole family: (L) IANBD. Boradiazaindacine (BODIPY) family: (M) BODIPY 499/508; (N) BODIPY 507/545. Cyanine family: (O) Cy5. Miscellaneous: (P) PyMPO.

FIGS. 9A-H are illustrations of structures of ttGGBP conjugates determined by X-ray crystallography. (A) Overview of the ttGGBP F17C⋅Badan conjugate, viewed from the back of the protein looking through the three hinge strands (h₁-h₃). W182 (magenta) forms extensive van der Waals contacts (translucent surface) with the bound glucose (cyan). The F17C mutation removes the wild-type contacts (translucent surface) between the benzene ring and glucose. The Badan conjugate (blue) points out of the glucose-binding site into the solvent. (B) Overview of the ttGGBP182C2.0⋅Acrylodan conjugate (note that the view point is different from (A). The conjugated Acrylodan also points out into solution. (C) Cutaway illustrating the channels surrounding the bound glucose within which Badan, Acrylodan, and the galactoside R-group (Sooriyaarachchi et al. 2009) (dark gray) are placed. The view is down the long axis of ttGGBP, looking at the glucose-binding surface of the N-terminal domain. The C-terminal domain (with Ca²⁺ site) has been cut away (bisecting the hinge). (D) The electronic densities of the Badan fluorophore and bound sugar are well defined (gray lines, 2Fₒ-F_{c} electron density at 1 σ contour levels). (E) Close-up side-view of Badan illustrates that the fluorophore carbonyl is twisted out the of the naphthalene ring plane. (F) End view, looking down the linker towards the exterior of the binding site shows that the carbonyl torsion is ˜28° (gray lines, 2Fₒ-F_{c} electron density at 1 σ contour levels; arrow, cysteine thiol). (G) The same view of the ecGGBP W183C⋅Acrylodan shows that in this fluorophore the carbonyl is co-planar with the naphthalene ring (blue lines, 2Fₒ-F_{c} electron density at 1 σ contour levels; arrow, cysteine thiol). (H) Close-up view of the fit between the fluorophore carbonyl knob into the hole (translucent surface) formed by van der Waals contacts with D238 and N258.

FIGS. 10A and B are structures relating to structure-guided design of affinity mutants. (A) The ttGGBP182C2.0 Acrylodan conjugate structure reveals the presence of a cavity filled with water and the cryoprotectant ethylene glycol, replacing the indole ring of the mutated tryptophan. (B) Inter-domain hydrogen bonds that have been mutated.

FIGS. 11A-C are graphs relating to the determination of ^{true}K_{d} and ^{app}K_{d} values for glucose in ttGGBP182C.2.0 (see Table 5). (A) Glucose dependence of the fluorescence emission intensities for the ttGGBP182C.2.0⋅Acrylodan mutant conjugate (see Table 4). Arrows indicate direction of change with increased glucose concentrations. (B) Determination of ^{app}K_{d} using ratiometry the emission intensities (R) at 475 nm and 545 nm (20 nm bandwidth). (C) Determination of ^{true}K_{d} by monochromatic fits the two ratiometric intensities (I(λ): green, 475 nm; red, 545 nm). Values for ^{app}K_{d} (4.5 mM) and ^{true}K_{d} (6.0 mM) were simultaneously fit to the three experimental binding isotherms using equations 1 and 5 using constant baselines for both the apo- and saturated protein, with the two monochromatic isotherms sharing the same ^{true}K_{d} value.

FIGS. 12A and B are graphs showing that a sensor arrays that combine several ttGGBP mutants (Table 6) could span the full pathophysiological range at better than 90% of maximal possible precision. (A) Mutants in the ttGGBP182C Acrylodan background. The responses are shown as relative precision, calculated from the fits to the experimental data according to equation 8, from left to right: ttGBP182C, hypoglycemia; ttGGBP182C.2.0, euglycemia; ttGGBP182C.2.3, mild hyperglycemia; ttGGBP182C.8, severe hyperglycemia; ttGGBP182C.9, hyperosmolar hyperglycemic state. (B) Mutants in the ttGGBP17C Badan background. Ratiometric signals are shown from left to right: ttGGBP 17C⋅Acrylodan (^{app}K_{d}=0.06 mM), ttGGBP 17C.1⋅Badan (^{app}K_{d}=1.2 mM), ttGGBP 17C.3⋅Badan (^{app}K_{d}=3.9 mM), ttGGBP 17C.5⋅Badan (^{app}K_{d}=19.3 mM). Black circles: experimental data points

FIG. 13 is a set of cartoons showing fusion constructs for sensor immobilization. Light gray, ttGGBP182C.2.0; diagonal striped, hexa-lysine immobilization tag; dark gray, hexa-histidine affinity purification tag; horizontal striped, ZF-QNK zinc finger domain; vertical striped, truncated zinc finger βZif domain; wavy line, Gly-Gly-Ser linker (two segments, indicate Gly-Gly-Ser-Gly-Gly-Ser). Left column, names of constructs.

FIGS. 14A-F are graphs showing that the immobilization of ttGGBP182C2.0⋅Acrylodan does not affect its thermostability. Thermostabilities were determined by measuring the ratio fluorescence emission intensities through 488 nm and 510 nm filters as a function of temperature in a Roche LightCycler. Panels A-C: ttGBP182C.2.0-Imm0; Panels D-F: ttGBP182C.2.0-Imm1 (see FIG. 13). (A) Solution (Tₘ=347.6K). (B) Immobilized on Nickel-nitriloacetic acid (Ni-NTA) beads (Tₘ=346.7K). (C) Reconstituted, desiccated Ni-NTA beads (Tₘ=347.6K). (D) Fluorescence response of ttGGBP5.2 Imm1 immobilized on NHS-functionalized agarose beads. Data was collected on a Roche LightCycler real-time PCS instrument, recording emission intensities at 488 nm and 510 nm as a function of temperature in a 384-well microtiter plate. Wells contain beads with labelled, immobilized protein in hydrogel at different glucose concentrations. Temperature melts of twelve different wells. Apo protein thermostability as reported by the denaturation mid-point temperature is 349 K, similar to the protein free in solution. (E) The three-dimensional landscape showing ratiometric signal as a function of glucose concentration and temperature is essentially indistinguishable from the protein free in solution. (F) Representative Langmuir ligand-binding curve extracted from the three-dimensional data. The K_{d} (glucose) value at 25° C. is 4.8 mM, which is similar to the protein free in solution.

FIG. 15 is a graph of a glucose titration curve determined for magnetic Ni-NTA beads coated with immobilized ttGGBP182C2.0⋅Acrylodan. Data were highly precise, being reproducible <0.2% coefficient of variance.

FIGS. 16A-F are graphs showing the singular value decomposition of the glucose-dependent corrected spectra of Acrylodan and Badan conjugates of Y11C, F17C and W182C mutants of ttGGBP. (A) Y11C⋅Acrylodan; (B) Y11C⋅Badan; (C) F17C⋅Acrylodan; (D) F17C⋅Badan; (E) F182C⋅Acrylodan; (F) F182C⋅Badan. Frequency transformations of the spectra (equation 11) were decomposed into principal components (equation 12). The contribution of the first component, C1 (black), is largely invariant with glucose concentration, whereas the second component, C2 (gray), encode accounts for the glucose-dependent changes in the spectra (component contributions are given in Table 1). Inserts, glucose dependence of the fractional contribution of each component (equation 13).

FIGS. 17A-F are graphs showing glucose dependence of electronic transitions in the fluorescence emission intensity spectra of Acrylodan and Badan conjugates of Y11C, F17C and W182C mutants of ttGGBP. (A) Y11C⋅Acrylodan; (B) Y11C⋅Badan; (C) F17C⋅Acrylodan; (D) F17C⋅Badan; (E) F182C⋅Acrylodan; (F) F182C⋅Badan. The emission intensities can be accounted for to a first approximation by two excited state electronic transitions (FIG. 16): a low-energy, green, S₁ (Acrylodan: 521±9 nm; Badan: 530±14 nm) and a high-energy, blue, S₂ (Acrylodan: 477±14 nm; Badan: 477±16 nm) transition. The S1 transition is present in all spectra; S₂ is present in all the glucose-responsive and 36% of the non-responsive conjugates (Table 7 and 8). In conjugates that exhibit glucose-responsive changes in fluorescence emission intensities, glucose binding shifts the population of these excited states. At each titration point, the experimentally observed emission intensities were modeled with Gaussians fits (equation 14) for S₁ and S₂ electronic transition (model parameter values in Table 3). Experimental emission spectra and Gaussian fits are shown only for the apo-protein, and saturated glucose complex. Emission spectra: dashed black line, apo-protein; solid black line, glucose complex. Gaussians (S₁, green lines; S₂, blue lines): dashed lines, apo-protein; solid lines, glucose complex. Thin black lines: residuals (equation 16) at each titration point. Inserts show the population fractions (equation 15) of the S₁ and S₂ transitions extracted from the spectra at each titration point (black circles) fit to Langmuir binding isotherms (solid lines) with ^{app}K_{d} values constrained to be the same for both populations. As a first approximation, the wavelengths of S₁ or S₂ transitions are the same in apo-protein and the saturated glucose complex. The residuals indicate that this approximation does not hold for all conjugates, and a more extensive treatment is required in which the S₁ and S₂ are split into multiple transitions (see discussion below). Wavelength shifts occur if there is a significant redistribution of the two excited state populations in the apo-protein and the saturated ligand complexes. In the bathochromic ratiometric 182C⋅Acrylodan conjugate, the S₁ state dominates in the glucose complex (panel E); in the hypsochromic conjugates 17C⋅Badan (panel D) and 17C⋅Acrylodan (panel C), apo-proteins comprise a mixture of the two states, whereas their glucose complexes contain almost exclusively the S₂ state. In the hypsochromic 11C⋅Badan sensor (panel B), the system shifts from a predominantly S₁ in the absence of glucose to almost equally populated S₁ and S₂ states in the glucose complex. Shifts in the fraction of S₁ and S₂ populations also occur in conjugates that did not exhibit overt shifts in the wavelengths of their emission intensity maxima. For instance, neither 11C⋅Acrylodan nor 182C⋅Badan exhibited significant shifts in emission maxima upon binding glucose. Nevertheless, SVD analysis revealed similar changes in the variable C2 component as observed for the overtly ratiometric sensors, but with a smaller contribution (FIGS. 16A and F). Gaussian analysis revealed small shifts in the S₁ and S₂ populations in response to glucose (panel A and F). This pattern also was observed in other non-ratiometrically responsive conjugates (Table 7 and 8). Wavelength shifts were observed if the C₂ component constitutes >5% of the total contribution.

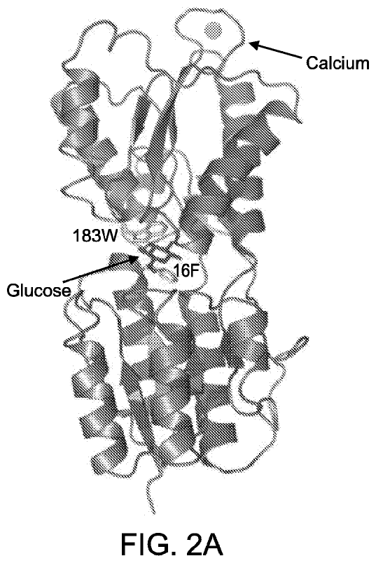

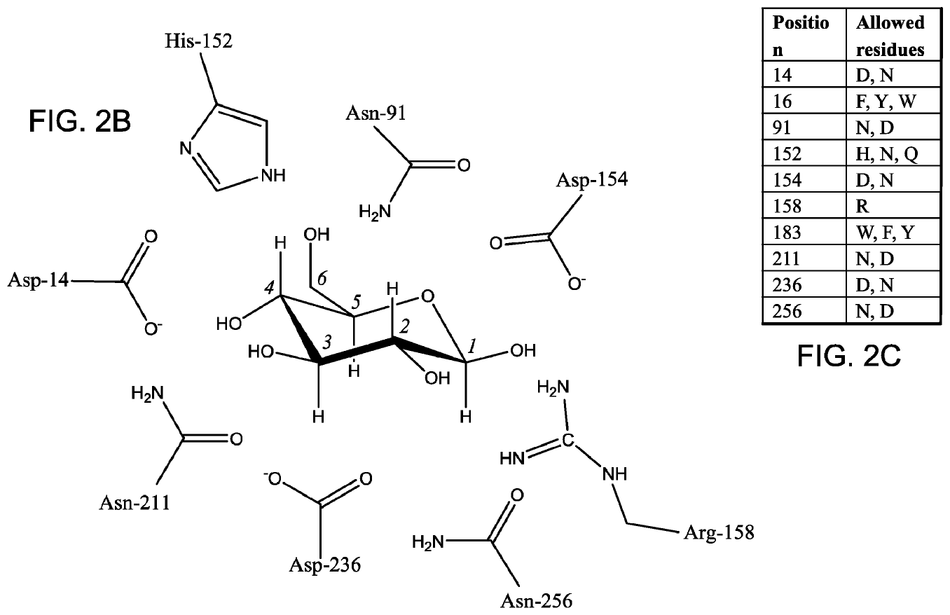

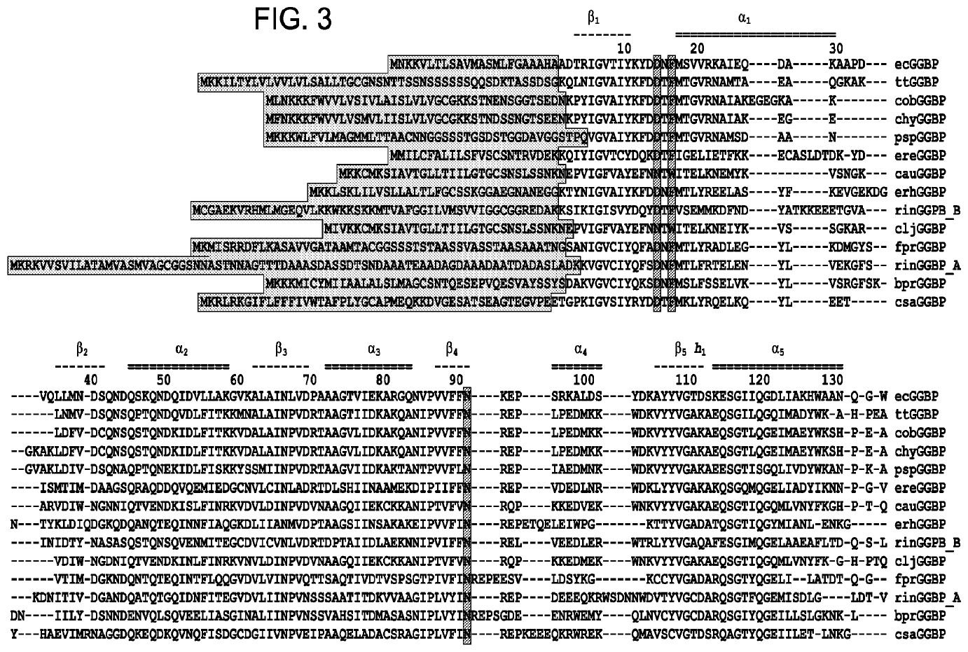

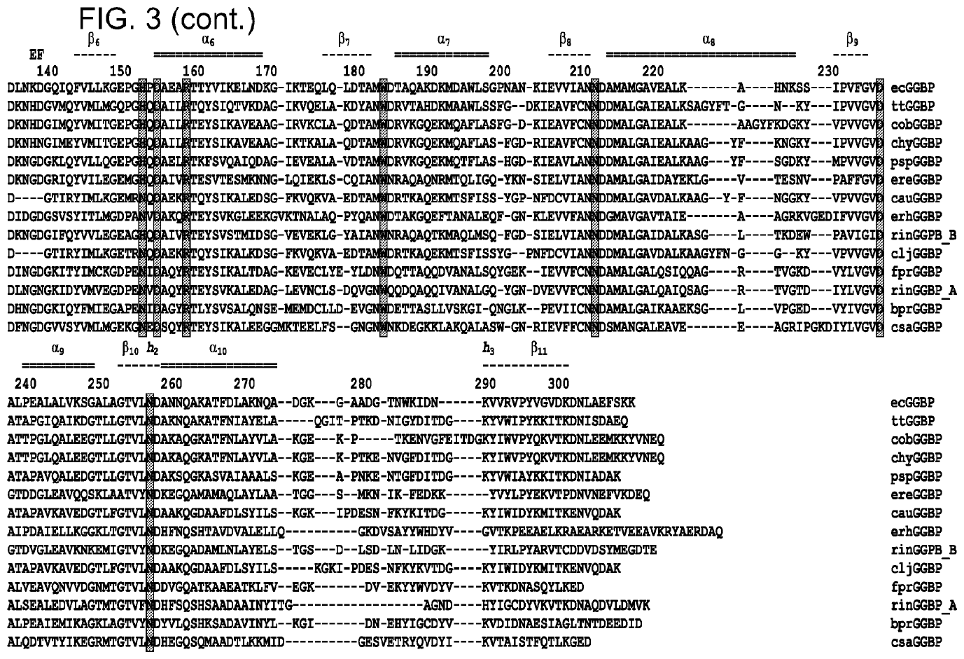

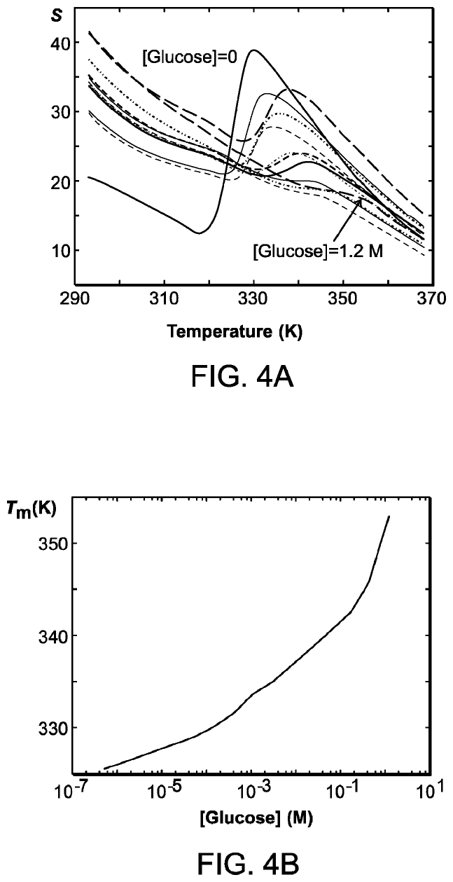

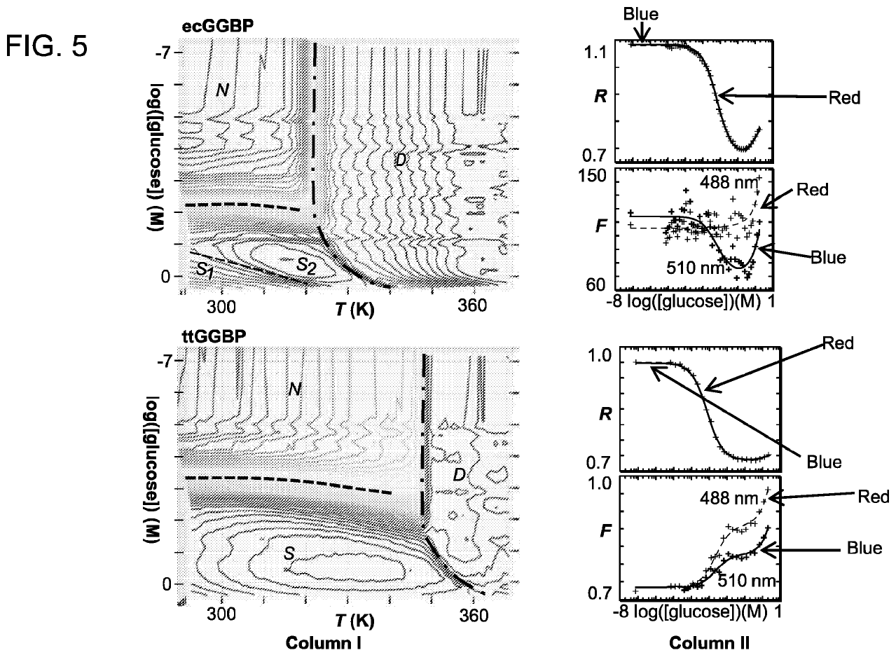

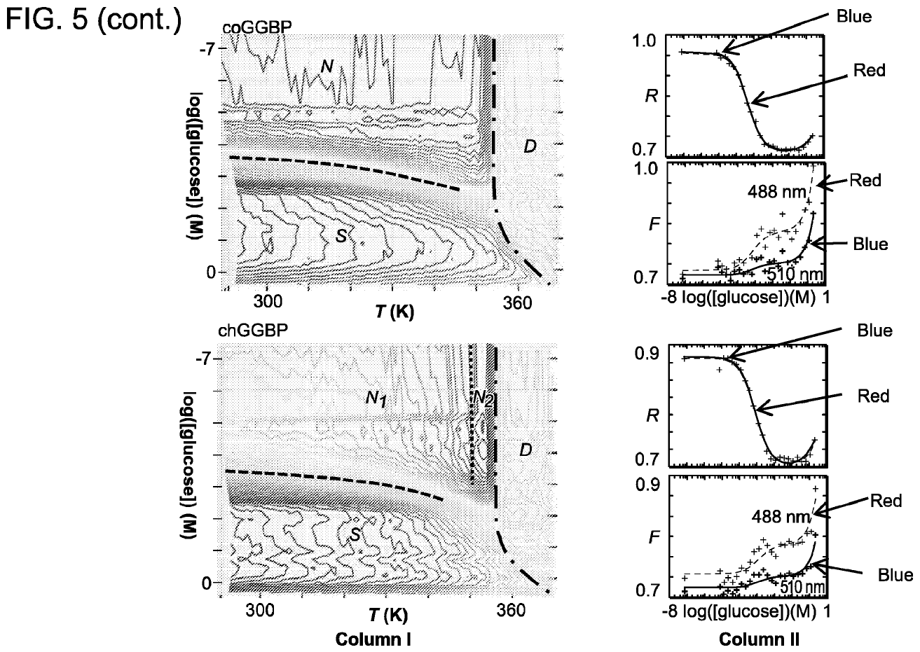

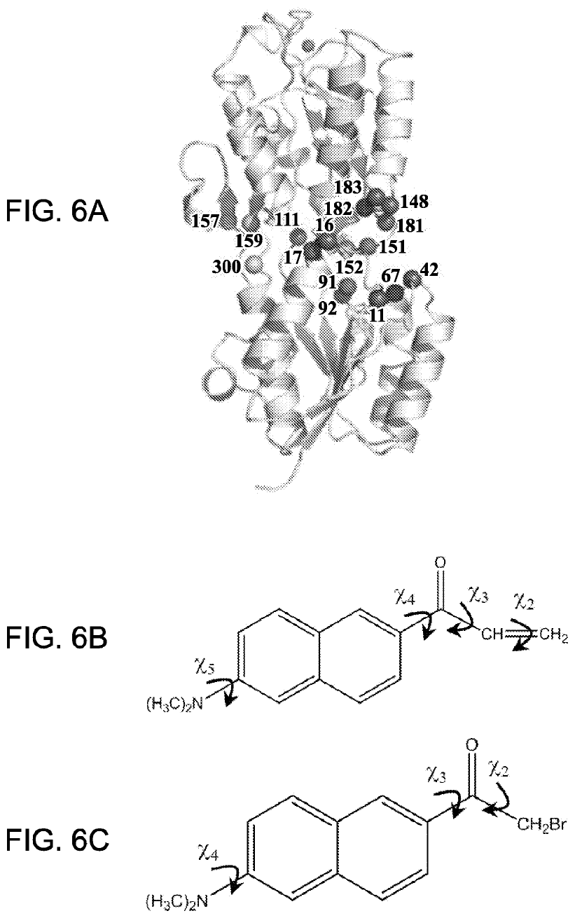

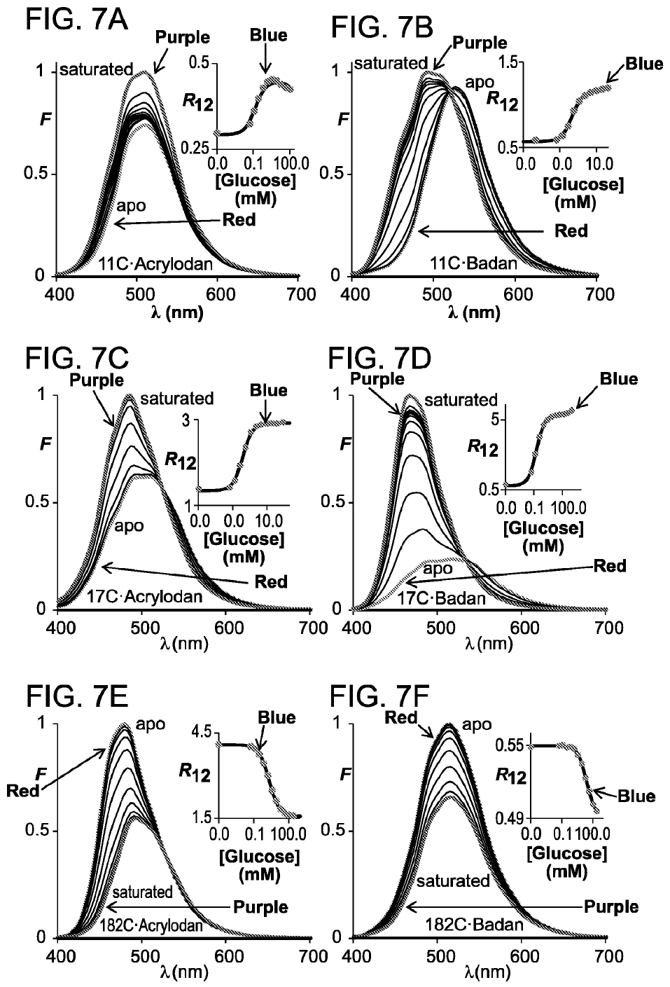

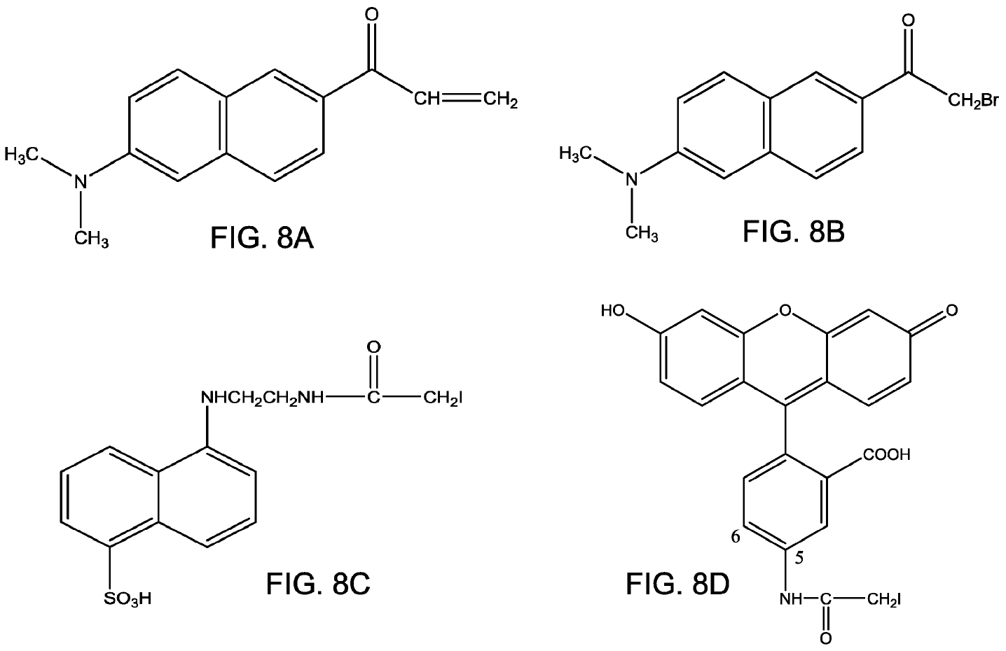

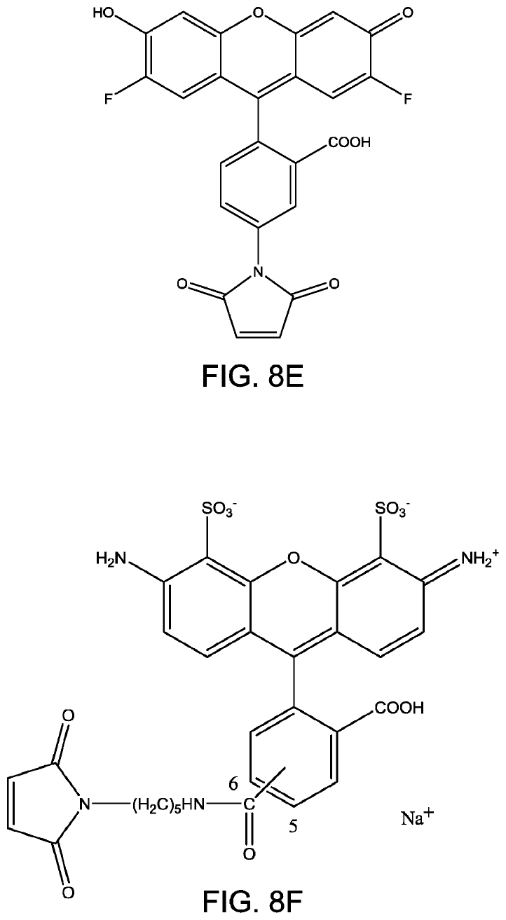

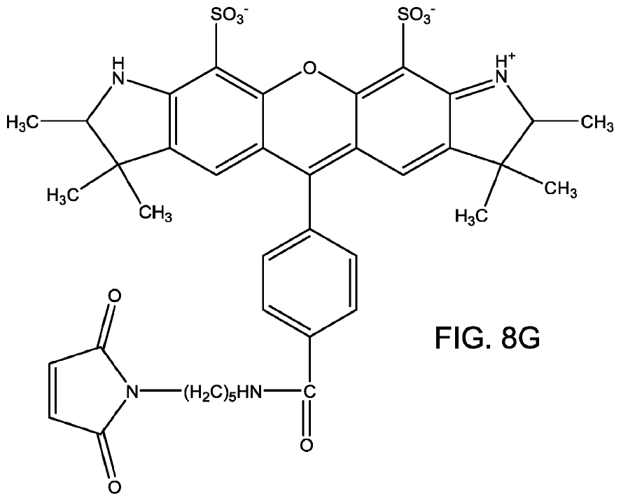

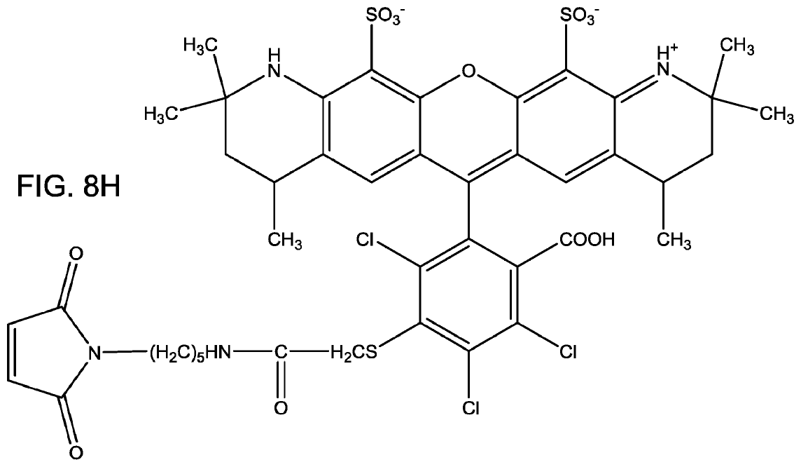

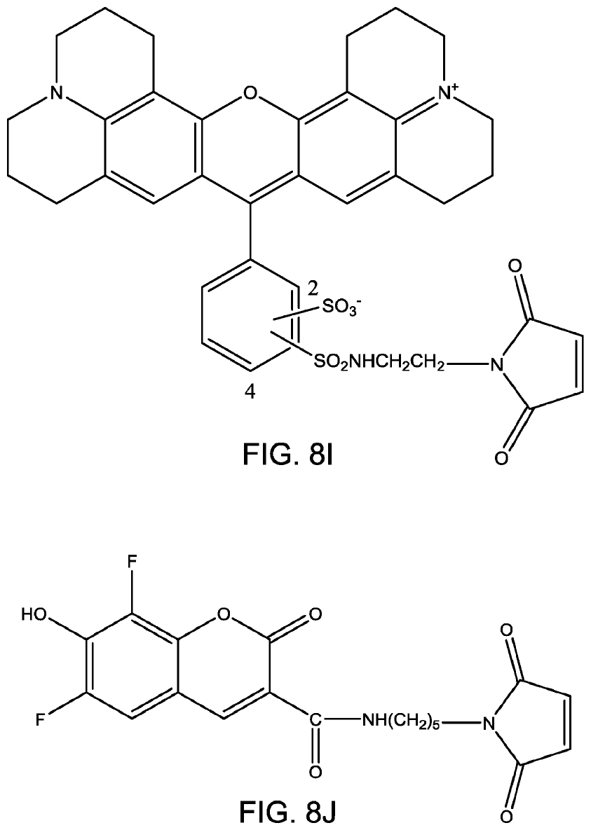

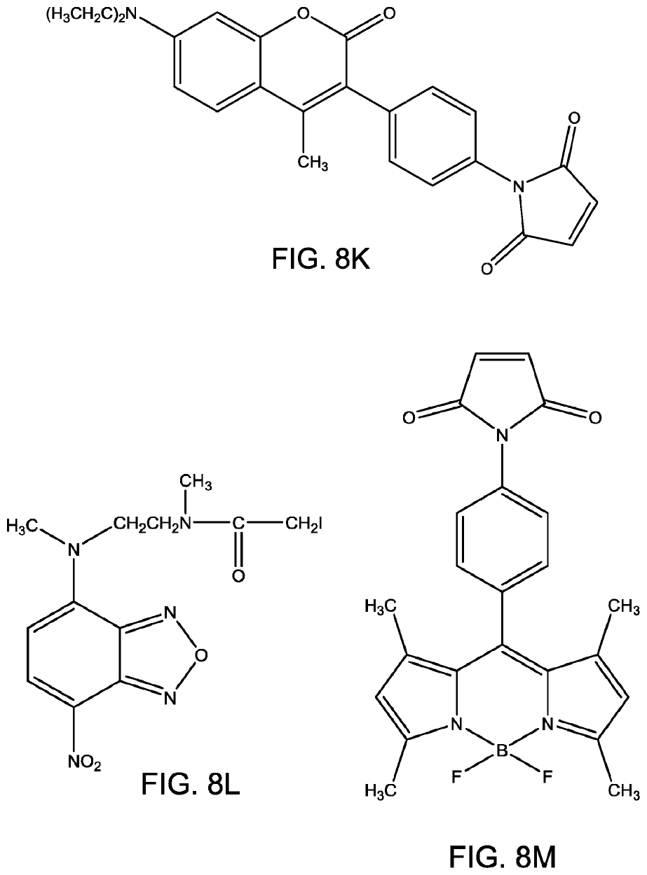

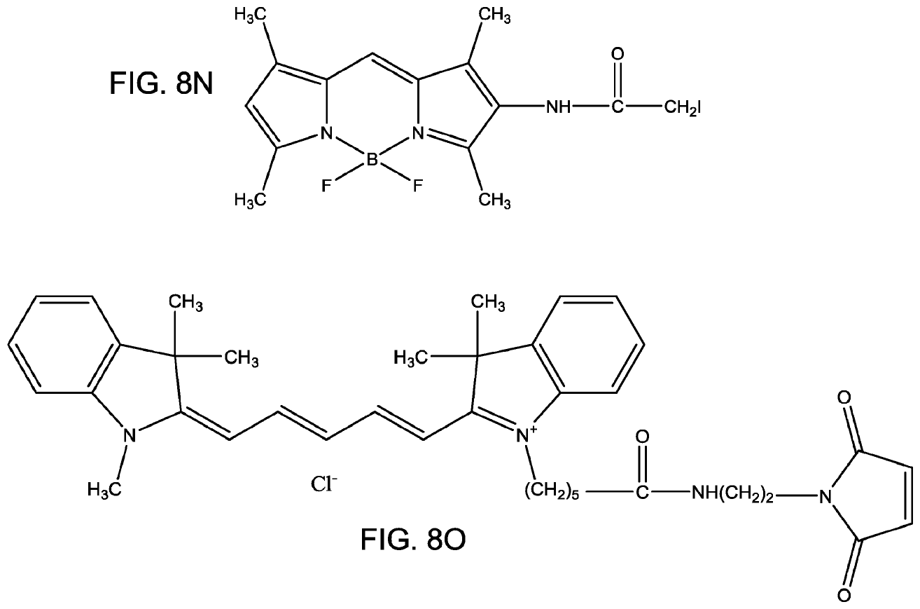

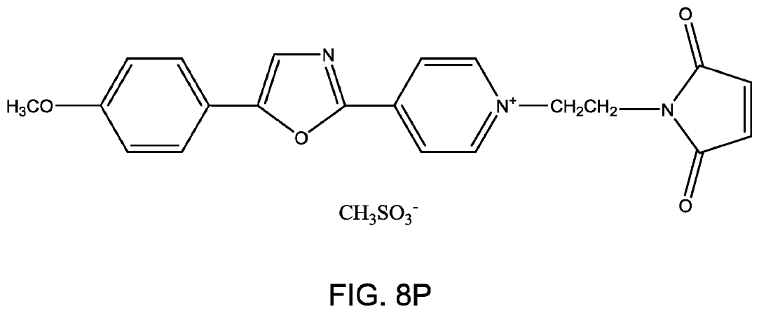

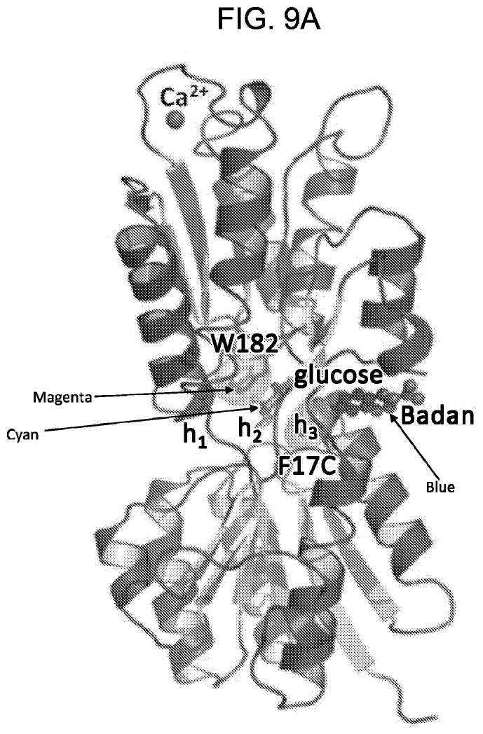

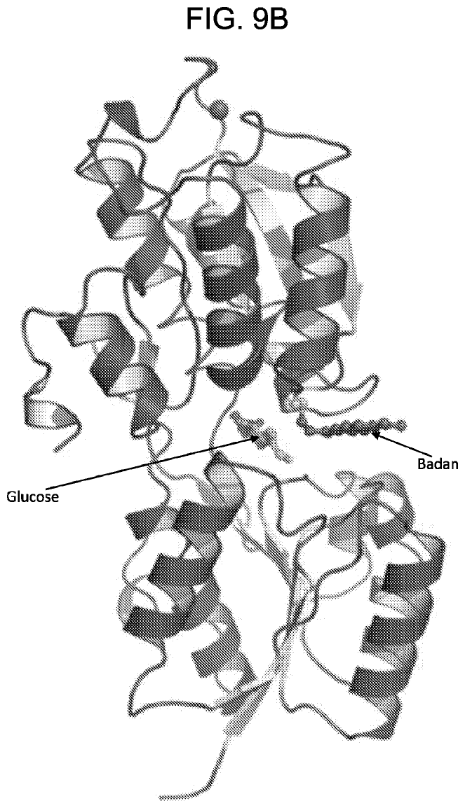

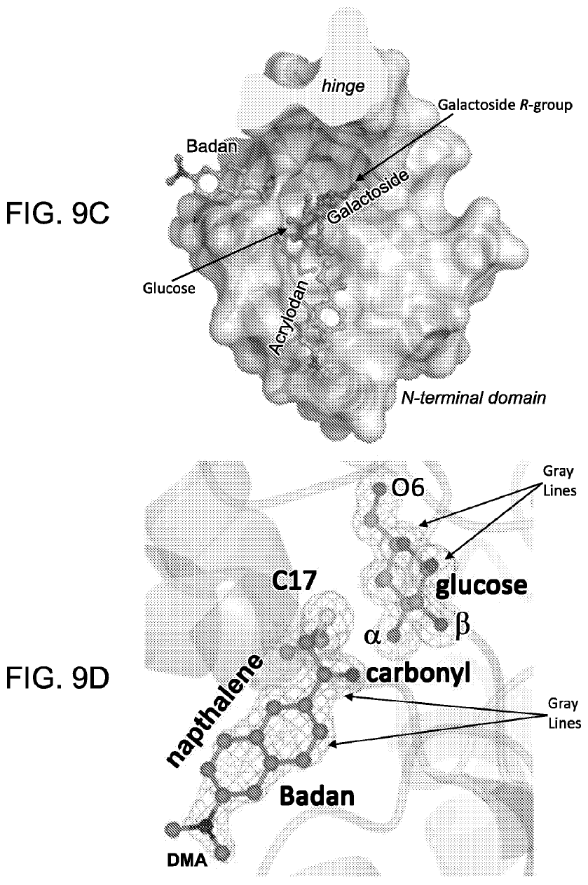

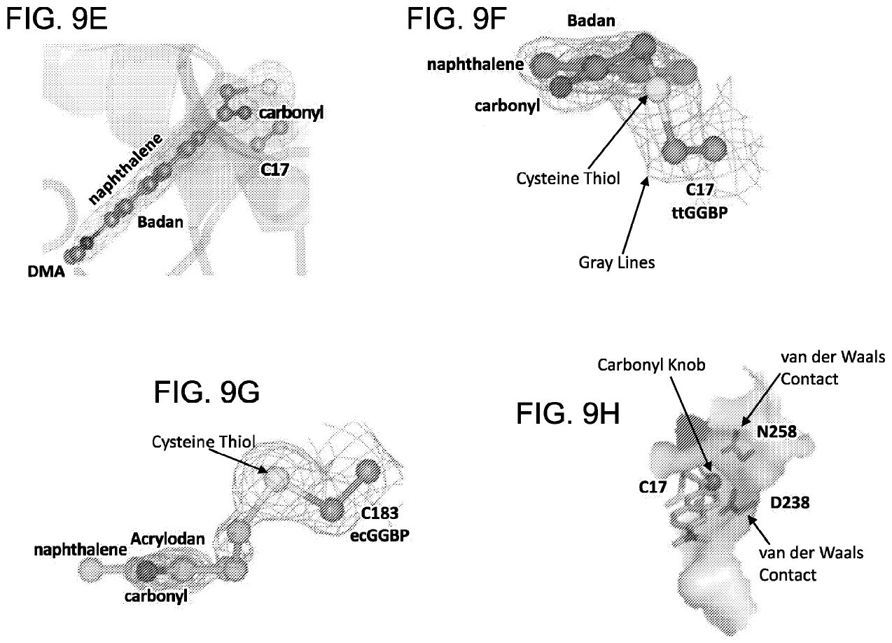

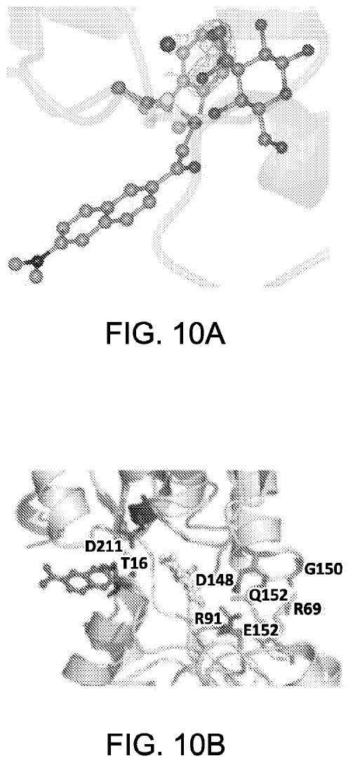

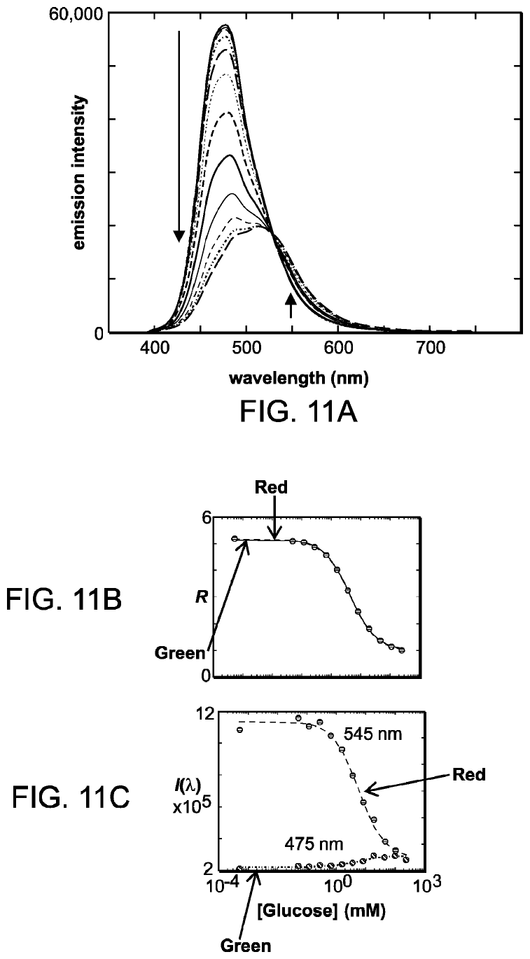

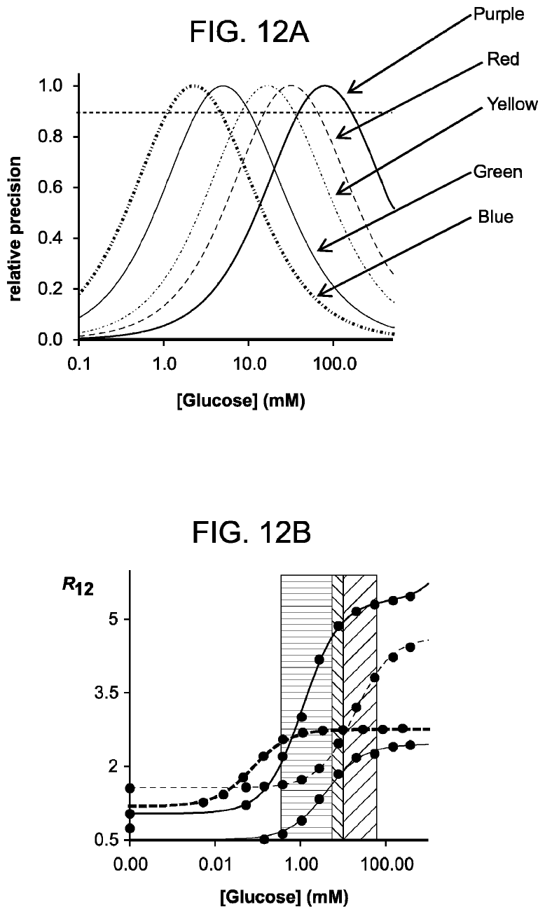

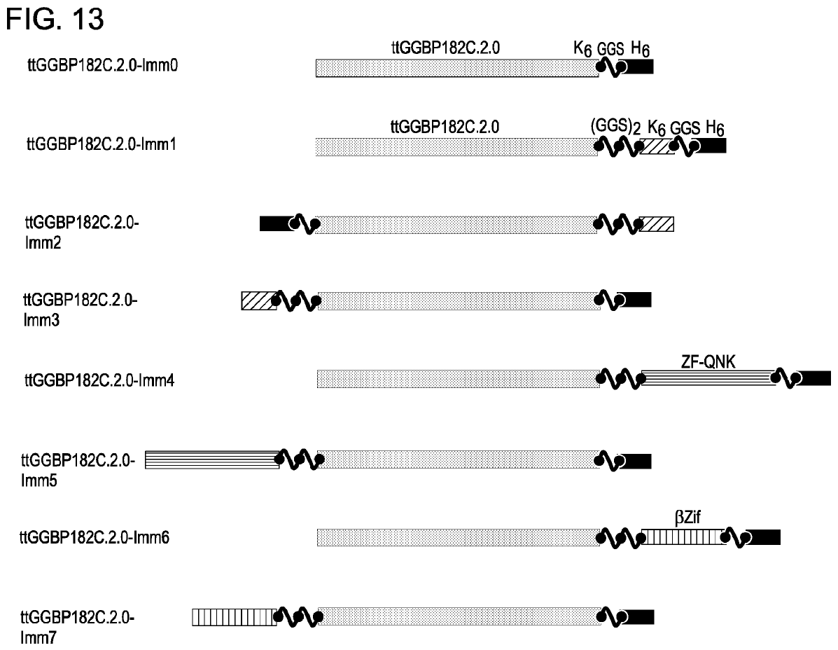

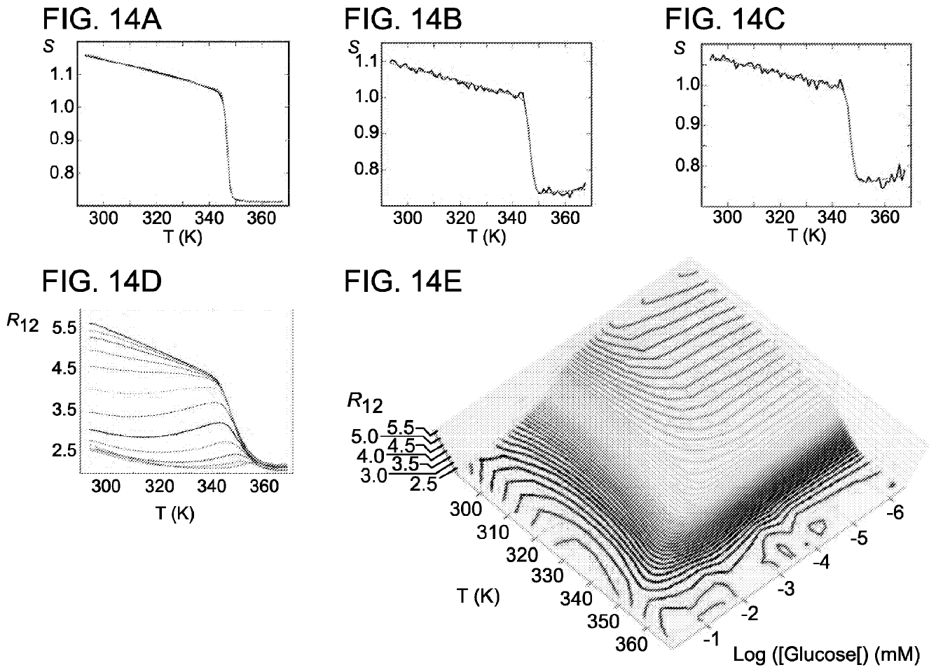

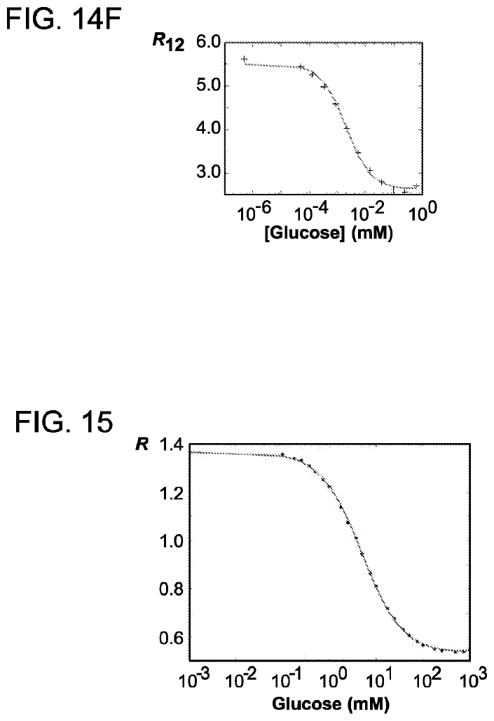

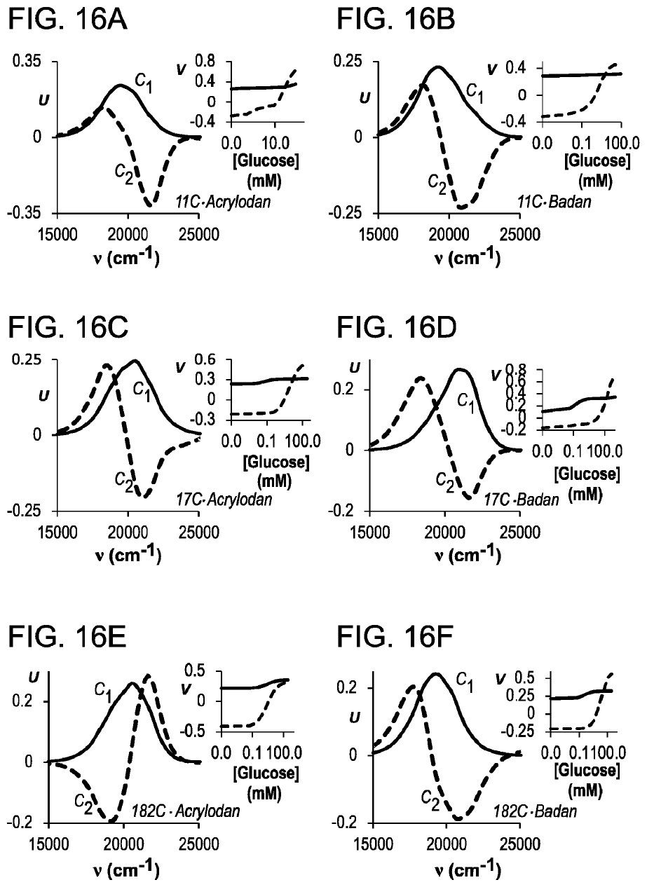

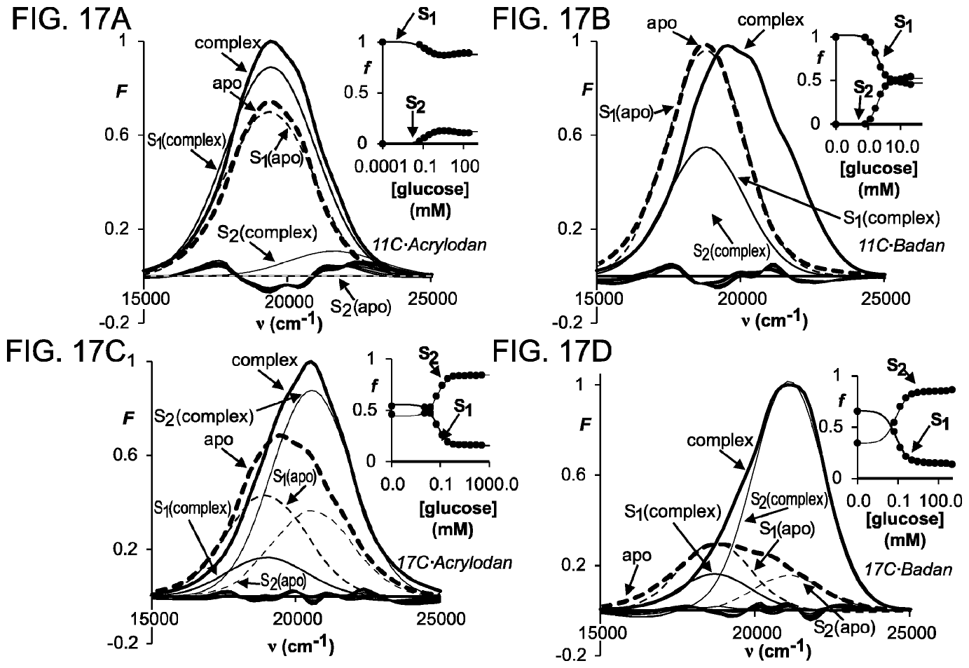

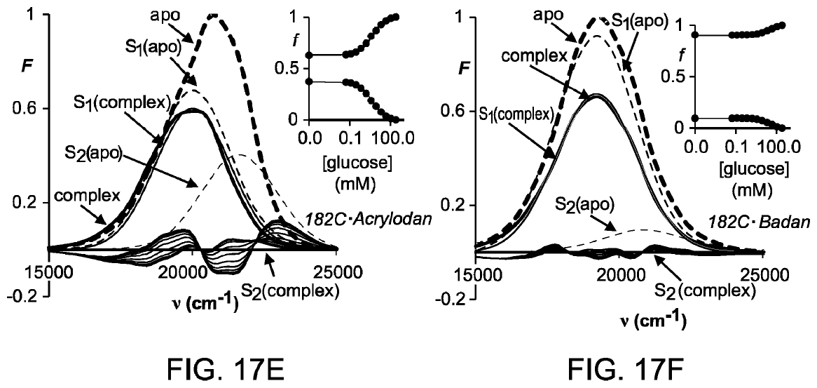

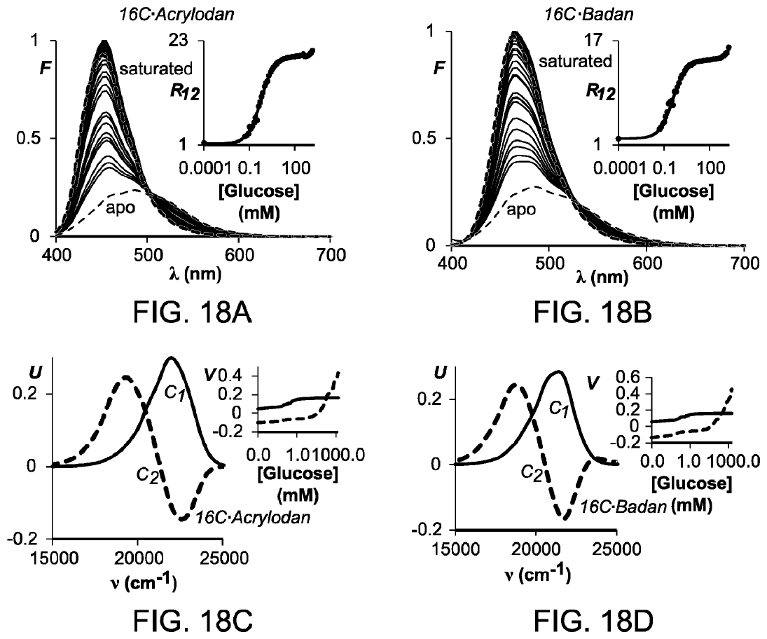

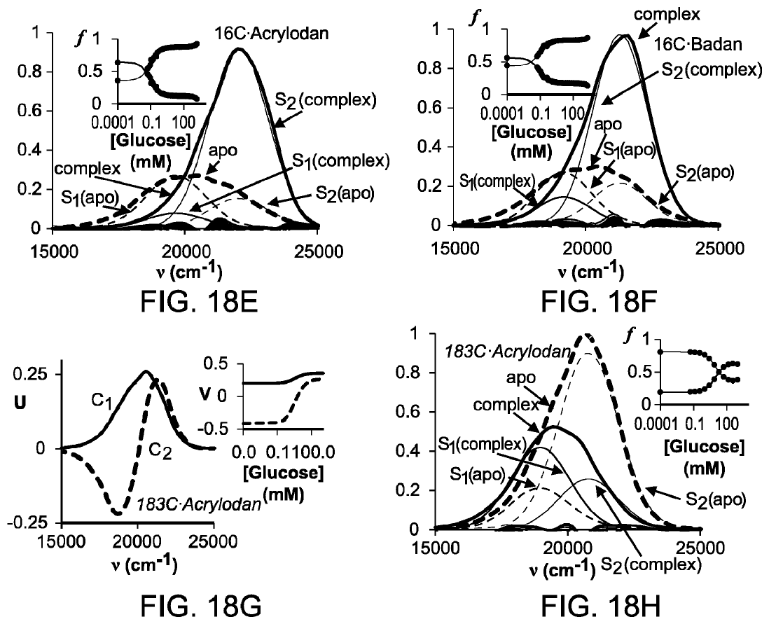

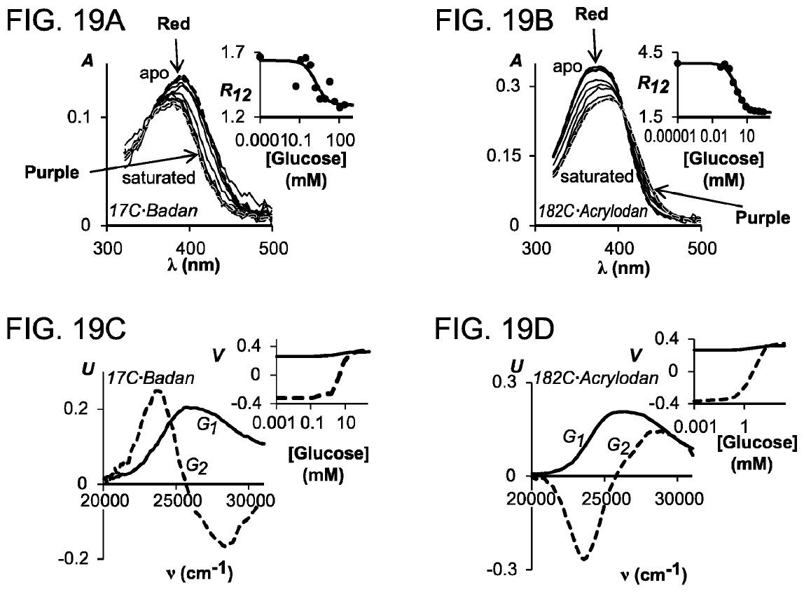

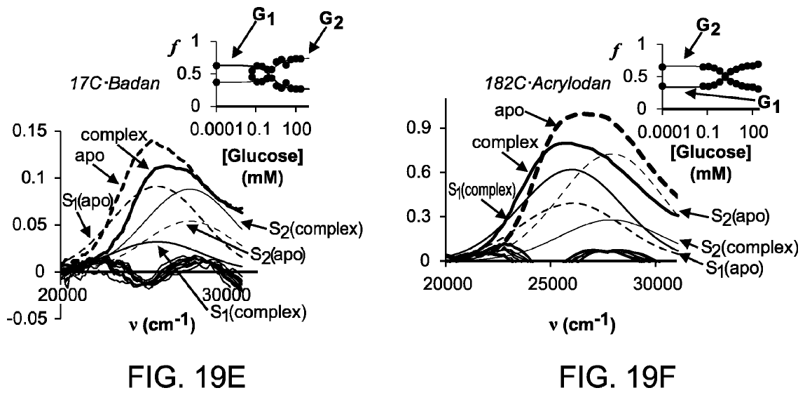

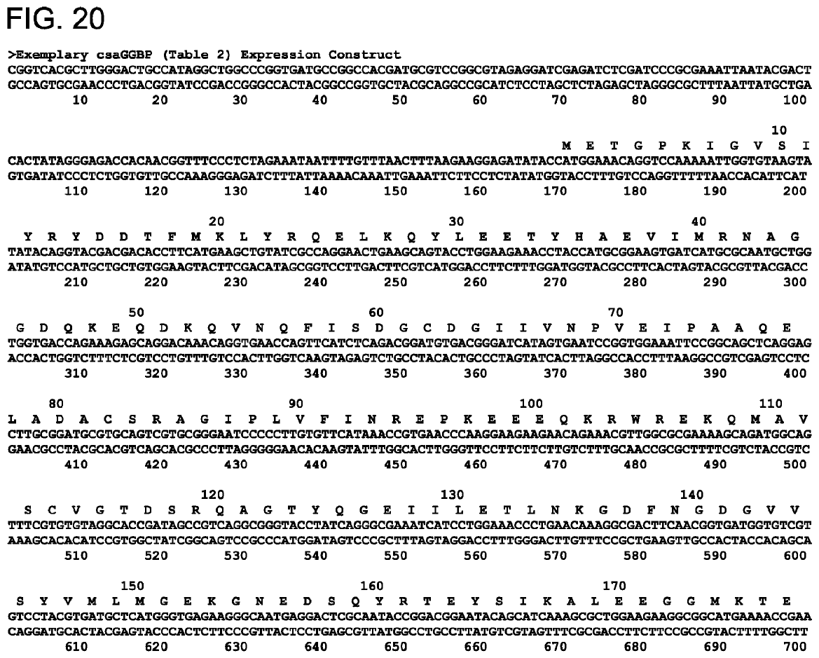

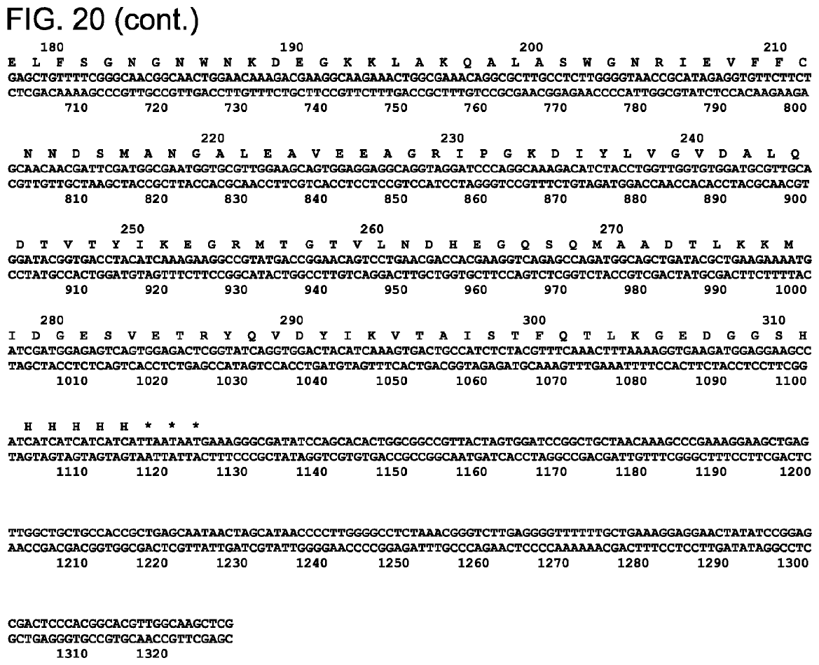

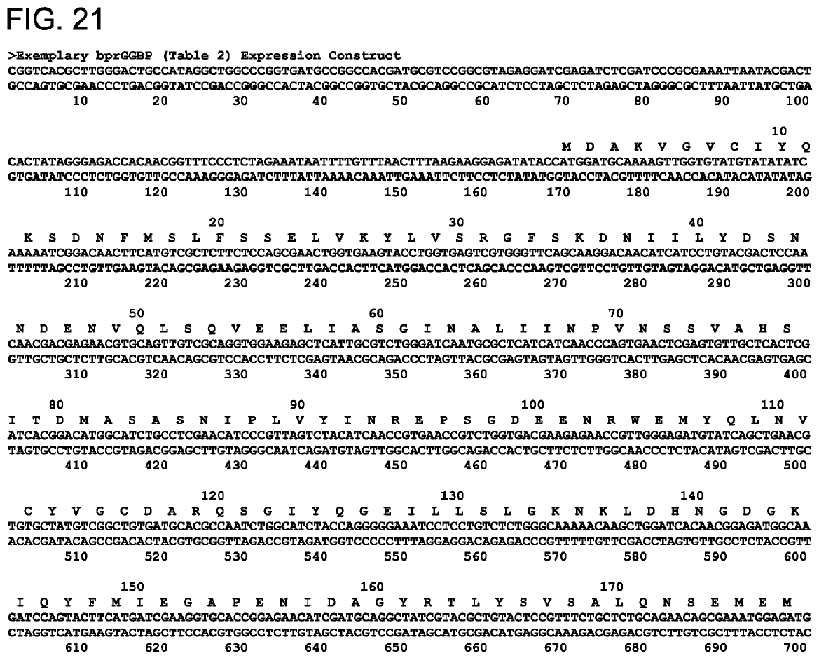

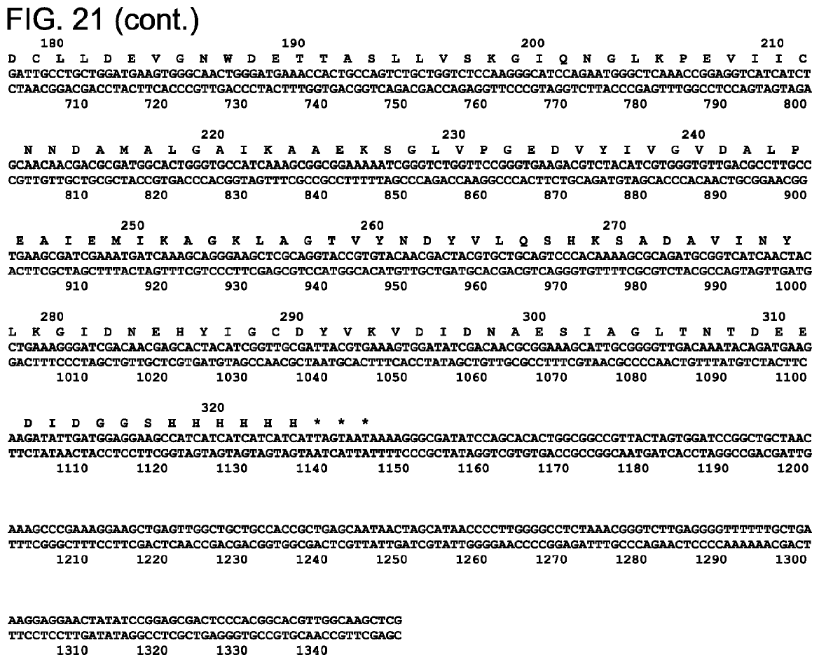

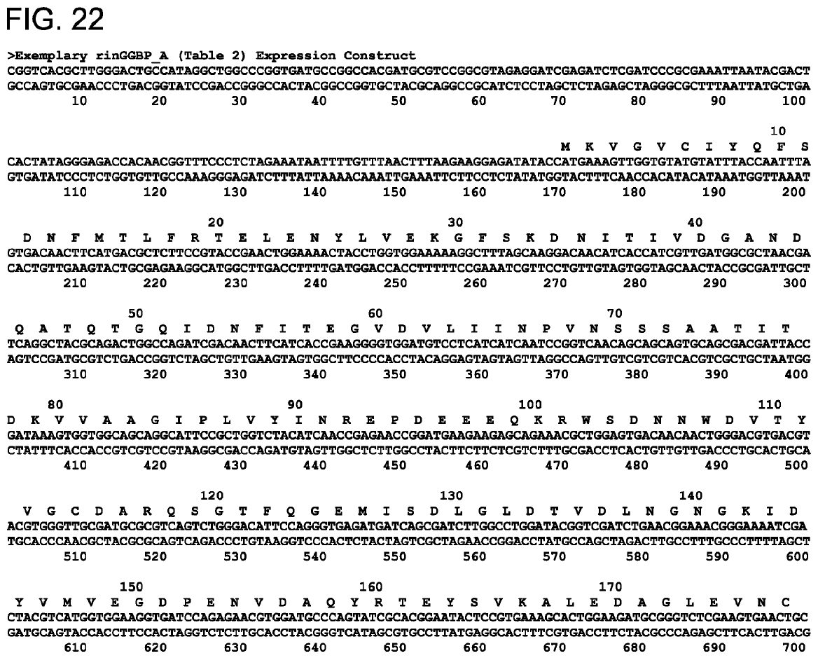

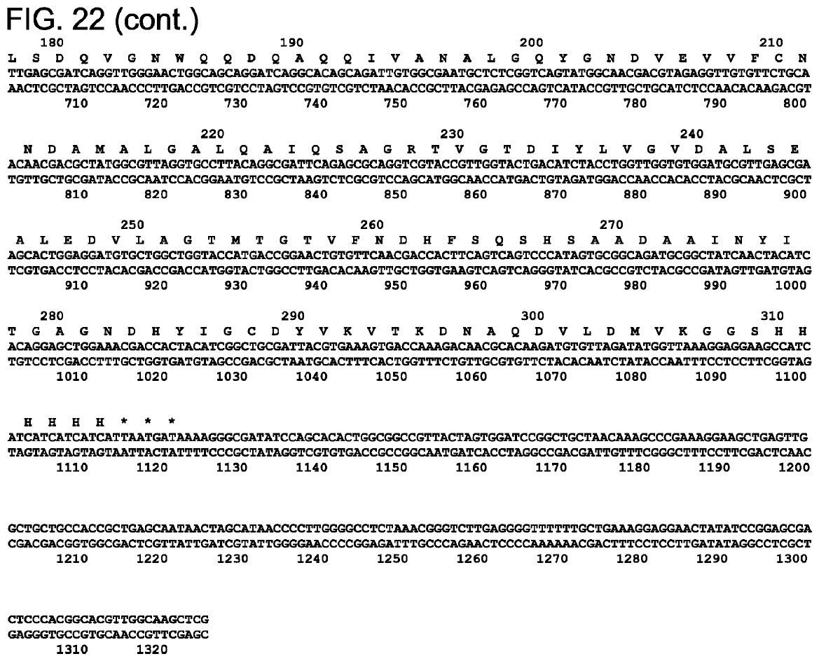

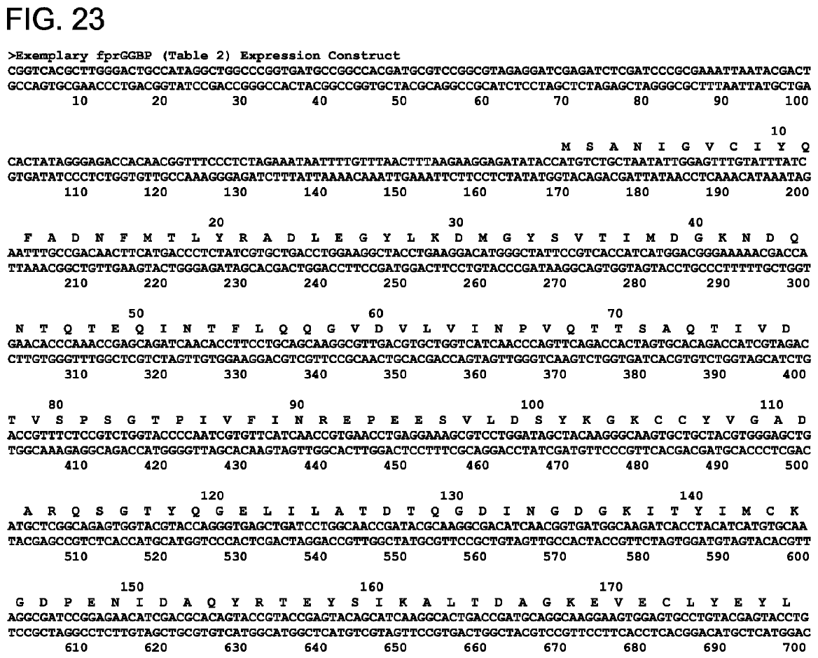

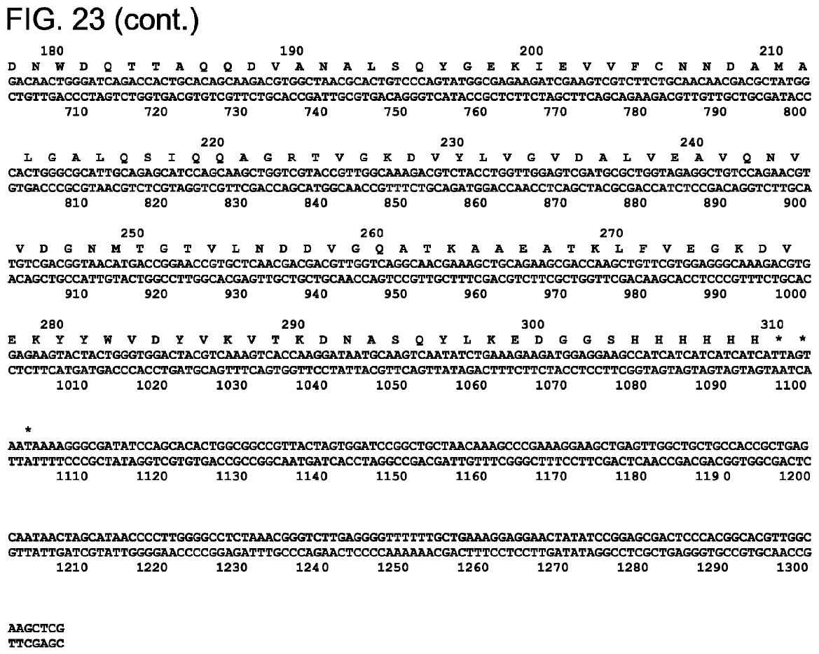

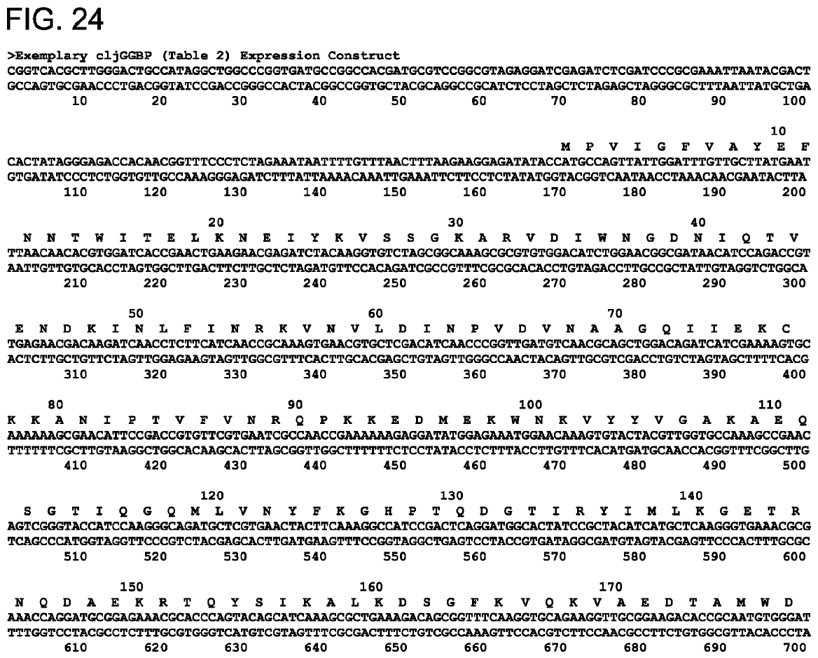

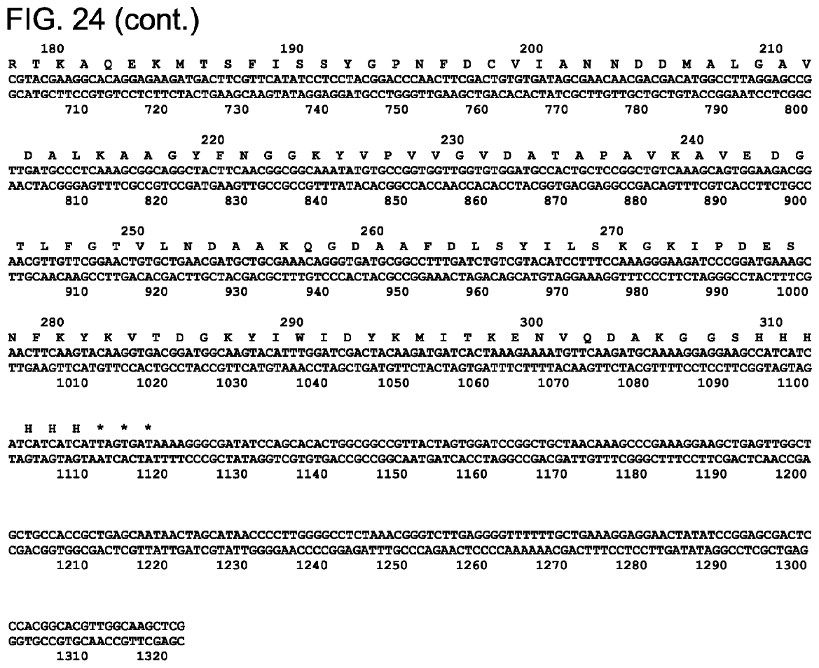

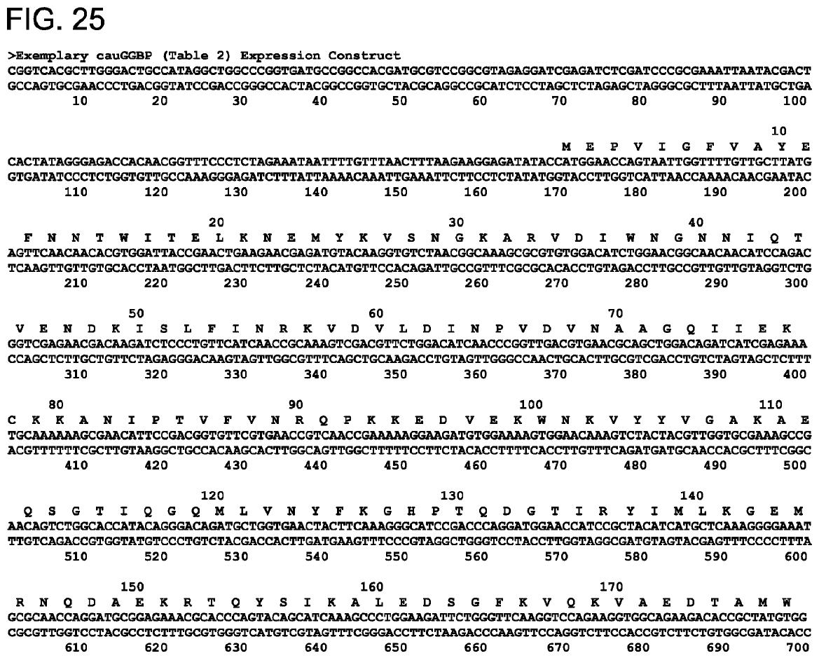

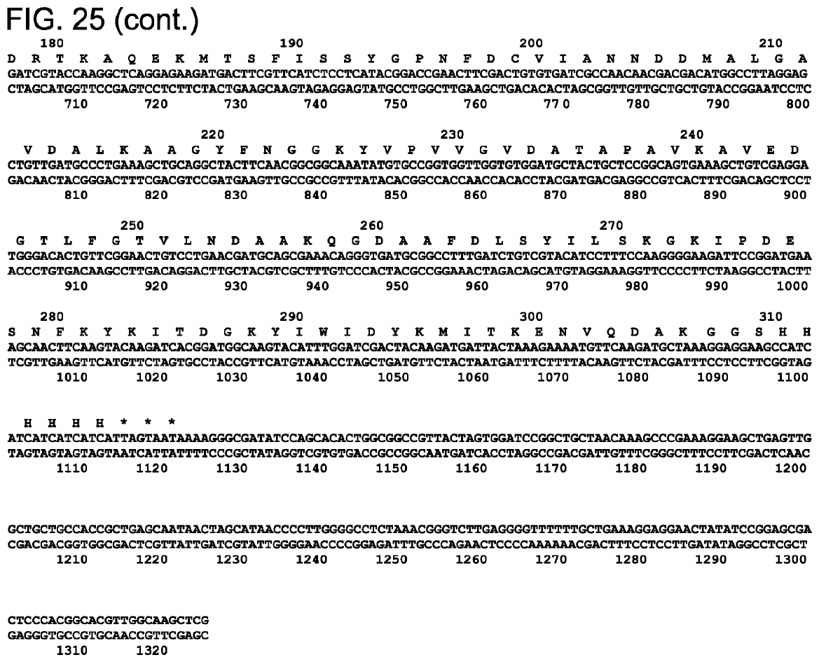

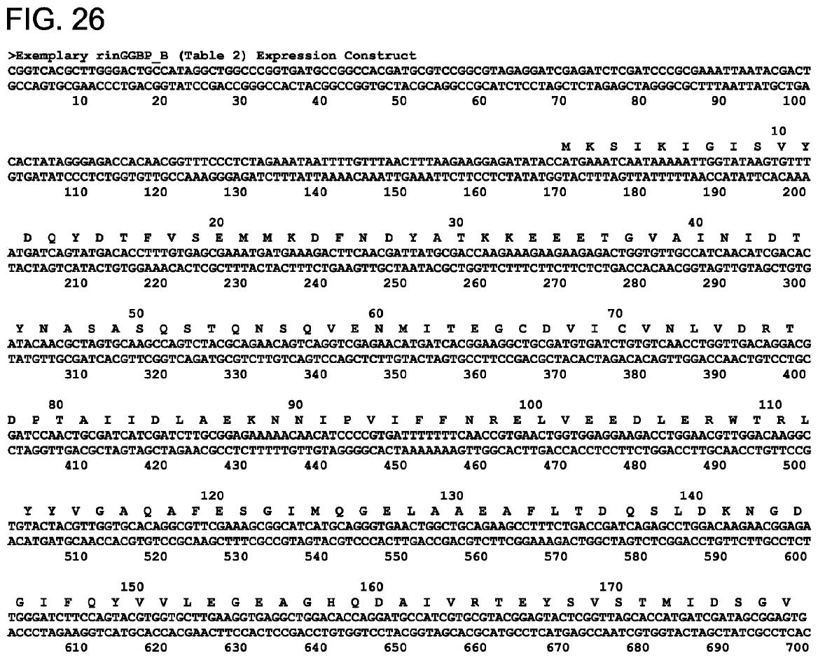

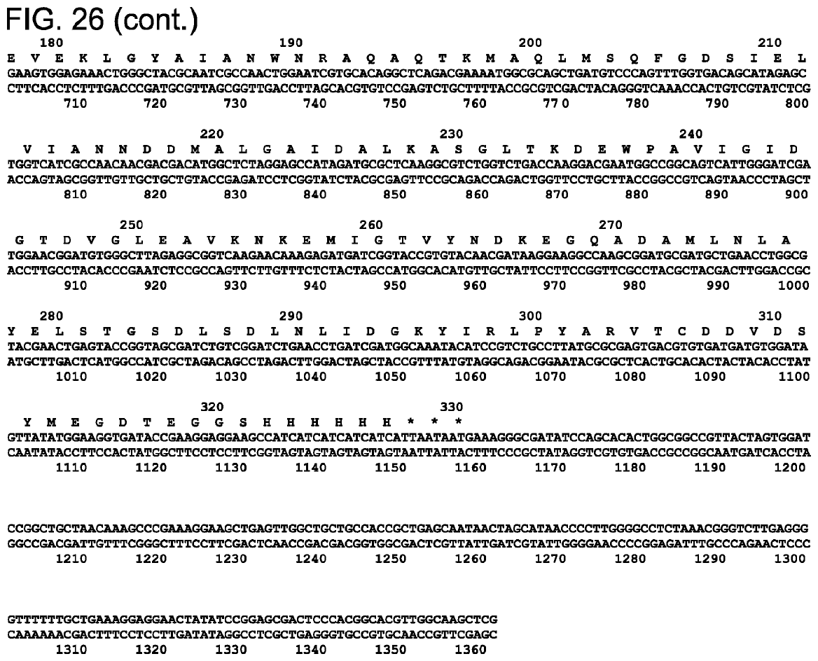

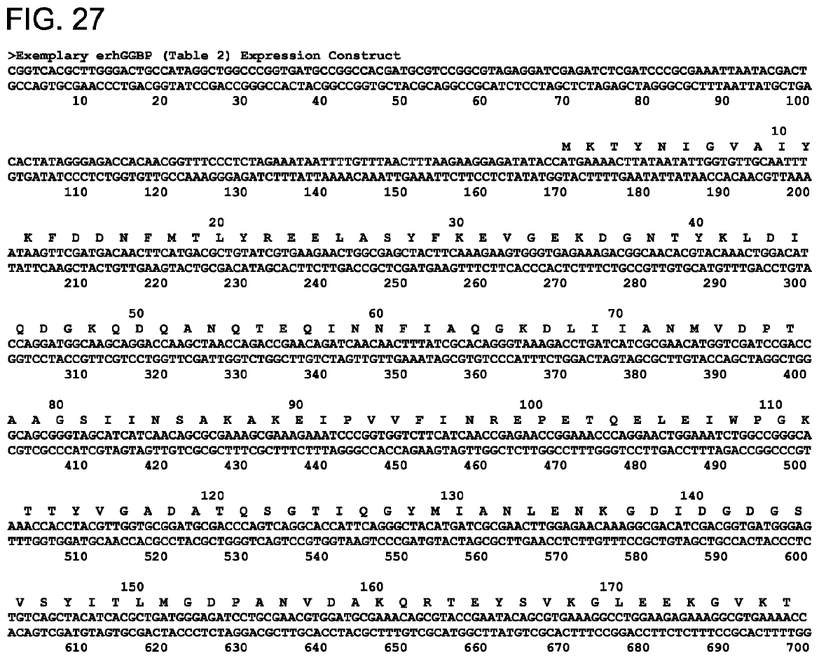

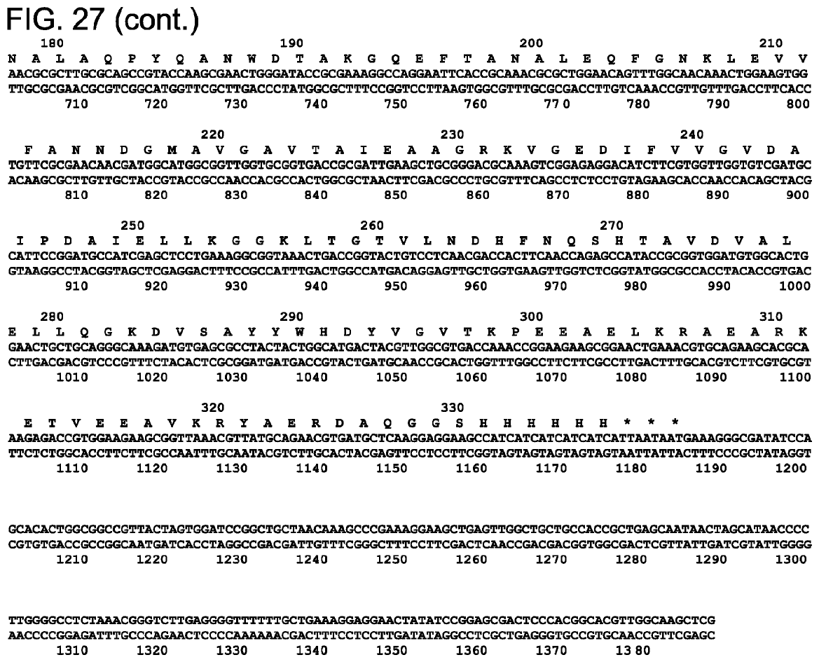

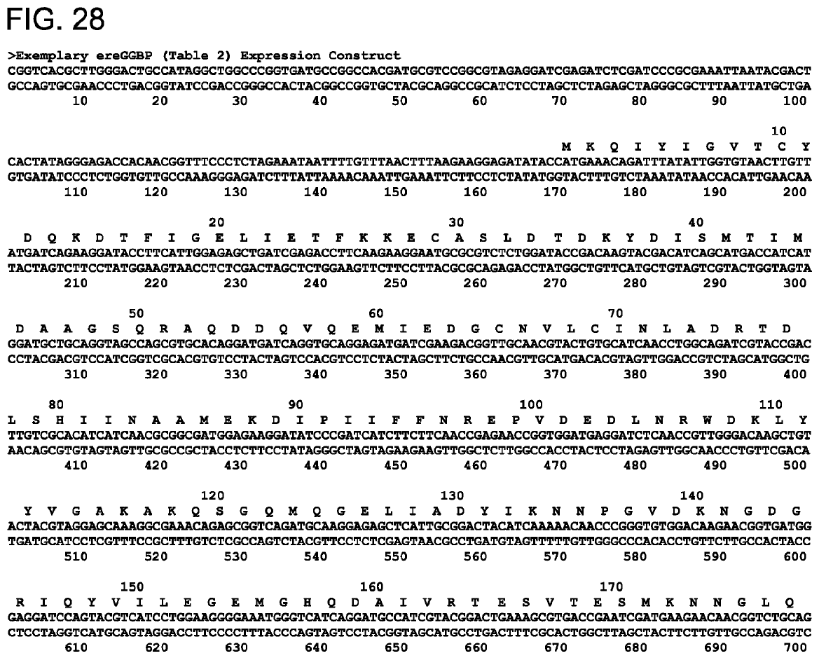

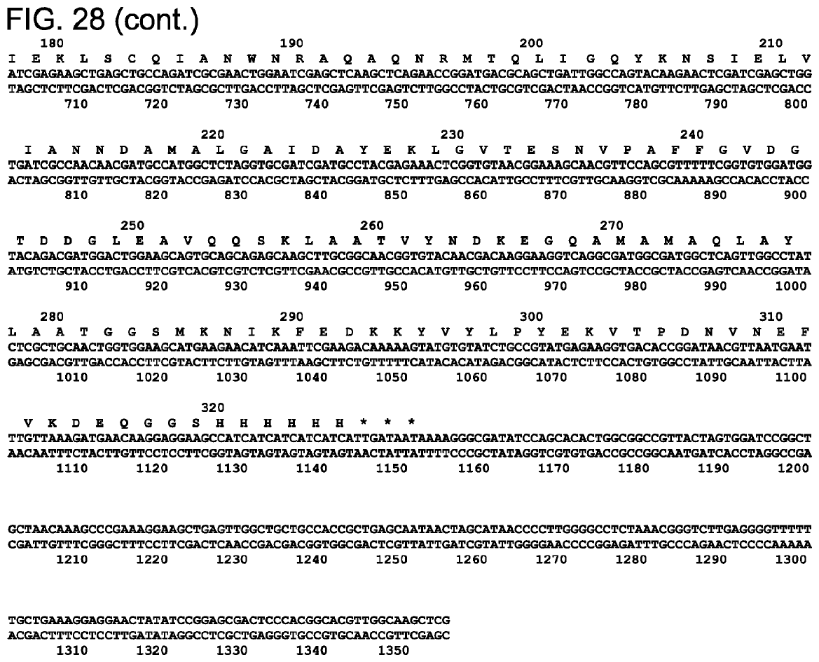

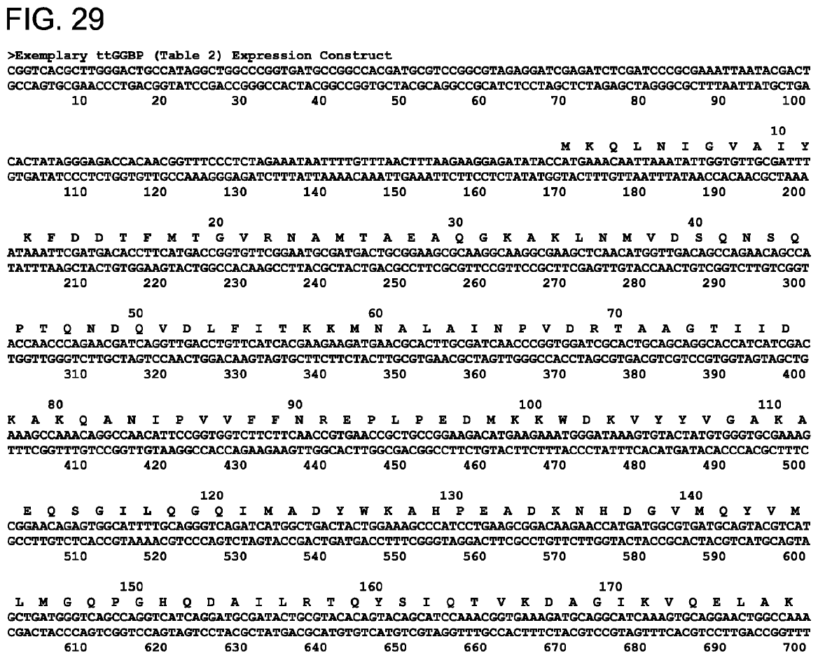

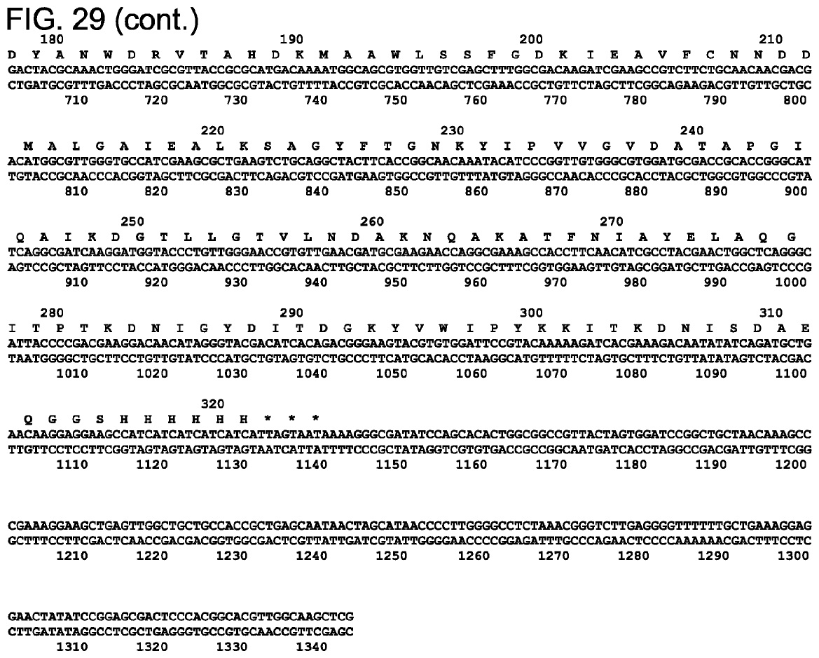

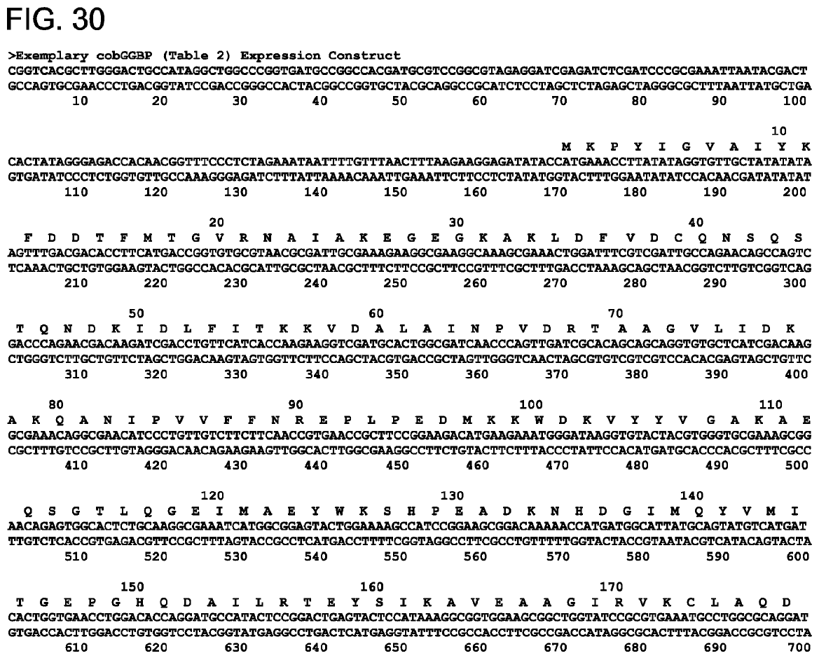

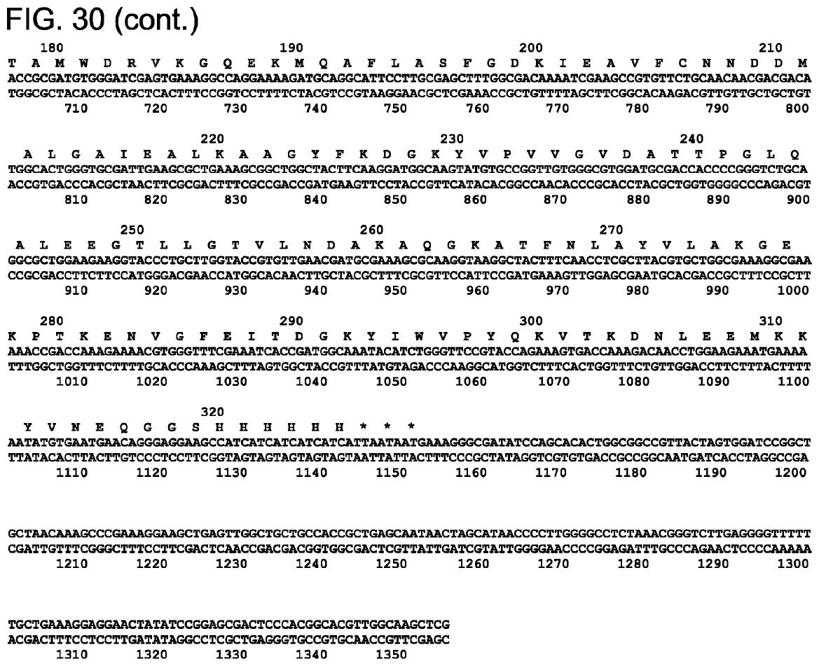

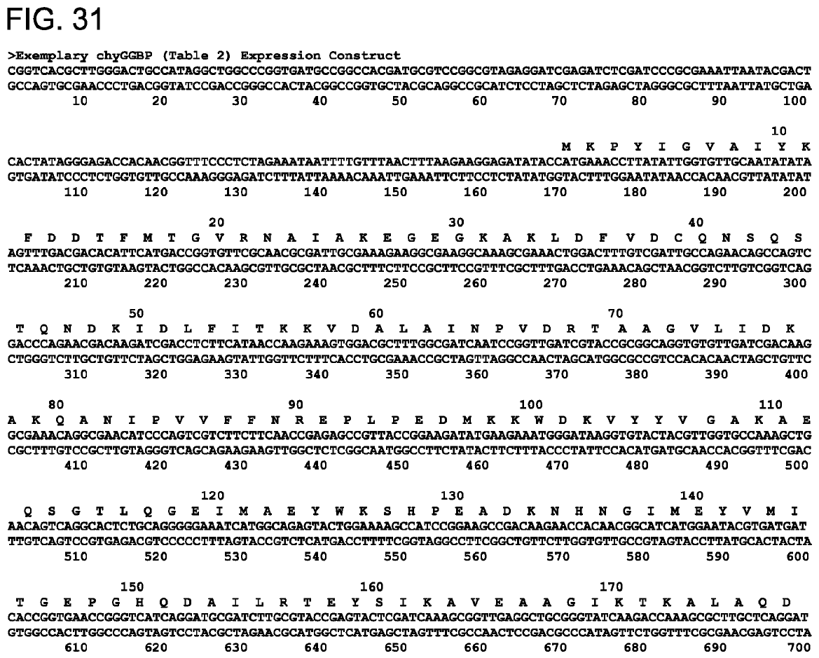

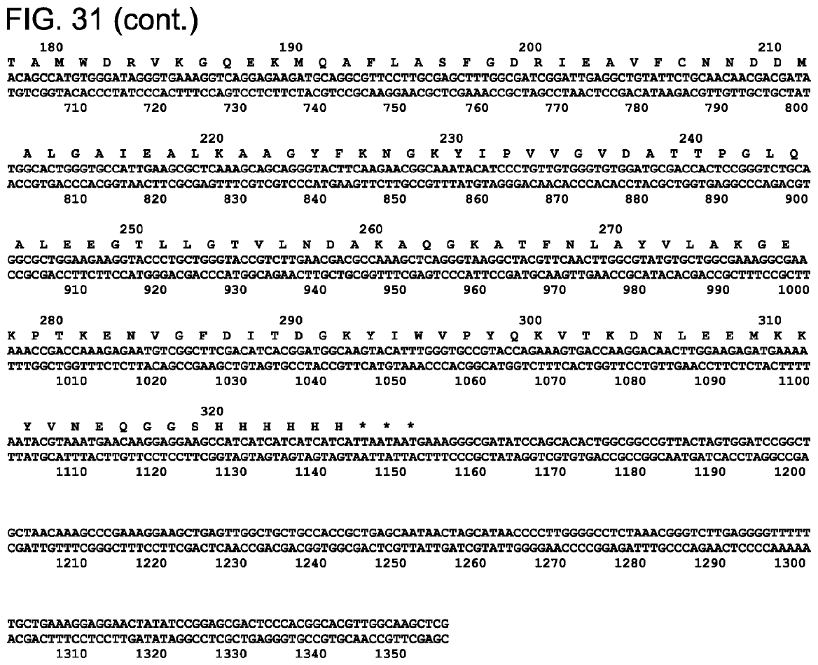

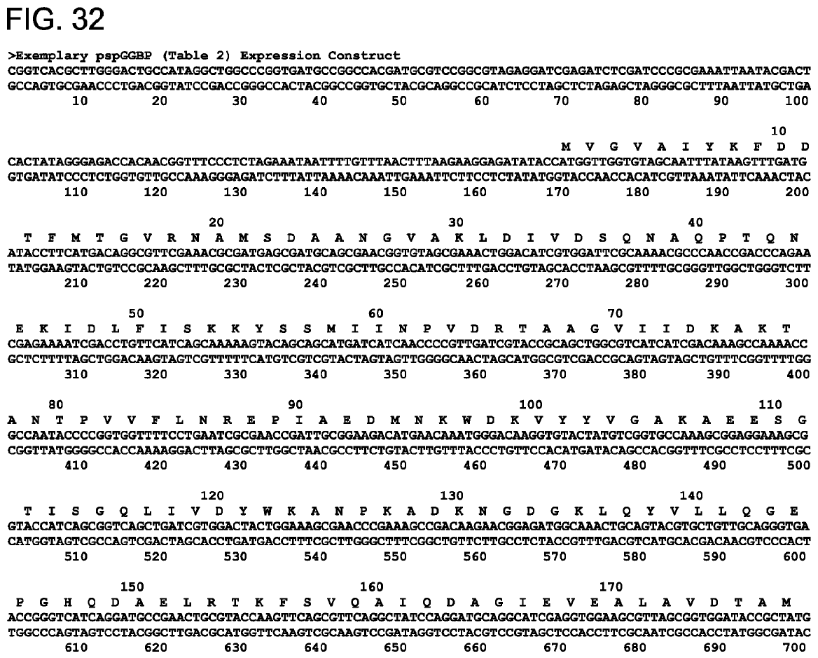

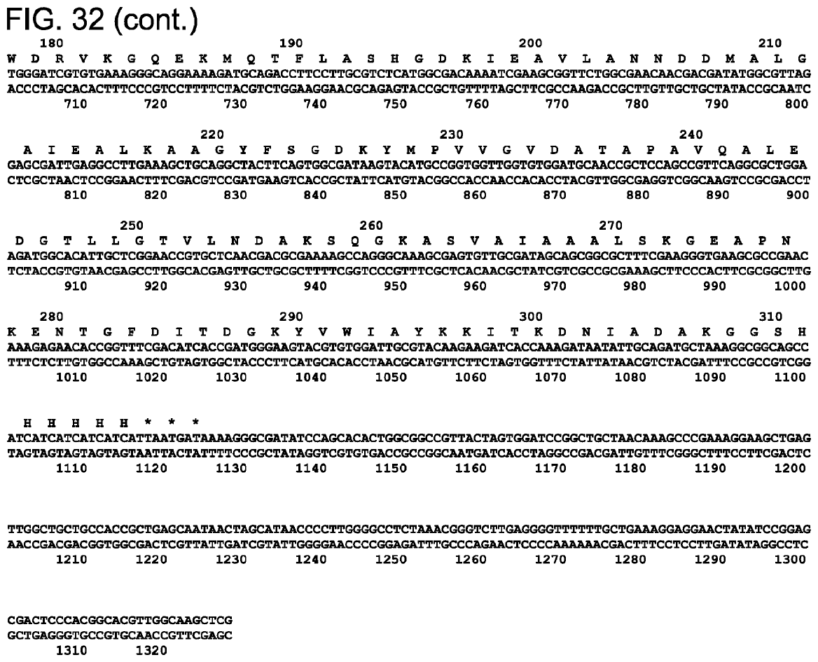

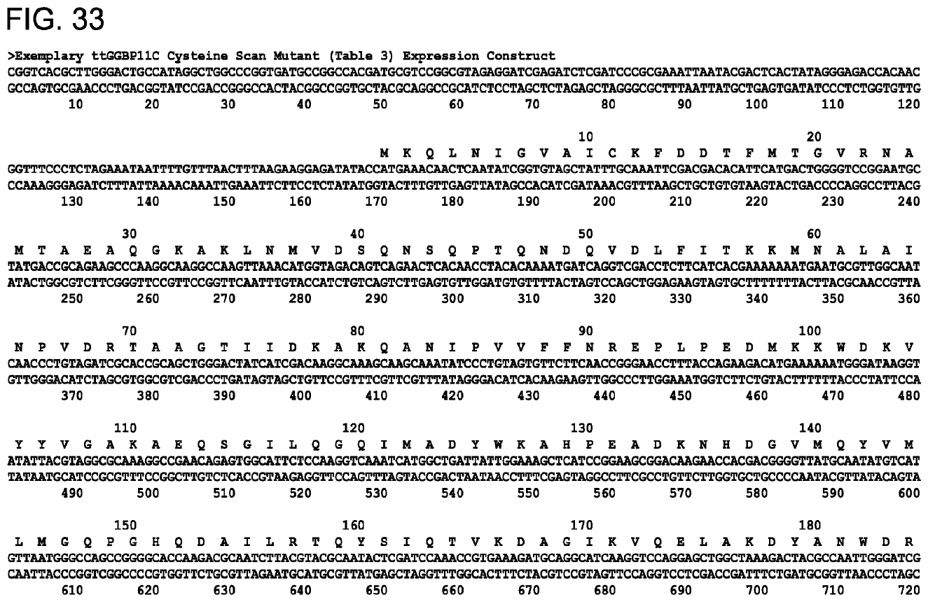

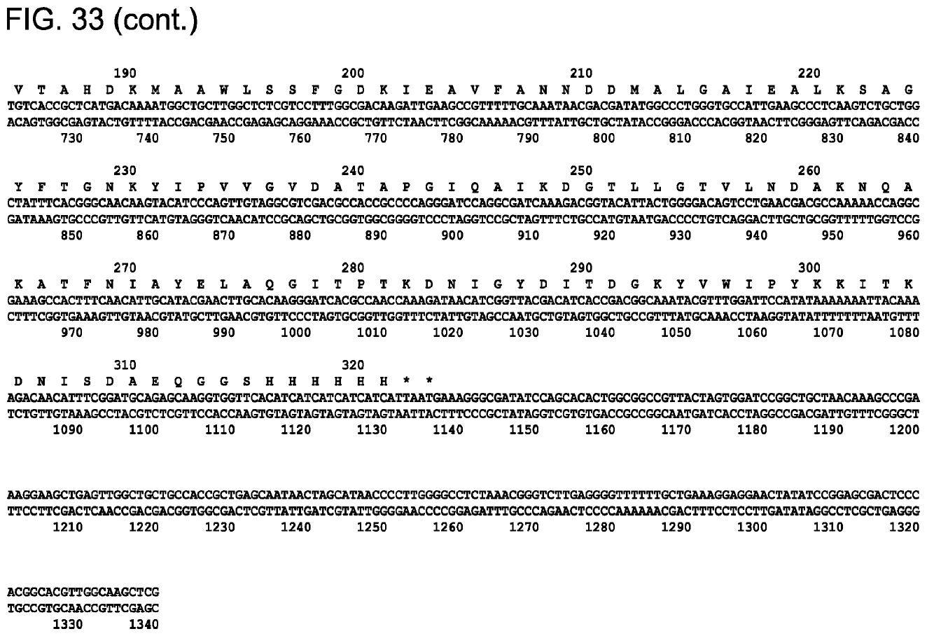

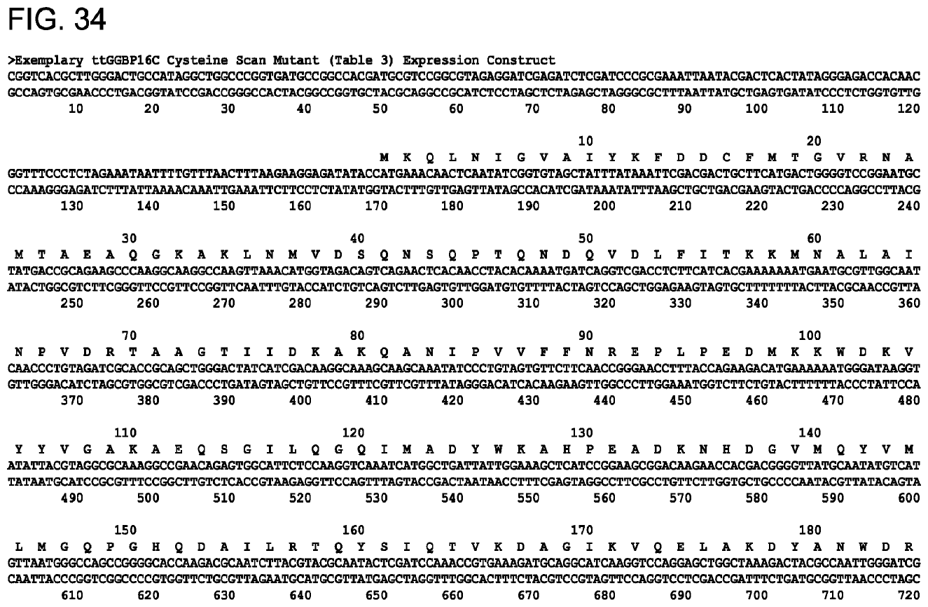

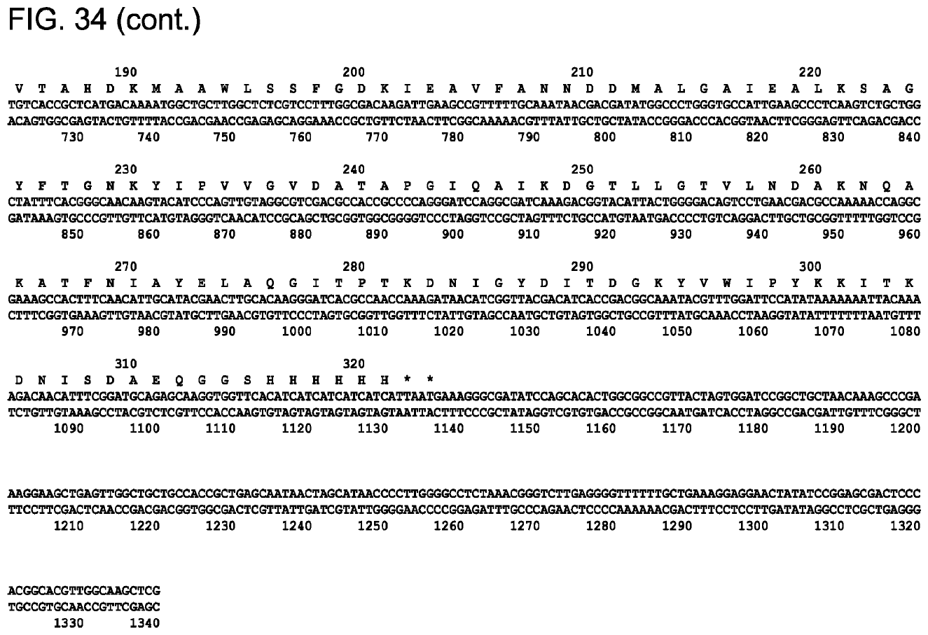

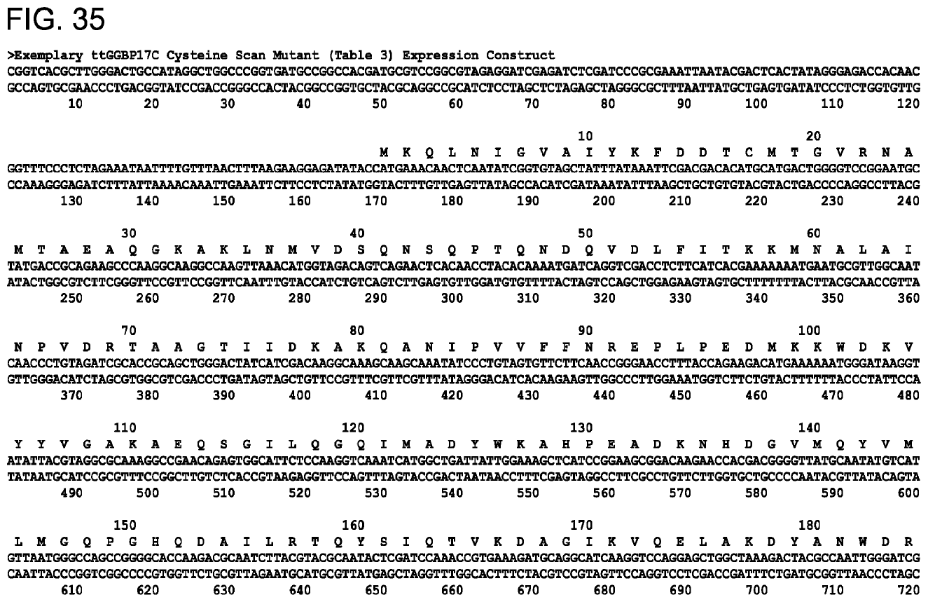

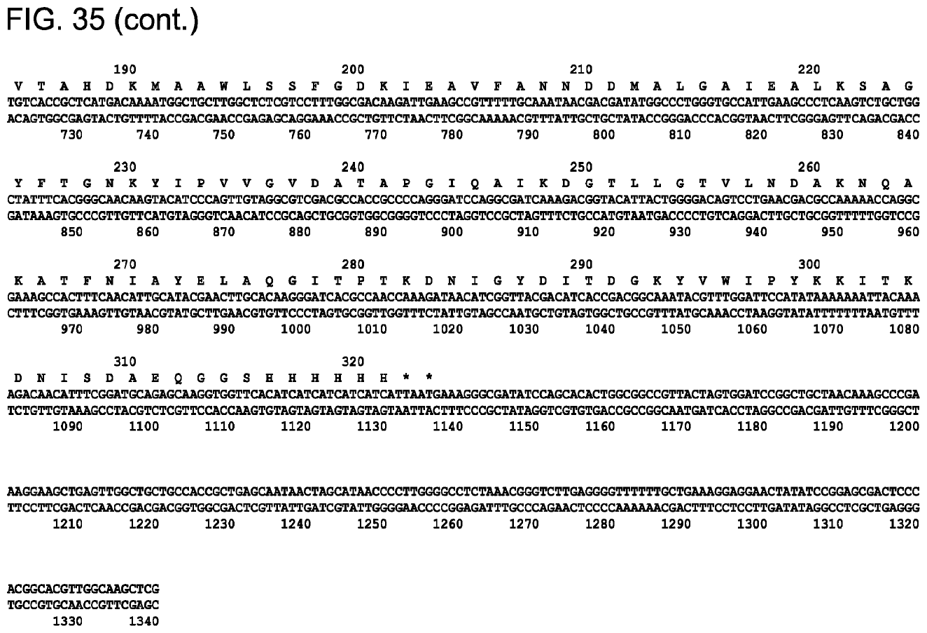

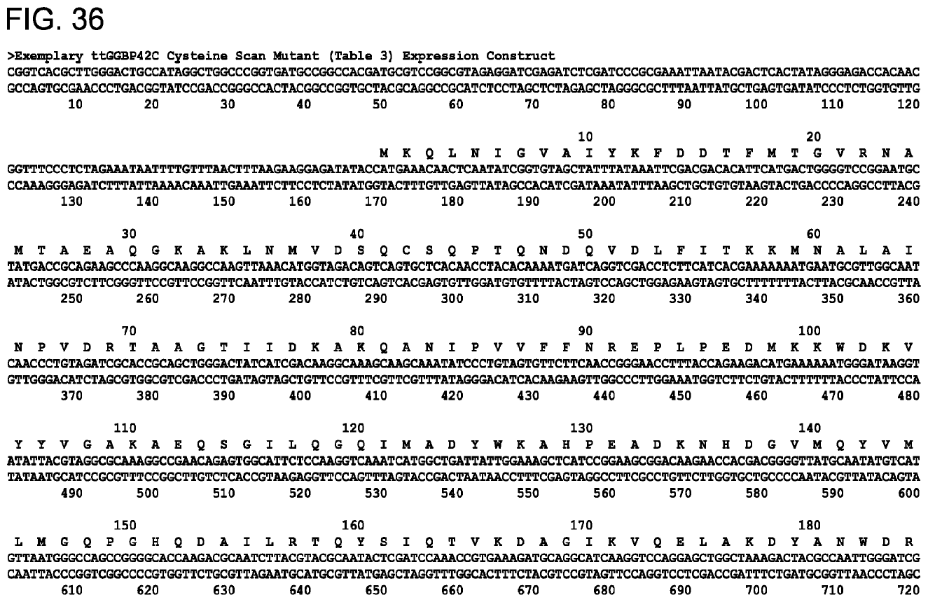

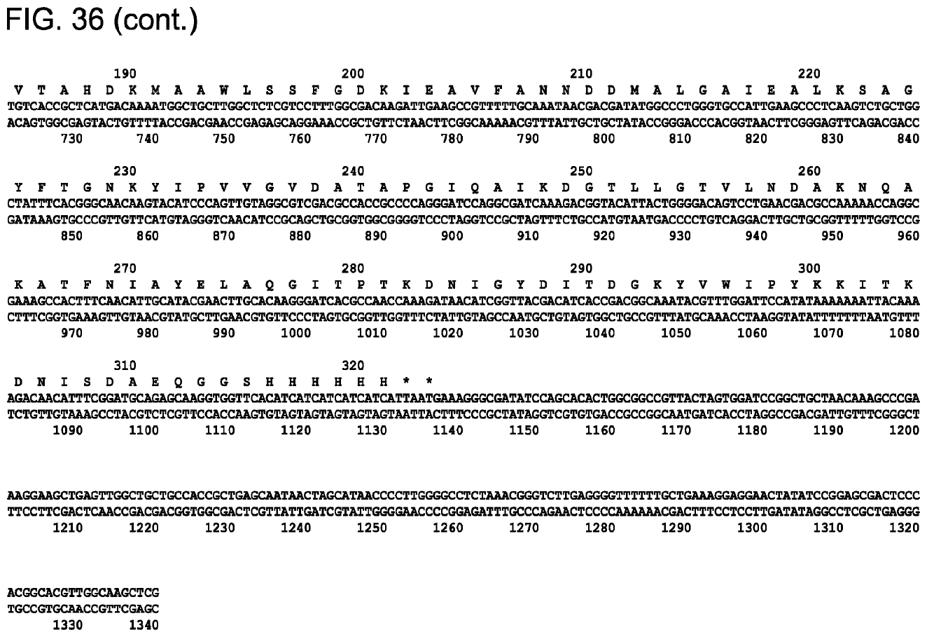

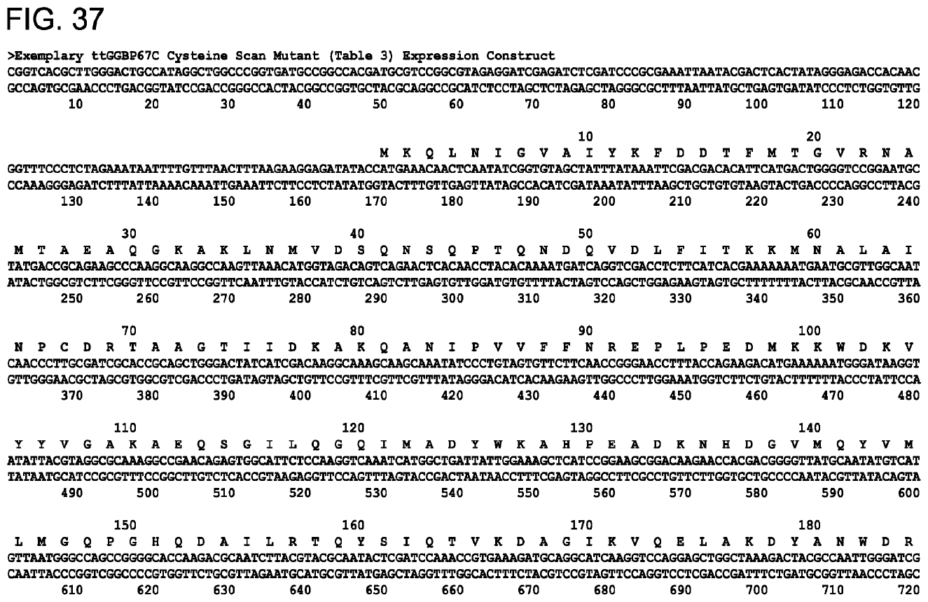

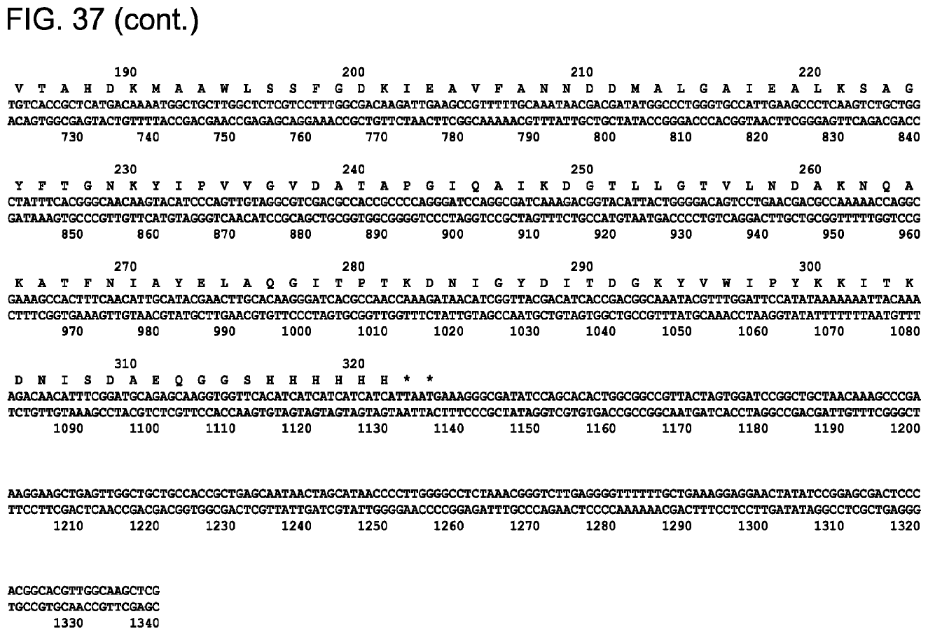

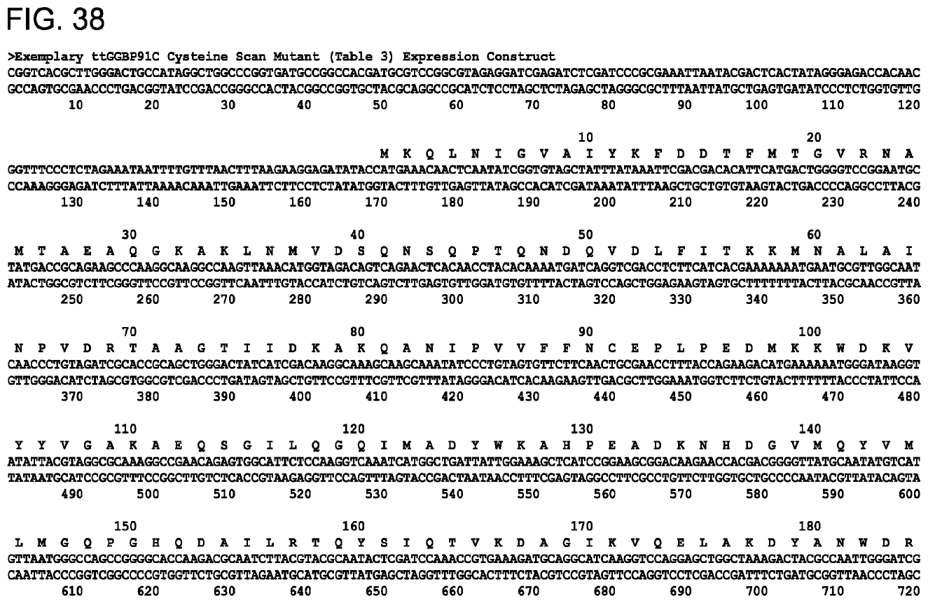

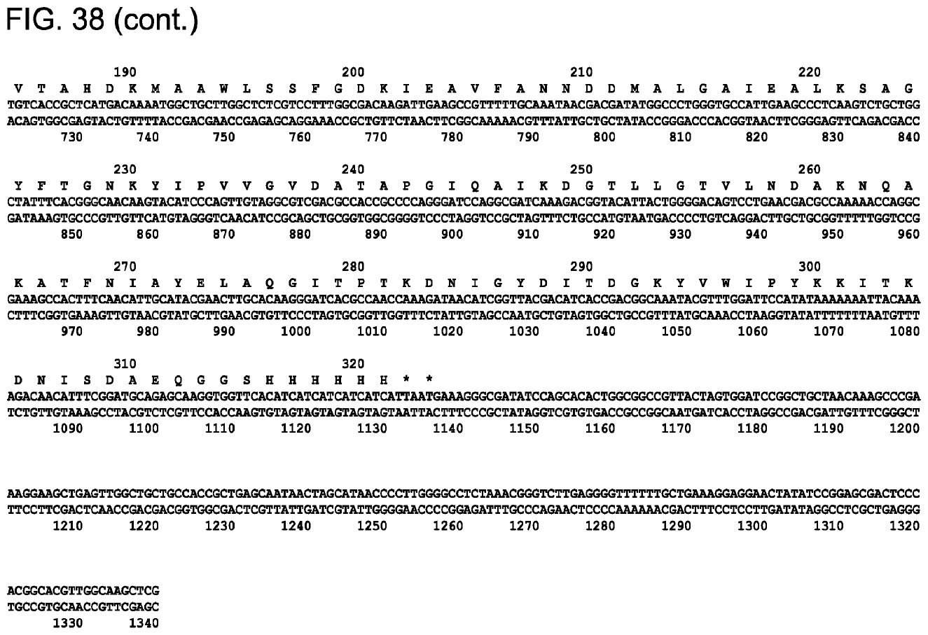

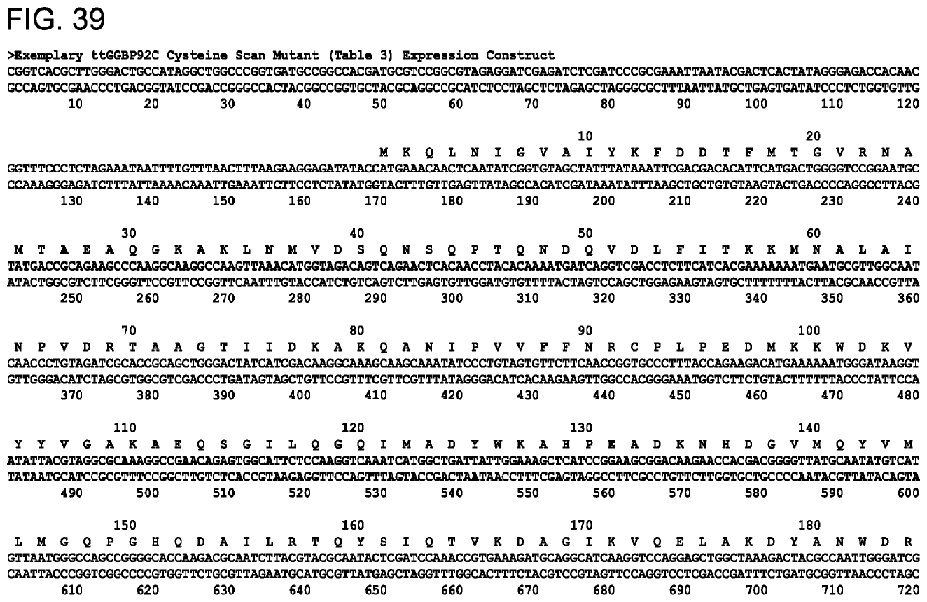

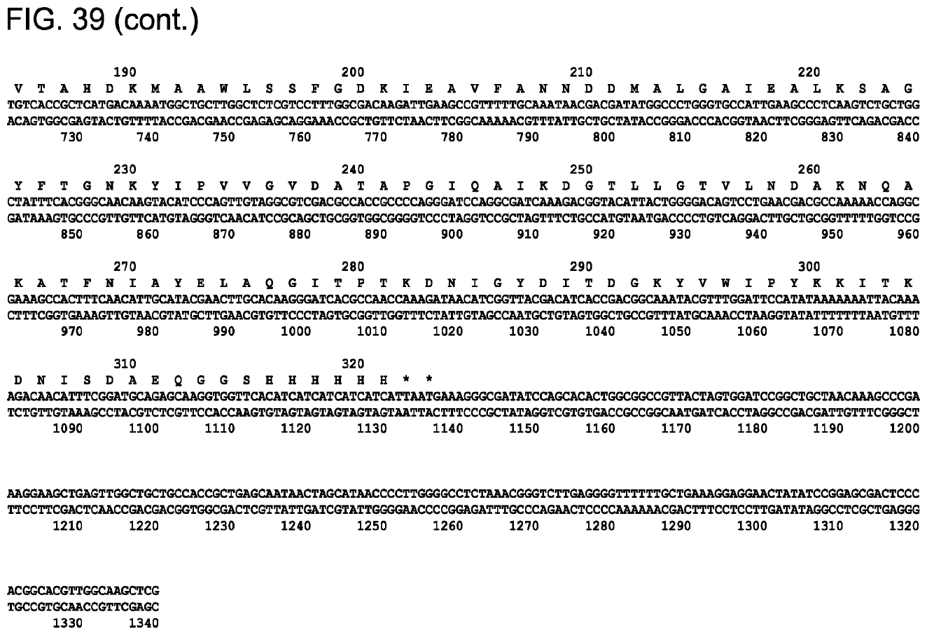

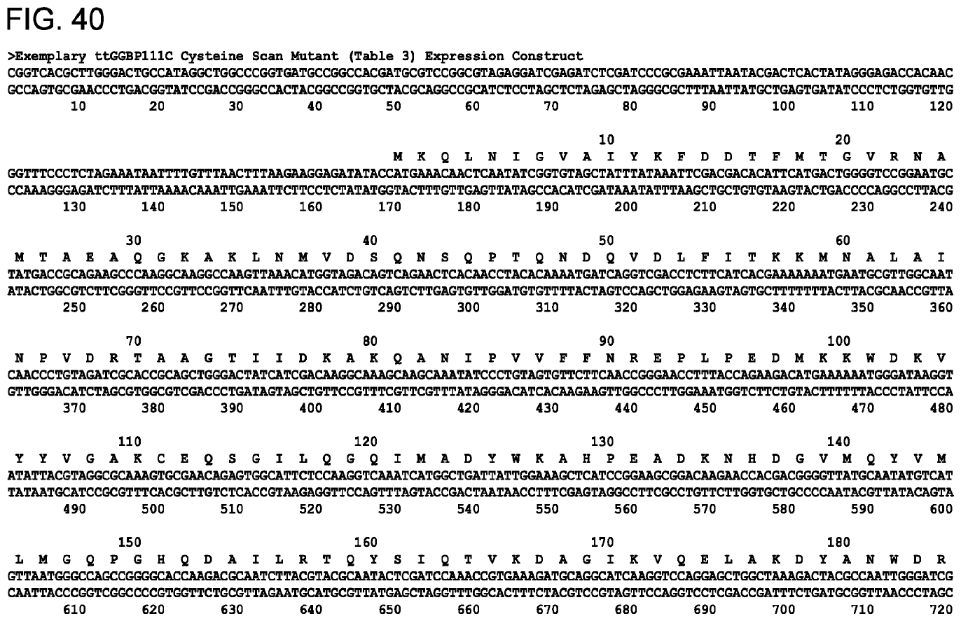

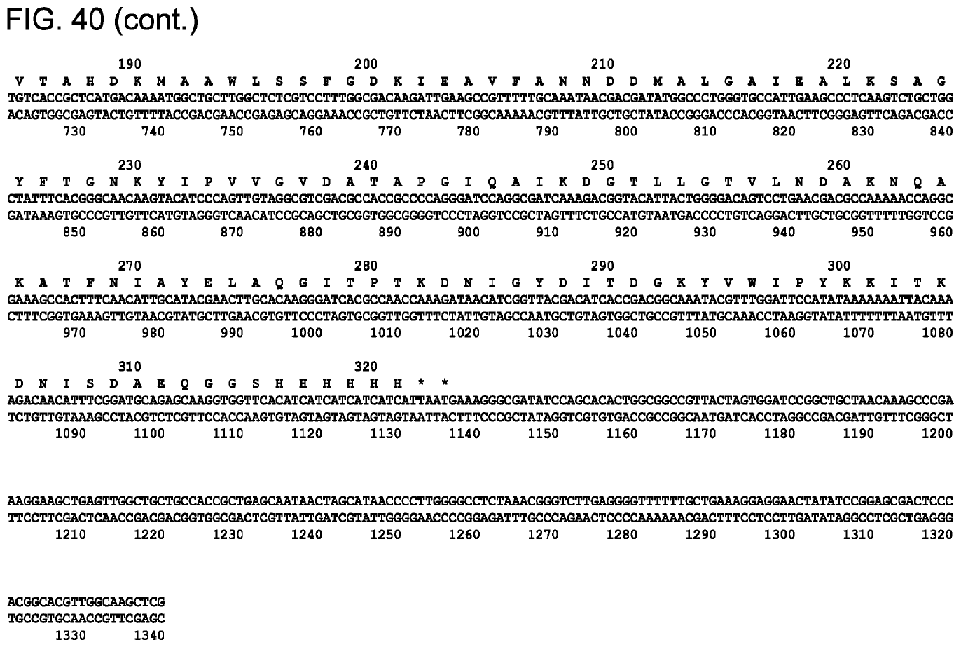

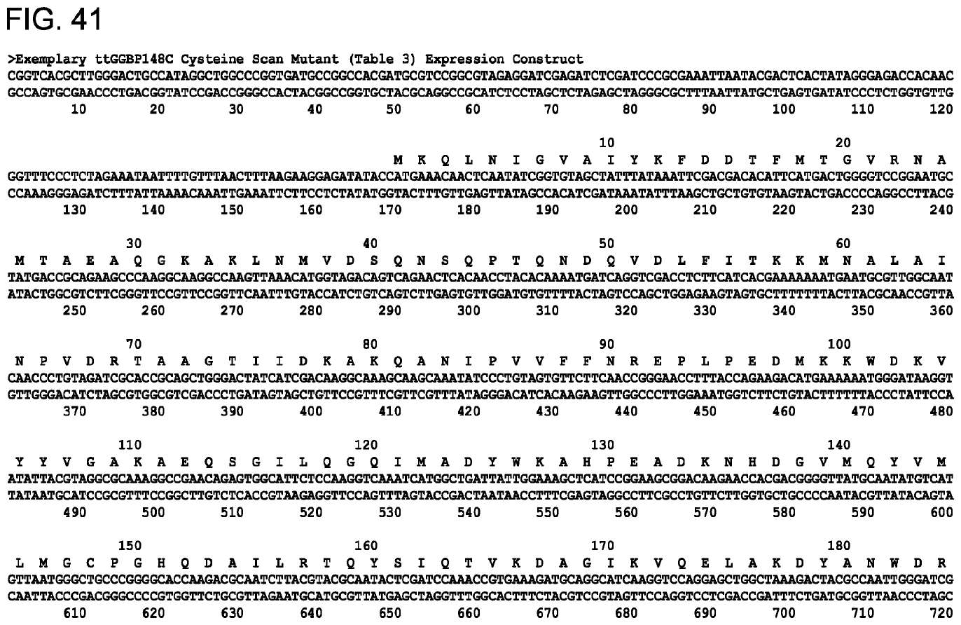

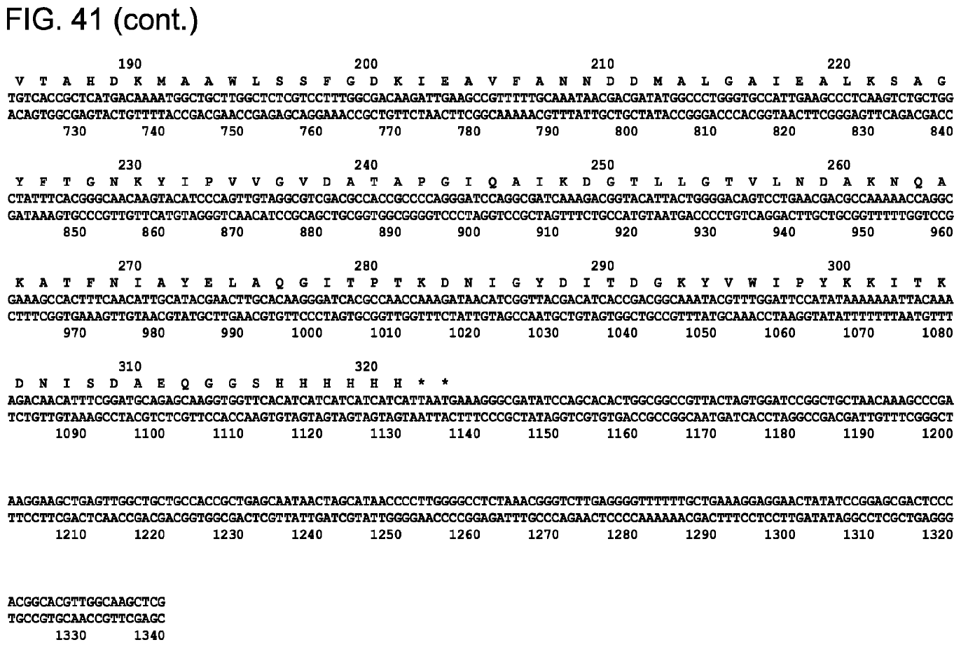

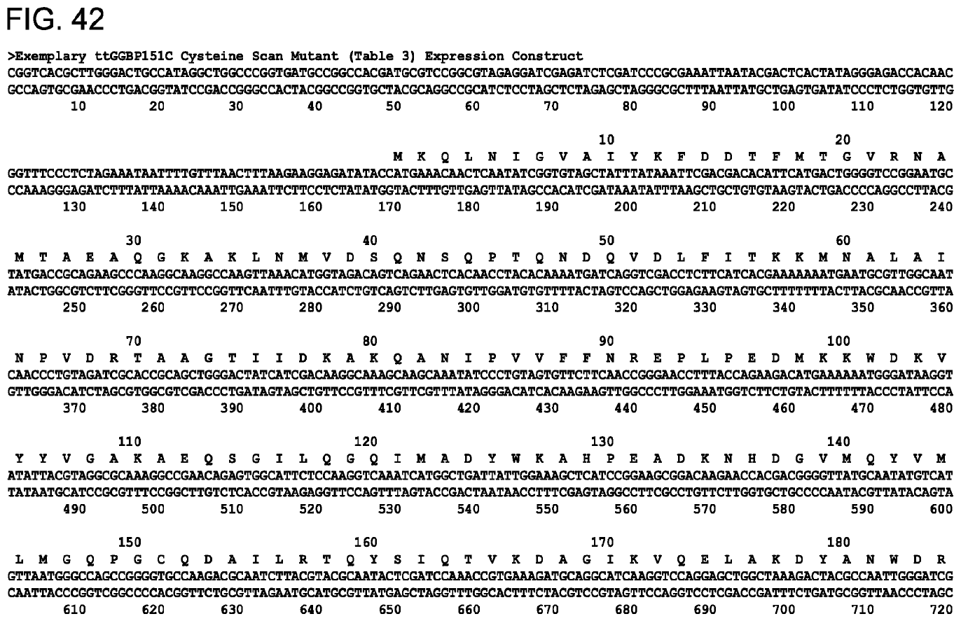

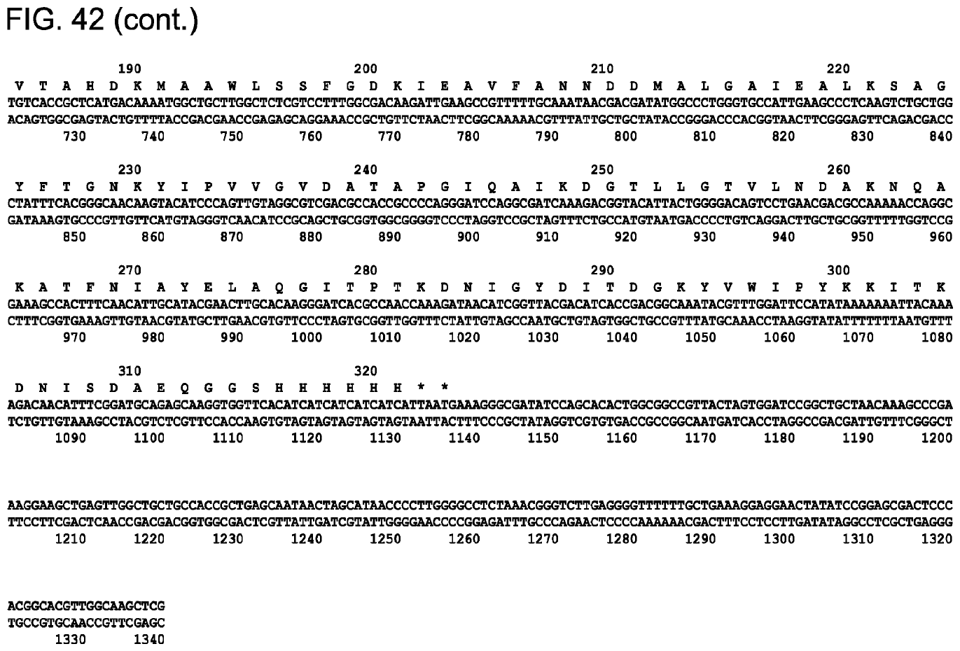

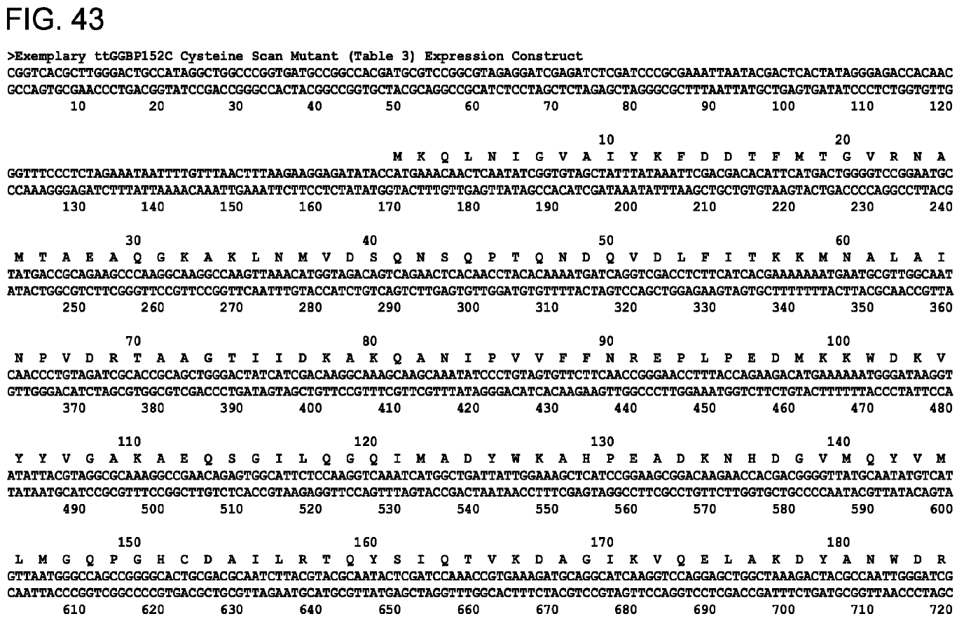

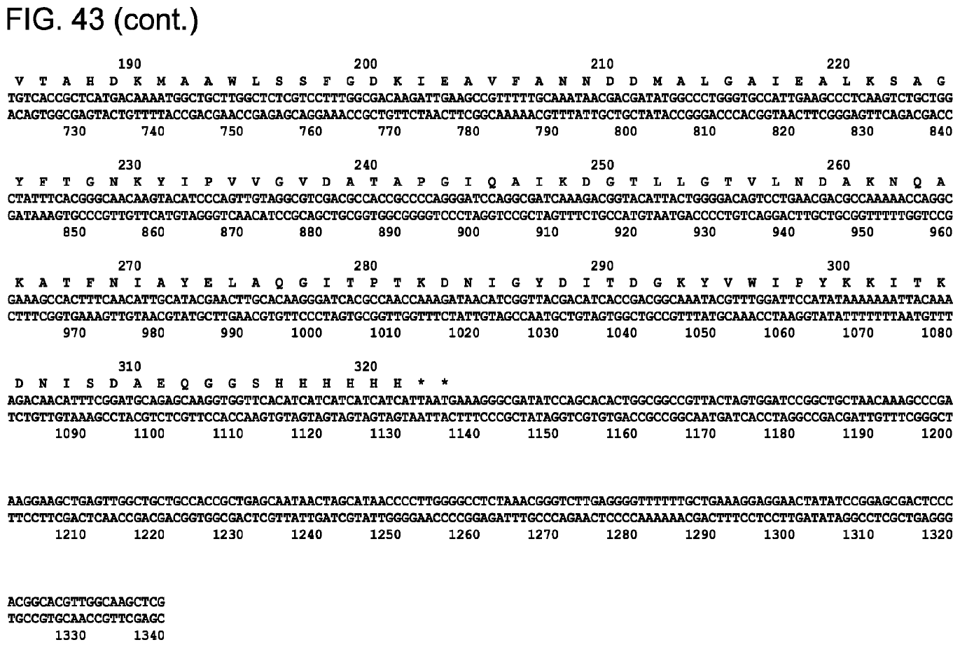

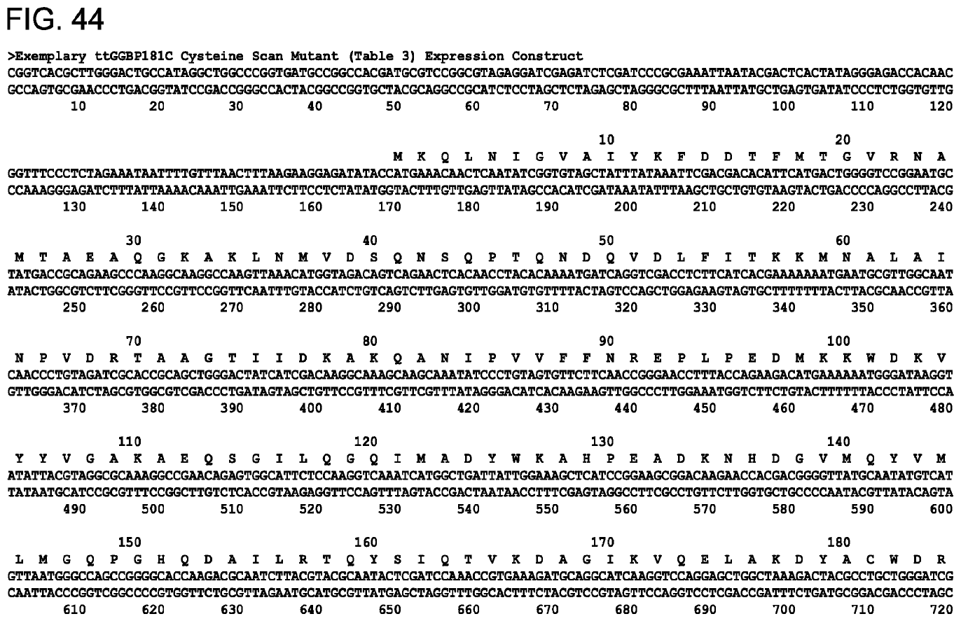

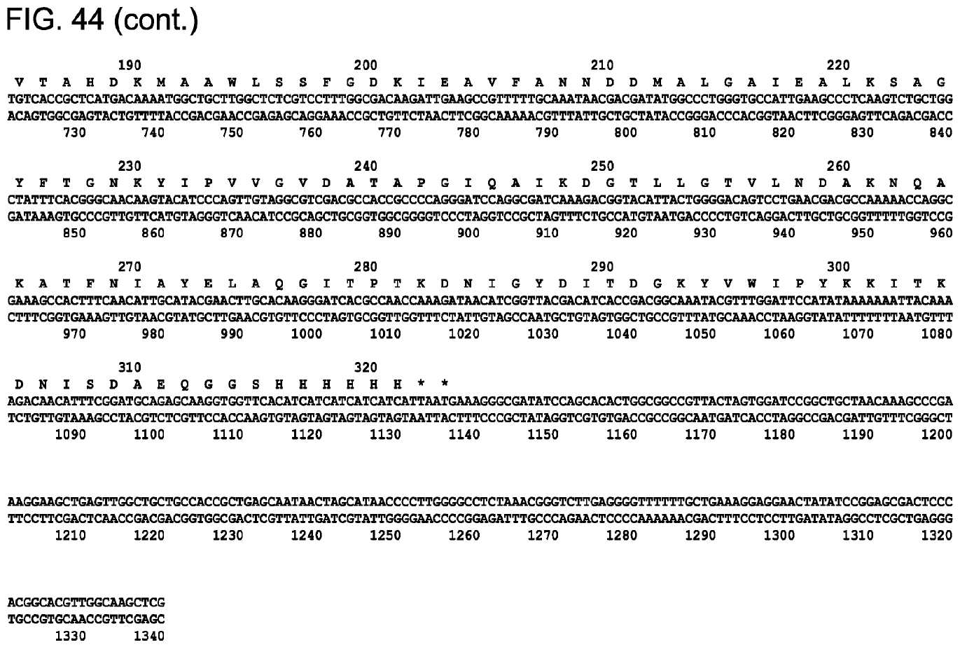

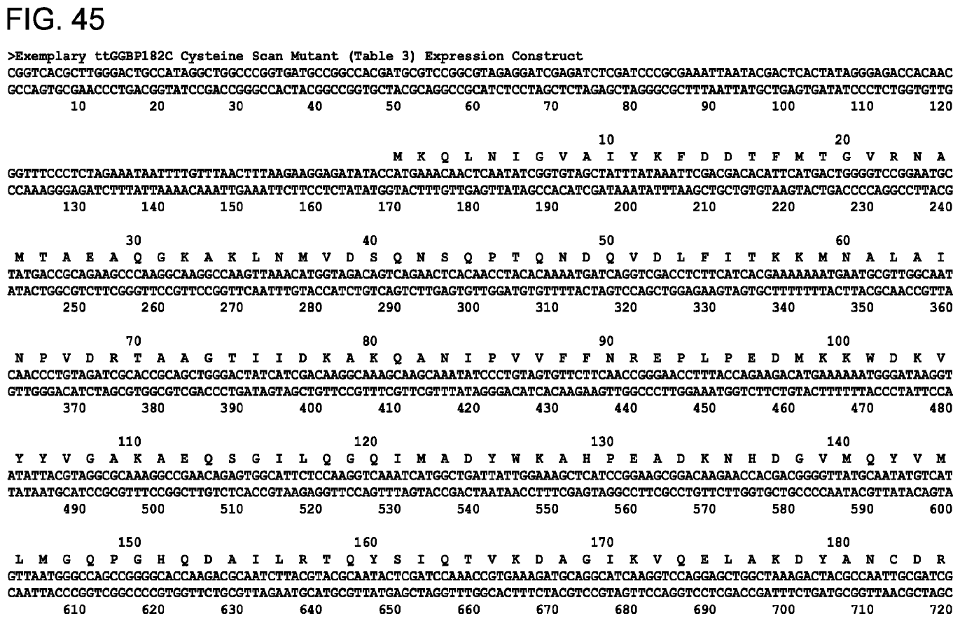

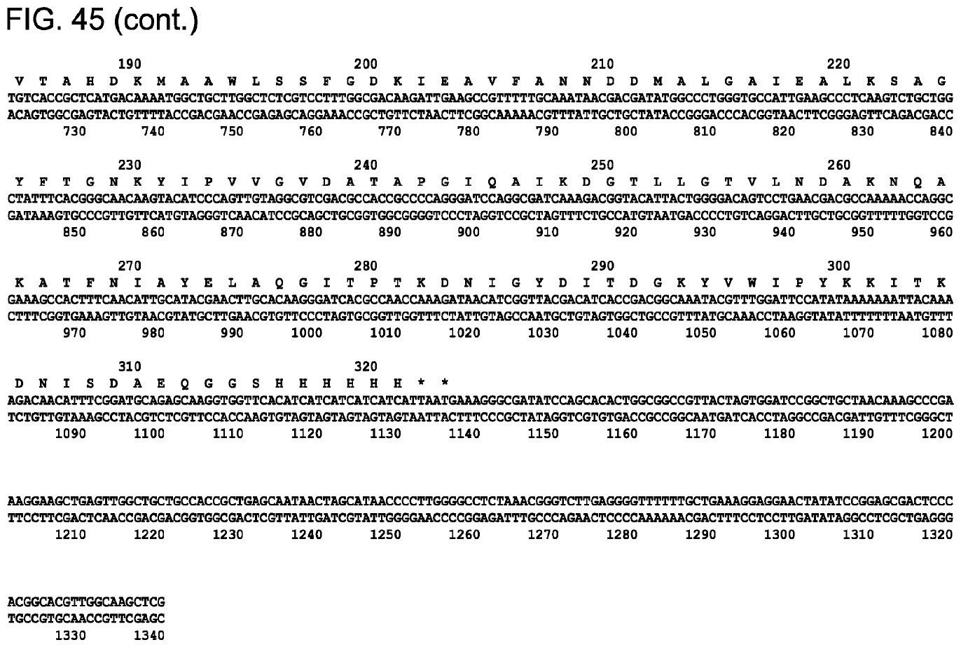

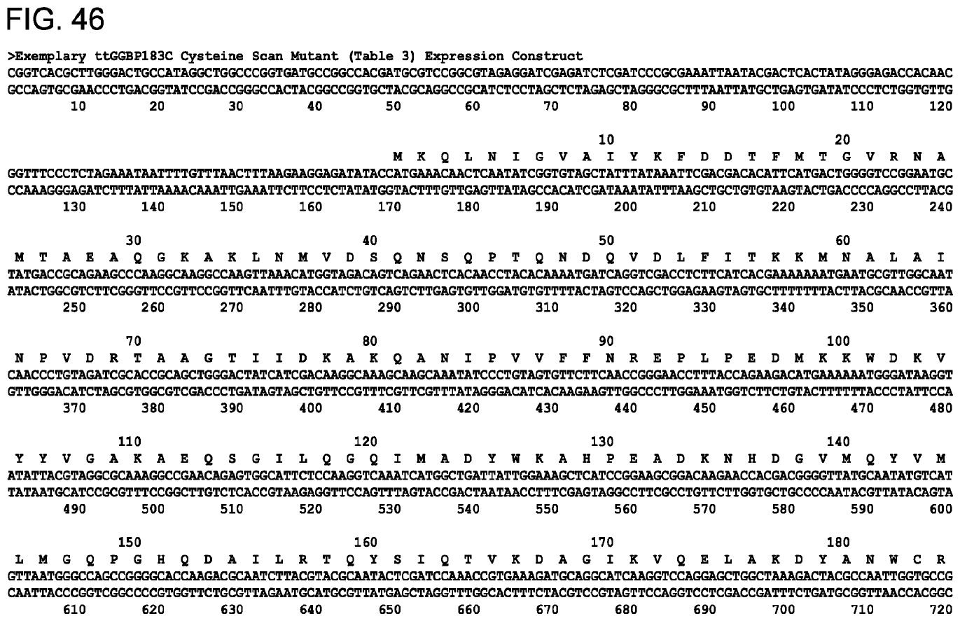

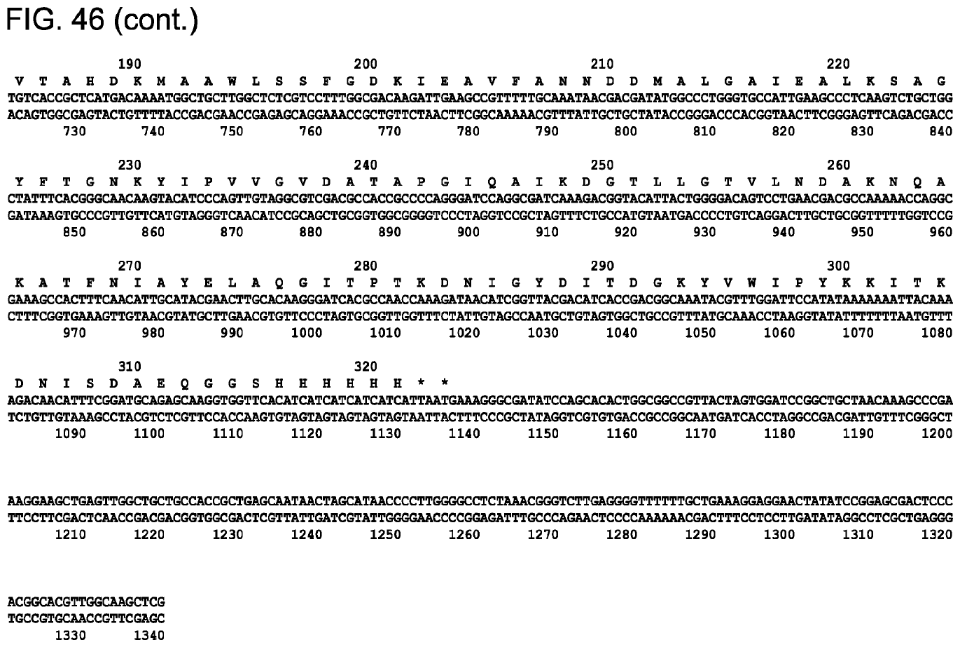

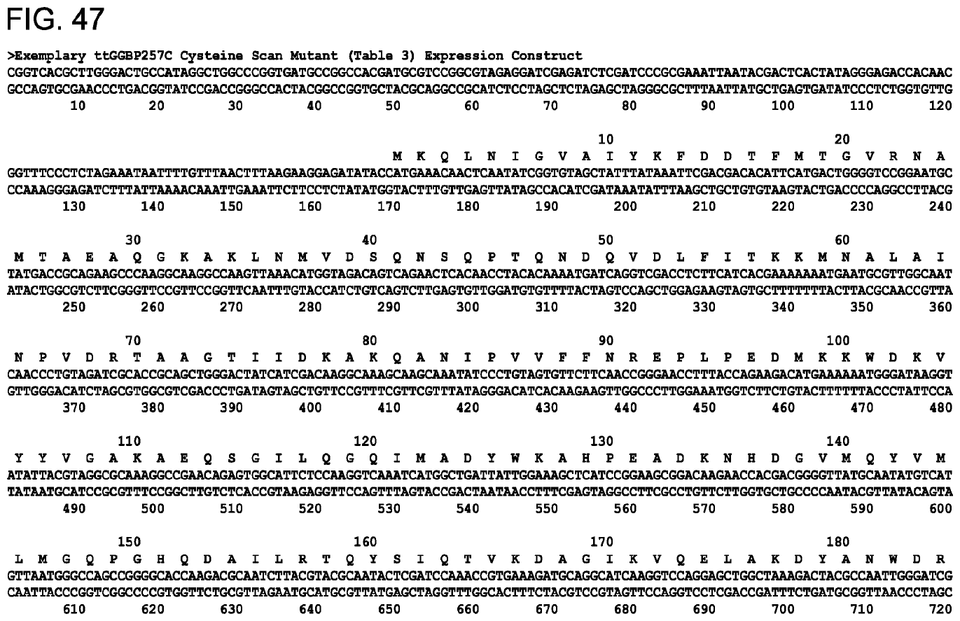

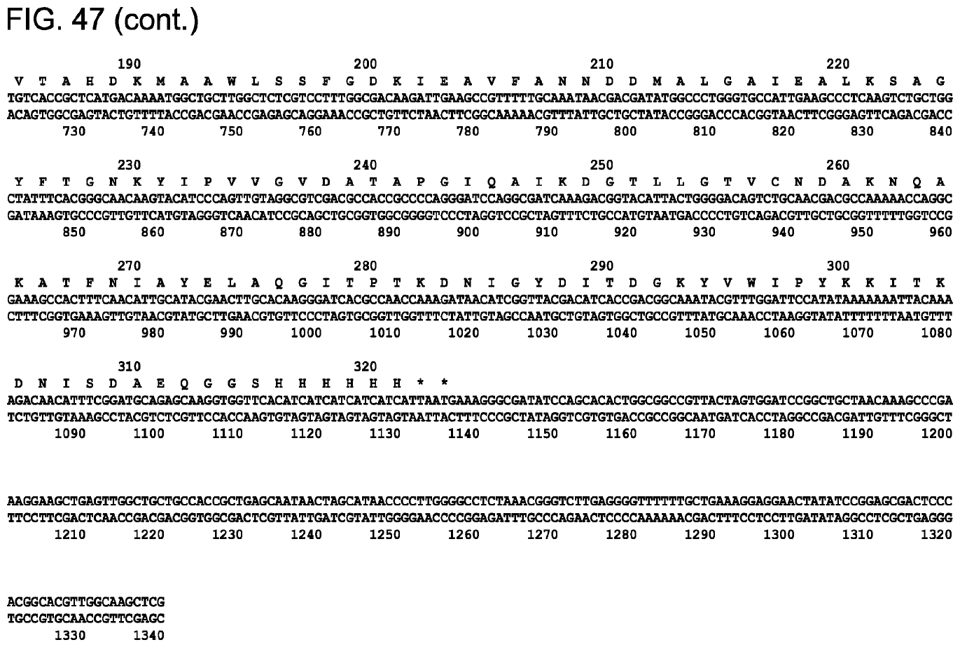

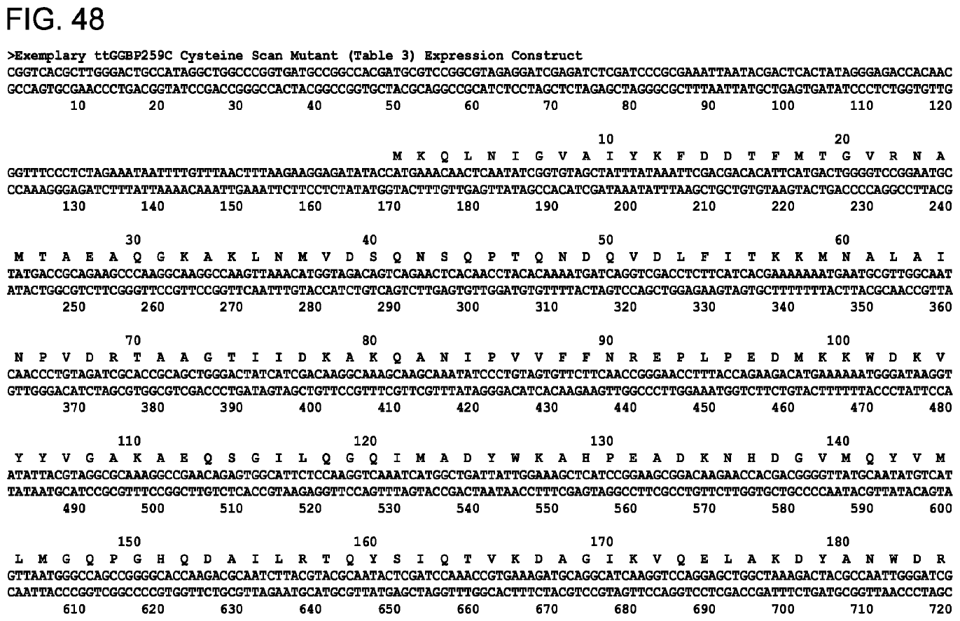

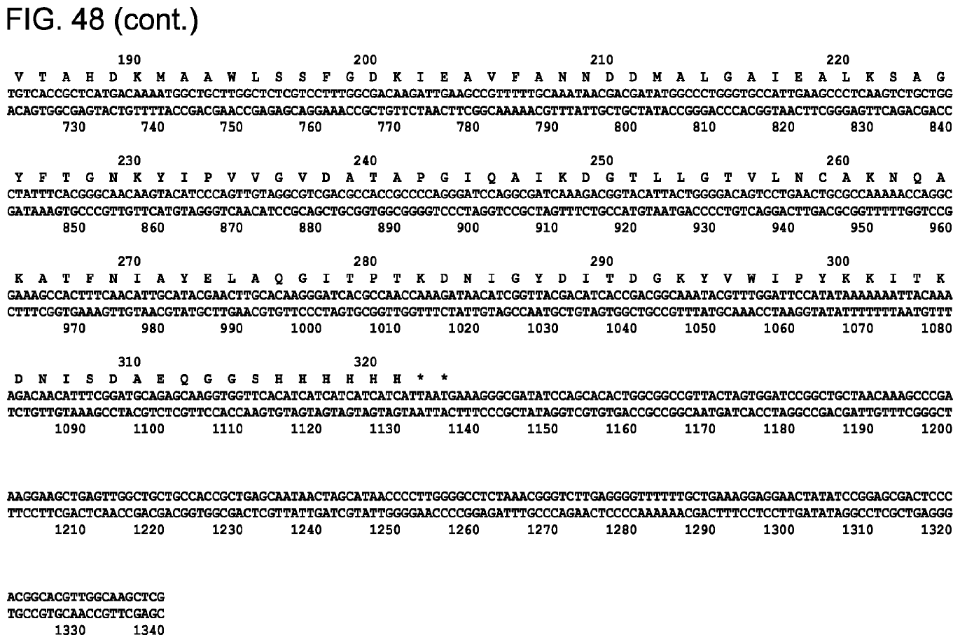

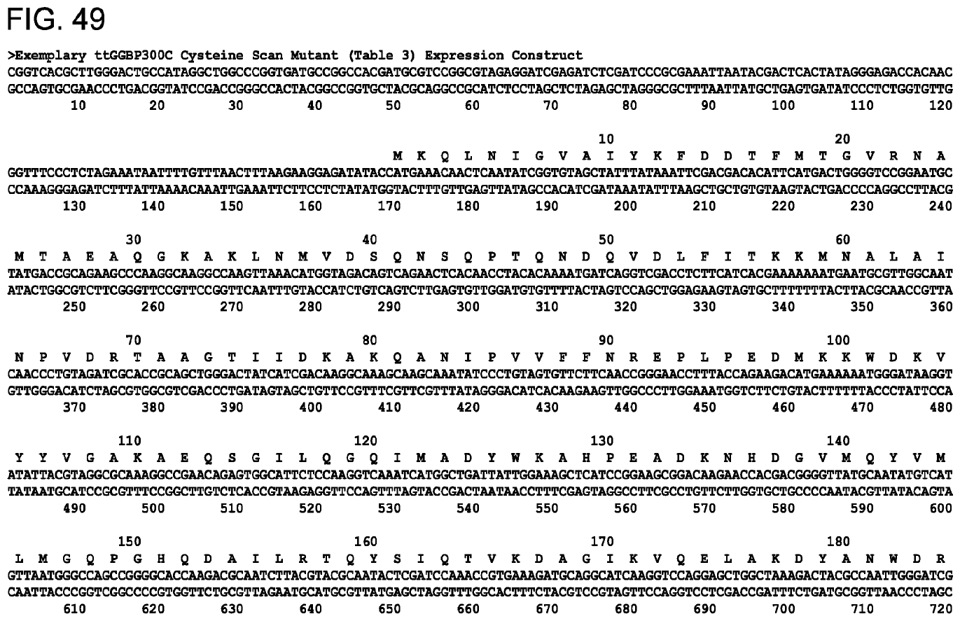

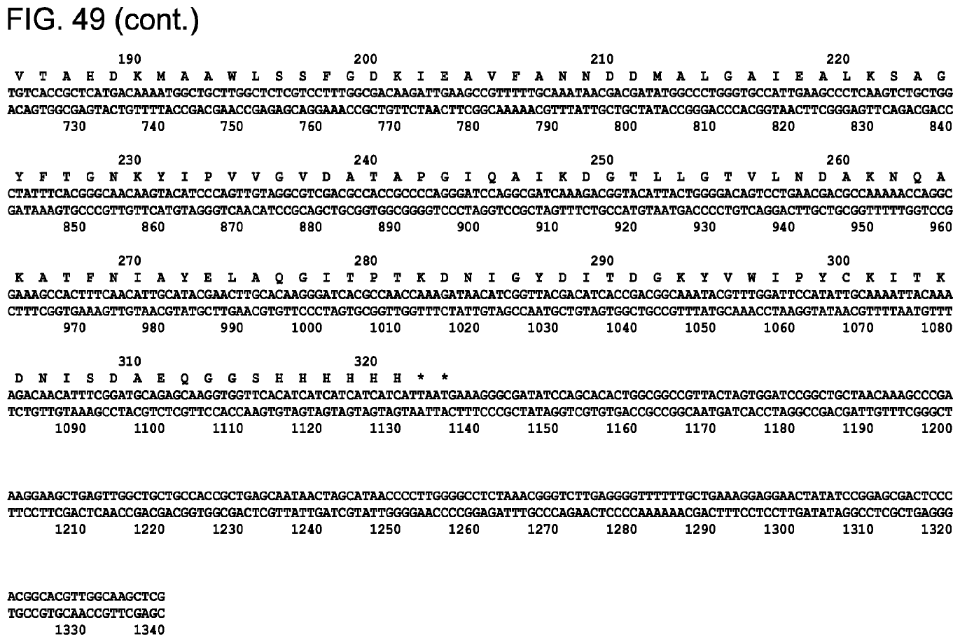

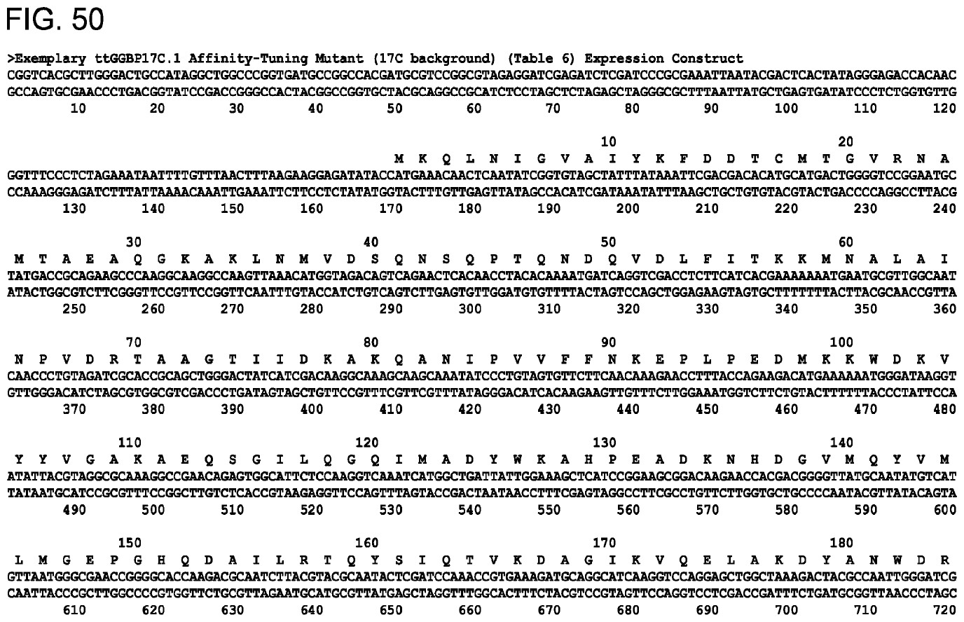

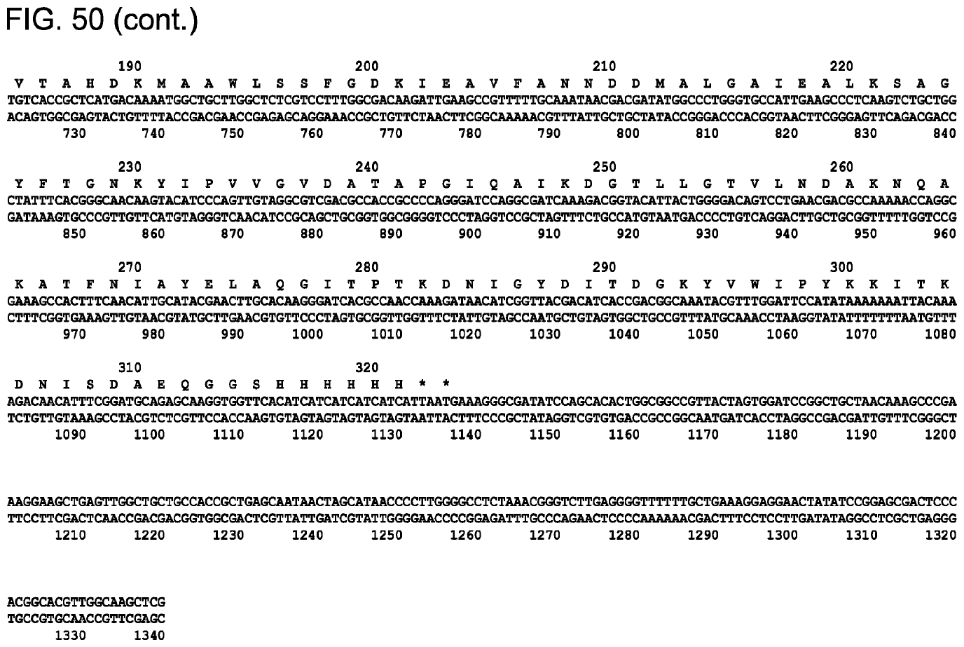

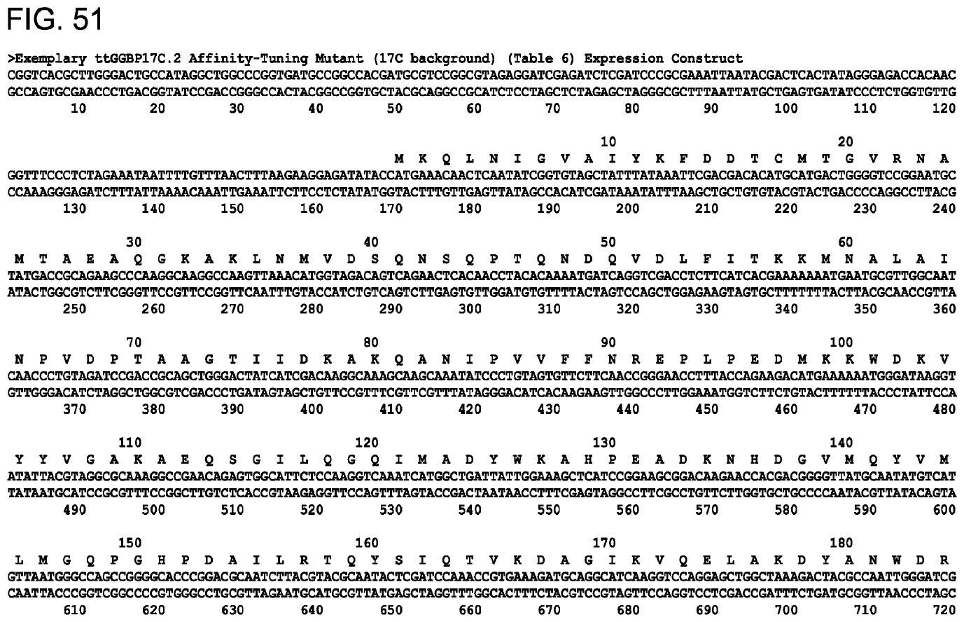

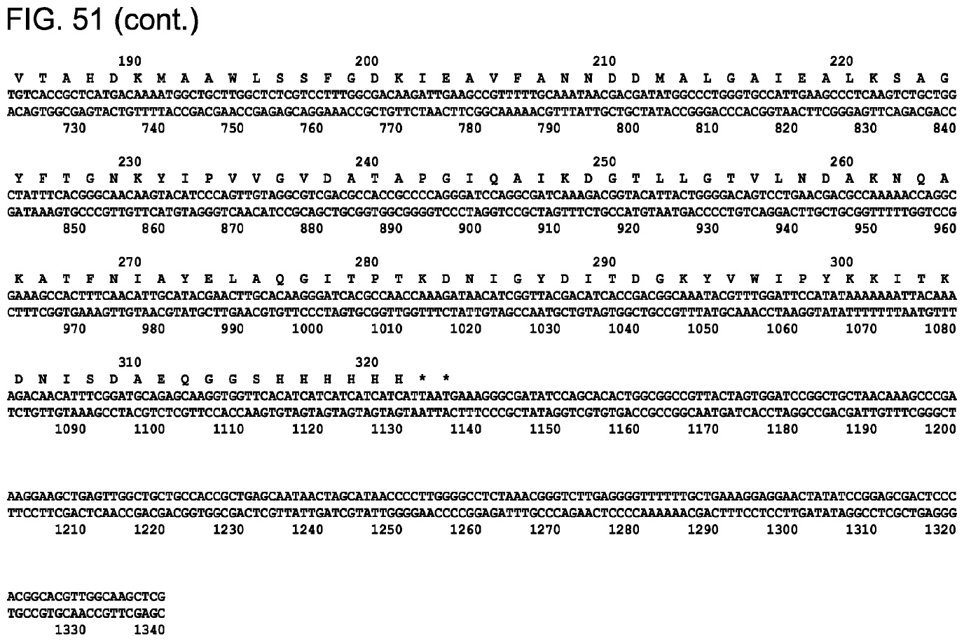

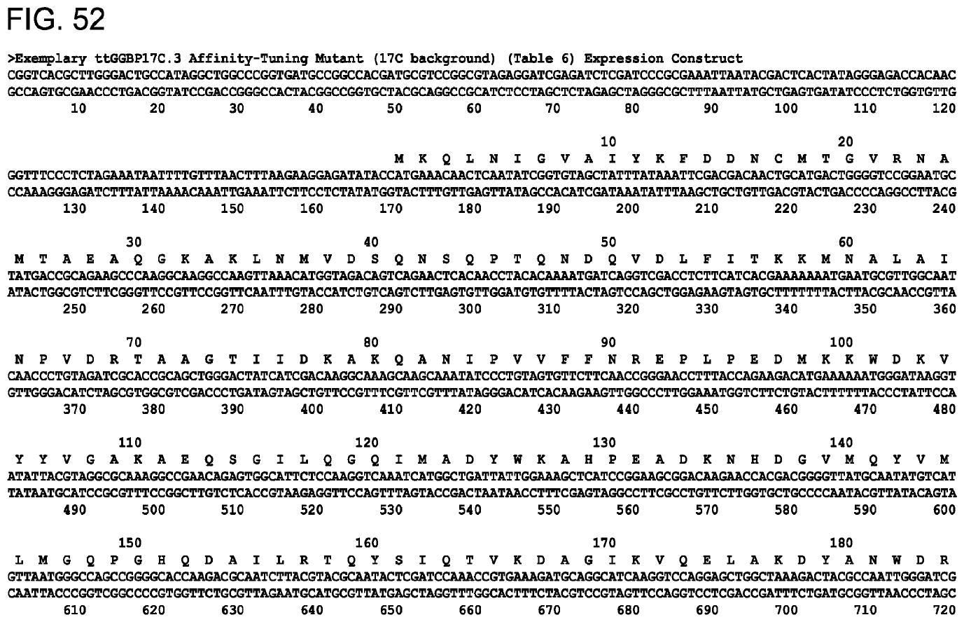

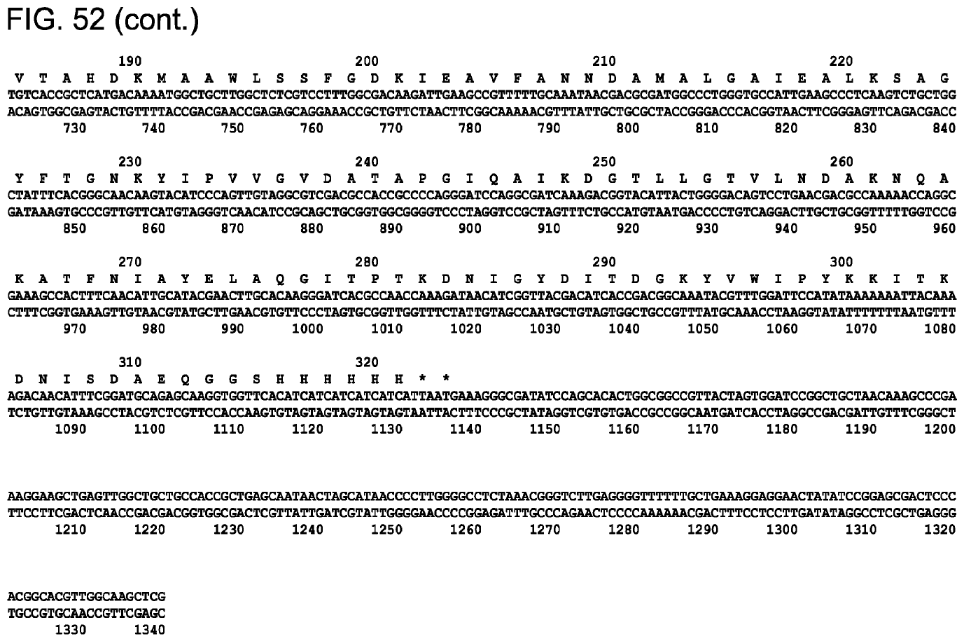

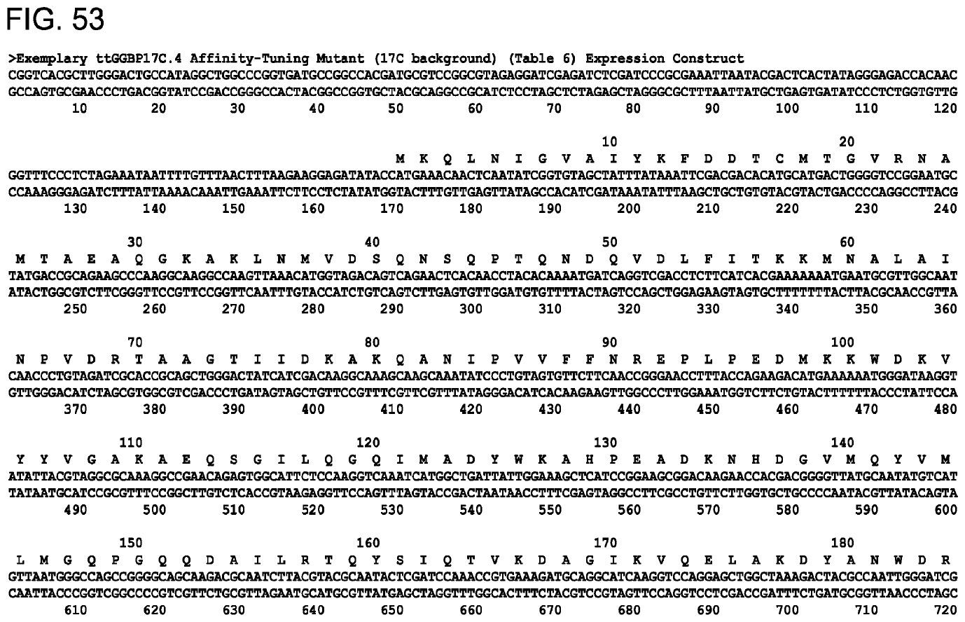

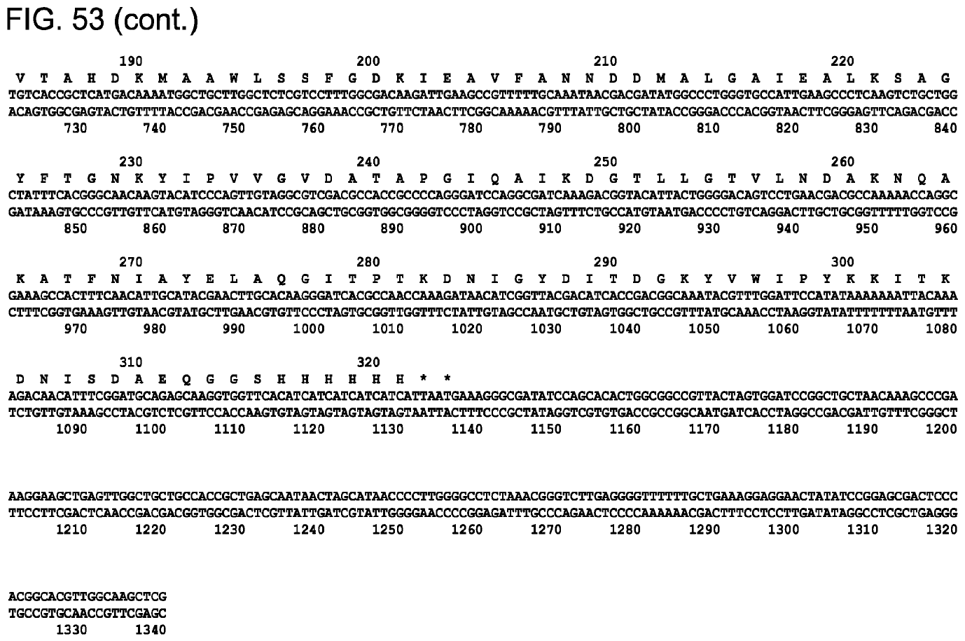

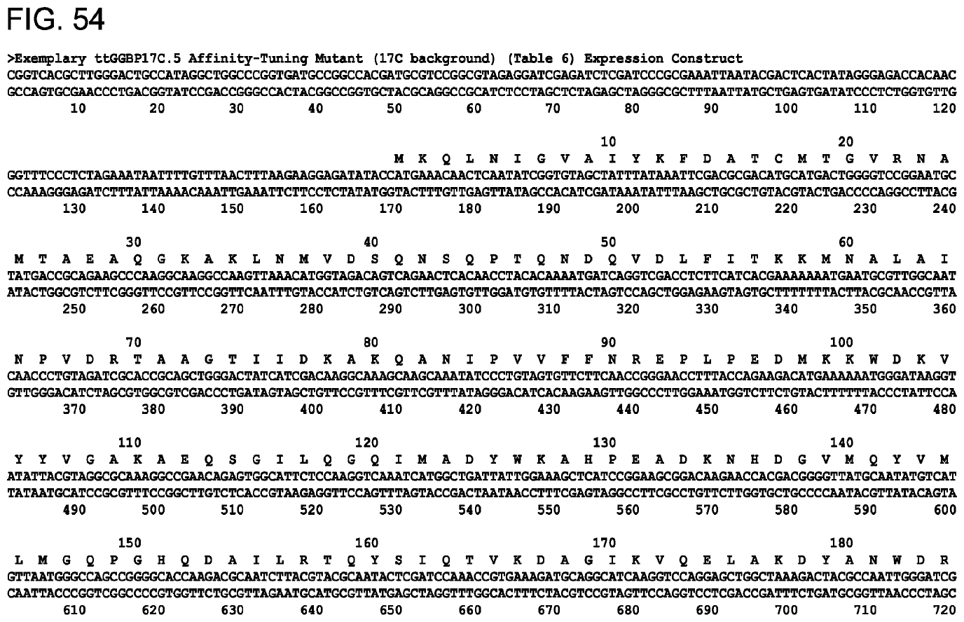

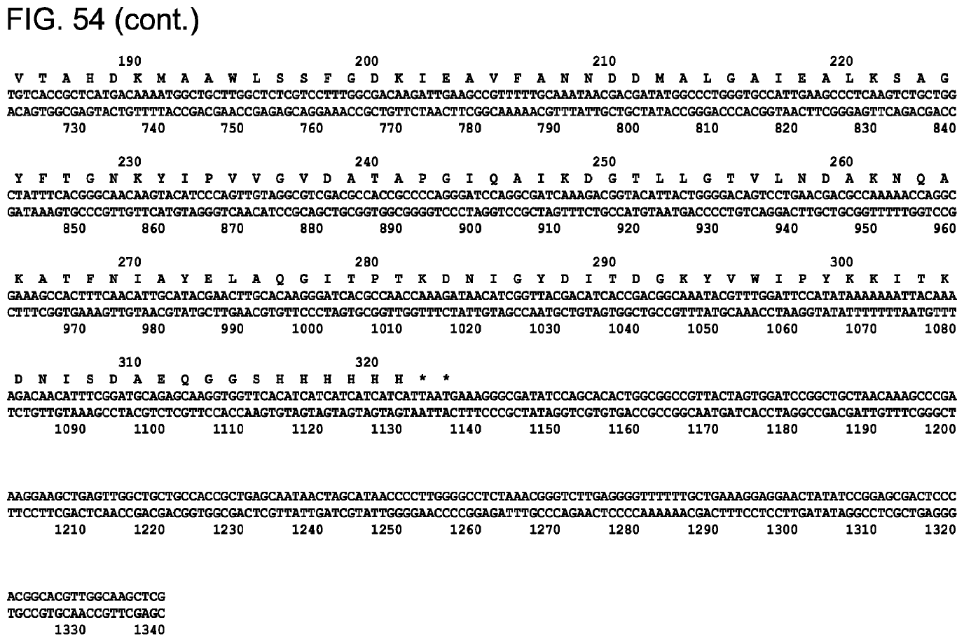

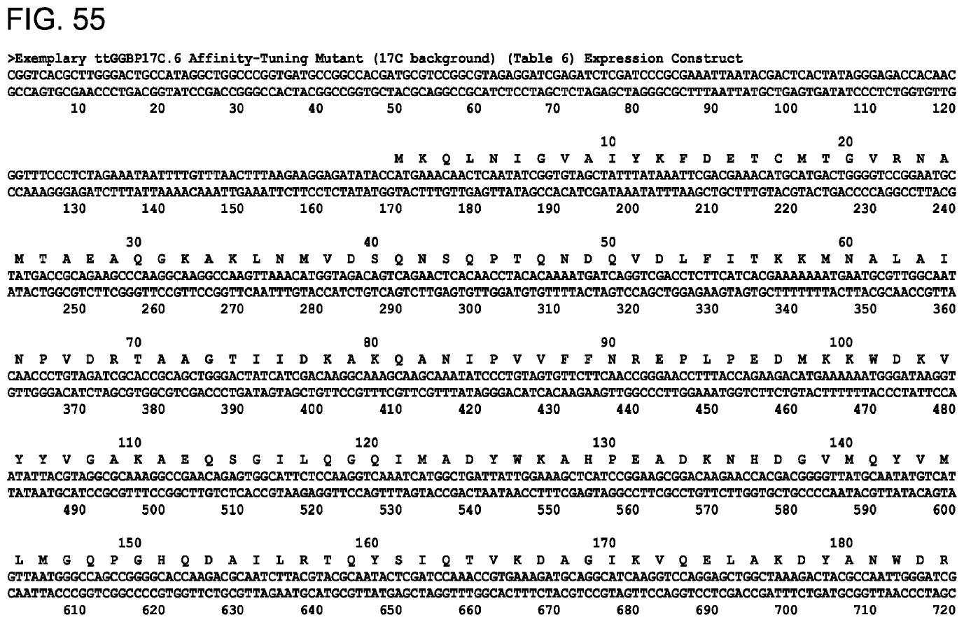

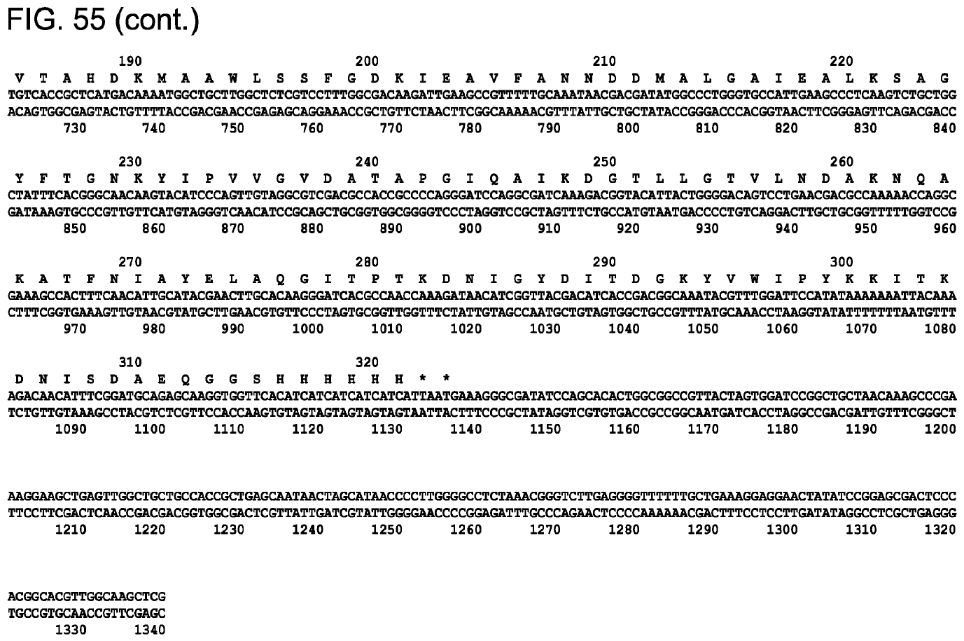

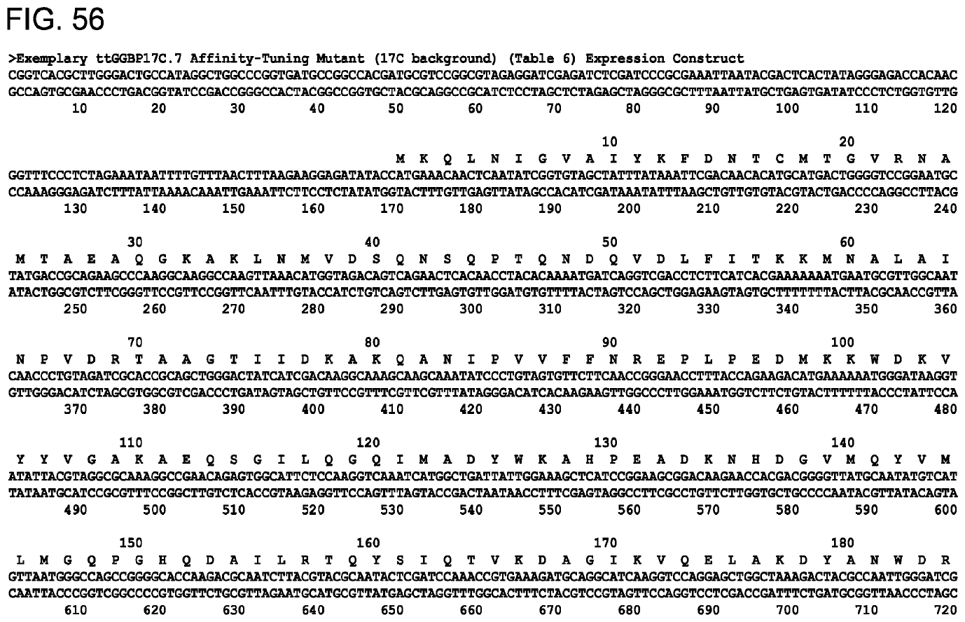

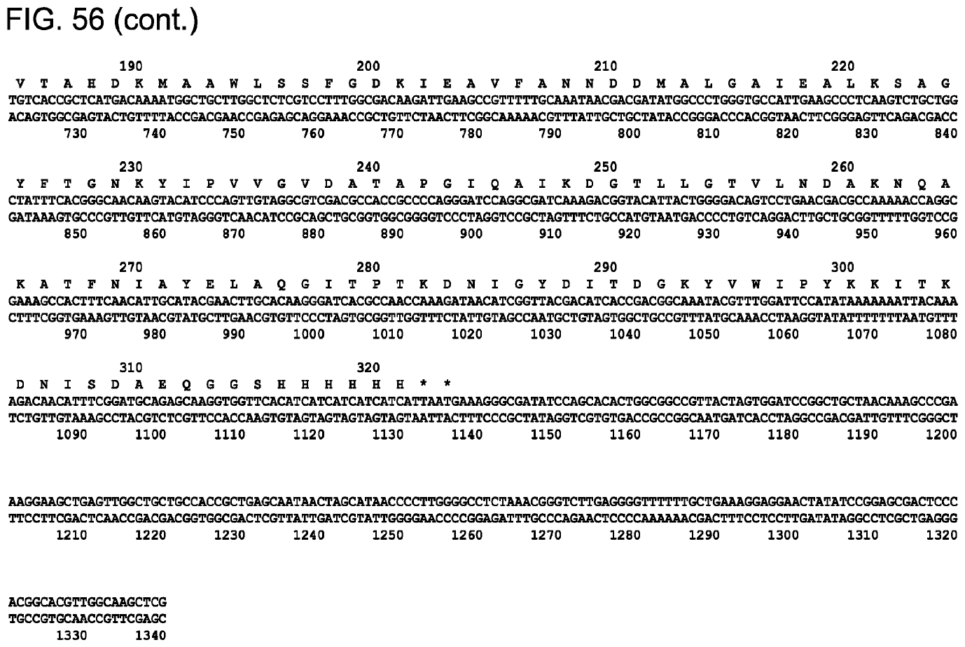

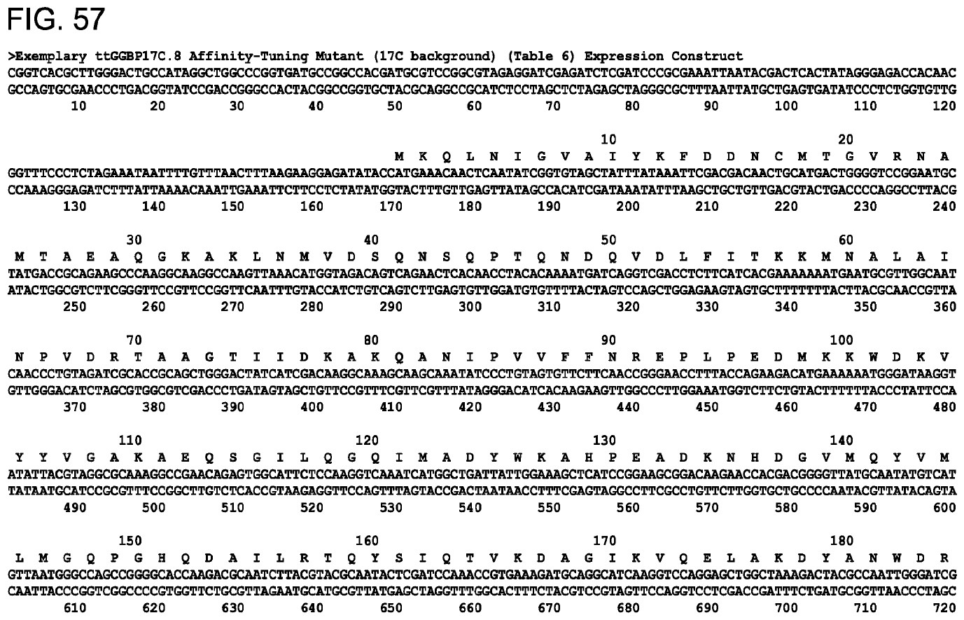

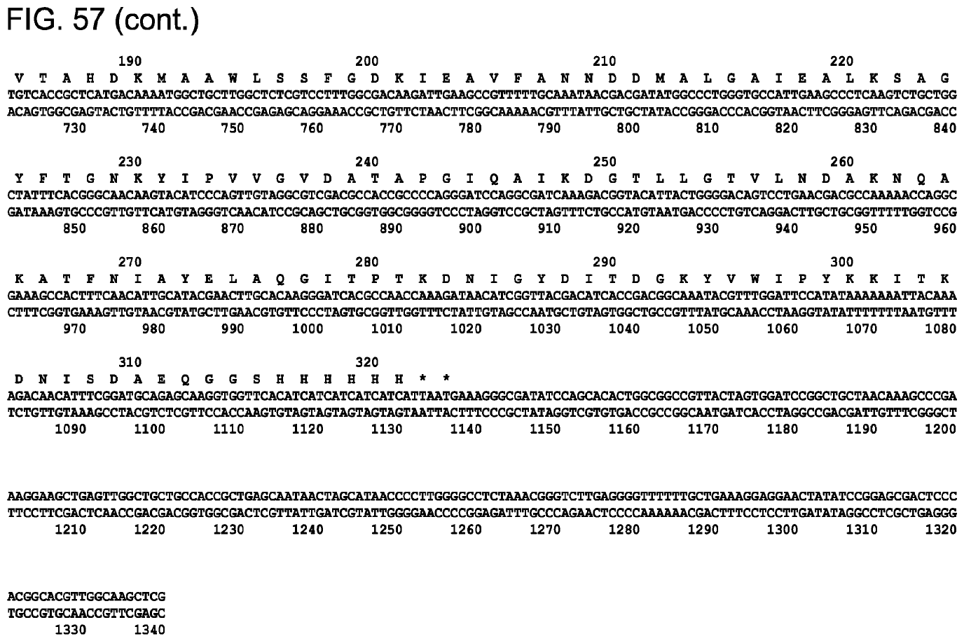

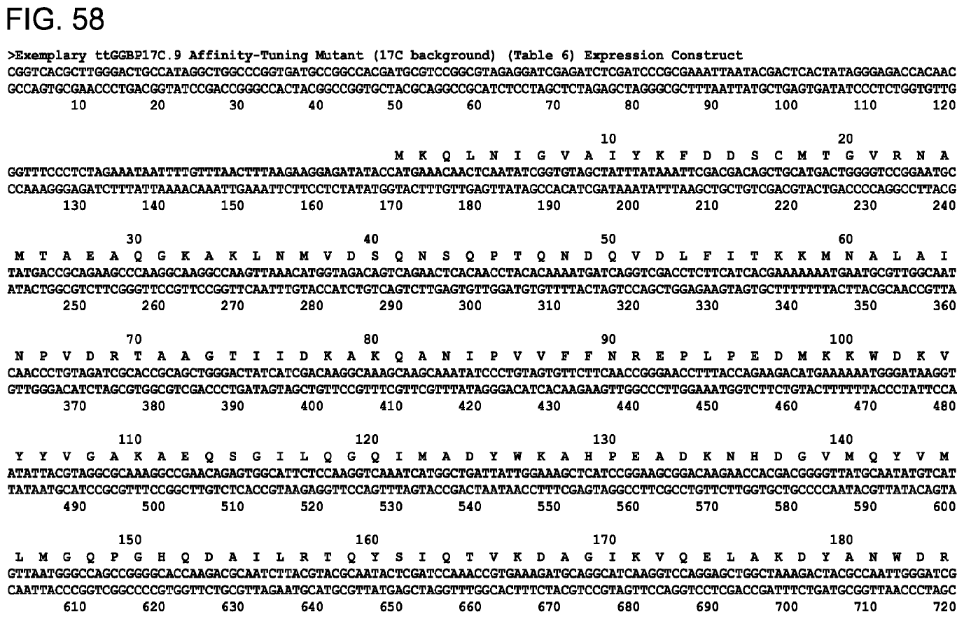

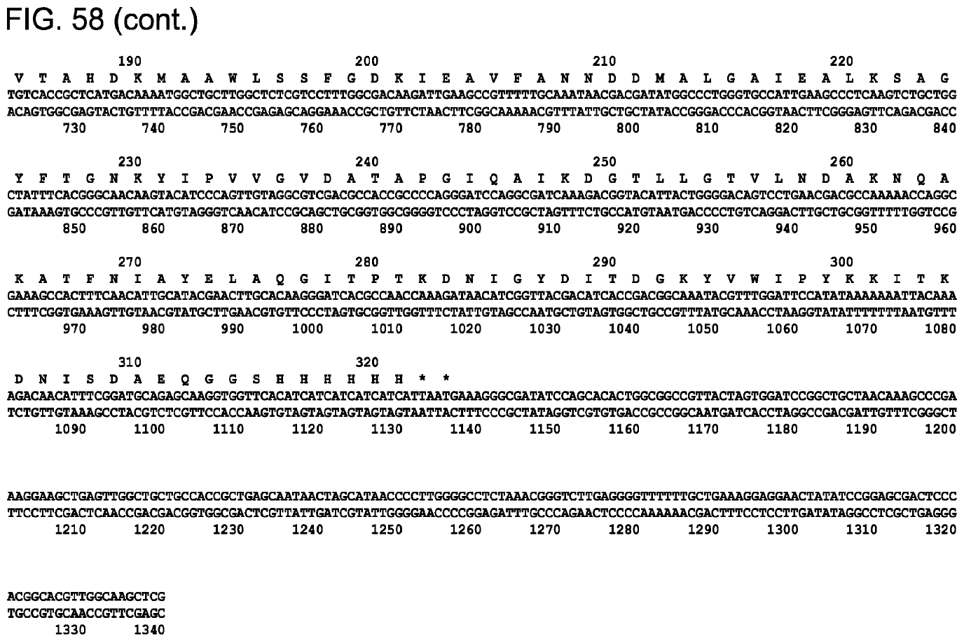

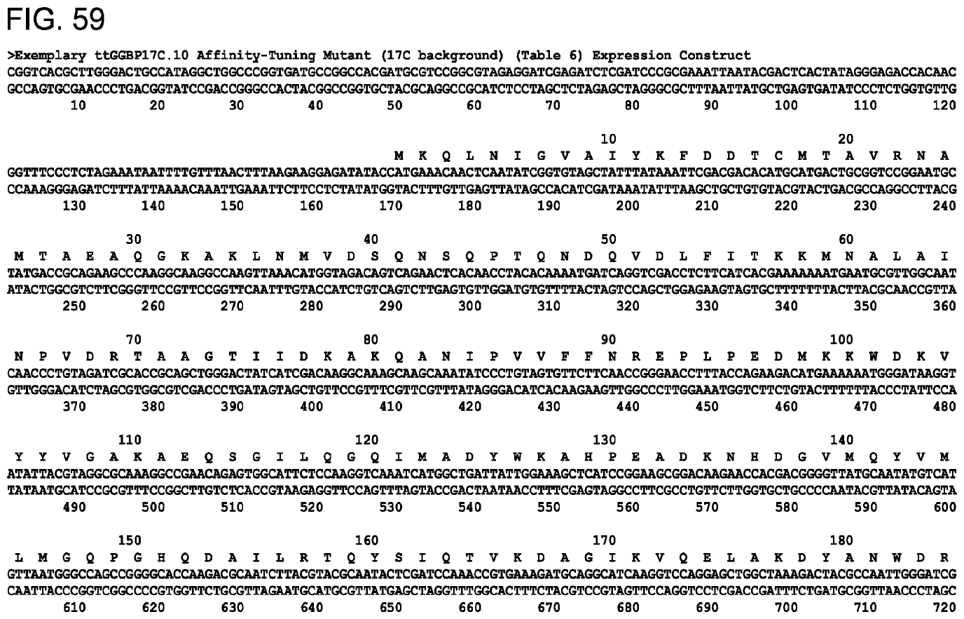

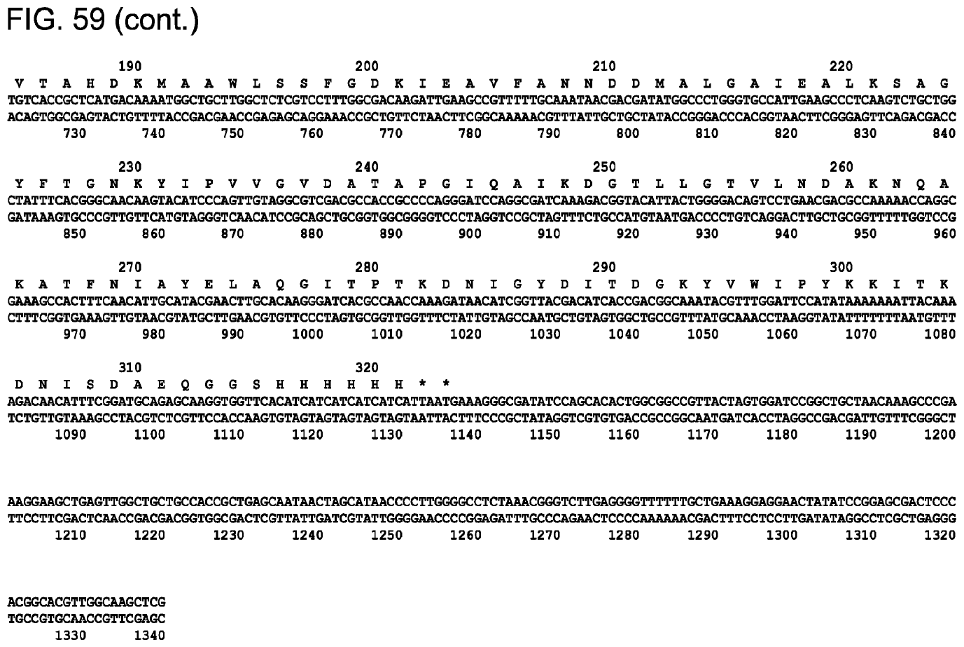

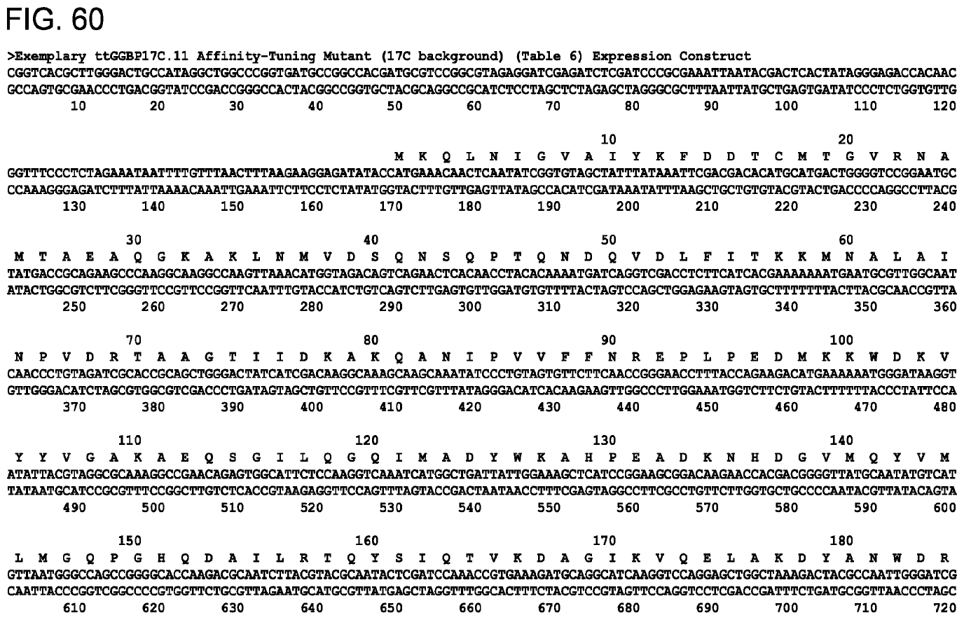

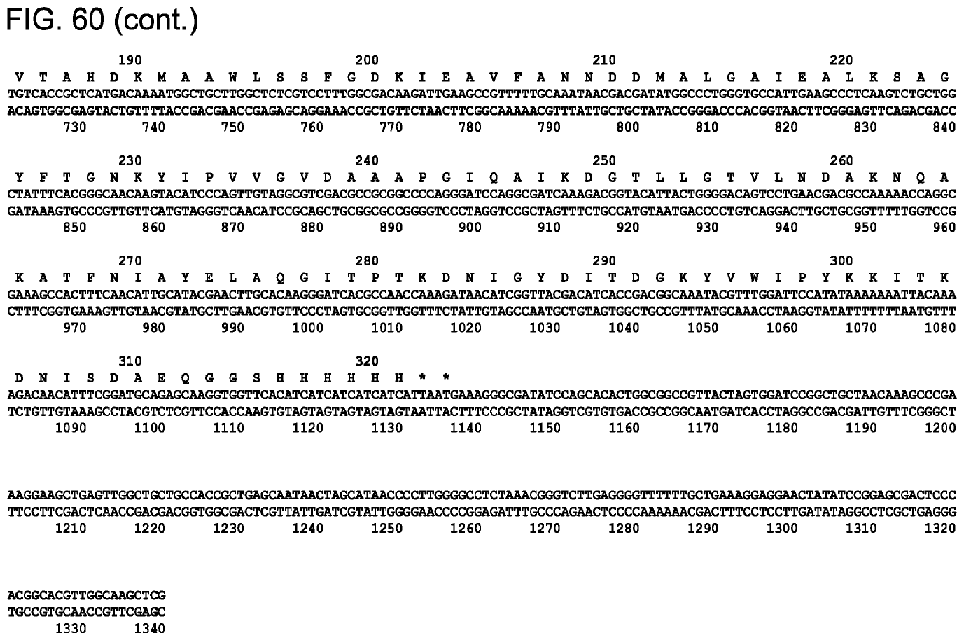

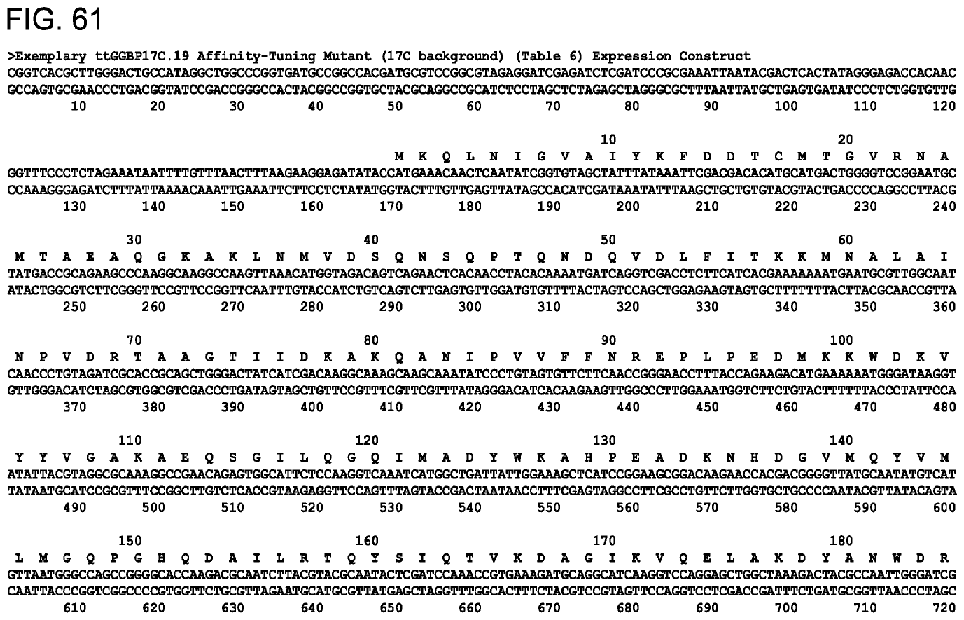

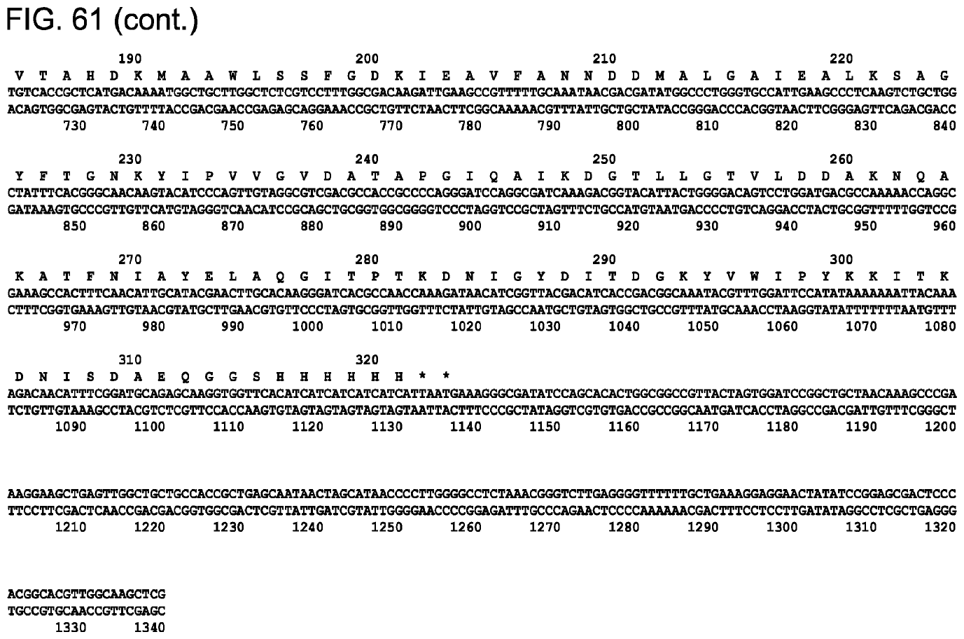

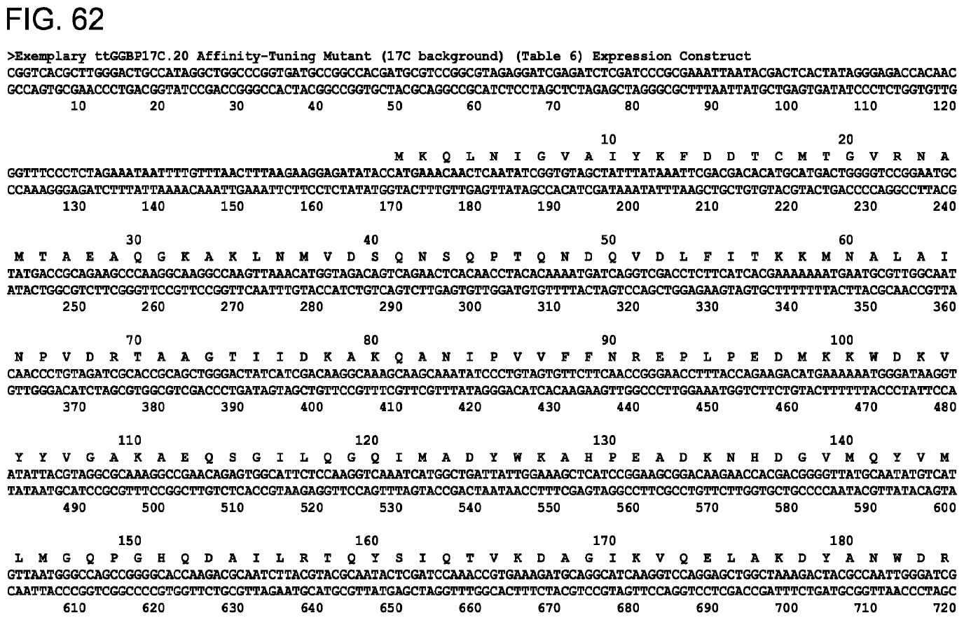

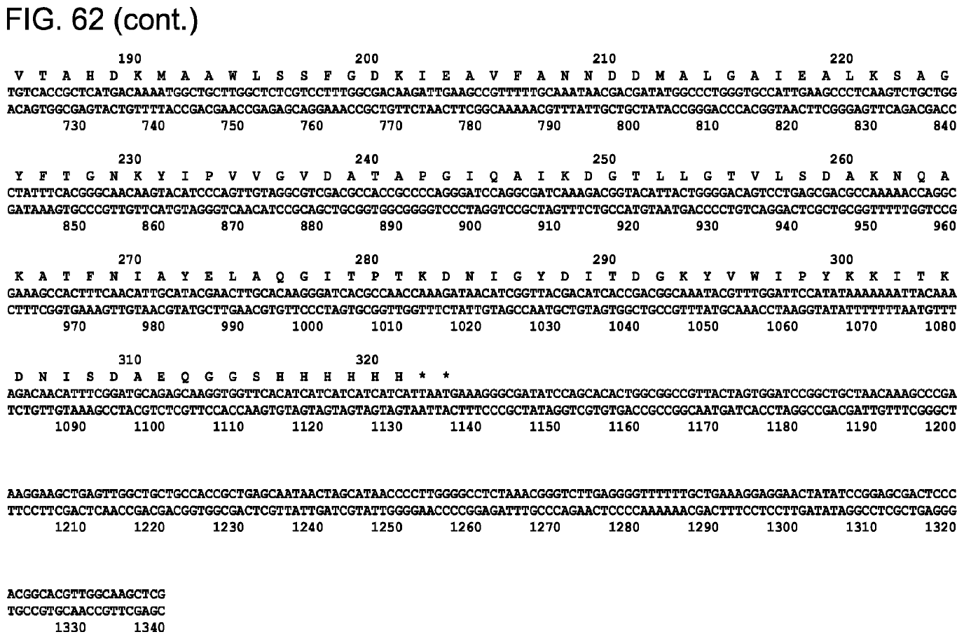

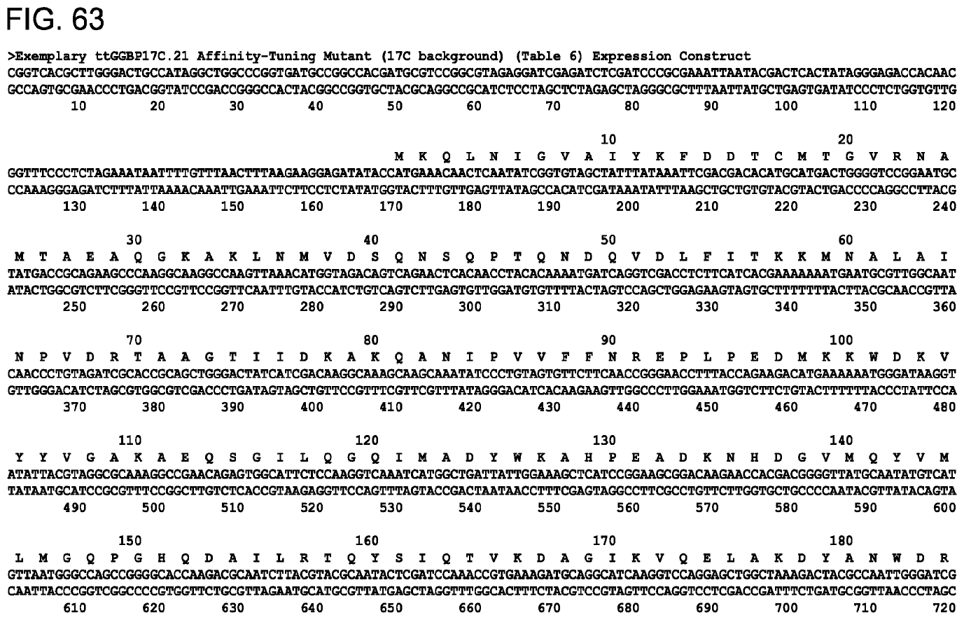

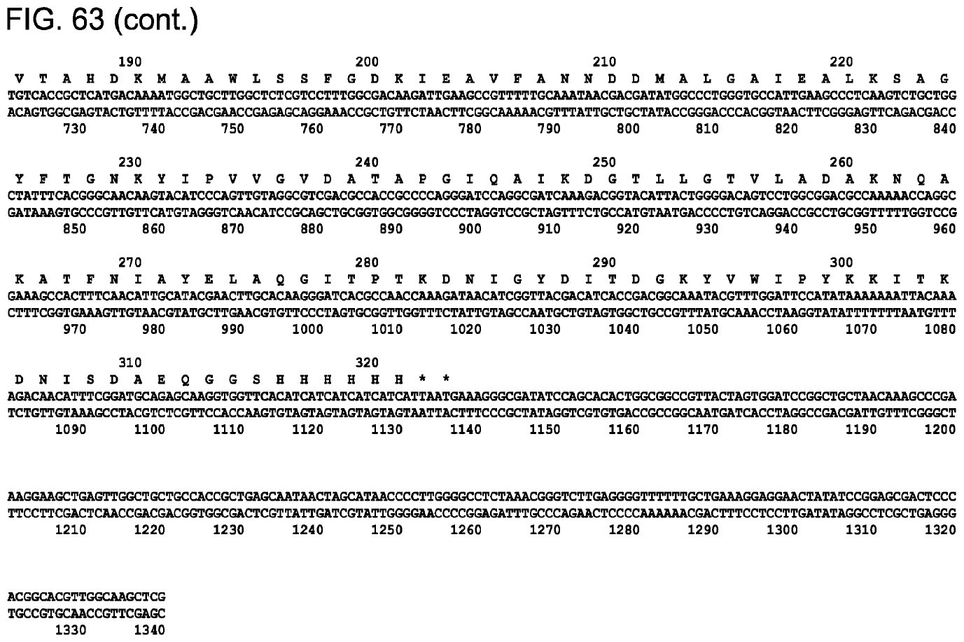

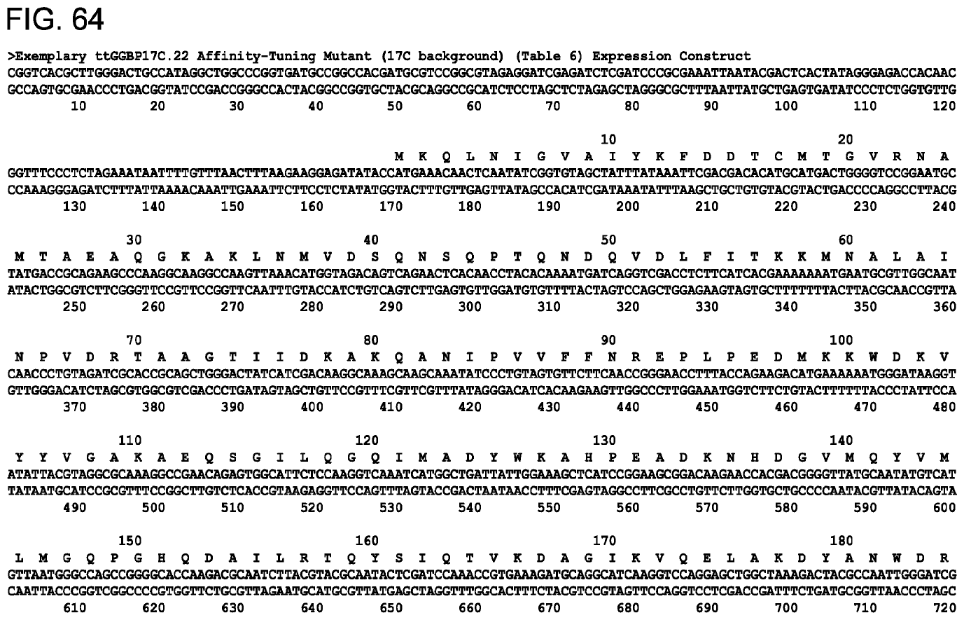

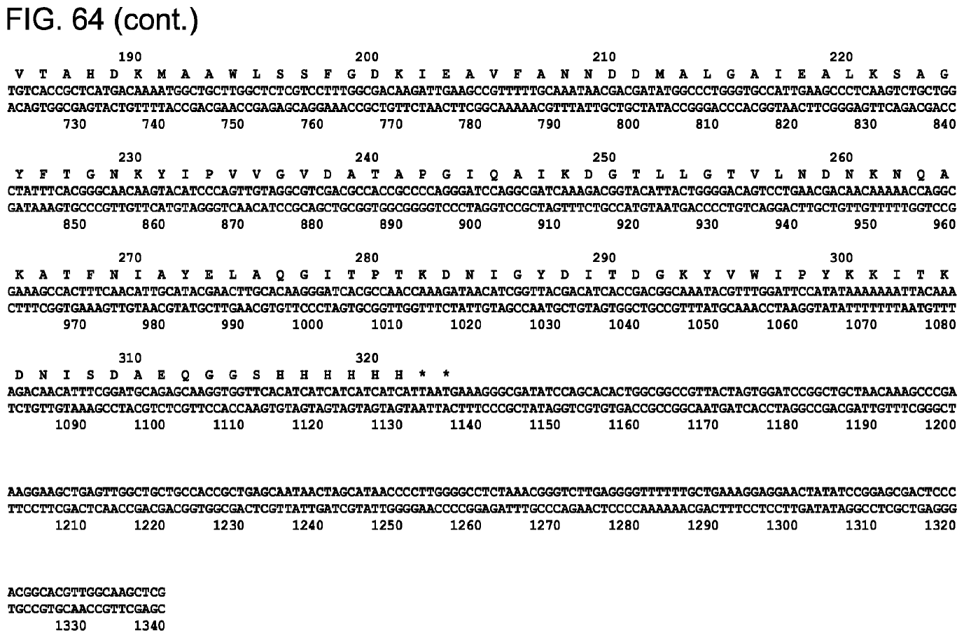

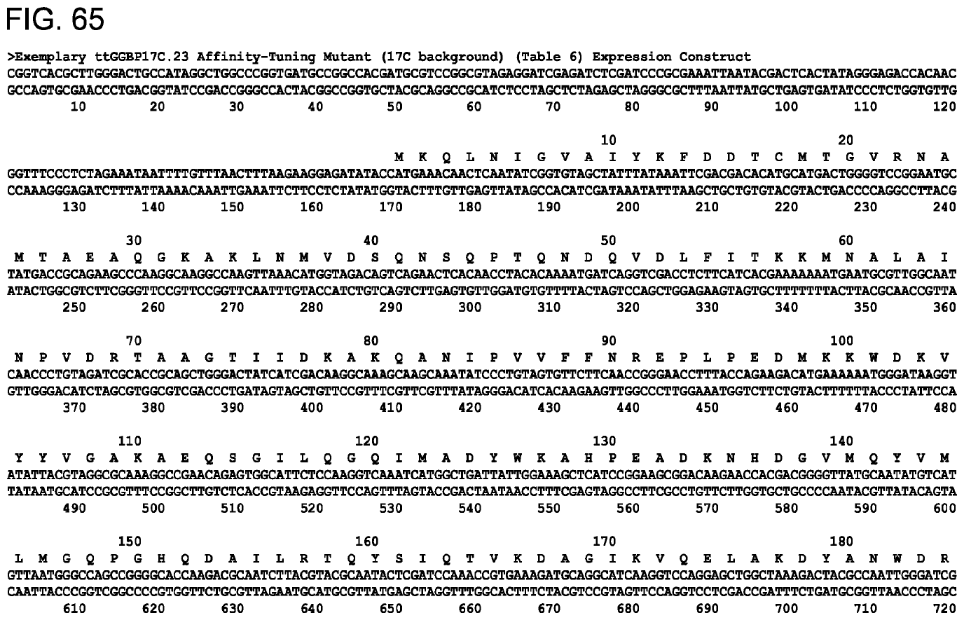

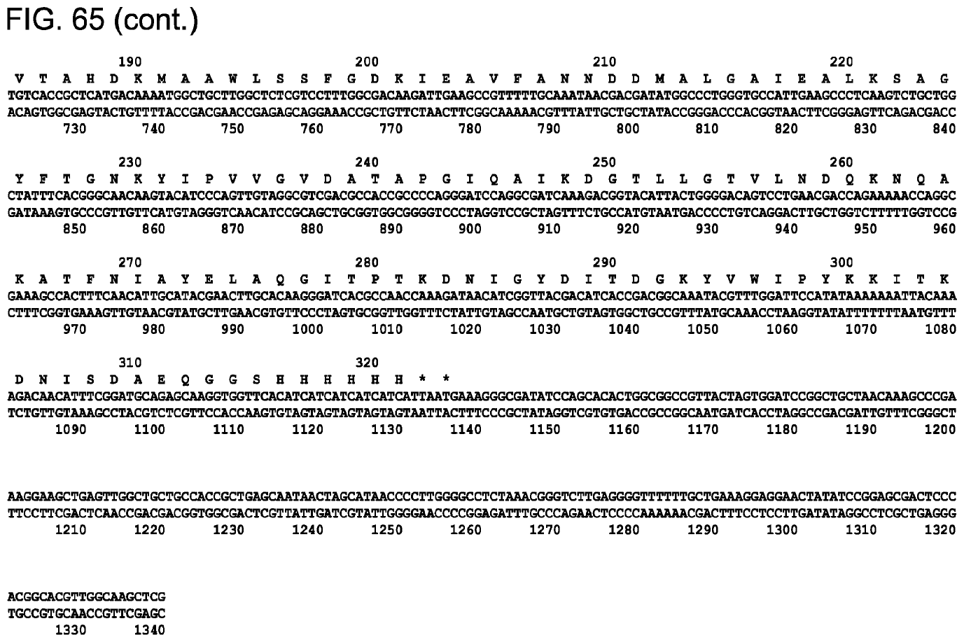

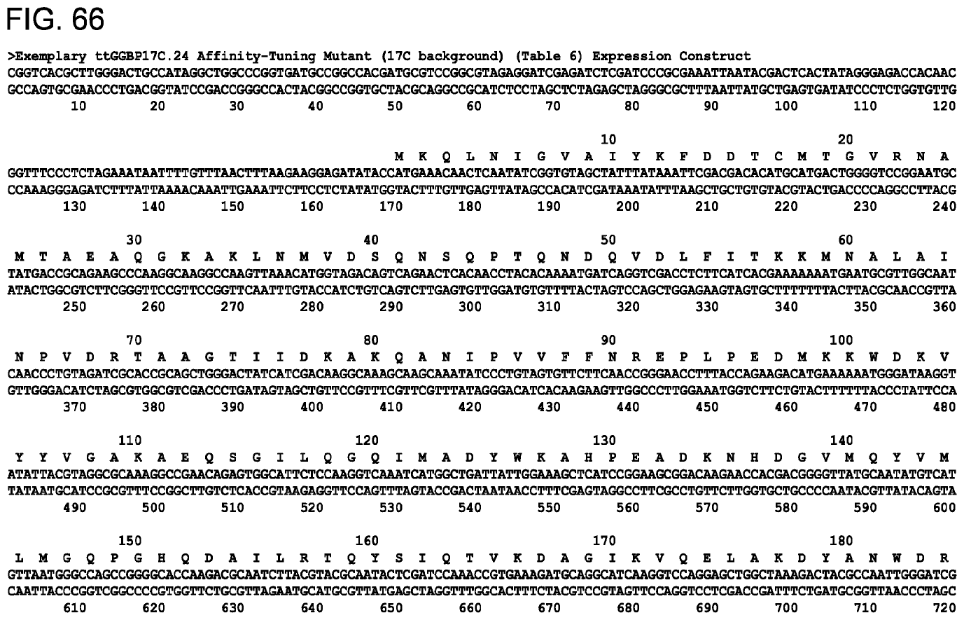

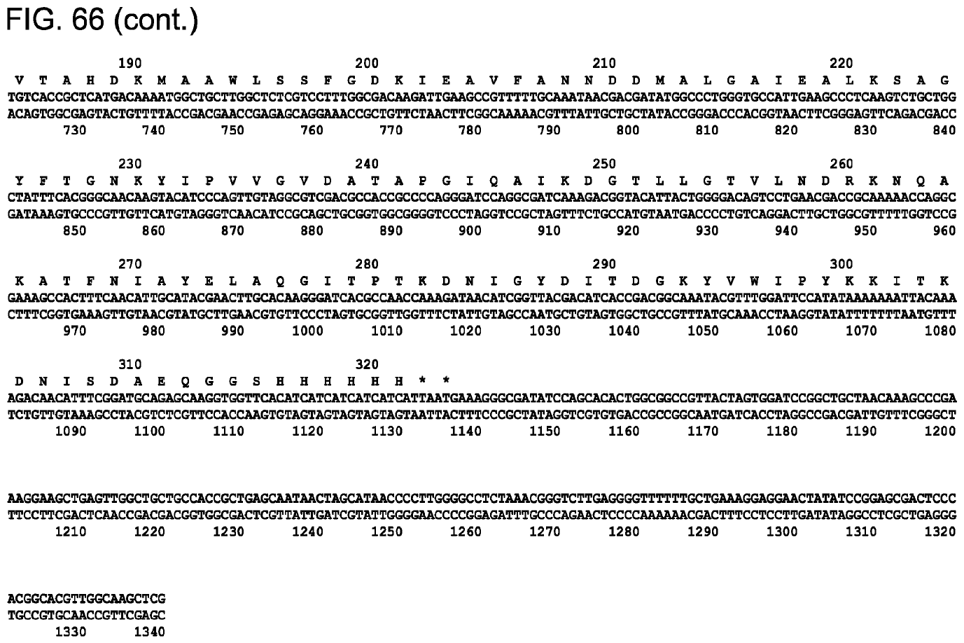

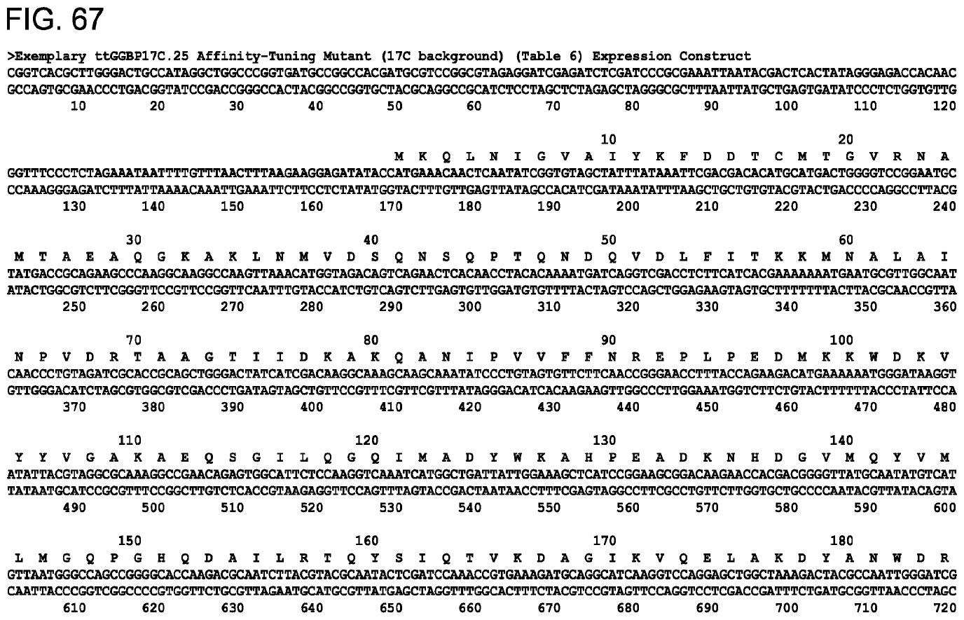

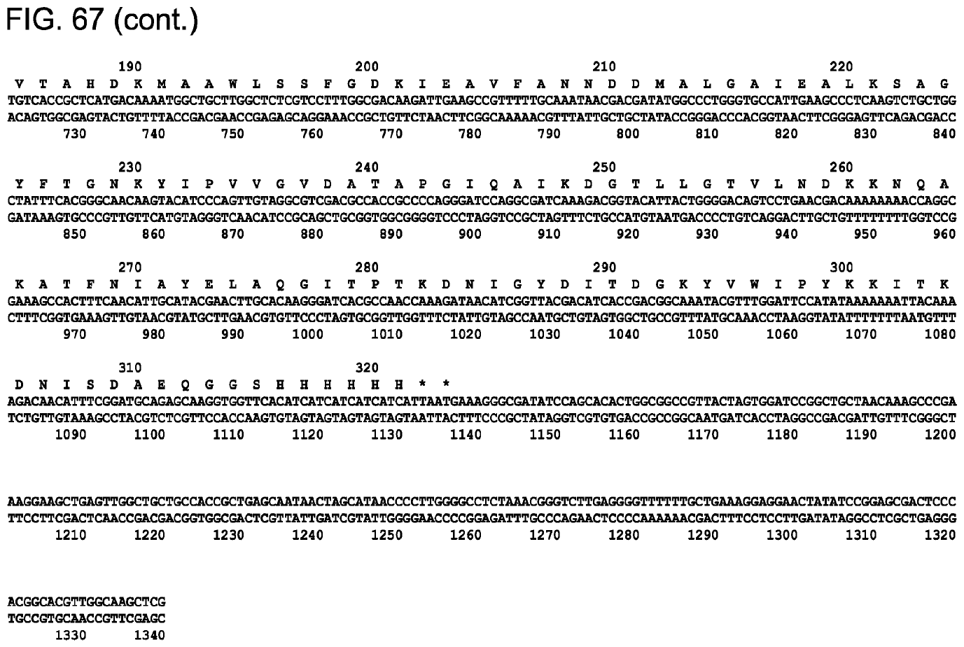

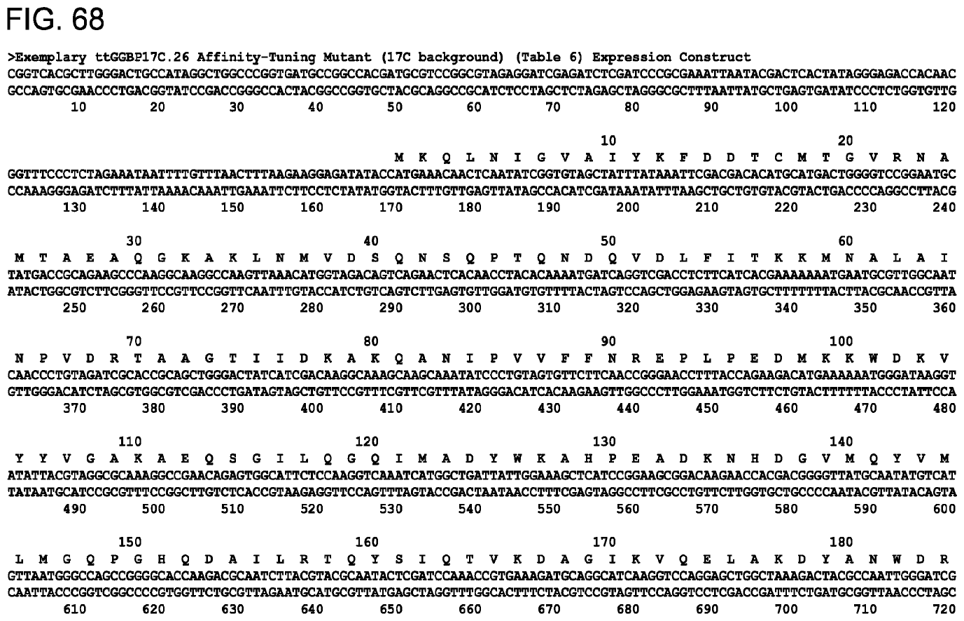

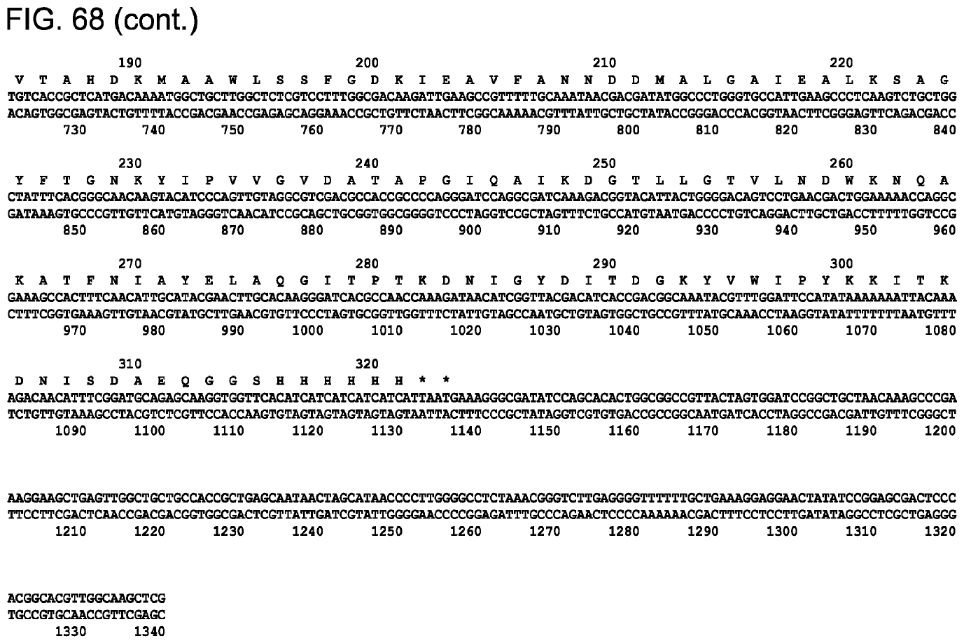

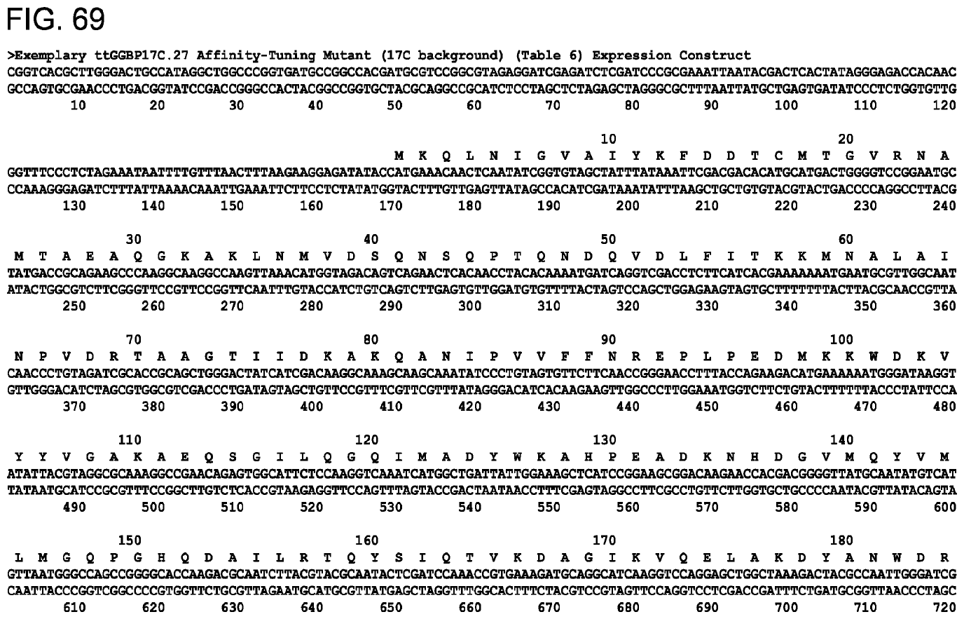

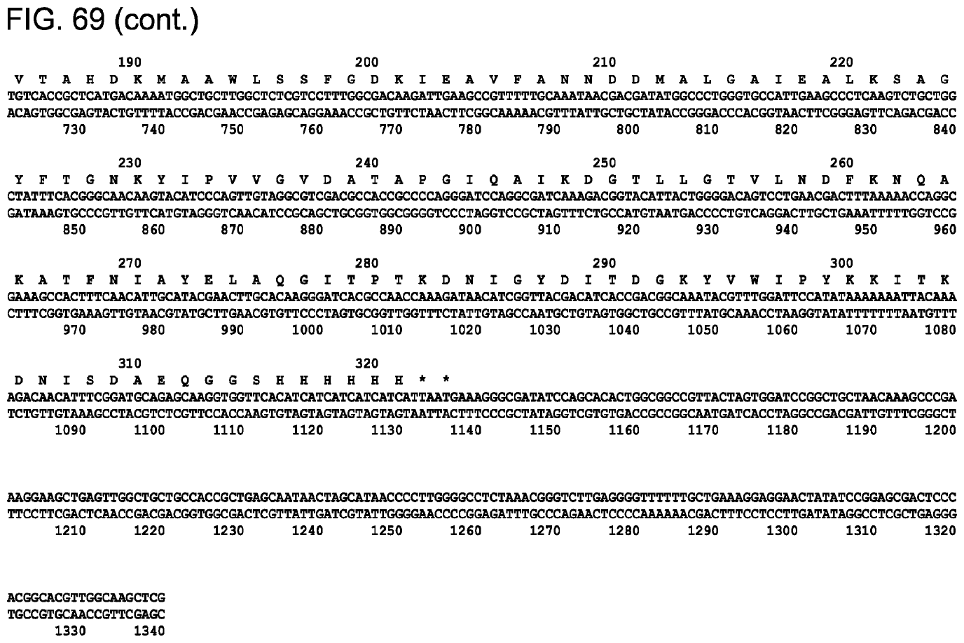

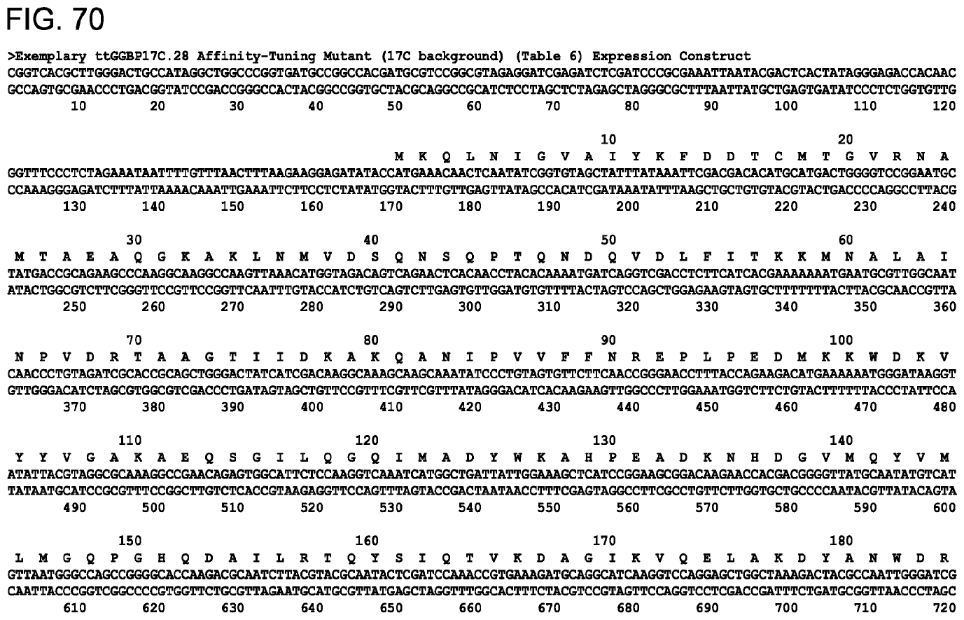

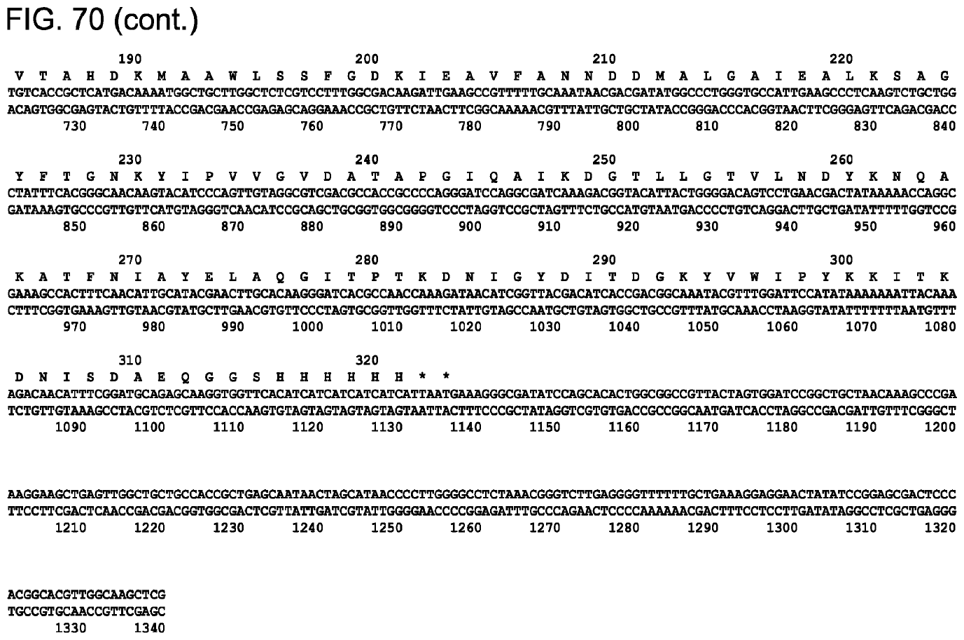

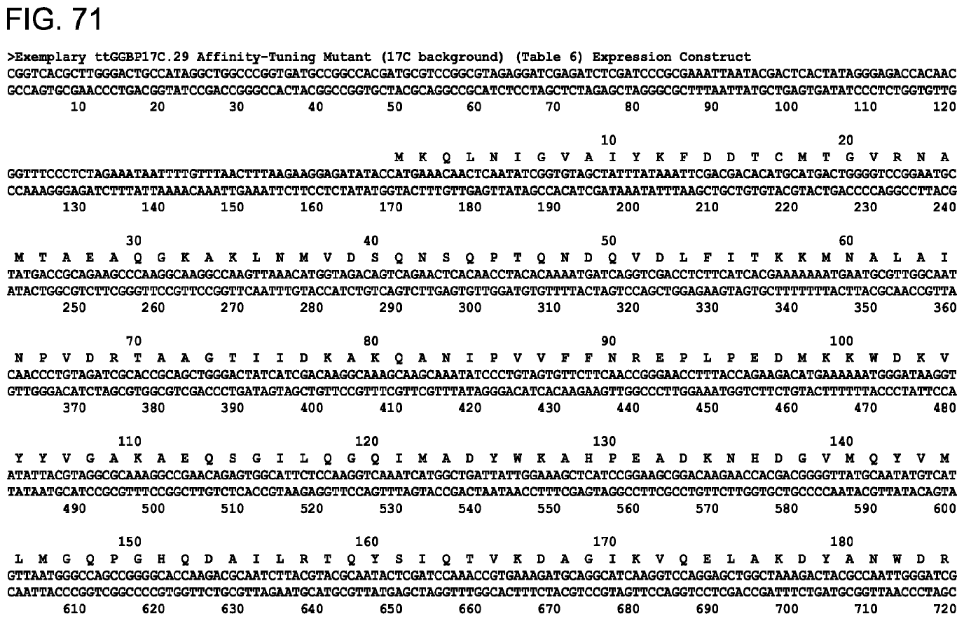

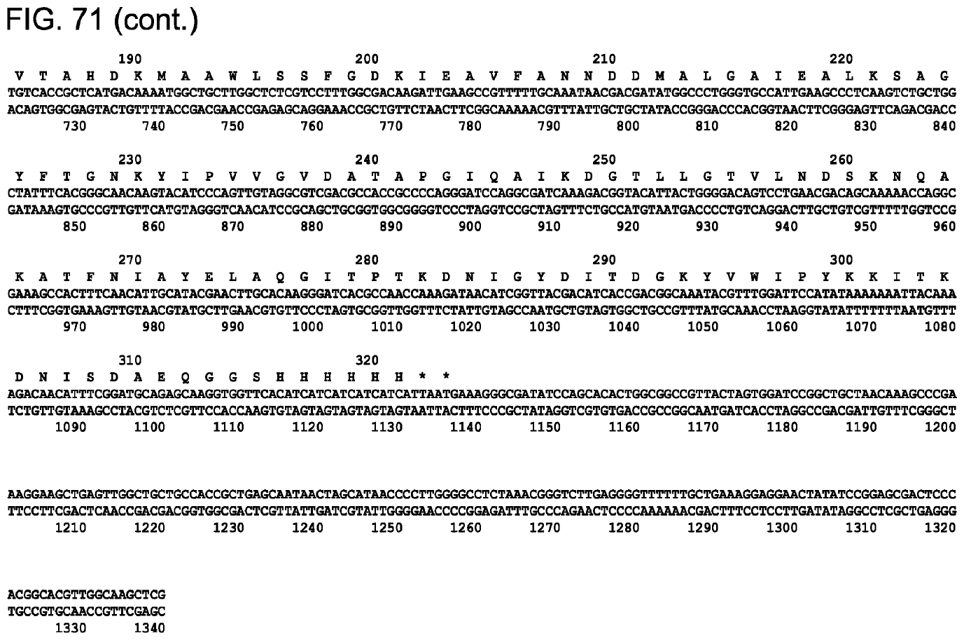

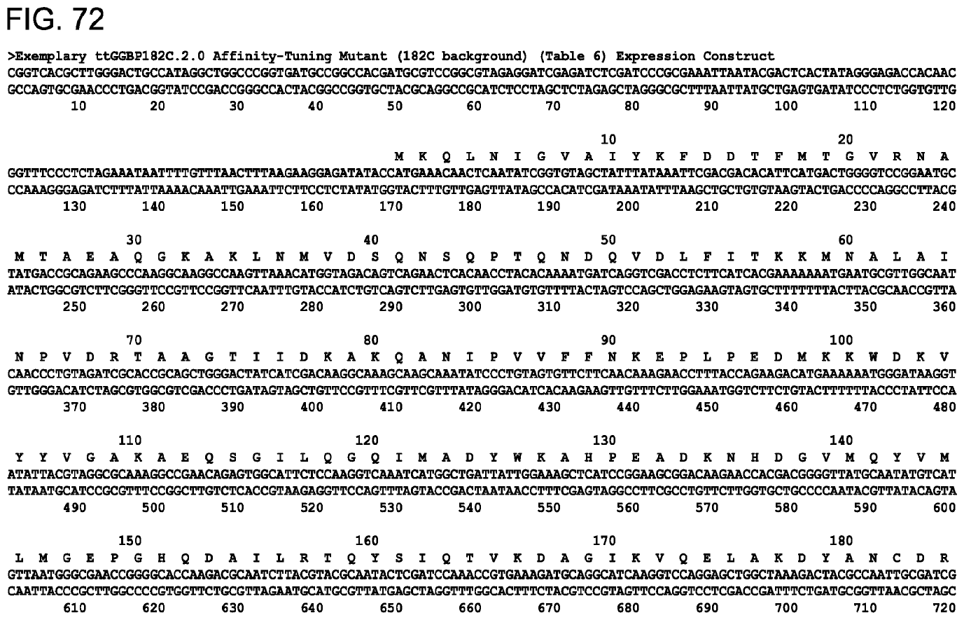

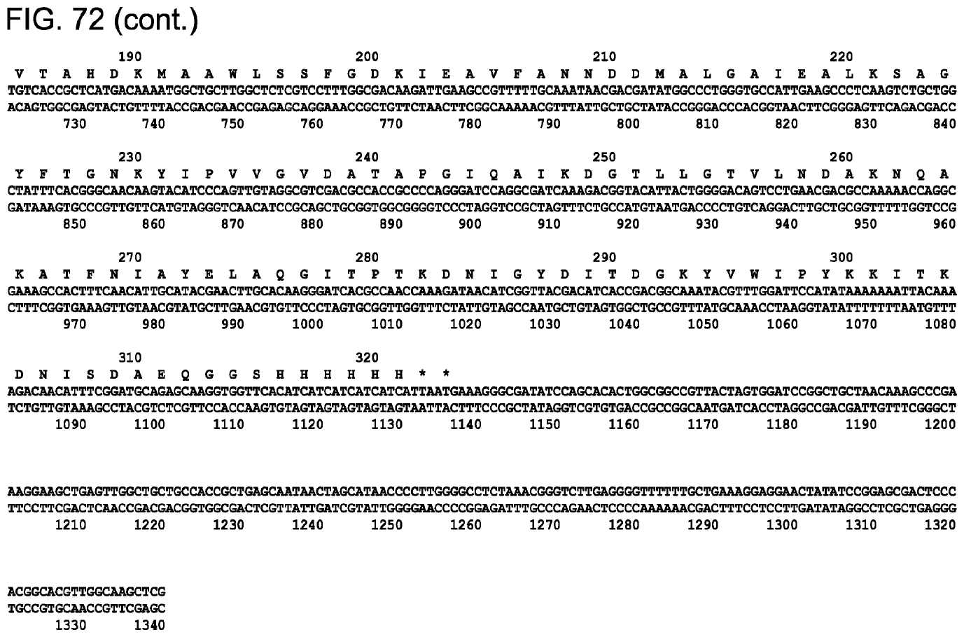

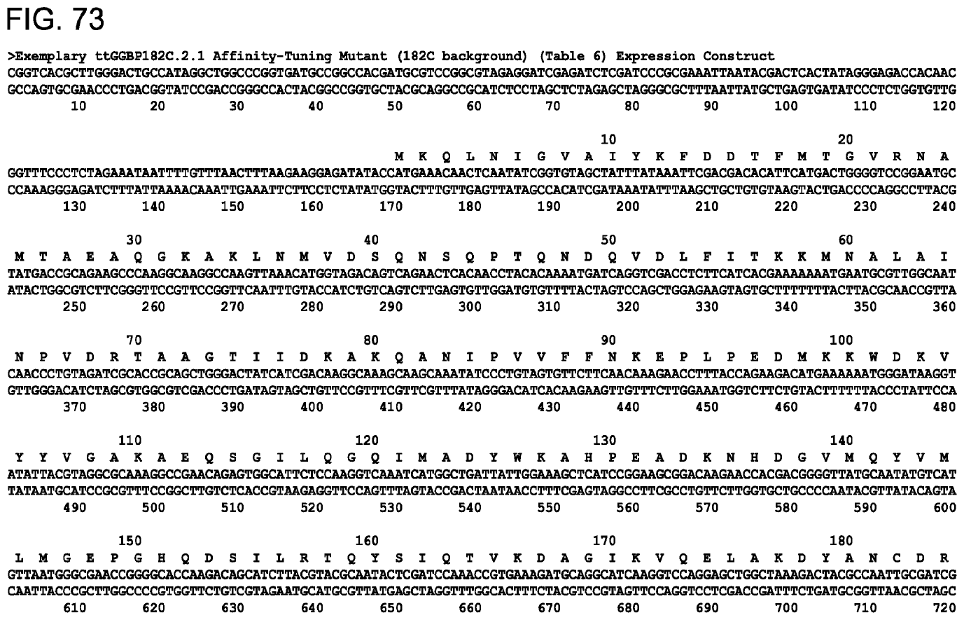

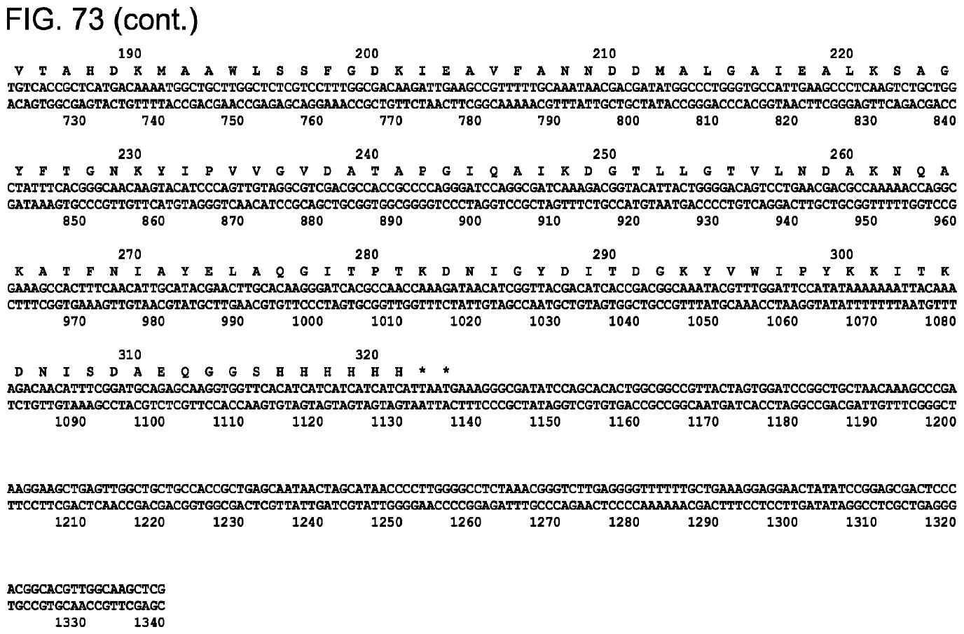

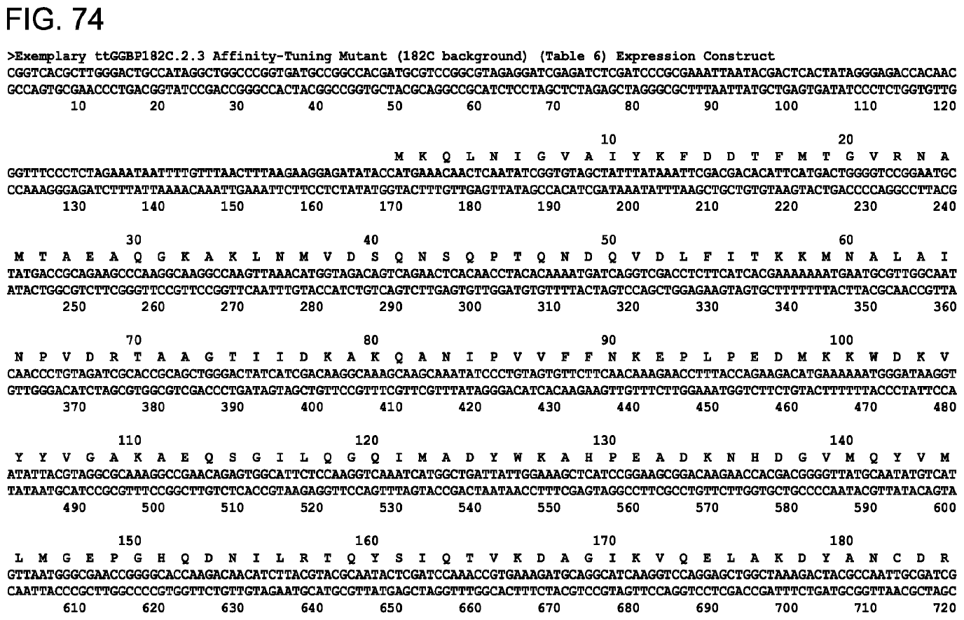

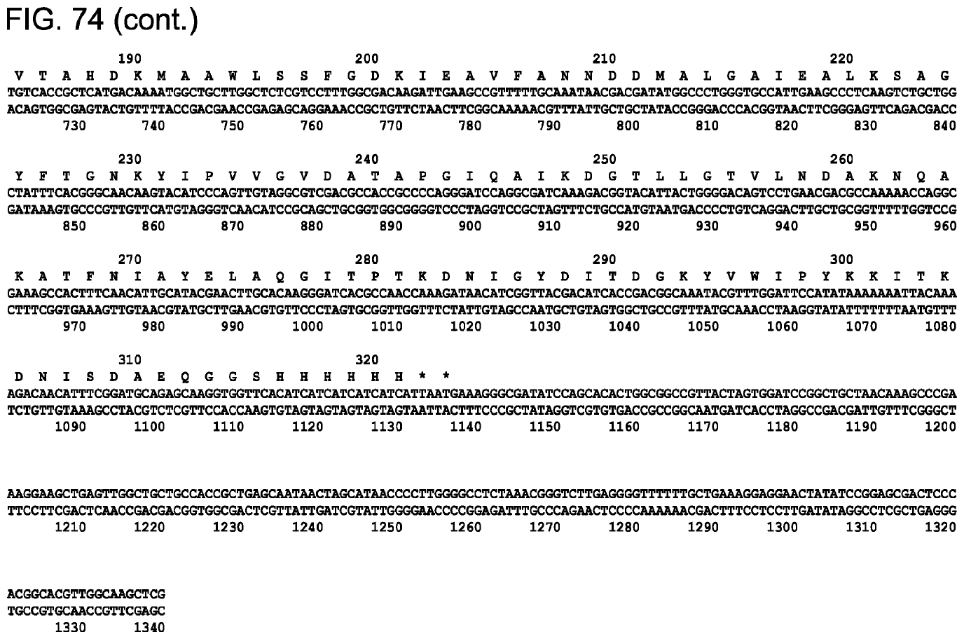

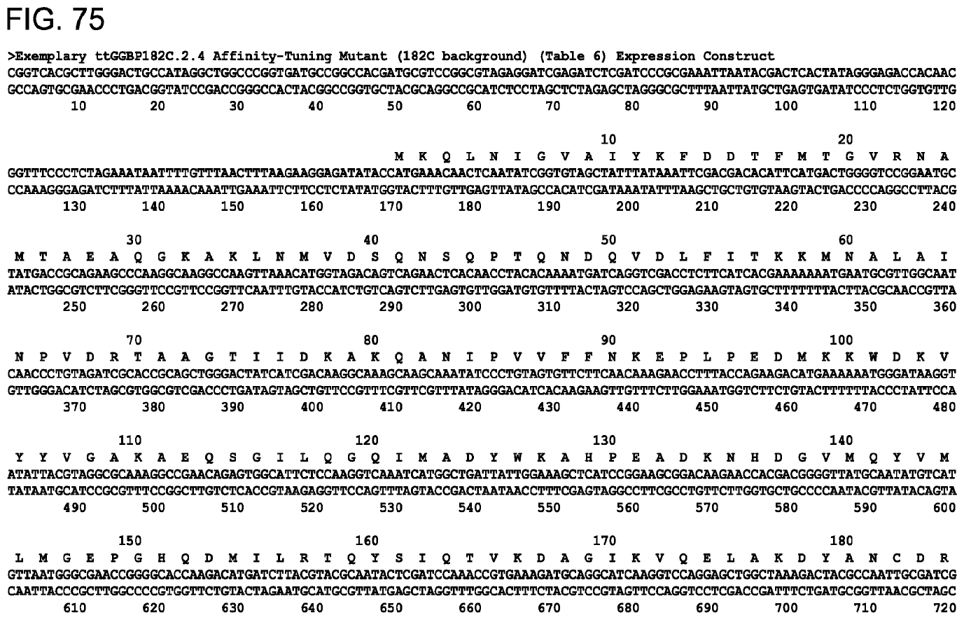

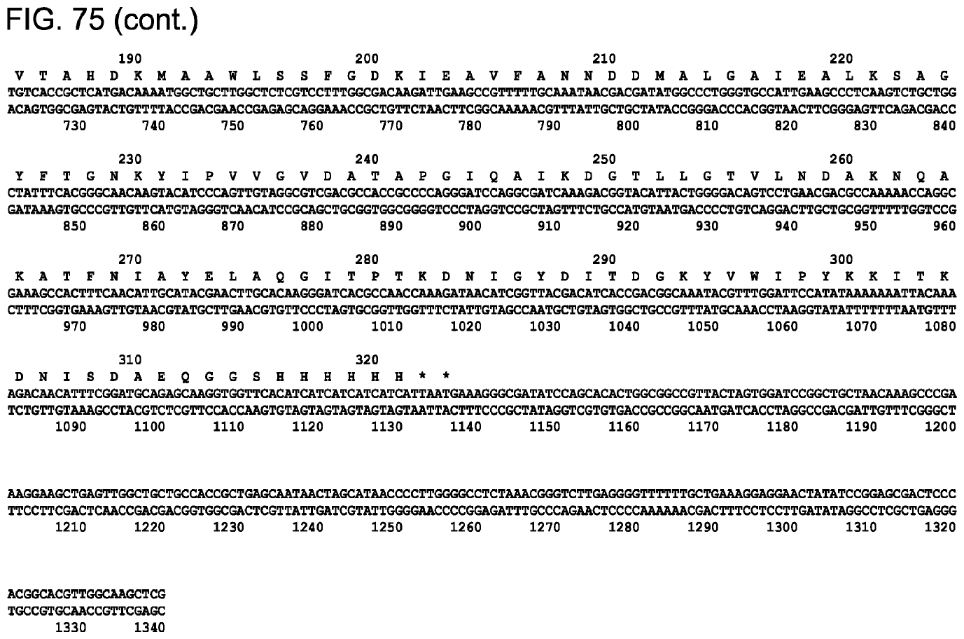

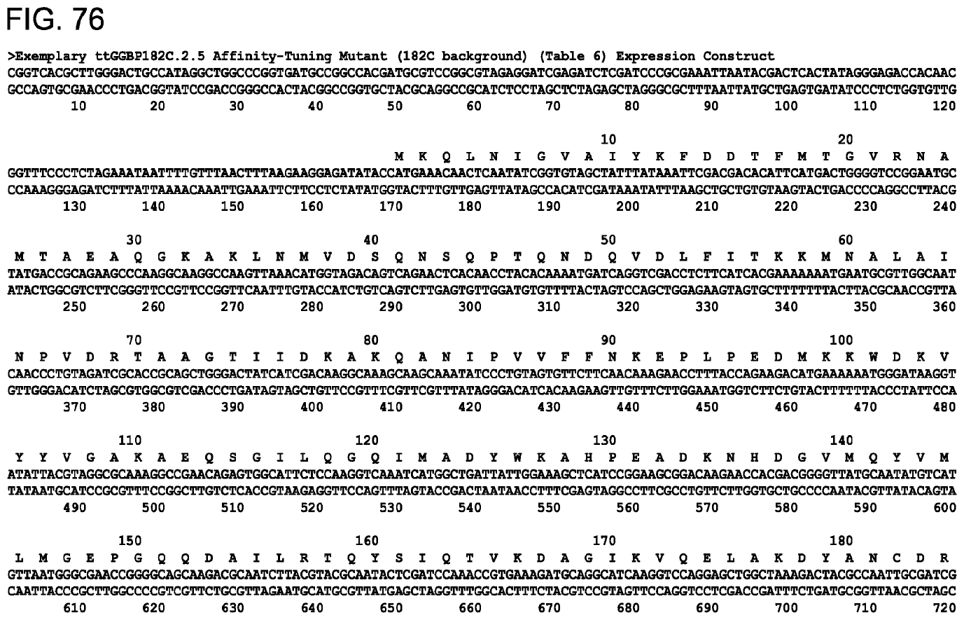

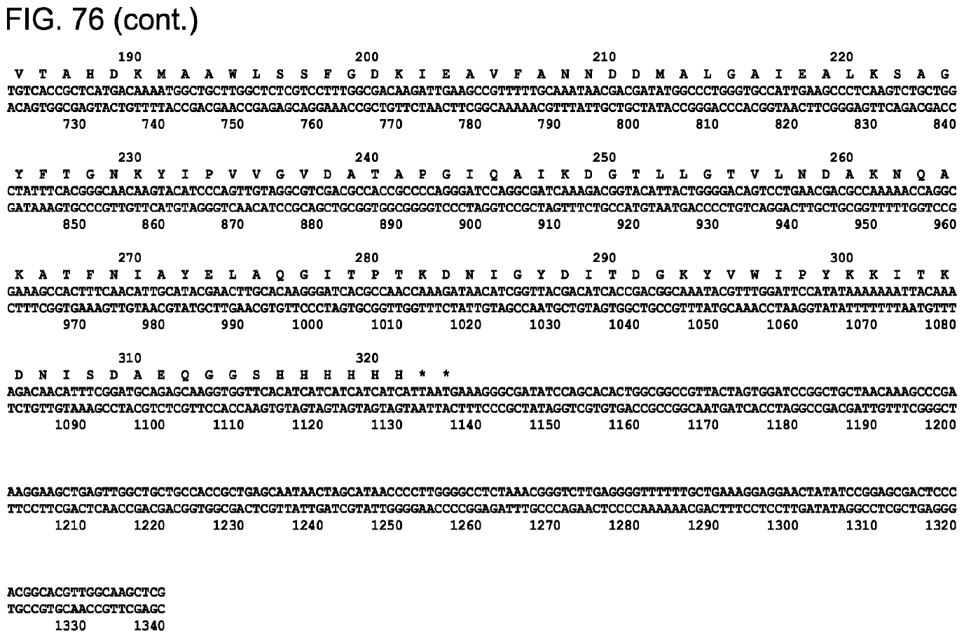

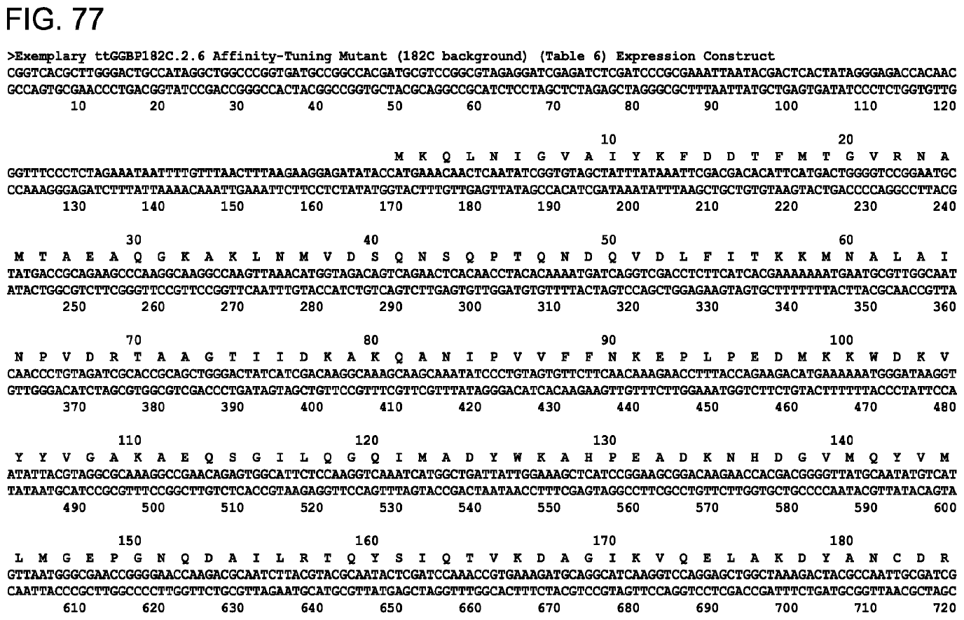

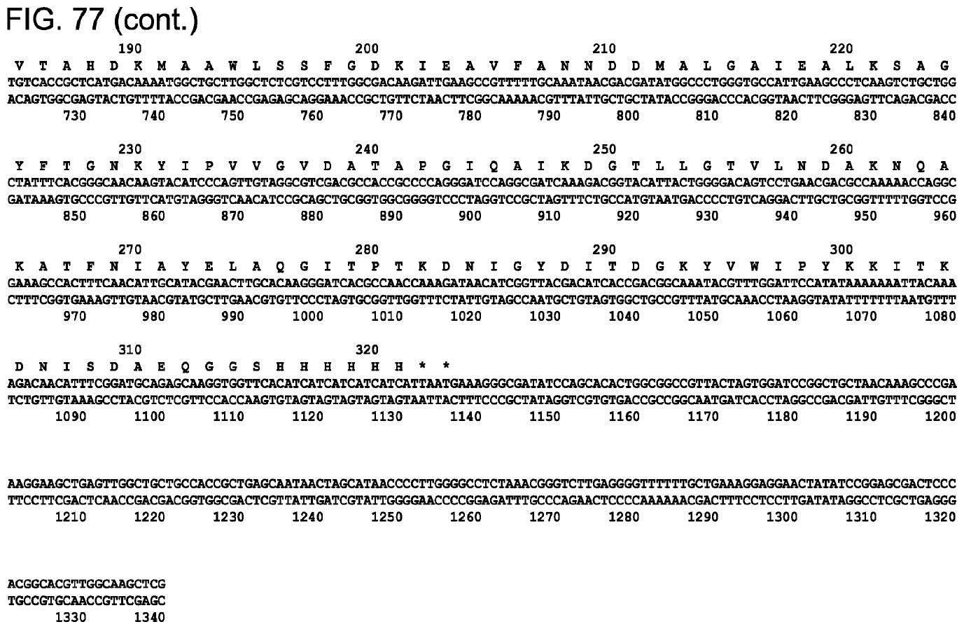

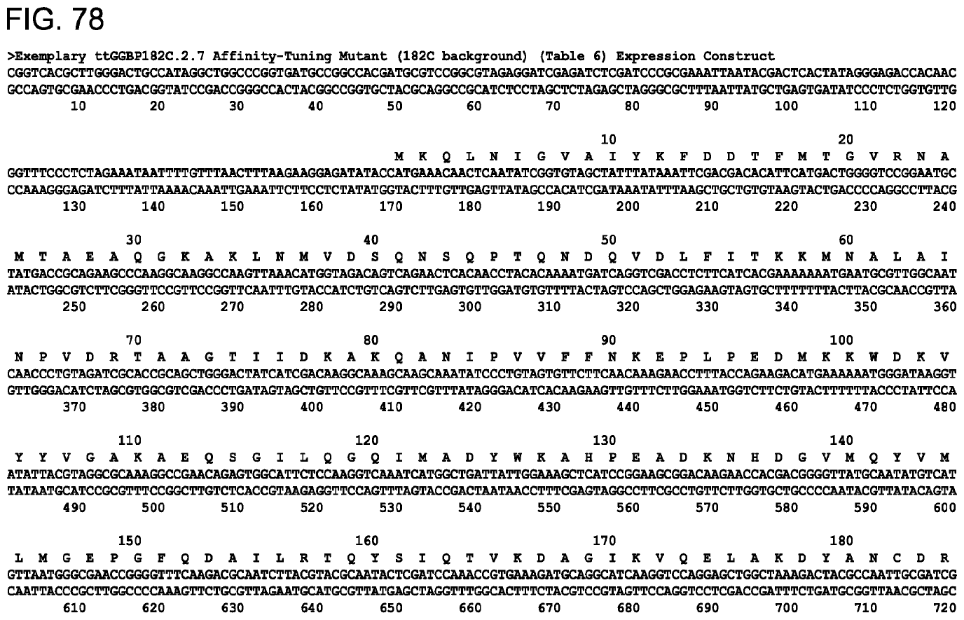

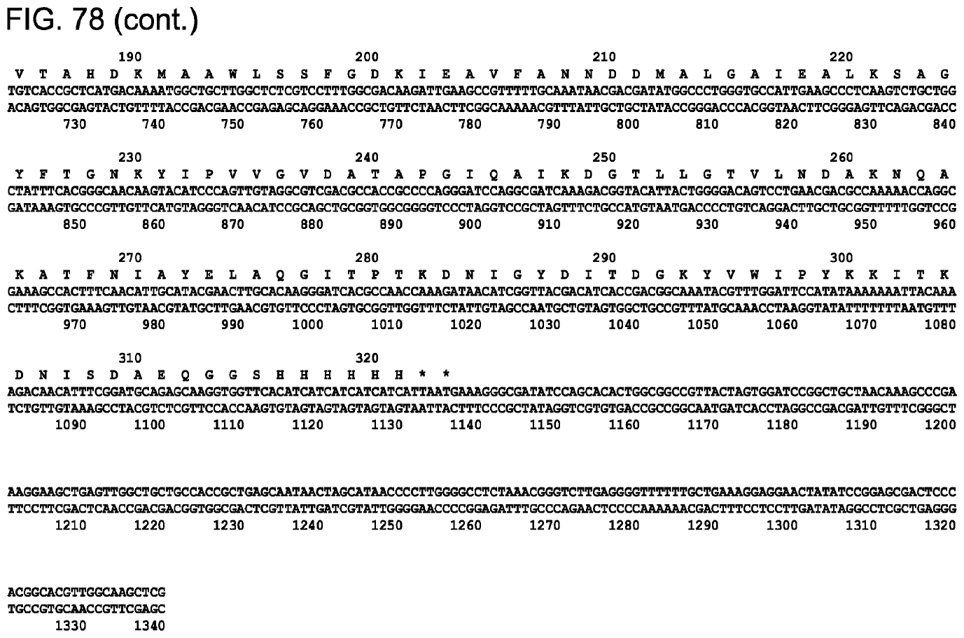

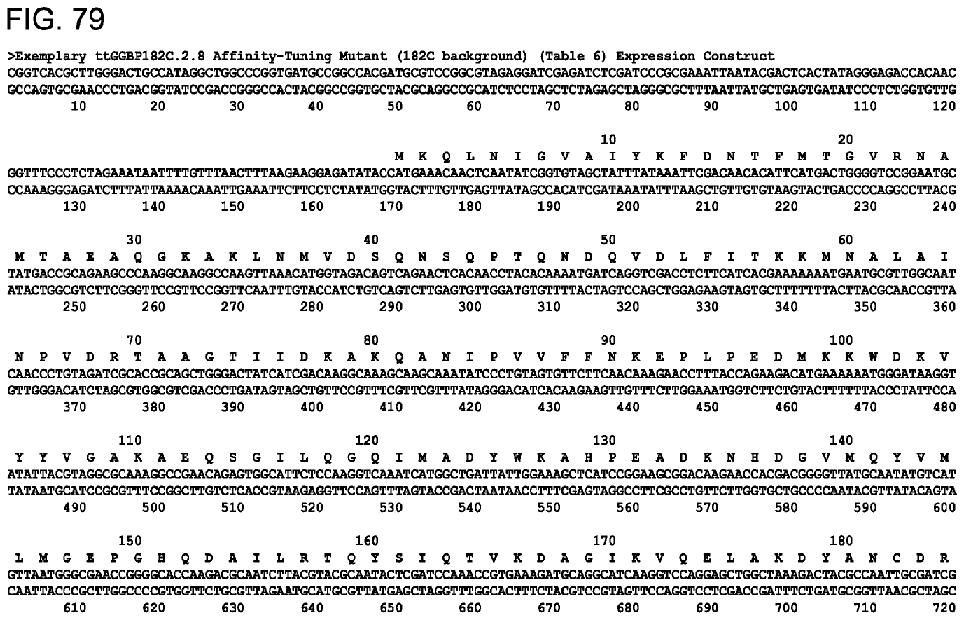

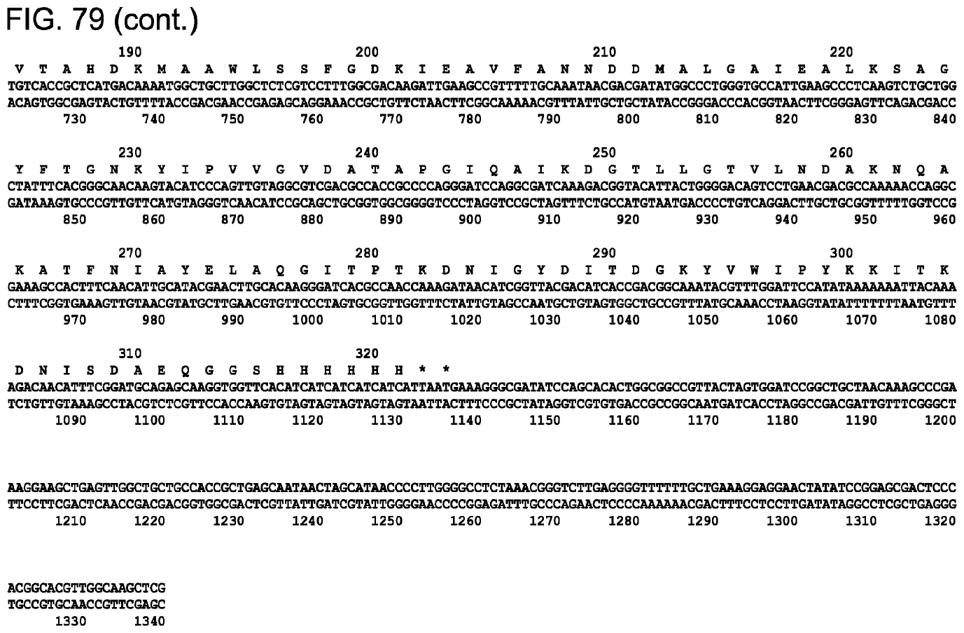

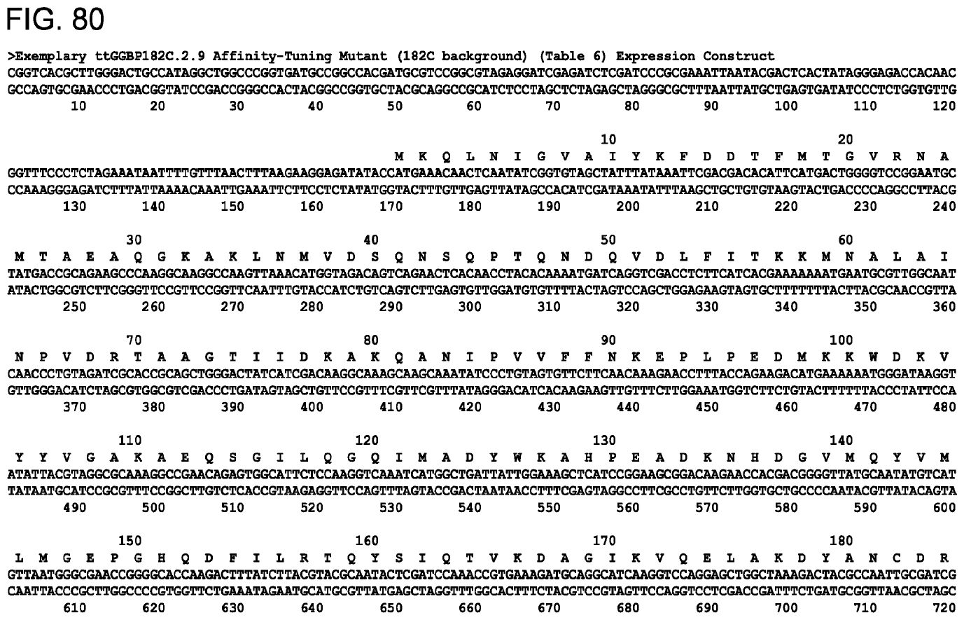

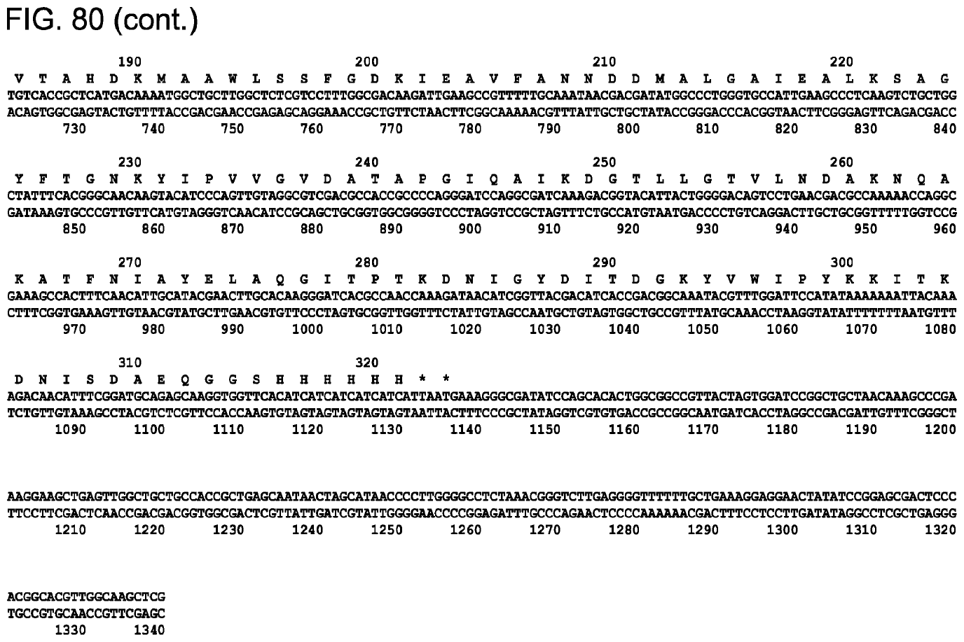

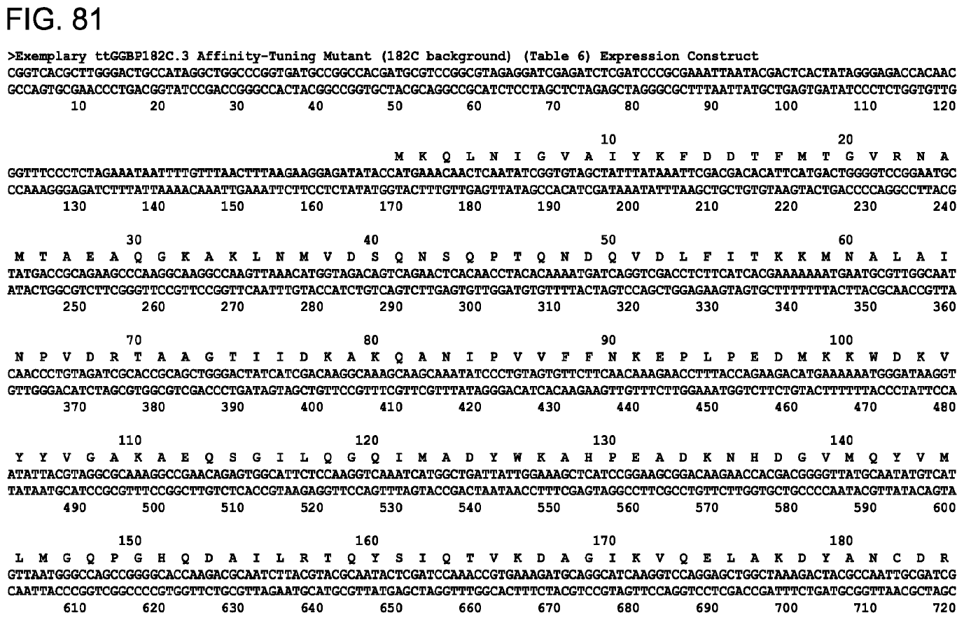

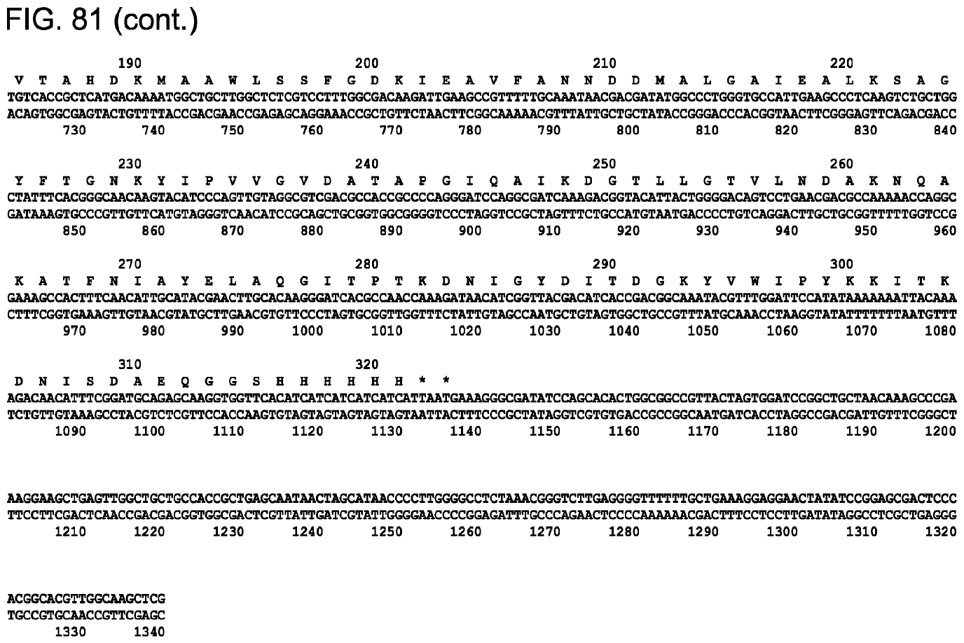

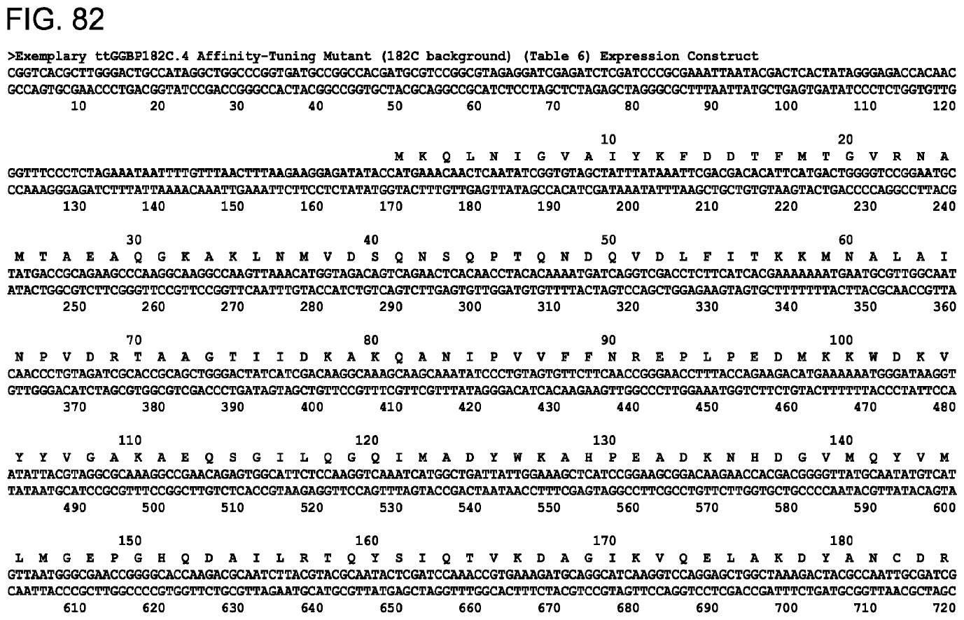

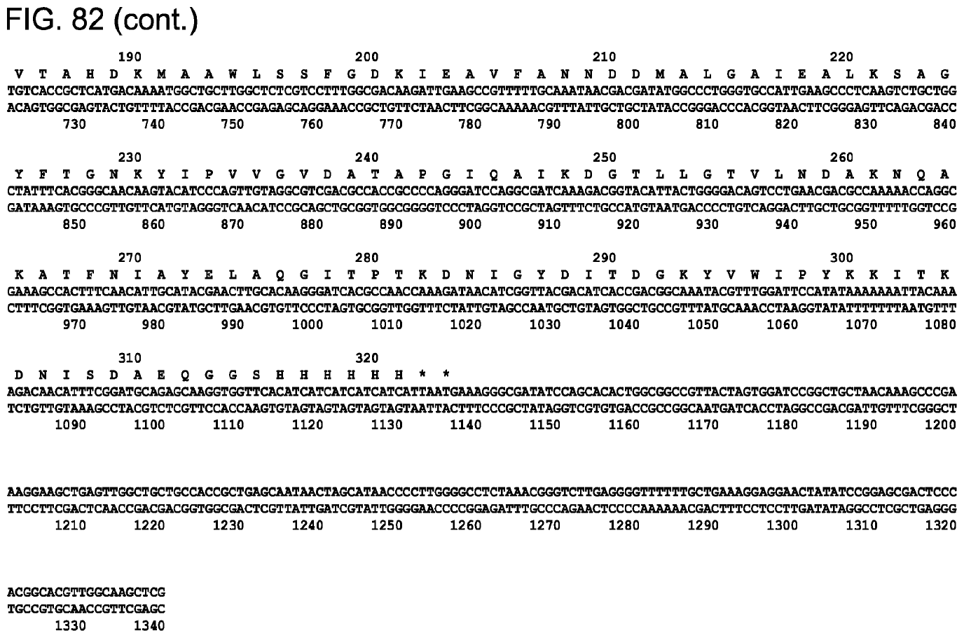

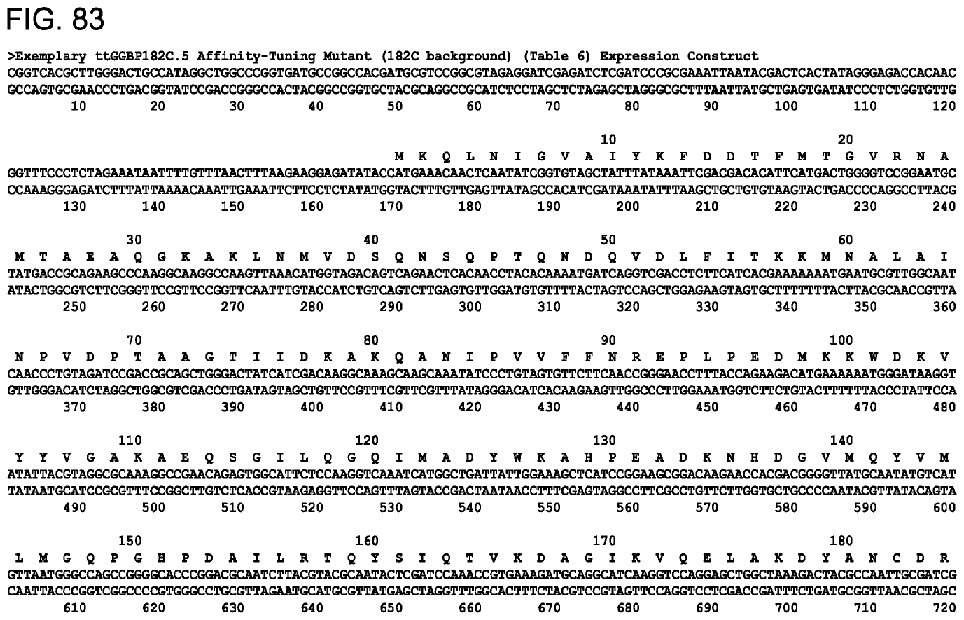

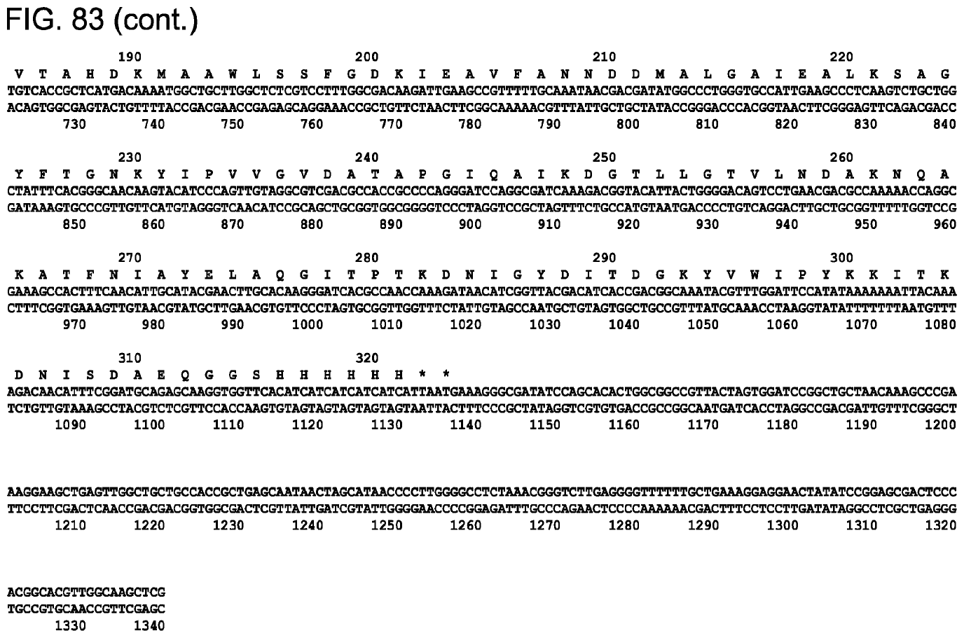

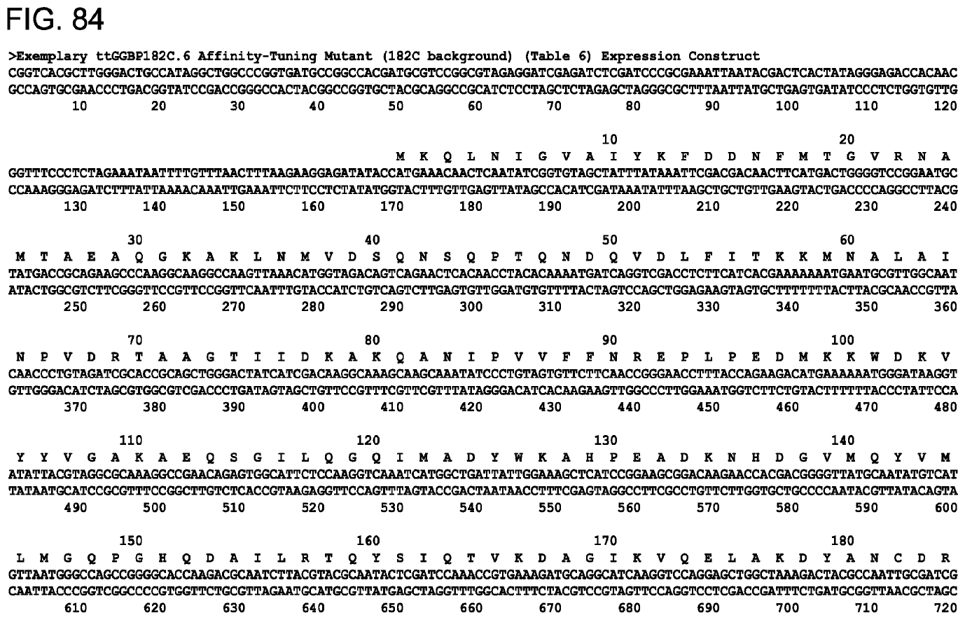

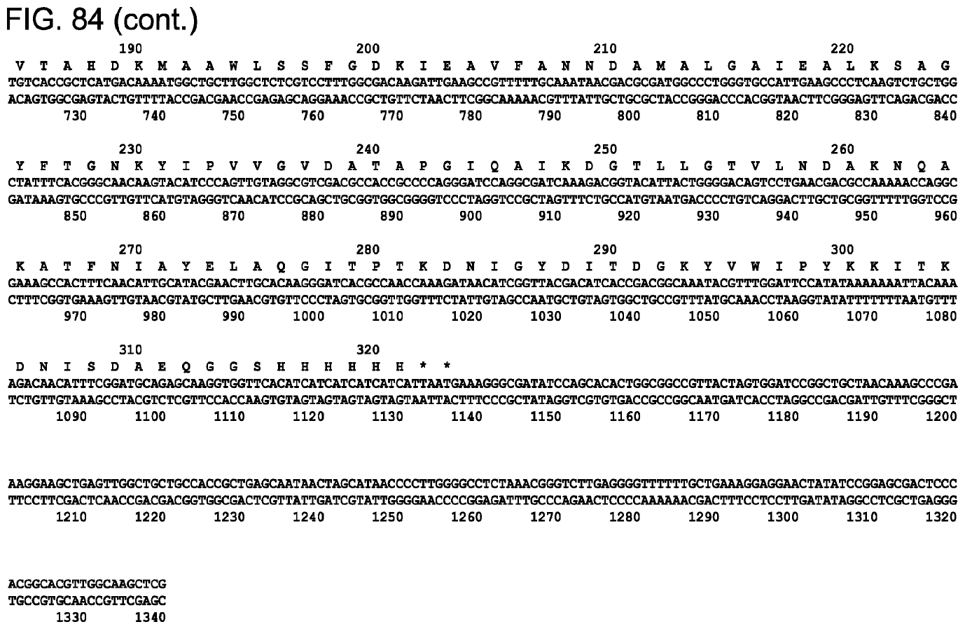

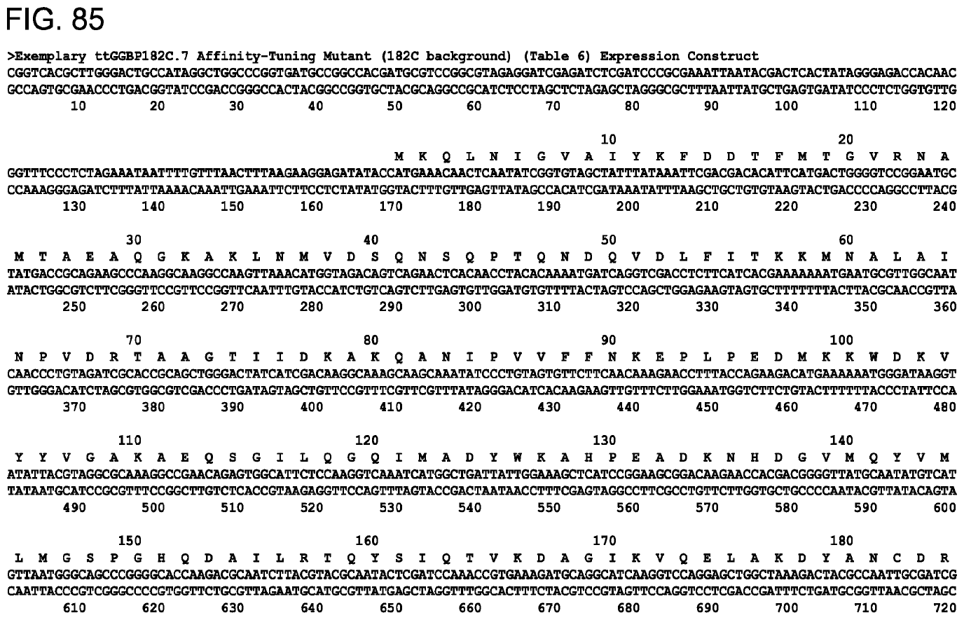

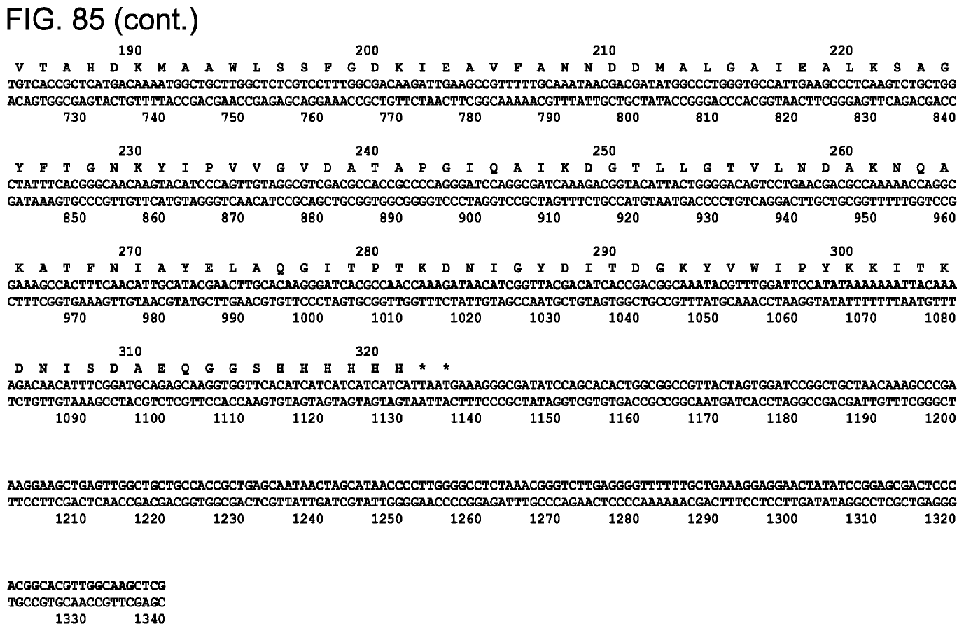

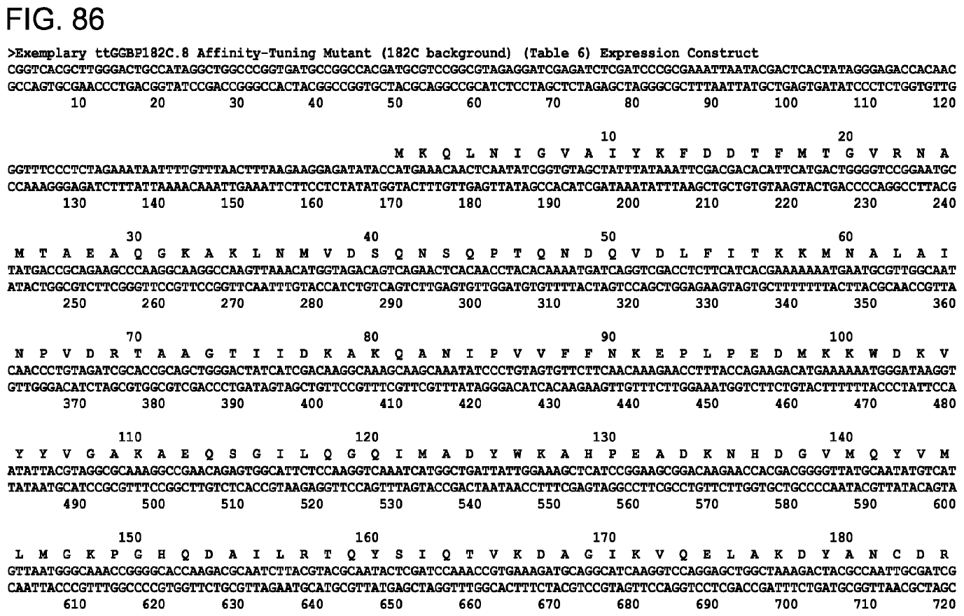

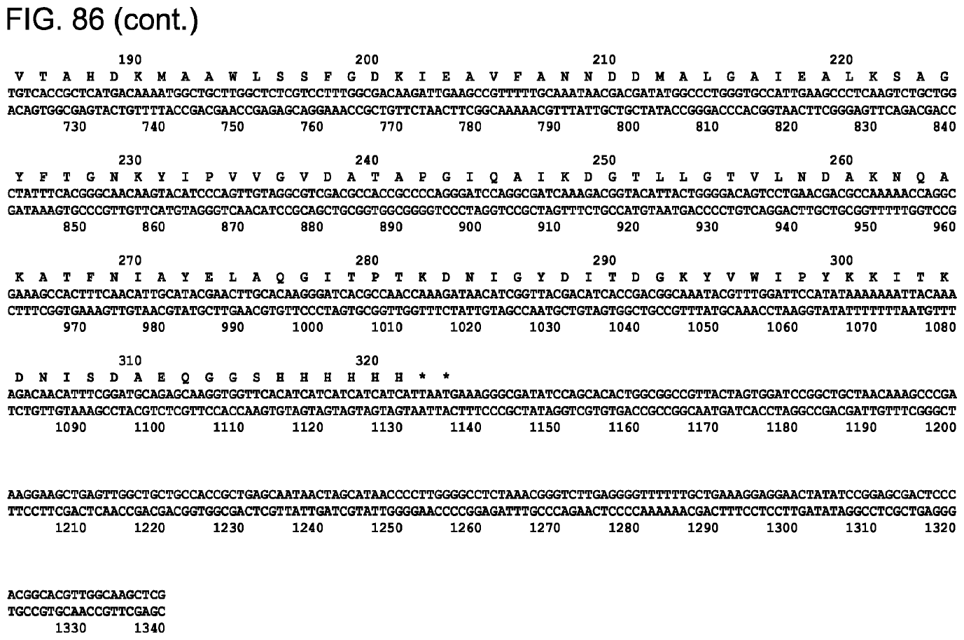

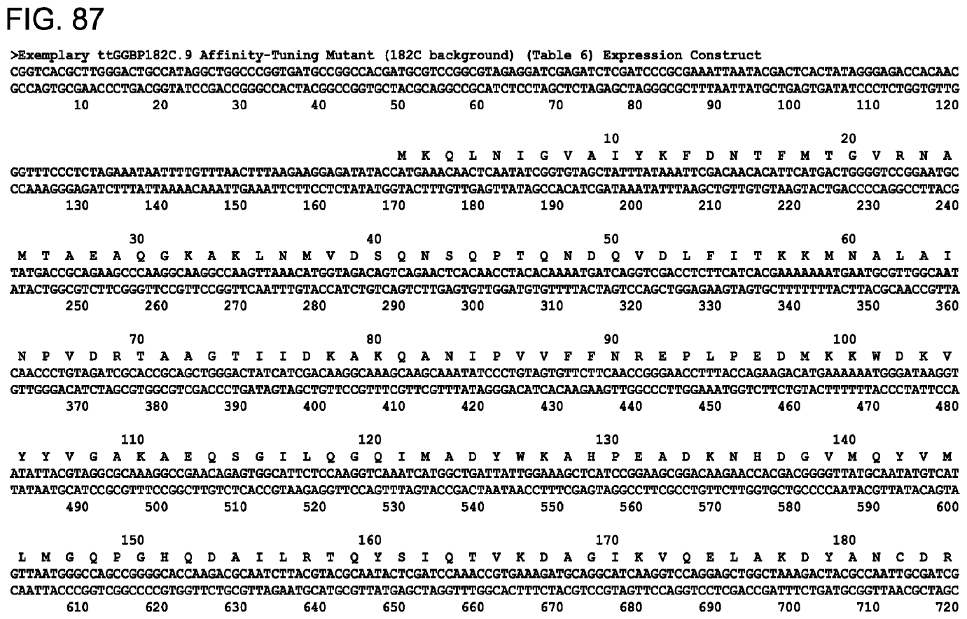

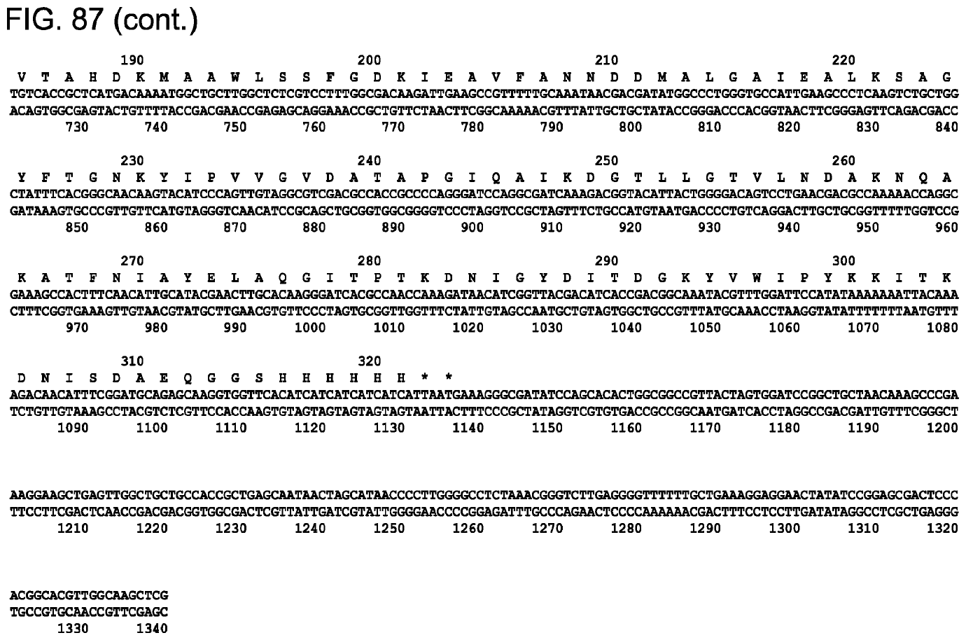

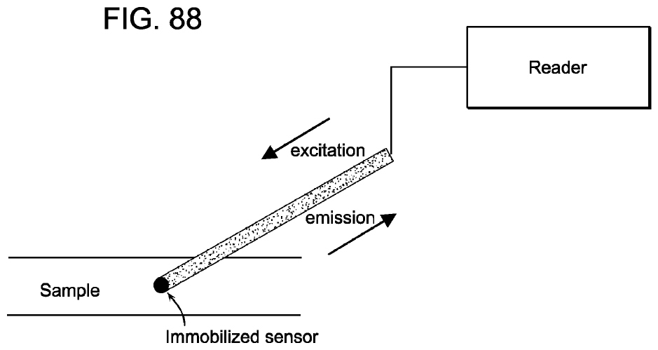

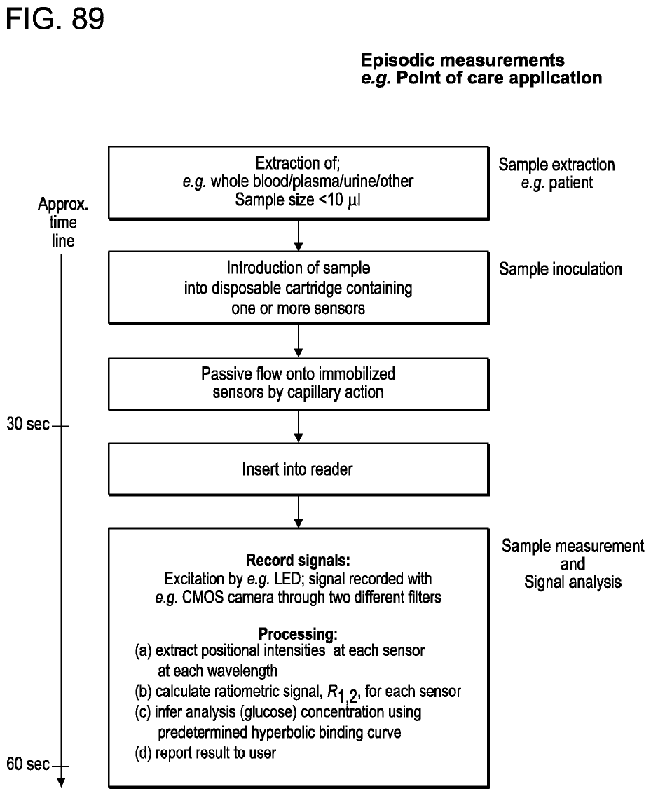

FIGS. 18A-H is a series of graphs showing Glucose dependence of the S₁ and S₂ electronic transition contributions to the fluorescence emission intensity spectra of E. coli GGBP Acrylodan and Badan conjugates. Color schemes, symbols and line representation as in FIGS. 2-4. 16C⋅Acrylodan: (A) emission spectra (hypsochromic) and ratiometric glucose-binding curve (inset: λ₁, 454 nm; λ₂, 540 nm; ^{app}K_{d}, 1.0 mM); (C) singular value decomposition; (E) Gaussian fits and glucose dependence of the S₁ and S₂ populations (inset). 16C⋅Badan: (B) emission spectra (hypsochromic) and ratiometric glucose-binding curve (inset: λ₁, 465 nm; λ₂, 555 nm; ^{app}K_{d}, 0.4 mM); (D) singular value decomposition; (F) Gaussian fits and glucose dependence of the S₁ and S₂ populations (inset). 182C⋅Acrylodan: (G) singular value decomposition; (H) Gaussian fits and glucose dependence of the S₁ and S₂ populations (inset).

FIGS. 19A-F are graphs showing glucose dependence of the absorption spectra of ttGGBP Acrylodan and Badan conjugates that undergo wavelength shifts in their fluorescence emission intensities in response to ligand binding. ttGGBP F17C⋅Badan: (A) Absorption spectra (red, no glucose; purple, saturating glucose; black, intermediate glucose concentrations). Hypsochromic response was modeled by fit of a Langmuir ligand-binding isotherm (equation 1) to ratiometric analysis of the absorption intensities (inset: λ₁, 390 nm; λ₂, 340 nm; ^{app}K_{d}, 1.7 mM); (C) singular value decomposition (components: black, C₁; gray, C₂); (E) Fit of Gaussian representations of electronic transitions to absorption spectra (dashed black line, apo-protein; solid black line, glucose complex) of G₁ (386 nm green; apo-protein, dashed; glucose complex, solid) and G₂ (359 nm, blue; apo-protein, dashed; glucose complex, solid) ground state electronic transitions. Inset, glucose dependence of population fractions at each glucose titration (black circles) of the G₁ and G₂ transitions fit to Langmuir binding isotherms (solid lines) with ^{app}K_{d} values constrained to be the same for both populations (residuals, thin lines). ttGGBP F182C⋅Acrylodan (color scheme, line representations, symbols, as above): (B) Absorption spectra (bathochromic response) and glucose binding (inset: λ₁, 375 nm; λ₂, 430 nm; ^{app}K_{d}, 1.1 mM; ^{true}K_{d}, 1.9 mM); (D) singular value decomposition; (F) G₁ and G₂ electronic transitions and populations. The residuals indicate that the maximal wavelength of the G₁ and G₂ transitions is different in the apo-protein and glucose (also see legend to FIG. 17).

FIG. 20 shows the sequence of an exemplary csaGGBP (Table 2) expression construct (SEQ ID NO: 105).

FIG. 21 shows the sequence of an exemplary bprGGBP (Table 2) expression construct (SEQ ID NO: 106).

FIG. 22 shows the sequence of an exemplary rinGGBP_A (Table 2) expression construct (SEQ ID NO: 107).

FIG. 23 shows the sequence of an exemplary fprGGBP (Table 2) expression construct (SEQ ID NO: 108).

FIG. 24 shows the sequence of an exemplary cljGGBP (Table 2) expression construct (SEQ ID NO: 109).

FIG. 25 shows the sequence of an exemplary cauGGBP (Table 2) expression construct (SEQ ID NO: 110).

FIG. 26 shows the sequence of an exemplary rinGGBP_B (Table 2) expression construct (SEQ ID NO: 111).

FIG. 27 shows the sequence of an exemplary erhGGBP (Table 2) expression construct (SEQ ID NO: 112).

FIG. 28 shows the sequence of an exemplary ereGGBP (Table 2) expression construct (SEQ ID NO: 113).

FIG. 29 shows the sequence of an exemplary ttGGBP (Table 2) expression construct (SEQ ID NO: 114).

FIG. 30 shows the sequence of an exemplary cobGGBP (Table 2) expression construct (SEQ ID NO: 115).

FIG. 31 shows the sequence of an exemplary chyGGBP (Table 2) expression construct (SEQ ID NO: 116).

FIG. 32 shows the sequence of an exemplary pspGGBP (Table 2) expression construct (SEQ ID NO: 117).

FIG. 33 shows the sequence of an exemplary ttGGBP11C Cysteine Scan Mutant (Table 3) expression construct (SEQ ID NO: 118).

FIG. 34 shows the sequence of an exemplary ttGGBP16C Cysteine Scan Mutant (Table 3) expression construct (SEQ ID NO: 119).

FIG. 35 shows the sequence of an exemplary ttGGBP17C Cysteine Scan Mutant (Table 3) expression construct (SEQ ID NO: 120).

FIG. 36 shows the sequence of an exemplary ttGGBP42C Cysteine Scan Mutant (Table 3) expression construct (SEQ ID NO: 121).

FIG. 37 shows the sequence of an exemplary ttGGBP67C Cysteine Scan Mutant (Table 3) expression construct (SEQ ID NO: 122).

FIG. 38 shows the sequence of an exemplary ttGGBP91C Cysteine Scan Mutant (Table 3) expression construct (SEQ ID NO: 123).

FIG. 39 shows the sequence of an exemplary ttGGBP92C Cysteine Scan Mutant (Table 3) expression construct (SEQ ID NO: 124).

FIG. 40 shows the sequence of an exemplary ttGGBP111C Cysteine Scan Mutant (Table 3) expression construct (SEQ ID NO: 125).

FIG. 41 shows the sequence of an exemplary ttGGBP148C Cysteine Scan Mutant (Table 3) expression construct (SEQ ID NO: 126).

FIG. 42 shows the sequence of an exemplary ttGGBP151C Cysteine Scan Mutant (Table 3) expression construct (SEQ ID NO: 127).

FIG. 43 shows the sequence of an exemplary ttGGBP152C Cysteine Scan Mutant (Table 3) expression construct (SEQ ID NO: 128).

FIG. 44 shows the sequence of an exemplary ttGGBP181C Cysteine Scan Mutant (Table 3) expression construct (SEQ ID NO: 129).

FIG. 45 shows the sequence of an exemplary ttGGBP182C Cysteine Scan Mutant (Table 3) expression construct (SEQ ID NO: 130).

FIG. 46 shows the sequence of an exemplary ttGGBP183C Cysteine Scan Mutant (Table 3) expression construct (SEQ ID NO: 131).

FIG. 47 shows the sequence of an exemplary ttGGBP257C Cysteine Scan Mutant (Table 3) expression construct (SEQ ID NO: 132).

FIG. 48 shows the sequence of an exemplary ttGGBP259C Cysteine Scan Mutant (Table 3) expression construct (SEQ ID NO: 133).

FIG. 49 shows the sequence of an exemplary ttGGBP300C Cysteine Scan Mutant (Table 3) expression construct (SEQ ID NO: 134).

FIG. 50 shows the sequence of an exemplary ttGGBP17C.1 Affinity-Tuning Mutant (17C background) (Table 6) expression construct (SEQ ID NO: 135).

FIG. 51 shows the sequence of an exemplary ttGGBP17C.2 Affinity-Tuning Mutant (17C background) (Table 6) expression construct (SEQ ID NO: 136).

FIG. 52 shows the sequence of an exemplary ttGGBP17C.3 Affinity-Tuning Mutant (17C background) (Table 6) expression construct (SEQ ID NO: 137).

FIG. 53 shows the sequence of an exemplary ttGGBP17C.4 Affinity-Tuning Mutant (17C background) (Table 6) expression construct (SEQ ID NO: 138).

FIG. 54 shows the sequence of an exemplary ttGGBP17C.5 Affinity-Tuning Mutant (17C background) (Table 6) expression construct (SEQ ID NO: 139).

FIG. 55 shows the sequence of an exemplary ttGGBP17C.6 Affinity-Tuning Mutant (17C background) (Table 6) expression construct (SEQ ID NO: 140).

FIG. 56 shows the sequence of an exemplary ttGGBP17C.7 Affinity-Tuning Mutant (17C background) (Table 6) expression construct (SEQ ID NO: 141).

FIG. 57 shows the sequence of an exemplary ttGGBP17C.8 Affinity-Tuning Mutant (17C background) (Table 6) expression construct (SEQ ID NO: 142).

FIG. 58 shows the sequence of an exemplary ttGGBP17C.9 Affinity-Tuning Mutant (17C background) (Table 6) expression construct (SEQ ID NO: 143).

FIG. 59 shows the sequence of an exemplary ttGGBP17C.10 Affinity-Tuning Mutant (17C background) (Table 6) expression construct (SEQ ID NO: 144).

FIG. 60 shows the sequence of an exemplary ttGGBP17C.11 Affinity-Tuning Mutant (17C background) (Table 6) expression construct (SEQ ID NO: 145).

FIG. 61 shows the sequence of an exemplary ttGGBP17C.19 Affinity-Tuning Mutant (17C background) (Table 6) expression construct (SEQ ID NO: 146).

FIG. 62 shows the sequence of an exemplary ttGGBP17C.20 Affinity-Tuning Mutant (17C background) (Table 6) expression construct (SEQ ID NO: 147).

FIG. 63 shows the sequence of an exemplary ttGGBP17C.21 Affinity-Tuning Mutant (17C background) (Table 6) expression construct (SEQ ID NO: 148).

FIG. 64 shows the sequence of an exemplary ttGGBP17C.22 Affinity-Tuning Mutant (17C background) (Table 6) expression construct (SEQ ID NO: 149).

FIG. 65 shows the sequence of an exemplary ttGGBP17C.23 Affinity-Tuning Mutant (17C background) (Table 6) expression construct (SEQ ID NO: 150).

FIG. 66 shows the sequence of an exemplary ttGGBP17C.24 Affinity-Tuning Mutant (17C background) (Table 6) expression construct (SEQ ID NO: 151).

FIG. 67 shows the sequence of an exemplary ttGGBP17C.25 Affinity-Tuning Mutant (17C background) (Table 6) expression construct (SEQ ID NO: 152).

FIG. 68 shows the sequence of an exemplary ttGGBP17C.26 Affinity-Tuning Mutant (17C background) (Table 6) expression construct (SEQ ID NO: 153).

FIG. 69 shows the sequence of an exemplary ttGGBP17C.27 Affinity-Tuning Mutant (17C background) (Table 6) expression construct (SEQ ID NO: 154).

FIG. 70 shows the sequence of an exemplary ttGGBP17C.28 Affinity-Tuning Mutant (17C background) (Table 6) expression construct (SEQ ID NO: 155).

FIG. 71 shows the sequence of an exemplary ttGGBP17C.29 Affinity-Tuning Mutant (17C background) (Table 6) expression construct (SEQ ID NO: 156).

FIG. 72 shows the sequence of an exemplary ttGGBP182C.2.0 Affinity-Tuning Mutant (182C background) (Table 6) expression construct (SEQ ID NO: 157).

FIG. 73 shows the sequence of an exemplary ttGGBP182C.2.1 Affinity-Tuning Mutant (182C background) (Table 6) expression construct (SEQ ID NO: 158).

FIG. 74 shows the sequence of an exemplary ttGGBP182C.2.3 Affinity-Tuning Mutant (182C background) (Table 6) expression construct (SEQ ID NO: 159).

FIG. 75 shows the sequence of an exemplary ttGGBP182C.2.4 Affinity-Tuning Mutant (182C background) (Table 6) expression construct (SEQ ID NO: 160).

FIG. 76 shows the sequence of an exemplary ttGGBP182C.2.5 Affinity-Tuning Mutant (182C background) (Table 6) expression construct (SEQ ID NO: 161).

FIG. 77 shows the sequence of an exemplary ttGGBP182C.2.6 Affinity-Tuning Mutant (182C background) (Table 6) expression construct (SEQ ID NO: 162).

FIG. 78 shows the sequence of an exemplary ttGGBP182C.2.7 Affinity-Tuning Mutant (182C background) (Table 6) expression construct (SEQ ID NO: 163).

FIG. 79 shows the sequence of an exemplary ttGGBP182C.2.8 Affinity-Tuning Mutant (182C background) (Table 6) expression construct (SEQ ID NO: 164).

FIG. 80 shows the sequence of an exemplary ttGGBP182C.2.9 Affinity-Tuning Mutant (182C background) (Table 6) expression construct (SEQ ID NO: 165).

FIG. 81 shows the sequence of an exemplary ttGGBP182C.3 Affinity-Tuning Mutant (182C background) (Table 6) expression construct (SEQ ID NO: 166).

FIG. 82 shows the sequence of an exemplary ttGGBP182C.4 Affinity-Tuning Mutant (182C background) (Table 6) expression construct (SEQ ID NO: 167).

FIG. 83 shows the sequence of an exemplary ttGGBP182C.5 Affinity-Tuning Mutant (182C background) (Table 6) expression construct (SEQ ID NO: 168).

FIG. 84 shows the sequence of an exemplary ttGGBP182C.6 Affinity-Tuning Mutant (182C background) (Table 6) expression construct (SEQ ID NO: 169).

FIG. 85 shows the sequence of an exemplary ttGGBP182C.7 Affinity-Tuning Mutant (182C background) (Table 6) expression construct (SEQ ID NO: 170).

FIG. 86 shows the sequence of an exemplary ttGGBP182C.8 Affinity-Tuning Mutant (182C background) (Table 6) expression construct (SEQ ID NO: 171).

FIG. 87 shows the sequence of an exemplary ttGGBP182C.9 Affinity-Tuning Mutant (182C background) (Table 6) expression construct (SEQ ID NO: 172).

FIG. 88 is a cartoon illustrating non-limiting aspects of continuous glucose monitoring.

FIG. 89 is a cartoon illustrating a non-limiting example of sample analysis with a biosensor.

### DETAILED DESCRIPTION

Fluorescently responsive sensors (FRSs) based on engineered proteins that couple ligand-binding events to changes in the emission properties of intrinsic or semi-synthetically incorporated chromophores have wide-ranging applications in cell biology and analytical chemistry. If the fluorescence emission spectrum of an engineered FRS changes shape in response to ligand binding such that the ratio of intensities at two appropriately chosen wavelengths reports on ligand concentration (dichromatic response), then ratiometric measurements can be used to monitor analyte concentrations. Ratiometry Advantage is essential for devices that rely on changes in fluorescence emission intensities, because it provides an internally consistent reference. The self-calibrating nature of a ratiometric measurement removes the necessity for carrying out on-board calibration tests prior to each measurement, obviating the need for multiple components and fluidic circuitry. Accordingly, reagentless, ratiometric fluorescent sensors have many uses in process engineering, environmental or clinical chemistry, including single-use point-of-care applications, wearable devices, or implanted “tattoos” that are interrogated transdermally.

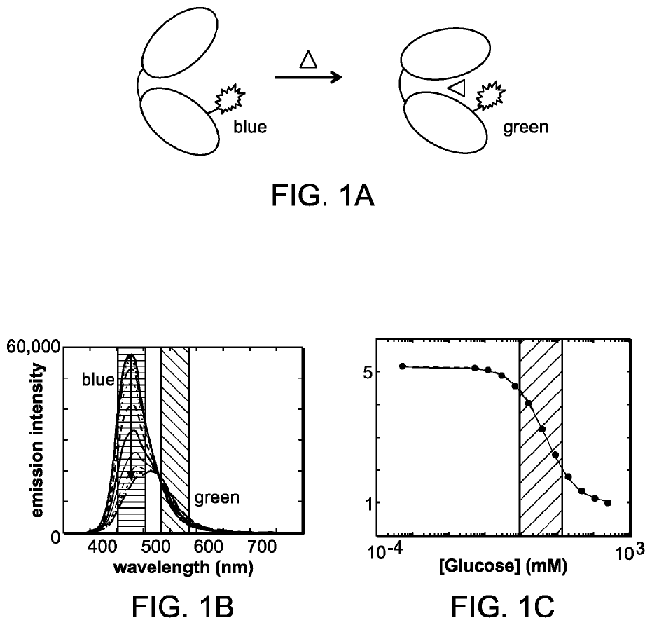

The periplasmic binding protein (PBP) superfamily provide a rich source of FRSs, because PBPs combine a large diversity of ligand specificities with a common structural mechanism that is well suited to the construction of fluorescence signal transduction schemes. The three-dimensional PBP monomer structure comprises two α/β domains linked by a β-strand hinge FIG. 1A. Binding of ligand is accompanied by a large hinge-bending motion that transitions the protein from an open to a closed state in which the ligand is enveloped within a cleft between the two domains. Semi-synthetic FRSs can be engineered with PBPs by site-specifically attaching single, thiol-reactive, environmentally sensitive fluorophores that respond to the ligand-mediated conformational change (FIGS. 1A-C). Here we describe the construction of robust, ratiometric, fluorescently responsive glucose sensors derived from thermostable members of a family of glucose-binding PBPs.

Glucose monitoring is essential for the management of diabetes mellitus, a disease that affects at least 366 million people world-wide, increasing every year. The majority of current glucose-monitoring technologies rely on enzymes for which glucose is one of the substrates. Glucose concentration measurements are therefore subject to variations in second substrate concentrations consumed in the enzyme reaction, such as oxygen in the case of glucose oxidase. Additional complications arise in systems where reaction rates are measured for enzymes immobilized on electrodes. In such arrangements, accuracy is compromised by factors that alter the rate at which glucose arrives at the electrode surface interfere with accuracy, such as hematocrit levels, or surface “fouling” by deposition of proteins and cells in the foreign body response. Ratiometric fluorescent glucose sensors obviate these problems, and accordingly have been incorporated successfully in optodes for continuous glucose monitoring in animals and humans.

In FRS-based sensors, signals arise from reversible binding equilibria of the analyte (ligand) to a receptor. These signals are most precise at ligand concentrations that match the receptor ligand-disassociation constant. Precision is maintained to within ˜80% of this maximal level over a concentration range approximately 3-fold above or below this point. Construction of effective FRS therefore requires matching of ligand-binding affinities to the relevant analyte concentrations. Arrays of multiple sensors may have to be used in concert to cover wide concentration ranges. Clinically relevant glucose levels vary approximately 100-fold (from ˜1 mM in extreme hypoglycemia, to ˜100 mM for the hyperosmolar, hyperglycemic condition, with healthy, euglycemic levels at ˜6 mM (Association 2000 Clinical Diabetes, 18; Pasquel 2014 Diabetes Care, 37, 3124-3131), requiring an array of multiple FRS sensors with distinct glucose affinities to report directly on the full range of clinically relevant glucose concentrations with high precision. Here we report a set of appropriately tuned thermostable, glucose-responsive FRSs, constructed by mutating their glucose-binding site.

Immobilization of FRSs on solid surfaces with minimal perturbation of the molecular sensing mechanism is an important step for incorporating biosensors into devices. Immobilization enables retention of the sensor within the sampling element (e.g. optode surface or implanted bead for in vivo sensing applications; or in a sample-handling cartridge for ex vivo sensing). Immobilization also may provide spatial localization to provide the necessary addressability of different elements in a multi-sensor array comprising sensors that differ in their engineered affinities for coverage of a wide range of glucose concentrations, or sensors that each detect distinct analytes.

Ex vivo clinical chemistries such as point-of-care applications require that the FRS is incorporated into a cartridge into which a sample is introduced at the time of measurement. Such “disposables” need to have a long shelf life that preferably does not require temperature control (e.g. refrigeration) for storage or distribution. It is preferable to incorporate immobilized protein in a stable, dried form in such disposables. The inherent resistance to denaturation of thermostable proteins minimizes the need for temperature control during manufacturing and storage, and may extend to the long-term stability of a desiccated state. The protein sensors described herein meet these requirements.

The spectral response, binding affinity, and thermostability of the robust thermostable glucose FRSs reported here are maintained following site-specific immobilization on a substrate such as beads. Furthermore, these properties are recovered rapidly upon reconstitution following drying and prolonged storage under accelerated aging conditions. These engineered proteins therefore are useful for high-precision, wide-dynamic range glucose sensing applications, including continuous monitoring, point-of-care, wearable sensor systems.

### Biosensors

Biosensors are molecular recognition elements that transduce ligand-binding events into physical signals. Biosensors as detailed herein bind at least one ligand and emit a signal. A ligand-bound biosensor results in a signal that is different from the unbound biosensor. This difference facilitates detection of the at least one ligand and/or determination of ligand concentration. The biosensors may be used without the assistance of other reagents.

Described herein are novel engineered biosensors. These biosensors may have altered ligand-binding affinities, tailored ligand-binding specificities, and/or temperature dependencies of ligand binding or stability. For example, the herein described engineered glucose and galactose biosensors provide high-accuracy information related to extended glucose concentration ranges.

Binding of ligand mediates conformational changes in the biosensor, such as hinge-bending motions of the polypeptide. The conformational changes affect the environment of the reporter such that a change in the reporter-generated signal occurs. That is, without ligand bound, the biosensor results in signal generated from the reporter, and when ligand is bound, the signal generated from the reporter changes. The ligand-bound biosensor results in a reporter-generated signal that is different from the unbound biosensor.

In some embodiments, the methods and compositions include a plurality of a single type of biosensor. The biosensors may be identical in structure and function. For example, the biosensors of a single type may have the same polypeptide, the same reporter, and the same ligand affinity.

In other embodiments, the methods and compositions include a plurality of different types of biosensors. A plurality of these different types of biosensors may be arranged or incorporated in a panel. As used herein, a “panel” refers to two or more biosensors. The two or more biosensors may be different from each other. The biosensors may differ in structure and/or function. Biosensors may differ in polypeptide sequence, reporter, ligand affinities, or a combination thereof. Accordingly, there may be different types of biosensors. In some embodiments, each biosensor in the panel comprises the same reporter group. In some embodiments, each biosensor in the panel comprises a different reporter group. The panel may include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, or at least 100 biosensors.

The panel of biosensors includes at least one sensor element. “Sensor element” refers to a single spot, site, location, or well for the at least one biosensor, to which a sample or aliquot thereof may be applied. The panel may be a composite sensor or an array.

In some embodiments, the panel is a composite sensor. In a composite sensor, each sensor element includes a mixture of two or more different biosensors. In some embodiments, the composite sensor includes one sensor element. In some embodiments, the composite sensor includes two or more sensor elements. In some embodiments, signals are measured from a composite sensor in which the signals arise from one or more biosensors in the sensor element. For example, signals may be measured from a composite sensor in which the signals arise from a subset of the total number of biosensors in the sensor element. For example, signals may be measured from a composite sensor in which the signals arise from two of five biosensors in the sensor element.

In some embodiments, the panel is an array. In an array, each sensor element includes a single type of biosensor. An array comprises a plurality of individually and spatially localized sensor elements. Each sensor element includes a biosensor that is different than or the same as the biosensor of a different sensor element. In some embodiments, signals are measured from an array in which the signals arise separately from two or more selected biosensors in separate sensor elements. An array may comprise a plurality of sensor elements of a variety of sizes and configurations. An array may comprise a plurality of sensor elements arranged linearly. For example, an array may comprise a plurality of micrometer-sized sensor elements arranged in a single row. An array may comprise a plurality of sensor elements arranged in a grid. The grid may be two- or three-dimensional. In some embodiments, the grid is a spatially addressable grid. In some embodiments, the biosensors are incorporated into an array, such as a multichannel or multiplexed array.

The biosensors of the present disclosure can be used in any setting where glucose detection is required or desired, such a medical setting (e.g., determining the level of blood glucose in a subject), environmental setting (e.g., determining the level of glucose in an environmental sample), biological setting (e.g., determining the presence or amount of glucose in a reaction), or in process engineering, such as monitoring the amount of glucose in a fermentation reaction (e.g., beer/wine production, etc.). Other examples include, but are not limited to, uses in the food industry (Suleiman et al, In: Biosensor Design and Application: Mathewson and Finley Eds; American Chemical Society, Washington, DC 1992, vol. 511); in clinical chemistry (Wilkins et al., Med. Eng. Phys. 1996, 18, 273-288; Pickup, Tr. Biotech. 1993, 11, 285-291; Meyerhoff et al., Endricon 1966, 6, 51-58; Riklin et al., Nature 1995, 376, 672-675); Willner et al., J. Am. Chem. Soc. 1996, 118, 10321-10322); as the basis for the construction of a fluorescent flow cell containing immobilized GGBP-FAST conjugates (see, e.g., Wilkins et al., Med. Eng. Phys. 1966, 18, 273-288; Pickup, Tr. Biotech. 1993, 11, 285-291; Meyerhoff et al., Endricon. 1966, 6, 51; Group, New Engl. J. Med. 1993, 329, 977-986; Gough et al., Diabetes 1995, 44, 1005-1009); and in an implantable devices, such as those suitable for use as an artificial pancreas.

The biosensors as detailed herein may be administered in a variety of ways known by those of skill in the art, as appropriate for each application. Biosensors may be provided in a solution. The solution may be buffered. Biosensors may be provided in a solution and mixed directly with a sample. In some embodiments, a biosensor is immobilized onto a surface. Biosensors may be immobilized within a disposable cartridge into which a sample may be introduced or applied. Biosensors may be implanted or incorporated in a wearable device. The biosensor may be provided as an optode.

The biosensor may be attached to or incorporated in a wearable device. Wearable devices may include, for example, adhesive strips, patches, and contact lenses. The biosensor may be configured for placement in contact with a subject's skin or mucosal surface. In some embodiments, the biosensor is configured as an adhesive strip. In some embodiments, the biosensor is configured within or on the surface of a contact lens. In some embodiments, the contact lens is formed from a transparent substrate shaped to be worn directly over a subject's eye, as described in, for example, U.S. Pat. No. 8,608,310.

The biosensor may be implanted. The biosensor may be implanted in a subject's body. The biosensor may be implanted in a subject's blood vessel, vein, eye, natural or artificial pancreas, skin, or anywhere in the alimentary canal including the stomach, intestine and esophagus. The biosensor may be implanted in a subject with a microbead. In some embodiments, the biosensor is configured to be implanted in the skin. The biosensor may be implanted in a subject sub-dermally. The biosensor may generate the signal trans-dermally. In some embodiments, the biosensor may be implanted in a subject with transdermal microbeads, wherein the optical signals can be transmitted remotely between the biosensor and detecting device.

In some embodiments, the biosensor is administered as an optode. As used herein, “optode” refers to an optical fiber with a single biosensor, or a composite biosensor, immobilized at the surface or at the end. An “optode” may also be referred to as an “optrode.” In some embodiments, the biosensor is implanted in a subject as an optode. The optode may be incorporated with or into a needle. The optode may be incorporated with a probe such as endoscopy or colonoscopy probes. The optode may be used in a tumor, near a tumor, or at the periphery of a tumor. In some embodiments, the biosensor may be implanted in a subject as an optode, wherein the optical signals can be transmitted between the biosensor and detecting device using physical links. In some embodiments, the biosensor is administered as an optode to a sample or reaction. The optode may be contacted with a sample or reaction. In some embodiments, an optode is used to continuously or episodically monitor a ligand in a sample or reaction.

### Methods of Detecting the Presence of a Ligand

Provided herein is a method of detecting the presence of a ligand in a sample. The method may include contacting the biosensor with the sample; measuring a signal from the biosensor; and comparing the signal to a ligand-free control. A difference in signal indicates the presence of ligand in the sample.

Also provided herein is a method of detecting the presence of glucose in a sample. The method may include (a) providing a glucose biosensor disclosed herein in which the reporter group is attached the GGBP so that a signal transduced by the reporter group when the GGBP is bound to glucose differs from a signal transduced by the reporter group when the GGBP is not bound to glucose; (b) contacting the biosensor with the test sample under conditions such that the biosensor can bind to glucose present in the test sample; and (c) comparing the signal transduced by the reporter group when the biosensor is contacted with the test sample with the signal transduced by the reporter group when the biosensor is contacted with a glucose-free control sample, wherein a difference in the signal transduced by the reporter group when the biosensor is contacted with the test sample, as compared to when the biosensor is contacted with the control sample, indicates that the test sample contains glucose.

### Methods of Determining the Concentration of a Ligand

Provided herein is a method of determining the concentration of a ligand in a sample. The method may include contacting the biosensor with the sample; measuring a signal from the biosensor; and comparing the signal to a standard hyperbolic ligand binding curve to determine the concentration of ligand in the test sample. The standard hyperbolic ligand binding curve may be prepared by measuring the signal transduced by the biosensor when contacted with control samples containing known concentrations of ligand.

Another aspect of the present disclosure provides a method of determining the concentration of glucose in a test sample comprising, consisting of, or consisting essentially of: (a) providing a glucose biosensor comprising a glucose biosensor as described herein in which the reporter group is attached the GGBP so that a signal transduced by the reporter group when the GGBP is bound to glucose differs from a signal transduced by the reporter group when the GGBP is not bound to glucose; (b) contacting the biosensor with the test sample under conditions such that the biosensor can bind to glucose present in the test sample; and (c) comparing the signal transduced by the reporter group when the biosensor is contacted with the test sample with a standard hyperbolic glucose binding curve prepared by measuring the signal transduced by the reporter group when the biosensor is contacted with control samples containing known quantities of glucose to determine the concentration of glucose in the test sample.

### Methods of Monitoring the Presence of a Ligand

The present invention is directed to a method of episodically or continuously monitoring the presence of a ligand in a reaction. In certain embodiments, the biosensors may be used in the continuous monitoring of glucose in a reaction. In certain embodiments, the glucose sensors may be used in episodic monitoring of sample aliquots.

The method of episodically or continuously monitoring the presence of a ligand in a reaction may include contacting the biosensor with the reaction; maintaining the reaction under conditions such that the polypeptide is capable of binding ligand present in the reaction; and episodically or continuously monitoring the signal from the biosensor in the reaction.

The method of episodically or continuously monitoring the presence of a ligand in a reaction may include contacting the biosensor with the reaction; maintaining the reaction under conditions such that the polypeptide is capable of binding ligand present in the reaction; episodically or continuously monitoring the signal from the biosensor in the reaction; and comparing the signal to a standard hyperbolic ligand binding curve to determine the concentration of ligand in the test sample. The standard hyperbolic ligand binding curve may be prepared by measuring the signal transduced by the biosensor when contacted with control samples containing known concentrations of ligand.

In some embodiments, the method further includes comparing the signal to a ligand-free control, wherein a difference in signal indicates the presence of ligand in the reaction.

In some embodiments, the method further includes comparing the signal to a standard hyperbolic ligand binding curve to determine the concentration of ligand in the test sample. The standard hyperbolic ligand binding curve may be prepared by measuring the signal transduced by the biosensor when contacted with control samples containing known concentrations of ligand.

Another aspect of the present disclosure provides a method of continuously monitoring the presence of glucose in a reaction comprising, consisting of, or consisting essentially of: (a) providing a glucose biosensor as described herein in which the reporter group is attached the GGBP so that a signal transduced by the reporter group when the GGBP is bound to glucose differs from a signal transduced by the reporter group when the GGBP is not bound to glucose; (b) maintaining the biosensor within the reaction and under conditions such that the biosensor can bind to glucose present in the reaction; (c) continuously monitoring the signal transduced by the reporter group when the biosensor is contacted with the glucose present in the reaction; and optionally (d) comparing the signal transduced by the reporter group when the biosensor is contacted with the glucose present in the reaction with the signal transduced by the reporter group when the biosensor is contacted with a glucose-free control sample, wherein a difference in the signal transduced by the reporter group when the biosensor is contacted with the glucose present in the reaction, as compared to when the biosensor is contacted with the control sample, indicates glucose is present in the reaction.

Yet another aspect of the present disclosure provides a method of continuously monitoring the concentration of glucose in a reaction comprising, consisting of, or consisting essentially of: (a) providing a glucose biosensor comprising a glucose biosensor as described herein in which the reporter group is attached the GGBP so that a signal transduced by the reporter group when the GGBP is bound to glucose differs from a signal transduced by the reporter group when the GGBP is not bound to glucose; (b) maintaining the biosensor within the reaction under conditions such that the biosensor can bind to glucose present in the reaction; and (c) continuously monitoring the signal transduced by the reporter group when the biosensor is contacted with the glucose present in the reaction; and (d) comparing the signal transduced by the reporter group when the biosensor is contacted with the glucose present in the reaction with a standard hyperbolic glucose binding curve prepared by measuring the signal transduced by the reporter group when the biosensor is contacted with control samples containing known quantities of glucose to determine the concentration of glucose in the reaction.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes/nucleic acids or sequences/amino acids that flank it in its naturally-occurring state. Purified also defines a degree of sterility that is safe for administration to a human subject, e.g., lacking infectious or toxic agents. In the case of tumor antigens, the antigen may be purified or a processed preparation such as a tumor cell lysate.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

“Subject” as used herein refers to any organism from which a biological sample is obtained. For example, the sample is a biological fluid or tissue. For example, a subject is one who wants or is in need of detecting ligand or determining the concentration of ligand with the herein described biosensors. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

As used herein, an “expression vector” is a DNA or RNA vector that is capable of effecting expression of one or more polynucleotides. Preferably, the expression vector is also capable of replicating within the host cell. Expression vectors can be either prokaryotic or eukaryotic, and are typically include plasmids. Expression vectors of the present invention include any vectors that function (i.e., direct gene expression) in host cells of the present invention, including in one of the prokaryotic or eukaryotic cells described herein, e.g., gram-positive, gram-negative, pathogenic, non-pathogenic, commensal, cocci, bacillus, or spiral-shaped bacterial cells; archaeal cells; or protozoan, algal, fungi, yeast, plant, animal, vertebrate, invertebrate, arthropod, mammalian, rodent, primate, or human cells. Expression vectors of the present invention contain regulatory sequences such as transcription control sequences, translation control sequences, origins of replication, and other regulatory sequences that are compatible with the host cell and that control the expression of a polynucleotide. In particular, expression vectors of the present invention include transcription control sequences. Transcription control sequences are sequences which control the initiation, elongation, and termination of transcription. Particularly important transcription control sequences are those which control transcription initiation such as promoter, enhancer, operator and repressor sequences. Suitable transcription control sequences include any transcription control sequence that can function in at least one of the cells of the present invention. A variety of such transcription control sequences are known to those skilled in the art.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

The term “diagnosis” refers to a determination that a disease is present in the subject. Similarly, the term “prognosis” refers to a relative probability that a certain future outcome may occur in the subject. For example, in the context of the present disclosure, prognosis can refer to the likelihood that an individual will develop a disease, or the likely severity of the disease (e.g., severity of symptoms, rate of functional decline, survival, etc.).

Depending on context, the terms “polypeptide” and “protein” may be used interchangeably.

A polypeptide or class of polypeptides may be defined by the extent of identity (% identity) of its amino acid sequence to a reference amino acid sequence, or by having a greater % identity to one reference amino acid sequence than to another. A variant of any of genes or gene products disclosed herein may have, e.g., 50%, 55%, 60%, 65%, 70%, 75%, 80%, 85%, 90%, 95%, 96%, 97%, 98%, or 99% sequence identity to the nucleic acid or amino acid sequences described herein. The term “% identity,” in the context of two or more nucleic acid or polypeptide sequences, refers to two or more sequences or subsequences that are the same or have a specified percentage of amino acid residues or nucleotides that are the same, when compared and aligned for maximum correspondence, as measured using a sequence comparison algorithm or by visual inspection. For example, % identity is relative to the entire length of the coding regions of the sequences being compared, or the length of a particular fragment or functional domain thereof. Variants as disclosed herein also include homologs, orthologs, or paralogs of the genes or gene products described herein. In some embodiments, variants may demonstrate a percentage of homology or identity, for example, at least about 50%, 55%, 60%, 65%, 70%, 75%, 80%, 85%, 90%, 95%, 96%, 97%, 98%, or 99% identity conserved domains important for biological function, e.g., in a functional domain, e.g. a ligand-binding or catalytic domain.

For sequence comparison, one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity is determined using BLAST. For the BLAST searches, the following parameters were employed: (1) Expect threshold is 10; (2) Gap cost is Existence: 11 and Extension: 1; (3) The Matrix employed is BLOSUM62; (4) The filter for low complexity regions is “on.”

The present invention also provides for functional fragments of the genes or gene products described herein. A fragment of a protein is characterized by a length (number of amino acids) that is less than the length of the full length mature form of the protein. A fragment, in the case of these sequences and all others provided herein, may be a part of the whole that is less than the whole. Moreover, a fragment ranges in size from a single nucleotide or amino acid within a polynucleotide or polypeptide sequence to one fewer nucleotide or amino acid than the entire polynucleotide or polypeptide sequence. Finally, a fragment is defined as any portion of a complete polynucleotide or polypeptide sequence that is intermediate between the extremes defined above.

For example, fragments of any of the proteins or enzymes disclosed herein or encoded by any of the genes disclosed herein can be 10 to 20 amino acids, 10 to 30 amino acids, 10 to 40 amino acids, 10 to 50 amino acids, 10 to 60 amino acids, 10 to 70 amino acids, 10 to 80 amino acids, 10 to 90 amino acids, 10 to 100 amino acids, 50 to 100 amino acids, 75 to 125 amino acids, 100 to 150 amino acids, 150 to 200 amino acids, 200 to 250 amino acids, 250 to 300 amino acids, or 300 to 350 amino acids. The fragments encompassed in the present subject matter comprise fragments that retain functional fragments. As such, the fragments preferably retain the binding domains that are required or are important for functional activity. Fragments can be determined or generated by using the sequence information herein, and the fragments can be tested for functional activity using standard methods known in the art. For example, the encoded protein can be expressed by any recombinant technology known in the art and the binding activity of the protein can be determined.

As used herein a “biologically active” fragment is a portion of a polypeptide which maintains an activity of a full-length reference polypeptide. Biologically active fragments as used herein exclude the full-length polypeptide. Biologically active fragments can be any size as long as they maintain the defined activity. Preferably, the biologically active fragment maintains at least 10%, at least 50%, at least 75% or at least 90%, of the activity of the full length protein.

Amino acid sequence variants/mutants of the polypeptides of the defined herein can be prepared by introducing appropriate nucleotide changes into a nucleic acid defined herein, or by in vitro synthesis of the desired polypeptide. Such variants/mutants include, for example, deletions, insertions or substitutions of residues within the amino acid sequence. A combination of deletion, insertion and substitution can be made to arrive at the final construct, provided that the final peptide product possesses the desired activity and/or specificity.

Mutant (altered) peptides can be prepared using any technique known in the art. For example, a polynucleotide defined herein can be subjected to in vitro mutagenesis or DNA shuffling techniques as broadly described by Harayama (1998). Products derived from mutated/altered DNA can readily be screened using techniques described herein to determine if they possess, for example, glucose and/or galactose binding activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues. In some embodiments, a mutated or modified protein does not comprise any deletions or insertions. In various embodiments, a mutated or modified protein has less than about 10, 9, 8, 7, 6, 5, 4, 3, or 2 deleted or inserted amino acids.

Substitution mutants have at least one amino acid residue in the polypeptide molecule removed and a different residue inserted in its place. Sites may be substituted in a relatively conservative manner in order to maintain activity and/or specificity. Such conservative substitutions are shown in the table below under the heading of “exemplary substitutions.”

In certain embodiments, a mutant/variant polypeptide has only, or not more than, one or two or three or four conservative amino acid changes when compared to a naturally occurring polypeptide. Details of conservative amino acid changes are provided in the table below. As the skilled person would be aware, such minor changes can reasonably be predicted not to alter the activity of the polypeptide when expressed in a recombinant cell.

### Exemplary Substitutions

| Original Residue | Exemplary Substitutions |
|---|---|
| Alanine (Ala) | Val; Leu; Ile; Gly |
| Arginine (Arg) | Lys |
| Asparagine (Asn) | Gln; His |
| Cysteine (Cys) | Ser |
| Glutamine (Gln) | Asn; His |
| Glutamic Acid (Glu) | Asp |
| Glycine (Gly) | Pro; Ala |
| Histidine (His) | Asn; Gln |
| Isoleucine (Ile) | Leu; Val; Ala |
| Leucine (Leu) | Ile; Val; Met; Ala; Phe |
| Lysine (Lys) | Arg |
| Methionine (Met) | Leu; Phe |
| Phenylalanine (Phe) | Leu; Val; Ala |
| Proline (Pro) | Gly |
| Serine (Ser) | Thr |
| Threonine (Thr) | Ser |
| Tryptophan (Trp) | Tyr |
| Tyrosine (Tyr) | Trp; Phe |
| Valine (Val) | Ile; Leu; Met; Phe; Ala |

Mutations can be introduced into a nucleic acid sequence such that the encoded amino acid sequence is altered by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Certain amino acids have side chains with more than one classifiable characteristic. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, tryptophan, cysteine), nonpolar side chains (e.g., alanine, valine, leucine, isoleucine, proline, phenylalanine, methionine, tyrosine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a given polypeptide is replaced with another amino acid residue from the same side chain family. Alternatively, in another embodiment, mutations can be introduced randomly along all or part of a given coding sequence, such as by saturation mutagenesis, and the resultant mutants can be screened for given polypeptide biological activity to identify mutants that retain activity. Conversely, the invention also provides for variants with mutations that enhance or increase the endogenous biological activity. Following mutagenesis of the nucleic acid sequence, the encoded protein can be expressed by any recombinant technology known in the art and the activity/specificity of the protein can be determined. An increase, decrease, or elimination of a given biological activity of the variants disclosed herein can be readily measured by the ordinary person skilled in the art, i.e., by measuring the capability for binding a ligand and/or signal transduction.

In various embodiments, a polypeptide comprises mutations such that 1, 2, 3, 4, 5, 6, 7, 8, 9, or 10, or less than about 10, 9, 8, 7, 6, 5, 4, 3, or 2 amino acids is substituted with a cysteine and/or a lysine.

Polypeptides can be produced in a variety of ways, including production and recovery of natural polypeptides or recombinant polypeptides according to methods known in the art. In one embodiment, a recombinant polypeptide is produced by culturing a cell capable of expressing the polypeptide under conditions effective to produce the polypeptide, such as a host cell defined herein.

### Key to the Sequence Listing

| SEQ ID NO | Sequence Name |
|---|---|
| 1 | ecGGBP [U.S. National Center for Biotechnology Information (NCBI) |
| | Accession No. WP_032329053] |
| 2 | ttGGBP (NCBI Accession Nos. YP_003852930.1 and WP_013298803.1) |
| 3 | stGGBP (NCBI Accession No. WP_001036943) |
| 4 | chyGGBP (NCBI Accession Nos. WP_013402088.1 and |
| | YP_003991244.1) |
| 5 | cobGGBP (NCBI Accession Nos. WP_013289482.1 and |
| | YP_003839461.1) |
| 6 | pspGGBP (NCBI Accession Nos. WP_015735911.1 and |
| | YP_003243743.1) |
| 7 | csaGGBP (NCBI Accession Nos. WP_013273028.1 and |
| | YP_003822565.1) |
| 8 | bprGGBP (NCBI Accession Nos. WP_013280279.1 and |
| | YP_003830205.1) |
| 9 | rinGGBP_A (NCBI Accession Nos. WP_006855636.1 and |
| | YP_007778116.1) |
| 10 | fprGGBP (NCBI Accession Nos. WP_015536639.1 and |
| | YP_007799070.1) |
| 11 | cljGGBP (NCBI Accession No. CLJU_c08950) |
| 12 | cauGGBP (NCBI Accession No. CAETHG_2989) |
| 13 | rinGGBP_B (NCBI Accession Nos. WP_006855628.1 and |
| | YP_007778124.1) |
| 14 | erhGGBP (NCBI Accession Nos. WP_003775352.1 and |
| | YP_004561181.1) |
| 15 | ereGGBP (NCBI Accession Nos. WP_012741392.1 and |
| | YP_002936409.1) |
| 16 | ecGGBP (with signal peptide replaced with M) |
| 17 | ecGGBP (with signal sequence removed) |
| 18 | ttGGBP (with signal peptide replaced with M) |
| 19 | stGGBP (with signal peptide replaced with M) |
| 20 | chyGGBP (with signal peptide replaced with M) |
| 21 | cobGGBP (with signal peptide replaced with M) |
| 22 | pspGGBP (with signal peptide replaced with M) |
| 23 | csaGGBP (with signal peptide replaced with M) |
| 24 | bprGGBP (with signal peptide replaced with M) |
| 25 | rinGGBP_A (with signal peptide replaced with M) |
| 26 | fprGGBP (with signal peptide replaced with M) |
| 27 | cljGGBP (with signal peptide replaced with M) |
| 28 | cauGGBP (with signal peptide replaced with M) |
| 29 | rinGGBP_b (with signal peptide replaced with M) |
| 30 | erhGGBP (with signal peptide replaced with M) |
| 31 | ereGGBP (with signal peptide replaced with M) |
| 32 | ecGGBPW183C (with signal peptide replaced with M; a W183C |
| | mutation; and a GGSHHHHHH at C-terminus) |
| 33 | ecGGBP (with signal peptide replaced with M and a GGSHHHHHH at C- |
| | terminus) |
| 34 | csaGGBP (with signal peptide replaced with M and a GGSHHHHHH at |
| | C-terminus) |
| 35 | bprGGBP (with signal peptide replaced with M and a GGSHHHHHH at |
| | C-terminus) |
| 36 | rinGGBP_A (with signal peptide replaced with M and a GGSHHHHHH |
| | at C-terminus) |
| 37 | fprGGBP (with signal peptide replaced with M and a GGSHHHHHH at |
| | C-terminus) |
| 38 | cljGGBP (with signal peptide replaced with M and a GGSHHHHHH at C- |
| | terminus) |
| 39 | cauGGBP (with signal peptide replaced with M and a GGSHHHHHH at |
| | C-terminus) |
| 40 | rinGGBP_B (with signal peptide replaced with M and a GGSHHHHHH at |
| | C-terminus) |
| 41 | erhGGBP (with signal peptide replaced with M and a GGSHHHHHH at |
| | C-terminus) |
| 42 | ereGGBP (with signal peptide replaced with M and a GGSHHHHHH at |
| | C-terminus) |
| 43 | ttGGBP (with signal peptide replaced with M and a GGSHHHHHH at C- |
| | terminus) |
| 44 | cobGGBP (with signal peptide replaced with M and a GGSHHHHHH at |
| | C-terminus) |
| 45 | chyGGBP (with signal peptide replaced with M and a GGSHHHHHH at |
| | C-terminus) |
| 46 | pspGGBP (with signal peptide replaced with M and a GGSHHHHHH at |
| | C-terminus) |
| 47 | cobGGBP.W181C (Signaling cys mutant of cobGGBP with signal peptide |
| | replaced with M and GGSHHHHHH at C-terminus) |
| 48 | chyGGBP.W181C (Signaling cys mutants of chyGGBP with signal |
| | peptide replaced with M and GGSHHHHHH at C-terminus) |
| 49 | ttGGBP11C (ttGGBP cys mutant with signal peptide replaced with M and |
| | GGSHHHHHH at C-terminus) |
| 50 | ttGGBP16C (ttGGBP cys mutant with signal peptide replaced with M and |
| | GGSHHHHHH at C-terminus) |
| 51 | ttGGBP17C (ttGGBP cys mutant with signal peptide replaced with M and |
| | GGSHHHHHH at C-terminus) |
| 52 | ttGGBP42C (ttGGBP cys mutant with signal peptide replaced with M and |
| | GGSHHHHHH at C-terminus) |
| 53 | ttGGBP67C (ttGGBP cys mutant with signal peptide replaced with M and |
| | GGSHHHHHH at C-terminus) |
| 54 | ttGGBP91C (ttGGBP cys mutant with signal peptide replaced with M and |
| | GGSHHHHHH at C-terminus) |
| 55 | ttGGBP92C (ttGGBP cys mutant with signal peptide replaced with M and |
| | GGSHHHHHH at C-terminus) |
| 56 | ttGGBP111C (ttGGBP cys mutant with signal peptide replaced with M |
| | and GGSHHHHHH at C-terminus) |
| 57 | ttGGBP148C (ttGGBP cys mutant with signal peptide replaced with M |
| | and GGSHHHHHH at C-terminus) |
| 58 | ttGGBP151C (ttGGBP cys mutant with signal peptide replaced with M |
| | and GGSHHHHHH at C-terminus) |
| 59 | ttGGBP152C (ttGGBP cys mutant with signal peptide replaced with M |
| | and GGSHHHHHH at C-terminus) |
| 60 | ttGGBP181C (ttGGBP cys mutant with signal peptide replaced with M |
| | and GGSHHHHHH at C-terminus) |
| 61 | ttGGBP182C (ttGGBP cys mutant with signal peptide replaced with M |
| | and GGSHHHHHH at C-terminus) |
| 62 | ttGGBP183C (ttGGBP cys mutant with signal peptide replaced with M |
| | and GGSHHHHHH at C-terminus) |
| 63 | ttGGBP257C (ttGGBP cys mutant with signal peptide replaced with M |
| | and GGSHHHHHH at C-terminus) |
| 64 | ttGGBP259C (ttGGBP cys mutant with signal peptide replaced with M |
| | and GGSHHHHHH at C-terminus) |
| 65 | ttGGBP300C (ttGGBP cys mutant with signal peptide replaced with M |
| | and GGSHHHHHH at C-terminus) |
| 66 | ttGGBP17C.1 (affinity-tuning mutant, 17C background (Table 6) with |
| | signal peptide replaced with M and GGSHHHHHH at C-terminus) |
| 67 | ttGGBP17C.2 (affinity-tuning mutant, 17C background (Table 6) with |
| | signal peptide replaced with M and GGSHHHHHH at C-terminus) |
| 68 | ttGGBP17C.3 (affinity-tuning mutant, 17C background (Table 6) with |
| | signal peptide replaced with M and GGSHHHHHH at C-terminus) |
| 69 | ttGGBP17C.4 (affinity-tuning mutant, 17C background (Table 6) with |
| | signal peptide replaced with M and GGSHHHHHH at C-terminus) |
| 70 | ttGGBP17C.5 (affinity-tuning mutant, 17C background (Table 6) with |
| | signal peptide replaced with M and GGSHHHHHH at C-terminus) |
| 71 | ttGGBP17C.6 (affinity-tuning mutant, 17C background (Table 6) with |
| | signal peptide replaced with M and GGSHHHHHH at C-terminus) |
| 72 | ttGGBP17C.7 (affinity-tuning mutant, 17C background (Table 6) with |
| | signal peptide replaced with M and GGSHHHHHH at C-terminus) |
| 73 | ttGGBP17C.8 (affinity-tuning mutant, 17C background (Table 6) with |
| | signal peptide replaced with M and GGSHHHHHH at C-terminus) |
| 74 | ttGGBP17C.9 (affinity-tuning mutant, 17C background (Table 6) with |
| | signal peptide replaced with M and GGSHHHHHH at C-terminus) |
| 75 | ttGGBP17C.10 (affinity-tuning mutant, 17C background (Table 6) with |
| | signal peptide replaced with M and GGSHHHHHH at C-terminus) |
| 76 | ttGGBP17C.11 (affinity-tuning mutant, 17C background (Table 6) with |
| | signal peptide replaced with M and GGSHHHHHH at C-terminus) |
| 77 | ttGGBP17C.19 (affinity-tuning mutant, 17C background (Table 6) with |
| | signal peptide replaced with M and GGSHHHHHH at C-terminus) |
| 78 | ttGGBP17C.20 (affinity-tuning mutant, 17C background (Table 6) with |
| | signal peptide replaced with M and GGSHHHHHH at C-terminus) |
| 79 | ttGGBP17C.21 (affinity-tuning mutant, 17C background (Table 6) with |
| | signal peptide replaced with M and GGSHHHHHH at C-terminus) |
| 80 | ttGGBP17C.22 (affinity-tuning mutant, 17C background (Table 6) with |
| | signal peptide replaced with M and GGSHHHHHH at C-terminus) |
| 81 | ttGGBP17C.23 (affinity-tuning mutant, 17C background (Table 6) with |
| | signal peptide replaced with M and GGSHHHHHH at C-terminus) |
| 82 | ttGGBP17C.24 (affinity-tuning mutant, 17C background (Table 6) with |
| | signal peptide replaced with M and GGSHHHHHH at C-terminus) |
| 83 | ttGGBP17C.25 (affinity-tuning mutant, 17C background (Table 6) with |
| | signal peptide replaced with M and GGSHHHHHH at C-terminus) |
| 84 | ttGGBP17C.26 (affinity-tuning mutant, 17C background (Table 6) with |
| | signal peptide replaced with M and GGSHHHHHH at C-terminus) |
| 85 | ttGGBP17C.27 (affinity-tuning mutant, 17C background (Table 6) with |
| | signal peptide replaced with M and GGSHHHHHH at C-terminus) |
| 86 | ttGGBP17C.28 (affinity-tuning mutant, 17C background (Table 6) with |
| | signal peptide replaced with M and GGSHHHHHH at C-terminus) |
| 87 | ttGGBP17C.29 (affinity-tuning mutant, 17C background (Table 6) with |
| | signal peptide replaced with M and GGSHHHHHH at C-terminus) |
| 88 | ttGGBP182C.2.0 (affinity-tuning mutant, 182C background (Table 6) |
| | with signal peptide replaced with M and GGSHHHHHH at C-terminus) |
| 89 | ttGGBP182C.2.1 (affinity-tuning mutant, 182C background (Table 6) |
| | with signal peptide replaced with M and GGSHHHHHH at C-terminus) |
| 90 | ttGGBP182C.2.3 (affinity-tuning mutant, 182C background (Table 6) |
| | with signal peptide replaced with M and GGSHHHHHH at C-terminus) |
| 91 | ttGGBP182C.2.4 (affinity-tuning mutant, 182C background (Table 6) |
| | with signal peptide replaced with M and GGSHHHHHH at C-terminus) |
| 92 | ttGGBP182C.2.5 (affinity-tuning mutant, 182C background (Table 6) |
| | with signal peptide replaced with M and GGSHHHHHH at C-terminus) |
| 93 | ttGGBP182C.2.6 (affinity-tuning mutant, 182C background (Table 6) |
| | with signal peptide replaced with M and GGSHHHHHH at C-terminus) |
| 94 | ttGGBP182C.2.7 (affinity-tuning mutant, 182C background (Table 6) |
| | with signal peptide replaced with M and GGSHHHHHH at C-terminus) |
| 95 | ttGGBP182C.2.8 (affinity-tuning mutant, 182C background (Table 6) |
| | with signal peptide replaced with M and GGSHHHHHH at C-terminus) |
| 96 | ttGGBP182C.2.9 (affinity-tuning mutant, 182C background (Table 6) |
| | with signal peptide replaced with M and GGSHHHHHH at C-terminus) |
| 97 | ttGGBP182C.3 (affinity-tuning mutant, 182C background (Table 6) with |
| | signal peptide replaced with M and GGSHHHHHH at C-terminus) |
| 98 | ttGGBP182C.4 (affinity-tuning mutant, 182C background (Table 6) with |
| | signal peptide replaced with M and GGSHHHHHH at C-terminus) |
| 99 | ttGGBP182C.5 (affinity-tuning mutant, 182C background (Table 6) with |
| | signal peptide replaced with M and GGSHHHHHH at C-terminus) |
| 100 | ttGGBP182C.6 (affinity-tuning mutant, 182C background (Table 6) with |
| | signal peptide replaced with M and GGSHHHHHH at C-terminus) |
| 101 | ttGGBP182C.7 (affinity-tuning mutant, 182C background (Table 6) with |
| | signal peptide replaced with M and GGSHHHHHH at C-terminus) |
| 102 | ttGGBP182C.8 (affinity-tuning mutant, 182C background (Table 6) with |
| | signal peptide replaced with M and GGSHHHHHH at C-terminus) |
| 103 | ttGGBP182C.9 (affinity-tuning mutant, 182C background (Table 6) with |
| | signal peptide replaced with M and GGSHHHHHH at C-terminus) |
| 104 | GGSHHHHHH |
| 105 | csaGGBP Exemplary Expression Construct (Table 2) |
| 106 | bprGGBP Exemplary Expression Construct (Table 2) |
| 107 | rinGGBP_A Exemplary Expression Construct (Table 2) |
| 108 | fprGGBP Exemplary Expression Construct (Table 2) |
| 109 | cljGGBP Exemplary Expression Construct (Table 2) |
| 110 | cauGGBP Exemplary Expression Construct (Table 2) |
| 111 | rinGGBP_B Exemplary Expression Construct (Table 2) |
| 112 | erhGGBP Exemplary Expression Construct (Table 2) |
| 113 | ereGGBP Exemplary Expression Construct (Table 2) |
| 114 | ttGGBP Exemplary Expression Construct (Table 2) |
| 115 | cobGGBP Exemplary Expression Construct (Table 2) |
| 116 | chyGGBP Exemplary Expression Construct (Table 2) |
| 117 | pspGGBP23 Exemplary Expression Construct (Table 2) |
| 118 | ttGGBP11C Exemplary Cysteine Scan Mutant Expression Construct |
| | (Table 3) |
| 119 | ttGGBP16C Exemplary Cysteine Scan Mutant Expression Construct |
| | (Table 3) |
| 120 | ttGGBP17C Exemplary Cysteine Scan Mutant Expression Construct |
| | (Table 3) |
| 121 | ttGGBP42C Exemplary Cysteine Scan Mutant Expression Construct |
| | (Table 3) |
| 122 | ttGGBP67C Exemplary Cysteine Scan Mutant Expression Construct |
| | (Table 3) |
| 123 | ttGGBP91C Exemplary Cysteine Scan Mutant Expression Construct |
| | (Table 3) |
| 124 | ttGGBP92C Exemplary Cysteine Scan Mutant Expression Construct |
| | (Table 3) |
| 125 | ttGGBP111C Exemplary Cysteine Scan Mutant Expression Construct |
| | (Table 3) |
| 126 | ttGGBP148C Exemplary Cysteine Scan Mutant Expression Construct |
| | (Table 3) |
| 127 | ttGGBP151C Exemplary Cysteine Scan Mutant Expression Construct |
| | (Table 3) |
| 128 | ttGGBP152C Exemplary Cysteine Scan Mutant Expression Construct |
| | (Table 3) |
| 129 | ttGGBP181C Exemplary Cysteine Scan Mutant Expression Construct |
| | (Table 3) |
| 130 | ttGGBP182C Exemplary Cysteine Scan Mutant Expression Construct |
| | (Table 3) |
| 131 | ttGGBP183C Exemplary Cysteine Scan Mutant Expression Construct |
| | (Table 3) |
| 132 | ttGGBP257C Exemplary Cysteine Scan Mutant Expression Construct |
| | (Table 3) |
| 133 | ttGGBP259C Exemplary Cysteine Scan Mutant Expression Construct |
| | (Table 3) |
| 134 | ttGGBP300C Exemplary Cysteine Scan Mutant Expression Construct |
| | (Table 3) |
| 135 | ttGGBP17C.1 Exemplary Affinity-Tuning mutant (17C Background) |
| | Expression Construct (Table 6) |
| 136 | ttGGBP17C.2 Exemplary Affinity-Tuning mutant (17C Background) |
| | Expression Construct (Table 6) |
| 137 | ttGGBP17C.3 Exemplary Affinity-Tuning mutant (17C Background) |
| | Expression Construct (Table 6) |
| 138 | ttGGBP17C.4 Exemplary Affinity-Tuning mutant (17C Background) |
| | Expression Construct (Table 6) |
| 139 | ttGGBP17C.5 Exemplary Affinity-Tuning mutant (17C Background) |
| | Expression Construct (Table 6) |
| 140 | ttGGBP17C.6 Exemplary Affinity-Tuning mutant (17C Background) |
| | Expression Construct (Table 6) |
| 141 | ttGGBP17C.7 Exemplary Affinity-Tuning mutant (17C Background) |
| | Expression Construct (Table 6) |
| 142 | ttGGBP17C.8 Exemplary Affinity-Tuning mutant (17C Background) |
| | Expression Construct (Table 6) |
| 143 | ttGGBP17C.9 Exemplary Affinity-Tuning mutant (17C Background) |
| | Expression Construct (Table 6) |
| 144 | ttGGBP17C.10 Exemplary Affinity-Tuning mutant (17C Background) |
| | Expression Construct (Table 6) |
| 145 | ttGGBP17C.11 Exemplary Affinity-Tuning mutant (17C Background) |
| | Expression Construct (Table 6) |
| 146 | ttGGBP17C.19 Exemplary Affinity-Tuning mutant (17C Background) |
| | Expression Construct (Table 6) |
| 147 | ttGGBP17C.20 Exemplary Affinity-Tuning mutant (17C Background) |
| | Expression Construct (Table 6) |
| 148 | ttGGBP17C.21 Exemplary Affinity-Tuning mutant (17C Background) |
| | Expression Construct (Table 6) |
| 149 | ttGGBP17C.22 Exemplary Affinity-Tuning mutant (17C Background) |
| | Expression Construct (Table 6) |
| 150 | ttGGBP17C.23 Exemplary Affinity-Tuning mutant (17C Background) |
| | Expression Construct (Table 6) |
| 151 | ttGGBP17C.24 Exemplary Affinity-Tuning mutant (17C Background) |
| | Expression Construct (Table 6) |
| 152 | ttGGBP17C.25 Exemplary Affinity-Tuning mutant (17C Background) |
| | Expression Construct (Table 6) |
| 153 | ttGGBP17C.26 Exemplary Affinity-Tuning mutant (17C Background) |
| | Expression Construct (Table 6) |
| 154 | ttGGBP17C.27 Exemplary Affinity-Tuning mutant (17C Background) |
| | Expression Construct (Table 6) |
| 155 | ttGGBP17C.28 Exemplary Affinity-Tuning mutant (17C Background) |
| | Expression Construct (Table 6) |
| 156 | ttGGBP17C.29 Exemplary Affinity-Tuning mutant (17C Background) |
| | Expression Construct (Table 6) |
| 157 | ttGGBP182C.2.0 Exemplary Affinity-Tuning mutant (182C Background) |
| | Expression Construct (Table 6) |
| 158 | ttGGBP182C.2.1 Exemplary Affinity-Tuning mutant (182C Background) |
| | Expression Construct (Table 6) |
| 159 | ttGGBP182C.2.3 Exemplary Affinity-Tuning mutant (182C Background) |
| | Expression Construct (Table 6) |
| 160 | ttGGBP182C.2.4 Exemplary Affinity-Tuning mutant (182C Background) |
| | Expression Construct (Table 6) |
| 161 | ttGGBP182C.2.5 Exemplary Affinity-Tuning mutant (182C Background) |
| | Expression Construct (Table 6) |
| 162 | ttGGBP182C.2.6 Exemplary Affinity-Tuning mutant (182C Background) |
| | Expression Construct (Table 6) |
| 163 | ttGGBP182C.2.7 Exemplary Affinity-Tuning mutant (182C Background) |
| | Expression Construct (Table 6) |
| 164 | ttGGBP182C.2.8 Exemplary Affinity-Tuning mutant (182C Background) |
| | Expression Construct (Table 6) |
| 165 | ttGGBP182C.2.9 Exemplary Affinity-Tuning mutant (182C Background) |
| | Expression Construct (Table 6) |
| 166 | ttGGBP182C.3 Exemplary Affinity-Tuning mutant (182C Background) |
| | Expression Construct (Table 6) |
| 167 | ttGGBP182C.4 Exemplary Affinity-Tuning mutant (182C Background) |
| | Expression Construct (Table 6) |
| 168 | ttGGBP182C.5 Exemplary Affinity-Tuning mutant (182C Background) |
| | Expression Construct (Table 6) |
| 169 | ttGGBP182C.6 Exemplary Affinity-Tuning mutant (182C Background) |
| | Expression Construct (Table 6) |
| 170 | ttGGBP182C.7 Exemplary Affinity-Tuning mutant (182C Background) |
| | Expression Construct (Table 6) |
| 171 | ttGGBP182C.8 Exemplary Affinity-Tuning mutant (182C Background) |
| | Expression Construct (Table 6) |
| 172 | ttGGBP182C.9 Exemplary Affinity-Tuning mutant (182C Background) |
| | Expression Construct (Table 6) |
| 173 | ttGGBP182C.2.0_Imm0 |
| 174 | ttGGBP, R91K, Q148E |
| 175 | ttGGBP182C.2.0_Imm1 |
| 176 | ttGGBP182C.2.0_Imm2 |
| 177 | ttGGBP182C.2.0_Imm3 |
| 178 | ttGGBP182C.2.0_Imm4 |
| 179 | ttGGBP182C.2.0_Imm5 |
| 180 | ttGGBP182C.2.0_Imm6 |
| 181 | ttGGBP182C.2.0_Imm7 |
| 182 | IYKXDDXFM (Conserved Sequence) |
| 183 | YKXDDXFM (Conserved Sequence) |
| 184 | YKXDDXF (Conserved Sequence) |
| 185 | KXDDXF (Conserved Sequence) |
| 186 | YKXDD (Conserved Sequence) |
| 187 | KXDD (Conserved Sequence) |
| 188 | DDXF (Conserved Sequence) |
| 189 | DXFMXXVR (Conserved Sequence) |
| 190 | DXFMXXV (Conserved Sequence) |
| 191 | DXFM (Conserved Sequence) |
| 192 | IYKXDNXFM (Conserved Sequence) |
| 193 | YKXDNXFM (Conserved Sequence) |
| 194 | YKXDNXF (Conserved Sequence) |
| 195 | KXDNXF (Conserved Sequence) |
| 196 | YKXDN (Conserved Sequence) |
| 197 | KXDN (Conserved Sequence) |
| 198 | DNXF (Conserved Sequence) |
| 199 | NXFMXXVR (Conserved Sequence) |
| 200 | NXFMXXV (Conserved Sequence) |
| 201 | NXFM (Conserved Sequence) |
| 202 | PVVFFNKEP (Conserved Sequence) |
| 203 | PVVFXNKEP (Conserved Sequence) |
| 204 | PVVFFNXEP (Conserved Sequence) |
| 205 | PVVFXNXEP (Conserved Sequence) |
| 206 | VVFXNXEP (Conserved Sequence) |
| 207 | VFXNXEP (Conserved Sequence) |
| 208 | PVVFXNXE (Conserved Sequence) |
| 209 | PVVFXN (Conserved Sequence) |
| 210 | PXVFXN (Conserved Sequence) |
| 211 | PVXFXN (Conserved Sequence) |
| 212 | FXNXEP (Conserved Sequence) |
| 213 | FXNXE (Conserved Sequence) |
| 214 | PGHPDAEART (Conserved Sequence) |
| 215 | PGHXDAXXRT (Conserved Sequence) |
| 216 | GHXDAXXRT (Conserved Sequence) |
| 217 | HXDAXXRT (Conserved Sequence) |
| 218 | DAXXRT (Conserved Sequence) |
| 219 | PGHXDAXXR (Conserved Sequence) |
| 220 | PGHXDA (Conserved Sequence) |
| 221 | PGHXD (Conserved Sequence) |
| 222 | PGNPDAEART (Conserved Sequence) |
| 223 | PGNXDAXXRT (Conserved Sequence) |
| 224 | GNXDAXXRT (Conserved Sequence) |
| 225 | NXDAXXRT (Conserved Sequence) |
| 226 | PGNXDAXXR (Conserved Sequence) |
| 227 | PGNXDA (Conserved Sequence) |
| 228 | PGNXD (Conserved Sequence) |
| 229 | DTAMWD (Conserved Sequence) |
| 230 | DTAMCD (Conserved Sequence) |
| 231 | DTAMW (Conserved Sequence) |
| 232 | DTAMC (Conserved Sequence) |
| 233 | TAMWD (Conserved Sequence) |
| 234 | TAMCD (Conserved Sequence) |
| 235 | AXWXX (Conserved Sequence) |
| 236 | AXCXX (Conserved Sequence) |
| 237 | IEVVIANND (Conserved Sequence) |
| 238 | EVVIANND (Conserved Sequence) |
| 239 | IEVVIANN (Conserved Sequence) |
| 240 | EVVIANN (Conserved Sequence) |
| 241 | IEXVXXNND (Conserved Sequence) |
| 242 | IEXVXXNN (Conserved Sequence) |
| 243 | EXVXXNND (Conserved Sequence) |
| 244 | EXVXXNN (Conserved Sequence) |
| 245 | PVFGVDA (Conserved Sequence) |
| 246 | VFGVDA (Conserved Sequence) |
| 247 | PVFGVD (Conserved Sequence) |
| 248 | FGVDA (Conserved Sequence) |
| 249 | PVXGVDA (Conserved Sequence) |
| 250 | VXGVDA (Conserved Sequence) |
| 251 | PVXGVD (Conserved Sequence) |
| 252 | VXGVD (Conserved Sequence) |
| 253 | GTVLNDA (Conserved Sequence) |
| 254 | GTVLND (Conserved Sequence) |
| 255 | GTVLN (Conserved Sequence) |
| 256 | TVLND (Conserved Sequence) |
| 257 | chyGGBP_F12C (chGGBP with signal sequence replaced with M; F12C, |
| | C39A, and C206A mutations; and GGSHHHHHH at C-terminus) |
| 258 | chyGGBP_F16C (chGGBP with signal sequence replaced with M; F16C, |
| | C39A, and C206A mutations; and GGSHHHHHH at C-terminus) |
| 259 | chyGGBP_W181C (chGGBP with signal sequence replaced with M; |
| | W181C, C39A, and C206A mutations; and GGSHHHHHH at C-terminus) |
| 260 | cobGGBP_F12C (cobGGBP with signal sequence replaced with M; |
| | F12C, C39A, C173A, and C206A mutations; and GGSHHHHHH at C- |
| | terminus) |
| 261 | cobGGBP_F16C (cobGGBP with signal sequence replaced with M; |
| | F16C, C39A, C173A, and C206A mutations; and GGSHHHHHH at C- |
| | terminus) |
| 262 | cobGGBP_W181C (cobGGBP with signal sequence replaced with M, |
| | W181C, C39A, C173A, and C206A mutations; and GGSHHHHHH at C- |
| | terminus) |
| 263 | pspGGPB_F13C (pspGGBP with signal sequence replaced with M; F13C |
| | mutation; and GGSHHHHHH at C-terminus) |
| 264 | pspGGPB_F9C (pspGGBP with signal sequence replaced with M; F9C |
| | mutation; and GGSHHHHHH at C-terminus) |
| 265 | pspGGPB_W178C (pspGGBP with signal sequence replaced with M; |
| | W178C mutation; and GGSHHHHHH at C-terminus) |
| 266 | csaGGBP_F18C (csaGGBP with signal sequence replaced with M; F18C, |
| | C62A, C82A, C113A, and C211A mutations; and GGSHHHHHH at C- |
| | terminus) |
| 267 | csaGGBP_W186C (csaGGBP with signal sequence replaced with M; |
| | W186C, C62A, C82A, C113A, and C211A mutations; and GGSHHHHHH |
| | at C-terminus) |
| 268 | csaGGBP_Y14C (csaGGBP with signal sequence replaced with M; |
| | Y14C, C62A, C82A, C113A, and C211A mutations; and GGSHHHHHH at |
| | C-terminus) |
| 269 | bprGGBP_F16C (bprGGBP with signal sequence replaced with M; |
| | F16C, C8A, C112A, C116A, C179A, C211A, and C289A mutations; and |
| | GGSHHHHHH at C-terminus) |
| 270 | bprGGBP_K12C (bprGGBP with signal sequence replaced with M; |
| | K12C, C8A, C112A, C116A, C179A, C211A, and C289A mutations; and |
| | GGSHHHHHH at C-terminus) |
| 271 | bprGGBP_W187C (bprGGBP with signal sequence replaced with M; |
| | W187C, C8A, C112A, C116A, C179A, C211A, and C289A mutations; |
| | and GGSHHHHHH at C-terminus) |
| 272 | rinGGBP_A_F10C (rinGGBP_A with signal sequence replaced with M; |
| | F10C, C6A, C114A, C177A, C210A, and C288A mutations; and |
| | GGSHHHHHH at C-terminus) |
| 273 | rinGGBP_A_F14C (rinGGBP_A with signal sequence replaced with M; |
| | F14C, C6A, C114A, C177A, C210A, and C288A mutations; and |
| | GGSHHHHHH at C-terminus) |
| 274 | rinGGBP_A_W185C (rinGGBP_A with signal sequence replaced with |
| | M; W185C, C6A, C114A, C177A, C210A, and C288A mutations; and |
| | GGSHHHHHH at C-terminus) |
| 275 | rinGGBP_B_F17C (rinGGBP_B with signal sequence replaced with M; |
| | F17C, C66A, C70A, and C306A; and GGSHHHHHH at C-terminus) |
| 276 | rinGGBP_B_Q13C (rinGGBP_B with signal sequence replaced with M; |
| | Q13C, C66A, C70A, and C306A mutations; and GGSHHHHHH at C- |
| | terminus) |
| 277 | rinGGBP_B_W189C (rinGGBP_B with signal sequence replaced with M; |
| | W189C, C66A, C70A, and C306A mutations; and GGSHHHHHH at C- |
| | terminus) |
| 278 | fprGGBP_F12C (fprGGBP with signal sequence replaced with M; F12C, |
| | C8A, C105A, C106A, C143A, and C205A mutations; and GGSHHHHHH |
| | at C-terminus) |
| 279 | fprGGBP_F16C (fprGGBP with signal sequence replaced with M; F16C, |
| | C8A, C105A, C106A, C143A, and C205A mutations; and GGSHHHHHH |
| | at C-terminus) |
| 280 | fprGGBP_W180C (fprGGBP with signal sequence replaced with M; |
| | W180C, C8A, C105A, C106A, C143A, and C205A mutations; and |
| | GGSHHHHHH at C-terminus) |
| 281 | cljGGBP_F11C (cljGGBP with signal sequence replaced with M; F11C, |
| | C77A, and C198A mutations; and GGSHHHHHH at C-terminus) |
| 282 | cljGGBP_W15C (cljGGBP with signal sequence replaced with M; |
| | W15C, C77A, and C198A mutations; and GGSHHHHHH at C-terminus) |
| 283 | cljGGBP_W176C (cljGGBP with signal sequence replaced with M; |
| | W176C, C77A, and C198A mutations; and GGSHHHHHH at C-terminus) |
| 284 | cauGGBP_F12C (cauGGBP with signal sequence replaced with M; |
| | F12C, C78A, and C199A mutations; and GGSHHHHHH at C-terminus) |
| 285 | cauGGBP_W16C (cauGGBP with signal sequence replaced with M; |
| | W16C, C78A, and C199A mutations; and GGSHHHHHH at C-terminus) |
| 286 | cauGGBP_W177C (cauGGBP with signal sequence replaced with M; |
| | W177C, C78A, and C199A mutations; and GGSHHHHHH at C-terminus) |
| 287 | erhGGBP_F13C (erhGGBP with signal sequence replaced with M; F13C |
| | mutation; and GGSHHHHHH at C-terminus) |
| 288 | erhGGBP_F17C (erhGGBP with signal sequence replaced with M; F17C |
| | mutation; and GGSHHHHHH at C-terminus) |
| 289 | erhGGBP_W188C (erhGGBP with signal sequence replaced with M; |
| | W188C mutation; and GGSHHHHHH at C-terminus) |
| 290 | ereGGBP_F17C (ereGGBP with signal sequence replaced with M; F17C, |
| | C10A, C29A, C65A, C69A, and C183A mutations; and GGSHHHHHH at |
| | C-terminus) |
| 291 | ereGGBP_Q13C (ereGGBP with signal sequence replaced with M; |
| | Q13C, C10A, C29A, C65A, C69A, and C183A mutations; and |
| | GGSHHHHHH at C-terminus) |
| 292 | ereGGBP_W188C (ereGGBP with signal sequence replaced with M; |
| | W188C, C10A, C29A, C65A, C69A, and C183A mutations; and |
| | GGSHHHHHH at C-terminus) |
| 293 | βZif |
| 294 | ZF-QNK |
| 295 | Hexahistidine Tag |
| 296 | Hexalysine Tag |
| 297 | ecGGBP.F16C (with signal peptide replaced with M; a F16C mutation; |
| | and a GGSHHHHHH at C-terminus) |
| 298 | ttGGBP182C.2.0 (affinity-tuning mutant, 182C background (Table 6) |
| | with signal peptide replaced with M) |

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1. Fluorescently Responsive Sensor Engineering Phases

The engineering of FRSs can be divided into five phases:
1. Binding protein discovery. A set of glucose-binding protein sequence homologs is identified. Accurate assignment of their ligand-binding function utilizes application of a prediction method that incorporates information encoded in the experimentally determined three-dimensional structure of periplasmic glucose-binding proteins.
2. Experimental lead validation. Synthetic genes are constructed, which are optimized for heterologous expression in Escherichia coli of one or more predicted glucose-binding protein sequences. The glucose-binding properties and thermostabilities of the corresponding expressed, purified proteins are evaluated.
3. Engineering of fluorescent responses. Semisynthetic fluorescent conjugates of the experimentally validated leads are constructed by attaching fluorophores to single cysteine mutants. The effect of glucose binding on the fluorescence emission properties of those conjugates is evaluated. Those conjugates that evince strong, ratiometric responses are selected as FRSs.
4. Affinity tuning. Single or multiple mutations are introduced by site-directed mutagenesis to alter the glucose-binding affinities of glucose-responsive FRSs. A set of FRS variants is selected that together cover the clinical glucose concentration range with high accuracy.
5. Device integration. FRSs are immobilized in the sampling component of the analytical device in a manner that preserves their fluorescent response and glucose affinity. Long-term storage conditions are established.

### Example 2. Identification of a Family of Periplasmic Glucose-Binding Proteins Homologs Using Structurally Assisted Function Evaluation (Phase Step 1)

As a first step in constructing robust glucose sensor candidates, we examined bacterial genomic sequences to identify periplasmic glucose-binding protein sequences in known thermophiles. Homologs from such organisms are likely to encode thermostable proteins. Analysis of enzyme families has shown that overall sequence identity below ˜60% is a weak predictor of function conservation (Todd 2001 J. Mol. Biol., 307, 1113-1143; Tian 2003 J. Mol. Biol., 333, 863-882). Furthermore, functional assignments based on sequence homology alone are known to be particularly problematic in the PBP superfamily. For instance, PBPs that by overall sequence identity are predicted to bind oligopeptides were found to bind oligosaccharides. Enzyme functional assignments are improved greatly if a sequence selection filter based on conservation of catalytic residues identified from protein structures is included. Such catalytic residues comprise a subset of all the residues that contact an enzyme substrate or inhibitor. In the case of the PBPs, functional selection filters need to take into account all the protein-ligand contacts that encode the ligand-binding function. Accordingly, we have developed a structurally assisted functional evaluation (SAFE) method to identify PBP sequence homologs with accurately predicted function. The SAFE homolog search method consists of five steps encoded in the ProteinHunter software package:
1. Sequence homolog set is collected using the BLAST sequence alignment tool (Altschul et al. 1990) with standard settings (expect threshold: 10.0; gap cost existence: 11; gap extension: 1; substitution matrix: Blosum62; filter for low complexity regions on), starting with Escherichia coli periplasmic glucose-galactose binding protein (ecGGBP) sequence (SEQ ID NO: 17) as a seed (Vyas, Vyas and Quiocho 1988 Science, 242, 1290-5). The ProteinHunter program selects only those pairwise BLAST alignments that (a) share a minimum of 20% sequence identity, and (b) have 70% coverage of their entire sequence length by the aligned region. This set of sequences defines a universe of possible glucose-binding proteins without assigning function.
2. Structure-based encoding of biological function. A PCS comprising the protein residues that form hydrogen bonds and van der Waals contacts with the bound glucose is defined using computer-assisted, visual inspection of the three-dimensional structure of the ecGGBP-glucose complex. ProteinHunter identifies if the calculated distance between any of their atoms and any glucose atom is less than 5 Å, and the distances between their backbone C_{α} and any atom in glucose is greater than that of their C_{β} atom and any atom in glucose. Secondary shell residues that do not form hydrogen bonds or van der Waals contacts are removed by inspection from the resulting set. The choice of allowed residues is guided by the identity of the residue in the structure, and mutants that are likely to maintain that interaction. The resulting definition specifies residue positions and their permitted amino acid identity. Multiple amino acid identities are permitted at each position to encode functionally equivalent residues. This definition establishes a search filter for the accurate prediction of glucose-binding proteins within the universe of sequence homologs collected in (1).
3. Accurate sequence alignment. Tools such as ClustalW (Chenna et al. 2003 Nucleic Acids Res, 31, 3497-500) are used to construct an accurate alignment of all the sequence homologs. The ecGGBP seed sequence is included in this alignment. This multiple sequence alignment establishes the equivalent positions of the ecGGBP PCS in each sequence homolog.
4. Function evaluation. The glucose-binding properties of each of the aligned sequence homologs is determined by measuring their compliance with the PCS sequence filter. A “Hamming distance”, H, is assigned for each homolog by the ProteinHunter program, which specifies the degree of sequence identity of all the residues at the aligned PCS positions. A value of H=0 indicates that the identities of all the residues at the aligned PCS positions match the amino acid(s) allowed in the PCS search filter; H>0, indicates that one or more aligned positions have disallowed residues. Sequences for which H=0 are predicted to encode glucose-binding proteins.
5. Selection of representative SAFE homologs. The sequence homologs are ordered by (a) identity with the seed PCS, as measured by the Hamming distance, (b) fractional overall sequence identity with the seed sequence. A subset for sequences with H=0, sampling the fractional overall sequence identity is selected for experimental verification.

The ProteinHunter software tool encodes the flow of execution, applies the PCS search filter, and visualizes the results that include organism annotations such as thermophilicity, and Gram stain status.

These steps are encoded in the ProteinHunter software tool, which encodes the flow of execution, applies the PCS search filter, and visualizes the results, and handles organism annotations such as thermophilicity, and Gram stain status.

Annotated genomic and plasmid sequences of 5062 prokaryotes were downloaded from the National Center of Biotechnology Information (download date: May 17, 2015; ftp://ftp.ncbi.nih.gov/genomes/Bacteria/all.gbk.tar.gz). The protein sequence for the E. coli glucose-galactose binding protein (ecGGBP) was extracted from the protein structure file 2gbp (Vyas et al. 1988 Science, 242, 1290-5), and used as the seed sequence for the BLAST search described above. A total of 1822 sequence homologs were identified.

In ecGGBP, glucose binding is encoded by a PCS comprising ten residues. Two aromatic residues (F16, W182) sandwich the bound glucose through extensive van der Waals interactions with either surface of the pyranose ring. The other eight residues form hydrogen bonds with all the glucose hydroxyls. A PCS filter specifying multiple amino acids at these 10 positions was used to predict glucose-binding proteins (FIGS. 2A-C). A total of 139 homologs were predicted to encode glucose-binding proteins, on the basis of their Hamming distance scores (H=0). The overall sequence identities of these homologs relative to the ecGGBP seed varied from 100% to 29% (Table 1).

### Example 3. Lead Protein Validation Using Ligand-Mediated Thermostability Shifts (Phase Step 2)

Thirteen homologs that were predicted to be glucose-binding proteins (FIG. 3) were selected to probe different degrees of sequence identity to the ecGGBP seed, and their glucose-binding properties were determined experimentally (Table 2). These experiments comprise four successive steps:
1. Synthetic gene construction. The amino acid sequence of the homology leads are backtranslated into DNA sequences. These are optimized for directing heterologous cytoplasmic expression of the protein homologues in E. coli, using either the OrfOpt, (U.S. Patent Publication No. 2011/0171737), hereby incorporated by reference or OrfMorph (described herein) programs. These programs predict mRNA sequences that direct high-level protein expression in E. coli. The predicted gene sequences are assembled de novo from synthetic oligonucleotides.
2. Heterologous protein expression of the homologues in E. coli. Plasmids carrying the synthetic expression constructs (see above) were transformed into KRX (E-coli K12 Derivative Strain) competent cells (Promega, Technical Bulletin TB352). Protein production was induced in bacterial cultures of these cultures, as described in the Materials and Methods.
3. Purification of successfully expressed protein using immobilized metal affinity chromatography, as described in Materials and Methods.
4. Verification of glucose binding. Determination of the glucose-binding properties of the purified proteins using a thermal stability shift assay, as described in Materials and Methods.
   Seven of the thirteen synthetic expression constructs successfully directed heterologous cytoplasmic expression of a GGBP homolog in E. coli. The glucose-binding properties of five of these were confirmed directly using the thermal shift assay (FIGS. 4A-B). The thermostability of two homologs was too high (^{apo}Tₘ>90° C.) for this assay to be applicable. Their glucose-binding properties were confirmed by measuring the temperature-dependent fluorescence responses of Acrylodan-labeled single cysteine mutants (see section 5) to glucose (FIGS. 5A-B).

Surprisingly, the sequence identity of all these experimentally verified glucose-binding homologs relative the ecGGBP seed are considerably below the 60% threshold, ranging from 29% to 48%. Several use alternate amino acids in the PCS at position 152, demonstrating the predictive power of allowing functionally equivalent residues. These results therefore demonstrate that biological function can be predicted accurately with the SAFE technique, even in sequence homologs with low fractional identities to the original seed.

Three of the experimentally verified GGBP homologs exhibit mid-point thermal denaturation temperatures (Tₘ values) in the 347-350 K range, which is at least 25 K more stable than the ecGGBP seed (Tₘ=322 K). Of these, the homolog from Thermoanaerobacter thermosaccharolyticum (ttGGBP) was produced at the highest level by heterologous expression in E. coli. This protein was selected as the candidate for constructing robust glucose sensors.

### Example 4. Cysteine Mutant Scans and Fluorophore Screening to Identify Fluorescently Responsive Glucose Sensors (Phase Step 3)

Semi-synthetic FRSs were engineered by site-specifically attaching thiol-reactive, environmentally sensitive fluorophores that respond to ligand-mediated conformational changes. Identification of FRS candidates that are useful for sensing applications comprises three steps:
1. Cysteine scan. Mutant glucose-binding proteins containing single cysteines are constructed for site-specific attachment of thiol-reactive fluorophores. Positions in PBPs where attached single fluorophores are likely to exhibit ligand-dependent responses were determined (de Lorimier et al. 2002 Protein Sci, 11, 2655-75). Candidate positions fall into three classes: endosteric, replacing a residue that contacts the ligand directly; peristeric, located at the rim of the binding site; allosteric (Marvin et al. 1997 Proc Natl Acad Sci USA, 94, 4366-71; Marvin 1998 J Am Chem Soc, 120, 7-11), located outside the binding site at sites that undergo local structural changes in concert with the hinge-bending motion.
2. Fluorophore screening. Thiol-reactive, environmentally sensitive fluorophores are attached to each cysteine mutant prepared in step 1.
3. Evaluation of the glucose-mediated change of all the fluorescent conjugates prepared in step 2. Responses to ligand binding in which there is both a change in fluorescence emission intensity and spectral shape was essential for chemometric applications, because such changes enable ratiometric measurements. Changes in spectral shape typically are accompanied by a shift in the wavelength of the emission intensity maxima. Three classes of fluorescent responses are possible:
   i. No response.
   ii. Monochromatic response (emission intensity increases or decreases without a change in spectral shape)
   iii. Dichromatic response (both intensity and spectral shape changes) which can be classified into two sub-classes:
      i. Hypsochromatic: emission intensity shifts to shorter wavelengths upon binding glucose (“blue shift”).
      ii. Bathochromatic: emission intensity shifts to longer wavelengths upon binding glucose (“red shift”).

We constructed seventeen single cysteine mutants in ttGGBP, exploring four endosteric, ten peristeric, and three allosteric positions (FIFA. 6A-B). At each position, we attached the Prodan-derived fluorophores such as Acrylodan and Badan, which differ by one methylene group in their thiol-reactive linking moiety. The fluorescence emission intensities of seven Acrylodan and five Badan conjugates responded to glucose at seven attachment positions (Table 3). At only four attachment positions were the responses of both fluorophores qualitatively similar, and never quantitatively. We also tested for glucose binding by measuring ligand-mediated shifts in protein thermal stability (Table 3). By this criterion, the majority of the non-responsive conjugates bound glucose. The two conjugates that did not bind glucose (N42C⋅Badan, V67C⋅Badan) also exhibited large losses in overall thermostability (28-30 K decrease in apo Tₘ values), indicating that the attached fluorophore adversely affected their structural integrity. By contrast, all other conjugates exhibited only small variations in apo-protein thermostability (1-4 K), indicating that the fluorophore causes minimal perturbations instability.

Endosteric attachment positions exhibited the most pronounced changes in fluorescence emissions in response to ligand binding. Conjugates at three of the ten peristeric positions were responsive to glucose. No allosteric conjugates exhibited fluorescence responses to glucose.

We observed ligand-dependent shifts in the wavelengths of emission intensity maxima at one peristeric (Y11C) and two endosteric (F17C, W182C) sites (FIGS. 7A-F), enabling dichromatic ratiometric measurements; the maximum intensity of other glucose-responsive conjugates remained the same (monochromatic responses). Wavelengths shifts occurred in both directions: in the ttGGBP W182C⋅Acrylodan conjugate the apo-protein spectrum converted from a short- to a long-wavelength emission state upon binding glucose (bathochromic shift), whereas the emissions in F17C⋅Acrylodan, F17C⋅Badan and Y11C⋅Badan conjugates shifted to shorter wavelengths (hypsochromic shift). The Badan and Acrylodan conjugates attached to F17C and W182C, respectively, exhibited the largest, wavelength-dependent changes in fluorescence emission intensities, (FIGS. 7A-C).

The responses to glucose of a number of other fluorophores (FIGS. 8A-P) were also tested at positions 182C and 17C (Table 4). Several conjugates exhibited significant changes in emission intensity upon glucose addition, but none of these were accompanied by large wavelengths shifts of their maxima. Both increases and decreases in fluorescence were observed.

The most effective sensors that were discovered are the Badan and Acrylodan conjugates attached to the endosteric cysteine mutations F17C and W182C, respectively. For example, peptides with SEQ ID No:61 (ttGGBP182C), SEQ ID NO:88 (ttGGBP182C.2.0), SEQ ID NO:90 (ttGGBP182C.2.3), and SEQ ID NO:102 (ttGGBP182C.8) are particularly useful for clinical diagnostics, because they span hypoglycemia, euglycemia, mild hyperglycemia, severe hyperglycemia, and hyperosmolar hyperglycemic glucose concentrations respectively. These two fluorophores differ only in their linker geometry, but this small difference determines whether dichromatic or monochromatic responses are observed for a particular conjugate. At position 17C, only the Badan and not the Acrylodan conjugate evinces a dichromatic response; at 182C; surprisingly, the reverse case is observed. Within a given bacterial periplasmic binding protein, some attachment sites are more effective than others, and within a given attachment site, some fluorophores are more effective than others (DeLorimier et al., 2002, Protein Science 11:2655-2675). Thus, the nature of the responses therefore is idiosyncratic, and therefore the choice of which site to mutate (e.g., substitute with a single cysteine) and which fluorophore to use at a particular site is nonobvious. Changes in linker geometry and chromophore modifications give rise to significant differences in the detailed interactions of particular fluorophores with the protein, even within families of closely related molecules, thereby significantly impacting sensor characteristics, consistent with previous observations.

### Example 5: Determination of Exemplary Fluorescent Conjugate Structures

The structures of the fluorescent conjugates with pronounced dichromatic responses, ttGGBP17C⋅Badan and ttGGBP182C.2.0⋅Acrylodan (SEQ ID NO: 88) (this is a variant in which the glucose-binding affinity has been manipulated), were determined by X-ray crystallography (Table 5; FIG. 9). The structure of the E. coli GGBP homolog conjugate, ecGGBP W183C⋅Acrylodan (ecGGBP W183C, SEQ ID NO: 32), was determined also.

Like all members of the periplasmic-binding protein (PBP) superfamily, GGBPs comprise two domains connected by a flexible hinge, with the ligand-binding site located in a cleft between the two domains. In the absence of ligand, PBPs adopt an open conformation, in which the two domains move apart and the cleft is wide. In ligand complexes, hinge-bending motions move the two domains closer together, forming a binding site that completely envelops the ligand, reminiscent of a protein interior. The overall structures of the ecGGBP and ttGGBP conjugates are similar with a backbone C_{α} RMSD of 0.6 Å and 0.9 Å for the N- and C-terminal domains respectively (C-terminal domain contains the Ca²⁺ binding site) as expected for proteins that are 48% identical (Chothia 1986 EMBO J., 5, 823-826). Small variations in the degree of closure can affect critical inter-domain hydrogen-bonding interactions. With the exception of mutated aromatic groups, the interactions between the protein and bound glucose are conserved. The structures of these conjugates enabled us to determine the interactions between the protein and the fluorophore and the internal conformation of the fluorophore. These sets of observations guided the choice of mutations for manipulating glucose affinities.

In both F17C⋅Badan and W182C⋅Acrylodan, the conjugated fluorophores point out into solution. A view down the long axis of ttGGBP from the C-terminal towards the N-terminal domain into the ligand-binding site reveals that three channels, each approximately 120° apart, connect the centrally located bound monosaccharide to the protein surface (FIG. 9c). One channel is occupied by the R-group of the natural galactoside ligand (this position is occupied by water in the glucose and galactose complexes), the other two are filled by Badan and Acrylodan in their respective conjugates.

In the wild-type protein, the rings of F17 and W182 form extensive van der Waals contact with the bound glucose. The outward orientation of the fluorophore in the W182C⋅Acrylodan conjugate leaves a cavity vacated by the indole ring, which is filled with water and cryoprotectant. The cavity created by the loss of the smaller benzyl ring in F17C⋅Badan is largely filled with the linker; no water or cryoprotectant was observed.

The Acrylodan and Badan fluorophores contain two important internal degrees of freedom (FIGS. 6a-c): the twist of the dimethylamino group, and the carbonyl. The state of these two angles influences the fluorescence properties of the fluorophore, by altering the extent of electronic conjugation within the system, and the degree of polarization of the excited dipole. The electron density of Badan in the ttGGBP F17C conjugate was well defined, enabling us to determine the internal fluorophore conformation with high confidence (FIG. 9d-f). The dimethyl amino group is co-planar with the naphthalene ring. The carbonyl group is twisted out of plane by approximately 30°. By contrast, in ecGGBP.183C⋅Acrylodan the carbonyl is co-planar (FIG. 9g). This group is probably also co-planar in the ttGGBP.182C. Acrylodan structure, but the partial occupancy of the conjugate in this structure precluded a definitive conclusion. Nevertheless, the high degree of similarity in structure and spectral properties of the ecGGBP 183C and ttGGBP 182C Acrylodan conjugates suggests that the Acrylodan carbonyl is co-planar in both homolog conjugates.

In both ttGGBP 17C⋅Badan and 182C⋅Acrylodan (and ecGGBP 183C⋅Acrylodan) conjugates, the pocket in which the fluorophore carbonyl is located comprises residues contributed by each domain, and is fully formed only in the closed, glucose-bound protein conformation. No hydrogen bonds are observed between the fluorophore carbonyl and the protein at either attachment position. In ttGGBP 17C⋅Badan the “knob” of the twisted carbonyl conformation is bound in a small “hole” formed by residues also contributed by both domains (FIG. 9h). The carbonyl is held in place by van der Waals interactions proximal to the attachment site with residues located in the domain opposite to the F17C site: the CB carbon of N258 touches the carbonyl; the backbone atoms of residues 239-241 interact with one of the two naphthalene rings faces. The twisted ttGGBP F17C⋅Badan conformation therefore is stabilized by interactions of the protein with both the naphthalene ring and the carbonyl. These interactions are fully formed in the closed state only. By contrast, in ecGGBP 183C⋅Acrylodan the carbonyl hole is not well defined. The carbonyl is flanked by E149 (same domain as attachment site), and K92 (opposite domain), but these residues form no direct contacts in the structure. Neither the carbonyl nor the naphthalene rings of this planar conjugate are constrained by clear interactions in the protein closed conformation.

### Example 6. Affinity Tuning (Phase Step 4)

Blood glucose concentrations range from ˜3 mM (hypoglycemia) to ˜30 mM (hyperglycemia) and up to ˜100 mM for the hyperosmolar hyperglycemic state (HHS), with healthy levels at around 6 mM (euglycemia). Measurements using reagentless sensors are most sensitive at analyte concentrations that match the dissociation constant. The glucose affinity of ttGGBP182C⋅Acrylodan is too high and must therefore be “tuned” by raising the K_{d} value.

The mutations that alter glucose affinities fall into four classes:
1. Alteration of direct interactions in the PCS between the protein and the bound glucose.
2. Manipulation of the equilibrium between the open and closed states.
3. Alteration of interactions between the protein and the fluorescent conjugate. Two sub-classes can be distinguished:
   a. Interactions with the carbonyl group
   b. Interactions with the naphthalene ring
4. Indirect interactions that alter the geometry of the binding site.
   Representatives of mutant classes 1-3 were constructed in the ttGGBP182C and ttGGBP17C backgrounds, using Acrylodan and Badan conjugates to evaluate their effects on glucose binding (Table 6).

Mutations in the PCS residues (Class 1) do not afford many opportunities for manipulating affinity subtly. Mutagenesis of ecGGBP has demonstrated that most PCS positions are intolerant of mutations, with the exception of histidine that interacts with the 6-hydroxyl, and the aspartate that binds the epimeric 4-hydroxyl. In ttGGBP182C⋅Acrylodan, the H151Q mutations subtly lower the affinity into the middle of the pathophysiological concentration range, whereas in ttGGBP17C⋅Badan H151Q lowers affinity, but nearly abolishes ratiometry. In ttGGBP182C⋅Acrylodan, D15N weakens glucose binding into the HHS range and increases discrimination between glucose and galactose. In ttGGBP17C⋅Badan, D15A weakens ligand binding and enhances the dichromatic response.

Uniquely in ttGGBP182C⋅Acrylodan, the PCS also can be manipulated by mutating position 154 adjacent to the cavity vacated by the missing indole ring of W182 (FIG. 10a). Three mutations in Ala154 alter the affinity 50-fold, spanning the entire pathophysiological glucose concentration range. The A154M mutation increases the affinity ˜10-fold, presumably by (partially) restoring direct van der Waals interactions between the protein and bound glucose, which were lost upon removal of the tryptophan. The 154S mutation causes a 19 K decrease in thermostability; the other mutations exhibit negligible effects on thermostability.

The conformational equilibrium can be altered by manipulating those hydrogen bonds between the N- and C-terminal domains that are predicted to form in the closed, but not the open state (Class 2 mutations). Four locations form inter-domain hydrogen bonds in the protein closed conformation (FIG. 10b) of ttGGBP: T16-D211, R69-G150 (main-chain), R91-Q148, and E92 (main-chain)-Q152. The first of these interactions is located between the Badan and Acrylodan channels, whereas the other three are positioned between the Acrylodan and galactoside R-group channels (FIG. 9c). Disruption of inter-domain interactions alters the intrinsic equilibrium between the open and closed conformations, thereby decreasing ligand affinity. Accordingly, in the ttGGBP W182C⋅Acrylodan conjugate mutants, glucose affinities are lowered (K_{d} values raised) in the class 2 mutations. These mutations also alter the fluorescence emission spectra. In all the class 2 mutants of both ttGGBPF17C Acrylodan and Badan conjugates the spectral responses are altered, either enhancing or abolish wavelength shifts. These observations indicate that mutations of the inter-domain hydrogen bonds cause (small) changes in the structure of the closed state, which affect the fluorophore environment.

Mutations in the interactions between the protein and the conjugated fluorophore (Class 3 mutants), changing either the carbonyl hole (Class 3a) or the fluorophore channel walls (Class 3b), have large effects on glucose binding and fluorescence spectra. In ttGGBP17C⋅Badan, most of the N258 mutants in the carbonyl hole do not respond to glucose. The absence of shifts in thermal stability in the presence of glucose indicates that these mutants no longer bind ligand. The 17C⋅Acrylodan N258S mutant conjugate is the exception: it binds and responds to glucose, but binding does not evoke a fluorescent response. Mutations in residues located on either side of the channel wall strongly influence spectral properties of the fluorescent conjugates. In ttGGBP17C⋅Badan, with the exception of A260W, mutations in A260 convert dichromatic to monochromatic responses. In ttGGBP182C⋅Acrylodan, the behavior of mutations in the R91 and Q148 interaction is complex, because these potentially affect both inter-domain hydrogen bonding and interactions between the protein and the conjugated fluorophore. The fluorophore blocks the formation of R91K-Q148E in the ttGGBP182C.2.0⋅Acrylodan double mutant structure, but in ecGGBP183C⋅Acrylodan E149-K92 is present, reflecting subtle differences in fluorophore conformation and inter-domain closure angles. The two single Q148E and R91K mutants both exhibit a modest increase of glucose affinities in ttGGBP182C⋅Acrylodan, suggesting that this inter-domain is maintained, and slightly improved. By contrast, the Q148E+R91K double mutant exhibits a 2.6-fold decrease in affinity, consistent with the observed loss of interaction in the ttGGBP182C2.0⋅Acrylodan double mutant structure (SEQ ID NO: 88). The glucose affinity of the ttGGBP182C.2.0⋅Acrylodan double mutant (SEQ ID NO: 88) falls within the euglycemic concentration range (FIG. 13), which is why this variant was selected for structural analysis (FIG. 11).

Affinity-tuning mutations introduced in both the ttGGBP17C⋅Badan or ttGGBP182C⋅Acrylodan backgrounds yield a collection of dichromatic sensors that cover the wide range of glucose concentrations encountered in clinical chemistry.

### Example 7. Sensor Arrays for Detecting a Wide Range of Glucose Concentrations

The precision (reciprocal of the error) of individual sensor precision is maximal at the K_{d} value, and decreases at lower or higher glucose concentrations (Marvin et al. 1997 Proc Natl Acad Sci USA, 94, 4366-71). Construction of a high-precision sensor capable of spanning the entire 100-fold clinical concentration range from extreme hypoglycemia to the HHS therefore requires combining several sensors together to maintain a high precision level. In the ttGGBP182C⋅Acrylodan background, a suite of five affinity mutants can be combined that together provide high-precision coverage from 1-100 mM (FIG. 12). A suite of four mutants in the ttGGBP17C⋅Badan background provides similar coverage. It should be noted that the ttGGBP17C⋅Badan variants switch from dim green to bright blue upon binding glucose, whereas the ttGGBP182C⋅Acrylodan variants switch from bright blue to dim green. These opposing changes in color and brightness in response to glucose binding provide additional internal checks on the integrity of the observations in an array that combines sensors derived from both backgrounds.

### Example 8. Device Integration (Phase Step 5)

Protein immobilization on solid surfaces, e.g. a polymeric planar structure or bead structure, paper, glass, silica or metal, is an important step for incorporating biosensors into devices. Immobilization enables (i) spatial localization, (ii) control over the presentation of the sensors to the reader (e.g. by encoding geometries for optical readouts), (iii) selective retention in sample separation procedures. It is advantageous to control the geometry of the protein attachment to the solid surface, in order to minimize perturbation of the fluorescence sensing mechanism. Such constructs fuse an N- or C-terminal protein domain that can mediate site-specific attachment to an appropriately chemically activated surface. For instance, hexa-histidine peptide for metal-mediated immobilization, a hexa-lysine peptide for attachment to amine-reactive groups, or a zinc-finger domain (ZF-QNK) (Smith et al. 2005 Protein Sci, 14, 64-73), or a disulfide-containing truncated zinc finger (βZif)(Smith et al. 2005 Protein Sci, 14, 64-73) at N- or C-termini of the FRS to thiol-reactive groups (FIG. 13). Here we show that site-specific attachment of a robust glucose sensor to suitably derivatized agarose beads conserves its emission fluorescence spectral response, binding affinity, and thermostability.

The ttGGBP182C.2.0⋅Acrylodan protein was site-specifically immobilized through its C-terminal hexa-histidine tag on commercially available magnetic beads coated with Ni-NTA. The use of magnetic beads affords a straightforward means for holding the beads in place within their respective sensor patches in the sampling cartridge with a magnetic field. Site-specific immobilization minimizes perturbation of the sensing mechanism. Comparison of protein thermostabilities determined in solution and on beads shows that protein is stability is not perturbed by immobilization (FIG. 14A-F) These glucose-responsive magnetic beads also useful to measure highly precise glucose titration curves (FIG. 15). Similarly, hexa-lysine fusion domains were immobilized on agarose beads derivatized with N-hydroxysuccinimide, and the QNK- or βZif-fusion on iodoacetyl-derivatized beads. These fusion proteins also maintained their spectral, glucose affinities, and thermostabilities (FIG. 14D-F).

The glucose-responsive magnetic beads were dried by incubation at 50° C. for 20 minutes, using an aqueous ammonium bicarbonate buffer. The stability properties of the sensor were recovered completely upon rehydration (FIG. 14c). The dried beads were aged in situ inside fully assembled sample-handling cartridges by incubation for up to 7 days at 25 ºC, 37 ºC, and 50° C. in the dark. Fluorescence and glucose-responsive properties were tested in cartridges stored for 1, 2 and 7 days. At all temperatures, the fluorescence ratio in the absence of glucose, and the glucose affinities remained unchanged within the error of the observation. Therefore, a significant advantage of ttGGBP-based FRSs is that they are sufficiently robust to be handled at ambient temperatures in a desiccated state, greatly simplifying manufacturing, distribution, and long-term storage conditions.

### Example 9. Fluorescence Mechanism

The most effective glucose-sensing FRSs identified are based on singly labeled Acrylodan and Badan conjugates. These two dyes, and their parent, Prodan (Weber 1979 Biochemistry, 18, 3075-3078), belong to a class of fluorophores that can undergo internal rotations which change the electronic structures of excited state dipoles (Rettig 1986 Angew. Chem. Int. Ed. Engl., 25, 971-988; Grabowski et al. 2003 Chem Rev, 103, 3899-4032). The X-ray structure of Prodan shows that in the absence of external factors, the dimethyl amino (DMA) and carbonyl groups are coplanar with the naphthalene core, and the system is maximally conjugated. However, the DMA or carbonyl groups can twist out of plane, diminishing the extent of conjugation within the system, and increasing the degree of polarization of these groups. This enhanced polarization increases the magnitude of the excited state dipole, and correspondingly its sensitivity to the polarity of its surroundings, which manifests itself as general solvatochromic effects and responses to specific hydrogen-bonding interactions. There has been considerable debate regarding the magnitude of the dipole in the polarized state in Prodan (Weber 1979 Biochemistry, 18, 3075-3078; Balter 1988 Chem. Phys. Lett., 143, 565-570; Catalan 1991 Journal of Fluorescence, 1, 215-223; Nemkovich 2007 Journal of Photochemistry and Photobiology A: Chemistry, 185, 26-31; Samanta 2000 Journal of Physical Chemistry, 104, 8972-8975; Kawski 2001 Zeitschrift fur Naturforschung, 56a, 407-411; Kawski 2002 Zeitschrift fur Naturforschung, 57a, 716-722), whether the molecule undergoes twisting in the excited state, and if so, whether the carbonyl or the DMA groups twist (Nowak 1986 Journal of Molecular Structure, 139, 13-23; Heisel 1987 Chemical Physics Letters, 138, 321-326; Parusel 1997 J. Molec. Struct., 398, 341-346; Parusel 1998b Journal of Physical Chemistry, 102, 7149-7156; Parusel 1998a J. Chem. Soc., Faraday Trans., 94, 2923-2927; Mennucci 2008 Journal of Physical Chemistry, 112, 414-423; Adhikary 2009 Journal of Physical Chemistry, 113, 11999-12004; Marini 2010 Journal of Physical Chemistry, 114, 17128-17135; Pederzoli 2014 Chemical Physics Letters, 597, 57-62; Cwiklik 2011 J. Phys. Chem., 115, 11428-11437; Fukuda 2012 Chem. Phys. Lett., 552, 53-57; Barucha-Kraszewska 2010 Biochim Biophys Acta, 1798, 1724-1734; Nitschke 2012 J. Phys. Chem., 116, 2713-2721). A series of derivatives in which the DMA (Lobo 2003 Journal of Physical Chemistry, 107, 10938-10943; Davis 2005 J. Phys. Chem., 109, 1295-1298) or carbonyl (Everett 2010 Journal of Physical Chemistry, 114, 4946-4950) groups are held in planar or out-of-plane conformations have shown that it is twisting of the carbonyl and not the DMA group that alters Prodan polarization.

The collection of Acrylodan and Badan conjugates of mutant ecGGBP and ttGGBPs constructed and described herein contains several semi-synthetic glucose sensors that exhibit wavelength-dependent changes in fluorescence emission intensity in response to glucose binding. The best responses were observed for conjugates attached to ttGGBP F17C (SEQ ID NO: 51) and ttGGBP W182C (SEQ ID NO: 61) cysteines endosteric mutants that replace residues which form van der Waals contacts with the pyranose glucose ring. Remarkably, the emission intensity maxima shift in opposite directions at these two positions: glucose binding evinces a bathochromic shift for ttGGBP W182C and ecGGBP W183C Acrylodan conjugates, whereas at ttGGBP F17C and ecGGBP F16C both Acrylodan and Badan exhibit hypsochromic responses. Analysis of their ligand-mediated changes in the populations of excited state and ground state electronic transitions in combination with the X-ray structures of three different conjugates has enabled us to describe a mechanism for the fluorescence response to glucose binding.

Analysis of the emission intensity (FIGS. 16-18, Tables 7 and 8) and absorption spectra (FIG. 19) indicated that glucose binding alters the populations of two dominant electronic transitions in both the excited (S₁ and S₂) and ground (G₁ and G₂) states. In hypsochromic responses, the dominant excited state electronic transition shifts from S₁ (green) in the apo-protein, to S₂ (blue) in the glucose complex; in bathochromic responses, the opposite redistribution is observed, and the glucose complex is dominated by the S₁ (green) excited state. Similarly, in the ground state, hypsochromic responses shift the electronic transitions in the absorbance spectra from a low- (G₁) to a high-energy state (G₂); for bathochromic responses, the shift is G₂→G₁. Comparison of high-resolution X-ray structures of the ttGGBP F17C⋅Badan (hypsochromic response) and ttGGBP W182C⋅Acrylodan or ecGGBP W183C⋅Acrylodan (bathochromic responses) revealed that the fluorophore structures of the glucose complexes differ in these conjugates (FIGS. 9 and 10). From these observations we deduced that the G₁ ground state in ecGGBP W183C⋅Acrylodan corresponds to a planar fluorophore, whereas the G₂ state of F17C⋅Badan corresponds to twisted fluorophore carbonyl in which the carbonyl has repositioned out of the plane of the naphthalene ring.

These observations indicate that the changes in fluorescence intensities of glucose-responsive Acrylodan and Badan conjugates arise as a consequence of conformational coupling between ligand-mediated shifts in the population of protein conformations and internal twisting of the fluorophore carbonyl relative to its naphthalene ring. Like all periplasmic binding proteins, GGBP undergoes a large, ligand-mediated conformational change from an open to a closed state in which the ligand is enveloped between two domains that are linked by a flexible hinge. The protein conformations of the glucose complexes of the ttGGBP F17C⋅Badan, ttGGBP W182C⋅Acrylodan and ecGGBP W183C⋅Acrylodan conjugates are as closed as the unmodified, wild-type proteins. The fluorophores do not occupy the positions of the wild-type aromatic rings that they replace, but instead point outwards into the solvent such that their dimethylamino groups make no contacts with the protein and is coplanar with naphthalene ring (e.g., in the case of ttGGBP17C⋅Badan, where the structure of this group is unambiguous). By contrast, the carbonyl is located at the end of the linker through which the fluorophore is coupled to the protein cysteine. The torsion between it and the naphthalene ring therefore is affected by a combination of interactions with residues proximal to the attachment site and distal interactions with the ring. Ligand-mediated signaling occurs if two conditions are satisfied: (i) the protein interacts with both the naphthalene and the carbonyl, stabilizing their relative twist, and (ii) these interactions differ in the open and closed states.

Analysis of the spectra of all conjugates (Tables 7 and 8) showed that these signaling conditions are satisfied most commonly if the fluorophore is planar in the open, ligand-free protein conformation and twists in the closed conformation of the glucose complex. In the case of F17C⋅Badan structure, the closed protein conformation stabilizes the twisted form by interactions with both the carbonyl and the naphthalene ring. These interactions are contributed by distal residues in the domain located opposite the fluorophore attachment point, and therefore are likely to be present in the closed but not open protein conformation. Less commonly, the fluorophore twists in the open protein conformation through proximal interactions, and becomes untwisted in the closed conformation. The ttGGBP W182C⋅Acrylodan and ecGGBP W183C⋅Acrylodan conjugates represent this case. The interactions that stabilize the twisted state in the open protein conformation have not been identified, but the structure of the closed conformation reveals an absence of interactions that twist the naphthalene ring and carbonyl relative to each other and the fluorophore adopts a low-energy planar state.

Mutations that affect ligand binding (Table 6) also can influence spectroscopic properties (Table 8). In the Class 1 PCS mutant H151Q ratiometry is abolished, by diminishing the fraction of S2 that forms in the glucose complex. In D15A the redistribution of the two excited populations enhances ratiometric responses in both conjugates. In Class 2 mutants, the intrinsic equilibrium between the open and closed conformations is manipulated. In all the Class 2 mutants of both F17C Acrylodan and Badan conjugates, the fraction of the S₁ state in the apo-protein (^{apo}ƒ(S₁)) is altered. These effects can both enhance or abolish wavelength shifts (Table 7 and 8). For instance, shifts are strengthened in the mutant Badan conjugates, but abolished in the Acrylodan conjugates as evidenced by the magnitude of the C₂ component. Mutations in the carbonyl hole (Class 3a) and the fluorophore channel walls (Class 3b) have large effects on glucose binding and fluorescence spectra. Many of the N258 mutants in the carbonyl hole do not respond to glucose. The absence of shifts in thermal stability in the presence of glucose indicates that these mutants no longer bind ligand. The 17C⋅Acrylodan N258S mutant conjugate is the exception: it binds and responds to glucose, but binding does not alter the distribution of excited state populations. Mutations in residues located on either side of the channel wall strongly influence both the distributions of the excited states in the ligand-free protein, and their ligand-mediated redistribution. Mutations in A260 all switch the 17C⋅Badan conjugate from a mixture of S₁ and S₂ states in the apo-protein to a predominantly S₁ state that exhibit small redistributions in response to glucose. Accordingly, most mutant conjugates exhibit isochromic instead of hypsochromic responses to glucose, with exception of 17C⋅Badan A260W which retains (diminished) hypsochromicity. By contrast, the 17C⋅Acrylodan A260 mutants retain the mixed S₁ and S₂ population in the apo-protein, but all response to glucose binding.

Although conformational coupling between the open and closed forms of the protein and the fluorophore internal torsional equilibrium is a major factor in determining the ligand-responsive changes in fluorescence of the Acrylodan and Badan conjugates, environmental effects on excited state dipole strengths also contribute, as evidenced by the presence of fine structure in the residuals between the models and observations (FIGS. 17 and 18). The conjugates or their mutants differ in the degree to which fine structure is present in the emission spectra and responsive to glucose. This fine structure is due to changes in the energies of the S₁ and S₂ excited states associated with differences of their local environment in the open and closed conformations. The analysis presented here treats these differences as an ensemble average approximation.

The mechanism described is consistent with observations of the experimental properties of Prodan derivatives in which the twisted state of either the DMA or the carbonyl groups were controlled by synthesis of conformationally constrained groups (Lobo 2003 Journal of Physical Chemistry, 107, 10938-10943; Davis 2005 J. Phys. Chem., 109, 1295-1298; Green 2012 J. Org. Chem., 78, 1784-1789; Everett 2010 Journal of Physical Chemistry, 114, 4946-4950; Naughton 2013 J. Phys. Chem., 117, 3323-3327; Nikitina 2013 J. Phys. Chem., 117, 9189-9195; Daneri 2015 Journal of Photochemistry and Photobiology A: Chemistry, 310, 106-112).

Semisynthetic, fluorescently responsive proteins are useful in the development of reagentless biosensors with a wide variety of applications. However, in the absence of design principles that guide choice and placement of fluorophores, systematic identification of suitable fluorescent conjugates remains a significant challenge in their construction. The mechanistic insights described herein have revealed three key aspects of the fluorescence signal transduction mechanism that enable, inform, and direct rational engineering of fluorescent responses:
1. Signaling is controlled by the twisting of the carbonyl group, and changes in the specific interactions between it and the protein matrix via ligand-mediated protein conformational changes.
2. The linker length and concomitant degrees of torsional freedom determine the effectiveness of the ligand-mediated conformational coupling between the protein and the carbonyl at a given attachment position.
3. The planar ring system which determines the wavelength absorption and emission characteristics is not constrained by the protein matrix, but points outwards into the solvent.
The first two observations enable and direct structure-based approaches for identifying attachment positions and optimizing the functionalized linker for covalent modification. In such approaches, the internal flexibility of the linker is modeled, and the resulting library of fluorophore conformations are placed in a three-dimensional model of a protein host, checked for steric compatibility with their environment, and attachment sites and possible additional mutations identified that (de)stabilize a particular conformation or hydrogen bond with the protein exclusively in the apo-protein or ligand complex. The third observation indicates that the feasibility of introducing alternative chromophore structures that alter the fluorescence characteristics of the basic Prodan framework, while maintaining ligand-responsive signaling, provided the structural characteristics of the carbonyl and linker are maintained. Of particular interest are the design and construction of chromophore variants with wavelengths in the transparent window of blood (>600 nm). These principles are applied not only to periplasmic binding proteins and Prodan derivatives, but also to establish conformational coupling of any suitably placed, internally twisting fluorophore in proteins that undergo ligand-mediated changes in protein conformations.

TABLE 1

| | | | PCS position and sequence | | | |
|---|---|---|---|---|---|---|
| # | Accession code | Species | 14 | 16 | 91 | 152 |
| 1 | 2GBP (seed structure) | Escherichia coli | D | F | N | H |
| 2 | NC_013654\|YP_003350022.1 | Escherichia coli | D | F | N | H |
| 3 | NC_016822\|YP_005457115.1 | Shigella sonnei | D | F | N | H |
| 4 | NC_017328\|YP_005727882.1 | Shigella flexneri | D | F | N | H |
| 5 | NC_011740\|YP_002383354.1 | Escherichia fergusonii | D | F | N | H |
| 6 | NC_010658\|YP_001879517.1 | Shigella boydii | D | F | N | H |
| 7 | NC_022912\|Asd1617_02874 | Shigella dysenteriae | D | F | N | H |
| 8 | NC_013716\|YP_003365823.1 | Citrobacter rodentium | D | F | N | H |
| 9 | NC_009792\|YP_001452231.1 | Citrobacter koseri | D | F | N | H |
| 10 | NC_021500\|H650_06520 | Enterobacter sp. | D | F | N | H |
| 11 | NC_014121\|ECL_03459 | Enterobacter cloacae | D | F | N | H |
| 12 | NC_015968\|YP_004829427.1 | Enterobacter asburiae | D | F | N | H |
| 13 | NC_009778\|YP_001437192.1 | Cronobacter sakazakii | D | F | N | H |
| 14 | NC_016514\|YP_004952913.1 | Enterobacter cloacae | D | F | N | H |
| 15 | NC_011283\|YP_002237430.1 | Klebsiella pneumoniae | D | F | N | H |
| 16 | NC_020063\|YP_007339667.1 | Enterobacteriaceae bacterium | D | F | N | H |
| 17 | NC_016810\|YP_005182060.1 | Salmonella enterica | D | F | N | H |
| 18 | NC_013850\|YP_003438413.1 | Klebsiella variicola | D | F | N | H |
| 19 | NC_013282\|YP_003211183.1 | Cronobacter turicensis | D | F | N | H |
| 20 | NC_021232\|YP_007990486.1 | Klebsiella pneumoniae | D | F | N | H |
| 21 | NC_015761\|YP_004730836.1 | Salmonella bongori | D | F | N | H |
| 22 | NC_016612\|YP_005021097.1 | Klebsiella oxytoca | D | F | N | H |
| 23 | NC_009436\|YP_001177466.1 | Enterobacter sp. | D | F | N | H |
| 24 | NC_011147\|YP_002141464.1 | Salmonella enterica | D | F | N | H |
| 25 | NC_021066\|YP_007872566.1 | Raoultella ornithinolytica | D | F | N | H |
| 26 | NC_015663\|YP_004594960.1 | Enterobacter aerogenes | D | F | N | H |
| 27 | NC_014618\|YP_003941097.1 | Enterobacter lignolyticus | D | F | N | H |
| 28 | NC_015224\|YP_004297664.1 | Yersinia enterocolitica | D | F | N | H |
| 29 | NC_020211\|YP_007405370.1 | Serratia marcescens | D | F | N | H |
| 30 | NC_021741\|M495_07335 | Serratia liguefaciens | D | F | N | H |
| 31 | NC_014029\|YP_003567546.1 | Yersinia pestis | D | F | N | H |
| 32 | NC_010634\|YP_001872058.1 | Yersinia pseudotuberculosis | D | F | N | H |
| 33 | NC_009832\|YP_001477795.1 | Serratia proteamaculans | D | F | N | H |
| 34 | NC_015567\|YP_004504928.1 | Serratia plymuthica | D | F | N | H |
| 35 | NC_015566\|YP_004499976.1 | Serratia sp. | D | F | N | H |
| 36 | NC_015061\|YP_004212168.1 | Rahnella sp. | D | F | N | H |
| 37 | NC_016818\|YP_005199494.1 | Rahnella aquatilis | D | F | N | H |
| 38 | NC_007712\|YP_454643.1 | Sodalis glossinidius | D | F | N | H |
| 39 | NC_020418\|YP_007506508.1 | Morganella morganii | D | F | N | H |
| 40 | NC_021290\|YP_008045423.1 | Aeromonas hydrophila | D | F | N | H |
| 41 | NC_012912\|YP_003005296.1 | Dickeya zeae | D | F | N | H |
| 42 | NC_014562\|YP_003931657.1 | Pantoea vagans | D | F | N | H |
| 43 | NC_014837\|YP_004116494.1 | Pantoea sp. | D | F | N | H |
| 44 | NC_014500\|YP_003882024.1 | Dickeya dadantii | D | F | N | H |
| 45 | NC_014306\|YP_003742362.1 | Erwinia billingiae | D | F | N | H |
| 46 | NC_015424\|YP_004390764.1 | Aeromonas veronii | D | F | N | H |
| 47 | NC_013956\|YP_003520836.1 | Pantoea ananatis | D | F | N | H |
| 48 | NC_022546\|YP_008651638.1 | Plautia stali | D | F | N | H |
| 49 | NC_009348\|YP_001140155.1 | Aeromonas salmonicida | D | F | N | H |
| 50 | NC_010694\|YP_001907230.1 | Erwinia tasmaniensis | D | F | N | H |
| 51 | NC_013961\|YP_003531646.1 | Erwinia amylovora | D | F | N | H |
| 52 | NC_022268\|YP_008522161.1 | Serratia sp. | D | F | N | H |
| 53 | NC_012691\|YP_002893819.1 | Tolumonas auensis | D | F | N | H |
| 54 | NC_006371\|YP_133522.1 | Photobacterium profundum | D | F | N | H |
| 55 | NC_020802\|YP_007640992.1 | Psychromonas sp. | D | F | N | H |
| 56 | NC_022223\|N175_08855 | Listonella anguillarum | D | F | N | H |
| 57 | NC_015633\|YP_004566361.1 | Vibrio anguillarum | D | F | N | H |
| 58 | NC_016445\|YP_004936985.1 | Vibrio cholerae | D | F | N | H |
| 59 | NC_016602\|YP_004992976.1 | Vibrio furnissii | D | F | N | H |
| 60 | NC_015460\|YP_004420811.1 | Gallibacterium anatis | D | F | N | H |
| 61 | NC_014966\|YP_004190360.1 | Vibrio vulnificus | D | F | N | H |
| 62 | NC_006300\|YP_087835.1 | Mannheimia succiniciproducens | D | F | N | H |
| 63 | NC_015964\|YP_004822812.1 | Haemophilus parainfluenzae | D | F | N | H |
| 64 | NC_012913\|YP_003007706.1 | Aggregatibacter aphrophilus | D | F | N | H |
| 65 | NC_000907\|NP_438982.2 | Haemophilus influenzae | D | F | N | H |
| 66 | NC_009655\|YP_001345182.1 | Actinobacillus succinogenes | D | F | N | H |
| 67 | NC_014920\|YP_004135881.1 | Haemophilus influenza | D | F | N | H |
| 68 | NC_016513\|YP_004948894.1 | Aggregatibacter actinomycetemc | D | F | N | H |
| 69 | NC_010939\|YP_001969307.1 | Actinobacillus pleuropneumonia | D | F | N | H |
| 70 | NC_016808\|YP_005176051.1 | Pasteurella multocida | D | F | N | H |
| 71 | NC_010519\|YP_001783460.1 | Haemophilus somnus | D | F | N | H |
| 72 | NC_021082\|YP_007882660.1 | Mannheimia haemolytica | D | F | N | H |
| 73 | NC_017846\|YP_006287121.1 | Aggregatibacter actinomycetemc | D | F | N | H |
| 74 | NC_018690\|YP_006818133.1 | Actinobacillus suis | D | F | N | H |
| 75 | NC_011852\|YP_002475059.1 | Haemophilus parasuis | D | F | N | H |
| 76 | NC_008312\|YP_720691.1 | Trichodesmium erythraeum | D | F | N | H |
| 77 | NC_020515\|YP_007548075.1 | Bibersteinia trehalosi | D | F | N | H |
| 78 | NC_022524\|N288_24375 | Bacillus infantis | D | F | N | H |
| 79 | NC_021281\|YP_008019649.1 | Fusobacterium nucleatum | D | F | N | H |
| 80 | NC_003454\|NP_604062.1 | Fusobacterium nucleatum | D | F | N | H |
| 81 | NC_003366\|NP_562257.1 | Clostridium perfringens | D | F | N | H |
| 82 | NC_018607\|YP_006708369.1 | Brachyspira pilosicoli | D | F | N | H |
| 83 | NC_019908\|YP_007234804.1 | Brachyspira pilosicoli | D | F | N | H |
| 84 | NC_012225\|YP_002722709.1 | Brachyspira hyodysenteriae | D | F | N | H |
| 85 | NC_017243\|YP_005593986.1 | Brachyspira intermedia | D | F | N | H |
| 86 | NC_014150\|YP_003634598.1 | Brachyspira murdochii | D | F | N | H |
| 87 | NC_014330\|YP_003786208.1 | Brachyspira pilosicoli | D | F | N | H |
| 88 | NC_012491\|YP_002770161.1 | Brevibacillus brevis | D | F | N | H |
| 89 | NC_015732\|YP_004698509.1 | Treponema caldaria | D | F | N | H |
| 90 | NC_020291\|Cspa_c46680 | Clostridium saccharoperbutylac | D | F | N | H |
| 91 | NC_009617\|YP_001311499.1 | Clostridium beijerinckii | D | F | N | H |
| 92 | NC_014364\|YP_003802961.1 | Spirochaeta smaragdinae | D | Y | N | H |
| 93 | NC_015152\|YP_004246106.1 | Sphaerochaeta globosa | D | F | N | H |
| 94 | NC_014392\|YP_003839461.1 | Caldicellulosiruptor obsidians | D | F | N | H |
| 95 | NC_015578\|YP_004532181.1 | Treponema primitia | D | F | N | H |
| 96 | NC_014410\|YP_003852930.1 | Thermoanaerobacterium thermosa | D | F | N | H |
| 97 | NC_012914\|YP_003010600.1 | Paenibacillus sp. | D | F | N | H |
| 98 | NC_010278\|YP_001651502.1 | Actinobacillus pleuropneumonia | D | F | N | H |
| 99 | NC_016633\|YP_005062659.1 | Sphaerochaeta pleomorpha | D | F | N | H |
| 100 | NC_014393\|YP_003845273.1 | Clostridium cellulovorans | D | F | N | H |
| 101 | NC_014652\|YP_003991244.1 | Caldicellulosiruptor hydrother | D | F | N | H |
| 102 | NC_022777\|YP_008772951.1 | Clostridium tetani | D | F | N | H |
| 103 | NC_018690\|YP_006817320.1 | Actinobacillus suis | D | F | N | H |
| 104 | NC_009053\|YP_001053159.1 | Actinobacillus pleuropneumonia | D | F | N | H |
| 105 | NC_014393\|YP_003845272.1 | Clostridium cellulovorans | D | F | N | N |
| 106 | NC_010939\|YP_001968321.1 | Actinobacillus pleuropneumonia | D | F | N | H |
| 107 | NC_021658\|SCE1572_01005 | Sorangium cellulosum | N | F | N | H |
| 108 | NC_015690\|YP_004645360.1 | Paenibacillus mucilaginosus | D | F | N | H |
| 109 | NC_016935\|YP_005316341.1 | Paenibacillus mucilaginosus | D | F | N | H |
| 110 | NC_012914\|YP_003013039.1 | Paenibacillus sp. | D | F | N | H |
| 111 | NC_015519\|YP_004460241.1 | Tepidanaerobacter acetatoxydan | D | F | N | H |
| 112 | NC_013406\|YP_003243743.1 | Paenibacillus sp. | D | F | N | H |
| 113 | NC_015519\|YP_004461277.1 | Tepidanaerobacter acetatoxydan | D | F | N | H |
| 114 | NC_013517\|YP_003308491.1 | Sebaldella termitidis | D | F | N | H |
| 115 | NC_014150\|YP_003634599.1 | Brachyspira murdochii | D | F | N | H |
| 116 | NC_015436\|YP_004411990.1 | Sphaerochaeta coccoides | D | F | N | H |
| 117 | NC_021038\|YP_007827124.1 | Fretibacterium fastidiosum | D | F | N | H |
| 118 | NC_020291\|Cspa_c46620 | Clostridium saccharoperbutylac | D | F | N | H |
| 119 | NC_009617\|YP_001311493.1 | Clostridium beijerinckii | D | F | N | H |
| 120 | NC_015977\|YP_004837837.1 | Roseburia hominis | D | F | N | H |
| 121 | NC_021018\|YP_007796707.1 | Coprococcus sp. | D | F | N | H |
| 122 | NC_021042\|YP_007838599.1 | Faecalibacterium prausnitzii | D | F | N | N |
| 123 | NC_012781\|YP_002936409.1 | Eubacterium rectale | D | F | N | H |
| 124 | NC_015601\|YP_004561181.1 | Erysipelothrix rhusiopathiae | D | F | N | N |
| 125 | NC_021354\|YP_008073256.1 | Erysipelothrix rhusiopathiae | D | F | N | N |
| 126 | NC_021012\|YP_007778124.1 | Roseburia intestinalis | D | F | N | H |
| 127 | NC_022592\|CAETHG_2989 | Clostridium autoethanogenum | N | W | N | N |
| 128 | NC_014328\|CLIU_c08950 | Clostridium ljungdahlii | N | W | N | N |
| 129 | NC_021020\|YP_007799070.1 | Faecalibacterium prausnitzii | D | F | N | N |
| 130 | NC_021040\|YP_007833443.1 | Roseburia intestinalis | D | F | N | H |
| 131 | NC_021035\|YP_007824422.1 | butyrate-producing bacterium | D | F | N | N |
| 132 | NC_021012\|YP_007778116.1 | Roseburia intestinalis | D | F | N | N |
| 133 | NC_015977\|YP_004837830.1 | Roseburia hominis | D | F | N | N |
| 134 | NC_021040\|YP_007833436.1 | Roseburia intestinalis | D | F | N | N |
| 135 | NC_014376\|YP_003822569.1 | Clostridium saccharolyticum | D | F | N | N |
| 136 | NC_022549\|YP_008656214.1 | Acholeplasma brassicae | D | F | N | H |
| 137 | NC_014387\|YP_003830205.1 | Butyrivibrio proteoclasticus | D | F | N | N |
| 138 | NC_014376\|YP_003822565.1 | Clostridium saccharolyticum | D | F | N | N |

| | PCS position and sequence | | | | | | | | |
|---|---|---|---|---|---|---|---|---|---|
| # | 154 | 158 | 183 | 211 | 236 | 256 | Identity | Thermophilicity | Gram |
| 1 | D | R | W | N | D | N | | | |
| 2 | D | R | W | N | D | N | 1.00 | Mesophilic | − |
| 3 | D | R | W | N | D | N | 1.00 | Mesophilic | − |
| 4 | D | R | W | N | D | N | 1.00 | Mesophilic | − |
| 5 | D | R | W | N | D | N | 1.00 | Mesophilic | − |
| 6 | D | R | W | N | D | N | 1.00 | Mesophilic | − |
| 7 | D | R | W | N | D | N | 1.00 | Mesophilic | − |
| 8 | D | R | W | N | D | N | 0.97 | Mesophilic | − |
| 9 | D | R | W | N | D | N | 0.96 | Mesophilic | − |
| 10 | D | R | W | N | D | N | 0.95 | Mesophilic | − |
| 11 | D | R | W | N | D | N | 0.95 | Mesophilic | − |
| 12 | D | R | W | N | D | N | 0.95 | Mesophilic | − |
| 13 | D | R | W | N | D | N | 0.94 | Mesophilic | − |
| 14 | D | R | W | N | D | N | 0.94 | Mesophilic | − |
| 15 | D | R | W | N | D | N | 0.94 | Mesophilic | − |
| 16 | D | R | W | N | D | N | 0.94 | Mesophilic | + |
| 17 | D | R | W | N | D | N | 0.94 | Mesophilic | − |
| 18 | D | R | W | N | D | N | 0.94 | Mesophilic | − |
| 19 | D | R | W | N | D | N | 0.94 | Mesophilic | − |
| 20 | D | R | W | N | D | N | 0.94 | Mesophilic | − |
| 21 | D | R | W | N | D | N | 0.94 | Mesophilic | − |
| 22 | D | R | W | N | D | N | 0.94 | Mesophilic | − |
| 23 | D | R | W | N | D | N | 0.94 | Mesophilic | − |
| 24 | D | R | W | N | D | N | 0.94 | Mesophilic | − |
| 25 | D | R | W | N | D | N | 0.93 | Mesophilic | + |
| 26 | D | R | W | N | D | N | 0.93 | Mesophilic | − |
| 27 | D | R | W | N | D | N | 0.93 | Mesophilic | − |
| 28 | D | R | W | N | D | N | 0.90 | Mesophilic | − |
| 29 | D | R | W | N | D | N | 0.88 | Mesophilic | + |
| 30 | D | R | W | N | D | N | 0.88 | Mesophilic | + |
| 31 | D | R | W | N | D | N | 0.87 | Mesophilic | − |
| 32 | D | R | W | N | D | N | 0.87 | Mesophilic | − |
| 33 | D | R | W | N | D | N | 0.87 | Mesophilic | + |
| 34 | D | R | W | N | D | N | 0.86 | Mesophilic | + |
| 35 | D | R | W | N | D | N | 0.86 | ? | + |
| 36 | D | R | W | N | D | N | 0.86 | Mesophilic | + |
| 37 | D | R | W | N | D | N | 0.85 | Mesophilic | + |
| 38 | D | R | W | N | D | N | 0.84 | Mesophilic | − |
| 39 | D | R | W | N | D | N | 0.82 | Mesophilic | + |
| 40 | D | R | W | N | D | N | 0.82 | Mesophilic | − |
| 41 | D | R | W | N | D | N | 0.82 | Mesophilic | − |
| 42 | D | R | W | N | D | N | 0.81 | Mesophilic | − |
| 43 | D | R | W | N | D | N | 0.81 | Mesophilic | − |
| 44 | D | R | W | N | D | N | 0.81 | Mesophilic | − |
| 45 | D | R | W | N | D | N | 0.81 | Mesophilic | − |
| 46 | D | R | W | N | D | N | 0.81 | Mesophilic | − |
| 47 | D | R | W | N | D | N | 0.81 | Mesophilic | − |
| 48 | D | R | W | N | D | N | 0.80 | Mesophilic | + |
| 49 | D | R | W | N | D | N | 0.80 | Mesophilic | − |
| 50 | D | R | W | N | D | N | 0.80 | Mesophilic | − |
| 51 | D | R | W | N | D | N | 0.79 | Mesophilic | − |
| 52 | D | R | W | N | D | N | 0.78 | ? | + |
| 53 | D | R | W | N | D | N | 0.76 | Mesophilic | − |
| 54 | D | R | W | N | D | N | 0.74 | Psychrophilic | − |
| 55 | D | R | W | N | D | N | 0.72 | Mesophilic | + |
| 56 | D | R | W | N | D | N | 0.72 | Mesophilic | + |
| 57 | D | R | W | N | D | N | 0.72 | Mesophilic | − |
| 58 | D | R | W | N | D | N | 0.72 | Mesophilic | − |
| 59 | D | R | W | N | D | N | 0.71 | Mesophilic | − |
| 60 | D | R | W | N | D | N | 0.71 | Mesophilic | − |
| 61 | D | R | W | N | D | N | 0.71 | Mesophilic | − |
| 62 | D | R | W | N | D | N | 0.70 | Mesophilic | + |
| 63 | D | R | W | N | D | N | 0.70 | Mesophilic | − |
| 64 | D | R | W | N | D | N | 0.69 | Mesophilic | − |
| 65 | D | R | W | N | D | N | 0.69 | Mesophilic | − |
| 66 | D | R | W | N | D | N | 0.69 | Mesophilic | − |
| 67 | D | R | W | N | D | N | 0.69 | Mesophilic | − |
| 68 | D | R | W | N | D | N | 0.68 | Mesophilic | − |
| 69 | D | R | W | N | D | N | 0.68 | Mesophilic | − |
| 70 | D | R | W | N | D | N | 0.68 | Mesophilic | − |
| 71 | D | R | W | N | D | N | 0.68 | Mesophilic | − |
| 72 | D | R | W | N | D | N | 0.68 | Mesophilic | + |
| 73 | D | R | W | N | D | N | 0.68 | Mesophilic | − |
| 74 | D | R | W | N | D | N | 0.67 | Mesophilic | − |
| 75 | D | R | W | N | D | N | 0.66 | Mesophilic | − |
| 76 | D | R | W | N | D | N | 0.59 | Mesophilic | − |
| 77 | D | R | W | N | D | N | 0.58 | Mesophilic | + |
| 78 | D | R | W | N | D | N | 0.58 | Mesophilic | + |
| 79 | D | R | W | N | D | N | 0.58 | Mesophilic | − |
| 80 | D | R | W | N | D | N | 0.57 | Mesophilic | − |
| 81 | D | R | W | N | D | N | 0.56 | Mesophilic | + |
| 82 | D | R | W | N | D | N | 0.53 | Mesophilic | − |
| 83 | D | R | W | N | D | N | 0.52 | Mesophilic | − |
| 84 | D | R | W | N | D | N | 0.52 | Mesophilic | − |
| 85 | D | R | W | N | D | N | 0.52 | ? | − |
| 86 | D | R | W | N | D | N | 0.52 | Mesophilic | − |
| 87 | D | R | W | N | D | N | 0.51 | Mesophilic | − |
| 88 | D | R | W | N | D | N | 0.50 | Mesophilic | + |
| 89 | D | R | W | N | D | N | 0.50 | Mesophilic | − |
| 90 | D | R | W | N | D | N | 0.49 | Mesophilic | + |
| 91 | D | R | W | N | D | N | 0.49 | Mesophilic | + |
| 92 | D | R | W | N | D | N | 0.49 | Mesophilic | − |
| 93 | D | R | W | N | D | N | 0.48 | Mesophilic | + |
| 94 | D | R | W | N | D | N | 0.48 | Hyperthermophilic | + |
| 95 | D | R | W | N | D | N | 0.48 | Mesophilic | − |
| 96 | D | R | W | N | D | N | 0.48 | Thermophilic | + |
| 97 | D | R | W | N | D | N | 0.48 | Mesophilic | + |
| 98 | D | R | W | N | D | N | 0.48 | Mesophilic | − |
| 99 | D | R | W | N | D | N | 0.48 | Mesophilic | + |
| 100 | D | R | W | N | D | N | 0.47 | Mesophilic | + |
| 101 | D | R | W | N | D | N | 0.47 | Hyperthermophilic | + |
| 102 | D | R | W | N | D | N | 0.47 | Mesophilic | + |
| 103 | D | R | W | N | D | N | 0.47 | Mesophilic | − |
| 104 | D | R | W | N | D | N | 0.47 | Mesophilic | − |
| 105 | D | R | W | N | D | N | 0.47 | Mesophilic | + |
| 106 | D | R | W | N | D | N | 0.46 | Mesophilic | − |
| 107 | D | R | W | N | D | N | 0.46 | Mesophilic | − |
| 108 | D | R | W | N | D | N | 0.46 | Mesophilic | + |
| 109 | D | R | W | N | D | N | 0.45 | Mesophilic | + |
| 110 | D | R | W | N | D | N | 0.44 | Mesophilic | + |
| 111 | D | R | W | N | D | N | 0.44 | Mesophilic | + |
| 112 | D | R | W | N | D | N | 0.44 | Mesophilic | + |
| 113 | D | R | W | N | D | N | 0.43 | Mesophilic | + |
| 114 | D | R | W | N | D | N | 0.43 | Mesophilic | − |
| 115 | D | R | W | N | D | N | 0.43 | Mesophilic | − |
| 116 | D | R | W | N | D | N | 0.41 | Mesophilic | + |
| 117 | D | R | W | N | D | N | 0.41 | Mesophilic | + |
| 118 | D | R | W | N | D | N | 0.40 | Mesophilic | + |
| 119 | D | R | W | N | D | N | 0.39 | Mesophilic | + |
| 120 | D | R | W | N | D | N | 0.38 | Mesophilic | + |
| 121 | D | R | W | N | D | N | 0.38 | Mesophilic | + |
| 122 | D | R | W | N | D | N | 0.37 | Mesophilic | − |
| 123 | D | R | W | N | D | N | 0.37 | Mesophilic | + |
| 124 | D | R | W | N | D | N | 0.36 | Mesophilic | + |
| 125 | D | R | W | N | D | N | 0.36 | Mesophilic | + |
| 126 | D | R | W | N | D | N | 0.36 | Mesophilic | + |
| 127 | D | R | W | N | D | N | 0.36 | Mesophilic | + |
| 128 | D | R | W | N | D | N | 0.35 | Mesophilic | + |
| 129 | D | R | W | N | D | N | 0.34 | Mesophilic | − |
| 130 | D | R | W | N | D | N | 0.34 | Mesophilic | + |
| 131 | D | R | W | N | D | N | 0.33 | Mesophilic | ? |
| 132 | D | R | W | N | D | N | 0.33 | Mesophilic | + |
| 133 | D | R | W | N | D | N | 0.33 | Mesophilic | + |
| 134 | D | R | W | N | D | N | 0.32 | Mesophilic | + |
| 135 | D | R | W | N | D | N | 0.31 | Mesophilic | + |
| 136 | D | R | W | N | D | N | 0.31 | Mesophilic | + |
| 137 | D | R | W | N | D | N | 0.30 | Mesophilic | + |
| 138 | D | R | W | N | D | N | 0.29 | Mesophilic | + |

Each nucleotide and amino acid sequence associated with the accession numbers listed in Table I is hereby incorporated by reference in its entirety.

TABLE 2
Glucose-binding properties of E. coli GGBP SAFE sequence homologs.

| | | | Tₘ shift with | Percent |
|---|---|---|---|---|
| Accession number | Protein name | Expression^{a} | Glucose^{a} | Identity |
| NC_013654\|YP_003350022.1 | ecGGBP | Y | Y | 100 |
| NC_014376\|YP_003822565.1 | csaGGBP | Y | Y | 29 |
| NC_014387\|YP_003830205.1 | bprGGBP | N | | 30 |
| NC_021012\|YP_007778116.1 | rinGGBP_A | N | | 33 |
| NC_021020\|YP_007799070.1 | fprGGBP | Y | Y | 34 |
| NC_014328\|CLJU_c08950 | cljGGBP | N | | 35 |
| NC_022592\|CAETHG_2989 | cauGGBP | N | | 36 |
| NC_021012\|YP_007778124.1 | rinGGBP_B | Y | Y | 36 |
| NC_015601\|YP_004561181.1 | erhGGBP | Y | Y | 36 |
| NC_012781\|YP_002936409.1 | ereGGBP | N | | 37 |
| NC_014410\|YP_003852930.1 | ttGGBP | Y | Y | 48 |
| NC_014392\|YP_003839461.1 | cobGGBP | Y | Too | 48 |
| | | | thermostable^{b} | |
| NC_014652\|YP_003991244.1 | chyGGBP | Y | Too | 47 |
| | | | thermostable^{b} | |
| NC_013406\|YP_003243743.1 | pspGGBP | N | | 44 |

aY, Yes; N, No.
^{b}Determined using fluorescent Acrylodan conjugates (see text).

TABLE 3
Glucose response of Acrylodan and Badan conjugates
in a cysteine scan of the ttGGBP scaffold.

| | | | | | Emission | | K_{d}^{d,e} | |
|---|---|---|---|---|---|---|---|---|
| | | | | ^{apo}Tₘ | Wavelength (nm) | | (mM) | |
| Mutation | Class^{a} | Shape^{b} | Conjugate^{c} | (K) | λ1 | λ2 | ^{app}K_{d} | ^{true}K_{d} |
| Y11C | p | m | A | 351 | 511 | 470 | 0.12 | 0.16 |
| | | d | B | 349 | 492 | 528 | 0.28 | 0.24 |
| T16C | p | m | A | 351 | 519 | 460 | 8.8 | 12 |
| | | m | B | 349 | — | — | nb | nb |
| F17C | e | d | A | 349 | 482 | 542 | 0.08 | 0.06 |
| | | d | B | 346 | 467 | 491 | 0.087 | 0.26 |
| N42C | p | | A | 350 | — | — | nb | nb |
| | | | B | 324 | — | — | nb | nb |
| V67C | p | | A | 350 | — | — | nb | nb |
| | | | B | 322 | — | — | nb | nb |
| R91C | e | m | A | 349 | 491 | 540 | 0.18 | 0.17 |
| | | | B | 348 | — | — | nb^{d} | nb^{d} |
| E92C | p | | A | 350 | — | — | nb | nb |
| | | | B | 346 | — | — | nb | nb |
| A111C | p | m | A | 350 | 515 | 550 | 0.19^{d} | 0.11^{d} |
| | | m/d | B | 348 | 523 | 550 | 0.64 | 0.55 |
| Q148C | p | | A | 351 | — | — | nb | nb |
| | | | B | 349 | — | — | nb | nb |
| H151C | e | m | A | 351 | 511 | 489 | 0.012 | 0.025 |
| | | m | B | 348 | 523 | 550 | 0.018^{d} | 0.027^{d} |
| Q152C | p | | A | 351 | — | — | nb^{d} | nb^{d} |
| | | | B | 349 | — | — | nb | nb |
| N181C | p | | A | 350 | — | — | nb | nb |
| | | | B | 349 | — | — | nb | nb |
| W182C | e | d | A | 347 | 479 | 526 | 2.3 | 2.3 |
| | | m | B | 347 | 515 | 550 | 27 | 19 |
| D183C | p | | A | 348 | — | — | nb^{d} | nb^{d} |
| | | | B | 348 | — | — | nb | nb |
| L257C | a | | A | 352 | — | — | nb | nb |
| | | | B | 348 | — | — | nb | nb |
| D259C | a | | A | 349 | — | — | nb | nb |
| | | | B | 347 | — | — | nb | nb |
| K300C | a | | A | 349 | — | — | nb | nb |
| | | | B | 348 | — | — | nb | nb |

^{a}a, allosteric; e, endosteric; p, peristeric.
^{b}m, monochromatic; d, dichromatic (i.e. spectral shape change).
^{c}A, Acrylodan; B, Badan.
^{d}noisy data and or bad fit.
^{e}nb; no binding, nd; not determined.

TABLE 4
Responses of fluorophores conjugated to F17C and W182C mutants of ttGGBP^{a}.

| | | λₑₓ | ^{apo}λₘₐₓ | ^{apo}Iₘₐₓ | ^{sat}λₘₐₓ | ^{sat}Iₘₐₓ | ^{true}K_{d} |
|---|---|---|---|---|---|---|---|
| Position | Fluorophore^{b} | (nm)^{c} | (nm) | (AU ×1000) | (nm) | (AU ×1000) | (mM) |
| 17C | Acrylodan | 391 | 487 | 15.9 | 487 | 20.0 | 0.2 |
| | Badan | 391 | 519 | 12.8 | 467 | 52.6 | 0.1 |
| | 5-IAF | 491 | 523 | 64.3 | 523 | 70.6 | 61.3 |
| | Oregon green | 496 | 523 | 101.9 | 523 | 91.7 | — |
| | CPM | 384 | 471 | 78.5 | 467 | 90.9 | 17.0 |
| | IANBD | 478 | 531 | 15.3 | 535 | 19.8 | 11.8 |
| | IAEDANS | 336 | 467 | 12.5 | 467 | 14.4 | — |
| | Pacific Blue | 410 | 451 | 28.6 | 455 | 108.6 | 60.7 |
| 182C | Acrylodan | 391 | 479 | 55.0 | 515 | 17.2 | 6.0 |
| | Badan | 391 | 515 | 16.8 | 515 | 11.9 | 64.9 |
| | 5-IAF | 491 | 519 | 255.0 | 519 | 453.2 | 5.0 |
| | 6-IAF | 491 | 513 | 50.4 | 513 | 63.1 | 750 |
| | Oregon green | 496 | 519 | 78.2 | 519 | 186.2 | 20.0 |
| | CPM | 384 | 479 | 49.5 | 483 | 40.3 | 6.8 |
| | IANBD | 478 | 543 | 29.1 | 547 | 12.5 | 210 |
| | IAEDANS | 336 | 487 | 3.7 | 483 | 7.9 | 0.2 |
| | Pacific Blue | 410 | 455 | 115.0 | 455 | 119.4 | — |
| | BODIPY 499 | 499 | 519 | 40.1 | 515 | 80.2 | 30.7 |
| | BODIPY 507 | 507 | 531 | 32.9 | 535 | 31.4 | 11.0 |
| | Alexa 488 | 495 | 519 | 211.6 | 519 | 182.2 | — |
| | Alexa 532 | 532 | 551 | 63.0 | 551 | 61.0 | — |
| | Alexa 546 | 546 | 571 | 152.1 | 571 | 150.2 | — |
| | Texas Red | 595 | 611 | 23.3 | 611 | 23.5 | — |
| | Cy 5 | 646 | 663 | 19.2 | 663 | 23.3 | 210 |
| | PyPMPO | 415 | 555 | 3.7 | 559 | 4.2 | 20.6 |

^{a}λₑₓ, preferred excitation wavelength (from supplier); ^{apo}λₘₐₓ, observed maximum emission wavelength of the apo-protein; ^{apo}Iₘₐₓ, observed intensity at ^{apo}λₘₐₓ; ^{apo}λₘₐₓ, observed maximum emission wavelength of the glucose complex; ^{sat}Iₘₐₓ, observed intensity at ^{sat}λₘₐₓ; ^{true}K_{d}, affinity determined from fit of equation 1 to the monochromatic emission intensities. Emission spectra were measured on the Nanodrop3300, using ~10 μM protein. The observed absolute emission intensities are a rough guide to the brightness of the conjugate, because the protein concentration was approximately the same for each experiment.
bAbbreviations, chemical names and supplier catalogue numbers as follows: Acrylodan (A433); Badan (B6057); 5-IAF (I30451); Oregon Green 488 (O6034); CPM (D346); IANBD (D2004); IAEDANS (I14); Pacific Blue (P30506); BODIPY 499 (D20350); BODIPY 507 (D6004); BODIPY 577 (D20351); Alexa 532 (A10255); Alexa 555 (A20346); Texas Red (T6008); PyMPO (M6026) from Life Technologies and Cy5 (13080) from Lumiprobe.
^{c}The Nanodrop3300 fixed wavelength LED that most closely matched λₑₓ was used (see Materials and Methods).

TABLE 5
Crystallographic data collection and refinement statistics.^{a}

| | ttGGBP17C- | ttGGBP182C.2.0- | ecGGBP183C- |
|---|---|---|---|
| | Badan | Acrylodan | Acrylodan |
| X-ray source | 22-ID, SER-CAT, | 22-ID, SER-CAT, | 22-BM, SER-CAT, |
| | APS | APS | APS |
| Wavelength (Å) | 1.0 | 1.0 | 1.0 |
| Space Group | P2₁2₁2₁ | P2₁2₁2₁ | C121 |
| Unit Cell parameters (Å) | | | |
| a, b, c | 45.49, 53.06, 134.32 | 45.67, 53.17, | 119.58, 36.53, |
| | | 133.57 | 79.90 |
| | | | 90.00 (α), 124.13 |
| | | | (β), 90.00 (γ) |
| Resolution range (Å) | 41.63-1.59 (1.62-1.59) | 33.70-1.39 | 50.00-1.53 (1.56-1.53) |
| Completeness (%) | 99.8 (99.3) | 98.9 (94.9) | 98.8 (94.6) |
| No. of unique reflections | 44590 | 65108 (9969) | 43030 |
| Wilson B-factor (Å²) | 15.50 | 11.50 | 12.60 |
| Multiplicity | 7.21 (7.22) | 5.29 (5.17) | 6.5 (5.1) |
| R-sym (%) | 0.08 (0.69) | 0.08 (0.76) | 0.06 (0.29) |
| R-pim (%) | 0.03 (0.25) | 0.04 (0.34) | 0.025 (0.131) |
| Mean I/σ (I) | 16.40 (3.58) | 13.71 (2.87) | 25.54 (3.99) |
| Refinement statistics | | | |
| R factor (%) | 15.11 | 15.88 | 15.04 |
| Free_R_factor (%) | 17.07 | 17.74 | 16.75 |
| Average B-factor (Å²) | 20.10 | 17.90 | 18.60 |
| Macromolecules | 17.70 | 15.30 | 15.90 |
| Water | 34.30 | 34.10 | 34.60 |
| Glucose/Fluorophore | 9.50/39.90 | 8.60/39.10 | 22.70/64.50 |
| Number of non-hydrogen | | | |
| atoms | | | |
| Macromolecule | 2471 | 2493 | 2358 |
| Glucose | 24^{b} | 24^{b} | 72^{c} |
| Calcium | 1 | 1 | 1 |
| Fluorophore | 16 | 17 | 17 |
| Water | 407 | 391 | 325 |
| RMS deviations | | | |
| RMS (bonds) | 0.007 | 0.005 | 0.006 |
| RMS (angles) | 1.112 | 0.822 | 1.060 |
| Ramachandran favoured (%) | 97.81 | 98.44 | 98.71 |
| Ramachandran allowed (%) | 1.88 | 1.25 | 0.97 |
| Ramachandran outliers (%) | 0.31 | 0.31 | 0.32 |
| Rotamer outliers (%) | 0.00 | 0.00 | 0.00 |
| Clashscore | 1.60 | 0.79 | 1.03 |

^{a}Values for highest resolution shell are given in parentheses.
^{b}Two conformations.
^{c}Five molecules.

TABLE 6
Responses of mutant HGGBP17C and ttGGBP182C conjugates^{a}.

| | | | | | Emission | | Glucose^{d} | | Galactose^{d} | |
|---|---|---|---|---|---|---|---|---|---|---|
| | | | | ^{apo}Tₘ | wavelength (nm) | | ^{app}K_{d} | ^{true}K_{d} | ^{true}K_{d} | |
| Protein | Mutation | Class^{b} | Conjugate^{c} | (K) | λ1 | λ2 | (mM) | (mM) | (mM) | S |
| ttGBP17C | | | B | 346 | 467 | 519 | 0.10 | 0.15 | 0.19 | 1.3 |
| ″ | | | A | 349 | 487 | 515 | 0.08 | 0.09 | 3.8 | 43 |
| ttGGBP17C.1 | R91K, Q148E | 2 | B | | 463 | 515 | 0.6 | 0.8 | 0.4 | 0.46 |
| ttGGBP17C.2 | R69P, Q152P | 2 | B | 350 | 479 | 523 | 8.6 | 10.8 | 5.2 | 0.48 |
| ″ | ″ | 2 | A | | 492 | 515 | 15 | 18 | 5.0 | 0.28 |
| ttGGBP17C.3 | T16N, D211A | 2 | B | 346 | 471 | 531 | 3.4 | 4.1 | 2.5 | 0.61 |
| ″ | ″ | 2 | A | | 495 | 529 | 1.4 | 1.5 | 0.53 | 0.35 |
| ttGGBP17C.4 | H151Q | 1 | B | 347 | 511 | 457 | 16 | 6.4 | 14 | 2.2 |
| ″ | ″ | 1 | A | | 488 | 550 | 50 | 48 | 14 | 0.29 |
| ttGGBP17C.5 | D15A | 1 | B | 345 | 487 | 530 | 16 | 16 | 4.2 | 0.26 |
| ″ | ″ | 1 | A | 348 | 483 | 498 | 6 | 6 | 2.4 | 0.41 |
| ttGGBP17C.6 | D15E | 1 | B | 346 | 467 | 525 | 3.3 | 3.6 | 3.6^{e} | 1 |
| ttGGBP17C.7 | D15N | 1 | B | 347 | 483 | 515 | 0.75 | 1.0 | 0.64 | 0.64 |
| ″ | ″ | 1 | A | 348 | 483 | 515 | 0.3 | 0.3 | 0.3 | 0.91 |
| ttGGBP17C.8 | T16N | 2 | A | 348 | 487 | 529 | 0.61 | 0.60 | 0.26 | 0.43 |
| ttGGBP17C.9 | T16S | 2 | A | | 487 | 520 | 0.2 | 0.20 | nd | |
| ttGGBP17C.10 | G20A | 3 | A | 351 | 487 | 520 | 0.4 | 0.4 | nd | |
| ttGGBP17C.11 | T240A | 3 | A | 348 | 487 | 500 | 0.04 | 0.04 | nd | |
| ttGGBP17C.19 | N258D | 3 | B | 344 | | | nb^{e} | nb^{e} | nb^{e} | |
| ttGGBP17C.20 | N258S | 3 | B | 344 | | | nb^{e} | nb^{e} | nb^{e} | |
| ttGGBP17C.21 | N258A | 3 | B | 345 | 532 | 494 | 61 | 69 | nb | |
| ttGGBP17C.22 | A260N | 3 | B | 343 | 523 | 492 | 12 | 15 | 530^{e} | |
| ttGGBP17C.23 | A260Q | 3 | B | 344 | 527 | 490 | 15 | 18 | nb^{c} | |
| ttGGBP17C.24 | A260R | 3 | B | 344 | 490 | 515 | 1.8 | 1.7 | 29 | 17 |
| ttGGBP17C.25 | A260K | 3 | B | | 515 | 493 | 3.2 | 3.7 | nb^{e} | |
| ttGGBP17C.26 | A260W | 3 | B | 346 | 523 | 509 | 0.9 | 0.9 | 14 | 16 |
| ttGGBP17C.27 | A260F | 3 | B | 346 | 523 | 490 | 0.2 | 0.3 | 8.8 | 33 |
| ttGGBP17C.28 | A260Y | 3 | B | 346 | 523 | 494 | 0.06 | 0.07 | 2.6 | 37 |
| ttGGBP17C.29 | A260S | 3 | B | 343 | 527 | 496 | 2.1 | 2.2 | 250 | 114 |
| ttGGBP182C | | | A | 347 | 472 | 535 | 2.2 | 2.3 | 3.3 | 1.4 |
| ttGGBP182C.2.0^{f} | R91K Q148E | 2, 3 | A | 346 | 475 | 545 | 4.5 | 6.0 | 18.5 | 3.1 |
| ttGGBP182C.2.1^{g} | A154S | 1 | A | 328 | 480 | 552 | 3.0 | 4.1 | 13.1 | 3.2 |
| ttGGBP182C.2.3^{g} | A154N | 1 | A | 346 | 470 | 537 | 16.3 | 19.0 | 207 | 10.9 |
| ttGGBP182C.2.4^{g} | A154M | 1 | A | 346 | 472 | 540 | 0.4 | 0.4 | 4.5 | 11.2 |
| ttGGBP182C.2.5^{g} | H151Q | 1 | A | 346 | 477 | 542 | 10.8 | 13.0 | 20.3 | 1.6 |
| ttGGBP182C.2.6^{g} | H151N | 1 | A | 347 | 475 | 537 | 19.2 | 17.9 | 52.6 | 2.9 |
| ttGGBP182C.2.7^{g} | H151F | 1 | A | 346 | 475 | 545 | 121 | 124 | | |
| ttGGBP182C.2.8^{g} | D15N | 1 | A | 343 | 474 | 542 | 179 | 209 | 372 | 1.8 |
| ttGGBP182C.2.9^{g} | A154F | 1 | A | 345 | 512 | 477 | 0.4 | 0.3 | 1.6 | 4.0 |
| ttGGBP182C.3^{f} | R91K | 2 | A | 346 | 494 | 459 | 1.8 | 0.9 | 3.1 | 3.4 |
| ttGGBP182C.4^{f} | Q148E | 2 | A | 347 | 475 | 542 | 0.6 | 0.8 | 6.5 | 8.1 |
| ttGGBP182C.5^{f} | R69P Q152P | 2 | A | 349 | 477 | 535 | 7.5 | 8.8 | 7.4 | 0.8 |
| ttGGBP182C.6^{f} | T16N D211A | 2 | A | 345 | 472 | 530 | 21.6 | 27.6 | 81 | 2.9 |
| ttGGBP182C.7^{f} | R91K Q148S | 2, 3 | A | 346 | 478 | 550 | 0.3 | 0.4 | 1.1 | 3.7 |
| ttGGBP182C.8^{f} | R91K Q148K | 2, 3 | A | 346 | 475 | 545 | 28.7 | 44.2 | 256 | 5.8 |
| ttGGBP182C.9^{f} | D15N | 1 | A | 343 | 475 | 540 | 75.1 | 76.4 | 282 | 3.7 |

^{a}Measured on the Nanodrop at room temperature. λₘₐₓ is the wavelength corresponding to the maximum emission intensity. Optimal ratiometry wavelengths are determined according to the analysis described in Materials and Methods (equation 7). The ^{true}K_{d} is determined from monochromatic titration curves; ^{app}K_{d} from dichromatic ratiometry (equations 5 and 6). Average relative error in the ^{true}K_{d} values is 5%, in the ^{app}K_{d} values, 1%. S is the selectivity between glucose and galactose, S = ^{true}K_{d}(galactose)/^{true}K_{d}(glucose); S > 1, selective for glucose.
^{b}1, PCS; 2, inter-domain interaction; 3, contact between protein and fluorophore
^{c}A, Acrylodan; B, Badan.
^{d}nb, no bonding; nd, not determined.
^{e}Noisy data or bad fit.
^{f}Additional mutation constructed in ttGGBP182C.
^{g}Additional mutation constructed in ttGGBP182C.2.0.

TABLE 7
Spectral analysis of ttGGBP and ecGGBP
Acrylodan and Badan conjugates^{a}.

| | | | SVD component | | Gaussians | | | |
|---|---|---|---|---|---|---|---|---|
| Muta- | | | fractions | | Peaks (nm) | | Fraction f(S₁) | |
| tion | Conjugate^{b} | | C₁ | C₂ | S₁ | S₂ | Apo | Sat |
| Y11C | A | r (h) | 0.97 | 0.03 | 515 | 463 | 1.00 | 0.88 |
| | B | r (h) | 0.84 | 0.16 | 532 | 484 | 1.00 | 0.47 |
| T16C | A | n | — | — | 518 | — | 1.00 | — |
| | B | n | — | — | 539 | 446 | 0.96 | — |
| F17C | A | r (h) | 0.90 | 0.09 | 527 | 486 | 0.56 | 0.16 |
| | B | r (h) | 0.89 | 0.10 | 535 | 474 | 0.66 | 0.15 |
| F16C | A | r (h) | 0.91 | 0.08 | 509 | 454 | 0.63 | 0.10 |
| (Ec) | | | | | | | | |
| | B | r (h) | 0.92 | 0.06 | 522 | 469 | 0.56 | 0.14 |
| N42C | A | n | — | — | 518 | — | 1.00 | — |
| | B | n | — | — | 516 | — | 1.00 | — |
| V67C | A | n | — | — | 522 | 464 | 0.59 | — |
| | B | n | — | — | 523 | 480 | 0.38 | — |
| R91C | A | i | 0.97 | 0.02 | 533 | 488 | 0.43 | 0.92 |
| | B | n | — | — | 522 | 476 | 0.51 | — |
| E92C | A | n | — | — | 520 | — | 1.00 | — |
| | B | n | — | — | 522 | — | 1.00 | — |
| A111C | A | d | 0.98 | 0.01 | 520 | 474 | 0.98 | 0.95 |
| | B | d | 0.98 | 0.02 | 529 | 467 | 0.91 | 0.84 |
| Q148C | A | n | — | — | 537 | 491 | 0.26 | — |
| | B | n | — | — | 576 | 519 | 0.15 | — |
| H151C | A | i | 0.97 | 0.02 | 520 | 490 | 1.00 | 0.86 |
| | B | i | 0.98 | 0.01 | 533 | 477 | 0.98 | 1.00 |
| Q152C | A | n | — | — | 520 | — | 1.00 | — |
| | B | n | — | — | 524 | 472 | 0.74 | — |
| N181C | A | n | — | — | 527 | — | 1.00 | — |
| | B | n | — | — | 545 | — | 1.00 | — |
| W182C | A | r (b) | 0.87 | 0.13 | 500 | 463 | 0.63 | 0.99 |
| | B | d | 0.98 | 0.02 | 521 | 479 | 0.91 | 1.00 |
| W183C | A | r (b) | 0.84 | 0.15 | 527 | 481 | 0.37 | 0.81 |
| (Ec) | | | | | | | | |
| D183C | A | d | | | 523 | — | 1.00 | — |
| | B | n | — | — | 530 | 477 | 0.61 | — |
| L257C | A | n | — | — | 533 | 496 | 0.29 | — |
| | B | n | — | — | 516 | — | 1.00 | — |
| D259C | A | n | — | — | 516 | — | 1.00 | — |
| | B | n | — | — | 521 | — | 1.00 | — |
| K300C | A | n | — | — | 520 | — | 1.00 | — |
| | B | n | — | — | 528 | — | 1.00 | — |

^{a}Emission spectra determined on Nanodrop3300. SVD analysis according to equations 11-13. Gaussian fits according to equations 14-15.
^{b}A, Acrylodan; B, Badan; n, no observed response to glucose; r (h), ratiometric (hypsochromic shift); r (b), ratiometric (bathochromic shift); i, intensity increase only; d, intensity decrease only.

TABLE 8
Spectral analysis of ttGGBP.17C Acrylodan and Badan conjugates^{a}.

| | | | | SVD component | | Gaussians | | | |
|---|---|---|---|---|---|---|---|---|---|
| | | | | fractions | | Peaks (nm) | | Fraction f(S₁) | |
| Protein | Mutation | Conjugate^{b} | | C₁ | C₂ | S₁ | S₂ | Apo | Sat |
| ttGGBP17C.1 | R91K, Q148E | A | d | 0.95 | 0.04 | 518 | 490 | 0.71 | 0.60 |
| ″ | ″ | B | r(h) | 0.85 | 0.14 | 532 | 472 | 0.64 | 0.15 |
| ttGGBP17C.2 | R69P, Q152P | A | d | 0.92 | 0.06 | 528 | 462 | 0.88 | 0.73 |
| ″ | ″ | B | r(h) | 0.86 | 0.13 | 542 | 476 | 0.64 | 0.36 |
| ttGGBP17C.3 | T16N, D211A | A | d | 0.94 | 0.05 | 519 | 458 | 0.83 | 0.70 |
| ″ | ″ | B | r(h) | 0.81 | 0.18 | 540 | 474 | 0.70 | 0.30 |
| ttGGBP17C.8 | T16N | A | r(h) | 0.93 | 0.07 | 533 | 496 | 0.30 | 0.00 |
| ttGGBP17C.9 | T16S | A | r(h) | 0.94 | 0.06 | 510 | 488 | 1.00 | 0.43 |
| ttGGBP17C.4 | H151Q | A | r(h) | 0.95 | 0.04 | 526 | 485 | 0.65 | 0.52 |
| ″ | ″ | B | r(h) | 0.95 | 0.04 | 540 | 486 | 0.50 | 0.39 |
| ttGGBP17C.5 | D15A | A | r(h) | 0.87 | 0.13 | 491 | 465 | 0.98 | 0.22 |
| ″ | ″ | B | r(h) | 0.84 | 0.15 | 525 | 470 | 0.58 | 0.16 |
| ttGGBP17C.6 | D15E | A | n | — | — | 511 | 448 | 0.72 | — |
| ″ | ″ | B | r(h) | 0.90 | 0.09 | 525 | 473 | 0.39 | 0.15 |
| ttGGBP17C.7 | D15N | A | d | 0.97 | 0.02 | 492 | 464 | 0.86 | 1.00 |
| ″ | ″ | B | d | 0.92 | 0.05 | 533 | 480 | 0.22 | 0.16 |
| ttGGBP17C.19 | N258D | A | n | — | — | 525 | 475 | 0.30 | — |
| ″ | ″ | B | n | — | — | 523 | — | 1.00 | — |
| ttGGBP17C.20 | N258S | A | d | 0.99 | 0.00 | 530 | 479 | 0.37 | 0.36 |
| ″ | ″ | B | n | — | — | 524 | — | 1.00 | — |
| ttGGBP17C.21 | N258A | A | n | — | — | 526 | 475 | 0.33 | — |
| ″ | ″ | B | n | — | — | 523 | — | 1.00 | — |
| ttGGBP17C.10 | G20A | A | d | 0.95 | 0.04 | 534 | 487 | 0.28 | 0.21 |
| ttGGBP17C.11 | T240A | A | d | 0.95 | 0.03 | 541 | 487 | 0.35 | 0.25 |
| ttGGBP17C.22 | A260N | A | n | — | — | 528 | 475 | 0.33 | — |
| ″ | ″ | B | i | 0.97 | 0.03 | 531 | 480 | 0.96 | 0.85 |
| ttGGBP17C.23 | A260Q | A | i | — | — | 526 | 474 | 0.31 | — |
| ″ | ″ | B | i | 0.97 | 0.03 | 532 | 475 | 0.95 | 0.85 |
| ttGGBP17C.24 | A260R | A | n | — | — | 525 | 474 | 0.26 | — |
| ″ | ″ | B | i | 0.96 | 0.03 | 523 | 483 | 0.90 | 0.75 |
| ttGGBP17C.25 | A260K | A | n | — | — | 526 | 473 | 0.24 | — |
| ″ | ″ | B | i | 0.96 | 0.03 | 519 | 482 | 0.90 | 0.76 |
| ttGGBP17C.26 | A260W | A | i | 0.99 | 0.01 | 530 | 479 | 0.42 | 0.41 |
| ″ | ″ | B | r(h) | 0.96 | 0.04 | 529 | 478 | 1.00 | 0.87 |
| ttGGBP17C.27 | A260F | A | n | — | — | 523 | 477 | 0.38 | — |
| ″ | ″ | B | i | 0.96 | 0.04 | 529 | 487 | 1.00 | 0.82 |
| ttGGBP17C.28 | A260Y | A | n | — | — | 523 | 477 | 0.38 | — |
| ″ | ″ | B | i | 0.97 | 0.03 | 530 | 480 | 1.00 | 0.87 |
| ttGGBP17C.29 | A260S | A | n | — | — | 526 | 474 | 0.32 | — |
| ″ | ″ | B | i | 0.96 | 0.03 | 533 | 478 | 0.95 | 0.83 |

^{a}Emission spectra determined on Nanodrop3300. SVD analysis according to equations 11-13. Gaussian fits according to equations 14-15.
^{b}A, Acrylodan; B, Badan; n, no observed response to glucose; r(h), ratiometric (hypsochromic shift); r(b), ratiometric (bathochromic shift); i, intensity increase only; d, intensity decrease only.

### Example 10. Crystal Structure Coordinates for an E. coli Glucose-Galactose Binding Protein: ECGGBP183C (Acrylodan Attached to W183C Mutant)

Naming is standard three-letter amino acid code.

Atom positions are provided as Cartesian coordinates, using standard Protein Databank (PDB) format. ATOM records refer to amino acid atoms; HETATM records refer to non-amino acid atoms.

Column 1: record type (ATOM or HETATM); column 2: atom number; column 3: atom name (standard naming scheme for amino acids); column 4: residue name (ATOM records), or component name (HETATM records); column 5: chain identifier (A, B, C, . . . ); column 6: amino acid residue sequence number (ATOM records), or component number (HETATM records); columns 7-9: x,y,z atomic Cartesian positional coordinates; column 10: fractional occupancy (set to 1.0 in this listing); column 11: B-factor (ignored in this listing); column 12: file identifier (ignored in this listing); column 13: line number (same as atom number in this listing).

For heteroatom (HETATM) records, the component name (column 4) is as follows:

CA, calcium
HOH, Water
ACR, Acrylodan
K, potassium
EDO, ethylene glycol

| | | | | | | | | | | | | |
|---|---|---|---|---|---|---|---|---|---|---|---|---|
| ATOM | 1 | O | ALA | A | 2 | 106.533 | −15.459 | 75.201 | 1.00 | 0.00 | xxxx | 1 |
| ATOM | 2 | N | ALA | A | 2 | 107.059 | −18.172 | 74.462 | 1.00 | 0.00 | xxxx | 2 |
| ATOM | 3 | CA | ALA | A | 2 | 105.803 | −17.753 | 75.070 | 1.00 | 0.00 | xxxx | 3 |
| ATOM | 4 | C | ALA | A | 2 | 105.616 | −16.243 | 74.951 | 1.00 | 0.00 | xxxx | 4 |
| ATOM | 5 | CB | ALA | A | 2 | 105.744 | −18.181 | 76.527 | 1.00 | 0.00 | xxxx | 5 |
| ATOM | 6 | N | THR | A | 3 | 104.414 | −15.847 | 74.566 | 1.00 | 0.00 | xxxx | 6 |
| ATOM | 7 | CA | THR | A | 3 | 104.079 | −14.441 | 74.397 | 1.00 | 0.00 | xxxx | 7 |
| ATOM | 8 | C | THR | A | 3 | 103.547 | −13.847 | 75.689 | 1.00 | 0.00 | xxxx | 8 |
| ATOM | 9 | O | THR | A | 3 | 102.489 | −14.249 | 76.156 | 1.00 | 0.00 | xxxx | 9 |
| ATOM | 10 | CB | THR | A | 3 | 103.023 | −14.268 | 73.301 | 1.00 | 0.00 | xxxx | 10 |
| ATOM | 11 | OG1 | THR | A | 3 | 103.556 | −14.752 | 72.060 | 1.00 | 0.00 | xxxx | 11 |
| ATOM | 12 | CG2 | THR | A | 3 | 102.616 | −12.817 | 73.168 | 1.00 | 0.00 | xxxx | 12 |
| ATOM | 13 | N | ARG | A | 4 | 104.268 | −12.892 | 76.272 | 1.00 | 0.00 | xxxx | 13 |
| ATOM | 14 | CA | ARG | A | 4 | 103.781 | −12.274 | 77.501 | 1.00 | 0.00 | xxxx | 14 |
| ATOM | 15 | C | ARG | A | 4 | 102.892 | −11.082 | 77.169 | 1.00 | 0.00 | xxxx | 15 |
| ATOM | 16 | O | ARG | A | 4 | 103.286 | −10.224 | 76.378 | 1.00 | 0.00 | xxxx | 16 |
| ATOM | 17 | CB | ARG | A | 4 | 104.947 | −11.827 | 78.392 | 1.00 | 0.00 | xxxx | 17 |
| ATOM | 18 | CG | ARG | A | 4 | 105.839 | −12.971 | 78.910 | 1.00 | 0.00 | xxxx | 18 |
| ATOM | 19 | CD | ARG | A | 4 | 106.999 | −12.408 | 79.742 | 1.00 | 0.00 | xxxx | 19 |
| ATOM | 20 | NE | ARG | A | 4 | 107.840 | −13.464 | 80.300 | 1.00 | 0.00 | xxxx | 20 |
| ATOM | 21 | CZ | ARG | A | 4 | 108.891 | −13.259 | 81.091 | 1.00 | 0.00 | xxxx | 21 |
| ATOM | 22 | NH1 | ARG | A | 4 | 109.247 | −12.027 | 81.435 | 1.00 | 0.00 | xxxx | 22 |
| ATOM | 23 | NH2 | ARG | A | 4 | 109.589 | −14.293 | 81.541 | 1.00 | 0.00 | xxxx | 23 |
| ATOM | 24 | N | ILE | A | 5 | 101.699 | −11.040 | 77.760 | 1.00 | 0.00 | xxxx | 24 |
| ATOM | 25 | CA | ILE | A | 5 | 100.798 | −9.892 | 77.638 | 1.00 | 0.00 | xxxx | 25 |
| ATOM | 26 | C | ILE | A | 5 | 100.536 | −9.303 | 79.016 | 1.00 | 0.00 | xxxx | 26 |
| ATOM | 27 | O | ILE | A | 5 | 100.148 | −10.025 | 79.938 | 1.00 | 0.00 | xxxx | 27 |
| ATOM | 28 | CB | ILE | A | 5 | 99.484 | −10.290 | 76.931 | 1.00 | 0.00 | xxxx | 28 |
| ATOM | 29 | CG2 | ILE | A | 5 | 98.491 | −9.115 | 76.932 | 1.00 | 0.00 | xxxx | 29 |
| ATOM | 30 | CG1 | ILE | A | 5 | 99.787 | −10.790 | 75.514 | 1.00 | 0.00 | xxxx | 30 |
| ATOM | 31 | CD1 | ILE | A | 5 | 98.552 | −11.286 | 74.772 | 1.00 | 0.00 | xxxx | 31 |
| ATOM | 32 | N | GLY | A | 6 | 100.775 | −8.001 | 79.158 | 1.00 | 0.00 | xxxx | 32 |
| ATOM | 33 | CA | GLY | A | 6 | 100.613 | −7.324 | 80.436 | 1.00 | 0.00 | xxxx | 33 |
| ATOM | 34 | C | GLY | A | 6 | 99.242 | −6.689 | 80.534 | 1.00 | 0.00 | xxxx | 34 |
| ATOM | 35 | O | GLY | A | 6 | 98.785 | −6.074 | 79.579 | 1.00 | 0.00 | xxxx | 35 |
| ATOM | 36 | N | VAL | A | 7 | 98.583 | −6.847 | 81.678 | 1.00 | 0.00 | xxxx | 36 |
| ATOM | 37 | CA | VAL | A | 7 | 97.232 | −6.313 | 81.871 | 1.00 | 0.00 | xxxx | 37 |
| ATOM | 38 | C | VAL | A | 7 | 97.186 | −5.551 | 83.186 | 1.00 | 0.00 | xxxx | 38 |
| ATOM | 39 | O | VAL | A | 7 | 97.627 | −6.074 | 84.212 | 1.00 | 0.00 | xxxx | 39 |
| ATOM | 40 | CB | VAL | A | 7 | 96.160 | −7.449 | 81.885 | 1.00 | 0.00 | xxxx | 40 |
| ATOM | 41 | CG1 | VAL | A | 7 | 94.781 | −6.891 | 82.114 | 1.00 | 0.00 | xxxx | 41 |
| ATOM | 42 | CG2 | VAL | A | 7 | 96.199 | −8.257 | 80.605 | 1.00 | 0.00 | xxxx | 42 |
| ATOM | 43 | N | THR | A | 8 | 96.645 | −4.333 | 83.183 | 1.00 | 0.00 | xxxx | 43 |
| ATOM | 44 | CA | THR | A | 8 | 96.381 | −3.648 | 84.448 | 1.00 | 0.00 | xxxx | 44 |
| ATOM | 45 | C | THR | A | 8 | 94.872 | −3.426 | 84.569 | 1.00 | 0.00 | xxxx | 45 |
| ATOM | 46 | O | THR | A | 8 | 94.217 | −2.977 | 83.620 | 1.00 | 0.00 | xxxx | 46 |
| ATOM | 47 | CB | THR | A | 8 | 97.147 | −2.294 | 84.570 | 1.00 | 0.00 | xxxx | 47 |
| ATOM | 48 | OG1 | THR | A | 8 | 96.693 | −1.384 | 83.556 | 1.00 | 0.00 | xxxx | 48 |
| ATOM | 49 | CG2 | THR | A | 8 | 98.676 | −2.510 | 84.446 | 1.00 | 0.00 | xxxx | 49 |
| ATOM | 50 | N | ILE | A | 9 | 94.335 | −3.761 | 85.739 | 1.00 | 0.00 | xxxx | 50 |
| ATOM | 51 | CA | ILE | A | 9 | 92.933 | −3.546 | 86.095 | 1.00 | 0.00 | xxxx | 51 |
| ATOM | 52 | C | ILE | A | 9 | 92.893 | −2.377 | 87.059 | 1.00 | 0.00 | xxxx | 52 |
| ATOM | 53 | O | ILE | A | 9 | 93.694 | −2.340 | 87.986 | 1.00 | 0.00 | xxxx | 53 |
| ATOM | 54 | CB | ILE | A | 9 | 92.334 | −4.809 | 86.743 | 1.00 | 0.00 | xxxx | 54 |
| ATOM | 55 | CG1 | ILE | A | 9 | 92.474 | −6.004 | 85.801 | 1.00 | 0.00 | xxxx | 55 |
| ATOM | 56 | CG2 | ILE | A | 9 | 90.886 | −4.563 | 87.149 | 1.00 | 0.00 | xxxx | 56 |
| ATOM | 57 | CD1 | ILE | A | 9 | 91.634 | −5.899 | 84.523 | 1.00 | 0.00 | xxxx | 57 |
| ATOM | 58 | N | TYR | A | 10 | 91.994 | −1.411 | 86.871 | 1.00 | 0.00 | xxxx | 58 |
| ATOM | 59 | CA | TYR | A | 10 | 92.119 | −0.166 | 87.630 | 1.00 | 0.00 | xxxx | 59 |
| ATOM | 60 | C | TYR | A | 10 | 91.894 | −0.426 | 89.119 | 1.00 | 0.00 | xxxx | 60 |
| ATOM | 61 | O | TYR | A | 10 | 92.540 | 0.190 | 89.952 | 1.00 | 0.00 | xxxx | 61 |
| ATOM | 62 | CB | TYR | A | 10 | 91.159 | 0.920 | 87.078 | 1.00 | 0.00 | xxxx | 62 |
| ATOM | 63 | CG | TYR | A | 10 | 89.811 | 1.015 | 87.771 | 1.00 | 0.00 | xxxx | 63 |
| ATOM | 64 | CD1 | TYR | A | 10 | 89.599 | 1.932 | 88.799 | 1.00 | 0.00 | xxxx | 64 |
| ATOM | 65 | CD2 | TYR | A | 10 | 88.745 | 0.201 | 87.388 | 1.00 | 0.00 | xxxx | 65 |
| ATOM | 66 | CE1 | TYR | A | 10 | 88.382 | 2.034 | 89.441 | 1.00 | 0.00 | xxxx | 66 |
| ATOM | 67 | CE2 | TYR | A | 10 | 87.514 | 0.286 | 88.034 | 1.00 | 0.00 | xxxx | 67 |
| ATOM | 68 | CZ | TYR | A | 10 | 87.340 | 1.210 | 89.055 | 1.00 | 0.00 | xxxx | 68 |
| ATOM | 69 | OH | TYR | A | 10 | 86.139 | 1.297 | 89.724 | 1.00 | 0.00 | xxxx | 69 |
| ATOM | 70 | N | LYS | A | 11 | 91.014 | −1.375 | 89.430 | 1.00 | 0.00 | xxxx | 70 |
| ATOM | 71 | CA | LYS | A | 11 | 90.672 | −1.735 | 90.799 | 1.00 | 0.00 | xxxx | 71 |
| ATOM | 72 | C | LYS | A | 11 | 90.059 | −3.128 | 90.779 | 1.00 | 0.00 | xxxx | 72 |
| ATOM | 73 | O | LYS | A | 11 | 88.993 | −3.327 | 90.195 | 1.00 | 0.00 | xxxx | 73 |
| ATOM | 74 | CB | LYS | A | 11 | 89.705 | −0.687 | 91.376 | 1.00 | 0.00 | xxxx | 74 |
| ATOM | 75 | CG | LYS | A | 11 | 88.958 | −1.080 | 92.621 | 1.00 | 0.00 | xxxx | 75 |
| ATOM | 76 | CD | LYS | A | 11 | 88.090 | 0.094 | 93.073 | 1.00 | 0.00 | xxxx | 76 |
| ATOM | 77 | CE | LYS | A | 11 | 87.341 | −0.224 | 94.359 | 1.00 | 0.00 | xxxx | 77 |
| ATOM | 78 | NZ | LYS | A | 11 | 88.210 | −0.910 | 95.353 | 1.00 | 0.00 | xxxx | 78 |
| ATOM | 79 | N | TYR | A | 12 | 90.728 | −4.107 | 91.389 | 1.00 | 0.00 | xxxx | 79 |
| ATOM | 80 | CA | TYR | A | 12 | 90.288 | −5.500 | 91.239 | 1.00 | 0.00 | xxxx | 80 |
| ATOM | 81 | C | TYR | A | 12 | 88.895 | −5.769 | 91.789 | 1.00 | 0.00 | xxxx | 81 |
| ATOM | 82 | O | TYR | A | 12 | 88.194 | −6.643 | 91.273 | 1.00 | 0.00 | xxxx | 82 |
| ATOM | 83 | CB | TYR | A | 12 | 91.252 | −6.472 | 91.934 | 1.00 | 0.00 | xxxx | 83 |
| ATOM | 84 | CG | TYR | A | 12 | 92.199 | −7.239 | 91.032 | 1.00 | 0.00 | xxxx | 84 |
| ATOM | 85 | CD1 | TYR | A | 12 | 92.817 | −6.627 | 89.945 | 1.00 | 0.00 | xxxx | 85 |
| ATOM | 86 | CD2 | TYR | A | 12 | 92.527 | −8.558 | 91.317 | 1.00 | 0.00 | xxxx | 86 |
| ATOM | 87 | CE1 | TYR | A | 12 | 93.724 | −7.315 | 89.146 | 1.00 | 0.00 | xxxx | 87 |
| ATOM | 88 | CE2 | TYR | A | 12 | 93.421 | −9.258 | 90.532 | 1.00 | 0.00 | xxxx | 88 |
| ATOM | 89 | CZ | TYR | A | 12 | 94.012 | −8.626 | 89.443 | 1.00 | 0.00 | xxxx | 89 |
| ATOM | 90 | OH | TYR | A | 12 | 94.917 | −9.315 | 88.675 | 1.00 | 0.00 | xxxx | 90 |
| ATOM | 91 | N | ASP | A | 13 | 88.491 | −5.051 | 92.831 | 1.00 | 0.00 | xxxx | 91 |
| ATOM | 92 | CA | ASP | A | 13 | 87.206 | −5.393 | 93.433 | 1.00 | 0.00 | xxxx | 92 |
| ATOM | 93 | C | ASP | A | 13 | 86.059 | −4.494 | 92.973 | 1.00 | 0.00 | xxxx | 93 |
| ATOM | 94 | O | ASP | A | 13 | 84.973 | −4.560 | 93.535 | 1.00 | 0.00 | xxxx | 94 |
| ATOM | 95 | CB | ASP | A | 13 | 87.311 | −5.409 | 94.960 | 1.00 | 0.00 | xxxx | 95 |
| ATOM | 96 | CG | ASP | A | 13 | 87.822 | −4.115 | 95.535 | 1.00 | 0.00 | xxxx | 96 |
| ATOM | 97 | OD1 | ASP | A | 13 | 88.328 | −3.272 | 94.778 | 1.00 | 0.00 | xxxx | 97 |
| ATOM | 98 | OD2 | ASP | A | 13 | 87.738 | −3.962 | 96.775 | 1.00 | 0.00 | xxxx | 98 |
| ATOM | 99 | N | ASP | A | 14 | 86.279 | −3.693 | 91.933 | 1.00 | 0.00 | xxxx | 99 |
| ATOM | 100 | CA | ASP | A | 14 | 85.161 | −3.118 | 91.169 | 1.00 | 0.00 | xxxx | 100 |
| ATOM | 101 | C | ASP | A | 14 | 84.313 | −4.302 | 90.691 | 1.00 | 0.00 | xxxx | 101 |
| ATOM | 102 | O | ASP | A | 14 | 84.858 | −5.221 | 90.091 | 1.00 | 0.00 | xxxx | 102 |
| ATOM | 103 | CB | ASP | A | 14 | 85.698 | −2.279 | 90.001 | 1.00 | 0.00 | xxxx | 103 |
| ATOM | 104 | CG | ASP | A | 14 | 84.601 | −1.711 | 89.124 | 1.00 | 0.00 | xxxx | 104 |
| ATOM | 105 | OD1 | ASP | A | 14 | 84.259 | −0.523 | 89.295 | 1.00 | 0.00 | xxxx | 105 |
| ATOM | 106 | OD2 | ASP | A | 14 | 84.090 | −2.449 | 88.260 | 1.00 | 0.00 | xxxx | 106 |
| ATOM | 107 | N | ASN | A | 15 | 83.007 | −4.316 | 90.980 | 1.00 | 0.00 | xxxx | 107 |
| ATOM | 108 | CA | ASN | A | 15 | 82.192 | −5.510 | 90.688 | 1.00 | 0.00 | xxxx | 108 |
| ATOM | 109 | C | ASN | A | 15 | 82.248 | −5.882 | 89.215 | 1.00 | 0.00 | xxxx | 109 |
| ATOM | 110 | O | ASN | A | 15 | 82.458 | −7.045 | 88.857 | 1.00 | 0.00 | xxxx | 110 |
| ATOM | 111 | CB | ASN | A | 15 | 80.724 | −5.312 | 91.091 | 1.00 | 0.00 | xxxx | 111 |
| ATOM | 112 | CG | ASN | A | 15 | 80.549 | −5.010 | 92.562 | 1.00 | 0.00 | xxxx | 112 |
| ATOM | 113 | OD1 | ASN | A | 15 | 81.029 | −3.996 | 93.054 | 1.00 | 0.00 | xxxx | 113 |
| ATOM | 114 | ND2 | ASN | A | 15 | 79.794 | −5.859 | 93.257 | 1.00 | 0.00 | xxxx | 114 |
| ATOM | 115 | N | PHE | A | 16 | 82.069 | −4.897 | 88.348 | 1.00 | 0.00 | xxxx | 115 |
| ATOM | 116 | CA | PHE | A | 16 | 82.097 | −5.199 | 86.927 | 1.00 | 0.00 | xxxx | 116 |
| ATOM | 117 | C | PHE | A | 16 | 83.483 | −5.634 | 86.450 | 1.00 | 0.00 | xxxx | 117 |
| ATOM | 118 | O | PHE | A | 16 | 83.621 | −6.611 | 85.693 | 1.00 | 0.00 | xxxx | 118 |
| ATOM | 119 | CB | PHE | A | 16 | 81.629 | −3.995 | 86.120 | 1.00 | 0.00 | xxxx | 119 |
| ATOM | 120 | CG | PHE | A | 16 | 81.657 | −4.235 | 84.646 | 1.00 | 0.00 | xxxx | 120 |
| ATOM | 121 | CD1 | PHE | A | 16 | 80.744 | −5.096 | 84.058 | 1.00 | 0.00 | xxxx | 121 |
| ATOM | 122 | CD2 | PHE | A | 16 | 82.608 | −3.621 | 83.856 | 1.00 | 0.00 | xxxx | 122 |
| ATOM | 123 | CE1 | PHE | A | 16 | 80.778 | −5.335 | 82.689 | 1.00 | 0.00 | xxxx | 123 |
| ATOM | 124 | CE2 | PHE | A | 16 | 82.649 | −3.859 | 82.482 | 1.00 | 0.00 | xxxx | 124 |
| ATOM | 125 | CZ | PHE | A | 16 | 81.731 | −4.720 | 81.909 | 1.00 | 0.00 | xxxx | 125 |
| ATOM | 126 | N | MET | A | 17 | 84.521 | −4.922 | 86.876 | 1.00 | 0.00 | xxxx | 126 |
| ATOM | 127 | CA | MET | A | 17 | 85.854 | −5.278 | 86.404 | 1.00 | 0.00 | xxxx | 127 |
| ATOM | 128 | C | MET | A | 17 | 86.328 | −6.622 | 86.968 | 1.00 | 0.00 | xxxx | 128 |
| ATOM | 129 | O | MET | A | 17 | 87.231 | −7.255 | 86.403 | 1.00 | 0.00 | xxxx | 129 |
| ATOM | 130 | CB | MET | A | 17 | 86.862 | −4.169 | 86.715 | 1.00 | 0.00 | xxxx | 130 |
| ATOM | 131 | CG | MET | A | 17 | 86.681 | −2.912 | 85.854 | 1.00 | 0.00 | xxxx | 131 |
| ATOM | 132 | SD | MET | A | 17 | 86.502 | −3.231 | 84.091 | 1.00 | 0.00 | xxxx | 132 |
| ATOM | 133 | CE | MET | A | 17 | 88.089 | −3.976 | 83.741 | 1.00 | 0.00 | xxxx | 133 |
| ATOM | 134 | N | SER | A | 18 | 85.738 | −7.047 | 88.082 | 1.00 | 0.00 | xxxx | 134 |
| ATOM | 135 | CA | SER | A | 18 | 85.999 | −8.383 | 88.605 | 1.00 | 0.00 | xxxx | 135 |
| ATOM | 136 | C | SER | A | 18 | 85.524 | −9.424 | 87.590 | 1.00 | 0.00 | xxxx | 136 |
| ATOM | 137 | O | SER | A | 18 | 86.219 | −10.411 | 87.320 | 1.00 | 0.00 | xxxx | 137 |
| ATOM | 138 | CB | SER | A | 18 | 85.309 | −8.577 | 89.957 | 1.00 | 0.00 | xxxx | 138 |
| ATOM | 139 | OG | SER | A | 18 | 85.592 | −9.854 | 90.498 | 1.00 | 0.00 | xxxx | 139 |
| ATOM | 140 | N | VAL | A | 19 | 84.352 | −9.186 | 87.004 | 1.00 | 0.00 | xxxx | 140 |
| ATOM | 141 | CA | VAL | A | 19 | 83.817 | −10.081 | 85.982 | 1.00 | 0.00 | xxxx | 141 |
| ATOM | 142 | C | VAL | A | 19 | 84.735 | −10.048 | 84.754 | 1.00 | 0.00 | xxxx | 142 |
| ATOM | 143 | O | VAL | A | 19 | 85.083 | −11.091 | 84.201 | 1.00 | 0.00 | xxxx | 143 |
| ATOM | 144 | CB | VAL | A | 19 | 82.362 | −9.716 | 85.597 | 1.00 | 0.00 | xxxx | 144 |
| ATOM | 145 | CG1 | VAL | A | 19 | 81.813 | −10.737 | 84.609 | 1.00 | 0.00 | xxxx | 145 |
| ATOM | 146 | CG2 | VAL | A | 19 | 81.465 | −9.656 | 86.832 | 1.00 | 0.00 | xxxx | 146 |
| ATOM | 147 | N | VAL | A | 20 | 85.130 | −8.851 | 84.331 | 1.00 | 0.00 | xxxx | 147 |
| ATOM | 148 | CA | VAL | A | 20 | 85.985 | −8.708 | 83.147 | 1.00 | 0.00 | xxxx | 148 |
| ATOM | 149 | C | VAL | A | 20 | 87.335 | −9.385 | 83.329 | 1.00 | 0.00 | xxxx | 149 |
| ATOM | 150 | O | VAL | A | 20 | 87.796 | −10.106 | 82.434 | 1.00 | 0.00 | xxxx | 150 |
| ATOM | 151 | CB | VAL | A | 20 | 86.174 | −7.219 | 82.781 | 1.00 | 0.00 | xxxx | 151 |
| ATOM | 152 | CG1 | VAL | A | 20 | 87.190 | −7.051 | 81.650 | 1.00 | 0.00 | xxxx | 152 |
| ATOM | 153 | CG2 | VAL | A | 20 | 84.842 | −6.607 | 82.385 | 1.00 | 0.00 | xxxx | 153 |
| ATOM | 154 | N | ARG | A | 21 | 87.983 | −9.163 | 84.468 | 1.00 | 0.00 | xxxx | 154 |
| ATOM | 155 | CA | ARG | A | 21 | 89.339 | −9.679 | 84.636 | 1.00 | 0.00 | xxxx | 155 |
| ATOM | 156 | C | ARG | A | 21 | 89.329 | −11.196 | 84.697 | 1.00 | 0.00 | xxxx | 156 |
| ATOM | 157 | O | ARG | A | 21 | 90.244 | −11.850 | 84.184 | 1.00 | 0.00 | xxxx | 157 |
| ATOM | 158 | CB | ARG | A | 21 | 90.007 | −9.074 | 85.875 | 1.00 | 0.00 | xxxx | 158 |
| ATOM | 159 | CG | ARG | A | 21 | 89.441 | −9.508 | 87.206 | 1.00 | 0.00 | xxxx | 159 |
| ATOM | 160 | CD | ARG | A | 21 | 90.137 | −8.696 | 88.303 | 1.00 | 0.00 | xxxx | 160 |
| ATOM | 161 | NE | ARG | A | 21 | 89.427 | −8.720 | 89.582 | 1.00 | 0.00 | xxxx | 161 |
| ATOM | 162 | CZ | ARG | A | 21 | 89.481 | −9.725 | 90.450 | 1.00 | 0.00 | xxxx | 162 |
| ATOM | 163 | NH1 | ARG | A | 21 | 90.195 | −10.809 | 90.165 | 1.00 | 0.00 | xxxx | 163 |
| ATOM | 164 | NH2 | ARG | A | 21 | 88.817 | −9.652 | 91.599 | 1.00 | 0.00 | xxxx | 164 |
| ATOM | 165 | N | LYS | A | 22 | 88.279 | −11.766 | 85.280 | 1.00 | 0.00 | xxxx | 165 |
| ATOM | 166 | CA | LYS | A | 22 | 88.183 | −13.214 | 85.344 | 1.00 | 0.00 | xxxx | 166 |
| ATOM | 167 | C | LYS | A | 22 | 87.954 | −13.790 | 83.955 | 1.00 | 0.00 | xxxx | 167 |
| ATOM | 168 | O | LYS | A | 22 | 88.479 | −14.857 | 83.616 | 1.00 | 0.00 | xxxx | 168 |
| ATOM | 169 | CB | LYS | A | 22 | 87.079 | −13.628 | 86.315 | 1.00 | 0.00 | xxxx | 169 |
| ATOM | 170 | CG | LYS | A | 22 | 87.475 | −13.286 | 87.751 | 1.00 | 0.00 | xxxx | 170 |
| ATOM | 171 | CD | LYS | A | 22 | 86.396 | −13.653 | 88.756 | 1.00 | 0.00 | xxxx | 171 |
| ATOM | 172 | CE | LYS | A | 22 | 86.756 | −13.110 | 90.132 | 1.00 | 0.00 | xxxx | 172 |
| ATOM | 173 | NZ | LYS | A | 22 | 85.780 | −13.518 | 91.180 | 1.00 | 0.00 | xxxx | 173 |
| ATOM | 174 | N | ALA | A | 23 | 87.209 | −13.060 | 83.132 | 1.00 | 0.00 | xxxx | 174 |
| ATOM | 175 | CA | ALA | A | 23 | 86.967 | −13.508 | 81.769 | 1.00 | 0.00 | xxxx | 175 |
| ATOM | 176 | C | ALA | A | 23 | 88.242 | −13.417 | 80.920 | 1.00 | 0.00 | xxxx | 176 |
| ATOM | 177 | O | ALA | A | 23 | 88.526 | −14.326 | 80.142 | 1.00 | 0.00 | xxxx | 177 |
| ATOM | 178 | CB | ALA | A | 23 | 85.836 | −12.705 | 81.144 | 1.00 | 0.00 | xxxx | 178 |
| ATOM | 179 | N | ILE | A | 24 | 89.023 | −12.349 | 81.084 | 1.00 | 0.00 | xxxx | 179 |
| ATOM | 180 | CA | ILE | A | 24 | 90.285 | −12.217 | 80.348 | 1.00 | 0.00 | xxxx | 180 |
| ATOM | 181 | C | ILE | A | 24 | 91.210 | −13.367 | 80.728 | 1.00 | 0.00 | xxxx | 181 |
| ATOM | 182 | O | ILE | A | 24 | 91.874 | −13.963 | 79.873 | 1.00 | 0.00 | xxxx | 182 |
| ATOM | 183 | CB | ILE | A | 24 | 90.973 | −10.856 | 80.630 | 1.00 | 0.00 | xxxx | 183 |
| ATOM | 184 | CG1 | ILE | A | 24 | 90.144 | −9.700 | 80.088 | 1.00 | 0.00 | xxxx | 184 |
| ATOM | 185 | CD1 | ILE | A | 24 | 90.752 | −8.339 | 80.389 | 1.00 | 0.00 | xxxx | 185 |
| ATOM | 186 | CG2 | ILE | A | 24 | 92.403 | −10.829 | 80.060 | 1.00 | 0.00 | xxxx | 186 |
| ATOM | 187 | N | GLU | A | 25 | 91.258 | −13.684 | 82.019 | 1.00 | 0.00 | xxxx | 187 |
| ATOM | 188 | CA | GLU | A | 25 | 92.092 | −14.790 | 82.474 | 1.00 | 0.00 | xxxx | 188 |
| ATOM | 189 | C | GLU | A | 25 | 91.698 | −16.104 | 81.797 | 1.00 | 0.00 | xxxx | 189 |
| ATOM | 190 | O | GLU | A | 25 | 92.564 | −16.883 | 81.400 | 1.00 | 0.00 | xxxx | 190 |
| ATOM | 191 | CB | GLU | A | 25 | 92.012 | −14.942 | 83.990 | 1.00 | 0.00 | xxxx | 191 |
| ATOM | 192 | CG | GLU | A | 25 | 93.068 | −15.883 | 84.537 | 1.00 | 0.00 | xxxx | 192 |
| ATOM | 193 | CD | GLU | A | 25 | 92.979 | −16.034 | 86.033 | 1.00 | 0.00 | xxxx | 193 |
| ATOM | 194 | OE1 | GLU | A | 25 | 92.080 | −16.763 | 86.503 | 1.00 | 0.00 | xxxx | 194 |
| ATOM | 195 | OE2 | GLU | A | 25 | 93.794 | −15.401 | 86.738 | 1.00 | 0.00 | xxxx | 195 |
| ATOM | 196 | N | GLN | A | 26 | 90.399 | −16.344 | 81.657 | 1.00 | 0.00 | xxxx | 196 |
| ATOM | 197 | CA | GLN | A | 26 | 89.935 | −17.567 | 81.007 | 1.00 | 0.00 | xxxx | 197 |
| ATOM | 198 | C | GLN | A | 26 | 90.310 | −17.606 | 79.525 | 1.00 | 0.00 | xxxx | 198 |
| ATOM | 199 | O | GLN | A | 26 | 90.686 | −18.654 | 79.004 | 1.00 | 0.00 | xxxx | 199 |
| ATOM | 200 | CB | GLN | A | 26 | 88.425 | −17.719 | 81.176 | 1.00 | 0.00 | xxxx | 200 |
| ATOM | 201 | CG | GLN | A | 26 | 88.009 | −17.965 | 82.613 | 1.00 | 0.00 | xxxx | 201 |
| ATOM | 202 | CD | GLN | A | 26 | 88.675 | −19.193 | 83.206 | 1.00 | 0.00 | xxxx | 202 |
| ATOM | 203 | OE1 | GLN | A | 26 | 89.258 | −19.136 | 84.288 | 1.00 | 0.00 | xxxx | 203 |
| ATOM | 204 | NE2 | GLN | A | 26 | 88.589 | −20.314 | 82.497 | 1.00 | 0.00 | xxxx | 204 |
| ATOM | 205 | N | ASP | A | 27 | 90.225 | −16.464 | 78.850 | 1.00 | 0.00 | xxxx | 205 |
| ATOM | 206 | CA | ASP | A | 27 | 90.642 | −16.386 | 77.454 | 1.00 | 0.00 | xxxx | 206 |
| ATOM | 207 | C | ASP | A | 27 | 92.129 | −16.674 | 77.293 | 1.00 | 0.00 | xxxx | 207 |
| ATOM | 208 | O | ASP | A | 27 | 92.544 | −17.394 | 76.367 | 1.00 | 0.00 | xxxx | 208 |
| ATOM | 209 | CB | ASP | A | 27 | 90.318 | −15.009 | 76.874 | 1.00 | 0.00 | xxxx | 209 |
| ATOM | 210 | CG | ASP | A | 27 | 88.819 | −14.753 | 76.777 | 1.00 | 0.00 | xxxx | 210 |
| ATOM | 211 | OD1 | ASP | A | 27 | 88.073 | −15.718 | 76.515 | 1.00 | 0.00 | xxxx | 211 |
| ATOM | 212 | OD2 | ASP | A | 27 | 88.386 | −13.591 | 76.968 | 1.00 | 0.00 | xxxx | 212 |
| ATOM | 213 | N | ALA | A | 28 | 92.941 | −16.146 | 78.202 | 1.00 | 0.00 | xxxx | 213 |
| ATOM | 214 | CA | ALA | A | 28 | 94.386 | −16.360 | 78.126 | 1.00 | 0.00 | xxxx | 214 |
| ATOM | 215 | C | ALA | A | 28 | 94.736 | −17.813 | 78.417 | 1.00 | 0.00 | xxxx | 215 |
| ATOM | 216 | O | ALA | A | 28 | 95.608 | −18.381 | 77.762 | 1.00 | 0.00 | xxxx | 216 |
| ATOM | 217 | CB | ALA | A | 28 | 95.123 | −15.427 | 79.088 | 1.00 | 0.00 | xxxx | 217 |
| ATOM | 218 | N | LYS | A | 29 | 94.047 | −18.412 | 79.388 | 1.00 | 0.00 | xxxx | 218 |
| ATOM | 219 | CA | LYS | A | 29 | 94.249 | −19.820 | 79.742 | 1.00 | 0.00 | xxxx | 219 |
| ATOM | 220 | C | LYS | A | 29 | 94.004 | −20.738 | 78.556 | 1.00 | 0.00 | xxxx | 220 |
| ATOM | 221 | O | LYS | A | 29 | 94.648 | −21.784 | 78.416 | 1.00 | 0.00 | xxxx | 221 |
| ATOM | 222 | CB | LYS | A | 29 | 93.326 | −20.223 | 80.895 | 1.00 | 0.00 | xxxx | 222 |
| ATOM | 223 | CG | LYS | A | 29 | 93.781 | −19.726 | 82.256 | 1.00 | 0.00 | xxxx | 223 |
| ATOM | 224 | CD | LYS | A | 29 | 92.810 | −20.153 | 83.347 | 1.00 | 0.00 | xxxx | 224 |
| ATOM | 225 | CE | LYS | A | 29 | 93.250 | −19.641 | 84.712 | 1.00 | 0.00 | xxxx | 225 |
| ATOM | 226 | NZ | LYS | A | 29 | 94.578 | −20.190 | 85.114 | 1.00 | 0.00 | xxxx | 226 |
| ATOM | 227 | N | ALA | A | 30 | 93.067 | −20.343 | 77.703 | 1.00 | 0.00 | xxxx | 227 |
| ATOM | 228 | CA | ALA | A | 30 | 92.696 | −21.144 | 76.544 | 1.00 | 0.00 | xxxx | 228 |
| ATOM | 229 | C | ALA | A | 30 | 93.770 | −21.106 | 75.461 | 1.00 | 0.00 | xxxx | 229 |
| ATOM | 230 | O | ALA | A | 30 | 93.772 | −21.936 | 74.549 | 1.00 | 0.00 | xxxx | 230 |
| ATOM | 231 | CB | ALA | A | 30 | 91.364 | −20.665 | 75.982 | 1.00 | 0.00 | xxxx | 231 |
| ATOM | 232 | N | ALA | A | 31 | 94.675 | −20.137 | 75.562 | 1.00 | 0.00 | xxxx | 232 |
| ATOM | 233 | CA | ALA | A | 31 | 95.722 | −19.923 | 74.566 | 1.00 | 0.00 | xxxx | 233 |
| ATOM | 234 | C | ALA | A | 31 | 97.061 | −20.427 | 75.093 | 1.00 | 0.00 | xxxx | 234 |
| ATOM | 235 | O | ALA | A | 31 | 97.695 | −19.760 | 75.905 | 1.00 | 0.00 | xxxx | 235 |
| ATOM | 236 | CB | ALA | A | 31 | 95.813 | −18.437 | 74.210 | 1.00 | 0.00 | xxxx | 236 |
| ATOM | 237 | N | PRO | A | 32 | 97.511 | −21.593 | 74.622 | 1.00 | 0.00 | xxxx | 237 |
| ATOM | 238 | CA | PRO | A | 32 | 98.679 | −22.204 | 75.270 | 1.00 | 0.00 | xxxx | 238 |
| ATOM | 239 | C | PRO | A | 32 | 99.986 | −21.419 | 75.097 | 1.00 | 0.00 | xxxx | 239 |
| ATOM | 240 | O | PRO | A | 32 | 100.924 | −21.659 | 75.859 | 1.00 | 0.00 | xxxx | 240 |
| ATOM | 241 | CB | PRO | A | 32 | 98.775 | −23.576 | 74.592 | 1.00 | 0.00 | xxxx | 241 |
| ATOM | 242 | CG | PRO | A | 32 | 98.064 | −23.417 | 73.288 | 1.00 | 0.00 | xxxx | 242 |
| ATOM | 243 | CD | PRO | A | 32 | 96.963 | −22.433 | 73.545 | 1.00 | 0.00 | xxxx | 243 |
| ATOM | 244 | N | ASP | A | 33 | 100.043 | −20.498 | 74.139 | 1.00 | 0.00 | xxxx | 244 |
| ATOM | 245 | CA | ASP | A | 33 | 101.282 | −19.774 | 73.856 | 1.00 | 0.00 | xxxx | 245 |
| ATOM | 246 | C | ASP | A | 33 | 101.348 | −18.412 | 74.543 | 1.00 | 0.00 | xxxx | 246 |
| ATOM | 247 | O | ASP | A | 33 | 102.256 | −17.629 | 74.271 | 1.00 | 0.00 | xxxx | 247 |
| ATOM | 248 | CB | ASP | A | 33 | 101.469 | −19.593 | 72.345 | 1.00 | 0.00 | xxxx | 248 |
| ATOM | 249 | CG | ASP | A | 33 | 100.328 | −18.833 | 71.692 | 1.00 | 0.00 | xxxx | 249 |
| ATOM | 250 | OD1 | ASP | A | 33 | 99.197 | −18.858 | 72.222 | 1.00 | 0.00 | xxxx | 250 |
| ATOM | 251 | OD2 | ASP | A | 33 | 100.564 | −18.214 | 70.629 | 1.00 | 0.00 | xxxx | 251 |
| ATOM | 252 | N | VAL | A | 34 | 100.404 | −18.145 | 75.442 | 1.00 | 0.00 | xxxx | 252 |
| ATOM | 253 | CA | VAL | A | 34 | 100.292 | −16.842 | 76.101 | 1.00 | 0.00 | xxxx | 253 |
| ATOM | 254 | C | VAL | A | 34 | 100.593 | −16.957 | 77.589 | 1.00 | 0.00 | xxxx | 254 |
| ATOM | 255 | O | VAL | A | 34 | 100.154 | −17.901 | 78.247 | 1.00 | 0.00 | xxxx | 255 |
| ATOM | 256 | CB | VAL | A | 34 | 98.890 | −16.238 | 75.893 | 1.00 | 0.00 | xxxx | 256 |
| ATOM | 257 | CG1 | VAL | A | 34 | 98.702 | −14.981 | 76.733 | 1.00 | 0.00 | xxxx | 257 |
| ATOM | 258 | CG2 | VAL | A | 34 | 98.670 | −15.936 | 74.435 | 1.00 | 0.00 | xxxx | 258 |
| ATOM | 259 | N | GLN | A | 35 | 101.361 | −16.004 | 78.108 | 1.00 | 0.00 | xxxx | 259 |
| ATOM | 260 | CA | GLN | A | 35 | 101.497 | −15.833 | 79.544 | 1.00 | 0.00 | xxxx | 260 |
| ATOM | 261 | C | GLN | A | 35 | 100.909 | −14.485 | 79.915 | 1.00 | 0.00 | xxxx | 261 |
| ATOM | 262 | O | GLN | A | 35 | 101.363 | −13.445 | 79.433 | 1.00 | 0.00 | xxxx | 262 |
| ATOM | 263 | CB | GLN | A | 35 | 102.946 | −15.910 | 79.997 | 1.00 | 0.00 | xxxx | 263 |
| ATOM | 264 | CG | GLN | A | 35 | 103.098 | −15.718 | 81.497 | 1.00 | 0.00 | xxxx | 264 |
| ATOM | 265 | CD | GLN | A | 35 | 104.544 | −15.694 | 81.942 | 1.00 | 0.00 | xxxx | 265 |
| ATOM | 266 | OE1 | GLN | A | 35 | 105.461 | −15.777 | 81.125 | 1.00 | 0.00 | xxxx | 266 |
| ATOM | 267 | NE2 | GLN | A | 35 | 104.757 | −15.577 | 83.249 | 1.00 | 0.00 | xxxx | 267 |
| ATOM | 268 | N | LEU | A | 36 | 99.886 | −14.513 | 80.755 | 1.00 | 0.00 | xxxx | 268 |
| ATOM | 269 | CA | LEU | A | 36 | 99.221 | −13.304 | 81.202 | 1.00 | 0.00 | xxxx | 269 |
| ATOM | 270 | C | LEU | A | 36 | 99.905 | −12.748 | 82.449 | 1.00 | 0.00 | xxxx | 270 |
| ATOM | 271 | O | LEU | A | 36 | 100.128 | −13.494 | 83.396 | 1.00 | 0.00 | xxxx | 271 |
| ATOM | 272 | CB | LEU | A | 36 | 97.760 | −13.610 | 81.499 | 1.00 | 0.00 | xxxx | 272 |
| ATOM | 273 | CG | LEU | A | 36 | 96.815 | −12.427 | 81.600 | 1.00 | 0.00 | xxxx | 273 |
| ATOM | 274 | CD1 | LEU | A | 36 | 96.635 | −11.829 | 80.218 | 1.00 | 0.00 | xxxx | 274 |
| ATOM | 275 | CD2 | LEU | A | 36 | 95.484 | −12.898 | 82.173 | 1.00 | 0.00 | xxxx | 275 |
| ATOM | 276 | N | LEU | A | 37 | 100.264 | −11.462 | 82.445 | 1.00 | 0.00 | xxxx | 276 |
| ATOM | 277 | CA | LEU | A | 37 | 100.825 | −10.832 | 83.641 | 1.00 | 0.00 | xxxx | 277 |
| ATOM | 278 | C | LEU | A | 37 | 99.845 | −9.749 | 84.060 | 1.00 | 0.00 | xxxx | 278 |
| ATOM | 279 | O | LEU | A | 37 | 99.835 | −8.664 | 83.473 | 1.00 | 0.00 | xxxx | 279 |
| ATOM | 280 | CB | LEU | A | 37 | 102.215 | −10.236 | 83.379 | 1.00 | 0.00 | xxxx | 280 |
| ATOM | 281 | CG | LEU | A | 37 | 103.399 | −11.161 | 83.055 | 1.00 | 0.00 | xxxx | 281 |
| ATOM | 282 | CD1 | LEU | A | 37 | 103.232 | −11.921 | 81.751 | 1.00 | 0.00 | xxxx | 282 |
| ATOM | 283 | CD2 | LEU | A | 37 | 104.663 | −10.338 | 82.976 | 1.00 | 0.00 | xxxx | 283 |
| ATOM | 284 | N | MET | A | 38 | 99.015 | −10.023 | 85.063 | 1.00 | 0.00 | xxxx | 284 |
| ATOM | 285 | CA | MET | A | 38 | 97.926 | −9.095 | 85.382 | 1.00 | 0.00 | xxxx | 285 |
| ATOM | 286 | C | MET | A | 38 | 98.051 | −8.491 | 86.779 | 1.00 | 0.00 | xxxx | 286 |
| ATOM | 287 | O | MET | A | 38 | 98.323 | −9.194 | 87.751 | 1.00 | 0.00 | xxxx | 287 |
| ATOM | 288 | CB | MET | A | 38 | 96.567 | −9.791 | 85.224 | 1.00 | 0.00 | xxxx | 288 |
| ATOM | 289 | CG | MET | A | 38 | 95.373 | −8.825 | 85.176 | 1.00 | 0.00 | xxxx | 289 |
| ATOM | 290 | SD | MET | A | 38 | 93.881 | −9.536 | 84.433 | 1.00 | 0.00 | xxxx | 290 |
| ATOM | 291 | CE | MET | A | 38 | 93.470 | −10.772 | 85.662 | 1.00 | 0.00 | xxxx | 291 |
| ATOM | 292 | N | ASN | A | 39 | 97.827 | −7.180 | 86.849 | 1.00 | 0.00 | xxxx | 292 |
| ATOM | 293 | CA | ASN | A | 39 | 98.037 | −6.372 | 88.051 | 1.00 | 0.00 | xxxx | 293 |
| ATOM | 294 | C | ASN | A | 39 | 96.781 | −5.654 | 88.498 | 1.00 | 0.00 | xxxx | 294 |
| ATOM | 295 | O | ASN | A | 39 | 95.965 | −5.230 | 87.672 | 1.00 | 0.00 | xxxx | 295 |
| ATOM | 296 | CB | ASN | A | 39 | 99.110 | −5.310 | 87.811 | 1.00 | 0.00 | xxxx | 296 |
| ATOM | 297 | CG | ASN | A | 39 | 100.506 | −5.893 | 87.698 | 1.00 | 0.00 | xxxx | 297 |
| ATOM | 298 | OD1 | ASN | A | 39 | 101.171 | −5.706 | 86.692 | 1.00 | 0.00 | xxxx | 298 |
| ATOM | 299 | ND2 | ASN | A | 39 | 100.961 | −6.585 | 88.746 | 1.00 | 0.00 | xxxx | 299 |
| ATOM | 300 | N | ASP | A | 40 | 96.664 | −5.511 | 89.812 | 1.00 | 0.00 | xxxx | 300 |
| ATOM | 301 | CA | ASP | A | 40 | 95.672 | −4.650 | 90.460 | 1.00 | 0.00 | xxxx | 301 |
| ATOM | 302 | C | ASP | A | 40 | 96.287 | −3.286 | 90.729 | 1.00 | 0.00 | xxxx | 302 |
| ATOM | 303 | O | ASP | A | 40 | 97.214 | −3.168 | 91.537 | 1.00 | 0.00 | xxxx | 303 |
| ATOM | 304 | CB | ASP | A | 40 | 95.198 | −5.300 | 91.771 | 1.00 | 0.00 | xxxx | 304 |
| ATOM | 305 | CG | ASP | A | 40 | 94.113 | −4.502 | 92.473 | 1.00 | 0.00 | xxxx | 305 |
| ATOM | 306 | OD1 | ASP | A | 40 | 93.553 | −3.564 | 91.862 | 1.00 | 0.00 | xxxx | 306 |
| ATOM | 307 | OD2 | ASP | A | 40 | 93.809 | −4.848 | 93.635 | 1.00 | 0.00 | xxxx | 307 |
| ATOM | 308 | N | SER | A | 41 | 95.772 | −2.247 | 90.076 | 1.00 | 0.00 | xxxx | 308 |
| ATOM | 309 | CA | SER | A | 41 | 96.320 | −0.910 | 90.271 | 1.00 | 0.00 | xxxx | 309 |
| ATOM | 310 | C | SER | A | 41 | 95.785 | −0.231 | 91.531 | 1.00 | 0.00 | xxxx | 310 |
| ATOM | 311 | O | SER | A | 41 | 96.189 | 0.875 | 91.841 | 1.00 | 0.00 | xxxx | 311 |
| ATOM | 312 | CB | SER | A | 41 | 96.033 | −0.043 | 89.042 | 1.00 | 0.00 | xxxx | 312 |
| ATOM | 313 | OG | SER | A | 41 | 96.652 | −0.624 | 87.893 | 1.00 | 0.00 | xxxx | 313 |
| ATOM | 314 | N | GLN | A | 42 | 94.886 | −0.899 | 92.258 | 1.00 | 0.00 | xxxx | 314 |
| ATOM | 315 | CA | GLN | A | 42 | 94.410 | −0.381 | 93.547 | 1.00 | 0.00 | xxxx | 315 |
| ATOM | 316 | C | GLN | A | 42 | 93.869 | 1.053 | 93.455 | 1.00 | 0.00 | xxxx | 316 |
| ATOM | 317 | O | GLN | A | 42 | 94.044 | 1.857 | 94.371 | 1.00 | 0.00 | xxxx | 317 |
| ATOM | 318 | CB | GLN | A | 42 | 95.537 | −0.455 | 94.588 | 1.00 | 0.00 | xxxx | 318 |
| ATOM | 319 | CG | GLN | A | 42 | 96.023 | −1.898 | 94.793 | 1.00 | 0.00 | xxxx | 319 |
| ATOM | 320 | CD | GLN | A | 42 | 96.973 | −2.066 | 95.967 | 1.00 | 0.00 | xxxx | 320 |
| ATOM | 321 | OE1 | GLN | A | 42 | 97.611 | −1.116 | 96.411 | 1.00 | 0.00 | xxxx | 321 |
| ATOM | 322 | NE2 | GLN | A | 42 | 97.071 | −3.296 | 96.474 | 1.00 | 0.00 | xxxx | 322 |
| ATOM | 323 | N | ASN | A | 43 | 93.207 | 1.350 | 92.336 | 1.00 | 0.00 | xxxx | 323 |
| ATOM | 324 | CA | ASN | A | 43 | 92.564 | 2.646 | 92.099 | 1.00 | 0.00 | xxxx | 324 |
| ATOM | 325 | C | ASN | A | 43 | 93.531 | 3.835 | 92.253 | 1.00 | 0.00 | xxxx | 325 |
| ATOM | 326 | O | ASN | A | 43 | 93.151 | 4.906 | 92.726 | 1.00 | 0.00 | xxxx | 326 |
| ATOM | 327 | CB | ASN | A | 43 | 91.361 | 2.817 | 93.037 | 1.00 | 0.00 | xxxx | 327 |
| ATOM | 328 | CG | ASN | A | 43 | 90.360 | 3.824 | 92.517 | 1.00 | 0.00 | xxxx | 328 |
| ATOM | 329 | OD1 | ASN | A | 43 | 90.301 | 4.088 | 91.319 | 1.00 | 0.00 | xxxx | 329 |
| ATOM | 330 | ND2 | ASN | A | 43 | 89.572 | 4.405 | 93.423 | 1.00 | 0.00 | xxxx | 330 |
| ATOM | 331 | N | ASP | A | 44 | 94.777 | 3.646 | 91.829 | 1.00 | 0.00 | xxxx | 331 |
| ATOM | 332 | CA | ASP | A | 44 | 95.807 | 4.677 | 91.943 | 1.00 | 0.00 | xxxx | 332 |
| ATOM | 333 | C | ASP | A | 44 | 96.592 | 4.708 | 90.630 | 1.00 | 0.00 | xxxx | 333 |
| ATOM | 334 | O | ASP | A | 44 | 97.292 | 3.749 | 90.300 | 1.00 | 0.00 | xxxx | 334 |
| ATOM | 335 | CB | ASP | A | 44 | 96.709 | 4.362 | 93.149 | 1.00 | 0.00 | xxxx | 335 |
| ATOM | 336 | CG | ASP | A | 44 | 97.743 | 5.434 | 93.437 | 1.00 | 0.00 | xxxx | 336 |
| ATOM | 337 | OD1 | ASP | A | 44 | 98.249 | 6.082 | 92.510 | 1.00 | 0.00 | xxxx | 337 |
| ATOM | 338 | OD2 | ASP | A | 44 | 98.075 | 5.609 | 94.631 | 1.00 | 0.00 | xxxx | 338 |
| ATOM | 339 | N | GLN | A | 45 | 96.493 | 5.798 | 89.872 | 1.00 | 0.00 | xxxx | 339 |
| ATOM | 340 | CA | GLN | A | 45 | 97.187 | 5.839 | 88.587 | 1.00 | 0.00 | xxxx | 340 |
| ATOM | 341 | C | GLN | A | 45 | 98.708 | 5.795 | 88.731 | 1.00 | 0.00 | xxxx | 341 |
| ATOM | 342 | O | GLN | A | 45 | 99.393 | 5.237 | 87.860 | 1.00 | 0.00 | xxxx | 342 |
| ATOM | 343 | CB | GLN | A | 45 | 96.780 | 7.085 | 87.788 | 1.00 | 0.00 | xxxx | 343 |
| ATOM | 344 | CG | GLN | A | 45 | 97.318 | 7.082 | 86.362 | 1.00 | 0.00 | xxxx | 344 |
| ATOM | 345 | CD | GLN | A | 45 | 96.752 | 5.928 | 85.546 | 1.00 | 0.00 | xxxx | 345 |
| ATOM | 346 | OE1 | GLN | A | 45 | 95.544 | 5.714 | 85.521 | 1.00 | 0.00 | xxxx | 346 |
| ATOM | 347 | NE2 | GLN | A | 45 | 97.630 | 5.174 | 84.884 | 1.00 | 0.00 | xxxx | 347 |
| ATOM | 348 | N | SER | A | 46 | 99.237 | 6.372 | 89.811 | 1.00 | 0.00 | xxxx | 348 |
| ATOM | 349 | CA | SER | A | 46 | 100.680 | 6.340 | 90.048 | 1.00 | 0.00 | xxxx | 349 |
| ATOM | 350 | C | SER | A | 46 | 101.150 | 4.901 | 90.204 | 1.00 | 0.00 | xxxx | 350 |
| ATOM | 351 | O | SER | A | 46 | 102.185 | 4.522 | 89.662 | 1.00 | 0.00 | xxxx | 351 |
| ATOM | 352 | CB | SER | A | 46 | 101.059 | 7.151 | 91.287 | 1.00 | 0.00 | xxxx | 352 |
| ATOM | 353 | OG | SER | A | 46 | 100.720 | 8.514 | 91.110 | 1.00 | 0.00 | xxxx | 353 |
| ATOM | 354 | N | LYS | A | 47 | 100.378 | 4.093 | 90.931 | 1.00 | 0.00 | xxxx | 354 |
| ATOM | 355 | CA | LYS | A | 47 | 100.684 | 2.667 | 91.039 | 1.00 | 0.00 | xxxx | 355 |
| ATOM | 356 | C | LYS | A | 47 | 100.614 | 1.975 | 89.685 | 1.00 | 0.00 | xxxx | 356 |
| ATOM | 357 | O | LYS | A | 47 | 101.463 | 1.146 | 89.362 | 1.00 | 0.00 | xxxx | 357 |
| ATOM | 358 | CB | LYS | A | 47 | 99.735 | 1.977 | 92.029 | 1.00 | 0.00 | xxxx | 358 |
| ATOM | 359 | CG | LYS | A | 47 | 99.961 | 2.404 | 93.475 | 1.00 | 0.00 | xxxx | 359 |
| ATOM | 360 | CD | LYS | A | 47 | 98.985 | 1.704 | 94.410 | 1.00 | 0.00 | xxxx | 360 |
| ATOM | 361 | CE | LYS | A | 47 | 99.264 | 2.081 | 95.854 | 1.00 | 0.00 | xxxx | 361 |
| ATOM | 362 | NZ | LYS | A | 47 | 98.268 | 1.501 | 96.800 | 1.00 | 0.00 | xxxx | 362 |
| ATOM | 363 | N | GLN | A | 48 | 99.592 | 2.305 | 88.898 | 1.00 | 0.00 | xxxx | 363 |
| ATOM | 364 | CA | GLN | A | 48 | 99.460 | 1.726 | 87.578 | 1.00 | 0.00 | xxxx | 364 |
| ATOM | 365 | C | GLN | A | 48 | 100.663 | 2.079 | 86.686 | 1.00 | 0.00 | xxxx | 365 |
| ATOM | 366 | O | GLN | A | 48 | 101.166 | 1.233 | 85.952 | 1.00 | 0.00 | xxxx | 366 |
| ATOM | 367 | CB | GLN | A | 48 | 98.160 | 2.189 | 86.915 | 1.00 | 0.00 | xxxx | 367 |
| ATOM | 368 | CG | GLN | A | 48 | 97.943 | 1.513 | 85.563 | 1.00 | 0.00 | xxxx | 368 |
| ATOM | 369 | CD | GLN | A | 48 | 96.543 | 1.713 | 85.022 | 1.00 | 0.00 | xxxx | 369 |
| ATOM | 370 | OE1 | GLN | A | 48 | 96.302 | 2.594 | 84.194 | 1.00 | 0.00 | xxxx | 370 |
| ATOM | 371 | NE2 | GLN | A | 48 | 95.611 | 0.888 | 85.477 | 1.00 | 0.00 | xxxx | 371 |
| ATOM | 372 | N | ASN | A | 49 | 101.128 | 3.320 | 86.760 | 1.00 | 0.00 | xxxx | 372 |
| ATOM | 373 | CA | ASN | A | 49 | 102.247 | 3.745 | 85.917 | 1.00 | 0.00 | xxxx | 373 |
| ATOM | 374 | C | ASN | A | 49 | 103.500 | 2.936 | 86.271 | 1.00 | 0.00 | xxxx | 374 |
| ATOM | 375 | O | ASN | A | 49 | 104.240 | 2.525 | 85.382 | 1.00 | 0.00 | xxxx | 375 |
| ATOM | 376 | CB | ASN | A | 49 | 102.506 | 5.251 | 86.061 | 1.00 | 0.00 | xxxx | 376 |
| ATOM | 377 | CG | ASN | A | 49 | 101.367 | 6.102 | 85.492 | 1.00 | 0.00 | xxxx | 377 |
| ATOM | 378 | OD1 | ASN | A | 49 | 100.536 | 5.616 | 84.729 | 1.00 | 0.00 | xxxx | 378 |
| ATOM | 379 | ND2 | ASN | A | 49 | 101.333 | 7.379 | 85.870 | 1.00 | 0.00 | xxxx | 379 |
| ATOM | 380 | N | ASP | A | 50 | 103.725 | 2.698 | 87.562 | 1.00 | 0.00 | xxxx | 380 |
| ATOM | 381 | CA | ASP | A | 50 | 104.856 | 1.874 | 87.982 | 1.00 | 0.00 | xxxx | 381 |
| ATOM | 382 | C | ASP | A | 50 | 104.697 | 0.440 | 87.487 | 1.00 | 0.00 | xxxx | 382 |
| ATOM | 383 | O | ASP | A | 50 | 105.672 | −0.216 | 87.089 | 1.00 | 0.00 | xxxx | 383 |
| ATOM | 384 | CB | ASP | A | 50 | 104.998 | 1.875 | 89.502 | 1.00 | 0.00 | xxxx | 384 |
| ATOM | 385 | CG | ASP | A | 50 | 105.413 | 3.222 | 90.053 | 1.00 | 0.00 | xxxx | 385 |
| ATOM | 386 | OD1 | ASP | A | 50 | 105.848 | 4.091 | 89.267 | 1.00 | 0.00 | xxxx | 386 |
| ATOM | 387 | OD2 | ASP | A | 50 | 105.311 | 3.403 | 91.286 | 1.00 | 0.00 | xxxx | 387 |
| ATOM | 388 | N | GLN | A | 51 | 103.464 | −0.055 | 87.526 | 1.00 | 0.00 | xxxx | 388 |
| ATOM | 389 | CA | GLN | A | 51 | 103.189 | −1.410 | 87.060 | 1.00 | 0.00 | xxxx | 389 |
| ATOM | 390 | C | GLN | A | 51 | 103.516 | −1.541 | 85.581 | 1.00 | 0.00 | xxxx | 390 |
| ATOM | 391 | O | GLN | A | 51 | 104.057 | −2.559 | 85.142 | 1.00 | 0.00 | xxxx | 391 |
| ATOM | 392 | CB | GLN | A | 51 | 101.725 | −1.778 | 87.334 | 1.00 | 0.00 | xxxx | 392 |
| ATOM | 393 | CG | GLN | A | 51 | 101.498 | −2.034 | 88.824 | 1.00 | 0.00 | xxxx | 393 |
| ATOM | 394 | CD | GLN | A | 51 | 100.046 | −2.147 | 89.231 | 1.00 | 0.00 | xxxx | 394 |
| ATOM | 395 | OE1 | GLN | A | 51 | 99.148 | −1.628 | 88.557 | 1.00 | 0.00 | xxxx | 395 |
| ATOM | 396 | NE2 | GLN | A | 51 | 99.806 | −2.809 | 90.361 | 1.00 | 0.00 | xxxx | 396 |
| ATOM | 397 | N | ILE | A | 52 | 103.182 | −0.515 | 84.811 | 1.00 | 0.00 | xxxx | 397 |
| ATOM | 398 | CA | ILE | A | 52 | 103.475 | −0.559 | 83.388 | 1.00 | 0.00 | xxxx | 398 |
| ATOM | 399 | C | ILE | A | 52 | 104.991 | −0.557 | 83.135 | 1.00 | 0.00 | xxxx | 399 |
| ATOM | 400 | O | ILE | A | 52 | 105.474 | −1.287 | 82.267 | 1.00 | 0.00 | xxxx | 400 |
| ATOM | 401 | CB | ILE | A | 52 | 102.741 | 0.587 | 82.668 | 1.00 | 0.00 | xxxx | 401 |
| ATOM | 402 | CG1 | ILE | A | 52 | 101.226 | 0.330 | 82.728 | 1.00 | 0.00 | xxxx | 402 |
| ATOM | 403 | CG2 | ILE | A | 52 | 103.220 | 0.723 | 81.220 | 1.00 | 0.00 | xxxx | 403 |
| ATOM | 404 | CD1 | ILE | A | 52 | 100.376 | 1.551 | 82.399 | 1.00 | 0.00 | xxxx | 404 |
| ATOM | 405 | N | ASP | A | 53 | 105.747 | 0.215 | 83.917 | 1.00 | 0.00 | xxxx | 405 |
| ATOM | 406 | CA | ASP | A | 53 | 107.214 | 0.162 | 83.824 | 1.00 | 0.00 | xxxx | 406 |
| ATOM | 407 | C | ASP | A | 53 | 107.728 | −1.266 | 84.031 | 1.00 | 0.00 | xxxx | 407 |
| ATOM | 408 | O | ASP | A | 53 | 108.629 | −1.727 | 83.310 | 1.00 | 0.00 | xxxx | 408 |
| ATOM | 409 | CB | ASP | A | 53 | 107.869 | 1.079 | 84.857 | 1.00 | 0.00 | xxxx | 409 |
| ATOM | 410 | CG | ASP | A | 53 | 107.717 | 2.547 | 84.531 | 1.00 | 0.00 | xxxx | 410 |
| ATOM | 411 | OD1 | ASP | A | 53 | 107.558 | 2.886 | 83.345 | 1.00 | 0.00 | xxxx | 411 |
| ATOM | 412 | OD2 | ASP | A | 53 | 107.786 | 3.365 | 85.474 | 1.00 | 0.00 | xxxx | 412 |
| ATOM | 413 | N | VAL | A | 54 | 107.188 | −1.952 | 85.036 | 1.00 | 0.00 | xxxx | 413 |
| ATOM | 414 | CA | VAL | A | 54 | 107.601 | −3.323 | 85.328 | 1.00 | 0.00 | xxxx | 414 |
| ATOM | 415 | C | VAL | A | 54 | 107.221 | −4.290 | 84.200 | 1.00 | 0.00 | xxxx | 415 |
| ATOM | 416 | O | VAL | A | 54 | 108.024 | −5.137 | 83.808 | 1.00 | 0.00 | xxxx | 416 |
| ATOM | 417 | CB | VAL | A | 54 | 107.011 | −3.791 | 86.677 | 1.00 | 0.00 | xxxx | 417 |
| ATOM | 418 | CG1 | VAL | A | 54 | 107.249 | −5.283 | 86.887 | 1.00 | 0.00 | xxxx | 418 |
| ATOM | 419 | CG2 | VAL | A | 54 | 107.617 | −2.982 | 87.823 | 1.00 | 0.00 | xxxx | 419 |
| ATOM | 420 | N | LEU | A | 55 | 106.011 | −4.156 | 83.664 | 1.00 | 0.00 | xxxx | 420 |
| ATOM | 421 | CA | LEU | A | 55 | 105.582 | −5.031 | 82.577 | 1.00 | 0.00 | xxxx | 421 |
| ATOM | 422 | C | LEU | A | 55 | 106.493 | −4.847 | 81.362 | 1.00 | 0.00 | xxxx | 422 |
| ATOM | 423 | O | LEU | A | 55 | 106.877 | −5.820 | 80.704 | 1.00 | 0.00 | xxxx | 423 |
| ATOM | 424 | CB | LEU | A | 55 | 104.120 | −4.747 | 82.216 | 1.00 | 0.00 | xxxx | 424 |
| ATOM | 425 | CG | LEU | A | 55 | 103.107 | −5.160 | 83.289 | 1.00 | 0.00 | xxxx | 425 |
| ATOM | 426 | CD1 | LEU | A | 55 | 101.717 | −4.586 | 83.008 | 1.00 | 0.00 | xxxx | 426 |
| ATOM | 427 | CD2 | LEU | A | 55 | 103.031 | −6.669 | 83.414 | 1.00 | 0.00 | xxxx | 427 |
| ATOM | 428 | N | LEU | A | 56 | 106.841 | −3.599 | 81.059 | 1.00 | 0.00 | xxxx | 428 |
| ATOM | 429 | CA | LEU | A | 56 | 107.753 | −3.336 | 79.949 | 1.00 | 0.00 | xxxx | 429 |
| ATOM | 430 | C | LEU | A | 56 | 109.150 | −3.903 | 80.226 | 1.00 | 0.00 | xxxx | 430 |
| ATOM | 431 | O | LEU | A | 56 | 109.793 | −4.445 | 79.325 | 1.00 | 0.00 | xxxx | 431 |
| ATOM | 432 | CB | LEU | A | 56 | 107.814 | −1.839 | 79.657 | 1.00 | 0.00 | xxxx | 432 |
| ATOM | 433 | CG | LEU | A | 56 | 106.512 | −1.310 | 79.039 | 1.00 | 0.00 | xxxx | 433 |
| ATOM | 434 | CD1 | LEU | A | 56 | 106.505 | 0.221 | 78.986 | 1.00 | 0.00 | xxxx | 434 |
| ATOM | 435 | CD2 | LEU | A | 56 | 106.269 | −1.906 | 77.650 | 1.00 | 0.00 | xxxx | 435 |
| ATOM | 436 | N | ALA | A | 57 | 109.615 | −3.812 | 81.472 | 1.00 | 0.00 | xxxx | 436 |
| ATOM | 437 | CA | ALA | A | 57 | 110.899 | −4.396 | 81.829 | 1.00 | 0.00 | xxxx | 437 |
| ATOM | 438 | C | ALA | A | 57 | 110.876 | −5.912 | 81.636 | 1.00 | 0.00 | xxxx | 438 |
| ATOM | 439 | O | ALA | A | 57 | 111.914 | −6.521 | 81.319 | 1.00 | 0.00 | xxxx | 439 |
| ATOM | 440 | CB | ALA | A | 57 | 111.272 | −4.039 | 83.279 | 1.00 | 0.00 | xxxx | 440 |
| ATOM | 441 | N | LYS | A | 58 | 109.699 | −6.511 | 81.821 | 1.00 | 0.00 | xxxx | 441 |
| ATOM | 442 | CA | LYS | A | 58 | 109.515 | −7.953 | 81.661 | 1.00 | 0.00 | xxxx | 442 |
| ATOM | 443 | C | LYS | A | 58 | 109.301 | −8.363 | 80.208 | 1.00 | 0.00 | xxxx | 443 |
| ATOM | 444 | O | LYS | A | 58 | 109.065 | −9.536 | 79.919 | 1.00 | 0.00 | xxxx | 444 |
| ATOM | 445 | CB | LYS | A | 58 | 108.349 | −8.439 | 82.523 | 1.00 | 0.00 | xxxx | 445 |
| ATOM | 446 | CG | LYS | A | 58 | 108.644 | −8.364 | 84.017 | 1.00 | 0.00 | xxxx | 446 |
| ATOM | 447 | CD | LYS | A | 58 | 107.485 | −8.852 | 84.854 | 1.00 | 0.00 | xxxx | 447 |
| ATOM | 448 | CE | LYS | A | 58 | 107.877 | −8.918 | 86.324 | 1.00 | 0.00 | xxxx | 448 |
| ATOM | 449 | NZ | LYS | A | 58 | 106.777 | −9.443 | 87.171 | 1.00 | 0.00 | xxxx | 449 |
| ATOM | 450 | N | GLY | A | 59 | 109.405 | −7.399 | 79.300 | 1.00 | 0.00 | xxxx | 450 |
| ATOM | 451 | CA | GLY | A | 59 | 109.364 | −7.687 | 77.882 | 1.00 | 0.00 | xxxx | 451 |
| ATOM | 452 | C | GLY | A | 59 | 108.011 | −8.128 | 77.353 | 1.00 | 0.00 | xxxx | 452 |
| ATOM | 453 | O | GLY | A | 59 | 107.951 | −8.956 | 76.442 | 1.00 | 0.00 | xxxx | 453 |
| ATOM | 454 | N | VAL | A | 60 | 106.925 | −7.596 | 77.908 | 1.00 | 0.00 | xxxx | 454 |
| ATOM | 455 | CA | VAL | A | 60 | 105.607 | −7.915 | 77.365 | 1.00 | 0.00 | xxxx | 455 |
| ATOM | 456 | C | VAL | A | 60 | 105.555 | −7.444 | 75.916 | 1.00 | 0.00 | xxxx | 456 |
| ATOM | 457 | O | VAL | A | 60 | 106.153 | −6.430 | 75.552 | 1.00 | 0.00 | xxxx | 457 |
| ATOM | 458 | CB | VAL | A | 60 | 104.460 | −7.298 | 78.184 | 1.00 | 0.00 | xxxx | 458 |
| ATOM | 459 | CG1 | VAL | A | 60 | 104.425 | −7.880 | 79.587 | 1.00 | 0.00 | xxxx | 459 |
| ATOM | 460 | CG2 | VAL | A | 60 | 104.512 | −5.757 | 78.190 | 1.00 | 0.00 | xxxx | 460 |
| ATOM | 461 | N | LYS | A | 61 | 104.831 | −8.195 | 75.093 | 1.00 | 0.00 | xxxx | 461 |
| ATOM | 462 | CA | LYS | A | 61 | 104.708 | −7.860 | 73.679 | 1.00 | 0.00 | xxxx | 462 |
| ATOM | 463 | C | LYS | A | 61 | 103.444 | −7.049 | 73.388 | 1.00 | 0.00 | xxxx | 463 |
| ATOM | 464 | O | LYS | A | 61 | 103.270 | −6.551 | 72.280 | 1.00 | 0.00 | xxxx | 464 |
| ATOM | 465 | CB | LYS | A | 61 | 104.736 | −9.135 | 72.830 | 1.00 | 0.00 | xxxx | 465 |
| ATOM | 466 | CG | LYS | A | 61 | 106.049 | −9.896 | 72.930 | 1.00 | 0.00 | xxxx | 466 |
| ATOM | 467 | CD | LYS | A | 61 | 106.112 | −11.014 | 71.902 | 1.00 | 0.00 | xxxx | 467 |
| ATOM | 468 | CE | LYS | A | 61 | 106.273 | −10.456 | 70.491 | 1.00 | 0.00 | xxxx | 468 |
| ATOM | 469 | NZ | LYS | A | 61 | 106.157 | −11.505 | 69.426 | 1.00 | 0.00 | xxxx | 469 |
| ATOM | 470 | N | ALA | A | 62 | 102.561 | −6.938 | 74.374 | 1.00 | 0.00 | xxxx | 470 |
| ATOM | 471 | CA | ALA | A | 62 | 101.392 | −6.064 | 74.278 | 1.00 | 0.00 | xxxx | 471 |
| ATOM | 472 | C | ALA | A | 62 | 100.930 | −5.706 | 75.670 | 1.00 | 0.00 | xxxx | 472 |
| ATOM | 473 | O | ALA | A | 62 | 101.185 | −6.447 | 76.623 | 1.00 | 0.00 | xxxx | 473 |
| ATOM | 474 | CB | ALA | A | 62 | 100.254 | −6.732 | 73.503 | 1.00 | 0.00 | xxxx | 474 |
| ATOM | 475 | N | LEU | A | 63 | 100.280 | −4.553 | 75.787 | 1.00 | 0.00 | xxxx | 475 |
| ATOM | 476 | CA | LEU | A | 63 | 99.649 | −4.122 | 77.034 | 1.00 | 0.00 | xxxx | 476 |
| ATOM | 477 | C | LEU | A | 63 | 98.146 | −3.994 | 76.833 | 1.00 | 0.00 | xxxx | 477 |
| ATOM | 478 | O | LEU | A | 63 | 97.712 | −3.472 | 75.820 | 1.00 | 0.00 | xxxx | 478 |
| ATOM | 479 | CB | LEU | A | 63 | 100.211 | −2.774 | 77.479 | 1.00 | 0.00 | xxxx | 479 |
| ATOM | 480 | CG | LEU | A | 63 | 101.669 | −2.776 | 77.919 | 1.00 | 0.00 | xxxx | 480 |
| ATOM | 481 | CD1 | LEU | A | 63 | 102.226 | −1.365 | 77.884 | 1.00 | 0.00 | xxxx | 481 |
| ATOM | 482 | CD2 | LEU | A | 63 | 101.797 | −3.368 | 79.321 | 1.00 | 0.00 | xxxx | 482 |
| ATOM | 483 | N | ALA | A | 64 | 97.360 | −4.466 | 77.798 | 1.00 | 0.00 | xxxx | 483 |
| ATOM | 484 | CA | ALA | A | 64 | 95.934 | −4.163 | 77.857 | 1.00 | 0.00 | xxxx | 484 |
| ATOM | 485 | C | ALA | A | 64 | 95.720 | −3.374 | 79.139 | 1.00 | 0.00 | xxxx | 485 |
| ATOM | 486 | O | ALA | A | 64 | 95.967 | −3.887 | 80.233 | 1.00 | 0.00 | xxxx | 486 |
| ATOM | 487 | CB | ALA | A | 64 | 95.091 | −5.442 | 77.841 | 1.00 | 0.00 | xxxx | 487 |
| ATOM | 488 | N | ILE | A | 65 | 95.281 | −2.125 | 79.008 | 1.00 | 0.00 | xxxx | 488 |
| ATOM | 489 | CA | ILE | A | 65 | 95.305 | −1.184 | 80.125 | 1.00 | 0.00 | xxxx | 489 |
| ATOM | 490 | C | ILE | A | 65 | 93.900 | −0.669 | 80.430 | 1.00 | 0.00 | xxxx | 490 |
| ATOM | 491 | O | ILE | A | 65 | 93.240 | −0.078 | 79.579 | 1.00 | 0.00 | xxxx | 491 |
| ATOM | 492 | CB | ILE | A | 65 | 96.250 | 0.003 | 79.819 | 1.00 | 0.00 | xxxx | 492 |
| ATOM | 493 | CG1 | ILE | A | 65 | 97.671 | −0.497 | 79.500 | 1.00 | 0.00 | xxxx | 493 |
| ATOM | 494 | CG2 | ILE | A | 65 | 96.178 | 1.044 | 80.964 | 1.00 | 0.00 | xxxx | 494 |
| ATOM | 495 | CD1 | ILE | A | 65 | 98.341 | −1.250 | 80.648 | 1.00 | 0.00 | xxxx | 495 |
| ATOM | 496 | N | ASN | A | 66 | 93.433 | −0.941 | 81.640 | 1.00 | 0.00 | xxxx | 496 |
| ATOM | 497 | CA | ASN | A | 66 | 92.196 | −0.377 | 82.162 | 1.00 | 0.00 | xxxx | 497 |
| ATOM | 498 | C | ASN | A | 66 | 92.595 | 0.789 | 83.070 | 1.00 | 0.00 | xxxx | 498 |
| ATOM | 499 | O | ASN | A | 66 | 92.954 | 0.574 | 84.227 | 1.00 | 0.00 | xxxx | 499 |
| ATOM | 500 | CB | ASN | A | 66 | 91.427 | −1.500 | 82.892 | 1.00 | 0.00 | xxxx | 500 |
| ATOM | 501 | CG | ASN | A | 66 | 90.188 | −1.022 | 83.635 | 1.00 | 0.00 | xxxx | 501 |
| ATOM | 502 | OD1 | ASN | A | 66 | 89.988 | −1.389 | 84.797 | 1.00 | 0.00 | xxxx | 502 |
| ATOM | 503 | ND2 | ASN | A | 66 | 89.317 | −0.271 | 82.953 | 1.00 | 0.00 | xxxx | 503 |
| ATOM | 504 | N | LEU | A | 67 | 92.595 | 2.007 | 82.519 | 1.00 | 0.00 | xxxx | 504 |
| ATOM | 505 | CA | LEU | A | 67 | 93.111 | 3.187 | 83.213 | 1.00 | 0.00 | xxxx | 505 |
| ATOM | 506 | C | LEU | A | 67 | 92.402 | 3.474 | 84.526 | 1.00 | 0.00 | xxxx | 506 |
| ATOM | 507 | O | LEU | A | 67 | 91.196 | 3.295 | 84.638 | 1.00 | 0.00 | xxxx | 507 |
| ATOM | 508 | CB | LEU | A | 67 | 92.999 | 4.416 | 82.306 | 1.00 | 0.00 | xxxx | 508 |
| ATOM | 509 | CG | LEU | A | 67 | 93.959 | 4.418 | 81.114 | 1.00 | 0.00 | xxxx | 509 |
| ATOM | 510 | CD1 | LEU | A | 67 | 93.473 | 5.426 | 80.071 | 1.00 | 0.00 | xxxx | 510 |
| ATOM | 511 | CD2 | LEU | A | 67 | 95.356 | 4.773 | 81.575 | 1.00 | 0.00 | xxxx | 511 |
| ATOM | 512 | N | VAL | A | 68 | 93.164 | 3.908 | 85.529 | 1.00 | 0.00 | xxxx | 512 |
| ATOM | 513 | CA | VAL | A | 68 | 92.548 | 4.499 | 86.712 | 1.00 | 0.00 | xxxx | 513 |
| ATOM | 514 | C | VAL | A | 68 | 92.007 | 5.883 | 86.328 | 1.00 | 0.00 | xxxx | 514 |
| ATOM | 515 | O | VAL | A | 68 | 90.839 | 6.205 | 86.572 | 1.00 | 0.00 | xxxx | 515 |
| ATOM | 516 | CB | VAL | A | 68 | 93.543 | 4.601 | 87.879 | 1.00 | 0.00 | xxxx | 516 |
| ATOM | 517 | CG1 | VAL | A | 68 | 92.888 | 5.299 | 89.074 | 1.00 | 0.00 | xxxx | 517 |
| ATOM | 518 | CG2 | VAL | A | 68 | 94.045 | 3.214 | 88.276 | 1.00 | 0.00 | xxxx | 518 |
| ATOM | 519 | N | ASP | A | 69 | 92.881 | 6.688 | 85.728 | 1.00 | 0.00 | xxxx | 519 |
| ATOM | 520 | CA | ASP | A | 69 | 92.566 | 8.048 | 85.273 | 1.00 | 0.00 | xxxx | 520 |
| ATOM | 521 | C | ASP | A | 69 | 92.653 | 8.091 | 83.749 | 1.00 | 0.00 | xxxx | 521 |
| ATOM | 522 | O | ASP | A | 69 | 93.722 | 7.908 | 83.182 | 1.00 | 0.00 | xxxx | 522 |
| ATOM | 523 | CB | ASP | A | 69 | 93.534 | 9.049 | 85.904 | 1.00 | 0.00 | xxxx | 523 |
| ATOM | 524 | CG | ASP | A | 69 | 93.300 | 10.481 | 85.454 | 1.00 | 0.00 | xxxx | 524 |
| ATOM | 525 | OD1 | ASP | A | 69 | 92.338 | 10.757 | 84.700 | 1.00 | 0.00 | xxxx | 525 |
| ATOM | 526 | OD2 | ASP | A | 69 | 94.096 | 11.344 | 85.886 | 1.00 | 0.00 | xxxx | 526 |
| ATOM | 527 | N | PRO | A | 70 | 91.519 | 8.324 | 83.081 | 1.00 | 0.00 | xxxx | 527 |
| ATOM | 528 | CA | PRO | A | 70 | 91.546 | 8.357 | 81.613 | 1.00 | 0.00 | xxxx | 528 |
| ATOM | 529 | C | PRO | A | 70 | 92.534 | 9.376 | 81.041 | 1.00 | 0.00 | xxxx | 529 |
| ATOM | 530 | O | PRO | A | 70 | 93.051 | 9.184 | 79.938 | 1.00 | 0.00 | xxxx | 530 |
| ATOM | 531 | CB | PRO | A | 70 | 90.108 | 8.731 | 81.247 | 1.00 | 0.00 | xxxx | 531 |
| ATOM | 532 | CG | PRO | A | 70 | 89.289 | 8.251 | 82.394 | 1.00 | 0.00 | xxxx | 532 |
| ATOM | 533 | CD | PRO | A | 70 | 90.154 | 8.424 | 83.624 | 1.00 | 0.00 | xxxx | 533 |
| ATOM | 534 | N | ALA | A | 71 | 92.813 | 10.440 | 81.784 | 1.00 | 0.00 | xxxx | 534 |
| ATOM | 535 | CA | ALA | A | 71 | 93.717 | 11.475 | 81.292 | 1.00 | 0.00 | xxxx | 535 |
| ATOM | 536 | C | ALA | A | 71 | 95.163 | 10.987 | 81.229 | 1.00 | 0.00 | xxxx | 536 |
| ATOM | 537 | O | ALA | A | 71 | 96.015 | 11.647 | 80.638 | 1.00 | 0.00 | xxxx | 537 |
| ATOM | 538 | CB | ALA | A | 71 | 93.618 | 12.724 | 82.166 | 1.00 | 0.00 | xxxx | 538 |
| ATOM | 539 | N | ALA | A | 72 | 95.439 | 9.831 | 81.834 | 1.00 | 0.00 | xxxx | 539 |
| ATOM | 540 | CA | ALA | A | 72 | 96.795 | 9.299 | 81.874 | 1.00 | 0.00 | xxxx | 540 |
| ATOM | 541 | C | ALA | A | 72 | 97.136 | 8.467 | 80.655 | 1.00 | 0.00 | xxxx | 541 |
| ATOM | 542 | O | ALA | A | 72 | 98.216 | 7.874 | 80.598 | 1.00 | 0.00 | xxxx | 542 |
| ATOM | 543 | CB | ALA | A | 72 | 96.998 | 8.462 | 83.127 | 1.00 | 0.00 | xxxx | 543 |
| ATOM | 544 | N | ALA | A | 73 | 96.221 | 8.396 | 79.693 | 1.00 | 0.00 | xxxx | 544 |
| ATOM | 545 | CA | ALA | A | 73 | 96.492 | 7.631 | 78.472 | 1.00 | 0.00 | xxxx | 545 |
| ATOM | 546 | C | ALA | A | 73 | 97.802 | 8.065 | 77.811 | 1.00 | 0.00 | xxxx | 546 |
| ATOM | 547 | O | ALA | A | 73 | 98.565 | 7.229 | 77.328 | 1.00 | 0.00 | xxxx | 547 |
| ATOM | 548 | CB | ALA | A | 73 | 95.333 | 7.765 | 77.492 | 1.00 | 0.00 | xxxx | 548 |
| ATOM | 549 | N | GLY | A | 74 | 98.058 | 9.373 | 77.788 | 1.00 | 0.00 | xxxx | 549 |
| ATOM | 550 | CA | GLY | A | 74 | 99.286 | 9.886 | 77.203 | 1.00 | 0.00 | xxxx | 550 |
| ATOM | 551 | C | GLY | A | 74 | 100.541 | 9.357 | 77.875 | 1.00 | 0.00 | xxxx | 551 |
| ATOM | 552 | O | GLY | A | 74 | 101.514 | 8.988 | 77.214 | 1.00 | 0.00 | xxxx | 552 |
| ATOM | 553 | N | THR | A | 75 | 100.518 | 9.335 | 79.204 | 1.00 | 0.00 | xxxx | 553 |
| ATOM | 554 | CA | THR | A | 75 | 101.631 | 8.825 | 79.990 | 1.00 | 0.00 | xxxx | 554 |
| ATOM | 555 | C | THR | A | 75 | 101.904 | 7.368 | 79.631 | 1.00 | 0.00 | xxxx | 555 |
| ATOM | 556 | O | THR | A | 75 | 103.051 | 6.971 | 79.433 | 1.00 | 0.00 | xxxx | 556 |
| ATOM | 557 | CB | THR | A | 75 | 101.340 | 8.945 | 81.489 | 1.00 | 0.00 | xxxx | 557 |
| ATOM | 558 | OG1 | THR | A | 75 | 101.223 | 10.330 | 81.831 | 1.00 | 0.00 | xxxx | 558 |
| ATOM | 559 | CG2 | THR | A | 75 | 102.458 | 8.299 | 82.317 | 1.00 | 0.00 | xxxx | 559 |
| ATOM | 560 | N | VAL | A | 76 | 100.838 | 6.576 | 79.550 | 1.00 | 0.00 | xxxx | 560 |
| ATOM | 561 | CA | VAL | A | 76 | 100.961 | 5.165 | 79.228 | 1.00 | 0.00 | xxxx | 561 |
| ATOM | 562 | C | VAL | A | 76 | 101.516 | 4.973 | 77.818 | 1.00 | 0.00 | xxxx | 562 |
| ATOM | 563 | O | VAL | A | 76 | 102.418 | 4.160 | 77.598 | 1.00 | 0.00 | xxxx | 563 |
| ATOM | 564 | CB | VAL | A | 76 | 99.600 | 4.457 | 79.384 | 1.00 | 0.00 | xxxx | 564 |
| ATOM | 565 | CG1 | VAL | A | 76 | 99.679 | 3.013 | 78.879 | 1.00 | 0.00 | xxxx | 565 |
| ATOM | 566 | CG2 | VAL | A | 76 | 99.164 | 4.498 | 80.850 | 1.00 | 0.00 | xxxx | 566 |
| ATOM | 567 | N | ILE | A | 77 | 100.990 | 5.747 | 76.874 | 1.00 | 0.00 | xxxx | 567 |
| ATOM | 568 | CA | ILE | A | 77 | 101.426 | 5.663 | 75.477 | 1.00 | 0.00 | xxxx | 568 |
| ATOM | 569 | C | ILE | A | 77 | 102.916 | 5.991 | 75.341 | 1.00 | 0.00 | xxxx | 569 |
| ATOM | 570 | O | ILE | A | 77 | 103.650 | 5.327 | 74.601 | 1.00 | 0.00 | xxxx | 570 |
| ATOM | 571 | CB | ILE | A | 77 | 100.580 | 6.595 | 74.594 | 1.00 | 0.00 | xxxx | 571 |
| ATOM | 572 | CG1 | ILE | A | 77 | 99.207 | 5.972 | 74.345 | 1.00 | 0.00 | xxxx | 572 |
| ATOM | 573 | CG2 | ILE | A | 77 | 101.303 | 6.905 | 73.287 | 1.00 | 0.00 | xxxx | 573 |
| ATOM | 574 | CD1 | ILE | A | 77 | 98.218 | 6.927 | 73.704 | 1.00 | 0.00 | xxxx | 574 |
| ATOM | 575 | N | GLU | A | 78 | 103.371 | 7.007 | 76.065 | 1.00 | 0.00 | xxxx | 575 |
| ATOM | 576 | C | GLU | A | 78 | 105.680 | 6.225 | 76.455 | 1.00 | 0.00 | xxxx | 576 |
| ATOM | 577 | 0 | GLU | A | 78 | 106.718 | 5.967 | 75.839 | 1.00 | 0.00 | xxxx | 577 |
| ATOM | 578 | CA | GLU | A | 78 | 104.781 | 7.389 | 76.018 | 1.00 | 0.00 | xxxx | 578 |
| ATOM | 579 | CB | GLU | A | 78 | 105.029 | 8.620 | 76.899 | 1.00 | 0.00 | xxxx | 579 |
| ATOM | 580 | CG | GLU | A | 78 | 106.383 | 9.281 | 76.690 | 1.00 | 0.00 | xxxx | 580 |
| ATOM | 581 | CD | GLU | A | 78 | 107.463 | 8.732 | 77.605 | 1.00 | 0.00 | xxxx | 581 |
| ATOM | 582 | OE1 | GLU | A | 78 | 107.122 | 8.181 | 78.674 | 1.00 | 0.00 | xxxx | 582 |
| ATOM | 583 | OE2 | GLU | A | 78 | 108.658 | 8.853 | 77.255 | 1.00 | 0.00 | xxxx | 583 |
| ATOM | 584 | N | LYS | A | 79 | 105.275 | 5.512 | 77.504 | 1.00 | 0.00 | xxxx | 584 |
| ATOM | 585 | CA | LYS | A | 79 | 106.071 | 4.387 | 77.979 | 1.00 | 0.00 | xxxx | 585 |
| ATOM | 586 | C | LYS | A | 79 | 106.086 | 3.283 | 76.931 | 1.00 | 0.00 | xxxx | 586 |
| ATOM | 587 | O | LYS | A | 79 | 107.145 | 2.742 | 76.596 | 1.00 | 0.00 | xxxx | 587 |
| ATOM | 588 | CB | LYS | A | 79 | 105.533 | 3.860 | 79.310 | 1.00 | 0.00 | xxxx | 588 |
| ATOM | 589 | CG | LYS | A | 79 | 105.667 | 4.829 | 80.472 | 1.00 | 0.00 | xxxx | 589 |
| ATOM | 590 | CD | LYS | A | 79 | 104.967 | 4.284 | 81.707 | 1.00 | 0.00 | xxxx | 590 |
| ATOM | 591 | CE | LYS | A | 79 | 105.041 | 5.261 | 82.886 | 1.00 | 0.00 | xxxx | 591 |
| ATOM | 592 | NZ | LYS | A | 79 | 106.441 | 5.513 | 83.323 | 1.00 | 0.00 | xxxx | 592 |
| ATOM | 593 | N | ALA | A | 80 | 104.912 | 2.963 | 76.396 | 1.00 | 0.00 | xxxx | 593 |
| ATOM | 594 | CA | ALA | A | 80 | 104.798 | 1.881 | 75.427 | 1.00 | 0.00 | xxxx | 594 |
| ATOM | 595 | C | ALA | A | 80 | 105.539 | 2.204 | 74.126 | 1.00 | 0.00 | xxxx | 595 |
| ATOM | 596 | O | ALA | A | 80 | 106.202 | 1.333 | 73.546 | 1.00 | 0.00 | xxxx | 596 |
| ATOM | 597 | CB | ALA | A | 80 | 103.346 | 1.591 | 75.143 | 1.00 | 0.00 | xxxx | 597 |
| ATOM | 598 | O | ARG | A | 81 | 108.081 | 3.246 | 71.373 | 1.00 | 0.00 | xxxx | 598 |
| ATOM | 599 | N | ARG | A | 81 | 105.423 | 3.453 | 73.679 | 1.00 | 0.00 | xxxx | 599 |
| ATOM | 600 | CA | ARG | A | 81 | 106.016 | 3.886 | 72.408 | 1.00 | 0.00 | xxxx | 600 |
| ATOM | 601 | C | ARG | A | 81 | 107.524 | 3.653 | 72.391 | 1.00 | 0.00 | xxxx | 601 |
| ATOM | 602 | CB | ARG | A | 81 | 105.708 | 5.365 | 72.151 | 1.00 | 0.00 | xxxx | 602 |
| ATOM | 603 | CG | ARG | A | 81 | 106.197 | 5.872 | 70.789 | 1.00 | 0.00 | xxxx | 603 |
| ATOM | 604 | CD | ARG | A | 81 | 106.272 | 7.394 | 70.728 | 1.00 | 0.00 | xxxx | 604 |
| ATOM | 605 | NE | ARG | A | 81 | 105.027 | 8.050 | 71.122 | 1.00 | 0.00 | xxxx | 605 |
| ATOM | 606 | CZ | ARG | A | 81 | 104.003 | 8.258 | 70.302 | 1.00 | 0.00 | xxxx | 606 |
| ATOM | 607 | NH1 | ARG | A | 81 | 102.913 | 8.870 | 70.752 | 1.00 | 0.00 | xxxx | 607 |
| ATOM | 608 | NH2 | ARG | A | 81 | 104.058 | 7.843 | 69.041 | 1.00 | 0.00 | xxxx | 608 |
| ATOM | 609 | N | GLY | A | 82 | 108.172 | 3.884 | 73.530 | 1.00 | 0.00 | xxxx | 609 |
| ATOM | 610 | CA | GLY | A | 82 | 109.614 | 3.736 | 73.627 | 1.00 | 0.00 | xxxx | 610 |
| ATOM | 611 | C | GLY | A | 82 | 110.090 | 2.319 | 73.370 | 1.00 | 0.00 | xxxx | 611 |
| ATOM | 612 | O | GLY | A | 82 | 111.231 | 2.085 | 72.969 | 1.00 | 0.00 | xxxx | 612 |
| ATOM | 613 | N | GLN | A | 83 | 109.204 | 1.360 | 73.605 | 1.00 | 0.00 | xxxx | 613 |
| ATOM | 614 | CA | GLN | A | 83 | 109.538 | −0.043 | 73.423 | 1.00 | 0.00 | xxxx | 614 |
| ATOM | 615 | C | GLN | A | 83 | 108.786 | −0.651 | 72.231 | 1.00 | 0.00 | xxxx | 615 |
| ATOM | 616 | O | GLN | A | 83 | 108.819 | −1.862 | 72.010 | 1.00 | 0.00 | xxxx | 616 |
| ATOM | 617 | CB | GLN | A | 83 | 109.222 | −0.811 | 74.710 | 1.00 | 0.00 | xxxx | 617 |
| ATOM | 618 | CG | GLN | A | 83 | 110.081 | −2.042 | 74.937 | 1.00 | 0.00 | xxxx | 618 |
| ATOM | 619 | CD | GLN | A | 83 | 110.165 | −2.422 | 76.404 | 1.00 | 0.00 | xxxx | 619 |
| ATOM | 620 | OE1 | GLN | A | 83 | 110.622 | −1.635 | 77.235 | 1.00 | 0.00 | xxxx | 620 |
| ATOM | 621 | NE2 | GLN | A | 83 | 109.712 | −3.626 | 76.730 | 1.00 | 0.00 | xxxx | 621 |
| ATOM | 622 | N | ASN | A | 84 | 108.101 | 0.202 | 71.474 | 1.00 | 0.00 | xxxx | 622 |
| ATOM | 623 | CA | ASN | A | 84 | 107.310 | −0.224 | 70.321 | 1.00 | 0.00 | xxxx | 623 |
| ATOM | 624 | C | ASN | A | 84 | 106.233 | −1.246 | 70.709 | 1.00 | 0.00 | xxxx | 624 |
| ATOM | 625 | O | ASN | A | 84 | 105.928 | −2.157 | 69.939 | 1.00 | 0.00 | xxxx | 625 |
| ATOM | 626 | CB | ASN | A | 84 | 108.221 | −0.799 | 69.228 | 1.00 | 0.00 | xxxx | 626 |
| ATOM | 627 | CG | ASN | A | 84 | 109.380 | 0.118 | 68.896 | 1.00 | 0.00 | xxxx | 627 |
| ATOM | 628 | OD1 | ASN | A | 84 | 109.186 | 1.296 | 68.588 | 1.00 | 0.00 | xxxx | 628 |
| ATOM | 629 | ND2 | ASN | A | 84 | 110.600 | −0.413 | 68.978 | 1.00 | 0.00 | xxxx | 629 |
| ATOM | 630 | N | VAL | A | 85 | 105.647 | −1.074 | 71.891 | 1.00 | 0.00 | xxxx | 630 |
| ATOM | 631 | CA | VAL | A | 85 | 104.676 | −2.033 | 72.419 | 1.00 | 0.00 | xxxx | 631 |
| ATOM | 632 | C | VAL | A | 85 | 103.254 | −1.525 | 72.172 | 1.00 | 0.00 | xxxx | 632 |
| ATOM | 633 | O | VAL | A | 85 | 102.932 | −0.393 | 72.508 | 1.00 | 0.00 | xxxx | 633 |
| ATOM | 634 | CB | VAL | A | 85 | 104.952 | −2.294 | 73.920 | 1.00 | 0.00 | xxxx | 634 |
| ATOM | 635 | CG1 | VAL | A | 85 | 103.787 | −3.044 | 74.592 | 1.00 | 0.00 | xxxx | 635 |
| ATOM | 636 | CG2 | VAL | A | 85 | 106.258 | −3.072 | 74.068 | 1.00 | 0.00 | xxxx | 636 |
| ATOM | 637 | N | PRO | A | 86 | 102.388 | −2.363 | 71.577 | 1.00 | 0.00 | xxxx | 637 |
| ATOM | 638 | CA | PRO | A | 86 | 101.004 | −1.920 | 71.366 | 1.00 | 0.00 | xxxx | 638 |
| ATOM | 639 | C | PRO | A | 86 | 100.209 | −1.854 | 72.666 | 1.00 | 0.00 | xxxx | 639 |
| ATOM | 640 | O | PRO | A | 86 | 100.501 | −2.592 | 73.621 | 1.00 | 0.00 | xxxx | 640 |
| ATOM | 641 | CB | PRO | A | 86 | 100.421 | −2.989 | 70.437 | 1.00 | 0.00 | xxxx | 641 |
| ATOM | 642 | CG | PRO | A | 86 | 101.291 | −4.205 | 70.660 | 1.00 | 0.00 | xxxx | 642 |
| ATOM | 643 | CD | PRO | A | 86 | 102.672 | −3.668 | 70.951 | 1.00 | 0.00 | xxxx | 643 |
| ATOM | 644 | N | VAL | A | 87 | 99.215 | −0.973 | 72.707 | 1.00 | 0.00 | xxxx | 644 |
| ATOM | 645 | CA | VAL | A | 87 | 98.377 | −0.792 | 73.891 | 1.00 | 0.00 | xxxx | 645 |
| ATOM | 646 | C | VAL | A | 87 | 96.907 | −0.896 | 73.513 | 1.00 | 0.00 | xxxx | 646 |
| ATOM | 647 | O | VAL | A | 87 | 96.432 | −0.123 | 72.687 | 1.00 | 0.00 | xxxx | 647 |
| ATOM | 648 | CB | VAL | A | 87 | 98.626 | 0.567 | 74.572 | 1.00 | 0.00 | xxxx | 648 |
| ATOM | 649 | CG1 | VAL | A | 87 | 97.751 | 0.707 | 75.828 | 1.00 | 0.00 | xxxx | 649 |
| ATOM | 650 | CG2 | VAL | A | 87 | 100.070 | 0.724 | 74.941 | 1.00 | 0.00 | xxxx | 650 |
| ATOM | 651 | N | VAL | A | 88 | 96.192 | −1.820 | 74.149 | 1.00 | 0.00 | xxxx | 651 |
| ATOM | 652 | CA | VAL | A | 88 | 94.740 | −1.888 | 74.015 | 1.00 | 0.00 | xxxx | 652 |
| ATOM | 653 | C | VAL | A | 88 | 94.141 | −1.351 | 75.308 | 1.00 | 0.00 | xxxx | 653 |
| ATOM | 654 | O | VAL | A | 88 | 94.129 | −2.049 | 76.335 | 1.00 | 0.00 | xxxx | 654 |
| ATOM | 655 | CB | VAL | A | 88 | 94.255 | −3.317 | 73.725 | 1.00 | 0.00 | xxxx | 655 |
| ATOM | 656 | CG1 | VAL | A | 88 | 92.734 | −3.331 | 73.544 | 1.00 | 0.00 | xxxx | 656 |
| ATOM | 657 | CG2 | VAL | A | 88 | 94.938 | −3.884 | 72.472 | 1.00 | 0.00 | xxxx | 657 |
| ATOM | 658 | N | PHE | A | 89 | 93.667 | −0.108 | 75.279 | 1.00 | 0.00 | xxxx | 658 |
| ATOM | 659 | CA | PHE | A | 89 | 92.946 | 0.445 | 76.426 | 1.00 | 0.00 | xxxx | 659 |
| ATOM | 660 | C | PHE | A | 89 | 91.574 | −0.204 | 76.508 | 1.00 | 0.00 | xxxx | 660 |
| ATOM | 661 | O | PHE | A | 89 | 90.982 | −0.520 | 75.470 | 1.00 | 0.00 | xxxx | 661 |
| ATOM | 662 | CB | PHE | A | 89 | 92.804 | 1.958 | 76.293 | 1.00 | 0.00 | xxxx | 662 |
| ATOM | 663 | CG | PHE | A | 89 | 94.107 | 2.702 | 76.394 | 1.00 | 0.00 | xxxx | 663 |
| ATOM | 664 | CD1 | PHE | A | 89 | 94.708 | 2.899 | 77.625 | 1.00 | 0.00 | xxxx | 664 |
| ATOM | 665 | CD2 | PHE | A | 89 | 94.734 | 3.190 | 75.259 | 1.00 | 0.00 | xxxx | 665 |
| ATOM | 666 | CE1 | PHE | A | 89 | 95.910 | 3.578 | 77.726 | 1.00 | 0.00 | xxxx | 666 |
| ATOM | 667 | CE2 | PHE | A | 89 | 95.936 | 3.879 | 75.355 | 1.00 | 0.00 | xxxx | 667 |
| ATOM | 668 | CZ | PHE | A | 89 | 96.523 | 4.077 | 76.594 | 1.00 | 0.00 | xxxx | 668 |
| ATOM | 669 | N | PHE | A | 90 | 91.048 | −0.413 | 77.712 | 1.00 | 0.00 | xxxx | 669 |
| ATOM | 670 | CA | PHE | A | 90 | 89.680 | −0.944 | 77.785 | 1.00 | 0.00 | xxxx | 670 |
| ATOM | 671 | C | PHE | A | 90 | 88.926 | −0.412 | 78.992 | 1.00 | 0.00 | xxxx | 671 |
| ATOM | 672 | O | PHE | A | 90 | 89.525 | −0.062 | 80.010 | 1.00 | 0.00 | xxxx | 672 |
| ATOM | 673 | CB | PHE | A | 90 | 89.660 | −2.510 | 77.730 | 1.00 | 0.00 | xxxx | 673 |
| ATOM | 674 | CG | PHE | A | 90 | 90.339 | −3.225 | 78.894 | 1.00 | 0.00 | xxxx | 674 |
| ATOM | 675 | CD1 | PHE | A | 90 | 89.573 | −3.927 | 79.824 | 1.00 | 0.00 | xxxx | 675 |
| ATOM | 676 | CD2 | PHE | A | 90 | 91.729 | −3.261 | 79.015 | 1.00 | 0.00 | xxxx | 676 |
| ATOM | 677 | CE1 | PHE | A | 90 | 90.184 | −4.611 | 80.886 | 1.00 | 0.00 | xxxx | 677 |
| ATOM | 678 | CE2 | PHE | A | 90 | 92.349 | −3.934 | 80.071 | 1.00 | 0.00 | xxxx | 678 |
| ATOM | 679 | CZ | PHE | A | 90 | 91.574 | −4.614 | 81.001 | 1.00 | 0.00 | xxxx | 679 |
| ATOM | 680 | N | ASN | A | 91 | 87.616 | −0.279 | 78.791 | 1.00 | 0.00 | xxxx | 680 |
| ATOM | 681 | CA | ASN | A | 91 | 86.590 | 0.085 | 79.786 | 1.00 | 0.00 | xxxx | 681 |
| ATOM | 682 | C | ASN | A | 91 | 86.587 | 1.542 | 80.258 | 1.00 | 0.00 | xxxx | 682 |
| ATOM | 683 | O | ASN | A | 91 | 85.510 | 2.087 | 80.492 | 1.00 | 0.00 | xxxx | 683 |
| ATOM | 684 | CB | ASN | A | 91 | 86.660 | −0.854 | 80.998 | 1.00 | 0.00 | xxxx | 684 |
| ATOM | 685 | CG | ASN | A | 91 | 85.830 | −0.351 | 82.182 | 1.00 | 0.00 | xxxx | 685 |
| ATOM | 686 | OD1 | ASN | A | 91 | 86.370 | 0.228 | 83.120 | 1.00 | 0.00 | xxxx | 686 |
| ATOM | 687 | ND2 | ASN | A | 91 | 84.522 | −0.592 | 82.147 | 1.00 | 0.00 | xxxx | 687 |
| ATOM | 688 | N | LYS | A | 92 | 87.752 | 2.160 | 80.423 | 1.00 | 0.00 | xxxx | 688 |
| ATOM | 689 | CA | LYS | A | 92 | 87.828 | 3.594 | 80.690 | 1.00 | 0.00 | xxxx | 689 |
| ATOM | 690 | C | LYS | A | 92 | 88.391 | 4.250 | 79.446 | 1.00 | 0.00 | xxxx | 690 |
| ATOM | 691 | O | LYS | A | 92 | 89.554 | 4.061 | 79.097 | 1.00 | 0.00 | xxxx | 691 |
| ATOM | 692 | CB | LYS | A | 92 | 88.682 | 3.903 | 81.918 | 1.00 | 0.00 | xxxx | 692 |
| ATOM | 693 | CG | LYS | A | 92 | 88.035 | 3.444 | 83.217 | 1.00 | 0.00 | xxxx | 693 |
| ATOM | 694 | CD | LYS | A | 92 | 88.252 | 4.447 | 84.327 | 1.00 | 0.00 | xxxx | 694 |
| ATOM | 695 | CE | LYS | A | 92 | 88.017 | 3.795 | 85.679 | 1.00 | 0.00 | xxxx | 695 |
| ATOM | 696 | NZ | LYS | A | 92 | 88.338 | 4.731 | 86.803 | 1.00 | 0.00 | xxxx | 696 |
| ATOM | 697 | N | GLU | A | 93 | 87.546 | 5.014 | 78.770 | 1.00 | 0.00 | xxxx | 697 |
| ATOM | 698 | CA | GLU | A | 93 | 87.872 | 5.464 | 77.422 | 1.00 | 0.00 | xxxx | 698 |
| ATOM | 699 | C | GLU | A | 93 | 88.806 | 6.685 | 77.393 | 1.00 | 0.00 | xxxx | 699 |
| ATOM | 700 | O | GLU | A | 93 | 88.495 | 7.729 | 77.986 | 1.00 | 0.00 | xxxx | 700 |
| ATOM | 701 | CB | GLU | A | 93 | 86.585 | 5.765 | 76.656 | 1.00 | 0.00 | xxxx | 701 |
| ATOM | 702 | CG | GLU | A | 93 | 86.840 | 6.151 | 75.212 | 1.00 | 0.00 | xxxx | 702 |
| ATOM | 703 | CD | GLU | A | 93 | 85.572 | 6.254 | 74.396 | 1.00 | 0.00 | xxxx | 703 |
| ATOM | 704 | OE1 | GLU | A | 93 | 84.503 | 5.840 | 74.887 | 1.00 | 0.00 | xxxx | 704 |
| ATOM | 705 | OE2 | GLU | A | 93 | 85.652 | 6.749 | 73.252 | 1.00 | 0.00 | xxxx | 705 |
| ATOM | 706 | N | PRO | A | 94 | 89.950 | 6.563 | 76.701 | 1.00 | 0.00 | xxxx | 706 |
| ATOM | 707 | CA | PRO | A | 94 | 90.860 | 7.705 | 76.521 | 1.00 | 0.00 | xxxx | 707 |
| ATOM | 708 | C | PRO | A | 94 | 90.311 | 8.706 | 75.520 | 1.00 | 0.00 | xxxx | 708 |
| ATOM | 709 | O | PRO | A | 94 | 89.374 | 8.385 | 74.787 | 1.00 | 0.00 | xxxx | 709 |
| ATOM | 710 | CB | PRO | A | 94 | 92.143 | 7.073 | 75.966 | 1.00 | 0.00 | xxxx | 710 |
| ATOM | 711 | CG | PRO | A | 94 | 91.946 | 5.593 | 76.036 | 1.00 | 0.00 | xxxx | 711 |
| ATOM | 712 | CD | PRO | A | 94 | 90.473 | 5.342 | 76.071 | 1.00 | 0.00 | xxxx | 712 |
| ATOM | 713 | N | SER | A | 95 | 90.901 | 9.897 | 75.465 | 1.00 | 0.00 | xxxx | 713 |
| ATOM | 714 | CA | SER | A | 95 | 90.489 | 10.879 | 74.474 | 1.00 | 0.00 | xxxx | 714 |
| ATOM | 715 | C | SER | A | 95 | 90.814 | 10.412 | 73.058 | 1.00 | 0.00 | xxxx | 715 |
| ATOM | 716 | O | SER | A | 95 | 91.716 | 9.616 | 72.837 | 1.00 | 0.00 | xxxx | 716 |
| ATOM | 717 | CB | SER | A | 95 | 91.157 | 12.234 | 74.741 | 1.00 | 0.00 | xxxx | 717 |
| ATOM | 718 | OG | SER | A | 95 | 92.546 | 12.172 | 74.450 | 1.00 | 0.00 | xxxx | 718 |
| ATOM | 719 | N | ARG | A | 96 | 90.052 | 10.923 | 72.100 | 1.00 | 0.00 | xxxx | 719 |
| ATOM | 720 | CA | ARG | A | 96 | 90.334 | 10.701 | 70.685 | 1.00 | 0.00 | xxxx | 720 |
| ATOM | 721 | C | ARG | A | 96 | 91.758 | 11.141 | 70.337 | 1.00 | 0.00 | xxxx | 721 |
| ATOM | 722 | O | ARG | A | 96 | 92.484 | 10.443 | 69.625 | 1.00 | 0.00 | xxxx | 722 |
| ATOM | 723 | CB | ARG | A | 96 | 89.310 | 11.451 | 69.839 | 1.00 | 0.00 | xxxx | 723 |
| ATOM | 724 | CG | ARG | A | 96 | 89.369 | 11.185 | 68.347 | 1.00 | 0.00 | xxxx | 724 |
| ATOM | 725 | CD | ARG | A | 96 | 88.887 | 9.781 | 68.062 | 1.00 | 0.00 | xxxx | 725 |
| ATOM | 726 | NE | ARG | A | 96 | 89.980 | 8.914 | 67.664 | 1.00 | 0.00 | xxxx | 726 |
| ATOM | 727 | CZ | ARG | A | 96 | 89.860 | 7.604 | 67.479 | 1.00 | 0.00 | xxxx | 727 |
| ATOM | 728 | NH1 | ARG | A | 96 | 88.689 | 7.005 | 67.678 | 1.00 | 0.00 | xxxx | 728 |
| ATOM | 729 | NH2 | ARG | A | 96 | 90.914 | 6.900 | 67.099 | 1.00 | 0.00 | xxxx | 729 |
| ATOM | 730 | N | LYS | A | 97 | 92.165 | 12.301 | 70.845 | 1.00 | 0.00 | xxxx | 730 |
| ATOM | 731 | CA | LYS | A | 97 | 93.506 | 12.809 | 70.578 | 1.00 | 0.00 | xxxx | 731 |
| ATOM | 732 | C | LYS | A | 97 | 94.572 | 11.850 | 71.084 | 1.00 | 0.00 | xxxx | 732 |
| ATOM | 733 | O | LYS | A | 97 | 95.597 | 11.648 | 70.428 | 1.00 | 0.00 | xxxx | 733 |
| ATOM | 734 | CB | LYS | A | 97 | 93.707 | 14.180 | 71.227 | 1.00 | 0.00 | xxxx | 734 |
| ATOM | 735 | CG | LYS | A | 97 | 92.951 | 15.304 | 70.545 | 1.00 | 0.00 | xxxx | 735 |
| ATOM | 736 | CD | LYS | A | 97 | 93.178 | 16.635 | 71.271 | 1.00 | 0.00 | xxxx | 736 |
| ATOM | 737 | CE | LYS | A | 97 | 92.407 | 17.759 | 70.595 | 1.00 | 0.00 | xxxx | 737 |
| ATOM | 738 | NZ | LYS | A | 97 | 92.712 | 19.098 | 71.206 | 1.00 | 0.00 | xxxx | 738 |
| ATOM | 739 | N | ALA | A | 98 | 94.344 | 11.277 | 72.264 | 1.00 | 0.00 | xxxx | 739 |
| ATOM | 740 | CA | ALA | A | 98 | 95.304 | 10.315 | 72.803 | 1.00 | 0.00 | xxxx | 740 |
| ATOM | 741 | C | ALA | A | 98 | 95.394 | 9.077 | 71.917 | 1.00 | 0.00 | xxxx | 741 |
| ATOM | 742 | O | ALA | A | 98 | 96.488 | 8.650 | 71.553 | 1.00 | 0.00 | xxxx | 742 |
| ATOM | 743 | CB | ALA | A | 98 | 94.942 | 9.925 | 74.237 | 1.00 | 0.00 | xxxx | 743 |
| ATOM | 744 | N | LEU | A | 99 | 94.246 | 8.518 | 71.551 | 1.00 | 0.00 | xxxx | 744 |
| ATOM | 745 | CA | LEU | A | 99 | 94.233 | 7.346 | 70.673 | 1.00 | 0.00 | xxxx | 745 |
| ATOM | 746 | C | LEU | A | 99 | 94.941 | 7.608 | 69.346 | 1.00 | 0.00 | xxxx | 746 |
| ATOM | 747 | O | LEU | A | 99 | 95.665 | 6.754 | 68.837 | 1.00 | 0.00 | xxxx | 747 |
| ATOM | 748 | CB | LEU | A | 99 | 92.801 | 6.895 | 70.397 | 1.00 | 0.00 | xxxx | 748 |
| ATOM | 749 | CG | LEU | A | 99 | 92.063 | 6.297 | 71.584 | 1.00 | 0.00 | xxxx | 749 |
| ATOM | 750 | CD1 | LEU | A | 99 | 90.656 | 5.872 | 71.158 | 1.00 | 0.00 | xxxx | 750 |
| ATOM | 751 | CD2 | LEU | A | 99 | 92.829 | 5.105 | 72.167 | 1.00 | 0.00 | xxxx | 751 |
| ATOM | 752 | N | ASP | A | 100 | 94.725 | 8.790 | 68.779 | 1.00 | 0.00 | xxxx | 752 |
| ATOM | 753 | CA | ASP | A | 100 | 95.310 | 9.129 | 67.489 | 1.00 | 0.00 | xxxx | 753 |
| ATOM | 754 | C | ASP | A | 100 | 96.808 | 9.421 | 67.547 | 1.00 | 0.00 | xxxx | 754 |
| ATOM | 755 | O | ASP | A | 100 | 97.473 | 9.473 | 66.508 | 1.00 | 0.00 | xxxx | 755 |
| ATOM | 756 | CB | ASP | A | 100 | 94.579 | 10.332 | 66.887 | 1.00 | 0.00 | xxxx | 756 |
| ATOM | 757 | CG | ASP | A | 100 | 93.189 | 9.971 | 66.389 | 1.00 | 0.00 | xxxx | 757 |
| ATOM | 758 | OD1 | ASP | A | 100 | 92.927 | 8.762 | 66.198 | 1.00 | 0.00 | xxxx | 758 |
| ATOM | 759 | OD2 | ASP | A | 100 | 92.362 | 10.887 | 66.199 | 1.00 | 0.00 | xxxx | 759 |
| ATOM | 760 | N | SER | A | 101 | 97.343 | 9.599 | 68.750 | 1.00 | 0.00 | xxxx | 760 |
| ATOM | 761 | CA | SER | A | 101 | 98.739 | 9.990 | 68.902 | 1.00 | 0.00 | xxxx | 761 |
| ATOM | 762 | C | SER | A | 101 | 99.714 | 8.818 | 68.752 | 1.00 | 0.00 | xxxx | 762 |
| ATOM | 763 | O | SER | A | 101 | 100.925 | 9.023 | 68.654 | 1.00 | 0.00 | xxxx | 763 |
| ATOM | 764 | CB | SER | A | 101 | 98.958 | 10.666 | 70.263 | 1.00 | 0.00 | xxxx | 764 |
| ATOM | 765 | OG | SER | A | 101 | 98.904 | 9.725 | 71.329 | 1.00 | 0.00 | xxxx | 765 |
| ATOM | 766 | N | TYR | A | 102 | 99.187 | 7.595 | 68.740 | 1.00 | 0.00 | xxxx | 766 |
| ATOM | 767 | CA | TYR | A | 102 | 100.022 | 6.395 | 68.694 | 1.00 | 0.00 | xxxx | 767 |
| ATOM | 768 | C | TYR | A | 102 | 99.397 | 5.398 | 67.737 | 1.00 | 0.00 | xxxx | 768 |
| ATOM | 769 | O | TYR | A | 102 | 98.228 | 5.037 | 67.906 | 1.00 | 0.00 | xxxx | 769 |
| ATOM | 770 | CB | TYR | A | 102 | 100.154 | 5.792 | 70.096 | 1.00 | 0.00 | xxxx | 770 |
| ATOM | 771 | CG | TYR | A | 102 | 101.138 | 4.640 | 70.265 | 1.00 | 0.00 | xxxx | 771 |
| ATOM | 772 | CD1 | TYR | A | 102 | 100.853 | 3.593 | 71.138 | 1.00 | 0.00 | xxxx | 772 |
| ATOM | 773 | CD2 | TYR | A | 102 | 102.352 | 4.614 | 69.596 | 1.00 | 0.00 | xxxx | 773 |
| ATOM | 774 | CE1 | TYR | A | 102 | 101.740 | 2.541 | 71.330 | 1.00 | 0.00 | xxxx | 774 |
| ATOM | 775 | CE2 | TYR | A | 102 | 103.260 | 3.563 | 69.780 | 1.00 | 0.00 | xxxx | 775 |
| ATOM | 776 | CZ | TYR | A | 102 | 102.941 | 2.531 | 70.660 | 1.00 | 0.00 | xxxx | 776 |
| ATOM | 777 | OH | TYR | A | 102 | 103.832 | 1.493 | 70.862 | 1.00 | 0.00 | xxxx | 777 |
| ATOM | 778 | N | ASP | A | 103 | 100.165 | 4.951 | 66.739 | 1.00 | 0.00 | xxxx | 778 |
| ATOM | 779 | CA | ASP | A | 103 | 99.602 | 4.085 | 65.707 | 1.00 | 0.00 | xxxx | 779 |
| ATOM | 780 | C | ASP | A | 103 | 99.207 | 2.719 | 66.273 | 1.00 | 0.00 | xxxx | 780 |
| ATOM | 781 | O | ASP | A | 103 | 98.417 | 2.008 | 65.670 | 1.00 | 0.00 | xxxx | 781 |
| ATOM | 782 | CB | ASP | A | 103 | 100.573 | 3.912 | 64.519 | 1.00 | 0.00 | xxxx | 782 |
| ATOM | 783 | CG | ASP | A | 103 | 101.943 | 3.400 | 64.934 | 1.00 | 0.00 | xxxx | 783 |
| ATOM | 784 | OD1 | ASP | A | 103 | 102.138 | 3.062 | 66.120 | 1.00 | 0.00 | xxxx | 784 |
| ATOM | 785 | OD2 | ASP | A | 103 | 102.834 | 3.328 | 64.057 | 1.00 | 0.00 | xxxx | 785 |
| ATOM | 786 | N | LYS | A | 104 | 99.732 | 2.361 | 67.440 | 1.00 | 0.00 | xxxx | 786 |
| ATOM | 787 | CA | LYS | A | 104 | 99.415 | 1.066 | 68.020 | 1.00 | 0.00 | xxxx | 787 |
| ATOM | 788 | C | LYS | A | 104 | 98.553 | 1.168 | 69.286 | 1.00 | 0.00 | xxxx | 788 |
| ATOM | 789 | O | LYS | A | 104 | 98.544 | 0.235 | 70.094 | 1.00 | 0.00 | xxxx | 789 |
| ATOM | 790 | CB | LYS | A | 104 | 100.710 | 0.295 | 68.321 | 1.00 | 0.00 | xxxx | 790 |
| ATOM | 791 | CG | LYS | A | 104 | 101.487 | −0.125 | 67.063 | 1.00 | 0.00 | xxxx | 791 |
| ATOM | 792 | CD | LYS | A | 104 | 102.803 | −0.812 | 67.409 | 1.00 | 0.00 | xxxx | 792 |
| ATOM | 793 | CE | LYS | A | 104 | 103.833 | 0.175 | 67.931 | 1.00 | 0.00 | xxxx | 793 |
| ATOM | 794 | NZ | LYS | A | 104 | 104.492 | 0.950 | 66.837 | 1.00 | 0.00 | xxxx | 794 |
| ATOM | 795 | N | ALA | A | 105 | 97.822 | 2.277 | 69.446 | 1.00 | 0.00 | xxxx | 795 |
| ATOM | 796 | CA | ALA | A | 105 | 96.825 | 2.410 | 70.515 | 1.00 | 0.00 | xxxx | 796 |
| ATOM | 797 | C | ALA | A | 105 | 95.419 | 2.078 | 70.019 | 1.00 | 0.00 | xxxx | 797 |
| ATOM | 798 | O | ALA | A | 105 | 95.012 | 2.542 | 68.957 | 1.00 | 0.00 | xxxx | 798 |
| ATOM | 799 | CB | ALA | A | 105 | 96.852 | 3.822 | 71.107 | 1.00 | 0.00 | xxxx | 799 |
| ATOM | 800 | N | TYR | A | 106 | 94.685 | 1.279 | 70.797 | 1.00 | 0.00 | xxxx | 800 |
| ATOM | 801 | CA | TYR | A | 106 | 93.312 | 0.880 | 70.483 | 1.00 | 0.00 | xxxx | 801 |
| ATOM | 802 | C | TYR | A | 106 | 92.440 | 1.016 | 71.729 | 1.00 | 0.00 | xxxx | 802 |
| ATOM | 803 | O | TYR | A | 106 | 92.966 | 1.198 | 72.826 | 1.00 | 0.00 | xxxx | 803 |
| ATOM | 804 | CB | TYR | A | 106 | 93.268 | −0.567 | 69.975 | 1.00 | 0.00 | xxxx | 804 |
| ATOM | 805 | CG | TYR | A | 106 | 93.943 | −0.752 | 68.640 | 1.00 | 0.00 | xxxx | 805 |
| ATOM | 806 | CD1 | TYR | A | 106 | 93.197 | −0.796 | 67.479 | 1.00 | 0.00 | xxxx | 806 |
| ATOM | 807 | CD2 | TYR | A | 106 | 95.323 | −0.852 | 68.546 | 1.00 | 0.00 | xxxx | 807 |
| ATOM | 808 | CE1 | TYR | A | 106 | 93.807 | −0.942 | 66.245 | 1.00 | 0.00 | xxxx | 808 |
| ATOM | 809 | CE2 | TYR | A | 106 | 95.945 | −1.003 | 67.320 | 1.00 | 0.00 | xxxx | 809 |
| ATOM | 810 | CZ | TYR | A | 106 | 95.180 | −1.045 | 66.181 | 1.00 | 0.00 | xxxx | 810 |
| ATOM | 811 | OH | TYR | A | 106 | 95.776 | −1.190 | 64.941 | 1.00 | 0.00 | xxxx | 811 |
| ATOM | 812 | N | TYR | A | 107 | 91.120 | 0.911 | 71.562 | 1.00 | 0.00 | xxxx | 812 |
| ATOM | 813 | CA | TYR | A | 107 | 90.212 | 0.931 | 72.709 | 1.00 | 0.00 | xxxx | 813 |
| ATOM | 814 | C | TYR | A | 107 | 89.090 | −0.082 | 72.540 | 1.00 | 0.00 | xxxx | 814 |
| ATOM | 815 | O | TYR | A | 107 | 88.493 | −0.173 | 71.477 | 1.00 | 0.00 | xxxx | 815 |
| ATOM | 816 | CB | TYR | A | 107 | 89.587 | 2.325 | 72.927 | 1.00 | 0.00 | xxxx | 816 |
| ATOM | 817 | CG | TYR | A | 107 | 88.629 | 2.324 | 74.109 | 1.00 | 0.00 | xxxx | 817 |
| ATOM | 818 | CD1 | TYR | A | 107 | 89.110 | 2.165 | 75.405 | 1.00 | 0.00 | xxxx | 818 |
| ATOM | 819 | CD2 | TYR | A | 107 | 87.255 | 2.445 | 73.935 | 1.00 | 0.00 | xxxx | 819 |
| ATOM | 820 | CE1 | TYR | A | 107 | 88.257 | 2.135 | 76.488 | 1.00 | 0.00 | xxxx | 820 |
| ATOM | 821 | CE2 | TYR | A | 107 | 86.387 | 2.414 | 75.024 | 1.00 | 0.00 | xxxx | 821 |
| ATOM | 822 | CZ | TYR | A | 107 | 86.906 | 2.261 | 76.297 | 1.00 | 0.00 | xxxx | 822 |
| ATOM | 823 | OH | TYR | A | 107 | 86.058 | 2.217 | 77.383 | 1.00 | 0.00 | xxxx | 823 |
| ATOM | 824 | N | VAL | A | 108 | 88.797 | −0.817 | 73.612 | 1.00 | 0.00 | xxxx | 824 |
| ATOM | 825 | CA | VAL | A | 108 | 87.650 | −1.721 | 73.661 | 1.00 | 0.00 | xxxx | 825 |
| ATOM | 826 | C | VAL | A | 108 | 86.720 | −1.294 | 74.784 | 1.00 | 0.00 | xxxx | 826 |
| ATOM | 827 | O | VAL | A | 108 | 87.141 | −1.184 | 75.934 | 1.00 | 0.00 | xxxx | 827 |
| ATOM | 828 | CB | VAL | A | 108 | 88.087 | −3.174 | 73.877 | 1.00 | 0.00 | xxxx | 828 |
| ATOM | 829 | CG1 | VAL | A | 108 | 86.848 | −4.086 | 74.051 | 1.00 | 0.00 | xxxx | 829 |
| ATOM | 830 | CG2 | VAL | A | 108 | 88.972 | −3.674 | 72.718 | 1.00 | 0.00 | xxxx | 830 |
| ATOM | 831 | N | GLY | A | 109 | 85.460 | −1.060 | 74.457 | 1.00 | 0.00 | xxxx | 831 |
| ATOM | 832 | CA | GLY | A | 109 | 84.492 | −0.672 | 75.470 | 1.00 | 0.00 | xxxx | 832 |
| ATOM | 833 | C | GLY | A | 109 | 83.079 | −0.729 | 74.934 | 1.00 | 0.00 | xxxx | 833 |
| ATOM | 834 | O | GLY | A | 109 | 82.682 | −1.717 | 74.321 | 1.00 | 0.00 | xxxx | 834 |
| ATOM | 835 | N | THR | A | 110 | 82.328 | 0.335 | 75.196 | 1.00 | 0.00 | xxxx | 835 |
| ATOM | 836 | CA | THR | A | 110 | 80.915 | 0.410 | 74.843 | 1.00 | 0.00 | xxxx | 836 |
| ATOM | 837 | C | THR | A | 110 | 80.556 | 1.792 | 74.323 | 1.00 | 0.00 | xxxx | 837 |
| ATOM | 838 | O | THR | A | 110 | 81.343 | 2.734 | 74.442 | 1.00 | 0.00 | xxxx | 838 |
| ATOM | 839 | CB | THR | A | 110 | 80.007 | 0.132 | 76.056 | 1.00 | 0.00 | xxxx | 839 |
| ATOM | 840 | OG1 | THR | A | 110 | 80.096 | 1.231 | 76.975 | 1.00 | 0.00 | xxxx | 840 |
| ATOM | 841 | CG2 | THR | A | 110 | 80.418 | −1.153 | 76.784 | 1.00 | 0.00 | xxxx | 841 |
| ATOM | 842 | N | ASP | A | 111 | 79.354 | 1.912 | 73.755 | 1.00 | 0.00 | xxxx | 842 |
| ATOM | 843 | CA | ASP | A | 111 | 78.733 | 3.209 | 73.500 | 1.00 | 0.00 | xxxx | 843 |
| ATOM | 844 | C | ASP | A | 111 | 77.968 | 3.609 | 74.757 | 1.00 | 0.00 | xxxx | 844 |
| ATOM | 845 | O | ASP | A | 111 | 76.852 | 3.152 | 74.977 | 1.00 | 0.00 | xxxx | 845 |
| ATOM | 846 | CB | ASP | A | 111 | 77.803 | 3.122 | 72.295 | 1.00 | 0.00 | xxxx | 846 |
| ATOM | 847 | CG | ASP | A | 111 | 77.151 | 4.443 | 71.945 | 1.00 | 0.00 | xxxx | 847 |
| ATOM | 848 | OD1 | ASP | A | 111 | 77.546 | 5.492 | 72.494 | 1.00 | 0.00 | xxxx | 848 |
| ATOM | 849 | OD2 | ASP | A | 111 | 76.245 | 4.423 | 71.089 | 1.00 | 0.00 | xxxx | 849 |
| ATOM | 850 | N | SER | A | 112 | 78.584 | 4.441 | 75.593 | 1.00 | 0.00 | xxxx | 850 |
| ATOM | 851 | CA | SER | A | 112 | 78.050 | 4.710 | 76.928 | 1.00 | 0.00 | xxxx | 851 |
| ATOM | 852 | C | SER | A | 112 | 76.588 | 5.181 | 76.924 | 1.00 | 0.00 | xxxx | 852 |
| ATOM | 853 | O | SER | A | 112 | 75.783 | 4.735 | 77.743 | 1.00 | 0.00 | xxxx | 853 |
| ATOM | 854 | CB | SER | A | 112 | 78.938 | 5.743 | 77.632 | 1.00 | 0.00 | xxxx | 854 |
| ATOM | 855 | OG | SER | A | 112 | 80.282 | 5.273 | 77.722 | 1.00 | 0.00 | xxxx | 855 |
| ATOM | 856 | N | LYS | A | 113 | 76.241 | 6.053 | 75.980 | 1.00 | 0.00 | xxxx | 856 |
| ATOM | 857 | CA | LYS | A | 113 | 74.895 | 6.620 | 75.943 | 1.00 | 0.00 | xxxx | 857 |
| ATOM | 858 | C | LYS | A | 113 | 73.836 | 5.534 | 75.701 | 1.00 | 0.00 | xxxx | 858 |
| ATOM | 859 | O | LYS | A | 113 | 72.714 | 5.640 | 76.197 | 1.00 | 0.00 | xxxx | 859 |
| ATOM | 860 | CB | LYS | A | 113 | 74.799 | 7.711 | 74.877 | 1.00 | 0.00 | xxxx | 860 |
| ATOM | 861 | CG | LYS | A | 113 | 74.780 | 7.203 | 73.457 | 1.00 | 0.00 | xxxx | 861 |
| ATOM | 862 | CD | LYS | A | 113 | 74.286 | 8.278 | 72.499 | 1.00 | 0.00 | xxxx | 862 |
| ATOM | 863 | CE | LYS | A | 113 | 74.040 | 7.701 | 71.113 | 1.00 | 0.00 | xxxx | 863 |
| ATOM | 864 | NZ | LYS | A | 113 | 75.238 | 6.994 | 70.575 | 1.00 | 0.00 | xxxx | 864 |
| ATOM | 865 | N | GLU | A | 114 | 74.207 | 4.479 | 74.975 | 1.00 | 0.00 | xxxx | 865 |
| ATOM | 866 | CA | GLU | A | 114 | 73.270 | 3.394 | 74.699 | 1.00 | 0.00 | xxxx | 866 |
| ATOM | 867 | C | GLU | A | 114 | 72.725 | 2.781 | 75.978 | 1.00 | 0.00 | xxxx | 867 |
| ATOM | 868 | O | GLU | A | 114 | 71.543 | 2.448 | 76.065 | 1.00 | 0.00 | xxxx | 868 |
| ATOM | 869 | CB | GLU | A | 114 | 73.926 | 2.311 | 73.843 | 1.00 | 0.00 | xxxx | 869 |
| ATOM | 870 | CG | GLU | A | 114 | 72.990 | 1.167 | 73.486 | 1.00 | 0.00 | xxxx | 870 |
| ATOM | 871 | CD | GLU | A | 114 | 73.671 | 0.061 | 72.706 | 1.00 | 0.00 | xxxx | 871 |
| ATOM | 872 | OE1 | GLU | A | 114 | 74.705 | −0.457 | 73.172 | 1.00 | 0.00 | xxxx | 872 |
| ATOM | 873 | OE2 | GLU | A | 114 | 73.176 | −0.290 | 71.615 | 1.00 | 0.00 | xxxx | 873 |
| ATOM | 874 | N | SER | A | 115 | 73.574 | 2.631 | 76.987 | 1.00 | 0.00 | xxxx | 874 |
| ATOM | 875 | CA | SER | A | 115 | 73.103 | 2.025 | 78.228 | 1.00 | 0.00 | xxxx | 875 |
| ATOM | 876 | C | SER | A | 115 | 72.096 | 2.924 | 78.953 | 1.00 | 0.00 | xxxx | 876 |
| ATOM | 877 | O | SER | A | 115 | 71.162 | 2.423 | 79.559 | 1.00 | 0.00 | xxxx | 877 |
| ATOM | 878 | CB | SER | A | 115 | 74.274 | 1.673 | 79.162 | 1.00 | 0.00 | xxxx | 878 |
| ATOM | 879 | OG | SER | A | 115 | 75.073 | 2.791 | 79.501 | 1.00 | 0.00 | xxxx | 879 |
| ATOM | 880 | N | GLY | A | 116 | 72.282 | 4.240 | 78.880 | 1.00 | 0.00 | xxxx | 880 |
| ATOM | 881 | CA | GLY | A | 116 | 71.305 | 5.160 | 79.446 | 1.00 | 0.00 | xxxx | 881 |
| ATOM | 882 | C | GLY | A | 116 | 69.965 | 5.122 | 78.724 | 1.00 | 0.00 | xxxx | 882 |
| ATOM | 883 | O | GLY | A | 116 | 68.903 | 5.120 | 79.354 | 1.00 | 0.00 | xxxx | 883 |
| ATOM | 884 | N | ILE | A | 117 | 70.015 | 5.109 | 77.396 | 1.00 | 0.00 | xxxx | 884 |
| ATOM | 885 | CA | ILE | A | 117 | 68.807 | 5.008 | 76.584 | 1.00 | 0.00 | xxxx | 885 |
| ATOM | 886 | C | ILE | A | 117 | 68.071 | 3.700 | 76.894 | 1.00 | 0.00 | xxxx | 886 |
| ATOM | 887 | O | ILE | A | 117 | 66.856 | 3.699 | 77.111 | 1.00 | 0.00 | xxxx | 887 |
| ATOM | 888 | CB | ILE | A | 117 | 69.147 | 5.119 | 75.093 | 1.00 | 0.00 | xxxx | 888 |
| ATOM | 889 | CG2 | ILE | A | 117 | 67.908 | 4.856 | 74.240 | 1.00 | 0.00 | xxxx | 889 |
| ATOM | 890 | CG1 | ILE | A | 117 | 69.742 | 6.498 | 74.792 | 1.00 | 0.00 | xxxx | 890 |
| ATOM | 891 | CD1 | ILE | A | 117 | 70.257 | 6.656 | 73.391 | 1.00 | 0.00 | xxxx | 891 |
| ATOM | 892 | N | ILE | A | 118 | 68.800 | 2.586 | 76.914 | 1.00 | 0.00 | xxxx | 892 |
| ATOM | 893 | CA | ILE | A | 118 | 68.164 | 1.304 | 77.208 | 1.00 | 0.00 | xxxx | 893 |
| ATOM | 894 | C | ILE | A | 118 | 67.565 | 1.302 | 78.609 | 1.00 | 0.00 | xxxx | 894 |
| ATOM | 895 | O | ILE | A | 118 | 66.458 | 0.799 | 78.810 | 1.00 | 0.00 | xxxx | 895 |
| ATOM | 896 | CB | ILE | A | 118 | 69.142 | 0.147 | 77.015 | 1.00 | 0.00 | xxxx | 896 |
| ATOM | 897 | CG1 | ILE | A | 118 | 69.483 | 0.027 | 75.528 | 1.00 | 0.00 | xxxx | 897 |
| ATOM | 898 | CG2 | ILE | A | 118 | 68.535 | −1.155 | 77.531 | 1.00 | 0.00 | xxxx | 898 |
| ATOM | 899 | CD1 | ILE | A | 118 | 70.562 | −1.000 | 75.227 | 1.00 | 0.00 | xxxx | 899 |
| ATOM | 900 | N | GLN | A | 119 | 68.272 | 1.879 | 79.579 | 1.00 | 0.00 | xxxx | 900 |
| ATOM | 901 | CA | GLN | A | 119 | 67.736 | 1.916 | 80.932 | 1.00 | 0.00 | xxxx | 901 |
| ATOM | 902 | C | GLN | A | 119 | 66.448 | 2.738 | 80.996 | 1.00 | 0.00 | xxxx | 902 |
| ATOM | 903 | O | GLN | A | 119 | 65.490 | 2.339 | 81.649 | 1.00 | 0.00 | xxxx | 903 |
| ATOM | 904 | CB | GLN | A | 119 | 68.773 | 2.472 | 81.915 | 1.00 | 0.00 | xxxx | 904 |
| ATOM | 905 | CG | GLN | A | 119 | 68.356 | 2.257 | 83.375 | 1.00 | 0.00 | xxxx | 905 |
| ATOM | 906 | CD | GLN | A | 119 | 69.334 | 2.890 | 84.345 | 1.00 | 0.00 | xxxx | 906 |
| ATOM | 907 | OE1 | GLN | A | 119 | 69.729 | 4.042 | 84.172 | 1.00 | 0.00 | xxxx | 907 |
| ATOM | 908 | NE2 | GLN | A | 119 | 69.722 | 2.145 | 85.371 | 1.00 | 0.00 | xxxx | 908 |
| ATOM | 909 | N | GLY | A | 120 | 66.422 | 3.866 | 80.296 | 1.00 | 0.00 | xxxx | 909 |
| ATOM | 910 | CA | GLY | A | 120 | 65.229 | 4.697 | 80.240 | 1.00 | 0.00 | xxxx | 910 |
| ATOM | 911 | C | GLY | A | 120 | 64.057 | 3.986 | 79.584 | 1.00 | 0.00 | xxxx | 911 |
| ATOM | 912 | O | GLY | A | 120 | 62.917 | 4.111 | 80.032 | 1.00 | 0.00 | xxxx | 912 |
| ATOM | 913 | N | ASP | A | 121 | 64.323 | 3.268 | 78.502 | 1.00 | 0.00 | xxxx | 913 |
| ATOM | 914 | CA | ASP | A | 121 | 63.258 | 2.583 | 77.780 | 1.00 | 0.00 | xxxx | 914 |
| ATOM | 915 | C | ASP | A | 121 | 62.695 | 1.453 | 78.634 | 1.00 | 0.00 | xxxx | 915 |
| ATOM | 916 | O | ASP | A | 121 | 61.505 | 1.142 | 78.573 | 1.00 | 0.00 | xxxx | 916 |
| ATOM | 917 | CB | ASP | A | 121 | 63.779 | 2.051 | 76.446 | 1.00 | 0.00 | xxxx | 917 |
| ATOM | 918 | CG | ASP | A | 121 | 62.776 | 1.169 | 75.740 | 1.00 | 0.00 | xxxx | 918 |
| ATOM | 919 | OD1 | ASP | A | 121 | 61.896 | 1.712 | 75.040 | 1.00 | 0.00 | xxxx | 919 |
| ATOM | 920 | OD2 | ASP | A | 121 | 62.875 | −0.068 | 75.881 | 1.00 | 0.00 | xxxx | 920 |
| ATOM | 921 | N | LEU | A | 122 | 63.564 | 0.857 | 79.444 | 1.00 | 0.00 | xxxx | 921 |
| ATOM | 922 | CA | LEU | A | 122 | 63.166 | −0.201 | 80.362 | 1.00 | 0.00 | xxxx | 922 |
| ATOM | 923 | C | LEU | A | 122 | 62.257 | 0.340 | 81.468 | 1.00 | 0.00 | xxxx | 923 |
| ATOM | 924 | O | LEU | A | 122 | 61.190 | −0.218 | 81.734 | 1.00 | 0.00 | xxxx | 924 |
| ATOM | 925 | CB | LEU | A | 122 | 64.407 | −0.866 | 80.964 | 1.00 | 0.00 | xxxx | 925 |
| ATOM | 926 | CG | LEU | A | 122 | 64.182 | −2.202 | 81.667 | 1.00 | 0.00 | xxxx | 926 |
| ATOM | 927 | CD1 | LEU | A | 122 | 63.510 | −3.183 | 80.713 | 1.00 | 0.00 | xxxx | 927 |
| ATOM | 928 | CD2 | LEU | A | 122 | 65.509 | −2.750 | 82.177 | 1.00 | 0.00 | xxxx | 928 |
| ATOM | 929 | N | ILE | A | 123 | 62.684 | 1.429 | 82.108 | 1.00 | 0.00 | xxxx | 929 |
| ATOM | 930 | CA | ILE | A | 123 | 61.890 | 2.097 | 83.132 | 1.00 | 0.00 | xxxx | 930 |
| ATOM | 931 | C | ILE | A | 123 | 60.524 | 2.474 | 82.561 | 1.00 | 0.00 | xxxx | 931 |
| ATOM | 932 | O | ILE | A | 123 | 59.499 | 2.260 | 83.206 | 1.00 | 0.00 | xxxx | 932 |
| ATOM | 933 | CB | ILE | A | 123 | 62.622 | 3.325 | 83.692 | 1.00 | 0.00 | xxxx | 933 |
| ATOM | 934 | CG1 | ILE | A | 123 | 63.881 | 2.881 | 84.437 | 1.00 | 0.00 | xxxx | 934 |
| ATOM | 935 | CG2 | ILE | A | 123 | 61.692 | 4.122 | 84.624 | 1.00 | 0.00 | xxxx | 935 |
| ATOM | 936 | CD1 | ILE | A | 123 | 64.782 | 4.037 | 84.868 | 1.00 | 0.00 | xxxx | 936 |
| ATOM | 937 | N | ALA | A | 124 | 60.501 | 3.010 | 81.346 | 1.00 | 0.00 | xxxx | 937 |
| ATOM | 938 | CA | ALA | A | 124 | 59.229 | 3.381 | 80.715 | 1.00 | 0.00 | xxxx | 938 |
| ATOM | 939 | C | ALA | A | 124 | 58.288 | 2.187 | 80.553 | 1.00 | 0.00 | xxxx | 939 |
| ATOM | 940 | O | ALA | A | 124 | 57.078 | 2.294 | 80.797 | 1.00 | 0.00 | xxxx | 940 |
| ATOM | 941 | CB | ALA | A | 124 | 59.483 | 4.037 | 79.367 | 1.00 | 0.00 | xxxx | 941 |
| ATOM | 942 | N | LYS | A | 125 | 58.845 | 1.052 | 80.141 | 1.00 | 0.00 | xxxx | 942 |
| ATOM | 943 | CA | LYS | A | 125 | 58.071 | −0.168 | 79.945 | 1.00 | 0.00 | xxxx | 943 |
| ATOM | 944 | C | LYS | A | 125 | 57.428 | −0.619 | 81.251 | 1.00 | 0.00 | xxxx | 944 |
| ATOM | 945 | O | LYS | A | 125 | 56.235 | −0.954 | 81.296 | 1.00 | 0.00 | xxxx | 945 |
| ATOM | 946 | CB | LYS | A | 125 | 58.978 | −1.267 | 79.381 | 1.00 | 0.00 | xxxx | 946 |
| ATOM | 947 | CG | LYS | A | 125 | 58.334 | −2.651 | 79.292 | 1.00 | 0.00 | xxxx | 947 |
| ATOM | 948 | CD | LYS | A | 125 | 59.409 | −3.705 | 79.021 | 1.00 | 0.00 | xxxx | 948 |
| ATOM | 949 | CE | LYS | A | 125 | 58.828 | −5.103 | 79.036 | 1.00 | 0.00 | xxxx | 949 |
| ATOM | 950 | NZ | LYS | A | 125 | 59.892 | −6.128 | 78.877 | 1.00 | 0.00 | xxxx | 950 |
| ATOM | 951 | N | HIS | A | 126 | 58.221 | −0.622 | 82.320 | 1.00 | 0.00 | xxxx | 951 |
| ATOM | 952 | CA | HIS | A | 126 | 57.741 | −1.131 | 83.596 | 1.00 | 0.00 | xxxx | 952 |
| ATOM | 953 | C | HIS | A | 126 | 56.831 | −0.138 | 84.296 | 1.00 | 0.00 | xxxx | 953 |
| ATOM | 954 | O | HIS | A | 126 | 55.894 | −0.543 | 84.980 | 1.00 | 0.00 | xxxx | 954 |
| ATOM | 955 | CB | HIS | A | 126 | 58.922 | −1.523 | 84.478 | 1.00 | 0.00 | xxxx | 955 |
| ATOM | 956 | CG | HIS | A | 126 | 59.593 | −2.778 | 84.016 | 1.00 | 0.00 | xxxx | 956 |
| ATOM | 957 | ND1 | HIS | A | 126 | 59.266 | −4.020 | 84.514 | 1.00 | 0.00 | xxxx | 957 |
| ATOM | 958 | CD2 | HIS | A | 126 | 60.510 | −2.990 | 83.041 | 1.00 | 0.00 | xxxx | 958 |
| ATOM | 959 | CE1 | HIS | A | 126 | 59.983 | −4.943 | 83.893 | 1.00 | 0.00 | xxxx | 959 |
| ATOM | 960 | NE2 | HIS | A | 126 | 60.744 | −4.343 | 82.995 | 1.00 | 0.00 | xxxx | 960 |
| ATOM | 961 | N | TRP | A | 127 | 57.089 | 1.154 | 84.103 | 1.00 | 0.00 | xxxx | 961 |
| ATOM | 962 | CA | TRP | A | 127 | 56.206 | 2.213 | 84.605 | 1.00 | 0.00 | xxxx | 962 |
| ATOM | 963 | C | TRP | A | 127 | 54.805 | 2.083 | 84.022 | 1.00 | 0.00 | xxxx | 963 |
| ATOM | 964 | O | TRP | A | 127 | 53.803 | 2.171 | 84.746 | 1.00 | 0.00 | xxxx | 964 |
| ATOM | 965 | CB | TRP | A | 127 | 56.807 | 3.583 | 84.262 | 1.00 | 0.00 | xxxx | 965 |
| ATOM | 966 | CG | TRP | A | 127 | 56.008 | 4.805 | 84.674 | 1.00 | 0.00 | xxxx | 966 |
| ATOM | 967 | CD1 | TRP | A | 127 | 55.272 | 5.607 | 83.854 | 1.00 | 0.00 | xxxx | 967 |
| ATOM | 968 | CD2 | TRP | A | 127 | 55.927 | 5.383 | 85.982 | 1.00 | 0.00 | xxxx | 968 |
| ATOM | 969 | NE1 | TRP | A | 127 | 54.716 | 6.642 | 84.574 | 1.00 | 0.00 | xxxx | 969 |
| ATOM | 970 | CE2 | TRP | A | 127 | 55.110 | 6.527 | 85.882 | 1.00 | 0.00 | xxxx | 970 |
| ATOM | 971 | CE3 | TRP | A | 127 | 56.469 | 5.045 | 87.229 | 1.00 | 0.00 | xxxx | 971 |
| ATOM | 972 | CZ2 | TRP | A | 127 | 54.818 | 7.335 | 86.982 | 1.00 | 0.00 | xxxx | 972 |
| ATOM | 973 | CZ3 | TRP | A | 127 | 56.183 | 5.849 | 88.320 | 1.00 | 0.00 | xxxx | 973 |
| ATOM | 974 | CH2 | TRP | A | 127 | 55.360 | 6.980 | 88.191 | 1.00 | 0.00 | xxxx | 974 |
| ATOM | 975 | N | ALA | A | 128 | 54.730 | 1.869 | 82.713 | 1.00 | 0.00 | xxxx | 975 |
| ATOM | 976 | CA | ALA | A | 128 | 53.439 | 1.684 | 82.051 | 1.00 | 0.00 | xxxx | 976 |
| ATOM | 977 | C | ALA | A | 128 | 52.691 | 0.465 | 82.606 | 1.00 | 0.00 | xxxx | 977 |
| ATOM | 978 | O | ALA | A | 128 | 51.459 | 0.480 | 82.734 | 1.00 | 0.00 | xxxx | 978 |
| ATOM | 979 | CB | ALA | A | 128 | 53.635 | 1.546 | 80.545 | 1.00 | 0.00 | xxxx | 979 |
| ATOM | 980 | N | ALA | A | 129 | 53.438 | −0.580 | 82.946 | 1.00 | 0.00 | xxxx | 980 |
| ATOM | 981 | CA | ALA | A | 129 | 52.859 | −1.837 | 83.413 | 1.00 | 0.00 | xxxx | 981 |
| ATOM | 982 | C | ALA | A | 129 | 52.549 | −1.841 | 84.911 | 1.00 | 0.00 | xxxx | 982 |
| ATOM | 983 | O | ALA | A | 129 | 51.876 | −2.744 | 85.401 | 1.00 | 0.00 | xxxx | 983 |
| ATOM | 984 | CB | ALA | A | 129 | 53.793 | −2.995 | 83.084 | 1.00 | 0.00 | xxxx | 984 |
| ATOM | 985 | N | ASN | A | 130 | 53.053 | −0.847 | 85.636 | 1.00 | 0.00 | xxxx | 985 |
| ATOM | 986 | CA | ASN | A | 130 | 52.878 | −0.801 | 87.086 | 1.00 | 0.00 | xxxx | 986 |
| ATOM | 987 | C | ASN | A | 130 | 52.454 | 0.593 | 87.527 | 1.00 | 0.00 | xxxx | 987 |
| ATOM | 988 | O | ASN | A | 130 | 53.226 | 1.290 | 88.173 | 1.00 | 0.00 | xxxx | 988 |
| ATOM | 989 | CB | ASN | A | 130 | 54.179 | −1.189 | 87.808 | 1.00 | 0.00 | xxxx | 989 |
| ATOM | 990 | CG | ASN | A | 130 | 54.592 | −2.629 | 87.551 | 1.00 | 0.00 | xxxx | 990 |
| ATOM | 991 | OD1 | ASN | A | 130 | 54.189 | −3.538 | 88.283 | 1.00 | 0.00 | xxxx | 991 |
| ATOM | 992 | ND2 | ASN | A | 130 | 55.397 | −2.847 | 86.509 | 1.00 | 0.00 | xxxx | 992 |
| ATOM | 993 | N | GLN | A | 131 | 51.236 | 1.005 | 87.192 | 1.00 | 0.00 | xxxx | 993 |
| ATOM | 994 | CA | GLN | A | 131 | 50.836 | 2.377 | 87.493 | 1.00 | 0.00 | xxxx | 994 |
| ATOM | 995 | C | GLN | A | 131 | 50.664 | 2.616 | 88.994 | 1.00 | 0.00 | xxxx | 995 |
| ATOM | 996 | O | GLN | A | 131 | 50.591 | 3.756 | 89.434 | 1.00 | 0.00 | xxxx | 996 |
| ATOM | 997 | CB | GLN | A | 131 | 49.558 | 2.737 | 86.743 | 1.00 | 0.00 | xxxx | 997 |
| ATOM | 998 | CG | GLN | A | 131 | 49.811 | 3.113 | 85.287 | 1.00 | 0.00 | xxxx | 998 |
| ATOM | 999 | CD | GLN | A | 131 | 50.666 | 4.365 | 85.145 | 1.00 | 0.00 | xxxx | 999 |
| ATOM | 1000 | OE1 | GLN | A | 131 | 50.176 | 5.486 | 85.303 | 1.00 | 0.00 | xxxx | 1000 |
| ATOM | 1001 | NE2 | GLN | A | 131 | 51.949 | 4.180 | 84.848 | 1.00 | 0.00 | xxxx | 1001 |
| ATOM | 1002 | N | GLY | A | 132 | 50.627 | 1.541 | 89.773 | 1.00 | 0.00 | xxxx | 1002 |
| ATOM | 1003 | CA | GLY | A | 132 | 50.664 | 1.661 | 91.218 | 1.00 | 0.00 | xxxx | 1003 |
| ATOM | 1004 | C | GLY | A | 132 | 51.957 | 2.255 | 91.741 | 1.00 | 0.00 | xxxx | 1004 |
| ATOM | 1005 | O | GLY | A | 132 | 52.014 | 2.708 | 92.886 | 1.00 | 0.00 | xxxx | 1005 |
| ATOM | 1006 | N | TRP | A | 133 | 52.999 | 2.266 | 90.906 | 1.00 | 0.00 | xxxx | 1006 |
| ATOM | 1007 | CA | TRP | A | 133 | 54.265 | 2.885 | 91.281 | 1.00 | 0.00 | xxxx | 1007 |
| ATOM | 1008 | C | TRP | A | 133 | 54.179 | 4.410 | 91.375 | 1.00 | 0.00 | xxxx | 1008 |
| ATOM | 1009 | O | TRP | A | 133 | 55.021 | 5.042 | 92.004 | 1.00 | 0.00 | xxxx | 1009 |
| ATOM | 1010 | CB | TRP | A | 133 | 55.369 | 2.526 | 90.279 | 1.00 | 0.00 | xxxx | 1010 |
| ATOM | 1011 | CG | TRP | A | 133 | 55.859 | 1.107 | 90.312 | 1.00 | 0.00 | xxxx | 1011 |
| ATOM | 1012 | CD1 | TRP | A | 133 | 55.501 | 0.119 | 91.186 | 1.00 | 0.00 | xxxx | 1012 |
| ATOM | 1013 | CD2 | TRP | A | 133 | 56.818 | 0.531 | 89.423 | 1.00 | 0.00 | xxxx | 1013 |
| ATOM | 1014 | NE1 | TRP | A | 133 | 56.190 | −1.045 | 90.890 | 1.00 | 0.00 | xxxx | 1014 |
| ATOM | 1015 | CE2 | TRP | A | 133 | 56.997 | −0.816 | 89.806 | 1.00 | 0.00 | xxxx | 1015 |
| ATOM | 1016 | CE3 | TRP | A | 133 | 57.544 | 1.025 | 88.334 | 1.00 | 0.00 | xxxx | 1016 |
| ATOM | 1017 | CZ2 | TRP | A | 133 | 57.877 | −1.674 | 89.140 | 1.00 | 0.00 | xxxx | 1017 |
| ATOM | 1018 | CZ3 | TRP | A | 133 | 58.422 | 0.170 | 87.671 | 1.00 | 0.00 | xxxx | 1018 |
| ATOM | 1019 | CH2 | TRP | A | 133 | 58.574 | −1.165 | 88.079 | 1.00 | 0.00 | xxxx | 1019 |
| ATOM | 1020 | N | ASP | A | 134 | 53.175 | 4.985 | 90.714 | 1.00 | 0.00 | xxxx | 1020 |
| ATOM | 1021 | CA | ASP | A | 134 | 52.940 | 6.423 | 90.756 | 1.00 | 0.00 | xxxx | 1021 |
| ATOM | 1022 | C | ASP | A | 134 | 52.241 | 6.760 | 92.070 | 1.00 | 0.00 | xxxx | 1022 |
| ATOM | 1023 | O | ASP | A | 134 | 51.022 | 6.966 | 92.112 | 1.00 | 0.00 | xxxx | 1023 |
| ATOM | 1024 | CB | ASP | A | 134 | 52.105 | 6.842 | 89.543 | 1.00 | 0.00 | xxxx | 1024 |
| ATOM | 1025 | CG | ASP | A | 134 | 51.978 | 8.350 | 89.382 | 1.00 | 0.00 | xxxx | 1025 |
| ATOM | 1026 | OD1 | ASP | A | 134 | 52.701 | 9.111 | 90.044 | 1.00 | 0.00 | xxxx | 1026 |
| ATOM | 1027 | OD2 | ASP | A | 134 | 51.151 | 8.771 | 88.554 | 1.00 | 0.00 | xxxx | 1027 |
| ATOM | 1028 | N | LEU | A | 135 | 53.031 | 6.800 | 93.141 | 1.00 | 0.00 | xxxx | 1028 |
| ATOM | 1029 | CA | LEU | A | 135 | 52.494 | 6.901 | 94.496 | 1.00 | 0.00 | xxxx | 1029 |
| ATOM | 1030 | C | LEU | A | 135 | 51.661 | 8.154 | 94.723 | 1.00 | 0.00 | xxxx | 1030 |
| ATOM | 1031 | O | LEU | A | 135 | 50.639 | 8.093 | 95.418 | 1.00 | 0.00 | xxxx | 1031 |
| ATOM | 1032 | CB | LEU | A | 135 | 53.636 | 6.852 | 95.513 | 1.00 | 0.00 | xxxx | 1032 |
| ATOM | 1033 | CG | LEU | A | 135 | 54.522 | 5.609 | 95.486 | 1.00 | 0.00 | xxxx | 1033 |
| ATOM | 1034 | CD1 | LEU | A | 135 | 55.586 | 5.739 | 96.573 | 1.00 | 0.00 | xxxx | 1034 |
| ATOM | 1035 | CD2 | LEU | A | 135 | 53.695 | 4.365 | 95.696 | 1.00 | 0.00 | xxxx | 1035 |
| ATOM | 1036 | N | ASN | A | 136 | 52.068 | 9.286 | 94.148 | 1.00 | 0.00 | xxxx | 1036 |
| ATOM | 1037 | CA | ASN | A | 136 | 51.282 | 10.512 | 94.345 | 1.00 | 0.00 | xxxx | 1037 |
| ATOM | 1038 | C | ASN | A | 136 | 50.294 | 10.778 | 93.208 | 1.00 | 0.00 | xxxx | 1038 |
| ATOM | 1039 | O | ASN | A | 136 | 49.642 | 11.827 | 93.183 | 1.00 | 0.00 | xxxx | 1039 |
| ATOM | 1040 | CB | ASN | A | 136 | 52.193 | 11.729 | 94.543 | 1.00 | 0.00 | xxxx | 1040 |
| ATOM | 1041 | CG | ASN | A | 136 | 52.931 | 12.144 | 93.274 | 1.00 | 0.00 | xxxx | 1041 |
| ATOM | 1042 | OD1 | ASN | A | 136 | 53.028 | 11.386 | 92.307 | 1.00 | 0.00 | xxxx | 1042 |
| ATOM | 1043 | ND2 | ASN | A | 136 | 53.477 | 13.362 | 93.289 | 1.00 | 0.00 | xxxx | 1043 |
| ATOM | 1044 | N | LYS | A | 137 | 50.197 | 9.830 | 92.280 | 1.00 | 0.00 | xxxx | 1044 |
| ATOM | 1045 | CA | LYS | A | 137 | 49.160 | 9.856 | 91.239 | 1.00 | 0.00 | xxxx | 1045 |
| ATOM | 1046 | C | LYS | A | 137 | 49.225 | 11.109 | 90.363 | 1.00 | 0.00 | xxxx | 1046 |
| ATOM | 1047 | O | LYS | A | 137 | 48.189 | 11.578 | 89.883 | 1.00 | 0.00 | xxxx | 1047 |
| ATOM | 1048 | CB | LYS | A | 137 | 47.770 | 9.736 | 91.883 | 1.00 | 0.00 | xxxx | 1048 |
| ATOM | 1049 | CG | LYS | A | 137 | 47.571 | 8.425 | 92.630 | 1.00 | 0.00 | xxxx | 1049 |
| ATOM | 1050 | CD | LYS | A | 137 | 46.208 | 8.359 | 93.303 | 1.00 | 0.00 | xxxx | 1050 |
| ATOM | 1051 | CE | LYS | A | 137 | 45.091 | 8.308 | 92.284 | 1.00 | 0.00 | xxxx | 1051 |
| ATOM | 1052 | NZ | LYS | A | 137 | 45.115 | 7.052 | 91.487 | 1.00 | 0.00 | xxxx | 1052 |
| ATOM | 1053 | N | ASP | A | 138 | 50.428 | 11.638 | 90.135 | 1.00 | 0.00 | xxxx | 1053 |
| ATOM | 1054 | CA | ASP | A | 138 | 50.554 | 12.869 | 89.344 | 1.00 | 0.00 | xxxx | 1054 |
| ATOM | 1055 | C | ASP | A | 138 | 51.009 | 12.636 | 87.901 | 1.00 | 0.00 | xxxx | 1055 |
| ATOM | 1056 | O | ASP | A | 138 | 51.184 | 13.594 | 87.148 | 1.00 | 0.00 | xxxx | 1056 |
| ATOM | 1057 | CB | ASP | A | 138 | 51.499 | 13.865 | 90.041 | 1.00 | 0.00 | xxxx | 1057 |
| ATOM | 1058 | CG | ASP | A | 138 | 52.965 | 13.447 | 90.011 | 1.00 | 0.00 | xxxx | 1058 |
| ATOM | 1059 | OD1 | ASP | A | 138 | 53.285 | 12.308 | 89.603 | 1.00 | 0.00 | xxxx | 1059 |
| ATOM | 1060 | OD2 | ASP | A | 138 | 53.812 | 14.281 | 90.431 | 1.00 | 0.00 | xxxx | 1060 |
| ATOM | 1061 | N | GLY | A | 139 | 51.196 | 11.374 | 87.519 | 1.00 | 0.00 | xxxx | 1061 |
| ATOM | 1062 | CA | GLY | A | 139 | 51.634 | 11.030 | 86.174 | 1.00 | 0.00 | xxxx | 1062 |
| ATOM | 1063 | C | GLY | A | 139 | 53.104 | 11.282 | 85.887 | 1.00 | 0.00 | xxxx | 1063 |
| ATOM | 1064 | O | GLY | A | 139 | 53.555 | 11.086 | 84.757 | 1.00 | 0.00 | xxxx | 1064 |
| ATOM | 1065 | N | GLN | A | 140 | 53.859 | 11.686 | 86.910 | 1.00 | 0.00 | xxxx | 1065 |
| ATOM | 1066 | CA | GLN | A | 140 | 55.299 | 11.929 | 86.779 | 1.00 | 0.00 | xxxx | 1066 |
| ATOM | 1067 | C | GLN | A | 140 | 56.057 | 10.927 | 87.603 | 1.00 | 0.00 | xxxx | 1067 |
| ATOM | 1068 | O | GLN | A | 140 | 55.531 | 10.447 | 88.612 | 1.00 | 0.00 | xxxx | 1068 |
| ATOM | 1069 | CB | GLN | A | 140 | 55.682 | 13.327 | 87.258 | 1.00 | 0.00 | xxxx | 1069 |
| ATOM | 1070 | CG | GLN | A | 140 | 54.808 | 14.436 | 86.722 | 1.00 | 0.00 | xxxx | 1070 |
| ATOM | 1071 | CD | GLN | A | 140 | 55.319 | 15.804 | 87.115 | 1.00 | 0.00 | xxxx | 1071 |
| ATOM | 1072 | OE1 | GLN | A | 140 | 56.517 | 15.992 | 87.328 | 1.00 | 0.00 | xxxx | 1072 |
| ATOM | 1073 | NE2 | GLN | A | 140 | 54.412 | 16.769 | 87.222 | 1.00 | 0.00 | xxxx | 1073 |
| ATOM | 1074 | N | ILE | A | 141 | 57.275 | 10.594 | 87.178 | 1.00 | 0.00 | xxxx | 1074 |
| ATOM | 1075 | CA | ILE | A | 141 | 58.146 | 9.760 | 88.003 | 1.00 | 0.00 | xxxx | 1075 |
| ATOM | 1076 | C | ILE | A | 141 | 58.978 | 10.606 | 88.964 | 1.00 | 0.00 | xxxx | 1076 |
| ATOM | 1077 | O | ILE | A | 141 | 59.856 | 11.347 | 88.538 | 1.00 | 0.00 | xxxx | 1077 |
| ATOM | 1078 | CB | ILE | A | 141 | 59.090 | 8.899 | 87.162 | 1.00 | 0.00 | xxxx | 1078 |
| ATOM | 1079 | CG1 | ILE | A | 141 | 58.300 | 7.990 | 86.223 | 1.00 | 0.00 | xxxx | 1079 |
| ATOM | 1080 | CD1 | ILE | A | 141 | 59.209 | 7.121 | 85.330 | 1.00 | 0.00 | xxxx | 1080 |
| ATOM | 1081 | CG2 | ILE | A | 141 | 60.002 | 8.087 | 88.090 | 1.00 | 0.00 | xxxx | 1081 |
| ATOM | 1082 | N | GLN | A | 142 | 58.695 | 10.497 | 90.256 | 1.00 | 0.00 | xxxx | 1082 |
| ATOM | 1083 | CA | GLN | A | 142 | 59.568 | 11.073 | 91.274 | 1.00 | 0.00 | xxxx | 1083 |
| ATOM | 1084 | C | GLN | A | 142 | 60.693 | 10.084 | 91.528 | 1.00 | 0.00 | xxxx | 1084 |
| ATOM | 1085 | O | GLN | A | 142 | 60.453 | 8.970 | 92.000 | 1.00 | 0.00 | xxxx | 1085 |
| ATOM | 1086 | CB | GLN | A | 142 | 58.795 | 11.361 | 92.562 | 1.00 | 0.00 | xxxx | 1086 |
| ATOM | 1087 | CG | GLN | A | 142 | 58.111 | 12.718 | 92.607 | 1.00 | 0.00 | xxxx | 1087 |
| ATOM | 1088 | CD | GLN | A | 142 | 56.877 | 12.796 | 91.716 | 1.00 | 0.00 | xxxx | 1088 |
| ATOM | 1089 | OE1 | GLN | A | 142 | 56.161 | 11.805 | 91.524 | 1.00 | 0.00 | xxxx | 1089 |
| ATOM | 1090 | NE2 | GLN | A | 142 | 56.611 | 13.987 | 91.190 | 1.00 | 0.00 | xxxx | 1090 |
| ATOM | 1091 | N | PHE | A | 143 | 61.916 | 10.468 | 91.183 | 1.00 | 0.00 | xxxx | 1091 |
| ATOM | 1092 | CA | PHE | A | 143 | 63.031 | 9.530 | 91.267 | 1.00 | 0.00 | xxxx | 1092 |
| ATOM | 1093 | C | PHE | A | 143 | 64.180 | 10.041 | 92.119 | 1.00 | 0.00 | xxxx | 1093 |
| ATOM | 1094 | O | PHE | A | 143 | 64.290 | 11.237 | 92.391 | 1.00 | 0.00 | xxxx | 1094 |
| ATOM | 1095 | CB | PHE | A | 143 | 63.561 | 9.192 | 89.856 | 1.00 | 0.00 | xxxx | 1095 |
| ATOM | 1096 | CG | PHE | A | 143 | 64.318 | 10.321 | 89.187 | 1.00 | 0.00 | xxxx | 1096 |
| ATOM | 1097 | CD1 | PHE | A | 143 | 65.710 | 10.383 | 89.259 | 1.00 | 0.00 | xxxx | 1097 |
| ATOM | 1098 | CD2 | PHE | A | 143 | 63.647 | 11.301 | 88.475 | 1.00 | 0.00 | xxxx | 1098 |
| ATOM | 1099 | CE1 | PHE | A | 143 | 66.415 | 11.409 | 88.638 | 1.00 | 0.00 | xxxx | 1099 |
| ATOM | 1100 | CE2 | PHE | A | 143 | 64.342 | 12.332 | 87.852 | 1.00 | 0.00 | xxxx | 1100 |
| ATOM | 1101 | CZ | PHE | A | 143 | 65.730 | 12.386 | 87.936 | 1.00 | 0.00 | xxxx | 1101 |
| ATOM | 1102 | N | VAL | A | 144 | 65.038 | 9.116 | 92.534 | 1.00 | 0.00 | xxxx | 1102 |
| ATOM | 1103 | CA | VAL | A | 144 | 66.359 | 9.480 | 93.032 | 1.00 | 0.00 | xxxx | 1103 |
| ATOM | 1104 | C | VAL | A | 144 | 67.364 | 8.774 | 92.143 | 1.00 | 0.00 | xxxx | 1104 |
| ATOM | 1105 | O | VAL | A | 144 | 67.052 | 7.737 | 91.557 | 1.00 | 0.00 | xxxx | 1105 |
| ATOM | 1106 | CB | VAL | A | 144 | 66.579 | 9.111 | 94.511 | 1.00 | 0.00 | xxxx | 1106 |
| ATOM | 1107 | CG1 | VAL | A | 144 | 65.579 | 9.852 | 95.400 | 1.00 | 0.00 | xxxx | 1107 |
| ATOM | 1108 | CG2 | VAL | A | 144 | 66.496 | 7.598 | 94.726 | 1.00 | 0.00 | xxxx | 1108 |
| ATOM | 1109 | N | LEU | A | 145 | 68.556 | 9.348 | 92.009 | 1.00 | 0.00 | xxxx | 1109 |
| ATOM | 1110 | CA | LEU | A | 145 | 69.551 | 8.754 | 91.118 | 1.00 | 0.00 | xxxx | 1110 |
| ATOM | 1111 | C | LEU | A | 145 | 70.913 | 8.649 | 91.792 | 1.00 | 0.00 | xxxx | 1111 |
| ATOM | 1112 | O | LEU | A | 145 | 71.429 | 9.620 | 92.348 | 1.00 | 0.00 | xxxx | 1112 |
| ATOM | 1113 | CB | LEU | A | 145 | 69.651 | 9.557 | 89.808 | 1.00 | 0.00 | xxxx | 1113 |
| ATOM | 1114 | CG | LEU | A | 145 | 70.503 | 8.946 | 88.676 | 1.00 | 0.00 | xxxx | 1114 |
| ATOM | 1115 | CD1 | LEU | A | 145 | 69.943 | 9.329 | 87.311 | 1.00 | 0.00 | xxxx | 1115 |
| ATOM | 1116 | CD2 | LEU | A | 145 | 71.965 | 9.353 | 88.771 | 1.00 | 0.00 | xxxx | 1116 |
| ATOM | 1117 | N | LEU | A | 146 | 71.462 | 7.438 | 91.735 | 1.00 | 0.00 | xxxx | 1117 |
| ATOM | 1118 | CA | LEU | A | 146 | 72.791 | 7.139 | 92.259 | 1.00 | 0.00 | xxxx | 1118 |
| ATOM | 1119 | C | LEU | A | 146 | 73.792 | 7.143 | 91.107 | 1.00 | 0.00 | xxxx | 1119 |
| ATOM | 1120 | O | LEU | A | 146 | 73.759 | 6.267 | 90.238 | 1.00 | 0.00 | xxxx | 1120 |
| ATOM | 1121 | CB | LEU | A | 146 | 72.770 | 5.788 | 92.984 | 1.00 | 0.00 | xxxx | 1121 |
| ATOM | 1122 | CG | LEU | A | 146 | 71.969 | 5.854 | 94.285 | 1.00 | 0.00 | xxxx | 1122 |
| ATOM | 1123 | CD1 | LEU | A | 146 | 71.464 | 4.479 | 94.688 | 1.00 | 0.00 | xxxx | 1123 |
| ATOM | 1124 | CD2 | LEU | A | 146 | 72.845 | 6.450 | 95.380 | 1.00 | 0.00 | xxxx | 1124 |
| ATOM | 1125 | N | LYS | A | 147 | 74.662 | 8.147 | 91.095 | 1.00 | 0.00 | xxxx | 1125 |
| ATOM | 1126 | CA | LYS | A | 147 | 75.583 | 8.361 | 89.986 | 1.00 | 0.00 | xxxx | 1126 |
| ATOM | 1127 | C | LYS | A | 147 | 76.939 | 7.700 | 90.244 | 1.00 | 0.00 | xxxx | 1127 |
| ATOM | 1128 | O | LYS | A | 147 | 77.365 | 7.565 | 91.391 | 1.00 | 0.00 | xxxx | 1128 |
| ATOM | 1129 | CB | LYS | A | 147 | 75.739 | 9.871 | 89.754 | 1.00 | 0.00 | xxxx | 1129 |
| ATOM | 1130 | CG | LYS | A | 147 | 76.634 | 10.249 | 88.595 | 1.00 | 0.00 | xxxx | 1130 |
| ATOM | 1131 | CD | LYS | A | 147 | 76.509 | 11.741 | 88.309 | 1.00 | 0.00 | xxxx | 1131 |
| ATOM | 1132 | CE | LYS | A | 147 | 77.457 | 12.148 | 87.189 | 1.00 | 0.00 | xxxx | 1132 |
| ATOM | 1133 | NZ | LYS | A | 147 | 77.409 | 13.623 | 86.951 | 1.00 | 0.00 | xxxx | 1133 |
| ATOM | 1134 | N | GLY | A | 148 | 77.607 | 7.268 | 89.174 | 1.00 | 0.00 | xxxx | 1134 |
| ATOM | 1135 | CA | GLY | A | 148 | 78.973 | 6.778 | 89.262 | 1.00 | 0.00 | xxxx | 1135 |
| ATOM | 1136 | C | GLY | A | 148 | 80.004 | 7.876 | 89.465 | 1.00 | 0.00 | xxxx | 1136 |
| ATOM | 1137 | O | GLY | A | 148 | 79.660 | 8.999 | 89.844 | 1.00 | 0.00 | xxxx | 1137 |
| ATOM | 1138 | N | GLU | A | 149 | 81.269 | 7.544 | 89.201 | 1.00 | 0.00 | xxxx | 1138 |
| ATOM | 1139 | CA | GLU | A | 149 | 82.387 | 8.479 | 89.315 | 1.00 | 0.00 | xxxx | 1139 |
| ATOM | 1140 | C | GLU | A | 149 | 82.189 | 9.673 | 88.390 | 1.00 | 0.00 | xxxx | 1140 |
| ATOM | 1141 | O | GLU | A | 149 | 82.058 | 9.502 | 87.185 | 1.00 | 0.00 | xxxx | 1141 |
| ATOM | 1142 | CB | GLU | A | 149 | 83.705 | 7.763 | 88.982 | 1.00 | 0.00 | xxxx | 1142 |
| ATOM | 1143 | CG | GLU | A | 149 | 84.892 | 8.693 | 88.733 | 1.00 | 0.00 | xxxx | 1143 |
| ATOM | 1144 | CD | GLU | A | 149 | 86.163 | 7.955 | 88.312 | 1.00 | 0.00 | xxxx | 1144 |
| ATOM | 1145 | OE1 | GLU | A | 149 | 86.160 | 7.257 | 87.269 | 1.00 | 0.00 | xxxx | 1145 |
| ATOM | 1146 | OE2 | GLU | A | 149 | 87.176 | 8.078 | 89.032 | 1.00 | 0.00 | xxxx | 1146 |
| ATOM | 1147 | N | PRO | A | 150 | 82.139 | 10.889 | 88.954 | 1.00 | 0.00 | xxxx | 1147 |
| ATOM | 1148 | CA | PRO | A | 150 | 81.976 | 12.064 | 88.090 | 1.00 | 0.00 | xxxx | 1148 |
| ATOM | 1149 | C | PRO | A | 150 | 83.076 | 12.150 | 87.034 | 1.00 | 0.00 | xxxx | 1149 |
| ATOM | 1150 | O | PRO | A | 150 | 84.254 | 11.966 | 87.346 | 1.00 | 0.00 | xxxx | 1150 |
| ATOM | 1151 | CB | PRO | A | 150 | 82.054 | 13.233 | 89.076 | 1.00 | 0.00 | xxxx | 1151 |
| ATOM | 1152 | CG | PRO | A | 150 | 81.585 | 12.636 | 90.382 | 1.00 | 0.00 | xxxx | 1152 |
| ATOM | 1153 | CD | PRO | A | 150 | 82.150 | 11.246 | 90.383 | 1.00 | 0.00 | xxxx | 1153 |
| ATOM | 1154 | N | GLY | A | 151 | 82.686 | 12.388 | 85.786 | 1.00 | 0.00 | xxxx | 1154 |
| ATOM | 1155 | CA | GLY | A | 151 | 83.636 | 12.431 | 84.690 | 1.00 | 0.00 | xxxx | 1155 |
| ATOM | 1156 | C | GLY | A | 151 | 83.867 | 11.116 | 83.969 | 1.00 | 0.00 | xxxx | 1156 |
| ATOM | 1157 | O | GLY | A | 151 | 84.386 | 11.100 | 82.852 | 1.00 | 0.00 | xxxx | 1157 |
| ATOM | 1158 | N | HIS | A | 152 | 83.505 | 10.008 | 84.610 | 1.00 | 0.00 | xxxx | 1158 |
| ATOM | 1159 | CA | HIS | A | 152 | 83.556 | 8.702 | 83.962 | 1.00 | 0.00 | xxxx | 1159 |
| ATOM | 1160 | C | HIS | A | 152 | 82.542 | 8.753 | 82.832 | 1.00 | 0.00 | xxxx | 1160 |
| ATOM | 1161 | O | HIS | A | 152 | 81.397 | 9.117 | 83.066 | 1.00 | 0.00 | xxxx | 1161 |
| ATOM | 1162 | CB | HIS | A | 152 | 83.232 | 7.586 | 84.968 | 1.00 | 0.00 | xxxx | 1162 |
| ATOM | 1163 | CG | HIS | A | 152 | 83.493 | 6.196 | 84.467 | 1.00 | 0.00 | xxxx | 1163 |
| ATOM | 1164 | ND1 | HIS | A | 152 | 82.885 | 5.674 | 83.345 | 1.00 | 0.00 | xxxx | 1164 |
| ATOM | 1165 | CD2 | HIS | A | 152 | 84.270 | 5.205 | 84.966 | 1.00 | 0.00 | xxxx | 1165 |
| ATOM | 1166 | CE1 | HIS | A | 152 | 83.290 | 4.429 | 83.162 | 1.00 | 0.00 | xxxx | 1166 |
| ATOM | 1167 | NE2 | HIS | A | 152 | 84.131 | 4.120 | 84.134 | 1.00 | 0.00 | xxxx | 1167 |
| ATOM | 1168 | N | PRO | A | 153 | 82.963 | 8.418 | 81.602 | 1.00 | 0.00 | xxxx | 1168 |
| ATOM | 1169 | CA | PRO | A | 153 | 82.057 | 8.494 | 80.450 | 1.00 | 0.00 | xxxx | 1169 |
| ATOM | 1170 | C | PRO | A | 153 | 80.746 | 7.753 | 80.671 | 1.00 | 0.00 | xxxx | 1170 |
| ATOM | 1171 | O | PRO | A | 153 | 79.684 | 8.243 | 80.282 | 1.00 | 0.00 | xxxx | 1171 |
| ATOM | 1172 | CB | PRO | A | 153 | 82.867 | 7.831 | 79.330 | 1.00 | 0.00 | xxxx | 1172 |
| ATOM | 1173 | CG | PRO | A | 153 | 84.262 | 8.052 | 79.713 | 1.00 | 0.00 | xxxx | 1173 |
| ATOM | 1174 | CD | PRO | A | 153 | 84.313 | 7.980 | 81.209 | 1.00 | 0.00 | xxxx | 1174 |
| ATOM | 1175 | N | ASP | A | 154 | 80.818 | 6.585 | 81.300 | 1.00 | 0.00 | xxxx | 1175 |
| ATOM | 1176 | CA | ASP | A | 154 | 79.619 | 5.782 | 81.532 | 1.00 | 0.00 | xxxx | 1176 |
| ATOM | 1177 | C | ASP | A | 154 | 78.681 | 6.459 | 82.518 | 1.00 | 0.00 | xxxx | 1177 |
| ATOM | 1178 | O | ASP | A | 154 | 77.477 | 6.478 | 82.308 | 1.00 | 0.00 | xxxx | 1178 |
| ATOM | 1179 | CB | ASP | A | 154 | 79.969 | 4.382 | 82.041 | 1.00 | 0.00 | xxxx | 1179 |
| ATOM | 1180 | CG | ASP | A | 154 | 80.549 | 3.488 | 80.956 | 1.00 | 0.00 | xxxx | 1180 |
| ATOM | 1181 | OD1 | ASP | A | 154 | 80.282 | 3.727 | 79.756 | 1.00 | 0.00 | xxxx | 1181 |
| ATOM | 1182 | OD2 | ASP | A | 154 | 81.262 | 2.524 | 81.298 | 1.00 | 0.00 | xxxx | 1182 |
| ATOM | 1183 | N | ALA | A | 155 | 79.225 | 6.992 | 83.609 | 1.00 | 0.00 | xxxx | 1183 |
| ATOM | 1184 | CA | ALA | A | 155 | 78.391 | 7.677 | 84.597 | 1.00 | 0.00 | xxxx | 1184 |
| ATOM | 1185 | C | ALA | A | 155 | 77.702 | 8.915 | 84.010 | 1.00 | 0.00 | xxxx | 1185 |
| ATOM | 1186 | O | ALA | A | 155 | 76.509 | 9.131 | 84.215 | 1.00 | 0.00 | xxxx | 1186 |
| ATOM | 1187 | CB | ALA | A | 155 | 79.220 | 8.067 | 85.819 | 1.00 | 0.00 | xxxx | 1187 |
| ATOM | 1188 | N | GLU | A | 156 | 78.455 | 9.731 | 83.276 | 1.00 | 0.00 | xxxx | 1188 |
| ATOM | 1189 | CA | GLU | A | 156 | 77.885 | 10.946 | 82.720 | 1.00 | 0.00 | xxxx | 1189 |
| ATOM | 1190 | C | GLU | A | 156 | 76.799 | 10.637 | 81.693 | 1.00 | 0.00 | xxxx | 1190 |
| ATOM | 1191 | O | GLU | A | 156 | 75.725 | 11.221 | 81.738 | 1.00 | 0.00 | xxxx | 1191 |
| ATOM | 1192 | CB | GLU | A | 156 | 78.973 | 11.814 | 82.091 | 1.00 | 0.00 | xxxx | 1192 |
| ATOM | 1193 | CG | GLU | A | 156 | 80.011 | 12.322 | 83.083 | 1.00 | 0.00 | xxxx | 1193 |
| ATOM | 1194 | CD | GLU | A | 156 | 79.422 | 13.168 | 84.199 | 1.00 | 0.00 | xxxx | 1194 |
| ATOM | 1195 | OE1 | GLU | A | 156 | 78.347 | 13.780 | 84.009 | 1.00 | 0.00 | xxxx | 1195 |
| ATOM | 1196 | OE2 | GLU | A | 156 | 80.049 | 13.224 | 85.276 | 1.00 | 0.00 | xxxx | 1196 |
| ATOM | 1197 | N | ALA | A | 157 | 77.090 | 9.729 | 80.765 | 1.00 | 0.00 | xxxx | 1197 |
| ATOM | 1198 | CA | ALA | A | 157 | 76.121 | 9.347 | 79.745 | 1.00 | 0.00 | xxxx | 1198 |
| ATOM | 1199 | C | ALA | A | 157 | 74.890 | 8.658 | 80.332 | 1.00 | 0.00 | xxxx | 1199 |
| ATOM | 1200 | O | ALA | A | 157 | 73.761 | 8.943 | 79.922 | 1.00 | 0.00 | xxxx | 1200 |
| ATOM | 1201 | CB | ALA | A | 157 | 76.775 | 8.439 | 78.713 | 1.00 | 0.00 | xxxx | 1201 |
| ATOM | 1202 | N | ARG | A | 158 | 75.085 | 7.754 | 81.286 | 1.00 | 0.00 | xxxx | 1202 |
| ATOM | 1203 | CA | ARG | A | 158 | 73.946 | 7.028 | 81.836 | 1.00 | 0.00 | xxxx | 1203 |
| ATOM | 1204 | C | ARG | A | 158 | 73.015 | 7.947 | 82.600 | 1.00 | 0.00 | xxxx | 1204 |
| ATOM | 1205 | O | ARG | A | 158 | 71.795 | 7.761 | 82.596 | 1.00 | 0.00 | xxxx | 1205 |
| ATOM | 1206 | CB | ARG | A | 158 | 74.413 | 5.885 | 82.742 | 1.00 | 0.00 | xxxx | 1206 |
| ATOM | 1207 | CG | ARG | A | 158 | 74.963 | 4.718 | 81.960 | 1.00 | 0.00 | xxxx | 1207 |
| ATOM | 1208 | CD | ARG | A | 158 | 75.686 | 3.742 | 82.866 | 1.00 | 0.00 | xxxx | 1208 |
| ATOM | 1209 | NE | ARG | A | 158 | 76.454 | 2.793 | 82.073 | 1.00 | 0.00 | xxxx | 1209 |
| ATOM | 1210 | CZ | ARG | A | 158 | 77.307 | 1.917 | 82.597 | 1.00 | 0.00 | xxxx | 1210 |
| ATOM | 1211 | NH1 | ARG | A | 158 | 77.475 | 1.869 | 83.918 | 1.00 | 0.00 | xxxx | 1211 |
| ATOM | 1212 | NH2 | ARG | A | 158 | 78.000 | 1.104 | 81.808 | 1.00 | 0.00 | xxxx | 1212 |
| ATOM | 1213 | N | THR | A | 159 | 73.595 | 8.932 | 83.272 | 1.00 | 0.00 | xxxx | 1213 |
| ATOM | 1214 | CA | THR | A | 159 | 72.812 | 9.858 | 84.072 | 1.00 | 0.00 | xxxx | 1214 |
| ATOM | 1215 | C | THR | A | 159 | 72.016 | 10.787 | 83.165 | 1.00 | 0.00 | xxxx | 1215 |
| ATOM | 1216 | O | THR | A | 159 | 70.829 | 11.022 | 83.385 | 1.00 | 0.00 | xxxx | 1216 |
| ATOM | 1217 | CB | THR | A | 159 | 73.723 | 10.662 | 85.000 | 1.00 | 0.00 | xxxx | 1217 |
| ATOM | 1218 | OG1 | THR | A | 159 | 74.339 | 9.767 | 85.931 | 1.00 | 0.00 | xxxx | 1218 |
| ATOM | 1219 | CG2 | THR | A | 159 | 72.930 | 11.692 | 85.772 | 1.00 | 0.00 | xxxx | 1219 |
| ATOM | 1220 | N | THR | A | 160 | 72.673 | 11.288 | 82.128 | 1.00 | 0.00 | xxxx | 1220 |
| ATOM | 1221 | CA | THR | A | 160 | 72.007 | 12.189 | 81.196 | 1.00 | 0.00 | xxxx | 1221 |
| ATOM | 1222 | C | THR | A | 160 | 70.915 | 11.488 | 80.402 | 1.00 | 0.00 | xxxx | 1222 |
| ATOM | 1223 | O | THR | A | 160 | 69.805 | 12.009 | 80.263 | 1.00 | 0.00 | xxxx | 1223 |
| ATOM | 1224 | CB | THR | A | 160 | 73.011 | 12.800 | 80.207 | 1.00 | 0.00 | xxxx | 1224 |
| ATOM | 1225 | OG1 | THR | A | 160 | 73.939 | 13.636 | 80.914 | 1.00 | 0.00 | xxxx | 1225 |
| ATOM | 1226 | CG2 | THR | A | 160 | 72.289 | 13.625 | 79.137 | 1.00 | 0.00 | xxxx | 1226 |
| ATOM | 1227 | N | TYR | A | 161 | 71.237 | 10.319 | 79.853 | 1.00 | 0.00 | xxxx | 1227 |
| ATOM | 1228 | CA | TYR | A | 161 | 70.351 | 9.740 | 78.847 | 1.00 | 0.00 | xxxx | 1228 |
| ATOM | 1229 | C | TYR | A | 161 | 69.210 | 8.901 | 79.417 | 1.00 | 0.00 | xxxx | 1229 |
| ATOM | 1230 | O | TYR | A | 161 | 68.202 | 8.711 | 78.729 | 1.00 | 0.00 | xxxx | 1230 |
| ATOM | 1231 | CB | TYR | A | 161 | 71.169 | 8.925 | 77.829 | 1.00 | 0.00 | xxxx | 1231 |
| ATOM | 1232 | CG | TYR | A | 161 | 71.871 | 9.815 | 76.830 | 1.00 | 0.00 | xxxx | 1232 |
| ATOM | 1233 | CD1 | TYR | A | 161 | 71.230 | 10.222 | 75.669 | 1.00 | 0.00 | xxxx | 1233 |
| ATOM | 1234 | CD2 | TYR | A | 161 | 73.154 | 10.285 | 77.067 | 1.00 | 0.00 | xxxx | 1234 |
| ATOM | 1235 | CE1 | TYR | A | 161 | 71.864 | 11.051 | 74.756 | 1.00 | 0.00 | xxxx | 1235 |
| ATOM | 1236 | CE2 | TYR | A | 161 | 73.792 | 11.120 | 76.162 | 1.00 | 0.00 | xxxx | 1236 |
| ATOM | 1237 | CZ | TYR | A | 161 | 73.142 | 11.496 | 75.012 | 1.00 | 0.00 | xxxx | 1237 |
| ATOM | 1238 | OH | TYR | A | 161 | 73.775 | 12.323 | 74.112 | 1.00 | 0.00 | xxxx | 1238 |
| ATOM | 1239 | N | VAL | A | 162 | 69.310 | 8.434 | 80.659 | 1.00 | 0.00 | xxxx | 1239 |
| ATOM | 1240 | CA | VAL | A | 162 | 68.155 | 7.734 | 81.220 | 1.00 | 0.00 | xxxx | 1240 |
| ATOM | 1241 | C | VAL | A | 162 | 66.970 | 8.708 | 81.360 | 1.00 | 0.00 | xxxx | 1241 |
| ATOM | 1242 | O | VAL | A | 162 | 65.832 | 8.370 | 81.008 | 1.00 | 0.00 | xxxx | 1242 |
| ATOM | 1243 | CB | VAL | A | 162 | 68.495 | 7.027 | 82.564 | 1.00 | 0.00 | xxxx | 1243 |
| ATOM | 1244 | CG1 | VAL | A | 162 | 68.829 | 8.019 | 83.681 | 1.00 | 0.00 | xxxx | 1244 |
| ATOM | 1245 | CG2 | VAL | A | 162 | 67.339 | 6.120 | 82.992 | 1.00 | 0.00 | xxxx | 1245 |
| ATOM | 1246 | N | ILE | A | 163 | 67.249 | 9.926 | 81.817 | 1.00 | 0.00 | xxxx | 1246 |
| ATOM | 1247 | CA | ILE | A | 163 | 66.213 | 10.941 | 81.981 | 1.00 | 0.00 | xxxx | 1247 |
| ATOM | 1248 | C | ILE | A | 163 | 65.767 | 11.478 | 80.631 | 1.00 | 0.00 | xxxx | 1248 |
| ATOM | 1249 | O | ILE | A | 163 | 64.561 | 11.682 | 80.412 | 1.00 | 0.00 | xxxx | 1249 |
| ATOM | 1250 | CB | ILE | A | 163 | 66.702 | 12.073 | 82.917 | 1.00 | 0.00 | xxxx | 1250 |
| ATOM | 1251 | CG1 | ILE | A | 163 | 67.019 | 11.507 | 84.306 | 1.00 | 0.00 | xxxx | 1251 |
| ATOM | 1252 | CG2 | ILE | A | 163 | 65.663 | 13.205 | 83.013 | 1.00 | 0.00 | xxxx | 1252 |
| ATOM | 1253 | CD1 | ILE | A | 163 | 65.913 | 10.626 | 84.897 | 1.00 | 0.00 | xxxx | 1253 |
| ATOM | 1254 | N | LYS | A | 164 | 66.717 | 11.716 | 79.730 | 1.00 | 0.00 | xxxx | 1254 |
| ATOM | 1255 | CA | LYS | A | 164 | 66.365 | 12.228 | 78.410 | 1.00 | 0.00 | xxxx | 1255 |
| ATOM | 1256 | C | LYS | A | 164 | 65.417 | 11.261 | 77.700 | 1.00 | 0.00 | xxxx | 1256 |
| ATOM | 1257 | O | LYS | A | 164 | 64.434 | 11.676 | 77.079 | 1.00 | 0.00 | xxxx | 1257 |
| ATOM | 1258 | CB | LYS | A | 164 | 67.619 | 12.461 | 77.565 | 1.00 | 0.00 | xxxx | 1258 |
| ATOM | 1259 | CG | LYS | A | 164 | 67.313 | 13.048 | 76.199 | 1.00 | 0.00 | xxxx | 1259 |
| ATOM | 1260 | CD | LYS | A | 164 | 68.579 | 13.299 | 75.394 | 1.00 | 0.00 | xxxx | 1260 |
| ATOM | 1261 | CE | LYS | A | 164 | 68.249 | 13.646 | 73.948 | 1.00 | 0.00 | xxxx | 1261 |
| ATOM | 1262 | NZ | LYS | A | 164 | 67.187 | 14.687 | 73.850 | 1.00 | 0.00 | xxxx | 1262 |
| ATOM | 1263 | N | GLU | A | 165 | 65.703 | 9.969 | 77.807 | 1.00 | 0.00 | xxxx | 1263 |
| ATOM | 1264 | CA | GLU | A | 165 | 64.888 | 8.978 | 77.119 | 1.00 | 0.00 | xxxx | 1264 |
| ATOM | 1265 | C | GLU | A | 165 | 63.478 | 8.925 | 77.699 | 1.00 | 0.00 | xxxx | 1265 |
| ATOM | 1266 | O | GLU | A | 165 | 62.496 | 8.895 | 76.948 | 1.00 | 0.00 | xxxx | 1266 |
| ATOM | 1267 | CB | GLU | A | 165 | 65.548 | 7.595 | 77.174 | 1.00 | 0.00 | xxxx | 1267 |
| ATOM | 1268 | CG | GLU | A | 165 | 64.772 | 6.517 | 76.425 | 1.00 | 0.00 | xxxx | 1268 |
| ATOM | 1269 | CD | GLU | A | 165 | 64.739 | 6.725 | 74.909 | 1.00 | 0.00 | xxxx | 1269 |
| ATOM | 1270 | OE1 | GLU | A | 165 | 65.462 | 7.595 | 74.383 | 1.00 | 0.00 | xxxx | 1270 |
| ATOM | 1271 | OE2 | GLU | A | 165 | 63.978 | 6.001 | 74.234 | 1.00 | 0.00 | xxxx | 1271 |
| ATOM | 1272 | N | LEU | A | 166 | 63.367 | 8.943 | 79.024 | 1.00 | 0.00 | xxxx | 1272 |
| ATOM | 1273 | CA | LEU | A | 166 | 62.055 | 8.996 | 79.661 | 1.00 | 0.00 | xxxx | 1273 |
| ATOM | 1274 | C | LEU | A | 166 | 61.286 | 10.233 | 79.214 | 1.00 | 0.00 | xxxx | 1274 |
| ATOM | 1275 | O | LEU | A | 166 | 60.137 | 10.134 | 78.784 | 1.00 | 0.00 | xxxx | 1275 |
| ATOM | 1276 | CB | LEU | A | 166 | 62.196 | 8.980 | 81.185 | 1.00 | 0.00 | xxxx | 1276 |
| ATOM | 1277 | CG | LEU | A | 166 | 62.545 | 7.614 | 81.779 | 1.00 | 0.00 | xxxx | 1277 |
| ATOM | 1278 | CD1 | LEU | A | 166 | 63.027 | 7.747 | 83.220 | 1.00 | 0.00 | xxxx | 1278 |
| ATOM | 1279 | CD2 | LEU | A | 166 | 61.331 | 6.689 | 81.686 | 1.00 | 0.00 | xxxx | 1279 |
| ATOM | 1280 | N | ASN | A | 167 | 61.932 | 11.393 | 79.281 | 1.00 | 0.00 | xxxx | 1280 |
| ATOM | 1281 | CA | ASN | A | 167 | 61.263 | 12.636 | 78.902 | 1.00 | 0.00 | xxxx | 1281 |
| ATOM | 1282 | C | ASN | A | 167 | 60.872 | 12.639 | 77.421 | 1.00 | 0.00 | xxxx | 1282 |
| ATOM | 1283 | O | ASN | A | 167 | 59.788 | 13.121 | 77.071 | 1.00 | 0.00 | xxxx | 1283 |
| ATOM | 1284 | CB | ASN | A | 167 | 62.147 | 13.848 | 79.210 | 1.00 | 0.00 | xxxx | 1284 |
| ATOM | 1285 | CG | ASN | A | 167 | 62.376 | 14.059 | 80.705 | 1.00 | 0.00 | xxxx | 1285 |
| ATOM | 1286 | OD1 | ASN | A | 167 | 61.698 | 13.473 | 81.558 | 1.00 | 0.00 | xxxx | 1286 |
| ATOM | 1287 | ND2 | ASN | A | 167 | 63.329 | 14.915 | 81.024 | 1.00 | 0.00 | xxxx | 1287 |
| ATOM | 1288 | N | ASP | A | 168 | 61.730 | 12.088 | 76.558 | 1.00 | 0.00 | xxxx | 1288 |
| ATOM | 1289 | CA | ASP | A | 168 | 61.453 | 12.062 | 75.119 | 1.00 | 0.00 | xxxx | 1289 |
| ATOM | 1290 | C | ASP | A | 168 | 60.230 | 11.206 | 74.833 | 1.00 | 0.00 | xxxx | 1290 |
| ATOM | 1291 | O | ASP | A | 168 | 59.505 | 11.452 | 73.863 | 1.00 | 0.00 | xxxx | 1291 |
| ATOM | 1292 | CB | ASP | A | 168 | 62.658 | 11.534 | 74.332 | 1.00 | 0.00 | xxxx | 1292 |
| ATOM | 1293 | CG | ASP | A | 168 | 63.759 | 12.576 | 74.153 | 1.00 | 0.00 | xxxx | 1293 |
| ATOM | 1294 | OD1 | ASP | A | 168 | 63.522 | 13.772 | 74.436 | 1.00 | 0.00 | xxxx | 1294 |
| ATOM | 1295 | OD2 | ASP | A | 168 | 64.861 | 12.194 | 73.707 | 1.00 | 0.00 | xxxx | 1295 |
| ATOM | 1296 | N | LYS | A | 169 | 60.003 | 10.207 | 75.685 | 1.00 | 0.00 | xxxx | 1296 |
| ATOM | 1297 | CA | LYS | A | 169 | 58.855 | 9.311 | 75.552 | 1.00 | 0.00 | xxxx | 1297 |
| ATOM | 1298 | C | LYS | A | 169 | 57.585 | 9.897 | 76.161 | 1.00 | 0.00 | xxxx | 1298 |
| ATOM | 1299 | O | LYS | A | 169 | 56.543 | 9.243 | 76.175 | 1.00 | 0.00 | xxxx | 1299 |
| ATOM | 1300 | CB | LYS | A | 169 | 59.153 | 7.958 | 76.203 | 1.00 | 0.00 | xxxx | 1300 |
| ATOM | 1301 | CG | LYS | A | 169 | 60.182 | 7.127 | 75.469 | 1.00 | 0.00 | xxxx | 1301 |
| ATOM | 1302 | CD | LYS | A | 169 | 60.315 | 5.756 | 76.107 | 1.00 | 0.00 | xxxx | 1302 |
| ATOM | 1303 | CE | LYS | A | 169 | 60.991 | 4.783 | 75.159 | 1.00 | 0.00 | xxxx | 1303 |
| ATOM | 1304 | NZ | LYS | A | 169 | 60.305 | 4.701 | 73.837 | 1.00 | 0.00 | xxxx | 1304 |
| ATOM | 1305 | N | GLY | A | 170 | 57.667 | 11.127 | 76.657 | 1.00 | 0.00 | xxxx | 1305 |
| ATOM | 1306 | CA | GLY | A | 170 | 56.509 | 11.790 | 77.233 | 1.00 | 0.00 | xxxx | 1306 |
| ATOM | 1307 | C | GLY | A | 170 | 56.263 | 11.489 | 78.698 | 1.00 | 0.00 | xxxx | 1307 |
| ATOM | 1308 | O | GLY | A | 170 | 55.179 | 11.753 | 79.220 | 1.00 | 0.00 | xxxx | 1308 |
| ATOM | 1309 | N | ILE | A | 171 | 57.272 | 10.945 | 79.370 | 1.00 | 0.00 | xxxx | 1309 |
| ATOM | 1310 | CA | ILE | A | 171 | 57.172 | 10.698 | 80.804 | 1.00 | 0.00 | xxxx | 1310 |
| ATOM | 1311 | C | ILE | A | 171 | 57.941 | 11.780 | 81.540 | 1.00 | 0.00 | xxxx | 1311 |
| ATOM | 1312 | O | ILE | A | 171 | 59.177 | 11.813 | 81.507 | 1.00 | 0.00 | xxxx | 1312 |
| ATOM | 1313 | CB | ILE | A | 171 | 57.683 | 9.305 | 81.178 | 1.00 | 0.00 | xxxx | 1313 |
| ATOM | 1314 | CG1 | ILE | A | 171 | 56.891 | 8.229 | 80.423 | 1.00 | 0.00 | xxxx | 1314 |
| ATOM | 1315 | CD1 | ILE | A | 171 | 57.515 | 6.856 | 80.496 | 1.00 | 0.00 | xxxx | 1315 |
| ATOM | 1316 | CG2 | ILE | A | 171 | 57.608 | 9.094 | 82.690 | 1.00 | 0.00 | xxxx | 1316 |
| ATOM | 1317 | N | LYS | A | 172 | 57.208 | 12.683 | 82.187 | 1.00 | 0.00 | xxxx | 1317 |
| ATOM | 1318 | CA | LYS | A | 172 | 57.825 | 13.718 | 83.004 | 1.00 | 0.00 | xxxx | 1318 |
| ATOM | 1319 | C | LYS | A | 172 | 58.452 | 13.117 | 84.241 | 1.00 | 0.00 | xxxx | 1319 |
| ATOM | 1320 | O | LYS | A | 172 | 57.946 | 12.143 | 84.804 | 1.00 | 0.00 | xxxx | 1320 |
| ATOM | 1321 | CB | LYS | A | 172 | 56.805 | 14.770 | 83.417 | 1.00 | 0.00 | xxxx | 1321 |
| ATOM | 1322 | CG | LYS | A | 172 | 56.199 | 15.537 | 82.261 | 1.00 | 0.00 | xxxx | 1322 |
| ATOM | 1323 | CD | LYS | A | 172 | 55.181 | 16.549 | 82.783 | 1.00 | 0.00 | xxxx | 1323 |
| ATOM | 1324 | CE | LYS | A | 172 | 54.534 | 17.326 | 81.650 | 1.00 | 0.00 | xxxx | 1324 |
| ATOM | 1325 | NZ | LYS | A | 172 | 55.536 | 18.060 | 80.827 | 1.00 | 0.00 | xxxx | 1325 |
| ATOM | 1326 | N | THR | A | 173 | 59.548 | 13.723 | 84.672 | 1.00 | 0.00 | xxxx | 1326 |
| ATOM | 1327 | CA | THR | A | 173 | 60.320 | 13.197 | 85.792 | 1.00 | 0.00 | xxxx | 1327 |
| ATOM | 1328 | C | THR | A | 173 | 60.618 | 14.325 | 86.761 | 1.00 | 0.00 | xxxx | 1328 |
| ATOM | 1329 | O | THR | A | 173 | 60.759 | 15.480 | 86.353 | 1.00 | 0.00 | xxxx | 1329 |
| ATOM | 1330 | CB | THR | A | 173 | 61.635 | 12.548 | 85.320 | 1.00 | 0.00 | xxxx | 1330 |
| ATOM | 1331 | OG1 | THR | A | 173 | 62.379 | 13.472 | 84.508 | 1.00 | 0.00 | xxxx | 1331 |
| ATOM | 1332 | CG2 | THR | A | 173 | 61.352 | 11.291 | 84.503 | 1.00 | 0.00 | xxxx | 1332 |
| ATOM | 1333 | N | GLU | A | 174 | 60.673 | 13.995 | 88.047 | 1.00 | 0.00 | xxxx | 1333 |
| ATOM | 1334 | CA | GLU | A | 174 | 61.057 | 14.966 | 89.058 | 1.00 | 0.00 | xxxx | 1334 |
| ATOM | 1335 | C | GLU | A | 174 | 62.205 | 14.408 | 89.878 | 1.00 | 0.00 | xxxx | 1335 |
| ATOM | 1336 | O | GLU | A | 174 | 62.055 | 13.407 | 90.580 | 1.00 | 0.00 | xxxx | 1336 |
| ATOM | 1337 | CB | GLU | A | 174 | 59.894 | 15.330 | 89.985 | 1.00 | 0.00 | xxxx | 1337 |
| ATOM | 1338 | CG | GLU | A | 174 | 60.320 | 16.389 | 91.020 | 1.00 | 0.00 | xxxx | 1338 |
| ATOM | 1339 | CD | GLU | A | 174 | 59.264 | 16.733 | 92.064 | 1.00 | 0.00 | xxxx | 1339 |
| ATOM | 1340 | OE1 | GLU | A | 174 | 58.196 | 16.086 | 92.123 | 1.00 | 0.00 | xxxx | 1340 |
| ATOM | 1341 | OE2 | GLU | A | 174 | 59.516 | 17.671 | 92.848 | 1.00 | 0.00 | xxxx | 1341 |
| ATOM | 1342 | N | GLN | A | 175 | 63.350 | 15.074 | 89.787 | 1.00 | 0.00 | xxxx | 1342 |
| ATOM | 1343 | CA | GLN | A | 175 | 64.556 | 14.661 | 90.510 | 1.00 | 0.00 | xxxx | 1343 |
| ATOM | 1344 | C | GLN | A | 175 | 64.469 | 15.050 | 91.980 | 1.00 | 0.00 | xxxx | 1344 |
| ATOM | 1345 | O | GLN | A | 175 | 64.574 | 16.234 | 92.315 | 1.00 | 0.00 | xxxx | 1345 |
| ATOM | 1346 | CB | GLN | A | 175 | 65.783 | 15.304 | 89.874 | 1.00 | 0.00 | xxxx | 1346 |
| ATOM | 1347 | CG | GLN | A | 175 | 67.103 | 14.879 | 90.484 | 1.00 | 0.00 | xxxx | 1347 |
| ATOM | 1348 | CD | GLN | A | 175 | 68.250 | 15.684 | 89.923 | 1.00 | 0.00 | xxxx | 1348 |
| ATOM | 1349 | OE1 | GLN | A | 175 | 68.903 | 16.442 | 90.641 | 1.00 | 0.00 | xxxx | 1349 |
| ATOM | 1350 | NE2 | GLN | A | 175 | 68.483 | 15.545 | 88.629 | 1.00 | 0.00 | xxxx | 1350 |
| ATOM | 1351 | N | LEU | A | 176 | 64.262 | 14.067 | 92.850 | 1.00 | 0.00 | xxxx | 1351 |
| ATOM | 1352 | CA | LEU | A | 176 | 64.183 | 14.350 | 94.293 | 1.00 | 0.00 | xxxx | 1352 |
| ATOM | 1353 | C | LEU | A | 176 | 65.546 | 14.387 | 94.955 | 1.00 | 0.00 | xxxx | 1353 |
| ATOM | 1354 | O | LEU | A | 176 | 65.767 | 15.180 | 95.866 | 1.00 | 0.00 | xxxx | 1354 |
| ATOM | 1355 | CB | LEU | A | 176 | 63.312 | 13.305 | 94.999 | 1.00 | 0.00 | xxxx | 1355 |
| ATOM | 1356 | CG | LEU | A | 176 | 61.856 | 13.217 | 94.549 | 1.00 | 0.00 | xxxx | 1356 |
| ATOM | 1357 | CD1 | LEU | A | 176 | 61.175 | 12.071 | 95.308 | 1.00 | 0.00 | xxxx | 1357 |
| ATOM | 1358 | CD2 | LEU | A | 176 | 61.139 | 14.546 | 94.788 | 1.00 | 0.00 | xxxx | 1358 |
| ATOM | 1359 | N | GLN | A | 177 | 66.435 | 13.483 | 94.545 | 1.00 | 0.00 | xxxx | 1359 |
| ATOM | 1360 | CA | GLN | A | 177 | 67.817 | 13.462 | 95.029 | 1.00 | 0.00 | xxxx | 1360 |
| ATOM | 1361 | C | GLN | A | 177 | 68.693 | 12.943 | 93.896 | 1.00 | 0.00 | xxxx | 1361 |
| ATOM | 1362 | O | GLN | A | 177 | 68.228 | 12.150 | 93.071 | 1.00 | 0.00 | xxxx | 1362 |
| ATOM | 1363 | CB | GLN | A | 177 | 68.011 | 12.551 | 96.262 | 1.00 | 0.00 | xxxx | 1363 |
| ATOM | 1364 | CG | GLN | A | 177 | 66.981 | 12.631 | 97.393 | 1.00 | 0.00 | xxxx | 1364 |
| ATOM | 1365 | CD | GLN | A | 177 | 67.126 | 13.831 | 98.332 | 1.00 | 0.00 | xxxx | 1365 |
| ATOM | 1366 | OE1 | GLN | A | 177 | 66.270 | 14.034 | 99.194 | 1.00 | 0.00 | xxxx | 1366 |
| ATOM | 1367 | NE2 | GLN | A | 177 | 68.204 | 14.605 | 98.189 | 1.00 | 0.00 | xxxx | 1367 |
| ATOM | 1368 | N | LEU | A | 178 | 69.944 | 13.386 | 93.865 | 1.00 | 0.00 | xxxx | 1368 |
| ATOM | 1369 | CA | LEU | A | 178 | 70.942 | 12.869 | 92.930 | 1.00 | 0.00 | xxxx | 1369 |
| ATOM | 1370 | C | LEU | A | 178 | 72.319 | 13.073 | 93.530 | 1.00 | 0.00 | xxxx | 1370 |
| ATOM | 1371 | O | LEU | A | 178 | 72.658 | 14.177 | 93.954 | 1.00 | 0.00 | xxxx | 1371 |
| ATOM | 1372 | CB | LEU | A | 178 | 70.848 | 13.555 | 91.561 | 1.00 | 0.00 | xxxx | 1372 |
| ATOM | 1373 | CG | LEU | A | 178 | 71.737 | 12.993 | 90.440 | 1.00 | 0.00 | xxxx | 1373 |
| ATOM | 1374 | CD1 | LEU | A | 178 | 71.094 | 13.273 | 89.093 | 1.00 | 0.00 | xxxx | 1374 |
| ATOM | 1375 | CD2 | LEU | A | 178 | 73.146 | 13.573 | 90.458 | 1.00 | 0.00 | xxxx | 1375 |
| ATOM | 1376 | N | ASP | A | 179 | 73.110 | 12.011 | 93.565 | 1.00 | 0.00 | xxxx | 1376 |
| ATOM | 1377 | CA | ASP | A | 179 | 74.449 | 12.118 | 94.146 | 1.00 | 0.00 | xxxx | 1377 |
| ATOM | 1378 | C | ASP | A | 179 | 75.281 | 10.939 | 93.713 | 1.00 | 0.00 | xxxx | 1378 |
| ATOM | 1379 | O | ASP | A | 179 | 74.745 | 9.903 | 93.320 | 1.00 | 0.00 | xxxx | 1379 |
| ATOM | 1380 | CB | ASP | A | 179 | 74.373 | 12.174 | 95.674 | 1.00 | 0.00 | xxxx | 1380 |
| ATOM | 1381 | CG | ASP | A | 179 | 75.519 | 12.961 | 96.307 | 1.00 | 0.00 | xxxx | 1381 |
| ATOM | 1382 | OD1 | ASP | A | 179 | 76.586 | 13.168 | 95.674 | 1.00 | 0.00 | xxxx | 1382 |
| ATOM | 1383 | OD2 | ASP | A | 179 | 75.343 | 13.385 | 97.470 | 1.00 | 0.00 | xxxx | 1383 |
| ATOM | 1384 | N | THR | A | 180 | 76.599 | 11.086 | 93.801 | 1.00 | 0.00 | xxxx | 1384 |
| ATOM | 1385 | CA | THR | A | 180 | 77.473 | 9.975 | 93.475 | 1.00 | 0.00 | xxxx | 1385 |
| ATOM | 1386 | C | THR | A | 180 | 77.575 | 9.026 | 94.662 | 1.00 | 0.00 | xxxx | 1386 |
| ATOM | 1387 | O | THR | A | 180 | 77.531 | 9.442 | 95.826 | 1.00 | 0.00 | xxxx | 1387 |
| ATOM | 1388 | CB | THR | A | 180 | 78.880 | 10.457 | 93.069 | 1.00 | 0.00 | xxxx | 1388 |
| ATOM | 1389 | OG1 | THR | A | 180 | 79.674 | 9.327 | 92.674 | 1.00 | 0.00 | xxxx | 1389 |
| ATOM | 1390 | CG2 | THR | A | 180 | 79.568 | 11.183 | 94.230 | 1.00 | 0.00 | xxxx | 1390 |
| ATOM | 1391 | N | ALA | A | 181 | 77.682 | 7.739 | 94.364 | 1.00 | 0.00 | xxxx | 1391 |
| ATOM | 1392 | CA | ALA | A | 181 | 78.063 | 6.770 | 95.369 | 1.00 | 0.00 | xxxx | 1392 |
| ATOM | 1393 | C | ALA | A | 181 | 79.237 | 5.967 | 94.826 | 1.00 | 0.00 | xxxx | 1393 |
| ATOM | 1394 | O | ALA | A | 181 | 79.492 | 4.850 | 95.269 | 1.00 | 0.00 | xxxx | 1394 |
| ATOM | 1395 | CB | ALA | A | 181 | 76.890 | 5.868 | 95.738 | 1.00 | 0.00 | xxxx | 1395 |
| ATOM | 1396 | N | MET | A | 182 | 79.941 | 6.564 | 93.859 | 1.00 | 0.00 | xxxx | 1396 |
| ATOM | 1397 | CA | MET | A | 182 | 81.259 | 6.101 | 93.418 | 1.00 | 0.00 | xxxx | 1397 |
| ATOM | 1398 | C | MET | A | 182 | 81.306 | 4.625 | 93.052 | 1.00 | 0.00 | xxxx | 1398 |
| ATOM | 1399 | O | MET | A | 182 | 82.284 | 3.931 | 93.358 | 1.00 | 0.00 | xxxx | 1399 |
| ATOM | 1400 | CB | MET | A | 182 | 82.300 | 6.394 | 94.501 | 1.00 | 0.00 | xxxx | 1400 |
| ATOM | 1401 | CG | MET | A | 182 | 82.438 | 7.896 | 94.802 | 1.00 | 0.00 | xxxx | 1401 |
| ATOM | 1402 | SD | MET | A | 182 | 83.054 | 8.899 | 93.419 | 1.00 | 0.00 | xxxx | 1402 |
| ATOM | 1403 | CE | MET | A | 182 | 84.811 | 8.596 | 93.549 | 1.00 | 0.00 | xxxx | 1403 |
| ATOM | 1404 | N | CYS | A | 183 | 80.232 | 4.159 | 92.419 | 1.00 | 0.00 | xxxx | 1404 |
| ATOM | 1405 | CA | CYS | A | 183 | 80.152 | 2.815 | 91.855 | 1.00 | 0.00 | xxxx | 1405 |
| ATOM | 1406 | C | CYS | A | 183 | 80.215 | 1.715 | 92.915 | 1.00 | 0.00 | xxxx | 1406 |
| ATOM | 1407 | O | CYS | A | 183 | 80.418 | 0.555 | 92.578 | 1.00 | 0.00 | xxxx | 1407 |
| ATOM | 1408 | CB | CYS | A | 183 | 81.268 | 2.616 | 90.816 | 1.00 | 0.00 | xxxx | 1408 |
| ATOM | 1409 | SG | CYS | A | 183 | 81.332 | 4.000 | 89.637 | 1.00 | 0.00 | xxxx | 1409 |
| ATOM | 1410 | N | ASP | A | 184 | 79.976 | 2.079 | 94.179 | 1.00 | 0.00 | xxxx | 1410 |
| ATOM | 1411 | CA | ASP | A | 184 | 80.214 | 1.203 | 95.330 | 1.00 | 0.00 | xxxx | 1411 |
| ATOM | 1412 | C | ASP | A | 184 | 78.923 | 0.838 | 96.079 | 1.00 | 0.00 | xxxx | 1412 |
| ATOM | 1413 | O | ASP | A | 184 | 78.117 | 1.714 | 96.375 | 1.00 | 0.00 | xxxx | 1413 |
| ATOM | 1414 | CB | ASP | A | 184 | 81.210 | 1.895 | 96.281 | 1.00 | 0.00 | xxxx | 1414 |
| ATOM | 1415 | CG | ASP | A | 184 | 81.539 | 1.069 | 97.504 | 1.00 | 0.00 | xxxx | 1415 |
| ATOM | 1416 | OD1 | ASP | A | 184 | 82.395 | 0.168 | 97.414 | 1.00 | 0.00 | xxxx | 1416 |
| ATOM | 1417 | OD2 | ASP | A | 184 | 80.969 | 1.338 | 98.574 | 1.00 | 0.00 | xxxx | 1417 |
| ATOM | 1418 | N | THR | A | 185 | 78.740 | −0.444 | 96.401 | 1.00 | 0.00 | xxxx | 1418 |
| ATOM | 1419 | CA | THR | A | 185 | 77.520 | −0.913 | 97.077 | 1.00 | 0.00 | xxxx | 1419 |
| ATOM | 1420 | C | THR | A | 185 | 77.309 | −0.291 | 98.464 | 1.00 | 0.00 | xxxx | 1420 |
| ATOM | 1421 | O | THR | A | 185 | 76.217 | 0.206 | 98.768 | 1.00 | 0.00 | xxxx | 1421 |
| ATOM | 1422 | CB | THR | A | 185 | 77.519 | −2.460 | 97.230 | 1.00 | 0.00 | xxxx | 1422 |
| ATOM | 1423 | OG1 | THR | A | 185 | 77.522 | −3.072 | 95.931 | 1.00 | 0.00 | xxxx | 1423 |
| ATOM | 1424 | CG2 | THR | A | 185 | 76.289 | −2.934 | 97.982 | 1.00 | 0.00 | xxxx | 1424 |
| ATOM | 1425 | N | ALA | A | 186 | 78.331 | −0.330 | 99.312 | 1.00 | 0.00 | xxxx | 1425 |
| ATOM | 1426 | CA | ALA | A | 186 | 78.187 | 0.212 | 100.664 | 1.00 | 0.00 | xxxx | 1426 |
| ATOM | 1427 | C | ALA | A | 186 | 77.948 | 1.725 | 100.657 | 1.00 | 0.00 | xxxx | 1427 |
| ATOM | 1428 | O | ALA | A | 186 | 77.117 | 2.229 | 101.429 | 1.00 | 0.00 | xxxx | 1428 |
| ATOM | 1429 | CB | ALA | A | 186 | 79.412 | −0.129 | 101.507 | 1.00 | 0.00 | xxxx | 1429 |
| ATOM | 1430 | N | GLN | A | 187 | 78.647 | 2.454 | 99.792 | 1.00 | 0.00 | xxxx | 1430 |
| ATOM | 1431 | CA | GLN | A | 187 | 78.406 | 3.895 | 99.687 | 1.00 | 0.00 | xxxx | 1431 |
| ATOM | 1432 | C | GLN | A | 187 | 76.977 | 4.178 | 99.226 | 1.00 | 0.00 | xxxx | 1432 |
| ATOM | 1433 | O | GLN | A | 187 | 76.335 | 5.126 | 99.694 | 1.00 | 0.00 | xxxx | 1433 |
| ATOM | 1434 | CB | GLN | A | 187 | 79.390 | 4.562 | 98.725 | 1.00 | 0.00 | xxxx | 1434 |
| ATOM | 1435 | CG | GLN | A | 187 | 80.818 | 4.676 | 99.221 | 1.00 | 0.00 | xxxx | 1435 |
| ATOM | 1436 | CD | GLN | A | 187 | 81.613 | 5.683 | 98.408 | 1.00 | 0.00 | xxxx | 1436 |
| ATOM | 1437 | OE1 | GLN | A | 187 | 81.082 | 6.715 | 97.987 | 1.00 | 0.00 | xxxx | 1437 |
| ATOM | 1438 | NE2 | GLN | A | 187 | 82.885 | 5.385 | 98.173 | 1.00 | 0.00 | xxxx | 1438 |
| ATOM | 1439 | N | ALA | A | 188 | 76.470 | 3.356 | 98.313 | 1.00 | 0.00 | xxxx | 1439 |
| ATOM | 1440 | CA | ALA | A | 188 | 75.100 | 3.522 | 97.820 | 1.00 | 0.00 | xxxx | 1440 |
| ATOM | 1441 | C | ALA | A | 188 | 74.053 | 3.210 | 98.891 | 1.00 | 0.00 | xxxx | 1441 |
| ATOM | 1442 | O | ALA | A | 188 | 73.034 | 3.888 | 98.979 | 1.00 | 0.00 | xxxx | 1442 |
| ATOM | 1443 | CB | ALA | A | 188 | 74.877 | 2.642 | 96.604 | 1.00 | 0.00 | xxxx | 1443 |
| ATOM | 1444 | N | LYS | A | 189 | 74.302 | 2.173 | 99.690 | 1.00 | 0.00 | xxxx | 1444 |
| ATOM | 1445 | CA | LYS | A | 189 | 73.401 | 1.832 | 100.789 | 1.00 | 0.00 | xxxx | 1445 |
| ATOM | 1446 | C | LYS | A | 189 | 73.337 | 3.005 | 101.764 | 1.00 | 0.00 | xxxx | 1446 |
| ATOM | 1447 | O | LYS | A | 189 | 72.250 | 3.385 | 102.219 | 1.00 | 0.00 | xxxx | 1447 |
| ATOM | 1448 | CB | LYS | A | 189 | 73.860 | 0.551 | 101.506 | 1.00 | 0.00 | xxxx | 1448 |
| ATOM | 1449 | CG | LYS | A | 189 | 72.930 | 0.137 | 102.642 | 1.00 | 0.00 | xxxx | 1449 |
| ATOM | 1450 | CD | LYS | A | 189 | 73.420 | −1.084 | 103.397 | 1.00 | 0.00 | xxxx | 1450 |
| ATOM | 1451 | CE | LYS | A | 189 | 72.458 | −1.431 | 104.524 | 1.00 | 0.00 | xxxx | 1451 |
| ATOM | 1452 | NZ | LYS | A | 189 | 72.884 | −2.636 | 105.292 | 1.00 | 0.00 | xxxx | 1452 |
| ATOM | 1453 | N | ASP | A | 190 | 74.502 | 3.586 | 102.066 | 1.00 | 0.00 | xxxx | 1453 |
| ATOM | 1454 | CA | ASP | A | 190 | 74.553 | 4.737 | 102.971 | 1.00 | 0.00 | xxxx | 1454 |
| ATOM | 1455 | C | ASP | A | 190 | 73.784 | 5.927 | 102.397 | 1.00 | 0.00 | xxxx | 1455 |
| ATOM | 1456 | O | ASP | A | 190 | 73.018 | 6.591 | 103.106 | 1.00 | 0.00 | xxxx | 1456 |
| ATOM | 1457 | CB | ASP | A | 190 | 75.996 | 5.149 | 103.260 | 1.00 | 0.00 | xxxx | 1457 |
| ATOM | 1458 | CG | ASP | A | 190 | 76.070 | 6.413 | 104.082 | 1.00 | 0.00 | xxxx | 1458 |
| ATOM | 1459 | OD1 | ASP | A | 190 | 75.855 | 6.332 | 105.307 | 1.00 | 0.00 | xxxx | 1459 |
| ATOM | 1460 | OD2 | ASP | A | 190 | 76.329 | 7.489 | 103.500 | 1.00 | 0.00 | xxxx | 1460 |
| ATOM | 1461 | N | LYS | A | 191 | 73.983 | 6.197 | 101.113 | 1.00 | 0.00 | xxxx | 1461 |
| ATOM | 1462 | CA | LYS | A | 191 | 73.330 | 7.344 | 100.479 | 1.00 | 0.00 | xxxx | 1462 |
| ATOM | 1463 | C | LYS | A | 191 | 71.811 | 7.147 | 100.415 | 1.00 | 0.00 | xxxx | 1463 |
| ATOM | 1464 | O | LYS | A | 191 | 71.035 | 8.050 | 100.762 | 1.00 | 0.00 | xxxx | 1464 |
| ATOM | 1465 | CB | LYS | A | 191 | 73.908 | 7.553 | 99.072 | 1.00 | 0.00 | xxxx | 1465 |
| ATOM | 1466 | CG | LYS | A | 191 | 73.374 | 8.747 | 98.322 | 1.00 | 0.00 | xxxx | 1466 |
| ATOM | 1467 | CD | LYS | A | 191 | 73.720 | 10.087 | 98.980 | 1.00 | 0.00 | xxxx | 1467 |
| ATOM | 1468 | CE | LYS | A | 191 | 75.228 | 10.258 | 99.200 | 1.00 | 0.00 | xxxx | 1468 |
| ATOM | 1469 | NZ | LYS | A | 191 | 75.550 | 11.651 | 99.690 | 1.00 | 0.00 | xxxx | 1469 |
| ATOM | 1470 | N | MET | A | 192 | 71.391 | 5.958 | 99.994 | 1.00 | 0.00 | xxxx | 1470 |
| ATOM | 1471 | CA | MET | A | 192 | 69.970 | 5.658 | 99.924 | 1.00 | 0.00 | xxxx | 1471 |
| ATOM | 1472 | C | MET | A | 192 | 69.313 | 5.698 | 101.304 | 1.00 | 0.00 | xxxx | 1472 |
| ATOM | 1473 | O | MET | A | 192 | 68.208 | 6.231 | 101.459 | 1.00 | 0.00 | xxxx | 1473 |
| ATOM | 1474 | CB | MET | A | 192 | 69.752 | 4.289 | 99.270 | 1.00 | 0.00 | xxxx | 1474 |
| ATOM | 1475 | CG | MET | A | 192 | 68.289 | 3.992 | 98.975 | 1.00 | 0.00 | xxxx | 1475 |
| ATOM | 1476 | SD | MET | A | 192 | 67.643 | 5.014 | 97.628 | 1.00 | 0.00 | xxxx | 1476 |
| ATOM | 1477 | CE | MET | A | 192 | 65.900 | 4.966 | 98.028 | 1.00 | 0.00 | xxxx | 1477 |
| ATOM | 1478 | N | ASP | A | 193 | 69.997 | 5.149 | 102.305 | 1.00 | 0.00 | xxxx | 1478 |
| ATOM | 1479 | CA | ASP | A | 193 | 69.495 | 5.202 | 103.671 | 1.00 | 0.00 | xxxx | 1479 |
| ATOM | 1480 | C | ASP | A | 193 | 69.318 | 6.653 | 104.133 | 1.00 | 0.00 | xxxx | 1480 |
| ATOM | 1481 | O | ASP | A | 193 | 68.323 | 6.989 | 104.793 | 1.00 | 0.00 | xxxx | 1481 |
| ATOM | 1482 | CB | ASP | A | 193 | 70.432 | 4.460 | 104.621 | 1.00 | 0.00 | xxxx | 1482 |
| ATOM | 1483 | CG | ASP | A | 193 | 69.987 | 4.572 | 106.057 | 1.00 | 0.00 | xxxx | 1483 |
| ATOM | 1484 | OD1 | ASP | A | 193 | 70.516 | 5.447 | 106.762 | 1.00 | 0.00 | xxxx | 1484 |
| ATOM | 1485 | OD2 | ASP | A | 193 | 69.077 | 3.814 | 106.469 | 1.00 | 0.00 | xxxx | 1485 |
| ATOM | 1486 | N | ALA | A | 194 | 70.274 | 7.516 | 103.797 | 1.00 | 0.00 | xxxx | 1486 |
| ATOM | 1487 | CA | ALA | A | 194 | 70.134 | 8.929 | 104.139 | 1.00 | 0.00 | xxxx | 1487 |
| ATOM | 1488 | C | ALA | A | 194 | 68.870 | 9.526 | 103.503 | 1.00 | 0.00 | xxxx | 1488 |
| ATOM | 1489 | O | ALA | A | 194 | 68.095 | 10.221 | 104.167 | 1.00 | 0.00 | xxxx | 1489 |
| ATOM | 1490 | CB | ALA | A | 194 | 71.377 | 9.712 | 103.703 | 1.00 | 0.00 | xxxx | 1490 |
| ATOM | 1491 | N | TRP | A | 195 | 68.656 | 9.239 | 102.224 | 1.00 | 0.00 | xxxx | 1491 |
| ATOM | 1492 | CA | TRP | A | 195 | 67.502 | 9.766 | 101.511 | 1.00 | 0.00 | xxxx | 1492 |
| ATOM | 1493 | C | TRP | A | 195 | 66.187 | 9.244 | 102.097 | 1.00 | 0.00 | xxxx | 1493 |
| ATOM | 1494 | O | TRP | A | 195 | 65.198 | 9.973 | 102.188 | 1.00 | 0.00 | xxxx | 1494 |
| ATOM | 1495 | CB | TRP | A | 195 | 67.624 | 9.412 | 100.034 | 1.00 | 0.00 | xxxx | 1495 |
| ATOM | 1496 | CG | TRP | A | 195 | 68.764 | 10.131 | 99.346 | 1.00 | 0.00 | xxxx | 1496 |
| ATOM | 1497 | CD1 | TRP | A | 195 | 69.427 | 11.225 | 99.801 | 1.00 | 0.00 | xxxx | 1497 |
| ATOM | 1498 | CD2 | TRP | A | 195 | 69.350 | 9.802 | 98.079 | 1.00 | 0.00 | xxxx | 1498 |
| ATOM | 1499 | NE1 | TRP | A | 195 | 70.403 | 11.602 | 98.894 | 1.00 | 0.00 | xxxx | 1499 |
| ATOM | 1500 | CE2 | TRP | A | 195 | 70.374 | 10.742 | 97.833 | 1.00 | 0.00 | xxxx | 1500 |
| ATOM | 1501 | CE3 | TRP | A | 195 | 69.116 | 8.799 | 97.135 | 1.00 | 0.00 | xxxx | 1501 |
| ATOM | 1502 | CZ2 | TRP | A | 195 | 71.149 | 10.719 | 96.668 | 1.00 | 0.00 | xxxx | 1502 |
| ATOM | 1503 | CZ3 | TRP | A | 195 | 69.898 | 8.779 | 95.971 | 1.00 | 0.00 | xxxx | 1503 |
| ATOM | 1504 | CH2 | TRP | A | 195 | 70.892 | 9.733 | 95.755 | 1.00 | 0.00 | xxxx | 1504 |
| ATOM | 1505 | N | LEU | A | 196 | 66.189 | 7.980 | 102.514 | 1.00 | 0.00 | xxxx | 1505 |
| ATOM | 1506 | CA | LEU | A | 196 | 65.022 | 7.406 | 103.164 | 1.00 | 0.00 | xxxx | 1506 |
| ATOM | 1507 | C | LEU | A | 196 | 64.808 | 7.957 | 104.566 | 1.00 | 0.00 | xxxx | 1507 |
| ATOM | 1508 | O | LEU | A | 196 | 63.775 | 7.692 | 105.177 | 1.00 | 0.00 | xxxx | 1508 |
| ATOM | 1509 | CB | LEU | A | 196 | 65.141 | 5.879 | 103.217 | 1.00 | 0.00 | xxxx | 1509 |
| ATOM | 1510 | CG | LEU | A | 196 | 65.043 | 5.236 | 101.832 | 1.00 | 0.00 | xxxx | 1510 |
| ATOM | 1511 | CD1 | LEU | A | 196 | 65.477 | 3.771 | 101.881 | 1.00 | 0.00 | xxxx | 1511 |
| ATOM | 1512 | CD2 | LEU | A | 196 | 63.614 | 5.360 | 101.304 | 1.00 | 0.00 | xxxx | 1512 |
| ATOM | 1513 | N | SER | A | 197 | 65.792 | 8.703 | 105.073 | 1.00 | 0.00 | xxxx | 1513 |
| ATOM | 1514 | CA | SER | A | 197 | 65.721 | 9.321 | 106.390 | 1.00 | 0.00 | xxxx | 1514 |
| ATOM | 1515 | C | SER | A | 197 | 65.516 | 10.830 | 106.277 | 1.00 | 0.00 | xxxx | 1515 |
| ATOM | 1516 | O | SER | A | 197 | 65.524 | 11.527 | 107.294 | 1.00 | 0.00 | xxxx | 1516 |
| ATOM | 1517 | CB | SER | A | 197 | 66.992 | 9.043 | 107.198 | 1.00 | 0.00 | xxxx | 1517 |
| ATOM | 1518 | OG | SER | A | 197 | 67.260 | 7.649 | 107.316 | 1.00 | 0.00 | xxxx | 1518 |
| ATOM | 1519 | N | GLY | A | 198 | 65.321 | 11.322 | 105.050 | 1.00 | 0.00 | xxxx | 1519 |
| ATOM | 1520 | CA | GLY | A | 198 | 65.148 | 12.751 | 104.807 | 1.00 | 0.00 | xxxx | 1520 |
| ATOM | 1521 | C | GLY | A | 198 | 63.742 | 13.078 | 104.341 | 1.00 | 0.00 | xxxx | 1521 |
| ATOM | 1522 | O | GLY | A | 198 | 62.900 | 12.186 | 104.216 | 1.00 | 0.00 | xxxx | 1522 |
| ATOM | 1523 | N | PRO | A | 199 | 63.478 | 14.363 | 104.074 | 1.00 | 0.00 | xxxx | 1523 |
| ATOM | 1524 | CA | PRO | A | 199 | 62.106 | 14.833 | 103.839 | 1.00 | 0.00 | xxxx | 1524 |
| ATOM | 1525 | C | PRO | A | 199 | 61.464 | 14.338 | 102.545 | 1.00 | 0.00 | xxxx | 1525 |
| ATOM | 1526 | O | PRO | A | 199 | 60.239 | 14.456 | 102.417 | 1.00 | 0.00 | xxxx | 1526 |
| ATOM | 1527 | CB | PRO | A | 199 | 62.254 | 16.364 | 103.800 | 1.00 | 0.00 | xxxx | 1527 |
| ATOM | 1528 | CG | PRO | A | 199 | 63.581 | 16.657 | 104.428 | 1.00 | 0.00 | xxxx | 1528 |
| ATOM | 1529 | CD | PRO | A | 199 | 64.450 | 15.464 | 104.162 | 1.00 | 0.00 | xxxx | 1529 |
| ATOM | 1530 | N | ASN | A | 200 | 62.245 | 13.815 | 101.604 | 1.00 | 0.00 | xxxx | 1530 |
| ATOM | 1531 | CA | ASN | A | 200 | 61.655 | 13.290 | 100.370 | 1.00 | 0.00 | xxxx | 1531 |
| ATOM | 1532 | C | ASN | A | 200 | 61.381 | 11.791 | 100.423 | 1.00 | 0.00 | xxxx | 1532 |
| ATOM | 1533 | O | ASN | A | 200 | 60.945 | 11.220 | 99.420 | 1.00 | 0.00 | xxxx | 1533 |
| ATOM | 1534 | CB | ASN | A | 200 | 62.557 | 13.582 | 99.166 | 1.00 | 0.00 | xxxx | 1534 |
| ATOM | 1535 | CG | ASN | A | 200 | 62.652 | 15.068 | 98.858 | 1.00 | 0.00 | xxxx | 1535 |
| ATOM | 1536 | OD1 | ASN | A | 200 | 61.657 | 15.795 | 98.911 | 1.00 | 0.00 | xxxx | 1536 |
| ATOM | 1537 | ND2 | ASN | A | 200 | 63.854 | 15.527 | 98.550 | 1.00 | 0.00 | xxxx | 1537 |
| ATOM | 1538 | N | ALA | A | 201 | 61.644 | 11.147 | 101.563 | 1.00 | 0.00 | xxxx | 1538 |
| ATOM | 1539 | CA | ALA | A | 201 | 61.533 | 9.689 | 101.645 | 1.00 | 0.00 | xxxx | 1539 |
| ATOM | 1540 | C | ALA | A | 201 | 60.185 | 9.168 | 101.122 | 1.00 | 0.00 | xxxx | 1540 |
| ATOM | 1541 | O | ALA | A | 201 | 60.157 | 8.216 | 100.329 | 1.00 | 0.00 | xxxx | 1541 |
| ATOM | 1542 | CB | ALA | A | 201 | 61.745 | 9.228 | 103.059 | 1.00 | 0.00 | xxxx | 1542 |
| ATOM | 1543 | N | ASN | A | 202 | 59.097 | 9.813 | 101.547 | 1.00 | 0.00 | xxxx | 1543 |
| ATOM | 1544 | CA | ASN | A | 202 | 57.746 | 9.334 | 101.237 | 1.00 | 0.00 | xxxx | 1544 |
| ATOM | 1545 | C | ASN | A | 202 | 57.364 | 9.580 | 99.785 | 1.00 | 0.00 | xxxx | 1545 |
| ATOM | 1546 | O | ASN | A | 202 | 56.394 | 8.997 | 99.291 | 1.00 | 0.00 | xxxx | 1546 |
| ATOM | 1547 | CB | ASN | A | 202 | 56.705 | 9.988 | 102.165 | 1.00 | 0.00 | xxxx | 1547 |
| ATOM | 1548 | CG | ASN | A | 202 | 56.603 | 11.501 | 101.979 | 1.00 | 0.00 | xxxx | 1548 |
| ATOM | 1549 | OD1 | ASN | A | 202 | 57.530 | 12.243 | 102.302 | 1.00 | 0.00 | xxxx | 1549 |
| ATOM | 1550 | ND2 | ASN | A | 202 | 55.465 | 11.961 | 101.474 | 1.00 | 0.00 | xxxx | 1550 |
| ATOM | 1551 | N | LYS | A | 203 | 58.134 | 10.428 | 99.110 | 1.00 | 0.00 | xxxx | 1551 |
| ATOM | 1552 | CA | LYS | A | 203 | 57.851 | 10.816 | 97.730 | 1.00 | 0.00 | xxxx | 1552 |
| ATOM | 1553 | C | LYS | A | 203 | 58.576 | 9.978 | 96.686 | 1.00 | 0.00 | xxxx | 1553 |
| ATOM | 1554 | O | LYS | A | 203 | 58.195 | 10.012 | 95.514 | 1.00 | 0.00 | xxxx | 1554 |
| ATOM | 1555 | CB | LYS | A | 203 | 58.218 | 12.287 | 97.509 | 1.00 | 0.00 | xxxx | 1555 |
| ATOM | 1556 | CG | LYS | A | 203 | 57.499 | 13.250 | 98.431 | 1.00 | 0.00 | xxxx | 1556 |
| ATOM | 1557 | CD | LYS | A | 203 | 57.973 | 14.674 | 98.189 | 1.00 | 0.00 | xxxx | 1557 |
| ATOM | 1558 | CE | LYS | A | 203 | 58.639 | 15.242 | 99.431 | 1.00 | 0.00 | xxxx | 1558 |
| ATOM | 1559 | NZ | LYS | A | 203 | 57.791 | 15.088 | 100.650 | 1.00 | 0.00 | xxxx | 1559 |
| ATOM | 1560 | N | ILE | A | 204 | 59.623 | 9.250 | 97.090 | 1.00 | 0.00 | xxxx | 1560 |
| ATOM | 1561 | CA | ILE | A | 204 | 60.423 | 8.457 | 96.146 | 1.00 | 0.00 | xxxx | 1561 |
| ATOM | 1562 | C | ILE | A | 204 | 59.602 | 7.349 | 95.500 | 1.00 | 0.00 | xxxx | 1562 |
| ATOM | 1563 | O | ILE | A | 204 | 59.039 | 6.510 | 96.207 | 1.00 | 0.00 | xxxx | 1563 |
| ATOM | 1564 | CB | ILE | A | 204 | 61.645 | 7.841 | 96.839 | 1.00 | 0.00 | xxxx | 1564 |
| ATOM | 1565 | CG1 | ILE | A | 204 | 62.522 | 8.943 | 97.425 | 1.00 | 0.00 | xxxx | 1565 |
| ATOM | 1566 | CD1 | ILE | A | 204 | 63.655 | 8.425 | 98.336 | 1.00 | 0.00 | xxxx | 1566 |
| ATOM | 1567 | CG2 | ILE | A | 204 | 62.435 | 6.991 | 95.839 | 1.00 | 0.00 | xxxx | 1567 |
| ATOM | 1568 | N | GLU | A | 205 | 59.555 | 7.333 | 94.165 | 1.00 | 0.00 | xxxx | 1568 |
| ATOM | 1569 | CA | GLU | A | 205 | 58.783 | 6.322 | 93.451 | 1.00 | 0.00 | xxxx | 1569 |
| ATOM | 1570 | C | GLU | A | 205 | 59.667 | 5.317 | 92.719 | 1.00 | 0.00 | xxxx | 1570 |
| ATOM | 1571 | O | GLU | A | 205 | 59.304 | 4.147 | 92.612 | 1.00 | 0.00 | xxxx | 1571 |
| ATOM | 1572 | CB | GLU | A | 205 | 57.816 | 6.995 | 92.459 | 1.00 | 0.00 | xxxx | 1572 |
| ATOM | 1573 | CG | GLU | A | 205 | 56.791 | 7.866 | 93.156 | 1.00 | 0.00 | xxxx | 1573 |
| ATOM | 1574 | CD | GLU | A | 205 | 55.895 | 8.673 | 92.223 | 1.00 | 0.00 | xxxx | 1574 |
| ATOM | 1575 | OE1 | GLU | A | 205 | 56.191 | 8.828 | 91.021 | 1.00 | 0.00 | xxxx | 1575 |
| ATOM | 1576 | OE2 | GLU | A | 205 | 54.874 | 9.172 | 92.719 | 1.00 | 0.00 | xxxx | 1576 |
| ATOM | 1577 | N | VAL | A | 206 | 60.811 | 5.774 | 92.213 | 1.00 | 0.00 | xxxx | 1577 |
| ATOM | 1578 | CA | VAL | A | 206 | 61.734 | 4.913 | 91.459 | 1.00 | 0.00 | xxxx | 1578 |
| ATOM | 1579 | C | VAL | A | 206 | 63.168 | 5.264 | 91.838 | 1.00 | 0.00 | xxxx | 1579 |
| ATOM | 1580 | O | VAL | A | 206 | 63.503 | 6.441 | 91.935 | 1.00 | 0.00 | xxxx | 1580 |
| ATOM | 1581 | CB | VAL | A | 206 | 61.525 | 5.069 | 89.927 | 1.00 | 0.00 | xxxx | 1581 |
| ATOM | 1582 | CG1 | VAL | A | 206 | 62.548 | 4.245 | 89.139 | 1.00 | 0.00 | xxxx | 1582 |
| ATOM | 1583 | CG2 | VAL | A | 206 | 60.096 | 4.661 | 89.499 | 1.00 | 0.00 | xxxx | 1583 |
| ATOM | 1584 | N | VAL | A | 207 | 64.011 | 4.250 | 92.037 | 1.00 | 0.00 | xxxx | 1584 |
| ATOM | 1585 | CA | VAL | A | 207 | 65.445 | 4.454 | 92.239 | 1.00 | 0.00 | xxxx | 1585 |
| ATOM | 1586 | C | VAL | A | 207 | 66.179 | 4.084 | 90.958 | 1.00 | 0.00 | xxxx | 1586 |
| ATOM | 1587 | O | VAL | A | 207 | 66.022 | 2.963 | 90.447 | 1.00 | 0.00 | xxxx | 1587 |
| ATOM | 1588 | CB | VAL | A | 207 | 65.991 | 3.618 | 93.404 | 1.00 | 0.00 | xxxx | 1588 |
| ATOM | 1589 | CG1 | VAL | A | 207 | 67.508 | 3.828 | 93.547 | 1.00 | 0.00 | xxxx | 1589 |
| ATOM | 1590 | CG2 | VAL | A | 207 | 65.272 | 3.946 | 94.703 | 1.00 | 0.00 | xxxx | 1590 |
| ATOM | 1591 | N | ILE | A | 208 | 66.982 | 5.022 | 90.448 | 1.00 | 0.00 | xxxx | 1591 |
| ATOM | 1592 | CA | ILE | A | 208 | 67.799 | 4.793 | 89.254 | 1.00 | 0.00 | xxxx | 1592 |
| ATOM | 1593 | C | ILE | A | 208 | 69.268 | 4.780 | 89.640 | 1.00 | 0.00 | xxxx | 1593 |
| ATOM | 1594 | O | ILE | A | 208 | 69.739 | 5.726 | 90.263 | 1.00 | 0.00 | xxxx | 1594 |
| ATOM | 1595 | CB | ILE | A | 208 | 67.549 | 5.872 | 88.194 | 1.00 | 0.00 | xxxx | 1595 |
| ATOM | 1596 | CG1 | ILE | A | 208 | 66.056 | 5.937 | 87.844 | 1.00 | 0.00 | xxxx | 1596 |
| ATOM | 1597 | CD1 | ILE | A | 208 | 65.698 | 7.103 | 86.935 | 1.00 | 0.00 | xxxx | 1597 |
| ATOM | 1598 | CG2 | ILE | A | 208 | 68.438 | 5.621 | 86.957 | 1.00 | 0.00 | xxxx | 1598 |
| ATOM | 1599 | N | ALA | A | 209 | 69.999 | 3.721 | 89.294 | 1.00 | 0.00 | xxxx | 1599 |
| ATOM | 1600 | CA | ALA | A | 209 | 71.437 | 3.691 | 89.582 | 1.00 | 0.00 | xxxx | 1600 |
| ATOM | 1601 | C | ALA | A | 209 | 72.264 | 3.519 | 88.307 | 1.00 | 0.00 | xxxx | 1601 |
| ATOM | 1602 | O | ALA | A | 209 | 71.869 | 2.778 | 87.405 | 1.00 | 0.00 | xxxx | 1602 |
| ATOM | 1603 | CB | ALA | A | 209 | 71.766 | 2.567 | 90.573 | 1.00 | 0.00 | xxxx | 1603 |
| ATOM | 1604 | N | ASN | A | 210 | 73.415 | 4.187 | 88.240 | 1.00 | 0.00 | xxxx | 1604 |
| ATOM | 1605 | CA | ASN | A | 210 | 74.296 | 4.064 | 87.076 | 1.00 | 0.00 | xxxx | 1605 |
| ATOM | 1606 | C | ASN | A | 210 | 74.921 | 2.682 | 86.965 | 1.00 | 0.00 | xxxx | 1606 |
| ATOM | 1607 | O | ASN | A | 210 | 75.438 | 2.334 | 85.907 | 1.00 | 0.00 | xxxx | 1607 |
| ATOM | 1608 | CB | ASN | A | 210 | 75.461 | 5.062 | 87.115 | 1.00 | 0.00 | xxxx | 1608 |
| ATOM | 1609 | CG | ASN | A | 210 | 75.095 | 6.511 | 86.761 | 1.00 | 0.00 | xxxx | 1609 |
| ATOM | 1610 | OD1 | ASN | A | 210 | 75.978 | 7.361 | 86.862 | 1.00 | 0.00 | xxxx | 1610 |
| ATOM | 1611 | ND2 | ASN | A | 210 | 73.853 | 6.799 | 86.348 | 1.00 | 0.00 | xxxx | 1611 |
| ATOM | 1612 | N | ASN | A | 211 | 74.967 | 1.918 | 88.058 | 1.00 | 0.00 | xxxx | 1612 |
| ATOM | 1613 | CA | ASN | A | 211 | 75.459 | 0.541 | 87.934 | 1.00 | 0.00 | xxxx | 1613 |
| ATOM | 1614 | C | ASN | A | 211 | 74.811 | −0.377 | 88.967 | 1.00 | 0.00 | xxxx | 1614 |
| ATOM | 1615 | O | ASN | A | 211 | 74.096 | 0.077 | 89.878 | 1.00 | 0.00 | xxxx | 1615 |
| ATOM | 1616 | CB | ASN | A | 211 | 77.011 | 0.482 | 87.984 | 1.00 | 0.00 | xxxx | 1616 |
| ATOM | 1617 | CG | ASN | A | 211 | 77.609 | 0.592 | 89.395 | 1.00 | 0.00 | xxxx | 1617 |
| ATOM | 1618 | OD1 | ASN | A | 211 | 76.925 | 0.869 | 90.383 | 1.00 | 0.00 | xxxx | 1618 |
| ATOM | 1619 | ND2 | ASN | A | 211 | 78.929 | 0.407 | 89.473 | 1.00 | 0.00 | xxxx | 1619 |
| ATOM | 1620 | N | ASP | A | 212 | 75.007 | −1.674 | 88.780 | 1.00 | 0.00 | xxxx | 1620 |
| ATOM | 1621 | CA | ASP | A | 212 | 74.353 | −2.659 | 89.644 | 1.00 | 0.00 | xxxx | 1621 |
| ATOM | 1622 | C | ASP | A | 212 | 74.900 | −2.644 | 91.056 | 1.00 | 0.00 | xxxx | 1622 |
| ATOM | 1623 | O | ASP | A | 212 | 74.155 | −2.907 | 91.989 | 1.00 | 0.00 | xxxx | 1623 |
| ATOM | 1624 | CB | ASP | A | 212 | 74.504 | −4.073 | 89.085 | 1.00 | 0.00 | xxxx | 1624 |
| ATOM | 1625 | CG | ASP | A | 212 | 73.580 | −4.354 | 87.923 | 1.00 | 0.00 | xxxx | 1625 |
| ATOM | 1626 | OD1 | ASP | A | 212 | 72.644 | −3.571 | 87.653 | 1.00 | 0.00 | xxxx | 1626 |
| ATOM | 1627 | OD2 | ASP | A | 212 | 73.801 | −5.394 | 87.285 | 1.00 | 0.00 | xxxx | 1627 |
| ATOM | 1628 | N | ALA | A | 213 | 76.201 | −2.392 | 91.216 | 1.00 | 0.00 | xxxx | 1628 |
| ATOM | 1629 | CA | ALA | A | 213 | 76.769 | −2.347 | 92.563 | 1.00 | 0.00 | xxxx | 1629 |
| ATOM | 1630 | C | ALA | A | 213 | 75.986 | −1.340 | 93.403 | 1.00 | 0.00 | xxxx | 1630 |
| ATOM | 1631 | O | ALA | A | 213 | 75.579 | −1.630 | 94.539 | 1.00 | 0.00 | xxxx | 1631 |
| ATOM | 1632 | CB | ALA | A | 213 | 78.250 | −1.988 | 92.519 | 1.00 | 0.00 | xxxx | 1632 |
| ATOM | 1633 | N | MET | A | 214 | 75.725 | −0.174 | 92.827 | 1.00 | 0.00 | xxxx | 1633 |
| ATOM | 1634 | CA | MET | A | 214 | 74.991 | 0.841 | 93.567 | 1.00 | 0.00 | xxxx | 1634 |
| ATOM | 1635 | C | MET | A | 214 | 73.513 | 0.481 | 93.707 | 1.00 | 0.00 | xxxx | 1635 |
| ATOM | 1636 | O | MET | A | 214 | 72.916 | 0.724 | 94.758 | 1.00 | 0.00 | xxxx | 1636 |
| ATOM | 1637 | CB | MET | A | 214 | 75.173 | 2.215 | 92.911 | 1.00 | 0.00 | xxxx | 1637 |
| ATOM | 1638 | CG | MET | A | 214 | 76.604 | 2.734 | 93.068 | 1.00 | 0.00 | xxxx | 1638 |
| ATOM | 1639 | SD | MET | A | 214 | 76.846 | 4.394 | 92.423 | 1.00 | 0.00 | xxxx | 1639 |
| ATOM | 1640 | CE | MET | A | 214 | 76.387 | 4.154 | 90.695 | 1.00 | 0.00 | xxxx | 1640 |
| ATOM | 1641 | N | ALA | A | 215 | 72.917 | −0.091 | 92.665 | 1.00 | 0.00 | xxxx | 1641 |
| ATOM | 1642 | CA | ALA | A | 215 | 71.533 | −0.549 | 92.767 | 1.00 | 0.00 | xxxx | 1642 |
| ATOM | 1643 | C | ALA | A | 215 | 71.363 | −1.525 | 93.930 | 1.00 | 0.00 | xxxx | 1643 |
| ATOM | 1644 | O | ALA | A | 215 | 70.377 | −1.458 | 94.675 | 1.00 | 0.00 | xxxx | 1644 |
| ATOM | 1645 | CB | ALA | A | 215 | 71.087 | −1.196 | 91.455 | 1.00 | 0.00 | xxxx | 1645 |
| ATOM | 1646 | N | MET | A | 216 | 72.321 | −2.433 | 94.098 | 1.00 | 0.00 | xxxx | 1646 |
| ATOM | 1647 | CA | MET | A | 216 | 72.202 | −3.437 | 95.149 | 1.00 | 0.00 | xxxx | 1647 |
| ATOM | 1648 | C | MET | A | 216 | 72.310 | −2.813 | 96.539 | 1.00 | 0.00 | xxxx | 1648 |
| ATOM | 1649 | O | MET | A | 216 | 71.693 | −3.301 | 97.484 | 1.00 | 0.00 | xxxx | 1649 |
| ATOM | 1650 | CB | MET | A | 216 | 73.251 | −4.534 | 94.967 | 1.00 | 0.00 | xxxx | 1650 |
| ATOM | 1651 | CG | MET | A | 216 | 72.994 | −5.385 | 93.737 | 1.00 | 0.00 | xxxx | 1651 |
| ATOM | 1652 | SD | MET | A | 216 | 74.066 | −6.832 | 93.600 | 1.00 | 0.00 | xxxx | 1652 |
| ATOM | 1653 | CE | MET | A | 216 | 75.688 | −6.071 | 93.652 | 1.00 | 0.00 | xxxx | 1653 |
| ATOM | 1654 | N | GLY | A | 217 | 73.077 | −1.733 | 96.661 | 1.00 | 0.00 | xxxx | 1654 |
| ATOM | 1655 | CA | GLY | A | 217 | 73.106 | −0.975 | 97.904 | 1.00 | 0.00 | xxxx | 1655 |
| ATOM | 1656 | C | GLY | A | 217 | 71.764 | −0.328 | 98.196 | 1.00 | 0.00 | xxxx | 1656 |
| ATOM | 1657 | O | GLY | A | 217 | 71.289 | −0.344 | 99.337 | 1.00 | 0.00 | xxxx | 1657 |
| ATOM | 1658 | N | ALA | A | 218 | 71.139 | 0.244 | 97.170 | 1.00 | 0.00 | xxxx | 1658 |
| ATOM | 1659 | CA | ALA | A | 218 | 69.812 | 0.848 | 97.344 | 1.00 | 0.00 | xxxx | 1659 |
| ATOM | 1660 | C | ALA | A | 218 | 68.782 | −0.201 | 97.750 | 1.00 | 0.00 | xxxx | 1660 |
| ATOM | 1661 | O | ALA | A | 218 | 67.937 | 0.070 | 98.601 | 1.00 | 0.00 | xxxx | 1661 |
| ATOM | 1662 | CB | ALA | A | 218 | 69.368 | 1.554 | 96.064 | 1.00 | 0.00 | xxxx | 1662 |
| ATOM | 1663 | N | VAL | A | 219 | 68.859 | −1.393 | 97.149 | 1.00 | 0.00 | xxxx | 1663 |
| ATOM | 1664 | CA | VAL | A | 219 | 67.943 | −2.494 | 97.483 | 1.00 | 0.00 | xxxx | 1664 |
| ATOM | 1665 | C | VAL | A | 219 | 68.054 | −2.817 | 98.970 | 1.00 | 0.00 | xxxx | 1665 |
| ATOM | 1666 | O | VAL | A | 219 | 67.038 | −3.007 | 99.651 | 1.00 | 0.00 | xxxx | 1666 |
| ATOM | 1667 | CB | VAL | A | 219 | 68.231 | −3.743 | 96.618 | 1.00 | 0.00 | xxxx | 1667 |
| ATOM | 1668 | CG1 | VAL | A | 219 | 67.526 | −4.976 | 97.198 | 1.00 | 0.00 | xxxx | 1668 |
| ATOM | 1669 | CG2 | VAL | A | 219 | 67.771 | −3.509 | 95.187 | 1.00 | 0.00 | xxxx | 1669 |
| ATOM | 1670 | N | GLU | A | 220 | 69.283 | −2.856 | 99.481 | 1.00 | 0.00 | xxxx | 1670 |
| ATOM | 1671 | CA | GLU | A | 220 | 69.508 | −3.155 | 100.894 | 1.00 | 0.00 | xxxx | 1671 |
| ATOM | 1672 | C | GLU | A | 220 | 68.901 | −2.089 | 101.804 | 1.00 | 0.00 | xxxx | 1672 |
| ATOM | 1673 | O | GLU | A | 220 | 68.284 | −2.411 | 102.827 | 1.00 | 0.00 | xxxx | 1673 |
| ATOM | 1674 | CB | GLU | A | 220 | 70.998 | −3.275 | 101.196 | 1.00 | 0.00 | xxxx | 1674 |
| ATOM | 1675 | CG | GLU | A | 220 | 71.680 | −4.536 | 100.690 | 1.00 | 0.00 | xxxx | 1675 |
| ATOM | 1676 | CD | GLU | A | 220 | 73.107 | −4.637 | 101.220 | 1.00 | 0.00 | xxxx | 1676 |
| ATOM | 1677 | OE1 | GLU | A | 220 | 73.282 | −4.770 | 102.453 | 1.00 | 0.00 | xxxx | 1677 |
| ATOM | 1678 | OE2 | GLU | A | 220 | 74.055 | −4.552 | 100.413 | 1.00 | 0.00 | xxxx | 1678 |
| ATOM | 1679 | N | ALA | A | 221 | 69.094 | −0.825 | 101.450 | 1.00 | 0.00 | xxxx | 1679 |
| ATOM | 1680 | CA | ALA | A | 221 | 68.532 | 0.265 | 102.247 | 1.00 | 0.00 | xxxx | 1680 |
| ATOM | 1681 | C | ALA | A | 221 | 67.006 | 0.221 | 102.244 | 1.00 | 0.00 | xxxx | 1681 |
| ATOM | 1682 | O | ALA | A | 221 | 66.362 | 0.410 | 103.284 | 1.00 | 0.00 | xxxx | 1682 |
| ATOM | 1683 | CB | ALA | A | 221 | 69.035 | 1.623 | 101.735 | 1.00 | 0.00 | xxxx | 1683 |
| ATOM | 1684 | N | LEU | A | 222 | 66.419 | −0.050 | 101.085 | 1.00 | 0.00 | xxxx | 1684 |
| ATOM | 1685 | CA | LEU | A | 222 | 64.967 | −0.103 | 100.981 | 1.00 | 0.00 | xxxx | 1685 |
| ATOM | 1686 | C | LEU | A | 222 | 64.414 | −1.218 | 101.847 | 1.00 | 0.00 | xxxx | 1686 |
| ATOM | 1687 | O | LEU | A | 222 | 63.394 | −1.040 | 102.523 | 1.00 | 0.00 | xxxx | 1687 |
| ATOM | 1688 | CB | LEU | A | 222 | 64.534 | −0.285 | 99.521 | 1.00 | 0.00 | xxxx | 1688 |
| ATOM | 1689 | CG | LEU | A | 222 | 64.657 | 0.972 | 98.647 | 1.00 | 0.00 | xxxx | 1689 |
| ATOM | 1690 | CD1 | LEU | A | 222 | 64.575 | 0.602 | 97.175 | 1.00 | 0.00 | xxxx | 1690 |
| ATOM | 1691 | CD2 | LEU | A | 222 | 63.573 | 1.992 | 98.994 | 1.00 | 0.00 | xxxx | 1691 |
| ATOM | 1692 | N | LYS | A | 223 | 65.098 | −2.360 | 101.853 | 1.00 | 0.00 | xxxx | 1692 |
| ATOM | 1693 | CA | LYS | A | 223 | 64.672 | −3.492 | 102.687 | 1.00 | 0.00 | xxxx | 1693 |
| ATOM | 1694 | C | LYS | A | 223 | 64.691 | −3.123 | 104.174 | 1.00 | 0.00 | xxxx | 1694 |
| ATOM | 1695 | O | LYS | A | 223 | 63.776 | −3.473 | 104.932 | 1.00 | 0.00 | xxxx | 1695 |
| ATOM | 1696 | CB | LYS | A | 223 | 65.558 | −4.718 | 102.435 | 1.00 | 0.00 | xxxx | 1696 |
| ATOM | 1697 | CG | LYS | A | 223 | 65.379 | −5.837 | 103.462 | 1.00 | 0.00 | xxxx | 1697 |
| ATOM | 1698 | CD | LYS | A | 223 | 66.047 | −7.129 | 103.009 | 1.00 | 0.00 | xxxx | 1698 |
| ATOM | 1699 | CE | LYS | A | 223 | 67.535 | −6.935 | 102.773 | 1.00 | 0.00 | xxxx | 1699 |
| ATOM | 1700 | NZ | LYS | A | 223 | 68.181 | −8.167 | 102.241 | 1.00 | 0.00 | xxxx | 1700 |
| ATOM | 1701 | N | ALA | A | 224 | 65.724 | −2.401 | 104.585 | 1.00 | 0.00 | xxxx | 1701 |
| ATOM | 1702 | CA | ALA | A | 224 | 65.845 | −1.978 | 105.975 | 1.00 | 0.00 | xxxx | 1702 |
| ATOM | 1703 | C | ALA | A | 224 | 64.728 | −1.013 | 106.394 | 1.00 | 0.00 | xxxx | 1703 |
| ATOM | 1704 | O | ALA | A | 224 | 64.380 | −0.950 | 107.574 | 1.00 | 0.00 | xxxx | 1704 |
| ATOM | 1705 | CB | ALA | A | 224 | 67.209 | −1.344 | 106.206 | 1.00 | 0.00 | xxxx | 1705 |
| ATOM | 1706 | N | HIS | A | 225 | 64.162 | −0.281 | 105.437 | 1.00 | 0.00 | xxxx | 1706 |
| ATOM | 1707 | CA | HIS | A | 225 | 63.105 | 0.692 | 105.703 | 1.00 | 0.00 | xxxx | 1707 |
| ATOM | 1708 | C | HIS | A | 225 | 61.712 | 0.158 | 105.372 | 1.00 | 0.00 | xxxx | 1708 |
| ATOM | 1709 | O | HIS | A | 225 | 60.742 | 0.921 | 105.364 | 1.00 | 0.00 | xxxx | 1709 |
| ATOM | 1710 | CB | HIS | A | 225 | 63.340 | 1.974 | 104.904 | 1.00 | 0.00 | xxxx | 1710 |
| ATOM | 1711 | CG | HIS | A | 225 | 64.419 | 2.844 | 105.462 | 1.00 | 0.00 | xxxx | 1711 |
| ATOM | 1712 | ND1 | HIS | A | 225 | 64.152 | 3.941 | 106.253 | 1.00 | 0.00 | xxxx | 1712 |
| ATOM | 1713 | CD2 | HIS | A | 225 | 65.767 | 2.774 | 105.352 | 1.00 | 0.00 | xxxx | 1713 |
| ATOM | 1714 | CE1 | HIS | A | 225 | 65.292 | 4.512 | 106.605 | 1.00 | 0.00 | xxxx | 1714 |
| ATOM | 1715 | NE2 | HIS | A | 225 | 66.287 | 3.823 | 106.074 | 1.00 | 0.00 | xxxx | 1715 |
| ATOM | 1716 | N | ASN | A | 226 | 61.631 | −1.139 | 105.081 | 1.00 | 0.00 | xxxx | 1716 |
| ATOM | 1717 | CA | ASN | A | 226 | 60.369 | −1.793 | 104.741 | 1.00 | 0.00 | xxxx | 1717 |
| ATOM | 1718 | C | ASN | A | 226 | 59.709 | −1.111 | 103.554 | 1.00 | 0.00 | xxxx | 1718 |
| ATOM | 1719 | O | ASN | A | 226 | 58.492 | −0.887 | 103.539 | 1.00 | 0.00 | xxxx | 1719 |
| ATOM | 1720 | CB | ASN | A | 226 | 59.430 | −1.807 | 105.947 | 1.00 | 0.00 | xxxx | 1720 |
| ATOM | 1721 | CG | ASN | A | 226 | 60.045 | −2.499 | 107.142 | 1.00 | 0.00 | xxxx | 1721 |
| ATOM | 1722 | OD1 | ASN | A | 226 | 60.652 | −3.561 | 107.007 | 1.00 | 0.00 | xxxx | 1722 |
| ATOM | 1723 | ND2 | ASN | A | 226 | 59.909 | −1.893 | 108.317 | 1.00 | 0.00 | xxxx | 1723 |
| ATOM | 1724 | N | LYS | A | 227 | 60.537 | −0.777 | 102.565 | 1.00 | 0.00 | xxxx | 1724 |
| ATOM | 1725 | CA | LYS | A | 227 | 60.095 | −0.116 | 101.349 | 1.00 | 0.00 | xxxx | 1725 |
| ATOM | 1726 | C | LYS | A | 227 | 60.554 | −0.907 | 100.120 | 1.00 | 0.00 | xxxx | 1726 |
| ATOM | 1727 | O | LYS | A | 227 | 60.903 | −0.320 | 99.095 | 1.00 | 0.00 | xxxx | 1727 |
| ATOM | 1728 | CB | LYS | A | 227 | 60.642 | 1.319 | 101.285 | 1.00 | 0.00 | xxxx | 1728 |
| ATOM | 1729 | CG | LYS | A | 227 | 60.066 | 2.283 | 102.323 | 1.00 | 0.00 | xxxx | 1729 |
| ATOM | 1730 | CD | LYS | A | 227 | 58.656 | 2.733 | 101.966 | 1.00 | 0.00 | xxxx | 1730 |
| ATOM | 1731 | CE | LYS | A | 227 | 58.656 | 3.636 | 100.738 | 1.00 | 0.00 | xxxx | 1731 |
| ATOM | 1732 | NZ | LYS | A | 227 | 57.319 | 4.258 | 100.483 | 1.00 | 0.00 | xxxx | 1732 |
| ATOM | 1733 | N | SER | A | 228 | 60.548 | −2.232 | 100.216 | 1.00 | 0.00 | xxxx | 1733 |
| ATOM | 1734 | CA | SER | A | 228 | 61.005 | −3.057 | 99.100 | 1.00 | 0.00 | xxxx | 1734 |
| ATOM | 1735 | C | SER | A | 228 | 60.051 | −3.004 | 97.906 | 1.00 | 0.00 | xxxx | 1735 |
| ATOM | 1736 | O | SER | A | 228 | 60.369 | −3.529 | 96.837 | 1.00 | 0.00 | xxxx | 1736 |
| ATOM | 1737 | CB | SER | A | 228 | 61.196 | −4.502 | 99.550 | 1.00 | 0.00 | xxxx | 1737 |
| ATOM | 1738 | OG | SER | A | 228 | 62.308 | −4.609 | 100.420 | 1.00 | 0.00 | xxxx | 1738 |
| ATOM | 1739 | N | SER | A | 229 | 58.902 | −2.360 | 98.089 | 1.00 | 0.00 | xxxx | 1739 |
| ATOM | 1740 | CA | SER | A | 229 | 57.932 | −2.158 | 97.014 | 1.00 | 0.00 | xxxx | 1740 |
| ATOM | 1741 | C | SER | A | 229 | 58.391 | −1.094 | 96.013 | 1.00 | 0.00 | xxxx | 1741 |
| ATOM | 1742 | O | SER | A | 229 | 57.823 | −0.972 | 94.930 | 1.00 | 0.00 | xxxx | 1742 |
| ATOM | 1743 | CB | SER | A | 229 | 56.570 | −1.768 | 97.604 | 1.00 | 0.00 | xxxx | 1743 |
| ATOM | 1744 | OG | SER | A | 229 | 56.710 | −0.696 | 98.528 | 1.00 | 0.00 | xxxx | 1744 |
| ATOM | 1745 | N | ILE | A | 230 | 59.404 | −0.317 | 96.393 | 1.00 | 0.00 | xxxx | 1745 |
| ATOM | 1746 | CA | ILE | A | 230 | 59.980 | 0.706 | 95.516 | 1.00 | 0.00 | xxxx | 1746 |
| ATOM | 1747 | C | ILE | A | 230 | 60.940 | 0.056 | 94.525 | 1.00 | 0.00 | xxxx | 1747 |
| ATOM | 1748 | O | ILE | A | 230 | 61.918 | −0.553 | 94.936 | 1.00 | 0.00 | xxxx | 1748 |
| ATOM | 1749 | CB | ILE | A | 230 | 60.719 | 1.781 | 96.334 | 1.00 | 0.00 | xxxx | 1749 |
| ATOM | 1750 | CG2 | ILE | A | 230 | 61.325 | 2.840 | 95.416 | 1.00 | 0.00 | xxxx | 1750 |
| ATOM | 1751 | CG1 | ILE | A | 230 | 59.795 | 2.397 | 97.395 | 1.00 | 0.00 | xxxx | 1751 |
| ATOM | 1752 | CD1 | ILE | A | 230 | 58.589 | 3.105 | 96.823 | 1.00 | 0.00 | xxxx | 1752 |
| ATOM | 1753 | N | PRO | A | 231 | 60.674 | 0.191 | 93.219 | 1.00 | 0.00 | xxxx | 1753 |
| ATOM | 1754 | CA | PRO | A | 231 | 61.546 | −0.457 | 92.223 | 1.00 | 0.00 | xxxx | 1754 |
| ATOM | 1755 | C | PRO | A | 231 | 62.907 | 0.219 | 92.060 | 1.00 | 0.00 | xxxx | 1755 |
| ATOM | 1756 | O | PRO | A | 231 | 63.019 | 1.446 | 92.161 | 1.00 | 0.00 | xxxx | 1756 |
| ATOM | 1757 | CB | PRO | A | 231 | 60.746 | −0.329 | 90.932 | 1.00 | 0.00 | xxxx | 1757 |
| ATOM | 1758 | CG | PRO | A | 231 | 59.952 | 0.924 | 91.123 | 1.00 | 0.00 | xxxx | 1758 |
| ATOM | 1759 | CD | PRO | A | 231 | 59.570 | 0.934 | 92.587 | 1.00 | 0.00 | xxxx | 1759 |
| ATOM | 1760 | N | VAL | A | 232 | 63.918 | −0.603 | 91.789 | 1.00 | 0.00 | xxxx | 1760 |
| ATOM | 1761 | CA | VAL | A | 232 | 65.289 | −0.161 | 91.571 | 1.00 | 0.00 | xxxx | 1761 |
| ATOM | 1762 | C | VAL | A | 232 | 65.746 | −0.630 | 90.193 | 1.00 | 0.00 | xxxx | 1762 |
| ATOM | 1763 | O | VAL | A | 232 | 65.467 | −1.756 | 89.787 | 1.00 | 0.00 | xxxx | 1763 |
| ATOM | 1764 | CB | VAL | A | 232 | 66.224 | −0.701 | 92.662 | 1.00 | 0.00 | xxxx | 1764 |
| ATOM | 1765 | CG1 | VAL | A | 232 | 67.645 | −0.189 | 92.454 | 1.00 | 0.00 | xxxx | 1765 |
| ATOM | 1766 | CG2 | VAL | A | 232 | 65.710 | −0.305 | 94.046 | 1.00 | 0.00 | xxxx | 1766 |
| ATOM | 1767 | N | PHE | A | 233 | 66.444 | 0.246 | 89.479 | 1.00 | 0.00 | xxxx | 1767 |
| ATOM | 1768 | CA | PHE | A | 233 | 67.018 | −0.105 | 88.177 | 1.00 | 0.00 | xxxx | 1768 |
| ATOM | 1769 | C | PHE | A | 233 | 68.529 | 0.093 | 88.187 | 1.00 | 0.00 | xxxx | 1769 |
| ATOM | 1770 | O | PHE | A | 233 | 69.029 | 1.058 | 88.763 | 1.00 | 0.00 | xxxx | 1770 |
| ATOM | 1771 | CB | PHE | A | 233 | 66.384 | 0.735 | 87.069 | 1.00 | 0.00 | xxxx | 1771 |
| ATOM | 1772 | CG | PHE | A | 233 | 64.923 | 0.445 | 86.854 | 1.00 | 0.00 | xxxx | 1772 |
| ATOM | 1773 | CD1 | PHE | A | 233 | 64.525 | −0.446 | 85.872 | 1.00 | 0.00 | xxxx | 1773 |
| ATOM | 1774 | CD2 | PHE | A | 233 | 63.955 | 1.050 | 87.647 | 1.00 | 0.00 | xxxx | 1774 |
| ATOM | 1775 | CE1 | PHE | A | 233 | 63.175 | −0.719 | 85.664 | 1.00 | 0.00 | xxxx | 1775 |
| ATOM | 1776 | CE2 | PHE | A | 233 | 62.612 | 0.777 | 87.459 | 1.00 | 0.00 | xxxx | 1776 |
| ATOM | 1777 | CZ | PHE | A | 233 | 62.217 | −0.110 | 86.470 | 1.00 | 0.00 | xxxx | 1777 |
| ATOM | 1778 | N | GLY | A | 234 | 69.252 | −0.834 | 87.556 | 1.00 | 0.00 | xxxx | 1778 |
| ATOM | 1779 | CA | GLY | A | 234 | 70.704 | −0.748 | 87.481 | 1.00 | 0.00 | xxxx | 1779 |
| ATOM | 1780 | C | GLY | A | 234 | 71.251 | −0.871 | 86.071 | 1.00 | 0.00 | xxxx | 1780 |
| ATOM | 1781 | O | GLY | A | 234 | 70.506 | −0.818 | 85.087 | 1.00 | 0.00 | xxxx | 1781 |
| ATOM | 1782 | N | VAL | A | 235 | 72.572 | −1.000 | 85.997 | 1.00 | 0.00 | xxxx | 1782 |
| ATOM | 1783 | CA | VAL | A | 235 | 73.304 | −1.326 | 84.767 | 1.00 | 0.00 | xxxx | 1783 |
| ATOM | 1784 | C | VAL | A | 235 | 74.447 | −2.269 | 85.103 | 1.00 | 0.00 | xxxx | 1784 |
| ATOM | 1785 | O | VAL | A | 235 | 75.187 | −2.003 | 86.057 | 1.00 | 0.00 | xxxx | 1785 |
| ATOM | 1786 | CB | VAL | A | 235 | 73.881 | −0.073 | 84.066 | 1.00 | 0.00 | xxxx | 1786 |
| ATOM | 1787 | CG1 | VAL | A | 235 | 74.730 | −0.490 | 82.870 | 1.00 | 0.00 | xxxx | 1787 |
| ATOM | 1788 | CG2 | VAL | A | 235 | 72.785 | 0.883 | 83.642 | 1.00 | 0.00 | xxxx | 1788 |
| ATOM | 1789 | N | ASP | A | 236 | 74.550 | −3.356 | 84.329 | 1.00 | 0.00 | xxxx | 1789 |
| ATOM | 1790 | CA | ASP | A | 236 | 75.711 | −4.274 | 84.192 | 1.00 | 0.00 | xxxx | 1790 |
| ATOM | 1791 | C | ASP | A | 236 | 75.229 | −5.706 | 83.994 | 1.00 | 0.00 | xxxx | 1791 |
| ATOM | 1792 | O | ASP | A | 236 | 75.867 | −6.481 | 83.286 | 1.00 | 0.00 | xxxx | 1792 |
| ATOM | 1793 | CB | ASP | A | 236 | 76.688 | −4.255 | 85.385 | 1.00 | 0.00 | xxxx | 1793 |
| ATOM | 1794 | CG | ASP | A | 236 | 77.665 | −3.089 | 85.333 | 1.00 | 0.00 | xxxx | 1794 |
| ATOM | 1795 | OD1 | ASP | A | 236 | 77.845 | −2.486 | 84.251 | 1.00 | 0.00 | xxxx | 1795 |
| ATOM | 1796 | OD2 | ASP | A | 236 | 78.254 | −2.760 | 86.388 | 1.00 | 0.00 | xxxx | 1796 |
| ATOM | 1797 | N | ALA | A | 237 | 74.107 | −6.042 | 84.628 | 1.00 | 0.00 | xxxx | 1797 |
| ATOM | 1798 | CA | ALA | A | 237 | 73.631 | −7.418 | 84.752 | 1.00 | 0.00 | xxxx | 1798 |
| ATOM | 1799 | C | ALA | A | 237 | 74.721 | −8.308 | 85.339 | 1.00 | 0.00 | xxxx | 1799 |
| ATOM | 1800 | O | ALA | A | 237 | 75.071 | −9.350 | 84.775 | 1.00 | 0.00 | xxxx | 1800 |
| ATOM | 1801 | CB | ALA | A | 237 | 73.144 | −7.963 | 83.398 | 1.00 | 0.00 | xxxx | 1801 |
| ATOM | 1802 | N | LEU | A | 238 | 75.253 | −7.893 | 86.487 | 1.00 | 0.00 | xxxx | 1802 |
| ATOM | 1803 | CA | LEU | A | 238 | 76.190 | −8.722 | 87.231 | 1.00 | 0.00 | xxxx | 1803 |
| ATOM | 1804 | C | LEU | A | 238 | 75.508 | −10.035 | 87.568 | 1.00 | 0.00 | xxxx | 1804 |
| ATOM | 1805 | O | LEU | A | 238 | 74.304 | −10.059 | 87.782 | 1.00 | 0.00 | xxxx | 1805 |
| ATOM | 1806 | CB | LEU | A | 238 | 76.642 | −8.041 | 88.525 | 1.00 | 0.00 | xxxx | 1806 |
| ATOM | 1807 | CG | LEU | A | 238 | 77.270 | −6.648 | 88.486 | 1.00 | 0.00 | xxxx | 1807 |
| ATOM | 1808 | CD1 | LEU | A | 238 | 77.388 | −6.142 | 89.909 | 1.00 | 0.00 | xxxx | 1808 |
| ATOM | 1809 | CD2 | LEU | A | 238 | 78.643 | −6.698 | 87.820 | 1.00 | 0.00 | xxxx | 1809 |
| ATOM | 1810 | N | PRO | A | 239 | 76.280 | −11.130 | 87.626 | 1.00 | 0.00 | xxxx | 1810 |
| ATOM | 1811 | CA | PRO | A | 239 | 75.700 | −12.410 | 88.037 | 1.00 | 0.00 | xxxx | 1811 |
| ATOM | 1812 | C | PRO | A | 239 | 74.853 | −12.285 | 89.303 | 1.00 | 0.00 | xxxx | 1812 |
| ATOM | 1813 | O | PRO | A | 239 | 73.767 | −12.852 | 89.361 | 1.00 | 0.00 | xxxx | 1813 |
| ATOM | 1814 | CB | PRO | A | 239 | 76.932 | −13.286 | 88.276 | 1.00 | 0.00 | xxxx | 1814 |
| ATOM | 1815 | CG | PRO | A | 239 | 77.965 | −12.728 | 87.363 | 1.00 | 0.00 | xxxx | 1815 |
| ATOM | 1816 | CD | PRO | A | 239 | 77.718 | −11.238 | 87.325 | 1.00 | 0.00 | xxxx | 1816 |
| ATOM | 1817 | N | GLU | A | 240 | 75.322 | −11.516 | 90.284 | 1.00 | 0.00 | xxxx | 1817 |
| ATOM | 1818 | CA | GLU | A | 240 | 74.596 | −11.373 | 91.544 | 1.00 | 0.00 | xxxx | 1818 |
| ATOM | 1819 | C | GLU | A | 240 | 73.346 | −10.497 | 91.399 | 1.00 | 0.00 | xxxx | 1819 |
| ATOM | 1820 | O | GLU | A | 240 | 72.385 | −10.645 | 92.152 | 1.00 | 0.00 | xxxx | 1820 |
| ATOM | 1821 | CB | GLU | A | 240 | 75.518 | −10.812 | 92.638 | 1.00 | 0.00 | xxxx | 1821 |
| ATOM | 1822 | CG | GLU | A | 240 | 76.280 | −9.539 | 92.268 | 1.00 | 0.00 | xxxx | 1822 |
| ATOM | 1823 | CD | GLU | A | 240 | 77.688 | −9.807 | 91.744 | 1.00 | 0.00 | xxxx | 1823 |
| ATOM | 1824 | OE1 | GLU | A | 240 | 77.872 | −10.780 | 90.977 | 1.00 | 0.00 | xxxx | 1824 |
| ATOM | 1825 | OE2 | GLU | A | 240 | 78.614 | −9.041 | 92.101 | 1.00 | 0.00 | xxxx | 1825 |
| ATOM | 1826 | N | ALA | A | 241 | 73.349 | −9.599 | 90.421 | 1.00 | 0.00 | xxxx | 1826 |
| ATOM | 1827 | CA | ALA | A | 241 | 72.181 | −8.751 | 90.182 | 1.00 | 0.00 | xxxx | 1827 |
| ATOM | 1828 | C | ALA | A | 241 | 71.053 | −9.528 | 89.496 | 1.00 | 0.00 | xxxx | 1828 |
| ATOM | 1829 | O | ALA | A | 241 | 69.870 | −9.257 | 89.712 | 1.00 | 0.00 | xxxx | 1829 |
| ATOM | 1830 | CB | ALA | A | 241 | 72.572 | −7.540 | 89.350 | 1.00 | 0.00 | xxxx | 1830 |
| ATOM | 1831 | N | LEU | A | 242 | 71.423 | −10.495 | 88.663 | 1.00 | 0.00 | xxxx | 1831 |
| ATOM | 1832 | CA | LEU | A | 242 | 70.431 | −11.298 | 87.963 | 1.00 | 0.00 | xxxx | 1832 |
| ATOM | 1833 | C | LEU | A | 242 | 69.530 | −12.038 | 88.950 | 1.00 | 0.00 | xxxx | 1833 |
| ATOM | 1834 | O | LEU | A | 242 | 68.339 | −12.213 | 88.703 | 1.00 | 0.00 | xxxx | 1834 |
| ATOM | 1835 | CB | LEU | A | 242 | 71.118 | −12.279 | 87.010 | 1.00 | 0.00 | xxxx | 1835 |
| ATOM | 1836 | CG | LEU | A | 242 | 71.898 | −11.594 | 85.884 | 1.00 | 0.00 | xxxx | 1836 |
| ATOM | 1837 | CD1 | LEU | A | 242 | 72.501 | −12.613 | 84.916 | 1.00 | 0.00 | xxxx | 1837 |
| ATOM | 1838 | CD2 | LEU | A | 242 | 71.012 | −10.605 | 85.142 | 1.00 | 0.00 | xxxx | 1838 |
| ATOM | 1839 | N | ALA | A | 243 | 70.095 | −12.448 | 90.081 | 1.00 | 0.00 | xxxx | 1839 |
| ATOM | 1840 | CA | ALA | A | 243 | 69.326 | −13.136 | 91.107 | 1.00 | 0.00 | xxxx | 1840 |
| ATOM | 1841 | C | ALA | A | 243 | 68.253 | −12.225 | 91.705 | 1.00 | 0.00 | xxxx | 1841 |
| ATOM | 1842 | O | ALA | A | 243 | 67.158 | −12.675 | 92.032 | 1.00 | 0.00 | xxxx | 1842 |
| ATOM | 1843 | CB | ALA | A | 243 | 70.249 | −13.654 | 92.196 | 1.00 | 0.00 | xxxx | 1843 |
| ATOM | 1844 | N | LEU | A | 244 | 68.574 | −10.940 | 91.839 | 1.00 | 0.00 | xxxx | 1844 |
| ATOM | 1845 | CA | LEU | A | 244 | 67.627 | −9.962 | 92.351 | 1.00 | 0.00 | xxxx | 1845 |
| ATOM | 1846 | C | LEU | A | 244 | 66.537 | −9.636 | 91.338 | 1.00 | 0.00 | xxxx | 1846 |
| ATOM | 1847 | O | LEU | A | 244 | 65.422 | −9.284 | 91.715 | 1.00 | 0.00 | xxxx | 1847 |
| ATOM | 1848 | CB | LEU | A | 244 | 68.346 | −8.675 | 92.751 | 1.00 | 0.00 | xxxx | 1848 |
| ATOM | 1849 | CG | LEU | A | 244 | 69.396 | −8.786 | 93.856 | 1.00 | 0.00 | xxxx | 1849 |
| ATOM | 1850 | CD1 | LEU | A | 244 | 70.017 | −7.425 | 94.111 | 1.00 | 0.00 | xxxx | 1850 |
| ATOM | 1851 | CD2 | LEU | A | 244 | 68.793 | −9.348 | 95.137 | 1.00 | 0.00 | xxxx | 1851 |
| ATOM | 1852 | N | VAL | A | 245 | 66.867 | −9.713 | 90.050 | 1.00 | 0.00 | xxxx | 1852 |
| ATOM | 1853 | CA | VAL | A | 245 | 65.847 | −9.540 | 89.027 | 1.00 | 0.00 | xxxx | 1853 |
| ATOM | 1854 | C | VAL | A | 245 | 64.835 | −10.677 | 89.141 | 1.00 | 0.00 | xxxx | 1854 |
| ATOM | 1855 | O | VAL | A | 245 | 63.629 | −10.467 | 89.025 | 1.00 | 0.00 | xxxx | 1855 |
| ATOM | 1856 | CB | VAL | A | 245 | 66.460 | −9.483 | 87.612 | 1.00 | 0.00 | xxxx | 1856 |
| ATOM | 1857 | CG1 | VAL | A | 245 | 65.348 | −9.436 | 86.568 | 1.00 | 0.00 | xxxx | 1857 |
| ATOM | 1858 | CG2 | VAL | A | 245 | 67.385 | −8.263 | 87.487 | 1.00 | 0.00 | xxxx | 1858 |
| ATOM | 1859 | N | LYS | A | 246 | 65.342 | −11.882 | 89.377 | 1.00 | 0.00 | xxxx | 1859 |
| ATOM | 1860 | CA | LYS | A | 246 | 64.486 | −13.053 | 89.565 | 1.00 | 0.00 | xxxx | 1860 |
| ATOM | 1861 | C | LYS | A | 246 | 63.547 | −12.843 | 90.752 | 1.00 | 0.00 | xxxx | 1861 |
| ATOM | 1862 | O | LYS | A | 246 | 62.342 | −13.073 | 90.651 | 1.00 | 0.00 | xxxx | 1862 |
| ATOM | 1863 | CB | LYS | A | 246 | 65.339 | −14.313 | 89.764 | 1.00 | 0.00 | xxxx | 1863 |
| ATOM | 1864 | CG | LYS | A | 246 | 64.560 | −15.639 | 89.744 | 1.00 | 0.00 | xxxx | 1864 |
| ATOM | 1865 | CD | LYS | A | 246 | 64.000 | −16.017 | 91.119 | 1.00 | 0.00 | xxxx | 1865 |
| ATOM | 1866 | CE | LYS | A | 246 | 62.993 | −17.166 | 91.028 | 1.00 | 0.00 | xxxx | 1866 |
| ATOM | 1867 | NZ | LYS | A | 246 | 62.123 | −17.249 | 92.240 | 1.00 | 0.00 | xxxx | 1867 |
| ATOM | 1868 | N | SER | A | 247 | 64.105 | −12.396 | 91.874 | 1.00 | 0.00 | xxxx | 1868 |
| ATOM | 1869 | CA | SER | A | 247 | 63.342 | −12.267 | 93.110 | 1.00 | 0.00 | xxxx | 1869 |
| ATOM | 1870 | C | SER | A | 247 | 62.398 | −11.073 | 93.075 | 1.00 | 0.00 | xxxx | 1870 |
| ATOM | 1871 | O | SER | A | 247 | 61.460 | −10.990 | 93.870 | 1.00 | 0.00 | xxxx | 1871 |
| ATOM | 1872 | CB | SER | A | 247 | 64.286 | −12.144 | 94.308 | 1.00 | 0.00 | xxxx | 1872 |
| ATOM | 1873 | OG | SER | A | 247 | 64.953 | −10.894 | 94.307 | 1.00 | 0.00 | xxxx | 1873 |
| ATOM | 1874 | N | GLY | A | 248 | 62.647 | −10.151 | 92.152 | 1.00 | 0.00 | xxxx | 1874 |
| ATOM | 1875 | CA | GLY | A | 248 | 61.848 | −8.948 | 92.052 | 1.00 | 0.00 | xxxx | 1875 |
| ATOM | 1876 | C | GLY | A | 248 | 62.402 | −7.796 | 92.873 | 1.00 | 0.00 | xxxx | 1876 |
| ATOM | 1877 | O | GLY | A | 248 | 61.832 | −6.709 | 92.881 | 1.00 | 0.00 | xxxx | 1877 |
| ATOM | 1878 | N | ALA | A | 249 | 63.517 | −8.035 | 93.561 | 1.00 | 0.00 | xxxx | 1878 |
| ATOM | 1879 | CA | ALA | A | 249 | 64.176 | −6.992 | 94.350 | 1.00 | 0.00 | xxxx | 1879 |
| ATOM | 1880 | C | ALA | A | 249 | 64.775 | −5.920 | 93.447 | 1.00 | 0.00 | xxxx | 1880 |
| ATOM | 1881 | O | ALA | A | 249 | 64.873 | −4.756 | 93.827 | 1.00 | 0.00 | xxxx | 1881 |
| ATOM | 1882 | CB | ALA | A | 249 | 65.253 | −7.598 | 95.238 | 1.00 | 0.00 | xxxx | 1882 |
| ATOM | 1883 | N | LEU | A | 250 | 65.185 | −6.328 | 92.249 | 1.00 | 0.00 | xxxx | 1883 |
| ATOM | 1884 | CA | LEU | A | 250 | 65.663 | −5.410 | 91.230 | 1.00 | 0.00 | xxxx | 1884 |
| ATOM | 1885 | C | LEU | A | 250 | 64.692 | −5.468 | 90.058 | 1.00 | 0.00 | xxxx | 1885 |
| ATOM | 1886 | O | LEU | A | 250 | 64.411 | −6.547 | 89.548 | 1.00 | 0.00 | xxxx | 1886 |
| ATOM | 1887 | CB | LEU | A | 250 | 67.085 | −5.785 | 90.790 | 1.00 | 0.00 | xxxx | 1887 |
| ATOM | 1888 | CG | LEU | A | 250 | 67.898 | −4.821 | 89.938 | 1.00 | 0.00 | xxxx | 1888 |
| ATOM | 1889 | CD1 | LEU | A | 250 | 68.214 | −3.556 | 90.736 | 1.00 | 0.00 | xxxx | 1889 |
| ATOM | 1890 | CD2 | LEU | A | 250 | 69.171 | −5.513 | 89.483 | 1.00 | 0.00 | xxxx | 1890 |
| ATOM | 1891 | N | ALA | A | 251 | 64.174 | −4.319 | 89.633 | 1.00 | 0.00 | xxxx | 1891 |
| ATOM | 1892 | CA | ALA | A | 251 | 63.132 | −4.291 | 88.613 | 1.00 | 0.00 | xxxx | 1892 |
| ATOM | 1893 | C | ALA | A | 251 | 63.704 | −4.469 | 87.215 | 1.00 | 0.00 | xxxx | 1893 |
| ATOM | 1894 | O | ALA | A | 251 | 63.030 | −4.971 | 86.310 | 1.00 | 0.00 | xxxx | 1894 |
| ATOM | 1895 | CB | ALA | A | 251 | 62.332 | −2.985 | 88.696 | 1.00 | 0.00 | xxxx | 1895 |
| ATOM | 1896 | N | GLY | A | 252 | 64.944 | −4.045 | 87.024 | 1.00 | 0.00 | xxxx | 1896 |
| ATOM | 1897 | CA | GLY | A | 252 | 65.556 | −4.215 | 85.724 | 1.00 | 0.00 | xxxx | 1897 |
| ATOM | 1898 | C | GLY | A | 252 | 66.982 | −3.738 | 85.694 | 1.00 | 0.00 | xxxx | 1898 |
| ATOM | 1899 | O | GLY | A | 252 | 67.396 | −2.934 | 86.528 | 1.00 | 0.00 | xxxx | 1899 |
| ATOM | 1900 | N | THR | A | 253 | 67.743 | −4.243 | 84.733 | 1.00 | 0.00 | xxxx | 1900 |
| ATOM | 1901 | CA | THR | A | 253 | 69.114 | −3.796 | 84.577 | 1.00 | 0.00 | xxxx | 1901 |
| ATOM | 1902 | C | THR | A | 253 | 69.496 | −3.891 | 83.101 | 1.00 | 0.00 | xxxx | 1902 |
| ATOM | 1903 | O | THR | A | 253 | 68.648 | −4.124 | 82.251 | 1.00 | 0.00 | xxxx | 1903 |
| ATOM | 1904 | CB | THR | A | 253 | 70.078 | −4.617 | 85.488 | 1.00 | 0.00 | xxxx | 1904 |
| ATOM | 1905 | OG1 | THR | A | 253 | 71.414 | −4.114 | 85.371 | 1.00 | 0.00 | xxxx | 1905 |
| ATOM | 1906 | CG2 | THR | A | 253 | 70.046 | −6.088 | 85.134 | 1.00 | 0.00 | xxxx | 1906 |
| ATOM | 1907 | N | VAL | A | 254 | 70.761 | −3.655 | 82.788 | 1.00 | 0.00 | xxxx | 1907 |
| ATOM | 1908 | CA | VAL | A | 254 | 71.210 | −3.611 | 81.398 | 1.00 | 0.00 | xxxx | 1908 |
| ATOM | 1909 | C | VAL | A | 254 | 72.503 | −4.399 | 81.301 | 1.00 | 0.00 | xxxx | 1909 |
| ATOM | 1910 | O | VAL | A | 254 | 73.468 | −4.053 | 81.986 | 1.00 | 0.00 | xxxx | 1910 |
| ATOM | 1911 | CB | VAL | A | 254 | 71.438 | −2.156 | 80.931 | 1.00 | 0.00 | xxxx | 1911 |
| ATOM | 1912 | CG1 | VAL | A | 254 | 71.966 | −2.120 | 79.492 | 1.00 | 0.00 | xxxx | 1912 |
| ATOM | 1913 | CG2 | VAL | A | 254 | 70.181 | −1.298 | 81.103 | 1.00 | 0.00 | xxxx | 1913 |
| ATOM | 1914 | N | LEU | A | 255 | 72.552 | −5.426 | 80.454 | 1.00 | 0.00 | xxxx | 1914 |
| ATOM | 1915 | CA | LEU | A | 255 | 73.785 | −6.208 | 80.315 | 1.00 | 0.00 | xxxx | 1915 |
| ATOM | 1916 | C | LEU | A | 255 | 74.913 | −5.348 | 79.768 | 1.00 | 0.00 | xxxx | 1916 |
| ATOM | 1917 | O | LEU | A | 255 | 74.817 | −4.771 | 78.689 | 1.00 | 0.00 | xxxx | 1917 |
| ATOM | 1918 | CB | LEU | A | 255 | 73.580 | −7.439 | 79.413 | 1.00 | 0.00 | xxxx | 1918 |
| ATOM | 1919 | CG | LEU | A | 255 | 74.896 | −8.169 | 79.088 | 1.00 | 0.00 | xxxx | 1919 |
| ATOM | 1920 | CD1 | LEU | A | 255 | 75.517 | −8.756 | 80.351 | 1.00 | 0.00 | xxxx | 1920 |
| ATOM | 1921 | CD2 | LEU | A | 255 | 74.714 | −9.252 | 78.028 | 1.00 | 0.00 | xxxx | 1921 |
| ATOM | 1922 | N | ASN | A | 256 | 75.962 | −5.224 | 80.567 | 1.00 | 0.00 | xxxx | 1922 |
| ATOM | 1923 | CA | ASN | A | 256 | 77.230 | −4.676 | 80.127 | 1.00 | 0.00 | xxxx | 1923 |
| ATOM | 1924 | C | ASN | A | 256 | 78.070 | −5.920 | 79.866 | 1.00 | 0.00 | xxxx | 1924 |
| ATOM | 1925 | O | ASN | A | 256 | 78.364 | −6.686 | 80.785 | 1.00 | 0.00 | xxxx | 1925 |
| ATOM | 1926 | CB | ASN | A | 256 | 77.809 | −3.752 | 81.206 | 1.00 | 0.00 | xxxx | 1926 |
| ATOM | 1927 | CG | ASN | A | 256 | 79.029 | −2.990 | 80.750 | 1.00 | 0.00 | xxxx | 1927 |
| ATOM | 1928 | OD1 | ASN | A | 256 | 79.481 | −3.121 | 79.606 | 1.00 | 0.00 | xxxx | 1928 |
| ATOM | 1929 | ND2 | ASN | A | 256 | 79.565 | −2.163 | 81.641 | 1.00 | 0.00 | xxxx | 1929 |
| ATOM | 1930 | N | ASP | A | 257 | 78.380 | −6.168 | 78.598 | 1.00 | 0.00 | xxxx | 1930 |
| ATOM | 1931 | CA | ASP | A | 257 | 78.822 | −7.494 | 78.170 | 1.00 | 0.00 | xxxx | 1931 |
| ATOM | 1932 | C | ASP | A | 257 | 80.300 | −7.722 | 78.470 | 1.00 | 0.00 | xxxx | 1932 |
| ATOM | 1933 | O | ASP | A | 257 | 81.161 | −7.511 | 77.613 | 1.00 | 0.00 | xxxx | 1933 |
| ATOM | 1934 | CB | ASP | A | 257 | 78.534 | −7.665 | 76.672 | 1.00 | 0.00 | xxxx | 1934 |
| ATOM | 1935 | CG | ASP | A | 257 | 78.715 | −9.097 | 76.179 | 1.00 | 0.00 | xxxx | 1935 |
| ATOM | 1936 | OD1 | ASP | A | 257 | 79.319 | −9.930 | 76.887 | 1.00 | 0.00 | xxxx | 1936 |
| ATOM | 1937 | OD2 | ASP | A | 257 | 78.240 | −9.391 | 75.055 | 1.00 | 0.00 | xxxx | 1937 |
| ATOM | 1938 | N | ALA | A | 258 | 80.572 | −8.161 | 79.697 | 1.00 | 0.00 | xxxx | 1938 |
| ATOM | 1939 | CA | ALA | A | 258 | 81.936 | −8.390 | 80.176 | 1.00 | 0.00 | xxxx | 1939 |
| ATOM | 1940 | C | ALA | A | 258 | 82.663 | −9.456 | 79.370 | 1.00 | 0.00 | xxxx | 1940 |
| ATOM | 1941 | O | ALA | A | 258 | 83.844 | −9.286 | 79.049 | 1.00 | 0.00 | xxxx | 1941 |
| ATOM | 1942 | CB | ALA | A | 258 | 81.915 | −8.787 | 81.645 | 1.00 | 0.00 | xxxx | 1942 |
| ATOM | 1943 | N | ASN | A | 259 | 81.969 | −10.547 | 79.044 | 1.00 | 0.00 | xxxx | 1943 |
| ATOM | 1944 | CA | ASN | A | 259 | 82.608 | −11.662 | 78.360 | 1.00 | 0.00 | xxxx | 1944 |
| ATOM | 1945 | C | ASN | A | 259 | 83.116 | −11.236 | 76.988 | 1.00 | 0.00 | xxxx | 1945 |
| ATOM | 1946 | O | ASN | A | 259 | 84.233 | −11.586 | 76.606 | 1.00 | 0.00 | xxxx | 1946 |
| ATOM | 1947 | CB | ASN | A | 259 | 81.649 | −12.850 | 78.230 | 1.00 | 0.00 | xxxx | 1947 |
| ATOM | 1948 | CG | ASN | A | 259 | 81.281 | −13.441 | 79.572 | 1.00 | 0.00 | xxxx | 1948 |
| ATOM | 1949 | OD1 | ASN | A | 259 | 82.086 | −13.437 | 80.505 | 1.00 | 0.00 | xxxx | 1949 |
| ATOM | 1950 | ND2 | ASN | A | 259 | 80.055 | −13.946 | 79.683 | 1.00 | 0.00 | xxxx | 1950 |
| ATOM | 1951 | N | ASN | A | 260 | 82.323 | −10.460 | 76.251 | 1.00 | 0.00 | xxxx | 1951 |
| ATOM | 1952 | CA | ASN | A | 260 | 82.768 | −10.060 | 74.925 | 1.00 | 0.00 | xxxx | 1952 |
| ATOM | 1953 | C | ASN | A | 260 | 83.762 | −8.902 | 74.974 | 1.00 | 0.00 | xxxx | 1953 |
| ATOM | 1954 | O | ASN | A | 260 | 84.652 | −8.842 | 74.128 | 1.00 | 0.00 | xxxx | 1954 |
| ATOM | 1955 | CB | ASN | A | 260 | 81.570 | −9.722 | 74.037 | 1.00 | 0.00 | xxxx | 1955 |
| ATOM | 1956 | CG | ASN | A | 260 | 80.950 | −10.969 | 73.440 | 1.00 | 0.00 | xxxx | 1956 |
| ATOM | 1957 | OD1 | ASN | A | 260 | 81.669 | −11.860 | 72.978 | 1.00 | 0.00 | xxxx | 1957 |
| ATOM | 1958 | ND2 | ASN | A | 260 | 79.627 | −11.059 | 73.469 | 1.00 | 0.00 | xxxx | 1958 |
| ATOM | 1959 | N | GLN | A | 261 | 83.665 | −8.012 | 75.961 | 1.00 | 0.00 | xxxx | 1959 |
| ATOM | 1960 | CA | GLN | A | 261 | 84.693 | −6.975 | 76.100 | 1.00 | 0.00 | xxxx | 1960 |
| ATOM | 1961 | C | GLN | A | 261 | 86.041 | −7.609 | 76.481 | 1.00 | 0.00 | xxxx | 1961 |
| ATOM | 1962 | O | GLN | A | 261 | 87.080 | −7.215 | 75.948 | 1.00 | 0.00 | xxxx | 1962 |
| ATOM | 1963 | CB | GLN | A | 261 | 84.267 | −5.916 | 77.107 | 1.00 | 0.00 | xxxx | 1963 |
| ATOM | 1964 | CG | GLN | A | 261 | 83.183 | −5.025 | 76.535 | 1.00 | 0.00 | xxxx | 1964 |
| ATOM | 1965 | CD | GLN | A | 261 | 82.627 | −4.079 | 77.569 | 1.00 | 0.00 | xxxx | 1965 |
| ATOM | 1966 | OE1 | GLN | A | 261 | 83.322 | −3.172 | 78.048 | 1.00 | 0.00 | xxxx | 1966 |
| ATOM | 1967 | NE2 | GLN | A | 261 | 81.361 | −4.287 | 77.928 | 1.00 | 0.00 | xxxx | 1967 |
| ATOM | 1968 | N | ALA | A | 262 | 86.022 | −8.615 | 77.350 | 1.00 | 0.00 | xxxx | 1968 |
| ATOM | 1969 | CA | ALA | A | 262 | 87.239 | −9.362 | 77.682 | 1.00 | 0.00 | xxxx | 1969 |
| ATOM | 1970 | C | ALA | A | 262 | 87.835 | −10.048 | 76.459 | 1.00 | 0.00 | xxxx | 1970 |
| ATOM | 1971 | O | ALA | A | 262 | 89.052 | −10.010 | 76.237 | 1.00 | 0.00 | xxxx | 1971 |
| ATOM | 1972 | CB | ALA | A | 262 | 86.955 | −10.397 | 78.744 | 1.00 | 0.00 | xxxx | 1972 |
| ATOM | 1973 | N | LYS | A | 263 | 86.970 | −10.684 | 75.672 | 1.00 | 0.00 | xxxx | 1973 |
| ATOM | 1974 | CA | LYS | A | 263 | 87.416 | −11.447 | 74.515 | 1.00 | 0.00 | xxxx | 1974 |
| ATOM | 1975 | C | LYS | A | 263 | 88.009 | −10.539 | 73.440 | 1.00 | 0.00 | xxxx | 1975 |
| ATOM | 1976 | O | LYS | A | 263 | 89.046 | −10.860 | 72.858 | 1.00 | 0.00 | xxxx | 1976 |
| ATOM | 1977 | CB | LYS | A | 263 | 86.259 | −12.264 | 73.932 | 1.00 | 0.00 | xxxx | 1977 |
| ATOM | 1978 | CG | LYS | A | 263 | 86.690 | −13.251 | 72.851 | 1.00 | 0.00 | xxxx | 1978 |
| ATOM | 1979 | CD | LYS | A | 263 | 87.848 | −14.112 | 73.339 | 1.00 | 0.00 | xxxx | 1979 |
| ATOM | 1980 | CE | LYS | A | 263 | 88.295 | −15.120 | 72.290 | 1.00 | 0.00 | xxxx | 1980 |
| ATOM | 1981 | NZ | LYS | A | 263 | 89.591 | −15.754 | 72.673 | 1.00 | 0.00 | xxxx | 1981 |
| ATOM | 1982 | N | ALA | A | 264 | 87.346 | −9.415 | 73.170 | 1.00 | 0.00 | xxxx | 1982 |
| ATOM | 1983 | CA | ALA | A | 264 | 87.865 | −8.451 | 72.197 | 1.00 | 0.00 | xxxx | 1983 |
| ATOM | 1984 | C | ALA | A | 264 | 89.197 | −7.862 | 72.657 | 1.00 | 0.00 | xxxx | 1984 |
| ATOM | 1985 | O | ALA | A | 264 | 90.134 | −7.739 | 71.857 | 1.00 | 0.00 | xxxx | 1985 |
| ATOM | 1986 | CB | ALA | A | 264 | 86.854 | −7.334 | 71.958 | 1.00 | 0.00 | xxxx | 1986 |
| ATOM | 1987 | N | THR | A | 265 | 89.288 | −7.509 | 73.937 | 1.00 | 0.00 | xxxx | 1987 |
| ATOM | 1988 | CA | THR | A | 265 | 90.530 | −6.975 | 74.485 | 1.00 | 0.00 | xxxx | 1988 |
| ATOM | 1989 | C | THR | A | 265 | 91.656 | −7.994 | 74.318 | 1.00 | 0.00 | xxxx | 1989 |
| ATOM | 1990 | O | THR | A | 265 | 92.733 | −7.662 | 73.813 | 1.00 | 0.00 | xxxx | 1990 |
| ATOM | 1991 | CB | THR | A | 265 | 90.350 | −6.589 | 75.958 | 1.00 | 0.00 | xxxx | 1991 |
| ATOM | 1992 | OG1 | THR | A | 265 | 89.369 | −5.554 | 76.042 | 1.00 | 0.00 | xxxx | 1992 |
| ATOM | 1993 | CG2 | THR | A | 265 | 91.662 | −6.075 | 76.577 | 1.00 | 0.00 | xxxx | 1993 |
| ATOM | 1994 | N | PHE | A | 266 | 91.393 | −9.243 | 74.688 | 1.00 | 0.00 | xxxx | 1994 |
| ATOM | 1995 | CA | PHE | A | 266 | 92.401 | −10.285 | 74.533 | 1.00 | 0.00 | xxxx | 1995 |
| ATOM | 1996 | C | PHE | A | 266 | 92.778 | −10.530 | 73.063 | 1.00 | 0.00 | xxxx | 1996 |
| ATOM | 1997 | O | PHE | A | 266 | 93.968 | −10.657 | 72.742 | 1.00 | 0.00 | xxxx | 1997 |
| ATOM | 1998 | CB | PHE | A | 266 | 91.942 | −11.607 | 75.163 | 1.00 | 0.00 | xxxx | 1998 |
| ATOM | 1999 | CG | PHE | A | 266 | 92.949 | −12.702 | 75.008 | 1.00 | 0.00 | xxxx | 1999 |
| ATOM | 2000 | CD1 | PHE | A | 266 | 94.016 | −12.804 | 75.883 | 1.00 | 0.00 | xxxx | 2000 |
| ATOM | 2001 | CD2 | PHE | A | 266 | 92.858 | −13.593 | 73.959 | 1.00 | 0.00 | xxxx | 2001 |
| ATOM | 2002 | CE1 | PHE | A | 266 | 94.970 | −13.800 | 75.724 | 1.00 | 0.00 | xxxx | 2002 |
| ATOM | 2003 | CE2 | PHE | A | 266 | 93.813 | −14.595 | 73.789 | 1.00 | 0.00 | xxxx | 2003 |
| ATOM | 2004 | CZ | PHE | A | 266 | 94.864 | −14.688 | 74.679 | 1.00 | 0.00 | xxxx | 2004 |
| ATOM | 2005 | N | ASP | A | 267 | 91.780 | −10.616 | 72.181 | 1.00 | 0.00 | xxxx | 2005 |
| ATOM | 2006 | CA | ASP | A | 267 | 92.046 | −10.959 | 70.785 | 1.00 | 0.00 | xxxx | 2006 |
| ATOM | 2007 | C | ASP | A | 267 | 92.907 | −9.884 | 70.132 | 1.00 | 0.00 | xxxx | 2007 |
| ATOM | 2008 | O | ASP | A | 267 | 93.848 | −10.181 | 69.389 | 1.00 | 0.00 | xxxx | 2008 |
| ATOM | 2009 | CB | ASP | A | 267 | 90.743 | −11.105 | 69.995 | 1.00 | 0.00 | xxxx | 2009 |
| ATOM | 2010 | CG | ASP | A | 267 | 89.994 | −12.396 | 70.299 | 1.00 | 0.00 | xxxx | 2010 |
| ATOM | 2011 | OD1 | ASP | A | 267 | 90.559 | −13.321 | 70.923 | 1.00 | 0.00 | xxxx | 2011 |
| ATOM | 2012 | OD2 | ASP | A | 267 | 88.826 | −12.487 | 69.876 | 1.00 | 0.00 | xxxx | 2012 |
| ATOM | 2013 | N | LEU | A | 268 | 92.549 | −8.632 | 70.388 | 1.00 | 0.00 | xxxx | 2013 |
| ATOM | 2014 | CA | LEU | A | 268 | 93.291 | −7.518 | 69.797 | 1.00 | 0.00 | xxxx | 2014 |
| ATOM | 2015 | C | LEU | A | 268 | 94.706 | −7.476 | 70.360 | 1.00 | 0.00 | xxxx | 2015 |
| ATOM | 2016 | O | LEU | A | 268 | 95.670 | −7.316 | 69.614 | 1.00 | 0.00 | xxxx | 2016 |
| ATOM | 2017 | CB | LEU | A | 268 | 92.593 | −6.175 | 70.054 | 1.00 | 0.00 | xxxx | 2017 |
| ATOM | 2018 | CG | LEU | A | 268 | 91.587 | −5.752 | 68.976 | 1.00 | 0.00 | xxxx | 2018 |
| ATOM | 2019 | CD1 | LEU | A | 268 | 90.516 | −6.801 | 68.720 | 1.00 | 0.00 | xxxx | 2019 |
| ATOM | 2020 | CD2 | LEU | A | 268 | 90.943 | −4.413 | 69.351 | 1.00 | 0.00 | xxxx | 2020 |
| ATOM | 2021 | N | ALA | A | 269 | 94.841 | −7.635 | 71.673 | 1.00 | 0.00 | xxxx | 2021 |
| ATOM | 2022 | CA | ALA | A | 269 | 96.166 | −7.604 | 72.289 | 1.00 | 0.00 | xxxx | 2022 |
| ATOM | 2023 | C | ALA | A | 269 | 97.052 | −8.722 | 71.752 | 1.00 | 0.00 | xxxx | 2023 |
| ATOM | 2024 | O | ALA | A | 269 | 98.217 | −8.486 | 71.430 | 1.00 | 0.00 | xxxx | 2024 |
| ATOM | 2025 | CB | ALA | A | 269 | 96.046 | −7.696 | 73.795 | 1.00 | 0.00 | xxxx | 2025 |
| ATOM | 2026 | N | LYS | A | 270 | 96.498 | −9.927 | 71.627 | 1.00 | 0.00 | xxxx | 2026 |
| ATOM | 2027 | CA | LYS | A | 270 | 97.277 | −11.069 | 71.131 | 1.00 | 0.00 | xxxx | 2027 |
| ATOM | 2028 | C | LYS | A | 270 | 97.678 | −10.875 | 69.665 | 1.00 | 0.00 | xxxx | 2028 |
| ATOM | 2029 | O | LYS | A | 270 | 98.828 | −11.149 | 69.297 | 1.00 | 0.00 | xxxx | 2029 |
| ATOM | 2030 | CB | LYS | A | 270 | 96.484 | −12.377 | 71.325 | 1.00 | 0.00 | xxxx | 2030 |
| ATOM | 2031 | CG | LYS | A | 270 | 97.152 | −13.678 | 70.800 | 1.00 | 0.00 | xxxx | 2031 |
| ATOM | 2032 | CD | LYS | A | 270 | 98.569 | −13.884 | 71.306 | 1.00 | 0.00 | xxxx | 2032 |
| ATOM | 2033 | CE | LYS | A | 270 | 99.013 | −15.346 | 71.115 | 1.00 | 0.00 | xxxx | 2033 |
| ATOM | 2034 | NZ | LYS | A | 270 | 98.873 | −15.867 | 69.718 | 1.00 | 0.00 | xxxx | 2034 |
| ATOM | 2035 | N | ASN | A | 271 | 96.752 | −10.396 | 68.830 | 1.00 | 0.00 | xxxx | 2035 |
| ATOM | 2036 | CA | ASN | A | 271 | 97.104 | −10.119 | 67.433 | 1.00 | 0.00 | xxxx | 2036 |
| ATOM | 2037 | C | ASN | A | 271 | 98.197 | −9.061 | 67.332 | 1.00 | 0.00 | xxxx | 2037 |
| ATOM | 2038 | O | ASN | A | 271 | 99.155 | −9.216 | 66.560 | 1.00 | 0.00 | xxxx | 2038 |
| ATOM | 2039 | CB | ASN | A | 271 | 95.880 | −9.661 | 66.643 | 1.00 | 0.00 | xxxx | 2039 |
| ATOM | 2040 | CG | ASN | A | 271 | 95.079 | −10.814 | 66.078 | 1.00 | 0.00 | xxxx | 2040 |
| ATOM | 2041 | OD1 | ASN | A | 271 | 95.622 | −11.879 | 65.774 | 1.00 | 0.00 | xxxx | 2041 |
| ATOM | 2042 | ND2 | ASN | A | 271 | 93.778 | −10.592 | 65.903 | 1.00 | 0.00 | xxxx | 2042 |
| ATOM | 2043 | N | LEU | A | 272 | 98.069 | −8.000 | 68.120 | 1.00 | 0.00 | xxxx | 2043 |
| ATOM | 2044 | CA | LEU | A | 272 | 99.074 | −6.935 | 68.080 | 1.00 | 0.00 | xxxx | 2044 |
| ATOM | 2045 | C | LEU | A | 272 | 100.412 | −7.448 | 68.626 | 1.00 | 0.00 | xxxx | 2045 |
| ATOM | 2046 | O | LEU | A | 272 | 101.483 | −7.126 | 68.080 | 1.00 | 0.00 | xxxx | 2046 |
| ATOM | 2047 | CB | LEU | A | 272 | 98.574 | −5.719 | 68.853 | 1.00 | 0.00 | xxxx | 2047 |
| ATOM | 2048 | CG | LEU | A | 272 | 97.416 | −5.022 | 68.138 | 1.00 | 0.00 | xxxx | 2048 |
| ATOM | 2049 | CD1 | LEU | A | 272 | 96.725 | −4.072 | 69.117 | 1.00 | 0.00 | xxxx | 2049 |
| ATOM | 2050 | CD2 | LEU | A | 272 | 97.875 | −4.262 | 66.893 | 1.00 | 0.00 | xxxx | 2050 |
| ATOM | 2051 | N | ALA | A | 273 | 100.373 | −8.263 | 69.681 | 1.00 | 0.00 | xxxx | 2051 |
| ATOM | 2052 | CA | ALA | A | 273 | 101.606 | −8.878 | 70.188 | 1.00 | 0.00 | xxxx | 2052 |
| ATOM | 2053 | C | ALA | A | 273 | 102.310 | −9.679 | 69.093 | 1.00 | 0.00 | xxxx | 2053 |
| ATOM | 2054 | O | ALA | A | 273 | 103.544 | −9.724 | 69.024 | 1.00 | 0.00 | xxxx | 2054 |
| ATOM | 2055 | CB | ALA | A | 273 | 101.302 | −9.785 | 71.385 | 1.00 | 0.00 | xxxx | 2055 |
| ATOM | 2056 | N | ASP | A | 274 | 101.506 | −10.311 | 68.240 | 1.00 | 0.00 | xxxx | 2056 |
| ATOM | 2057 | CA | ASP | A | 274 | 102.014 | −11.193 | 67.195 | 1.00 | 0.00 | xxxx | 2057 |
| ATOM | 2058 | C | ASP | A | 274 | 102.445 | −10.407 | 65.958 | 1.00 | 0.00 | xxxx | 2058 |
| ATOM | 2059 | O | ASP | A | 274 | 102.948 | −10.989 | 64.988 | 1.00 | 0.00 | xxxx | 2059 |
| ATOM | 2060 | CB | ASP | A | 274 | 100.951 | −12.229 | 66.796 | 1.00 | 0.00 | xxxx | 2060 |
| ATOM | 2061 | CG | ASP | A | 274 | 100.725 | −13.303 | 67.854 | 1.00 | 0.00 | xxxx | 2061 |
| ATOM | 2062 | OD1 | ASP | A | 274 | 101.565 | −13.470 | 68.771 | 1.00 | 0.00 | xxxx | 2062 |
| ATOM | 2063 | OD2 | ASP | A | 274 | 99.701 | −14.013 | 67.747 | 1.00 | 0.00 | xxxx | 2063 |
| ATOM | 2064 | N | GLY | A | 275 | 102.244 | −9.094 | 65.978 | 1.00 | 0.00 | xxxx | 2064 |
| ATOM | 2065 | CA | GLY | A | 275 | 102.602 | −8.263 | 64.843 | 1.00 | 0.00 | xxxx | 2065 |
| ATOM | 2066 | C | GLY | A | 275 | 101.597 | −8.298 | 63.706 | 1.00 | 0.00 | xxxx | 2066 |
| ATOM | 2067 | O | GLY | A | 275 | 101.895 | −7.887 | 62.570 | 1.00 | 0.00 | xxxx | 2067 |
| ATOM | 2068 | N | LYS | A | 276 | 100.398 | −8.797 | 64.002 | 1.00 | 0.00 | xxxx | 2068 |
| ATOM | 2069 | CA | LYS | A | 276 | 99.324 | −8.872 | 63.018 | 1.00 | 0.00 | xxxx | 2069 |
| ATOM | 2070 | C | LYS | A | 276 | 98.397 | −7.670 | 63.100 | 1.00 | 0.00 | xxxx | 2070 |
| ATOM | 2071 | O | LYS | A | 276 | 98.509 | −6.852 | 64.015 | 1.00 | 0.00 | xxxx | 2071 |
| ATOM | 2072 | CB | LYS | A | 276 | 98.504 | −10.146 | 63.207 | 1.00 | 0.00 | xxxx | 2072 |
| ATOM | 2073 | CG | LYS | A | 276 | 99.297 | −11.425 | 63.092 | 1.00 | 0.00 | xxxx | 2073 |
| ATOM | 2074 | CD | LYS | A | 276 | 98.417 | −12.580 | 63.509 | 1.00 | 0.00 | xxxx | 2074 |
| ATOM | 2075 | CE | LYS | A | 276 | 99.157 | −13.890 | 63.440 | 1.00 | 0.00 | xxxx | 2075 |
| ATOM | 2076 | NZ | LYS | A | 276 | 98.229 | −15.011 | 63.774 | 1.00 | 0.00 | xxxx | 2076 |
| ATOM | 2077 | N | GLY | A | 277 | 97.474 | −7.581 | 62.143 | 1.00 | 0.00 | xxxx | 2077 |
| ATOM | 2078 | CA | GLY | A | 277 | 96.403 | −6.605 | 62.226 | 1.00 | 0.00 | xxxx | 2078 |
| ATOM | 2079 | C | GLY | A | 277 | 95.544 | −6.908 | 63.435 | 1.00 | 0.00 | xxxx | 2079 |
| ATOM | 2080 | O | GLY | A | 277 | 95.335 | −8.070 | 63.769 | 1.00 | 0.00 | xxxx | 2080 |
| ATOM | 2081 | N | ALA | A | 278 | 95.058 | −5.866 | 64.095 | 1.00 | 0.00 | xxxx | 2081 |
| ATOM | 2082 | CA | ALA | A | 278 | 94.383 | −6.019 | 65.376 | 1.00 | 0.00 | xxxx | 2082 |
| ATOM | 2083 | C | ALA | A | 278 | 93.204 | −6.995 | 65.344 | 1.00 | 0.00 | xxxx | 2083 |
| ATOM | 2084 | O | ALA | A | 278 | 93.006 | −7.747 | 66.303 | 1.00 | 0.00 | xxxx | 2084 |
| ATOM | 2085 | CB | ALA | A | 278 | 93.915 | −4.652 | 65.881 | 1.00 | 0.00 | xxxx | 2085 |
| ATOM | 2086 | N | ALA | A | 279 | 92.432 | −6.986 | 64.256 | 1.00 | 0.00 | xxxx | 2086 |
| ATOM | 2087 | CA | ALA | A | 279 | 91.224 | −7.808 | 64.177 | 1.00 | 0.00 | xxxx | 2087 |
| ATOM | 2088 | C | ALA | A | 279 | 91.413 | −9.072 | 63.342 | 1.00 | 0.00 | xxxx | 2088 |
| ATOM | 2089 | O | ALA | A | 279 | 90.444 | −9.783 | 63.071 | 1.00 | 0.00 | xxxx | 2089 |
| ATOM | 2090 | CB | ALA | A | 279 | 90.071 | −6.989 | 63.612 | 1.00 | 0.00 | xxxx | 2090 |
| ATOM | 2091 | N | ASP | A | 280 | 92.650 | −9.355 | 62.949 | 1.00 | 0.00 | xxxx | 2091 |
| ATOM | 2092 | CA | ASP | A | 280 | 92.942 | −10.490 | 62.077 | 1.00 | 0.00 | xxxx | 2092 |
| ATOM | 2093 | C | ASP | A | 280 | 92.365 | −11.800 | 62.633 | 1.00 | 0.00 | xxxx | 2093 |
| ATOM | 2094 | O | ASP | A | 280 | 92.561 | −12.136 | 63.802 | 1.00 | 0.00 | xxxx | 2094 |
| ATOM | 2095 | CB | ASP | A | 280 | 94.454 | −10.610 | 61.864 | 1.00 | 0.00 | xxxx | 2095 |
| ATOM | 2096 | CG | ASP | A | 280 | 95.004 | −9.556 | 60.903 | 1.00 | 0.00 | xxxx | 2096 |
| ATOM | 2097 | OD1 | ASP | A | 280 | 94.263 | −8.604 | 60.560 | 1.00 | 0.00 | xxxx | 2097 |
| ATOM | 2098 | OD2 | ASP | A | 280 | 96.184 | −9.677 | 60.491 | 1.00 | 0.00 | xxxx | 2098 |
| ATOM | 2099 | N | GLY | A | 281 | 91.611 | −12.511 | 61.802 | 1.00 | 0.00 | xxxx | 2099 |
| ATOM | 2100 | CA | GLY | A | 281 | 91.057 | −13.796 | 62.188 | 1.00 | 0.00 | xxxx | 2100 |
| ATOM | 2101 | C | GLY | A | 281 | 89.859 | −13.718 | 63.117 | 1.00 | 0.00 | xxxx | 2101 |
| ATOM | 2102 | O | GLY | A | 281 | 89.405 | −14.734 | 63.649 | 1.00 | 0.00 | xxxx | 2102 |
| ATOM | 2103 | N | THR | A | 282 | 89.338 | −12.511 | 63.313 | 1.00 | 0.00 | xxxx | 2103 |
| ATOM | 2104 | CA | THR | A | 282 | 88.194 | −12.309 | 64.193 | 1.00 | 0.00 | xxxx | 2104 |
| ATOM | 2105 | C | THR | A | 282 | 87.072 | −11.603 | 63.460 | 1.00 | 0.00 | xxxx | 2105 |
| ATOM | 2106 | O | THR | A | 282 | 87.253 | −11.154 | 62.333 | 1.00 | 0.00 | xxxx | 2106 |
| ATOM | 2107 | CB | THR | A | 282 | 88.542 | −11.459 | 65.418 | 1.00 | 0.00 | xxxx | 2107 |
| ATOM | 2108 | OG1 | THR | A | 282 | 88.572 | −10.079 | 65.029 | 1.00 | 0.00 | xxxx | 2108 |
| ATOM | 2109 | CG2 | THR | A | 282 | 89.899 | −11.850 | 65.993 | 1.00 | 0.00 | xxxx | 2109 |
| ATOM | 2110 | N | ASN | A | 283 | 85.923 | −11.488 | 64.117 | 1.00 | 0.00 | xxxx | 2110 |
| ATOM | 2111 | CA | ASN | A | 283 | 84.809 | −10.731 | 63.558 | 1.00 | 0.00 | xxxx | 2111 |
| ATOM | 2112 | C | ASN | A | 283 | 84.590 | −9.426 | 64.305 | 1.00 | 0.00 | xxxx | 2112 |
| ATOM | 2113 | O | ASN | A | 283 | 83.523 | −8.824 | 64.199 | 1.00 | 0.00 | xxxx | 2113 |
| ATOM | 2114 | CB | ASN | A | 283 | 83.525 | −11.561 | 63.574 | 1.00 | 0.00 | xxxx | 2114 |
| ATOM | 2115 | CG | ASN | A | 283 | 83.481 | −12.583 | 62.453 | 1.00 | 0.00 | xxxx | 2115 |
| ATOM | 2116 | OD1 | ASN | A | 283 | 83.915 | −12.311 | 61.331 | 1.00 | 0.00 | xxxx | 2116 |
| ATOM | 2117 | ND2 | ASN | A | 283 | 82.959 | −13.768 | 62.751 | 1.00 | 0.00 | xxxx | 2117 |
| ATOM | 2118 | N | TRP | A | 284 | 85.595 | −8.977 | 65.059 | 1.00 | 0.00 | xxxx | 2118 |
| ATOM | 2119 | CA | TRP | A | 284 | 85.470 | −7.704 | 65.755 | 1.00 | 0.00 | xxxx | 2119 |
| ATOM | 2120 | C | TRP | A | 284 | 85.444 | −6.558 | 64.757 | 1.00 | 0.00 | xxxx | 2120 |
| ATOM | 2121 | O | TRP | A | 284 | 86.250 | −6.505 | 63.823 | 1.00 | 0.00 | xxxx | 2121 |
| ATOM | 2122 | CB | TRP | A | 284 | 86.608 | −7.493 | 66.762 | 1.00 | 0.00 | xxxx | 2122 |
| ATOM | 2123 | CG | TRP | A | 284 | 86.637 | −8.542 | 67.829 | 1.00 | 0.00 | xxxx | 2123 |
| ATOM | 2124 | CD1 | TRP | A | 284 | 87.626 | −9.461 | 68.055 | 1.00 | 0.00 | xxxx | 2124 |
| ATOM | 2125 | CD2 | TRP | A | 284 | 85.610 | −8.815 | 68.793 | 1.00 | 0.00 | xxxx | 2125 |
| ATOM | 2126 | NE1 | TRP | A | 284 | 87.278 | −10.280 | 69.101 | 1.00 | 0.00 | xxxx | 2126 |
| ATOM | 2127 | CE2 | TRP | A | 284 | 86.047 | −9.900 | 69.574 | 1.00 | 0.00 | xxxx | 2127 |
| ATOM | 2128 | CE3 | TRP | A | 284 | 84.360 | −8.242 | 69.069 | 1.00 | 0.00 | xxxx | 2128 |
| ATOM | 2129 | CZ2 | TRP | A | 284 | 85.283 | −10.432 | 70.607 | 1.00 | 0.00 | xxxx | 2129 |
| ATOM | 2130 | CZ3 | TRP | A | 284 | 83.605 | −8.766 | 70.106 | 1.00 | 0.00 | xxxx | 2130 |
| ATOM | 2131 | CH2 | TRP | A | 284 | 84.069 | −9.846 | 70.861 | 1.00 | 0.00 | xxxx | 2131 |
| ATOM | 2132 | N | LYS | A | 285 | 84.499 | −5.649 | 64.971 | 1.00 | 0.00 | xxxx | 2132 |
| ATOM | 2133 | CA | LYS | A | 285 | 84.365 | −4.451 | 64.160 | 1.00 | 0.00 | xxxx | 2133 |
| ATOM | 2134 | C | LYS | A | 285 | 85.161 | −3.326 | 64.800 | 1.00 | 0.00 | xxxx | 2134 |
| ATOM | 2135 | O | LYS | A | 285 | 84.763 | −2.787 | 65.829 | 1.00 | 0.00 | xxxx | 2135 |
| ATOM | 2136 | CB | LYS | A | 285 | 82.895 | −4.057 | 64.030 | 1.00 | 0.00 | xxxx | 2136 |
| ATOM | 2137 | CG | LYS | A | 285 | 82.649 | −2.809 | 63.208 | 1.00 | 0.00 | xxxx | 2137 |
| ATOM | 2138 | CD | LYS | A | 285 | 81.294 | −2.189 | 63.538 | 1.00 | 0.00 | xxxx | 2138 |
| ATOM | 2139 | CE | LYS | A | 285 | 80.195 | −3.244 | 63.631 | 1.00 | 0.00 | xxxx | 2139 |
| ATOM | 2140 | NZ | LYS | A | 285 | 80.057 | −4.036 | 62.376 | 1.00 | 0.00 | xxxx | 2140 |
| ATOM | 2141 | N | ILE | A | 286 | 86.301 | −2.995 | 64.206 | 1.00 | 0.00 | xxxx | 2141 |
| ATOM | 2142 | CA | ILE | A | 286 | 87.124 | −1.913 | 64.718 | 1.00 | 0.00 | xxxx | 2142 |
| ATOM | 2143 | C | ILE | A | 286 | 86.816 | −0.677 | 63.901 | 1.00 | 0.00 | xxxx | 2143 |
| ATOM | 2144 | O | ILE | A | 286 | 86.990 | −0.675 | 62.677 | 1.00 | 0.00 | xxxx | 2144 |
| ATOM | 2145 | CB | ILE | A | 286 | 88.618 | −2.254 | 64.662 | 1.00 | 0.00 | xxxx | 2145 |
| ATOM | 2146 | CGI | ILE | A | 286 | 88.915 | −3.557 | 65.408 | 1.00 | 0.00 | xxxx | 2146 |
| ATOM | 2147 | CG2 | ILE | A | 286 | 89.437 | −1.152 | 65.286 | 1.00 | 0.00 | xxxx | 2147 |
| ATOM | 2148 | CD1 | ILE | A | 286 | 90.402 | −3.902 | 65.442 | 1.00 | 0.00 | xxxx | 2148 |
| ATOM | 2149 | N | ASP | A | 287 | 86.329 | 0.363 | 64.570 | 1.00 | 0.00 | xxxx | 2149 |
| ATOM | 2150 | CA | ASP | A | 287 | 86.022 | 1.631 | 63.916 | 1.00 | 0.00 | xxxx | 2150 |
| ATOM | 2151 | C | ASP | A | 287 | 86.955 | 2.709 | 64.451 | 1.00 | 0.00 | xxxx | 2151 |
| ATOM | 2152 | O | ASP | A | 287 | 86.837 | 3.105 | 65.616 | 1.00 | 0.00 | xxxx | 2152 |
| ATOM | 2153 | CB | ASP | A | 287 | 84.563 | 2.017 | 64.150 | 1.00 | 0.00 | xxxx | 2153 |
| ATOM | 2154 | CG | ASP | A | 287 | 84.222 | 3.391 | 63.604 | 1.00 | 0.00 | xxxx | 2154 |
| ATOM | 2155 | OD1 | ASP | A | 287 | 83.193 | 3.952 | 64.034 | 1.00 | 0.00 | xxxx | 2155 |
| ATOM | 2156 | OD2 | ASP | A | 287 | 84.976 | 3.911 | 62.754 | 1.00 | 0.00 | xxxx | 2156 |
| ATOM | 2157 | N | ASN | A | 288 | 87.874 | 3.167 | 63.603 | 1.00 | 0.00 | xxxx | 2157 |
| ATOM | 2158 | CA | ASN | A | 288 | 88.884 | 4.149 | 63.997 | 1.00 | 0.00 | xxxx | 2158 |
| ATOM | 2159 | C | ASN | A | 288 | 89.522 | 3.727 | 65.319 | 1.00 | 0.00 | xxxx | 2159 |
| ATOM | 2160 | O | ASN | A | 288 | 89.561 | 4.490 | 66.282 | 1.00 | 0.00 | xxxx | 2160 |
| ATOM | 2161 | CB | ASN | A | 288 | 88.279 | 5.547 | 64.118 | 1.00 | 0.00 | xxxx | 2161 |
| ATOM | 2162 | CG | ASN | A | 288 | 89.326 | 6.645 | 64.007 | 1.00 | 0.00 | xxxx | 2162 |
| ATOM | 2163 | OD1 | ASN | A | 288 | 90.478 | 6.391 | 63.649 | 1.00 | 0.00 | xxxx | 2163 |
| ATOM | 2164 | ND2 | ASN | A | 288 | 88.926 | 7.874 | 64.312 | 1.00 | 0.00 | xxxx | 2164 |
| ATOM | 2165 | N | LYS | A | 289 | 89.969 | 2.475 | 65.337 | 1.00 | 0.00 | xxxx | 2165 |
| ATOM | 2166 | CA | LYS | A | 289 | 90.722 | 1.852 | 66.426 | 1.00 | 0.00 | xxxx | 2166 |
| ATOM | 2167 | C | LYS | A | 289 | 89.913 | 1.565 | 67.685 | 1.00 | 0.00 | xxxx | 2167 |
| ATOM | 2168 | O | LYS | A | 289 | 90.494 | 1.252 | 68.706 | 1.00 | 0.00 | xxxx | 2168 |
| ATOM | 2169 | CB | LYS | A | 289 | 91.943 | 2.703 | 66.761 | 1.00 | 0.00 | xxxx | 2169 |
| ATOM | 2170 | CG | LYS | A | 289 | 92.803 | 2.945 | 65.519 | 1.00 | 0.00 | xxxx | 2170 |
| ATOM | 2171 | CD | LYS | A | 289 | 94.224 | 3.241 | 65.877 | 1.00 | 0.00 | xxxx | 2171 |
| ATOM | 2172 | CE | LYS | A | 289 | 94.317 | 4.511 | 66.697 | 1.00 | 0.00 | xxxx | 2172 |
| ATOM | 2173 | NZ | LYS | A | 289 | 95.738 | 4.684 | 67.101 | 1.00 | 0.00 | xxxx | 2173 |
| ATOM | 2174 | N | VAL | A | 290 | 88.585 | 1.619 | 67.581 | 1.00 | 0.00 | xxxx | 2174 |
| ATOM | 2175 | CA | VAL | A | 290 | 87.691 | 1.387 | 68.712 | 1.00 | 0.00 | xxxx | 2175 |
| ATOM | 2176 | C | VAL | A | 290 | 86.720 | 0.237 | 68.435 | 1.00 | 0.00 | xxxx | 2176 |
| ATOM | 2177 | O | VAL | A | 290 | 86.116 | 0.171 | 67.356 | 1.00 | 0.00 | xxxx | 2177 |
| ATOM | 2178 | CB | VAL | A | 290 | 86.903 | 2.664 | 69.053 | 1.00 | 0.00 | xxxx | 2178 |
| ATOM | 2179 | CG1 | VAL | A | 290 | 85.925 | 2.394 | 70.204 | 1.00 | 0.00 | xxxx | 2179 |
| ATOM | 2180 | CG2 | VAL | A | 290 | 87.847 | 3.824 | 69.381 | 1.00 | 0.00 | xxxx | 2180 |
| ATOM | 2181 | N | VAL | A | 291 | 86.578 | −0.658 | 69.414 | 1.00 | 0.00 | xxxx | 2181 |
| ATOM | 2182 | CA | VAL | A | 291 | 85.515 | −1.666 | 69.437 | 1.00 | 0.00 | xxxx | 2182 |
| ATOM | 2183 | C | VAL | A | 291 | 84.489 | −1.292 | 70.498 | 1.00 | 0.00 | xxxx | 2183 |
| ATOM | 2184 | O | VAL | A | 291 | 84.852 | −1.110 | 71.657 | 1.00 | 0.00 | xxxx | 2184 |
| ATOM | 2185 | CB | VAL | A | 291 | 86.066 | −3.066 | 69.726 | 1.00 | 0.00 | xxxx | 2185 |
| ATOM | 2186 | CG1 | VAL | A | 291 | 84.922 | −4.078 | 69.847 | 1.00 | 0.00 | xxxx | 2186 |
| ATOM | 2187 | CG2 | VAL | A | 291 | 87.077 | −3.484 | 68.661 | 1.00 | 0.00 | xxxx | 2187 |
| ATOM | 2188 | N | ARG | A | 292 | 83.218 | −1.198 | 70.124 | 1.00 | 0.00 | xxxx | 2188 |
| ATOM | 2189 | CA | ARG | A | 292 | 82.153 | −0.990 | 71.106 | 1.00 | 0.00 | xxxx | 2189 |
| ATOM | 2190 | C | ARG | A | 292 | 81.193 | −2.177 | 71.103 | 1.00 | 0.00 | xxxx | 2190 |
| ATOM | 2191 | O | ARG | A | 292 | 80.611 | −2.524 | 70.072 | 1.00 | 0.00 | xxxx | 2191 |
| ATOM | 2192 | CB | ARG | A | 292 | 81.410 | 0.318 | 70.830 | 1.00 | 0.00 | xxxx | 2192 |
| ATOM | 2193 | CG | ARG | A | 292 | 82.339 | 1.527 | 70.900 | 1.00 | 0.00 | xxxx | 2193 |
| ATOM | 2194 | CD | ARG | A | 292 | 81.619 | 2.864 | 70.873 | 1.00 | 0.00 | xxxx | 2194 |
| ATOM | 2195 | NE | ARG | A | 292 | 82.583 | 3.942 | 70.642 | 1.00 | 0.00 | xxxx | 2195 |
| ATOM | 2196 | CZ | ARG | A | 292 | 83.265 | 4.572 | 71.597 | 1.00 | 0.00 | xxxx | 2196 |
| ATOM | 2197 | NH1 | ARG | A | 292 | 83.082 | 4.260 | 72.880 | 1.00 | 0.00 | xxxx | 2197 |
| ATOM | 2198 | NH2 | ARG | A | 292 | 84.133 | 5.524 | 71.270 | 1.00 | 0.00 | xxxx | 2198 |
| ATOM | 2199 | N | VAL | A | 293 | 81.035 | −2.795 | 72.270 | 1.00 | 0.00 | xxxx | 2199 |
| ATOM | 2200 | CA | VAL | A | 293 | 80.206 | −3.979 | 72.446 | 1.00 | 0.00 | xxxx | 2200 |
| ATOM | 2201 | C | VAL | A | 293 | 78.823 | −3.541 | 72.921 | 1.00 | 0.00 | xxxx | 2201 |
| ATOM | 2202 | O | VAL | A | 293 | 78.716 | −2.749 | 73.854 | 1.00 | 0.00 | xxxx | 2202 |
| ATOM | 2203 | CB | VAL | A | 293 | 80.868 | −4.953 | 73.451 | 1.00 | 0.00 | xxxx | 2203 |
| ATOM | 2204 | CG1 | VAL | A | 293 | 80.007 | −6.186 | 73.661 | 1.00 | 0.00 | xxxx | 2204 |
| ATOM | 2205 | CG2 | VAL | A | 293 | 82.286 | −5.342 | 72.966 | 1.00 | 0.00 | xxxx | 2205 |
| ATOM | 2206 | N | PRO | A | 294 | 77.763 | −4.028 | 72.264 | 1.00 | 0.00 | xxxx | 2206 |
| ATOM | 2207 | CA | PRO | A | 294 | 76.400 | −3.585 | 72.591 | 1.00 | 0.00 | xxxx | 2207 |
| ATOM | 2208 | C | PRO | A | 294 | 75.942 | −3.913 | 74.009 | 1.00 | 0.00 | xxxx | 2208 |
| ATOM | 2209 | O | PRO | A | 294 | 76.295 | −4.954 | 74.569 | 1.00 | 0.00 | xxxx | 2209 |
| ATOM | 2210 | CB | PRO | A | 294 | 75.530 | −4.347 | 71.592 | 1.00 | 0.00 | xxxx | 2210 |
| ATOM | 2211 | CG | PRO | A | 294 | 76.441 | −4.689 | 70.475 | 1.00 | 0.00 | xxxx | 2211 |
| ATOM | 2212 | CD | PRO | A | 294 | 77.791 | −4.917 | 71.094 | 1.00 | 0.00 | xxxx | 2212 |
| ATOM | 2213 | N | TYR | A | 295 | 75.146 | −3.004 | 74.563 | 1.00 | 0.00 | xxxx | 2213 |
| ATOM | 2214 | CA | TYR | A | 295 | 74.359 | −3.271 | 75.761 | 1.00 | 0.00 | xxxx | 2214 |
| ATOM | 2215 | C | TYR | A | 295 | 73.038 | −3.956 | 75.407 | 1.00 | 0.00 | xxxx | 2215 |
| ATOM | 2216 | O | TYR | A | 295 | 72.542 | −3.815 | 74.291 | 1.00 | 0.00 | xxxx | 2216 |
| ATOM | 2217 | CB | TYR | A | 295 | 74.047 | −1.974 | 76.497 | 1.00 | 0.00 | xxxx | 2217 |
| ATOM | 2218 | CG | TYR | A | 295 | 75.211 | −1.244 | 77.128 | 1.00 | 0.00 | xxxx | 2218 |
| ATOM | 2219 | CD1 | TYR | A | 295 | 75.657 | −1.576 | 78.401 | 1.00 | 0.00 | xxxx | 2219 |
| ATOM | 2220 | CD2 | TYR | A | 295 | 75.805 | −0.160 | 76.490 | 1.00 | 0.00 | xxxx | 2220 |
| ATOM | 2221 | CE1 | TYR | A | 295 | 76.699 | −0.880 | 79.010 | 1.00 | 0.00 | xxxx | 2221 |
| ATOM | 2222 | CE2 | TYR | A | 295 | 76.839 | 0.540 | 77.088 | 1.00 | 0.00 | xxxx | 2222 |
| ATOM | 2223 | CZ | TYR | A | 295 | 77.287 | 0.179 | 78.349 | 1.00 | 0.00 | xxxx | 2223 |
| ATOM | 2224 | OH | TYR | A | 295 | 78.304 | 0.888 | 78.944 | 1.00 | 0.00 | xxxx | 2224 |
| ATOM | 2225 | N | VAL | A | 296 | 72.448 | −4.668 | 76.368 | 1.00 | 0.00 | xxxx | 2225 |
| ATOM | 2226 | CA | VAL | A | 296 | 71.141 | −5.312 | 76.180 | 1.00 | 0.00 | xxxx | 2226 |
| ATOM | 2227 | C | VAL | A | 296 | 70.270 | −5.142 | 77.430 | 1.00 | 0.00 | xxxx | 2227 |
| ATOM | 2228 | O | VAL | A | 296 | 70.722 | −5.438 | 78.526 | 1.00 | 0.00 | xxxx | 2228 |
| ATOM | 2229 | CB | VAL | A | 296 | 71.290 | −6.820 | 75.854 | 1.00 | 0.00 | xxxx | 2229 |
| ATOM | 2230 | CG1 | VAL | A | 296 | 69.929 | −7.427 | 75.553 | 1.00 | 0.00 | xxxx | 2230 |
| ATOM | 2231 | CG2 | VAL | A | 296 | 72.262 | −7.048 | 74.700 | 1.00 | 0.00 | xxxx | 2231 |
| ATOM | 2232 | N | GLY | A | 297 | 69.024 | −4.691 | 77.273 | 1.00 | 0.00 | xxxx | 2232 |
| ATOM | 2233 | CA | GLY | A | 297 | 68.127 | −4.557 | 78.417 | 1.00 | 0.00 | xxxx | 2233 |
| ATOM | 2234 | C | GLY | A | 297 | 67.730 | −5.896 | 79.010 | 1.00 | 0.00 | xxxx | 2234 |
| ATOM | 2235 | O | GLY | A | 297 | 67.462 | −6.840 | 78.271 | 1.00 | 0.00 | xxxx | 2235 |
| ATOM | 2236 | N | VAL | A | 298 | 67.682 | −5.980 | 80.341 | 1.00 | 0.00 | xxxx | 2236 |
| ATOM | 2237 | CA | VAL | A | 298 | 67.368 | −7.232 | 81.032 | 1.00 | 0.00 | xxxx | 2237 |
| ATOM | 2238 | C | VAL | A | 298 | 66.268 | −7.038 | 82.073 | 1.00 | 0.00 | xxxx | 2238 |
| ATOM | 2239 | O | VAL | A | 298 | 66.392 | −6.199 | 82.965 | 1.00 | 0.00 | xxxx | 2239 |
| ATOM | 2240 | CB | VAL | A | 298 | 68.630 | −7.827 | 81.717 | 1.00 | 0.00 | xxxx | 2240 |
| ATOM | 2241 | CG1 | VAL | A | 298 | 68.279 | −9.124 | 82.444 | 1.00 | 0.00 | xxxx | 2241 |
| ATOM | 2242 | CG2 | VAL | A | 298 | 69.751 | −8.070 | 80.706 | 1.00 | 0.00 | xxxx | 2242 |
| ATOM | 2243 | N | ASP | A | 299 | 65.203 | −7.826 | 81.982 | 1.00 | 0.00 | xxxx | 2243 |
| ATOM | 2244 | CA | ASP | A | 299 | 64.227 | −7.885 | 83.067 | 1.00 | 0.00 | xxxx | 2244 |
| ATOM | 2245 | C | ASP | A | 299 | 63.676 | −9.302 | 83.204 | 1.00 | 0.00 | xxxx | 2245 |
| ATOM | 2246 | O | ASP | A | 299 | 64.224 | −10.238 | 82.626 | 1.00 | 0.00 | xxxx | 2246 |
| ATOM | 2247 | CB | ASP | A | 299 | 63.100 | −6.871 | 82.852 | 1.00 | 0.00 | xxxx | 2247 |
| ATOM | 2248 | CG | ASP | A | 299 | 62.370 | −7.052 | 81.526 | 1.00 | 0.00 | xxxx | 2248 |
| ATOM | 2249 | OD1 | ASP | A | 299 | 62.441 | −8.141 | 80.921 | 1.00 | 0.00 | xxxx | 2249 |
| ATOM | 2250 | OD2 | ASP | A | 299 | 61.711 | −6.090 | 81.088 | 1.00 | 0.00 | xxxx | 2250 |
| ATOM | 2251 | N | LYS | A | 300 | 62.601 | −9.459 | 83.969 | 1.00 | 0.00 | xxxx | 2251 |
| ATOM | 2252 | CA | LYS | A | 300 | 62.028 | −10.784 | 84.188 | 1.00 | 0.00 | xxxx | 2252 |
| ATOM | 2253 | C | LYS | A | 300 | 61.629 | −11.480 | 82.887 | 1.00 | 0.00 | xxxx | 2253 |
| ATOM | 2254 | O | LYS | A | 300 | 61.693 | −12.708 | 82.795 | 1.00 | 0.00 | xxxx | 2254 |
| ATOM | 2255 | CB | LYS | A | 300 | 60.817 | −10.692 | 85.117 | 1.00 | 0.00 | xxxx | 2255 |
| ATOM | 2256 | CG | LYS | A | 300 | 61.178 | −10.695 | 86.589 | 1.00 | 0.00 | xxxx | 2256 |
| ATOM | 2257 | CD | LYS | A | 300 | 59.943 | −10.846 | 87.458 | 1.00 | 0.00 | xxxx | 2257 |
| ATOM | 2258 | CE | LYS | A | 300 | 60.317 | −11.060 | 88.913 | 1.00 | 0.00 | xxxx | 2258 |
| ATOM | 2259 | NZ | LYS | A | 300 | 59.113 | −11.141 | 89.790 | 1.00 | 0.00 | xxxx | 2259 |
| ATOM | 2260 | N | ASP | A | 301 | 61.237 | −10.703 | 81.881 | 1.00 | 0.00 | xxxx | 2260 |
| ATOM | 2261 | CA | ASP | A | 301 | 60.716 | −11.282 | 80.637 | 1.00 | 0.00 | xxxx | 2261 |
| ATOM | 2262 | C | ASP | A | 301 | 61.778 | −12.014 | 79.811 | 1.00 | 0.00 | xxxx | 2262 |
| ATOM | 2263 | O | ASP | A | 301 | 61.472 | −13.011 | 79.157 | 1.00 | 0.00 | xxxx | 2263 |
| ATOM | 2264 | CB | ASP | A | 301 | 60.063 | −10.203 | 79.770 | 1.00 | 0.00 | xxxx | 2264 |
| ATOM | 2265 | CG | ASP | A | 301 | 58.783 | −9.660 | 80.375 | 1.00 | 0.00 | xxxx | 2265 |
| ATOM | 2266 | OD1 | ASP | A | 301 | 58.198 | −10.330 | 81.253 | 1.00 | 0.00 | xxxx | 2266 |
| ATOM | 2267 | OD2 | ASP | A | 301 | 58.354 | −8.562 | 79.961 | 1.00 | 0.00 | xxxx | 2267 |
| ATOM | 2268 | N | ASN | A | 302 | 63.016 | −11.527 | 79.825 | 1.00 | 0.00 | xxxx | 2268 |
| ATOM | 2269 | CA | ASN | A | 302 | 64.069 | −12.161 | 79.033 | 1.00 | 0.00 | xxxx | 2269 |
| ATOM | 2270 | C | ASN | A | 302 | 65.214 | −12.678 | 79.899 | 1.00 | 0.00 | xxxx | 2270 |
| ATOM | 2271 | O | ASN | A | 302 | 66.301 | −12.953 | 79.397 | 1.00 | 0.00 | xxxx | 2271 |
| ATOM | 2272 | CB | ASN | A | 302 | 64.607 | −11.195 | 77.961 | 1.00 | 0.00 | xxxx | 2272 |
| ATOM | 2273 | CG | ASN | A | 302 | 65.396 | −10.021 | 78.546 | 1.00 | 0.00 | xxxx | 2273 |
| ATOM | 2274 | OD1 | ASN | A | 302 | 65.258 | −9.690 | 79.720 | 1.00 | 0.00 | xxxx | 2274 |
| ATOM | 2275 | ND2 | ASN | A | 302 | 66.218 | −9.383 | 77.714 | 1.00 | 0.00 | xxxx | 2275 |
| ATOM | 2276 | N | LEU | A | 303 | 64.950 | −12.842 | 81.192 | 1.00 | 0.00 | xxxx | 2276 |
| ATOM | 2277 | CA | LEU | A | 303 | 65.989 | −13.194 | 82.159 | 1.00 | 0.00 | xxxx | 2277 |
| ATOM | 2278 | C | LEU | A | 303 | 66.640 | −14.545 | 81.863 | 1.00 | 0.00 | xxxx | 2278 |
| ATOM | 2279 | O | LEU | A | 303 | 67.844 | −14.722 | 82.055 | 1.00 | 0.00 | xxxx | 2279 |
| ATOM | 2280 | CB | LEU | A | 303 | 65.406 | −13.204 | 83.575 | 1.00 | 0.00 | xxxx | 2280 |
| ATOM | 2281 | CG | LEU | A | 303 | 66.412 | −13.453 | 84.698 | 1.00 | 0.00 | xxxx | 2281 |
| ATOM | 2282 | CD1 | LEU | A | 303 | 67.491 | −12.378 | 84.690 | 1.00 | 0.00 | xxxx | 2282 |
| ATOM | 2283 | CD2 | LEU | A | 303 | 65.705 | −13.529 | 86.046 | 1.00 | 0.00 | xxxx | 2283 |
| ATOM | 2284 | N | ALA | A | 304 | 65.835 | −15.486 | 81.382 | 1.00 | 0.00 | xxxx | 2284 |
| ATOM | 2285 | CA | ALA | A | 304 | 66.295 | −16.847 | 81.119 | 1.00 | 0.00 | xxxx | 2285 |
| ATOM | 2286 | C | ALA | A | 304 | 67.334 | −16.907 | 79.999 | 1.00 | 0.00 | xxxx | 2286 |
| ATOM | 2287 | O | ALA | A | 304 | 68.003 | −17.926 | 79.822 | 1.00 | 0.00 | xxxx | 2287 |
| ATOM | 2288 | CB | ALA | A | 304 | 65.110 | −17.741 | 80.783 | 1.00 | 0.00 | xxxx | 2288 |
| ATOM | 2289 | O | GLU | A | 305 | 70.668 | −15.086 | 77.723 | 1.00 | 0.00 | xxxx | 2289 |
| ATOM | 2290 | N | GLU | A | 305 | 67.465 | −15.817 | 79.248 | 1.00 | 0.00 | xxxx | 2290 |
| ATOM | 2291 | CA | GLU | A | 305 | 68.405 | −15.758 | 78.133 | 1.00 | 0.00 | xxxx | 2291 |
| ATOM | 2292 | C | GLU | A | 305 | 69.808 | −15.338 | 78.567 | 1.00 | 0.00 | xxxx | 2292 |
| ATOM | 2293 | CB | GLU | A | 305 | 67.897 | −14.793 | 77.060 | 1.00 | 0.00 | xxxx | 2293 |
| ATOM | 2294 | CG | GLU | A | 305 | 66.535 | −15.149 | 76.491 | 1.00 | 0.00 | xxxx | 2294 |
| ATOM | 2295 | CD | GLU | A | 305 | 65.997 | −14.081 | 75.556 | 1.00 | 0.00 | xxxx | 2295 |
| ATOM | 2296 | OE1 | GLU | A | 305 | 66.778 | −13.194 | 75.150 | 1.00 | 0.00 | xxxx | 2296 |
| ATOM | 2297 | OE2 | GLU | A | 305 | 64.790 | −14.124 | 75.230 | 1.00 | 0.00 | xxxx | 2297 |
| ATOM | 2298 | N | PHE | A | 306 | 70.042 | −15.263 | 79.874 | 1.00 | 0.00 | xxxx | 2298 |
| ATOM | 2299 | CA | PHE | A | 306 | 71.332 | −14.800 | 80.384 | 1.00 | 0.00 | xxxx | 2299 |
| ATOM | 2300 | C | PHE | A | 306 | 71.923 | −15.759 | 81.414 | 1.00 | 0.00 | xxxx | 2300 |
| ATOM | 2301 | O | PHE | A | 306 | 71.297 | −16.063 | 82.430 | 1.00 | 0.00 | xxxx | 2301 |
| ATOM | 2302 | CB | PHE | A | 306 | 71.190 | −13.397 | 80.983 | 1.00 | 0.00 | xxxx | 2302 |
| ATOM | 2303 | CG | PHE | A | 306 | 70.743 | −12.366 | 79.989 | 1.00 | 0.00 | xxxx | 2303 |
| ATOM | 2304 | CD1 | PHE | A | 306 | 71.669 | −11.643 | 79.258 | 1.00 | 0.00 | xxxx | 2304 |
| ATOM | 2305 | CD2 | PHE | A | 306 | 69.394 | −12.139 | 79.766 | 1.00 | 0.00 | xxxx | 2305 |
| ATOM | 2306 | CE1 | PHE | A | 306 | 71.259 | −10.703 | 78.330 | 1.00 | 0.00 | xxxx | 2306 |
| ATOM | 2307 | CE2 | PHE | A | 306 | 68.979 | −11.206 | 78.839 | 1.00 | 0.00 | xxxx | 2307 |
| ATOM | 2308 | CZ | PHE | A | 306 | 69.912 | −10.488 | 78.118 | 1.00 | 0.00 | xxxx | 2308 |
| ATOM | 2309 | N | SER | A | 307 | 73.135 | −16.234 | 81.137 | 1.00 | 0.00 | xxxx | 2309 |
| ATOM | 2310 | CA | SER | A | 307 | 73.836 | −17.143 | 82.037 | 1.00 | 0.00 | xxxx | 2310 |
| ATOM | 2311 | C | SER | A | 307 | 74.912 | −16.399 | 82.823 | 1.00 | 0.00 | xxxx | 2311 |
| ATOM | 2312 | O | SER | A | 307 | 74.852 | −15.178 | 82.974 | 1.00 | 0.00 | xxxx | 2312 |
| ATOM | 2313 | CB | SER | A | 307 | 74.460 | −18.305 | 81.257 | 1.00 | 0.00 | xxxx | 2313 |
| ATOM | 2314 | OG | SER | A | 307 | 73.468 | −19.075 | 80.596 | 1.00 | 0.00 | xxxx | 2314 |
| HETATM | 2315 | C2 | GLC | B | 1 | 92.690 | −2.746 | 98.842 | 1.00 | 0.00 | xxxx | 2315 |
| HETATM | 2316 | C3 | GLC | B | 1 | 92.583 | −2.324 | 97.414 | 1.00 | 0.00 | xxxx | 2316 |
| HETATM | 2317 | C4 | GLC | B | 1 | 92.596 | −3.496 | 96.471 | 1.00 | 0.00 | xxxx | 2317 |
| HETATM | 2318 | C5 | GLC | B | 1 | 91.481 | −4.463 | 96.869 | 1.00 | 0.00 | xxxx | 2318 |
| HETATM | 2319 | C6 | GLC | B | 1 | 91.486 | −5.686 | 96.009 | 1.00 | 0.00 | xxxx | 2319 |
| HETATM | 2320 | C1 | GLC | B | 1 | 91.608 | −3.754 | 99.186 | 1.00 | 0.00 | xxxx | 2320 |
| HETATM | 2321 | O1 | GLC | B | 1 | 91.764 | −4.278 | 100.438 | 1.00 | 0.00 | xxxx | 2321 |
| HETATM | 2322 | O2 | GLC | B | 1 | 92.488 | −1.602 | 99.689 | 1.00 | 0.00 | xxxx | 2322 |
| HETATM | 2323 | O3 | GLC | B | 1 | 93.663 | −1.404 | 97.079 | 1.00 | 0.00 | xxxx | 2323 |
| HETATM | 2324 | O4 | GLC | B | 1 | 92.389 | −3.037 | 95.130 | 1.00 | 0.00 | xxxx | 2324 |
| HETATM | 2325 | O5 | GLC | B | 1 | 91.680 | −4.901 | 98.250 | 1.00 | 0.00 | xxxx | 2325 |
| HETATM | 2326 | O6 | GLC | B | 1 | 90.408 | −6.542 | 96.329 | 1.00 | 0.00 | xxxx | 2326 |
| HETATM | 2327 | C2 | GLC | B | 1 | 83.021 | −4.575 | 97.570 | 1.00 | 0.00 | xxxx | 2327 |
| HETATM | 2328 | C3 | GLC | B | 1 | 81.832 | −4.206 | 96.753 | 1.00 | 0.00 | xxxx | 2328 |
| HETATM | 2329 | C4 | GLC | B | 1 | 81.920 | −2.801 | 96.215 | 1.00 | 0.00 | xxxx | 2329 |
| HETATM | 2330 | C5 | GLC | B | 1 | 83.229 | −2.630 | 95.437 | 1.00 | 0.00 | xxxx | 2330 |
| HETATM | 2331 | C6 | GLC | B | 1 | 83.321 | −1.238 | 94.879 | 1.00 | 0.00 | xxxx | 2331 |
| HETATM | 2332 | C1 | GLC | B | 1 | 84.339 | −4.267 | 96.878 | 1.00 | 0.00 | xxxx | 2332 |
| HETATM | 2333 | O1 | GLC | B | 1 | 85.348 | −4.414 | 97.783 | 1.00 | 0.00 | xxxx | 2333 |
| HETATM | 2334 | O2 | GLC | B | 1 | 82.974 | −5.984 | 97.880 | 1.00 | 0.00 | xxxx | 2334 |
| HETATM | 2335 | O3 | GLC | B | 1 | 80.691 | −4.345 | 97.633 | 1.00 | 0.00 | xxxx | 2335 |
| HETATM | 2336 | O4 | GLC | B | 1 | 80.814 | −2.458 | 95.361 | 1.00 | 0.00 | xxxx | 2336 |
| HETATM | 2337 | O5 | GLC | B | 1 | 84.362 | −2.867 | 96.341 | 1.00 | 0.00 | xxxx | 2337 |
| HETATM | 2338 | O6 | GLC | B | 1 | 84.340 | −1.167 | 93.916 | 1.00 | 0.00 | xxxx | 2338 |
| HETATM | 2339 | C2 | GLC | B | 1 | 80.567 | −0.156 | 84.804 | 1.00 | 0.00 | xxxx | 2339 |
| HETATM | 2340 | C3 | GLC | B | 1 | 80.797 | 0.238 | 86.214 | 1.00 | 0.00 | xxxx | 2340 |
| HETATM | 2341 | C4 | GLC | B | 1 | 82.268 | 0.274 | 86.520 | 1.00 | 0.00 | xxxx | 2341 |
| HETATM | 2342 | C5 | GLC | B | 1 | 83.019 | 1.200 | 85.560 | 1.00 | 0.00 | xxxx | 2342 |
| HETATM | 2343 | C6 | GLC | B | 1 | 84.494 | 1.071 | 85.797 | 1.00 | 0.00 | xxxx | 2343 |
| HETATM | 2344 | C1 | GLC | B | 1 | 81.296 | 0.784 | 83.863 | 1.00 | 0.00 | xxxx | 2344 |
| HETATM | 2345 | O1 | GLC | B | 1 | 81.119 | 0.356 | 82.586 | 1.00 | 0.00 | xxxx | 2345 |
| HETATM | 2346 | O2 | GLC | B | 1 | 79.162 | −0.097 | 84.487 | 1.00 | 0.00 | xxxx | 2346 |
| HETATM | 2347 | O3 | GLC | B | 1 | 80.162 | −0.738 | 87.080 | 1.00 | 0.00 | xxxx | 2347 |
| HETATM | 2348 | O4 | GLC | B | 1 | 82.448 | 0.724 | 87.865 | 1.00 | 0.00 | xxxx | 2348 |
| HETATM | 2349 | O5 | GLC | B | 1 | 82.760 | 0.835 | 84.165 | 1.00 | 0.00 | xxxx | 2349 |
| HETATM | 2350 | O6 | GLC | B | 1 | 85.215 | 1.890 | 84.906 | 1.00 | 0.00 | xxxx | 2350 |
| HETATM | 2351 | C01 | ACR | H | 1 | 89.320 | 8.265 | 96.523 | 1.00 | 0.00 | xxxx | 2351 |
| HETATM | 2352 | N02 | ACR | H | 1 | 88.307 | 7.240 | 96.350 | 1.00 | 0.00 | xxxx | 2352 |
| HETATM | 2353 | C03 | ACR | H | 1 | 87.743 | 6.573 | 97.511 | 1.00 | 0.00 | xxxx | 2353 |
| HETATM | 2354 | C04 | ACR | H | 1 | 87.854 | 6.877 | 95.019 | 1.00 | 0.00 | xxxx | 2354 |
| HETATM | 2355 | C05 | ACR | H | 1 | 86.887 | 5.890 | 94.867 | 1.00 | 0.00 | xxxx | 2355 |
| HETATM | 2356 | C06 | ACR | H | 1 | 86.444 | 5.530 | 93.561 | 1.00 | 0.00 | xxxx | 2356 |
| HETATM | 2357 | C07 | ACR | H | 1 | 86.959 | 6.149 | 92.460 | 1.00 | 0.00 | xxxx | 2357 |
| HETATM | 2358 | C08 | ACR | H | 1 | 87.951 | 7.162 | 92.617 | 1.00 | 0.00 | xxxx | 2358 |
| HETATM | 2359 | C09 | ACR | H | 1 | 88.389 | 7.511 | 93.893 | 1.00 | 0.00 | xxxx | 2359 |
| HETATM | 2360 | C10 | ACR | H | 1 | 86.503 | 5.785 | 91.156 | 1.00 | 0.00 | xxxx | 2360 |
| HETATM | 2361 | C11 | ACR | H | 1 | 85.537 | 4.795 | 91.016 | 1.00 | 0.00 | xxxx | 2361 |
| HETATM | 2362 | C12 | ACR | H | 1 | 85.018 | 4.165 | 92.145 | 1.00 | 0.00 | xxxx | 2362 |
| HETATM | 2363 | C13 | ACR | H | 1 | 85.455 | 4.521 | 93.413 | 1.00 | 0.00 | xxxx | 2363 |
| HETATM | 2364 | C14 | ACR | H | 1 | 85.016 | 4.366 | 89.647 | 1.00 | 0.00 | xxxx | 2364 |
| HETATM | 2365 | O15 | ACR | H | 1 | 85.422 | 4.856 | 88.645 | 1.00 | 0.00 | xxxx | 2365 |
| HETATM | 2366 | C16 | ACR | H | 1 | 83.949 | 3.277 | 89.605 | 1.00 | 0.00 | xxxx | 2366 |
| HETATM | 2367 | C17 | ACR | H | 1 | 82.796 | 3.601 | 88.672 | 1.00 | 0.00 | xxxx | 2367 |
| HETATM | 2368 | C2 | GLC | E | 1 | 74.043 | 3.534 | 106.784 | 1.00 | 0.00 | xxxx | 2368 |
| HETATM | 2369 | C3 | GLC | E | 1 | 73.870 | 4.007 | 108.173 | 1.00 | 0.00 | xxxx | 2369 |
| HETATM | 2370 | C4 | GLC | E | 1 | 72.769 | 3.314 | 108.911 | 1.00 | 0.00 | xxxx | 2370 |
| HETATM | 2371 | C5 | GLC | E | 1 | 72.652 | 1.800 | 108.679 | 1.00 | 0.00 | xxxx | 2371 |
| HETATM | 2372 | C6 | GLC | E | 1 | 71.229 | 1.409 | 108.957 | 1.00 | 0.00 | xxxx | 2372 |
| HETATM | 2373 | C1 | GLC | E | 1 | 74.196 | 2.025 | 106.743 | 1.00 | 0.00 | xxxx | 2373 |
| HETATM | 2374 | O1 | GLC | E | 1 | 74.348 | 1.636 | 105.448 | 1.00 | 0.00 | xxxx | 2374 |
| HETATM | 2375 | O2 | GLC | E | 1 | 75.235 | 4.143 | 106.253 | 1.00 | 0.00 | xxxx | 2375 |
| HETATM | 2376 | O3 | GLC | E | 1 | 73.539 | 5.421 | 108.139 | 1.00 | 0.00 | xxxx | 2376 |
| HETATM | 2377 | O4 | GLC | E | 1 | 73.014 | 3.548 | 110.299 | 1.00 | 0.00 | xxxx | 2377 |
| HETATM | 2378 | O5 | GLC | E | 1 | 72.987 | 1.347 | 107.318 | 1.00 | 0.00 | xxxx | 2378 |
| HETATM | 2379 | O6 | GLC | E | 1 | 70.837 | 0.351 | 108.121 | 1.00 | 0.00 | xxxx | 2379 |
| HETATM | 2380 | C2 | GLC | E | 1 | 63.097 | −3.500 | 75.382 | 1.00 | 0.00 | xxxx | 2380 |
| HETATM | 2381 | C3 | GLC | E | 1 | 64.434 | −4.145 | 75.413 | 1.00 | 0.00 | xxxx | 2381 |
| HETATM | 2382 | C4 | GLC | E | 1 | 64.610 | −5.099 | 76.560 | 1.00 | 0.00 | xxxx | 2382 |
| HETATM | 2383 | C5 | GLC | E | 1 | 63.418 | −6.043 | 76.763 | 1.00 | 0.00 | xxxx | 2383 |
| HETATM | 2384 | C6 | GLC | E | 1 | 63.551 | −6.742 | 78.085 | 1.00 | 0.00 | xxxx | 2384 |
| HETATM | 2385 | C1 | GLC | E | 1 | 61.987 | −4.531 | 75.469 | 1.00 | 0.00 | xxxx | 2385 |
| HETATM | 2386 | O1 | GLC | E | 1 | 60.787 | −3.891 | 75.460 | 1.00 | 0.00 | xxxx | 2386 |
| HETATM | 2387 | O2 | GLC | E | 1 | 62.942 | −2.769 | 74.151 | 1.00 | 0.00 | xxxx | 2387 |
| HETATM | 2388 | O3 | GLC | E | 1 | 65.458 | −3.110 | 75.509 | 1.00 | 0.00 | xxxx | 2388 |
| HETATM | 2389 | O4 | GLC | E | 1 | 65.777 | −5.887 | 76.302 | 1.00 | 0.00 | xxxx | 2389 |
| HETATM | 2390 | O5 | GLC | E | 1 | 62.124 | −5.342 | 76.723 | 1.00 | 0.00 | xxxx | 2390 |
| HETATM | 2391 | O6 | GLC | E | 1 | 62.509 | −7.677 | 78.244 | 1.00 | 0.00 | xxxx | 2391 |
| HETATM | 2392 | CA | CA | M | 1 | 54.443 | 10.528 | 90.671 | 1.00 | 0.00 | xxxx | 2392 |
| HETATM | 2393 | O | HOH | S | 1 | 64.967 | 12.689 | 101.419 | 1.00 | 0.00 | xxxx | 2393 |
| HETATM | 2394 | O | HOH | S | 2 | 71.563 | 5.785 | 84.976 | 1.00 | 0.00 | xxxx | 2394 |
| HETATM | 2395 | O | HOH | S | 3 | 80.923 | −2.340 | 89.295 | 1.00 | 0.00 | xxxx | 2395 |
| HETATM | 2396 | O | HOH | S | 4 | 56.757 | 3.515 | 93.548 | 1.00 | 0.00 | xxxx | 2396 |
| HETATM | 2397 | O | HOH | S | 5 | 95.702 | −5.521 | 95.382 | 1.00 | 0.00 | xxxx | 2397 |
| HETATM | 2398 | O | HOH | S | 6 | 78.131 | −2.907 | 89.098 | 1.00 | 0.00 | xxxx | 2398 |
| HETATM | 2399 | O | HOH | S | 7 | 78.263 | −4.245 | 76.555 | 1.00 | 0.00 | xxxx | 2399 |
| HETATM | 2400 | O | HOH | S | 8 | 93.988 | −3.398 | 102.054 | 1.00 | 0.00 | xxxx | 2400 |
| HETATM | 2401 | O | HOH | S | 9 | 83.364 | 2.281 | 77.076 | 1.00 | 0.00 | xxxx | 2401 |
| HETATM | 2402 | O | HOH | S | 10 | 55.561 | 10.622 | 95.053 | 1.00 | 0.00 | xxxx | 2402 |
| HETATM | 2403 | O | HOH | S | 11 | 56.036 | 1.251 | 94.793 | 1.00 | 0.00 | xxxx | 2403 |
| HETATM | 2404 | O | HOH | S | 12 | 92.822 | 10.636 | 77.633 | 1.00 | 0.00 | xxxx | 2404 |
| HETATM | 2405 | O | HOH | S | 13 | 103.721 | −4.910 | 86.930 | 1.00 | 0.00 | xxxx | 2405 |
| HETATM | 2406 | O | HOH | S | 14 | 84.655 | 4.673 | 79.569 | 1.00 | 0.00 | xxxx | 2406 |
| HETATM | 2407 | O | HOH | S | 15 | 86.063 | −2.878 | 78.146 | 1.00 | 0.00 | xxxx | 2407 |
| HETATM | 2408 | O | HOH | S | 16 | 63.999 | 15.452 | 85.780 | 1.00 | 0.00 | xxxx | 2408 |
| HETATM | 2409 | O | HOH | S | 17 | 97.891 | −19.300 | 78.614 | 1.00 | 0.00 | xxxx | 2409 |
| HETATM | 2410 | O | HOH | S | 18 | 60.292 | 16.203 | 83.158 | 1.00 | 0.00 | xxxx | 2410 |
| HETATM | 2411 | O | HOH | S | 19 | 83.019 | 4.976 | 77.084 | 1.00 | 0.00 | xxxx | 2411 |
| HETATM | 2412 | O | HOH | S | 20 | 78.782 | −5.387 | 95.995 | 1.00 | 0.00 | xxxx | 2412 |
| HETATM | 2413 | O | HOH | S | 21 | 82.607 | −1.435 | 80.011 | 1.00 | 0.00 | xxxx | 2413 |
| HETATM | 2414 | O | HOH | S | 22 | 63.199 | 16.555 | 83.175 | 1.00 | 0.00 | xxxx | 2414 |
| HETATM | 2415 | O | HOH | S | 23 | 97.299 | −13.937 | 66.671 | 1.00 | 0.00 | xxxx | 2415 |
| HETATM | 2416 | O | HOH | S | 24 | 77.647 | −0.308 | 73.329 | 1.00 | 0.00 | xxxx | 2416 |
| HETATM | 2417 | O | HOH | S | 25 | 72.956 | 6.465 | 105.943 | 1.00 | 0.00 | xxxx | 2417 |
| HETATM | 2418 | O | HOH | S | 26 | 54.476 | 12.405 | 82.647 | 1.00 | 0.00 | xxxx | 2418 |
| HETATM | 2419 | O | HOH | S | 27 | 103.889 | −13.594 | 65.061 | 1.00 | 0.00 | xxxx | 2419 |
| HETATM | 2420 | O | HOH | S | 28 | 69.798 | 6.225 | 109.324 | 1.00 | 0.00 | xxxx | 2420 |
| HETATM | 2421 | O | HOH | S | 29 | 82.160 | −1.182 | 91.535 | 1.00 | 0.00 | xxxx | 2421 |
| HETATM | 2422 | O | HOH | S | 30 | 95.116 | −13.976 | 64.095 | 1.00 | 0.00 | xxxx | 2422 |
| HETATM | 2423 | O | HOH | S | 31 | 63.291 | −3.325 | 92.624 | 1.00 | 0.00 | xxxx | 2423 |
| HETATM | 2424 | O | HOH | S | 32 | 51.558 | 16.241 | 86.760 | 1.00 | 0.00 | xxxx | 2424 |
| HETATM | 2425 | O | HOH | S | 33 | 82.692 | 1.250 | 79.488 | 1.00 | 0.00 | xxxx | 2425 |
| HETATM | 2426 | O | HOH | S | 34 | 58.233 | 14.653 | 79.027 | 1.00 | 0.00 | xxxx | 2426 |
| HETATM | 2427 | O | HOH | S | 35 | 76.576 | −7.589 | 73.550 | 1.00 | 0.00 | xxxx | 2427 |
| HETATM | 2428 | O | HOH | S | 36 | 101.994 | −4.603 | 91.215 | 1.00 | 0.00 | xxxx | 2428 |
| HETATM | 2429 | O | HOH | S | 37 | 52.930 | 8.706 | 83.603 | 1.00 | 0.00 | xxxx | 2429 |
| HETATM | 2430 | O | HOH | S | 38 | 104.304 | −4.180 | 89.612 | 1.00 | 0.00 | xxxx | 2430 |
| HETATM | 2431 | O | HOH | S | 39 | 87.731 | 8.186 | 72.461 | 1.00 | 0.00 | xxxx | 2431 |
| HETATM | 2432 | O | HOH | S | 40 | 63.628 | 17.345 | 87.950 | 1.00 | 0.00 | xxxx | 2432 |
| HETATM | 2433 | O | HOH | S | 41 | 79.089 | −10.881 | 79.735 | 1.00 | 0.00 | xxxx | 2433 |
| HETATM | 2434 | O | HOH | S | 42 | 85.636 | −13.717 | 77.361 | 1.00 | 0.00 | xxxx | 2434 |
| HETATM | 2435 | O | HOH | S | 43 | 82.637 | −1.836 | 67.270 | 1.00 | 0.00 | xxxx | 2435 |
| HETATM | 2436 | O | HOH | S | 44 | 83.873 | −13.593 | 84.483 | 1.00 | 0.00 | xxxx | 2436 |
| HETATM | 2437 | O | HOH | S | 45 | 58.492 | 6.346 | 98.905 | 1.00 | 0.00 | xxxx | 2437 |
| HETATM | 2438 | O | HOH | S | 46 | 102.773 | −0.145 | 91.513 | 1.00 | 0.00 | xxxx | 2438 |
| HETATM | 2439 | O | HOH | S | 47 | 95.927 | −3.173 | 63.290 | 1.00 | 0.00 | xxxx | 2439 |
| HETATM | 2440 | O | HOH | S | 48 | 95.651 | 2.686 | 96.406 | 1.00 | 0.00 | xxxx | 2440 |
| HETATM | 2441 | O | HOH | S | 49 | 94.768 | 8.045 | 90.644 | 1.00 | 0.00 | xxxx | 2441 |
| HETATM | 2442 | O | HOH | S | 50 | 110.557 | −0.277 | 81.899 | 1.00 | 0.00 | xxxx | 2442 |
| HETATM | 2443 | O | HOH | S | 51 | 89.666 | −8.773 | 94.962 | 1.00 | 0.00 | xxxx | 2443 |
| HETATM | 2444 | O | HOH | S | 52 | 78.271 | 11.349 | 100.263 | 1.00 | 0.00 | xxxx | 2444 |
| HETATM | 2445 | O | HOH | S | 53 | 90.013 | 14.373 | 72.022 | 1.00 | 0.00 | xxxx | 2445 |
| HETATM | 2446 | O | HOH | S | 54 | 54.686 | −1.991 | 79.584 | 1.00 | 0.00 | xxxx | 2446 |
| HETATM | 2447 | O | HOH | S | 55 | 54.060 | 10.208 | 97.384 | 1.00 | 0.00 | xxxx | 2447 |
| HETATM | 2448 | O | HOH | S | 56 | 59.790 | 16.417 | 80.364 | 1.00 | 0.00 | xxxx | 2448 |
| HETATM | 2449 | O | HOH | S | 57 | 77.401 | 7.520 | 100.770 | 1.00 | 0.00 | xxxx | 2449 |
| HETATM | 2450 | O | HOH | S | 58 | 51.231 | 7.595 | 85.998 | 1.00 | 0.00 | xxxx | 2450 |
| HETATM | 2451 | O | HOH | S | 59 | 82.426 | −6.107 | 67.060 | 1.00 | 0.00 | xxxx | 2451 |
| HETATM | 2452 | O | HOH | S | 60 | 68.071 | −3.957 | 74.775 | 1.00 | 0.00 | xxxx | 2452 |
| HETATM | 2453 | O | HOH | S | 61 | 61.816 | −7.423 | 86.024 | 1.00 | 0.00 | xxxx | 2453 |
| HETATM | 2454 | O | HOH | S | 62 | 62.403 | −8.118 | 88.651 | 1.00 | 0.00 | xxxx | 2454 |
| HETATM | 2455 | O | HOH | S | 63 | 91.608 | −17.642 | 73.896 | 1.00 | 0.00 | xxxx | 2455 |
| HETATM | 2456 | O | HOH | S | 64 | 78.078 | −8.136 | 83.415 | 1.00 | 0.00 | xxxx | 2456 |
| HETATM | 2457 | O | HOH | S | 65 | 64.474 | −4.069 | 98.736 | 1.00 | 0.00 | xxxx | 2457 |
| HETATM | 2458 | O | HOH | S | 66 | 102.228 | 10.529 | 75.052 | 1.00 | 0.00 | xxxx | 2458 |
| HETATM | 2459 | O | HOH | S | 67 | 78.259 | 7.764 | 74.423 | 1.00 | 0.00 | xxxx | 2459 |
| HETATM | 2460 | O | HOH | S | 68 | 50.503 | −1.277 | 89.513 | 1.00 | 0.00 | xxxx | 2460 |
| HETATM | 2461 | O | HOH | S | 69 | 104.992 | −1.785 | 90.522 | 1.00 | 0.00 | xxxx | 2461 |
| HETATM | 2462 | O | HOH | S | 70 | 91.823 | −11.886 | 88.298 | 1.00 | 0.00 | xxxx | 2462 |
| HETATM | 2463 | O | HOH | S | 71 | 99.237 | −12.571 | 86.653 | 1.00 | 0.00 | xxxx | 2463 |
| HETATM | 2464 | O | HOH | S | 72 | 55.772 | 13.625 | 95.460 | 1.00 | 0.00 | xxxx | 2464 |
| HETATM | 2465 | O | HOH | S | 73 | 102.895 | 6.060 | 66.290 | 1.00 | 0.00 | xxxx | 2465 |
| HETATM | 2466 | O | HOH | S | 74 | 101.101 | 10.601 | 72.687 | 1.00 | 0.00 | xxxx | 2466 |
| HETATM | 2467 | O | HOH | S | 75 | 90.600 | 1.190 | 62.778 | 1.00 | 0.00 | xxxx | 2467 |
| HETATM | 2468 | O | HOH | S | 76 | 46.025 | 12.655 | 90.998 | 1.00 | 0.00 | xxxx | 2468 |
| HETATM | 2469 | O | HOH | S | 77 | 89.016 | 5.541 | 89.546 | 1.00 | 0.00 | xxxx | 2469 |
| HETATM | 2470 | O | HOH | S | 78 | 55.575 | 1.314 | 97.525 | 1.00 | 0.00 | xxxx | 2470 |
| HETATM | 2471 | O | HOH | S | 79 | 106.870 | −11.908 | 75.592 | 1.00 | 0.00 | xxxx | 2471 |
| HETATM | 2472 | O | HOH | S | 80 | 106.021 | 5.208 | 86.640 | 1.00 | 0.00 | xxxx | 2472 |
| HETATM | 2473 | O | HOH | S | 81 | 85.474 | 5.243 | 66.632 | 1.00 | 0.00 | xxxx | 2473 |
| HETATM | 2474 | O | HOH | S | 82 | 108.949 | 2.324 | 81.288 | 1.00 | 0.00 | xxxx | 2474 |
| HETATM | 2475 | O | HOH | S | 83 | 93.941 | −13.080 | 68.593 | 1.00 | 0.00 | xxxx | 2475 |
| HETATM | 2476 | O | HOH | S | 84 | 98.669 | 11.594 | 80.048 | 1.00 | 0.00 | xxxx | 2476 |
| HETATM | 2477 | O | HOH | S | 85 | 105.364 | 8.545 | 80.194 | 1.00 | 0.00 | xxxx | 2477 |
| HETATM | 2478 | O | HOH | S | 86 | 76.737 | 1.253 | 103.989 | 1.00 | 0.00 | xxxx | 2478 |
| HETATM | 2479 | O | HOH | S | 87 | 53.290 | 10.371 | 100.636 | 1.00 | 0.00 | xxxx | 2479 |
| HETATM | 2480 | O | HOH | S | 88 | 102.105 | −8.780 | 86.538 | 1.00 | 0.00 | xxxx | 2480 |
| HETATM | 2481 | O | HOH | S | 89 | 108.979 | 2.590 | 87.807 | 1.00 | 0.00 | xxxx | 2481 |
| HETATM | 2482 | O | HOH | S | 90 | 101.811 | −4.625 | 66.928 | 1.00 | 0.00 | xxxx | 2482 |
| HETATM | 2483 | O | HOH | S | 91 | 86.952 | −8.105 | 61.791 | 1.00 | 0.00 | xxxx | 2483 |
| HETATM | 2484 | O | HOH | S | 92 | 75.709 | −11.553 | 83.231 | 1.00 | 0.00 | xxxx | 2484 |
| HETATM | 2485 | O | HOH | S | 93 | 69.623 | 1.107 | 105.573 | 1.00 | 0.00 | xxxx | 2485 |
| HETATM | 2486 | O | HOH | S | 94 | 56.876 | 15.613 | 94.651 | 1.00 | 0.00 | xxxx | 2486 |
| HETATM | 2487 | O | HOH | S | 95 | 77.223 | −1.392 | 104.142 | 1.00 | 0.00 | xxxx | 2487 |
| HETATM | 2488 | O | HOH | S | 96 | 68.977 | 14.567 | 81.003 | 1.00 | 0.00 | xxxx | 2488 |
| HETATM | 2489 | O | HOH | S | 97 | 103.530 | 8.231 | 87.832 | 1.00 | 0.00 | xxxx | 2489 |
| HETATM | 2490 | O | HOH | S | 98 | 53.398 | 16.767 | 91.322 | 1.00 | 0.00 | xxxx | 2490 |
| HETATM | 2491 | O | HOH | S | 99 | 66.904 | 10.170 | 74.460 | 1.00 | 0.00 | xxxx | 2491 |
| HETATM | 2492 | O | HOH | S | 100 | 66.427 | 6.432 | 109.335 | 1.00 | 0.00 | xxxx | 2492 |
| HETATM | 2493 | O | HOH | S | 101 | 51.878 | 17.923 | 84.653 | 1.00 | 0.00 | xxxx | 2493 |
| HETATM | 2494 | O | HOH | S | 102 | 69.516 | −7.765 | 98.830 | 1.00 | 0.00 | xxxx | 2494 |
| HETATM | 2495 | O | HOH | S | 103 | 71.156 | −6.045 | 97.297 | 1.00 | 0.00 | xxxx | 2495 |
| HETATM | 2496 | O | HOH | S | 104 | 92.484 | 1.110 | 96.863 | 1.00 | 0.00 | xxxx | 2496 |
| HETATM | 2497 | O | HOH | S | 105 | 104.523 | −7.523 | 86.656 | 1.00 | 0.00 | xxxx | 2497 |
| HETATM | 2498 | O | HOH | S | 106 | 53.427 | 0.356 | 94.420 | 1.00 | 0.00 | xxxx | 2498 |
| HETATM | 2499 | O | HOH | S | 107 | 92.617 | −5.406 | 61.831 | 1.00 | 0.00 | xxxx | 2499 |
| HETATM | 2500 | O | HOH | S | 108 | 54.889 | −4.851 | 79.692 | 1.00 | 0.00 | xxxx | 2500 |
| HETATM | 2501 | O | HOH | S | 109 | 100.399 | −5.002 | 64.314 | 1.00 | 0.00 | xxxx | 2501 |
| HETATM | 2502 | O | HOH | S | 110 | 66.574 | 15.836 | 85.863 | 1.00 | 0.00 | xxxx | 2502 |
| HETATM | 2503 | O | HOH | S | 111 | 89.301 | −16.735 | 85.704 | 1.00 | 0.00 | xxxx | 2503 |
| HETATM | 2504 | O | HOH | S | 112 | 76.257 | −2.694 | 101.852 | 1.00 | 0.00 | xxxx | 2504 |
| HETATM | 2505 | O | HOH | S | 113 | 76.407 | 13.842 | 79.480 | 1.00 | 0.00 | xxxx | 2505 |
| HETATM | 2506 | O | HOH | S | 114 | 77.812 | −8.591 | 71.367 | 1.00 | 0.00 | xxxx | 2506 |
| HETATM | 2507 | O | HOH | S | 115 | 62.041 | 10.695 | 106.347 | 1.00 | 0.00 | xxxx | 2507 |
| HETATM | 2508 | O | HOH | S | 116 | 87.607 | 2.895 | 60.722 | 1.00 | 0.00 | xxxx | 2508 |
| HETATM | 2509 | O | HOH | S | 117 | 51.846 | −1.707 | 79.894 | 1.00 | 0.00 | xxxx | 2509 |
| HETATM | 2510 | O | HOH | S | 118 | 96.615 | 13.271 | 68.468 | 1.00 | 0.00 | xxxx | 2510 |
| HETATM | 2511 | O | HOH | S | 119 | 68.883 | −4.447 | 104.456 | 1.00 | 0.00 | xxxx | 2511 |
| HETATM | 2512 | O | HOH | S | 120 | 78.507 | 14.813 | 89.197 | 1.00 | 0.00 | xxxx | 2512 |
| HETATM | 2513 | O | HOH | S | 121 | 53.335 | 12.886 | 98.107 | 1.00 | 0.00 | xxxx | 2513 |
| HETATM | 2514 | O | HOH | S | 122 | 56.296 | −5.318 | 85.105 | 1.00 | 0.00 | xxxx | 2514 |
| HETATM | 2515 | O | HOH | S | 123 | 88.573 | −1.610 | 98.089 | 1.00 | 0.00 | xxxx | 2515 |
| HETATM | 2516 | O | HOH | S | 124 | 106.958 | 2.835 | 68.585 | 1.00 | 0.00 | xxxx | 2516 |
| HETATM | 2517 | O | HOH | S | 125 | 91.128 | −0.551 | 95.036 | 1.00 | 0.00 | xxxx | 2517 |
| HETATM | 2518 | O | HOH | S | 126 | 87.174 | 11.082 | 80.269 | 1.00 | 0.00 | xxxx | 2518 |
| HETATM | 2519 | O | HOH | S | 127 | 68.707 | 10.512 | 72.620 | 1.00 | 0.00 | xxxx | 2519 |
| HETATM | 2520 | O | HOH | S | 128 | 98.671 | −0.829 | 64.980 | 1.00 | 0.00 | xxxx | 2520 |
| HETATM | 2521 | O | HOH | S | 129 | 65.315 | 15.486 | 79.330 | 1.00 | 0.00 | xxxx | 2521 |
| HETATM | 2522 | O | HOH | S | 130 | 80.811 | −3.719 | 67.559 | 1.00 | 0.00 | xxxx | 2522 |
| HETATM | 2523 | O | HOH | S | 131 | 90.555 | 10.401 | 64.329 | 1.00 | 0.00 | xxxx | 2523 |
| HETATM | 2524 | O | HOH | S | 132 | 103.855 | 1.624 | 92.996 | 1.00 | 0.00 | xxxx | 2524 |
| HETATM | 2525 | O | HOH | S | 133 | 83.476 | −11.583 | 89.782 | 1.00 | 0.00 | xxxx | 2525 |
| HETATM | 2526 | O | HOH | S | 134 | 64.555 | −6.773 | 98.920 | 1.00 | 0.00 | xxxx | 2526 |
| HETATM | 2527 | O | HOH | S | 135 | 92.827 | 13.344 | 66.990 | 1.00 | 0.00 | xxxx | 2527 |
| HETATM | 2528 | O | HOH | S | 136 | 79.777 | 10.104 | 78.338 | 1.00 | 0.00 | xxxx | 2528 |
| HETATM | 2529 | O | HOH | S | 137 | 111.897 | −1.398 | 79.647 | 1.00 | 0.00 | xxxx | 2529 |
| HETATM | 2530 | O | HOH | S | 138 | 71.797 | 16.681 | 93.656 | 1.00 | 0.00 | xxxx | 2530 |
| HETATM | 2531 | O | HOH | S | 139 | 85.233 | −15.957 | 83.898 | 1.00 | 0.00 | xxxx | 2531 |
| HETATM | 2532 | O | HOH | S | 140 | 65.068 | −1.301 | 77.154 | 1.00 | 0.00 | xxxx | 2532 |
| HETATM | 2533 | O | HOH | S | 141 | 97.222 | −19.132 | 71.009 | 1.00 | 0.00 | xxxx | 2533 |
| HETATM | 2534 | O | HOH | S | 142 | 78.384 | −12.495 | 76.563 | 1.00 | 0.00 | xxxx | 2534 |
| HETATM | 2535 | O | HOH | S | 143 | 86.982 | 8.225 | 70.003 | 1.00 | 0.00 | xxxx | 2535 |
| HETATM | 2536 | O | HOH | S | 144 | 95.144 | 19.750 | 70.827 | 1.00 | 0.00 | xxxx | 2536 |
| HETATM | 2537 | O | HOH | S | 145 | 53.003 | 4.985 | 80.845 | 1.00 | 0.00 | xxxx | 2537 |
| HETATM | 2538 | O | HOH | S | 146 | 59.733 | 3.617 | 106.080 | 1.00 | 0.00 | xxxx | 2538 |
| HETATM | 2539 | O | HOH | S | 147 | 55.600 | 4.084 | 79.973 | 1.00 | 0.00 | xxxx | 2539 |
| HETATM | 2540 | O | HOH | S | 148 | 70.047 | 13.700 | 84.283 | 1.00 | 0.00 | xxxx | 2540 |
| HETATM | 2541 | O | HOH | S | 149 | 64.010 | −3.472 | 96.174 | 1.00 | 0.00 | xxxx | 2541 |
| HETATM | 2542 | O | HOH | S | 150 | 76.709 | 15.830 | 97.974 | 1.00 | 0.00 | xxxx | 2542 |
| HETATM | 2543 | O | HOH | S | 151 | 87.946 | 12.867 | 72.908 | 1.00 | 0.00 | xxxx | 2543 |
| HETATM | 2544 | O | HOH | S | 152 | 77.629 | 11.855 | 77.995 | 1.00 | 0.00 | xxxx | 2544 |
| HETATM | 2545 | O | HOH | S | 153 | 81.571 | −9.261 | 90.403 | 1.00 | 0.00 | xxxx | 2545 |
| HETATM | 2546 | O | HOH | S | 154 | 86.206 | −15.823 | 79.020 | 1.00 | 0.00 | xxxx | 2546 |
| HETATM | 2547 | O | HOH | S | 155 | 59.732 | 1.153 | 76.797 | 1.00 | 0.00 | xxxx | 2547 |
| HETATM | 2548 | O | HOH | S | 156 | 62.121 | −2.955 | 94.978 | 1.00 | 0.00 | xxxx | 2548 |
| HETATM | 2549 | O | HOH | S | 157 | 89.746 | −21.133 | 79.527 | 1.00 | 0.00 | xxxx | 2549 |
| HETATM | 2550 | O | HOH | S | 158 | 50.330 | 17.743 | 88.856 | 1.00 | 0.00 | xxxx | 2550 |
| HETATM | 2551 | O | HOH | S | 159 | 112.731 | −8.840 | 80.143 | 1.00 | 0.00 | xxxx | 2551 |
| HETATM | 2552 | O | HOH | S | 160 | 77.512 | −0.547 | 70.389 | 1.00 | 0.00 | xxxx | 2552 |
| HETATM | 2553 | O | HOH | S | 161 | 107.467 | −0.669 | 90.868 | 1.00 | 0.00 | xxxx | 2553 |
| HETATM | 2554 | O | HOH | S | 162 | 80.928 | 6.192 | 74.700 | 1.00 | 0.00 | xxxx | 2554 |
| HETATM | 2555 | O | HOH | S | 163 | 101.626 | 13.065 | 71.737 | 1.00 | 0.00 | xxxx | 2555 |
| HETATM | 2556 | O | HOH | S | 164 | 66.816 | −8.640 | 98.431 | 1.00 | 0.00 | xxxx | 2556 |
| HETATM | 2557 | O | HOH | S | 165 | 52.660 | 7.548 | 81.180 | 1.00 | 0.00 | xxxx | 2557 |
| HETATM | 2558 | O | HOH | S | 166 | 97.162 | 12.117 | 78.436 | 1.00 | 0.00 | xxxx | 2558 |
| HETATM | 2559 | O | HOH | S | 167 | 100.084 | 13.362 | 78.661 | 1.00 | 0.00 | xxxx | 2559 |
| HETATM | 2560 | O | HOH | S | 168 | 112.469 | 3.315 | 71.207 | 1.00 | 0.00 | xxxx | 2560 |
| HETATM | 2561 | O | HOH | S | 169 | 108.102 | 0.594 | 88.724 | 1.00 | 0.00 | xxxx | 2561 |
| HETATM | 2562 | O | HOH | S | 170 | 83.349 | 1.004 | 67.252 | 1.00 | 0.00 | xxxx | 2562 |
| HETATM | 2563 | O | HOH | S | 171 | 82.593 | 1.925 | 100.618 | 1.00 | 0.00 | xxxx | 2563 |
| HETATM | 2564 | O | HOH | S | 172 | 48.628 | 5.314 | 90.692 | 1.00 | 0.00 | xxxx | 2564 |
| HETATM | 2565 | O | HOH | S | 173 | 61.875 | −5.472 | 104.466 | 1.00 | 0.00 | xxxx | 2565 |
| HETATM | 2566 | O | HOH | S | 174 | 100.676 | −10.021 | 88.842 | 1.00 | 0.00 | xxxx | 2566 |
| HETATM | 2567 | O | HOH | S | 175 | 55.519 | 15.322 | 78.594 | 1.00 | 0.00 | xxxx | 2567 |
| HETATM | 2568 | O | HOH | S | 176 | 86.065 | −0.576 | 97.523 | 1.00 | 0.00 | xxxx | 2568 |
| HETATM | 2569 | O | HOH | S | 177 | 97.201 | −12.062 | 59.918 | 1.00 | 0.00 | xxxx | 2569 |
| HETATM | 2570 | O | HOH | S | 178 | 47.342 | 5.542 | 85.607 | 1.00 | 0.00 | xxxx | 2570 |
| HETATM | 2571 | O | HOH | S | 179 | 76.468 | −12.251 | 74.660 | 1.00 | 0.00 | xxxx | 2571 |
| HETATM | 2572 | O | HOH | S | 180 | 57.423 | 18.345 | 88.406 | 1.00 | 0.00 | xxxx | 2572 |
| HETATM | 2573 | O | HOH | S | 181 | 80.597 | −2.205 | 99.237 | 1.00 | 0.00 | xxxx | 2573 |
| HETATM | 2574 | O | HOH | S | 182 | 103.774 | −12.854 | 69.894 | 1.00 | 0.00 | xxxx | 2574 |
| HETATM | 2575 | O | HOH | S | 183 | 72.156 | −11.814 | 94.679 | 1.00 | 0.00 | xxxx | 2575 |
| HETATM | 2576 | O | HOH | S | 184 | 81.554 | −13.910 | 83.030 | 1.00 | 0.00 | xxxx | 2576 |
| HETATM | 2577 | O | HOH | S | 185 | 95.027 | 12.134 | 77.203 | 1.00 | 0.00 | xxxx | 2577 |
| HETATM | 2578 | O | HOH | S | 186 | 68.570 | 3.287 | 109.073 | 1.00 | 0.00 | xxxx | 2578 |
| HETATM | 2579 | O | HOH | S | 187 | 51.200 | 7.008 | 83.661 | 1.00 | 0.00 | xxxx | 2579 |
| HETATM | 2580 | O | HOH | S | 188 | 61.837 | −9.774 | 76.675 | 1.00 | 0.00 | xxxx | 2580 |
| HETATM | 2581 | O | HOH | S | 189 | 86.757 | 8.270 | 84.402 | 1.00 | 0.00 | xxxx | 2581 |
| HETATM | 2582 | O | HOH | S | 190 | 49.987 | 3.480 | 94.412 | 1.00 | 0.00 | xxxx | 2582 |
| HETATM | 2583 | O | HOH | S | 191 | 91.500 | 2.394 | 79.859 | 1.00 | 0.00 | xxxx | 2583 |
| HETATM | 2584 | O | HOH | S | 192 | 97.145 | −21.675 | 79.236 | 1.00 | 0.00 | xxxx | 2584 |
| HETATM | 2585 | O | HOH | S | 193 | 87.618 | −4.559 | 62.051 | 1.00 | 0.00 | xxxx | 2585 |
| HETATM | 2586 | O | HOH | S | 194 | 68.160 | 14.469 | 86.588 | 1.00 | 0.00 | xxxx | 2586 |
| HETATM | 2587 | O | HOH | S | 195 | 66.364 | −10.527 | 74.836 | 1.00 | 0.00 | xxxx | 2587 |
| HETATM | 2588 | O | HOH | S | 196 | 74.004 | −10.006 | 96.016 | 1.00 | 0.00 | xxxx | 2588 |
| HETATM | 2589 | O | HOH | S | 197 | 82.968 | −13.859 | 87.332 | 1.00 | 0.00 | xxxx | 2589 |
| HETATM | 2590 | O | HOH | S | 198 | 108.974 | 5.718 | 85.007 | 1.00 | 0.00 | xxxx | 2590 |
| HETATM | 2591 | O | HOH | S | 199 | 92.041 | 7.360 | 91.914 | 1.00 | 0.00 | xxxx | 2591 |
| HETATM | 2592 | O | HOH | S | 200 | 82.751 | −1.455 | 100.240 | 1.00 | 0.00 | xxxx | 2592 |
| HETATM | 2593 | O | HOH | S | 201 | 99.141 | 11.984 | 83.418 | 1.00 | 0.00 | xxxx | 2593 |
| HETATM | 2594 | O | HOH | S | 202 | 49.427 | 14.666 | 93.898 | 1.00 | 0.00 | xxxx | 2594 |
| HETATM | 2595 | O | HOH | S | 203 | 83.900 | −7.265 | 93.778 | 1.00 | 0.00 | xxxx | 2595 |
| HETATM | 2596 | O | HOH | S | 204 | 61.447 | 5.704 | 104.520 | 1.00 | 0.00 | xxxx | 2596 |
| HETATM | 2597 | O | HOH | S | 205 | 97.789 | 8.951 | 91.262 | 1.00 | 0.00 | xxxx | 2597 |
| HETATM | 2598 | O | HOH | S | 206 | 87.751 | 14.201 | 75.052 | 1.00 | 0.00 | xxxx | 2598 |
| HETATM | 2599 | O | HOH | S | 207 | 106.770 | 8.139 | 82.289 | 1.00 | 0.00 | xxxx | 2599 |
| HETATM | 2600 | O | HOH | S | 208 | 65.822 | −10.575 | 96.934 | 1.00 | 0.00 | xxxx | 2600 |
| HETATM | 2601 | O | HOH | S | 209 | 81.050 | −5.890 | 69.224 | 1.00 | 0.00 | xxxx | 2601 |
| HETATM | 2602 | O | HOH | S | 210 | 74.647 | 14.640 | 86.026 | 1.00 | 0.00 | xxxx | 2602 |
| HETATM | 2603 | O | HOH | S | 211 | 108.674 | −5.814 | 74.673 | 1.00 | 0.00 | xxxx | 2603 |
| HETATM | 2604 | O | HOH | S | 212 | 77.264 | 7.891 | 71.418 | 1.00 | 0.00 | xxxx | 2604 |
| HETATM | 2605 | O | HOH | S | 213 | 60.198 | −3.619 | 94.101 | 1.00 | 0.00 | xxxx | 2605 |
| HETATM | 2606 | O | HOH | S | 214 | 89.676 | −4.554 | 61.123 | 1.00 | 0.00 | xxxx | 2606 |
| HETATM | 2607 | O | HOH | S | 215 | 92.651 | −14.696 | 70.145 | 1.00 | 0.00 | xxxx | 2607 |
| HETATM | 2608 | O | HOH | S | 216 | 99.635 | 9.883 | 85.489 | 1.00 | 0.00 | xxxx | 2608 |
| HETATM | 2609 | O | HOH | S | 217 | 59.538 | −4.151 | 102.501 | 1.00 | 0.00 | xxxx | 2609 |
| HETATM | 2610 | O | HOH | S | 218 | 72.310 | −20.773 | 82.100 | 1.00 | 0.00 | xxxx | 2610 |
| HETATM | 2611 | O | HOH | S | 219 | 84.674 | 0.825 | 92.224 | 1.00 | 0.00 | xxxx | 2611 |
| HETATM | 2612 | O | HOH | S | 220 | 104.507 | 9.632 | 73.559 | 1.00 | 0.00 | xxxx | 2612 |
| HETATM | 2613 | O | HOH | S | 221 | 84.194 | 1.903 | 94.312 | 1.00 | 0.00 | xxxx | 2613 |
| HETATM | 2614 | O | HOH | S | 222 | 108.610 | 6.829 | 81.349 | 1.00 | 0.00 | xxxx | 2614 |
| HETATM | 2615 | O | HOH | S | 223 | 98.648 | 14.172 | 82.300 | 1.00 | 0.00 | xxxx | 2615 |
| HETATM | 2616 | O | HOH | S | 224 | 90.832 | 12.419 | 78.701 | 1.00 | 0.00 | xxxx | 2616 |
| HETATM | 2617 | O | HOH | S | 225 | 52.540 | 11.909 | 76.002 | 1.00 | 0.00 | xxxx | 2617 |
| HETATM | 2618 | O | HOH | S | 226 | 105.324 | −4.324 | 67.888 | 1.00 | 0.00 | xxxx | 2618 |
| HETATM | 2619 | O | HOH | S | 227 | 79.297 | 16.150 | 98.557 | 1.00 | 0.00 | xxxx | 2619 |
| HETATM | 2620 | O | HOH | S | 228 | 85.157 | −12.672 | 66.545 | 1.00 | 0.00 | xxxx | 2620 |
| HETATM | 2621 | O | HOH | S | 229 | 105.404 | −6.081 | 70.536 | 1.00 | 0.00 | xxxx | 2621 |
| HETATM | 2622 | O | HOH | S | 230 | 107.613 | −4.792 | 71.045 | 1.00 | 0.00 | xxxx | 2622 |
| HETATM | 2623 | O | HOH | S | 231 | 109.758 | 3.007 | 77.248 | 1.00 | 0.00 | xxxx | 2623 |
| HETATM | 2624 | O | HOH | S | 232 | 78.855 | 9.433 | 98.137 | 1.00 | 0.00 | xxxx | 2624 |
| HETATM | 2625 | O | HOH | S | 233 | 77.983 | 14.271 | 92.424 | 1.00 | 0.00 | xxxx | 2625 |
| HETATM | 2626 | O | HOH | S | 234 | 59.982 | −1.213 | 75.290 | 1.00 | 0.00 | xxxx | 2626 |
| HETATM | 2627 | O | HOH | S | 235 | 78.032 | −13.631 | 72.697 | 1.00 | 0.00 | xxxx | 2627 |
| HETATM | 2628 | O | HOH | S | 236 | 104.192 | −6.382 | 61.840 | 1.00 | 0.00 | xxxx | 2628 |
| HETATM | 2629 | O | HOH | S | 237 | 60.735 | 10.512 | 108.811 | 1.00 | 0.00 | xxxx | 2629 |
| HETATM | 2630 | O | HOH | S | 238 | 74.989 | −1.300 | 69.668 | 1.00 | 0.00 | xxxx | 2630 |
| HETATM | 2631 | O | HOH | S | 239 | 86.035 | −17.451 | 85.940 | 1.00 | 0.00 | xxxx | 2631 |
| HETATM | 2632 | O | HOH | S | 240 | 99.535 | −10.757 | 59.554 | 1.00 | 0.00 | xxxx | 2632 |
| HETATM | 2633 | O | HOH | S | 241 | 75.396 | 13.835 | 83.611 | 1.00 | 0.00 | xxxx | 2633 |
| HETATM | 2634 | O | HOH | S | 242 | 90.731 | 12.998 | 84.625 | 1.00 | 0.00 | xxxx | 2634 |
| HETATM | 2635 | O | HOH | S | 243 | 52.818 | 14.956 | 95.847 | 1.00 | 0.00 | xxxx | 2635 |
| HETATM | 2636 | O | HOH | S | 244 | 80.322 | 3.758 | 85.850 | 1.00 | 0.00 | xxxx | 2636 |
| HETATM | 2637 | O | HOH | S | 245 | 55.785 | 17.408 | 92.655 | 1.00 | 0.00 | xxxx | 2637 |
| HETATM | 2638 | O | HOH | S | 246 | 115.056 | 3.055 | 70.761 | 1.00 | 0.00 | xxxx | 2638 |
| HETATM | 2639 | O | HOH | S | 247 | 87.204 | 8.691 | 79.775 | 1.00 | 0.00 | xxxx | 2639 |
| HETATM | 2640 | O | HOH | S | 248 | 67.929 | 19.944 | 86.779 | 1.00 | 0.00 | xxxx | 2640 |
| HETATM | 2641 | O | HOH | S | 249 | 77.278 | −12.670 | 79.318 | 1.00 | 0.00 | xxxx | 2641 |
| HETATM | 2642 | O | HOH | S | 250 | 89.042 | −7.911 | 60.104 | 1.00 | 0.00 | xxxx | 2642 |
| HETATM | 2643 | O | HOH | S | 251 | 66.522 | 19.073 | 85.218 | 1.00 | 0.00 | xxxx | 2643 |
| HETATM | 2644 | O | HOH | S | 252 | 61.727 | 4.783 | 107.347 | 1.00 | 0.00 | xxxx | 2644 |
| HETATM | 2645 | O | HOH | S | 253 | 106.736 | −16.531 | 79.420 | 1.00 | 0.00 | xxxx | 2645 |
| HETATM | 2646 | O | HOH | S | 254 | 73.924 | 0.038 | 67.798 | 1.00 | 0.00 | xxxx | 2646 |
| HETATM | 2647 | O | HOH | S | 255 | 69.573 | 20.378 | 85.052 | 1.00 | 0.00 | xxxx | 2647 |
| HETATM | 2648 | O | HOH | S | 256 | 60.911 | 17.953 | 97.183 | 1.00 | 0.00 | xxxx | 2648 |
| HETATM | 2649 | O | HOH | S | 257 | 64.462 | 17.633 | 96.231 | 1.00 | 0.00 | xxxx | 2649 |
| HETATM | 2650 | O | HOH | S | 258 | 62.233 | 18.287 | 95.393 | 1.00 | 0.00 | xxxx | 2650 |
| HETATM | 2651 | O | HOH | S | 259 | 56.704 | 6.246 | 102.901 | 1.00 | 0.00 | xxxx | 2651 |
| HETATM | 2652 | O | HOH | S | 260 | 100.155 | 8.120 | 65.189 | 1.00 | 0.00 | xxxx | 2652 |
| HETATM | 2653 | O | HOH | S | 261 | 68.662 | 4.614 | 111.034 | 1.00 | 0.00 | xxxx | 2653 |
| HETATM | 2654 | O | HOH | S | 262 | 85.282 | 6.689 | 68.710 | 1.00 | 0.00 | xxxx | 2654 |
| HETATM | 2655 | O | HOH | S | 263 | 53.059 | 13.136 | 78.511 | 1.00 | 0.00 | xxxx | 2655 |
| HETATM | 2656 | O | HOH | S | 264 | 57.271 | −2.144 | 100.780 | 1.00 | 0.00 | xxxx | 2656 |
| HETATM | 2657 | O | HOH | S | 265 | 52.474 | −2.587 | 90.434 | 1.00 | 0.00 | xxxx | 2657 |
| HETATM | 2658 | O | HOH | S | 266 | 80.171 | 6.190 | 71.814 | 1.00 | 0.00 | xxxx | 2658 |
| HETATM | 2659 | O | HOH | S | 267 | 99.575 | −11.497 | 56.975 | 1.00 | 0.00 | xxxx | 2659 |
| HETATM | 2660 | O | HOH | S | 268 | 99.589 | 0.394 | 99.299 | 1.00 | 0.00 | xxxx | 2660 |
| HETATM | 2661 | O | HOH | S | 269 | 69.687 | 19.479 | 91.009 | 1.00 | 0.00 | xxxx | 2661 |
| HETATM | 2662 | O | HOH | S | 270 | 57.793 | 0.261 | 108.756 | 1.00 | 0.00 | xxxx | 2662 |
| HETATM | 2663 | O | HOH | S | 271 | 81.987 | −13.534 | 70.405 | 1.00 | 0.00 | xxxx | 2663 |
| HETATM | 2664 | O | HOH | S | 272 | 68.352 | 16.666 | 93.708 | 1.00 | 0.00 | xxxx | 2664 |
| HETATM | 2665 | O | HOH | S | 273 | 52.337 | −1.842 | 99.213 | 1.00 | 0.00 | xxxx | 2665 |
| HETATM | 2666 | O | HOH | S | 274 | 113.989 | 4.886 | 71.455 | 1.00 | 0.00 | xxxx | 2666 |
| HETATM | 2667 | O | HOH | S | 275 | 78.548 | −9.578 | 81.802 | 1.00 | 0.00 | xxxx | 2667 |
| HETATM | 2668 | O | HOH | S | 276 | 71.504 | −3.457 | 71.931 | 1.00 | 0.00 | xxxx | 2668 |
| HETATM | 2669 | O | HOH | S | 277 | 81.373 | −7.989 | 65.925 | 1.00 | 0.00 | xxxx | 2669 |
| HETATM | 2670 | O | HOH | S | 278 | 75.743 | 2.240 | 69.380 | 1.00 | 0.00 | xxxx | 2670 |
| HETATM | 2671 | O | HOH | S | 279 | 106.824 | −16.531 | 84.712 | 1.00 | 0.00 | xxxx | 2671 |
| HETATM | 2672 | O | HOH | S | 280 | 85.750 | 6.704 | 62.252 | 1.00 | 0.00 | xxxx | 2672 |
| HETATM | 2673 | O | HOH | S | 281 | 85.615 | −16.056 | 74.909 | 1.00 | 0.00 | xxxx | 2673 |
| HETATM | 2674 | O | HOH | S | 282 | 54.694 | 7.097 | 100.323 | 1.00 | 0.00 | xxxx | 2674 |
| HETATM | 2675 | O | HOH | S | 283 | 82.416 | −9.126 | 92.690 | 1.00 | 0.00 | xxxx | 2675 |
| HETATM | 2676 | O | HOH | S | 284 | 104.688 | −6.624 | 68.238 | 1.00 | 0.00 | xxxx | 2676 |
| HETATM | 2677 | O | HOH | S | 285 | 104.352 | 6.757 | 89.424 | 1.00 | 0.00 | xxxx | 2677 |
| HETATM | 2678 | O | HOH | S | 286 | 69.910 | 20.393 | 88.915 | 1.00 | 0.00 | xxxx | 2678 |
| HETATM | 2679 | O | HOH | S | 287 | 61.958 | −7.213 | 97.165 | 1.00 | 0.00 | xxxx | 2679 |
| HETATM | 2680 | O | HOH | S | 288 | 94.529 | −0.882 | 102.773 | 1.00 | 0.00 | xxxx | 2680 |
| HETATM | 2681 | O | HOH | S | 289 | 77.902 | −11.183 | 83.158 | 1.00 | 0.00 | xxxx | 2681 |
| HETATM | 2682 | O | HOH | S | 290 | 107.822 | −15.668 | 77.566 | 1.00 | 0.00 | xxxx | 2682 |
| HETATM | 2683 | O | HOH | S | 291 | 95.725 | 5.302 | 96.902 | 1.00 | 0.00 | xxxx | 2683 |
| HETATM | 2684 | O | HOH | S | 292 | 64.133 | 3.617 | 72.467 | 1.00 | 0.00 | xxxx | 2684 |
| HETATM | 2685 | O | HOH | S | 293 | 108.451 | −6.376 | 72.578 | 1.00 | 0.00 | xxxx | 2685 |
| HETATM | 2686 | O | HOH | S | 294 | 46.400 | 14.955 | 93.071 | 1.00 | 0.00 | xxxx | 2686 |
| HETATM | 2687 | O | HOH | S | 295 | 58.303 | 17.796 | 86.218 | 1.00 | 0.00 | xxxx | 2687 |
| HETATM | 2688 | O | HOH | S | 296 | 59.171 | 18.585 | 83.785 | 1.00 | 0.00 | xxxx | 2688 |
| HETATM | 2689 | O | HOH | S | 297 | 97.679 | −18.685 | 80.897 | 1.00 | 0.00 | xxxx | 2689 |
| HETATM | 2690 | O | HOH | S | 298 | 77.741 | −14.710 | 81.359 | 1.00 | 0.00 | xxxx | 2690 |
| HETATM | 2691 | O | HOH | S | 299 | 92.006 | 10.014 | 61.925 | 1.00 | 0.00 | xxxx | 2691 |
| HETATM | 2692 | O | HOH | S | 300 | 61.301 | 17.558 | 78.643 | 1.00 | 0.00 | xxxx | 2692 |
| HETATM | 2693 | O | HOH | S | 301 | 52.821 | 0.688 | 97.645 | 1.00 | 0.00 | xxxx | 2693 |
| HETATM | 2694 | O | HOH | S | 302 | 84.779 | −15.819 | 76.983 | 1.00 | 0.00 | xxxx | 2694 |
| HETATM | 2695 | O | HOH | S | 303 | 101.048 | −19.438 | 80.682 | 1.00 | 0.00 | xxxx | 2695 |
| HETATM | 2696 | O | HOH | S | 304 | 96.756 | 10.671 | 86.678 | 1.00 | 0.00 | xxxx | 2696 |
| HETATM | 2697 | O | HOH | S | 305 | 52.337 | 7.315 | 99.213 | 1.00 | 0.00 | xxxx | 2697 |
| HETATM | 2698 | O | HOH | S | 306 | 66.714 | −10.256 | 100.388 | 1.00 | 0.00 | xxxx | 2698 |
| HETATM | 2699 | O | HOH | S | 307 | 68.541 | 17.330 | 99.380 | 1.00 | 0.00 | xxxx | 2699 |
| HETATM | 2700 | O | HOH | S | 308 | 73.679 | −7.075 | 68.628 | 1.00 | 0.00 | xxxx | 2700 |
| HETATM | 2701 | O | HOH | S | 309 | 82.946 | 3.923 | 67.908 | 1.00 | 0.00 | xxxx | 2701 |
| HETATM | 2702 | O | HOH | S | 310 | 87.530 | −16.979 | 69.522 | 1.00 | 0.00 | xxxx | 2702 |
| HETATM | 2703 | O | HOH | S | 311 | 90.650 | −11.119 | 59.389 | 1.00 | 0.00 | xxxx | 2703 |
| HETATM | 2704 | O | HOH | S | 312 | 102.333 | −10.420 | 90.408 | 1.00 | 0.00 | xxxx | 2704 |
| HETATM | 2705 | O | HOH | S | 313 | 76.300 | 15.569 | 93.317 | 1.00 | 0.00 | xxxx | 2705 |
| HETATM | 2706 | O | HOH | S | 314 | 49.384 | −0.292 | 85.440 | 1.00 | 0.00 | xxxx | 2706 |
| HETATM | 2707 | O | HOH | S | 315 | 94.144 | −22.571 | 85.773 | 1.00 | 0.00 | xxxx | 2707 |
| HETATM | 2708 | O | HOH | S | 316 | 86.915 | −14.572 | 69.472 | 1.00 | 0.00 | xxxx | 2708 |
| HETATM | 2709 | O | HOH | S | 317 | 95.100 | 0.697 | 100.381 | 1.00 | 0.00 | xxxx | 2709 |
| HETATM | 2710 | O | HOH | S | 318 | 69.328 | −3.518 | 72.553 | 1.00 | 0.00 | xxxx | 2710 |
| HETATM | 2711 | O | HOH | S | 319 | 80.753 | 6.983 | 69.069 | 1.00 | 0.00 | xxxx | 2711 |
| HETATM | 2712 | O | HOH | S | 320 | 46.258 | 17.814 | 93.974 | 1.00 | 0.00 | xxxx | 2712 |
| HETATM | 2713 | O | HOH | S | 321 | 81.726 | −12.988 | 89.021 | 1.00 | 0.00 | xxxx | 2713 |
| HETATM | 2714 | O | HOH | S | 322 | 57.011 | −2.865 | 93.361 | 1.00 | 0.00 | xxxx | 2714 |
| HETATM | 2715 | O | HOH | S | 323 | 87.257 | 7.473 | 60.108 | 1.00 | 0.00 | xxxx | 2715 |
| HETATM | 2716 | O | HOH | S | 324 | 51.673 | 3.931 | 78.235 | 1.00 | 0.00 | xxxx | 2716 |
| HETATM | 2717 | O | HOH | S | 325 | 100.177 | 9.710 | 88.414 | 1.00 | 0.00 | xxxx | 2717 |

### Example 11. Crystal Structure Coordinates for a T. thermosaccharolyticum Glucose-Galactose Binding Protein: TTGGBP.17C⋅Badan (Badan Attached to F17C Mutant)

Naming is standard three-letter amino acid code.

Atom positions are provided as Cartesian coordinates, using standard Protein Databank (PDB) format. ATOM records refer to amino acid atoms; HETATM records refer to non-amino acid atoms.

Column 1: record type (ATOM or HETATM); column 2: atom number; column 3: atom name (standard naming scheme for amino acids); column 4: residue name (ATOM records), or component name (HETATM records); column 5: chain identifier (A, B, C, . . . ); column 6: amino acid residue sequence number (ATOM records), or component number (HETATM records); columns 7-9: x,y,z atomic Cartesian positional coordinates; column 10: fractional occupancy (set to 1.0 in this listing); column 11: B-factor (ignored in this listing); column 12: file identifier (ignored in this listing); column 13: line number (same as atom number in this listing).

For heteroatom (HETATM) records, the component name (column 4) is as follows:

CA, calcium
HOH, water
BAD, Badan
K, potassium
EDO, ethylene glycol

| | | | | | | | | | | | | |
|---|---|---|---|---|---|---|---|---|---|---|---|---|
| ATOM | 1 | N | MET | A | 1 | 68.218 | 89.131 | 176.853 | 1.00 | 0.00 | xxxx | 1 |
| ATOM | 2 | CA | MET | A | 1 | 68.822 | 88.646 | 175.620 | 1.00 | 0.00 | xxxx | 2 |
| ATOM | 3 | CB | MET | A | 1 | 68.234 | 87.289 | 175.227 | 1.00 | 0.00 | xxxx | 3 |
| ATOM | 4 | CG | MET | A | 1 | 68.484 | 86.177 | 176.231 | 1.00 | 0.00 | xxxx | 4 |
| ATOM | 5 | SD | MET | A | 1 | 70.237 | 85.791 | 176.409 | 1.00 | 0.00 | xxxx | 5 |
| ATOM | 6 | CE | MET | A | 1 | 70.690 | 85.435 | 174.710 | 1.00 | 0.00 | xxxx | 6 |
| ATOM | 7 | C | MET | A | 1 | 68.615 | 89.642 | 174.487 | 1.00 | 0.00 | xxxx | 7 |
| ATOM | 8 | O | MET | A | 1 | 67.712 | 90.472 | 174.536 | 1.00 | 0.00 | xxxx | 8 |
| ATOM | 9 | N | LYS | A | 2 | 69.465 | 89.548 | 173.473 | 1.00 | 0.00 | xxxx | 9 |
| ATOM | 10 | CA | LYS | A | 2 | 69.300 | 90.303 | 172.238 | 1.00 | 0.00 | xxxx | 10 |
| ATOM | 11 | CB | LYS | A | 2 | 70.554 | 90.146 | 171.376 | 1.00 | 0.00 | xxxx | 11 |
| ATOM | 12 | CG | LYS | A | 2 | 70.717 | 91.140 | 170.251 | 1.00 | 0.00 | xxxx | 12 |
| ATOM | 13 | CD | LYS | A | 2 | 72.082 | 90.940 | 169.607 | 1.00 | 0.00 | xxxx | 13 |
| ATOM | 14 | CE | LYS | A | 2 | 72.374 | 91.980 | 168.546 | 1.00 | 0.00 | xxxx | 14 |
| ATOM | 15 | NZ | LYS | A | 2 | 73.735 | 91.788 | 167.968 | 1.00 | 0.00 | xxxx | 15 |
| ATOM | 16 | C | LYS | A | 2 | 68.064 | 89.811 | 171.487 | 1.00 | 0.00 | xxxx | 16 |
| ATOM | 17 | O | LYS | A | 2 | 67.828 | 88.604 | 171.408 | 1.00 | 0.00 | xxxx | 17 |
| ATOM | 18 | N | GLN | A | 3 | 67.265 | 90.725 | 170.943 | 1.00 | 0.00 | xxxx | 18 |
| ATOM | 19 | CA | GLN | A | 3 | 66.123 | 90.302 | 170.146 | 1.00 | 0.00 | xxxx | 19 |
| ATOM | 20 | CB | GLN | A | 3 | 65.033 | 91.374 | 170.089 | 1.00 | 0.00 | xxxx | 20 |
| ATOM | 21 | CG | GLN | A | 3 | 63.827 | 90.924 | 169.251 | 1.00 | 0.00 | xxxx | 21 |
| ATOM | 22 | CD | GLN | A | 3 | 62.685 | 91.932 | 169.246 | 1.00 | 0.00 | xxxx | 22 |
| ATOM | 23 | OE1 | GLN | A | 3 | 62.731 | 92.936 | 168.535 | 1.00 | 0.00 | xxxx | 23 |
| ATOM | 24 | NE2 | GLN | A | 3 | 61.652 | 91.663 | 170.041 | 1.00 | 0.00 | xxxx | 24 |
| ATOM | 25 | C | GLN | A | 3 | 66.591 | 89.955 | 168.744 | 1.00 | 0.00 | xxxx | 25 |
| ATOM | 26 | O | GLN | A | 3 | 67.319 | 90.713 | 168.106 | 1.00 | 0.00 | xxxx | 26 |
| ATOM | 27 | N | LEU | A | 4 | 66.192 | 88.779 | 168.289 | 1.00 | 0.00 | xxxx | 27 |
| ATOM | 28 | CA | LEU | A | 4 | 66.449 | 88.350 | 166.930 | 1.00 | 0.00 | xxxx | 28 |
| ATOM | 29 | CB | LEU | A | 4 | 67.079 | 86.957 | 166.908 | 1.00 | 0.00 | xxxx | 29 |
| ATOM | 30 | CG | LEU | A | 4 | 68.443 | 86.858 | 167.587 | 1.00 | 0.00 | xxxx | 30 |
| ATOM | 31 | CD1 | LEU | A | 4 | 68.891 | 85.408 | 167.634 | 1.00 | 0.00 | xxxx | 31 |
| ATOM | 32 | CD2 | LEU | A | 4 | 69.469 | 87.729 | 166.877 | 1.00 | 0.00 | xxxx | 32 |
| ATOM | 33 | C | LEU | A | 4 | 65.126 | 88.356 | 166.199 | 1.00 | 0.00 | xxxx | 33 |
| ATOM | 34 | O | LEU | A | 4 | 64.123 | 87.874 | 166.725 | 1.00 | 0.00 | xxxx | 34 |
| ATOM | 35 | N | ASN | A | 5 | 65.123 | 88.914 | 164.994 | 1.00 | 0.00 | xxxx | 35 |
| ATOM | 36 | CA | ASN | A | 5 | 63.908 | 89.001 | 164.199 | 1.00 | 0.00 | xxxx | 36 |
| ATOM | 37 | CB | ASN | A | 5 | 63.626 | 90.440 | 163.778 | 1.00 | 0.00 | xxxx | 37 |
| ATOM | 38 | CG | ASN | A | 5 | 62.959 | 91.237 | 164.865 | 1.00 | 0.00 | xxxx | 38 |
| ATOM | 39 | OD1 | ASN | A | 5 | 61.748 | 91.138 | 165.064 | 1.00 | 0.00 | xxxx | 39 |
| ATOM | 40 | ND2 | ASN | A | 5 | 63.739 | 92.035 | 165.577 | 1.00 | 0.00 | xxxx | 40 |
| ATOM | 41 | C | ASN | A | 5 | 64.027 | 88.139 | 162.970 | 1.00 | 0.00 | xxxx | 41 |
| ATOM | 42 | O | ASN | A | 5 | 64.981 | 88.276 | 162.206 | 1.00 | 0.00 | xxxx | 42 |
| ATOM | 43 | N | ILE | A | 6 | 63.056 | 87.256 | 162.775 | 1.00 | 0.00 | xxxx | 43 |
| ATOM | 44 | CA | ILE | A | 6 | 63.039 | 86.413 | 161.588 | 1.00 | 0.00 | xxxx | 44 |
| ATOM | 45 | CB | ILE | A | 6 | 63.099 | 84.931 | 161.964 | 1.00 | 0.00 | xxxx | 45 |
| ATOM | 46 | CG1 | ILE | A | 6 | 64.396 | 84.647 | 162.738 | 1.00 | 0.00 | xxxx | 46 |
| ATOM | 47 | CD1 | ILE | A | 6 | 64.417 | 83.296 | 163.418 | 1.00 | 0.00 | xxxx | 47 |
| ATOM | 48 | CG2 | ILE | A | 6 | 62.994 | 84.065 | 160.713 | 1.00 | 0.00 | xxxx | 48 |
| ATOM | 49 | C | ILE | A | 6 | 61.792 | 86.731 | 160.780 | 1.00 | 0.00 | xxxx | 49 |
| ATOM | 50 | O | ILE | A | 6 | 60.685 | 86.727 | 161.315 | 1.00 | 0.00 | xxxx | 50 |
| ATOM | 51 | N | GLY | A | 7 | 61.977 | 87.040 | 159.498 | 1.00 | 0.00 | xxxx | 51 |
| ATOM | 52 | CA | GLY | A | 7 | 60.844 | 87.251 | 158.614 | 1.00 | 0.00 | xxxx | 52 |
| ATOM | 53 | C | GLY | A | 7 | 60.351 | 85.930 | 158.044 | 1.00 | 0.00 | xxxx | 53 |
| ATOM | 54 | O | GLY | A | 7 | 61.110 | 85.211 | 157.403 | 1.00 | 0.00 | xxxx | 54 |
| ATOM | 55 | N | VAL | A | 8 | 59.086 | 85.596 | 158.285 | 1.00 | 0.00 | xxxx | 55 |
| ATOM | 56 | CA | VAL | A | 8 | 58.543 | 84.323 | 157.803 | 1.00 | 0.00 | xxxx | 56 |
| ATOM | 57 | CB | VAL | A | 8 | 58.067 | 83.416 | 158.961 | 1.00 | 0.00 | xxxx | 57 |
| ATOM | 58 | CG1 | VAL | A | 8 | 57.613 | 82.063 | 158.425 | 1.00 | 0.00 | xxxx | 58 |
| ATOM | 59 | CG2 | VAL | A | 8 | 59.164 | 83.248 | 160.003 | 1.00 | 0.00 | xxxx | 59 |
| ATOM | 60 | C | VAL | A | 8 | 57.389 | 84.593 | 156.862 | 1.00 | 0.00 | xxxx | 60 |
| ATOM | 61 | O | VAL | A | 8 | 56.422 | 85.258 | 157.249 | 1.00 | 0.00 | xxxx | 61 |
| ATOM | 62 | N | ALA | A | 9 | 57.485 | 84.095 | 155.633 | 1.00 | 0.00 | xxxx | 62 |
| ATOM | 63 | CA | ALA | A | 9 | 56.372 | 84.227 | 154.688 | 1.00 | 0.00 | xxxx | 63 |
| ATOM | 64 | CB | ALA | A | 9 | 56.826 | 84.935 | 153.410 | 1.00 | 0.00 | xxxx | 64 |
| ATOM | 65 | C | ALA | A | 9 | 55.789 | 82.854 | 154.364 | 1.00 | 0.00 | xxxx | 65 |
| ATOM | 66 | O | ALA | A | 9 | 56.511 | 81.948 | 153.948 | 1.00 | 0.00 | xxxx | 66 |
| ATOM | 67 | N | ILE | A | 10 | 54.479 | 82.709 | 154.583 | 1.00 | 0.00 | xxxx | 67 |
| ATOM | 68 | CA | ILE | A | 10 | 53.745 | 81.456 | 154.338 | 1.00 | 0.00 | xxxx | 68 |
| ATOM | 69 | CB | ILE | A | 10 | 52.776 | 81.151 | 155.498 | 1.00 | 0.00 | xxxx | 69 |
| ATOM | 70 | CG1 | ILE | A | 10 | 53.533 | 81.091 | 156.827 | 1.00 | 0.00 | xxxx | 70 |
| ATOM | 71 | CD1 | ILE | A | 10 | 54.530 | 79.969 | 156.898 | 1.00 | 0.00 | xxxx | 71 |
| ATOM | 72 | CG2 | ILE | A | 10 | 52.018 | 79.850 | 155.243 | 1.00 | 0.00 | xxxx | 72 |
| ATOM | 73 | C | ILE | A | 10 | 52.993 | 81.609 | 153.025 | 1.00 | 0.00 | xxxx | 73 |
| ATOM | 74 | O | ILE | A | 10 | 52.393 | 82.657 | 152.808 | 1.00 | 0.00 | xxxx | 74 |
| ATOM | 75 | N | TYR | A | 11 | 53.007 | 80.606 | 152.144 | 1.00 | 0.00 | xxxx | 75 |
| ATOM | 76 | CA | TYR | A | 11 | 52.490 | 80.856 | 150.790 | 1.00 | 0.00 | xxxx | 76 |
| ATOM | 77 | CB | TYR | A | 11 | 52.778 | 79.669 | 149.840 | 1.00 | 0.00 | xxxx | 77 |
| ATOM | 78 | CG | TYR | A | 11 | 51.675 | 78.630 | 149.715 | 1.00 | 0.00 | xxxx | 78 |
| ATOM | 79 | CD1 | TYR | A | 11 | 50.763 | 78.682 | 148.666 | 1.00 | 0.00 | xxxx | 79 |
| ATOM | 80 | CE1 | TYR | A | 11 | 49.747 | 77.740 | 148.540 | 1.00 | 0.00 | xxxx | 80 |
| ATOM | 81 | CZ | TYR | A | 11 | 49.635 | 76.725 | 149.476 | 1.00 | 0.00 | xxxx | 81 |
| ATOM | 82 | OH | TYR | A | 11 | 48.625 | 75.805 | 149.339 | 1.00 | 0.00 | xxxx | 82 |
| ATOM | 83 | CE2 | TYR | A | 11 | 50.532 | 76.638 | 150.529 | 1.00 | 0.00 | xxxx | 83 |
| ATOM | 84 | CD2 | TYR | A | 11 | 51.556 | 77.595 | 150.639 | 1.00 | 0.00 | xxxx | 84 |
| ATOM | 85 | C | TYR | A | 11 | 50.993 | 81.211 | 150.837 | 1.00 | 0.00 | xxxx | 85 |
| ATOM | 86 | O | TYR | A | 11 | 50.560 | 82.095 | 150.112 | 1.00 | 0.00 | xxxx | 86 |
| ATOM | 87 | N | LYS | A | 12 | 50.229 | 80.560 | 151.712 | 1.00 | 0.00 | xxxx | 87 |
| ATOM | 88 | CA | LYS | A | 12 | 48.875 | 81.018 | 152.025 | 1.00 | 0.00 | xxxx | 88 |
| ATOM | 89 | CB | LYS | A | 12 | 47.879 | 80.636 | 150.911 | 1.00 | 0.00 | xxxx | 89 |
| ATOM | 90 | CG | LYS | A | 12 | 47.377 | 79.208 | 150.906 | 1.00 | 0.00 | xxxx | 90 |
| ATOM | 91 | CD | LYS | A | 12 | 46.460 | 78.984 | 149.692 | 1.00 | 0.00 | xxxx | 91 |
| ATOM | 92 | CE | LYS | A | 12 | 45.753 | 77.645 | 149.771 | 1.00 | 0.00 | xxxx | 92 |
| ATOM | 93 | NZ | LYS | A | 12 | 44.896 | 77.401 | 148.570 | 1.00 | 0.00 | xxxx | 93 |
| ATOM | 94 | C | LYS | A | 12 | 48.454 | 80.462 | 153.383 | 1.00 | 0.00 | xxxx | 94 |
| ATOM | 95 | O | LYS | A | 12 | 48.834 | 79.349 | 153.753 | 1.00 | 0.00 | xxxx | 95 |
| ATOM | 96 | N | PHE | A | 13 | 47.707 | 81.256 | 154.148 | 1.00 | 0.00 | xxxx | 96 |
| ATOM | 97 | CA | PHE | A | 13 | 47.311 | 80.834 | 155.494 | 1.00 | 0.00 | xxxx | 97 |
| ATOM | 98 | CB | PHE | A | 13 | 46.861 | 82.032 | 156.338 | 1.00 | 0.00 | xxxx | 98 |
| ATOM | 99 | CG | PHE | A | 13 | 47.980 | 82.737 | 157.069 | 1.00 | 0.00 | xxxx | 99 |
| ATOM | 100 | CD1 | PHE | A | 13 | 49.311 | 82.494 | 156.765 | 1.00 | 0.00 | xxxx | 100 |
| ATOM | 101 | CE1 | PHE | A | 13 | 50.332 | 83.161 | 157.445 | 1.00 | 0.00 | xxxx | 101 |
| ATOM | 102 | CZ | PHE | A | 13 | 50.021 | 84.077 | 158.439 | 1.00 | 0.00 | xxxx | 102 |
| ATOM | 103 | CE2 | PHE | A | 13 | 48.697 | 84.321 | 158.754 | 1.00 | 0.00 | xxxx | 103 |
| ATOM | 104 | CD2 | PHE | A | 13 | 47.687 | 83.654 | 158.075 | 1.00 | 0.00 | xxxx | 104 |
| ATOM | 105 | C | PHE | A | 13 | 46.197 | 79.784 | 155.469 | 1.00 | 0.00 | xxxx | 105 |
| ATOM | 106 | O | PHE | A | 13 | 46.102 | 78.969 | 156.384 | 1.00 | 0.00 | xxxx | 106 |
| ATOM | 107 | N | ASP | A | 14 | 45.364 | 79.795 | 154.433 | 1.00 | 0.00 | xxxx | 107 |
| ATOM | 108 | CA | ASP | A | 14 | 44.280 | 78.822 | 154.342 | 1.00 | 0.00 | xxxx | 108 |
| ATOM | 109 | CB | ASP | A | 14 | 43.065 | 79.419 | 153.613 | 1.00 | 0.00 | xxxx | 109 |
| ATOM | 110 | CG | ASP | A | 14 | 43.364 | 79.839 | 152.179 | 1.00 | 0.00 | xxxx | 110 |
| ATOM | 111 | OD1 | ASP | A | 14 | 44.501 | 80.270 | 151.888 | 1.00 | 0.00 | xxxx | 111 |
| ATOM | 112 | OD2 | ASP | A | 14 | 42.439 | 79.751 | 151.338 | 1.00 | 0.00 | xxxx | 112 |
| ATOM | 113 | C | ASP | A | 14 | 44.742 | 77.532 | 153.659 | 1.00 | 0.00 | xxxx | 113 |
| ATOM | 114 | O | ASP | A | 14 | 44.102 | 77.032 | 152.731 | 1.00 | 0.00 | xxxx | 114 |
| ATOM | 115 | N | ASP | A | 15 | 45.873 | 77.016 | 154.128 | 1.00 | 0.00 | xxxx | 115 |
| ATOM | 116 | CA | ASP | A | 15 | 46.391 | 75.711 | 153.722 | 1.00 | 0.00 | xxxx | 116 |
| ATOM | 117 | CB | ASP | A | 15 | 47.749 | 75.858 | 153.018 | 1.00 | 0.00 | xxxx | 117 |
| ATOM | 118 | CG | ASP | A | 15 | 48.339 | 74.519 | 152.567 | 1.00 | 0.00 | xxxx | 118 |
| ATOM | 119 | OD1 | ASP | A | 15 | 48.195 | 74.183 | 151.376 | 1.00 | 0.00 | xxxx | 119 |
| ATOM | 120 | OD2 | ASP | A | 15 | 48.969 | 73.816 | 153.391 | 1.00 | 0.00 | xxxx | 120 |
| ATOM | 121 | C | ASP | A | 15 | 46.506 | 74.883 | 154.999 | 1.00 | 0.00 | xxxx | 121 |
| ATOM | 122 | O | ASP | A | 15 | 47.150 | 75.308 | 155.957 | 1.00 | 0.00 | xxxx | 122 |
| ATOM | 123 | N | THR | A | 16 | 45.874 | 73.715 | 155.023 | 1.00 | 0.00 | xxxx | 123 |
| ATOM | 124 | CA | THR | A | 16 | 45.811 | 72.911 | 156.243 | 1.00 | 0.00 | xxxx | 124 |
| ATOM | 125 | CB | THR | A | 16 | 44.916 | 71.675 | 156.040 | 1.00 | 0.00 | xxxx | 125 |
| ATOM | 126 | OG1 | THR | A | 16 | 43.607 | 72.109 | 155.636 | 1.00 | 0.00 | xxxx | 126 |
| ATOM | 127 | CG2 | THR | A | 16 | 44.800 | 70.863 | 157.334 | 1.00 | 0.00 | xxxx | 127 |
| ATOM | 128 | C | THR | A | 16 | 47.191 | 72.473 | 156.724 | 1.00 | 0.00 | xxxx | 128 |
| ATOM | 129 | O | THR | A | 16 | 47.536 | 72.673 | 157.887 | 1.00 | 0.00 | xxxx | 129 |
| ATOM | 130 | N | CYS | A | 17 | 47.992 | 71.894 | 155.838 | 1.00 | 0.00 | xxxx | 130 |
| ATOM | 131 | CA | CYS | A | 17 | 49.347 | 71.505 | 156.221 | 1.00 | 0.00 | xxxx | 131 |
| ATOM | 132 | CB | CYS | A | 17 | 50.024 | 70.780 | 155.054 | 1.00 | 0.00 | xxxx | 132 |
| ATOM | 133 | SG | CYS | A | 17 | 51.834 | 70.624 | 155.124 | 1.00 | 0.00 | xxxx | 133 |
| ATOM | 134 | C | CYS | A | 17 | 50.200 | 72.693 | 156.684 | 1.00 | 0.00 | xxxx | 134 |
| ATOM | 135 | O | CYS | A | 17 | 50.850 | 72.624 | 157.731 | 1.00 | 0.00 | xxxx | 135 |
| ATOM | 136 | N | MET | A | 18 | 50.199 | 73.789 | 155.933 | 1.00 | 0.00 | xxxx | 136 |
| ATOM | 137 | CA | MET | A | 18 | 51.103 | 74.876 | 156.280 | 1.00 | 0.00 | xxxx | 137 |
| ATOM | 138 | CB | MET | A | 18 | 51.261 | 75.855 | 155.112 | 1.00 | 0.00 | xxxx | 138 |
| ATOM | 139 | CG | MET | A | 18 | 52.133 | 75.302 | 153.980 | 1.00 | 0.00 | xxxx | 139 |
| ATOM | 140 | SD | MET | A | 18 | 53.707 | 74.616 | 154.560 | 1.00 | 0.00 | xxxx | 140 |
| ATOM | 141 | CE | MET | A | 18 | 54.419 | 76.060 | 155.321 | 1.00 | 0.00 | xxxx | 141 |
| ATOM | 142 | C | MET | A | 18 | 50.645 | 75.594 | 157.549 | 1.00 | 0.00 | xxxx | 142 |
| ATOM | 143 | O | MET | A | 18 | 51.443 | 76.258 | 158.200 | 1.00 | 0.00 | xxxx | 143 |
| ATOM | 144 | N | THR | A | 19 | 49.368 | 75.455 | 157.906 | 1.00 | 0.00 | xxxx | 144 |
| ATOM | 145 | CA | THR | A | 19 | 48.891 | 75.959 | 159.200 | 1.00 | 0.00 | xxxx | 145 |
| ATOM | 146 | CB | THR | A | 19 | 47.364 | 75.855 | 159.317 | 1.00 | 0.00 | xxxx | 146 |
| ATOM | 147 | OG1 | THR | A | 19 | 46.762 | 76.756 | 158.377 | 1.00 | 0.00 | xxxx | 147 |
| ATOM | 148 | CG2 | THR | A | 19 | 46.895 | 76.207 | 160.727 | 1.00 | 0.00 | xxxx | 148 |
| ATOM | 149 | C | THR | A | 19 | 49.567 | 75.194 | 160.331 | 1.00 | 0.00 | xxxx | 149 |
| ATOM | 150 | O | THR | A | 19 | 49.973 | 75.778 | 161.337 | 1.00 | 0.00 | xxxx | 150 |
| ATOM | 151 | N | GLY | A | 20 | 49.704 | 73.885 | 160.157 | 1.00 | 0.00 | xxxx | 151 |
| ATOM | 152 | CA | GLY | A | 20 | 50.429 | 73.080 | 161.118 | 1.00 | 0.00 | xxxx | 152 |
| ATOM | 153 | C | GLY | A | 20 | 51.880 | 73.503 | 161.211 | 1.00 | 0.00 | xxxx | 153 |
| ATOM | 154 | O | GLY | A | 20 | 52.445 | 73.588 | 162.298 | 1.00 | 0.00 | xxxx | 154 |
| ATOM | 155 | N | VAL | A | 21 | 52.492 | 73.769 | 160.062 | 1.00 | 0.00 | xxxx | 155 |
| ATOM | 156 | CA | VAL | A | 21 | 53.899 | 74.148 | 160.051 | 1.00 | 0.00 | xxxx | 156 |
| ATOM | 157 | CB | VAL | A | 21 | 54.473 | 74.174 | 158.622 | 1.00 | 0.00 | xxxx | 157 |
| ATOM | 158 | CG1 | VAL | A | 21 | 55.881 | 74.758 | 158.637 | 1.00 | 0.00 | xxxx | 158 |
| ATOM | 159 | CG2 | VAL | A | 21 | 54.492 | 72.778 | 158.037 | 1.00 | 0.00 | xxxx | 159 |
| ATOM | 160 | C | VAL | A | 21 | 54.084 | 75.495 | 160.742 | 1.00 | 0.00 | xxxx | 160 |
| ATOM | 161 | O | VAL | A | 21 | 54.950 | 75.639 | 161.606 | 1.00 | 0.00 | xxxx | 161 |
| ATOM | 162 | N | ARG | A | 22 | 53.277 | 76.492 | 160.401 | 1.00 | 0.00 | xxxx | 162 |
| ATOM | 163 | CA | ARG | A | 22 | 53.521 | 77.790 | 161.025 | 1.00 | 0.00 | xxxx | 163 |
| ATOM | 164 | CB | ARG | A | 22 | 52.779 | 78.906 | 160.292 | 1.00 | 0.00 | xxxx | 164 |
| ATOM | 165 | CG | ARG | A | 22 | 51.295 | 78.771 | 160.203 | 1.00 | 0.00 | xxxx | 165 |
| ATOM | 166 | CD | ARG | A | 22 | 50.786 | 79.812 | 159.223 | 1.00 | 0.00 | xxxx | 166 |
| ATOM | 167 | NE | ARG | A | 22 | 49.387 | 79.609 | 158.882 | 1.00 | 0.00 | xxxx | 167 |
| ATOM | 168 | CZ | ARG | A | 22 | 48.385 | 80.204 | 159.512 | 1.00 | 0.00 | xxxx | 168 |
| ATOM | 169 | NH1 | ARG | A | 22 | 48.638 | 81.038 | 160.511 | 1.00 | 0.00 | xxxx | 169 |
| ATOM | 170 | NH2 | ARG | A | 22 | 47.135 | 79.968 | 159.144 | 1.00 | 0.00 | xxxx | 170 |
| ATOM | 171 | C | ARG | A | 22 | 53.182 | 77.798 | 162.525 | 1.00 | 0.00 | xxxx | 171 |
| ATOM | 172 | O | ARG | A | 22 | 53.855 | 78.479 | 163.294 | 1.00 | 0.00 | xxxx | 172 |
| ATOM | 173 | N | ASN | A | 23 | 52.185 | 77.027 | 162.960 | 1.00 | 0.00 | xxxx | 173 |
| ATOM | 174 | CA | ASN | A | 23 | 51.922 | 76.894 | 164.395 | 1.00 | 0.00 | xxxx | 174 |
| ATOM | 175 | CB | ASN | A | 23 | 50.650 | 76.086 | 164.644 | 1.00 | 0.00 | xxxx | 175 |
| ATOM | 176 | CG | ASN | A | 23 | 49.403 | 76.841 | 164.242 | 1.00 | 0.00 | xxxx | 176 |
| ATOM | 177 | OD1 | ASN | A | 23 | 49.447 | 78.053 | 164.024 | 1.00 | 0.00 | xxxx | 177 |
| ATOM | 178 | ND2 | ASN | A | 23 | 48.283 | 76.132 | 164.141 | 1.00 | 0.00 | xxxx | 178 |
| ATOM | 179 | C | ASN | A | 23 | 53.096 | 76.244 | 165.119 | 1.00 | 0.00 | xxxx | 179 |
| ATOM | 180 | O | ASN | A | 23 | 53.463 | 76.656 | 166.221 | 1.00 | 0.00 | xxxx | 180 |
| ATOM | 181 | N | ALA | A | 24 | 53.693 | 75.234 | 164.496 | 1.00 | 0.00 | xxxx | 181 |
| ATOM | 182 | CA | ALA | A | 24 | 54.832 | 74.554 | 165.107 | 1.00 | 0.00 | xxxx | 182 |
| ATOM | 183 | CB | ALA | A | 24 | 55.132 | 73.256 | 164.378 | 1.00 | 0.00 | xxxx | 183 |
| ATOM | 184 | C | ALA | A | 24 | 56.060 | 75.467 | 165.123 | 1.00 | 0.00 | xxxx | 184 |
| ATOM | 185 | O | ALA | A | 24 | 56.841 | 75.441 | 166.077 | 1.00 | 0.00 | xxxx | 185 |
| ATOM | 186 | N | MET | A | 25 | 56.231 | 76.266 | 164.073 | 1.00 | 0.00 | xxxx | 186 |
| ATOM | 187 | CA | MET | A | 25 | 57.317 | 77.247 | 164.038 | 1.00 | 0.00 | xxxx | 187 |
| ATOM | 188 | CB | MET | A | 25 | 57.376 | 77.945 | 162.680 | 1.00 | 0.00 | xxxx | 188 |
| ATOM | 189 | CG | MET | A | 25 | 57.910 | 77.046 | 161.580 | 1.00 | 0.00 | xxxx | 189 |
| ATOM | 190 | SD | MET | A | 25 | 58.065 | 77.901 | 160.004 | 1.00 | 0.00 | xxxx | 190 |
| ATOM | 191 | CE | MET | A | 25 | 59.335 | 79.085 | 160.417 | 1.00 | 0.00 | xxxx | 191 |
| ATOM | 192 | C | MET | A | 25 | 57.157 | 78.270 | 165.152 | 1.00 | 0.00 | xxxx | 192 |
| ATOM | 193 | O | MET | A | 25 | 58.121 | 78.614 | 165.837 | 1.00 | 0.00 | xxxx | 193 |
| ATOM | 194 | N | THR | A | 26 | 55.933 | 78.746 | 165.333 | 1.00 | 0.00 | xxxx | 194 |
| ATOM | 195 | CA | THR | A | 26 | 55.656 | 79.711 | 166.388 | 1.00 | 0.00 | xxxx | 195 |
| ATOM | 196 | CB | THR | A | 26 | 54.196 | 80.194 | 166.321 | 1.00 | 0.00 | xxxx | 196 |
| ATOM | 197 | OG1 | THR | A | 26 | 53.990 | 80.881 | 165.080 | 1.00 | 0.00 | xxxx | 197 |
| ATOM | 198 | CG2 | THR | A | 26 | 53.895 | 81.138 | 167.480 | 1.00 | 0.00 | xxxx | 198 |
| ATOM | 199 | C | THR | A | 26 | 55.966 | 79.100 | 167.758 | 1.00 | 0.00 | xxxx | 199 |
| ATOM | 200 | O | THR | A | 26 | 56.518 | 79.775 | 168.636 | 1.00 | 0.00 | xxxx | 200 |
| ATOM | 201 | N | ALA | A | 27 | 55.660 | 77.814 | 167.930 | 1.00 | 0.00 | xxxx | 201 |
| ATOM | 202 | CA | ALA | A | 27 | 55.944 | 77.135 | 169.194 | 1.00 | 0.00 | xxxx | 202 |
| ATOM | 203 | CB | ALA | A | 27 | 55.284 | 75.757 | 169.219 | 1.00 | 0.00 | xxxx | 203 |
| ATOM | 204 | C | ALA | A | 27 | 57.451 | 77.008 | 169.440 | 1.00 | 0.00 | xxxx | 204 |
| ATOM | 205 | O | ALA | A | 27 | 57.929 | 77.222 | 170.555 | 1.00 | 0.00 | xxxx | 205 |
| ATOM | 206 | N | GLU | A | 28 | 58.198 | 76.661 | 168.396 | 1.00 | 0.00 | xxxx | 206 |
| ATOM | 207 | CA | GLU | A | 28 | 59.646 | 76.522 | 168.525 | 1.00 | 0.00 | xxxx | 207 |
| ATOM | 208 | CB | GLU | A | 28 | 60.236 | 75.939 | 167.244 | 1.00 | 0.00 | xxxx | 208 |
| ATOM | 209 | CG | GLU | A | 28 | 59.853 | 74.496 | 166.998 | 1.00 | 0.00 | xxxx | 209 |
| ATOM | 210 | CD | GLU | A | 28 | 60.289 | 73.586 | 168.133 | 1.00 | 0.00 | xxxx | 210 |
| ATOM | 211 | OE1 | GLU | A | 28 | 61.391 | 73.800 | 168.691 | 1.00 | 0.00 | xxxx | 211 |
| ATOM | 212 | OE2 | GLU | A | 28 | 59.520 | 72.662 | 168.472 | 1.00 | 0.00 | xxxx | 212 |
| ATOM | 213 | C | GLU | A | 28 | 60.334 | 77.847 | 168.846 | 1.00 | 0.00 | xxxx | 213 |
| ATOM | 214 | O | GLU | A | 28 | 61.358 | 77.871 | 169.533 | 1.00 | 0.00 | xxxx | 214 |
| ATOM | 215 | N | ALA | A | 29 | 59.765 | 78.941 | 168.347 | 1.00 | 0.00 | xxxx | 215 |
| ATOM | 216 | CA | ALA | A | 29 | 60.377 | 80.260 | 168.470 | 1.00 | 0.00 | xxxx | 216 |
| ATOM | 217 | CB | ALA | A | 29 | 59.882 | 81.168 | 167.363 | 1.00 | 0.00 | xxxx | 217 |
| ATOM | 218 | C | ALA | A | 29 | 60.122 | 80.920 | 169.818 | 1.00 | 0.00 | xxxx | 218 |
| ATOM | 219 | O | ALA | A | 29 | 60.847 | 81.841 | 170.197 | 1.00 | 0.00 | xxxx | 219 |
| ATOM | 220 | N | GLN | A | 30 | 59.088 | 80.469 | 170.527 | 1.00 | 0.00 | xxxx | 220 |
| ATOM | 221 | C | GLN | A | 30 | 59.944 | 81.122 | 172.747 | 1.00 | 0.00 | xxxx | 221 |
| ATOM | 222 | O | GLN | A | 30 | 60.601 | 80.114 | 173.019 | 1.00 | 0.00 | xxxx | 222 |
| ATOM | 223 | CA | GLN | A | 30 | 58.724 | 80.991 | 171.843 | 1.00 | 0.00 | xxxx | 223 |
| ATOM | 224 | CB | GLN | A | 30 | 57.685 | 80.064 | 172.494 | 1.00 | 0.00 | xxxx | 224 |
| ATOM | 225 | CG | GLN | A | 30 | 57.220 | 80.480 | 173.885 | 1.00 | 0.00 | xxxx | 225 |
| ATOM | 226 | CD | GLN | A | 30 | 56.502 | 79.356 | 174.622 | 1.00 | 0.00 | xxxx | 226 |
| ATOM | 227 | OE1 | GLN | A | 30 | 57.126 | 78.385 | 175.055 | 1.00 | 0.00 | xxxx | 227 |
| ATOM | 228 | NE2 | GLN | A | 30 | 55.186 | 79.483 | 174.768 | 1.00 | 0.00 | xxxx | 228 |
| ATOM | 229 | N | GLY | A | 31 | 60.247 | 82.330 | 173.213 | 1.00 | 0.00 | xxxx | 229 |
| ATOM | 230 | CA | GLY | A | 31 | 61.366 | 82.552 | 174.111 | 1.00 | 0.00 | xxxx | 230 |
| ATOM | 231 | C | GLY | A | 31 | 62.735 | 82.629 | 173.456 | 1.00 | 0.00 | xxxx | 231 |
| ATOM | 232 | O | GLY | A | 31 | 63.740 | 82.816 | 174.142 | 1.00 | 0.00 | xxxx | 232 |
| ATOM | 233 | N | LYS | A | 32 | 62.784 | 82.500 | 172.134 | 1.00 | 0.00 | xxxx | 233 |
| ATOM | 234 | CA | LYS | A | 32 | 64.065 | 82.426 | 171.430 | 1.00 | 0.00 | xxxx | 234 |
| ATOM | 235 | CB | LYS | A | 32 | 64.214 | 81.071 | 170.728 | 1.00 | 0.00 | xxxx | 235 |
| ATOM | 236 | CG | LYS | A | 32 | 64.340 | 79.880 | 171.664 | 1.00 | 0.00 | xxxx | 236 |
| ATOM | 237 | CD | LYS | A | 32 | 64.685 | 78.617 | 170.878 | 1.00 | 0.00 | xxxx | 237 |
| ATOM | 238 | CE | LYS | A | 32 | 64.614 | 77.382 | 171.758 | 1.00 | 0.00 | xxxx | 238 |
| ATOM | 239 | NZ | LYS | A | 32 | 63.207 | 77.104 | 172.173 | 1.00 | 0.00 | xxxx | 239 |
| ATOM | 240 | C | LYS | A | 32 | 64.248 | 83.539 | 170.403 | 1.00 | 0.00 | xxxx | 240 |
| ATOM | 241 | O | LYS | A | 32 | 65.297 | 84.184 | 170.351 | 1.00 | 0.00 | xxxx | 241 |
| ATOM | 242 | N | ALA | A | 33 | 63.233 | 83.742 | 169.573 | 1.00 | 0.00 | xxxx | 242 |
| ATOM | 243 | CA | ALA | A | 33 | 63.301 | 84.738 | 168.511 | 1.00 | 0.00 | xxxx | 243 |
| ATOM | 244 | CB | ALA | A | 33 | 63.997 | 84.168 | 167.288 | 1.00 | 0.00 | xxxx | 244 |
| ATOM | 245 | C | ALA | A | 33 | 61.908 | 85.222 | 168.148 | 1.00 | 0.00 | xxxx | 245 |
| ATOM | 246 | O | ALA | A | 33 | 60.927 | 84.505 | 168.321 | 1.00 | 0.00 | xxxx | 246 |
| ATOM | 247 | N | LYS | A | 34 | 61.829 | 86.448 | 167.648 | 1.00 | 0.00 | xxxx | 247 |
| ATOM | 248 | CA | LYS | A | 34 | 60.563 | 87.009 | 167.217 | 1.00 | 0.00 | xxxx | 248 |
| ATOM | 249 | CB | LYS | A | 34 | 60.564 | 88.530 | 167.390 | 1.00 | 0.00 | xxxx | 249 |
| ATOM | 250 | CG | LYS | A | 34 | 59.308 | 89.220 | 166.878 | 1.00 | 0.00 | xxxx | 250 |
| ATOM | 251 | CD | LYS | A | 34 | 59.300 | 90.688 | 167.274 | 1.00 | 0.00 | xxxx | 251 |
| ATOM | 252 | CE | LYS | A | 34 | 58.087 | 91.411 | 166.704 | 1.00 | 0.00 | xxxx | 252 |
| ATOM | 253 | NZ | LYS | A | 34 | 58.078 | 92.851 | 167.064 | 1.00 | 0.00 | xxxx | 253 |
| ATOM | 254 | C | LYS | A | 34 | 60.309 | 86.641 | 165.765 | 1.00 | 0.00 | xxxx | 254 |
| ATOM | 255 | O | LYS | A | 34 | 61.115 | 86.966 | 164.889 | 1.00 | 0.00 | xxxx | 255 |
| ATOM | 256 | N | LEU | A | 35 | 59.210 | 85.933 | 165.513 | 1.00 | 0.00 | xxxx | 256 |
| ATOM | 257 | CA | LEU | A | 35 | 58.799 | 85.667 | 164.139 | 1.00 | 0.00 | xxxx | 257 |
| ATOM | 258 | CB | LEU | A | 35 | 58.088 | 84.320 | 164.016 | 1.00 | 0.00 | xxxx | 258 |
| ATOM | 259 | CG | LEU | A | 35 | 58.834 | 83.075 | 164.493 | 1.00 | 0.00 | xxxx | 259 |
| ATOM | 260 | CD1 | LEU | A | 35 | 58.058 | 81.826 | 164.102 | 1.00 | 0.00 | xxxx | 260 |
| ATOM | 261 | CD2 | LEU | A | 35 | 60.256 | 83.025 | 163.946 | 1.00 | 0.00 | xxxx | 261 |
| ATOM | 262 | C | LEU | A | 35 | 57.883 | 86.779 | 163.660 | 1.00 | 0.00 | xxxx | 262 |
| ATOM | 263 | O | LEU | A | 35 | 56.903 | 87.131 | 164.328 | 1.00 | 0.00 | xxxx | 263 |
| ATOM | 264 | N | ASN | A | 36 | 58.214 | 87.342 | 162.508 | 1.00 | 0.00 | xxxx | 264 |
| ATOM | 265 | CA | ASN | A | 36 | 57.350 | 88.298 | 161.851 | 1.00 | 0.00 | xxxx | 265 |
| ATOM | 266 | CB | ASN | A | 36 | 58.146 | 89.516 | 161.395 | 1.00 | 0.00 | xxxx | 266 |
| ATOM | 267 | CG | ASN | A | 36 | 58.825 | 90.224 | 162.557 | 1.00 | 0.00 | xxxx | 267 |
| ATOM | 268 | OD1 | ASN | A | 36 | 58.214 | 91.050 | 163.229 | 1.00 | 0.00 | xxxx | 268 |
| ATOM | 269 | ND2 | ASN | A | 36 | 60.083 | 89.879 | 162.812 | 1.00 | 0.00 | xxxx | 269 |
| ATOM | 270 | C | ASN | A | 36 | 56.696 | 87.561 | 160.692 | 1.00 | 0.00 | xxxx | 270 |
| ATOM | 271 | O | ASN | A | 36 | 57.265 | 87.452 | 159.612 | 1.00 | 0.00 | xxxx | 271 |
| ATOM | 272 | N | MET | A | 37 | 55.507 | 87.023 | 160.943 | 1.00 | 0.00 | xxxx | 272 |
| ATOM | 273 | CA | MET | A | 37 | 54.933 | 86.040 | 160.028 | 1.00 | 0.00 | xxxx | 273 |
| ATOM | 274 | CB | MET | A | 37 | 54.441 | 84.820 | 160.796 | 1.00 | 0.00 | xxxx | 274 |
| ATOM | 275 | CG | MET | A | 37 | 54.241 | 83.606 | 159.905 | 1.00 | 0.00 | xxxx | 275 |
| ATOM | 276 | SD | MET | A | 37 | 53.741 | 82.201 | 160.891 | 1.00 | 0.00 | xxxx | 276 |
| ATOM | 277 | CE | MET | A | 37 | 55.317 | 81.666 | 161.542 | 1.00 | 0.00 | xxxx | 277 |
| ATOM | 278 | C | MET | A | 37 | 53.804 | 86.631 | 159.210 | 1.00 | 0.00 | xxxx | 278 |
| ATOM | 279 | O | MET | A | 37 | 52.875 | 87.236 | 159.754 | 1.00 | 0.00 | xxxx | 279 |
| ATOM | 280 | N | VAL | A | 38 | 53.891 | 86.443 | 157.900 | 1.00 | 0.00 | xxxx | 280 |
| ATOM | 281 | CA | VAL | A | 38 | 52.949 | 87.050 | 156.969 | 1.00 | 0.00 | xxxx | 281 |
| ATOM | 282 | CB | VAL | A | 38 | 53.619 | 88.173 | 156.151 | 1.00 | 0.00 | xxxx | 282 |
| ATOM | 283 | CG1 | VAL | A | 38 | 54.158 | 89.253 | 157.081 | 1.00 | 0.00 | xxxx | 283 |
| ATOM | 284 | CG2 | VAL | A | 38 | 54.728 | 87.611 | 155.267 | 1.00 | 0.00 | xxxx | 284 |
| ATOM | 285 | C | VAL | A | 38 | 52.346 | 86.011 | 156.038 | 1.00 | 0.00 | xxxx | 285 |
| ATOM | 286 | O | VAL | A | 38 | 52.937 | 84.963 | 155.770 | 1.00 | 0.00 | xxxx | 286 |
| ATOM | 287 | N | ASP | A | 39 | 51.157 | 86.340 | 155.550 | 1.00 | 0.00 | xxxx | 287 |
| ATOM | 288 | CA | ASP | A | 39 | 50.355 | 85.500 | 154.667 | 1.00 | 0.00 | xxxx | 288 |
| ATOM | 289 | CB | ASP | A | 39 | 48.890 | 85.561 | 155.133 | 1.00 | 0.00 | xxxx | 289 |
| ATOM | 290 | CG | ASP | A | 39 | 47.925 | 84.752 | 154.270 | 1.00 | 0.00 | xxxx | 290 |
| ATOM | 291 | OD1 | ASP | A | 39 | 48.357 | 84.004 | 153.366 | 1.00 | 0.00 | xxxx | 291 |
| ATOM | 292 | OD2 | ASP | A | 39 | 46.700 | 84.868 | 154.522 | 1.00 | 0.00 | xxxx | 292 |
| ATOM | 293 | C | ASP | A | 39 | 50.516 | 86.011 | 153.238 | 1.00 | 0.00 | xxxx | 293 |
| ATOM | 294 | O | ASP | A | 39 | 50.115 | 87.138 | 152.935 | 1.00 | 0.00 | xxxx | 294 |
| ATOM | 295 | N | SER | A | 40 | 51.092 | 85.197 | 152.362 | 1.00 | 0.00 | xxxx | 295 |
| ATOM | 296 | CA | SER | A | 40 | 51.324 | 85.649 | 150.988 | 1.00 | 0.00 | xxxx | 296 |
| ATOM | 297 | CB | SER | A | 40 | 52.541 | 84.930 | 150.390 | 1.00 | 0.00 | xxxx | 297 |
| ATOM | 298 | OG | SER | A | 40 | 53.719 | 85.267 | 151.122 | 1.00 | 0.00 | xxxx | 298 |
| ATOM | 299 | C | SER | A | 40 | 50.087 | 85.467 | 150.102 | 1.00 | 0.00 | xxxx | 299 |
| ATOM | 300 | O | SER | A | 40 | 50.092 | 85.867 | 148.938 | 1.00 | 0.00 | xxxx | 300 |
| ATOM | 301 | N | GLN | A | 41 | 49.018 | 84.901 | 150.661 | 1.00 | 0.00 | xxxx | 301 |
| ATOM | 302 | CA | GLN | A | 41 | 47.725 | 84.784 | 149.963 | 1.00 | 0.00 | xxxx | 302 |
| ATOM | 303 | CB | GLN | A | 41 | 47.038 | 86.155 | 149.887 | 1.00 | 0.00 | xxxx | 303 |
| ATOM | 304 | CG | GLN | A | 41 | 46.765 | 86.792 | 151.256 | 1.00 | 0.00 | xxxx | 304 |
| ATOM | 305 | CD | GLN | A | 41 | 45.986 | 88.102 | 151.146 | 1.00 | 0.00 | xxxx | 305 |
| ATOM | 306 | OE1 | GLN | A | 41 | 46.553 | 89.182 | 151.285 | 1.00 | 0.00 | xxxx | 306 |
| ATOM | 307 | NE2 | GLN | A | 41 | 44.684 | 88.004 | 150.891 | 1.00 | 0.00 | xxxx | 307 |
| ATOM | 308 | C | GLN | A | 41 | 47.842 | 84.186 | 148.553 | 1.00 | 0.00 | xxxx | 308 |
| ATOM | 309 | O | GLN | A | 41 | 47.128 | 84.599 | 147.624 | 1.00 | 0.00 | xxxx | 309 |
| ATOM | 310 | N | ASN | A | 42 | 48.747 | 83.218 | 148.412 | 1.00 | 0.00 | xxxx | 310 |
| ATOM | 311 | CA | ASN | A | 42 | 48.961 | 82.480 | 147.165 | 1.00 | 0.00 | xxxx | 311 |
| ATOM | 312 | CB | ASN | A | 42 | 47.737 | 81.614 | 146.848 | 1.00 | 0.00 | xxxx | 312 |
| ATOM | 313 | CG | ASN | A | 42 | 48.036 | 80.518 | 145.852 | 1.00 | 0.00 | xxxx | 313 |
| ATOM | 314 | OD1 | ASN | A | 42 | 49.133 | 79.957 | 145.819 | 1.00 | 0.00 | xxxx | 314 |
| ATOM | 315 | ND2 | ASN | A | 42 | 47.053 | 80.213 | 145.015 | 1.00 | 0.00 | xxxx | 315 |
| ATOM | 316 | C | ASN | A | 42 | 49.282 | 83.417 | 146.002 | 1.00 | 0.00 | xxxx | 316 |
| ATOM | 317 | O | ASN | A | 42 | 48.898 | 83.167 | 144.855 | 1.00 | 0.00 | xxxx | 317 |
| ATOM | 318 | N | SER | A | 43 | 49.993 | 84.498 | 146.311 | 1.00 | 0.00 | xxxx | 318 |
| ATOM | 319 | CA | SER | A | 43 | 50.388 | 85.490 | 145.320 | 1.00 | 0.00 | xxxx | 319 |
| ATOM | 320 | CB | SER | A | 43 | 49.588 | 86.785 | 145.512 | 1.00 | 0.00 | xxxx | 320 |
| ATOM | 321 | OG | SER | A | 43 | 50.124 | 87.836 | 144.719 | 1.00 | 0.00 | xxxx | 321 |
| ATOM | 322 | C | SER | A | 43 | 51.878 | 85.789 | 145.427 | 1.00 | 0.00 | xxxx | 322 |
| ATOM | 323 | O | SER | A | 43 | 52.328 | 86.321 | 146.445 | 1.00 | 0.00 | xxxx | 323 |
| ATOM | 324 | N | GLN | A | 44 | 52.653 | 85.458 | 144.399 | 1.00 | 0.00 | xxxx | 324 |
| ATOM | 325 | CA | GLN | A | 44 | 54.073 | 85.793 | 144.453 | 1.00 | 0.00 | xxxx | 325 |
| ATOM | 326 | CB | GLN | A | 44 | 54.850 | 85.140 | 143.310 | 1.00 | 0.00 | xxxx | 326 |
| ATOM | 327 | CG | GLN | A | 44 | 56.368 | 85.194 | 143.524 | 1.00 | 0.00 | xxxx | 327 |
| ATOM | 328 | CD | GLN | A | 44 | 56.808 | 84.502 | 144.807 | 1.00 | 0.00 | xxxx | 328 |
| ATOM | 329 | OE1 | GLN | A | 44 | 56.407 | 83.371 | 145.086 | 1.00 | 0.00 | xxxx | 329 |
| ATOM | 330 | NE2 | GLN | A | 44 | 57.639 | 85.182 | 145.599 | 1.00 | 0.00 | xxxx | 330 |
| ATOM | 331 | C | GLN | A | 44 | 54.314 | 87.315 | 144.446 | 1.00 | 0.00 | xxxx | 331 |
| ATOM | 332 | O | GLN | A | 44 | 55.211 | 87.782 | 145.147 | 1.00 | 0.00 | xxxx | 332 |
| ATOM | 333 | N | PRO | A | 45 | 53.541 | 88.087 | 143.657 | 1.00 | 0.00 | xxxx | 333 |
| ATOM | 334 | CA | PRO | A | 45 | 53.746 | 89.539 | 143.776 | 1.00 | 0.00 | xxxx | 334 |
| ATOM | 335 | CB | PRO | A | 45 | 52.693 | 90.113 | 142.824 | 1.00 | 0.00 | xxxx | 335 |
| ATOM | 336 | CG | PRO | A | 45 | 52.587 | 89.055 | 141.760 | 1.00 | 0.00 | xxxx | 336 |
| ATOM | 337 | CD | PRO | A | 45 | 52.642 | 87.762 | 142.526 | 1.00 | 0.00 | xxxx | 337 |
| ATOM | 338 | C | PRO | A | 45 | 53.549 | 90.045 | 145.204 | 1.00 | 0.00 | xxxx | 338 |
| ATOM | 339 | O | PRO | A | 45 | 54.320 | 90.882 | 145.667 | 1.00 | 0.00 | xxxx | 339 |
| ATOM | 340 | N | THR | A | 46 | 52.555 | 89.511 | 145.903 | 1.00 | 0.00 | xxxx | 340 |
| ATOM | 341 | CA | THR | A | 46 | 52.363 | 89.856 | 147.308 | 1.00 | 0.00 | xxxx | 341 |
| ATOM | 342 | CB | THR | A | 46 | 51.108 | 89.171 | 147.863 | 1.00 | 0.00 | xxxx | 342 |
| ATOM | 343 | OG1 | THR | A | 46 | 49.954 | 89.728 | 147.216 | 1.00 | 0.00 | xxxx | 343 |
| ATOM | 344 | CG2 | THR | A | 46 | 51.004 | 89.373 | 149.368 | 1.00 | 0.00 | xxxx | 344 |
| ATOM | 345 | C | THR | A | 46 | 53.581 | 89.474 | 148.151 | 1.00 | 0.00 | xxxx | 345 |
| ATOM | 346 | O | THR | A | 46 | 54.094 | 90.284 | 148.941 | 1.00 | 0.00 | xxxx | 346 |
| ATOM | 347 | N | GLN | A | 47 | 54.057 | 88.247 | 147.966 | 1.00 | 0.00 | xxxx | 347 |
| ATOM | 348 | CA | GLN | A | 47 | 55.214 | 87.777 | 148.706 | 1.00 | 0.00 | xxxx | 348 |
| ATOM | 349 | CB | GLN | A | 47 | 55.459 | 86.299 | 148.425 | 1.00 | 0.00 | xxxx | 349 |
| ATOM | 350 | CG | GLN | A | 47 | 56.539 | 85.727 | 149.310 | 1.00 | 0.00 | xxxx | 350 |
| ATOM | 351 | CD | GLN | A | 47 | 56.655 | 84.234 | 149.145 | 1.00 | 0.00 | xxxx | 351 |
| ATOM | 352 | OE1 | GLN | A | 47 | 57.354 | 83.750 | 148.255 | 1.00 | 0.00 | xxxx | 352 |
| ATOM | 353 | NE2 | GLN | A | 47 | 55.963 | 83.490 | 149.997 | 1.00 | 0.00 | xxxx | 353 |
| ATOM | 354 | C | GLN | A | 47 | 56.472 | 88.582 | 148.381 | 1.00 | 0.00 | xxxx | 354 |
| ATOM | 355 | O | GLN | A | 47 | 57.287 | 88.818 | 149.255 | 1.00 | 0.00 | xxxx | 355 |
| ATOM | 356 | N | ASN | A | 48 | 56.625 | 88.998 | 147.127 | 1.00 | 0.00 | xxxx | 356 |
| ATOM | 357 | CA | ASN | A | 48 | 57.794 | 89.793 | 146.754 | 1.00 | 0.00 | xxxx | 357 |
| ATOM | 358 | CB | ASN | A | 48 | 57.820 | 90.061 | 145.251 | 1.00 | 0.00 | xxxx | 358 |
| ATOM | 359 | CG | ASN | A | 48 | 58.164 | 88.820 | 144.434 | 1.00 | 0.00 | xxxx | 359 |
| ATOM | 360 | OD1 | ASN | A | 48 | 58.698 | 87.839 | 144.954 | 1.00 | 0.00 | xxxx | 360 |
| ATOM | 361 | ND2 | ASN | A | 48 | 57.881 | 88.879 | 143.135 | 1.00 | 0.00 | xxxx | 361 |
| ATOM | 362 | C | ASN | A | 48 | 57.802 | 91.113 | 147.527 | 1.00 | 0.00 | xxxx | 362 |
| ATOM | 363 | O | ASN | A | 48 | 58.854 | 91.563 | 147.987 | 1.00 | 0.00 | xxxx | 363 |
| ATOM | 364 | N | ASP | A | 49 | 56.626 | 91.724 | 147.670 | 1.00 | 0.00 | xxxx | 364 |
| ATOM | 365 | CA | ASP | A | 49 | 56.486 | 92.945 | 148.462 | 1.00 | 0.00 | xxxx | 365 |
| ATOM | 366 | CB | ASP | A | 49 | 55.059 | 93.504 | 148.364 | 1.00 | 0.00 | xxxx | 366 |
| ATOM | 367 | CG | ASP | A | 49 | 54.752 | 94.096 | 147.000 | 1.00 | 0.00 | xxxx | 367 |
| ATOM | 368 | OD1 | ASP | A | 49 | 55.700 | 94.468 | 146.275 | 1.00 | 0.00 | xxxx | 368 |
| ATOM | 369 | OD2 | ASP | A | 49 | 53.555 | 94.210 | 146.653 | 1.00 | 0.00 | xxxx | 369 |
| ATOM | 370 | C | ASP | A | 49 | 56.833 | 92.672 | 149.922 | 1.00 | 0.00 | xxxx | 370 |
| ATOM | 371 | O | ASP | A | 49 | 57.471 | 93.483 | 150.580 | 1.00 | 0.00 | xxxx | 371 |
| ATOM | 372 | N | GLN | A | 50 | 56.416 | 91.513 | 150.421 | 1.00 | 0.00 | xxxx | 372 |
| ATOM | 373 | CA | GLN | A | 50 | 56.674 | 91.169 | 151.817 | 1.00 | 0.00 | xxxx | 373 |
| ATOM | 374 | CB | GLN | A | 50 | 55.875 | 89.922 | 152.210 | 1.00 | 0.00 | xxxx | 374 |
| ATOM | 375 | CG | GLN | A | 50 | 54.378 | 90.222 | 152.317 | 1.00 | 0.00 | xxxx | 375 |
| ATOM | 376 | CD | GLN | A | 50 | 53.499 | 88.981 | 152.264 | 1.00 | 0.00 | xxxx | 376 |
| ATOM | 377 | OE1 | GLN | A | 50 | 53.886 | 87.941 | 151.732 | 1.00 | 0.00 | xxxx | 377 |
| ATOM | 378 | NE2 | GLN | A | 50 | 52.298 | 89.098 | 152.816 | 1.00 | 0.00 | xxxx | 378 |
| ATOM | 379 | C | GLN | A | 50 | 58.164 | 90.962 | 152.055 | 1.00 | 0.00 | xxxx | 379 |
| ATOM | 380 | O | GLN | A | 50 | 58.705 | 91.402 | 153.067 | 1.00 | 0.00 | xxxx | 380 |
| ATOM | 381 | N | VAL | A | 51 | 58.823 | 90.294 | 151.112 | 1.00 | 0.00 | xxxx | 381 |
| ATOM | 382 | CA | VAL | A | 51 | 60.260 | 90.081 | 151.211 | 1.00 | 0.00 | xxxx | 382 |
| ATOM | 383 | CB | VAL | A | 51 | 60.764 | 89.156 | 150.098 | 1.00 | 0.00 | xxxx | 383 |
| ATOM | 384 | CG1 | VAL | A | 51 | 62.308 | 89.153 | 150.045 | 1.00 | 0.00 | xxxx | 384 |
| ATOM | 385 | CG2 | VAL | A | 51 | 60.231 | 87.736 | 150.309 | 1.00 | 0.00 | xxxx | 385 |
| ATOM | 386 | C | VAL | A | 51 | 60.984 | 91.419 | 151.177 | 1.00 | 0.00 | xxxx | 386 |
| ATOM | 387 | O | VAL | A | 51 | 61.912 | 91.644 | 151.963 | 1.00 | 0.00 | xxxx | 387 |
| ATOM | 388 | N | ASP | A | 52 | 60.562 | 92.310 | 150.280 | 1.00 | 0.00 | xxxx | 388 |
| ATOM | 389 | CA | ASP | A | 52 | 61.167 | 93.640 | 150.219 | 1.00 | 0.00 | xxxx | 389 |
| ATOM | 390 | CB | ASP | A | 52 | 60.525 | 94.504 | 149.132 | 1.00 | 0.00 | xxxx | 390 |
| ATOM | 391 | CG | ASP | A | 52 | 60.923 | 94.080 | 147.729 | 1.00 | 0.00 | xxxx | 391 |
| ATOM | 392 | OD1 | ASP | A | 52 | 61.889 | 93.306 | 147.580 | 1.00 | 0.00 | xxxx | 392 |
| ATOM | 393 | OD2 | ASP | A | 52 | 60.268 | 94.536 | 146.767 | 1.00 | 0.00 | xxxx | 393 |
| ATOM | 394 | C | ASP | A | 52 | 61.056 | 94.347 | 151.563 | 1.00 | 0.00 | xxxx | 394 |
| ATOM | 395 | O | ASP | A | 52 | 62.012 | 94.987 | 152.006 | 1.00 | 0.00 | xxxx | 395 |
| ATOM | 396 | N | LEU | A | 53 | 59.905 | 94.221 | 152.222 | 1.00 | 0.00 | xxxx | 396 |
| ATOM | 397 | CA | LEU | A | 53 | 59.703 | 94.897 | 153.504 | 1.00 | 0.00 | xxxx | 397 |
| ATOM | 398 | CB | LEU | A | 53 | 58.222 | 94.893 | 153.895 | 1.00 | 0.00 | xxxx | 398 |
| ATOM | 399 | CG | LEU | A | 53 | 57.869 | 95.753 | 155.115 | 1.00 | 0.00 | xxxx | 399 |
| ATOM | 400 | CD1 | LEU | A | 53 | 58.301 | 97.208 | 154.909 | 1.00 | 0.00 | xxxx | 400 |
| ATOM | 401 | CD2 | LEU | A | 53 | 56.381 | 95.663 | 155.432 | 1.00 | 0.00 | xxxx | 401 |
| ATOM | 402 | C | LEU | A | 53 | 60.547 | 94.264 | 154.619 | 1.00 | 0.00 | xxxx | 402 |
| ATOM | 403 | O | LEU | A | 53 | 61.093 | 94.987 | 155.466 | 1.00 | 0.00 | xxxx | 403 |
| ATOM | 404 | N | PHE | A | 54 | 60.636 | 92.933 | 154.629 | 1.00 | 0.00 | xxxx | 404 |
| ATOM | 405 | CA | PHE | A | 54 | 61.515 | 92.214 | 155.560 | 1.00 | 0.00 | xxxx | 405 |
| ATOM | 406 | CB | PHE | A | 54 | 61.561 | 90.708 | 155.254 | 1.00 | 0.00 | xxxx | 406 |
| ATOM | 407 | CG | PHE | A | 54 | 60.346 | 89.933 | 155.713 | 1.00 | 0.00 | xxxx | 407 |
| ATOM | 408 | CD1 | PHE | A | 54 | 59.575 | 90.367 | 156.776 | 1.00 | 0.00 | xxxx | 408 |
| ATOM | 409 | CE1 | PHE | A | 54 | 58.461 | 89.633 | 157.192 | 1.00 | 0.00 | xxxx | 409 |
| ATOM | 410 | CZ | PHE | A | 54 | 58.131 | 88.452 | 156.551 | 1.00 | 0.00 | xxxx | 410 |
| ATOM | 411 | CE2 | PHE | A | 54 | 58.905 | 87.998 | 155.491 | 1.00 | 0.00 | xxxx | 411 |
| ATOM | 412 | CD2 | PHE | A | 54 | 60.010 | 88.737 | 155.089 | 1.00 | 0.00 | xxxx | 412 |
| ATOM | 413 | C | PHE | A | 54 | 62.929 | 92.783 | 155.465 | 1.00 | 0.00 | xxxx | 413 |
| ATOM | 414 | O | PHE | A | 54 | 63.604 | 93.023 | 156.477 | 1.00 | 0.00 | xxxx | 414 |
| ATOM | 415 | N | ILE | A | 55 | 63.373 | 93.007 | 154.233 | 1.00 | 0.00 | xxxx | 415 |
| ATOM | 416 | CA | ILE | A | 55 | 64.732 | 93.479 | 153.993 | 1.00 | 0.00 | xxxx | 416 |
| ATOM | 417 | CB | ILE | A | 55 | 65.098 | 93.302 | 152.511 | 1.00 | 0.00 | xxxx | 417 |
| ATOM | 418 | CG1 | ILE | A | 55 | 65.222 | 91.807 | 152.197 | 1.00 | 0.00 | xxxx | 418 |
| ATOM | 419 | CD1 | ILE | A | 55 | 65.409 | 91.487 | 150.726 | 1.00 | 0.00 | xxxx | 419 |
| ATOM | 420 | CG2 | ILE | A | 55 | 66.377 | 94.055 | 152.177 | 1.00 | 0.00 | xxxx | 420 |
| ATOM | 421 | C | ILE | A | 55 | 64.902 | 94.924 | 154.457 | 1.00 | 0.00 | xxxx | 421 |
| ATOM | 422 | O | ILE | A | 55 | 65.902 | 95.266 | 155.093 | 1.00 | 0.00 | xxxx | 422 |
| ATOM | 423 | N | THR | A | 56 | 63.916 | 95.763 | 154.159 | 1.00 | 0.00 | xxxx | 423 |
| ATOM | 424 | CA | THR | A | 56 | 63.933 | 97.154 | 154.592 | 1.00 | 0.00 | xxxx | 424 |
| ATOM | 425 | CB | THR | A | 56 | 62.713 | 97.923 | 154.040 | 1.00 | 0.00 | xxxx | 425 |
| ATOM | 426 | OG1 | THR | A | 56 | 62.796 | 97.967 | 152.611 | 1.00 | 0.00 | xxxx | 426 |
| ATOM | 427 | CG2 | THR | A | 56 | 62.676 | 99.344 | 154.581 | 1.00 | 0.00 | xxxx | 427 |
| ATOM | 428 | C | THR | A | 56 | 63.951 | 97.232 | 156.116 | 1.00 | 0.00 | xxxx | 428 |
| ATOM | 429 | O | THR | A | 56 | 64.620 | 98.091 | 156.700 | 1.00 | 0.00 | xxxx | 429 |
| ATOM | 430 | N | LYS | A | 57 | 63.230 | 96.315 | 156.755 | 1.00 | 0.00 | xxxx | 430 |
| ATOM | 431 | CA | LYS | A | 57 | 63.159 | 96.274 | 158.214 | 1.00 | 0.00 | xxxx | 431 |
| ATOM | 432 | CB | LYS | A | 57 | 61.850 | 95.617 | 158.655 | 1.00 | 0.00 | xxxx | 432 |
| ATOM | 433 | CG | LYS | A | 57 | 60.626 | 96.490 | 158.452 | 1.00 | 0.00 | xxxx | 433 |
| ATOM | 434 | CD | LYS | A | 57 | 59.376 | 95.824 | 159.010 | 1.00 | 0.00 | xxxx | 434 |
| ATOM | 435 | CE | LYS | A | 57 | 58.180 | 96.769 | 158.976 | 1.00 | 0.00 | xxxx | 435 |
| ATOM | 436 | NZ | LYS | A | 57 | 56.935 | 96.105 | 159.456 | 1.00 | 0.00 | xxxx | 436 |
| ATOM | 437 | C | LYS | A | 57 | 64.344 | 95.549 | 158.864 | 1.00 | 0.00 | xxxx | 437 |
| ATOM | 438 | O | LYS | A | 57 | 64.375 | 95.392 | 160.084 | 1.00 | 0.00 | xxxx | 438 |
| ATOM | 439 | N | LYS | A | 58 | 65.309 | 95.129 | 158.048 | 1.00 | 0.00 | xxxx | 439 |
| ATOM | 440 | CA | LYS | A | 58 | 66.553 | 94.503 | 158.516 | 1.00 | 0.00 | xxxx | 440 |
| ATOM | 441 | CB | LYS | A | 58 | 67.433 | 95.534 | 159.234 | 1.00 | 0.00 | xxxx | 441 |
| ATOM | 442 | CG | LYS | A | 58 | 67.875 | 96.673 | 158.318 | 1.00 | 0.00 | xxxx | 442 |
| ATOM | 443 | CD | LYS | A | 58 | 68.881 | 97.591 | 158.989 | 1.00 | 0.00 | xxxx | 443 |
| ATOM | 444 | CE | LYS | A | 58 | 69.136 | 98.827 | 158.137 | 1.00 | 0.00 | xxxx | 444 |
| ATOM | 445 | NZ | LYS | A | 58 | 69.258 | 98.481 | 156.688 | 1.00 | 0.00 | xxxx | 445 |
| ATOM | 446 | C | LYS | A | 58 | 66.313 | 93.289 | 159.416 | 1.00 | 0.00 | xxxx | 446 |
| ATOM | 447 | O | LYS | A | 58 | 66.865 | 93.191 | 160.516 | 1.00 | 0.00 | xxxx | 447 |
| ATOM | 448 | N | MET | A | 59 | 65.483 | 92.368 | 158.939 | 1.00 | 0.00 | xxxx | 448 |
| ATOM | 449 | CA | MET | A | 59 | 65.349 | 91.068 | 159.584 | 1.00 | 0.00 | xxxx | 449 |
| ATOM | 450 | CB | MET | A | 59 | 64.430 | 90.136 | 158.784 | 1.00 | 0.00 | xxxx | 450 |
| ATOM | 451 | CG | MET | A | 59 | 62.963 | 90.542 | 158.691 | 1.00 | 0.00 | xxxx | 451 |
| ATOM | 452 | SD | MET | A | 59 | 62.059 | 90.456 | 160.248 | 1.00 | 0.00 | xxxx | 452 |
| ATOM | 453 | CE | MET | A | 59 | 62.015 | 92.191 | 160.689 | 1.00 | 0.00 | xxxx | 453 |
| ATOM | 454 | C | MET | A | 59 | 66.728 | 90.444 | 159.704 | 1.00 | 0.00 | xxxx | 454 |
| ATOM | 455 | O | MET | A | 59 | 67.582 | 90.663 | 158.846 | 1.00 | 0.00 | xxxx | 455 |
| ATOM | 456 | N | ASN | A | 60 | 66.951 | 89.661 | 160.756 | 1.00 | 0.00 | xxxx | 456 |
| ATOM | 457 | CA | ASN | A | 60 | 68.228 | 88.965 | 160.908 | 1.00 | 0.00 | xxxx | 457 |
| ATOM | 458 | CB | ASN | A | 60 | 68.446 | 88.589 | 162.372 | 1.00 | 0.00 | xxxx | 458 |
| ATOM | 459 | CG | ASN | A | 60 | 68.464 | 89.801 | 163.279 | 1.00 | 0.00 | xxxx | 459 |
| ATOM | 460 | OD1 | ASN | A | 60 | 67.465 | 90.128 | 163.918 | 1.00 | 0.00 | xxxx | 460 |
| ATOM | 461 | ND2 | ASN | A | 60 | 69.599 | 90.491 | 163.318 | 1.00 | 0.00 | xxxx | 461 |
| ATOM | 462 | C | ASN | A | 60 | 68.341 | 87.719 | 160.026 | 1.00 | 0.00 | xxxx | 462 |
| ATOM | 463 | O | ASN | A | 60 | 69.445 | 87.289 | 159.692 | 1.00 | 0.00 | xxxx | 463 |
| ATOM | 464 | N | ALA | A | 61 | 67.193 | 87.142 | 159.669 | 1.00 | 0.00 | xxxx | 464 |
| ATOM | 465 | CA | ALA | A | 61 | 67.123 | 86.004 | 158.756 | 1.00 | 0.00 | xxxx | 465 |
| ATOM | 466 | CB | ALA | A | 61 | 67.477 | 84.693 | 159.470 | 1.00 | 0.00 | xxxx | 466 |
| ATOM | 467 | C | ALA | A | 61 | 65.727 | 85.920 | 158.155 | 1.00 | 0.00 | xxxx | 467 |
| ATOM | 468 | O | ALA | A | 61 | 64.780 | 86.490 | 158.689 | 1.00 | 0.00 | xxxx | 468 |
| ATOM | 469 | N | LEU | A | 62 | 65.616 | 85.209 | 157.036 | 1.00 | 0.00 | xxxx | 469 |
| ATOM | 470 | CA | LEU | A | 62 | 64.332 | 84.994 | 156.379 | 1.00 | 0.00 | xxxx | 470 |
| ATOM | 471 | CB | LEU | A | 62 | 64.337 | 85.542 | 154.955 | 1.00 | 0.00 | xxxx | 471 |
| ATOM | 472 | CG | LEU | A | 62 | 64.779 | 86.990 | 154.745 | 1.00 | 0.00 | xxxx | 472 |
| ATOM | 473 | CD1 | LEU | A | 62 | 64.760 | 87.308 | 153.264 | 1.00 | 0.00 | xxxx | 473 |
| ATOM | 474 | CD2 | LEU | A | 62 | 63.876 | 87.937 | 155.498 | 1.00 | 0.00 | xxxx | 474 |
| ATOM | 475 | C | LEU | A | 62 | 64.027 | 83.511 | 156.334 | 1.00 | 0.00 | xxxx | 475 |
| ATOM | 476 | O | LEU | A | 62 | 64.923 | 82.701 | 156.109 | 1.00 | 0.00 | xxxx | 476 |
| ATOM | 477 | N | ALA | A | 63 | 62.762 | 83.165 | 156.553 | 1.00 | 0.00 | xxxx | 477 |
| ATOM | 478 | CA | ALA | A | 63 | 62.273 | 81.813 | 156.314 | 1.00 | 0.00 | xxxx | 478 |
| ATOM | 479 | CB | ALA | A | 63 | 61.842 | 81.136 | 157.624 | 1.00 | 0.00 | xxxx | 479 |
| ATOM | 480 | C | ALA | A | 63 | 61.105 | 81.935 | 155.348 | 1.00 | 0.00 | xxxx | 480 |
| ATOM | 481 | O | ALA | A | 63 | 60.050 | 82.455 | 155.711 | 1.00 | 0.00 | xxxx | 481 |
| ATOM | 482 | N | ILE | A | 64 | 61.300 | 81.468 | 154.120 | 1.00 | 0.00 | xxxx | 482 |
| ATOM | 483 | CA | ILE | A | 64 | 60.337 | 81.715 | 153.050 | 1.00 | 0.00 | xxxx | 483 |
| ATOM | 484 | CB | ILE | A | 64 | 60.994 | 82.522 | 151.905 | 1.00 | 0.00 | xxxx | 484 |
| ATOM | 485 | CG1 | ILE | A | 64 | 61.607 | 83.830 | 152.432 | 1.00 | 0.00 | xxxx | 485 |
| ATOM | 486 | CD1 | ILE | A | 64 | 60.611 | 84.779 | 153.082 | 1.00 | 0.00 | xxxx | 486 |
| ATOM | 487 | CG2 | ILE | A | 64 | 59.989 | 82.759 | 150.772 | 1.00 | 0.00 | xxxx | 487 |
| ATOM | 488 | C | ILE | A | 64 | 59.743 | 80.418 | 152.501 | 1.00 | 0.00 | xxxx | 488 |
| ATOM | 489 | O | ILE | A | 64 | 60.463 | 79.495 | 152.140 | 1.00 | 0.00 | xxxx | 489 |
| ATOM | 490 | N | ASN | A | 65 | 58.414 | 80.373 | 152.463 | 1.00 | 0.00 | xxxx | 490 |
| ATOM | 491 | CA | ASN | A | 65 | 57.649 | 79.299 | 151.833 | 1.00 | 0.00 | xxxx | 491 |
| ATOM | 492 | CB | ASN | A | 65 | 56.519 | 78.881 | 152.806 | 1.00 | 0.00 | xxxx | 492 |
| ATOM | 493 | CG | ASN | A | 65 | 55.548 | 77.840 | 152.252 | 1.00 | 0.00 | xxxx | 493 |
| ATOM | 494 | OD1 | ASN | A | 65 | 54.331 | 78.040 | 152.335 | 1.00 | 0.00 | xxxx | 494 |
| ATOM | 495 | ND2 | ASN | A | 65 | 56.055 | 76.722 | 151.762 | 1.00 | 0.00 | xxxx | 495 |
| ATOM | 496 | C | ASN | A | 65 | 57.157 | 79.892 | 150.497 | 1.00 | 0.00 | xxxx | 496 |
| ATOM | 497 | O | ASN | A | 65 | 56.116 | 80.535 | 150.451 | 1.00 | 0.00 | xxxx | 497 |
| ATOM | 498 | N | PRO | A | 66 | 57.946 | 79.739 | 149.413 | 1.00 | 0.00 | xxxx | 498 |
| ATOM | 499 | CA | PRO | A | 66 | 57.699 | 80.580 | 148.231 | 1.00 | 0.00 | xxxx | 499 |
| ATOM | 500 | CB | PRO | A | 66 | 58.934 | 80.329 | 147.357 | 1.00 | 0.00 | xxxx | 500 |
| ATOM | 501 | CG | PRO | A | 66 | 59.372 | 78.935 | 147.731 | 1.00 | 0.00 | xxxx | 501 |
| ATOM | 502 | CD | PRO | A | 66 | 59.106 | 78.847 | 149.232 | 1.00 | 0.00 | xxxx | 502 |
| ATOM | 503 | C | PRO | A | 66 | 56.425 | 80.206 | 147.478 | 1.00 | 0.00 | xxxx | 503 |
| ATOM | 504 | O | PRO | A | 66 | 56.155 | 79.020 | 147.299 | 1.00 | 0.00 | xxxx | 504 |
| ATOM | 505 | N | VAL | A | 67 | 55.650 | 81.199 | 147.049 | 1.00 | 0.00 | xxxx | 505 |
| ATOM | 506 | CA | VAL | A | 67 | 54.412 | 80.915 | 146.326 | 1.00 | 0.00 | xxxx | 506 |
| ATOM | 507 | CB | VAL | A | 67 | 53.585 | 82.188 | 146.099 | 1.00 | 0.00 | xxxx | 507 |
| ATOM | 508 | CG1 | VAL | A | 67 | 52.372 | 81.865 | 145.252 | 1.00 | 0.00 | xxxx | 508 |
| ATOM | 509 | CG2 | VAL | A | 67 | 53.154 | 82.764 | 147.427 | 1.00 | 0.00 | xxxx | 509 |
| ATOM | 510 | C | VAL | A | 67 | 54.743 | 80.241 | 145.004 | 1.00 | 0.00 | xxxx | 510 |
| ATOM | 511 | O | VAL | A | 67 | 54.246 | 79.156 | 144.698 | 1.00 | 0.00 | xxxx | 511 |
| ATOM | 512 | N | ASP | A | 68 | 55.592 | 80.903 | 144.226 | 1.00 | 0.00 | xxxx | 512 |
| ATOM | 513 | CA | ASP | A | 68 | 56.154 | 80.356 | 143.001 | 1.00 | 0.00 | xxxx | 513 |
| ATOM | 514 | CB | ASP | A | 68 | 56.103 | 81.413 | 141.895 | 1.00 | 0.00 | xxxx | 514 |
| ATOM | 515 | CG | ASP | A | 68 | 56.745 | 80.970 | 140.598 | 1.00 | 0.00 | xxxx | 515 |
| ATOM | 516 | OD1 | ASP | A | 68 | 57.339 | 79.872 | 140.524 | 1.00 | 0.00 | xxxx | 516 |
| ATOM | 517 | OD2 | ASP | A | 68 | 56.652 | 81.758 | 139.624 | 1.00 | 0.00 | xxxx | 517 |
| ATOM | 518 | C | ASP | A | 68 | 57.580 | 79.951 | 143.345 | 1.00 | 0.00 | xxxx | 518 |
| ATOM | 519 | O | ASP | A | 68 | 58.439 | 80.805 | 143.546 | 1.00 | 0.00 | xxxx | 519 |
| ATOM | 520 | N | ARG | A | 69 | 57.842 | 78.656 | 143.450 | 1.00 | 0.00 | xxxx | 520 |
| ATOM | 521 | CA | ARG | A | 69 | 59.166 | 78.263 | 143.896 | 1.00 | 0.00 | xxxx | 521 |
| ATOM | 522 | CB | ARG | A | 69 | 59.214 | 76.759 | 144.204 | 1.00 | 0.00 | xxxx | 522 |
| ATOM | 523 | CG | ARG | A | 69 | 59.157 | 75.863 | 142.992 | 1.00 | 0.00 | xxxx | 523 |
| ATOM | 524 | CD | ARG | A | 69 | 58.691 | 74.465 | 143.413 | 1.00 | 0.00 | xxxx | 524 |
| ATOM | 525 | NE | ARG | A | 69 | 58.616 | 73.547 | 142.288 | 1.00 | 0.00 | xxxx | 525 |
| ATOM | 526 | CZ | ARG | A | 69 | 59.624 | 72.786 | 141.865 | 1.00 | 0.00 | xxxx | 526 |
| ATOM | 527 | NH1 | ARG | A | 69 | 60.814 | 72.830 | 142.465 | 1.00 | 0.00 | xxxx | 527 |
| ATOM | 528 | NH2 | ARG | A | 69 | 59.440 | 71.977 | 140.833 | 1.00 | 0.00 | xxxx | 528 |
| ATOM | 529 | C | ARG | A | 69 | 60.249 | 78.666 | 142.881 | 1.00 | 0.00 | xxxx | 529 |
| ATOM | 530 | O | ARG | A | 69 | 61.413 | 78.803 | 143.263 | 1.00 | 0.00 | xxxx | 530 |
| ATOM | 531 | N | THR | A | 70 | 59.879 | 78.923 | 141.625 | 1.00 | 0.00 | xxxx | 531 |
| ATOM | 532 | CA | THR | A | 70 | 60.878 | 79.411 | 140.661 | 1.00 | 0.00 | xxxx | 532 |
| ATOM | 533 | CB | THR | A | 70 | 60.392 | 79.267 | 139.184 | 1.00 | 0.00 | xxxx | 533 |
| ATOM | 534 | OG1 | THR | A | 70 | 59.329 | 80.188 | 138.896 | 1.00 | 0.00 | xxxx | 534 |
| ATOM | 535 | CG2 | THR | A | 70 | 59.919 | 77.842 | 138.933 | 1.00 | 0.00 | xxxx | 535 |
| ATOM | 536 | C | THR | A | 70 | 61.284 | 80.865 | 140.955 | 1.00 | 0.00 | xxxx | 536 |
| ATOM | 537 | O | THR | A | 70 | 62.320 | 81.333 | 140.480 | 1.00 | 0.00 | xxxx | 537 |
| ATOM | 538 | N | ALA | A | 71 | 60.510 | 81.559 | 141.788 | 1.00 | 0.00 | xxxx | 538 |
| ATOM | 539 | CA | ALA | A | 71 | 60.861 | 82.923 | 142.190 | 1.00 | 0.00 | xxxx | 539 |
| ATOM | 540 | CB | ALA | A | 71 | 59.612 | 83.672 | 142.667 | 1.00 | 0.00 | xxxx | 540 |
| ATOM | 541 | C | ALA | A | 71 | 61.937 | 82.971 | 143.274 | 1.00 | 0.00 | xxxx | 541 |
| ATOM | 542 | O | ALA | A | 71 | 62.404 | 84.052 | 143.644 | 1.00 | 0.00 | xxxx | 542 |
| ATOM | 543 | N | ALA | A | 72 | 62.341 | 81.808 | 143.779 | 1.00 | 0.00 | xxxx | 543 |
| ATOM | 544 | CA | ALA | A | 72 | 63.334 | 81.764 | 144.846 | 1.00 | 0.00 | xxxx | 544 |
| ATOM | 545 | CB | ALA | A | 72 | 63.557 | 80.321 | 145.328 | 1.00 | 0.00 | xxxx | 545 |
| ATOM | 546 | C | ALA | A | 72 | 64.654 | 82.393 | 144.397 | 1.00 | 0.00 | xxxx | 546 |
| ATOM | 547 | O | ALA | A | 72 | 65.361 | 82.986 | 145.207 | 1.00 | 0.00 | xxxx | 547 |
| ATOM | 548 | N | GLY | A | 73 | 64.973 | 82.284 | 143.106 | 1.00 | 0.00 | xxxx | 548 |
| ATOM | 549 | CA | GLY | A | 73 | 66.187 | 82.877 | 142.574 | 1.00 | 0.00 | xxxx | 549 |
| ATOM | 550 | C | GLY | A | 73 | 66.250 | 84.379 | 142.794 | 1.00 | 0.00 | xxxx | 550 |
| ATOM | 551 | O | GLY | A | 73 | 67.258 | 84.908 | 143.264 | 1.00 | 0.00 | xxxx | 551 |
| ATOM | 552 | N | THR | A | 74 | 65.167 | 85.071 | 142.457 | 1.00 | 0.00 | xxxx | 552 |
| ATOM | 553 | CA | THR | A | 74 | 65.101 | 86.515 | 142.667 | 1.00 | 0.00 | xxxx | 553 |
| ATOM | 554 | CB | THR | A | 74 | 63.836 | 87.112 | 142.007 | 1.00 | 0.00 | xxxx | 554 |
| ATOM | 555 | OG1 | THR | A | 74 | 63.900 | 86.893 | 140.591 | 1.00 | 0.00 | xxxx | 555 |
| ATOM | 556 | CG2 | THR | A | 74 | 63.743 | 88.608 | 142.273 | 1.00 | 0.00 | xxxx | 556 |
| ATOM | 557 | C | THR | A | 74 | 65.130 | 86.863 | 144.158 | 1.00 | 0.00 | xxxx | 557 |
| ATOM | 558 | O | THR | A | 74 | 65.742 | 87.855 | 144.560 | 1.00 | 0.00 | xxxx | 558 |
| ATOM | 559 | N | ILE | A | 75 | 64.492 | 86.035 | 144.980 | 1.00 | 0.00 | xxxx | 559 |
| ATOM | 560 | CA | ILE | A | 75 | 64.502 | 86.251 | 146.423 | 1.00 | 0.00 | xxxx | 560 |
| ATOM | 561 | CB | ILE | A | 75 | 63.521 | 85.295 | 147.133 | 1.00 | 0.00 | xxxx | 561 |
| ATOM | 562 | CG1 | ILE | A | 75 | 62.077 | 85.609 | 146.730 | 1.00 | 0.00 | xxxx | 562 |
| ATOM | 563 | CD1 | ILE | A | 75 | 61.072 | 84.586 | 147.234 | 1.00 | 0.00 | xxxx | 563 |
| ATOM | 564 | CG2 | ILE | A | 75 | 63.693 | 85.377 | 148.649 | 1.00 | 0.00 | xxxx | 564 |
| ATOM | 565 | C | ILE | A | 75 | 65.924 | 86.099 | 146.973 | 1.00 | 0.00 | xxxx | 565 |
| ATOM | 566 | O | ILE | A | 75 | 66.351 | 86.874 | 147.829 | 1.00 | 0.00 | xxxx | 566 |
| ATOM | 567 | N | ILE | A | 76 | 66.654 | 85.101 | 146.479 | 1.00 | 0.00 | xxxx | 567 |
| ATOM | 568 | CA | ILE | A | 76 | 68.037 | 84.902 | 146.899 | 1.00 | 0.00 | xxxx | 568 |
| ATOM | 569 | CB | ILE | A | 76 | 68.615 | 83.585 | 146.343 | 1.00 | 0.00 | xxxx | 569 |
| ATOM | 570 | CG1 | ILE | A | 76 | 67.917 | 82.387 | 146.999 | 1.00 | 0.00 | xxxx | 570 |
| ATOM | 571 | CD1 | ILE | A | 76 | 68.122 | 81.063 | 146.273 | 1.00 | 0.00 | xxxx | 571 |
| ATOM | 572 | CG2 | ILE | A | 76 | 70.123 | 83.526 | 146.570 | 1.00 | 0.00 | xxxx | 572 |
| ATOM | 573 | C | ILE | A | 76 | 68.884 | 86.109 | 146.491 | 1.00 | 0.00 | xxxx | 573 |
| ATOM | 574 | O | ILE | A | 76 | 69.702 | 86.583 | 147.280 | 1.00 | 0.00 | xxxx | 574 |
| ATOM | 575 | N | ASP | A | 77 | 68.675 | 86.622 | 145.276 | 1.00 | 0.00 | xxxx | 575 |
| ATOM | 576 | CA | ASP | A | 77 | 69.396 | 87.818 | 144.827 | 1.00 | 0.00 | xxxx | 576 |
| ATOM | 577 | CB | ASP | A | 77 | 68.917 | 88.274 | 143.447 | 1.00 | 0.00 | xxxx | 577 |
| ATOM | 578 | CG | ASP | A | 77 | 69.400 | 87.373 | 142.329 | 1.00 | 0.00 | xxxx | 578 |
| ATOM | 579 | OD1 | ASP | A | 77 | 70.394 | 86.651 | 142.530 | 1.00 | 0.00 | xxxx | 579 |
| ATOM | 580 | OD2 | ASP | A | 77 | 68.786 | 87.399 | 141.241 | 1.00 | 0.00 | xxxx | 580 |
| ATOM | 581 | C | ASP | A | 77 | 69.236 | 88.971 | 145.812 | 1.00 | 0.00 | xxxx | 581 |
| ATOM | 582 | O | ASP | A | 77 | 70.217 | 89.600 | 146.202 | 1.00 | 0.00 | xxxx | 582 |
| ATOM | 583 | N | LYS | A | 78 | 67.993 | 89.238 | 146.206 | 1.00 | 0.00 | xxxx | 583 |
| ATOM | 584 | CA | LYS | A | 78 | 67.697 | 90.340 | 147.114 | 1.00 | 0.00 | xxxx | 584 |
| ATOM | 585 | CB | LYS | A | 78 | 66.187 | 90.539 | 147.253 | 1.00 | 0.00 | xxxx | 585 |
| ATOM | 586 | CG | LYS | A | 78 | 65.482 | 91.006 | 145.993 | 1.00 | 0.00 | xxxx | 586 |
| ATOM | 587 | CD | LYS | A | 78 | 63.968 | 90.934 | 146.185 | 1.00 | 0.00 | xxxx | 587 |
| ATOM | 588 | CE | LYS | A | 78 | 63.205 | 91.440 | 144.968 | 1.00 | 0.00 | xxxx | 588 |
| ATOM | 589 | NZ | LYS | A | 78 | 63.228 | 92.922 | 144.844 | 1.00 | 0.00 | xxxx | 589 |
| ATOM | 590 | C | LYS | A | 78 | 68.309 | 90.094 | 148.485 | 1.00 | 0.00 | xxxx | 590 |
| ATOM | 591 | O | LYS | A | 78 | 68.882 | 91.000 | 149.095 | 1.00 | 0.00 | xxxx | 591 |
| ATOM | 592 | N | ALA | A | 79 | 68.177 | 88.864 | 148.962 | 1.00 | 0.00 | xxxx | 592 |
| ATOM | 593 | CA | ALA | A | 79 | 68.713 | 88.492 | 150.266 | 1.00 | 0.00 | xxxx | 593 |
| ATOM | 594 | CB | ALA | A | 79 | 68.272 | 87.087 | 150.631 | 1.00 | 0.00 | xxxx | 594 |
| ATOM | 595 | C | ALA | A | 79 | 70.237 | 88.597 | 150.290 | 1.00 | 0.00 | xxxx | 595 |
| ATOM | 596 | O | ALA | A | 79 | 70.813 | 89.107 | 151.251 | 1.00 | 0.00 | xxxx | 596 |
| ATOM | 597 | N | LYS | A | 80 | 70.885 | 88.117 | 149.231 | 1.00 | 0.00 | xxxx | 597 |
| ATOM | 598 | CA | LYS | A | 80 | 72.346 | 88.192 | 149.124 | 1.00 | 0.00 | xxxx | 598 |
| ATOM | 599 | CB | LYS | A | 80 | 72.829 | 87.505 | 147.843 | 1.00 | 0.00 | xxxx | 599 |
| ATOM | 600 | CG | LYS | A | 80 | 74.345 | 87.412 | 147.695 | 1.00 | 0.00 | xxxx | 600 |
| ATOM | 601 | CD | LYS | A | 80 | 74.716 | 86.576 | 146.474 | 1.00 | 0.00 | xxxx | 601 |
| ATOM | 602 | CE | LYS | A | 80 | 76.222 | 86.416 | 146.331 | 1.00 | 0.00 | xxxx | 602 |
| ATOM | 603 | NZ | LYS | A | 80 | 76.901 | 87.724 | 146.158 | 1.00 | 0.00 | xxxx | 603 |
| ATOM | 604 | C | LYS | A | 80 | 72.823 | 89.638 | 149.149 | 1.00 | 0.00 | xxxx | 604 |
| ATOM | 605 | O | LYS | A | 80 | 73.796 | 89.968 | 149.826 | 1.00 | 0.00 | xxxx | 605 |
| ATOM | 606 | N | GLN | A | 81 | 72.127 | 90.497 | 148.410 | 1.00 | 0.00 | xxxx | 606 |
| ATOM | 607 | CA | GLN | A | 81 | 72.466 | 91.910 | 148.354 | 1.00 | 0.00 | xxxx | 607 |
| ATOM | 608 | CB | GLN | A | 81 | 71.571 | 92.629 | 147.344 | 1.00 | 0.00 | xxxx | 608 |
| ATOM | 609 | CG | GLN | A | 81 | 71.899 | 94.102 | 147.167 | 1.00 | 0.00 | xxxx | 609 |
| ATOM | 610 | CD | GLN | A | 81 | 73.342 | 94.340 | 146.750 | 1.00 | 0.00 | xxxx | 610 |
| ATOM | 611 | OE1 | GLN | A | 81 | 73.913 | 93.578 | 145.966 | 1.00 | 0.00 | xxxx | 611 |
| ATOM | 612 | NE2 | GLN | A | 81 | 73.940 | 95.403 | 147.279 | 1.00 | 0.00 | xxxx | 612 |
| ATOM | 613 | C | GLN | A | 81 | 72.344 | 92.550 | 149.735 | 1.00 | 0.00 | xxxx | 613 |
| ATOM | 614 | O | GLN | A | 81 | 73.172 | 93.369 | 150.123 | 1.00 | 0.00 | xxxx | 614 |
| ATOM | 615 | N | ALA | A | 82 | 71.326 | 92.145 | 150.487 | 1.00 | 0.00 | xxxx | 615 |
| ATOM | 616 | CA | ALA | A | 82 | 71.095 | 92.689 | 151.823 | 1.00 | 0.00 | xxxx | 616 |
| ATOM | 617 | CB | ALA | A | 82 | 69.618 | 92.589 | 152.166 | 1.00 | 0.00 | xxxx | 617 |
| ATOM | 618 | C | ALA | A | 82 | 71.928 | 91.984 | 152.900 | 1.00 | 0.00 | xxxx | 618 |
| ATOM | 619 | O | ALA | A | 82 | 71.942 | 92.409 | 154.058 | 1.00 | 0.00 | xxxx | 619 |
| ATOM | 620 | N | ASN | A | 83 | 72.609 | 90.911 | 152.506 | 1.00 | 0.00 | xxxx | 620 |
| ATOM | 621 | CA | ASN | A | 83 | 73.337 | 90.025 | 153.419 | 1.00 | 0.00 | xxxx | 621 |
| ATOM | 622 | CB | ASN | A | 83 | 74.565 | 90.740 | 154.004 | 1.00 | 0.00 | xxxx | 622 |
| ATOM | 623 | CG | ASN | A | 83 | 75.580 | 89.772 | 154.579 | 1.00 | 0.00 | xxxx | 623 |
| ATOM | 624 | OD1 | ASN | A | 83 | 75.617 | 88.597 | 154.209 | 1.00 | 0.00 | xxxx | 624 |
| ATOM | 625 | ND2 | ASN | A | 83 | 76.400 | 90.256 | 155.498 | 1.00 | 0.00 | xxxx | 625 |
| ATOM | 626 | C | ASN | A | 83 | 72.436 | 89.487 | 154.540 | 1.00 | 0.00 | xxxx | 626 |
| ATOM | 627 | O | ASN | A | 83 | 72.835 | 89.408 | 155.699 | 1.00 | 0.00 | xxxx | 627 |
| ATOM | 628 | N | ILE | A | 84 | 71.215 | 89.108 | 154.173 | 1.00 | 0.00 | xxxx | 628 |
| ATOM | 629 | CA | ILE | A | 84 | 70.283 | 88.471 | 155.098 | 1.00 | 0.00 | xxxx | 629 |
| ATOM | 630 | CB | ILE | A | 84 | 68.926 | 89.201 | 155.137 | 1.00 | 0.00 | xxxx | 630 |
| ATOM | 631 | CG1 | ILE | A | 84 | 69.110 | 90.662 | 155.557 | 1.00 | 0.00 | xxxx | 631 |
| ATOM | 632 | CD1 | ILE | A | 84 | 67.799 | 91.458 | 155.568 | 1.00 | 0.00 | xxxx | 632 |
| ATOM | 633 | CG2 | ILE | A | 84 | 67.970 | 88.472 | 156.085 | 1.00 | 0.00 | xxxx | 633 |
| ATOM | 634 | C | ILE | A | 84 | 70.096 | 87.022 | 154.673 | 1.00 | 0.00 | xxxx | 634 |
| ATOM | 635 | O | ILE | A | 84 | 69.549 | 86.760 | 153.605 | 1.00 | 0.00 | xxxx | 635 |
| ATOM | 636 | N | PRO | A | 85 | 70.566 | 86.072 | 155.494 | 1.00 | 0.00 | xxxx | 636 |
| ATOM | 637 | CA | PRO | A | 85 | 70.477 | 84.653 | 155.134 | 1.00 | 0.00 | xxxx | 637 |
| ATOM | 638 | CB | PRO | A | 85 | 71.146 | 83.939 | 156.313 | 1.00 | 0.00 | xxxx | 638 |
| ATOM | 639 | CG | PRO | A | 85 | 71.926 | 84.981 | 157.018 | 1.00 | 0.00 | xxxx | 639 |
| ATOM | 640 | CD | PRO | A | 85 | 71.196 | 86.272 | 156.807 | 1.00 | 0.00 | xxxx | 640 |
| ATOM | 641 | C | PRO | A | 85 | 69.036 | 84.200 | 154.989 | 1.00 | 0.00 | xxxx | 641 |
| ATOM | 642 | O | PRO | A | 85 | 68.147 | 84.804 | 155.588 | 1.00 | 0.00 | xxxx | 642 |
| ATOM | 643 | N | VAL | A | 86 | 68.805 | 83.152 | 154.208 | 1.00 | 0.00 | xxxx | 643 |
| ATOM | 644 | CA | VAL | A | 86 | 67.445 | 82.710 | 153.973 | 1.00 | 0.00 | xxxx | 644 |
| ATOM | 645 | CB | VAL | A | 86 | 66.891 | 83.341 | 152.664 | 1.00 | 0.00 | xxxx | 645 |
| ATOM | 646 | CG1 | VAL | A | 86 | 67.839 | 83.113 | 151.486 | 1.00 | 0.00 | xxxx | 646 |
| ATOM | 647 | CG2 | VAL | A | 86 | 65.501 | 82.813 | 152.349 | 1.00 | 0.00 | xxxx | 647 |
| ATOM | 648 | C | VAL | A | 86 | 67.355 | 81.197 | 153.939 | 1.00 | 0.00 | xxxx | 648 |
| ATOM | 649 | O | VAL | A | 86 | 68.173 | 80.523 | 153.316 | 1.00 | 0.00 | xxxx | 649 |
| ATOM | 650 | N | VAL | A | 87 | 66.358 | 80.670 | 154.637 | 1.00 | 0.00 | xxxx | 650 |
| ATOM | 651 | CA | VAL | A | 87 | 66.011 | 79.268 | 154.517 | 1.00 | 0.00 | xxxx | 651 |
| ATOM | 652 | CB | VAL | A | 87 | 66.050 | 78.545 | 155.891 | 1.00 | 0.00 | xxxx | 652 |
| ATOM | 653 | CG1 | VAL | A | 87 | 65.197 | 79.281 | 156.950 | 1.00 | 0.00 | xxxx | 653 |
| ATOM | 654 | CG2 | VAL | A | 87 | 65.647 | 77.059 | 155.757 | 1.00 | 0.00 | xxxx | 654 |
| ATOM | 655 | C | VAL | A | 87 | 64.642 | 79.193 | 153.854 | 1.00 | 0.00 | xxxx | 655 |
| ATOM | 656 | O | VAL | A | 87 | 63.702 | 79.888 | 154.250 | 1.00 | 0.00 | xxxx | 656 |
| ATOM | 657 | N | PHE | A | 88 | 64.545 | 78.376 | 152.815 | 1.00 | 0.00 | xxxx | 657 |
| ATOM | 658 | CA | PHE | A | 88 | 63.271 | 78.124 | 152.168 | 1.00 | 0.00 | xxxx | 658 |
| ATOM | 659 | CB | PHE | A | 88 | 63.426 | 78.003 | 150.652 | 1.00 | 0.00 | xxxx | 659 |
| ATOM | 660 | CG | PHE | A | 88 | 63.847 | 79.285 | 149.980 | 1.00 | 0.00 | xxxx | 660 |
| ATOM | 661 | CD1 | PHE | A | 88 | 65.194 | 79.564 | 149.773 | 1.00 | 0.00 | xxxx | 661 |
| ATOM | 662 | CE1 | PHE | A | 88 | 65.594 | 80.743 | 149.161 | 1.00 | 0.00 | xxxx | 662 |
| ATOM | 663 | CZ | PHE | A | 88 | 64.633 | 81.661 | 148.744 | 1.00 | 0.00 | xxxx | 663 |
| ATOM | 664 | CE2 | PHE | A | 88 | 63.286 | 81.391 | 148.944 | 1.00 | 0.00 | xxxx | 664 |
| ATOM | 665 | CD2 | PHE | A | 88 | 62.900 | 80.209 | 149.564 | 1.00 | 0.00 | xxxx | 665 |
| ATOM | 666 | C | PHE | A | 88 | 62.705 | 76.841 | 152.739 | 1.00 | 0.00 | xxxx | 666 |
| ATOM | 667 | O | PHE | A | 88 | 63.451 | 76.004 | 153.249 | 1.00 | 0.00 | xxxx | 667 |
| ATOM | 668 | N | PHE | A | 89 | 61.393 | 76.672 | 152.658 | 1.00 | 0.00 | xxxx | 668 |
| ATOM | 669 | CA | PHE | A | 89 | 60.818 | 75.423 | 153.159 | 1.00 | 0.00 | xxxx | 669 |
| ATOM | 670 | CB | PHE | A | 89 | 60.593 | 75.478 | 154.697 | 1.00 | 0.00 | xxxx | 670 |
| ATOM | 671 | CG | PHE | A | 89 | 59.650 | 76.566 | 155.184 | 1.00 | 0.00 | xxxx | 671 |
| ATOM | 672 | CD1 | PHE | A | 89 | 60.063 | 77.896 | 155.266 | 1.00 | 0.00 | xxxx | 672 |
| ATOM | 673 | CE1 | PHE | A | 89 | 59.200 | 78.894 | 155.744 | 1.00 | 0.00 | xxxx | 673 |
| ATOM | 674 | CZ | PHE | A | 89 | 57.931 | 78.557 | 156.176 | 1.00 | 0.00 | xxxx | 674 |
| ATOM | 675 | CE2 | PHE | A | 89 | 57.517 | 77.232 | 156.118 | 1.00 | 0.00 | xxxx | 675 |
| ATOM | 676 | CD2 | PHE | A | 89 | 58.373 | 76.246 | 155.627 | 1.00 | 0.00 | xxxx | 676 |
| ATOM | 677 | C | PHE | A | 89 | 59.535 | 75.069 | 152.426 | 1.00 | 0.00 | xxxx | 677 |
| ATOM | 678 | O | PHE | A | 89 | 58.814 | 75.946 | 151.946 | 1.00 | 0.00 | xxxx | 678 |
| ATOM | 679 | N | ASN | A | 90 | 59.332 | 73.754 | 152.289 | 1.00 | 0.00 | xxxx | 679 |
| ATOM | 680 | CA | ASN | A | 90 | 58.098 | 73.099 | 151.807 | 1.00 | 0.00 | xxxx | 680 |
| ATOM | 681 | CB | ASN | A | 90 | 56.883 | 73.575 | 152.622 | 1.00 | 0.00 | xxxx | 681 |
| ATOM | 682 | CG | ASN | A | 90 | 55.556 | 73.178 | 151.972 | 1.00 | 0.00 | xxxx | 682 |
| ATOM | 683 | OD1 | ASN | A | 90 | 54.938 | 73.975 | 151.266 | 1.00 | 0.00 | xxxx | 683 |
| ATOM | 684 | ND2 | ASN | A | 90 | 55.117 | 71.939 | 152.207 | 1.00 | 0.00 | xxxx | 684 |
| ATOM | 685 | C | ASN | A | 90 | 57.807 | 73.246 | 150.302 | 1.00 | 0.00 | xxxx | 685 |
| ATOM | 686 | O | ASN | A | 90 | 57.298 | 72.315 | 149.681 | 1.00 | 0.00 | xxxx | 686 |
| ATOM | 687 | N | ARG | A | 91 | 58.125 | 74.403 | 149.724 | 1.00 | 0.00 | xxxx | 687 |
| ATOM | 688 | CA | ARG | A | 91 | 58.036 | 74.592 | 148.280 | 1.00 | 0.00 | xxxx | 688 |
| ATOM | 689 | C | ARG | A | 91 | 59.468 | 74.816 | 147.794 | 1.00 | 0.00 | xxxx | 689 |
| ATOM | 690 | O | ARG | A | 91 | 60.124 | 75.773 | 148.202 | 1.00 | 0.00 | xxxx | 690 |
| ATOM | 691 | CB | ARG | A | 91 | 57.087 | 75.751 | 147.949 | 1.00 | 0.00 | xxxx | 691 |
| ATOM | 692 | CG | ARG | A | 91 | 55.655 | 75.433 | 148.407 | 1.00 | 0.00 | xxxx | 692 |
| ATOM | 693 | CD | ARG | A | 91 | 54.789 | 76.659 | 148.670 | 1.00 | 0.00 | xxxx | 693 |
| ATOM | 694 | NE | ARG | A | 91 | 54.151 | 77.190 | 147.458 | 1.00 | 0.00 | xxxx | 694 |
| ATOM | 695 | CZ | ARG | A | 91 | 53.015 | 76.739 | 146.941 | 1.00 | 0.00 | xxxx | 695 |
| ATOM | 696 | NH1 | ARG | A | 91 | 52.362 | 75.719 | 147.501 | 1.00 | 0.00 | xxxx | 696 |
| ATOM | 697 | NH2 | ARG | A | 91 | 52.519 | 77.309 | 145.846 | 1.00 | 0.00 | xxxx | 697 |
| ATOM | 698 | N | GLU | A | 92 | 59.968 | 73.890 | 146.977 | 1.00 | 0.00 | xxxx | 698 |
| ATOM | 699 | CA | GLU | A | 92 | 61.422 | 73.749 | 146.804 | 1.00 | 0.00 | xxxx | 699 |
| ATOM | 700 | CB | GLU | A | 92 | 61.763 | 72.272 | 146.560 | 1.00 | 0.00 | xxxx | 700 |
| ATOM | 701 | CG | GLU | A | 92 | 63.259 | 71.978 | 146.579 | 1.00 | 0.00 | xxxx | 701 |
| ATOM | 702 | CD | GLU | A | 92 | 63.581 | 70.526 | 146.255 | 1.00 | 0.00 | xxxx | 702 |
| ATOM | 703 | OE1 | GLU | A | 92 | 62.669 | 69.664 | 146.317 | 1.00 | 0.00 | xxxx | 703 |
| ATOM | 704 | OE2 | GLU | A | 92 | 64.758 | 70.244 | 145.929 | 1.00 | 0.00 | xxxx | 704 |
| ATOM | 705 | C | GLU | A | 92 | 62.045 | 74.601 | 145.689 | 1.00 | 0.00 | xxxx | 705 |
| ATOM | 706 | O | GLU | A | 92 | 61.686 | 74.480 | 144.528 | 1.00 | 0.00 | xxxx | 706 |
| ATOM | 707 | N | PRO | A | 93 | 63.012 | 75.455 | 146.038 | 1.00 | 0.00 | xxxx | 707 |
| ATOM | 708 | CA | PRO | A | 93 | 63.750 | 76.176 | 144.988 | 1.00 | 0.00 | xxxx | 708 |
| ATOM | 709 | CB | PRO | A | 93 | 64.800 | 76.963 | 145.766 | 1.00 | 0.00 | xxxx | 709 |
| ATOM | 710 | CG | PRO | A | 93 | 64.178 | 77.136 | 147.143 | 1.00 | 0.00 | xxxx | 710 |
| ATOM | 711 | CD | PRO | A | 93 | 63.424 | 75.864 | 147.392 | 1.00 | 0.00 | xxxx | 711 |
| ATOM | 712 | C | PRO | A | 93 | 64.435 | 75.237 | 144.017 | 1.00 | 0.00 | xxxx | 712 |
| ATOM | 713 | O | PRO | A | 93 | 64.810 | 74.123 | 144.393 | 1.00 | 0.00 | xxxx | 713 |
| ATOM | 714 | N | LEU | A | 94 | 64.606 | 75.690 | 142.781 | 1.00 | 0.00 | xxxx | 714 |
| ATOM | 715 | CA | LEU | A | 94 | 65.328 | 74.912 | 141.787 | 1.00 | 0.00 | xxxx | 715 |
| ATOM | 716 | CB | LEU | A | 94 | 65.233 | 75.591 | 140.425 | 1.00 | 0.00 | xxxx | 716 |
| ATOM | 717 | CG | LEU | A | 94 | 63.785 | 75.791 | 139.968 | 1.00 | 0.00 | xxxx | 717 |
| ATOM | 718 | CD1 | LEU | A | 94 | 63.734 | 76.670 | 138.728 | 1.00 | 0.00 | xxxx | 718 |
| ATOM | 719 | CD2 | LEU | A | 94 | 63.095 | 74.455 | 139.709 | 1.00 | 0.00 | xxxx | 719 |
| ATOM | 720 | C | LEU | A | 94 | 66.781 | 74.727 | 142.223 | 1.00 | 0.00 | xxxx | 720 |
| ATOM | 721 | O | LEU | A | 94 | 67.381 | 75.624 | 142.813 | 1.00 | 0.00 | xxxx | 721 |
| ATOM | 722 | N | PRO | A | 95 | 67.340 | 73.542 | 141.956 | 1.00 | 0.00 | xxxx | 722 |
| ATOM | 723 | CA | PRO | A | 95 | 68.666 | 73.193 | 142.475 | 1.00 | 0.00 | xxxx | 723 |
| ATOM | 724 | CB | PRO | A | 95 | 68.930 | 71.809 | 141.863 | 1.00 | 0.00 | xxxx | 724 |
| ATOM | 725 | CG | PRO | A | 95 | 67.860 | 71.592 | 140.834 | 1.00 | 0.00 | xxxx | 725 |
| ATOM | 726 | CD | PRO | A | 95 | 66.697 | 72.411 | 141.269 | 1.00 | 0.00 | xxxx | 726 |
| ATOM | 727 | C | PRO | A | 95 | 69.755 | 74.205 | 142.096 | 1.00 | 0.00 | xxxx | 727 |
| ATOM | 728 | O | PRO | A | 95 | 70.580 | 74.519 | 142.943 | 1.00 | 0.00 | xxxx | 728 |
| ATOM | 729 | N | GLU | A | 96 | 69.737 | 74.741 | 140.877 | 1.00 | 0.00 | xxxx | 729 |
| ATOM | 730 | CA | GLU | A | 96 | 70.756 | 75.714 | 140.488 | 1.00 | 0.00 | xxxx | 730 |
| ATOM | 731 | CB | GLU | A | 96 | 70.749 | 75.933 | 138.974 | 1.00 | 0.00 | xxxx | 731 |
| ATOM | 732 | CG | GLU | A | 96 | 71.164 | 74.707 | 138.180 | 1.00 | 0.00 | xxxx | 732 |
| ATOM | 733 | CD | GLU | A | 96 | 72.488 | 74.139 | 138.652 | 1.00 | 0.00 | xxxx | 733 |
| ATOM | 734 | OE1 | GLU | A | 96 | 73.461 | 74.918 | 138.775 | 1.00 | 0.00 | xxxx | 734 |
| ATOM | 735 | OE2 | GLU | A | 96 | 72.550 | 72.919 | 138.920 | 1.00 | 0.00 | xxxx | 735 |
| ATOM | 736 | C | GLU | A | 96 | 70.574 | 77.050 | 141.213 | 1.00 | 0.00 | xxxx | 736 |
| ATOM | 737 | O | GLU | A | 96 | 71.546 | 77.766 | 141.459 | 1.00 | 0.00 | xxxx | 737 |
| ATOM | 738 | N | ASP | A | 97 | 69.336 | 77.386 | 141.566 | 1.00 | 0.00 | xxxx | 738 |
| ATOM | 739 | CA | ASP | A | 97 | 69.094 | 78.607 | 142.328 | 1.00 | 0.00 | xxxx | 739 |
| ATOM | 740 | CB | ASP | A | 97 | 67.596 | 78.919 | 142.410 | 1.00 | 0.00 | xxxx | 740 |
| ATOM | 741 | CG | ASP | A | 97 | 67.067 | 79.559 | 141.140 | 1.00 | 0.00 | xxxx | 741 |
| ATOM | 742 | OD1 | ASP | A | 97 | 67.891 | 79.972 | 140.292 | 1.00 | 0.00 | xxxx | 742 |
| ATOM | 743 | OD2 | ASP | A | 97 | 65.832 | 79.670 | 141.000 | 1.00 | 0.00 | xxxx | 743 |
| ATOM | 744 | C | ASP | A | 97 | 69.694 | 78.493 | 143.725 | 1.00 | 0.00 | xxxx | 744 |
| ATOM | 745 | O | ASP | A | 97 | 70.181 | 79.480 | 144.271 | 1.00 | 0.00 | xxxx | 745 |
| ATOM | 746 | N | MET | A | 98 | 69.684 | 77.279 | 144.278 | 1.00 | 0.00 | xxxx | 746 |
| ATOM | 747 | CA | MET | A | 98 | 70.254 | 77.018 | 145.599 | 1.00 | 0.00 | xxxx | 747 |
| ATOM | 748 | CB | MET | A | 98 | 69.955 | 75.577 | 146.039 | 1.00 | 0.00 | xxxx | 748 |
| ATOM | 749 | CG | MET | A | 98 | 68.467 | 75.284 | 146.309 | 1.00 | 0.00 | xxxx | 749 |
| ATOM | 750 | SD | MET | A | 98 | 67.801 | 76.201 | 147.721 | 1.00 | 0.00 | xxxx | 750 |
| ATOM | 751 | CE | MET | A | 98 | 68.810 | 75.553 | 149.068 | 1.00 | 0.00 | xxxx | 751 |
| ATOM | 752 | C | MET | A | 98 | 71.768 | 77.275 | 145.614 | 1.00 | 0.00 | xxxx | 752 |
| ATOM | 753 | O | MET | A | 98 | 72.340 | 77.557 | 146.664 | 1.00 | 0.00 | xxxx | 753 |
| ATOM | 754 | N | LYS | A | 99 | 72.410 | 77.193 | 144.452 | 1.00 | 0.00 | xxxx | 754 |
| ATOM | 755 | CA | LYS | A | 99 | 73.858 | 77.393 | 144.374 | 1.00 | 0.00 | xxxx | 755 |
| ATOM | 756 | CB | LYS | A | 99 | 74.431 | 76.572 | 143.221 | 1.00 | 0.00 | xxxx | 756 |
| ATOM | 757 | CG | LYS | A | 99 | 74.265 | 75.078 | 143.389 | 1.00 | 0.00 | xxxx | 757 |
| ATOM | 758 | CD | LYS | A | 99 | 74.964 | 74.331 | 142.266 | 1.00 | 0.00 | xxxx | 758 |
| ATOM | 759 | CE | LYS | A | 99 | 74.695 | 72.840 | 142.343 | 1.00 | 0.00 | xxxx | 759 |
| ATOM | 760 | NZ | LYS | A | 99 | 75.280 | 72.127 | 141.177 | 1.00 | 0.00 | xxxx | 760 |
| ATOM | 761 | C | LYS | A | 99 | 74.280 | 78.858 | 144.211 | 1.00 | 0.00 | xxxx | 761 |
| ATOM | 762 | O | LYS | A | 99 | 75.475 | 79.153 | 144.093 | 1.00 | 0.00 | xxxx | 762 |
| ATOM | 763 | N | LYS | A | 100 | 73.312 | 79.774 | 144.212 | 1.00 | 0.00 | xxxx | 763 |
| ATOM | 764 | CA | LYS | A | 100 | 73.601 | 81.195 | 144.001 | 1.00 | 0.00 | xxxx | 764 |
| ATOM | 765 | CB | LYS | A | 100 | 72.302 | 81.956 | 143.700 | 1.00 | 0.00 | xxxx | 765 |
| ATOM | 766 | CG | LYS | A | 100 | 71.752 | 81.702 | 142.304 | 1.00 | 0.00 | xxxx | 766 |
| ATOM | 767 | CD | LYS | A | 100 | 70.279 | 82.067 | 142.192 | 1.00 | 0.00 | xxxx | 767 |
| ATOM | 768 | CE | LYS | A | 100 | 70.069 | 83.561 | 142.148 | 1.00 | 0.00 | xxxx | 768 |
| ATOM | 769 | NZ | LYS | A | 100 | 70.519 | 84.144 | 140.855 | 1.00 | 0.00 | xxxx | 769 |
| ATOM | 770 | C | LYS | A | 100 | 74.313 | 81.856 | 145.186 | 1.00 | 0.00 | xxxx | 770 |
| ATOM | 771 | O | LYS | A | 100 | 74.978 | 82.878 | 145.026 | 1.00 | 0.00 | xxxx | 771 |
| ATOM | 772 | N | TRP | A | 101 | 74.173 | 81.273 | 146.372 | 1.00 | 0.00 | xxxx | 772 |
| ATOM | 773 | CA | TRP | A | 101 | 74.705 | 81.880 | 147.591 | 1.00 | 0.00 | xxxx | 773 |
| ATOM | 774 | CB | TRP | A | 101 | 73.730 | 82.932 | 148.125 | 1.00 | 0.00 | xxxx | 774 |
| ATOM | 775 | CG | TRP | A | 101 | 74.285 | 83.873 | 149.158 | 1.00 | 0.00 | xxxx | 775 |
| ATOM | 776 | CD1 | TRP | A | 101 | 75.590 | 84.247 | 149.326 | 1.00 | 0.00 | xxxx | 776 |
| ATOM | 777 | NE1 | TRP | A | 101 | 75.697 | 85.135 | 150.378 | 1.00 | 0.00 | xxxx | 777 |
| ATOM | 778 | CE2 | TRP | A | 101 | 74.451 | 85.343 | 150.909 | 1.00 | 0.00 | xxxx | 778 |
| ATOM | 779 | CD2 | TRP | A | 101 | 73.536 | 84.567 | 150.165 | 1.00 | 0.00 | xxxx | 779 |
| ATOM | 780 | CE3 | TRP | A | 101 | 72.181 | 84.603 | 150.512 | 1.00 | 0.00 | xxxx | 780 |
| ATOM | 781 | CZ3 | TRP | A | 101 | 71.785 | 85.406 | 151.573 | 1.00 | 0.00 | xxxx | 781 |
| ATOM | 782 | CH2 | TRP | A | 101 | 72.716 | 86.169 | 152.289 | 1.00 | 0.00 | xxxx | 782 |
| ATOM | 783 | CZ2 | TRP | A | 101 | 74.051 | 86.153 | 151.975 | 1.00 | 0.00 | xxxx | 783 |
| ATOM | 784 | C | TRP | A | 101 | 74.948 | 80.804 | 148.638 | 1.00 | 0.00 | xxxx | 784 |
| ATOM | 785 | O | TRP | A | 101 | 74.258 | 79.784 | 148.642 | 1.00 | 0.00 | xxxx | 785 |
| ATOM | 786 | N | ASP | A | 102 | 75.911 | 81.031 | 149.529 | 1.00 | 0.00 | xxxx | 786 |
| ATOM | 787 | CA | ASP | A | 102 | 76.271 | 80.015 | 150.516 | 1.00 | 0.00 | xxxx | 787 |
| ATOM | 788 | CB | ASP | A | 102 | 77.797 | 79.937 | 150.667 | 1.00 | 0.00 | xxxx | 788 |
| ATOM | 789 | CG | ASP | A | 102 | 78.411 | 81.214 | 151.221 | 1.00 | 0.00 | xxxx | 789 |
| ATOM | 790 | OD1 | ASP | A | 102 | 77.731 | 82.261 | 151.270 | 1.00 | 0.00 | xxxx | 790 |
| ATOM | 791 | OD2 | ASP | A | 102 | 79.603 | 81.170 | 151.607 | 1.00 | 0.00 | xxxx | 791 |
| ATOM | 792 | C | ASP | A | 102 | 75.612 | 80.254 | 151.872 | 1.00 | 0.00 | xxxx | 792 |
| ATOM | 793 | O | ASP | A | 102 | 76.036 | 79.692 | 152.882 | 1.00 | 0.00 | xxxx | 793 |
| ATOM | 794 | N | LYS | A | 103 | 74.569 | 81.079 | 151.888 | 1.00 | 0.00 | xxxx | 794 |
| ATOM | 795 | CA | LYS | A | 103 | 73.775 | 81.274 | 153.099 | 1.00 | 0.00 | xxxx | 795 |
| ATOM | 796 | CB | LYS | A | 103 | 73.948 | 82.698 | 153.648 | 1.00 | 0.00 | xxxx | 796 |
| ATOM | 797 | CG | LYS | A | 103 | 75.400 | 83.028 | 154.036 | 1.00 | 0.00 | xxxx | 797 |
| ATOM | 798 | CD | LYS | A | 103 | 75.491 | 84.301 | 154.862 | 1.00 | 0.00 | xxxx | 798 |
| ATOM | 799 | CE | LYS | A | 103 | 76.930 | 84.638 | 155.211 | 1.00 | 0.00 | xxxx | 799 |
| ATOM | 800 | NZ | LYS | A | 103 | 77.020 | 85.940 | 155.922 | 1.00 | 0.00 | xxxx | 800 |
| ATOM | 801 | C | LYS | A | 103 | 72.314 | 80.970 | 152.799 | 1.00 | 0.00 | xxxx | 801 |
| ATOM | 802 | O | LYS | A | 103 | 71.412 | 81.586 | 153.365 | 1.00 | 0.00 | xxxx | 802 |
| ATOM | 803 | N | VAL | A | 104 | 72.094 | 80.004 | 151.909 | 1.00 | 0.00 | xxxx | 803 |
| ATOM | 804 | CA | VAL | A | 104 | 70.753 | 79.586 | 151.511 | 1.00 | 0.00 | xxxx | 804 |
| ATOM | 805 | CB | VAL | A | 104 | 70.521 | 79.835 | 150.010 | 1.00 | 0.00 | xxxx | 805 |
| ATOM | 806 | CG1 | VAL | A | 104 | 69.112 | 79.405 | 149.604 | 1.00 | 0.00 | xxxx | 806 |
| ATOM | 807 | CG2 | VAL | A | 104 | 70.758 | 81.302 | 149.682 | 1.00 | 0.00 | xxxx | 807 |
| ATOM | 808 | C | VAL | A | 104 | 70.528 | 78.111 | 151.844 | 1.00 | 0.00 | xxxx | 808 |
| ATOM | 809 | O | VAL | A | 104 | 71.358 | 77.265 | 151.521 | 1.00 | 0.00 | xxxx | 809 |
| ATOM | 810 | N | TYR | A | 105 | 69.388 | 77.809 | 152.466 | 1.00 | 0.00 | xxxx | 810 |
| ATOM | 811 | CA | TYR | A | 105 | 69.059 | 76.444 | 152.853 | 1.00 | 0.00 | xxxx | 811 |
| ATOM | 812 | CB | TYR | A | 105 | 69.228 | 76.265 | 154.376 | 1.00 | 0.00 | xxxx | 812 |
| ATOM | 813 | CG | TYR | A | 105 | 70.671 | 76.389 | 154.807 | 1.00 | 0.00 | xxxx | 813 |
| ATOM | 814 | CD1 | TYR | A | 105 | 71.253 | 77.636 | 154.992 | 1.00 | 0.00 | xxxx | 814 |
| ATOM | 815 | CE1 | TYR | A | 105 | 72.581 | 77.756 | 155.351 | 1.00 | 0.00 | xxxx | 815 |
| ATOM | 816 | CZ | TYR | A | 105 | 73.346 | 76.619 | 155.527 | 1.00 | 0.00 | xxxx | 816 |
| ATOM | 817 | OH | TYR | A | 105 | 74.672 | 76.740 | 155.883 | 1.00 | 0.00 | xxxx | 817 |
| ATOM | 818 | CE2 | TYR | A | 105 | 72.793 | 75.365 | 155.342 | 1.00 | 0.00 | xxxx | 818 |
| ATOM | 819 | CD2 | TYR | A | 105 | 71.467 | 75.257 | 154.980 | 1.00 | 0.00 | xxxx | 819 |
| ATOM | 820 | C | TYR | A | 105 | 67.638 | 76.081 | 152.436 | 1.00 | 0.00 | xxxx | 820 |
| ATOM | 821 | O | TYR | A | 105 | 66.846 | 76.955 | 152.088 | 1.00 | 0.00 | xxxx | 821 |
| ATOM | 822 | N | TYR | A | 106 | 67.328 | 74.787 | 152.466 | 1.00 | 0.00 | xxxx | 822 |
| ATOM | 823 | CA | TYR | A | 106 | 65.968 | 74.320 | 152.202 | 1.00 | 0.00 | xxxx | 823 |
| ATOM | 824 | CB | TYR | A | 106 | 65.805 | 73.799 | 150.766 | 1.00 | 0.00 | xxxx | 824 |
| ATOM | 825 | CG | TYR | A | 106 | 64.400 | 73.263 | 150.542 | 1.00 | 0.00 | xxxx | 825 |
| ATOM | 826 | CD1 | TYR | A | 106 | 63.320 | 74.136 | 150.424 | 1.00 | 0.00 | xxxx | 826 |
| ATOM | 827 | CE1 | TYR | A | 106 | 62.021 | 73.665 | 150.254 | 1.00 | 0.00 | xxxx | 827 |
| ATOM | 828 | CZ | TYR | A | 106 | 61.779 | 72.307 | 150.206 | 1.00 | 0.00 | xxxx | 828 |
| ATOM | 829 | OH | TYR | A | 106 | 60.486 | 71.885 | 150.034 | 1.00 | 0.00 | xxxx | 829 |
| ATOM | 830 | CE2 | TYR | A | 106 | 62.821 | 71.409 | 150.321 | 1.00 | 0.00 | xxxx | 830 |
| ATOM | 831 | CD2 | TYR | A | 106 | 64.140 | 71.890 | 150.506 | 1.00 | 0.00 | xxxx | 831 |
| ATOM | 832 | C | TYR | A | 106 | 65.611 | 73.206 | 153.170 | 1.00 | 0.00 | xxxx | 832 |
| ATOM | 833 | O | TYR | A | 106 | 66.406 | 72.284 | 153.372 | 1.00 | 0.00 | xxxx | 833 |
| ATOM | 834 | N | VAL | A | 107 | 64.421 | 73.298 | 153.763 | 1.00 | 0.00 | xxxx | 834 |
| ATOM | 835 | CA | VAL | A | 107 | 63.878 | 72.225 | 154.590 | 1.00 | 0.00 | xxxx | 835 |
| ATOM | 836 | CB | VAL | A | 107 | 63.576 | 72.705 | 156.023 | 1.00 | 0.00 | xxxx | 836 |
| ATOM | 837 | CG1 | VAL | A | 107 | 62.977 | 71.559 | 156.841 | 1.00 | 0.00 | xxxx | 837 |
| ATOM | 838 | CG2 | VAL | A | 107 | 64.837 | 73.246 | 156.691 | 1.00 | 0.00 | xxxx | 838 |
| ATOM | 839 | C | VAL | A | 107 | 62.621 | 71.650 | 153.968 | 1.00 | 0.00 | xxxx | 839 |
| ATOM | 840 | O | VAL | A | 107 | 61.683 | 72.388 | 153.638 | 1.00 | 0.00 | xxxx | 840 |
| ATOM | 841 | N | GLY | A | 108 | 62.582 | 70.329 | 153.842 | 1.00 | 0.00 | xxxx | 841 |
| ATOM | 842 | CA | GLY | A | 108 | 61.396 | 69.675 | 153.322 | 1.00 | 0.00 | xxxx | 842 |
| ATOM | 843 | C | GLY | A | 108 | 61.471 | 68.179 | 153.492 | 1.00 | 0.00 | xxxx | 843 |
| ATOM | 844 | O | GLY | A | 108 | 61.937 | 67.673 | 154.513 | 1.00 | 0.00 | xxxx | 844 |
| ATOM | 845 | N | ALA | A | 109 | 61.009 | 67.467 | 152.473 | 1.00 | 0.00 | xxxx | 845 |
| ATOM | 846 | CA | ALA | A | 109 | 60.962 | 66.021 | 152.524 | 1.00 | 0.00 | xxxx | 846 |
| ATOM | 847 | CB | ALA | A | 109 | 59.693 | 65.555 | 153.212 | 1.00 | 0.00 | xxxx | 847 |
| ATOM | 848 | C | ALA | A | 109 | 61.050 | 65.499 | 151.101 | 1.00 | 0.00 | xxxx | 848 |
| ATOM | 849 | O | ALA | A | 109 | 60.772 | 66.233 | 150.154 | 1.00 | 0.00 | xxxx | 849 |
| ATOM | 850 | N | LYS | A | 110 | 61.447 | 64.244 | 150.951 | 1.00 | 0.00 | xxxx | 850 |
| ATOM | 851 | CA | LYS | A | 110 | 61.636 | 63.675 | 149.621 | 1.00 | 0.00 | xxxx | 851 |
| ATOM | 852 | CB | LYS | A | 110 | 62.642 | 62.528 | 149.664 | 1.00 | 0.00 | xxxx | 852 |
| ATOM | 853 | CG | LYS | A | 110 | 64.070 | 62.996 | 149.934 | 1.00 | 0.00 | xxxx | 853 |
| ATOM | 854 | CD | LYS | A | 110 | 64.508 | 63.980 | 148.850 | 1.00 | 0.00 | xxxx | 854 |
| ATOM | 855 | CE | LYS | A | 110 | 66.018 | 64.004 | 148.679 | 1.00 | 0.00 | xxxx | 855 |
| ATOM | 856 | NZ | LYS | A | 110 | 66.526 | 62.763 | 148.039 | 1.00 | 0.00 | xxxx | 856 |
| ATOM | 857 | C | LYS | A | 110 | 60.300 | 63.210 | 149.074 | 1.00 | 0.00 | xxxx | 857 |
| ATOM | 858 | O | LYS | A | 110 | 59.803 | 62.153 | 149.444 | 1.00 | 0.00 | xxxx | 858 |
| ATOM | 859 | N | ALA | A | 111 | 59.726 | 64.006 | 148.173 | 1.00 | 0.00 | xxxx | 859 |
| ATOM | 860 | CA | ALA | A | 111 | 58.378 | 63.731 | 147.687 | 1.00 | 0.00 | xxxx | 860 |
| ATOM | 861 | CB | ALA | A | 111 | 57.933 | 64.817 | 146.714 | 1.00 | 0.00 | xxxx | 861 |
| ATOM | 862 | C | ALA | A | 111 | 58.276 | 62.352 | 147.025 | 1.00 | 0.00 | xxxx | 862 |
| ATOM | 863 | O | ALA | A | 111 | 57.214 | 61.731 | 147.064 | 1.00 | 0.00 | xxxx | 863 |
| ATOM | 864 | N | GLU | A | 112 | 59.371 | 61.894 | 146.409 | 1.00 | 0.00 | xxxx | 864 |
| ATOM | 865 | CA | GLU | A | 112 | 59.416 | 60.558 | 145.815 | 1.00 | 0.00 | xxxx | 865 |
| ATOM | 866 | C | GLU | A | 112 | 59.013 | 59.494 | 146.832 | 1.00 | 0.00 | xxxx | 866 |
| ATOM | 867 | O | GLU | A | 112 | 58.281 | 58.552 | 146.520 | 1.00 | 0.00 | xxxx | 867 |
| ATOM | 868 | CB | GLU | A | 112 | 60.820 | 60.239 | 145.276 | 1.00 | 0.00 | xxxx | 868 |
| ATOM | 869 | CG | GLU | A | 112 | 61.342 | 61.190 | 144.209 | 1.00 | 0.00 | xxxx | 869 |
| ATOM | 870 | CD | GLU | A | 112 | 62.128 | 62.364 | 144.774 | 1.00 | 0.00 | xxxx | 870 |
| ATOM | 871 | OE1 | GLU | A | 112 | 61.879 | 62.772 | 145.933 | 1.00 | 0.00 | xxxx | 871 |
| ATOM | 872 | OE2 | GLU | A | 112 | 63.009 | 62.882 | 144.047 | 1.00 | 0.00 | xxxx | 872 |
| ATOM | 873 | N | GLN | A | 113 | 59.510 | 59.649 | 148.054 | 1.00 | 0.00 | xxxx | 873 |
| ATOM | 874 | C | GLN | A | 113 | 57.749 | 58.688 | 149.484 | 1.00 | 0.00 | xxxx | 874 |
| ATOM | 875 | O | GLN | A | 113 | 57.153 | 57.624 | 149.697 | 1.00 | 0.00 | xxxx | 875 |
| ATOM | 876 | CA | GLN | A | 113 | 59.241 | 58.715 | 149.144 | 1.00 | 0.00 | xxxx | 876 |
| ATOM | 877 | CB | GLN | A | 113 | 60.077 | 59.090 | 150.385 | 1.00 | 0.00 | xxxx | 877 |
| ATOM | 878 | CG | GLN | A | 113 | 59.812 | 58.227 | 151.626 | 1.00 | 0.00 | xxxx | 878 |
| ATOM | 879 | CD | GLN | A | 113 | 60.629 | 58.637 | 152.864 | 1.00 | 0.00 | xxxx | 879 |
| ATOM | 880 | OE1 | GLN | A | 113 | 60.781 | 59.826 | 153.183 | 1.00 | 0.00 | xxxx | 880 |
| ATOM | 881 | NE2 | GLN | A | 113 | 61.153 | 57.640 | 153.567 | 1.00 | 0.00 | xxxx | 881 |
| ATOM | 882 | N | SER | A | 114 | 57.135 | 59.870 | 149.559 | 1.00 | 0.00 | xxxx | 882 |
| ATOM | 883 | CA | SER | A | 114 | 55.708 | 59.885 | 149.877 | 1.00 | 0.00 | xxxx | 883 |
| ATOM | 884 | CB | SER | A | 114 | 55.192 | 61.303 | 150.175 | 1.00 | 0.00 | xxxx | 884 |
| ATOM | 885 | OG | SER | A | 114 | 55.337 | 62.195 | 149.078 | 1.00 | 0.00 | xxxx | 885 |
| ATOM | 886 | C | SER | A | 114 | 54.891 | 59.240 | 148.752 | 1.00 | 0.00 | xxxx | 886 |
| ATOM | 887 | O | SER | A | 114 | 53.912 | 58.554 | 149.022 | 1.00 | 0.00 | xxxx | 887 |
| ATOM | 888 | N | GLY | A | 115 | 55.302 | 59.440 | 147.504 | 1.00 | 0.00 | xxxx | 888 |
| ATOM | 889 | CA | GLY | A | 115 | 54.639 | 58.793 | 146.382 | 1.00 | 0.00 | xxxx | 889 |
| ATOM | 890 | C | GLY | A | 115 | 54.745 | 57.275 | 146.440 | 1.00 | 0.00 | xxxx | 890 |
| ATOM | 891 | O | GLY | A | 115 | 53.756 | 56.555 | 146.241 | 1.00 | 0.00 | xxxx | 891 |
| ATOM | 892 | N | ILE | A | 116 | 55.950 | 56.787 | 146.718 | 1.00 | 0.00 | xxxx | 892 |
| ATOM | 893 | CA | ILE | A | 116 | 56.167 | 55.351 | 146.855 | 1.00 | 0.00 | xxxx | 893 |
| ATOM | 894 | CB | ILE | A | 116 | 57.644 | 55.034 | 147.118 | 1.00 | 0.00 | xxxx | 894 |
| ATOM | 895 | CG1 | ILE | A | 116 | 58.482 | 55.388 | 145.876 | 1.00 | 0.00 | xxxx | 895 |
| ATOM | 896 | CD1 | ILE | A | 116 | 59.970 | 55.429 | 146.116 | 1.00 | 0.00 | xxxx | 896 |
| ATOM | 897 | CG2 | ILE | A | 116 | 57.816 | 53.563 | 147.498 | 1.00 | 0.00 | xxxx | 897 |
| ATOM | 898 | C | ILE | A | 116 | 55.285 | 54.778 | 147.960 | 1.00 | 0.00 | xxxx | 898 |
| ATOM | 899 | O | ILE | A | 116 | 54.631 | 53.756 | 147.766 | 1.00 | 0.00 | xxxx | 899 |
| ATOM | 900 | N | LEU | A | 117 | 55.258 | 55.431 | 149.120 | 1.00 | 0.00 | xxxx | 900 |
| ATOM | 901 | CA | LEU | A | 117 | 54.491 | 54.889 | 150.236 | 1.00 | 0.00 | xxxx | 901 |
| ATOM | 902 | CB | LEU | A | 117 | 54.789 | 55.673 | 151.520 | 1.00 | 0.00 | xxxx | 902 |
| ATOM | 903 | CG | LEU | A | 117 | 56.224 | 55.505 | 152.034 | 1.00 | 0.00 | xxxx | 903 |
| ATOM | 904 | CD1 | LEU | A | 117 | 56.507 | 56.501 | 153.152 | 1.00 | 0.00 | xxxx | 904 |
| ATOM | 905 | CD2 | LEU | A | 117 | 56.470 | 54.082 | 152.518 | 1.00 | 0.00 | xxxx | 905 |
| ATOM | 906 | C | LEU | A | 117 | 52.991 | 54.878 | 149.916 | 1.00 | 0.00 | xxxx | 906 |
| ATOM | 907 | O | LEU | A | 117 | 52.282 | 53.928 | 150.270 | 1.00 | 0.00 | xxxx | 907 |
| ATOM | 908 | N | GLN | A | 118 | 52.514 | 55.931 | 149.255 | 1.00 | 0.00 | xxxx | 908 |
| ATOM | 909 | CA | GLN | A | 118 | 51.141 | 55.976 | 148.746 | 1.00 | 0.00 | xxxx | 909 |
| ATOM | 910 | CB | GLN | A | 118 | 50.901 | 57.223 | 147.902 | 1.00 | 0.00 | xxxx | 910 |
| ATOM | 911 | CG | GLN | A | 118 | 50.449 | 58.426 | 148.646 | 1.00 | 0.00 | xxxx | 911 |
| ATOM | 912 | CD | GLN | A | 118 | 49.879 | 59.451 | 147.701 | 1.00 | 0.00 | xxxx | 912 |
| ATOM | 913 | OE1 | GLN | A | 118 | 50.580 | 59.948 | 146.809 | 1.00 | 0.00 | xxxx | 913 |
| ATOM | 914 | NE2 | GLN | A | 118 | 48.596 | 59.751 | 147.861 | 1.00 | 0.00 | xxxx | 914 |
| ATOM | 915 | C | GLN | A | 118 | 50.837 | 54.774 | 147.884 | 1.00 | 0.00 | xxxx | 915 |
| ATOM | 916 | O | GLN | A | 118 | 49.828 | 54.083 | 148.072 | 1.00 | 0.00 | xxxx | 916 |
| ATOM | 917 | N | GLY | A | 119 | 51.710 | 54.554 | 146.908 | 1.00 | 0.00 | xxxx | 917 |
| ATOM | 918 | CA | GLY | A | 119 | 51.531 | 53.469 | 145.958 | 1.00 | 0.00 | xxxx | 918 |
| ATOM | 919 | C | GLY | A | 119 | 51.496 | 52.113 | 146.637 | 1.00 | 0.00 | xxxx | 919 |
| ATOM | 920 | O | GLY | A | 119 | 50.727 | 51.240 | 146.249 | 1.00 | 0.00 | xxxx | 920 |
| ATOM | 921 | N | GLN | A | 120 | 52.328 | 51.939 | 147.659 | 1.00 | 0.00 | xxxx | 921 |
| ATOM | 922 | CA | GLN | A | 120 | 52.344 | 50.681 | 148.396 | 1.00 | 0.00 | xxxx | 922 |
| ATOM | 923 | CB | GLN | A | 120 | 53.474 | 50.671 | 149.420 | 1.00 | 0.00 | xxxx | 923 |
| ATOM | 924 | CG | GLN | A | 120 | 54.836 | 50.619 | 148.765 | 1.00 | 0.00 | xxxx | 924 |
| ATOM | 925 | CD | GLN | A | 120 | 55.953 | 50.609 | 149.768 | 1.00 | 0.00 | xxxx | 925 |
| ATOM | 926 | OE1 | GLN | A | 120 | 55.725 | 50.749 | 150.967 | 1.00 | 0.00 | xxxx | 926 |
| ATOM | 927 | NE2 | GLN | A | 120 | 57.179 | 50.439 | 149.284 | 1.00 | 0.00 | xxxx | 927 |
| ATOM | 928 | C | GLN | A | 120 | 51.007 | 50.421 | 149.087 | 1.00 | 0.00 | xxxx | 928 |
| ATOM | 929 | O | GLN | A | 120 | 50.520 | 49.297 | 149.080 | 1.00 | 0.00 | xxxx | 929 |
| ATOM | 930 | N | ILE | A | 121 | 50.424 | 51.463 | 149.674 | 1.00 | 0.00 | xxxx | 930 |
| ATOM | 931 | CA | ILE | A | 121 | 49.140 | 51.321 | 150.354 | 1.00 | 0.00 | xxxx | 931 |
| ATOM | 932 | CB | ILE | A | 121 | 48.686 | 52.659 | 150.977 | 1.00 | 0.00 | xxxx | 932 |
| ATOM | 933 | CG1 | ILE | A | 121 | 49.633 | 53.085 | 152.095 | 1.00 | 0.00 | xxxx | 933 |
| ATOM | 934 | CD1 | ILE | A | 121 | 49.463 | 54.550 | 152.512 | 1.00 | 0.00 | xxxx | 934 |
| ATOM | 935 | CG2 | ILE | A | 121 | 47.244 | 52.556 | 151.489 | 1.00 | 0.00 | xxxx | 935 |
| ATOM | 936 | C | ILE | A | 121 | 48.095 | 50.799 | 149.377 | 1.00 | 0.00 | xxxx | 936 |
| ATOM | 937 | O | ILE | A | 121 | 47.365 | 49.831 | 149.663 | 1.00 | 0.00 | xxxx | 937 |
| ATOM | 938 | N | MET | A | 122 | 48.027 | 51.433 | 148.213 | 1.00 | 0.00 | xxxx | 938 |
| ATOM | 939 | CA | MET | A | 122 | 47.014 | 51.056 | 147.233 | 1.00 | 0.00 | xxxx | 939 |
| ATOM | 940 | CB | MET | A | 122 | 46.928 | 52.108 | 146.118 | 1.00 | 0.00 | xxxx | 940 |
| ATOM | 941 | CG | MET | A | 122 | 45.735 | 51.898 | 145.188 | 1.00 | 0.00 | xxxx | 941 |
| ATOM | 942 | SD | MET | A | 122 | 44.169 | 52.297 | 146.002 | 1.00 | 0.00 | xxxx | 942 |
| ATOM | 943 | CE | MET | A | 122 | 44.193 | 54.082 | 145.894 | 1.00 | 0.00 | xxxx | 943 |
| ATOM | 944 | C | MET | A | 122 | 47.289 | 49.677 | 146.640 | 1.00 | 0.00 | xxxx | 944 |
| ATOM | 945 | O | MET | A | 122 | 46.362 | 48.898 | 146.428 | 1.00 | 0.00 | xxxx | 945 |
| ATOM | 946 | N | ALA | A | 123 | 48.557 | 49.374 | 146.366 | 1.00 | 0.00 | xxxx | 946 |
| ATOM | 947 | CA | ALA | A | 123 | 48.899 | 48.085 | 145.771 | 1.00 | 0.00 | xxxx | 947 |
| ATOM | 948 | CB | ALA | A | 123 | 50.363 | 48.040 | 145.366 | 1.00 | 0.00 | xxxx | 948 |
| ATOM | 949 | C | ALA | A | 123 | 48.590 | 46.950 | 146.730 | 1.00 | 0.00 | xxxx | 949 |
| ATOM | 950 | O | ALA | A | 123 | 48.059 | 45.914 | 146.332 | 1.00 | 0.00 | xxxx | 950 |
| ATOM | 951 | N | ASP | A | 124 | 48.920 | 47.150 | 148.000 | 1.00 | 0.00 | xxxx | 951 |
| ATOM | 952 | CA | ASP | A | 124 | 48.670 | 46.114 | 148.993 | 1.00 | 0.00 | xxxx | 952 |
| ATOM | 953 | CB | ASP | A | 124 | 49.330 | 46.479 | 150.319 | 1.00 | 0.00 | xxxx | 953 |
| ATOM | 954 | CG | ASP | A | 124 | 50.841 | 46.290 | 150.288 | 1.00 | 0.00 | xxxx | 954 |
| ATOM | 955 | OD1 | ASP | A | 124 | 51.333 | 45.512 | 149.430 | 1.00 | 0.00 | xxxx | 955 |
| ATOM | 956 | OD2 | ASP | A | 124 | 51.528 | 46.902 | 151.132 | 1.00 | 0.00 | xxxx | 956 |
| ATOM | 957 | C | ASP | A | 124 | 47.170 | 45.881 | 149.179 | 1.00 | 0.00 | xxxx | 957 |
| ATOM | 958 | O | ASP | A | 124 | 46.724 | 44.736 | 149.314 | 1.00 | 0.00 | xxxx | 958 |
| ATOM | 959 | N | TYR | A | 125 | 46.388 | 46.960 | 149.168 | 1.00 | 0.00 | xxxx | 959 |
| ATOM | 960 | CA | TYR | A | 125 | 44.936 | 46.818 | 149.209 | 1.00 | 0.00 | xxxx | 960 |
| ATOM | 961 | CB | TYR | A | 125 | 44.248 | 48.197 | 149.260 | 1.00 | 0.00 | xxxx | 961 |
| ATOM | 962 | CG | TYR | A | 125 | 42.757 | 48.059 | 149.036 | 1.00 | 0.00 | xxxx | 962 |
| ATOM | 963 | CD1 | TYR | A | 125 | 41.909 | 47.716 | 150.080 | 1.00 | 0.00 | xxxx | 963 |
| ATOM | 964 | CE1 | TYR | A | 125 | 40.544 | 47.555 | 149.867 | 1.00 | 0.00 | xxxx | 964 |
| ATOM | 965 | CZ | TYR | A | 125 | 40.038 | 47.721 | 148.584 | 1.00 | 0.00 | xxxx | 965 |
| ATOM | 966 | OH | TYR | A | 125 | 38.691 | 47.570 | 148.331 | 1.00 | 0.00 | xxxx | 966 |
| ATOM | 967 | CE2 | TYR | A | 125 | 40.867 | 48.050 | 147.541 | 1.00 | 0.00 | xxxx | 967 |
| ATOM | 968 | CD2 | TYR | A | 125 | 42.214 | 48.216 | 147.768 | 1.00 | 0.00 | xxxx | 968 |
| ATOM | 969 | C | TYR | A | 125 | 44.427 | 46.015 | 147.999 | 1.00 | 0.00 | xxxx | 969 |
| ATOM | 970 | O | TYR | A | 125 | 43.628 | 45.081 | 148.146 | 1.00 | 0.00 | xxxx | 970 |
| ATOM | 971 | N | TRP | A | 126 | 44.895 | 46.387 | 146.810 | 1.00 | 0.00 | xxxx | 971 |
| ATOM | 972 | CA | TRP | A | 126 | 44.453 | 45.762 | 145.565 | 1.00 | 0.00 | xxxx | 972 |
| ATOM | 973 | CB | TRP | A | 126 | 45.160 | 46.427 | 144.377 | 1.00 | 0.00 | xxxx | 973 |
| ATOM | 974 | CG | TRP | A | 126 | 44.797 | 45.873 | 143.033 | 1.00 | 0.00 | xxxx | 974 |
| ATOM | 975 | CD1 | TRP | A | 126 | 45.447 | 44.883 | 142.351 | 1.00 | 0.00 | xxxx | 975 |
| ATOM | 976 | NE1 | TRP | A | 126 | 44.821 | 44.649 | 141.150 | 1.00 | 0.00 | xxxx | 976 |
| ATOM | 977 | CE2 | TRP | A | 126 | 43.755 | 45.503 | 141.032 | 1.00 | 0.00 | xxxx | 977 |
| ATOM | 978 | CD2 | TRP | A | 126 | 43.709 | 46.289 | 142.199 | 1.00 | 0.00 | xxxx | 978 |
| ATOM | 979 | CE3 | TRP | A | 126 | 42.695 | 47.242 | 142.333 | 1.00 | 0.00 | xxxx | 979 |
| ATOM | 980 | CZ3 | TRP | A | 126 | 41.773 | 47.380 | 141.304 | 1.00 | 0.00 | xxxx | 980 |
| ATOM | 981 | CH2 | TRP | A | 126 | 41.849 | 46.584 | 140.159 | 1.00 | 0.00 | xxxx | 981 |
| ATOM | 982 | CZ2 | TRP | A | 126 | 42.829 | 45.642 | 140.001 | 1.00 | 0.00 | xxxx | 982 |
| ATOM | 983 | C | TRP | A | 126 | 44.722 | 44.254 | 145.590 | 1.00 | 0.00 | xxxx | 983 |
| ATOM | 984 | O | TRP | A | 126 | 43.852 | 43.445 | 145.266 | 1.00 | 0.00 | xxxx | 984 |
| ATOM | 985 | N | LYS | A | 127 | 45.926 | 43.882 | 146.013 | 1.00 | 0.00 | xxxx | 985 |
| ATOM | 986 | CA | LYS | A | 127 | 46.315 | 42.481 | 146.073 | 1.00 | 0.00 | xxxx | 986 |
| ATOM | 987 | CB | LYS | A | 127 | 47.795 | 42.368 | 146.436 | 1.00 | 0.00 | xxxx | 987 |
| ATOM | 988 | CG | LYS | A | 127 | 48.710 | 42.840 | 145.322 | 1.00 | 0.00 | xxxx | 988 |
| ATOM | 989 | CD | LYS | A | 127 | 50.162 | 42.861 | 145.766 | 1.00 | 0.00 | xxxx | 989 |
| ATOM | 990 | CE | LYS | A | 127 | 51.067 | 43.232 | 144.613 | 1.00 | 0.00 | xxxx | 990 |
| ATOM | 991 | NZ | LYS | A | 127 | 52.478 | 43.303 | 145.057 | 1.00 | 0.00 | xxxx | 991 |
| ATOM | 992 | C | LYS | A | 127 | 45.460 | 41.674 | 147.058 | 1.00 | 0.00 | xxxx | 992 |
| ATOM | 993 | O | LYS | A | 127 | 45.192 | 40.492 | 146.829 | 1.00 | 0.00 | xxxx | 993 |
| ATOM | 994 | N | ALA | A | 128 | 45.005 | 42.316 | 148.132 | 1.00 | 0.00 | xxxx | 994 |
| ATOM | 995 | CA | ALA | A | 128 | 44.264 | 41.614 | 149.176 | 1.00 | 0.00 | xxxx | 995 |
| ATOM | 996 | CB | ALA | A | 128 | 44.547 | 42.243 | 150.529 | 1.00 | 0.00 | xxxx | 996 |
| ATOM | 997 | C | ALA | A | 128 | 42.759 | 41.588 | 148.924 | 1.00 | 0.00 | xxxx | 997 |
| ATOM | 998 | O | ALA | A | 128 | 42.032 | 40.867 | 149.606 | 1.00 | 0.00 | xxxx | 998 |
| ATOM | 999 | N | HIS | A | 129 | 42.298 | 42.367 | 147.951 | 1.00 | 0.00 | xxxx | 999 |
| ATOM | 1000 | CA | HIS | A | 129 | 40.862 | 42.522 | 147.715 | 1.00 | 0.00 | xxxx | 1000 |
| ATOM | 1001 | CB | HIS | A | 129 | 40.403 | 43.904 | 148.198 | 1.00 | 0.00 | xxxx | 1001 |
| ATOM | 1002 | CG | HIS | A | 129 | 40.528 | 44.091 | 149.674 | 1.00 | 0.00 | xxxx | 1002 |
| ATOM | 1003 | ND1 | HIS | A | 129 | 41.703 | 44.486 | 150.278 | 1.00 | 0.00 | xxxx | 1003 |
| ATOM | 1004 | CE1 | HIS | A | 129 | 41.524 | 44.548 | 151.586 | 1.00 | 0.00 | xxxx | 1004 |
| ATOM | 1005 | NE2 | HIS | A | 129 | 40.280 | 44.196 | 151.854 | 1.00 | 0.00 | xxxx | 1005 |
| ATOM | 1006 | CD2 | HIS | A | 129 | 39.635 | 43.900 | 150.677 | 1.00 | 0.00 | xxxx | 1006 |
| ATOM | 1007 | C | HIS | A | 129 | 40.481 | 42.338 | 146.249 | 1.00 | 0.00 | xxxx | 1007 |
| ATOM | 1008 | O | HIS | A | 129 | 40.392 | 43.311 | 145.504 | 1.00 | 0.00 | xxxx | 1008 |
| ATOM | 1009 | N | PRO | A | 130 | 40.237 | 41.086 | 145.838 | 1.00 | 0.00 | xxxx | 1009 |
| ATOM | 1010 | CA | PRO | A | 130 | 39.859 | 40.757 | 144.457 | 1.00 | 0.00 | xxxx | 1010 |
| ATOM | 1011 | CB | PRO | A | 130 | 39.548 | 39.259 | 144.526 | 1.00 | 0.00 | xxxx | 1011 |
| ATOM | 1012 | CG | PRO | A | 130 | 40.266 | 38.768 | 145.740 | 1.00 | 0.00 | xxxx | 1012 |
| ATOM | 1013 | CD | PRO | A | 130 | 40.234 | 39.900 | 146.712 | 1.00 | 0.00 | xxxx | 1013 |
| ATOM | 1014 | C | PRO | A | 130 | 38.634 | 41.537 | 143.984 | 1.00 | 0.00 | xxxx | 1014 |
| ATOM | 1015 | O | PRO | A | 130 | 38.508 | 41.859 | 142.797 | 1.00 | 0.00 | xxxx | 1015 |
| ATOM | 1016 | N | GLU | A | 131 | 37.744 | 41.846 | 144.920 | 1.00 | 0.00 | xxxx | 1016 |
| ATOM | 1017 | CA | GLU | A | 131 | 36.507 | 42.546 | 144.601 | 1.00 | 0.00 | xxxx | 1017 |
| ATOM | 1018 | CB | GLU | A | 131 | 35.560 | 42.526 | 145.810 | 1.00 | 0.00 | xxxx | 1018 |
| ATOM | 1019 | CG | GLU | A | 131 | 35.970 | 43.431 | 146.972 | 1.00 | 0.00 | xxxx | 1019 |
| ATOM | 1020 | CD | GLU | A | 131 | 36.909 | 42.764 | 147.977 | 1.00 | 0.00 | xxxx | 1020 |
| ATOM | 1021 | OE1 | GLU | A | 131 | 37.546 | 41.737 | 147.647 | 1.00 | 0.00 | xxxx | 1021 |
| ATOM | 1022 | OE2 | GLU | A | 131 | 37.011 | 43.280 | 149.111 | 1.00 | 0.00 | xxxx | 1022 |
| ATOM | 1023 | C | GLU | A | 131 | 36.759 | 43.993 | 144.149 | 1.00 | 0.00 | xxxx | 1023 |
| ATOM | 1024 | O | GLU | A | 131 | 35.864 | 44.653 | 143.613 | 1.00 | 0.00 | xxxx | 1024 |
| ATOM | 1025 | N | ALA | A | 132 | 37.978 | 44.487 | 144.354 | 1.00 | 0.00 | xxxx | 1025 |
| ATOM | 1026 | CA | ALA | A | 132 | 38.301 | 45.870 | 143.999 | 1.00 | 0.00 | xxxx | 1026 |
| ATOM | 1027 | CB | ALA | A | 132 | 39.637 | 46.271 | 144.588 | 1.00 | 0.00 | xxxx | 1027 |
| ATOM | 1028 | C | ALA | A | 132 | 38.312 | 46.075 | 142.483 | 1.00 | 0.00 | xxxx | 1028 |
| ATOM | 1029 | O | ALA | A | 132 | 38.063 | 47.180 | 141.992 | 1.00 | 0.00 | xxxx | 1029 |
| ATOM | 1030 | N | ASP | A | 133 | 38.606 | 45.008 | 141.751 | 1.00 | 0.00 | xxxx | 1030 |
| ATOM | 1031 | CA | ASP | A | 133 | 38.589 | 45.039 | 140.296 | 1.00 | 0.00 | xxxx | 1031 |
| ATOM | 1032 | CB | ASP | A | 133 | 39.523 | 43.960 | 139.753 | 1.00 | 0.00 | xxxx | 1032 |
| ATOM | 1033 | CG | ASP | A | 133 | 39.634 | 43.977 | 138.253 | 1.00 | 0.00 | xxxx | 1033 |
| ATOM | 1034 | OD1 | ASP | A | 133 | 39.298 | 45.010 | 137.645 | 1.00 | 0.00 | xxxx | 1034 |
| ATOM | 1035 | OD2 | ASP | A | 133 | 40.082 | 42.949 | 137.695 | 1.00 | 0.00 | xxxx | 1035 |
| ATOM | 1036 | C | ASP | A | 133 | 37.141 | 44.847 | 139.833 | 1.00 | 0.00 | xxxx | 1036 |
| ATOM | 1037 | O | ASP | A | 133 | 36.727 | 43.749 | 139.436 | 1.00 | 0.00 | xxxx | 1037 |
| ATOM | 1038 | N | LYS | A | 134 | 36.381 | 45.936 | 139.891 | 1.00 | 0.00 | xxxx | 1038 |
| ATOM | 1039 | C | LYS | A | 134 | 34.486 | 45.450 | 138.329 | 1.00 | 0.00 | xxxx | 1039 |
| ATOM | 1040 | O | LYS | A | 134 | 33.414 | 44.858 | 138.173 | 1.00 | 0.00 | xxxx | 1040 |
| ATOM | 1041 | CA | LYS | A | 134 | 34.932 | 45.889 | 139.723 | 1.00 | 0.00 | xxxx | 1041 |
| ATOM | 1042 | CB | LYS | A | 134 | 34.341 | 47.257 | 140.067 | 1.00 | 0.00 | xxxx | 1042 |
| ATOM | 1043 | CG | LYS | A | 134 | 34.547 | 47.627 | 141.529 | 1.00 | 0.00 | xxxx | 1043 |
| ATOM | 1044 | CD | LYS | A | 134 | 34.923 | 49.089 | 141.712 | 1.00 | 0.00 | xxxx | 1044 |
| ATOM | 1045 | CE | LYS | A | 134 | 33.833 | 50.003 | 141.212 | 1.00 | 0.00 | xxxx | 1045 |
| ATOM | 1046 | NZ | LYS | A | 134 | 33.897 | 51.369 | 141.799 | 1.00 | 0.00 | xxxx | 1046 |
| ATOM | 1047 | N | ASN | A | 135 | 35.297 | 45.724 | 137.310 | 1.00 | 0.00 | xxxx | 1047 |
| ATOM | 1048 | CA | ASN | A | 135 | 34.922 | 45.292 | 135.961 | 1.00 | 0.00 | xxxx | 1048 |
| ATOM | 1049 | CB | ASN | A | 135 | 35.113 | 46.438 | 134.955 | 1.00 | 0.00 | xxxx | 1049 |
| ATOM | 1050 | CG | ASN | A | 135 | 36.566 | 46.696 | 134.618 | 1.00 | 0.00 | xxxx | 1050 |
| ATOM | 1051 | OD1 | ASN | A | 135 | 37.458 | 46.418 | 135.414 | 1.00 | 0.00 | xxxx | 1051 |
| ATOM | 1052 | ND2 | ASN | A | 135 | 36.813 | 47.232 | 133.425 | 1.00 | 0.00 | xxxx | 1052 |
| ATOM | 1053 | C | ASN | A | 135 | 35.693 | 44.043 | 135.519 | 1.00 | 0.00 | xxxx | 1053 |
| ATOM | 1054 | O | ASN | A | 135 | 35.605 | 43.631 | 134.364 | 1.00 | 0.00 | xxxx | 1054 |
| ATOM | 1055 | N | HIS | A | 136 | 36.439 | 43.448 | 136.450 | 1.00 | 0.00 | xxxx | 1055 |
| ATOM | 1056 | CA | HIS | A | 136 | 37.083 | 42.144 | 136.243 | 1.00 | 0.00 | xxxx | 1056 |
| ATOM | 1057 | CB | HIS | A | 136 | 36.021 | 41.045 | 136.121 | 1.00 | 0.00 | xxxx | 1057 |
| ATOM | 1058 | CG | HIS | A | 136 | 34.940 | 41.132 | 137.151 | 1.00 | 0.00 | xxxx | 1058 |
| ATOM | 1059 | ND1 | HIS | A | 136 | 35.154 | 40.840 | 138.482 | 1.00 | 0.00 | xxxx | 1059 |
| ATOM | 1060 | CE1 | HIS | A | 136 | 34.029 | 41.006 | 139.153 | 1.00 | 0.00 | xxxx | 1060 |
| ATOM | 1061 | NE2 | HIS | A | 136 | 33.090 | 41.389 | 138.305 | 1.00 | 0.00 | xxxx | 1061 |
| ATOM | 1062 | CD2 | HIS | A | 136 | 33.635 | 41.476 | 137.047 | 1.00 | 0.00 | xxxx | 1062 |
| ATOM | 1063 | C | HIS | A | 136 | 37.993 | 42.069 | 135.021 | 1.00 | 0.00 | xxxx | 1063 |
| ATOM | 1064 | O | HIS | A | 136 | 38.072 | 41.013 | 134.378 | 1.00 | 0.00 | xxxx | 1064 |
| ATOM | 1065 | N | ASP | A | 137 | 38.672 | 43.166 | 134.695 | 1.00 | 0.00 | xxxx | 1065 |
| ATOM | 1066 | CA | ASP | A | 137 | 39.557 | 43.181 | 133.529 | 1.00 | 0.00 | xxxx | 1066 |
| ATOM | 1067 | CB | ASP | A | 137 | 39.354 | 44.472 | 132.712 | 1.00 | 0.00 | xxxx | 1067 |
| ATOM | 1068 | CG | ASP | A | 137 | 39.756 | 45.740 | 133.464 | 1.00 | 0.00 | xxxx | 1068 |
| ATOM | 1069 | OD1 | ASP | A | 137 | 40.114 | 45.670 | 134.658 | 1.00 | 0.00 | xxxx | 1069 |
| ATOM | 1070 | OD2 | ASP | A | 137 | 39.711 | 46.818 | 132.834 | 1.00 | 0.00 | xxxx | 1070 |
| ATOM | 1071 | C | ASP | A | 137 | 41.033 | 43.012 | 133.892 | 1.00 | 0.00 | xxxx | 1071 |
| ATOM | 1072 | O | ASP | A | 137 | 41.893 | 43.016 | 133.011 | 1.00 | 0.00 | xxxx | 1072 |
| ATOM | 1073 | N | GLY | A | 138 | 41.320 | 42.860 | 135.183 | 1.00 | 0.00 | xxxx | 1073 |
| ATOM | 1074 | CA | GLY | A | 138 | 42.687 | 42.694 | 135.654 | 1.00 | 0.00 | xxxx | 1074 |
| ATOM | 1075 | C | GLY | A | 138 | 43.523 | 43.962 | 135.602 | 1.00 | 0.00 | xxxx | 1075 |
| ATOM | 1076 | O | GLY | A | 138 | 44.754 | 43.909 | 135.600 | 1.00 | 0.00 | xxxx | 1076 |
| ATOM | 1077 | N | VAL | A | 139 | 42.844 | 45.104 | 135.556 | 1.00 | 0.00 | xxxx | 1077 |
| ATOM | 1078 | CA | VAL | A | 139 | 43.486 | 46.412 | 135.479 | 1.00 | 0.00 | xxxx | 1078 |
| ATOM | 1079 | CB | VAL | A | 139 | 43.348 | 47.030 | 134.067 | 1.00 | 0.00 | xxxx | 1079 |
| ATOM | 1080 | CG1 | VAL | A | 139 | 44.016 | 48.399 | 134.003 | 1.00 | 0.00 | xxxx | 1080 |
| ATOM | 1081 | CG2 | VAL | A | 139 | 43.922 | 46.100 | 133.009 | 1.00 | 0.00 | xxxx | 1081 |
| ATOM | 1082 | C | VAL | A | 139 | 42.863 | 47.329 | 136.526 | 1.00 | 0.00 | xxxx | 1082 |
| ATOM | 1083 | O | VAL | A | 139 | 41.644 | 47.336 | 136.699 | 1.00 | 0.00 | xxxx | 1083 |
| ATOM | 1084 | N | MET | A | 140 | 43.691 | 48.096 | 137.228 | 1.00 | 0.00 | xxxx | 1084 |
| ATOM | 1085 | CA | MET | A | 140 | 43.187 | 49.070 | 138.191 | 1.00 | 0.00 | xxxx | 1085 |
| ATOM | 1086 | CB | MET | A | 140 | 44.219 | 49.322 | 139.293 | 1.00 | 0.00 | xxxx | 1086 |
| ATOM | 1087 | CG | MET | A | 140 | 43.815 | 50.406 | 140.290 | 1.00 | 0.00 | xxxx | 1087 |
| ATOM | 1088 | SD | MET | A | 140 | 45.208 | 50.999 | 141.305 | 1.00 | 0.00 | xxxx | 1088 |
| ATOM | 1089 | CE | MET | A | 140 | 45.540 | 49.546 | 142.295 | 1.00 | 0.00 | xxxx | 1089 |
| ATOM | 1090 | C | MET | A | 140 | 42.822 | 50.389 | 137.511 | 1.00 | 0.00 | xxxx | 1090 |
| ATOM | 1091 | O | MET | A | 140 | 43.702 | 51.147 | 137.098 | 1.00 | 0.00 | xxxx | 1091 |
| ATOM | 1092 | N | GLN | A | 141 | 41.525 | 50.655 | 137.382 | 1.00 | 0.00 | xxxx | 1092 |
| ATOM | 1093 | CA | GLN | A | 141 | 41.067 | 51.947 | 136.863 | 1.00 | 0.00 | xxxx | 1093 |
| ATOM | 1094 | CB | GLN | A | 141 | 39.638 | 51.857 | 136.329 | 1.00 | 0.00 | xxxx | 1094 |
| ATOM | 1095 | CG | GLN | A | 141 | 39.516 | 51.404 | 134.879 | 1.00 | 0.00 | xxxx | 1095 |
| ATOM | 1096 | CD | GLN | A | 141 | 39.714 | 49.912 | 134.703 | 1.00 | 0.00 | xxxx | 1096 |
| ATOM | 1097 | OE1 | GLN | A | 141 | 39.282 | 49.108 | 135.530 | 1.00 | 0.00 | xxxx | 1097 |
| ATOM | 1098 | NE2 | GLN | A | 141 | 40.352 | 49.531 | 133.602 | 1.00 | 0.00 | xxxx | 1098 |
| ATOM | 1099 | C | GLN | A | 141 | 41.121 | 52.998 | 137.958 | 1.00 | 0.00 | xxxx | 1099 |
| ATOM | 1100 | O | GLN | A | 141 | 40.443 | 52.855 | 138.974 | 1.00 | 0.00 | xxxx | 1100 |
| ATOM | 1101 | N | TYR | A | 142 | 41.888 | 54.065 | 137.752 | 1.00 | 0.00 | xxxx | 1101 |
| ATOM | 1102 | CA | TYR | A | 142 | 42.048 | 55.073 | 138.803 | 1.00 | 0.00 | xxxx | 1102 |
| ATOM | 1103 | CB | TYR | A | 142 | 43.434 | 54.948 | 139.458 | 1.00 | 0.00 | xxxx | 1103 |
| ATOM | 1104 | CG | TYR | A | 142 | 44.589 | 55.439 | 138.600 | 1.00 | 0.00 | xxxx | 1104 |
| ATOM | 1105 | CD1 | TYR | A | 142 | 45.024 | 56.767 | 138.666 | 1.00 | 0.00 | xxxx | 1105 |
| ATOM | 1106 | CE1 | TYR | A | 142 | 46.083 | 57.218 | 137.867 | 1.00 | 0.00 | xxxx | 1106 |
| ATOM | 1107 | CZ | TYR | A | 142 | 46.718 | 56.325 | 137.013 | 1.00 | 0.00 | xxxx | 1107 |
| ATOM | 1108 | OH | TYR | A | 142 | 47.762 | 56.736 | 136.211 | 1.00 | 0.00 | xxxx | 1108 |
| ATOM | 1109 | CE2 | TYR | A | 142 | 46.299 | 55.011 | 136.936 | 1.00 | 0.00 | xxxx | 1109 |
| ATOM | 1110 | CD2 | TYR | A | 142 | 45.249 | 54.574 | 137.728 | 1.00 | 0.00 | xxxx | 1110 |
| ATOM | 1111 | C | TYR | A | 142 | 41.857 | 56.500 | 138.303 | 1.00 | 0.00 | xxxx | 1111 |
| ATOM | 1112 | O | TYR | A | 142 | 41.923 | 56.769 | 137.102 | 1.00 | 0.00 | xxxx | 1112 |
| ATOM | 1113 | N | VAL | A | 143 | 41.623 | 57.415 | 139.242 | 1.00 | 0.00 | xxxx | 1113 |
| ATOM | 1114 | CA | VAL | A | 143 | 41.694 | 58.845 | 138.964 | 1.00 | 0.00 | xxxx | 1114 |
| ATOM | 1115 | CB | VAL | A | 143 | 40.337 | 59.563 | 139.132 | 1.00 | 0.00 | xxxx | 1115 |
| ATOM | 1116 | CG1 | VAL | A | 143 | 39.321 | 59.038 | 138.114 | 1.00 | 0.00 | xxxx | 1116 |
| ATOM | 1117 | CG2 | VAL | A | 143 | 39.815 | 59.418 | 140.565 | 1.00 | 0.00 | xxxx | 1117 |
| ATOM | 1118 | C | VAL | A | 143 | 42.743 | 59.449 | 139.882 | 1.00 | 0.00 | xxxx | 1118 |
| ATOM | 1119 | O | VAL | A | 143 | 43.052 | 58.898 | 140.939 | 1.00 | 0.00 | xxxx | 1119 |
| ATOM | 1120 | N | MET | A | 144 | 43.264 | 60.599 | 139.471 | 1.00 | 0.00 | xxxx | 1120 |
| ATOM | 1121 | CA | MET | A | 144 | 44.388 | 61.232 | 140.145 | 1.00 | 0.00 | xxxx | 1121 |
| ATOM | 1122 | CB | MET | A | 144 | 45.668 | 61.030 | 139.321 | 1.00 | 0.00 | xxxx | 1122 |
| ATOM | 1123 | CG | MET | A | 144 | 46.876 | 61.829 | 139.818 | 1.00 | 0.00 | xxxx | 1123 |
| ATOM | 1124 | SD | MET | A | 144 | 47.606 | 61.247 | 141.367 | 1.00 | 0.00 | xxxx | 1124 |
| ATOM | 1125 | CE | MET | A | 144 | 48.305 | 59.674 | 140.848 | 1.00 | 0.00 | xxxx | 1125 |
| ATOM | 1126 | C | MET | A | 144 | 44.118 | 62.717 | 140.359 | 1.00 | 0.00 | xxxx | 1126 |
| ATOM | 1127 | O | MET | A | 144 | 43.926 | 63.463 | 139.390 | 1.00 | 0.00 | xxxx | 1127 |
| ATOM | 1128 | N | LEU | A | 145 | 44.094 | 63.130 | 141.623 | 1.00 | 0.00 | xxxx | 1128 |
| ATOM | 1129 | CA | LEU | A | 145 | 43.927 | 64.537 | 141.981 | 1.00 | 0.00 | xxxx | 1129 |
| ATOM | 1130 | CB | LEU | A | 145 | 42.971 | 64.684 | 143.162 | 1.00 | 0.00 | xxxx | 1130 |
| ATOM | 1131 | CG | LEU | A | 145 | 41.522 | 64.394 | 142.788 | 1.00 | 0.00 | xxxx | 1131 |
| ATOM | 1132 | CD1 | LEU | A | 145 | 40.733 | 63.931 | 144.017 | 1.00 | 0.00 | xxxx | 1132 |
| ATOM | 1133 | CD2 | LEU | A | 145 | 40.908 | 65.648 | 142.174 | 1.00 | 0.00 | xxxx | 1133 |
| ATOM | 1134 | C | LEU | A | 145 | 45.291 | 65.124 | 142.311 | 1.00 | 0.00 | xxxx | 1134 |
| ATOM | 1135 | O | LEU | A | 145 | 45.892 | 64.771 | 143.333 | 1.00 | 0.00 | xxxx | 1135 |
| ATOM | 1136 | N | MET | A | 146 | 45.789 | 65.995 | 141.435 | 1.00 | 0.00 | xxxx | 1136 |
| ATOM | 1137 | CA | MET | A | 146 | 47.123 | 66.570 | 141.610 | 1.00 | 0.00 | xxxx | 1137 |
| ATOM | 1138 | CB | MET | A | 146 | 47.793 | 66.784 | 140.258 | 1.00 | 0.00 | xxxx | 1138 |
| ATOM | 1139 | CG | MET | A | 146 | 47.928 | 65.499 | 139.472 | 1.00 | 0.00 | xxxx | 1139 |
| ATOM | 1140 | SD | MET | A | 146 | 48.716 | 65.741 | 137.878 | 1.00 | 0.00 | xxxx | 1140 |
| ATOM | 1141 | CE | MET | A | 146 | 47.451 | 66.667 | 136.991 | 1.00 | 0.00 | xxxx | 1141 |
| ATOM | 1142 | C | MET | A | 146 | 47.070 | 67.882 | 142.377 | 1.00 | 0.00 | xxxx | 1142 |
| ATOM | 1143 | O | MET | A | 146 | 46.039 | 68.531 | 142.425 | 1.00 | 0.00 | xxxx | 1143 |
| ATOM | 1144 | N | GLY | A | 147 | 48.197 | 68.261 | 142.974 | 1.00 | 0.00 | xxxx | 1144 |
| ATOM | 1145 | CA | GLY | A | 147 | 48.327 | 69.550 | 143.634 | 1.00 | 0.00 | xxxx | 1145 |
| ATOM | 1146 | C | GLY | A | 147 | 48.417 | 70.715 | 142.657 | 1.00 | 0.00 | xxxx | 1146 |
| ATOM | 1147 | O | GLY | A | 147 | 47.771 | 70.705 | 141.619 | 1.00 | 0.00 | xxxx | 1147 |
| ATOM | 1148 | N | GLN | A | 148 | 49.205 | 71.732 | 142.986 | 1.00 | 0.00 | xxxx | 1148 |
| ATOM | 1149 | CA | GLN | A | 148 | 49.395 | 72.843 | 142.055 | 1.00 | 0.00 | xxxx | 1149 |
| ATOM | 1150 | CB | GLN | A | 148 | 49.902 | 74.083 | 142.796 | 1.00 | 0.00 | xxxx | 1150 |
| ATOM | 1151 | CG | GLN | A | 148 | 48.868 | 74.706 | 143.724 | 1.00 | 0.00 | xxxx | 1151 |
| ATOM | 1152 | CD | GLN | A | 148 | 49.487 | 75.729 | 144.659 | 1.00 | 0.00 | xxxx | 1152 |
| ATOM | 1153 | OE1 | GLN | A | 148 | 50.471 | 75.444 | 145.347 | 1.00 | 0.00 | xxxx | 1153 |
| ATOM | 1154 | NE2 | GLN | A | 148 | 48.921 | 76.939 | 144.678 | 1.00 | 0.00 | xxxx | 1154 |
| ATOM | 1155 | C | GLN | A | 148 | 50.374 | 72.477 | 140.953 | 1.00 | 0.00 | xxxx | 1155 |
| ATOM | 1156 | O | GLN | A | 148 | 51.385 | 71.828 | 141.210 | 1.00 | 0.00 | xxxx | 1156 |
| ATOM | 1157 | N | PRO | A | 149 | 50.104 | 72.936 | 139.721 | 1.00 | 0.00 | xxxx | 1157 |
| ATOM | 1158 | CA | PRO | A | 149 | 51.021 | 72.653 | 138.610 | 1.00 | 0.00 | xxxx | 1158 |
| ATOM | 1159 | CB | PRO | A | 149 | 50.440 | 73.464 | 137.451 | 1.00 | 0.00 | xxxx | 1159 |
| ATOM | 1160 | CG | PRO | A | 149 | 49.015 | 73.676 | 137.798 | 1.00 | 0.00 | xxxx | 1160 |
| ATOM | 1161 | CD | PRO | A | 149 | 48.921 | 73.696 | 139.294 | 1.00 | 0.00 | xxxx | 1161 |
| ATOM | 1162 | C | PRO | A | 149 | 52.447 | 73.111 | 138.915 | 1.00 | 0.00 | xxxx | 1162 |
| ATOM | 1163 | O | PRO | A | 149 | 52.656 | 74.234 | 139.367 | 1.00 | 0.00 | xxxx | 1163 |
| ATOM | 1164 | N | GLY | A | 150 | 53.414 | 72.235 | 138.687 | 1.00 | 0.00 | xxxx | 1164 |
| ATOM | 1165 | CA | GLY | A | 150 | 54.802 | 72.622 | 138.876 | 1.00 | 0.00 | xxxx | 1165 |
| ATOM | 1166 | C | GLY | A | 150 | 55.350 | 72.563 | 140.290 | 1.00 | 0.00 | xxxx | 1166 |
| ATOM | 1167 | O | GLY | A | 150 | 56.548 | 72.731 | 140.491 | 1.00 | 0.00 | xxxx | 1167 |
| ATOM | 1168 | N | HIS | A | 151 | 54.485 | 72.346 | 141.271 | 1.00 | 0.00 | xxxx | 1168 |
| ATOM | 1169 | CA | HIS | A | 151 | 54.938 | 72.119 | 142.640 | 1.00 | 0.00 | xxxx | 1169 |
| ATOM | 1170 | CB | HIS | A | 151 | 53.705 | 72.145 | 143.550 | 1.00 | 0.00 | xxxx | 1170 |
| ATOM | 1171 | CG | HIS | A | 151 | 53.984 | 72.117 | 145.018 | 1.00 | 0.00 | xxxx | 1171 |
| ATOM | 1172 | ND1 | HIS | A | 151 | 54.669 | 71.099 | 145.648 | 1.00 | 0.00 | xxxx | 1172 |
| ATOM | 1173 | CE1 | HIS | A | 151 | 54.689 | 71.325 | 146.947 | 1.00 | 0.00 | xxxx | 1173 |
| ATOM | 1174 | NE2 | HIS | A | 151 | 54.032 | 72.449 | 147.188 | 1.00 | 0.00 | xxxx | 1174 |
| ATOM | 1175 | CD2 | HIS | A | 151 | 53.580 | 72.963 | 146.001 | 1.00 | 0.00 | xxxx | 1175 |
| ATOM | 1176 | C | HIS | A | 151 | 55.671 | 70.774 | 142.681 | 1.00 | 0.00 | xxxx | 1176 |
| ATOM | 1177 | O | HIS | A | 151 | 55.152 | 69.796 | 142.145 | 1.00 | 0.00 | xxxx | 1177 |
| ATOM | 1178 | N | GLN | A | 152 | 56.857 | 70.706 | 143.293 | 1.00 | 0.00 | xxxx | 1178 |
| ATOM | 1179 | CA | GLN | A | 152 | 57.634 | 69.455 | 143.237 | 1.00 | 0.00 | xxxx | 1179 |
| ATOM | 1180 | CB | GLN | A | 152 | 59.023 | 69.636 | 143.877 | 1.00 | 0.00 | xxxx | 1180 |
| ATOM | 1181 | CG | GLN | A | 152 | 59.071 | 69.521 | 145.405 | 1.00 | 0.00 | xxxx | 1181 |
| ATOM | 1182 | CD | GLN | A | 152 | 58.525 | 70.739 | 146.127 | 1.00 | 0.00 | xxxx | 1182 |
| ATOM | 1183 | OE1 | GLN | A | 152 | 58.442 | 71.832 | 145.566 | 1.00 | 0.00 | xxxx | 1183 |
| ATOM | 1184 | NE2 | GLN | A | 152 | 58.159 | 70.552 | 147.391 | 1.00 | 0.00 | xxxx | 1184 |
| ATOM | 1185 | C | GLN | A | 152 | 56.899 | 68.263 | 143.880 | 1.00 | 0.00 | xxxx | 1185 |
| ATOM | 1186 | O | GLN | A | 152 | 57.065 | 67.118 | 143.452 | 1.00 | 0.00 | xxxx | 1186 |
| ATOM | 1187 | N | ASP | A | 153 | 56.078 | 68.516 | 144.891 | 1.00 | 0.00 | xxxx | 1187 |
| ATOM | 1188 | CA | ASP | A | 153 | 55.357 | 67.426 | 145.527 | 1.00 | 0.00 | xxxx | 1188 |
| ATOM | 1189 | CB | ASP | A | 153 | 54.754 | 67.867 | 146.859 | 1.00 | 0.00 | xxxx | 1189 |
| ATOM | 1190 | CG | ASP | A | 153 | 55.813 | 68.166 | 147.909 | 1.00 | 0.00 | xxxx | 1190 |
| ATOM | 1191 | OD1 | ASP | A | 153 | 56.988 | 67.774 | 147.732 | 1.00 | 0.00 | xxxx | 1191 |
| ATOM | 1192 | OD2 | ASP | A | 153 | 55.472 | 68.791 | 148.934 | 1.00 | 0.00 | xxxx | 1192 |
| ATOM | 1193 | C | ASP | A | 153 | 54.257 | 66.902 | 144.617 | 1.00 | 0.00 | xxxx | 1193 |
| ATOM | 1194 | O | ASP | A | 153 | 53.980 | 65.708 | 144.612 | 1.00 | 0.00 | xxxx | 1194 |
| ATOM | 1195 | N | ALA | A | 154 | 53.627 | 67.791 | 143.852 | 1.00 | 0.00 | xxxx | 1195 |
| ATOM | 1196 | CA | ALA | A | 154 | 52.591 | 67.356 | 142.925 | 1.00 | 0.00 | xxxx | 1196 |
| ATOM | 1197 | CB | ALA | A | 154 | 51.830 | 68.554 | 142.337 | 1.00 | 0.00 | xxxx | 1197 |
| ATOM | 1198 | C | ALA | A | 154 | 53.233 | 66.521 | 141.820 | 1.00 | 0.00 | xxxx | 1198 |
| ATOM | 1199 | O | ALA | A | 154 | 52.750 | 65.438 | 141.503 | 1.00 | 0.00 | xxxx | 1199 |
| ATOM | 1200 | N | ILE | A | 155 | 54.338 | 67.008 | 141.258 | 1.00 | 0.00 | xxxx | 1200 |
| ATOM | 1201 | CA | ILE | A | 155 | 55.012 | 66.292 | 140.177 | 1.00 | 0.00 | xxxx | 1201 |
| ATOM | 1202 | CB | ILE | A | 155 | 56.235 | 67.079 | 139.660 | 1.00 | 0.00 | xxxx | 1202 |
| ATOM | 1203 | CG1 | ILE | A | 155 | 55.791 | 68.403 | 139.039 | 1.00 | 0.00 | xxxx | 1203 |
| ATOM | 1204 | CD1 | ILE | A | 155 | 56.916 | 69.404 | 138.848 | 1.00 | 0.00 | xxxx | 1204 |
| ATOM | 1205 | CG2 | ILE | A | 155 | 57.012 | 66.273 | 138.618 | 1.00 | 0.00 | xxxx | 1205 |
| ATOM | 1206 | C | ILE | A | 155 | 55.431 | 64.905 | 140.652 | 1.00 | 0.00 | xxxx | 1206 |
| ATOM | 1207 | O | ILE | A | 155 | 55.131 | 63.892 | 140.014 | 1.00 | 0.00 | xxxx | 1207 |
| ATOM | 1208 | N | LEU | A | 156 | 56.125 | 64.864 | 141.785 | 1.00 | 0.00 | xxxx | 1208 |
| ATOM | 1209 | CA | LEU | A | 156 | 56.755 | 63.617 | 142.208 | 1.00 | 0.00 | xxxx | 1209 |
| ATOM | 1210 | CB | LEU | A | 156 | 57.933 | 63.924 | 143.141 | 1.00 | 0.00 | xxxx | 1210 |
| ATOM | 1211 | CG | LEU | A | 156 | 59.079 | 64.620 | 142.408 | 1.00 | 0.00 | xxxx | 1211 |
| ATOM | 1212 | CD1 | LEU | A | 156 | 60.088 | 65.153 | 143.415 | 1.00 | 0.00 | xxxx | 1212 |
| ATOM | 1213 | CD2 | LEU | A | 156 | 59.740 | 63.664 | 141.396 | 1.00 | 0.00 | xxxx | 1213 |
| ATOM | 1214 | C | LEU | A | 156 | 55.776 | 62.638 | 142.868 | 1.00 | 0.00 | xxxx | 1214 |
| ATOM | 1215 | O | LEU | A | 156 | 55.900 | 61.428 | 142.671 | 1.00 | 0.00 | xxxx | 1215 |
| ATOM | 1216 | N | ARG | A | 157 | 54.800 | 63.124 | 143.631 | 1.00 | 0.00 | xxxx | 1216 |
| ATOM | 1217 | CA | ARG | A | 157 | 53.834 | 62.195 | 144.216 | 1.00 | 0.00 | xxxx | 1217 |
| ATOM | 1218 | CB | ARG | A | 157 | 52.940 | 62.892 | 145.235 | 1.00 | 0.00 | xxxx | 1218 |
| ATOM | 1219 | CG | ARG | A | 157 | 53.664 | 63.219 | 146.531 | 1.00 | 0.00 | xxxx | 1219 |
| ATOM | 1220 | CD | ARG | A | 157 | 52.800 | 64.102 | 147.399 | 1.00 | 0.00 | xxxx | 1220 |
| ATOM | 1221 | NE | ARG | A | 157 | 53.528 | 64.587 | 148.570 | 1.00 | 0.00 | xxxx | 1221 |
| ATOM | 1222 | CZ | ARG | A | 157 | 53.102 | 65.579 | 149.343 | 1.00 | 0.00 | xxxx | 1222 |
| ATOM | 1223 | NH1 | ARG | A | 157 | 51.958 | 66.183 | 149.071 | 1.00 | 0.00 | xxxx | 1223 |
| ATOM | 1224 | NH2 | ARG | A | 157 | 53.823 | 65.977 | 150.380 | 1.00 | 0.00 | xxxx | 1224 |
| ATOM | 1225 | C | ARG | A | 157 | 52.995 | 61.548 | 143.128 | 1.00 | 0.00 | xxxx | 1225 |
| ATOM | 1226 | O | ARG | A | 157 | 52.681 | 60.361 | 143.205 | 1.00 | 0.00 | xxxx | 1226 |
| ATOM | 1227 | N | THR | A | 158 | 52.654 | 62.321 | 142.104 | 1.00 | 0.00 | xxxx | 1227 |
| ATOM | 1228 | CA | THR | A | 158 | 51.861 | 61.804 | 140.992 | 1.00 | 0.00 | xxxx | 1228 |
| ATOM | 1229 | CB | THR | A | 158 | 51.466 | 62.932 | 140.027 | 1.00 | 0.00 | xxxx | 1229 |
| ATOM | 1230 | OG1 | THR | A | 158 | 50.646 | 63.877 | 140.737 | 1.00 | 0.00 | xxxx | 1230 |
| ATOM | 1231 | CG2 | THR | A | 158 | 50.680 | 62.386 | 138.858 | 1.00 | 0.00 | xxxx | 1231 |
| ATOM | 1232 | C | THR | A | 158 | 52.626 | 60.721 | 140.239 | 1.00 | 0.00 | xxxx | 1232 |
| ATOM | 1233 | O | THR | A | 158 | 52.078 | 59.654 | 139.931 | 1.00 | 0.00 | xxxx | 1233 |
| ATOM | 1234 | N | GLN | A | 159 | 53.896 | 60.997 | 139.954 | 1.00 | 0.00 | xxxx | 1234 |
| ATOM | 1235 | CA | GLN | A | 159 | 54.742 | 60.073 | 139.220 | 1.00 | 0.00 | xxxx | 1235 |
| ATOM | 1236 | CB | GLN | A | 159 | 56.065 | 60.756 | 138.868 | 1.00 | 0.00 | xxxx | 1236 |
| ATOM | 1237 | CG | GLN | A | 159 | 57.079 | 59.851 | 138.200 | 1.00 | 0.00 | xxxx | 1237 |
| ATOM | 1238 | CD | GLN | A | 159 | 58.397 | 60.554 | 137.958 | 1.00 | 0.00 | xxxx | 1238 |
| ATOM | 1239 | OE1 | GLN | A | 159 | 58.507 | 61.772 | 138.132 | 1.00 | 0.00 | xxxx | 1239 |
| ATOM | 1240 | NE2 | GLN | A | 159 | 59.411 | 59.789 | 137.566 | 1.00 | 0.00 | xxxx | 1240 |
| ATOM | 1241 | C | GLN | A | 159 | 55.002 | 58.794 | 140.016 | 1.00 | 0.00 | xxxx | 1241 |
| ATOM | 1242 | O | GLN | A | 159 | 54.830 | 57.686 | 139.506 | 1.00 | 0.00 | xxxx | 1242 |
| ATOM | 1243 | N | TYR | A | 160 | 55.436 | 58.943 | 141.261 | 1.00 | 0.00 | xxxx | 1243 |
| ATOM | 1244 | CA | TYR | A | 160 | 55.930 | 57.774 | 141.985 | 1.00 | 0.00 | xxxx | 1244 |
| ATOM | 1245 | CB | TYR | A | 160 | 56.932 | 58.207 | 143.074 | 1.00 | 0.00 | xxxx | 1245 |
| ATOM | 1246 | CG | TYR | A | 160 | 58.324 | 58.434 | 142.513 | 1.00 | 0.00 | xxxx | 1246 |
| ATOM | 1247 | CD1 | TYR | A | 160 | 58.656 | 59.629 | 141.880 | 1.00 | 0.00 | xxxx | 1247 |
| ATOM | 1248 | CE1 | TYR | A | 160 | 59.922 | 59.829 | 141.345 | 1.00 | 0.00 | xxxx | 1248 |
| ATOM | 1249 | CZ | TYR | A | 160 | 60.872 | 58.831 | 141.445 | 1.00 | 0.00 | xxxx | 1249 |
| ATOM | 1250 | OH | TYR | A | 160 | 62.141 | 59.022 | 140.925 | 1.00 | 0.00 | xxxx | 1250 |
| ATOM | 1251 | CE2 | TYR | A | 160 | 60.565 | 57.634 | 142.059 | 1.00 | 0.00 | xxxx | 1251 |
| ATOM | 1252 | CD2 | TYR | A | 160 | 59.292 | 57.440 | 142.587 | 1.00 | 0.00 | xxxx | 1252 |
| ATOM | 1253 | C | TYR | A | 160 | 54.823 | 56.906 | 142.578 | 1.00 | 0.00 | xxxx | 1253 |
| ATOM | 1254 | O | TYR | A | 160 | 55.029 | 55.709 | 142.755 | 1.00 | 0.00 | xxxx | 1254 |
| ATOM | 1255 | N | SER | A | 161 | 53.653 | 57.473 | 142.867 | 1.00 | 0.00 | xxxx | 1255 |
| ATOM | 1256 | CA | SER | A | 161 | 52.555 | 56.642 | 143.365 | 1.00 | 0.00 | xxxx | 1256 |
| ATOM | 1257 | CB | SER | A | 161 | 51.334 | 57.481 | 143.755 | 1.00 | 0.00 | xxxx | 1257 |
| ATOM | 1258 | OG | SER | A | 161 | 50.896 | 58.302 | 142.672 | 1.00 | 0.00 | xxxx | 1258 |
| ATOM | 1259 | C | SER | A | 161 | 52.170 | 55.606 | 142.317 | 1.00 | 0.00 | xxxx | 1259 |
| ATOM | 1260 | O | SER | A | 161 | 52.085 | 54.412 | 142.615 | 1.00 | 0.00 | xxxx | 1260 |
| ATOM | 1261 | N | ILE | A | 162 | 51.975 | 56.056 | 141.081 | 1.00 | 0.00 | xxxx | 1261 |
| ATOM | 1262 | CA | ILE | A | 162 | 51.519 | 55.152 | 140.043 | 1.00 | 0.00 | xxxx | 1262 |
| ATOM | 1263 | CB | ILE | A | 162 | 50.948 | 55.919 | 138.835 | 1.00 | 0.00 | xxxx | 1263 |
| ATOM | 1264 | CG1 | ILE | A | 162 | 49.767 | 56.797 | 139.266 | 1.00 | 0.00 | xxxx | 1264 |
| ATOM | 1265 | CD1 | ILE | A | 162 | 48.691 | 56.079 | 140.082 | 1.00 | 0.00 | xxxx | 1265 |
| ATOM | 1266 | CG2 | ILE | A | 162 | 50.522 | 54.942 | 137.737 | 1.00 | 0.00 | xxxx | 1266 |
| ATOM | 1267 | C | ILE | A | 162 | 52.650 | 54.215 | 139.615 | 1.00 | 0.00 | xxxx | 1267 |
| ATOM | 1268 | O | ILE | A | 162 | 52.418 | 53.028 | 139.407 | 1.00 | 0.00 | xxxx | 1268 |
| ATOM | 1269 | N | GLN | A | 163 | 53.876 | 54.725 | 139.497 | 1.00 | 0.00 | xxxx | 1269 |
| ATOM | 1270 | CA | GLN | A | 163 | 54.979 | 53.845 | 139.124 | 1.00 | 0.00 | xxxx | 1270 |
| ATOM | 1271 | CB | GLN | A | 163 | 56.292 | 54.614 | 138.981 | 1.00 | 0.00 | xxxx | 1271 |
| ATOM | 1272 | CG | GLN | A | 163 | 57.425 | 53.743 | 138.413 | 1.00 | 0.00 | xxxx | 1272 |
| ATOM | 1273 | CD | GLN | A | 163 | 57.069 | 53.143 | 137.053 | 1.00 | 0.00 | xxxx | 1273 |
| ATOM | 1274 | OE1 | GLN | A | 163 | 56.595 | 53.845 | 136.160 | 1.00 | 0.00 | xxxx | 1274 |
| ATOM | 1275 | NE2 | GLN | A | 163 | 57.275 | 51.836 | 136.903 | 1.00 | 0.00 | xxxx | 1275 |
| ATOM | 1276 | C | GLN | A | 163 | 55.141 | 52.726 | 140.150 | 1.00 | 0.00 | xxxx | 1276 |
| ATOM | 1277 | O | GLN | A | 163 | 55.471 | 51.597 | 139.790 | 1.00 | 0.00 | xxxx | 1277 |
| ATOM | 1278 | N | THR | A | 164 | 54.865 | 53.035 | 141.418 | 1.00 | 0.00 | xxxx | 1278 |
| ATOM | 1279 | CA | THR | A | 164 | 55.003 | 52.045 | 142.476 | 1.00 | 0.00 | xxxx | 1279 |
| ATOM | 1280 | CB | THR | A | 164 | 54.952 | 52.716 | 143.864 | 1.00 | 0.00 | xxxx | 1280 |
| ATOM | 1281 | OG1 | THR | A | 164 | 56.123 | 53.530 | 144.020 | 1.00 | 0.00 | xxxx | 1281 |
| ATOM | 1282 | CG2 | THR | A | 164 | 54.924 | 51.673 | 144.980 | 1.00 | 0.00 | xxxx | 1282 |
| ATOM | 1283 | C | THR | A | 164 | 53.940 | 50.949 | 142.361 | 1.00 | 0.00 | xxxx | 1283 |
| ATOM | 1284 | O | THR | A | 164 | 54.239 | 49.769 | 142.563 | 1.00 | 0.00 | xxxx | 1284 |
| ATOM | 1285 | N | VAL | A | 165 | 52.712 | 51.338 | 142.017 | 1.00 | 0.00 | xxxx | 1285 |
| ATOM | 1286 | CA | VAL | A | 165 | 51.667 | 50.354 | 141.732 | 1.00 | 0.00 | xxxx | 1286 |
| ATOM | 1287 | CB | VAL | A | 165 | 50.323 | 51.038 | 141.438 | 1.00 | 0.00 | xxxx | 1287 |
| ATOM | 1288 | CG1 | VAL | A | 165 | 49.261 | 49.993 | 141.094 | 1.00 | 0.00 | xxxx | 1288 |
| ATOM | 1289 | CG2 | VAL | A | 165 | 49.889 | 51.838 | 142.653 | 1.00 | 0.00 | xxxx | 1289 |
| ATOM | 1290 | C | VAL | A | 165 | 52.081 | 49.447 | 140.565 | 1.00 | 0.00 | xxxx | 1290 |
| ATOM | 1291 | O | VAL | A | 165 | 51.934 | 48.224 | 140.647 | 1.00 | 0.00 | xxxx | 1291 |
| ATOM | 1292 | N | LYS | A | 166 | 52.596 | 50.028 | 139.482 | 1.00 | 0.00 | xxxx | 1292 |
| ATOM | 1293 | CA | LYS | A | 166 | 53.042 | 49.206 | 138.357 | 1.00 | 0.00 | xxxx | 1293 |
| ATOM | 1294 | CB | LYS | A | 166 | 53.510 | 50.065 | 137.180 | 1.00 | 0.00 | xxxx | 1294 |
| ATOM | 1295 | CG | LYS | A | 166 | 52.445 | 50.933 | 136.540 | 1.00 | 0.00 | xxxx | 1295 |
| ATOM | 1296 | CD | LYS | A | 166 | 53.072 | 51.745 | 135.414 | 1.00 | 0.00 | xxxx | 1296 |
| ATOM | 1297 | CE | LYS | A | 166 | 52.078 | 52.671 | 134.758 | 1.00 | 0.00 | xxxx | 1297 |
| ATOM | 1298 | NZ | LYS | A | 166 | 52.729 | 53.514 | 133.706 | 1.00 | 0.00 | xxxx | 1298 |
| ATOM | 1299 | C | LYS | A | 166 | 54.179 | 48.276 | 138.777 | 1.00 | 0.00 | xxxx | 1299 |
| ATOM | 1300 | O | LYS | A | 166 | 54.214 | 47.105 | 138.386 | 1.00 | 0.00 | xxxx | 1300 |
| ATOM | 1301 | N | ASP | A | 167 | 55.107 | 48.802 | 139.571 | 1.00 | 0.00 | xxxx | 1301 |
| ATOM | 1302 | CA | ASP | A | 167 | 56.255 | 48.020 | 140.029 | 1.00 | 0.00 | xxxx | 1302 |
| ATOM | 1303 | CB | ASP | A | 167 | 57.224 | 48.907 | 140.805 | 1.00 | 0.00 | xxxx | 1303 |
| ATOM | 1304 | CG | ASP | A | 167 | 57.973 | 49.879 | 139.907 | 1.00 | 0.00 | xxxx | 1304 |
| ATOM | 1305 | OD1 | ASP | A | 167 | 57.921 | 49.715 | 138.667 | 1.00 | 0.00 | xxxx | 1305 |
| ATOM | 1306 | OD2 | ASP | A | 167 | 58.618 | 50.801 | 140.447 | 1.00 | 0.00 | xxxx | 1306 |
| ATOM | 1307 | C | ASP | A | 167 | 55.820 | 46.832 | 140.892 | 1.00 | 0.00 | xxxx | 1307 |
| ATOM | 1308 | O | ASP | A | 167 | 56.531 | 45.832 | 140.998 | 1.00 | 0.00 | xxxx | 1308 |
| ATOM | 1309 | N | ALA | A | 168 | 54.642 | 46.951 | 141.502 | 1.00 | 0.00 | xxxx | 1309 |
| ATOM | 1310 | CA | ALA | A | 168 | 54.075 | 45.895 | 142.326 | 1.00 | 0.00 | xxxx | 1310 |
| ATOM | 1311 | CB | ALA | A | 168 | 53.060 | 46.481 | 143.302 | 1.00 | 0.00 | xxxx | 1311 |
| ATOM | 1312 | C | ALA | A | 168 | 53.428 | 44.798 | 141.477 | 1.00 | 0.00 | xxxx | 1312 |
| ATOM | 1313 | O | ALA | A | 168 | 52.883 | 43.830 | 142.008 | 1.00 | 0.00 | xxxx | 1313 |
| ATOM | 1314 | N | GLY | A | 169 | 53.476 | 44.965 | 140.157 | 1.00 | 0.00 | xxxx | 1314 |
| ATOM | 1315 | CA | GLY | A | 169 | 52.955 | 43.965 | 139.241 | 1.00 | 0.00 | xxxx | 1315 |
| ATOM | 1316 | C | GLY | A | 169 | 51.511 | 44.173 | 138.819 | 1.00 | 0.00 | xxxx | 1316 |
| ATOM | 1317 | O | GLY | A | 169 | 50.890 | 43.274 | 138.249 | 1.00 | 0.00 | xxxx | 1317 |
| ATOM | 1318 | N | ILE | A | 170 | 50.979 | 45.360 | 139.091 | 1.00 | 0.00 | xxxx | 1318 |
| ATOM | 1319 | CA | ILE | A | 170 | 49.571 | 45.657 | 138.823 | 1.00 | 0.00 | xxxx | 1319 |
| ATOM | 1320 | CB | ILE | A | 170 | 48.918 | 46.348 | 140.042 | 1.00 | 0.00 | xxxx | 1320 |
| ATOM | 1321 | CG1 | ILE | A | 170 | 48.984 | 45.434 | 141.273 | 1.00 | 0.00 | xxxx | 1321 |
| ATOM | 1322 | CD1 | ILE | A | 170 | 48.785 | 46.204 | 142.574 | 1.00 | 0.00 | xxxx | 1322 |
| ATOM | 1323 | CG2 | ILE | A | 170 | 47.468 | 46.788 | 139.749 | 1.00 | 0.00 | xxxx | 1323 |
| ATOM | 1324 | C | ILE | A | 170 | 49.440 | 46.523 | 137.575 | 1.00 | 0.00 | xxxx | 1324 |
| ATOM | 1325 | O | ILE | A | 170 | 50.110 | 47.543 | 137.457 | 1.00 | 0.00 | xxxx | 1325 |
| ATOM | 1326 | N | LYS | A | 171 | 48.578 | 46.111 | 136.647 | 1.00 | 0.00 | xxxx | 1326 |
| ATOM | 1327 | CA | LYS | A | 171 | 48.273 | 46.927 | 135.474 | 1.00 | 0.00 | xxxx | 1327 |
| ATOM | 1328 | CB | LYS | A | 171 | 47.663 | 46.079 | 134.357 | 1.00 | 0.00 | xxxx | 1328 |
| ATOM | 1329 | CG | LYS | A | 171 | 48.611 | 45.042 | 133.779 | 1.00 | 0.00 | xxxx | 1329 |
| ATOM | 1330 | CD | LYS | A | 171 | 47.900 | 44.207 | 132.722 | 1.00 | 0.00 | xxxx | 1330 |
| ATOM | 1331 | CE | LYS | A | 171 | 48.757 | 43.051 | 132.242 | 1.00 | 0.00 | xxxx | 1331 |
| ATOM | 1332 | NZ | LYS | A | 171 | 47.987 | 42.147 | 131.340 | 1.00 | 0.00 | xxxx | 1332 |
| ATOM | 1333 | C | LYS | A | 171 | 47.314 | 48.042 | 135.859 | 1.00 | 0.00 | xxxx | 1333 |
| ATOM | 1334 | O | LYS | A | 171 | 46.385 | 47.832 | 136.642 | 1.00 | 0.00 | xxxx | 1334 |
| ATOM | 1335 | N | VAL | A | 172 | 47.536 | 49.231 | 135.310 | 1.00 | 0.00 | xxxx | 1335 |
| ATOM | 1336 | CA | VAL | A | 172 | 46.716 | 50.378 | 135.681 | 1.00 | 0.00 | xxxx | 1336 |
| ATOM | 1337 | CB | VAL | A | 172 | 47.507 | 51.384 | 136.554 | 1.00 | 0.00 | xxxx | 1337 |
| ATOM | 1338 | CG1 | VAL | A | 172 | 48.094 | 50.686 | 137.780 | 1.00 | 0.00 | xxxx | 1338 |
| ATOM | 1339 | CG2 | VAL | A | 172 | 48.598 | 52.069 | 135.743 | 1.00 | 0.00 | xxxx | 1339 |
| ATOM | 1340 | C | VAL | A | 172 | 46.164 | 51.080 | 134.451 | 1.00 | 0.00 | xxxx | 1340 |
| ATOM | 1341 | O | VAL | A | 172 | 46.700 | 50.963 | 133.346 | 1.00 | 0.00 | xxxx | 1341 |
| ATOM | 1342 | N | GLN | A | 173 | 45.080 | 51.818 | 134.661 | 1.00 | 0.00 | xxxx | 1342 |
| ATOM | 1343 | CA | GLN | A | 173 | 44.502 | 52.657 | 133.631 | 1.00 | 0.00 | xxxx | 1343 |
| ATOM | 1344 | CB | GLN | A | 173 | 43.334 | 51.963 | 132.930 | 1.00 | 0.00 | xxxx | 1344 |
| ATOM | 1345 | CG | GLN | A | 173 | 42.782 | 52.813 | 131.799 | 1.00 | 0.00 | xxxx | 1345 |
| ATOM | 1346 | CD | GLN | A | 173 | 41.612 | 52.175 | 131.091 | 1.00 | 0.00 | xxxx | 1346 |
| ATOM | 1347 | OE1 | GLN | A | 173 | 40.758 | 51.538 | 131.713 | 1.00 | 0.00 | xxxx | 1347 |
| ATOM | 1348 | NE2 | GLN | A | 173 | 41.567 | 52.334 | 129.773 | 1.00 | 0.00 | xxxx | 1348 |
| ATOM | 1349 | C | GLN | A | 173 | 44.033 | 53.957 | 134.253 | 1.00 | 0.00 | xxxx | 1349 |
| ATOM | 1350 | O | GLN | A | 173 | 43.168 | 53.956 | 135.129 | 1.00 | 0.00 | xxxx | 1350 |
| ATOM | 1351 | N | GLU | A | 174 | 44.604 | 55.060 | 133.783 | 1.00 | 0.00 | xxxx | 1351 |
| ATOM | 1352 | CA | GLU | A | 174 | 44.240 | 56.389 | 134.240 | 1.00 | 0.00 | xxxx | 1352 |
| ATOM | 1353 | CB | GLU | A | 174 | 45.385 | 57.356 | 133.963 | 1.00 | 0.00 | xxxx | 1353 |
| ATOM | 1354 | CG | GLU | A | 174 | 45.220 | 58.707 | 134.592 | 1.00 | 0.00 | xxxx | 1354 |
| ATOM | 1355 | CD | GLU | A | 174 | 46.487 | 59.534 | 134.440 | 1.00 | 0.00 | xxxx | 1355 |
| ATOM | 1356 | OE1 | GLU | A | 174 | 46.579 | 60.296 | 133.463 | 1.00 | 0.00 | xxxx | 1356 |
| ATOM | 1357 | OE2 | GLU | A | 174 | 47.400 | 59.384 | 135.278 | 1.00 | 0.00 | xxxx | 1357 |
| ATOM | 1358 | C | GLU | A | 174 | 42.967 | 56.881 | 133.552 | 1.00 | 0.00 | xxxx | 1358 |
| ATOM | 1359 | O | GLU | A | 174 | 42.996 | 57.233 | 132.374 | 1.00 | 0.00 | xxxx | 1359 |
| ATOM | 1360 | N | LEU | A | 175 | 41.852 | 56.899 | 134.274 | 1.00 | 0.00 | xxxx | 1360 |
| ATOM | 1361 | CA | LEU | A | 175 | 40.604 | 57.387 | 133.681 | 1.00 | 0.00 | xxxx | 1361 |
| ATOM | 1362 | CB | LEU | A | 175 | 39.398 | 56.922 | 134.498 | 1.00 | 0.00 | xxxx | 1362 |
| ATOM | 1363 | CG | LEU | A | 175 | 39.121 | 55.423 | 134.515 | 1.00 | 0.00 | xxxx | 1363 |
| ATOM | 1364 | CD1 | LEU | A | 175 | 37.913 | 55.154 | 135.402 | 1.00 | 0.00 | xxxx | 1364 |
| ATOM | 1365 | CD2 | LEU | A | 175 | 38.873 | 54.925 | 133.104 | 1.00 | 0.00 | xxxx | 1365 |
| ATOM | 1366 | C | LEU | A | 175 | 40.586 | 58.906 | 133.569 | 1.00 | 0.00 | xxxx | 1366 |
| ATOM | 1367 | O | LEU | A | 175 | 40.021 | 59.462 | 132.624 | 1.00 | 0.00 | xxxx | 1367 |
| ATOM | 1368 | N | ALA | A | 176 | 41.186 | 59.574 | 134.550 | 1.00 | 0.00 | xxxx | 1368 |
| ATOM | 1369 | CA | ALA | A | 176 | 41.232 | 61.036 | 134.576 | 1.00 | 0.00 | xxxx | 1369 |
| ATOM | 1370 | CB | ALA | A | 176 | 39.880 | 61.605 | 134.965 | 1.00 | 0.00 | xxxx | 1370 |
| ATOM | 1371 | C | ALA | A | 176 | 42.285 | 61.520 | 135.544 | 1.00 | 0.00 | xxxx | 1371 |
| ATOM | 1372 | O | ALA | A | 176 | 42.571 | 60.864 | 136.535 | 1.00 | 0.00 | xxxx | 1372 |
| ATOM | 1373 | N | LYS | A | 177 | 42.860 | 62.676 | 135.254 | 1.00 | 0.00 | xxxx | 1373 |
| ATOM | 1374 | CA | LYS | A | 177 | 43.719 | 63.346 | 136.213 | 1.00 | 0.00 | xxxx | 1374 |
| ATOM | 1375 | CB | LYS | A | 177 | 45.181 | 62.923 | 136.061 | 1.00 | 0.00 | xxxx | 1375 |
| ATOM | 1376 | CG | LYS | A | 177 | 45.871 | 63.428 | 134.814 | 1.00 | 0.00 | xxxx | 1376 |
| ATOM | 1377 | CD | LYS | A | 177 | 47.373 | 63.195 | 134.903 | 1.00 | 0.00 | xxxx | 1377 |
| ATOM | 1378 | CE | LYS | A | 177 | 48.041 | 63.494 | 133.577 | 1.00 | 0.00 | xxxx | 1378 |
| ATOM | 1379 | NZ | LYS | A | 177 | 49.468 | 63.096 | 133.605 | 1.00 | 0.00 | xxxx | 1379 |
| ATOM | 1380 | C | LYS | A | 177 | 43.561 | 64.833 | 136.011 | 1.00 | 0.00 | xxxx | 1380 |
| ATOM | 1381 | O | LYS | A | 177 | 43.310 | 65.291 | 134.901 | 1.00 | 0.00 | xxxx | 1381 |
| ATOM | 1382 | N | ASP | A | 178 | 43.663 | 65.595 | 137.089 | 1.00 | 0.00 | xxxx | 1382 |
| ATOM | 1383 | CA | ASP | A | 178 | 43.572 | 67.044 | 136.961 | 1.00 | 0.00 | xxxx | 1383 |
| ATOM | 1384 | CB | ASP | A | 178 | 42.114 | 67.492 | 136.772 | 1.00 | 0.00 | xxxx | 1384 |
| ATOM | 1385 | CG | ASP | A | 178 | 41.987 | 68.845 | 136.073 | 1.00 | 0.00 | xxxx | 1385 |
| ATOM | 1386 | OD1 | ASP | A | 178 | 43.015 | 69.536 | 135.869 | 1.00 | 0.00 | xxxx | 1386 |
| ATOM | 1387 | OD2 | ASP | A | 178 | 40.839 | 69.229 | 135.735 | 1.00 | 0.00 | xxxx | 1387 |
| ATOM | 1388 | C | ASP | A | 178 | 44.166 | 67.669 | 138.200 | 1.00 | 0.00 | xxxx | 1388 |
| ATOM | 1389 | O | ASP | A | 178 | 44.343 | 67.010 | 139.226 | 1.00 | 0.00 | xxxx | 1389 |
| ATOM | 1390 | N | TYR | A | 179 | 44.462 | 68.956 | 138.084 | 1.00 | 0.00 | xxxx | 1390 |
| ATOM | 1391 | CA | TYR | A | 179 | 44.969 | 69.751 | 139.184 | 1.00 | 0.00 | xxxx | 1391 |
| ATOM | 1392 | CB | TYR | A | 179 | 45.795 | 70.923 | 138.647 | 1.00 | 0.00 | xxxx | 1392 |
| ATOM | 1393 | CG | TYR | A | 179 | 47.028 | 70.506 | 137.884 | 1.00 | 0.00 | xxxx | 1393 |
| ATOM | 1394 | CD1 | TYR | A | 179 | 48.176 | 70.081 | 138.553 | 1.00 | 0.00 | xxxx | 1394 |
| ATOM | 1395 | CE1 | TYR | A | 179 | 49.308 | 69.699 | 137.851 | 1.00 | 0.00 | xxxx | 1395 |
| ATOM | 1396 | CZ | TYR | A | 179 | 49.294 | 69.751 | 136.473 | 1.00 | 0.00 | xxxx | 1396 |
| ATOM | 1397 | OH | TYR | A | 179 | 50.413 | 69.378 | 135.756 | 1.00 | 0.00 | xxxx | 1397 |
| ATOM | 1398 | CE2 | TYR | A | 179 | 48.175 | 70.174 | 135.792 | 1.00 | 0.00 | xxxx | 1398 |
| ATOM | 1399 | CD2 | TYR | A | 179 | 47.049 | 70.545 | 136.495 | 1.00 | 0.00 | xxxx | 1399 |
| ATOM | 1400 | C | TYR | A | 179 | 43.834 | 70.278 | 140.038 | 1.00 | 0.00 | xxxx | 1400 |
| ATOM | 1401 | O | TYR | A | 179 | 42.921 | 70.928 | 139.524 | 1.00 | 0.00 | xxxx | 1401 |
| ATOM | 1402 | N | ALA | A | 180 | 43.899 | 70.014 | 141.344 | 1.00 | 0.00 | xxxx | 1402 |
| ATOM | 1403 | CA | ALA | A | 180 | 42.958 | 70.631 | 142.268 | 1.00 | 0.00 | xxxx | 1403 |
| ATOM | 1404 | CB | ALA | A | 180 | 42.086 | 69.574 | 142.946 | 1.00 | 0.00 | xxxx | 1404 |
| ATOM | 1405 | C | ALA | A | 180 | 43.674 | 71.497 | 143.305 | 1.00 | 0.00 | xxxx | 1405 |
| ATOM | 1406 | O | ALA | A | 180 | 43.052 | 71.949 | 144.258 | 1.00 | 0.00 | xxxx | 1406 |
| ATOM | 1407 | N | ASN | A | 181 | 44.977 | 71.723 | 143.111 | 1.00 | 0.00 | xxxx | 1407 |
| ATOM | 1408 | CA | ASN | A | 181 | 45.678 | 72.840 | 143.766 | 1.00 | 0.00 | xxxx | 1408 |
| ATOM | 1409 | CB | ASN | A | 181 | 45.029 | 74.165 | 143.361 | 1.00 | 0.00 | xxxx | 1409 |
| ATOM | 1410 | CG | ASN | A | 181 | 44.936 | 74.317 | 141.862 | 1.00 | 0.00 | xxxx | 1410 |
| ATOM | 1411 | OD1 | ASN | A | 181 | 45.904 | 74.057 | 141.140 | 1.00 | 0.00 | xxxx | 1411 |
| ATOM | 1412 | ND2 | ASN | A | 181 | 43.759 | 74.698 | 141.378 | 1.00 | 0.00 | xxxx | 1412 |
| ATOM | 1413 | C | ASN | A | 181 | 45.728 | 72.755 | 145.284 | 1.00 | 0.00 | xxxx | 1413 |
| ATOM | 1414 | O | ASN | A | 181 | 45.785 | 73.792 | 145.959 | 1.00 | 0.00 | xxxx | 1414 |
| ATOM | 1415 | N | TRP | A | 182 | 45.695 | 71.520 | 145.796 | 1.00 | 0.00 | xxxx | 1415 |
| ATOM | 1416 | CA | TRP | A | 182 | 45.769 | 71.207 | 147.235 | 1.00 | 0.00 | xxxx | 1416 |
| ATOM | 1417 | CB | TRP | A | 182 | 46.918 | 71.965 | 147.932 | 1.00 | 0.00 | xxxx | 1417 |
| ATOM | 1418 | CG | TRP | A | 182 | 48.266 | 71.865 | 147.262 | 1.00 | 0.00 | xxxx | 1418 |
| ATOM | 1419 | CD1 | TRP | A | 182 | 49.010 | 72.896 | 146.766 | 1.00 | 0.00 | xxxx | 1419 |
| ATOM | 1420 | NE1 | TRP | A | 182 | 50.186 | 72.424 | 146.240 | 1.00 | 0.00 | xxxx | 1420 |
| ATOM | 1421 | CE2 | TRP | A | 182 | 50.237 | 71.065 | 146.408 | 1.00 | 0.00 | xxxx | 1421 |
| ATOM | 1422 | CD2 | TRP | A | 182 | 49.041 | 70.676 | 147.049 | 1.00 | 0.00 | xxxx | 1422 |
| ATOM | 1423 | CE3 | TRP | A | 182 | 48.840 | 69.321 | 147.344 | 1.00 | 0.00 | xxxx | 1423 |
| ATOM | 1424 | CZ3 | TRP | A | 182 | 49.825 | 68.411 | 146.987 | 1.00 | 0.00 | xxxx | 1424 |
| ATOM | 1425 | CH2 | TRP | A | 182 | 51.010 | 68.831 | 146.342 | 1.00 | 0.00 | xxxx | 1425 |
| ATOM | 1426 | CZ2 | TRP | A | 182 | 51.234 | 70.149 | 146.052 | 1.00 | 0.00 | xxxx | 1426 |
| ATOM | 1427 | C | TRP | A | 182 | 44.475 | 71.519 | 147.973 | 1.00 | 0.00 | xxxx | 1427 |
| ATOM | 1428 | O | TRP | A | 182 | 44.418 | 71.383 | 149.201 | 1.00 | 0.00 | xxxx | 1428 |
| ATOM | 1429 | N | ASP | A | 183 | 43.448 | 71.898 | 147.218 | 1.00 | 0.00 | xxxx | 1429 |
| ATOM | 1430 | CA | ASP | A | 183 | 42.214 | 72.426 | 147.783 | 1.00 | 0.00 | xxxx | 1430 |
| ATOM | 1431 | CB | ASP | A | 183 | 41.686 | 73.566 | 146.907 | 1.00 | 0.00 | xxxx | 1431 |
| ATOM | 1432 | CG | ASP | A | 183 | 40.491 | 74.259 | 147.514 | 1.00 | 0.00 | xxxx | 1432 |
| ATOM | 1433 | OD1 | ASP | A | 183 | 40.565 | 74.648 | 148.697 | 1.00 | 0.00 | xxxx | 1433 |
| ATOM | 1434 | OD2 | ASP | A | 183 | 39.463 | 74.411 | 146.815 | 1.00 | 0.00 | xxxx | 1434 |
| ATOM | 1435 | C | ASP | A | 183 | 41.146 | 71.348 | 147.935 | 1.00 | 0.00 | xxxx | 1435 |
| ATOM | 1436 | O | ASP | A | 183 | 40.932 | 70.529 | 147.039 | 1.00 | 0.00 | xxxx | 1436 |
| ATOM | 1437 | N | ARG | A | 184 | 40.477 | 71.355 | 149.081 | 1.00 | 0.00 | xxxx | 1437 |
| ATOM | 1438 | C | ARG | A | 184 | 38.200 | 70.639 | 148.421 | 1.00 | 0.00 | xxxx | 1438 |
| ATOM | 1439 | O | ARG | A | 184 | 37.716 | 69.714 | 147.782 | 1.00 | 0.00 | xxxx | 1439 |
| ATOM | 1440 | CA | ARG | A | 184 | 39.402 | 70.411 | 149.347 | 1.00 | 0.00 | xxxx | 1440 |
| ATOM | 1441 | CB | ARG | A | 184 | 38.978 | 70.522 | 150.818 | 1.00 | 0.00 | xxxx | 1441 |
| ATOM | 1442 | CG | ARG | A | 184 | 37.971 | 69.475 | 151.277 | 1.00 | 0.00 | xxxx | 1442 |
| ATOM | 1443 | CD | ARG | A | 184 | 37.897 | 69.420 | 152.808 | 1.00 | 0.00 | xxxx | 1443 |
| ATOM | 1444 | NE | ARG | A | 184 | 36.959 | 68.400 | 153.274 | 1.00 | 0.00 | xxxx | 1444 |
| ATOM | 1445 | CZ | ARG | A | 184 | 36.970 | 67.868 | 154.492 | 1.00 | 0.00 | xxxx | 1445 |
| ATOM | 1446 | NH1 | ARG | A | 184 | 37.877 | 68.251 | 155.382 | 1.00 | 0.00 | xxxx | 1446 |
| ATOM | 1447 | NH2 | ARG | A | 184 | 36.070 | 66.948 | 154.820 | 1.00 | 0.00 | xxxx | 1447 |
| ATOM | 1448 | N | VAL | A | 185 | 37.723 | 71.878 | 148.352 | 1.00 | 0.00 | xxxx | 1448 |
| ATOM | 1449 | CA | VAL | A | 185 | 36.511 | 72.147 | 147.575 | 1.00 | 0.00 | xxxx | 1449 |
| ATOM | 1450 | CB | VAL | A | 185 | 35.988 | 73.561 | 147.828 | 1.00 | 0.00 | xxxx | 1450 |
| ATOM | 1451 | CG1 | VAL | A | 185 | 34.800 | 73.869 | 146.907 | 1.00 | 0.00 | xxxx | 1451 |
| ATOM | 1452 | CG2 | VAL | A | 185 | 35.568 | 73.687 | 149.283 | 1.00 | 0.00 | xxxx | 1452 |
| ATOM | 1453 | C | VAL | A | 185 | 36.750 | 71.902 | 146.088 | 1.00 | 0.00 | xxxx | 1453 |
| ATOM | 1454 | O | VAL | A | 185 | 35.893 | 71.334 | 145.400 | 1.00 | 0.00 | xxxx | 1454 |
| ATOM | 1455 | N | THR | A | 186 | 37.914 | 72.300 | 145.591 | 1.00 | 0.00 | xxxx | 1455 |
| ATOM | 1456 | CA | THR | A | 186 | 38.221 | 72.061 | 144.181 | 1.00 | 0.00 | xxxx | 1456 |
| ATOM | 1457 | CB | THR | A | 186 | 39.561 | 72.715 | 143.790 | 1.00 | 0.00 | xxxx | 1457 |
| ATOM | 1458 | OG1 | THR | A | 186 | 39.532 | 74.100 | 144.155 | 1.00 | 0.00 | xxxx | 1458 |
| ATOM | 1459 | CG2 | THR | A | 186 | 39.794 | 72.603 | 142.287 | 1.00 | 0.00 | xxxx | 1459 |
| ATOM | 1460 | C | THR | A | 186 | 38.258 | 70.567 | 143.864 | 1.00 | 0.00 | xxxx | 1460 |
| ATOM | 1461 | O | THR | A | 186 | 37.718 | 70.117 | 142.852 | 1.00 | 0.00 | xxxx | 1461 |
| ATOM | 1462 | N | ALA | A | 187 | 38.887 | 69.790 | 144.739 | 1.00 | 0.00 | xxxx | 1462 |
| ATOM | 1463 | CA | ALA | A | 187 | 38.937 | 68.349 | 144.555 | 1.00 | 0.00 | xxxx | 1463 |
| ATOM | 1464 | CB | ALA | A | 187 | 39.807 | 67.725 | 145.626 | 1.00 | 0.00 | xxxx | 1464 |
| ATOM | 1465 | C | ALA | A | 187 | 37.535 | 67.743 | 144.583 | 1.00 | 0.00 | xxxx | 1465 |
| ATOM | 1466 | O | ALA | A | 187 | 37.211 | 66.866 | 143.789 | 1.00 | 0.00 | xxxx | 1466 |
| ATOM | 1467 | N | HIS | A | 188 | 36.712 | 68.216 | 145.511 | 1.00 | 0.00 | xxxx | 1467 |
| ATOM | 1468 | CA | HIS | A | 188 | 35.322 | 67.782 | 145.572 | 1.00 | 0.00 | xxxx | 1468 |
| ATOM | 1469 | CB | HIS | A | 188 | 34.566 | 68.533 | 146.659 | 1.00 | 0.00 | xxxx | 1469 |
| ATOM | 1470 | CG | HIS | A | 188 | 33.084 | 68.377 | 146.558 | 1.00 | 0.00 | xxxx | 1470 |
| ATOM | 1471 | ND1 | HIS | A | 188 | 32.305 | 69.204 | 145.780 | 1.00 | 0.00 | xxxx | 1471 |
| ATOM | 1472 | CE1 | HIS | A | 188 | 31.041 | 68.820 | 145.871 | 1.00 | 0.00 | xxxx | 1472 |
| ATOM | 1473 | NE2 | HIS | A | 188 | 30.981 | 67.775 | 146.677 | 1.00 | 0.00 | xxxx | 1473 |
| ATOM | 1474 | CD2 | HIS | A | 188 | 32.247 | 67.473 | 147.117 | 1.00 | 0.00 | xxxx | 1474 |
| ATOM | 1475 | C | HIS | A | 188 | 34.633 | 67.985 | 144.232 | 1.00 | 0.00 | xxxx | 1475 |
| ATOM | 1476 | O | HIS | A | 188 | 33.967 | 67.082 | 143.731 | 1.00 | 0.00 | xxxx | 1476 |
| ATOM | 1477 | N | ASP | A | 189 | 34.806 | 69.170 | 143.657 | 1.00 | 0.00 | xxxx | 1477 |
| ATOM | 1478 | CA | ASP | A | 189 | 34.119 | 69.515 | 142.419 | 1.00 | 0.00 | xxxx | 1478 |
| ATOM | 1479 | CB | ASP | A | 189 | 34.260 | 71.012 | 142.121 | 1.00 | 0.00 | xxxx | 1479 |
| ATOM | 1480 | CG | ASP | A | 189 | 33.530 | 71.898 | 143.134 | 1.00 | 0.00 | xxxx | 1480 |
| ATOM | 1481 | OD1 | ASP | A | 189 | 32.775 | 71.381 | 143.997 | 1.00 | 0.00 | xxxx | 1481 |
| ATOM | 1482 | OD2 | ASP | A | 189 | 33.715 | 73.134 | 143.065 | 1.00 | 0.00 | xxxx | 1482 |
| ATOM | 1483 | C | ASP | A | 189 | 34.648 | 68.674 | 141.252 | 1.00 | 0.00 | xxxx | 1483 |
| ATOM | 1484 | O | ASP | A | 189 | 33.864 | 68.214 | 140.414 | 1.00 | 0.00 | xxxx | 1484 |
| ATOM | 1485 | N | LYS | A | 190 | 35.966 | 68.462 | 141.195 | 1.00 | 0.00 | xxxx | 1485 |
| ATOM | 1486 | CA | LYS | A | 190 | 36.543 | 67.604 | 140.153 | 1.00 | 0.00 | xxxx | 1486 |
| ATOM | 1487 | CB | LYS | A | 190 | 38.076 | 67.575 | 140.244 | 1.00 | 0.00 | xxxx | 1487 |
| ATOM | 1488 | CG | LYS | A | 190 | 38.773 | 68.916 | 140.085 | 1.00 | 0.00 | xxxx | 1488 |
| ATOM | 1489 | CD | LYS | A | 190 | 38.692 | 69.446 | 138.665 | 1.00 | 0.00 | xxxx | 1489 |
| ATOM | 1490 | CE | LYS | A | 190 | 39.552 | 70.698 | 138.487 | 1.00 | 0.00 | xxxx | 1490 |
| ATOM | 1491 | NZ | LYS | A | 190 | 39.457 | 71.199 | 137.081 | 1.00 | 0.00 | xxxx | 1491 |
| ATOM | 1492 | C | LYS | A | 190 | 36.000 | 66.178 | 140.260 | 1.00 | 0.00 | xxxx | 1492 |
| ATOM | 1493 | O | LYS | A | 190 | 35.587 | 65.570 | 139.263 | 1.00 | 0.00 | xxxx | 1493 |
| ATOM | 1494 | N | MET | A | 191 | 35.997 | 65.645 | 141.475 | 1.00 | 0.00 | xxxx | 1494 |
| ATOM | 1495 | CA | MET | A | 191 | 35.572 | 64.268 | 141.705 | 1.00 | 0.00 | xxxx | 1495 |
| ATOM | 1496 | CB | MET | A | 191 | 35.859 | 63.859 | 143.152 | 1.00 | 0.00 | xxxx | 1496 |
| ATOM | 1497 | CG | MET | A | 191 | 35.582 | 62.396 | 143.438 | 1.00 | 0.00 | xxxx | 1497 |
| ATOM | 1498 | SD | MET | A | 191 | 36.879 | 61.354 | 142.734 | 1.00 | 0.00 | xxxx | 1498 |
| ATOM | 1499 | CE | MET | A | 191 | 36.062 | 59.764 | 142.708 | 1.00 | 0.00 | xxxx | 1499 |
| ATOM | 1500 | C | MET | A | 191 | 34.089 | 64.078 | 141.388 | 1.00 | 0.00 | xxxx | 1500 |
| ATOM | 1501 | O | MET | A | 191 | 33.701 | 63.057 | 140.822 | 1.00 | 0.00 | xxxx | 1501 |
| ATOM | 1502 | N | ALA | A | 192 | 33.261 | 65.061 | 141.743 | 1.00 | 0.00 | xxxx | 1502 |
| ATOM | 1503 | CA | ALA | A | 192 | 31.831 | 64.962 | 141.469 | 1.00 | 0.00 | xxxx | 1503 |
| ATOM | 1504 | CB | ALA | A | 192 | 31.089 | 66.163 | 142.050 | 1.00 | 0.00 | xxxx | 1504 |
| ATOM | 1505 | C | ALA | A | 192 | 31.577 | 64.848 | 139.966 | 1.00 | 0.00 | xxxx | 1505 |
| ATOM | 1506 | O | ALA | A | 192 | 30.724 | 64.067 | 139.528 | 1.00 | 0.00 | xxxx | 1506 |
| ATOM | 1507 | N | ALA | A | 193 | 32.317 | 65.628 | 139.182 | 1.00 | 0.00 | xxxx | 1507 |
| ATOM | 1508 | CA | ALA | A | 193 | 32.166 | 65.611 | 137.730 | 1.00 | 0.00 | xxxx | 1508 |
| ATOM | 1509 | CB | ALA | A | 193 | 32.935 | 66.779 | 137.089 | 1.00 | 0.00 | xxxx | 1509 |
| ATOM | 1510 | C | ALA | A | 193 | 32.636 | 64.273 | 137.159 | 1.00 | 0.00 | xxxx | 1510 |
| ATOM | 1511 | O | ALA | A | 193 | 31.990 | 63.693 | 136.281 | 1.00 | 0.00 | xxxx | 1511 |
| ATOM | 1512 | N | TRP | A | 194 | 33.769 | 63.781 | 137.647 | 1.00 | 0.00 | xxxx | 1512 |
| ATOM | 1513 | CA | TRP | A | 194 | 34.223 | 62.454 | 137.258 | 1.00 | 0.00 | xxxx | 1513 |
| ATOM | 1514 | CB | TRP | A | 194 | 35.542 | 62.121 | 137.944 | 1.00 | 0.00 | xxxx | 1514 |
| ATOM | 1515 | CG | TRP | A | 194 | 36.704 | 62.930 | 137.447 | 1.00 | 0.00 | xxxx | 1515 |
| ATOM | 1516 | CD1 | TRP | A | 194 | 36.801 | 63.607 | 136.258 | 1.00 | 0.00 | xxxx | 1516 |
| ATOM | 1517 | NE1 | TRP | A | 194 | 38.028 | 64.222 | 136.163 | 1.00 | 0.00 | xxxx | 1517 |
| ATOM | 1518 | CE2 | TRP | A | 194 | 38.742 | 63.957 | 137.306 | 1.00 | 0.00 | xxxx | 1518 |
| ATOM | 1519 | CD2 | TRP | A | 194 | 37.945 | 63.143 | 138.128 | 1.00 | 0.00 | xxxx | 1519 |
| ATOM | 1520 | CE3 | TRP | A | 194 | 38.448 | 62.727 | 139.368 | 1.00 | 0.00 | xxxx | 1520 |
| ATOM | 1521 | CZ3 | TRP | A | 194 | 39.729 | 63.114 | 139.724 | 1.00 | 0.00 | xxxx | 1521 |
| ATOM | 1522 | CH2 | TRP | A | 194 | 40.505 | 63.930 | 138.887 | 1.00 | 0.00 | xxxx | 1522 |
| ATOM | 1523 | CZ2 | TRP | A | 194 | 40.037 | 64.356 | 137.671 | 1.00 | 0.00 | xxxx | 1523 |
| ATOM | 1524 | C | TRP | A | 194 | 33.194 | 61.363 | 137.577 | 1.00 | 0.00 | xxxx | 1524 |
| ATOM | 1525 | O | TRP | A | 194 | 33.000 | 60.453 | 136.773 | 1.00 | 0.00 | xxxx | 1525 |
| ATOM | 1526 | N | LEU | A | 195 | 32.539 | 61.445 | 138.734 | 1.00 | 0.00 | xxxx | 1526 |
| ATOM | 1527 | CA | LEU | A | 195 | 31.555 | 60.419 | 139.101 | 1.00 | 0.00 | xxxx | 1527 |
| ATOM | 1528 | CB | LEU | A | 195 | 31.105 | 60.591 | 140.555 | 1.00 | 0.00 | xxxx | 1528 |
| ATOM | 1529 | CG | LEU | A | 195 | 32.194 | 60.179 | 141.550 | 1.00 | 0.00 | xxxx | 1529 |
| ATOM | 1530 | CD1 | LEU | A | 195 | 31.930 | 60.776 | 142.929 | 1.00 | 0.00 | xxxx | 1530 |
| ATOM | 1531 | CD2 | LEU | A | 195 | 32.294 | 58.658 | 141.647 | 1.00 | 0.00 | xxxx | 1531 |
| ATOM | 1532 | C | LEU | A | 195 | 30.351 | 60.446 | 138.156 | 1.00 | 0.00 | xxxx | 1532 |
| ATOM | 1533 | O | LEU | A | 195 | 29.782 | 59.403 | 137.827 | 1.00 | 0.00 | xxxx | 1533 |
| ATOM | 1534 | N | SER | A | 196 | 29.971 | 61.638 | 137.717 | 1.00 | 0.00 | xxxx | 1534 |
| ATOM | 1535 | CA | SER | A | 196 | 28.892 | 61.757 | 136.749 | 1.00 | 0.00 | xxxx | 1535 |
| ATOM | 1536 | CB | SER | A | 196 | 28.511 | 63.223 | 136.553 | 1.00 | 0.00 | xxxx | 1536 |
| ATOM | 1537 | OG | SER | A | 196 | 27.466 | 63.342 | 135.607 | 1.00 | 0.00 | xxxx | 1537 |
| ATOM | 1538 | C | SER | A | 196 | 29.292 | 61.137 | 135.412 | 1.00 | 0.00 | xxxx | 1538 |
| ATOM | 1539 | O | SER | A | 196 | 28.492 | 60.464 | 134.759 | 1.00 | 0.00 | xxxx | 1539 |
| ATOM | 1540 | N | SER | A | 197 | 30.534 | 61.370 | 135.006 | 1.00 | 0.00 | xxxx | 1540 |
| ATOM | 1541 | CA | SER | A | 197 | 31.015 | 60.884 | 133.710 | 1.00 | 0.00 | xxxx | 1541 |
| ATOM | 1542 | CB | SER | A | 197 | 32.301 | 61.614 | 133.315 | 1.00 | 0.00 | xxxx | 1542 |
| ATOM | 1543 | OG | SER | A | 197 | 32.056 | 62.978 | 133.022 | 1.00 | 0.00 | xxxx | 1543 |
| ATOM | 1544 | C | SER | A | 197 | 31.278 | 59.380 | 133.665 | 1.00 | 0.00 | xxxx | 1544 |
| ATOM | 1545 | O | SER | A | 197 | 30.903 | 58.712 | 132.700 | 1.00 | 0.00 | xxxx | 1545 |
| ATOM | 1546 | N | PHE | A | 198 | 31.933 | 58.857 | 134.706 | 1.00 | 0.00 | xxxx | 1546 |
| ATOM | 1547 | CA | PHE | A | 198 | 32.506 | 57.513 | 134.659 | 1.00 | 0.00 | xxxx | 1547 |
| ATOM | 1548 | CB | PHE | A | 198 | 34.024 | 57.551 | 134.909 | 1.00 | 0.00 | xxxx | 1548 |
| ATOM | 1549 | CG | PHE | A | 198 | 34.792 | 58.382 | 133.926 | 1.00 | 0.00 | xxxx | 1549 |
| ATOM | 1550 | CD1 | PHE | A | 198 | 34.956 | 57.953 | 132.613 | 1.00 | 0.00 | xxxx | 1550 |
| ATOM | 1551 | CE1 | PHE | A | 198 | 35.672 | 58.712 | 131.702 | 1.00 | 0.00 | xxxx | 1551 |
| ATOM | 1552 | CZ | PHE | A | 198 | 36.244 | 59.908 | 132.104 | 1.00 | 0.00 | xxxx | 1552 |
| ATOM | 1553 | CE2 | PHE | A | 198 | 36.093 | 60.340 | 133.413 | 1.00 | 0.00 | xxxx | 1553 |
| ATOM | 1554 | CD2 | PHE | A | 198 | 35.374 | 59.576 | 134.314 | 1.00 | 0.00 | xxxx | 1554 |
| ATOM | 1555 | C | PHE | A | 198 | 31.877 | 56.591 | 135.681 | 1.00 | 0.00 | xxxx | 1555 |
| ATOM | 1556 | O | PHE | A | 198 | 31.911 | 55.370 | 135.529 | 1.00 | 0.00 | xxxx | 1556 |
| ATOM | 1557 | N | GLY | A | 199 | 31.334 | 57.179 | 136.741 | 1.00 | 0.00 | xxxx | 1557 |
| ATOM | 1558 | CA | GLY | A | 199 | 30.585 | 56.415 | 137.720 | 1.00 | 0.00 | xxxx | 1558 |
| ATOM | 1559 | C | GLY | A | 199 | 31.304 | 55.216 | 138.300 | 1.00 | 0.00 | xxxx | 1559 |
| ATOM | 1560 | O | GLY | A | 199 | 32.402 | 55.331 | 138.867 | 1.00 | 0.00 | xxxx | 1560 |
| ATOM | 1561 | N | ASP | A | 200 | 30.704 | 54.043 | 138.136 | 1.00 | 0.00 | xxxx | 1561 |
| ATOM | 1562 | C | ASP | A | 200 | 32.465 | 52.304 | 138.046 | 1.00 | 0.00 | xxxx | 1562 |
| ATOM | 1563 | O | ASP | A | 200 | 32.968 | 51.244 | 138.394 | 1.00 | 0.00 | xxxx | 1563 |
| ATOM | 1564 | CA | ASP | A | 200 | 31.249 | 52.859 | 138.788 | 1.00 | 0.00 | xxxx | 1564 |
| ATOM | 1565 | CB | ASP | A | 200 | 30.181 | 51.771 | 138.933 | 1.00 | 0.00 | xxxx | 1565 |
| ATOM | 1566 | CG | ASP | A | 200 | 30.510 | 50.778 | 140.040 | 1.00 | 0.00 | xxxx | 1566 |
| ATOM | 1567 | OD1 | ASP | A | 200 | 30.918 | 51.218 | 141.140 | 1.00 | 0.00 | xxxx | 1567 |
| ATOM | 1568 | OD2 | ASP | A | 200 | 30.372 | 49.560 | 139.805 | 1.00 | 0.00 | xxxx | 1568 |
| ATOM | 1569 | N | LYS | A | 201 | 32.957 | 53.030 | 137.045 | 1.00 | 0.00 | xxxx | 1569 |
| ATOM | 1570 | CA | LYS | A | 201 | 34.201 | 52.610 | 136.394 | 1.00 | 0.00 | xxxx | 1570 |
| ATOM | 1571 | CB | LYS | A | 201 | 34.411 | 53.325 | 135.062 | 1.00 | 0.00 | xxxx | 1571 |
| ATOM | 1572 | CG | LYS | A | 201 | 33.528 | 52.841 | 133.934 | 1.00 | 0.00 | xxxx | 1572 |
| ATOM | 1573 | CD | LYS | A | 201 | 33.955 | 53.489 | 132.626 | 1.00 | 0.00 | xxxx | 1573 |
| ATOM | 1574 | CE | LYS | A | 201 | 33.291 | 52.805 | 131.449 | 1.00 | 0.00 | xxxx | 1574 |
| ATOM | 1575 | NZ | LYS | A | 201 | 33.633 | 51.346 | 131.404 | 1.00 | 0.00 | xxxx | 1575 |
| ATOM | 1576 | C | LYS | A | 201 | 35.416 | 52.875 | 137.282 | 1.00 | 0.00 | xxxx | 1576 |
| ATOM | 1577 | O | LYS | A | 201 | 36.447 | 52.213 | 137.141 | 1.00 | 0.00 | xxxx | 1577 |
| ATOM | 1578 | N | ILE | A | 202 | 35.299 | 53.852 | 138.180 | 1.00 | 0.00 | xxxx | 1578 |
| ATOM | 1579 | CA | ILE | A | 202 | 36.416 | 54.249 | 139.025 | 1.00 | 0.00 | xxxx | 1579 |
| ATOM | 1580 | CB | ILE | A | 202 | 36.185 | 55.625 | 139.660 | 1.00 | 0.00 | xxxx | 1580 |
| ATOM | 1581 | CG1 | ILE | A | 202 | 35.829 | 56.664 | 138.591 | 1.00 | 0.00 | xxxx | 1581 |
| ATOM | 1582 | CD1 | ILE | A | 202 | 35.524 | 58.048 | 139.164 | 1.00 | 0.00 | xxxx | 1582 |
| ATOM | 1583 | CG2 | ILE | A | 202 | 37.425 | 56.056 | 140.438 | 1.00 | 0.00 | xxxx | 1583 |
| ATOM | 1584 | C | ILE | A | 202 | 36.651 | 53.204 | 140.107 | 1.00 | 0.00 | xxxx | 1584 |
| ATOM | 1585 | O | ILE | A | 202 | 35.737 | 52.884 | 140.865 | 1.00 | 0.00 | xxxx | 1585 |
| ATOM | 1586 | N | GLU | A | 203 | 37.878 | 52.694 | 140.188 | 1.00 | 0.00 | xxxx | 1586 |
| ATOM | 1587 | CA | GLU | A | 203 | 38.199 | 51.627 | 141.135 | 1.00 | 0.00 | xxxx | 1587 |
| ATOM | 1588 | CB | GLU | A | 203 | 38.778 | 50.417 | 140.384 | 1.00 | 0.00 | xxxx | 1588 |
| ATOM | 1589 | CG | GLU | A | 203 | 37.772 | 49.792 | 139.416 | 1.00 | 0.00 | xxxx | 1589 |
| ATOM | 1590 | CD | GLU | A | 203 | 38.353 | 48.684 | 138.547 | 1.00 | 0.00 | xxxx | 1590 |
| ATOM | 1591 | OE1 | GLU | A | 203 | 39.595 | 48.539 | 138.479 | 1.00 | 0.00 | xxxx | 1591 |
| ATOM | 1592 | OE2 | GLU | A | 203 | 37.561 | 47.942 | 137.920 | 1.00 | 0.00 | xxxx | 1592 |
| ATOM | 1593 | C | GLU | A | 203 | 39.158 | 52.075 | 142.233 | 1.00 | 0.00 | xxxx | 1593 |
| ATOM | 1594 | O | GLU | A | 203 | 39.313 | 51.390 | 143.245 | 1.00 | 0.00 | xxxx | 1594 |
| ATOM | 1595 | N | ALA | A | 204 | 39.778 | 53.235 | 142.051 | 1.00 | 0.00 | xxxx | 1595 |
| ATOM | 1596 | CA | ALA | A | 204 | 40.764 | 53.731 | 143.000 | 1.00 | 0.00 | xxxx | 1596 |
| ATOM | 1597 | CB | ALA | A | 204 | 42.099 | 53.007 | 142.809 | 1.00 | 0.00 | xxxx | 1597 |
| ATOM | 1598 | C | ALA | A | 204 | 40.944 | 55.236 | 142.834 | 1.00 | 0.00 | xxxx | 1598 |
| ATOM | 1599 | O | ALA | A | 204 | 40.877 | 55.761 | 141.720 | 1.00 | 0.00 | xxxx | 1599 |
| ATOM | 1600 | N | VAL | A | 205 | 41.167 | 55.934 | 143.945 | 1.00 | 0.00 | xxxx | 1600 |
| ATOM | 1601 | CA | VAL | A | 205 | 41.404 | 57.370 | 143.911 | 1.00 | 0.00 | xxxx | 1601 |
| ATOM | 1602 | CB | VAL | A | 205 | 40.278 | 58.154 | 144.615 | 1.00 | 0.00 | xxxx | 1602 |
| ATOM | 1603 | CG1 | VAL | A | 205 | 40.605 | 59.641 | 144.631 | 1.00 | 0.00 | xxxx | 1603 |
| ATOM | 1604 | CG2 | VAL | A | 205 | 38.935 | 57.899 | 143.941 | 1.00 | 0.00 | xxxx | 1604 |
| ATOM | 1605 | C | VAL | A | 205 | 42.724 | 57.692 | 144.578 | 1.00 | 0.00 | xxxx | 1605 |
| ATOM | 1606 | O | VAL | A | 205 | 42.915 | 57.366 | 145.749 | 1.00 | 0.00 | xxxx | 1606 |
| ATOM | 1607 | N | PHE | A | 206 | 43.621 | 58.331 | 143.835 | 1.00 | 0.00 | xxxx | 1607 |
| ATOM | 1608 | CA | PHE | A | 206 | 44.862 | 58.884 | 144.392 | 1.00 | 0.00 | xxxx | 1608 |
| ATOM | 1609 | CB | PHE | A | 206 | 46.082 | 58.571 | 143.509 | 1.00 | 0.00 | xxxx | 1609 |
| ATOM | 1610 | CG | PHE | A | 206 | 46.410 | 57.109 | 143.392 | 1.00 | 0.00 | xxxx | 1610 |
| ATOM | 1611 | CD1 | PHE | A | 206 | 47.402 | 56.542 | 144.172 | 1.00 | 0.00 | xxxx | 1611 |
| ATOM | 1612 | CE1 | PHE | A | 206 | 47.714 | 55.194 | 144.046 | 1.00 | 0.00 | xxxx | 1612 |
| ATOM | 1613 | CZ | PHE | A | 206 | 47.034 | 54.416 | 143.126 | 1.00 | 0.00 | xxxx | 1613 |
| ATOM | 1614 | CE2 | PHE | A | 206 | 46.037 | 54.971 | 142.339 | 1.00 | 0.00 | xxxx | 1614 |
| ATOM | 1615 | CD2 | PHE | A | 206 | 45.738 | 56.312 | 142.466 | 1.00 | 0.00 | xxxx | 1615 |
| ATOM | 1616 | C | PHE | A | 206 | 44.733 | 60.397 | 144.508 | 1.00 | 0.00 | xxxx | 1616 |
| ATOM | 1617 | O | PHE | A | 206 | 44.292 | 61.053 | 143.565 | 1.00 | 0.00 | xxxx | 1617 |
| ATOM | 1618 | N | ALA | A | 207 | 45.142 | 60.957 | 145.645 | 1.00 | 0.00 | xxxx | 1618 |
| ATOM | 1619 | CA | ALA | A | 207 | 45.225 | 62.416 | 145.769 | 1.00 | 0.00 | xxxx | 1619 |
| ATOM | 1620 | CB | ALA | A | 207 | 44.123 | 62.950 | 146.678 | 1.00 | 0.00 | xxxx | 1620 |
| ATOM | 1621 | C | ALA | A | 207 | 46.594 | 62.810 | 146.306 | 1.00 | 0.00 | xxxx | 1621 |
| ATOM | 1622 | O | ALA | A | 207 | 47.122 | 62.146 | 147.201 | 1.00 | 0.00 | xxxx | 1622 |
| ATOM | 1623 | N | ASN | A | 208 | 47.162 | 63.896 | 145.787 | 1.00 | 0.00 | xxxx | 1623 |
| ATOM | 1624 | CA | ASN | A | 208 | 48.502 | 64.291 | 146.219 | 1.00 | 0.00 | xxxx | 1624 |
| ATOM | 1625 | CB | ASN | A | 208 | 49.103 | 65.400 | 145.344 | 1.00 | 0.00 | xxxx | 1625 |
| ATOM | 1626 | CG | ASN | A | 208 | 49.459 | 64.984 | 143.909 | 1.00 | 0.00 | xxxx | 1626 |
| ATOM | 1627 | OD1 | ASN | A | 208 | 49.763 | 65.868 | 143.121 | 1.00 | 0.00 | xxxx | 1627 |
| ATOM | 1628 | ND2 | ASN | A | 208 | 49.449 | 63.689 | 143.571 | 1.00 | 0.00 | xxxx | 1628 |
| ATOM | 1629 | C | ASN | A | 208 | 48.517 | 64.796 | 147.679 | 1.00 | 0.00 | xxxx | 1629 |
| ATOM | 1630 | O | ASN | A | 208 | 49.590 | 64.903 | 148.269 | 1.00 | 0.00 | xxxx | 1630 |
| ATOM | 1631 | N | ASN | A | 209 | 47.365 | 65.153 | 148.253 | 1.00 | 0.00 | xxxx | 1631 |
| ATOM | 1632 | CA | ASN | A | 209 | 47.342 | 65.466 | 149.688 | 1.00 | 0.00 | xxxx | 1632 |
| ATOM | 1633 | CB | ASN | A | 209 | 47.691 | 66.955 | 149.969 | 1.00 | 0.00 | xxxx | 1633 |
| ATOM | 1634 | CG | ASN | A | 209 | 46.526 | 67.920 | 149.719 | 1.00 | 0.00 | xxxx | 1634 |
| ATOM | 1635 | OD1 | ASN | A | 209 | 45.537 | 67.573 | 149.075 | 1.00 | 0.00 | xxxx | 1635 |
| ATOM | 1636 | ND2 | ASN | A | 209 | 46.667 | 69.158 | 150.209 | 1.00 | 0.00 | xxxx | 1636 |
| ATOM | 1637 | C | ASN | A | 209 | 46.007 | 65.097 | 150.320 | 1.00 | 0.00 | xxxx | 1637 |
| ATOM | 1638 | O | ASN | A | 209 | 45.059 | 64.713 | 149.633 | 1.00 | 0.00 | xxxx | 1638 |
| ATOM | 1639 | N | ASP | A | 210 | 45.958 | 65.204 | 151.644 | 1.00 | 0.00 | xxxx | 1639 |
| ATOM | 1640 | CA | ASP | A | 210 | 44.773 | 64.808 | 152.391 | 1.00 | 0.00 | xxxx | 1640 |
| ATOM | 1641 | CB | ASP | A | 210 | 45.055 | 64.800 | 153.892 | 1.00 | 0.00 | xxxx | 1641 |
| ATOM | 1642 | CG | ASP | A | 210 | 45.798 | 63.549 | 154.341 | 1.00 | 0.00 | xxxx | 1642 |
| ATOM | 1643 | OD1 | ASP | A | 210 | 46.085 | 62.662 | 153.499 | 1.00 | 0.00 | xxxx | 1643 |
| ATOM | 1644 | OD2 | ASP | A | 210 | 46.092 | 63.456 | 155.547 | 1.00 | 0.00 | xxxx | 1644 |
| ATOM | 1645 | C | ASP | A | 210 | 43.581 | 65.701 | 152.100 | 1.00 | 0.00 | xxxx | 1645 |
| ATOM | 1646 | O | ASP | A | 210 | 42.472 | 65.208 | 151.978 | 1.00 | 0.00 | xxxx | 1646 |
| ATOM | 1647 | N | ASP | A | 211 | 43.791 | 67.008 | 151.985 | 1.00 | 0.00 | xxxx | 1647 |
| ATOM | 1648 | CA | ASP | A | 211 | 42.638 | 67.877 | 151.766 | 1.00 | 0.00 | xxxx | 1648 |
| ATOM | 1649 | CB | ASP | A | 211 | 43.052 | 69.350 | 151.760 | 1.00 | 0.00 | xxxx | 1649 |
| ATOM | 1650 | CG | ASP | A | 211 | 42.782 | 70.029 | 153.091 | 1.00 | 0.00 | xxxx | 1650 |
| ATOM | 1651 | OD1 | ASP | A | 211 | 42.301 | 69.349 | 154.028 | 1.00 | 0.00 | xxxx | 1651 |
| ATOM | 1652 | OD2 | ASP | A | 211 | 43.055 | 71.240 | 153.210 | 1.00 | 0.00 | xxxx | 1652 |
| ATOM | 1653 | C | ASP | A | 211 | 41.921 | 67.506 | 150.469 | 1.00 | 0.00 | xxxx | 1653 |
| ATOM | 1654 | O | ASP | A | 211 | 40.698 | 67.469 | 150.421 | 1.00 | 0.00 | xxxx | 1654 |
| ATOM | 1655 | N | MET | A | 212 | 42.680 | 67.193 | 149.427 | 1.00 | 0.00 | xxxx | 1655 |
| ATOM | 1656 | CA | MET | A | 212 | 42.048 | 66.791 | 148.177 | 1.00 | 0.00 | xxxx | 1656 |
| ATOM | 1657 | CB | MET | A | 212 | 43.069 | 66.817 | 147.034 | 1.00 | 0.00 | xxxx | 1657 |
| ATOM | 1658 | CG | MET | A | 212 | 43.477 | 68.258 | 146.686 | 1.00 | 0.00 | xxxx | 1658 |
| ATOM | 1659 | SD | MET | A | 212 | 44.495 | 68.384 | 145.210 | 1.00 | 0.00 | xxxx | 1659 |
| ATOM | 1660 | CE | MET | A | 212 | 45.912 | 67.363 | 145.682 | 1.00 | 0.00 | xxxx | 1660 |
| ATOM | 1661 | C | MET | A | 212 | 41.397 | 65.419 | 148.310 | 1.00 | 0.00 | xxxx | 1661 |
| ATOM | 1662 | O | MET | A | 212 | 40.318 | 65.194 | 147.763 | 1.00 | 0.00 | xxxx | 1662 |
| ATOM | 1663 | N | ALA | A | 213 | 42.031 | 64.506 | 149.047 | 1.00 | 0.00 | xxxx | 1663 |
| ATOM | 1664 | CA | ALA | A | 213 | 41.423 | 63.195 | 149.279 | 1.00 | 0.00 | xxxx | 1664 |
| ATOM | 1665 | CB | ALA | A | 213 | 42.346 | 62.289 | 150.078 | 1.00 | 0.00 | xxxx | 1665 |
| ATOM | 1666 | C | ALA | A | 213 | 40.094 | 63.354 | 150.002 | 1.00 | 0.00 | xxxx | 1666 |
| ATOM | 1667 | O | ALA | A | 213 | 39.126 | 62.658 | 149.683 | 1.00 | 0.00 | xxxx | 1667 |
| ATOM | 1668 | N | LEU | A | 214 | 40.048 | 64.282 | 150.964 | 1.00 | 0.00 | xxxx | 1668 |
| ATOM | 1669 | CA | LEU | A | 214 | 38.842 | 64.516 | 151.759 | 1.00 | 0.00 | xxxx | 1669 |
| ATOM | 1670 | CB | LEU | A | 214 | 39.158 | 65.434 | 152.947 | 1.00 | 0.00 | xxxx | 1670 |
| ATOM | 1671 | CG | LEU | A | 214 | 40.111 | 64.799 | 153.966 | 1.00 | 0.00 | xxxx | 1671 |
| ATOM | 1672 | CD1 | LEU | A | 214 | 40.612 | 65.821 | 154.986 | 1.00 | 0.00 | xxxx | 1672 |
| ATOM | 1673 | CD2 | LEU | A | 214 | 39.464 | 63.609 | 154.670 | 1.00 | 0.00 | xxxx | 1673 |
| ATOM | 1674 | C | LEU | A | 214 | 37.717 | 65.104 | 150.922 | 1.00 | 0.00 | xxxx | 1674 |
| ATOM | 1675 | O | LEU | A | 214 | 36.555 | 64.746 | 151.102 | 1.00 | 0.00 | xxxx | 1675 |
| ATOM | 1676 | N | GLY | A | 215 | 38.066 | 66.001 | 150.004 | 1.00 | 0.00 | xxxx | 1676 |
| ATOM | 1677 | CA | GLY | A | 215 | 37.088 | 66.537 | 149.067 | 1.00 | 0.00 | xxxx | 1677 |
| ATOM | 1678 | C | GLY | A | 215 | 36.551 | 65.438 | 148.168 | 1.00 | 0.00 | xxxx | 1678 |
| ATOM | 1679 | O | GLY | A | 215 | 35.345 | 65.377 | 147.911 | 1.00 | 0.00 | xxxx | 1679 |
| ATOM | 1680 | N | ALA | A | 216 | 37.442 | 64.573 | 147.688 | 1.00 | 0.00 | xxxx | 1680 |
| ATOM | 1681 | CA | ALA | A | 216 | 37.020 | 63.429 | 146.885 | 1.00 | 0.00 | xxxx | 1681 |
| ATOM | 1682 | CB | ALA | A | 216 | 38.230 | 62.655 | 146.374 | 1.00 | 0.00 | xxxx | 1682 |
| ATOM | 1683 | C | ALA | A | 216 | 36.090 | 62.516 | 147.688 | 1.00 | 0.00 | xxxx | 1683 |
| ATOM | 1684 | O | ALA | A | 216 | 35.066 | 62.062 | 147.170 | 1.00 | 0.00 | xxxx | 1684 |
| ATOM | 1685 | N | ILE | A | 217 | 36.424 | 62.270 | 148.952 | 1.00 | 0.00 | xxxx | 1685 |
| ATOM | 1686 | CA | ILE | A | 217 | 35.583 | 61.438 | 149.805 | 1.00 | 0.00 | xxxx | 1686 |
| ATOM | 1687 | CB | ILE | A | 217 | 36.275 | 61.186 | 151.167 | 1.00 | 0.00 | xxxx | 1687 |
| ATOM | 1688 | CG1 | ILE | A | 217 | 37.406 | 60.171 | 150.988 | 1.00 | 0.00 | xxxx | 1688 |
| ATOM | 1689 | CD1 | ILE | A | 217 | 38.425 | 60.184 | 152.118 | 1.00 | 0.00 | xxxx | 1689 |
| ATOM | 1690 | CG2 | ILE | A | 217 | 35.265 | 60.695 | 152.221 | 1.00 | 0.00 | xxxx | 1690 |
| ATOM | 1691 | C | ILE | A | 217 | 34.187 | 62.049 | 149.971 | 1.00 | 0.00 | xxxx | 1691 |
| ATOM | 1692 | O | ILE | A | 217 | 33.188 | 61.327 | 149.967 | 1.00 | 0.00 | xxxx | 1692 |
| ATOM | 1693 | N | GLU | A | 218 | 34.089 | 63.371 | 150.075 | 1.00 | 0.00 | xxxx | 1693 |
| ATOM | 1694 | C | GLU | A | 218 | 31.948 | 63.832 | 148.945 | 1.00 | 0.00 | xxxx | 1694 |
| ATOM | 1695 | O | GLU | A | 218 | 30.736 | 63.621 | 149.019 | 1.00 | 0.00 | xxxx | 1695 |
| ATOM | 1696 | CA | GLU | A | 218 | 32.781 | 64.001 | 150.218 | 1.00 | 0.00 | xxxx | 1696 |
| ATOM | 1697 | CB | GLU | A | 218 | 32.925 | 65.482 | 150.574 | 1.00 | 0.00 | xxxx | 1697 |
| ATOM | 1698 | CG | GLU | A | 218 | 33.457 | 65.727 | 151.983 | 1.00 | 0.00 | xxxx | 1698 |
| ATOM | 1699 | CD | GLU | A | 218 | 32.729 | 64.911 | 153.046 | 1.00 | 0.00 | xxxx | 1699 |
| ATOM | 1700 | OE1 | GLU | A | 218 | 31.482 | 64.948 | 153.089 | 1.00 | 0.00 | xxxx | 1700 |
| ATOM | 1701 | OE2 | GLU | A | 218 | 33.401 | 64.221 | 153.840 | 1.00 | 0.00 | xxxx | 1701 |
| ATOM | 1702 | N | ALA | A | 219 | 32.593 | 63.921 | 147.783 | 1.00 | 0.00 | xxxx | 1702 |
| ATOM | 1703 | CA | ALA | A | 219 | 31.881 | 63.696 | 146.531 | 1.00 | 0.00 | xxxx | 1703 |
| ATOM | 1704 | CB | ALA | A | 219 | 32.763 | 64.063 | 145.327 | 1.00 | 0.00 | xxxx | 1704 |
| ATOM | 1705 | C | ALA | A | 219 | 31.431 | 62.237 | 146.444 | 1.00 | 0.00 | xxxx | 1705 |
| ATOM | 1706 | O | ALA | A | 219 | 30.321 | 61.945 | 145.997 | 1.00 | 0.00 | xxxx | 1706 |
| ATOM | 1707 | N | LEU | A | 220 | 32.294 | 61.329 | 146.890 | 1.00 | 0.00 | xxxx | 1707 |
| ATOM | 1708 | CA | LEU | A | 220 | 31.957 | 59.907 | 146.898 | 1.00 | 0.00 | xxxx | 1708 |
| ATOM | 1709 | CB | LEU | A | 220 | 33.175 | 59.083 | 147.291 | 1.00 | 0.00 | xxxx | 1709 |
| ATOM | 1710 | CG | LEU | A | 220 | 34.251 | 59.007 | 146.209 | 1.00 | 0.00 | xxxx | 1710 |
| ATOM | 1711 | CD1 | LEU | A | 220 | 35.601 | 58.672 | 146.812 | 1.00 | 0.00 | xxxx | 1711 |
| ATOM | 1712 | CD2 | LEU | A | 220 | 33.837 | 57.986 | 145.144 | 1.00 | 0.00 | xxxx | 1712 |
| ATOM | 1713 | C | LEU | A | 220 | 30.793 | 59.605 | 147.836 | 1.00 | 0.00 | xxxx | 1713 |
| ATOM | 1714 | O | LEU | A | 220 | 29.876 | 58.868 | 147.475 | 1.00 | 0.00 | xxxx | 1714 |
| ATOM | 1715 | N | LYS | A | 221 | 30.824 | 60.164 | 149.045 | 1.00 | 0.00 | xxxx | 1715 |
| ATOM | 1716 | CA | LYS | A | 221 | 29.687 | 60.015 | 149.962 | 1.00 | 0.00 | xxxx | 1716 |
| ATOM | 1717 | CB | LYS | A | 221 | 29.930 | 60.789 | 151.258 | 1.00 | 0.00 | xxxx | 1717 |
| ATOM | 1718 | CG | LYS | A | 221 | 30.943 | 60.170 | 152.186 | 1.00 | 0.00 | xxxx | 1718 |
| ATOM | 1719 | CD | LYS | A | 221 | 31.170 | 61.087 | 153.369 | 1.00 | 0.00 | xxxx | 1719 |
| ATOM | 1720 | CE | LYS | A | 221 | 32.082 | 60.452 | 154.396 | 1.00 | 0.00 | xxxx | 1720 |
| ATOM | 1721 | NZ | LYS | A | 221 | 32.368 | 61.404 | 155.504 | 1.00 | 0.00 | xxxx | 1721 |
| ATOM | 1722 | C | LYS | A | 221 | 28.379 | 60.492 | 149.320 | 1.00 | 0.00 | xxxx | 1722 |
| ATOM | 1723 | O | LYS | A | 221 | 27.353 | 59.801 | 149.365 | 1.00 | 0.00 | xxxx | 1723 |
| ATOM | 1724 | N | SER | A | 222 | 28.431 | 61.671 | 148.709 | 1.00 | 0.00 | xxxx | 1724 |
| ATOM | 1725 | CA | SER | A | 222 | 27.258 | 62.263 | 148.063 | 1.00 | 0.00 | xxxx | 1725 |
| ATOM | 1726 | CB | SER | A | 222 | 27.626 | 63.620 | 147.450 | 1.00 | 0.00 | xxxx | 1726 |
| ATOM | 1727 | OG | SER | A | 222 | 26.484 | 64.308 | 146.967 | 1.00 | 0.00 | xxxx | 1727 |
| ATOM | 1728 | C | SER | A | 222 | 26.670 | 61.340 | 146.992 | 1.00 | 0.00 | xxxx | 1728 |
| ATOM | 1729 | O | SER | A | 222 | 25.451 | 61.321 | 146.773 | 1.00 | 0.00 | xxxx | 1729 |
| ATOM | 1730 | N | ALA | A | 223 | 27.539 | 60.569 | 146.346 | 1.00 | 0.00 | xxxx | 1730 |
| ATOM | 1731 | CA | ALA | A | 223 | 27.144 | 59.633 | 145.295 | 1.00 | 0.00 | xxxx | 1731 |
| ATOM | 1732 | CB | ALA | A | 223 | 28.227 | 59.565 | 144.226 | 1.00 | 0.00 | xxxx | 1732 |
| ATOM | 1733 | C | ALA | A | 223 | 26.844 | 58.224 | 145.823 | 1.00 | 0.00 | xxxx | 1733 |
| ATOM | 1734 | O | ALA | A | 223 | 26.640 | 57.299 | 145.039 | 1.00 | 0.00 | xxxx | 1734 |
| ATOM | 1735 | N | GLY | A | 224 | 26.837 | 58.055 | 147.143 | 1.00 | 0.00 | xxxx | 1735 |
| ATOM | 1736 | CA | GLY | A | 224 | 26.391 | 56.803 | 147.737 | 1.00 | 0.00 | xxxx | 1736 |
| ATOM | 1737 | C | GLY | A | 224 | 27.439 | 55.849 | 148.286 | 1.00 | 0.00 | xxxx | 1737 |
| ATOM | 1738 | O | GLY | A | 224 | 27.099 | 54.754 | 148.749 | 1.00 | 0.00 | xxxx | 1738 |
| ATOM | 1739 | N | TYR | A | 225 | 28.705 | 56.254 | 148.247 | 1.00 | 0.00 | xxxx | 1739 |
| ATOM | 1740 | CA | TYR | A | 225 | 29.790 | 55.423 | 148.772 | 1.00 | 0.00 | xxxx | 1740 |
| ATOM | 1741 | CB | TYR | A | 225 | 31.126 | 55.823 | 148.132 | 1.00 | 0.00 | xxxx | 1741 |
| ATOM | 1742 | CG | TYR | A | 225 | 31.231 | 55.438 | 146.673 | 1.00 | 0.00 | xxxx | 1742 |
| ATOM | 1743 | CD1 | TYR | A | 225 | 30.582 | 56.178 | 145.691 | 1.00 | 0.00 | xxxx | 1743 |
| ATOM | 1744 | CE1 | TYR | A | 225 | 30.666 | 55.826 | 144.354 | 1.00 | 0.00 | xxxx | 1744 |
| ATOM | 1745 | CZ | TYR | A | 225 | 31.403 | 54.721 | 143.983 | 1.00 | 0.00 | xxxx | 1745 |
| ATOM | 1746 | OH | TYR | A | 225 | 31.483 | 54.376 | 142.652 | 1.00 | 0.00 | xxxx | 1746 |
| ATOM | 1747 | CE2 | TYR | A | 225 | 32.056 | 53.963 | 144.937 | 1.00 | 0.00 | xxxx | 1747 |
| ATOM | 1748 | CD2 | TYR | A | 225 | 31.963 | 54.321 | 146.276 | 1.00 | 0.00 | xxxx | 1748 |
| ATOM | 1749 | C | TYR | A | 225 | 29.901 | 55.507 | 150.290 | 1.00 | 0.00 | xxxx | 1749 |
| ATOM | 1750 | O | TYR | A | 225 | 29.427 | 56.464 | 150.907 | 1.00 | 0.00 | xxxx | 1750 |
| ATOM | 1751 | N | PHE | A | 226 | 30.514 | 54.479 | 150.874 | 1.00 | 0.00 | xxxx | 1751 |
| ATOM | 1752 | CA | PHE | A | 226 | 30.855 | 54.433 | 152.295 | 1.00 | 0.00 | xxxx | 1752 |
| ATOM | 1753 | CB | PHE | A | 226 | 31.676 | 55.662 | 152.679 | 1.00 | 0.00 | xxxx | 1753 |
| ATOM | 1754 | CG | PHE | A | 226 | 32.925 | 55.809 | 151.866 | 1.00 | 0.00 | xxxx | 1754 |
| ATOM | 1755 | CD1 | PHE | A | 226 | 33.835 | 54.764 | 151.787 | 1.00 | 0.00 | xxxx | 1755 |
| ATOM | 1756 | CE1 | PHE | A | 226 | 34.990 | 54.881 | 151.019 | 1.00 | 0.00 | xxxx | 1756 |
| ATOM | 1757 | CZ | PHE | A | 226 | 35.233 | 56.055 | 150.313 | 1.00 | 0.00 | xxxx | 1757 |
| ATOM | 1758 | CE2 | PHE | A | 226 | 34.318 | 57.101 | 150.383 | 1.00 | 0.00 | xxxx | 1758 |
| ATOM | 1759 | CD2 | PHE | A | 226 | 33.174 | 56.972 | 151.150 | 1.00 | 0.00 | xxxx | 1759 |
| ATOM | 1760 | C | PHE | A | 226 | 29.627 | 54.297 | 153.184 | 1.00 | 0.00 | xxxx | 1760 |
| ATOM | 1761 | O | PHE | A | 226 | 29.662 | 54.619 | 154.369 | 1.00 | 0.00 | xxxx | 1761 |
| ATOM | 1762 | N | THR | A | 227 | 28.547 | 53.815 | 152.583 | 1.00 | 0.00 | xxxx | 1762 |
| ATOM | 1763 | CA | THR | A | 227 | 27.374 | 53.347 | 153.311 | 1.00 | 0.00 | xxxx | 1763 |
| ATOM | 1764 | CB | THR | A | 227 | 26.239 | 54.382 | 153.335 | 1.00 | 0.00 | xxxx | 1764 |
| ATOM | 1765 | OG1 | THR | A | 227 | 25.133 | 53.854 | 154.076 | 1.00 | 0.00 | xxxx | 1765 |
| ATOM | 1766 | CG2 | THR | A | 227 | 25.785 | 54.714 | 151.924 | 1.00 | 0.00 | xxxx | 1766 |
| ATOM | 1767 | C | THR | A | 227 | 26.930 | 52.070 | 152.610 | 1.00 | 0.00 | xxxx | 1767 |
| ATOM | 1768 | O | THR | A | 227 | 27.137 | 51.920 | 151.405 | 1.00 | 0.00 | xxxx | 1768 |
| ATOM | 1769 | N | GLY | A | 228 | 26.331 | 51.145 | 153.349 | 1.00 | 0.00 | xxxx | 1769 |
| ATOM | 1770 | CA | GLY | A | 228 | 26.122 | 49.818 | 152.804 | 1.00 | 0.00 | xxxx | 1770 |
| ATOM | 1771 | C | GLY | A | 228 | 27.484 | 49.199 | 152.547 | 1.00 | 0.00 | xxxx | 1771 |
| ATOM | 1772 | O | GLY | A | 228 | 28.433 | 49.473 | 153.281 | 1.00 | 0.00 | xxxx | 1772 |
| ATOM | 1773 | N | ASN | A | 229 | 27.601 | 48.384 | 151.505 | 1.00 | 0.00 | xxxx | 1773 |
| ATOM | 1774 | CA | ASN | A | 229 | 28.884 | 47.756 | 151.200 | 1.00 | 0.00 | xxxx | 1774 |
| ATOM | 1775 | CB | ASN | A | 229 | 28.697 | 46.259 | 150.932 | 1.00 | 0.00 | xxxx | 1775 |
| ATOM | 1776 | CG | ASN | A | 229 | 27.629 | 45.977 | 149.888 | 1.00 | 0.00 | xxxx | 1776 |
| ATOM | 1777 | OD1 | ASN | A | 229 | 27.160 | 46.885 | 149.199 | 1.00 | 0.00 | xxxx | 1777 |
| ATOM | 1778 | ND2 | ASN | A | 229 | 27.241 | 44.711 | 149.766 | 1.00 | 0.00 | xxxx | 1778 |
| ATOM | 1779 | C | ASN | A | 229 | 29.591 | 48.414 | 150.013 | 1.00 | 0.00 | xxxx | 1779 |
| ATOM | 1780 | O | ASN | A | 229 | 30.509 | 47.831 | 149.435 | 1.00 | 0.00 | xxxx | 1780 |
| ATOM | 1781 | N | LYS | A | 230 | 29.165 | 49.627 | 149.661 | 1.00 | 0.00 | xxxx | 1781 |
| ATOM | 1782 | CA | LYS | A | 230 | 29.696 | 50.331 | 148.491 | 1.00 | 0.00 | xxxx | 1782 |
| ATOM | 1783 | CB | LYS | A | 230 | 28.645 | 51.284 | 147.926 | 1.00 | 0.00 | xxxx | 1783 |
| ATOM | 1784 | CG | LYS | A | 230 | 29.049 | 51.957 | 146.625 | 1.00 | 0.00 | xxxx | 1784 |
| ATOM | 1785 | CD | LYS | A | 230 | 27.920 | 52.840 | 146.108 | 1.00 | 0.00 | xxxx | 1785 |
| ATOM | 1786 | CE | LYS | A | 230 | 28.136 | 53.249 | 144.662 | 1.00 | 0.00 | xxxx | 1786 |
| ATOM | 1787 | NZ | LYS | A | 230 | 27.043 | 54.140 | 144.184 | 1.00 | 0.00 | xxxx | 1787 |
| ATOM | 1788 | C | LYS | A | 230 | 30.975 | 51.104 | 148.820 | 1.00 | 0.00 | xxxx | 1788 |
| ATOM | 1789 | O | LYS | A | 230 | 30.931 | 52.156 | 149.455 | 1.00 | 0.00 | xxxx | 1789 |
| ATOM | 1790 | N | TYR | A | 231 | 32.107 | 50.592 | 148.352 | 1.00 | 0.00 | xxxx | 1790 |
| ATOM | 1791 | CA | TYR | A | 231 | 33.403 | 51.068 | 148.820 | 1.00 | 0.00 | xxxx | 1791 |
| ATOM | 1792 | CB | TYR | A | 231 | 34.020 | 50.021 | 149.742 | 1.00 | 0.00 | xxxx | 1792 |
| ATOM | 1793 | CG | TYR | A | 231 | 35.396 | 50.366 | 150.248 | 1.00 | 0.00 | xxxx | 1793 |
| ATOM | 1794 | CD1 | TYR | A | 231 | 35.558 | 51.179 | 151.358 | 1.00 | 0.00 | xxxx | 1794 |
| ATOM | 1795 | CE1 | TYR | A | 231 | 36.809 | 51.500 | 151.828 | 1.00 | 0.00 | xxxx | 1795 |
| ATOM | 1796 | CZ | TYR | A | 231 | 37.927 | 50.995 | 151.187 | 1.00 | 0.00 | xxxx | 1796 |
| ATOM | 1797 | OH | TYR | A | 231 | 39.183 | 51.307 | 151.656 | 1.00 | 0.00 | xxxx | 1797 |
| ATOM | 1798 | CE2 | TYR | A | 231 | 37.790 | 50.178 | 150.084 | 1.00 | 0.00 | xxxx | 1798 |
| ATOM | 1799 | CD2 | TYR | A | 231 | 36.533 | 49.867 | 149.624 | 1.00 | 0.00 | xxxx | 1799 |
| ATOM | 1800 | C | TYR | A | 231 | 34.359 | 51.383 | 147.680 | 1.00 | 0.00 | xxxx | 1800 |
| ATOM | 1801 | O | TYR | A | 231 | 34.282 | 50.778 | 146.616 | 1.00 | 0.00 | xxxx | 1801 |
| ATOM | 1802 | N | ILE | A | 232 | 35.259 | 52.334 | 147.914 | 1.00 | 0.00 | xxxx | 1802 |
| ATOM | 1803 | CA | ILE | A | 232 | 36.330 | 52.633 | 146.976 | 1.00 | 0.00 | xxxx | 1803 |
| ATOM | 1804 | CB | ILE | A | 232 | 35.913 | 53.708 | 145.951 | 1.00 | 0.00 | xxxx | 1804 |
| ATOM | 1805 | CG1 | ILE | A | 232 | 37.027 | 53.945 | 144.941 | 1.00 | 0.00 | xxxx | 1805 |
| ATOM | 1806 | CD1 | ILE | A | 232 | 36.591 | 54.744 | 143.743 | 1.00 | 0.00 | xxxx | 1806 |
| ATOM | 1807 | CG2 | ILE | A | 232 | 35.555 | 55.020 | 146.649 | 1.00 | 0.00 | xxxx | 1807 |
| ATOM | 1808 | C | ILE | A | 232 | 37.548 | 53.063 | 147.793 | 1.00 | 0.00 | xxxx | 1808 |
| ATOM | 1809 | O | ILE | A | 232 | 37.413 | 53.817 | 148.754 | 1.00 | 0.00 | xxxx | 1809 |
| ATOM | 1810 | N | PRO | A | 233 | 38.739 | 52.558 | 147.441 | 1.00 | 0.00 | xxxx | 1810 |
| ATOM | 1811 | CA | PRO | A | 233 | 39.939 | 52.965 | 148.184 | 1.00 | 0.00 | xxxx | 1811 |
| ATOM | 1812 | CB | PRO | A | 233 | 40.955 | 51.880 | 147.820 | 1.00 | 0.00 | xxxx | 1812 |
| ATOM | 1813 | CG | PRO | A | 233 | 40.537 | 51.453 | 146.432 | 1.00 | 0.00 | xxxx | 1813 |
| ATOM | 1814 | CD | PRO | A | 233 | 39.026 | 51.487 | 146.470 | 1.00 | 0.00 | xxxx | 1814 |
| ATOM | 1815 | C | PRO | A | 233 | 40.438 | 54.344 | 147.769 | 1.00 | 0.00 | xxxx | 1815 |
| ATOM | 1816 | O | PRO | A | 233 | 40.593 | 54.627 | 146.578 | 1.00 | 0.00 | xxxx | 1816 |
| ATOM | 1817 | N | VAL | A | 234 | 40.678 | 55.196 | 148.762 | 1.00 | 0.00 | xxxx | 1817 |
| ATOM | 1818 | CA | VAL | A | 234 | 41.174 | 56.546 | 148.544 | 1.00 | 0.00 | xxxx | 1818 |
| ATOM | 1819 | CB | VAL | A | 234 | 40.142 | 57.602 | 148.976 | 1.00 | 0.00 | xxxx | 1819 |
| ATOM | 1820 | CG1 | VAL | A | 234 | 40.678 | 58.996 | 148.715 | 1.00 | 0.00 | xxxx | 1820 |
| ATOM | 1821 | CG2 | VAL | A | 234 | 38.804 | 57.384 | 148.267 | 1.00 | 0.00 | xxxx | 1821 |
| ATOM | 1822 | C | VAL | A | 234 | 42.452 | 56.709 | 149.345 | 1.00 | 0.00 | xxxx | 1822 |
| ATOM | 1823 | O | VAL | A | 234 | 42.484 | 56.346 | 150.522 | 1.00 | 0.00 | xxxx | 1823 |
| ATOM | 1824 | N | VAL | A | 235 | 43.501 | 57.258 | 148.732 | 1.00 | 0.00 | xxxx | 1824 |
| ATOM | 1825 | CA | VAL | A | 235 | 44.757 | 57.491 | 149.447 | 1.00 | 0.00 | xxxx | 1825 |
| ATOM | 1826 | CB | VAL | A | 235 | 45.869 | 56.539 | 148.964 | 1.00 | 0.00 | xxxx | 1826 |
| ATOM | 1827 | CG1 | VAL | A | 235 | 45.519 | 55.097 | 149.324 | 1.00 | 0.00 | xxxx | 1827 |
| ATOM | 1828 | CG2 | VAL | A | 235 | 46.099 | 56.699 | 147.468 | 1.00 | 0.00 | xxxx | 1828 |
| ATOM | 1829 | C | VAL | A | 235 | 45.189 | 58.952 | 149.316 | 1.00 | 0.00 | xxxx | 1829 |
| ATOM | 1830 | O | VAL | A | 235 | 44.972 | 59.583 | 148.280 | 1.00 | 0.00 | xxxx | 1830 |
| ATOM | 1831 | N | GLY | A | 236 | 45.764 | 59.496 | 150.387 | 1.00 | 0.00 | xxxx | 1831 |
| ATOM | 1832 | CA | GLY | A | 236 | 46.282 | 60.856 | 150.371 | 1.00 | 0.00 | xxxx | 1832 |
| ATOM | 1833 | C | GLY | A | 236 | 47.705 | 60.961 | 150.892 | 1.00 | 0.00 | xxxx | 1833 |
| ATOM | 1834 | O | GLY | A | 236 | 48.418 | 59.958 | 150.963 | 1.00 | 0.00 | xxxx | 1834 |
| ATOM | 1835 | N | VAL | A | 237 | 48.119 | 62.185 | 151.231 | 1.00 | 0.00 | xxxx | 1835 |
| ATOM | 1836 | CA | VAL | A | 237 | 49.399 | 62.461 | 151.893 | 1.00 | 0.00 | xxxx | 1836 |
| ATOM | 1837 | CB | VAL | A | 237 | 50.527 | 62.876 | 150.914 | 1.00 | 0.00 | xxxx | 1837 |
| ATOM | 1838 | CG1 | VAL | A | 237 | 51.746 | 63.309 | 151.700 | 1.00 | 0.00 | xxxx | 1838 |
| ATOM | 1839 | CG2 | VAL | A | 237 | 50.896 | 61.745 | 149.967 | 1.00 | 0.00 | xxxx | 1839 |
| ATOM | 1840 | C | VAL | A | 237 | 49.189 | 63.600 | 152.885 | 1.00 | 0.00 | xxxx | 1840 |
| ATOM | 1841 | O | VAL | A | 237 | 48.552 | 64.589 | 152.520 | 1.00 | 0.00 | xxxx | 1841 |
| ATOM | 1842 | N | ASP | A | 238 | 49.692 | 63.425 | 154.113 | 1.00 | 0.00 | xxxx | 1842 |
| ATOM | 1843 | CA | ASP | A | 238 | 49.966 | 64.468 | 155.127 | 1.00 | 0.00 | xxxx | 1843 |
| ATOM | 1844 | CB | ASP | A | 238 | 49.128 | 65.746 | 154.971 | 1.00 | 0.00 | xxxx | 1844 |
| ATOM | 1845 | CG | ASP | A | 238 | 49.636 | 66.668 | 153.871 | 1.00 | 0.00 | xxxx | 1845 |
| ATOM | 1846 | OD1 | ASP | A | 238 | 50.792 | 66.524 | 153.414 | 1.00 | 0.00 | xxxx | 1846 |
| ATOM | 1847 | OD2 | ASP | A | 238 | 48.841 | 67.527 | 153.432 | 1.00 | 0.00 | xxxx | 1847 |
| ATOM | 1848 | C | ASP | A | 238 | 49.713 | 63.921 | 156.526 | 1.00 | 0.00 | xxxx | 1848 |
| ATOM | 1849 | O | ASP | A | 238 | 50.480 | 64.184 | 157.447 | 1.00 | 0.00 | xxxx | 1849 |
| ATOM | 1850 | N | ALA | A | 239 | 48.611 | 63.191 | 156.679 | 1.00 | 0.00 | xxxx | 1850 |
| ATOM | 1851 | CA | ALA | A | 239 | 48.125 | 62.740 | 157.989 | 1.00 | 0.00 | xxxx | 1851 |
| ATOM | 1852 | CB | ALA | A | 239 | 49.145 | 61.812 | 158.674 | 1.00 | 0.00 | xxxx | 1852 |
| ATOM | 1853 | C | ALA | A | 239 | 47.783 | 63.936 | 158.881 | 1.00 | 0.00 | xxxx | 1853 |
| ATOM | 1854 | O | ALA | A | 239 | 48.171 | 64.008 | 160.055 | 1.00 | 0.00 | xxxx | 1854 |
| ATOM | 1855 | N | THR | A | 240 | 47.049 | 64.883 | 158.301 | 1.00 | 0.00 | xxxx | 1855 |
| ATOM | 1856 | CA | THR | A | 240 | 46.483 | 65.989 | 159.060 | 1.00 | 0.00 | xxxx | 1856 |
| ATOM | 1857 | CB | THR | A | 240 | 45.850 | 67.040 | 158.148 | 1.00 | 0.00 | xxxx | 1857 |
| ATOM | 1858 | OG1 | THR | A | 240 | 44.789 | 66.424 | 157.409 | 1.00 | 0.00 | xxxx | 1858 |
| ATOM | 1859 | CG2 | THR | A | 240 | 46.883 | 67.627 | 157.184 | 1.00 | 0.00 | xxxx | 1859 |
| ATOM | 1860 | C | THR | A | 240 | 45.408 | 65.453 | 159.994 | 1.00 | 0.00 | xxxx | 1860 |
| ATOM | 1861 | O | THR | A | 240 | 45.001 | 64.297 | 159.883 | 1.00 | 0.00 | xxxx | 1861 |
| ATOM | 1862 | N | ALA | A | 241 | 44.948 | 66.295 | 160.914 | 1.00 | 0.00 | xxxx | 1862 |
| ATOM | 1863 | CA | ALA | A | 241 | 43.896 | 65.887 | 161.837 | 1.00 | 0.00 | xxxx | 1863 |
| ATOM | 1864 | CB | ALA | A | 241 | 43.531 | 67.044 | 162.781 | 1.00 | 0.00 | xxxx | 1864 |
| ATOM | 1865 | C | ALA | A | 241 | 42.648 | 65.357 | 161.094 | 1.00 | 0.00 | xxxx | 1865 |
| ATOM | 1866 | O | ALA | A | 241 | 42.175 | 64.264 | 161.408 | 1.00 | 0.00 | xxxx | 1866 |
| ATOM | 1867 | N | PRO | A | 242 | 42.131 | 66.098 | 160.089 | 1.00 | 0.00 | xxxx | 1867 |
| ATOM | 1868 | CA | PRO | A | 242 | 40.978 | 65.500 | 159.401 | 1.00 | 0.00 | xxxx | 1868 |
| ATOM | 1869 | CB | PRO | A | 242 | 40.446 | 66.648 | 158.532 | 1.00 | 0.00 | xxxx | 1869 |
| ATOM | 1870 | CG | PRO | A | 242 | 41.594 | 67.555 | 158.345 | 1.00 | 0.00 | xxxx | 1870 |
| ATOM | 1871 | CD | PRO | A | 242 | 42.406 | 67.464 | 159.607 | 1.00 | 0.00 | xxxx | 1871 |
| ATOM | 1872 | C | PRO | A | 242 | 41.340 | 64.283 | 158.544 | 1.00 | 0.00 | xxxx | 1872 |
| ATOM | 1873 | O | PRO | A | 242 | 40.493 | 63.415 | 158.346 | 1.00 | 0.00 | xxxx | 1873 |
| ATOM | 1874 | N | GLY | A | 243 | 42.577 | 64.202 | 158.063 | 1.00 | 0.00 | xxxx | 1874 |
| ATOM | 1875 | CA | GLY | A | 243 | 43.011 | 62.995 | 157.379 | 1.00 | 0.00 | xxxx | 1875 |
| ATOM | 1876 | C | GLY | A | 243 | 42.969 | 61.786 | 158.305 | 1.00 | 0.00 | xxxx | 1876 |
| ATOM | 1877 | O | GLY | A | 243 | 42.437 | 60.724 | 157.961 | 1.00 | 0.00 | xxxx | 1877 |
| ATOM | 1878 | N | ILE | A | 244 | 43.533 | 61.955 | 159.499 | 1.00 | 0.00 | xxxx | 1878 |
| ATOM | 1879 | CA | ILE | A | 244 | 43.549 | 60.895 | 160.500 | 1.00 | 0.00 | xxxx | 1879 |
| ATOM | 1880 | CB | ILE | A | 244 | 44.333 | 61.342 | 161.748 | 1.00 | 0.00 | xxxx | 1880 |
| ATOM | 1881 | CG1 | ILE | A | 244 | 45.826 | 61.443 | 161.425 | 1.00 | 0.00 | xxxx | 1881 |
| ATOM | 1882 | CD1 | ILE | A | 244 | 46.597 | 62.226 | 162.456 | 1.00 | 0.00 | xxxx | 1882 |
| ATOM | 1883 | CG2 | ILE | A | 244 | 44.076 | 60.408 | 162.929 | 1.00 | 0.00 | xxxx | 1883 |
| ATOM | 1884 | C | ILE | A | 244 | 42.120 | 60.491 | 160.841 | 1.00 | 0.00 | xxxx | 1884 |
| ATOM | 1885 | O | ILE | A | 244 | 41.808 | 59.306 | 160.941 | 1.00 | 0.00 | xxxx | 1885 |
| ATOM | 1886 | N | GLN | A | 245 | 41.241 | 61.475 | 160.982 | 1.00 | 0.00 | xxxx | 1886 |
| ATOM | 1887 | CA | GLN | A | 245 | 39.851 | 61.177 | 161.287 | 1.00 | 0.00 | xxxx | 1887 |
| ATOM | 1888 | CB | GLN | A | 245 | 39.063 | 62.469 | 161.501 | 1.00 | 0.00 | xxxx | 1888 |
| ATOM | 1889 | CG | GLN | A | 245 | 37.649 | 62.224 | 161.982 | 1.00 | 0.00 | xxxx | 1889 |
| ATOM | 1890 | CD | GLN | A | 245 | 37.621 | 61.498 | 163.314 | 1.00 | 0.00 | xxxx | 1890 |
| ATOM | 1891 | OE1 | GLN | A | 245 | 38.162 | 61.987 | 164.309 | 1.00 | 0.00 | xxxx | 1891 |
| ATOM | 1892 | NE2 | GLN | A | 245 | 37.011 | 60.315 | 163.335 | 1.00 | 0.00 | xxxx | 1892 |
| ATOM | 1893 | C | GLN | A | 245 | 39.192 | 60.328 | 160.189 | 1.00 | 0.00 | xxxx | 1893 |
| ATOM | 1894 | O | GLN | A | 245 | 38.414 | 59.418 | 160.494 | 1.00 | 0.00 | xxxx | 1894 |
| ATOM | 1895 | N | ALA | A | 246 | 39.521 | 60.598 | 158.924 | 1.00 | 0.00 | xxxx | 1895 |
| ATOM | 1896 | CA | ALA | A | 246 | 38.941 | 59.829 | 157.819 | 1.00 | 0.00 | xxxx | 1896 |
| ATOM | 1897 | CB | ALA | A | 246 | 39.202 | 60.523 | 156.490 | 1.00 | 0.00 | xxxx | 1897 |
| ATOM | 1898 | C | ALA | A | 246 | 39.478 | 58.396 | 157.783 | 1.00 | 0.00 | xxxx | 1898 |
| ATOM | 1899 | O | ALA | A | 246 | 38.764 | 57.472 | 157.390 | 1.00 | 0.00 | xxxx | 1899 |
| ATOM | 1900 | N | ILE | A | 247 | 40.734 | 58.206 | 158.193 | 1.00 | 0.00 | xxxx | 1900 |
| ATOM | 1901 | CA | ILE | A | 247 | 41.272 | 56.854 | 158.343 | 1.00 | 0.00 | xxxx | 1901 |
| ATOM | 1902 | CB | ILE | A | 247 | 42.765 | 56.866 | 158.712 | 1.00 | 0.00 | xxxx | 1902 |
| ATOM | 1903 | CG1 | ILE | A | 247 | 43.583 | 57.467 | 157.566 | 1.00 | 0.00 | xxxx | 1903 |
| ATOM | 1904 | CD1 | ILE | A | 247 | 45.054 | 57.286 | 157.705 | 1.00 | 0.00 | xxxx | 1904 |
| ATOM | 1905 | CG2 | ILE | A | 247 | 43.249 | 55.456 | 159.063 | 1.00 | 0.00 | xxxx | 1905 |
| ATOM | 1906 | C | ILE | A | 247 | 40.469 | 56.101 | 159.392 | 1.00 | 0.00 | xxxx | 1906 |
| ATOM | 1907 | O | ILE | A | 247 | 40.092 | 54.951 | 159.199 | 1.00 | 0.00 | xxxx | 1907 |
| ATOM | 1908 | N | LYS | A | 248 | 40.209 | 56.767 | 160.509 | 1.00 | 0.00 | xxxx | 1908 |
| ATOM | 1909 | CA | LYS | A | 248 | 39.428 | 56.166 | 161.578 | 1.00 | 0.00 | xxxx | 1909 |
| ATOM | 1910 | CB | LYS | A | 248 | 39.441 | 57.071 | 162.803 | 1.00 | 0.00 | xxxx | 1910 |
| ATOM | 1911 | CG | LYS | A | 248 | 40.813 | 57.187 | 163.463 | 1.00 | 0.00 | xxxx | 1911 |
| ATOM | 1912 | CD | LYS | A | 248 | 40.756 | 58.156 | 164.623 | 1.00 | 0.00 | xxxx | 1912 |
| ATOM | 1913 | CE | LYS | A | 248 | 42.102 | 58.310 | 165.291 | 1.00 | 0.00 | xxxx | 1913 |
| ATOM | 1914 | NZ | LYS | A | 248 | 42.032 | 59.317 | 166.388 | 1.00 | 0.00 | xxxx | 1914 |
| ATOM | 1915 | C | LYS | A | 248 | 37.988 | 55.882 | 161.142 | 1.00 | 0.00 | xxxx | 1915 |
| ATOM | 1916 | O | LYS | A | 248 | 37.416 | 54.866 | 161.539 | 1.00 | 0.00 | xxxx | 1916 |
| ATOM | 1917 | N | ASP | A | 249 | 37.424 | 56.776 | 160.329 | 1.00 | 0.00 | xxxx | 1917 |
| ATOM | 1918 | CA | ASP | A | 249 | 36.057 | 56.640 | 159.803 | 1.00 | 0.00 | xxxx | 1918 |
| ATOM | 1919 | CB | ASP | A | 249 | 35.598 | 57.937 | 159.114 | 1.00 | 0.00 | xxxx | 1919 |
| ATOM | 1920 | CG | ASP | A | 249 | 35.429 | 59.101 | 160.078 | 1.00 | 0.00 | xxxx | 1920 |
| ATOM | 1921 | OD1 | ASP | A | 249 | 35.413 | 60.260 | 159.603 | 1.00 | 0.00 | xxxx | 1921 |
| ATOM | 1922 | OD2 | ASP | A | 249 | 35.308 | 58.871 | 161.301 | 1.00 | 0.00 | xxxx | 1922 |
| ATOM | 1923 | C | ASP | A | 249 | 35.949 | 55.489 | 158.798 | 1.00 | 0.00 | xxxx | 1923 |
| ATOM | 1924 | O | ASP | A | 249 | 34.853 | 55.002 | 158.507 | 1.00 | 0.00 | xxxx | 1924 |
| ATOM | 1925 | N | GLY | A | 250 | 37.091 | 55.085 | 158.251 | 1.00 | 0.00 | xxxx | 1925 |
| ATOM | 1926 | CA | GLY | A | 250 | 37.138 | 54.047 | 157.237 | 1.00 | 0.00 | xxxx | 1926 |
| ATOM | 1927 | C | GLY | A | 250 | 36.984 | 54.551 | 155.809 | 1.00 | 0.00 | xxxx | 1927 |
| ATOM | 1928 | O | GLY | A | 250 | 36.947 | 53.755 | 154.876 | 1.00 | 0.00 | xxxx | 1928 |
| ATOM | 1929 | N | THR | A | 251 | 36.900 | 55.868 | 155.636 | 1.00 | 0.00 | xxxx | 1929 |
| ATOM | 1930 | CA | THR | A | 251 | 36.671 | 56.458 | 154.313 | 1.00 | 0.00 | xxxx | 1930 |
| ATOM | 1931 | CB | THR | A | 251 | 35.837 | 57.756 | 154.403 | 1.00 | 0.00 | xxxx | 1931 |
| ATOM | 1932 | OG1 | THR | A | 251 | 36.531 | 58.731 | 155.198 | 1.00 | 0.00 | xxxx | 1932 |
| ATOM | 1933 | CG2 | THR | A | 251 | 34.449 | 57.472 | 154.995 | 1.00 | 0.00 | xxxx | 1933 |
| ATOM | 1934 | C | THR | A | 251 | 37.972 | 56.777 | 153.566 | 1.00 | 0.00 | xxxx | 1934 |
| ATOM | 1935 | O | THR | A | 251 | 37.973 | 56.923 | 152.346 | 1.00 | 0.00 | xxxx | 1935 |
| ATOM | 1936 | N | LEU | A | 252 | 39.070 | 56.900 | 154.306 | 1.00 | 0.00 | xxxx | 1936 |
| ATOM | 1937 | CA | LEU | A | 252 | 40.393 | 57.080 | 153.715 | 1.00 | 0.00 | xxxx | 1937 |
| ATOM | 1938 | CB | LEU | A | 252 | 41.089 | 58.317 | 154.297 | 1.00 | 0.00 | xxxx | 1938 |
| ATOM | 1939 | CG | LEU | A | 252 | 42.427 | 58.752 | 153.692 | 1.00 | 0.00 | xxxx | 1939 |
| ATOM | 1940 | CD1 | LEU | A | 252 | 42.273 | 59.128 | 152.211 | 1.00 | 0.00 | xxxx | 1940 |
| ATOM | 1941 | CD2 | LEU | A | 252 | 43.008 | 59.929 | 154.472 | 1.00 | 0.00 | xxxx | 1941 |
| ATOM | 1942 | C | LEU | A | 252 | 41.195 | 55.812 | 153.989 | 1.00 | 0.00 | xxxx | 1942 |
| ATOM | 1943 | O | LEU | A | 252 | 41.347 | 55.412 | 155.139 | 1.00 | 0.00 | xxxx | 1943 |
| ATOM | 1944 | N | LEU | A | 253 | 41.685 | 55.169 | 152.934 | 1.00 | 0.00 | xxxx | 1944 |
| ATOM | 1945 | CA | LEU | A | 253 | 42.397 | 53.903 | 153.086 | 1.00 | 0.00 | xxxx | 1945 |
| ATOM | 1946 | CB | LEU | A | 253 | 42.601 | 53.240 | 151.717 | 1.00 | 0.00 | xxxx | 1946 |
| ATOM | 1947 | CG | LEU | A | 253 | 43.518 | 52.011 | 151.707 | 1.00 | 0.00 | xxxx | 1947 |
| ATOM | 1948 | CD1 | LEU | A | 253 | 42.917 | 50.848 | 152.514 | 1.00 | 0.00 | xxxx | 1948 |
| ATOM | 1949 | CD2 | LEU | A | 253 | 43.811 | 51.593 | 150.278 | 1.00 | 0.00 | xxxx | 1949 |
| ATOM | 1950 | C | LEU | A | 253 | 43.742 | 54.105 | 153.776 | 1.00 | 0.00 | xxxx | 1950 |
| ATOM | 1951 | O | LEU | A | 253 | 44.149 | 53.333 | 154.648 | 1.00 | 0.00 | xxxx | 1951 |
| ATOM | 1952 | N | GLY | A | 254 | 44.441 | 55.157 | 153.389 | 1.00 | 0.00 | xxxx | 1952 |
| ATOM | 1953 | CA | GLY | A | 254 | 45.749 | 55.381 | 153.958 | 1.00 | 0.00 | xxxx | 1953 |
| ATOM | 1954 | C | GLY | A | 254 | 46.259 | 56.739 | 153.579 | 1.00 | 0.00 | xxxx | 1954 |
| ATOM | 1955 | O | GLY | A | 254 | 45.695 | 57.409 | 152.710 | 1.00 | 0.00 | xxxx | 1955 |
| ATOM | 1956 | N | THR | A | 255 | 47.328 | 57.144 | 154.249 | 1.00 | 0.00 | xxxx | 1956 |
| ATOM | 1957 | CA | THR | A | 255 | 47.980 | 58.395 | 153.941 | 1.00 | 0.00 | xxxx | 1957 |
| ATOM | 1958 | CB | THR | A | 255 | 47.293 | 59.602 | 154.650 | 1.00 | 0.00 | xxxx | 1958 |
| ATOM | 1959 | OG1 | THR | A | 255 | 47.875 | 60.829 | 154.206 | 1.00 | 0.00 | xxxx | 1959 |
| ATOM | 1960 | CG2 | THR | A | 255 | 47.392 | 59.517 | 156.170 | 1.00 | 0.00 | xxxx | 1960 |
| ATOM | 1961 | C | THR | A | 255 | 49.445 | 58.255 | 154.340 | 1.00 | 0.00 | xxxx | 1961 |
| ATOM | 1962 | O | THR | A | 255 | 49.913 | 57.156 | 154.636 | 1.00 | 0.00 | xxxx | 1962 |
| ATOM | 1963 | N | VAL | A | 256 | 50.169 | 59.363 | 154.283 | 1.00 | 0.00 | xxxx | 1963 |
| ATOM | 1964 | CA | VAL | A | 256 | 51.593 | 59.376 | 154.584 | 1.00 | 0.00 | xxxx | 1964 |
| ATOM | 1965 | CB | VAL | A | 256 | 52.434 | 59.484 | 153.297 | 1.00 | 0.00 | xxxx | 1965 |
| ATOM | 1966 | CG1 | VAL | A | 256 | 53.924 | 59.449 | 153.622 | 1.00 | 0.00 | xxxx | 1966 |
| ATOM | 1967 | CG2 | VAL | A | 256 | 52.054 | 58.386 | 152.302 | 1.00 | 0.00 | xxxx | 1967 |
| ATOM | 1968 | C | VAL | A | 256 | 51.827 | 60.559 | 155.495 | 1.00 | 0.00 | xxxx | 1968 |
| ATOM | 1969 | O | VAL | A | 256 | 51.471 | 61.677 | 155.149 | 1.00 | 0.00 | xxxx | 1969 |
| ATOM | 1970 | N | LEU | A | 257 | 52.399 | 60.316 | 156.668 | 1.00 | 0.00 | xxxx | 1970 |
| ATOM | 1971 | CA | LEU | A | 257 | 52.679 | 61.405 | 157.585 | 1.00 | 0.00 | xxxx | 1971 |
| ATOM | 1972 | CB | LEU | A | 257 | 53.143 | 60.875 | 158.938 | 1.00 | 0.00 | xxxx | 1972 |
| ATOM | 1973 | CG | LEU | A | 257 | 53.668 | 61.969 | 159.872 | 1.00 | 0.00 | xxxx | 1973 |
| ATOM | 1974 | CD1 | LEU | A | 257 | 52.555 | 62.938 | 160.270 | 1.00 | 0.00 | xxxx | 1974 |
| ATOM | 1975 | CD2 | LEU | A | 257 | 54.331 | 61.346 | 161.102 | 1.00 | 0.00 | xxxx | 1975 |
| ATOM | 1976 | C | LEU | A | 257 | 53.734 | 62.335 | 157.011 | 1.00 | 0.00 | xxxx | 1976 |
| ATOM | 1977 | O | LEU | A | 257 | 54.852 | 61.915 | 156.704 | 1.00 | 0.00 | xxxx | 1977 |
| ATOM | 1978 | N | ASN | A | 258 | 53.348 | 63.593 | 156.841 | 1.00 | 0.00 | xxxx | 1978 |
| ATOM | 1979 | CA | ASN | A | 258 | 54.252 | 64.663 | 156.459 | 1.00 | 0.00 | xxxx | 1979 |
| ATOM | 1980 | CB | ASN | A | 258 | 53.592 | 65.620 | 155.458 | 1.00 | 0.00 | xxxx | 1980 |
| ATOM | 1981 | CG | ASN | A | 258 | 54.584 | 66.574 | 154.797 | 1.00 | 0.00 | xxxx | 1981 |
| ATOM | 1982 | OD1 | ASN | A | 258 | 55.805 | 66.410 | 154.900 | 1.00 | 0.00 | xxxx | 1982 |
| ATOM | 1983 | ND2 | ASN | A | 258 | 54.054 | 67.560 | 154.084 | 1.00 | 0.00 | xxxx | 1983 |
| ATOM | 1984 | C | ASN | A | 258 | 54.577 | 65.344 | 157.771 | 1.00 | 0.00 | xxxx | 1984 |
| ATOM | 1985 | O | ASN | A | 258 | 53.705 | 65.942 | 158.400 | 1.00 | 0.00 | xxxx | 1985 |
| ATOM | 1986 | N | ASP | A | 259 | 55.822 | 65.207 | 158.207 | 1.00 | 0.00 | xxxx | 1986 |
| ATOM | 1987 | CA | ASP | A | 259 | 56.197 | 65.545 | 159.576 | 1.00 | 0.00 | xxxx | 1987 |
| ATOM | 1988 | CB | ASP | A | 259 | 57.455 | 64.762 | 159.955 | 1.00 | 0.00 | xxxx | 1988 |
| ATOM | 1989 | CG | ASP | A | 259 | 57.789 | 64.844 | 161.431 | 1.00 | 0.00 | xxxx | 1989 |
| ATOM | 1990 | OD1 | ASP | A | 259 | 57.216 | 65.690 | 162.151 | 1.00 | 0.00 | xxxx | 1990 |
| ATOM | 1991 | OD2 | ASP | A | 259 | 58.660 | 64.056 | 161.860 | 1.00 | 0.00 | xxxx | 1991 |
| ATOM | 1992 | C | ASP | A | 259 | 56.415 | 67.048 | 159.739 | 1.00 | 0.00 | xxxx | 1992 |
| ATOM | 1993 | O | ASP | A | 259 | 57.549 | 67.531 | 159.710 | 1.00 | 0.00 | xxxx | 1993 |
| ATOM | 1994 | N | ALA | A | 260 | 55.314 | 67.772 | 159.923 | 1.00 | 0.00 | xxxx | 1994 |
| ATOM | 1995 | CA | ALA | A | 260 | 55.334 | 69.218 | 160.026 | 1.00 | 0.00 | xxxx | 1995 |
| ATOM | 1996 | CB | ALA | A | 260 | 53.903 | 69.762 | 160.105 | 1.00 | 0.00 | xxxx | 1996 |
| ATOM | 1997 | C | ALA | A | 260 | 56.135 | 69.680 | 161.231 | 1.00 | 0.00 | xxxx | 1997 |
| ATOM | 1998 | O | ALA | A | 260 | 56.814 | 70.704 | 161.175 | 1.00 | 0.00 | xxxx | 1998 |
| ATOM | 1999 | N | LYS | A | 261 | 56.045 | 68.940 | 162.330 | 1.00 | 0.00 | xxxx | 1999 |
| ATOM | 2000 | CA | LYS | A | 261 | 56.710 | 69.369 | 163.549 | 1.00 | 0.00 | xxxx | 2000 |
| ATOM | 2001 | CB | LYS | A | 261 | 56.311 | 68.484 | 164.731 | 1.00 | 0.00 | xxxx | 2001 |
| ATOM | 2002 | CG | LYS | A | 261 | 54.865 | 68.702 | 165.160 | 1.00 | 0.00 | xxxx | 2002 |
| ATOM | 2003 | CD | LYS | A | 261 | 54.537 | 68.016 | 166.478 | 1.00 | 0.00 | xxxx | 2003 |
| ATOM | 2004 | CE | LYS | A | 261 | 53.071 | 68.221 | 166.846 | 1.00 | 0.00 | xxxx | 2004 |
| ATOM | 2005 | NZ | LYS | A | 261 | 52.725 | 67.634 | 168.170 | 1.00 | 0.00 | xxxx | 2005 |
| ATOM | 2006 | C | LYS | A | 261 | 58.224 | 69.387 | 163.372 | 1.00 | 0.00 | xxxx | 2006 |
| ATOM | 2007 | O | LYS | A | 261 | 58.870 | 70.364 | 163.743 | 1.00 | 0.00 | xxxx | 2007 |
| ATOM | 2008 | N | ASN | A | 262 | 58.800 | 68.333 | 162.797 | 1.00 | 0.00 | xxxx | 2008 |
| ATOM | 2009 | CA | ASN | A | 262 | 60.248 | 68.328 | 162.610 | 1.00 | 0.00 | xxxx | 2009 |
| ATOM | 2010 | CB | ASN | A | 262 | 60.767 | 66.905 | 162.398 | 1.00 | 0.00 | xxxx | 2010 |
| ATOM | 2011 | CG | ASN | A | 262 | 60.950 | 66.161 | 163.715 | 1.00 | 0.00 | xxxx | 2011 |
| ATOM | 2012 | OD1 | ASN | A | 262 | 61.734 | 66.572 | 164.571 | 1.00 | 0.00 | xxxx | 2012 |
| ATOM | 2013 | ND2 | ASN | A | 262 | 60.231 | 65.060 | 163.878 | 1.00 | 0.00 | xxxx | 2013 |
| ATOM | 2014 | C | ASN | A | 262 | 60.688 | 69.230 | 161.460 | 1.00 | 0.00 | xxxx | 2014 |
| ATOM | 2015 | O | ASN | A | 262 | 61.773 | 69.810 | 161.513 | 1.00 | 0.00 | xxxx | 2015 |
| ATOM | 2016 | N | GLN | A | 263 | 59.846 | 69.388 | 160.444 | 1.00 | 0.00 | xxxx | 2016 |
| ATOM | 2017 | CA | GLN | A | 263 | 60.182 | 70.338 | 159.392 | 1.00 | 0.00 | xxxx | 2017 |
| ATOM | 2018 | CB | GLN | A | 263 | 59.218 | 70.222 | 158.212 | 1.00 | 0.00 | xxxx | 2018 |
| ATOM | 2019 | CG | GLN | A | 263 | 59.489 | 68.968 | 157.391 | 1.00 | 0.00 | xxxx | 2019 |
| ATOM | 2020 | CD | GLN | A | 263 | 58.548 | 68.809 | 156.221 | 1.00 | 0.00 | xxxx | 2020 |
| ATOM | 2021 | OE1 | GLN | A | 263 | 58.355 | 69.734 | 155.431 | 1.00 | 0.00 | xxxx | 2021 |
| ATOM | 2022 | NE2 | GLN | A | 263 | 57.948 | 67.632 | 156.107 | 1.00 | 0.00 | xxxx | 2022 |
| ATOM | 2023 | C | GLN | A | 263 | 60.197 | 71.754 | 159.974 | 1.00 | 0.00 | xxxx | 2023 |
| ATOM | 2024 | O | GLN | A | 263 | 61.091 | 72.548 | 159.664 | 1.00 | 0.00 | xxxx | 2024 |
| ATOM | 2025 | N | ALA | A | 264 | 59.242 | 72.058 | 160.852 | 1.00 | 0.00 | xxxx | 2025 |
| ATOM | 2026 | CA | ALA | A | 264 | 59.205 | 73.371 | 161.488 | 1.00 | 0.00 | xxxx | 2026 |
| ATOM | 2027 | CB | ALA | A | 264 | 57.929 | 73.540 | 162.298 | 1.00 | 0.00 | xxxx | 2027 |
| ATOM | 2028 | C | ALA | A | 264 | 60.423 | 73.593 | 162.385 | 1.00 | 0.00 | xxxx | 2028 |
| ATOM | 2029 | O | ALA | A | 264 | 61.001 | 74.682 | 162.396 | 1.00 | 0.00 | xxxx | 2029 |
| ATOM | 2030 | N | LYS | A | 265 | 60.797 | 72.576 | 163.158 | 1.00 | 0.00 | xxxx | 2030 |
| ATOM | 2031 | C | LYS | A | 265 | 63.235 | 72.909 | 163.247 | 1.00 | 0.00 | xxxx | 2031 |
| ATOM | 2032 | O | LYS | A | 265 | 64.056 | 73.762 | 163.597 | 1.00 | 0.00 | xxxx | 2032 |
| ATOM | 2033 | CA | LYS | A | 265 | 61.955 | 72.688 | 164.042 | 1.00 | 0.00 | xxxx | 2033 |
| ATOM | 2034 | CB | LYS | A | 265 | 62.109 | 71.443 | 164.921 | 1.00 | 0.00 | xxxx | 2034 |
| ATOM | 2035 | CG | LYS | A | 265 | 60.962 | 71.194 | 165.866 | 1.00 | 0.00 | xxxx | 2035 |
| ATOM | 2036 | CD | LYS | A | 265 | 61.328 | 70.146 | 166.902 | 1.00 | 0.00 | xxxx | 2036 |
| ATOM | 2037 | CE | LYS | A | 265 | 60.117 | 69.716 | 167.711 | 1.00 | 0.00 | xxxx | 2037 |
| ATOM | 2038 | NZ | LYS | A | 265 | 60.514 | 68.939 | 168.913 | 1.00 | 0.00 | xxxx | 2038 |
| ATOM | 2039 | N | ALA | A | 266 | 63.409 | 72.146 | 162.171 | 1.00 | 0.00 | xxxx | 2039 |
| ATOM | 2040 | CA | ALA | A | 266 | 64.607 | 72.292 | 161.350 | 1.00 | 0.00 | xxxx | 2040 |
| ATOM | 2041 | CB | ALA | A | 266 | 64.661 | 71.209 | 160.261 | 1.00 | 0.00 | xxxx | 2041 |
| ATOM | 2042 | C | ALA | A | 266 | 64.656 | 73.682 | 160.727 | 1.00 | 0.00 | xxxx | 2042 |
| ATOM | 2043 | O | ALA | A | 266 | 65.702 | 74.325 | 160.708 | 1.00 | 0.00 | xxxx | 2043 |
| ATOM | 2044 | N | THR | A | 267 | 63.519 | 74.139 | 160.214 | 1.00 | 0.00 | xxxx | 2044 |
| ATOM | 2045 | CA | THR | A | 267 | 63.451 | 75.457 | 159.590 | 1.00 | 0.00 | xxxx | 2045 |
| ATOM | 2046 | CB | THR | A | 267 | 62.071 | 75.717 | 158.972 | 1.00 | 0.00 | xxxx | 2046 |
| ATOM | 2047 | OG1 | THR | A | 267 | 61.793 | 74.702 | 158.001 | 1.00 | 0.00 | xxxx | 2047 |
| ATOM | 2048 | CG2 | THR | A | 267 | 62.050 | 77.087 | 158.298 | 1.00 | 0.00 | xxxx | 2048 |
| ATOM | 2049 | C | THR | A | 267 | 63.769 | 76.550 | 160.594 | 1.00 | 0.00 | xxxx | 2049 |
| ATOM | 2050 | O | THR | A | 267 | 64.613 | 77.413 | 160.341 | 1.00 | 0.00 | xxxx | 2050 |
| ATOM | 2051 | N | PHE | A | 268 | 63.097 | 76.514 | 161.739 | 1.00 | 0.00 | xxxx | 2051 |
| ATOM | 2052 | CA | PHE | A | 268 | 63.396 | 77.493 | 162.764 | 1.00 | 0.00 | xxxx | 2052 |
| ATOM | 2053 | CB | PHE | A | 268 | 62.517 | 77.332 | 164.001 | 1.00 | 0.00 | xxxx | 2053 |
| ATOM | 2054 | CG | PHE | A | 268 | 62.907 | 78.279 | 165.090 | 1.00 | 0.00 | xxxx | 2054 |
| ATOM | 2055 | CD1 | PHE | A | 268 | 62.579 | 79.619 | 164.989 | 1.00 | 0.00 | xxxx | 2055 |
| ATOM | 2056 | CE1 | PHE | A | 268 | 62.971 | 80.513 | 165.958 | 1.00 | 0.00 | xxxx | 2056 |
| ATOM | 2057 | CZ | PHE | A | 268 | 63.722 | 80.073 | 167.033 | 1.00 | 0.00 | xxxx | 2057 |
| ATOM | 2058 | CE2 | PHE | A | 268 | 64.076 | 78.746 | 167.131 | 1.00 | 0.00 | xxxx | 2058 |
| ATOM | 2059 | CD2 | PHE | A | 268 | 63.679 | 77.855 | 166.161 | 1.00 | 0.00 | xxxx | 2059 |
| ATOM | 2060 | C | PHE | A | 268 | 64.858 | 77.423 | 163.204 | 1.00 | 0.00 | xxxx | 2060 |
| ATOM | 2061 | O | PHE | A | 268 | 65.507 | 78.456 | 163.318 | 1.00 | 0.00 | xxxx | 2061 |
| ATOM | 2062 | N | ASN | A | 269 | 65.367 | 76.219 | 163.467 | 1.00 | 0.00 | xxxx | 2062 |
| ATOM | 2063 | CA | ASN | A | 269 | 66.732 | 76.097 | 163.977 | 1.00 | 0.00 | xxxx | 2063 |
| ATOM | 2064 | CB | ASN | A | 269 | 67.074 | 74.636 | 164.290 | 1.00 | 0.00 | xxxx | 2064 |
| ATOM | 2065 | CG | ASN | A | 269 | 66.398 | 74.148 | 165.556 | 1.00 | 0.00 | xxxx | 2065 |
| ATOM | 2066 | OD1 | ASN | A | 269 | 65.773 | 74.931 | 166.270 | 1.00 | 0.00 | xxxx | 2066 |
| ATOM | 2067 | ND2 | ASN | A | 269 | 66.531 | 72.858 | 165.852 | 1.00 | 0.00 | xxxx | 2067 |
| ATOM | 2068 | C | ASN | A | 269 | 67.744 | 76.682 | 162.996 | 1.00 | 0.00 | xxxx | 2068 |
| ATOM | 2069 | O | ASN | A | 269 | 68.693 | 77.350 | 163.408 | 1.00 | 0.00 | xxxx | 2069 |
| ATOM | 2070 | N | ILE | A | 270 | 67.527 | 76.457 | 161.705 | 1.00 | 0.00 | xxxx | 2070 |
| ATOM | 2071 | CA | ILE | A | 270 | 68.428 | 77.004 | 160.704 | 1.00 | 0.00 | xxxx | 2071 |
| ATOM | 2072 | CB | ILE | A | 270 | 68.120 | 76.434 | 159.308 | 1.00 | 0.00 | xxxx | 2072 |
| ATOM | 2073 | CG1 | ILE | A | 270 | 68.529 | 74.959 | 159.220 | 1.00 | 0.00 | xxxx | 2073 |
| ATOM | 2074 | CD1 | ILE | A | 270 | 68.081 | 74.296 | 157.903 | 1.00 | 0.00 | xxxx | 2074 |
| ATOM | 2075 | CG2 | ILE | A | 270 | 68.811 | 77.279 | 158.217 | 1.00 | 0.00 | xxxx | 2075 |
| ATOM | 2076 | C | ILE | A | 270 | 68.360 | 78.532 | 160.701 | 1.00 | 0.00 | xxxx | 2076 |
| ATOM | 2077 | O | ILE | A | 270 | 69.394 | 79.200 | 160.723 | 1.00 | 0.00 | xxxx | 2077 |
| ATOM | 2078 | N | ALA | A | 271 | 67.148 | 79.083 | 160.699 | 1.00 | 0.00 | xxxx | 2078 |
| ATOM | 2079 | CA | ALA | A | 271 | 66.969 | 80.538 | 160.675 | 1.00 | 0.00 | xxxx | 2079 |
| ATOM | 2080 | CB | ALA | A | 271 | 65.482 | 80.900 | 160.547 | 1.00 | 0.00 | xxxx | 2080 |
| ATOM | 2081 | C | ALA | A | 271 | 67.569 | 81.163 | 161.928 | 1.00 | 0.00 | xxxx | 2081 |
| ATOM | 2082 | O | ALA | A | 271 | 68.144 | 82.248 | 161.877 | 1.00 | 0.00 | xxxx | 2082 |
| ATOM | 2083 | N | TYR | A | 272 | 67.439 | 80.457 | 163.047 | 1.00 | 0.00 | xxxx | 2083 |
| ATOM | 2084 | CA | TYR | A | 272 | 67.902 | 80.941 | 164.340 | 1.00 | 0.00 | xxxx | 2084 |
| ATOM | 2085 | CB | TYR | A | 272 | 67.345 | 80.034 | 165.445 | 1.00 | 0.00 | xxxx | 2085 |
| ATOM | 2086 | CG | TYR | A | 272 | 67.624 | 80.441 | 166.876 | 1.00 | 0.00 | xxxx | 2086 |
| ATOM | 2087 | CD1 | TYR | A | 272 | 67.292 | 81.704 | 167.345 | 1.00 | 0.00 | xxxx | 2087 |
| ATOM | 2088 | CE1 | TYR | A | 272 | 67.527 | 82.060 | 168.663 | 1.00 | 0.00 | xxxx | 2088 |
| ATOM | 2089 | CZ | TYR | A | 272 | 68.082 | 81.142 | 169.528 | 1.00 | 0.00 | xxxx | 2089 |
| ATOM | 2090 | OH | TYR | A | 272 | 68.318 | 81.500 | 170.838 | 1.00 | 0.00 | xxxx | 2090 |
| ATOM | 2091 | CE2 | TYR | A | 272 | 68.407 | 79.877 | 169.092 | 1.00 | 0.00 | xxxx | 2091 |
| ATOM | 2092 | CD2 | TYR | A | 272 | 68.165 | 79.529 | 167.773 | 1.00 | 0.00 | xxxx | 2092 |
| ATOM | 2093 | C | TYR | A | 272 | 69.426 | 80.990 | 164.382 | 1.00 | 0.00 | xxxx | 2093 |
| ATOM | 2094 | O | TYR | A | 272 | 70.008 | 81.970 | 164.852 | 1.00 | 0.00 | xxxx | 2094 |
| ATOM | 2095 | N | GLU | A | 273 | 70.076 | 79.939 | 163.879 | 1.00 | 0.00 | xxxx | 2095 |
| ATOM | 2096 | CA | GLU | A | 273 | 71.533 | 79.939 | 163.820 | 1.00 | 0.00 | xxxx | 2096 |
| ATOM | 2097 | CB | GLU | A | 273 | 72.076 | 78.576 | 163.385 | 1.00 | 0.00 | xxxx | 2097 |
| ATOM | 2098 | CG | GLU | A | 273 | 71.808 | 77.455 | 164.387 | 1.00 | 0.00 | xxxx | 2098 |
| ATOM | 2099 | CD | GLU | A | 273 | 72.396 | 77.727 | 165.771 | 1.00 | 0.00 | xxxx | 2099 |
| ATOM | 2100 | OE1 | GLU | A | 273 | 73.574 | 78.132 | 165.864 | 1.00 | 0.00 | xxxx | 2100 |
| ATOM | 2101 | OE2 | GLU | A | 273 | 71.674 | 77.533 | 166.771 | 1.00 | 0.00 | xxxx | 2101 |
| ATOM | 2102 | C | GLU | A | 273 | 72.017 | 81.037 | 162.873 | 1.00 | 0.00 | xxxx | 2102 |
| ATOM | 2103 | O | GLU | A | 273 | 72.927 | 81.795 | 163.210 | 1.00 | 0.00 | xxxx | 2103 |
| ATOM | 2104 | N | LEU | A | 274 | 71.394 | 81.136 | 161.701 | 1.00 | 0.00 | xxxx | 2104 |
| ATOM | 2105 | CA | LEU | A | 274 | 71.817 | 82.128 | 160.715 | 1.00 | 0.00 | xxxx | 2105 |
| ATOM | 2106 | CB | LEU | A | 274 | 71.085 | 81.928 | 159.385 | 1.00 | 0.00 | xxxx | 2106 |
| ATOM | 2107 | CG | LEU | A | 274 | 71.476 | 80.692 | 158.562 | 1.00 | 0.00 | xxxx | 2107 |
| ATOM | 2108 | CD1 | LEU | A | 274 | 70.465 | 80.446 | 157.451 | 1.00 | 0.00 | xxxx | 2108 |
| ATOM | 2109 | CD2 | LEU | A | 274 | 72.894 | 80.826 | 158.000 | 1.00 | 0.00 | xxxx | 2109 |
| ATOM | 2110 | C | LEU | A | 274 | 71.592 | 83.545 | 161.240 | 1.00 | 0.00 | xxxx | 2110 |
| ATOM | 2111 | O | LEU | A | 274 | 72.384 | 84.450 | 160.968 | 1.00 | 0.00 | xxxx | 2111 |
| ATOM | 2112 | N | ALA | A | 275 | 70.533 | 83.727 | 162.023 | 1.00 | 0.00 | xxxx | 2112 |
| ATOM | 2113 | CA | ALA | A | 275 | 70.234 | 85.034 | 162.599 | 1.00 | 0.00 | xxxx | 2113 |
| ATOM | 2114 | CB | ALA | A | 275 | 68.877 | 85.021 | 163.283 | 1.00 | 0.00 | xxxx | 2114 |
| ATOM | 2115 | C | ALA | A | 275 | 71.323 | 85.449 | 163.585 | 1.00 | 0.00 | xxxx | 2115 |
| ATOM | 2116 | O | ALA | A | 275 | 71.541 | 86.641 | 163.813 | 1.00 | 0.00 | xxxx | 2116 |
| ATOM | 2117 | N | GLN | A | 276 | 71.997 | 84.458 | 164.165 | 1.00 | 0.00 | xxxx | 2117 |
| ATOM | 2118 | CA | GLN | A | 276 | 73.079 | 84.706 | 165.120 | 1.00 | 0.00 | xxxx | 2118 |
| ATOM | 2119 | CB | GLN | A | 276 | 73.124 | 83.613 | 166.189 | 1.00 | 0.00 | xxxx | 2119 |
| ATOM | 2120 | CG | GLN | A | 276 | 71.901 | 83.542 | 167.092 | 1.00 | 0.00 | xxxx | 2120 |
| ATOM | 2121 | CD | GLN | A | 276 | 71.914 | 82.318 | 167.986 | 1.00 | 0.00 | xxxx | 2121 |
| ATOM | 2122 | OE1 | GLN | A | 276 | 71.240 | 81.323 | 167.708 | 1.00 | 0.00 | xxxx | 2122 |
| ATOM | 2123 | NE2 | GLN | A | 276 | 72.690 | 82.381 | 169.066 | 1.00 | 0.00 | xxxx | 2123 |
| ATOM | 2124 | C | GLN | A | 276 | 74.442 | 84.790 | 164.440 | 1.00 | 0.00 | xxxx | 2124 |
| ATOM | 2125 | O | GLN | A | 276 | 75.461 | 84.971 | 165.110 | 1.00 | 0.00 | xxxx | 2125 |
| ATOM | 2126 | N | GLY | A | 277 | 74.461 | 84.662 | 163.119 | 1.00 | 0.00 | xxxx | 2126 |
| ATOM | 2127 | CA | GLY | A | 277 | 75.708 | 84.670 | 162.372 | 1.00 | 0.00 | xxxx | 2127 |
| ATOM | 2128 | C | GLY | A | 277 | 76.469 | 83.358 | 162.461 | 1.00 | 0.00 | xxxx | 2128 |
| ATOM | 2129 | O | GLY | A | 277 | 77.673 | 83.303 | 162.200 | 1.00 | 0.00 | xxxx | 2129 |
| ATOM | 2130 | N | ILE | A | 278 | 75.762 | 82.293 | 162.823 | 1.00 | 0.00 | xxxx | 2130 |
| ATOM | 2131 | CA | ILE | A | 278 | 76.368 | 80.971 | 162.968 | 1.00 | 0.00 | xxxx | 2131 |
| ATOM | 2132 | CB | ILE | A | 278 | 75.943 | 80.326 | 164.299 | 1.00 | 0.00 | xxxx | 2132 |
| ATOM | 2133 | CG1 | ILE | A | 278 | 76.389 | 81.209 | 165.470 | 1.00 | 0.00 | xxxx | 2133 |
| ATOM | 2134 | CD1 | ILE | A | 278 | 75.767 | 80.832 | 166.795 | 1.00 | 0.00 | xxxx | 2134 |
| ATOM | 2135 | CG2 | ILE | A | 278 | 76.495 | 78.904 | 164.424 | 1.00 | 0.00 | xxxx | 2135 |
| ATOM | 2136 | C | ILE | A | 278 | 75.998 | 80.078 | 161.782 | 1.00 | 0.00 | xxxx | 2136 |
| ATOM | 2137 | O | ILE | A | 278 | 74.836 | 80.015 | 161.380 | 1.00 | 0.00 | xxxx | 2137 |
| ATOM | 2138 | N | THR | A | 279 | 76.988 | 79.390 | 161.221 | 1.00 | 0.00 | xxxx | 2138 |
| ATOM | 2139 | CA | THR | A | 279 | 76.737 | 78.469 | 160.112 | 1.00 | 0.00 | xxxx | 2139 |
| ATOM | 2140 | CB | THR | A | 279 | 78.040 | 78.105 | 159.374 | 1.00 | 0.00 | xxxx | 2140 |
| ATOM | 2141 | OG1 | THR | A | 279 | 78.635 | 79.296 | 158.841 | 1.00 | 0.00 | xxxx | 2141 |
| ATOM | 2142 | CG2 | THR | A | 279 | 77.760 | 77.132 | 158.245 | 1.00 | 0.00 | xxxx | 2142 |
| ATOM | 2143 | C | THR | A | 279 | 76.078 | 77.186 | 160.609 | 1.00 | 0.00 | xxxx | 2143 |
| ATOM | 2144 | O | THR | A | 279 | 76.610 | 76.533 | 161.506 | 1.00 | 0.00 | xxxx | 2144 |
| ATOM | 2145 | N | PRO | A | 280 | 74.920 | 76.819 | 160.027 | 1.00 | 0.00 | xxxx | 2145 |
| ATOM | 2146 | CA | PRO | A | 280 | 74.256 | 75.568 | 160.417 | 1.00 | 0.00 | xxxx | 2146 |
| ATOM | 2147 | CB | PRO | A | 280 | 73.070 | 75.489 | 159.450 | 1.00 | 0.00 | xxxx | 2147 |
| ATOM | 2148 | CG | PRO | A | 280 | 72.751 | 76.924 | 159.144 | 1.00 | 0.00 | xxxx | 2148 |
| ATOM | 2149 | CD | PRO | A | 280 | 74.092 | 77.612 | 159.097 | 1.00 | 0.00 | xxxx | 2149 |
| ATOM | 2150 | C | PRO | A | 280 | 75.140 | 74.329 | 160.279 | 1.00 | 0.00 | xxxx | 2150 |
| ATOM | 2151 | O | PRO | A | 280 | 75.859 | 74.177 | 159.290 | 1.00 | 0.00 | xxxx | 2151 |
| ATOM | 2152 | N | THR | A | 281 | 75.102 | 73.472 | 161.295 | 1.00 | 0.00 | xxxx | 2152 |
| ATOM | 2153 | CA | THR | A | 281 | 75.739 | 72.155 | 161.265 | 1.00 | 0.00 | xxxx | 2153 |
| ATOM | 2154 | CB | THR | A | 281 | 77.009 | 72.086 | 162.122 | 1.00 | 0.00 | xxxx | 2154 |
| ATOM | 2155 | OG1 | THR | A | 281 | 76.650 | 72.211 | 163.503 | 1.00 | 0.00 | xxxx | 2155 |
| ATOM | 2156 | CG2 | THR | A | 281 | 77.978 | 73.199 | 161.759 | 1.00 | 0.00 | xxxx | 2156 |
| ATOM | 2157 | C | THR | A | 281 | 74.728 | 71.158 | 161.808 | 1.00 | 0.00 | xxxx | 2157 |
| ATOM | 2158 | O | THR | A | 281 | 73.770 | 71.560 | 162.466 | 1.00 | 0.00 | xxxx | 2158 |
| ATOM | 2159 | N | LYS | A | 282 | 74.939 | 69.868 | 161.558 | 1.00 | 0.00 | xxxx | 2159 |
| ATOM | 2160 | CA | LYS | A | 282 | 74.074 | 68.851 | 162.146 | 1.00 | 0.00 | xxxx | 2160 |
| ATOM | 2161 | CB | LYS | A | 282 | 74.540 | 67.441 | 161.777 | 1.00 | 0.00 | xxxx | 2161 |
| ATOM | 2162 | CG | LYS | A | 282 | 73.709 | 66.356 | 162.445 | 1.00 | 0.00 | xxxx | 2162 |
| ATOM | 2163 | CD | LYS | A | 282 | 74.069 | 64.971 | 161.951 | 1.00 | 0.00 | xxxx | 2163 |
| ATOM | 2164 | CE | LYS | A | 282 | 73.131 | 63.935 | 162.548 | 1.00 | 0.00 | xxxx | 2164 |
| ATOM | 2165 | NZ | LYS | A | 282 | 73.423 | 62.568 | 162.033 | 1.00 | 0.00 | xxxx | 2165 |
| ATOM | 2166 | C | LYS | A | 282 | 74.026 | 68.995 | 163.667 | 1.00 | 0.00 | xxxx | 2166 |
| ATOM | 2167 | O | LYS | A | 282 | 72.974 | 68.811 | 164.278 | 1.00 | 0.00 | xxxx | 2167 |
| ATOM | 2168 | N | ASP | A | 283 | 75.156 | 69.342 | 164.274 | 1.00 | 0.00 | xxxx | 2168 |
| ATOM | 2169 | CA | ASP | A | 283 | 75.208 | 69.504 | 165.719 | 1.00 | 0.00 | xxxx | 2169 |
| ATOM | 2170 | CB | ASP | A | 283 | 76.646 | 69.739 | 166.193 | 1.00 | 0.00 | xxxx | 2170 |
| ATOM | 2171 | CG | ASP | A | 283 | 77.478 | 68.474 | 166.201 | 1.00 | 0.00 | xxxx | 2171 |
| ATOM | 2172 | OD1 | ASP | A | 283 | 76.920 | 67.381 | 165.965 | 1.00 | 0.00 | xxxx | 2172 |
| ATOM | 2173 | OD2 | ASP | A | 283 | 78.698 | 68.576 | 166.461 | 1.00 | 0.00 | xxxx | 2173 |
| ATOM | 2174 | C | ASP | A | 283 | 74.329 | 70.651 | 166.217 | 1.00 | 0.00 | xxxx | 2174 |
| ATOM | 2175 | O | ASP | A | 283 | 73.639 | 70.501 | 167.228 | 1.00 | 0.00 | xxxx | 2175 |
| ATOM | 2176 | N | ASN | A | 284 | 74.353 | 71.799 | 165.539 | 1.00 | 0.00 | xxxx | 2176 |
| ATOM | 2177 | CA | ASN | A | 284 | 73.670 | 72.962 | 166.110 | 1.00 | 0.00 | xxxx | 2177 |
| ATOM | 2178 | CB | ASN | A | 284 | 74.506 | 74.254 | 165.920 | 1.00 | 0.00 | xxxx | 2178 |
| ATOM | 2179 | CG | ASN | A | 284 | 74.696 | 74.658 | 164.461 | 1.00 | 0.00 | xxxx | 2179 |
| ATOM | 2180 | OD1 | ASN | A | 284 | 73.869 | 74.373 | 163.603 | 1.00 | 0.00 | xxxx | 2180 |
| ATOM | 2181 | ND2 | ASN | A | 284 | 75.793 | 75.361 | 164.188 | 1.00 | 0.00 | xxxx | 2181 |
| ATOM | 2182 | C | ASN | A | 284 | 72.237 | 73.163 | 165.593 | 1.00 | 0.00 | xxxx | 2182 |
| ATOM | 2183 | O | ASN | A | 284 | 71.515 | 73.997 | 166.135 | 1.00 | 0.00 | xxxx | 2183 |
| ATOM | 2184 | N | ILE | A | 285 | 71.805 | 72.403 | 164.581 | 1.00 | 0.00 | xxxx | 2184 |
| ATOM | 2185 | CA | ILE | A | 285 | 70.383 | 72.428 | 164.228 | 1.00 | 0.00 | xxxx | 2185 |
| ATOM | 2186 | CB | ILE | A | 285 | 70.154 | 72.786 | 162.739 | 1.00 | 0.00 | xxxx | 2186 |
| ATOM | 2187 | CG1 | ILE | A | 285 | 70.630 | 71.667 | 161.808 | 1.00 | 0.00 | xxxx | 2187 |
| ATOM | 2188 | CD1 | ILE | A | 285 | 70.062 | 71.779 | 160.390 | 1.00 | 0.00 | xxxx | 2188 |
| ATOM | 2189 | CG2 | ILE | A | 285 | 70.809 | 74.122 | 162.399 | 1.00 | 0.00 | xxxx | 2189 |
| ATOM | 2190 | C | ILE | A | 285 | 69.662 | 71.116 | 164.557 | 1.00 | 0.00 | xxxx | 2190 |
| ATOM | 2191 | O | ILE | A | 285 | 68.431 | 71.087 | 164.592 | 1.00 | 0.00 | xxxx | 2191 |
| ATOM | 2192 | N | GLY | A | 286 | 70.414 | 70.040 | 164.782 | 1.00 | 0.00 | xxxx | 2192 |
| ATOM | 2193 | CA | GLY | A | 286 | 69.830 | 68.779 | 165.210 | 1.00 | 0.00 | xxxx | 2193 |
| ATOM | 2194 | C | GLY | A | 286 | 69.321 | 67.841 | 164.134 | 1.00 | 0.00 | xxxx | 2194 |
| ATOM | 2195 | O | GLY | A | 286 | 68.576 | 66.908 | 164.437 | 1.00 | 0.00 | xxxx | 2195 |
| ATOM | 2196 | N | TYR | A | 287 | 69.718 | 68.088 | 162.883 | 1.00 | 0.00 | xxxx | 2196 |
| ATOM | 2197 | CA | TYR | A | 287 | 69.293 | 67.281 | 161.736 | 1.00 | 0.00 | xxxx | 2197 |
| ATOM | 2198 | CB | TYR | A | 287 | 68.093 | 67.922 | 161.026 | 1.00 | 0.00 | xxxx | 2198 |
| ATOM | 2199 | CG | TYR | A | 287 | 66.884 | 68.058 | 161.911 | 1.00 | 0.00 | xxxx | 2199 |
| ATOM | 2200 | CD1 | TYR | A | 287 | 66.708 | 69.188 | 162.701 | 1.00 | 0.00 | xxxx | 2200 |
| ATOM | 2201 | CE1 | TYR | A | 287 | 65.613 | 69.318 | 163.527 | 1.00 | 0.00 | xxxx | 2201 |
| ATOM | 2202 | CZ | TYR | A | 287 | 64.674 | 68.312 | 163.570 | 1.00 | 0.00 | xxxx | 2202 |
| ATOM | 2203 | OH | TYR | A | 287 | 63.586 | 68.449 | 164.405 | 1.00 | 0.00 | xxxx | 2203 |
| ATOM | 2204 | CE2 | TYR | A | 287 | 64.823 | 67.171 | 162.795 | 1.00 | 0.00 | xxxx | 2204 |
| ATOM | 2205 | CD2 | TYR | A | 287 | 65.923 | 67.052 | 161.965 | 1.00 | 0.00 | xxxx | 2205 |
| ATOM | 2206 | C | TYR | A | 287 | 70.428 | 67.138 | 160.738 | 1.00 | 0.00 | xxxx | 2206 |
| ATOM | 2207 | O | TYR | A | 287 | 71.245 | 68.048 | 160.609 | 1.00 | 0.00 | xxxx | 2207 |
| ATOM | 2208 | N | ASP | A | 288 | 70.469 | 66.004 | 160.034 | 1.00 | 0.00 | xxxx | 2208 |
| ATOM | 2209 | CA | ASP | A | 288 | 71.399 | 65.819 | 158.918 | 1.00 | 0.00 | xxxx | 2209 |
| ATOM | 2210 | CB | ASP | A | 288 | 71.210 | 64.445 | 158.269 | 1.00 | 0.00 | xxxx | 2210 |
| ATOM | 2211 | CG | ASP | A | 288 | 71.732 | 63.318 | 159.118 | 1.00 | 0.00 | xxxx | 2211 |
| ATOM | 2212 | OD1 | ASP | A | 288 | 72.927 | 63.343 | 159.473 | 1.00 | 0.00 | xxxx | 2212 |
| ATOM | 2213 | OD2 | ASP | A | 288 | 70.942 | 62.397 | 159.416 | 1.00 | 0.00 | xxxx | 2213 |
| ATOM | 2214 | C | ASP | A | 288 | 71.203 | 66.877 | 157.843 | 1.00 | 0.00 | xxxx | 2214 |
| ATOM | 2215 | O | ASP | A | 288 | 70.071 | 67.186 | 157.466 | 1.00 | 0.00 | xxxx | 2215 |
| ATOM | 2216 | N | ILE | A | 289 | 72.311 | 67.412 | 157.340 | 1.00 | 0.00 | xxxx | 2216 |
| ATOM | 2217 | CA | ILE | A | 289 | 72.280 | 68.351 | 156.219 | 1.00 | 0.00 | xxxx | 2217 |
| ATOM | 2218 | CB | ILE | A | 289 | 73.095 | 69.615 | 156.517 | 1.00 | 0.00 | xxxx | 2218 |
| ATOM | 2219 | CG1 | ILE | A | 289 | 72.558 | 70.310 | 157.769 | 1.00 | 0.00 | xxxx | 2219 |
| ATOM | 2220 | CD1 | ILE | A | 289 | 73.458 | 71.434 | 158.271 | 1.00 | 0.00 | xxxx | 2220 |
| ATOM | 2221 | CG2 | ILE | A | 289 | 73.059 | 70.575 | 155.321 | 1.00 | 0.00 | xxxx | 2221 |
| ATOM | 2222 | C | ILE | A | 289 | 72.810 | 67.618 | 154.995 | 1.00 | 0.00 | xxxx | 2222 |
| ATOM | 2223 | O | ILE | A | 289 | 73.862 | 66.978 | 155.052 | 1.00 | 0.00 | xxxx | 2223 |
| ATOM | 2224 | N | THR | A | 290 | 72.066 | 67.701 | 153.898 | 1.00 | 0.00 | xxxx | 2224 |
| ATOM | 2225 | CA | THR | A | 290 | 72.393 | 66.983 | 152.668 | 1.00 | 0.00 | xxxx | 2225 |
| ATOM | 2226 | CB | THR | A | 290 | 71.173 | 66.165 | 152.178 | 1.00 | 0.00 | xxxx | 2226 |
| ATOM | 2227 | OG1 | THR | A | 290 | 70.769 | 65.253 | 153.204 | 1.00 | 0.00 | xxxx | 2227 |
| ATOM | 2228 | CG2 | THR | A | 290 | 71.496 | 65.377 | 150.909 | 1.00 | 0.00 | xxxx | 2228 |
| ATOM | 2229 | C | THR | A | 290 | 72.829 | 67.970 | 151.588 | 1.00 | 0.00 | xxxx | 2229 |
| ATOM | 2230 | O | THR | A | 290 | 72.252 | 69.046 | 151.462 | 1.00 | 0.00 | xxxx | 2230 |
| ATOM | 2231 | N | ASP | A | 291 | 73.872 | 67.620 | 150.841 | 1.00 | 0.00 | xxxx | 2231 |
| ATOM | 2232 | CA | ASP | A | 291 | 74.367 | 68.479 | 149.764 | 1.00 | 0.00 | xxxx | 2232 |
| ATOM | 2233 | CB | ASP | A | 291 | 73.338 | 68.541 | 148.614 | 1.00 | 0.00 | xxxx | 2233 |
| ATOM | 2234 | CG | ASP | A | 291 | 73.206 | 67.219 | 147.865 | 1.00 | 0.00 | xxxx | 2234 |
| ATOM | 2235 | OD1 | ASP | A | 291 | 74.245 | 66.647 | 147.474 | 1.00 | 0.00 | xxxx | 2235 |
| ATOM | 2236 | OD2 | ASP | A | 291 | 72.065 | 66.754 | 147.659 | 1.00 | 0.00 | xxxx | 2236 |
| ATOM | 2237 | C | ASP | A | 291 | 74.696 | 69.891 | 150.274 | 1.00 | 0.00 | xxxx | 2237 |
| ATOM | 2238 | O | ASP | A | 291 | 74.624 | 70.873 | 149.532 | 1.00 | 0.00 | xxxx | 2238 |
| ATOM | 2239 | N | GLY | A | 292 | 75.068 | 69.984 | 151.546 | 1.00 | 0.00 | xxxx | 2239 |
| ATOM | 2240 | CA | GLY | A | 292 | 75.441 | 71.251 | 152.152 | 1.00 | 0.00 | xxxx | 2240 |
| ATOM | 2241 | C | GLY | A | 292 | 74.328 | 72.257 | 152.390 | 1.00 | 0.00 | xxxx | 2241 |
| ATOM | 2242 | O | GLY | A | 292 | 74.558 | 73.299 | 153.006 | 1.00 | 0.00 | xxxx | 2242 |
| ATOM | 2243 | N | LYS | A | 293 | 73.118 | 71.958 | 151.920 | 1.00 | 0.00 | xxxx | 2243 |
| ATOM | 2244 | CA | LYS | A | 293 | 72.054 | 72.963 | 151.908 | 1.00 | 0.00 | xxxx | 2244 |
| ATOM | 2245 | CB | LYS | A | 293 | 71.920 | 73.565 | 150.505 | 1.00 | 0.00 | xxxx | 2245 |
| ATOM | 2246 | CG | LYS | A | 293 | 73.118 | 74.371 | 150.044 | 1.00 | 0.00 | xxxx | 2246 |
| ATOM | 2247 | CD | LYS | A | 293 | 72.954 | 74.836 | 148.612 | 1.00 | 0.00 | xxxx | 2247 |
| ATOM | 2248 | CE | LYS | A | 293 | 74.246 | 75.471 | 148.090 | 1.00 | 0.00 | xxxx | 2248 |
| ATOM | 2249 | NZ | LYS | A | 293 | 74.399 | 76.899 | 148.510 | 1.00 | 0.00 | xxxx | 2249 |
| ATOM | 2250 | C | LYS | A | 293 | 70.676 | 72.474 | 152.330 | 1.00 | 0.00 | xxxx | 2250 |
| ATOM | 2251 | O | LYS | A | 293 | 69.810 | 73.296 | 152.635 | 1.00 | 0.00 | xxxx | 2251 |
| ATOM | 2252 | N | TYR | A | 294 | 70.456 | 71.161 | 152.316 | 1.00 | 0.00 | xxxx | 2252 |
| ATOM | 2253 | CA | TYR | A | 294 | 69.104 | 70.620 | 152.456 | 1.00 | 0.00 | xxxx | 2253 |
| ATOM | 2254 | CB | TYR | A | 294 | 68.762 | 69.680 | 151.294 | 1.00 | 0.00 | xxxx | 2254 |
| ATOM | 2255 | CG | TYR | A | 294 | 68.805 | 70.293 | 149.917 | 1.00 | 0.00 | xxxx | 2255 |
| ATOM | 2256 | CD1 | TYR | A | 294 | 69.996 | 70.361 | 149.205 | 1.00 | 0.00 | xxxx | 2256 |
| ATOM | 2257 | CE1 | TYR | A | 294 | 70.032 | 70.915 | 147.941 | 1.00 | 0.00 | xxxx | 2257 |
| ATOM | 2258 | CZ | TYR | A | 294 | 68.863 | 71.389 | 147.366 | 1.00 | 0.00 | xxxx | 2258 |
| ATOM | 2259 | OH | TYR | A | 294 | 68.908 | 71.929 | 146.098 | 1.00 | 0.00 | xxxx | 2259 |
| ATOM | 2260 | CE2 | TYR | A | 294 | 67.672 | 71.319 | 148.046 | 1.00 | 0.00 | xxxx | 2260 |
| ATOM | 2261 | CD2 | TYR | A | 294 | 67.651 | 70.777 | 149.318 | 1.00 | 0.00 | xxxx | 2261 |
| ATOM | 2262 | C | TYR | A | 294 | 68.910 | 69.839 | 153.739 | 1.00 | 0.00 | xxxx | 2262 |
| ATOM | 2263 | O | TYR | A | 294 | 69.782 | 69.065 | 154.135 | 1.00 | 0.00 | xxxx | 2263 |
| ATOM | 2264 | N | VAL | A | 295 | 67.751 | 70.013 | 154.365 | 1.00 | 0.00 | xxxx | 2264 |
| ATOM | 2265 | CA | VAL | A | 295 | 67.357 | 69.122 | 155.445 | 1.00 | 0.00 | xxxx | 2265 |
| ATOM | 2266 | CB | VAL | A | 295 | 67.101 | 69.874 | 156.749 | 1.00 | 0.00 | xxxx | 2266 |
| ATOM | 2267 | CG1 | VAL | A | 295 | 66.510 | 68.914 | 157.789 | 1.00 | 0.00 | xxxx | 2267 |
| ATOM | 2268 | CG2 | VAL | A | 295 | 68.402 | 70.504 | 157.263 | 1.00 | 0.00 | xxxx | 2268 |
| ATOM | 2269 | C | VAL | A | 295 | 66.116 | 68.350 | 155.008 | 1.00 | 0.00 | xxxx | 2269 |
| ATOM | 2270 | O | VAL | A | 295 | 65.059 | 68.939 | 154.769 | 1.00 | 0.00 | xxxx | 2270 |
| ATOM | 2271 | N | TRP | A | 296 | 66.274 | 67.037 | 154.876 | 1.00 | 0.00 | xxxx | 2271 |
| ATOM | 2272 | CA | TRP | A | 296 | 65.187 | 66.147 | 154.483 | 1.00 | 0.00 | xxxx | 2272 |
| ATOM | 2273 | CB | TRP | A | 296 | 65.668 | 65.135 | 153.441 | 1.00 | 0.00 | xxxx | 2273 |
| ATOM | 2274 | CG | TRP | A | 296 | 66.233 | 65.760 | 152.203 | 1.00 | 0.00 | xxxx | 2274 |
| ATOM | 2275 | CD1 | TRP | A | 296 | 67.529 | 65.712 | 151.778 | 1.00 | 0.00 | xxxx | 2275 |
| ATOM | 2276 | NE1 | TRP | A | 296 | 67.665 | 66.395 | 150.595 | 1.00 | 0.00 | xxxx | 2276 |
| ATOM | 2277 | CE2 | TRP | A | 296 | 66.449 | 66.922 | 150.244 | 1.00 | 0.00 | xxxx | 2277 |
| ATOM | 2278 | CD2 | TRP | A | 296 | 65.522 | 66.542 | 151.235 | 1.00 | 0.00 | xxxx | 2278 |
| ATOM | 2279 | CE3 | TRP | A | 296 | 64.192 | 66.955 | 151.110 | 1.00 | 0.00 | xxxx | 2279 |
| ATOM | 2280 | CZ3 | TRP | A | 296 | 63.831 | 67.718 | 150.011 | 1.00 | 0.00 | xxxx | 2280 |
| ATOM | 2281 | CH2 | TRP | A | 296 | 64.776 | 68.082 | 149.041 | 1.00 | 0.00 | xxxx | 2281 |
| ATOM | 2282 | CZ2 | TRP | A | 296 | 66.091 | 67.694 | 149.141 | 1.00 | 0.00 | xxxx | 2282 |
| ATOM | 2283 | C | TRP | A | 296 | 64.626 | 65.422 | 155.691 | 1.00 | 0.00 | xxxx | 2283 |
| ATOM | 2284 | O | TRP | A | 296 | 65.356 | 64.767 | 156.437 | 1.00 | 0.00 | xxxx | 2284 |
| ATOM | 2285 | N | ILE | A | 297 | 63.319 | 65.556 | 155.886 | 1.00 | 0.00 | xxxx | 2285 |
| ATOM | 2286 | CA | ILE | A | 297 | 62.625 | 64.918 | 156.991 | 1.00 | 0.00 | xxxx | 2286 |
| ATOM | 2287 | CB | ILE | A | 297 | 61.670 | 65.912 | 157.681 | 1.00 | 0.00 | xxxx | 2287 |
| ATOM | 2288 | CG1 | ILE | A | 297 | 62.442 | 67.139 | 158.205 | 1.00 | 0.00 | xxxx | 2288 |
| ATOM | 2289 | CD1 | ILE | A | 297 | 63.572 | 66.805 | 159.187 | 1.00 | 0.00 | xxxx | 2289 |
| ATOM | 2290 | CG2 | ILE | A | 297 | 60.875 | 65.225 | 158.779 | 1.00 | 0.00 | xxxx | 2290 |
| ATOM | 2291 | C | ILE | A | 297 | 61.860 | 63.716 | 156.435 | 1.00 | 0.00 | xxxx | 2291 |
| ATOM | 2292 | O | ILE | A | 297 | 61.168 | 63.845 | 155.428 | 1.00 | 0.00 | xxxx | 2292 |
| ATOM | 2293 | N | PRO | A | 298 | 61.994 | 62.544 | 157.072 | 1.00 | 0.00 | xxxx | 2293 |
| ATOM | 2294 | CA | PRO | A | 298 | 61.335 | 61.350 | 156.526 | 1.00 | 0.00 | xxxx | 2294 |
| ATOM | 2295 | CB | PRO | A | 298 | 61.776 | 60.232 | 157.476 | 1.00 | 0.00 | xxxx | 2295 |
| ATOM | 2296 | CG | PRO | A | 298 | 63.030 | 60.727 | 158.091 | 1.00 | 0.00 | xxxx | 2296 |
| ATOM | 2297 | CD | PRO | A | 298 | 62.879 | 62.218 | 158.201 | 1.00 | 0.00 | xxxx | 2297 |
| ATOM | 2298 | C | PRO | A | 298 | 59.814 | 61.425 | 156.506 | 1.00 | 0.00 | xxxx | 2298 |
| ATOM | 2299 | O | PRO | A | 298 | 59.187 | 62.048 | 157.366 | 1.00 | 0.00 | xxxx | 2299 |
| ATOM | 2300 | N | TYR | A | 299 | 59.248 | 60.763 | 155.507 | 1.00 | 0.00 | xxxx | 2300 |
| ATOM | 2301 | CA | TYR | A | 299 | 57.828 | 60.450 | 155.457 | 1.00 | 0.00 | xxxx | 2301 |
| ATOM | 2302 | CB | TYR | A | 299 | 57.354 | 60.373 | 154.019 | 1.00 | 0.00 | xxxx | 2302 |
| ATOM | 2303 | CG | TYR | A | 299 | 57.128 | 61.683 | 153.320 | 1.00 | 0.00 | xxxx | 2303 |
| ATOM | 2304 | CD1 | TYR | A | 299 | 56.016 | 62.464 | 153.623 | 1.00 | 0.00 | xxxx | 2304 |
| ATOM | 2305 | CE1 | TYR | A | 299 | 55.781 | 63.651 | 152.964 | 1.00 | 0.00 | xxxx | 2305 |
| ATOM | 2306 | CZ | TYR | A | 299 | 56.642 | 64.066 | 151.973 | 1.00 | 0.00 | xxxx | 2306 |
| ATOM | 2307 | OH | TYR | A | 299 | 56.379 | 65.254 | 151.326 | 1.00 | 0.00 | xxxx | 2307 |
| ATOM | 2308 | CE2 | TYR | A | 299 | 57.753 | 63.298 | 151.630 | 1.00 | 0.00 | xxxx | 2308 |
| ATOM | 2309 | CD2 | TYR | A | 299 | 57.987 | 62.117 | 152.310 | 1.00 | 0.00 | xxxx | 2309 |
| ATOM | 2310 | C | TYR | A | 299 | 57.579 | 59.107 | 156.120 | 1.00 | 0.00 | xxxx | 2310 |
| ATOM | 2311 | O | TYR | A | 299 | 58.478 | 58.275 | 156.169 | 1.00 | 0.00 | xxxx | 2311 |
| ATOM | 2312 | N | LYS | A | 300 | 56.348 | 58.873 | 156.572 | 1.00 | 0.00 | xxxx | 2312 |
| ATOM | 2313 | CA | LYS | A | 300 | 55.993 | 57.607 | 157.212 | 1.00 | 0.00 | xxxx | 2313 |
| ATOM | 2314 | CB | LYS | A | 300 | 56.044 | 57.758 | 158.737 | 1.00 | 0.00 | xxxx | 2314 |
| ATOM | 2315 | CG | LYS | A | 300 | 55.632 | 56.512 | 159.500 | 1.00 | 0.00 | xxxx | 2315 |
| ATOM | 2316 | CD | LYS | A | 300 | 55.948 | 56.652 | 160.981 | 1.00 | 0.00 | xxxx | 2316 |
| ATOM | 2317 | CE | LYS | A | 300 | 55.732 | 55.342 | 161.715 | 1.00 | 0.00 | xxxx | 2317 |
| ATOM | 2318 | NZ | LYS | A | 300 | 56.541 | 54.228 | 161.143 | 1.00 | 0.00 | xxxx | 2318 |
| ATOM | 2319 | C | LYS | A | 300 | 54.601 | 57.129 | 156.772 | 1.00 | 0.00 | xxxx | 2319 |
| ATOM | 2320 | O | LYS | A | 300 | 53.637 | 57.899 | 156.808 | 1.00 | 0.00 | xxxx | 2320 |
| ATOM | 2321 | N | LYS | A | 301 | 54.493 | 55.862 | 156.376 | 1.00 | 0.00 | xxxx | 2321 |
| ATOM | 2322 | CA | LYS | A | 301 | 53.200 | 55.299 | 155.946 | 1.00 | 0.00 | xxxx | 2322 |
| ATOM | 2323 | CB | LYS | A | 301 | 53.409 | 53.910 | 155.323 | 1.00 | 0.00 | xxxx | 2323 |
| ATOM | 2324 | CG | LYS | A | 301 | 52.154 | 53.289 | 154.718 | 1.00 | 0.00 | xxxx | 2324 |
| ATOM | 2325 | CD | LYS | A | 301 | 52.372 | 51.812 | 154.360 | 1.00 | 0.00 | xxxx | 2325 |
| ATOM | 2326 | CE | LYS | A | 301 | 53.431 | 51.658 | 153.267 | 1.00 | 0.00 | xxxx | 2326 |
| ATOM | 2327 | NZ | LYS | A | 301 | 53.686 | 50.216 | 152.948 | 1.00 | 0.00 | xxxx | 2327 |
| ATOM | 2328 | C | LYS | A | 301 | 52.212 | 55.213 | 157.116 | 1.00 | 0.00 | xxxx | 2328 |
| ATOM | 2329 | O | LYS | A | 301 | 52.585 | 54.798 | 158.215 | 1.00 | 0.00 | xxxx | 2329 |
| ATOM | 2330 | N | ILE | A | 302 | 50.959 | 55.605 | 156.873 | 1.00 | 0.00 | xxxx | 2330 |
| ATOM | 2331 | CA | ILE | A | 302 | 49.900 | 55.566 | 157.892 | 1.00 | 0.00 | xxxx | 2331 |
| ATOM | 2332 | CB | ILE | A | 302 | 49.534 | 56.984 | 158.400 | 1.00 | 0.00 | xxxx | 2332 |
| ATOM | 2333 | CG1 | ILE | A | 302 | 50.755 | 57.717 | 158.975 | 1.00 | 0.00 | xxxx | 2333 |
| ATOM | 2334 | CD1 | ILE | A | 302 | 51.290 | 57.107 | 160.271 | 1.00 | 0.00 | xxxx | 2334 |
| ATOM | 2335 | CG2 | ILE | A | 302 | 48.379 | 56.906 | 159.406 | 1.00 | 0.00 | xxxx | 2335 |
| ATOM | 2336 | C | ILE | A | 302 | 48.642 | 54.893 | 157.348 | 1.00 | 0.00 | xxxx | 2336 |
| ATOM | 2337 | O | ILE | A | 302 | 48.058 | 55.349 | 156.350 | 1.00 | 0.00 | xxxx | 2337 |
| ATOM | 2338 | N | THR | A | 303 | 48.217 | 53.815 | 158.003 | 1.00 | 0.00 | xxxx | 2338 |
| ATOM | 2339 | CA | THR | A | 303 | 46.919 | 53.208 | 157.730 | 1.00 | 0.00 | xxxx | 2339 |
| ATOM | 2340 | CB | THR | A | 303 | 47.028 | 51.873 | 156.955 | 1.00 | 0.00 | xxxx | 2340 |
| ATOM | 2341 | OG1 | THR | A | 303 | 47.652 | 50.881 | 157.783 | 1.00 | 0.00 | xxxx | 2341 |
| ATOM | 2342 | CG2 | THR | A | 303 | 47.839 | 52.044 | 155.668 | 1.00 | 0.00 | xxxx | 2342 |
| ATOM | 2343 | C | THR | A | 303 | 46.224 | 52.959 | 159.061 | 1.00 | 0.00 | xxxx | 2343 |
| ATOM | 2344 | O | THR | A | 303 | 46.742 | 53.339 | 160.115 | 1.00 | 0.00 | xxxx | 2344 |
| ATOM | 2345 | N | LYS | A | 304 | 45.062 | 52.310 | 159.031 | 1.00 | 0.00 | xxxx | 2345 |
| ATOM | 2346 | CA | LYS | A | 304 | 44.365 | 52.034 | 160.284 | 1.00 | 0.00 | xxxx | 2346 |
| ATOM | 2347 | CB | LYS | A | 304 | 43.014 | 51.356 | 160.027 | 1.00 | 0.00 | xxxx | 2347 |
| ATOM | 2348 | CG | LYS | A | 304 | 43.113 | 49.981 | 159.388 | 1.00 | 0.00 | xxxx | 2348 |
| ATOM | 2349 | CD | LYS | A | 304 | 41.728 | 49.408 | 159.091 | 1.00 | 0.00 | xxxx | 2349 |
| ATOM | 2350 | CE | LYS | A | 304 | 41.817 | 48.045 | 158.422 | 1.00 | 0.00 | xxxx | 2350 |
| ATOM | 2351 | NZ | LYS | A | 304 | 40.513 | 47.632 | 157.829 | 1.00 | 0.00 | xxxx | 2351 |
| ATOM | 2352 | C | LYS | A | 304 | 45.224 | 51.168 | 161.207 | 1.00 | 0.00 | xxxx | 2352 |
| ATOM | 2353 | O | LYS | A | 304 | 45.055 | 51.198 | 162.423 | 1.00 | 0.00 | xxxx | 2353 |
| ATOM | 2354 | N | ASP | A | 305 | 46.162 | 50.421 | 160.626 | 1.00 | 0.00 | xxxx | 2354 |
| ATOM | 2355 | CA | ASP | A | 305 | 47.010 | 49.503 | 161.393 | 1.00 | 0.00 | xxxx | 2355 |
| ATOM | 2356 | CB | ASP | A | 305 | 47.778 | 48.575 | 160.446 | 1.00 | 0.00 | xxxx | 2356 |
| ATOM | 2357 | CG | ASP | A | 305 | 46.869 | 47.611 | 159.704 | 1.00 | 0.00 | xxxx | 2357 |
| ATOM | 2358 | OD1 | ASP | A | 305 | 45.789 | 47.276 | 160.236 | 1.00 | 0.00 | xxxx | 2358 |
| ATOM | 2359 | OD2 | ASP | A | 305 | 47.240 | 47.187 | 158.588 | 1.00 | 0.00 | xxxx | 2359 |
| ATOM | 2360 | C | ASP | A | 305 | 48.002 | 50.209 | 162.316 | 1.00 | 0.00 | xxxx | 2360 |
| ATOM | 2361 | O | ASP | A | 305 | 48.483 | 49.618 | 163.280 | 1.00 | 0.00 | xxxx | 2361 |
| ATOM | 2362 | N | ASN | A | 306 | 48.314 | 51.466 | 162.026 | 1.00 | 0.00 | xxxx | 2362 |
| ATOM | 2363 | CA | ASN | A | 306 | 49.287 | 52.189 | 162.835 | 1.00 | 0.00 | xxxx | 2363 |
| ATOM | 2364 | CB | ASN | A | 306 | 50.698 | 52.038 | 162.248 | 1.00 | 0.00 | xxxx | 2364 |
| ATOM | 2365 | CG | ASN | A | 306 | 50.854 | 52.713 | 160.891 | 1.00 | 0.00 | xxxx | 2365 |
| ATOM | 2366 | OD1 | ASN | A | 306 | 49.928 | 52.732 | 160.082 | 1.00 | 0.00 | xxxx | 2366 |
| ATOM | 2367 | ND2 | ASN | A | 306 | 52.037 | 53.261 | 160.637 | 1.00 | 0.00 | xxxx | 2367 |
| ATOM | 2368 | C | ASN | A | 306 | 48.918 | 53.660 | 162.969 | 1.00 | 0.00 | xxxx | 2368 |
| ATOM | 2369 | O | ASN | A | 306 | 49.786 | 54.536 | 162.985 | 1.00 | 0.00 | xxxx | 2369 |
| ATOM | 2370 | N | ILE | A | 307 | 47.618 | 53.920 | 163.088 | 1.00 | 0.00 | xxxx | 2370 |
| ATOM | 2371 | CA | ILE | A | 307 | 47.113 | 55.284 | 163.115 | 1.00 | 0.00 | xxxx | 2371 |
| ATOM | 2372 | CB | ILE | A | 307 | 45.566 | 55.285 | 163.204 | 1.00 | 0.00 | xxxx | 2372 |
| ATOM | 2373 | CG1 | ILE | A | 307 | 45.008 | 56.676 | 162.905 | 1.00 | 0.00 | xxxx | 2373 |
| ATOM | 2374 | CD1 | ILE | A | 307 | 45.401 | 57.215 | 161.543 | 1.00 | 0.00 | xxxx | 2374 |
| ATOM | 2375 | CG2 | ILE | A | 307 | 45.101 | 54.783 | 164.565 | 1.00 | 0.00 | xxxx | 2375 |
| ATOM | 2376 | C | ILE | A | 307 | 47.740 | 56.099 | 164.252 | 1.00 | 0.00 | xxxx | 2376 |
| ATOM | 2377 | O | ILE | A | 307 | 47.861 | 57.320 | 164.148 | 1.00 | 0.00 | xxxx | 2377 |
| ATOM | 2378 | N | SER | A | 308 | 48.172 | 55.431 | 165.320 | 1.00 | 0.00 | xxxx | 2378 |
| ATOM | 2379 | CA | SER | A | 308 | 48.767 | 56.141 | 166.452 | 1.00 | 0.00 | xxxx | 2379 |
| ATOM | 2380 | CB | SER | A | 308 | 48.875 | 55.219 | 167.673 | 1.00 | 0.00 | xxxx | 2380 |
| ATOM | 2381 | OG | SER | A | 308 | 49.886 | 54.245 | 167.496 | 1.00 | 0.00 | xxxx | 2381 |
| ATOM | 2382 | C | SER | A | 308 | 50.141 | 56.731 | 166.124 | 1.00 | 0.00 | xxxx | 2382 |
| ATOM | 2383 | O | SER | A | 308 | 50.586 | 57.664 | 166.790 | 1.00 | 0.00 | xxxx | 2383 |
| ATOM | 2384 | N | ASP | A | 309 | 50.805 | 56.205 | 165.095 | 1.00 | 0.00 | xxxx | 2384 |
| ATOM | 2385 | CA | ASP | A | 309 | 52.090 | 56.752 | 164.660 | 1.00 | 0.00 | xxxx | 2385 |
| ATOM | 2386 | CB | ASP | A | 309 | 52.726 | 55.882 | 163.569 | 1.00 | 0.00 | xxxx | 2386 |
| ATOM | 2387 | CG | ASP | A | 309 | 53.171 | 54.518 | 164.078 | 1.00 | 0.00 | xxxx | 2387 |
| ATOM | 2388 | OD1 | ASP | A | 309 | 53.127 | 54.286 | 165.307 | 1.00 | 0.00 | xxxx | 2388 |
| ATOM | 2389 | OD2 | ASP | A | 309 | 53.576 | 53.678 | 163.241 | 1.00 | 0.00 | xxxx | 2389 |
| ATOM | 2390 | C | ASP | A | 309 | 51.934 | 58.179 | 164.141 | 1.00 | 0.00 | xxxx | 2390 |
| ATOM | 2391 | O | ASP | A | 309 | 52.904 | 58.931 | 164.058 | 1.00 | 0.00 | xxxx | 2391 |
| ATOM | 2392 | N | ALA | A | 310 | 50.708 | 58.547 | 163.779 | 1.00 | 0.00 | xxxx | 2392 |
| ATOM | 2393 | CA | ALA | A | 310 | 50.434 | 59.898 | 163.299 | 1.00 | 0.00 | xxxx | 2393 |
| ATOM | 2394 | CB | ALA | A | 310 | 49.382 | 59.865 | 162.204 | 1.00 | 0.00 | xxxx | 2394 |
| ATOM | 2395 | C | ALA | A | 310 | 49.984 | 60.816 | 164.433 | 1.00 | 0.00 | xxxx | 2395 |
| ATOM | 2396 | O | ALA | A | 310 | 49.930 | 62.035 | 164.267 | 1.00 | 0.00 | xxxx | 2396 |
| ATOM | 2397 | N | GLU | A | 311 | 49.659 | 60.222 | 165.578 | 1.00 | 0.00 | xxxx | 2397 |
| ATOM | 2398 | CA | GLU | A | 311 | 49.189 | 60.972 | 166.740 | 1.00 | 0.00 | xxxx | 2398 |
| ATOM | 2399 | CB | GLU | A | 311 | 47.813 | 60.467 | 167.181 | 1.00 | 0.00 | xxxx | 2399 |
| ATOM | 2400 | CG | GLU | A | 311 | 46.695 | 60.765 | 166.192 | 1.00 | 0.00 | xxxx | 2400 |
| ATOM | 2401 | CD | GLU | A | 311 | 45.341 | 60.264 | 166.667 | 1.00 | 0.00 | xxxx | 2401 |
| ATOM | 2402 | OE1 | GLU | A | 311 | 45.230 | 59.066 | 167.007 | 1.00 | 0.00 | xxxx | 2402 |
| ATOM | 2403 | OE2 | GLU | A | 311 | 44.389 | 61.074 | 166.706 | 1.00 | 0.00 | xxxx | 2403 |
| ATOM | 2404 | C | GLU | A | 311 | 50.176 | 60.877 | 167.901 | 1.00 | 0.00 | xxxx | 2404 |
| ATOM | 2405 | O | GLU | A | 311 | 51.196 | 61.567 | 167.922 | 1.00 | 0.00 | xxxx | 2405 |
| HETATM | 2406 | CA | CA | B | 1 | 39.371 | 47.012 | 136.545 | 1.00 | 0.00 | xxxx | 2406 |
| HETATM | 2407 | C1 | GLC | D | 1 | 52.565 | 69.686 | 150.506 | 1.00 | 0.00 | xxxx | 2407 |
| HETATM | 2408 | O1 | GLC | D | 1 | 53.728 | 69.129 | 150.956 | 1.00 | 0.00 | xxxx | 2408 |
| HETATM | 2409 | C2 | GLC | D | 1 | 51.391 | 69.237 | 151.365 | 1.00 | 0.00 | xxxx | 2409 |
| HETATM | 2410 | O2 | GLC | D | 1 | 51.234 | 67.810 | 151.200 | 1.00 | 0.00 | xxxx | 2410 |
| HETATM | 2411 | C3 | GLC | D | 1 | 50.148 | 69.960 | 150.972 | 1.00 | 0.00 | xxxx | 2411 |
| HETATM | 2412 | O3 | GLC | D | 1 | 49.046 | 69.627 | 151.871 | 1.00 | 0.00 | xxxx | 2412 |
| HETATM | 2413 | C4 | GLC | D | 1 | 50.326 | 71.452 | 150.956 | 1.00 | 0.00 | xxxx | 2413 |
| HETATM | 2414 | O4 | GLC | D | 1 | 49.123 | 72.037 | 150.423 | 1.00 | 0.00 | xxxx | 2414 |
| HETATM | 2415 | C5 | GLC | D | 1 | 51.531 | 71.878 | 150.110 | 1.00 | 0.00 | xxxx | 2415 |
| HETATM | 2416 | O5 | GLC | D | 1 | 52.725 | 71.167 | 150.570 | 1.00 | 0.00 | xxxx | 2416 |
| HETATM | 2417 | C6 | GLC | D | 1 | 51.798 | 73.355 | 150.257 | 1.00 | 0.00 | xxxx | 2417 |
| HETATM | 2418 | O6 | GLC | D | 1 | 53.018 | 73.737 | 149.650 | 1.00 | 0.00 | xxxx | 2418 |
| HETATM | 2419 | K | K | E | 1 | 31.330 | 50.918 | 152.438 | 1.00 | 0.00 | xxxx | 2419 |
| HETATM | 2420 | O15 | BAD | H | 1 | 51.518 | 67.880 | 156.090 | 1.00 | 0.00 | xxxx | 2420 |
| HETATM | 2421 | C14 | BAD | H | 1 | 51.466 | 68.651 | 156.987 | 1.00 | 0.00 | xxxx | 2421 |
| HETATM | 2422 | C16 | BAD | H | 1 | 52.094 | 70.021 | 156.829 | 1.00 | 0.00 | xxxx | 2422 |
| HETATM | 2423 | C11 | BAD | H | 1 | 50.739 | 68.277 | 158.271 | 1.00 | 0.00 | xxxx | 2423 |
| HETATM | 2424 | C12 | BAD | H | 1 | 50.660 | 66.933 | 158.631 | 1.00 | 0.00 | xxxx | 2424 |
| HETATM | 2425 | C13 | BAD | H | 1 | 49.986 | 66.561 | 159.786 | 1.00 | 0.00 | xxxx | 2425 |
| HETATM | 2426 | C06 | BAD | H | 1 | 49.377 | 67.558 | 160.602 | 1.00 | 0.00 | xxxx | 2426 |
| HETATM | 2427 | C05 | BAD | H | 1 | 48.690 | 67.166 | 161.789 | 1.00 | 0.00 | xxxx | 2427 |
| HETATM | 2428 | C10 | BAD | H | 1 | 50.147 | 69.261 | 159.061 | 1.00 | 0.00 | xxxx | 2428 |
| HETATM | 2429 | C07 | BAD | H | 1 | 49.452 | 68.876 | 160.251 | 1.00 | 0.00 | xxxx | 2429 |
| HETATM | 2430 | C08 | BAD | H | 1 | 48.834 | 69.859 | 161.081 | 1.00 | 0.00 | xxxx | 2430 |
| HETATM | 2431 | C09 | BAD | H | 1 | 48.162 | 69.475 | 162.242 | 1.00 | 0.00 | xxxx | 2431 |
| HETATM | 2432 | C04 | BAD | H | 1 | 48.091 | 68.125 | 162.599 | 1.00 | 0.00 | xxxx | 2432 |
| HETATM | 2433 | N02 | BAD | H | 1 | 47.388 | 67.716 | 163.803 | 1.00 | 0.00 | xxxx | 2433 |
| HETATM | 2434 | C03 | BAD | H | 1 | 47.290 | 66.305 | 164.145 | 1.00 | 0.00 | xxxx | 2434 |
| HETATM | 2435 | C01 | BAD | H | 1 | 46.768 | 68.713 | 164.659 | 1.00 | 0.00 | xxxx | 2435 |
| HETATM | 2436 | O | HOH | S | 1 | 46.201 | 68.225 | 153.472 | 1.00 | 0.00 | xxxx | 2436 |
| HETATM | 2437 | O | HOH | S | 2 | 57.279 | 69.627 | 150.623 | 1.00 | 0.00 | xxxx | 2437 |
| HETATM | 2438 | O | HOH | S | 3 | 58.043 | 66.399 | 149.833 | 1.00 | 0.00 | xxxx | 2438 |
| HETATM | 2439 | O | HOH | S | 4 | 66.517 | 72.086 | 144.915 | 1.00 | 0.00 | xxxx | 2439 |
| HETATM | 2440 | O | HOH | S | 5 | 57.929 | 64.558 | 156.347 | 1.00 | 0.00 | xxxx | 2440 |
| HETATM | 2441 | O | HOH | S | 6 | 37.622 | 49.306 | 143.693 | 1.00 | 0.00 | xxxx | 2441 |
| HETATM | 2442 | O | HOH | S | 7 | 56.704 | 54.067 | 156.413 | 1.00 | 0.00 | xxxx | 2442 |
| HETATM | 2443 | O | HOH | S | 8 | 46.778 | 48.831 | 152.298 | 1.00 | 0.00 | xxxx | 2443 |
| HETATM | 2444 | O | HOH | S | 9 | 30.024 | 63.081 | 131.116 | 1.00 | 0.00 | xxxx | 2444 |
| HETATM | 2445 | O | HOH | S | 10 | 36.187 | 49.751 | 136.009 | 1.00 | 0.00 | xxxx | 2445 |
| HETATM | 2446 | O | HOH | S | 11 | 48.055 | 42.688 | 150.299 | 1.00 | 0.00 | xxxx | 2446 |
| HETATM | 2447 | O | HOH | S | 12 | 51.905 | 91.444 | 154.694 | 1.00 | 0.00 | xxxx | 2447 |
| HETATM | 2448 | O | HOH | S | 13 | 74.040 | 77.495 | 151.343 | 1.00 | 0.00 | xxxx | 2448 |
| HETATM | 2449 | O | HOH | S | 14 | 47.118 | 71.039 | 153.130 | 1.00 | 0.00 | xxxx | 2449 |
| HETATM | 2450 | O | HOH | S | 15 | 49.393 | 92.404 | 147.572 | 1.00 | 0.00 | xxxx | 2450 |
| HETATM | 2451 | O | HOH | S | 16 | 51.139 | 49.072 | 152.311 | 1.00 | 0.00 | xxxx | 2451 |
| HETATM | 2452 | O | HOH | S | 17 | 49.392 | 60.954 | 144.600 | 1.00 | 0.00 | xxxx | 2452 |
| HETATM | 2453 | O | HOH | S | 18 | 57.680 | 96.227 | 150.341 | 1.00 | 0.00 | xxxx | 2453 |
| HETATM | 2454 | O | HOH | S | 19 | 29.378 | 63.519 | 143.831 | 1.00 | 0.00 | xxxx | 2454 |
| HETATM | 2455 | O | HOH | S | 20 | 64.108 | 78.535 | 142.366 | 1.00 | 0.00 | xxxx | 2455 |
| HETATM | 2456 | O | HOH | S | 21 | 43.239 | 51.830 | 156.655 | 1.00 | 0.00 | xxxx | 2456 |
| HETATM | 2457 | O | HOH | S | 22 | 39.137 | 53.223 | 153.258 | 1.00 | 0.00 | xxxx | 2457 |
| HETATM | 2458 | O | HOH | S | 23 | 40.641 | 53.517 | 156.840 | 1.00 | 0.00 | xxxx | 2458 |
| HETATM | 2459 | O | HOH | S | 24 | 75.860 | 67.970 | 153.435 | 1.00 | 0.00 | xxxx | 2459 |
| HETATM | 2460 | O | HOH | S | 25 | 60.885 | 68.326 | 148.044 | 1.00 | 0.00 | xxxx | 2460 |
| HETATM | 2461 | O | HOH | S | 26 | 56.455 | 69.597 | 153.366 | 1.00 | 0.00 | xxxx | 2461 |
| HETATM | 2462 | O | HOH | S | 27 | 49.093 | 78.034 | 156.339 | 1.00 | 0.00 | xxxx | 2462 |
| HETATM | 2463 | O | HOH | S | 28 | 51.534 | 84.253 | 141.942 | 1.00 | 0.00 | xxxx | 2463 |
| HETATM | 2464 | O | HOH | S | 29 | 41.409 | 72.967 | 151.410 | 1.00 | 0.00 | xxxx | 2464 |
| HETATM | 2465 | O | HOH | S | 30 | 52.391 | 92.584 | 150.793 | 1.00 | 0.00 | xxxx | 2465 |
| HETATM | 2466 | O | HOH | S | 31 | 49.882 | 88.636 | 156.756 | 1.00 | 0.00 | xxxx | 2466 |
| HETATM | 2467 | O | HOH | S | 32 | 49.907 | 49.386 | 133.465 | 1.00 | 0.00 | xxxx | 2467 |
| HETATM | 2468 | O | HOH | S | 33 | 51.656 | 93.752 | 148.385 | 1.00 | 0.00 | xxxx | 2468 |
| HETATM | 2469 | O | HOH | S | 34 | 46.499 | 77.583 | 143.348 | 1.00 | 0.00 | xxxx | 2469 |
| HETATM | 2470 | O | HOH | S | 35 | 47.019 | 43.680 | 137.225 | 1.00 | 0.00 | xxxx | 2470 |
| HETATM | 2471 | O | HOH | S | 36 | 62.183 | 62.671 | 153.207 | 1.00 | 0.00 | xxxx | 2471 |
| HETATM | 2472 | O | HOH | S | 37 | 54.270 | 92.901 | 154.716 | 1.00 | 0.00 | xxxx | 2472 |
| HETATM | 2473 | O | HOH | S | 38 | 54.221 | 63.904 | 137.446 | 1.00 | 0.00 | xxxx | 2473 |
| HETATM | 2474 | O | HOH | S | 39 | 41.749 | 75.482 | 143.484 | 1.00 | 0.00 | xxxx | 2474 |
| HETATM | 2475 | O | HOH | S | 40 | 38.789 | 74.147 | 149.924 | 1.00 | 0.00 | xxxx | 2475 |
| HETATM | 2476 | O | HOH | S | 41 | 57.442 | 50.553 | 153.136 | 1.00 | 0.00 | xxxx | 2476 |
| HETATM | 2477 | O | HOH | S | 42 | 45.951 | 75.220 | 138.761 | 1.00 | 0.00 | xxxx | 2477 |
| HETATM | 2478 | O | HOH | S | 43 | 38.706 | 67.514 | 135.380 | 1.00 | 0.00 | xxxx | 2478 |
| HETATM | 2479 | O | HOH | S | 44 | 68.866 | 91.816 | 177.553 | 1.00 | 0.00 | xxxx | 2479 |
| HETATM | 2480 | O | HOH | S | 45 | 50.702 | 42.893 | 149.907 | 1.00 | 0.00 | xxxx | 2480 |
| HETATM | 2481 | O | HOH | S | 46 | 74.261 | 65.933 | 144.734 | 1.00 | 0.00 | xxxx | 2481 |
| HETATM | 2482 | O | HOH | S | 47 | 55.766 | 48.358 | 144.532 | 1.00 | 0.00 | xxxx | 2482 |
| HETATM | 2483 | O | HOH | S | 48 | 54.068 | 47.601 | 151.452 | 1.00 | 0.00 | xxxx | 2483 |
| HETATM | 2484 | O | HOH | S | 49 | 36.924 | 48.495 | 146.281 | 1.00 | 0.00 | xxxx | 2484 |
| HETATM | 2485 | O | HOH | S | 50 | 28.577 | 63.305 | 141.186 | 1.00 | 0.00 | xxxx | 2485 |
| HETATM | 2486 | O | HOH | S | 51 | 56.380 | 51.624 | 155.427 | 1.00 | 0.00 | xxxx | 2486 |
| HETATM | 2487 | O | HOH | S | 52 | 46.843 | 76.105 | 147.173 | 1.00 | 0.00 | xxxx | 2487 |
| HETATM | 2488 | O | HOH | S | 53 | 54.201 | 94.196 | 152.355 | 1.00 | 0.00 | xxxx | 2488 |
| HETATM | 2489 | O | HOH | S | 54 | 36.411 | 66.940 | 136.708 | 1.00 | 0.00 | xxxx | 2489 |
| HETATM | 2490 | O | HOH | S | 55 | 34.508 | 64.230 | 133.579 | 1.00 | 0.00 | xxxx | 2490 |
| HETATM | 2491 | O | HOH | S | 56 | 37.469 | 75.613 | 143.245 | 1.00 | 0.00 | xxxx | 2491 |
| HETATM | 2492 | O | HOH | S | 57 | 41.259 | 63.933 | 132.633 | 1.00 | 0.00 | xxxx | 2492 |
| HETATM | 2493 | O | HOH | S | 58 | 56.874 | 61.551 | 158.772 | 1.00 | 0.00 | xxxx | 2493 |
| HETATM | 2494 | O | HOH | S | 59 | 56.894 | 82.352 | 168.913 | 1.00 | 0.00 | xxxx | 2494 |
| HETATM | 2495 | O | HOH | S | 60 | 52.215 | 45.595 | 146.666 | 1.00 | 0.00 | xxxx | 2495 |
| HETATM | 2496 | O | HOH | S | 61 | 69.476 | 66.394 | 148.256 | 1.00 | 0.00 | xxxx | 2496 |
| HETATM | 2497 | O | HOH | S | 62 | 44.078 | 67.855 | 155.344 | 1.00 | 0.00 | xxxx | 2497 |
| HETATM | 2498 | O | HOH | S | 63 | 37.822 | 64.070 | 158.296 | 1.00 | 0.00 | xxxx | 2498 |
| HETATM | 2499 | O | HOH | S | 64 | 31.238 | 72.940 | 145.995 | 1.00 | 0.00 | xxxx | 2499 |
| HETATM | 2500 | O | HOH | S | 65 | 58.159 | 86.918 | 140.884 | 1.00 | 0.00 | xxxx | 2500 |
| HETATM | 2501 | O | HOH | S | 66 | 31.369 | 69.344 | 139.912 | 1.00 | 0.00 | xxxx | 2501 |
| HETATM | 2502 | O | HOH | S | 67 | 75.699 | 79.234 | 155.569 | 1.00 | 0.00 | xxxx | 2502 |
| HETATM | 2503 | O | HOH | S | 68 | 74.962 | 66.801 | 158.209 | 1.00 | 0.00 | xxxx | 2503 |
| HETATM | 2504 | O | HOH | S | 69 | 61.668 | 65.708 | 146.688 | 1.00 | 0.00 | xxxx | 2504 |
| HETATM | 2505 | O | HOH | S | 70 | 47.651 | 39.922 | 149.628 | 1.00 | 0.00 | xxxx | 2505 |
| HETATM | 2506 | O | HOH | S | 71 | 50.222 | 79.601 | 143.279 | 1.00 | 0.00 | xxxx | 2506 |
| HETATM | 2507 | O | HOH | S | 72 | 36.280 | 50.638 | 133.281 | 1.00 | 0.00 | xxxx | 2507 |
| HETATM | 2508 | O | HOH | S | 73 | 56.688 | 91.754 | 155.167 | 1.00 | 0.00 | xxxx | 2508 |
| HETATM | 2509 | O | HOH | S | 74 | 56.515 | 91.297 | 141.991 | 1.00 | 0.00 | xxxx | 2509 |
| HETATM | 2510 | O | HOH | S | 75 | 60.126 | 97.716 | 151.243 | 1.00 | 0.00 | xxxx | 2510 |
| HETATM | 2511 | O | HOH | S | 76 | 59.113 | 72.275 | 155.191 | 1.00 | 0.00 | xxxx | 2511 |
| HETATM | 2512 | O | HOH | S | 77 | 39.286 | 65.407 | 133.676 | 1.00 | 0.00 | xxxx | 2512 |
| HETATM | 2513 | O | HOH | S | 78 | 51.850 | 77.610 | 168.352 | 1.00 | 0.00 | xxxx | 2513 |
| HETATM | 2514 | O | HOH | S | 79 | 63.904 | 96.103 | 150.019 | 1.00 | 0.00 | xxxx | 2514 |
| HETATM | 2515 | O | HOH | S | 80 | 30.586 | 65.593 | 134.386 | 1.00 | 0.00 | xxxx | 2515 |
| HETATM | 2516 | O | HOH | S | 81 | 42.210 | 63.701 | 164.172 | 1.00 | 0.00 | xxxx | 2516 |
| HETATM | 2517 | O | HOH | S | 82 | 58.490 | 52.146 | 143.356 | 1.00 | 0.00 | xxxx | 2517 |
| HETATM | 2518 | O | HOH | S | 83 | 35.827 | 64.052 | 153.949 | 1.00 | 0.00 | xxxx | 2518 |
| HETATM | 2519 | O | HOH | S | 84 | 51.916 | 66.901 | 138.432 | 1.00 | 0.00 | xxxx | 2519 |
| HETATM | 2520 | O | HOH | S | 85 | 45.093 | 72.744 | 152.280 | 1.00 | 0.00 | xxxx | 2520 |
| HETATM | 2521 | O | HOH | S | 86 | 55.336 | 96.534 | 151.494 | 1.00 | 0.00 | xxxx | 2521 |
| HETATM | 2522 | O | HOH | S | 87 | 54.897 | 53.328 | 158.820 | 1.00 | 0.00 | xxxx | 2522 |
| HETATM | 2523 | O | HOH | S | 88 | 51.214 | 76.347 | 140.158 | 1.00 | 0.00 | xxxx | 2523 |
| HETATM | 2524 | O | HOH | S | 89 | 76.016 | 74.651 | 156.563 | 1.00 | 0.00 | xxxx | 2524 |
| HETATM | 2525 | O | HOH | S | 90 | 76.998 | 77.686 | 147.743 | 1.00 | 0.00 | xxxx | 2525 |
| HETATM | 2526 | O | HOH | S | 91 | 45.905 | 86.694 | 146.512 | 1.00 | 0.00 | xxxx | 2526 |
| HETATM | 2527 | O | HOH | S | 92 | 49.430 | 92.090 | 144.568 | 1.00 | 0.00 | xxxx | 2527 |
| HETATM | 2528 | O | HOH | S | 93 | 39.674 | 61.796 | 131.497 | 1.00 | 0.00 | xxxx | 2528 |
| HETATM | 2529 | O | HOH | S | 94 | 55.245 | 99.007 | 153.139 | 1.00 | 0.00 | xxxx | 2529 |
| HETATM | 2530 | O | HOH | S | 95 | 69.244 | 63.350 | 148.315 | 1.00 | 0.00 | xxxx | 2530 |
| HETATM | 2531 | O | HOH | S | 96 | 62.369 | 67.500 | 144.662 | 1.00 | 0.00 | xxxx | 2531 |
| HETATM | 2532 | O | HOH | S | 97 | 51.637 | 42.869 | 152.661 | 1.00 | 0.00 | xxxx | 2532 |
| HETATM | 2533 | O | HOH | S | 98 | 53.212 | 61.591 | 136.064 | 1.00 | 0.00 | xxxx | 2533 |
| HETATM | 2534 | O | HOH | S | 99 | 66.212 | 62.210 | 156.589 | 1.00 | 0.00 | xxxx | 2534 |
| HETATM | 2535 | O | HOH | S | 100 | 35.206 | 69.485 | 136.668 | 1.00 | 0.00 | xxxx | 2535 |
| HETATM | 2536 | O | HOH | S | 101 | 67.877 | 74.242 | 138.545 | 1.00 | 0.00 | xxxx | 2536 |
| HETATM | 2537 | O | HOH | S | 102 | 47.256 | 88.958 | 147.564 | 1.00 | 0.00 | xxxx | 2537 |
| HETATM | 2538 | O | HOH | S | 103 | 48.802 | 89.549 | 152.888 | 1.00 | 0.00 | xxxx | 2538 |
| HETATM | 2539 | O | HOH | S | 104 | 55.533 | 47.093 | 149.026 | 1.00 | 0.00 | xxxx | 2539 |
| HETATM | 2540 | O | HOH | S | 105 | 58.683 | 84.568 | 172.531 | 1.00 | 0.00 | xxxx | 2540 |
| HETATM | 2541 | O | HOH | S | 106 | 46.755 | 54.806 | 131.997 | 1.00 | 0.00 | xxxx | 2541 |
| HETATM | 2542 | O | HOH | S | 107 | 79.055 | 76.628 | 162.628 | 1.00 | 0.00 | xxxx | 2542 |
| HETATM | 2543 | O | HOH | S | 108 | 35.822 | 74.142 | 141.537 | 1.00 | 0.00 | xxxx | 2543 |
| HETATM | 2544 | O | HOH | S | 109 | 50.310 | 81.760 | 142.022 | 1.00 | 0.00 | xxxx | 2544 |
| HETATM | 2545 | O | HOH | S | 110 | 63.805 | 83.471 | 140.207 | 1.00 | 0.00 | xxxx | 2545 |
| HETATM | 2546 | O | HOH | S | 111 | 27.119 | 60.930 | 140.514 | 1.00 | 0.00 | xxxx | 2546 |
| HETATM | 2547 | O | HOH | S | 112 | 57.702 | 49.618 | 146.404 | 1.00 | 0.00 | xxxx | 2547 |
| HETATM | 2548 | O | HOH | S | 113 | 53.156 | 41.009 | 142.253 | 1.00 | 0.00 | xxxx | 2548 |
| HETATM | 2549 | O | HOH | S | 114 | 60.853 | 82.005 | 137.338 | 1.00 | 0.00 | xxxx | 2549 |
| HETATM | 2550 | O | HOH | S | 115 | 65.759 | 67.945 | 145.250 | 1.00 | 0.00 | xxxx | 2550 |
| HETATM | 2551 | O | HOH | S | 116 | 79.693 | 79.535 | 162.418 | 1.00 | 0.00 | xxxx | 2551 |
| HETATM | 2552 | O | HOH | S | 117 | 42.135 | 47.615 | 154.138 | 1.00 | 0.00 | xxxx | 2552 |
| HETATM | 2553 | O | HOH | S | 118 | 57.040 | 63.514 | 136.108 | 1.00 | 0.00 | xxxx | 2553 |
| HETATM | 2554 | O | HOH | S | 119 | 31.769 | 48.527 | 146.542 | 1.00 | 0.00 | xxxx | 2554 |
| HETATM | 2555 | O | HOH | S | 120 | 57.039 | 84.513 | 139.915 | 1.00 | 0.00 | xxxx | 2555 |
| HETATM | 2556 | O | HOH | S | 121 | 46.159 | 42.652 | 139.578 | 1.00 | 0.00 | xxxx | 2556 |
| HETATM | 2557 | O | HOH | S | 122 | 53.925 | 44.527 | 149.407 | 1.00 | 0.00 | xxxx | 2557 |
| HETATM | 2558 | O | HOH | S | 123 | 66.892 | 67.604 | 166.801 | 1.00 | 0.00 | xxxx | 2558 |
| HETATM | 2559 | O | HOH | S | 124 | 35.159 | 66.646 | 134.153 | 1.00 | 0.00 | xxxx | 2559 |
| HETATM | 2560 | O | HOH | S | 125 | 78.744 | 72.675 | 165.106 | 1.00 | 0.00 | xxxx | 2560 |
| HETATM | 2561 | O | HOH | S | 126 | 76.832 | 75.603 | 145.788 | 1.00 | 0.00 | xxxx | 2561 |
| HETATM | 2562 | O | HOH | S | 127 | 54.214 | 57.362 | 136.743 | 1.00 | 0.00 | xxxx | 2562 |
| HETATM | 2563 | O | HOH | S | 128 | 53.898 | 47.656 | 146.752 | 1.00 | 0.00 | xxxx | 2563 |
| HETATM | 2564 | O | HOH | S | 129 | 49.728 | 63.948 | 162.353 | 1.00 | 0.00 | xxxx | 2564 |
| HETATM | 2565 | O | HOH | S | 130 | 57.315 | 85.289 | 167.511 | 1.00 | 0.00 | xxxx | 2565 |
| HETATM | 2566 | O | HOH | S | 131 | 72.797 | 63.380 | 153.609 | 1.00 | 0.00 | xxxx | 2566 |
| HETATM | 2567 | O | HOH | S | 132 | 55.428 | 92.803 | 143.759 | 1.00 | 0.00 | xxxx | 2567 |
| HETATM | 2568 | O | HOH | S | 133 | 68.279 | 93.604 | 148.640 | 1.00 | 0.00 | xxxx | 2568 |
| HETATM | 2569 | O | HOH | S | 134 | 76.091 | 89.391 | 150.922 | 1.00 | 0.00 | xxxx | 2569 |
| HETATM | 2570 | O | HOH | S | 135 | 45.377 | 74.405 | 150.380 | 1.00 | 0.00 | xxxx | 2570 |
| HETATM | 2571 | O | HOH | S | 136 | 44.404 | 77.789 | 145.325 | 1.00 | 0.00 | xxxx | 2571 |
| HETATM | 2572 | O | HOH | S | 137 | 45.325 | 74.050 | 136.311 | 1.00 | 0.00 | xxxx | 2572 |
| HETATM | 2573 | O | HOH | S | 138 | 73.690 | 79.101 | 140.386 | 1.00 | 0.00 | xxxx | 2573 |
| HETATM | 2574 | O | HOH | S | 139 | 52.155 | 93.059 | 144.447 | 1.00 | 0.00 | xxxx | 2574 |
| HETATM | 2575 | O | HOH | S | 140 | 40.016 | 69.933 | 155.465 | 1.00 | 0.00 | xxxx | 2575 |
| HETATM | 2576 | O | HOH | S | 141 | 50.687 | 90.990 | 157.036 | 1.00 | 0.00 | xxxx | 2576 |
| HETATM | 2577 | O | HOH | S | 142 | 32.692 | 60.138 | 157.561 | 1.00 | 0.00 | xxxx | 2577 |
| HETATM | 2578 | O | HOH | S | 143 | 37.136 | 46.283 | 149.886 | 1.00 | 0.00 | xxxx | 2578 |
| HETATM | 2579 | O | HOH | S | 144 | 64.760 | 94.189 | 148.540 | 1.00 | 0.00 | xxxx | 2579 |
| HETATM | 2580 | O | HOH | S | 145 | 44.949 | 83.318 | 153.400 | 1.00 | 0.00 | xxxx | 2580 |
| HETATM | 2581 | O | HOH | S | 146 | 49.102 | 45.603 | 154.088 | 1.00 | 0.00 | xxxx | 2581 |
| HETATM | 2582 | O | HOH | S | 147 | 50.741 | 82.205 | 161.982 | 1.00 | 0.00 | xxxx | 2582 |
| HETATM | 2583 | O | HOH | S | 148 | 45.534 | 91.850 | 151.024 | 1.00 | 0.00 | xxxx | 2583 |
| HETATM | 2584 | O | HOH | S | 149 | 26.998 | 57.738 | 151.095 | 1.00 | 0.00 | xxxx | 2584 |
| HETATM | 2585 | O | HOH | S | 150 | 57.998 | 96.596 | 147.563 | 1.00 | 0.00 | xxxx | 2585 |
| HETATM | 2586 | O | HOH | S | 151 | 54.046 | 55.078 | 135.707 | 1.00 | 0.00 | xxxx | 2586 |
| HETATM | 2587 | O | HOH | S | 152 | 36.107 | 62.034 | 158.248 | 1.00 | 0.00 | xxxx | 2587 |
| HETATM | 2588 | O | HOH | S | 153 | 69.880 | 93.089 | 164.909 | 1.00 | 0.00 | xxxx | 2588 |
| HETATM | 2589 | O | HOH | S | 154 | 44.962 | 55.765 | 130.418 | 1.00 | 0.00 | xxxx | 2589 |
| HETATM | 2590 | O | HOH | S | 155 | 69.382 | 76.162 | 166.572 | 1.00 | 0.00 | xxxx | 2590 |
| HETATM | 2591 | O | HOH | S | 156 | 48.993 | 48.521 | 153.991 | 1.00 | 0.00 | xxxx | 2591 |
| HETATM | 2592 | O | HOH | S | 157 | 42.538 | 75.186 | 150.705 | 1.00 | 0.00 | xxxx | 2592 |
| HETATM | 2593 | O | HOH | S | 158 | 34.529 | 47.526 | 131.504 | 1.00 | 0.00 | xxxx | 2593 |
| HETATM | 2594 | O | HOH | S | 159 | 68.597 | 95.583 | 154.648 | 1.00 | 0.00 | xxxx | 2594 |
| HETATM | 2595 | O | HOH | S | 160 | 47.611 | 89.569 | 155.230 | 1.00 | 0.00 | xxxx | 2595 |
| HETATM | 2596 | O | HOH | S | 161 | 41.586 | 43.733 | 142.934 | 1.00 | 0.00 | xxxx | 2596 |
| HETATM | 2597 | O | HOH | S | 162 | 67.759 | 84.003 | 171.737 | 1.00 | 0.00 | xxxx | 2597 |
| HETATM | 2598 | O | HOH | S | 163 | 34.711 | 71.043 | 138.594 | 1.00 | 0.00 | xxxx | 2598 |
| HETATM | 2599 | O | HOH | S | 164 | 32.185 | 67.192 | 133.433 | 1.00 | 0.00 | xxxx | 2599 |
| HETATM | 2600 | O | HOH | S | 165 | 64.999 | 65.696 | 145.876 | 1.00 | 0.00 | xxxx | 2600 |
| HETATM | 2601 | O | HOH | S | 166 | 56.782 | 64.948 | 164.863 | 1.00 | 0.00 | xxxx | 2601 |
| HETATM | 2602 | O | HOH | S | 167 | 72.553 | 89.540 | 144.762 | 1.00 | 0.00 | xxxx | 2602 |
| HETATM | 2603 | O | HOH | S | 168 | 53.053 | 66.089 | 136.571 | 1.00 | 0.00 | xxxx | 2603 |
| HETATM | 2604 | O | HOH | S | 169 | 52.271 | 72.917 | 167.041 | 1.00 | 0.00 | xxxx | 2604 |
| HETATM | 2605 | O | HOH | S | 170 | 70.989 | 61.973 | 155.873 | 1.00 | 0.00 | xxxx | 2605 |
| HETATM | 2606 | O | HOH | S | 171 | 48.753 | 73.251 | 164.610 | 1.00 | 0.00 | xxxx | 2606 |
| HETATM | 2607 | O | HOH | S | 172 | 51.477 | 45.150 | 153.501 | 1.00 | 0.00 | xxxx | 2607 |
| HETATM | 2608 | O | HOH | S | 173 | 70.613 | 93.728 | 160.943 | 1.00 | 0.00 | xxxx | 2608 |
| HETATM | 2609 | O | HOH | S | 174 | 51.891 | 92.250 | 158.877 | 1.00 | 0.00 | xxxx | 2609 |
| HETATM | 2610 | O | HOH | S | 175 | 48.940 | 42.197 | 136.314 | 1.00 | 0.00 | xxxx | 2610 |
| HETATM | 2611 | O | HOH | S | 176 | 68.735 | 63.111 | 155.715 | 1.00 | 0.00 | xxxx | 2611 |
| HETATM | 2612 | O | HOH | S | 177 | 60.769 | 62.992 | 166.104 | 1.00 | 0.00 | xxxx | 2612 |
| HETATM | 2613 | O | HOH | S | 178 | 50.187 | 93.478 | 153.677 | 1.00 | 0.00 | xxxx | 2613 |
| HETATM | 2614 | O | HOH | S | 179 | 51.213 | 80.674 | 164.196 | 1.00 | 0.00 | xxxx | 2614 |
| HETATM | 2615 | O | HOH | S | 180 | 49.537 | 49.269 | 156.623 | 1.00 | 0.00 | xxxx | 2615 |
| HETATM | 2616 | O | HOH | S | 181 | 77.139 | 80.622 | 157.215 | 1.00 | 0.00 | xxxx | 2616 |
| HETATM | 2617 | O | HOH | S | 182 | 71.895 | 90.577 | 158.371 | 1.00 | 0.00 | xxxx | 2617 |
| HETATM | 2618 | O | HOH | S | 183 | 49.979 | 79.111 | 167.457 | 1.00 | 0.00 | xxxx | 2618 |
| HETATM | 2619 | O | HOH | S | 184 | 39.460 | 72.232 | 154.081 | 1.00 | 0.00 | xxxx | 2619 |
| HETATM | 2620 | O | HOH | S | 185 | 57.704 | 47.790 | 153.481 | 1.00 | 0.00 | xxxx | 2620 |
| HETATM | 2621 | O | HOH | S | 186 | 36.677 | 72.491 | 139.643 | 1.00 | 0.00 | xxxx | 2621 |
| HETATM | 2622 | O | HOH | S | 187 | 71.999 | 61.960 | 151.927 | 1.00 | 0.00 | xxxx | 2622 |
| HETATM | 2623 | O | HOH | S | 188 | 47.075 | 72.908 | 162.806 | 1.00 | 0.00 | xxxx | 2623 |
| HETATM | 2624 | O | HOH | S | 189 | 35.414 | 76.807 | 144.818 | 1.00 | 0.00 | xxxx | 2624 |
| HETATM | 2625 | O | HOH | S | 190 | 78.789 | 76.672 | 149.395 | 1.00 | 0.00 | xxxx | 2625 |
| HETATM | 2626 | O | HOH | S | 191 | 60.444 | 53.912 | 142.416 | 1.00 | 0.00 | xxxx | 2626 |
| HETATM | 2627 | O | HOH | S | 192 | 42.137 | 43.424 | 130.368 | 1.00 | 0.00 | xxxx | 2627 |
| HETATM | 2628 | O | HOH | S | 193 | 40.114 | 41.774 | 129.906 | 1.00 | 0.00 | xxxx | 2628 |
| HETATM | 2629 | O | HOH | S | 194 | 60.042 | 77.391 | 172.458 | 1.00 | 0.00 | xxxx | 2629 |
| HETATM | 2630 | O | HOH | S | 195 | 58.805 | 47.855 | 156.024 | 1.00 | 0.00 | xxxx | 2630 |
| HETATM | 2631 | O | HOH | S | 196 | 60.005 | 50.731 | 150.338 | 1.00 | 0.00 | xxxx | 2631 |
| HETATM | 2632 | O | HOH | S | 197 | 59.841 | 61.910 | 160.341 | 1.00 | 0.00 | xxxx | 2632 |
| HETATM | 2633 | O | HOH | S | 198 | 77.400 | 74.129 | 149.989 | 1.00 | 0.00 | xxxx | 2633 |
| HETATM | 2634 | O | HOH | S | 199 | 53.896 | 98.937 | 155.309 | 1.00 | 0.00 | xxxx | 2634 |
| HETATM | 2635 | O | HOH | S | 200 | 59.636 | 83.798 | 138.796 | 1.00 | 0.00 | xxxx | 2635 |
| HETATM | 2636 | O | HOH | S | 201 | 32.247 | 43.843 | 140.408 | 1.00 | 0.00 | xxxx | 2636 |
| HETATM | 2637 | O | HOH | S | 202 | 29.486 | 64.466 | 155.083 | 1.00 | 0.00 | xxxx | 2637 |
| HETATM | 2638 | O | HOH | S | 203 | 42.716 | 72.150 | 135.089 | 1.00 | 0.00 | xxxx | 2638 |
| HETATM | 2639 | O | HOH | S | 204 | 71.987 | 88.136 | 159.671 | 1.00 | 0.00 | xxxx | 2639 |
| HETATM | 2640 | O | HOH | S | 205 | 33.381 | 49.010 | 136.893 | 1.00 | 0.00 | xxxx | 2640 |
| HETATM | 2641 | O | HOH | S | 206 | 69.194 | 95.620 | 149.891 | 1.00 | 0.00 | xxxx | 2641 |
| HETATM | 2642 | O | HOH | S | 207 | 29.387 | 55.844 | 141.016 | 1.00 | 0.00 | xxxx | 2642 |
| HETATM | 2643 | O | HOH | S | 208 | 45.490 | 92.898 | 153.531 | 1.00 | 0.00 | xxxx | 2643 |
| HETATM | 2644 | O | HOH | S | 209 | 78.479 | 75.596 | 143.871 | 1.00 | 0.00 | xxxx | 2644 |
| HETATM | 2645 | O | HOH | S | 210 | 54.709 | 51.509 | 162.804 | 1.00 | 0.00 | xxxx | 2645 |
| HETATM | 2646 | O | HOH | S | 211 | 48.561 | 89.863 | 144.071 | 1.00 | 0.00 | xxxx | 2646 |
| HETATM | 2647 | O | HOH | S | 212 | 39.863 | 48.853 | 153.284 | 1.00 | 0.00 | xxxx | 2647 |
| HETATM | 2648 | O | HOH | S | 213 | 80.062 | 74.843 | 164.569 | 1.00 | 0.00 | xxxx | 2648 |
| HETATM | 2649 | O | HOH | S | 214 | 38.835 | 54.728 | 150.936 | 1.00 | 0.00 | xxxx | 2649 |
| HETATM | 2650 | O | HOH | S | 215 | 68.967 | 65.864 | 155.242 | 1.00 | 0.00 | xxxx | 2650 |
| HETATM | 2651 | O | HOH | S | 216 | 68.172 | 67.122 | 145.858 | 1.00 | 0.00 | xxxx | 2651 |
| HETATM | 2652 | O | HOH | S | 217 | 68.298 | 64.720 | 144.670 | 1.00 | 0.00 | xxxx | 2652 |
| HETATM | 2653 | O | HOH | S | 218 | 71.593 | 72.342 | 144.610 | 1.00 | 0.00 | xxxx | 2653 |
| HETATM | 2654 | O | HOH | S | 219 | 64.396 | 61.473 | 153.855 | 1.00 | 0.00 | xxxx | 2654 |
| HETATM | 2655 | O | HOH | S | 220 | 55.312 | 96.339 | 144.330 | 1.00 | 0.00 | xxxx | 2655 |
| HETATM | 2656 | O | HOH | S | 221 | 54.183 | 48.804 | 155.351 | 1.00 | 0.00 | xxxx | 2656 |
| HETATM | 2657 | O | HOH | S | 222 | 74.731 | 90.692 | 145.593 | 1.00 | 0.00 | xxxx | 2657 |
| HETATM | 2658 | O | HOH | S | 223 | 37.467 | 65.926 | 160.398 | 1.00 | 0.00 | xxxx | 2658 |
| HETATM | 2659 | O | HOH | S | 224 | 27.727 | 58.401 | 140.473 | 1.00 | 0.00 | xxxx | 2659 |
| HETATM | 2660 | O | HOH | S | 225 | 43.576 | 85.936 | 150.480 | 1.00 | 0.00 | xxxx | 2660 |
| HETATM | 2661 | O | HOH | S | 226 | 45.728 | 86.581 | 156.293 | 1.00 | 0.00 | xxxx | 2661 |
| HETATM | 2662 | O | HOH | S | 227 | 49.995 | 92.055 | 151.996 | 1.00 | 0.00 | xxxx | 2662 |
| HETATM | 2663 | O | HOH | S | 228 | 37.499 | 47.113 | 152.538 | 1.00 | 0.00 | xxxx | 2663 |
| HETATM | 2664 | O | HOH | S | 229 | 44.424 | 61.222 | 132.009 | 1.00 | 0.00 | xxxx | 2664 |
| HETATM | 2665 | O | HOH | S | 230 | 76.652 | 68.937 | 159.384 | 1.00 | 0.00 | xxxx | 2665 |
| HETATM | 2666 | O | HOH | S | 231 | 40.922 | 61.562 | 164.902 | 1.00 | 0.00 | xxxx | 2666 |
| HETATM | 2667 | O | HOH | S | 232 | 64.719 | 94.729 | 167.844 | 1.00 | 0.00 | xxxx | 2667 |
| HETATM | 2668 | O | HOH | S | 233 | 43.542 | 59.604 | 131.071 | 1.00 | 0.00 | xxxx | 2668 |
| HETATM | 2669 | O | HOH | S | 234 | 51.200 | 63.892 | 135.249 | 1.00 | 0.00 | xxxx | 2669 |
| HETATM | 2670 | O | HOH | S | 235 | 55.405 | 91.022 | 139.852 | 1.00 | 0.00 | xxxx | 2670 |
| HETATM | 2671 | O | HOH | S | 236 | 40.342 | 65.511 | 164.886 | 1.00 | 0.00 | xxxx | 2671 |
| HETATM | 2672 | O | HOH | S | 237 | 66.483 | 99.541 | 155.727 | 1.00 | 0.00 | xxxx | 2672 |
| HETATM | 2673 | O | HOH | S | 238 | 69.457 | 86.310 | 171.372 | 1.00 | 0.00 | xxxx | 2673 |
| HETATM | 2674 | O | HOH | S | 239 | 44.207 | 49.050 | 155.766 | 1.00 | 0.00 | xxxx | 2674 |
| HETATM | 2675 | O | HOH | S | 240 | 77.984 | 88.107 | 149.281 | 1.00 | 0.00 | xxxx | 2675 |
| HETATM | 2676 | O | HOH | S | 241 | 63.936 | 65.431 | 143.959 | 1.00 | 0.00 | xxxx | 2676 |
| HETATM | 2677 | O | HOH | S | 242 | 59.342 | 56.830 | 138.270 | 1.00 | 0.00 | xxxx | 2677 |
| HETATM | 2678 | O | HOH | S | 243 | 57.960 | 56.896 | 136.242 | 1.00 | 0.00 | xxxx | 2678 |
| HETATM | 2679 | O | HOH | S | 244 | 58.013 | 47.012 | 148.836 | 1.00 | 0.00 | xxxx | 2679 |
| HETATM | 2680 | O | HOH | S | 245 | 75.427 | 69.810 | 142.388 | 1.00 | 0.00 | xxxx | 2680 |
| HETATM | 2681 | O | HOH | S | 246 | 73.826 | 68.223 | 143.334 | 1.00 | 0.00 | xxxx | 2681 |
| HETATM | 2682 | O | HOH | S | 247 | 42.971 | 82.759 | 155.019 | 1.00 | 0.00 | xxxx | 2682 |
| HETATM | 2683 | O | HOH | S | 248 | 52.571 | 85.235 | 164.090 | 1.00 | 0.00 | xxxx | 2683 |
| HETATM | 2684 | O | HOH | S | 249 | 45.214 | 46.605 | 152.420 | 1.00 | 0.00 | xxxx | 2684 |
| HETATM | 2685 | O | HOH | S | 250 | 29.343 | 67.140 | 135.830 | 1.00 | 0.00 | xxxx | 2685 |
| HETATM | 2686 | O | HOH | S | 251 | 32.366 | 55.742 | 159.321 | 1.00 | 0.00 | xxxx | 2686 |
| HETATM | 2687 | O | HOH | S | 252 | 24.677 | 53.488 | 148.443 | 1.00 | 0.00 | xxxx | 2687 |
| HETATM | 2688 | O | HOH | S | 253 | 53.247 | 94.555 | 156.841 | 1.00 | 0.00 | xxxx | 2688 |
| HETATM | 2689 | O | HOH | S | 254 | 74.994 | 84.859 | 143.239 | 1.00 | 0.00 | xxxx | 2689 |
| HETATM | 2690 | O | HOH | S | 255 | 42.936 | 91.602 | 150.830 | 1.00 | 0.00 | xxxx | 2690 |
| HETATM | 2691 | O | HOH | S | 256 | 58.422 | 83.991 | 169.629 | 1.00 | 0.00 | xxxx | 2691 |
| HETATM | 2692 | O | HOH | S | 257 | 69.838 | 92.467 | 158.698 | 1.00 | 0.00 | xxxx | 2692 |
| HETATM | 2693 | O | HOH | S | 258 | 68.045 | 85.333 | 139.314 | 1.00 | 0.00 | xxxx | 2693 |
| HETATM | 2694 | O | HOH | S | 259 | 60.263 | 52.077 | 139.361 | 1.00 | 0.00 | xxxx | 2694 |
| HETATM | 2695 | O | HOH | S | 260 | 27.165 | 65.538 | 140.881 | 1.00 | 0.00 | xxxx | 2695 |
| HETATM | 2696 | O | HOH | S | 261 | 64.093 | 93.917 | 162.579 | 1.00 | 0.00 | xxxx | 2696 |
| HETATM | 2697 | O | HOH | S | 262 | 58.261 | 93.702 | 145.184 | 1.00 | 0.00 | xxxx | 2697 |
| HETATM | 2698 | O | HOH | S | 263 | 43.274 | 41.321 | 143.716 | 1.00 | 0.00 | xxxx | 2698 |
| HETATM | 2699 | O | HOH | S | 264 | 44.457 | 63.916 | 165.291 | 1.00 | 0.00 | xxxx | 2699 |
| HETATM | 2700 | O | HOH | S | 265 | 42.371 | 41.840 | 141.713 | 1.00 | 0.00 | xxxx | 2700 |
| HETATM | 2701 | O | HOH | S | 266 | 32.912 | 69.796 | 134.996 | 1.00 | 0.00 | xxxx | 2701 |
| HETATM | 2702 | O | HOH | S | 267 | 38.574 | 62.379 | 129.054 | 1.00 | 0.00 | xxxx | 2702 |
| HETATM | 2703 | O | HOH | S | 268 | 52.919 | 55.185 | 167.684 | 1.00 | 0.00 | xxxx | 2703 |
| HETATM | 2704 | O | HOH | S | 269 | 42.297 | 73.045 | 138.008 | 1.00 | 0.00 | xxxx | 2704 |
| HETATM | 2705 | O | HOH | S | 270 | 55.678 | 97.395 | 147.147 | 1.00 | 0.00 | xxxx | 2705 |
| HETATM | 2706 | O | HOH | S | 271 | 52.073 | 77.661 | 142.443 | 1.00 | 0.00 | xxxx | 2706 |
| HETATM | 2707 | O | HOH | S | 272 | 53.656 | 79.318 | 140.562 | 1.00 | 0.00 | xxxx | 2707 |
| HETATM | 2708 | O | HOH | S | 273 | 46.237 | 72.352 | 160.290 | 1.00 | 0.00 | xxxx | 2708 |
| HETATM | 2709 | O | HOH | S | 274 | 46.994 | 92.936 | 143.057 | 1.00 | 0.00 | xxxx | 2709 |
| HETATM | 2710 | O | HOH | S | 275 | 44.477 | 81.823 | 145.212 | 1.00 | 0.00 | xxxx | 2710 |
| HETATM | 2711 | O | HOH | S | 276 | 54.031 | 87.747 | 163.437 | 1.00 | 0.00 | xxxx | 2711 |
| HETATM | 2712 | O | HOH | S | 277 | 47.434 | 52.681 | 166.283 | 1.00 | 0.00 | xxxx | 2712 |
| HETATM | 2713 | O | HOH | S | 278 | 59.600 | 57.004 | 158.284 | 1.00 | 0.00 | xxxx | 2713 |
| HETATM | 2714 | O | HOH | S | 279 | 60.963 | 54.848 | 139.584 | 1.00 | 0.00 | xxxx | 2714 |
| HETATM | 2715 | O | HOH | S | 280 | 37.148 | 63.959 | 132.391 | 1.00 | 0.00 | xxxx | 2715 |
| HETATM | 2716 | O | HOH | S | 281 | 28.986 | 71.953 | 146.829 | 1.00 | 0.00 | xxxx | 2716 |
| HETATM | 2717 | O | HOH | S | 282 | 68.512 | 94.290 | 162.575 | 1.00 | 0.00 | xxxx | 2717 |
| HETATM | 2718 | O | HOH | S | 283 | 52.234 | 40.696 | 137.518 | 1.00 | 0.00 | xxxx | 2718 |
| HETATM | 2719 | O | HOH | S | 284 | 66.206 | 85.004 | 174.135 | 1.00 | 0.00 | xxxx | 2719 |
| HETATM | 2720 | O | HOH | S | 285 | 75.905 | 76.142 | 152.137 | 1.00 | 0.00 | xxxx | 2720 |
| HETATM | 2721 | O | HOH | S | 286 | 50.731 | 54.152 | 132.775 | 1.00 | 0.00 | xxxx | 2721 |
| HETATM | 2722 | O | HOH | S | 287 | 54.504 | 71.995 | 168.294 | 1.00 | 0.00 | xxxx | 2722 |
| HETATM | 2723 | O | HOH | S | 288 | 56.248 | 96.171 | 141.897 | 1.00 | 0.00 | xxxx | 2723 |
| HETATM | 2724 | O | HOH | S | 289 | 69.486 | 62.152 | 150.746 | 1.00 | 0.00 | xxxx | 2724 |
| HETATM | 2725 | O | HOH | S | 290 | 40.024 | 40.276 | 140.882 | 1.00 | 0.00 | xxxx | 2725 |
| HETATM | 2726 | O | HOH | S | 291 | 44.385 | 41.941 | 132.405 | 1.00 | 0.00 | xxxx | 2726 |
| HETATM | 2727 | O | HOH | S | 292 | 71.883 | 78.785 | 169.503 | 1.00 | 0.00 | xxxx | 2727 |
| HETATM | 2728 | O | HOH | S | 293 | 56.087 | 94.966 | 139.562 | 1.00 | 0.00 | xxxx | 2728 |
| HETATM | 2729 | O | HOH | S | 294 | 50.604 | 86.542 | 161.142 | 1.00 | 0.00 | xxxx | 2729 |
| HETATM | 2730 | O | HOH | S | 295 | 49.091 | 55.663 | 133.859 | 1.00 | 0.00 | xxxx | 2730 |
| HETATM | 2731 | O | HOH | S | 296 | 62.140 | 68.428 | 142.203 | 1.00 | 0.00 | xxxx | 2731 |
| HETATM | 2732 | O | HOH | S | 297 | 58.188 | 60.998 | 161.107 | 1.00 | 0.00 | xxxx | 2732 |
| HETATM | 2733 | O | HOH | S | 298 | 34.634 | 46.282 | 149.220 | 1.00 | 0.00 | xxxx | 2733 |
| HETATM | 2734 | O | HOH | S | 299 | 53.105 | 97.360 | 156.777 | 1.00 | 0.00 | xxxx | 2734 |
| HETATM | 2735 | O | HOH | S | 300 | 23.462 | 59.601 | 146.117 | 1.00 | 0.00 | xxxx | 2735 |
| HETATM | 2736 | O | HOH | S | 301 | 55.964 | 92.355 | 162.949 | 1.00 | 0.00 | xxxx | 2736 |
| HETATM | 2737 | O | HOH | S | 302 | 27.393 | 66.398 | 134.317 | 1.00 | 0.00 | xxxx | 2737 |
| HETATM | 2738 | O | HOH | S | 303 | 61.386 | 88.815 | 145.169 | 1.00 | 0.00 | xxxx | 2738 |
| HETATM | 2739 | O | HOH | S | 304 | 51.190 | 51.469 | 157.598 | 1.00 | 0.00 | xxxx | 2739 |
| HETATM | 2740 | O | HOH | S | 305 | 25.996 | 59.783 | 135.372 | 1.00 | 0.00 | xxxx | 2740 |
| HETATM | 2741 | O | HOH | S | 306 | 33.722 | 43.191 | 142.370 | 1.00 | 0.00 | xxxx | 2741 |
| HETATM | 2742 | O | HOH | S | 307 | 42.824 | 79.527 | 144.482 | 1.00 | 0.00 | xxxx | 2742 |
| HETATM | 2743 | O | HOH | S | 308 | 46.683 | 73.714 | 134.659 | 1.00 | 0.00 | xxxx | 2743 |
| HETATM | 2744 | O | HOH | S | 309 | 39.500 | 50.446 | 155.833 | 1.00 | 0.00 | xxxx | 2744 |
| HETATM | 2745 | O | HOH | S | 310 | 55.858 | 87.737 | 140.514 | 1.00 | 0.00 | xxxx | 2745 |
| HETATM | 2746 | O | HOH | S | 311 | 51.193 | 56.462 | 133.700 | 1.00 | 0.00 | xxxx | 2746 |
| HETATM | 2747 | O | HOH | S | 312 | 59.165 | 45.441 | 139.785 | 1.00 | 0.00 | xxxx | 2747 |
| HETATM | 2748 | O | HOH | S | 313 | 53.333 | 78.458 | 170.965 | 1.00 | 0.00 | xxxx | 2748 |
| HETATM | 2749 | O | HOH | S | 314 | 49.285 | 85.987 | 141.326 | 1.00 | 0.00 | xxxx | 2749 |
| HETATM | 2750 | O | HOH | S | 315 | 35.185 | 60.851 | 156.183 | 1.00 | 0.00 | xxxx | 2750 |
| HETATM | 2751 | O | HOH | S | 316 | 75.105 | 83.783 | 158.857 | 1.00 | 0.00 | xxxx | 2751 |
| HETATM | 2752 | O | HOH | S | 317 | 64.272 | 59.277 | 147.662 | 1.00 | 0.00 | xxxx | 2752 |
| HETATM | 2753 | O | HOH | S | 318 | 77.413 | 76.276 | 140.638 | 1.00 | 0.00 | xxxx | 2753 |
| HETATM | 2754 | O | HOH | S | 319 | 76.605 | 82.256 | 158.647 | 1.00 | 0.00 | xxxx | 2754 |
| HETATM | 2755 | O | HOH | S | 320 | 29.536 | 56.236 | 132.756 | 1.00 | 0.00 | xxxx | 2755 |
| HETATM | 2756 | O | HOH | S | 321 | 78.091 | 77.379 | 142.155 | 1.00 | 0.00 | xxxx | 2756 |
| HETATM | 2757 | O | HOH | S | 322 | 59.988 | 69.316 | 150.305 | 1.00 | 0.00 | xxxx | 2757 |
| HETATM | 2758 | O | HOH | S | 323 | 52.661 | 46.359 | 135.892 | 1.00 | 0.00 | xxxx | 2758 |
| HETATM | 2759 | O | HOH | S | 324 | 34.321 | 47.279 | 146.428 | 1.00 | 0.00 | xxxx | 2759 |
| HETATM | 2760 | O | HOH | S | 325 | 75.579 | 93.207 | 168.490 | 1.00 | 0.00 | xxxx | 2760 |
| HETATM | 2761 | O | HOH | S | 326 | 43.288 | 63.060 | 132.086 | 1.00 | 0.00 | xxxx | 2761 |
| HETATM | 2762 | O | HOH | S | 327 | 66.171 | 97.517 | 150.411 | 1.00 | 0.00 | xxxx | 2762 |
| HETATM | 2763 | O | HOH | S | 328 | 31.253 | 56.201 | 131.353 | 1.00 | 0.00 | xxxx | 2763 |
| HETATM | 2764 | O | HOH | S | 329 | 45.863 | 41.542 | 134.212 | 1.00 | 0.00 | xxxx | 2764 |
| HETATM | 2765 | O | HOH | S | 330 | 51.530 | 72.106 | 164.595 | 1.00 | 0.00 | xxxx | 2765 |
| HETATM | 2766 | O | HOH | S | 331 | 57.118 | 92.826 | 158.098 | 1.00 | 0.00 | xxxx | 2766 |
| HETATM | 2767 | O | HOH | S | 332 | 60.186 | 54.769 | 150.237 | 1.00 | 0.00 | xxxx | 2767 |
| HETATM | 2768 | O | HOH | S | 333 | 77.624 | 72.381 | 139.249 | 1.00 | 0.00 | xxxx | 2768 |
| HETATM | 2769 | O | HOH | S | 334 | 45.538 | 77.271 | 164.125 | 1.00 | 0.00 | xxxx | 2769 |
| HETATM | 2770 | O | HOH | S | 335 | 25.140 | 65.377 | 135.730 | 1.00 | 0.00 | xxxx | 2770 |
| HETATM | 2771 | O | HOH | S | 336 | 74.824 | 77.536 | 168.183 | 1.00 | 0.00 | xxxx | 2771 |
| HETATM | 2772 | O | HOH | S | 337 | 78.522 | 85.266 | 151.265 | 1.00 | 0.00 | xxxx | 2772 |
| HETATM | 2773 | O | HOH | S | 338 | 57.875 | 71.649 | 166.102 | 1.00 | 0.00 | xxxx | 2773 |
| HETATM | 2774 | O | HOH | S | 339 | 39.030 | 37.543 | 149.135 | 1.00 | 0.00 | xxxx | 2774 |
| HETATM | 2775 | O | HOH | S | 340 | 41.320 | 40.877 | 138.844 | 1.00 | 0.00 | xxxx | 2775 |
| HETATM | 2776 | O | HOH | S | 341 | 65.399 | 60.174 | 155.252 | 1.00 | 0.00 | xxxx | 2776 |
| HETATM | 2777 | O | HOH | S | 342 | 72.011 | 89.306 | 162.040 | 1.00 | 0.00 | xxxx | 2777 |
| HETATM | 2778 | O | HOH | S | 343 | 47.174 | 48.497 | 131.385 | 1.00 | 0.00 | xxxx | 2778 |
| HETATM | 2779 | O | HOH | S | 344 | 75.680 | 73.049 | 169.298 | 1.00 | 0.00 | xxxx | 2779 |
| HETATM | 2780 | O | HOH | S | 345 | 40.786 | 70.442 | 133.027 | 1.00 | 0.00 | xxxx | 2780 |
| HETATM | 2781 | O | HOH | S | 346 | 37.925 | 40.317 | 149.883 | 1.00 | 0.00 | xxxx | 2781 |
| HETATM | 2782 | O | HOH | S | 347 | 76.697 | 72.821 | 154.946 | 1.00 | 0.00 | xxxx | 2782 |
| HETATM | 2783 | O | HOH | S | 348 | 63.421 | 65.807 | 141.012 | 1.00 | 0.00 | xxxx | 2783 |
| HETATM | 2784 | O | HOH | S | 349 | 51.122 | 59.252 | 136.857 | 1.00 | 0.00 | xxxx | 2784 |
| HETATM | 2785 | O | HOH | S | 350 | 70.369 | 94.016 | 156.050 | 1.00 | 0.00 | xxxx | 2785 |
| HETATM | 2786 | O | HOH | S | 351 | 49.992 | 60.017 | 134.717 | 1.00 | 0.00 | xxxx | 2786 |
| HETATM | 2787 | O | HOH | S | 352 | 75.350 | 86.671 | 157.529 | 1.00 | 0.00 | xxxx | 2787 |
| HETATM | 2788 | O | HOH | S | 353 | 77.823 | 75.935 | 166.556 | 1.00 | 0.00 | xxxx | 2788 |
| HETATM | 2789 | O | HOH | S | 354 | 55.100 | 90.751 | 160.307 | 1.00 | 0.00 | xxxx | 2789 |
| HETATM | 2790 | O | HOH | S | 355 | 27.904 | 71.090 | 145.335 | 1.00 | 0.00 | xxxx | 2790 |
| HETATM | 2791 | O | HOH | S | 356 | 43.946 | 74.064 | 159.815 | 1.00 | 0.00 | xxxx | 2791 |
| HETATM | 2792 | O | HOH | S | 357 | 53.394 | 90.181 | 160.815 | 1.00 | 0.00 | xxxx | 2792 |
| HETATM | 2793 | O | HOH | S | 358 | 64.402 | 87.495 | 170.323 | 1.00 | 0.00 | xxxx | 2793 |
| HETATM | 2794 | O | HOH | S | 359 | 73.875 | 86.615 | 159.747 | 1.00 | 0.00 | xxxx | 2794 |
| HETATM | 2795 | O | HOH | S | 360 | 39.522 | 67.049 | 162.161 | 1.00 | 0.00 | xxxx | 2795 |
| HETATM | 2796 | O | HOH | S | 361 | 44.455 | 81.429 | 142.143 | 1.00 | 0.00 | xxxx | 2796 |
| HETATM | 2797 | O | HOH | S | 362 | 25.454 | 62.332 | 138.307 | 1.00 | 0.00 | xxxx | 2797 |
| HETATM | 2798 | O | HOH | S | 363 | 55.641 | 89.241 | 165.418 | 1.00 | 0.00 | xxxx | 2798 |
| HETATM | 2799 | O | HOH | S | 364 | 44.808 | 82.801 | 150.463 | 1.00 | 0.00 | xxxx | 2799 |
| HETATM | 2800 | O | HOH | S | 365 | 72.236 | 81.203 | 173.001 | 1.00 | 0.00 | xxxx | 2800 |
| HETATM | 2801 | O | HOH | S | 366 | 68.892 | 66.917 | 168.696 | 1.00 | 0.00 | xxxx | 2801 |
| HETATM | 2802 | O | HOH | S | 367 | 56.464 | 50.291 | 134.278 | 1.00 | 0.00 | xxxx | 2802 |
| HETATM | 2803 | O | HOH | S | 368 | 62.491 | 61.198 | 139.575 | 1.00 | 0.00 | xxxx | 2803 |
| HETATM | 2804 | O | HOH | S | 369 | 60.971 | 62.929 | 137.733 | 1.00 | 0.00 | xxxx | 2804 |
| HETATM | 2805 | O | HOH | S | 370 | 61.950 | 98.032 | 148.878 | 1.00 | 0.00 | xxxx | 2805 |
| HETATM | 2806 | O | HOH | S | 371 | 30.702 | 69.382 | 135.319 | 1.00 | 0.00 | xxxx | 2806 |
| HETATM | 2807 | O | HOH | S | 372 | 72.835 | 83.672 | 172.687 | 1.00 | 0.00 | xxxx | 2807 |
| HETATM | 2808 | O | HOH | S | 373 | 30.631 | 53.789 | 158.886 | 1.00 | 0.00 | xxxx | 2808 |
| HETATM | 2809 | O | HOH | S | 374 | 58.769 | 92.752 | 159.679 | 1.00 | 0.00 | xxxx | 2809 |
| HETATM | 2810 | O | HOH | S | 375 | 55.480 | 50.180 | 157.300 | 1.00 | 0.00 | xxxx | 2810 |
| HETATM | 2811 | O | HOH | S | 376 | 39.942 | 68.852 | 162.294 | 1.00 | 0.00 | xxxx | 2811 |
| HETATM | 2812 | O | HOH | S | 377 | 63.692 | 63.207 | 141.734 | 1.00 | 0.00 | xxxx | 2812 |
| HETATM | 2813 | O | HOH | S | 378 | 56.000 | 76.808 | 172.839 | 1.00 | 0.00 | xxxx | 2813 |
| HETATM | 2814 | O | HOH | S | 379 | 74.372 | 83.991 | 169.863 | 1.00 | 0.00 | xxxx | 2814 |
| HETATM | 2815 | O | HOH | S | 380 | 22.549 | 63.907 | 135.222 | 1.00 | 0.00 | xxxx | 2815 |
| HETATM | 2816 | O | HOH | S | 381 | 23.412 | 65.534 | 135.198 | 1.00 | 0.00 | xxxx | 2816 |
| HETATM | 2817 | O | HOH | S | 382 | 60.263 | 67.901 | 140.518 | 1.00 | 0.00 | xxxx | 2817 |
| HETATM | 2818 | O | HOH | S | 383 | 45.221 | 69.427 | 160.951 | 1.00 | 0.00 | xxxx | 2818 |
| HETATM | 2819 | O | HOH | S | 384 | 66.498 | 92.424 | 164.876 | 1.00 | 0.00 | xxxx | 2819 |
| HETATM | 2820 | O | HOH | S | 385 | 37.032 | 71.203 | 135.720 | 1.00 | 0.00 | xxxx | 2820 |
| HETATM | 2821 | O | HOH | S | 386 | 77.411 | 79.795 | 146.037 | 1.00 | 0.00 | xxxx | 2821 |
| HETATM | 2822 | O | HOH | S | 387 | 47.947 | 41.913 | 141.390 | 1.00 | 0.00 | xxxx | 2822 |
| HETATM | 2823 | O | HOH | S | 388 | 60.964 | 60.212 | 161.343 | 1.00 | 0.00 | xxxx | 2823 |
| HETATM | 2824 | O | HOH | S | 389 | 45.817 | 86.091 | 143.837 | 1.00 | 0.00 | xxxx | 2824 |
| HETATM | 2825 | O | HOH | S | 390 | 76.619 | 77.018 | 167.654 | 1.00 | 0.00 | xxxx | 2825 |
| HETATM | 2826 | O | HOH | S | 391 | 42.858 | 79.646 | 148.541 | 1.00 | 0.00 | xxxx | 2826 |
| HETATM | 2827 | O | HOH | S | 392 | 54.410 | 83.651 | 164.891 | 1.00 | 0.00 | xxxx | 2827 |
| HETATM | 2828 | O | HOH | S | 393 | 70.894 | 63.870 | 144.402 | 1.00 | 0.00 | xxxx | 2828 |
| HETATM | 2829 | O | HOH | S | 394 | 36.961 | 58.639 | 165.920 | 1.00 | 0.00 | xxxx | 2829 |
| HETATM | 2830 | O | HOH | S | 395 | 58.708 | 92.155 | 141.294 | 1.00 | 0.00 | xxxx | 2830 |
| HETATM | 2831 | O | HOH | S | 396 | 78.814 | 78.170 | 154.417 | 1.00 | 0.00 | xxxx | 2831 |
| HETATM | 2832 | O | HOH | S | 397 | 42.611 | 75.099 | 155.809 | 1.00 | 0.00 | xxxx | 2832 |
| HETATM | 2833 | O | HOH | S | 398 | 72.716 | 86.788 | 143.422 | 1.00 | 0.00 | xxxx | 2833 |
| HETATM | 2834 | O | HOH | S | 399 | 43.847 | 80.375 | 147.143 | 1.00 | 0.00 | xxxx | 2834 |
| HETATM | 2835 | O | HOH | S | 400 | 44.815 | 39.074 | 135.864 | 1.00 | 0.00 | xxxx | 2835 |
| HETATM | 2836 | O | HOH | S | 401 | 62.460 | 74.450 | 172.476 | 1.00 | 0.00 | xxxx | 2836 |
| HETATM | 2837 | O | HOH | S | 402 | 79.224 | 81.985 | 160.103 | 1.00 | 0.00 | xxxx | 2837 |
| HETATM | 2838 | O | HOH | S | 403 | 79.319 | 80.456 | 154.759 | 1.00 | 0.00 | xxxx | 2838 |

### Example 12. Crystal Structure Coordinates for a T. thermosaccharolyticum Glucose-Galactose Binding Protein: TTHGBP182C⋅Acrylodan (Acrylodan Attached to W182C Mutant+R91K, Q18E Double Mutant)

Naming is standard three-letter amino acid code.
For hetero atom (HETATM) records:

CA, calcium
HOH, water
ACR, Acrylodan
K, potassium
EDO, ethylene glycol

| | | | | | | | | | | | | |
|---|---|---|---|---|---|---|---|---|---|---|---|---|
| ATOM | 1 | O | MET | A | 1 | 45.210 | 37.591 | 106.914 | 1.00 | 0.00 | xxxx | 1 |
| ATOM | 2 | N | MET | A | 1 | 45.762 | 36.296 | 109.277 | 1.00 | 0.00 | xxxx | 2 |
| ATOM | 3 | CA | MET | A | 1 | 46.329 | 35.790 | 108.035 | 1.00 | 0.00 | xxxx | 3 |
| ATOM | 4 | C | MET | A | 1 | 46.138 | 36.788 | 106.900 | 1.00 | 0.00 | xxxx | 4 |
| ATOM | 5 | CB | MET | A | 1 | 45.670 | 34.462 | 107.649 | 1.00 | 0.00 | xxxx | 5 |
| ATOM | 6 | CG | MET | A | 1 | 45.720 | 33.401 | 108.732 | 1.00 | 0.00 | xxxx | 6 |
| ATOM | 7 | SD | MET | A | 1 | 47.393 | 32.806 | 109.049 | 1.00 | 0.00 | xxxx | 7 |
| ATOM | 8 | CE | MET | A | 1 | 47.362 | 32.657 | 110.834 | 1.00 | 0.00 | xxxx | 8 |
| ATOM | 9 | N | LYS | A | 2 | 47.025 | 36.728 | 105.914 | 1.00 | 0.00 | xxxx | 9 |
| ATOM | 10 | CA | LYS | A | 2 | 46.833 | 37.493 | 104.692 | 1.00 | 0.00 | xxxx | 10 |
| ATOM | 11 | C | LYS | A | 2 | 45.651 | 36.919 | 103.923 | 1.00 | 0.00 | xxxx | 11 |
| ATOM | 12 | O | LYS | A | 2 | 45.477 | 35.699 | 103.844 | 1.00 | 0.00 | xxxx | 12 |
| ATOM | 13 | CB | LYS | A | 2 | 48.101 | 37.412 | 103.835 | 1.00 | 0.00 | xxxx | 13 |
| ATOM | 14 | CG | LYS | A | 2 | 48.098 | 38.337 | 102.634 | 1.00 | 0.00 | xxxx | 14 |
| ATOM | 15 | CD | LYS | A | 2 | 49.485 | 38.473 | 102.012 | 1.00 | 0.00 | xxxx | 15 |
| ATOM | 16 | CE | LYS | A | 2 | 49.883 | 37.224 | 101.251 | 1.00 | 0.00 | xxxx | 16 |
| ATOM | 17 | NZ | LYS | A | 2 | 51.165 | 37.424 | 100.517 | 1.00 | 0.00 | xxxx | 17 |
| ATOM | 18 | N | GLN | A | 3 | 44.819 | 37.797 | 103.364 | 1.00 | 0.00 | xxxx | 18 |
| ATOM | 19 | CA | GLN | A | 3 | 43.674 | 37.341 | 102.583 | 1.00 | 0.00 | xxxx | 19 |
| ATOM | 20 | C | GLN | A | 3 | 44.115 | 37.047 | 101.155 | 1.00 | 0.00 | xxxx | 20 |
| ATOM | 21 | O | GLN | A | 3 | 44.627 | 37.928 | 100.458 | 1.00 | 0.00 | xxxx | 21 |
| ATOM | 22 | CB | GLN | A | 3 | 42.546 | 38.370 | 102.593 | 1.00 | 0.00 | xxxx | 22 |
| ATOM | 23 | CG | GLN | A | 3 | 41.288 | 37.854 | 101.877 | 1.00 | 0.00 | xxxx | 23 |
| ATOM | 24 | CD | GLN | A | 3 | 40.152 | 38.869 | 101.821 | 1.00 | 0.00 | xxxx | 24 |
| ATOM | 25 | OE1 | GLN | A | 3 | 40.188 | 39.812 | 101.033 | 1.00 | 0.00 | xxxx | 25 |
| ATOM | 26 | NE2 | GLN | A | 3 | 39.133 | 38.666 | 102.650 | 1.00 | 0.00 | xxxx | 26 |
| ATOM | 27 | N | LEU | A | 4 | 43.922 | 35.808 | 100.729 | 1.00 | 0.00 | xxxx | 27 |
| ATOM | 28 | CA | LEU | A | 4 | 44.155 | 35.405 | 99.356 | 1.00 | 0.00 | xxxx | 28 |
| ATOM | 29 | C | LEU | A | 4 | 42.819 | 35.406 | 98.634 | 1.00 | 0.00 | xxxx | 29 |
| ATOM | 30 | O | LEU | A | 4 | 41.807 | 34.955 | 99.179 | 1.00 | 0.00 | xxxx | 30 |
| ATOM | 31 | CB | LEU | A | 4 | 44.758 | 33.999 | 99.317 | 1.00 | 0.00 | xxxx | 31 |
| ATOM | 32 | CG | LEU | A | 4 | 46.128 | 33.906 | 99.989 | 1.00 | 0.00 | xxxx | 32 |
| ATOM | 33 | CD1 | LEU | A | 4 | 46.603 | 32.466 | 100.045 | 1.00 | 0.00 | xxxx | 33 |
| ATOM | 34 | CD2 | LEU | A | 4 | 47.122 | 34.771 | 99.240 | 1.00 | 0.00 | xxxx | 34 |
| ATOM | 35 | N | ASN | A | 5 | 42.816 | 35.928 | 97.417 | 1.00 | 0.00 | xxxx | 35 |
| ATOM | 36 | CA | ASN | A | 5 | 41.601 | 36.070 | 96.627 | 1.00 | 0.00 | xxxx | 36 |
| ATOM | 37 | C | ASN | A | 5 | 41.719 | 35.224 | 95.378 | 1.00 | 0.00 | xxxx | 37 |
| ATOM | 38 | O | ASN | A | 5 | 42.670 | 35.378 | 94.602 | 1.00 | 0.00 | xxxx | 38 |
| ATOM | 39 | CB | ASN | A | 5 | 41.379 | 37.526 | 96.236 | 1.00 | 0.00 | xxxx | 39 |
| ATOM | 40 | CG | ASN | A | 5 | 40.748 | 38.312 | 97.342 | 1.00 | 0.00 | xxxx | 40 |
| ATOM | 41 | OD1 | ASN | A | 5 | 39.555 | 38.182 | 97.600 | 1.00 | 0.00 | xxxx | 41 |
| ATOM | 42 | ND2 | ASN | A | 5 | 41.547 | 39.128 | 98.019 | 1.00 | 0.00 | xxxx | 42 |
| ATOM | 43 | N | ILE | A | 6 | 40.751 | 34.341 | 95.183 | 1.00 | 0.00 | xxxx | 43 |
| ATOM | 44 | CA | ILE | A | 6 | 40.687 | 33.487 | 94.005 | 1.00 | 0.00 | xxxx | 44 |
| ATOM | 45 | C | ILE | A | 6 | 39.419 | 33.829 | 93.242 | 1.00 | 0.00 | xxxx | 45 |
| ATOM | 46 | O | ILE | A | 6 | 38.324 | 33.826 | 93.814 | 1.00 | 0.00 | xxxx | 46 |
| ATOM | 47 | CB | ILE | A | 6 | 40.716 | 31.997 | 94.388 | 1.00 | 0.00 | xxxx | 47 |
| ATOM | 48 | CG1 | ILE | A | 6 | 42.005 | 31.679 | 95.151 | 1.00 | 0.00 | xxxx | 48 |
| ATOM | 49 | CG2 | ILE | A | 6 | 40.600 | 31.126 | 93.145 | 1.00 | 0.00 | xxxx | 49 |
| ATOM | 50 | CD1 | ILE | A | 6 | 41.999 | 30.321 | 95.825 | 1.00 | 0.00 | xxxx | 50 |
| ATOM | 51 | N | GLY | A | 7 | 39.570 | 34.145 | 91.963 | 1.00 | 0.00 | xxxx | 51 |
| ATOM | 52 | CA | GLY | A | 7 | 38.413 | 34.396 | 91.134 | 1.00 | 0.00 | xxxx | 52 |
| ATOM | 53 | C | GLY | A | 7 | 37.924 | 33.090 | 90.543 | 1.00 | 0.00 | xxxx | 53 |
| ATOM | 54 | O | GLY | A | 7 | 38.686 | 32.412 | 89.855 | 1.00 | 0.00 | xxxx | 54 |
| ATOM | 55 | N | VAL | A | 8 | 36.679 | 32.708 | 90.822 | 1.00 | 0.00 | xxxx | 55 |
| ATOM | 56 | CA | VAL | A | 8 | 36.120 | 31.439 | 90.353 | 1.00 | 0.00 | xxxx | 56 |
| ATOM | 57 | C | VAL | A | 8 | 34.927 | 31.749 | 89.466 | 1.00 | 0.00 | xxxx | 57 |
| ATOM | 58 | O | VAL | A | 8 | 33.987 | 32.428 | 89.901 | 1.00 | 0.00 | xxxx | 58 |
| ATOM | 59 | CB | VAL | A | 8 | 35.678 | 30.544 | 91.522 | 1.00 | 0.00 | xxxx | 59 |
| ATOM | 60 | CG1 | VAL | A | 8 | 35.179 | 29.191 | 90.997 | 1.00 | 0.00 | xxxx | 60 |
| ATOM | 61 | CG2 | VAL | A | 8 | 36.802 | 30.361 | 92.528 | 1.00 | 0.00 | xxxx | 61 |
| ATOM | 62 | N | ALA | A | 9 | 34.956 | 31.258 | 88.235 | 1.00 | 0.00 | xxxx | 62 |
| ATOM | 63 | CA | ALA | A | 9 | 33.838 | 31.406 | 87.312 | 1.00 | 0.00 | xxxx | 63 |
| ATOM | 64 | C | ALA | A | 9 | 33.262 | 30.028 | 87.040 | 1.00 | 0.00 | xxxx | 64 |
| ATOM | 65 | O | ALA | A | 9 | 33.993 | 29.131 | 86.602 | 1.00 | 0.00 | xxxx | 65 |
| ATOM | 66 | CB | ALA | A | 9 | 34.284 | 32.061 | 86.002 | 1.00 | 0.00 | xxxx | 66 |
| ATOM | 67 | N | ILE | A | 10 | 31.967 | 29.858 | 87.321 | 1.00 | 0.00 | xxxx | 67 |
| ATOM | 68 | CA | ILE | A | 10 | 31.236 | 28.619 | 87.049 | 1.00 | 0.00 | xxxx | 68 |
| ATOM | 69 | C | ILE | A | 10 | 30.422 | 28.837 | 85.784 | 1.00 | 0.00 | xxxx | 69 |
| ATOM | 70 | O | ILE | A | 10 | 29.765 | 29.873 | 85.640 | 1.00 | 0.00 | xxxx | 70 |
| ATOM | 71 | CB | ILE | A | 10 | 30.325 | 28.257 | 88.238 | 1.00 | 0.00 | xxxx | 71 |
| ATOM | 72 | CG1 | ILE | A | 10 | 31.138 | 28.181 | 89.538 | 1.00 | 0.00 | xxxx | 72 |
| ATOM | 73 | CG2 | ILE | A | 10 | 29.577 | 26.954 | 87.986 | 1.00 | 0.00 | xxxx | 73 |
| ATOM | 74 | CD1 | ILE | A | 10 | 32.132 | 27.050 | 89.563 | 1.00 | 0.00 | xxxx | 74 |
| ATOM | 75 | N | TYR | A | 11 | 30.445 | 27.866 | 84.863 | 1.00 | 0.00 | xxxx | 75 |
| ATOM | 76 | CA | TYR | A | 11 | 29.862 | 28.130 | 83.545 | 1.00 | 0.00 | xxxx | 76 |
| ATOM | 77 | C | TYR | A | 11 | 28.373 | 28.468 | 83.643 | 1.00 | 0.00 | xxxx | 77 |
| ATOM | 78 | O | TYR | A | 11 | 27.889 | 29.339 | 82.916 | 1.00 | 0.00 | xxxx | 78 |
| ATOM | 79 | CB | TYR | A | 11 | 30.170 | 27.001 | 82.546 | 1.00 | 0.00 | xxxx | 79 |
| ATOM | 80 | CG | TYR | A | 11 | 29.092 | 25.948 | 82.396 | 1.00 | 0.00 | xxxx | 80 |
| ATOM | 81 | CD1 | TYR | A | 11 | 28.144 | 26.043 | 81.384 | 1.00 | 0.00 | xxxx | 81 |
| ATOM | 82 | CD2 | TYR | A | 11 | 29.034 | 24.856 | 83.261 | 1.00 | 0.00 | xxxx | 82 |
| ATOM | 83 | CE1 | TYR | A | 11 | 27.158 | 25.080 | 81.241 | 1.00 | 0.00 | xxxx | 83 |
| ATOM | 84 | CE2 | TYR | A | 11 | 28.045 | 23.892 | 83.125 | 1.00 | 0.00 | xxxx | 84 |
| ATOM | 85 | CZ | TYR | A | 11 | 27.118 | 24.018 | 82.115 | 1.00 | 0.00 | xxxx | 85 |
| ATOM | 86 | OH | TYR | A | 11 | 26.134 | 23.080 | 81.984 | 1.00 | 0.00 | xxxx | 86 |
| ATOM | 87 | N | LYS | A | 12 | 27.630 | 27.782 | 84.516 | 1.00 | 0.00 | xxxx | 87 |
| ATOM | 88 | CA | LYS | A | 12 | 26.283 | 28.208 | 84.866 | 1.00 | 0.00 | xxxx | 88 |
| ATOM | 89 | C | LYS | A | 12 | 25.909 | 27.598 | 86.210 | 1.00 | 0.00 | xxxx | 89 |
| ATOM | 90 | O | LYS | A | 12 | 26.226 | 26.436 | 86.496 | 1.00 | 0.00 | xxxx | 90 |
| ATOM | 91 | CB | LYS | A | 12 | 25.243 | 27.891 | 83.780 | 1.00 | 0.00 | xxxx | 91 |
| ATOM | 92 | CG | LYS | A | 12 | 24.862 | 26.446 | 83.644 | 1.00 | 0.00 | xxxx | 92 |
| ATOM | 93 | CD | LYS | A | 12 | 23.817 | 26.256 | 82.538 | 1.00 | 0.00 | xxxx | 93 |
| ATOM | 94 | CE | LYS | A | 12 | 23.394 | 24.805 | 82.448 | 1.00 | 0.00 | xxxx | 94 |
| ATOM | 95 | NZ | LYS | A | 12 | 22.368 | 24.593 | 81.393 | 1.00 | 0.00 | xxxx | 95 |
| ATOM | 96 | N | PHE | A | 13 | 25.249 | 28.403 | 87.036 | 1.00 | 0.00 | xxxx | 96 |
| ATOM | 97 | CA | PHE | A | 13 | 24.853 | 27.945 | 88.361 | 1.00 | 0.00 | xxxx | 97 |
| ATOM | 98 | C | PHE | A | 13 | 23.757 | 26.882 | 88.320 | 1.00 | 0.00 | xxxx | 98 |
| ATOM | 99 | O | PHE | A | 13 | 23.655 | 26.093 | 89.267 | 1.00 | 0.00 | xxxx | 99 |
| ATOM | 100 | CB | PHE | A | 13 | 24.403 | 29.131 | 89.226 | 1.00 | 0.00 | xxxx | 100 |
| ATOM | 101 | CG | PHE | A | 13 | 25.524 | 29.827 | 89.958 | 1.00 | 0.00 | xxxx | 101 |
| ATOM | 102 | CD1 | PHE | A | 13 | 26.849 | 29.623 | 89.627 | 1.00 | 0.00 | xxxx | 102 |
| ATOM | 103 | CD2 | PHE | A | 13 | 25.234 | 30.700 | 90.997 | 1.00 | 0.00 | xxxx | 103 |
| ATOM | 104 | CE1 | PHE | A | 13 | 27.871 | 30.277 | 90.315 | 1.00 | 0.00 | xxxx | 104 |
| ATOM | 105 | CE2 | PHE | A | 13 | 26.245 | 31.352 | 91.691 | 1.00 | 0.00 | xxxx | 105 |
| ATOM | 106 | CZ | PHE | A | 13 | 27.567 | 31.139 | 91.351 | 1.00 | 0.00 | xxxx | 106 |
| ATOM | 107 | N | ASP | A | 14 | 22.962 | 26.805 | 87.251 | 1.00 | 0.00 | xxxx | 107 |
| ATOM | 108 | CA | ASP | A | 14 | 21.868 | 25.834 | 87.224 | 1.00 | 0.00 | xxxx | 108 |
| ATOM | 109 | C | ASP | A | 14 | 22.293 | 24.430 | 86.781 | 1.00 | 0.00 | xxxx | 109 |
| ATOM | 110 | O | ASP | A | 14 | 21.433 | 23.546 | 86.663 | 1.00 | 0.00 | xxxx | 110 |
| ATOM | 111 | CB | ASP | A | 14 | 20.671 | 26.348 | 86.413 | 1.00 | 0.00 | xxxx | 111 |
| ATOM | 112 | CG | ASP | A | 14 | 21.055 | 26.809 | 85.025 | 1.00 | 0.00 | xxxx | 112 |
| ATOM | 113 | OD1 | ASP | A | 14 | 22.089 | 27.496 | 84.889 | 1.00 | 0.00 | xxxx | 113 |
| ATOM | 114 | OD2 | ASP | A | 14 | 20.313 | 26.493 | 84.071 | 1.00 | 0.00 | xxxx | 114 |
| ATOM | 115 | N | ASP | A | 15 | 23.582 | 24.188 | 86.573 | 1.00 | 0.00 | xxxx | 115 |
| ATOM | 116 | CA | ASP | A | 15 | 24.059 | 22.858 | 86.222 | 1.00 | 0.00 | xxxx | 116 |
| ATOM | 117 | C | ASP | A | 15 | 24.132 | 22.010 | 87.491 | 1.00 | 0.00 | xxxx | 117 |
| ATOM | 118 | O | ASP | A | 15 | 24.761 | 22.414 | 88.478 | 1.00 | 0.00 | xxxx | 118 |
| ATOM | 119 | CB | ASP | A | 15 | 25.435 | 22.975 | 85.574 | 1.00 | 0.00 | xxxx | 119 |
| ATOM | 120 | CG | ASP | A | 15 | 25.957 | 21.651 | 85.075 | 1.00 | 0.00 | xxxx | 120 |
| ATOM | 121 | OD1 | ASP | A | 15 | 25.794 | 21.363 | 83.873 | 1.00 | 0.00 | xxxx | 121 |
| ATOM | 122 | OD2 | ASP | A | 15 | 26.530 | 20.893 | 85.881 | 1.00 | 0.00 | xxxx | 122 |
| ATOM | 123 | N | THR | A | 16 | 23.478 | 20.843 | 87.476 | 1.00 | 0.00 | xxxx | 123 |
| ATOM | 124 | CA | THR | A | 16 | 23.363 | 20.037 | 88.690 | 1.00 | 0.00 | xxxx | 124 |
| ATOM | 125 | C | THR | A | 16 | 24.724 | 19.543 | 89.166 | 1.00 | 0.00 | xxxx | 125 |
| ATOM | 126 | O | THR | A | 16 | 25.055 | 19.655 | 90.355 | 1.00 | 0.00 | xxxx | 126 |
| ATOM | 127 | CB | THR | A | 16 | 22.423 | 18.855 | 88.463 | 1.00 | 0.00 | xxxx | 127 |
| ATOM | 128 | OG1 | THR | A | 16 | 21.163 | 19.330 | 87.960 | 1.00 | 0.00 | xxxx | 128 |
| ATOM | 129 | CG2 | THR | A | 16 | 22.184 | 18.113 | 89.776 | 1.00 | 0.00 | xxxx | 129 |
| ATOM | 130 | N | PHE | A | 17 | 25.529 | 18.990 | 88.255 | 1.00 | 0.00 | xxxx | 130 |
| ATOM | 131 | CA | PHE | A | 17 | 26.844 | 18.493 | 88.656 | 1.00 | 0.00 | xxxx | 131 |
| ATOM | 132 | C | PHE | A | 17 | 27.726 | 19.624 | 89.167 | 1.00 | 0.00 | xxxx | 132 |
| ATOM | 133 | O | PHE | A | 17 | 28.395 | 19.494 | 90.206 | 1.00 | 0.00 | xxxx | 133 |
| ATOM | 134 | CB | PHE | A | 17 | 27.522 | 17.737 | 87.506 | 1.00 | 0.00 | xxxx | 134 |
| ATOM | 135 | CG | PHE | A | 17 | 28.944 | 17.377 | 87.815 | 1.00 | 0.00 | xxxx | 135 |
| ATOM | 136 | CD1 | PHE | A | 17 | 29.221 | 16.313 | 88.658 | 1.00 | 0.00 | xxxx | 136 |
| ATOM | 137 | CD2 | PHE | A | 17 | 29.992 | 18.137 | 87.324 | 1.00 | 0.00 | xxxx | 137 |
| ATOM | 138 | CE1 | PHE | A | 17 | 30.527 | 15.998 | 89.000 | 1.00 | 0.00 | xxxx | 138 |
| ATOM | 139 | CE2 | PHE | A | 17 | 31.315 | 17.815 | 87.651 | 1.00 | 0.00 | xxxx | 139 |
| ATOM | 140 | CZ | PHE | A | 17 | 31.572 | 16.752 | 88.491 | 1.00 | 0.00 | xxxx | 140 |
| ATOM | 141 | N | MET | A | 18 | 27.749 | 20.745 | 88.449 | 1.00 | 0.00 | xxxx | 141 |
| ATOM | 142 | CA | MET | A | 18 | 28.609 | 21.840 | 88.859 | 1.00 | 0.00 | xxxx | 142 |
| ATOM | 143 | C | MET | A | 18 | 28.149 | 22.496 | 90.158 | 1.00 | 0.00 | xxxx | 143 |
| ATOM | 144 | O | MET | A | 18 | 28.966 | 23.127 | 90.832 | 1.00 | 0.00 | xxxx | 144 |
| ATOM | 145 | CB | MET | A | 18 | 28.780 | 22.851 | 87.723 | 1.00 | 0.00 | xxxx | 145 |
| ATOM | 146 | CG | MET | A | 18 | 29.638 | 22.310 | 86.563 | 1.00 | 0.00 | xxxx | 146 |
| ATOM | 147 | SD | MET | A | 18 | 31.269 | 21.698 | 87.068 | 1.00 | 0.00 | xxxx | 147 |
| ATOM | 148 | CE | MET | A | 18 | 31.997 | 23.172 | 87.760 | 1.00 | 0.00 | xxxx | 148 |
| ATOM | 149 | N | THR | A | 19 | 26.872 | 22.368 | 90.531 | 1.00 | 0.00 | xxxx | 149 |
| ATOM | 150 | CA | THR | A | 19 | 26.451 | 22.829 | 91.850 | 1.00 | 0.00 | xxxx | 150 |
| ATOM | 151 | C | THR | A | 19 | 27.156 | 22.040 | 92.945 | 1.00 | 0.00 | xxxx | 151 |
| ATOM | 152 | O | THR | A | 19 | 27.605 | 22.608 | 93.946 | 1.00 | 0.00 | xxxx | 152 |
| ATOM | 153 | CB | THR | A | 19 | 24.937 | 22.719 | 91.970 | 1.00 | 0.00 | xxxx | 153 |
| ATOM | 154 | OG1 | THR | A | 19 | 24.344 | 23.589 | 90.996 | 1.00 | 0.00 | xxxx | 154 |
| ATOM | 155 | CG2 | THR | A | 19 | 24.485 | 23.130 | 93.358 | 1.00 | 0.00 | xxxx | 155 |
| ATOM | 156 | N | GLY | A | 20 | 27.297 | 20.729 | 92.755 | 1.00 | 0.00 | xxxx | 156 |
| ATOM | 157 | CA | GLY | A | 20 | 28.061 | 19.945 | 93.709 | 1.00 | 0.00 | xxxx | 157 |
| ATOM | 158 | C | GLY | A | 20 | 29.516 | 20.366 | 93.780 | 1.00 | 0.00 | xxxx | 158 |
| ATOM | 159 | O | GLY | A | 20 | 30.103 | 20.412 | 94.866 | 1.00 | 0.00 | xxxx | 159 |
| ATOM | 160 | N | VAL | A | 21 | 30.118 | 20.682 | 92.628 | 1.00 | 0.00 | xxxx | 160 |
| ATOM | 161 | CA | VAL | A | 21 | 31.509 | 21.135 | 92.614 | 1.00 | 0.00 | xxxx | 161 |
| ATOM | 162 | C | VAL | A | 21 | 31.650 | 22.459 | 93.348 | 1.00 | 0.00 | xxxx | 162 |
| ATOM | 163 | O | VAL | A | 21 | 32.525 | 22.622 | 94.203 | 1.00 | 0.00 | xxxx | 163 |
| ATOM | 164 | CB | VAL | A | 21 | 32.041 | 21.238 | 91.175 | 1.00 | 0.00 | xxxx | 164 |
| ATOM | 165 | CG1 | VAL | A | 21 | 33.437 | 21.844 | 91.180 | 1.00 | 0.00 | xxxx | 165 |
| ATOM | 166 | CG2 | VAL | A | 21 | 32.058 | 19.874 | 90.497 | 1.00 | 0.00 | xxxx | 166 |
| ATOM | 167 | N | ARG | A | 22 | 30.810 | 23.440 | 93.015 | 1.00 | 0.00 | xxxx | 167 |
| ATOM | 168 | CA | ARG | A | 22 | 31.006 | 24.739 | 93.649 | 1.00 | 0.00 | xxxx | 168 |
| ATOM | 169 | C | ARG | A | 22 | 30.718 | 24.699 | 95.149 | 1.00 | 0.00 | xxxx | 169 |
| ATOM | 170 | O | ARG | A | 22 | 31.406 | 25.379 | 95.918 | 1.00 | 0.00 | xxxx | 170 |
| ATOM | 171 | CB | ARG | A | 22 | 30.269 | 25.848 | 92.897 | 1.00 | 0.00 | xxxx | 171 |
| ATOM | 172 | CG | ARG | A | 22 | 28.782 | 25.708 | 92.836 | 1.00 | 0.00 | xxxx | 172 |
| ATOM | 173 | CD | ARG | A | 22 | 28.239 | 26.768 | 91.886 | 1.00 | 0.00 | xxxx | 173 |
| ATOM | 174 | NE | ARG | A | 22 | 26.839 | 26.539 | 91.568 | 1.00 | 0.00 | xxxx | 174 |
| ATOM | 175 | CZ | ARG | A | 22 | 25.837 | 26.992 | 92.305 | 1.00 | 0.00 | xxxx | 175 |
| ATOM | 176 | NH1 | ARG | A | 22 | 26.091 | 27.690 | 93.407 | 1.00 | 0.00 | xxxx | 176 |
| ATOM | 177 | NH2 | ARG | A | 22 | 24.585 | 26.746 | 91.952 | 1.00 | 0.00 | xxxx | 177 |
| ATOM | 178 | N | ASN | A | 23 | 29.746 | 23.897 | 95.592 | 1.00 | 0.00 | xxxx | 178 |
| ATOM | 179 | CA | ASN | A | 23 | 29.515 | 23.756 | 97.027 | 1.00 | 0.00 | xxxx | 179 |
| ATOM | 180 | C | ASN | A | 23 | 30.709 | 23.111 | 97.717 | 1.00 | 0.00 | xxxx | 180 |
| ATOM | 181 | O | ASN | A | 23 | 31.083 | 23.507 | 98.825 | 1.00 | 0.00 | xxxx | 181 |
| ATOM | 182 | CB | ASN | A | 23 | 28.260 | 22.929 | 97.291 | 1.00 | 0.00 | xxxx | 182 |
| ATOM | 183 | CG | ASN | A | 23 | 26.988 | 23.666 | 96.932 | 1.00 | 0.00 | xxxx | 183 |
| ATOM | 184 | OD1 | ASN | A | 23 | 26.978 | 24.886 | 96.797 | 1.00 | 0.00 | xxxx | 184 |
| ATOM | 185 | ND2 | ASN | A | 23 | 25.905 | 22.922 | 96.777 | 1.00 | 0.00 | xxxx | 185 |
| ATOM | 186 | N | ALA | A | 24 | 31.328 | 22.119 | 97.076 | 1.00 | 0.00 | xxxx | 186 |
| ATOM | 187 | CA | ALA | A | 24 | 32.500 | 21.497 | 97.682 | 1.00 | 0.00 | xxxx | 187 |
| ATOM | 188 | C | ALA | A | 24 | 33.708 | 22.429 | 97.666 | 1.00 | 0.00 | xxxx | 188 |
| ATOM | 189 | O | ALA | A | 24 | 34.502 | 22.416 | 98.615 | 1.00 | 0.00 | xxxx | 189 |
| ATOM | 190 | CB | ALA | A | 24 | 32.809 | 20.168 | 96.998 | 1.00 | 0.00 | xxxx | 190 |
| ATOM | 191 | N | MET | A | 25 | 33.850 | 23.259 | 96.627 | 1.00 | 0.00 | xxxx | 191 |
| ATOM | 192 | CA | MET | A | 25 | 34.940 | 24.231 | 96.613 | 1.00 | 0.00 | xxxx | 192 |
| ATOM | 193 | C | MET | A | 25 | 34.787 | 25.222 | 97.753 | 1.00 | 0.00 | xxxx | 193 |
| ATOM | 194 | O | MET | A | 25 | 35.764 | 25.546 | 98.437 | 1.00 | 0.00 | xxxx | 194 |
| ATOM | 195 | CB | MET | A | 25 | 34.981 | 24.968 | 95.270 | 1.00 | 0.00 | xxxx | 195 |
| ATOM | 196 | CG | MET | A | 25 | 35.449 | 24.116 | 94.095 | 1.00 | 0.00 | xxxx | 196 |
| ATOM | 197 | SD | MET | A | 25 | 35.613 | 25.003 | 92.534 | 1.00 | 0.00 | xxxx | 197 |
| ATOM | 198 | CE | MET | A | 25 | 36.694 | 26.323 | 93.078 | 1.00 | 0.00 | xxxx | 198 |
| ATOM | 199 | N | THR | A | 26 | 33.564 | 25.721 | 97.964 | 1.00 | 0.00 | xxxx | 199 |
| ATOM | 200 | CA | THR | A | 26 | 33.301 | 26.646 | 99.061 | 1.00 | 0.00 | xxxx | 200 |
| ATOM | 201 | C | THR | A | 26 | 33.669 | 26.015 | 100.393 | 1.00 | 0.00 | xxxx | 201 |
| ATOM | 202 | O | THR | A | 26 | 34.274 | 26.667 | 101.256 | 1.00 | 0.00 | xxxx | 202 |
| ATOM | 203 | CB | THR | A | 26 | 31.825 | 27.039 | 99.033 | 1.00 | 0.00 | xxxx | 203 |
| ATOM | 204 | OG1 | THR | A | 26 | 31.547 | 27.723 | 97.806 | 1.00 | 0.00 | xxxx | 204 |
| ATOM | 205 | CG2 | THR | A | 26 | 31.469 | 27.953 | 100.202 | 1.00 | 0.00 | xxxx | 205 |
| ATOM | 206 | N | ALA | A | 27 | 33.338 | 24.735 | 100.573 | 1.00 | 0.00 | xxxx | 206 |
| ATOM | 207 | CA | ALA | A | 27 | 33.658 | 24.071 | 101.828 | 1.00 | 0.00 | xxxx | 207 |
| ATOM | 208 | C | ALA | A | 27 | 35.165 | 23.922 | 102.008 | 1.00 | 0.00 | xxxx | 208 |
| ATOM | 209 | O | ALA | A | 27 | 35.680 | 24.097 | 103.116 | 1.00 | 0.00 | xxxx | 209 |
| ATOM | 210 | CB | ALA | A | 27 | 32.957 | 22.717 | 101.889 | 1.00 | 0.00 | xxxx | 210 |
| ATOM | 211 | N | GLU | A | 28 | 35.892 | 23.621 | 100.930 | 1.00 | 0.00 | xxxx | 211 |
| ATOM | 212 | CA | GLU | A | 28 | 37.337 | 23.457 | 101.064 | 1.00 | 0.00 | xxxx | 212 |
| ATOM | 213 | C | GLU | A | 28 | 38.032 | 24.785 | 101.340 | 1.00 | 0.00 | xxxx | 213 |
| ATOM | 214 | O | GLU | A | 28 | 39.046 | 24.814 | 102.045 | 1.00 | 0.00 | xxxx | 214 |
| ATOM | 215 | CB | GLU | A | 28 | 37.917 | 22.797 | 99.812 | 1.00 | 0.00 | xxxx | 215 |
| ATOM | 216 | CG | GLU | A | 28 | 39.357 | 22.325 | 99.980 | 1.00 | 0.00 | xxxx | 216 |
| ATOM | 217 | CD | GLU | A | 28 | 39.498 | 21.215 | 101.005 | 1.00 | 0.00 | xxxx | 217 |
| ATOM | 218 | OE1 | GLU | A | 28 | 38.534 | 20.443 | 101.189 | 1.00 | 0.00 | xxxx | 218 |
| ATOM | 219 | OE2 | GLU | A | 28 | 40.576 | 21.116 | 101.631 | 1.00 | 0.00 | xxxx | 219 |
| ATOM | 220 | N | ALA | A | 29 | 37.501 | 25.884 | 100.801 | 1.00 | 0.00 | xxxx | 220 |
| ATOM | 221 | CA | ALA | A | 29 | 38.119 | 27.195 | 100.943 | 1.00 | 0.00 | xxxx | 221 |
| ATOM | 222 | C | ALA | A | 29 | 37.903 | 27.814 | 102.314 | 1.00 | 0.00 | xxxx | 222 |
| ATOM | 223 | O | ALA | A | 29 | 38.651 | 28.725 | 102.677 | 1.00 | 0.00 | xxxx | 223 |
| ATOM | 224 | CB | ALA | A | 29 | 37.566 | 28.156 | 99.889 | 1.00 | 0.00 | xxxx | 224 |
| ATOM | 225 | N | GLN | A | 30 | 36.902 | 27.355 | 103.065 | 1.00 | 0.00 | xxxx | 225 |
| ATOM | 226 | CA | GLN | A | 30 | 36.545 | 27.956 | 104.346 | 1.00 | 0.00 | xxxx | 226 |
| ATOM | 227 | C | GLN | A | 30 | 37.751 | 28.033 | 105.270 | 1.00 | 0.00 | xxxx | 227 |
| ATOM | 228 | O | GLN | A | 30 | 38.410 | 27.024 | 105.533 | 1.00 | 0.00 | xxxx | 228 |
| ATOM | 229 | CB | GLN | A | 30 | 35.435 | 27.124 | 104.995 | 1.00 | 0.00 | xxxx | 229 |
| ATOM | 230 | CG | GLN | A | 30 | 34.875 | 27.717 | 106.281 | 1.00 | 0.00 | xxxx | 230 |
| ATOM | 231 | CD | GLN | A | 30 | 33.897 | 26.782 | 106.972 | 1.00 | 0.00 | xxxx | 231 |
| ATOM | 232 | OE1 | GLN | A | 30 | 34.295 | 25.762 | 107.534 | 1.00 | 0.00 | xxxx | 232 |
| ATOM | 233 | NE2 | GLN | A | 30 | 32.610 | 27.120 | 106.922 | 1.00 | 0.00 | xxxx | 233 |
| ATOM | 234 | N | GLY | A | 31 | 38.042 | 29.243 | 105.757 | 1.00 | 0.00 | xxxx | 234 |
| ATOM | 235 | CA | GLY | A | 31 | 39.175 | 29.474 | 106.631 | 1.00 | 0.00 | xxxx | 235 |
| ATOM | 236 | C | GLY | A | 31 | 40.523 | 29.560 | 105.946 | 1.00 | 0.00 | xxxx | 236 |
| ATOM | 237 | O | GLY | A | 31 | 41.533 | 29.751 | 106.633 | 1.00 | 0.00 | xxxx | 237 |
| ATOM | 238 | N | LYS | A | 32 | 40.578 | 29.446 | 104.618 | 1.00 | 0.00 | xxxx | 238 |
| ATOM | 239 | CA | LYS | A | 32 | 41.841 | 29.380 | 103.886 | 1.00 | 0.00 | xxxx | 239 |
| ATOM | 240 | C | LYS | A | 32 | 42.007 | 30.507 | 102.883 | 1.00 | 0.00 | xxxx | 240 |
| ATOM | 241 | O | LYS | A | 32 | 43.072 | 31.130 | 102.829 | 1.00 | 0.00 | xxxx | 241 |
| ATOM | 242 | CB | LYS | A | 32 | 41.971 | 28.034 | 103.156 | 1.00 | 0.00 | xxxx | 242 |
| ATOM | 243 | CG | LYS | A | 32 | 42.024 | 26.831 | 104.078 | 1.00 | 0.00 | xxxx | 243 |
| ATOM | 244 | CD | LYS | A | 32 | 42.100 | 25.541 | 103.276 | 1.00 | 0.00 | xxxx | 244 |
| ATOM | 245 | CE | LYS | A | 32 | 41.788 | 24.341 | 104.156 | 1.00 | 0.00 | xxxx | 245 |
| ATOM | 246 | NZ | LYS | A | 32 | 41.472 | 23.132 | 103.346 | 1.00 | 0.00 | xxxx | 246 |
| ATOM | 247 | N | ALA | A | 33 | 40.990 | 30.782 | 102.079 | 1.00 | 0.00 | xxxx | 247 |
| ATOM | 248 | CA | ALA | A | 33 | 41.101 | 31.780 | 101.029 | 1.00 | 0.00 | xxxx | 248 |
| ATOM | 249 | C | ALA | A | 33 | 39.702 | 32.228 | 100.659 | 1.00 | 0.00 | xxxx | 249 |
| ATOM | 250 | O | ALA | A | 33 | 38.728 | 31.492 | 100.837 | 1.00 | 0.00 | xxxx | 250 |
| ATOM | 251 | CB | ALA | A | 33 | 41.816 | 31.214 | 99.799 | 1.00 | 0.00 | xxxx | 251 |
| ATOM | 252 | N | LYS | A | 34 | 39.615 | 33.438 | 100.121 | 1.00 | 0.00 | xxxx | 252 |
| ATOM | 253 | CA | LYS | A | 34 | 38.337 | 34.001 | 99.709 | 1.00 | 0.00 | xxxx | 253 |
| ATOM | 254 | C | LYS | A | 34 | 38.080 | 33.631 | 98.255 | 1.00 | 0.00 | xxxx | 254 |
| ATOM | 255 | O | LYS | A | 34 | 38.876 | 33.970 | 97.373 | 1.00 | 0.00 | xxxx | 255 |
| ATOM | 256 | CB | LYS | A | 34 | 38.348 | 35.519 | 99.870 | 1.00 | 0.00 | xxxx | 256 |
| ATOM | 257 | CG | LYS | A | 34 | 37.036 | 36.178 | 99.475 | 1.00 | 0.00 | xxxx | 257 |
| ATOM | 258 | CD | LYS | A | 34 | 37.045 | 37.663 | 99.792 | 1.00 | 0.00 | xxxx | 258 |
| ATOM | 259 | CE | LYS | A | 34 | 35.786 | 38.332 | 99.252 | 1.00 | 0.00 | xxxx | 259 |
| ATOM | 260 | NZ | LYS | A | 34 | 35.790 | 39.805 | 99.460 | 1.00 | 0.00 | xxxx | 260 |
| ATOM | 261 | N | LEU | A | 35 | 36.987 | 32.912 | 98.007 | 1.00 | 0.00 | xxxx | 261 |
| ATOM | 262 | CA | LEU | A | 35 | 36.547 | 32.647 | 96.645 | 1.00 | 0.00 | xxxx | 262 |
| ATOM | 263 | C | LEU | A | 35 | 35.617 | 33.769 | 96.220 | 1.00 | 0.00 | xxxx | 263 |
| ATOM | 264 | O | LEU | A | 35 | 34.622 | 34.052 | 96.898 | 1.00 | 0.00 | xxxx | 264 |
| ATOM | 265 | CB | LEU | A | 35 | 35.817 | 31.311 | 96.533 | 1.00 | 0.00 | xxxx | 265 |
| ATOM | 266 | CG | LEU | A | 35 | 36.570 | 30.079 | 97.011 | 1.00 | 0.00 | xxxx | 266 |
| ATOM | 267 | CD1 | LEU | A | 35 | 35.771 | 28.820 | 96.682 | 1.00 | 0.00 | xxxx | 267 |
| ATOM | 268 | CD2 | LEU | A | 35 | 37.962 | 30.024 | 96.405 | 1.00 | 0.00 | xxxx | 268 |
| ATOM | 269 | N | ASN | A | 36 | 35.946 | 34.414 | 95.113 | 1.00 | 0.00 | xxxx | 269 |
| ATOM | 270 | CA | ASN | A | 36 | 35.060 | 35.395 | 94.494 | 1.00 | 0.00 | xxxx | 270 |
| ATOM | 271 | C | ASN | A | 36 | 34.382 | 34.638 | 93.360 | 1.00 | 0.00 | xxxx | 271 |
| ATOM | 272 | O | ASN | A | 36 | 34.899 | 34.555 | 92.243 | 1.00 | 0.00 | xxxx | 272 |
| ATOM | 273 | CB | ASN | A | 36 | 35.870 | 36.593 | 94.027 | 1.00 | 0.00 | xxxx | 273 |
| ATOM | 274 | CG | ASN | A | 36 | 36.557 | 37.289 | 95.182 | 1.00 | 0.00 | xxxx | 274 |
| ATOM | 275 | OD1 | ASN | A | 36 | 35.957 | 38.126 | 95.846 | 1.00 | 0.00 | xxxx | 275 |
| ATOM | 276 | ND2 | ASN | A | 36 | 37.793 | 36.897 | 95.469 | 1.00 | 0.00 | xxxx | 276 |
| ATOM | 277 | N | MET | A | 37 | 33.217 | 34.063 | 93.641 | 1.00 | 0.00 | xxxx | 277 |
| ATOM | 278 | CA | MET | A | 37 | 32.609 | 33.085 | 92.743 | 1.00 | 0.00 | xxxx | 278 |
| ATOM | 279 | C | MET | A | 37 | 31.433 | 33.697 | 91.990 | 1.00 | 0.00 | xxxx | 279 |
| ATOM | 280 | O | MET | A | 37 | 30.486 | 34.207 | 92.603 | 1.00 | 0.00 | xxxx | 280 |
| ATOM | 281 | CB | MET | A | 37 | 32.180 | 31.831 | 93.499 | 1.00 | 0.00 | xxxx | 281 |
| ATOM | 282 | CG | MET | A | 37 | 31.663 | 30.735 | 92.582 | 1.00 | 0.00 | xxxx | 282 |
| ATOM | 283 | SD | MET | A | 37 | 31.040 | 29.361 | 93.555 | 1.00 | 0.00 | xxxx | 283 |
| ATOM | 284 | CE | MET | A | 37 | 32.585 | 28.543 | 93.944 | 1.00 | 0.00 | xxxx | 284 |
| ATOM | 285 | N | VAL | A | 38 | 31.493 | 33.622 | 90.662 | 1.00 | 0.00 | xxxx | 285 |
| ATOM | 286 | CA | VAL | A | 38 | 30.527 | 34.240 | 89.764 | 1.00 | 0.00 | xxxx | 286 |
| ATOM | 287 | C | VAL | A | 38 | 29.901 | 33.185 | 88.859 | 1.00 | 0.00 | xxxx | 287 |
| ATOM | 288 | O | VAL | A | 38 | 30.500 | 32.143 | 88.569 | 1.00 | 0.00 | xxxx | 288 |
| ATOM | 289 | CB | VAL | A | 38 | 31.133 | 35.399 | 88.929 | 1.00 | 0.00 | xxxx | 289 |
| ATOM | 290 | CG1 | VAL | A | 38 | 31.703 | 36.485 | 89.833 | 1.00 | 0.00 | xxxx | 290 |
| ATOM | 291 | CG2 | VAL | A | 38 | 32.187 | 34.886 | 87.965 | 1.00 | 0.00 | xxxx | 291 |
| ATOM | 292 | N | ASP | A | 39 | 28.679 | 33.478 | 88.428 | 1.00 | 0.00 | xxxx | 292 |
| ATOM | 293 | CA | ASP | A | 39 | 27.868 | 32.649 | 87.543 | 1.00 | 0.00 | xxxx | 293 |
| ATOM | 294 | C | ASP | A | 39 | 27.994 | 33.221 | 86.135 | 1.00 | 0.00 | xxxx | 294 |
| ATOM | 295 | O | ASP | A | 39 | 27.584 | 34.359 | 85.879 | 1.00 | 0.00 | xxxx | 295 |
| ATOM | 296 | CB | ASP | A | 39 | 26.413 | 32.740 | 88.030 | 1.00 | 0.00 | xxxx | 296 |
| ATOM | 297 | CG | ASP | A | 39 | 25.435 | 31.916 | 87.197 | 1.00 | 0.00 | xxxx | 297 |
| ATOM | 298 | OD1 | ASP | A | 39 | 25.864 | 31.210 | 86.273 | 1.00 | 0.00 | xxxx | 298 |
| ATOM | 299 | OD2 | ASP | A | 39 | 24.217 | 31.984 | 87.482 | 1.00 | 0.00 | xxxx | 299 |
| ATOM | 300 | N | SER | A | 40 | 28.582 | 32.448 | 85.224 | 1.00 | 0.00 | xxxx | 300 |
| ATOM | 301 | CA | SER | A | 40 | 28.718 | 32.933 | 83.852 | 1.00 | 0.00 | xxxx | 301 |
| ATOM | 302 | C | SER | A | 40 | 27.437 | 32.805 | 83.040 | 1.00 | 0.00 | xxxx | 302 |
| ATOM | 303 | O | SER | A | 40 | 27.412 | 33.251 | 81.888 | 1.00 | 0.00 | xxxx | 303 |
| ATOM | 304 | CB | SER | A | 40 | 29.876 | 32.232 | 83.136 | 1.00 | 0.00 | xxxx | 304 |
| ATOM | 305 | OG | SER | A | 40 | 31.102 | 32.449 | 83.831 | 1.00 | 0.00 | xxxx | 305 |
| ATOM | 306 | N | GLN | A | 41 | 26.383 | 32.212 | 83.608 | 1.00 | 0.00 | xxxx | 306 |
| ATOM | 307 | CA | GLN | A | 41 | 25.049 | 32.190 | 83.005 | 1.00 | 0.00 | xxxx | 307 |
| ATOM | 308 | C | GLN | A | 41 | 25.053 | 31.588 | 81.602 | 1.00 | 0.00 | xxxx | 308 |
| ATOM | 309 | O | GLN | A | 41 | 24.299 | 32.010 | 80.720 | 1.00 | 0.00 | xxxx | 309 |
| ATOM | 310 | CB | GLN | A | 41 | 24.394 | 33.572 | 83.017 | 1.00 | 0.00 | xxxx | 310 |
| ATOM | 311 | CG | GLN | A | 41 | 24.215 | 34.163 | 84.415 | 1.00 | 0.00 | xxxx | 311 |
| ATOM | 312 | CD | GLN | A | 41 | 23.401 | 35.430 | 84.386 | 1.00 | 0.00 | xxxx | 312 |
| ATOM | 313 | OE1 | GLN | A | 41 | 23.931 | 36.521 | 84.568 | 1.00 | 0.00 | xxxx | 313 |
| ATOM | 314 | NE2 | GLN | A | 41 | 22.103 | 35.294 | 84.144 | 1.00 | 0.00 | xxxx | 314 |
| ATOM | 315 | N | ASN | A | 42 | 25.921 | 30.598 | 81.388 | 1.00 | 0.00 | xxxx | 315 |
| ATOM | 316 | CA | ASN | A | 42 | 25.990 | 29.866 | 80.122 | 1.00 | 0.00 | xxxx | 316 |
| ATOM | 317 | C | ASN | A | 42 | 26.345 | 30.772 | 78.942 | 1.00 | 0.00 | xxxx | 317 |
| ATOM | 318 | O | ASN | A | 42 | 25.944 | 30.504 | 77.809 | 1.00 | 0.00 | xxxx | 318 |
| ATOM | 319 | CB | ASN | A | 42 | 24.694 | 29.085 | 79.850 | 1.00 | 0.00 | xxxx | 319 |
| ATOM | 320 | CG | ASN | A | 42 | 24.907 | 27.888 | 78.954 | 1.00 | 0.00 | xxxx | 320 |
| ATOM | 321 | OD1 | ASN | A | 42 | 23.973 | 27.408 | 78.298 | 1.00 | 0.00 | xxxx | 321 |
| ATOM | 322 | ND2 | ASN | A | 42 | 26.124 | 27.401 | 78.908 | 1.00 | 0.00 | xxxx | 322 |
| ATOM | 323 | N | SER | A | 43 | 27.103 | 31.840 | 79.204 | 1.00 | 0.00 | xxxx | 323 |
| ATOM | 324 | CA | SER | A | 43 | 27.490 | 32.810 | 78.185 | 1.00 | 0.00 | xxxx | 324 |
| ATOM | 325 | C | SER | A | 43 | 28.996 | 33.028 | 78.247 | 1.00 | 0.00 | xxxx | 325 |
| ATOM | 326 | O | SER | A | 43 | 29.512 | 33.541 | 79.245 | 1.00 | 0.00 | xxxx | 326 |
| ATOM | 327 | CB | SER | A | 43 | 26.766 | 34.134 | 78.438 | 1.00 | 0.00 | xxxx | 327 |
| ATOM | 328 | OG | SER | A | 43 | 27.295 | 35.177 | 77.627 | 1.00 | 0.00 | xxxx | 328 |
| ATOM | 329 | N | GLN | A | 44 | 29.707 | 32.653 | 77.183 | 1.00 | 0.00 | xxxx | 329 |
| ATOM | 330 | CA | GLN | A | 44 | 31.145 | 32.903 | 77.183 | 1.00 | 0.00 | xxxx | 330 |
| ATOM | 331 | C | GLN | A | 44 | 31.464 | 34.397 | 77.155 | 1.00 | 0.00 | xxxx | 331 |
| ATOM | 332 | O | GLN | A | 44 | 32.416 | 34.810 | 77.829 | 1.00 | 0.00 | xxxx | 332 |
| ATOM | 333 | CB | GLN | A | 44 | 31.877 | 32.158 | 76.060 | 1.00 | 0.00 | xxxx | 333 |
| ATOM | 334 | CG | GLN | A | 44 | 33.404 | 32.203 | 76.217 | 1.00 | 0.00 | xxxx | 334 |
| ATOM | 335 | CD | GLN | A | 44 | 33.891 | 31.519 | 77.490 | 1.00 | 0.00 | xxxx | 335 |
| ATOM | 336 | OE1 | GLN | A | 44 | 33.482 | 30.407 | 77.804 | 1.00 | 0.00 | xxxx | 336 |
| ATOM | 337 | NE2 | GLN | A | 44 | 34.769 | 32.193 | 78.233 | 1.00 | 0.00 | xxxx | 337 |
| ATOM | 338 | N | PRO | A | 45 | 30.726 | 35.240 | 76.409 | 1.00 | 0.00 | xxxx | 338 |
| ATOM | 339 | CA | PRO | A | 45 | 30.953 | 36.693 | 76.549 | 1.00 | 0.00 | xxxx | 339 |
| ATOM | 340 | C | PRO | A | 45 | 30.815 | 37.191 | 77.980 | 1.00 | 0.00 | xxxx | 340 |
| ATOM | 341 | O | PRO | A | 45 | 31.629 | 38.014 | 78.421 | 1.00 | 0.00 | xxxx | 341 |
| ATOM | 342 | CB | PRO | A | 45 | 29.924 | 37.307 | 75.589 | 1.00 | 0.00 | xxxx | 342 |
| ATOM | 343 | CG | PRO | A | 45 | 29.751 | 36.260 | 74.530 | 1.00 | 0.00 | xxxx | 343 |
| ATOM | 344 | CD | PRO | A | 45 | 29.797 | 34.957 | 75.292 | 1.00 | 0.00 | xxxx | 344 |
| ATOM | 345 | N | THR | A | 46 | 29.825 | 36.701 | 78.730 | 1.00 | 0.00 | xxxx | 345 |
| ATOM | 346 | CA | THR | A | 46 | 29.725 | 37.065 | 80.142 | 1.00 | 0.00 | xxxx | 346 |
| ATOM | 347 | C | THR | A | 46 | 30.965 | 36.622 | 80.903 | 1.00 | 0.00 | xxxx | 347 |
| ATOM | 348 | O | THR | A | 46 | 31.536 | 37.383 | 81.693 | 1.00 | 0.00 | xxxx | 348 |
| ATOM | 349 | CB | THR | A | 46 | 28.468 | 36.445 | 80.753 | 1.00 | 0.00 | xxxx | 349 |
| ATOM | 350 | OG1 | THR | A | 46 | 27.309 | 36.962 | 80.093 | 1.00 | 0.00 | xxxx | 350 |
| ATOM | 351 | CG2 | THR | A | 46 | 28.374 | 36.765 | 82.240 | 1.00 | 0.00 | xxxx | 351 |
| ATOM | 352 | N | GLN | A | 47 | 31.397 | 35.377 | 80.687 | 1.00 | 0.00 | xxxx | 352 |
| ATOM | 353 | CA | GLN | A | 47 | 32.586 | 34.892 | 81.372 | 1.00 | 0.00 | xxxx | 353 |
| ATOM | 354 | C | GLN | A | 47 | 33.819 | 35.708 | 81.003 | 1.00 | 0.00 | xxxx | 354 |
| ATOM | 355 | O | GLN | A | 47 | 34.664 | 35.984 | 81.863 | 1.00 | 0.00 | xxxx | 355 |
| ATOM | 356 | CB | GLN | A | 47 | 32.806 | 33.415 | 81.067 | 1.00 | 0.00 | xxxx | 356 |
| ATOM | 357 | CG | GLN | A | 47 | 33.924 | 32.840 | 81.894 | 1.00 | 0.00 | xxxx | 357 |
| ATOM | 358 | CD | GLN | A | 47 | 34.000 | 31.341 | 81.767 | 1.00 | 0.00 | xxxx | 358 |
| ATOM | 359 | OE1 | GLN | A | 47 | 34.729 | 30.823 | 80.926 | 1.00 | 0.00 | xxxx | 359 |
| ATOM | 360 | NE2 | GLN | A | 47 | 33.239 | 30.638 | 82.593 | 1.00 | 0.00 | xxxx | 360 |
| ATOM | 361 | N | ASN | A | 48 | 33.944 | 36.101 | 79.733 | 1.00 | 0.00 | xxxx | 361 |
| ATOM | 362 | CA | ASN | A | 48 | 35.106 | 36.886 | 79.329 | 1.00 | 0.00 | xxxx | 362 |
| ATOM | 363 | C | ASN | A | 48 | 35.147 | 38.210 | 80.081 | 1.00 | 0.00 | xxxx | 363 |
| ATOM | 364 | O | ASN | A | 48 | 36.215 | 38.640 | 80.526 | 1.00 | 0.00 | xxxx | 364 |
| ATOM | 365 | CB | ASN | A | 48 | 35.093 | 37.119 | 77.818 | 1.00 | 0.00 | xxxx | 365 |
| ATOM | 366 | CG | ASN | A | 48 | 35.338 | 35.844 | 77.022 | 1.00 | 0.00 | xxxx | 366 |
| ATOM | 367 | OD1 | ASN | A | 48 | 35.813 | 34.837 | 77.560 | 1.00 | 0.00 | xxxx | 367 |
| ATOM | 368 | ND2 | ASN | A | 48 | 35.005 | 35.879 | 75.742 | 1.00 | 0.00 | xxxx | 368 |
| ATOM | 369 | N | ASP | A | 49 | 33.984 | 38.847 | 80.264 | 1.00 | 0.00 | xxxx | 369 |
| ATOM | 370 | CA | ASP | A | 49 | 33.916 | 40.074 | 81.054 | 1.00 | 0.00 | xxxx | 370 |
| ATOM | 371 | C | ASP | A | 49 | 34.280 | 39.820 | 82.512 | 1.00 | 0.00 | xxxx | 371 |
| ATOM | 372 | O | ASP | A | 49 | 34.934 | 40.660 | 83.147 | 1.00 | 0.00 | xxxx | 372 |
| ATOM | 373 | CB | ASP | A | 49 | 32.508 | 40.675 | 80.986 | 1.00 | 0.00 | xxxx | 373 |
| ATOM | 374 | CG | ASP | A | 49 | 32.154 | 41.215 | 79.609 | 1.00 | 0.00 | xxxx | 374 |
| ATOM | 375 | OD1 | ASP | A | 49 | 33.076 | 41.514 | 78.817 | 1.00 | 0.00 | xxxx | 375 |
| ATOM | 376 | OD2 | ASP | A | 49 | 30.936 | 41.359 | 79.327 | 1.00 | 0.00 | xxxx | 376 |
| ATOM | 377 | N | GLN | A | 50 | 33.840 | 38.683 | 83.065 | 1.00 | 0.00 | xxxx | 377 |
| ATOM | 378 | CA | GLN | A | 50 | 34.152 | 38.357 | 84.456 | 1.00 | 0.00 | xxxx | 378 |
| ATOM | 379 | C | GLN | A | 50 | 35.644 | 38.123 | 84.643 | 1.00 | 0.00 | xxxx | 379 |
| ATOM | 380 | O | GLN | A | 50 | 36.229 | 38.561 | 85.640 | 1.00 | 0.00 | xxxx | 380 |
| ATOM | 381 | CB | GLN | A | 50 | 33.346 | 37.126 | 84.890 | 1.00 | 0.00 | xxxx | 381 |
| ATOM | 382 | CG | GLN | A | 50 | 31.866 | 37.420 | 85.041 | 1.00 | 0.00 | xxxx | 382 |
| ATOM | 383 | CD | GLN | A | 50 | 30.984 | 36.172 | 85.027 | 1.00 | 0.00 | xxxx | 383 |
| ATOM | 384 | OE1 | GLN | A | 50 | 31.348 | 35.136 | 84.463 | 1.00 | 0.00 | xxxx | 384 |
| ATOM | 385 | NE2 | GLN | A | 50 | 29.819 | 36.278 | 85.644 | 1.00 | 0.00 | xxxx | 385 |
| ATOM | 386 | N | VAL | A | 51 | 36.277 | 37.439 | 83.691 | 1.00 | 0.00 | xxxx | 386 |
| ATOM | 387 | CA | VAL | A | 51 | 37.723 | 37.252 | 83.744 | 1.00 | 0.00 | xxxx | 387 |
| ATOM | 388 | C | VAL | A | 51 | 38.437 | 38.594 | 83.676 | 1.00 | 0.00 | xxxx | 388 |
| ATOM | 389 | O | VAL | A | 51 | 39.393 | 38.843 | 84.424 | 1.00 | 0.00 | xxxx | 389 |
| ATOM | 390 | CB | VAL | A | 51 | 38.168 | 36.313 | 82.613 | 1.00 | 0.00 | xxxx | 390 |
| ATOM | 391 | CG1 | VAL | A | 51 | 39.692 | 36.311 | 82.501 | 1.00 | 0.00 | xxxx | 391 |
| ATOM | 392 | CG2 | VAL | A | 51 | 37.640 | 34.914 | 82.858 | 1.00 | 0.00 | xxxx | 392 |
| ATOM | 393 | N | ASP | A | 52 | 37.987 | 39.480 | 82.783 | 1.00 | 0.00 | xxxx | 393 |
| ATOM | 394 | CA | ASP | A | 52 | 38.587 | 40.807 | 82.716 | 1.00 | 0.00 | xxxx | 394 |
| ATOM | 395 | C | ASP | A | 52 | 38.515 | 41.507 | 84.069 | 1.00 | 0.00 | xxxx | 395 |
| ATOM | 396 | O | ASP | A | 52 | 39.484 | 42.144 | 84.500 | 1.00 | 0.00 | xxxx | 396 |
| ATOM | 397 | CB | ASP | A | 52 | 37.899 | 41.657 | 81.645 | 1.00 | 0.00 | xxxx | 397 |
| ATOM | 398 | CG | ASP | A | 52 | 38.259 | 41.239 | 80.228 | 1.00 | 0.00 | xxxx | 398 |
| ATOM | 399 | OD1 | ASP | A | 52 | 39.185 | 40.416 | 80.046 | 1.00 | 0.00 | xxxx | 399 |
| ATOM | 400 | OD2 | ASP | A | 52 | 37.614 | 41.754 | 79.288 | 1.00 | 0.00 | xxxx | 400 |
| ATOM | 401 | N | LEU | A | 53 | 37.374 | 41.403 | 84.754 | 1.00 | 0.00 | xxxx | 401 |
| ATOM | 402 | CA | LEU | A | 53 | 37.227 | 42.087 | 86.038 | 1.00 | 0.00 | xxxx | 402 |
| ATOM | 403 | C | LEU | A | 53 | 38.088 | 41.440 | 87.121 | 1.00 | 0.00 | xxxx | 403 |
| ATOM | 404 | O | LEU | A | 53 | 38.674 | 42.141 | 87.954 | 1.00 | 0.00 | xxxx | 404 |
| ATOM | 405 | CB | LEU | A | 53 | 35.758 | 42.117 | 86.454 | 1.00 | 0.00 | xxxx | 405 |
| ATOM | 406 | CG | LEU | A | 53 | 35.448 | 42.973 | 87.687 | 1.00 | 0.00 | xxxx | 406 |
| ATOM | 407 | CD1 | LEU | A | 53 | 35.863 | 44.424 | 87.464 | 1.00 | 0.00 | xxxx | 407 |
| ATOM | 408 | CD2 | LEU | A | 53 | 33.967 | 42.886 | 88.029 | 1.00 | 0.00 | xxxx | 408 |
| ATOM | 409 | N | PHE | A | 54 | 38.176 | 40.109 | 87.135 | 1.00 | 0.00 | xxxx | 409 |
| ATOM | 410 | CA | PHE | A | 54 | 39.078 | 39.445 | 88.071 | 1.00 | 0.00 | xxxx | 410 |
| ATOM | 411 | C | PHE | A | 54 | 40.501 | 39.957 | 87.905 | 1.00 | 0.00 | xxxx | 411 |
| ATOM | 412 | O | PHE | A | 54 | 41.227 | 40.139 | 88.889 | 1.00 | 0.00 | xxxx | 412 |
| ATOM | 413 | CB | PHE | A | 54 | 39.098 | 37.940 | 87.795 | 1.00 | 0.00 | xxxx | 413 |
| ATOM | 414 | CG | PHE | A | 54 | 37.895 | 37.183 | 88.296 | 1.00 | 0.00 | xxxx | 414 |
| ATOM | 415 | CD1 | PHE | A | 54 | 37.168 | 37.619 | 89.387 | 1.00 | 0.00 | xxxx | 415 |
| ATOM | 416 | CD2 | PHE | A | 54 | 37.503 | 36.017 | 87.661 | 1.00 | 0.00 | xxxx | 416 |
| ATOM | 417 | CE1 | PHE | A | 54 | 36.072 | 36.896 | 89.840 | 1.00 | 0.00 | xxxx | 417 |
| ATOM | 418 | CE2 | PHE | A | 54 | 36.407 | 35.290 | 88.111 | 1.00 | 0.00 | xxxx | 418 |
| ATOM | 419 | CZ | PHE | A | 54 | 35.692 | 35.739 | 89.193 | 1.00 | 0.00 | xxxx | 419 |
| ATOM | 420 | N | ILE | A | 55 | 40.929 | 40.159 | 86.657 | 1.00 | 0.00 | xxxx | 420 |
| ATOM | 421 | CA | ILE | A | 55 | 42.280 | 40.634 | 86.395 | 1.00 | 0.00 | xxxx | 421 |
| ATOM | 422 | C | ILE | A | 55 | 42.449 | 42.073 | 86.874 | 1.00 | 0.00 | xxxx | 422 |
| ATOM | 423 | O | ILE | A | 55 | 43.450 | 42.413 | 87.519 | 1.00 | 0.00 | xxxx | 423 |
| ATOM | 424 | CB | ILE | A | 55 | 42.612 | 40.457 | 84.901 | 1.00 | 0.00 | xxxx | 424 |
| ATOM | 425 | CG1 | ILE | A | 55 | 42.783 | 38.969 | 84.590 | 1.00 | 0.00 | xxxx | 425 |
| ATOM | 426 | CG2 | ILE | A | 55 | 43.853 | 41.263 | 84.512 | 1.00 | 0.00 | xxxx | 426 |
| ATOM | 427 | CD1 | ILE | A | 55 | 42.900 | 38.654 | 83.114 | 1.00 | 0.00 | xxxx | 427 |
| ATOM | 428 | N | THR | A | 56 | 41.470 | 42.941 | 86.588 | 1.00 | 0.00 | xxxx | 428 |
| ATOM | 429 | CA | THR | A | 56 | 41.569 | 44.323 | 87.048 | 1.00 | 0.00 | xxxx | 429 |
| ATOM | 430 | C | THR | A | 56 | 41.538 | 44.409 | 88.569 | 1.00 | 0.00 | xxxx | 430 |
| ATOM | 431 | O | THR | A | 56 | 42.149 | 45.310 | 89.155 | 1.00 | 0.00 | xxxx | 431 |
| ATOM | 432 | CB | THR | A | 56 | 40.458 | 45.172 | 86.432 | 1.00 | 0.00 | xxxx | 432 |
| ATOM | 433 | OG1 | THR | A | 56 | 39.187 | 44.752 | 86.937 | 1.00 | 0.00 | xxxx | 433 |
| ATOM | 434 | CG2 | THR | A | 56 | 40.467 | 45.040 | 84.930 | 1.00 | 0.00 | xxxx | 434 |
| ATOM | 435 | N | LYS | A | 57 | 40.841 | 43.486 | 89.223 | 1.00 | 0.00 | xxxx | 435 |
| ATOM | 436 | CA | LYS | A | 57 | 40.787 | 43.451 | 90.680 | 1.00 | 0.00 | xxxx | 436 |
| ATOM | 437 | C | LYS | A | 57 | 41.975 | 42.728 | 91.294 | 1.00 | 0.00 | xxxx | 437 |
| ATOM | 438 | O | LYS | A | 57 | 42.015 | 42.567 | 92.519 | 1.00 | 0.00 | xxxx | 438 |
| ATOM | 439 | CB | LYS | A | 57 | 39.490 | 42.779 | 91.139 | 1.00 | 0.00 | xxxx | 439 |
| ATOM | 440 | CG | LYS | A | 57 | 38.236 | 43.606 | 90.893 | 1.00 | 0.00 | xxxx | 440 |
| ATOM | 441 | CD | LYS | A | 57 | 36.997 | 42.845 | 91.342 | 1.00 | 0.00 | xxxx | 441 |
| ATOM | 442 | CE | LYS | A | 57 | 35.754 | 43.721 | 91.300 | 1.00 | 0.00 | xxxx | 442 |
| ATOM | 443 | NZ | LYS | A | 57 | 34.543 | 42.975 | 91.742 | 1.00 | 0.00 | xxxx | 443 |
| ATOM | 444 | N | LYS | A | 58 | 42.920 | 42.268 | 90.474 | 1.00 | 0.00 | xxxx | 444 |
| ATOM | 445 | CA | LYS | A | 58 | 44.163 | 41.654 | 90.948 | 1.00 | 0.00 | xxxx | 445 |
| ATOM | 446 | C | LYS | A | 58 | 43.915 | 40.432 | 91.838 | 1.00 | 0.00 | xxxx | 446 |
| ATOM | 447 | O | LYS | A | 58 | 44.473 | 40.308 | 92.930 | 1.00 | 0.00 | xxxx | 447 |
| ATOM | 448 | CB | LYS | A | 58 | 45.084 | 42.677 | 91.621 | 1.00 | 0.00 | xxxx | 448 |
| ATOM | 449 | CG | LYS | A | 58 | 45.491 | 43.832 | 90.708 | 1.00 | 0.00 | xxxx | 449 |
| ATOM | 450 | CD | LYS | A | 58 | 46.466 | 44.767 | 91.415 | 1.00 | 0.00 | xxxx | 450 |
| ATOM | 451 | CE | LYS | A | 58 | 46.876 | 45.934 | 90.528 | 1.00 | 0.00 | xxxx | 451 |
| ATOM | 452 | NZ | LYS | A | 58 | 47.689 | 45.497 | 89.360 | 1.00 | 0.00 | xxxx | 452 |
| ATOM | 453 | N | MET | A | 59 | 43.076 | 39.515 | 91.355 | 1.00 | 0.00 | xxxx | 453 |
| ATOM | 454 | CA | MET | A | 59 | 42.977 | 38.209 | 91.994 | 1.00 | 0.00 | xxxx | 454 |
| ATOM | 455 | C | MET | A | 59 | 44.359 | 37.567 | 92.043 | 1.00 | 0.00 | xxxx | 455 |
| ATOM | 456 | O | MET | A | 59 | 45.208 | 37.809 | 91.177 | 1.00 | 0.00 | xxxx | 456 |
| ATOM | 457 | CB | MET | A | 59 | 42.041 | 37.296 | 91.195 | 1.00 | 0.00 | xxxx | 457 |
| ATOM | 458 | CG | MET | A | 59 | 40.570 | 37.704 | 91.162 | 1.00 | 0.00 | xxxx | 458 |
| ATOM | 459 | SD | MET | A | 59 | 39.731 | 37.646 | 92.761 | 1.00 | 0.00 | xxxx | 459 |
| ATOM | 460 | CE | MET | A | 59 | 39.788 | 39.371 | 93.232 | 1.00 | 0.00 | xxxx | 460 |
| ATOM | 461 | N | ASN | A | 60 | 44.589 | 36.749 | 93.080 | 1.00 | 0.00 | xxxx | 461 |
| ATOM | 462 | CA | ASN | A | 60 | 45.864 | 36.048 | 93.213 | 1.00 | 0.00 | xxxx | 462 |
| ATOM | 463 | C | ASN | A | 60 | 45.940 | 34.809 | 92.329 | 1.00 | 0.00 | xxxx | 463 |
| ATOM | 464 | O | ASN | A | 60 | 47.045 | 34.367 | 91.978 | 1.00 | 0.00 | xxxx | 464 |
| ATOM | 465 | CB | ASN | A | 60 | 46.117 | 35.663 | 94.677 | 1.00 | 0.00 | xxxx | 465 |
| ATOM | 466 | CG | ASN | A | 60 | 46.186 | 36.866 | 95.592 | 1.00 | 0.00 | xxxx | 466 |
| ATOM | 467 | OD1 | ASN | A | 60 | 45.232 | 37.173 | 96.299 | 1.00 | 0.00 | xxxx | 467 |
| ATOM | 468 | ND2 | ASN | A | 60 | 47.323 | 37.552 | 95.584 | 1.00 | 0.00 | xxxx | 468 |
| ATOM | 469 | N | ALA | A | 61 | 44.793 | 34.248 | 91.968 | 1.00 | 0.00 | xxxx | 469 |
| ATOM | 470 | CA | ALA | A | 61 | 44.730 | 33.123 | 91.049 | 1.00 | 0.00 | xxxx | 470 |
| ATOM | 471 | C | ALA | A | 61 | 43.314 | 33.070 | 90.501 | 1.00 | 0.00 | xxxx | 471 |
| ATOM | 472 | O | ALA | A | 61 | 42.387 | 33.651 | 91.072 | 1.00 | 0.00 | xxxx | 472 |
| ATOM | 473 | CB | ALA | A | 61 | 45.102 | 31.798 | 91.738 | 1.00 | 0.00 | xxxx | 473 |
| ATOM | 474 | N | LEU | A | 62 | 43.161 | 32.376 | 89.375 | 1.00 | 0.00 | xxxx | 474 |
| ATOM | 475 | CA | LEU | A | 62 | 41.862 | 32.166 | 88.747 | 1.00 | 0.00 | xxxx | 475 |
| ATOM | 476 | C | LEU | A | 62 | 41.563 | 30.678 | 88.674 | 1.00 | 0.00 | xxxx | 476 |
| ATOM | 477 | O | LEU | A | 62 | 42.460 | 29.874 | 88.422 | 1.00 | 0.00 | xxxx | 477 |
| ATOM | 478 | CB | LEU | A | 62 | 41.854 | 32.700 | 87.318 | 1.00 | 0.00 | xxxx | 478 |
| ATOM | 479 | CG | LEU | A | 62 | 42.259 | 34.157 | 87.163 | 1.00 | 0.00 | xxxx | 479 |
| ATOM | 480 | CD1 | LEU | A | 62 | 42.234 | 34.565 | 85.685 | 1.00 | 0.00 | xxxx | 480 |
| ATOM | 481 | CD2 | LEU | A | 62 | 41.333 | 35.015 | 87.996 | 1.00 | 0.00 | xxxx | 481 |
| ATOM | 482 | N | ALA | A | 63 | 40.294 | 30.331 | 88.884 | 1.00 | 0.00 | xxxx | 482 |
| ATOM | 483 | CA | ALA | A | 63 | 39.776 | 28.979 | 88.669 | 1.00 | 0.00 | xxxx | 483 |
| ATOM | 484 | C | ALA | A | 63 | 38.579 | 29.137 | 87.743 | 1.00 | 0.00 | xxxx | 484 |
| ATOM | 485 | O | ALA | A | 63 | 37.558 | 29.721 | 88.136 | 1.00 | 0.00 | xxxx | 485 |
| ATOM | 486 | CB | ALA | A | 63 | 39.374 | 28.307 | 89.990 | 1.00 | 0.00 | xxxx | 486 |
| ATOM | 487 | N | ILE | A | 64 | 38.705 | 28.644 | 86.509 | 1.00 | 0.00 | xxxx | 487 |
| ATOM | 488 | CA | ILE | A | 64 | 37.737 | 28.928 | 85.451 | 1.00 | 0.00 | xxxx | 488 |
| ATOM | 489 | C | ILE | A | 64 | 37.144 | 27.627 | 84.929 | 1.00 | 0.00 | xxxx | 489 |
| ATOM | 490 | O | ILE | A | 64 | 37.870 | 26.740 | 84.477 | 1.00 | 0.00 | xxxx | 490 |
| ATOM | 491 | CB | ILE | A | 64 | 38.361 | 29.719 | 84.285 | 1.00 | 0.00 | xxxx | 491 |
| ATOM | 492 | CG1 | ILE | A | 64 | 39.018 | 31.018 | 84.786 | 1.00 | 0.00 | xxxx | 492 |
| ATOM | 493 | CD1 | ILE | A | 64 | 38.075 | 31.990 | 85.470 | 1.00 | 0.00 | xxxx | 493 |
| ATOM | 494 | CG2 | ILE | A | 64 | 37.297 | 29.986 | 83.205 | 1.00 | 0.00 | xxxx | 494 |
| ATOM | 495 | N | ASN | A | 65 | 35.821 | 27.545 | 84.951 | 1.00 | 0.00 | xxxx | 495 |
| ATOM | 496 | CA | ASN | A | 65 | 35.047 | 26.466 | 84.354 | 1.00 | 0.00 | xxxx | 496 |
| ATOM | 497 | C | ASN | A | 65 | 34.469 | 27.053 | 83.070 | 1.00 | 0.00 | xxxx | 497 |
| ATOM | 498 | O | ASN | A | 65 | 33.449 | 27.756 | 83.123 | 1.00 | 0.00 | xxxx | 498 |
| ATOM | 499 | CB | ASN | A | 65 | 33.940 | 26.087 | 85.351 | 1.00 | 0.00 | xxxx | 499 |
| ATOM | 500 | CG | ASN | A | 65 | 32.918 | 25.098 | 84.818 | 1.00 | 0.00 | xxxx | 500 |
| ATOM | 501 | OD1 | ASN | A | 65 | 31.715 | 25.263 | 85.062 | 1.00 | 0.00 | xxxx | 501 |
| ATOM | 502 | ND2 | ASN | A | 65 | 33.364 | 24.068 | 84.105 | 1.00 | 0.00 | xxxx | 502 |
| ATOM | 503 | N | PRO | A | 66 | 35.101 | 26.838 | 81.906 | 1.00 | 0.00 | xxxx | 503 |
| ATOM | 504 | CA | PRO | A | 66 | 34.677 | 27.548 | 80.684 | 1.00 | 0.00 | xxxx | 504 |
| ATOM | 505 | C | PRO | A | 66 | 33.239 | 27.261 | 80.289 | 1.00 | 0.00 | xxxx | 505 |
| ATOM | 506 | O | PRO | A | 66 | 32.722 | 26.159 | 80.478 | 1.00 | 0.00 | xxxx | 506 |
| ATOM | 507 | CB | PRO | A | 66 | 35.636 | 27.022 | 79.610 | 1.00 | 0.00 | xxxx | 507 |
| ATOM | 508 | CG | PRO | A | 66 | 36.833 | 26.497 | 80.362 | 1.00 | 0.00 | xxxx | 508 |
| ATOM | 509 | CD | PRO | A | 66 | 36.293 | 25.996 | 81.686 | 1.00 | 0.00 | xxxx | 509 |
| ATOM | 510 | N | VAL | A | 67 | 32.607 | 28.264 | 79.673 | 1.00 | 0.00 | xxxx | 510 |
| ATOM | 511 | CA | VAL | A | 67 | 31.380 | 27.994 | 78.935 | 1.00 | 0.00 | xxxx | 511 |
| ATOM | 512 | C | VAL | A | 67 | 31.706 | 27.288 | 77.625 | 1.00 | 0.00 | xxxx | 512 |
| ATOM | 513 | O | VAL | A | 67 | 31.181 | 26.207 | 77.331 | 1.00 | 0.00 | xxxx | 513 |
| ATOM | 514 | CB | VAL | A | 67 | 30.587 | 29.294 | 78.713 | 1.00 | 0.00 | xxxx | 514 |
| ATOM | 515 | CG1 | VAL | A | 67 | 29.312 | 29.005 | 77.929 | 1.00 | 0.00 | xxxx | 515 |
| ATOM | 516 | CG2 | VAL | A | 67 | 30.270 | 29.937 | 80.054 | 1.00 | 0.00 | xxxx | 516 |
| ATOM | 517 | N | ASP | A | 68 | 32.586 | 27.896 | 76.825 | 1.00 | 0.00 | xxxx | 517 |
| ATOM | 518 | CA | ASP | A | 68 | 33.089 | 27.344 | 75.570 | 1.00 | 0.00 | xxxx | 518 |
| ATOM | 519 | C | ASP | A | 68 | 34.566 | 27.052 | 75.820 | 1.00 | 0.00 | xxxx | 519 |
| ATOM | 520 | O | ASP | A | 68 | 35.363 | 27.978 | 75.992 | 1.00 | 0.00 | xxxx | 520 |
| ATOM | 521 | CB | ASP | A | 68 | 32.902 | 28.403 | 74.483 | 1.00 | 0.00 | xxxx | 521 |
| ATOM | 522 | CG | ASP | A | 68 | 33.509 | 28.022 | 73.150 | 1.00 | 0.00 | xxxx | 522 |
| ATOM | 523 | OD1 | ASP | A | 68 | 34.190 | 26.984 | 73.041 | 1.00 | 0.00 | xxxx | 523 |
| ATOM | 524 | OD2 | ASP | A | 68 | 33.299 | 28.793 | 72.184 | 1.00 | 0.00 | xxxx | 524 |
| ATOM | 525 | N | ARG | A | 69 | 34.938 | 25.771 | 75.870 | 1.00 | 0.00 | xxxx | 525 |
| ATOM | 526 | CA | ARG | A | 69 | 36.314 | 25.449 | 76.240 | 1.00 | 0.00 | xxxx | 526 |
| ATOM | 527 | C | ARG | A | 69 | 37.342 | 25.935 | 75.220 | 1.00 | 0.00 | xxxx | 527 |
| ATOM | 528 | O | ARG | A | 69 | 38.520 | 26.068 | 75.573 | 1.00 | 0.00 | xxxx | 528 |
| ATOM | 529 | CB | ARG | A | 69 | 36.478 | 23.954 | 76.527 | 1.00 | 0.00 | xxxx | 529 |
| ATOM | 530 | CG | ARG | A | 69 | 36.394 | 23.077 | 75.307 | 1.00 | 0.00 | xxxx | 530 |
| ATOM | 531 | CD | ARG | A | 69 | 36.008 | 21.656 | 75.723 | 1.00 | 0.00 | xxxx | 531 |
| ATOM | 532 | NE | ARG | A | 69 | 35.941 | 20.732 | 74.600 | 1.00 | 0.00 | xxxx | 532 |
| ATOM | 533 | CZ | ARG | A | 69 | 36.959 | 19.992 | 74.168 | 1.00 | 0.00 | xxxx | 533 |
| ATOM | 534 | NH1 | ARG | A | 69 | 38.142 | 20.069 | 74.758 | 1.00 | 0.00 | xxxx | 534 |
| ATOM | 535 | NH2 | ARG | A | 69 | 36.789 | 19.184 | 73.138 | 1.00 | 0.00 | xxxx | 535 |
| ATOM | 536 | N | THR | A | 70 | 36.934 | 26.219 | 73.984 | 1.00 | 0.00 | xxxx | 536 |
| ATOM | 537 | CA | THR | A | 70 | 37.874 | 26.788 | 73.025 | 1.00 | 0.00 | xxxx | 537 |
| ATOM | 538 | C | THR | A | 70 | 38.253 | 28.221 | 73.383 | 1.00 | 0.00 | xxxx | 538 |
| ATOM | 539 | O | THR | A | 70 | 39.257 | 28.731 | 72.874 | 1.00 | 0.00 | xxxx | 539 |
| ATOM | 540 | CB | THR | A | 70 | 37.344 | 26.680 | 71.590 | 1.00 | 0.00 | xxxx | 540 |
| ATOM | 541 | OG1 | THR | A | 70 | 36.207 | 27.530 | 71.423 | 1.00 | 0.00 | xxxx | 541 |
| ATOM | 542 | CG2 | THR | A | 70 | 36.956 | 25.244 | 71.267 | 1.00 | 0.00 | xxxx | 542 |
| ATOM | 543 | N | ALA | A | 71 | 37.482 | 28.876 | 74.253 | 1.00 | 0.00 | xxxx | 543 |
| ATOM | 544 | CA | ALA | A | 71 | 37.821 | 30.216 | 74.719 | 1.00 | 0.00 | xxxx | 544 |
| ATOM | 545 | C | ALA | A | 71 | 38.947 | 30.224 | 75.737 | 1.00 | 0.00 | xxxx | 545 |
| ATOM | 546 | O | ALA | A | 71 | 39.421 | 31.308 | 76.104 | 1.00 | 0.00 | xxxx | 546 |
| ATOM | 547 | CB | ALA | A | 71 | 36.596 | 30.899 | 75.331 | 1.00 | 0.00 | xxxx | 547 |
| ATOM | 548 | N | ALA | A | 72 | 39.407 | 29.056 | 76.180 | 1.00 | 0.00 | xxxx | 548 |
| ATOM | 549 | CA | ALA | A | 72 | 40.478 | 29.019 | 77.168 | 1.00 | 0.00 | xxxx | 549 |
| ATOM | 550 | C | ALA | A | 72 | 41.765 | 29.642 | 76.638 | 1.00 | 0.00 | xxxx | 550 |
| ATOM | 551 | O | ALA | A | 72 | 42.545 | 30.201 | 77.417 | 1.00 | 0.00 | xxxx | 551 |
| ATOM | 552 | CB | ALA | A | 72 | 40.710 | 27.581 | 77.640 | 1.00 | 0.00 | xxxx | 552 |
| ATOM | 553 | N | GLY | A | 73 | 41.988 | 29.591 | 75.324 | 1.00 | 0.00 | xxxx | 553 |
| ATOM | 554 | CA | GLY | A | 73 | 43.196 | 30.180 | 74.765 | 1.00 | 0.00 | xxxx | 554 |
| ATOM | 555 | C | GLY | A | 73 | 43.308 | 31.668 | 75.049 | 1.00 | 0.00 | xxxx | 555 |
| ATOM | 556 | O | GLY | A | 73 | 44.358 | 32.156 | 75.469 | 1.00 | 0.00 | xxxx | 556 |
| ATOM | 557 | N | THR | A | 74 | 42.218 | 32.404 | 74.826 | 1.00 | 0.00 | xxxx | 557 |
| ATOM | 558 | CA | THR | A | 74 | 42.237 | 33.842 | 75.074 | 1.00 | 0.00 | xxxx | 558 |
| ATOM | 559 | C | THR | A | 74 | 42.330 | 34.142 | 76.565 | 1.00 | 0.00 | xxxx | 559 |
| ATOM | 560 | O | THR | A | 74 | 43.006 | 35.094 | 76.973 | 1.00 | 0.00 | xxxx | 560 |
| ATOM | 561 | CB | THR | A | 74 | 40.990 | 34.484 | 74.469 | 1.00 | 0.00 | xxxx | 561 |
| ATOM | 562 | OG1 | THR | A | 74 | 40.899 | 34.126 | 73.085 | 1.00 | 0.00 | xxxx | 562 |
| ATOM | 563 | CG2 | THR | A | 74 | 41.061 | 35.997 | 74.587 | 1.00 | 0.00 | xxxx | 563 |
| ATOM | 564 | N | ILE | A | 75 | 41.662 | 33.340 | 77.393 | 1.00 | 0.00 | xxxx | 564 |
| ATOM | 565 | CA | ILE | A | 75 | 41.765 | 33.525 | 78.838 | 1.00 | 0.00 | xxxx | 565 |
| ATOM | 566 | C | ILE | A | 75 | 43.202 | 33.322 | 79.304 | 1.00 | 0.00 | xxxx | 566 |
| ATOM | 567 | O | ILE | A | 75 | 43.710 | 34.083 | 80.138 | 1.00 | 0.00 | xxxx | 567 |
| ATOM | 568 | CB | ILE | A | 75 | 40.771 | 32.605 | 79.565 | 1.00 | 0.00 | xxxx | 568 |
| ATOM | 569 | CG1 | ILE | A | 75 | 39.344 | 33.021 | 79.219 | 1.00 | 0.00 | xxxx | 569 |
| ATOM | 570 | CG2 | ILE | A | 75 | 40.994 | 32.655 | 81.070 | 1.00 | 0.00 | xxxx | 570 |
| ATOM | 571 | CD1 | ILE | A | 75 | 38.305 | 32.030 | 79.674 | 1.00 | 0.00 | xxxx | 571 |
| ATOM | 572 | N | ILE | A | 76 | 43.887 | 32.309 | 78.757 | 1.00 | 0.00 | xxxx | 572 |
| ATOM | 573 | CA | ILE | A | 76 | 45.294 | 32.094 | 79.085 | 1.00 | 0.00 | xxxx | 573 |
| ATOM | 574 | C | ILE | A | 76 | 46.139 | 33.290 | 78.665 | 1.00 | 0.00 | xxxx | 574 |
| ATOM | 575 | O | ILE | A | 76 | 47.021 | 33.729 | 79.412 | 1.00 | 0.00 | xxxx | 575 |
| ATOM | 576 | CB | ILE | A | 76 | 45.804 | 30.775 | 78.476 | 1.00 | 0.00 | xxxx | 576 |
| ATOM | 577 | CG1 | ILE | A | 76 | 45.122 | 29.589 | 79.156 | 1.00 | 0.00 | xxxx | 577 |
| ATOM | 578 | CG2 | ILE | A | 76 | 47.316 | 30.664 | 78.638 | 1.00 | 0.00 | xxxx | 578 |
| ATOM | 579 | CD1 | ILE | A | 76 | 45.268 | 28.282 | 78.392 | 1.00 | 0.00 | xxxx | 579 |
| ATOM | 580 | N | ASP | A | 77 | 45.887 | 33.840 | 77.467 | 1.00 | 0.00 | xxxx | 580 |
| ATOM | 581 | CA | ASP | A | 77 | 46.636 | 35.016 | 77.025 | 1.00 | 0.00 | xxxx | 581 |
| ATOM | 582 | C | ASP | A | 77 | 46.484 | 36.159 | 78.020 | 1.00 | 0.00 | xxxx | 582 |
| ATOM | 583 | O | ASP | A | 77 | 47.464 | 36.818 | 78.387 | 1.00 | 0.00 | xxxx | 583 |
| ATOM | 584 | CB | ASP | A | 77 | 46.158 | 35.467 | 75.644 | 1.00 | 0.00 | xxxx | 584 |
| ATOM | 585 | CG | ASP | A | 77 | 46.590 | 34.533 | 74.533 | 1.00 | 0.00 | xxxx | 585 |
| ATOM | 586 | OD1 | ASP | A | 77 | 47.614 | 33.841 | 74.696 | 1.00 | 0.00 | xxxx | 586 |
| ATOM | 587 | OD2 | ASP | A | 77 | 45.904 | 34.500 | 73.489 | 1.00 | 0.00 | xxxx | 587 |
| ATOM | 588 | N | LYS | A | 78 | 45.253 | 36.401 | 78.471 | 1.00 | 0.00 | xxxx | 588 |
| ATOM | 589 | CA | LYS | A | 78 | 45.010 | 37.504 | 79.394 | 1.00 | 0.00 | xxxx | 589 |
| ATOM | 590 | C | LYS | A | 78 | 45.652 | 37.237 | 80.747 | 1.00 | 0.00 | xxxx | 590 |
| ATOM | 591 | O | LYS | A | 78 | 46.258 | 38.138 | 81.339 | 1.00 | 0.00 | xxxx | 591 |
| ATOM | 592 | CB | LYS | A | 78 | 43.507 | 37.715 | 79.564 | 1.00 | 0.00 | xxxx | 592 |
| ATOM | 593 | CG | LYS | A | 78 | 42.806 | 38.240 | 78.327 | 1.00 | 0.00 | xxxx | 593 |
| ATOM | 594 | CD | LYS | A | 78 | 41.302 | 38.175 | 78.521 | 1.00 | 0.00 | xxxx | 594 |
| ATOM | 595 | CE | LYS | A | 78 | 40.571 | 38.956 | 77.442 | 1.00 | 0.00 | xxxx | 595 |
| ATOM | 596 | NZ | LYS | A | 78 | 39.097 | 38.909 | 77.649 | 1.00 | 0.00 | xxxx | 596 |
| ATOM | 597 | N | ALA | A | 79 | 45.525 | 36.008 | 81.251 | 1.00 | 0.00 | xxxx | 597 |
| ATOM | 598 | CA | ALA | A | 79 | 46.115 | 35.671 | 82.541 | 1.00 | 0.00 | xxxx | 598 |
| ATOM | 599 | C | ALA | A | 79 | 47.635 | 35.750 | 82.490 | 1.00 | 0.00 | xxxx | 599 |
| ATOM | 600 | O | ALA | A | 79 | 48.268 | 36.274 | 83.414 | 1.00 | 0.00 | xxxx | 600 |
| ATOM | 601 | CB | ALA | A | 79 | 45.666 | 34.273 | 82.964 | 1.00 | 0.00 | xxxx | 601 |
| ATOM | 602 | N | LYS | A | 80 | 48.238 | 35.242 | 81.412 | 1.00 | 0.00 | xxxx | 602 |
| ATOM | 603 | CA | LYS | A | 80 | 49.691 | 35.320 | 81.266 | 1.00 | 0.00 | xxxx | 603 |
| ATOM | 604 | C | LYS | A | 80 | 50.169 | 36.768 | 81.250 | 1.00 | 0.00 | xxxx | 604 |
| ATOM | 605 | O | LYS | A | 80 | 51.180 | 37.105 | 81.878 | 1.00 | 0.00 | xxxx | 605 |
| ATOM | 606 | CB | LYS | A | 80 | 50.113 | 34.607 | 79.981 | 1.00 | 0.00 | xxxx | 606 |
| ATOM | 607 | CG | LYS | A | 80 | 51.616 | 34.566 | 79.750 | 1.00 | 0.00 | xxxx | 607 |
| ATOM | 608 | CD | LYS | A | 80 | 51.944 | 33.783 | 78.485 | 1.00 | 0.00 | xxxx | 608 |
| ATOM | 609 | CE | LYS | A | 80 | 53.444 | 33.672 | 78.273 | 1.00 | 0.00 | xxxx | 609 |
| ATOM | 610 | NZ | LYS | A | 80 | 53.760 | 32.800 | 77.108 | 1.00 | 0.00 | xxxx | 610 |
| ATOM | 611 | N | GLN | A | 81 | 49.453 | 37.640 | 80.537 | 1.00 | 0.00 | xxxx | 611 |
| ATOM | 612 | CA | GLN | A | 81 | 49.845 | 39.044 | 80.476 | 1.00 | 0.00 | xxxx | 612 |
| ATOM | 613 | C | GLN | A | 81 | 49.766 | 39.701 | 81.849 | 1.00 | 0.00 | xxxx | 613 |
| ATOM | 614 | O | GLN | A | 81 | 50.602 | 40.547 | 82.187 | 1.00 | 0.00 | xxxx | 614 |
| ATOM | 615 | CB | GLN | A | 81 | 48.974 | 39.782 | 79.461 | 1.00 | 0.00 | xxxx | 615 |
| ATOM | 616 | CG | GLN | A | 81 | 49.389 | 41.224 | 79.220 | 1.00 | 0.00 | xxxx | 616 |
| ATOM | 617 | CD | GLN | A | 81 | 50.816 | 41.343 | 78.709 | 1.00 | 0.00 | xxxx | 617 |
| ATOM | 618 | OE1 | GLN | A | 81 | 51.242 | 40.586 | 77.835 | 1.00 | 0.00 | xxxx | 618 |
| ATOM | 619 | NE2 | GLN | A | 81 | 51.566 | 42.293 | 79.262 | 1.00 | 0.00 | xxxx | 619 |
| ATOM | 620 | N | ALA | A | 82 | 48.780 | 39.312 | 82.660 | 1.00 | 0.00 | xxxx | 620 |
| ATOM | 621 | CA | ALA | A | 82 | 48.623 | 39.844 | 84.008 | 1.00 | 0.00 | xxxx | 621 |
| ATOM | 622 | C | ALA | A | 82 | 49.461 | 39.096 | 85.034 | 1.00 | 0.00 | xxxx | 622 |
| ATOM | 623 | O | ALA | A | 82 | 49.497 | 39.505 | 86.201 | 1.00 | 0.00 | xxxx | 623 |
| ATOM | 624 | CB | ALA | A | 82 | 47.153 | 39.754 | 84.433 | 1.00 | 0.00 | xxxx | 624 |
| ATOM | 625 | N | ASN | A | 83 | 50.124 | 38.015 | 84.625 | 1.00 | 0.00 | xxxx | 625 |
| ATOM | 626 | CA | ASN | A | 83 | 50.869 | 37.141 | 85.534 | 1.00 | 0.00 | xxxx | 626 |
| ATOM | 627 | C | ASN | A | 83 | 49.992 | 36.622 | 86.673 | 1.00 | 0.00 | xxxx | 627 |
| ATOM | 628 | O | ASN | A | 83 | 50.401 | 36.599 | 87.834 | 1.00 | 0.00 | xxxx | 628 |
| ATOM | 629 | CB | ASN | A | 83 | 52.149 | 37.799 | 86.058 | 1.00 | 0.00 | xxxx | 629 |
| ATOM | 630 | CG | ASN | A | 83 | 53.128 | 36.794 | 86.629 | 1.00 | 0.00 | xxxx | 630 |
| ATOM | 631 | OD1 | ASN | A | 83 | 53.159 | 35.632 | 86.215 | 1.00 | 0.00 | xxxx | 631 |
| ATOM | 632 | ND2 | ASN | A | 83 | 53.924 | 37.229 | 87.597 | 1.00 | 0.00 | xxxx | 632 |
| ATOM | 633 | N | ILE | A | 84 | 48.776 | 36.201 | 86.341 | 1.00 | 0.00 | xxxx | 633 |
| ATOM | 634 | CA | ILE | A | 84 | 47.852 | 35.601 | 87.298 | 1.00 | 0.00 | xxxx | 634 |
| ATOM | 635 | C | ILE | A | 84 | 47.658 | 34.144 | 86.894 | 1.00 | 0.00 | xxxx | 635 |
| ATOM | 636 | O | ILE | A | 84 | 47.107 | 33.871 | 85.815 | 1.00 | 0.00 | xxxx | 636 |
| ATOM | 637 | CB | ILE | A | 84 | 46.518 | 36.361 | 87.359 | 1.00 | 0.00 | xxxx | 637 |
| ATOM | 638 | CG1 | ILE | A | 84 | 46.762 | 37.810 | 87.791 | 1.00 | 0.00 | xxxx | 638 |
| ATOM | 639 | CG2 | ILE | A | 84 | 45.558 | 35.670 | 88.330 | 1.00 | 0.00 | xxxx | 639 |
| ATOM | 640 | CD1 | ILE | A | 84 | 45.500 | 38.666 | 87.830 | 1.00 | 0.00 | xxxx | 640 |
| ATOM | 641 | N | PRO | A | 85 | 48.091 | 33.178 | 87.706 | 1.00 | 0.00 | xxxx | 641 |
| ATOM | 642 | CA | PRO | A | 85 | 47.973 | 31.764 | 87.327 | 1.00 | 0.00 | xxxx | 642 |
| ATOM | 643 | C | PRO | A | 85 | 46.520 | 31.335 | 87.238 | 1.00 | 0.00 | xxxx | 643 |
| ATOM | 644 | O | PRO | A | 85 | 45.632 | 31.916 | 87.868 | 1.00 | 0.00 | xxxx | 644 |
| ATOM | 645 | CB | PRO | A | 85 | 48.674 | 31.022 | 88.473 | 1.00 | 0.00 | xxxx | 645 |
| ATOM | 646 | CG | PRO | A | 85 | 49.463 | 32.051 | 89.196 | 1.00 | 0.00 | xxxx | 646 |
| ATOM | 647 | CD | PRO | A | 85 | 48.742 | 33.353 | 89.015 | 1.00 | 0.00 | xxxx | 647 |
| ATOM | 648 | N | VAL | A | 86 | 46.274 | 30.286 | 86.454 | 1.00 | 0.00 | xxxx | 648 |
| ATOM | 649 | CA | VAL | A | 86 | 44.901 | 29.859 | 86.196 | 1.00 | 0.00 | xxxx | 649 |
| ATOM | 650 | C | VAL | A | 86 | 44.797 | 28.340 | 86.202 | 1.00 | 0.00 | xxxx | 650 |
| ATOM | 651 | O | VAL | A | 86 | 45.611 | 27.646 | 85.583 | 1.00 | 0.00 | xxxx | 651 |
| ATOM | 652 | CB | VAL | A | 86 | 44.352 | 30.464 | 84.890 | 1.00 | 0.00 | xxxx | 652 |
| ATOM | 653 | CG1 | VAL | A | 86 | 45.289 | 30.182 | 83.719 | 1.00 | 0.00 | xxxx | 653 |
| ATOM | 654 | CG2 | VAL | A | 86 | 42.942 | 29.968 | 84.607 | 1.00 | 0.00 | xxxx | 654 |
| ATOM | 655 | N | VAL | A | 87 | 43.791 | 27.823 | 86.898 | 1.00 | 0.00 | xxxx | 655 |
| ATOM | 656 | CA | VAL | A | 87 | 43.403 | 26.420 | 86.794 | 1.00 | 0.00 | xxxx | 656 |
| ATOM | 657 | C | VAL | A | 87 | 42.027 | 26.354 | 86.148 | 1.00 | 0.00 | xxxx | 657 |
| ATOM | 658 | O | VAL | A | 87 | 41.089 | 27.044 | 86.577 | 1.00 | 0.00 | xxxx | 658 |
| ATOM | 659 | CB | VAL | A | 87 | 43.467 | 25.691 | 88.150 | 1.00 | 0.00 | xxxx | 659 |
| ATOM | 660 | CG1 | VAL | A | 87 | 42.648 | 26.394 | 89.236 | 1.00 | 0.00 | xxxx | 660 |
| ATOM | 661 | CG2 | VAL | A | 87 | 43.054 | 24.220 | 87.999 | 1.00 | 0.00 | xxxx | 661 |
| ATOM | 662 | N | PHE | A | 88 | 41.928 | 25.582 | 85.072 | 1.00 | 0.00 | xxxx | 662 |
| ATOM | 663 | CA | PHE | A | 88 | 40.648 | 25.303 | 84.448 | 1.00 | 0.00 | xxxx | 663 |
| ATOM | 664 | C | PHE | A | 88 | 40.078 | 24.014 | 85.014 | 1.00 | 0.00 | xxxx | 664 |
| ATOM | 665 | O | PHE | A | 88 | 40.818 | 23.151 | 85.499 | 1.00 | 0.00 | xxxx | 665 |
| ATOM | 666 | CB | PHE | A | 88 | 40.792 | 25.194 | 82.930 | 1.00 | 0.00 | xxxx | 666 |
| ATOM | 667 | CG | PHE | A | 88 | 41.186 | 26.487 | 82.271 | 1.00 | 0.00 | xxxx | 667 |
| ATOM | 668 | CD1 | PHE | A | 88 | 42.515 | 26.781 | 82.032 | 1.00 | 0.00 | xxxx | 668 |
| ATOM | 669 | CD2 | PHE | A | 88 | 40.219 | 27.411 | 81.928 | 1.00 | 0.00 | xxxx | 669 |
| ATOM | 670 | CE1 | PHE | A | 88 | 42.878 | 27.966 | 81.428 | 1.00 | 0.00 | xxxx | 670 |
| ATOM | 671 | CE2 | PHE | A | 88 | 40.578 | 28.605 | 81.324 | 1.00 | 0.00 | xxxx | 671 |
| ATOM | 672 | CZ | PHE | A | 88 | 41.913 | 28.873 | 81.079 | 1.00 | 0.00 | xxxx | 672 |
| ATOM | 673 | N | PHE | A | 89 | 38.754 | 23.890 | 84.964 | 1.00 | 0.00 | xxxx | 673 |
| ATOM | 674 | CA | PHE | A | 89 | 38.138 | 22.676 | 85.482 | 1.00 | 0.00 | xxxx | 674 |
| ATOM | 675 | C | PHE | A | 89 | 36.867 | 22.330 | 84.727 | 1.00 | 0.00 | xxxx | 675 |
| ATOM | 676 | O | PHE | A | 89 | 36.149 | 23.222 | 84.267 | 1.00 | 0.00 | xxxx | 676 |
| ATOM | 677 | CB | PHE | A | 89 | 37.961 | 22.673 | 87.012 | 1.00 | 0.00 | xxxx | 677 |
| ATOM | 678 | CG | PHE | A | 89 | 37.089 | 23.782 | 87.578 | 1.00 | 0.00 | xxxx | 678 |
| ATOM | 679 | CD1 | PHE | A | 89 | 35.896 | 23.470 | 88.214 | 1.00 | 0.00 | xxxx | 679 |
| ATOM | 680 | CD2 | PHE | A | 89 | 37.503 | 25.112 | 87.565 | 1.00 | 0.00 | xxxx | 680 |
| ATOM | 681 | CE1 | PHE | A | 89 | 35.109 | 24.467 | 88.797 | 1.00 | 0.00 | xxxx | 681 |
| ATOM | 682 | CE2 | PHE | A | 89 | 36.706 | 26.122 | 88.140 | 1.00 | 0.00 | xxxx | 682 |
| ATOM | 683 | CZ | PHE | A | 89 | 35.519 | 25.786 | 88.760 | 1.00 | 0.00 | xxxx | 683 |
| ATOM | 684 | N | ASN | A | 90 | 36.640 | 21.017 | 84.573 | 1.00 | 0.00 | xxxx | 684 |
| ATOM | 685 | CA | ASN | A | 90 | 35.397 | 20.414 | 84.098 | 1.00 | 0.00 | xxxx | 685 |
| ATOM | 686 | C | ASN | A | 90 | 35.146 | 20.491 | 82.586 | 1.00 | 0.00 | xxxx | 686 |
| ATOM | 687 | O | ASN | A | 90 | 34.669 | 19.517 | 81.999 | 1.00 | 0.00 | xxxx | 687 |
| ATOM | 688 | CB | ASN | A | 90 | 34.186 | 20.871 | 84.920 | 1.00 | 0.00 | xxxx | 688 |
| ATOM | 689 | CG | ASN | A | 90 | 32.872 | 20.498 | 84.265 | 1.00 | 0.00 | xxxx | 689 |
| ATOM | 690 | OD1 | ASN | A | 90 | 32.284 | 21.310 | 83.565 | 1.00 | 0.00 | xxxx | 690 |
| ATOM | 691 | ND2 | ASN | A | 90 | 32.415 | 19.264 | 84.478 | 1.00 | 0.00 | xxxx | 691 |
| ATOM | 692 | N | LYS | A | 91 | 35.426 | 21.627 | 81.945 | 1.00 | 0.00 | xxxx | 692 |
| ATOM | 693 | CA | LYS | A | 91 | 35.463 | 21.697 | 80.485 | 1.00 | 0.00 | xxxx | 693 |
| ATOM | 694 | C | LYS | A | 91 | 36.912 | 21.923 | 80.081 | 1.00 | 0.00 | xxxx | 694 |
| ATOM | 695 | O | LYS | A | 91 | 37.547 | 22.880 | 80.539 | 1.00 | 0.00 | xxxx | 695 |
| ATOM | 696 | CB | LYS | A | 91 | 34.549 | 22.803 | 79.949 | 1.00 | 0.00 | xxxx | 696 |
| ATOM | 697 | CG | LYS | A | 91 | 33.064 | 22.644 | 80.345 | 1.00 | 0.00 | xxxx | 697 |
| ATOM | 698 | CD | LYS | A | 91 | 32.142 | 23.327 | 79.326 | 1.00 | 0.00 | xxxx | 698 |
| ATOM | 699 | CE | LYS | A | 91 | 30.781 | 23.654 | 79.916 | 1.00 | 0.00 | xxxx | 699 |
| ATOM | 700 | NZ | LYS | A | 91 | 29.882 | 24.261 | 78.901 | 1.00 | 0.00 | xxxx | 700 |
| ATOM | 701 | N | GLU | A | 92 | 37.437 | 21.027 | 79.254 | 1.00 | 0.00 | xxxx | 701 |
| ATOM | 702 | CA | GLU | A | 92 | 38.879 | 20.872 | 79.104 | 1.00 | 0.00 | xxxx | 702 |
| ATOM | 703 | C | GLU | A | 92 | 39.423 | 21.794 | 78.015 | 1.00 | 0.00 | xxxx | 703 |
| ATOM | 704 | O | GLU | A | 92 | 39.003 | 21.685 | 76.856 | 1.00 | 0.00 | xxxx | 704 |
| ATOM | 705 | CB | GLU | A | 92 | 39.200 | 19.429 | 78.753 | 1.00 | 0.00 | xxxx | 705 |
| ATOM | 706 | CG | GLU | A | 92 | 40.684 | 19.125 | 78.709 | 1.00 | 0.00 | xxxx | 706 |
| ATOM | 707 | CD | GLU | A | 92 | 40.980 | 17.677 | 78.380 | 1.00 | 0.00 | xxxx | 707 |
| ATOM | 708 | OE1 | GLU | A | 92 | 40.090 | 16.814 | 78.537 | 1.00 | 0.00 | xxxx | 708 |
| ATOM | 709 | OE2 | GLU | A | 92 | 42.114 | 17.396 | 77.934 | 1.00 | 0.00 | xxxx | 709 |
| ATOM | 710 | N | PRO | A | 93 | 40.368 | 22.684 | 78.324 | 1.00 | 0.00 | xxxx | 710 |
| ATOM | 711 | CA | PRO | A | 93 | 41.034 | 23.444 | 77.259 | 1.00 | 0.00 | xxxx | 711 |
| ATOM | 712 | C | PRO | A | 93 | 41.689 | 22.509 | 76.256 | 1.00 | 0.00 | xxxx | 712 |
| ATOM | 713 | O | PRO | A | 93 | 42.107 | 21.392 | 76.583 | 1.00 | 0.00 | xxxx | 713 |
| ATOM | 714 | CB | PRO | A | 93 | 42.093 | 24.257 | 78.012 | 1.00 | 0.00 | xxxx | 714 |
| ATOM | 715 | CG | PRO | A | 93 | 41.509 | 24.413 | 79.383 | 1.00 | 0.00 | xxxx | 715 |
| ATOM | 716 | CD | PRO | A | 93 | 40.824 | 23.096 | 79.663 | 1.00 | 0.00 | xxxx | 716 |
| ATOM | 717 | N | LEU | A | 94 | 41.800 | 22.993 | 75.024 | 1.00 | 0.00 | xxxx | 717 |
| ATOM | 718 | CA | LEU | A | 94 | 42.433 | 22.213 | 73.972 | 1.00 | 0.00 | xxxx | 718 |
| ATOM | 719 | C | LEU | A | 94 | 43.893 | 21.933 | 74.330 | 1.00 | 0.00 | xxxx | 719 |
| ATOM | 720 | O | LEU | A | 94 | 44.551 | 22.767 | 74.965 | 1.00 | 0.00 | xxxx | 720 |
| ATOM | 721 | CB | LEU | A | 94 | 42.347 | 22.978 | 72.650 | 1.00 | 0.00 | xxxx | 721 |
| ATOM | 722 | CG | LEU | A | 94 | 40.914 | 23.234 | 72.166 | 1.00 | 0.00 | xxxx | 722 |
| ATOM | 723 | CD1 | LEU | A | 94 | 40.921 | 24.250 | 71.041 | 1.00 | 0.00 | xxxx | 723 |
| ATOM | 724 | CD2 | LEU | A | 94 | 40.254 | 21.938 | 71.720 | 1.00 | 0.00 | xxxx | 724 |
| ATOM | 725 | N | PRO | A | 95 | 44.429 | 20.766 | 73.960 | 1.00 | 0.00 | xxxx | 725 |
| ATOM | 726 | CA | PRO | A | 95 | 45.799 | 20.416 | 74.381 | 1.00 | 0.00 | xxxx | 726 |
| ATOM | 727 | C | PRO | A | 95 | 46.854 | 21.452 | 74.020 | 1.00 | 0.00 | xxxx | 727 |
| ATOM | 728 | O | PRO | A | 95 | 47.771 | 21.701 | 74.817 | 1.00 | 0.00 | xxxx | 728 |
| ATOM | 729 | CB | PRO | A | 95 | 46.057 | 19.076 | 73.674 | 1.00 | 0.00 | xxxx | 729 |
| ATOM | 730 | CG | PRO | A | 95 | 44.717 | 18.514 | 73.408 | 1.00 | 0.00 | xxxx | 730 |
| ATOM | 731 | CD | PRO | A | 95 | 43.761 | 19.661 | 73.254 | 1.00 | 0.00 | xxxx | 731 |
| ATOM | 732 | N | GLU | A | 96 | 46.771 | 22.046 | 72.830 | 1.00 | 0.00 | xxxx | 732 |
| ATOM | 733 | CA | GLU | A | 96 | 47.793 | 23.013 | 72.448 | 1.00 | 0.00 | xxxx | 733 |
| ATOM | 734 | C | GLU | A | 96 | 47.633 | 24.324 | 73.203 | 1.00 | 0.00 | xxxx | 734 |
| ATOM | 735 | O | GLU | A | 96 | 48.621 | 25.037 | 73.416 | 1.00 | 0.00 | xxxx | 735 |
| ATOM | 736 | CB | GLU | A | 96 | 47.803 | 23.232 | 70.934 | 1.00 | 0.00 | xxxx | 736 |
| ATOM | 737 | CG | GLU | A | 96 | 48.235 | 22.015 | 70.147 | 1.00 | 0.00 | xxxx | 737 |
| ATOM | 738 | CD | GLU | A | 96 | 49.629 | 21.547 | 70.513 | 1.00 | 0.00 | xxxx | 738 |
| ATOM | 739 | OE1 | GLU | A | 96 | 50.534 | 22.401 | 70.614 | 1.00 | 0.00 | xxxx | 739 |
| ATOM | 740 | OE2 | GLU | A | 96 | 49.815 | 20.327 | 70.706 | 1.00 | 0.00 | xxxx | 740 |
| ATOM | 741 | N | ASP | A | 97 | 46.412 | 24.657 | 73.623 | 1.00 | 0.00 | xxxx | 741 |
| ATOM | 742 | CA | ASP | A | 97 | 46.227 | 25.848 | 74.446 | 1.00 | 0.00 | xxxx | 742 |
| ATOM | 743 | C | ASP | A | 97 | 46.855 | 25.669 | 75.824 | 1.00 | 0.00 | xxxx | 743 |
| ATOM | 744 | O | ASP | A | 97 | 47.407 | 26.621 | 76.384 | 1.00 | 0.00 | xxxx | 744 |
| ATOM | 745 | CB | ASP | A | 97 | 44.739 | 26.187 | 74.565 | 1.00 | 0.00 | xxxx | 745 |
| ATOM | 746 | CG | ASP | A | 97 | 44.180 | 26.845 | 73.303 | 1.00 | 0.00 | xxxx | 746 |
| ATOM | 747 | OD1 | ASP | A | 97 | 44.968 | 27.227 | 72.401 | 1.00 | 0.00 | xxxx | 747 |
| ATOM | 748 | OD2 | ASP | A | 97 | 42.939 | 27.003 | 73.227 | 1.00 | 0.00 | xxxx | 748 |
| ATOM | 749 | N | MET | A | 98 | 46.809 | 24.450 | 76.372 | 1.00 | 0.00 | xxxx | 749 |
| ATOM | 750 | CA | MET | A | 98 | 47.442 | 24.187 | 77.662 | 1.00 | 0.00 | xxxx | 750 |
| ATOM | 751 | C | MET | A | 98 | 48.953 | 24.408 | 77.626 | 1.00 | 0.00 | xxxx | 751 |
| ATOM | 752 | O | MET | A | 98 | 49.562 | 24.632 | 78.679 | 1.00 | 0.00 | xxxx | 752 |
| ATOM | 753 | CB | MET | A | 98 | 47.125 | 22.758 | 78.114 | 1.00 | 0.00 | xxxx | 753 |
| ATOM | 754 | CG | MET | A | 98 | 45.647 | 22.489 | 78.418 | 1.00 | 0.00 | xxxx | 754 |
| ATOM | 755 | SD | MET | A | 98 | 45.085 | 23.366 | 79.897 | 1.00 | 0.00 | xxxx | 755 |
| ATOM | 756 | CE | MET | A | 98 | 46.117 | 22.612 | 81.168 | 1.00 | 0.00 | xxxx | 756 |
| ATOM | 757 | N | LYS | A | 99 | 49.570 | 24.361 | 76.449 | 1.00 | 0.00 | xxxx | 757 |
| ATOM | 758 | CA | LYS | A | 99 | 51.007 | 24.541 | 76.305 | 1.00 | 0.00 | xxxx | 758 |
| ATOM | 759 | C | LYS | A | 99 | 51.424 | 26.004 | 76.175 | 1.00 | 0.00 | xxxx | 759 |
| ATOM | 760 | O | LYS | A | 99 | 52.622 | 26.282 | 76.069 | 1.00 | 0.00 | xxxx | 760 |
| ATOM | 761 | CB | LYS | A | 99 | 51.500 | 23.752 | 75.092 | 1.00 | 0.00 | xxxx | 761 |
| ATOM | 762 | CG | LYS | A | 99 | 51.309 | 22.254 | 75.227 | 1.00 | 0.00 | xxxx | 762 |
| ATOM | 763 | CD | LYS | A | 99 | 51.695 | 21.541 | 73.938 | 1.00 | 0.00 | xxxx | 763 |
| ATOM | 764 | CE | LYS | A | 99 | 51.555 | 20.033 | 74.071 | 1.00 | 0.00 | xxxx | 764 |
| ATOM | 765 | NZ | LYS | A | 99 | 52.567 | 19.465 | 75.004 | 1.00 | 0.00 | xxxx | 765 |
| ATOM | 766 | N | LYS | A | 100 | 50.472 | 26.942 | 76.211 | 1.00 | 0.00 | xxxx | 766 |
| ATOM | 767 | CA | LYS | A | 100 | 50.794 | 28.352 | 76.012 | 1.00 | 0.00 | xxxx | 767 |
| ATOM | 768 | C | LYS | A | 100 | 51.533 | 28.962 | 77.197 | 1.00 | 0.00 | xxxx | 768 |
| ATOM | 769 | O | LYS | A | 100 | 52.212 | 29.977 | 77.025 | 1.00 | 0.00 | xxxx | 769 |
| ATOM | 770 | CB | LYS | A | 100 | 49.513 | 29.152 | 75.759 | 1.00 | 0.00 | xxxx | 770 |
| ATOM | 771 | CG | LYS | A | 100 | 48.831 | 28.827 | 74.448 | 1.00 | 0.00 | xxxx | 771 |
| ATOM | 772 | CD | LYS | A | 100 | 47.377 | 29.291 | 74.441 | 1.00 | 0.00 | xxxx | 772 |
| ATOM | 773 | CE | LYS | A | 100 | 47.258 | 30.796 | 74.398 | 1.00 | 0.00 | xxxx | 773 |
| ATOM | 774 | NZ | LYS | A | 100 | 47.635 | 31.353 | 73.062 | 1.00 | 0.00 | xxxx | 774 |
| ATOM | 775 | N | TRP | A | 101 | 51.406 | 28.382 | 78.391 | 1.00 | 0.00 | xxxx | 775 |
| ATOM | 776 | CA | TRP | A | 101 | 52.010 | 28.966 | 79.579 | 1.00 | 0.00 | xxxx | 776 |
| ATOM | 777 | C | TRP | A | 101 | 52.277 | 27.853 | 80.580 | 1.00 | 0.00 | xxxx | 777 |
| ATOM | 778 | O | TRP | A | 101 | 51.626 | 26.806 | 80.555 | 1.00 | 0.00 | xxxx | 778 |
| ATOM | 779 | CB | TRP | A | 101 | 51.056 | 29.999 | 80.188 | 1.00 | 0.00 | xxxx | 779 |
| ATOM | 780 | CG | TRP | A | 101 | 51.640 | 30.945 | 81.199 | 1.00 | 0.00 | xxxx | 780 |
| ATOM | 781 | CD1 | TRP | A | 101 | 52.949 | 31.320 | 81.335 | 1.00 | 0.00 | xxxx | 781 |
| ATOM | 782 | CD2 | TRP | A | 101 | 50.912 | 31.647 | 82.215 | 1.00 | 0.00 | xxxx | 782 |
| ATOM | 783 | NE1 | TRP | A | 101 | 53.078 | 32.218 | 82.377 | 1.00 | 0.00 | xxxx | 783 |
| ATOM | 784 | CE2 | TRP | A | 101 | 51.842 | 32.434 | 82.930 | 1.00 | 0.00 | xxxx | 784 |
| ATOM | 785 | CE3 | TRP | A | 101 | 49.561 | 31.694 | 82.582 | 1.00 | 0.00 | xxxx | 785 |
| ATOM | 786 | CZ2 | TRP | A | 101 | 51.464 | 33.257 | 83.995 | 1.00 | 0.00 | xxxx | 786 |
| ATOM | 787 | CZ3 | TRP | A | 101 | 49.188 | 32.507 | 83.645 | 1.00 | 0.00 | xxxx | 787 |
| ATOM | 788 | CH2 | TRP | A | 101 | 50.136 | 33.277 | 84.336 | 1.00 | 0.00 | xxxx | 788 |
| ATOM | 789 | N | ASP | A | 102 | 53.224 | 28.102 | 81.483 | 1.00 | 0.00 | xxxx | 789 |
| ATOM | 790 | CA | ASP | A | 102 | 53.626 | 27.126 | 82.488 | 1.00 | 0.00 | xxxx | 790 |
| ATOM | 791 | C | ASP | A | 102 | 52.973 | 27.364 | 83.847 | 1.00 | 0.00 | xxxx | 791 |
| ATOM | 792 | O | ASP | A | 102 | 53.411 | 26.777 | 84.842 | 1.00 | 0.00 | xxxx | 792 |
| ATOM | 793 | CB | ASP | A | 102 | 55.155 | 27.039 | 82.598 | 1.00 | 0.00 | xxxx | 793 |
| ATOM | 794 | CG | ASP | A | 102 | 55.797 | 28.340 | 83.059 | 1.00 | 0.00 | xxxx | 794 |
| ATOM | 795 | OD1 | ASP | A | 102 | 55.124 | 29.388 | 83.125 | 1.00 | 0.00 | xxxx | 795 |
| ATOM | 796 | OD2 | ASP | A | 102 | 57.013 | 28.310 | 83.351 | 1.00 | 0.00 | xxxx | 796 |
| ATOM | 797 | N | LYS | A | 103 | 51.950 | 28.211 | 83.912 | 1.00 | 0.00 | xxxx | 797 |
| ATOM | 798 | CA | LYS | A | 103 | 51.156 | 28.402 | 85.119 | 1.00 | 0.00 | xxxx | 798 |
| ATOM | 799 | C | LYS | A | 103 | 49.689 | 28.099 | 84.858 | 1.00 | 0.00 | xxxx | 799 |
| ATOM | 800 | O | LYS | A | 103 | 48.798 | 28.736 | 85.427 | 1.00 | 0.00 | xxxx | 800 |
| ATOM | 801 | CB | LYS | A | 103 | 51.366 | 29.804 | 85.695 | 1.00 | 0.00 | xxxx | 801 |
| ATOM | 802 | CG | LYS | A | 103 | 52.824 | 30.084 | 86.062 | 1.00 | 0.00 | xxxx | 802 |
| ATOM | 803 | CD | LYS | A | 103 | 52.960 | 31.395 | 86.808 | 1.00 | 0.00 | xxxx | 803 |
| ATOM | 804 | CE | LYS | A | 103 | 54.412 | 31.709 | 87.110 | 1.00 | 0.00 | xxxx | 804 |
| ATOM | 805 | NZ | LYS | A | 103 | 54.516 | 32.987 | 87.861 | 1.00 | 0.00 | xxxx | 805 |
| ATOM | 806 | N | VAL | A | 104 | 49.422 | 27.112 | 83.996 | 1.00 | 0.00 | xxxx | 806 |
| ATOM | 807 | CA | VAL | A | 104 | 48.073 | 26.720 | 83.609 | 1.00 | 0.00 | xxxx | 807 |
| ATOM | 808 | C | VAL | A | 104 | 47.869 | 25.249 | 83.945 | 1.00 | 0.00 | xxxx | 808 |
| ATOM | 809 | O | VAL | A | 104 | 48.705 | 24.411 | 83.596 | 1.00 | 0.00 | xxxx | 809 |
| ATOM | 810 | CB | VAL | A | 104 | 47.826 | 26.956 | 82.111 | 1.00 | 0.00 | xxxx | 810 |
| ATOM | 811 | CG1 | VAL | A | 104 | 46.404 | 26.572 | 81.757 | 1.00 | 0.00 | xxxx | 811 |
| ATOM | 812 | CG2 | VAL | A | 104 | 48.111 | 28.418 | 81.741 | 1.00 | 0.00 | xxxx | 812 |
| ATOM | 813 | N | TYR | A | 105 | 46.758 | 24.943 | 84.611 | 1.00 | 0.00 | xxxx | 813 |
| ATOM | 814 | CA | TYR | A | 105 | 46.428 | 23.587 | 85.034 | 1.00 | 0.00 | xxxx | 814 |
| ATOM | 815 | C | TYR | A | 105 | 45.001 | 23.257 | 84.625 | 1.00 | 0.00 | xxxx | 815 |
| ATOM | 816 | O | TYR | A | 105 | 44.203 | 24.138 | 84.306 | 1.00 | 0.00 | xxxx | 816 |
| ATOM | 817 | CB | TYR | A | 105 | 46.590 | 23.433 | 86.558 | 1.00 | 0.00 | xxxx | 817 |
| ATOM | 818 | CG | TYR | A | 105 | 48.029 | 23.536 | 86.975 | 1.00 | 0.00 | xxxx | 818 |
| ATOM | 819 | CD1 | TYR | A | 105 | 48.641 | 24.778 | 87.146 | 1.00 | 0.00 | xxxx | 819 |
| ATOM | 820 | CD2 | TYR | A | 105 | 48.805 | 22.395 | 87.140 | 1.00 | 0.00 | xxxx | 820 |
| ATOM | 821 | CE1 | TYR | A | 105 | 49.989 | 24.873 | 87.485 | 1.00 | 0.00 | xxxx | 821 |
| ATOM | 822 | CE2 | TYR | A | 105 | 50.146 | 22.478 | 87.485 | 1.00 | 0.00 | xxxx | 822 |
| ATOM | 823 | CZ | TYR | A | 105 | 50.737 | 23.719 | 87.661 | 1.00 | 0.00 | xxxx | 823 |
| ATOM | 824 | OH | TYR | A | 105 | 52.074 | 23.794 | 87.999 | 1.00 | 0.00 | xxxx | 824 |
| ATOM | 825 | N | TYR | A | 106 | 44.671 | 21.959 | 84.666 | 1.00 | 0.00 | xxxx | 825 |
| ATOM | 826 | CA | TYR | A | 106 | 43.319 | 21.482 | 84.412 | 1.00 | 0.00 | xxxx | 826 |
| ATOM | 827 | C | TYR | A | 106 | 42.980 | 20.374 | 85.400 | 1.00 | 0.00 | xxxx | 827 |
| ATOM | 828 | O | TYR | A | 106 | 43.782 | 19.459 | 85.601 | 1.00 | 0.00 | xxxx | 828 |
| ATOM | 829 | CB | TYR | A | 106 | 43.141 | 20.937 | 82.973 | 1.00 | 0.00 | xxxx | 829 |
| ATOM | 830 | CG | TYR | A | 106 | 41.739 | 20.422 | 82.780 | 1.00 | 0.00 | xxxx | 830 |
| ATOM | 831 | CD1 | TYR | A | 106 | 40.675 | 21.304 | 82.676 | 1.00 | 0.00 | xxxx | 831 |
| ATOM | 832 | CD2 | TYR | A | 106 | 41.455 | 19.050 | 82.778 | 1.00 | 0.00 | xxxx | 832 |
| ATOM | 833 | CE1 | TYR | A | 106 | 39.369 | 20.857 | 82.542 | 1.00 | 0.00 | xxxx | 833 |
| ATOM | 834 | CE2 | TYR | A | 106 | 40.142 | 18.593 | 82.648 | 1.00 | 0.00 | xxxx | 834 |
| ATOM | 835 | CZ | TYR | A | 106 | 39.107 | 19.497 | 82.522 | 1.00 | 0.00 | xxxx | 835 |
| ATOM | 836 | OH | TYR | A | 106 | 37.803 | 19.075 | 82.396 | 1.00 | 0.00 | xxxx | 836 |
| ATOM | 837 | N | VAL | A | 107 | 41.773 | 20.428 | 85.968 | 1.00 | 0.00 | xxxx | 837 |
| ATOM | 838 | CA | VAL | A | 107 | 41.248 | 19.379 | 86.842 | 1.00 | 0.00 | xxxx | 838 |
| ATOM | 839 | C | VAL | A | 107 | 39.975 | 18.819 | 86.226 | 1.00 | 0.00 | xxxx | 839 |
| ATOM | 840 | O | VAL | A | 107 | 39.050 | 19.574 | 85.900 | 1.00 | 0.00 | xxxx | 840 |
| ATOM | 841 | CB | VAL | A | 107 | 40.951 | 19.903 | 88.259 | 1.00 | 0.00 | xxxx | 841 |
| ATOM | 842 | CG1 | VAL | A | 107 | 40.361 | 18.800 | 89.114 | 1.00 | 0.00 | xxxx | 842 |
| ATOM | 843 | CG2 | VAL | A | 107 | 42.213 | 20.446 | 88.908 | 1.00 | 0.00 | xxxx | 843 |
| ATOM | 844 | N | GLY | A | 108 | 39.915 | 17.493 | 86.100 | 1.00 | 0.00 | xxxx | 844 |
| ATOM | 845 | CA | GLY | A | 108 | 38.702 | 16.866 | 85.622 | 1.00 | 0.00 | xxxx | 845 |
| ATOM | 846 | C | GLY | A | 108 | 38.800 | 15.365 | 85.755 | 1.00 | 0.00 | xxxx | 846 |
| ATOM | 847 | O | GLY | A | 108 | 39.295 | 14.851 | 86.763 | 1.00 | 0.00 | xxxx | 847 |
| ATOM | 848 | N | ALA | A | 109 | 38.332 | 14.650 | 84.743 | 1.00 | 0.00 | xxxx | 848 |
| ATOM | 849 | CA | ALA | A | 109 | 38.287 | 13.200 | 84.787 | 1.00 | 0.00 | xxxx | 849 |
| ATOM | 850 | C | ALA | A | 109 | 38.364 | 12.689 | 83.362 | 1.00 | 0.00 | xxxx | 850 |
| ATOM | 851 | O | ALA | A | 109 | 38.064 | 13.415 | 82.414 | 1.00 | 0.00 | xxxx | 851 |
| ATOM | 852 | CB | ALA | A | 109 | 37.007 | 12.726 | 85.481 | 1.00 | 0.00 | xxxx | 852 |
| ATOM | 853 | N | LYS | A | 110 | 38.779 | 11.435 | 83.217 | 1.00 | 0.00 | xxxx | 853 |
| ATOM | 854 | CA | LYS | A | 110 | 38.965 | 10.850 | 81.892 | 1.00 | 0.00 | xxxx | 854 |
| ATOM | 855 | C | LYS | A | 110 | 37.616 | 10.372 | 81.379 | 1.00 | 0.00 | xxxx | 855 |
| ATOM | 856 | O | LYS | A | 110 | 37.135 | 9.308 | 81.764 | 1.00 | 0.00 | xxxx | 856 |
| ATOM | 857 | CB | LYS | A | 110 | 39.942 | 9.684 | 81.966 | 1.00 | 0.00 | xxxx | 857 |
| ATOM | 858 | CG | LYS | A | 110 | 41.382 | 10.111 | 82.235 | 1.00 | 0.00 | xxxx | 858 |
| ATOM | 859 | CD | LYS | A | 110 | 41.886 | 11.014 | 81.116 | 1.00 | 0.00 | xxxx | 859 |
| ATOM | 860 | CE | LYS | A | 110 | 43.406 | 11.034 | 81.063 | 1.00 | 0.00 | xxxx | 860 |
| ATOM | 861 | NZ | LYS | A | 110 | 43.975 | 9.730 | 80.625 | 1.00 | 0.00 | xxxx | 861 |
| ATOM | 862 | N | ALA | A | 111 | 37.021 | 11.140 | 80.467 | 1.00 | 0.00 | xxxx | 862 |
| ATOM | 863 | CA | ALA | A | 111 | 35.667 | 10.836 | 80.021 | 1.00 | 0.00 | xxxx | 863 |
| ATOM | 864 | C | ALA | A | 111 | 35.566 | 9.462 | 79.362 | 1.00 | 0.00 | xxxx | 864 |
| ATOM | 865 | O | ALA | A | 111 | 34.507 | 8.820 | 79.423 | 1.00 | 0.00 | xxxx | 865 |
| ATOM | 866 | CB | ALA | A | 111 | 35.170 | 11.933 | 79.076 | 1.00 | 0.00 | xxxx | 866 |
| ATOM | 867 | N | GLU | A | 112 | 36.657 | 9.000 | 78.744 | 1.00 | 0.00 | xxxx | 867 |
| ATOM | 868 | CA | GLU | A | 112 | 36.709 | 7.645 | 78.202 | 1.00 | 0.00 | xxxx | 868 |
| ATOM | 869 | C | GLU | A | 112 | 36.330 | 6.615 | 79.255 | 1.00 | 0.00 | xxxx | 869 |
| ATOM | 870 | O | GLU | A | 112 | 35.572 | 5.675 | 78.978 | 1.00 | 0.00 | xxxx | 870 |
| ATOM | 871 | CB | GLU | A | 112 | 38.121 | 7.355 | 77.686 | 1.00 | 0.00 | xxxx | 871 |
| ATOM | 872 | CG | GLU | A | 112 | 38.618 | 8.313 | 76.606 | 1.00 | 0.00 | xxxx | 872 |
| ATOM | 873 | CD | GLU | A | 112 | 39.455 | 9.466 | 77.141 | 1.00 | 0.00 | xxxx | 873 |
| ATOM | 874 | OE1 | GLU | A | 112 | 39.153 | 10.008 | 78.232 | 1.00 | 0.00 | xxxx | 874 |
| ATOM | 875 | OE2 | GLU | A | 112 | 40.432 | 9.841 | 76.453 | 1.00 | 0.00 | xxxx | 875 |
| ATOM | 876 | N | GLN | A | 113 | 36.855 | 6.776 | 80.473 | 1.00 | 0.00 | xxxx | 876 |
| ATOM | 877 | CA | GLN | A | 113 | 36.564 | 5.823 | 81.540 | 1.00 | 0.00 | xxxx | 877 |
| ATOM | 878 | C | GLN | A | 113 | 35.081 | 5.814 | 81.880 | 1.00 | 0.00 | xxxx | 878 |
| ATOM | 879 | O | GLN | A | 113 | 34.488 | 4.748 | 82.097 | 1.00 | 0.00 | xxxx | 879 |
| ATOM | 880 | CB | GLN | A | 113 | 37.402 | 6.166 | 82.776 | 1.00 | 0.00 | xxxx | 880 |
| ATOM | 881 | CG | GLN | A | 113 | 37.058 | 5.368 | 84.034 | 1.00 | 0.00 | xxxx | 881 |
| ATOM | 882 | CD | GLN | A | 113 | 37.890 | 5.767 | 85.260 | 1.00 | 0.00 | xxxx | 882 |
| ATOM | 883 | OE1 | GLN | A | 113 | 38.035 | 6.954 | 85.590 | 1.00 | 0.00 | xxxx | 883 |
| ATOM | 884 | NE2 | GLN | A | 113 | 38.439 | 4.765 | 85.941 | 1.00 | 0.00 | xxxx | 884 |
| ATOM | 885 | N | SER | A | 114 | 34.455 | 6.990 | 81.911 | 1.00 | 0.00 | xxxx | 885 |
| ATOM | 886 | CA | SER | A | 114 | 33.031 | 7.032 | 82.216 | 1.00 | 0.00 | xxxx | 886 |
| ATOM | 887 | C | SER | A | 114 | 32.215 | 6.359 | 81.116 | 1.00 | 0.00 | xxxx | 887 |
| ATOM | 888 | O | SER | A | 114 | 31.241 | 5.660 | 81.410 | 1.00 | 0.00 | xxxx | 888 |
| ATOM | 889 | CB | SER | A | 114 | 32.564 | 8.459 | 82.527 | 1.00 | 0.00 | xxxx | 889 |
| ATOM | 890 | OG | SER | A | 114 | 32.609 | 9.317 | 81.402 | 1.00 | 0.00 | xxxx | 890 |
| ATOM | 891 | N | GLY | A | 115 | 32.617 | 6.521 | 79.848 | 1.00 | 0.00 | xxxx | 891 |
| ATOM | 892 | CA | GLY | A | 115 | 31.920 | 5.842 | 78.762 | 1.00 | 0.00 | xxxx | 892 |
| ATOM | 893 | C | GLY | A | 115 | 32.049 | 4.333 | 78.862 | 1.00 | 0.00 | xxxx | 893 |
| ATOM | 894 | O | GLY | A | 115 | 31.061 | 3.602 | 78.700 | 1.00 | 0.00 | xxxx | 894 |
| ATOM | 895 | N | ILE | A | 116 | 33.252 | 3.848 | 79.163 | 1.00 | 0.00 | xxxx | 895 |
| ATOM | 896 | CA | ILE | A | 116 | 33.454 | 2.405 | 79.314 | 1.00 | 0.00 | xxxx | 896 |
| ATOM | 897 | C | ILE | A | 116 | 32.591 | 1.865 | 80.443 | 1.00 | 0.00 | xxxx | 897 |
| ATOM | 898 | O | ILE | A | 116 | 31.934 | 0.825 | 80.301 | 1.00 | 0.00 | xxxx | 898 |
| ATOM | 899 | CB | ILE | A | 116 | 34.940 | 2.081 | 79.537 | 1.00 | 0.00 | xxxx | 899 |
| ATOM | 900 | CG1 | ILE | A | 116 | 35.755 | 2.410 | 78.283 | 1.00 | 0.00 | xxxx | 900 |
| ATOM | 901 | CG2 | ILE | A | 116 | 35.112 | 0.620 | 79.957 | 1.00 | 0.00 | xxxx | 901 |
| ATOM | 902 | CD1 | ILE | A | 116 | 37.259 | 2.474 | 78.545 | 1.00 | 0.00 | xxxx | 902 |
| ATOM | 903 | N | LEU | A | 117 | 32.590 | 2.549 | 81.588 | 1.00 | 0.00 | xxxx | 903 |
| ATOM | 904 | CA | LEU | A | 117 | 31.816 | 2.045 | 82.721 | 1.00 | 0.00 | xxxx | 904 |
| ATOM | 905 | C | LEU | A | 117 | 30.326 | 2.034 | 82.407 | 1.00 | 0.00 | xxxx | 905 |
| ATOM | 906 | O | LEU | A | 117 | 29.605 | 1.110 | 82.800 | 1.00 | 0.00 | xxxx | 906 |
| ATOM | 907 | CB | LEU | A | 117 | 32.116 | 2.869 | 83.974 | 1.00 | 0.00 | xxxx | 907 |
| ATOM | 908 | CG | LEU | A | 117 | 33.569 | 2.740 | 84.443 | 1.00 | 0.00 | xxxx | 908 |
| ATOM | 909 | CD1 | LEU | A | 117 | 33.871 | 3.740 | 85.536 | 1.00 | 0.00 | xxxx | 909 |
| ATOM | 910 | CD2 | LEU | A | 117 | 33.858 | 1.322 | 84.933 | 1.00 | 0.00 | xxxx | 910 |
| ATOM | 911 | N | GLN | A | 118 | 29.852 | 3.033 | 81.669 | 1.00 | 0.00 | xxxx | 911 |
| ATOM | 912 | CA | GLN | A | 118 | 28.444 | 3.055 | 81.301 | 1.00 | 0.00 | xxxx | 912 |
| ATOM | 913 | C | GLN | A | 118 | 28.099 | 1.925 | 80.332 | 1.00 | 0.00 | xxxx | 913 |
| ATOM | 914 | O | GLN | A | 118 | 27.021 | 1.323 | 80.431 | 1.00 | 0.00 | xxxx | 914 |
| ATOM | 915 | CB | GLN | A | 118 | 28.100 | 4.415 | 80.706 | 1.00 | 0.00 | xxxx | 915 |
| ATOM | 916 | CG | GLN | A | 118 | 26.672 | 4.525 | 80.317 | 1.00 | 0.00 | xxxx | 916 |
| ATOM | 917 | CD | GLN | A | 118 | 26.359 | 5.813 | 79.604 | 1.00 | 0.00 | xxxx | 917 |
| ATOM | 918 | OE1 | GLN | A | 118 | 26.587 | 5.942 | 78.406 | 1.00 | 0.00 | xxxx | 918 |
| ATOM | 919 | NE2 | GLN | A | 118 | 25.821 | 6.774 | 80.339 | 1.00 | 0.00 | xxxx | 919 |
| ATOM | 920 | N | GLY | A | 119 | 28.997 | 1.622 | 79.391 | 1.00 | 0.00 | xxxx | 920 |
| ATOM | 921 | CA | GLY | A | 119 | 28.759 | 0.516 | 78.490 | 1.00 | 0.00 | xxxx | 921 |
| ATOM | 922 | C | GLY | A | 119 | 28.777 | −0.821 | 79.199 | 1.00 | 0.00 | xxxx | 922 |
| ATOM | 923 | O | GLY | A | 119 | 28.010 | −1.722 | 78.851 | 1.00 | 0.00 | xxxx | 923 |
| ATOM | 924 | N | GLN | A | 120 | 29.643 | −0.969 | 80.210 | 1.00 | 0.00 | xxxx | 924 |
| ATOM | 925 | CA | GLN | A | 120 | 29.658 | −2.210 | 80.978 | 1.00 | 0.00 | xxxx | 925 |
| ATOM | 926 | C | GLN | A | 120 | 28.335 | −2.435 | 81.700 | 1.00 | 0.00 | xxxx | 926 |
| ATOM | 927 | O | GLN | A | 120 | 27.832 | −3.566 | 81.743 | 1.00 | 0.00 | xxxx | 927 |
| ATOM | 928 | CB | GLN | A | 120 | 30.821 | −2.219 | 81.964 | 1.00 | 0.00 | xxxx | 928 |
| ATOM | 929 | CG | GLN | A | 120 | 32.178 | −2.283 | 81.299 | 1.00 | 0.00 | xxxx | 929 |
| ATOM | 930 | CD | GLN | A | 120 | 33.308 | −2.245 | 82.300 | 1.00 | 0.00 | xxxx | 930 |
| ATOM | 931 | OE1 | GLN | A | 120 | 33.092 | −2.012 | 83.493 | 1.00 | 0.00 | xxxx | 931 |
| ATOM | 932 | NE2 | GLN | A | 120 | 34.526 | −2.486 | 81.827 | 1.00 | 0.00 | xxxx | 932 |
| ATOM | 933 | N | ILE | A | 121 | 27.765 | −1.384 | 82.289 | 1.00 | 0.00 | xxxx | 933 |
| ATOM | 934 | CA | ILE | A | 121 | 26.481 | −1.522 | 82.973 | 1.00 | 0.00 | xxxx | 934 |
| ATOM | 935 | C | ILE | A | 121 | 25.433 | −2.060 | 82.012 | 1.00 | 0.00 | xxxx | 935 |
| ATOM | 936 | O | ILE | A | 121 | 24.703 | −3.015 | 82.318 | 1.00 | 0.00 | xxxx | 936 |
| ATOM | 937 | CB | ILE | A | 121 | 26.044 | −0.170 | 83.567 | 1.00 | 0.00 | xxxx | 937 |
| ATOM | 938 | CG1 | ILE | A | 121 | 26.980 | 0.263 | 84.696 | 1.00 | 0.00 | xxxx | 938 |
| ATOM | 939 | CG2 | ILE | A | 121 | 24.601 | −0.259 | 84.071 | 1.00 | 0.00 | xxxx | 939 |
| ATOM | 940 | CD1 | ILE | A | 121 | 26.783 | 1.722 | 85.103 | 1.00 | 0.00 | xxxx | 940 |
| ATOM | 941 | N | MET | A | 122 | 25.338 | −1.450 | 80.831 | 1.00 | 0.00 | xxxx | 941 |
| ATOM | 942 | CA | MET | A | 122 | 24.306 | −1.857 | 79.886 | 1.00 | 0.00 | xxxx | 942 |
| ATOM | 943 | C | MET | A | 122 | 24.573 | −3.252 | 79.344 | 1.00 | 0.00 | xxxx | 943 |
| ATOM | 944 | O | MET | A | 122 | 23.640 | −4.049 | 79.180 | 1.00 | 0.00 | xxxx | 944 |
| ATOM | 945 | CB | MET | A | 122 | 24.209 | −0.852 | 78.742 | 1.00 | 0.00 | xxxx | 945 |
| ATOM | 946 | CG | MET | A | 122 | 23.000 | −1.108 | 77.851 | 1.00 | 0.00 | xxxx | 946 |
| ATOM | 947 | SD | MET | A | 122 | 21.435 | −0.672 | 78.643 | 1.00 | 0.00 | xxxx | 947 |
| ATOM | 948 | CE | MET | A | 122 | 21.453 | 1.110 | 78.453 | 1.00 | 0.00 | xxxx | 948 |
| ATOM | 949 | N | ALA | A | 123 | 25.839 | −3.564 | 79.049 | 1.00 | 0.00 | xxxx | 949 |
| ATOM | 950 | CA | ALA | A | 123 | 26.157 | −4.867 | 78.473 | 1.00 | 0.00 | xxxx | 950 |
| ATOM | 951 | C | ALA | A | 123 | 25.888 | −5.980 | 79.471 | 1.00 | 0.00 | xxxx | 951 |
| ATOM | 952 | O | ALA | A | 123 | 25.370 | −7.043 | 79.104 | 1.00 | 0.00 | xxxx | 952 |
| ATOM | 953 | CB | ALA | A | 123 | 27.618 | −4.898 | 78.014 | 1.00 | 0.00 | xxxx | 953 |
| ATOM | 954 | N | ASP | A | 124 | 26.216 | −5.746 | 80.744 | 1.00 | 0.00 | xxxx | 954 |
| ATOM | 955 | CA | ASP | A | 124 | 25.959 | −6.760 | 81.759 | 1.00 | 0.00 | xxxx | 955 |
| ATOM | 956 | C | ASP | A | 124 | 24.465 | −6.967 | 81.961 | 1.00 | 0.00 | xxxx | 956 |
| ATOM | 957 | O | ASP | A | 124 | 24.005 | −8.103 | 82.152 | 1.00 | 0.00 | xxxx | 957 |
| ATOM | 958 | CB | ASP | A | 124 | 26.654 | −6.386 | 83.067 | 1.00 | 0.00 | xxxx | 958 |
| ATOM | 959 | CG | ASP | A | 124 | 28.160 | −6.575 | 82.996 | 1.00 | 0.00 | xxxx | 959 |
| ATOM | 960 | OD1 | ASP | A | 124 | 28.633 | −7.287 | 82.084 | 1.00 | 0.00 | xxxx | 960 |
| ATOM | 961 | OD2 | ASP | A | 124 | 28.877 | −6.021 | 83.851 | 1.00 | 0.00 | xxxx | 961 |
| ATOM | 962 | N | TYR | A | 125 | 23.681 | −5.890 | 81.898 | 1.00 | 0.00 | xxxx | 962 |
| ATOM | 963 | CA | TYR | A | 125 | 22.236 | −6.059 | 81.995 | 1.00 | 0.00 | xxxx | 963 |
| ATOM | 964 | C | TYR | A | 125 | 21.709 | −6.872 | 80.819 | 1.00 | 0.00 | xxxx | 964 |
| ATOM | 965 | O | TYR | A | 125 | 20.909 | −7.803 | 80.994 | 1.00 | 0.00 | xxxx | 965 |
| ATOM | 966 | CB | TYR | A | 125 | 21.544 | −4.692 | 82.073 | 1.00 | 0.00 | xxxx | 966 |
| ATOM | 967 | CG | TYR | A | 125 | 20.049 | −4.820 | 81.878 | 1.00 | 0.00 | xxxx | 967 |
| ATOM | 968 | CD1 | TYR | A | 125 | 19.209 | −5.126 | 82.944 | 1.00 | 0.00 | xxxx | 968 |
| ATOM | 969 | CD2 | TYR | A | 125 | 19.487 | −4.695 | 80.614 | 1.00 | 0.00 | xxxx | 969 |
| ATOM | 970 | CE1 | TYR | A | 125 | 17.838 | −5.281 | 82.746 | 1.00 | 0.00 | xxxx | 970 |
| ATOM | 971 | CE2 | TYR | A | 125 | 18.132 | −4.858 | 80.411 | 1.00 | 0.00 | xxxx | 971 |
| ATOM | 972 | CZ | TYR | A | 125 | 17.323 | −5.140 | 81.472 | 1.00 | 0.00 | xxxx | 972 |
| ATOM | 973 | OH | TYR | A | 125 | 15.974 | −5.294 | 81.249 | 1.00 | 0.00 | xxxx | 973 |
| ATOM | 974 | N | TRP | A | 126 | 22.144 | −6.522 | 79.607 | 1.00 | 0.00 | xxxx | 974 |
| ATOM | 975 | CA | TRP | A | 126 | 21.697 | −7.212 | 78.402 | 1.00 | 0.00 | xxxx | 975 |
| ATOM | 976 | C | TRP | A | 126 | 21.984 | −8.704 | 78.489 | 1.00 | 0.00 | xxxx | 976 |
| ATOM | 977 | O | TRP | A | 126 | 21.119 | −9.535 | 78.184 | 1.00 | 0.00 | xxxx | 977 |
| ATOM | 978 | CB | TRP | A | 126 | 22.395 | −6.598 | 77.184 | 1.00 | 0.00 | xxxx | 978 |
| ATOM | 979 | CG | TRP | A | 126 | 22.086 | −7.259 | 75.841 | 1.00 | 0.00 | xxxx | 979 |
| ATOM | 980 | CD1 | TRP | A | 126 | 22.802 | −8.247 | 75.234 | 1.00 | 0.00 | xxxx | 980 |
| ATOM | 981 | CD2 | TRP | A | 126 | 21.009 | −6.938 | 74.949 | 1.00 | 0.00 | xxxx | 981 |
| ATOM | 982 | NE1 | TRP | A | 126 | 22.229 | −8.578 | 74.023 | 1.00 | 0.00 | xxxx | 982 |
| ATOM | 983 | CE2 | TRP | A | 126 | 21.131 | −7.782 | 73.827 | 1.00 | 0.00 | xxxx | 983 |
| ATOM | 984 | CE3 | TRP | A | 126 | 19.953 | −6.027 | 74.995 | 1.00 | 0.00 | xxxx | 984 |
| ATOM | 985 | CZ2 | TRP | A | 126 | 20.231 | −7.746 | 72.762 | 1.00 | 0.00 | xxxx | 985 |
| ATOM | 986 | CZ3 | TRP | A | 126 | 19.054 | −5.988 | 73.925 | 1.00 | 0.00 | xxxx | 986 |
| ATOM | 987 | CH2 | TRP | A | 126 | 19.206 | −6.841 | 72.832 | 1.00 | 0.00 | xxxx | 987 |
| ATOM | 988 | N | LYS | A | 127 | 23.197 | −9.063 | 78.918 | 1.00 | 0.00 | xxxx | 988 |
| ATOM | 989 | CA | LYS | A | 127 | 23.576 | −10.473 | 78.961 | 1.00 | 0.00 | xxxx | 989 |
| ATOM | 990 | C | LYS | A | 127 | 22.768 | −11.247 | 79.995 | 1.00 | 0.00 | xxxx | 990 |
| ATOM | 991 | O | LYS | A | 127 | 22.540 | −12.453 | 79.826 | 1.00 | 0.00 | xxxx | 991 |
| ATOM | 992 | CB | LYS | A | 127 | 25.073 | −10.596 | 79.250 | 1.00 | 0.00 | xxxx | 992 |
| ATOM | 993 | CG | LYS | A | 127 | 25.956 | −10.147 | 78.103 | 1.00 | 0.00 | xxxx | 993 |
| ATOM | 994 | CD | LYS | A | 127 | 27.414 | −10.102 | 78.521 | 1.00 | 0.00 | xxxx | 994 |
| ATOM | 995 | CE | LYS | A | 127 | 28.307 | −9.804 | 77.331 | 1.00 | 0.00 | xxxx | 995 |
| ATOM | 996 | NZ | LYS | A | 127 | 29.742 | −9.750 | 77.743 | 1.00 | 0.00 | xxxx | 996 |
| ATOM | 997 | N | ALA | A | 128 | 22.332 | −10.583 | 81.067 | 1.00 | 0.00 | xxxx | 997 |
| ATOM | 998 | CA | ALA | A | 128 | 21.615 | −11.253 | 82.147 | 1.00 | 0.00 | xxxx | 998 |
| ATOM | 999 | C | ALA | A | 128 | 20.110 | −11.293 | 81.937 | 1.00 | 0.00 | xxxx | 999 |
| ATOM | 1000 | O | ALA | A | 128 | 19.420 | −12.022 | 82.657 | 1.00 | 0.00 | xxxx | 1000 |
| ATOM | 1001 | CB | ALA | A | 128 | 21.898 | −10.540 | 83.473 | 1.00 | 0.00 | xxxx | 1001 |
| ATOM | 1002 | N | HIS | A | 129 | 19.583 | −10.530 | 80.984 | 1.00 | 0.00 | xxxx | 1002 |
| ATOM | 1003 | CA | HIS | A | 129 | 18.139 | −10.395 | 80.799 | 1.00 | 0.00 | xxxx | 1003 |
| ATOM | 1004 | C | HIS | A | 129 | 17.763 | −10.624 | 79.343 | 1.00 | 0.00 | xxxx | 1004 |
| ATOM | 1005 | O | HIS | A | 129 | 17.662 | −9.671 | 78.558 | 1.00 | 0.00 | xxxx | 1005 |
| ATOM | 1006 | CB | HIS | A | 129 | 17.662 | −9.033 | 81.295 | 1.00 | 0.00 | xxxx | 1006 |
| ATOM | 1007 | CG | HIS | A | 129 | 17.943 | −8.800 | 82.743 | 1.00 | 0.00 | xxxx | 1007 |
| ATOM | 1008 | ND1 | HIS | A | 129 | 17.015 | −9.057 | 83.730 | 1.00 | 0.00 | xxxx | 1008 |
| ATOM | 1009 | CD2 | HIS | A | 129 | 19.060 | −8.370 | 83.377 | 1.00 | 0.00 | xxxx | 1009 |
| ATOM | 1010 | CE1 | HIS | A | 129 | 17.544 | −8.781 | 84.909 | 1.00 | 0.00 | xxxx | 1010 |
| ATOM | 1011 | NE2 | HIS | A | 129 | 18.782 | −8.363 | 84.723 | 1.00 | 0.00 | xxxx | 1011 |
| ATOM | 1012 | N | PRO | A | 130 | 17.512 | −11.875 | 78.955 | 1.00 | 0.00 | xxxx | 1012 |
| ATOM | 1013 | CA | PRO | A | 130 | 17.138 | −12.147 | 77.556 | 1.00 | 0.00 | xxxx | 1013 |
| ATOM | 1014 | C | PRO | A | 130 | 15.891 | −11.409 | 77.117 | 1.00 | 0.00 | xxxx | 1014 |
| ATOM | 1015 | O | PRO | A | 130 | 15.751 | −11.087 | 75.930 | 1.00 | 0.00 | xxxx | 1015 |
| ATOM | 1016 | CB | PRO | A | 130 | 16.922 | −13.667 | 77.532 | 1.00 | 0.00 | xxxx | 1016 |
| ATOM | 1017 | CG | PRO | A | 130 | 17.575 | −14.187 | 78.772 | 1.00 | 0.00 | xxxx | 1017 |
| ATOM | 1018 | CD | PRO | A | 130 | 17.522 | −13.090 | 79.785 | 1.00 | 0.00 | xxxx | 1018 |
| ATOM | 1019 | N | GLU | A | 131 | 14.981 | −11.120 | 78.046 | 1.00 | 0.00 | xxxx | 1019 |
| ATOM | 1020 | CA | GLU | A | 131 | 13.760 | −10.409 | 77.702 | 1.00 | 0.00 | xxxx | 1020 |
| ATOM | 1021 | C | GLU | A | 131 | 14.022 | −8.977 | 77.257 | 1.00 | 0.00 | xxxx | 1021 |
| ATOM | 1022 | O | GLU | A | 131 | 13.108 | −8.337 | 76.722 | 1.00 | 0.00 | xxxx | 1022 |
| ATOM | 1023 | CB | GLU | A | 131 | 12.791 | −10.429 | 78.885 | 1.00 | 0.00 | xxxx | 1023 |
| ATOM | 1024 | CG | GLU | A | 131 | 13.175 | −9.489 | 80.025 | 1.00 | 0.00 | xxxx | 1024 |
| ATOM | 1025 | CD | GLU | A | 131 | 14.111 | −10.120 | 81.050 | 1.00 | 0.00 | xxxx | 1025 |
| ATOM | 1026 | OE1 | GLU | A | 131 | 14.789 | −11.127 | 80.739 | 1.00 | 0.00 | xxxx | 1026 |
| ATOM | 1027 | OE2 | GLU | A | 131 | 14.164 | −9.598 | 82.186 | 1.00 | 0.00 | xxxx | 1027 |
| ATOM | 1028 | N | ALA | A | 132 | 15.240 | −8.470 | 77.452 | 1.00 | 0.00 | xxxx | 1028 |
| ATOM | 1029 | CA | ALA | A | 132 | 15.558 | −7.112 | 77.019 | 1.00 | 0.00 | xxxx | 1029 |
| ATOM | 1030 | C | ALA | A | 132 | 15.533 | −6.981 | 75.502 | 1.00 | 0.00 | xxxx | 1030 |
| ATOM | 1031 | O | ALA | A | 132 | 15.259 | −5.893 | 74.977 | 1.00 | 0.00 | xxxx | 1031 |
| ATOM | 1032 | CB | ALA | A | 132 | 16.920 | −6.693 | 77.571 | 1.00 | 0.00 | xxxx | 1032 |
| ATOM | 1033 | N | ASP | A | 133 | 15.820 | −8.070 | 74.788 | 1.00 | 0.00 | xxxx | 1033 |
| ATOM | 1034 | CA | ASP | A | 133 | 15.782 | −8.100 | 73.326 | 1.00 | 0.00 | xxxx | 1034 |
| ATOM | 1035 | C | ASP | A | 133 | 14.326 | −8.311 | 72.915 | 1.00 | 0.00 | xxxx | 1035 |
| ATOM | 1036 | O | ASP | A | 133 | 13.884 | −9.410 | 72.571 | 1.00 | 0.00 | xxxx | 1036 |
| ATOM | 1037 | CB | ASP | A | 133 | 16.710 | −9.194 | 72.816 | 1.00 | 0.00 | xxxx | 1037 |
| ATOM | 1038 | CG | ASP | A | 133 | 16.793 | −9.243 | 71.311 | 1.00 | 0.00 | xxxx | 1038 |
| ATOM | 1039 | OD1 | ASP | A | 133 | 16.448 | −8.238 | 70.665 | 1.00 | 0.00 | xxxx | 1039 |
| ATOM | 1040 | OD2 | ASP | A | 133 | 17.207 | −10.292 | 70.769 | 1.00 | 0.00 | xxxx | 1040 |
| ATOM | 1041 | N | LYS | A | 134 | 13.564 | −7.217 | 72.960 | 1.00 | 0.00 | xxxx | 1041 |
| ATOM | 1042 | CA | LYS | A | 134 | 12.112 | −7.327 | 72.850 | 1.00 | 0.00 | xxxx | 1042 |
| ATOM | 1043 | C | LYS | A | 134 | 11.679 | −7.919 | 71.514 | 1.00 | 0.00 | xxxx | 1043 |
| ATOM | 1044 | O | LYS | A | 134 | 10.719 | −8.697 | 71.460 | 1.00 | 0.00 | xxxx | 1044 |
| ATOM | 1045 | CB | LYS | A | 134 | 11.446 | −5.975 | 73.106 | 1.00 | 0.00 | xxxx | 1045 |
| ATOM | 1046 | CG | LYS | A | 134 | 11.290 | −5.645 | 74.582 | 1.00 | 0.00 | xxxx | 1046 |
| ATOM | 1047 | CD | LYS | A | 134 | 10.626 | −4.292 | 74.777 | 1.00 | 0.00 | xxxx | 1047 |
| ATOM | 1048 | CE | LYS | A | 134 | 11.499 | −3.172 | 74.246 | 1.00 | 0.00 | xxxx | 1048 |
| ATOM | 1049 | NZ | LYS | A | 134 | 10.924 | −1.840 | 74.594 | 1.00 | 0.00 | xxxx | 1049 |
| ATOM | 1050 | N | ASN | A | 135 | 12.370 | −7.572 | 70.428 | 1.00 | 0.00 | xxxx | 1050 |
| ATOM | 1051 | CA | ASN | A | 135 | 11.992 | −8.084 | 69.112 | 1.00 | 0.00 | xxxx | 1051 |
| ATOM | 1052 | C | ASN | A | 135 | 12.741 | −9.357 | 68.735 | 1.00 | 0.00 | xxxx | 1052 |
| ATOM | 1053 | O | ASN | A | 135 | 12.604 | −9.833 | 67.603 | 1.00 | 0.00 | xxxx | 1053 |
| ATOM | 1054 | CB | ASN | A | 135 | 12.122 | −7.014 | 68.020 | 1.00 | 0.00 | xxxx | 1054 |
| ATOM | 1055 | CG | ASN | A | 135 | 13.561 | −6.704 | 67.666 | 1.00 | 0.00 | xxxx | 1055 |
| ATOM | 1056 | OD1 | ASN | A | 135 | 14.467 | −6.947 | 68.452 | 1.00 | 0.00 | xxxx | 1056 |
| ATOM | 1057 | ND2 | ASN | A | 135 | 13.774 | −6.149 | 66.479 | 1.00 | 0.00 | xxxx | 1057 |
| ATOM | 1058 | N | HIS | A | 136 | 13.522 | −9.917 | 69.662 | 1.00 | 0.00 | xxxx | 1058 |
| ATOM | 1059 | CA | HIS | A | 136 | 14.134 | −11.237 | 69.512 | 1.00 | 0.00 | xxxx | 1059 |
| ATOM | 1060 | C | HIS | A | 136 | 15.038 | −11.338 | 68.285 | 1.00 | 0.00 | xxxx | 1060 |
| ATOM | 1061 | O | HIS | A | 136 | 15.151 | −12.405 | 67.677 | 1.00 | 0.00 | xxxx | 1061 |
| ATOM | 1062 | CB | HIS | A | 136 | 13.080 | −12.350 | 69.509 | 1.00 | 0.00 | xxxx | 1062 |
| ATOM | 1063 | CG | HIS | A | 136 | 12.084 | −12.240 | 70.621 | 1.00 | 0.00 | xxxx | 1063 |
| ATOM | 1064 | ND1 | HIS | A | 136 | 12.436 | −12.352 | 71.949 | 1.00 | 0.00 | xxxx | 1064 |
| ATOM | 1065 | CD2 | HIS | A | 136 | 10.746 | −12.032 | 70.602 | 1.00 | 0.00 | xxxx | 1065 |
| ATOM | 1066 | CE1 | HIS | A | 136 | 11.358 | −12.214 | 72.701 | 1.00 | 0.00 | xxxx | 1066 |
| ATOM | 1067 | NE2 | HIS | A | 136 | 10.319 | −12.020 | 71.908 | 1.00 | 0.00 | xxxx | 1067 |
| ATOM | 1068 | N | ASP | A | 137 | 15.700 | −10.243 | 67.908 | 1.00 | 0.00 | xxxx | 1068 |
| ATOM | 1069 | CA | ASP | A | 137 | 16.562 | −10.248 | 66.733 | 1.00 | 0.00 | xxxx | 1069 |
| ATOM | 1070 | C | ASP | A | 137 | 18.042 | −10.399 | 67.064 | 1.00 | 0.00 | xxxx | 1070 |
| ATOM | 1071 | O | ASP | A | 137 | 18.867 | −10.406 | 66.145 | 1.00 | 0.00 | xxxx | 1071 |
| ATOM | 1072 | CB | ASP | A | 137 | 16.307 | −9.015 | 65.842 | 1.00 | 0.00 | xxxx | 1072 |
| ATOM | 1073 | CG | ASP | A | 137 | 16.759 | −7.702 | 66.476 | 1.00 | 0.00 | xxxx | 1073 |
| ATOM | 1074 | OD1 | ASP | A | 137 | 17.124 | −7.680 | 67.671 | 1.00 | 0.00 | xxxx | 1074 |
| ATOM | 1075 | OD2 | ASP | A | 137 | 16.738 | −6.676 | 65.760 | 1.00 | 0.00 | xxxx | 1075 |
| ATOM | 1076 | N | GLY | A | 138 | 18.398 | −10.520 | 68.345 | 1.00 | 0.00 | xxxx | 1076 |
| ATOM | 1077 | CA | GLY | A | 138 | 19.792 | −10.644 | 68.720 | 1.00 | 0.00 | xxxx | 1077 |
| ATOM | 1078 | C | GLY | A | 138 | 20.591 | −9.366 | 68.624 | 1.00 | 0.00 | xxxx | 1078 |
| ATOM | 1079 | O | GLY | A | 138 | 21.826 | −9.417 | 68.631 | 1.00 | 0.00 | xxxx | 1079 |
| ATOM | 1080 | N | VAL | A | 139 | 19.923 | −8.221 | 68.520 | 1.00 | 0.00 | xxxx | 1080 |
| ATOM | 1081 | CA | VAL | A | 139 | 20.562 | −6.922 | 68.351 | 1.00 | 0.00 | xxxx | 1081 |
| ATOM | 1082 | C | VAL | A | 139 | 19.945 | −5.964 | 69.360 | 1.00 | 0.00 | xxxx | 1082 |
| ATOM | 1083 | O | VAL | A | 139 | 18.726 | −5.970 | 69.560 | 1.00 | 0.00 | xxxx | 1083 |
| ATOM | 1084 | CB | VAL | A | 139 | 20.342 | −6.386 | 66.917 | 1.00 | 0.00 | xxxx | 1084 |
| ATOM | 1085 | CG1 | VAL | A | 139 | 20.987 | −5.023 | 66.742 | 1.00 | 0.00 | xxxx | 1085 |
| ATOM | 1086 | CG2 | VAL | A | 139 | 20.877 | −7.353 | 65.882 | 1.00 | 0.00 | xxxx | 1086 |
| ATOM | 1087 | N | MET | A | 140 | 20.775 | −5.124 | 69.979 | 1.00 | 0.00 | xxxx | 1087 |
| ATOM | 1088 | CA | MET | A | 140 | 20.291 | −4.122 | 70.927 | 1.00 | 0.00 | xxxx | 1088 |
| ATOM | 1089 | C | MET | A | 140 | 19.945 | −2.835 | 70.175 | 1.00 | 0.00 | xxxx | 1089 |
| ATOM | 1090 | O | MET | A | 140 | 20.842 | −2.119 | 69.723 | 1.00 | 0.00 | xxxx | 1090 |
| ATOM | 1091 | CB | MET | A | 140 | 21.358 | −3.856 | 71.989 | 1.00 | 0.00 | xxxx | 1091 |
| ATOM | 1092 | CG | MET | A | 140 | 21.000 | −2.732 | 72.964 | 1.00 | 0.00 | xxxx | 1092 |
| ATOM | 1093 | SD | MET | A | 140 | 22.400 | −2.094 | 73.929 | 1.00 | 0.00 | xxxx | 1093 |
| ATOM | 1094 | CE | MET | A | 140 | 22.726 | −3.488 | 74.998 | 1.00 | 0.00 | xxxx | 1094 |
| ATOM | 1095 | N | GLN | A | 141 | 18.650 | −2.525 | 70.064 | 1.00 | 0.00 | xxxx | 1095 |
| ATOM | 1096 | CA | GLN | A | 141 | 18.204 | −1.251 | 69.491 | 1.00 | 0.00 | xxxx | 1096 |
| ATOM | 1097 | C | GLN | A | 141 | 18.274 | −0.171 | 70.563 | 1.00 | 0.00 | xxxx | 1097 |
| ATOM | 1098 | O | GLN | A | 141 | 17.623 | −0.295 | 71.606 | 1.00 | 0.00 | xxxx | 1098 |
| ATOM | 1099 | CB | GLN | A | 141 | 16.764 | −1.368 | 68.989 | 1.00 | 0.00 | xxxx | 1099 |
| ATOM | 1100 | CG | GLN | A | 141 | 16.604 | −1.910 | 67.573 | 1.00 | 0.00 | xxxx | 1100 |
| ATOM | 1101 | CD | GLN | A | 141 | 16.779 | −3.409 | 67.477 | 1.00 | 0.00 | xxxx | 1101 |
| ATOM | 1102 | OE1 | GLN | A | 141 | 16.319 | −4.156 | 68.337 | 1.00 | 0.00 | xxxx | 1102 |
| ATOM | 1103 | NE2 | GLN | A | 141 | 17.405 | −3.866 | 66.395 | 1.00 | 0.00 | xxxx | 1103 |
| ATOM | 1104 | N | TYR | A | 142 | 19.048 | 0.886 | 70.324 | 1.00 | 0.00 | xxxx | 1104 |
| ATOM | 1105 | CA | TYR | A | 142 | 19.253 | 1.886 | 71.361 | 1.00 | 0.00 | xxxx | 1105 |
| ATOM | 1106 | C | TYR | A | 142 | 19.068 | 3.293 | 70.811 | 1.00 | 0.00 | xxxx | 1106 |
| ATOM | 1107 | O | TYR | A | 142 | 19.085 | 3.532 | 69.599 | 1.00 | 0.00 | xxxx | 1107 |
| ATOM | 1108 | CB | TYR | A | 142 | 20.644 | 1.775 | 71.996 | 1.00 | 0.00 | xxxx | 1108 |
| ATOM | 1109 | CG | TYR | A | 142 | 21.784 | 2.201 | 71.095 | 1.00 | 0.00 | xxxx | 1109 |
| ATOM | 1110 | CD1 | TYR | A | 142 | 22.257 | 3.508 | 71.115 | 1.00 | 0.00 | xxxx | 1110 |
| ATOM | 1111 | CD2 | TYR | A | 142 | 22.402 | 1.298 | 70.239 | 1.00 | 0.00 | xxxx | 1111 |
| ATOM | 1112 | CE1 | TYR | A | 142 | 23.303 | 3.906 | 70.304 | 1.00 | 0.00 | xxxx | 1112 |
| ATOM | 1113 | CE2 | TYR | A | 142 | 23.450 | 1.681 | 69.428 | 1.00 | 0.00 | xxxx | 1113 |
| ATOM | 1114 | CZ | TYR | A | 142 | 23.894 | 2.990 | 69.456 | 1.00 | 0.00 | xxxx | 1114 |
| ATOM | 1115 | OH | TYR | A | 142 | 24.936 | 3.386 | 68.661 | 1.00 | 0.00 | xxxx | 1115 |
| ATOM | 1116 | N | VAL | A | 143 | 18.883 | 4.230 | 71.738 | 1.00 | 0.00 | xxxx | 1116 |
| ATOM | 1117 | CA | VAL | A | 143 | 18.962 | 5.652 | 71.435 | 1.00 | 0.00 | xxxx | 1117 |
| ATOM | 1118 | C | VAL | A | 143 | 20.031 | 6.273 | 72.337 | 1.00 | 0.00 | xxxx | 1118 |
| ATOM | 1119 | O | VAL | A | 143 | 20.359 | 5.757 | 73.414 | 1.00 | 0.00 | xxxx | 1119 |
| ATOM | 1120 | CB | VAL | A | 143 | 17.605 | 6.379 | 71.563 | 1.00 | 0.00 | xxxx | 1120 |
| ATOM | 1121 | CG1 | VAL | A | 143 | 16.575 | 5.795 | 70.582 | 1.00 | 0.00 | xxxx | 1121 |
| ATOM | 1122 | CG2 | VAL | A | 143 | 17.088 | 6.309 | 73.005 | 1.00 | 0.00 | xxxx | 1122 |
| ATOM | 1123 | N | MET | A | 144 | 20.561 | 7.412 | 71.891 | 1.00 | 0.00 | xxxx | 1123 |
| ATOM | 1124 | CA | MET | A | 144 | 21.693 | 8.058 | 72.540 | 1.00 | 0.00 | xxxx | 1124 |
| ATOM | 1125 | C | MET | A | 144 | 21.416 | 9.548 | 72.683 | 1.00 | 0.00 | xxxx | 1125 |
| ATOM | 1126 | O | MET | A | 144 | 21.234 | 10.247 | 71.675 | 1.00 | 0.00 | xxxx | 1126 |
| ATOM | 1127 | CB | MET | A | 144 | 22.955 | 7.829 | 71.701 | 1.00 | 0.00 | xxxx | 1127 |
| ATOM | 1128 | CG | MET | A | 144 | 24.158 | 8.670 | 72.136 | 1.00 | 0.00 | xxxx | 1128 |
| ATOM | 1129 | SD | MET | A | 144 | 24.866 | 8.211 | 73.720 | 1.00 | 0.00 | xxxx | 1129 |
| ATOM | 1130 | CE | MET | A | 144 | 25.631 | 6.652 | 73.300 | 1.00 | 0.00 | xxxx | 1130 |
| ATOM | 1131 | N | LEU | A | 145 | 21.407 | 10.030 | 73.924 | 1.00 | 0.00 | xxxx | 1131 |
| ATOM | 1132 | CA | LEU | A | 145 | 21.252 | 11.456 | 74.226 | 1.00 | 0.00 | xxxx | 1132 |
| ATOM | 1133 | C | LEU | A | 145 | 22.628 | 12.034 | 74.512 | 1.00 | 0.00 | xxxx | 1133 |
| ATOM | 1134 | O | LEU | A | 145 | 23.225 | 11.765 | 75.565 | 1.00 | 0.00 | xxxx | 1134 |
| ATOM | 1135 | CB | LEU | A | 145 | 20.324 | 11.660 | 75.424 | 1.00 | 0.00 | xxxx | 1135 |
| ATOM | 1136 | CG | LEU | A | 145 | 18.866 | 11.314 | 75.142 | 1.00 | 0.00 | xxxx | 1136 |
| ATOM | 1137 | CD1 | LEU | A | 145 | 18.133 | 10.928 | 76.428 | 1.00 | 0.00 | xxxx | 1137 |
| ATOM | 1138 | CD2 | LEU | A | 145 | 18.168 | 12.496 | 74.475 | 1.00 | 0.00 | xxxx | 1138 |
| ATOM | 1139 | N | MET | A | 146 | 23.136 | 12.811 | 73.570 | 1.00 | 0.00 | xxxx | 1139 |
| ATOM | 1140 | CA | MET | A | 146 | 24.437 | 13.437 | 73.707 | 1.00 | 0.00 | xxxx | 1140 |
| ATOM | 1141 | C | MET | A | 146 | 24.308 | 14.773 | 74.425 | 1.00 | 0.00 | xxxx | 1141 |
| ATOM | 1142 | O | MET | A | 146 | 23.244 | 15.412 | 74.435 | 1.00 | 0.00 | xxxx | 1142 |
| ATOM | 1143 | CB | MET | A | 146 | 25.059 | 13.655 | 72.327 | 1.00 | 0.00 | xxxx | 1143 |
| ATOM | 1144 | CG | MET | A | 146 | 25.250 | 12.362 | 71.514 | 1.00 | 0.00 | xxxx | 1144 |
| ATOM | 1145 | SD | MET | A | 146 | 26.103 | 12.630 | 69.952 | 1.00 | 0.00 | xxxx | 1145 |
| ATOM | 1146 | CE | MET | A | 146 | 24.844 | 13.565 | 69.077 | 1.00 | 0.00 | xxxx | 1146 |
| ATOM | 1147 | O | GLY | A | 147 | 24.826 | 17.862 | 73.869 | 1.00 | 0.00 | xxxx | 1147 |
| ATOM | 1148 | N | GLY | A | 147 | 25.415 | 15.202 | 75.022 | 1.00 | 0.00 | xxxx | 1148 |
| ATOM | 1149 | CA | GLY | A | 147 | 25.436 | 16.476 | 75.723 | 1.00 | 0.00 | xxxx | 1149 |
| ATOM | 1150 | C | GLY | A | 147 | 25.590 | 17.683 | 74.822 | 1.00 | 0.00 | xxxx | 1150 |
| ATOM | 1151 | N | GLU | A | 148 | 26.569 | 18.521 | 75.124 | 1.00 | 0.00 | xxxx | 1151 |
| ATOM | 1152 | CA | GLU | A | 148 | 26.830 | 19.738 | 74.364 | 1.00 | 0.00 | xxxx | 1152 |
| ATOM | 1153 | C | GLU | A | 148 | 27.800 | 19.435 | 73.230 | 1.00 | 0.00 | xxxx | 1153 |
| ATOM | 1154 | O | GLU | A | 148 | 28.843 | 18.813 | 73.472 | 1.00 | 0.00 | xxxx | 1154 |
| ATOM | 1155 | CB | GLU | A | 148 | 27.474 | 20.786 | 75.259 | 1.00 | 0.00 | xxxx | 1155 |
| ATOM | 1156 | CG | GLU | A | 148 | 26.577 | 21.408 | 76.319 | 1.00 | 0.00 | xxxx | 1156 |
| ATOM | 1157 | CD | GLU | A | 148 | 27.303 | 22.486 | 77.115 | 1.00 | 0.00 | xxxx | 1157 |
| ATOM | 1158 | OE1 | GLU | A | 148 | 28.551 | 22.430 | 77.195 | 1.00 | 0.00 | xxxx | 1158 |
| ATOM | 1159 | OE2 | GLU | A | 148 | 26.631 | 23.394 | 77.651 | 1.00 | 0.00 | xxxx | 1159 |
| ATOM | 1160 | N | PRO | A | 149 | 27.507 | 19.872 | 72.005 | 1.00 | 0.00 | xxxx | 1160 |
| ATOM | 1161 | CA | PRO | A | 149 | 28.435 | 19.633 | 70.884 | 1.00 | 0.00 | xxxx | 1161 |
| ATOM | 1162 | C | PRO | A | 149 | 29.837 | 20.134 | 71.206 | 1.00 | 0.00 | xxxx | 1162 |
| ATOM | 1163 | O | PRO | A | 149 | 30.029 | 21.262 | 71.667 | 1.00 | 0.00 | xxxx | 1163 |
| ATOM | 1164 | CB | PRO | A | 149 | 27.817 | 20.432 | 69.728 | 1.00 | 0.00 | xxxx | 1164 |
| ATOM | 1165 | CG | PRO | A | 149 | 26.358 | 20.518 | 70.061 | 1.00 | 0.00 | xxxx | 1165 |
| ATOM | 1166 | CD | PRO | A | 149 | 26.263 | 20.535 | 71.571 | 1.00 | 0.00 | xxxx | 1166 |
| ATOM | 1167 | N | GLY | A | 150 | 30.819 | 19.267 | 71.005 | 1.00 | 0.00 | xxxx | 1167 |
| ATOM | 1168 | CA | GLY | A | 150 | 32.191 | 19.679 | 71.219 | 1.00 | 0.00 | xxxx | 1168 |
| ATOM | 1169 | C | GLY | A | 150 | 32.672 | 19.657 | 72.657 | 1.00 | 0.00 | xxxx | 1169 |
| ATOM | 1170 | O | GLY | A | 150 | 33.853 | 19.917 | 72.892 | 1.00 | 0.00 | xxxx | 1170 |
| ATOM | 1171 | N | HIS | A | 151 | 31.811 | 19.374 | 73.627 | 1.00 | 0.00 | xxxx | 1171 |
| ATOM | 1172 | CA | HIS | A | 151 | 32.271 | 19.161 | 74.993 | 1.00 | 0.00 | xxxx | 1172 |
| ATOM | 1173 | C | HIS | A | 151 | 33.023 | 17.836 | 75.007 | 1.00 | 0.00 | xxxx | 1173 |
| ATOM | 1174 | O | HIS | A | 151 | 32.548 | 16.854 | 74.436 | 1.00 | 0.00 | xxxx | 1174 |
| ATOM | 1175 | CB | HIS | A | 151 | 31.026 | 19.066 | 75.879 | 1.00 | 0.00 | xxxx | 1175 |
| ATOM | 1176 | CG | HIS | A | 151 | 31.279 | 19.122 | 77.355 | 1.00 | 0.00 | xxxx | 1176 |
| ATOM | 1177 | ND1 | HIS | A | 151 | 32.075 | 18.215 | 78.024 | 1.00 | 0.00 | xxxx | 1177 |
| ATOM | 1178 | CD2 | HIS | A | 151 | 30.743 | 19.927 | 78.305 | 1.00 | 0.00 | xxxx | 1178 |
| ATOM | 1179 | CE1 | HIS | A | 151 | 32.060 | 18.497 | 79.320 | 1.00 | 0.00 | xxxx | 1179 |
| ATOM | 1180 | NE2 | HIS | A | 151 | 31.259 | 19.530 | 79.516 | 1.00 | 0.00 | xxxx | 1180 |
| ATOM | 1181 | N | GLN | A | 152 | 34.205 | 17.798 | 75.636 | 1.00 | 0.00 | xxxx | 1181 |
| ATOM | 1182 | CA | GLN | A | 152 | 34.984 | 16.565 | 75.564 | 1.00 | 0.00 | xxxx | 1182 |
| ATOM | 1183 | C | GLN | A | 152 | 34.239 | 15.390 | 76.189 | 1.00 | 0.00 | xxxx | 1183 |
| ATOM | 1184 | O | GLN | A | 152 | 34.395 | 14.242 | 75.743 | 1.00 | 0.00 | xxxx | 1184 |
| ATOM | 1185 | CB | GLN | A | 152 | 36.365 | 16.753 | 76.199 | 1.00 | 0.00 | xxxx | 1185 |
| ATOM | 1186 | CG | GLN | A | 152 | 36.399 | 16.645 | 77.736 | 1.00 | 0.00 | xxxx | 1186 |
| ATOM | 1187 | CD | GLN | A | 152 | 35.868 | 17.885 | 78.446 | 1.00 | 0.00 | xxxx | 1187 |
| ATOM | 1188 | OE1 | GLN | A | 152 | 35.823 | 18.983 | 77.878 | 1.00 | 0.00 | xxxx | 1188 |
| ATOM | 1189 | NE2 | GLN | A | 152 | 35.489 | 17.713 | 79.712 | 1.00 | 0.00 | xxxx | 1189 |
| ATOM | 1190 | N | ASP | A | 153 | 33.406 | 15.643 | 77.203 | 1.00 | 0.00 | xxxx | 1190 |
| ATOM | 1191 | CA | ASP | A | 153 | 32.689 | 14.532 | 77.822 | 1.00 | 0.00 | xxxx | 1191 |
| ATOM | 1192 | C | ASP | A | 153 | 31.615 | 13.986 | 76.890 | 1.00 | 0.00 | xxxx | 1192 |
| ATOM | 1193 | O | ASP | A | 153 | 31.383 | 12.771 | 76.853 | 1.00 | 0.00 | xxxx | 1193 |
| ATOM | 1194 | CB | ASP | A | 153 | 32.064 | 14.961 | 79.148 | 1.00 | 0.00 | xxxx | 1194 |
| ATOM | 1195 | CG | ASP | A | 153 | 33.095 | 15.277 | 80.215 | 1.00 | 0.00 | xxxx | 1195 |
| ATOM | 1196 | OD1 | ASP | A | 153 | 34.296 | 14.931 | 80.061 | 1.00 | 0.00 | xxxx | 1196 |
| ATOM | 1197 | OD2 | ASP | A | 153 | 32.701 | 15.876 | 81.232 | 1.00 | 0.00 | xxxx | 1197 |
| ATOM | 1198 | N | ALA | A | 154 | 30.964 | 14.862 | 76.115 | 1.00 | 0.00 | xxxx | 1198 |
| ATOM | 1199 | CA | ALA | A | 154 | 29.979 | 14.370 | 75.161 | 1.00 | 0.00 | xxxx | 1199 |
| ATOM | 1200 | C | ALA | A | 154 | 30.644 | 13.545 | 74.074 | 1.00 | 0.00 | xxxx | 1200 |
| ATOM | 1201 | O | ALA | A | 154 | 30.166 | 12.455 | 73.733 | 1.00 | 0.00 | xxxx | 1201 |
| ATOM | 1202 | CB | ALA | A | 154 | 29.177 | 15.525 | 74.558 | 1.00 | 0.00 | xxxx | 1202 |
| ATOM | 1203 | N | ILE | A | 155 | 31.762 | 14.032 | 73.542 | 1.00 | 0.00 | xxxx | 1203 |
| ATOM | 1204 | CA | ILE | A | 155 | 32.466 | 13.305 | 72.491 | 1.00 | 0.00 | xxxx | 1204 |
| ATOM | 1205 | C | ILE | A | 155 | 32.849 | 11.911 | 72.974 | 1.00 | 0.00 | xxxx | 1205 |
| ATOM | 1206 | O | ILE | A | 155 | 32.526 | 10.898 | 72.339 | 1.00 | 0.00 | xxxx | 1206 |
| ATOM | 1207 | CB | ILE | A | 155 | 33.694 | 14.109 | 72.028 | 1.00 | 0.00 | xxxx | 1207 |
| ATOM | 1208 | CG1 | ILE | A | 155 | 33.273 | 15.433 | 71.375 | 1.00 | 0.00 | xxxx | 1208 |
| ATOM | 1209 | CG2 | ILE | A | 155 | 34.527 | 13.289 | 71.054 | 1.00 | 0.00 | xxxx | 1209 |
| ATOM | 1210 | CD1 | ILE | A | 155 | 34.421 | 16.397 | 71.165 | 1.00 | 0.00 | xxxx | 1210 |
| ATOM | 1211 | N | LEU | A | 156 | 33.498 | 11.835 | 74.134 | 1.00 | 0.00 | xxxx | 1211 |
| ATOM | 1212 | CA | LEU | A | 156 | 34.123 | 10.585 | 74.552 | 1.00 | 0.00 | xxxx | 1212 |
| ATOM | 1213 | C | LEU | A | 156 | 33.131 | 9.618 | 75.197 | 1.00 | 0.00 | xxxx | 1213 |
| ATOM | 1214 | O | LEU | A | 156 | 33.229 | 8.403 | 74.991 | 1.00 | 0.00 | xxxx | 1214 |
| ATOM | 1215 | CB | LEU | A | 156 | 35.308 | 10.892 | 75.465 | 1.00 | 0.00 | xxxx | 1215 |
| ATOM | 1216 | CG | LEU | A | 156 | 36.440 | 11.611 | 74.728 | 1.00 | 0.00 | xxxx | 1216 |
| ATOM | 1217 | CD1 | LEU | A | 156 | 37.439 | 12.150 | 75.743 | 1.00 | 0.00 | xxxx | 1217 |
| ATOM | 1218 | CD2 | LEU | A | 156 | 37.134 | 10.715 | 73.700 | 1.00 | 0.00 | xxxx | 1218 |
| ATOM | 1219 | N | ARG | A | 157 | 32.159 | 10.119 | 75.961 | 1.00 | 0.00 | xxxx | 1219 |
| ATOM | 1220 | CA | ARG | A | 157 | 31.169 | 9.206 | 76.539 | 1.00 | 0.00 | xxxx | 1220 |
| ATOM | 1221 | C | ARG | A | 157 | 30.329 | 8.554 | 75.453 | 1.00 | 0.00 | xxxx | 1221 |
| ATOM | 1222 | O | ARG | A | 157 | 29.998 | 7.367 | 75.547 | 1.00 | 0.00 | xxxx | 1222 |
| ATOM | 1223 | CB | ARG | A | 157 | 30.271 | 9.934 | 77.538 | 1.00 | 0.00 | xxxx | 1223 |
| ATOM | 1224 | CG | ARG | A | 157 | 30.988 | 10.314 | 78.814 | 1.00 | 0.00 | xxxx | 1224 |
| ATOM | 1225 | CD | ARG | A | 157 | 30.118 | 11.204 | 79.671 | 1.00 | 0.00 | xxxx | 1225 |
| ATOM | 1226 | NE | ARG | A | 157 | 30.846 | 11.651 | 80.851 | 1.00 | 0.00 | xxxx | 1226 |
| ATOM | 1227 | CZ | ARG | A | 157 | 30.435 | 12.638 | 81.635 | 1.00 | 0.00 | xxxx | 1227 |
| ATOM | 1228 | NH1 | ARG | A | 157 | 29.287 | 13.248 | 81.365 | 1.00 | 0.00 | xxxx | 1228 |
| ATOM | 1229 | NH2 | ARG | A | 157 | 31.153 | 13.017 | 82.682 | 1.00 | 0.00 | xxxx | 1229 |
| ATOM | 1230 | N | THR | A | 158 | 29.976 | 9.320 | 74.421 | 1.00 | 0.00 | xxxx | 1230 |
| ATOM | 1231 | CA | THR | A | 158 | 29.191 | 8.785 | 73.315 | 1.00 | 0.00 | xxxx | 1231 |
| ATOM | 1232 | C | THR | A | 158 | 29.959 | 7.686 | 72.595 | 1.00 | 0.00 | xxxx | 1232 |
| ATOM | 1233 | O | THR | A | 158 | 29.416 | 6.605 | 72.330 | 1.00 | 0.00 | xxxx | 1233 |
| ATOM | 1234 | CB | THR | A | 158 | 28.833 | 9.930 | 72.372 | 1.00 | 0.00 | xxxx | 1234 |
| ATOM | 1235 | OG1 | THR | A | 158 | 28.018 | 10.882 | 73.082 | 1.00 | 0.00 | xxxx | 1235 |
| ATOM | 1236 | CG2 | THR | A | 158 | 28.062 | 9.415 | 71.167 | 1.00 | 0.00 | xxxx | 1236 |
| ATOM | 1237 | N | GLN | A | 159 | 31.233 | 7.940 | 72.272 | 1.00 | 0.00 | xxxx | 1237 |
| ATOM | 1238 | CA | GLN | A | 159 | 32.036 | 6.955 | 71.558 | 1.00 | 0.00 | xxxx | 1238 |
| ATOM | 1239 | C | GLN | A | 159 | 32.249 | 5.703 | 72.401 | 1.00 | 0.00 | xxxx | 1239 |
| ATOM | 1240 | O | GLN | A | 159 | 31.998 | 4.580 | 71.945 | 1.00 | 0.00 | xxxx | 1240 |
| ATOM | 1241 | CB | GLN | A | 159 | 33.383 | 7.574 | 71.176 | 1.00 | 0.00 | xxxx | 1241 |
| ATOM | 1242 | CG | GLN | A | 159 | 34.323 | 6.608 | 70.470 | 1.00 | 0.00 | xxxx | 1242 |
| ATOM | 1243 | CD | GLN | A | 159 | 35.726 | 7.162 | 70.282 | 1.00 | 0.00 | xxxx | 1243 |
| ATOM | 1244 | OE1 | GLN | A | 159 | 36.606 | 6.481 | 69.755 | 1.00 | 0.00 | xxxx | 1244 |
| ATOM | 1245 | NE2 | GLN | A | 159 | 35.940 | 8.397 | 70.714 | 1.00 | 0.00 | xxxx | 1245 |
| ATOM | 1246 | N | TYR | A | 160 | 32.726 | 5.877 | 73.632 | 1.00 | 0.00 | xxxx | 1246 |
| ATOM | 1247 | CA | TYR | A | 160 | 33.216 | 4.739 | 74.401 | 1.00 | 0.00 | xxxx | 1247 |
| ATOM | 1248 | C | TYR | A | 160 | 32.105 | 3.875 | 74.979 | 1.00 | 0.00 | xxxx | 1248 |
| ATOM | 1249 | O | TYR | A | 160 | 32.308 | 2.669 | 75.158 | 1.00 | 0.00 | xxxx | 1249 |
| ATOM | 1250 | CB | TYR | A | 160 | 34.204 | 5.198 | 75.472 | 1.00 | 0.00 | xxxx | 1250 |
| ATOM | 1251 | CG | TYR | A | 160 | 35.601 | 5.391 | 74.914 | 1.00 | 0.00 | xxxx | 1251 |
| ATOM | 1252 | CD1 | TYR | A | 160 | 35.946 | 6.552 | 74.224 | 1.00 | 0.00 | xxxx | 1252 |
| ATOM | 1253 | CD2 | TYR | A | 160 | 36.569 | 4.399 | 75.043 | 1.00 | 0.00 | xxxx | 1253 |
| ATOM | 1254 | CE1 | TYR | A | 160 | 37.221 | 6.732 | 73.701 | 1.00 | 0.00 | xxxx | 1254 |
| ATOM | 1255 | CE2 | TYR | A | 160 | 37.844 | 4.572 | 74.517 | 1.00 | 0.00 | xxxx | 1255 |
| ATOM | 1256 | CZ | TYR | A | 160 | 38.156 | 5.739 | 73.849 | 1.00 | 0.00 | xxxx | 1256 |
| ATOM | 1257 | OH | TYR | A | 160 | 39.427 | 5.902 | 73.335 | 1.00 | 0.00 | xxxx | 1257 |
| ATOM | 1258 | N | SER | A | 161 | 30.942 | 4.453 | 75.289 | 1.00 | 0.00 | xxxx | 1258 |
| ATOM | 1259 | CA | SER | A | 161 | 29.842 | 3.626 | 75.783 | 1.00 | 0.00 | xxxx | 1259 |
| ATOM | 1260 | C | SER | A | 161 | 29.450 | 2.579 | 74.750 | 1.00 | 0.00 | xxxx | 1260 |
| ATOM | 1261 | O | SER | A | 161 | 29.409 | 1.380 | 75.045 | 1.00 | 0.00 | xxxx | 1261 |
| ATOM | 1262 | CB | SER | A | 161 | 28.635 | 4.486 | 76.172 | 1.00 | 0.00 | xxxx | 1262 |
| ATOM | 1263 | OG | SER | A | 161 | 28.180 | 5.293 | 75.088 | 1.00 | 0.00 | xxxx | 1263 |
| ATOM | 1264 | N | ILE | A | 162 | 29.211 | 3.013 | 73.512 | 1.00 | 0.00 | xxxx | 1264 |
| ATOM | 1265 | CA | ILE | A | 162 | 28.751 | 2.089 | 72.486 | 1.00 | 0.00 | xxxx | 1265 |
| ATOM | 1266 | C | ILE | A | 162 | 29.858 | 1.119 | 72.084 | 1.00 | 0.00 | xxxx | 1266 |
| ATOM | 1267 | O | ILE | A | 162 | 29.612 | −0.081 | 71.896 | 1.00 | 0.00 | xxxx | 1267 |
| ATOM | 1268 | CB | ILE | A | 162 | 28.164 | 2.856 | 71.291 | 1.00 | 0.00 | xxxx | 1268 |
| ATOM | 1269 | CG1 | ILE | A | 162 | 26.955 | 3.692 | 71.732 | 1.00 | 0.00 | xxxx | 1269 |
| ATOM | 1270 | CG2 | ILE | A | 162 | 27.772 | 1.878 | 70.193 | 1.00 | 0.00 | xxxx | 1270 |
| ATOM | 1271 | CD1 | ILE | A | 162 | 25.902 | 2.898 | 72.512 | 1.00 | 0.00 | xxxx | 1271 |
| ATOM | 1272 | N | GLN | A | 163 | 31.097 | 1.609 | 71.946 | 1.00 | 0.00 | xxxx | 1272 |
| ATOM | 1273 | CA | GLN | A | 163 | 32.182 | 0.701 | 71.592 | 1.00 | 0.00 | xxxx | 1273 |
| ATOM | 1274 | C | GLN | A | 163 | 32.360 | −0.382 | 72.651 | 1.00 | 0.00 | xxxx | 1274 |
| ATOM | 1275 | O | GLN | A | 163 | 32.675 | −1.531 | 72.318 | 1.00 | 0.00 | xxxx | 1275 |
| ATOM | 1276 | CB | GLN | A | 163 | 33.485 | 1.469 | 71.379 | 1.00 | 0.00 | xxxx | 1276 |
| ATOM | 1277 | CG | GLN | A | 163 | 34.589 | 0.592 | 70.798 | 1.00 | 0.00 | xxxx | 1277 |
| ATOM | 1278 | CD | GLN | A | 163 | 34.210 | 0.021 | 69.443 | 1.00 | 0.00 | xxxx | 1278 |
| ATOM | 1279 | OE1 | GLN | A | 163 | 33.819 | 0.760 | 68.534 | 1.00 | 0.00 | xxxx | 1279 |
| ATOM | 1280 | NE2 | GLN | A | 163 | 34.325 | −1.297 | 69.299 | 1.00 | 0.00 | xxxx | 1280 |
| ATOM | 1281 | N | THR | A | 164 | 32.138 | −0.042 | 73.924 | 1.00 | 0.00 | xxxx | 1281 |
| ATOM | 1282 | CA | THR | A | 164 | 32.266 | −1.035 | 74.989 | 1.00 | 0.00 | xxxx | 1282 |
| ATOM | 1283 | C | THR | A | 164 | 31.188 | −2.114 | 74.887 | 1.00 | 0.00 | xxxx | 1283 |
| ATOM | 1284 | O | THR | A | 164 | 31.462 | −3.298 | 75.115 | 1.00 | 0.00 | xxxx | 1284 |
| ATOM | 1285 | CB | THR | A | 164 | 32.231 | −0.326 | 76.339 | 1.00 | 0.00 | xxxx | 1285 |
| ATOM | 1286 | OG1 | THR | A | 164 | 33.385 | 0.516 | 76.443 | 1.00 | 0.00 | xxxx | 1286 |
| ATOM | 1287 | CG2 | THR | A | 164 | 32.192 | −1.322 | 77.498 | 1.00 | 0.00 | xxxx | 1287 |
| ATOM | 1288 | N | VAL | A | 165 | 29.962 | −1.726 | 74.523 | 1.00 | 0.00 | xxxx | 1288 |
| ATOM | 1289 | CA | VAL | A | 165 | 28.908 | −2.715 | 74.291 | 1.00 | 0.00 | xxxx | 1289 |
| ATOM | 1290 | C | VAL | A | 165 | 29.292 | −3.652 | 73.148 | 1.00 | 0.00 | xxxx | 1290 |
| ATOM | 1291 | O | VAL | A | 165 | 29.170 | −4.881 | 73.256 | 1.00 | 0.00 | xxxx | 1291 |
| ATOM | 1292 | CB | VAL | A | 165 | 27.558 | −2.016 | 74.045 | 1.00 | 0.00 | xxxx | 1292 |
| ATOM | 1293 | CG1 | VAL | A | 165 | 26.507 | −3.043 | 73.702 | 1.00 | 0.00 | xxxx | 1293 |
| ATOM | 1294 | CG2 | VAL | A | 165 | 27.136 | −1.217 | 75.278 | 1.00 | 0.00 | xxxx | 1294 |
| ATOM | 1295 | N | LYS | A | 166 | 29.769 | −3.081 | 72.031 | 1.00 | 0.00 | xxxx | 1295 |
| ATOM | 1296 | CA | LYS | A | 166 | 30.209 | −3.908 | 70.910 | 1.00 | 0.00 | xxxx | 1296 |
| ATOM | 1297 | C | LYS | A | 166 | 31.347 | −4.835 | 71.321 | 1.00 | 0.00 | xxxx | 1297 |
| ATOM | 1298 | O | LYS | A | 166 | 31.358 | −6.019 | 70.956 | 1.00 | 0.00 | xxxx | 1298 |
| ATOM | 1299 | CB | LYS | A | 166 | 30.642 | −3.042 | 69.724 | 1.00 | 0.00 | xxxx | 1299 |
| ATOM | 1300 | CG | LYS | A | 166 | 29.518 | −2.242 | 69.057 | 1.00 | 0.00 | xxxx | 1300 |
| ATOM | 1301 | CD | LYS | A | 166 | 30.103 | −1.408 | 67.921 | 1.00 | 0.00 | xxxx | 1301 |
| ATOM | 1302 | CE | LYS | A | 166 | 29.086 | −0.449 | 67.330 | 1.00 | 0.00 | xxxx | 1302 |
| ATOM | 1303 | NZ | LYS | A | 166 | 29.719 | 0.434 | 66.305 | 1.00 | 0.00 | xxxx | 1303 |
| ATOM | 1304 | N | ASP | A | 167 | 32.314 | −4.317 | 72.091 | 1.00 | 0.00 | xxxx | 1304 |
| ATOM | 1305 | CA | ASP | A | 167 | 33.450 | −5.133 | 72.516 | 1.00 | 0.00 | xxxx | 1305 |
| ATOM | 1306 | C | ASP | A | 167 | 33.025 | −6.249 | 73.457 | 1.00 | 0.00 | xxxx | 1306 |
| ATOM | 1307 | O | ASP | A | 167 | 33.751 | −7.243 | 73.593 | 1.00 | 0.00 | xxxx | 1307 |
| ATOM | 1308 | CB | ASP | A | 167 | 34.501 | −4.266 | 73.205 | 1.00 | 0.00 | xxxx | 1308 |
| ATOM | 1309 | CG | ASP | A | 167 | 35.185 | −3.296 | 72.251 | 1.00 | 0.00 | xxxx | 1309 |
| ATOM | 1310 | OD1 | ASP | A | 167 | 35.057 | −3.472 | 71.021 | 1.00 | 0.00 | xxxx | 1310 |
| ATOM | 1311 | OD2 | ASP | A | 167 | 35.853 | −2.359 | 72.741 | 1.00 | 0.00 | xxxx | 1311 |
| ATOM | 1312 | N | ALA | A | 168 | 31.877 | −6.097 | 74.113 | 1.00 | 0.00 | xxxx | 1312 |
| ATOM | 1313 | CA | ALA | A | 168 | 31.300 | −7.137 | 74.956 | 1.00 | 0.00 | xxxx | 1313 |
| ATOM | 1314 | C | ALA | A | 168 | 30.633 | −8.245 | 74.151 | 1.00 | 0.00 | xxxx | 1314 |
| ATOM | 1315 | O | ALA | A | 168 | 30.095 | −9.183 | 74.748 | 1.00 | 0.00 | xxxx | 1315 |
| ATOM | 1316 | CB | ALA | A | 168 | 30.296 | −6.524 | 75.940 | 1.00 | 0.00 | xxxx | 1316 |
| ATOM | 1317 | N | GLY | A | 169 | 30.670 | −8.168 | 72.823 | 1.00 | 0.00 | xxxx | 1317 |
| ATOM | 1318 | CA | GLY | A | 169 | 30.091 | −9.188 | 71.970 | 1.00 | 0.00 | xxxx | 1318 |
| ATOM | 1319 | C | GLY | A | 169 | 28.645 | −8.970 | 71.596 | 1.00 | 0.00 | xxxx | 1319 |
| ATOM | 1320 | O | GLY | A | 169 | 28.017 | −9.884 | 71.050 | 1.00 | 0.00 | xxxx | 1320 |
| ATOM | 1321 | N | ILE | A | 170 | 28.099 | −7.792 | 71.854 | 1.00 | 0.00 | xxxx | 1321 |
| ATOM | 1322 | CA | ILE | A | 170 | 26.694 | −7.506 | 71.609 | 1.00 | 0.00 | xxxx | 1322 |
| ATOM | 1323 | C | ILE | A | 170 | 26.583 | −6.703 | 70.322 | 1.00 | 0.00 | xxxx | 1323 |
| ATOM | 1324 | O | ILE | A | 170 | 27.296 | −5.707 | 70.139 | 1.00 | 0.00 | xxxx | 1324 |
| ATOM | 1325 | CB | ILE | A | 170 | 26.093 | −6.735 | 72.795 | 1.00 | 0.00 | xxxx | 1325 |
| ATOM | 1326 | CG1 | ILE | A | 170 | 26.183 | −7.579 | 74.066 | 1.00 | 0.00 | xxxx | 1326 |
| ATOM | 1327 | CG2 | ILE | A | 170 | 24.659 | −6.295 | 72.475 | 1.00 | 0.00 | xxxx | 1327 |
| ATOM | 1328 | CD1 | ILE | A | 170 | 26.067 | −6.777 | 75.344 | 1.00 | 0.00 | xxxx | 1328 |
| ATOM | 1329 | N | LYS | A | 171 | 25.697 | −7.136 | 69.425 | 1.00 | 0.00 | xxxx | 1329 |
| ATOM | 1330 | CA | LYS | A | 171 | 25.391 | −6.374 | 68.220 | 1.00 | 0.00 | xxxx | 1330 |
| ATOM | 1331 | C | LYS | A | 171 | 24.407 | −5.264 | 68.570 | 1.00 | 0.00 | xxxx | 1331 |
| ATOM | 1332 | O | LYS | A | 171 | 23.503 | −5.464 | 69.384 | 1.00 | 0.00 | xxxx | 1332 |
| ATOM | 1333 | CB | LYS | A | 171 | 24.764 | −7.289 | 67.167 | 1.00 | 0.00 | xxxx | 1333 |
| ATOM | 1334 | CG | LYS | A | 171 | 25.686 | −8.416 | 66.699 | 1.00 | 0.00 | xxxx | 1334 |
| ATOM | 1335 | CD | LYS | A | 171 | 24.971 | −9.366 | 65.729 | 1.00 | 0.00 | xxxx | 1335 |
| ATOM | 1336 | CE | LYS | A | 171 | 25.825 | −10.588 | 65.435 | 1.00 | 0.00 | xxxx | 1336 |
| ATOM | 1337 | NZ | LYS | A | 171 | 25.031 | −11.698 | 64.837 | 1.00 | 0.00 | xxxx | 1337 |
| ATOM | 1338 | N | VAL | A | 172 | 24.593 | −4.083 | 67.966 | 1.00 | 0.00 | xxxx | 1338 |
| ATOM | 1339 | CA | VAL | A | 172 | 23.787 | −2.912 | 68.297 | 1.00 | 0.00 | xxxx | 1339 |
| ATOM | 1340 | C | VAL | A | 172 | 23.218 | −2.271 | 67.036 | 1.00 | 0.00 | xxxx | 1340 |
| ATOM | 1341 | O | VAL | A | 172 | 23.733 | −2.440 | 65.927 | 1.00 | 0.00 | xxxx | 1341 |
| ATOM | 1342 | CB | VAL | A | 172 | 24.574 | −1.866 | 69.114 | 1.00 | 0.00 | xxxx | 1342 |
| ATOM | 1343 | CG1 | VAL | A | 172 | 25.130 | −2.494 | 70.391 | 1.00 | 0.00 | xxxx | 1343 |
| ATOM | 1344 | CG2 | VAL | A | 172 | 25.686 | −1.255 | 68.279 | 1.00 | 0.00 | xxxx | 1344 |
| ATOM | 1345 | N | GLN | A | 173 | 22.145 | −1.498 | 67.226 | 1.00 | 0.00 | xxxx | 1345 |
| ATOM | 1346 | CA | GLN | A | 173 | 21.547 | −0.710 | 66.155 | 1.00 | 0.00 | xxxx | 1346 |
| ATOM | 1347 | C | GLN | A | 173 | 21.113 | 0.620 | 66.741 | 1.00 | 0.00 | xxxx | 1347 |
| ATOM | 1348 | O | GLN | A | 173 | 20.268 | 0.654 | 67.635 | 1.00 | 0.00 | xxxx | 1348 |
| ATOM | 1349 | CB | GLN | A | 173 | 20.343 | −1.417 | 65.536 | 1.00 | 0.00 | xxxx | 1349 |
| ATOM | 1350 | CG | GLN | A | 173 | 19.716 | −0.603 | 64.411 | 1.00 | 0.00 | xxxx | 1350 |
| ATOM | 1351 | CD | GLN | A | 173 | 18.550 | −1.322 | 63.755 | 1.00 | 0.00 | xxxx | 1351 |
| ATOM | 1352 | OE1 | GLN | A | 173 | 17.694 | −1.888 | 64.437 | 1.00 | 0.00 | xxxx | 1352 |
| ATOM | 1353 | NE2 | GLN | A | 173 | 18.504 | −1.298 | 62.422 | 1.00 | 0.00 | xxxx | 1353 |
| ATOM | 1354 | N | GLU | A | 174 | 21.667 | 1.708 | 66.216 | 1.00 | 0.00 | xxxx | 1354 |
| ATOM | 1355 | CA | GLU | A | 174 | 21.374 | 3.052 | 66.698 | 1.00 | 0.00 | xxxx | 1355 |
| ATOM | 1356 | C | GLU | A | 174 | 20.115 | 3.563 | 66.003 | 1.00 | 0.00 | xxxx | 1356 |
| ATOM | 1357 | O | GLU | A | 174 | 20.152 | 3.932 | 64.825 | 1.00 | 0.00 | xxxx | 1357 |
| ATOM | 1358 | CB | GLU | A | 174 | 22.555 | 3.952 | 66.369 | 1.00 | 0.00 | xxxx | 1358 |
| ATOM | 1359 | CG | GLU | A | 174 | 22.459 | 5.336 | 66.957 | 1.00 | 0.00 | xxxx | 1359 |
| ATOM | 1360 | CD | GLU | A | 174 | 23.746 | 6.107 | 66.755 | 1.00 | 0.00 | xxxx | 1360 |
| ATOM | 1361 | OE1 | GLU | A | 174 | 23.782 | 6.964 | 65.861 | 1.00 | 0.00 | xxxx | 1361 |
| ATOM | 1362 | OE2 | GLU | A | 174 | 24.724 | 5.832 | 67.487 | 1.00 | 0.00 | xxxx | 1362 |
| ATOM | 1363 | N | LEU | A | 175 | 19.000 | 3.589 | 66.736 | 1.00 | 0.00 | xxxx | 1363 |
| ATOM | 1364 | CA | LEU | A | 175 | 17.750 | 4.086 | 66.168 | 1.00 | 0.00 | xxxx | 1364 |
| ATOM | 1365 | C | LEU | A | 175 | 17.743 | 5.599 | 66.073 | 1.00 | 0.00 | xxxx | 1365 |
| ATOM | 1366 | O | LEU | A | 175 | 17.143 | 6.155 | 65.145 | 1.00 | 0.00 | xxxx | 1366 |
| ATOM | 1367 | CB | LEU | A | 175 | 16.565 | 3.645 | 67.029 | 1.00 | 0.00 | xxxx | 1367 |
| ATOM | 1368 | CG | LEU | A | 175 | 16.264 | 2.145 | 67.095 | 1.00 | 0.00 | xxxx | 1368 |
| ATOM | 1369 | CD1 | LEU | A | 175 | 15.106 | 1.900 | 68.076 | 1.00 | 0.00 | xxxx | 1369 |
| ATOM | 1370 | CD2 | LEU | A | 175 | 15.921 | 1.605 | 65.723 | 1.00 | 0.00 | xxxx | 1370 |
| ATOM | 1371 | N | ALA | A | 176 | 18.385 | 6.272 | 67.022 | 1.00 | 0.00 | xxxx | 1371 |
| ATOM | 1372 | CA | ALA | A | 176 | 18.405 | 7.726 | 67.039 | 1.00 | 0.00 | xxxx | 1372 |
| ATOM | 1373 | C | ALA | A | 176 | 19.520 | 8.206 | 67.955 | 1.00 | 0.00 | xxxx | 1373 |
| ATOM | 1374 | O | ALA | A | 176 | 19.854 | 7.567 | 68.956 | 1.00 | 0.00 | xxxx | 1374 |
| ATOM | 1375 | CB | ALA | A | 176 | 17.061 | 8.289 | 67.522 | 1.00 | 0.00 | xxxx | 1375 |
| ATOM | 1376 | N | LYS | A | 177 | 20.073 | 9.361 | 67.610 | 1.00 | 0.00 | xxxx | 1376 |
| ATOM | 1377 | CA | LYS | A | 177 | 21.068 | 10.012 | 68.447 | 1.00 | 0.00 | xxxx | 1377 |
| ATOM | 1378 | C | LYS | A | 177 | 20.969 | 11.508 | 68.191 | 1.00 | 0.00 | xxxx | 1378 |
| ATOM | 1379 | O | LYS | A | 177 | 20.868 | 11.934 | 67.036 | 1.00 | 0.00 | xxxx | 1379 |
| ATOM | 1380 | CB | LYS | A | 177 | 22.451 | 9.486 | 68.063 | 1.00 | 0.00 | xxxx | 1380 |
| ATOM | 1381 | CG | LYS | A | 177 | 23.620 | 10.280 | 68.569 | 1.00 | 0.00 | xxxx | 1381 |
| ATOM | 1382 | CD | LYS | A | 177 | 24.936 | 9.671 | 68.080 | 1.00 | 0.00 | xxxx | 1382 |
| ATOM | 1383 | CE | LYS | A | 177 | 25.116 | 9.859 | 66.580 | 1.00 | 0.00 | xxxx | 1383 |
| ATOM | 1384 | NZ | LYS | A | 177 | 26.421 | 9.325 | 66.103 | 1.00 | 0.00 | xxxx | 1384 |
| ATOM | 1385 | N | ASP | A | 178 | 20.972 | 12.305 | 69.256 | 1.00 | 0.00 | xxxx | 1385 |
| ATOM | 1386 | CA | ASP | A | 178 | 20.891 | 13.751 | 69.083 | 1.00 | 0.00 | xxxx | 1386 |
| ATOM | 1387 | C | ASP | A | 178 | 21.464 | 14.426 | 70.321 | 1.00 | 0.00 | xxxx | 1387 |
| ATOM | 1388 | O | ASP | A | 178 | 21.598 | 13.806 | 71.386 | 1.00 | 0.00 | xxxx | 1388 |
| ATOM | 1389 | CB | ASP | A | 178 | 19.438 | 14.192 | 68.859 | 1.00 | 0.00 | xxxx | 1389 |
| ATOM | 1390 | CG | ASP | A | 178 | 19.325 | 15.526 | 68.130 | 1.00 | 0.00 | xxxx | 1390 |
| ATOM | 1391 | OD1 | ASP | A | 178 | 20.358 | 16.190 | 67.860 | 1.00 | 0.00 | xxxx | 1391 |
| ATOM | 1392 | OD2 | ASP | A | 178 | 18.171 | 15.921 | 67.840 | 1.00 | 0.00 | xxxx | 1392 |
| ATOM | 1393 | N | TYR | A | 179 | 21.795 | 15.709 | 70.170 | 1.00 | 0.00 | xxxx | 1393 |
| ATOM | 1394 | CA | TYR | A | 179 | 22.231 | 16.525 | 71.294 | 1.00 | 0.00 | xxxx | 1394 |
| ATOM | 1395 | C | TYR | A | 179 | 21.036 | 17.016 | 72.102 | 1.00 | 0.00 | xxxx | 1395 |
| ATOM | 1396 | O | TYR | A | 179 | 20.065 | 17.544 | 71.543 | 1.00 | 0.00 | xxxx | 1396 |
| ATOM | 1397 | CB | TYR | A | 179 | 22.997 | 17.731 | 70.768 | 1.00 | 0.00 | xxxx | 1397 |
| ATOM | 1398 | CG | TYR | A | 179 | 24.266 | 17.368 | 70.052 | 1.00 | 0.00 | xxxx | 1398 |
| ATOM | 1399 | CD1 | TYR | A | 179 | 25.388 | 16.951 | 70.763 | 1.00 | 0.00 | xxxx | 1399 |
| ATOM | 1400 | CD2 | TYR | A | 179 | 24.364 | 17.476 | 68.668 | 1.00 | 0.00 | xxxx | 1400 |
| ATOM | 1401 | CE1 | TYR | A | 179 | 26.562 | 16.628 | 70.123 | 1.00 | 0.00 | xxxx | 1401 |
| ATOM | 1402 | CE2 | TYR | A | 179 | 25.540 | 17.153 | 68.015 | 1.00 | 0.00 | xxxx | 1402 |
| ATOM | 1403 | CZ | TYR | A | 179 | 26.634 | 16.732 | 68.753 | 1.00 | 0.00 | xxxx | 1403 |
| ATOM | 1404 | OH | TYR | A | 179 | 27.818 | 16.416 | 68.129 | 1.00 | 0.00 | xxxx | 1404 |
| ATOM | 1405 | N | ALA | A | 180 | 21.129 | 16.886 | 73.425 | 1.00 | 0.00 | xxxx | 1405 |
| ATOM | 1406 | CA | ALA | A | 180 | 20.161 | 17.491 | 74.341 | 1.00 | 0.00 | xxxx | 1406 |
| ATOM | 1407 | C | ALA | A | 180 | 20.827 | 18.427 | 75.349 | 1.00 | 0.00 | xxxx | 1407 |
| ATOM | 1408 | O | ALA | A | 180 | 20.185 | 18.846 | 76.324 | 1.00 | 0.00 | xxxx | 1408 |
| ATOM | 1409 | CB | ALA | A | 180 | 19.282 | 16.432 | 75.019 | 1.00 | 0.00 | xxxx | 1409 |
| ATOM | 1410 | N | ASN | A | 181 | 22.103 | 18.760 | 75.148 | 1.00 | 0.00 | xxxx | 1410 |
| ATOM | 1411 | CA | ASN | A | 181 | 22.741 | 19.891 | 75.828 | 1.00 | 0.00 | xxxx | 1411 |
| ATOM | 1412 | C | ASN | A | 181 | 22.737 | 19.750 | 77.348 | 1.00 | 0.00 | xxxx | 1412 |
| ATOM | 1413 | O | ASN | A | 181 | 22.585 | 20.733 | 78.075 | 1.00 | 0.00 | xxxx | 1413 |
| ATOM | 1414 | CB | ASN | A | 181 | 22.109 | 21.218 | 75.410 | 1.00 | 0.00 | xxxx | 1414 |
| ATOM | 1415 | CG | ASN | A | 181 | 22.101 | 21.407 | 73.914 | 1.00 | 0.00 | xxxx | 1415 |
| ATOM | 1416 | OD1 | ASN | A | 181 | 23.098 | 21.152 | 73.242 | 1.00 | 0.00 | xxxx | 1416 |
| ATOM | 1417 | ND2 | ASN | A | 181 | 20.967 | 21.841 | 73.379 | 1.00 | 0.00 | xxxx | 1417 |
| ATOM | 1418 | N | CYS | A | 182 | 22.892 | 18.522 | 77.836 | 1.00 | 0.00 | xxxx | 1418 |
| ATOM | 1419 | CA | CYS | A | 182 | 23.033 | 18.226 | 79.262 | 1.00 | 0.00 | xxxx | 1419 |
| ATOM | 1420 | C | CYS | A | 182 | 21.808 | 18.592 | 80.072 | 1.00 | 0.00 | xxxx | 1420 |
| ATOM | 1421 | O | CYS | A | 182 | 21.891 | 18.631 | 81.308 | 1.00 | 0.00 | xxxx | 1421 |
| ATOM | 1422 | CB | CYS | A | 182 | 24.251 | 18.909 | 79.885 | 1.00 | 0.00 | xxxx | 1422 |
| ATOM | 1423 | SG | CYS | A | 182 | 25.697 | 18.733 | 78.864 | 1.00 | 0.00 | xxxx | 1423 |
| ATOM | 1424 | N | ASP | A | 183 | 20.679 | 18.850 | 79.417 | 1.00 | 0.00 | xxxx | 1424 |
| ATOM | 1425 | CA | ASP | A | 183 | 19.525 | 19.500 | 80.025 | 1.00 | 0.00 | xxxx | 1425 |
| ATOM | 1426 | C | ASP | A | 183 | 18.357 | 18.525 | 80.123 | 1.00 | 0.00 | xxxx | 1426 |
| ATOM | 1427 | O | ASP | A | 183 | 18.018 | 17.854 | 79.145 | 1.00 | 0.00 | xxxx | 1427 |
| ATOM | 1428 | CB | ASP | A | 183 | 19.134 | 20.721 | 79.186 | 1.00 | 0.00 | xxxx | 1428 |
| ATOM | 1429 | CG | ASP | A | 183 | 17.898 | 21.418 | 79.700 | 1.00 | 0.00 | xxxx | 1429 |
| ATOM | 1430 | OD1 | ASP | A | 183 | 17.964 | 22.029 | 80.787 | 1.00 | 0.00 | xxxx | 1430 |
| ATOM | 1431 | OD2 | ASP | A | 183 | 16.865 | 21.371 | 79.002 | 1.00 | 0.00 | xxxx | 1431 |
| ATOM | 1432 | N | ARG | A | 184 | 17.722 | 18.489 | 81.299 | 1.00 | 0.00 | xxxx | 1432 |
| ATOM | 1433 | CA | ARG | A | 184 | 16.621 | 17.559 | 81.545 | 1.00 | 0.00 | xxxx | 1433 |
| ATOM | 1434 | C | ARG | A | 184 | 15.435 | 17.809 | 80.611 | 1.00 | 0.00 | xxxx | 1434 |
| ATOM | 1435 | O | ARG | A | 184 | 14.903 | 16.872 | 80.007 | 1.00 | 0.00 | xxxx | 1435 |
| ATOM | 1436 | CB | ARG | A | 184 | 16.204 | 17.645 | 83.015 | 1.00 | 0.00 | xxxx | 1436 |
| ATOM | 1437 | CG | ARG | A | 184 | 15.048 | 16.728 | 83.405 | 1.00 | 0.00 | xxxx | 1437 |
| ATOM | 1438 | CD | ARG | A | 184 | 14.741 | 16.814 | 84.892 | 1.00 | 0.00 | xxxx | 1438 |
| ATOM | 1439 | NE | ARG | A | 184 | 14.478 | 18.190 | 85.306 | 1.00 | 0.00 | xxxx | 1439 |
| ATOM | 1440 | CZ | ARG | A | 184 | 13.315 | 18.812 | 85.142 | 1.00 | 0.00 | xxxx | 1440 |
| ATOM | 1441 | NH1 | ARG | A | 184 | 12.298 | 18.184 | 84.566 | 1.00 | 0.00 | xxxx | 1441 |
| ATOM | 1442 | NH2 | ARG | A | 184 | 13.168 | 20.067 | 85.551 | 1.00 | 0.00 | xxxx | 1442 |
| ATOM | 1443 | N | VAL | A | 185 | 15.000 | 19.066 | 80.470 | 1.00 | 0.00 | xxxx | 1443 |
| ATOM | 1444 | CA | VAL | A | 185 | 13.800 | 19.336 | 79.668 | 1.00 | 0.00 | xxxx | 1444 |
| ATOM | 1445 | C | VAL | A | 185 | 14.028 | 19.039 | 78.183 | 1.00 | 0.00 | xxxx | 1445 |
| ATOM | 1446 | O | VAL | A | 185 | 13.175 | 18.443 | 77.516 | 1.00 | 0.00 | xxxx | 1446 |
| ATOM | 1447 | CB | VAL | A | 185 | 13.286 | 20.764 | 79.905 | 1.00 | 0.00 | xxxx | 1447 |
| ATOM | 1448 | CG1 | VAL | A | 185 | 12.201 | 21.110 | 78.884 | 1.00 | 0.00 | xxxx | 1448 |
| ATOM | 1449 | CG2 | VAL | A | 185 | 12.752 | 20.892 | 81.327 | 1.00 | 0.00 | xxxx | 1449 |
| ATOM | 1450 | N | THR | A | 186 | 15.168 | 19.461 | 77.633 | 1.00 | 0.00 | xxxx | 1450 |
| ATOM | 1451 | CA | THR | A | 186 | 15.460 | 19.147 | 76.237 | 1.00 | 0.00 | xxxx | 1451 |
| ATOM | 1452 | C | THR | A | 186 | 15.495 | 17.640 | 76.014 | 1.00 | 0.00 | xxxx | 1452 |
| ATOM | 1453 | O | THR | A | 186 | 14.978 | 17.136 | 75.006 | 1.00 | 0.00 | xxxx | 1453 |
| ATOM | 1454 | CB | THR | A | 186 | 16.810 | 19.741 | 75.852 | 1.00 | 0.00 | xxxx | 1454 |
| ATOM | 1455 | OG1 | THR | A | 186 | 16.814 | 21.143 | 76.153 | 1.00 | 0.00 | xxxx | 1455 |
| ATOM | 1456 | CG2 | THR | A | 186 | 17.068 | 19.526 | 74.365 | 1.00 | 0.00 | xxxx | 1456 |
| ATOM | 1457 | N | ALA | A | 187 | 16.116 | 16.907 | 76.937 | 1.00 | 0.00 | xxxx | 1457 |
| ATOM | 1458 | CA | ALA | A | 187 | 16.173 | 15.456 | 76.812 | 1.00 | 0.00 | xxxx | 1458 |
| ATOM | 1459 | C | ALA | A | 187 | 14.783 | 14.846 | 76.878 | 1.00 | 0.00 | xxxx | 1459 |
| ATOM | 1460 | O | ALA | A | 187 | 14.469 | 13.918 | 76.122 | 1.00 | 0.00 | xxxx | 1460 |
| ATOM | 1461 | CB | ALA | A | 187 | 17.059 | 14.868 | 77.909 | 1.00 | 0.00 | xxxx | 1461 |
| ATOM | 1462 | N | HIS | A | 188 | 13.941 | 15.351 | 77.785 | 1.00 | 0.00 | xxxx | 1462 |
| ATOM | 1463 | CA | HIS | A | 188 | 12.551 | 14.900 | 77.834 | 1.00 | 0.00 | xxxx | 1463 |
| ATOM | 1464 | C | HIS | A | 188 | 11.883 | 15.050 | 76.474 | 1.00 | 0.00 | xxxx | 1464 |
| ATOM | 1465 | O | HIS | A | 188 | 11.220 | 14.127 | 75.989 | 1.00 | 0.00 | xxxx | 1465 |
| ATOM | 1466 | CB | HIS | A | 188 | 11.780 | 15.697 | 78.885 | 1.00 | 0.00 | xxxx | 1466 |
| ATOM | 1467 | CG | HIS | A | 188 | 10.299 | 15.513 | 78.787 | 1.00 | 0.00 | xxxx | 1467 |
| ATOM | 1468 | ND1 | HIS | A | 188 | 9.512 | 16.277 | 77.951 | 1.00 | 0.00 | xxxx | 1468 |
| ATOM | 1469 | CD2 | HIS | A | 188 | 9.467 | 14.632 | 79.390 | 1.00 | 0.00 | xxxx | 1469 |
| ATOM | 1470 | CE1 | HIS | A | 188 | 8.252 | 15.887 | 78.063 | 1.00 | 0.00 | xxxx | 1470 |
| ATOM | 1471 | NE2 | HIS | A | 188 | 8.199 | 14.889 | 78.926 | 1.00 | 0.00 | xxxx | 1471 |
| ATOM | 1472 | N | ASP | A | 189 | 12.049 | 16.221 | 75.846 | 1.00 | 0.00 | xxxx | 1472 |
| ATOM | 1473 | CA | ASP | A | 189 | 11.368 | 16.487 | 74.584 | 1.00 | 0.00 | xxxx | 1473 |
| ATOM | 1474 | C | ASP | A | 189 | 11.903 | 15.599 | 73.467 | 1.00 | 0.00 | xxxx | 1474 |
| ATOM | 1475 | O | ASP | A | 189 | 11.126 | 15.099 | 72.645 | 1.00 | 0.00 | xxxx | 1475 |
| ATOM | 1476 | CB | ASP | A | 189 | 11.479 | 17.973 | 74.226 | 1.00 | 0.00 | xxxx | 1476 |
| ATOM | 1477 | CG | ASP | A | 189 | 10.710 | 18.880 | 75.188 | 1.00 | 0.00 | xxxx | 1477 |
| ATOM | 1478 | OD1 | ASP | A | 189 | 9.954 | 18.372 | 76.049 | 1.00 | 0.00 | xxxx | 1478 |
| ATOM | 1479 | OD2 | ASP | A | 189 | 10.857 | 20.117 | 75.077 | 1.00 | 0.00 | xxxx | 1479 |
| ATOM | 1480 | N | LYS | A | 190 | 13.223 | 15.368 | 73.429 | 1.00 | 0.00 | xxxx | 1480 |
| ATOM | 1481 | CA | LYS | A | 190 | 13.777 | 14.496 | 72.396 | 1.00 | 0.00 | xxxx | 1481 |
| ATOM | 1482 | C | LYS | A | 190 | 13.285 | 13.068 | 72.579 | 1.00 | 0.00 | xxxx | 1482 |
| ATOM | 1483 | O | LYS | A | 190 | 12.891 | 12.408 | 71.606 | 1.00 | 0.00 | xxxx | 1483 |
| ATOM | 1484 | CB | LYS | A | 190 | 15.308 | 14.513 | 72.449 | 1.00 | 0.00 | xxxx | 1484 |
| ATOM | 1485 | CG | LYS | A | 190 | 15.945 | 15.863 | 72.248 | 1.00 | 0.00 | xxxx | 1485 |
| ATOM | 1486 | CD | LYS | A | 190 | 15.977 | 16.260 | 70.788 | 1.00 | 0.00 | xxxx | 1486 |
| ATOM | 1487 | CE | LYS | A | 190 | 16.701 | 17.585 | 70.608 | 1.00 | 0.00 | xxxx | 1487 |
| ATOM | 1488 | NZ | LYS | A | 190 | 16.722 | 17.978 | 69.176 | 1.00 | 0.00 | xxxx | 1488 |
| ATOM | 1489 | N | MET | A | 191 | 13.301 | 12.577 | 73.824 | 1.00 | 0.00 | xxxx | 1489 |
| ATOM | 1490 | CA | MET | A | 191 | 12.870 | 11.208 | 74.093 | 1.00 | 0.00 | xxxx | 1490 |
| ATOM | 1491 | C | MET | A | 191 | 11.392 | 11.025 | 73.776 | 1.00 | 0.00 | xxxx | 1491 |
| ATOM | 1492 | O | MET | A | 191 | 10.990 | 9.992 | 73.224 | 1.00 | 0.00 | xxxx | 1492 |
| ATOM | 1493 | CB | MET | A | 191 | 13.165 | 10.842 | 75.548 | 1.00 | 0.00 | xxxx | 1493 |
| ATOM | 1494 | CG | MET | A | 191 | 12.888 | 9.385 | 75.898 | 1.00 | 0.00 | xxxx | 1494 |
| ATOM | 1495 | SD | MET | A | 191 | 14.209 | 8.341 | 75.237 | 1.00 | 0.00 | xxxx | 1495 |
| ATOM | 1496 | CE | MET | A | 191 | 13.275 | 6.864 | 74.858 | 1.00 | 0.00 | xxxx | 1496 |
| ATOM | 1497 | N | ALA | A | 192 | 10.562 | 12.018 | 74.119 | 1.00 | 0.00 | xxxx | 1497 |
| ATOM | 1498 | CA | ALA | A | 192 | 9.135 | 11.911 | 73.822 | 1.00 | 0.00 | xxxx | 1498 |
| ATOM | 1499 | C | ALA | A | 192 | 8.894 | 11.783 | 72.320 | 1.00 | 0.00 | xxxx | 1499 |
| ATOM | 1500 | O | ALA | A | 192 | 8.039 | 11.002 | 71.882 | 1.00 | 0.00 | xxxx | 1500 |
| ATOM | 1501 | CB | ALA | A | 192 | 8.394 | 13.125 | 74.388 | 1.00 | 0.00 | xxxx | 1501 |
| ATOM | 1502 | N | ALA | A | 193 | 9.640 | 12.543 | 71.517 | 1.00 | 0.00 | xxxx | 1502 |
| ATOM | 1503 | CA | ALA | A | 193 | 9.506 | 12.450 | 70.066 | 1.00 | 0.00 | xxxx | 1503 |
| ATOM | 1504 | C | ALA | A | 193 | 9.962 | 11.087 | 69.559 | 1.00 | 0.00 | xxxx | 1504 |
| ATOM | 1505 | O | ALA | A | 193 | 9.311 | 10.487 | 68.692 | 1.00 | 0.00 | xxxx | 1505 |
| ATOM | 1506 | CB | ALA | A | 193 | 10.276 | 13.595 | 69.410 | 1.00 | 0.00 | xxxx | 1506 |
| ATOM | 1507 | N | TRP | A | 194 | 11.061 | 10.565 | 70.105 | 1.00 | 0.00 | xxxx | 1507 |
| ATOM | 1508 | CA | TRP | A | 194 | 11.526 | 9.246 | 69.692 | 1.00 | 0.00 | xxxx | 1508 |
| ATOM | 1509 | C | TRP | A | 194 | 10.520 | 8.165 | 70.052 | 1.00 | 0.00 | xxxx | 1509 |
| ATOM | 1510 | O | TRP | A | 194 | 10.333 | 7.207 | 69.293 | 1.00 | 0.00 | xxxx | 1510 |
| ATOM | 1511 | CB | TRP | A | 194 | 12.867 | 8.939 | 70.347 | 1.00 | 0.00 | xxxx | 1511 |
| ATOM | 1512 | CG | TRP | A | 194 | 14.000 | 9.749 | 69.833 | 1.00 | 0.00 | xxxx | 1512 |
| ATOM | 1513 | CD1 | TRP | A | 194 | 14.080 | 10.407 | 68.635 | 1.00 | 0.00 | xxxx | 1513 |
| ATOM | 1514 | CD2 | TRP | A | 194 | 15.233 | 9.977 | 70.505 | 1.00 | 0.00 | xxxx | 1514 |
| ATOM | 1515 | NE1 | TRP | A | 194 | 15.297 | 11.041 | 68.533 | 1.00 | 0.00 | xxxx | 1515 |
| ATOM | 1516 | CE2 | TRP | A | 194 | 16.022 | 10.787 | 69.671 | 1.00 | 0.00 | xxxx | 1516 |
| ATOM | 1517 | CE3 | TRP | A | 194 | 15.748 | 9.571 | 71.743 | 1.00 | 0.00 | xxxx | 1517 |
| ATOM | 1518 | CZ2 | TRP | A | 194 | 17.316 | 11.195 | 70.033 | 1.00 | 0.00 | xxxx | 1518 |
| ATOM | 1519 | CZ3 | TRP | A | 194 | 17.023 | 9.974 | 72.104 | 1.00 | 0.00 | xxxx | 1519 |
| ATOM | 1520 | CH2 | TRP | A | 194 | 17.791 | 10.779 | 71.250 | 1.00 | 0.00 | xxxx | 1520 |
| ATOM | 1521 | N | LEU | A | 195 | 9.876 | 8.283 | 71.217 | 1.00 | 0.00 | xxxx | 1521 |
| ATOM | 1522 | CA | LEU | A | 195 | 8.870 | 7.294 | 71.602 | 1.00 | 0.00 | xxxx | 1522 |
| ATOM | 1523 | C | LEU | A | 195 | 7.678 | 7.327 | 70.656 | 1.00 | 0.00 | xxxx | 1523 |
| ATOM | 1524 | O | LEU | A | 195 | 7.104 | 6.280 | 70.327 | 1.00 | 0.00 | xxxx | 1524 |
| ATOM | 1525 | CB | LEU | A | 195 | 8.423 | 7.516 | 73.047 | 1.00 | 0.00 | xxxx | 1525 |
| ATOM | 1526 | CG | LEU | A | 195 | 9.482 | 7.078 | 74.060 | 1.00 | 0.00 | xxxx | 1526 |
| ATOM | 1527 | CD1 | LEU | A | 195 | 9.204 | 7.715 | 75.422 | 1.00 | 0.00 | xxxx | 1527 |
| ATOM | 1528 | CD2 | LEU | A | 195 | 9.505 | 5.553 | 74.169 | 1.00 | 0.00 | xxxx | 1528 |
| ATOM | 1529 | N | SER | A | 196 | 7.294 | 8.521 | 70.208 | 1.00 | 0.00 | xxxx | 1529 |
| ATOM | 1530 | CA | SER | A | 196 | 6.212 | 8.625 | 69.237 | 1.00 | 0.00 | xxxx | 1530 |
| ATOM | 1531 | C | SER | A | 196 | 6.607 | 7.996 | 67.904 | 1.00 | 0.00 | xxxx | 1531 |
| ATOM | 1532 | O | SER | A | 196 | 5.787 | 7.331 | 67.256 | 1.00 | 0.00 | xxxx | 1532 |
| ATOM | 1533 | CB | SER | A | 196 | 5.842 | 10.096 | 69.058 | 1.00 | 0.00 | xxxx | 1533 |
| ATOM | 1534 | OG | SER | A | 196 | 4.793 | 10.240 | 68.117 | 1.00 | 0.00 | xxxx | 1534 |
| ATOM | 1535 | N | SER | A | 197 | 7.867 | 8.170 | 67.498 | 1.00 | 0.00 | xxxx | 1535 |
| ATOM | 1536 | CA | SER | A | 197 | 8.327 | 7.660 | 66.209 | 1.00 | 0.00 | xxxx | 1536 |
| ATOM | 1537 | C | SER | A | 197 | 8.543 | 6.155 | 66.227 | 1.00 | 0.00 | xxxx | 1537 |
| ATOM | 1538 | O | SER | A | 197 | 8.182 | 5.467 | 65.265 | 1.00 | 0.00 | xxxx | 1538 |
| ATOM | 1539 | CB | SER | A | 197 | 9.638 | 8.333 | 65.817 | 1.00 | 0.00 | xxxx | 1539 |
| ATOM | 1540 | OG | SER | A | 197 | 9.428 | 9.672 | 65.436 | 1.00 | 0.00 | xxxx | 1540 |
| ATOM | 1541 | N | PHE | A | 198 | 9.146 | 5.631 | 67.296 | 1.00 | 0.00 | xxxx | 1541 |
| ATOM | 1542 | CA | PHE | A | 198 | 9.667 | 4.272 | 67.310 | 1.00 | 0.00 | xxxx | 1542 |
| ATOM | 1543 | C | PHE | A | 198 | 9.012 | 3.374 | 68.341 | 1.00 | 0.00 | xxxx | 1543 |
| ATOM | 1544 | O | PHE | A | 198 | 9.050 | 2.147 | 68.178 | 1.00 | 0.00 | xxxx | 1544 |
| ATOM | 1545 | CB | PHE | A | 198 | 11.186 | 4.271 | 67.567 | 1.00 | 0.00 | xxxx | 1545 |
| ATOM | 1546 | CG | PHE | A | 198 | 11.980 | 5.046 | 66.550 | 1.00 | 0.00 | xxxx | 1546 |
| ATOM | 1547 | CD1 | PHE | A | 198 | 12.121 | 4.570 | 65.254 | 1.00 | 0.00 | xxxx | 1547 |
| ATOM | 1548 | CD2 | PHE | A | 198 | 12.595 | 6.237 | 66.891 | 1.00 | 0.00 | xxxx | 1548 |
| ATOM | 1549 | CE1 | PHE | A | 198 | 12.854 | 5.280 | 64.315 | 1.00 | 0.00 | xxxx | 1549 |
| ATOM | 1550 | CE2 | PHE | A | 198 | 13.327 | 6.954 | 65.959 | 1.00 | 0.00 | xxxx | 1550 |
| ATOM | 1551 | CZ | PHE | A | 198 | 13.459 | 6.474 | 64.672 | 1.00 | 0.00 | xxxx | 1551 |
| ATOM | 1552 | N | GLY | A | 199 | 8.436 | 3.938 | 69.396 | 1.00 | 0.00 | xxxx | 1552 |
| ATOM | 1553 | CA | GLY | A | 199 | 7.622 | 3.144 | 70.303 | 1.00 | 0.00 | xxxx | 1553 |
| ATOM | 1554 | C | GLY | A | 199 | 8.407 | 2.021 | 70.947 | 1.00 | 0.00 | xxxx | 1554 |
| ATOM | 1555 | O | GLY | A | 199 | 9.526 | 2.204 | 71.447 | 1.00 | 0.00 | xxxx | 1555 |
| ATOM | 1556 | N | ASP | A | 200 | 7.823 | 0.827 | 70.915 | 1.00 | 0.00 | xxxx | 1556 |
| ATOM | 1557 | CA | ASP | A | 200 | 8.417 | −0.299 | 71.617 | 1.00 | 0.00 | xxxx | 1557 |
| ATOM | 1558 | C | ASP | A | 200 | 9.670 | −0.829 | 70.935 | 1.00 | 0.00 | xxxx | 1558 |
| ATOM | 1559 | O | ASP | A | 200 | 10.269 | −1.778 | 71.448 | 1.00 | 0.00 | xxxx | 1559 |
| ATOM | 1560 | CB | ASP | A | 200 | 7.385 | −1.416 | 71.804 | 1.00 | 0.00 | xxxx | 1560 |
| ATOM | 1561 | CG | ASP | A | 200 | 7.738 | −2.355 | 72.951 | 1.00 | 0.00 | xxxx | 1561 |
| ATOM | 1562 | OD1 | ASP | A | 200 | 8.129 | −1.859 | 74.030 | 1.00 | 0.00 | xxxx | 1562 |
| ATOM | 1563 | OD2 | ASP | A | 200 | 7.629 | −3.588 | 72.771 | 1.00 | 0.00 | xxxx | 1563 |
| ATOM | 1564 | N | LYS | A | 201 | 10.100 | −0.242 | 69.813 | 1.00 | 0.00 | xxxx | 1564 |
| ATOM | 1565 | CA | LYS | A | 201 | 11.350 | −0.688 | 69.204 | 1.00 | 0.00 | xxxx | 1565 |
| ATOM | 1566 | C | LYS | A | 201 | 12.558 | −0.358 | 70.073 | 1.00 | 0.00 | xxxx | 1566 |
| ATOM | 1567 | O | LYS | A | 201 | 13.589 | −1.034 | 69.975 | 1.00 | 0.00 | xxxx | 1567 |
| ATOM | 1568 | CB | LYS | A | 201 | 11.546 | −0.053 | 67.826 | 1.00 | 0.00 | xxxx | 1568 |
| ATOM | 1569 | CG | LYS | A | 201 | 10.675 | −0.615 | 66.715 | 1.00 | 0.00 | xxxx | 1569 |
| ATOM | 1570 | CD | LYS | A | 201 | 10.986 | 0.110 | 65.409 | 1.00 | 0.00 | xxxx | 1570 |
| ATOM | 1571 | CE | LYS | A | 201 | 10.412 | −0.603 | 64.198 | 1.00 | 0.00 | xxxx | 1571 |
| ATOM | 1572 | NZ | LYS | A | 201 | 11.042 | −1.941 | 64.005 | 1.00 | 0.00 | xxxx | 1572 |
| ATOM | 1573 | N | ILE | A | 202 | 12.474 | 0.683 | 70.904 | 1.00 | 0.00 | xxxx | 1573 |
| ATOM | 1574 | CA | ILE | A | 202 | 13.634 | 1.098 | 71.688 | 1.00 | 0.00 | xxxx | 1574 |
| ATOM | 1575 | C | ILE | A | 202 | 13.860 | 0.108 | 72.824 | 1.00 | 0.00 | xxxx | 1575 |
| ATOM | 1576 | O | ILE | A | 202 | 12.953 | −0.158 | 73.623 | 1.00 | 0.00 | xxxx | 1576 |
| ATOM | 1577 | CB | ILE | A | 202 | 13.439 | 2.518 | 72.238 | 1.00 | 0.00 | xxxx | 1577 |
| ATOM | 1578 | CG1 | ILE | A | 202 | 13.178 | 3.517 | 71.109 | 1.00 | 0.00 | xxxx | 1578 |
| ATOM | 1579 | CG2 | ILE | A | 202 | 14.660 | 2.953 | 73.069 | 1.00 | 0.00 | xxxx | 1579 |
| ATOM | 1580 | CD1 | ILE | A | 202 | 12.815 | 4.917 | 71.597 | 1.00 | 0.00 | xxxx | 1580 |
| ATOM | 1581 | N | GLU | A | 203 | 15.077 | −0.424 | 72.911 | 1.00 | 0.00 | xxxx | 1581 |
| ATOM | 1582 | CA | GLU | A | 203 | 15.426 | −1.422 | 73.910 | 1.00 | 0.00 | xxxx | 1582 |
| ATOM | 1583 | C | GLU | A | 203 | 16.410 | −0.926 | 74.962 | 1.00 | 0.00 | xxxx | 1583 |
| ATOM | 1584 | O | GLU | A | 203 | 16.580 | −1.596 | 75.992 | 1.00 | 0.00 | xxxx | 1584 |
| ATOM | 1585 | CB | GLU | A | 203 | 15.978 | −2.673 | 73.209 | 1.00 | 0.00 | xxxx | 1585 |
| ATOM | 1586 | CG | GLU | A | 203 | 14.941 | −3.342 | 72.310 | 1.00 | 0.00 | xxxx | 1586 |
| ATOM | 1587 | CD | GLU | A | 203 | 15.505 | −4.464 | 71.460 | 1.00 | 0.00 | xxxx | 1587 |
| ATOM | 1588 | OE1 | GLU | A | 203 | 16.743 | −4.593 | 71.349 | 1.00 | 0.00 | xxxx | 1588 |
| ATOM | 1589 | OE2 | GLU | A | 203 | 14.699 | −5.228 | 70.893 | 1.00 | 0.00 | xxxx | 1589 |
| ATOM | 1590 | N | ALA | A | 204 | 17.064 | 0.210 | 74.739 | 1.00 | 0.00 | xxxx | 1590 |
| ATOM | 1591 | CA | ALA | A | 204 | 18.049 | 0.717 | 75.685 | 1.00 | 0.00 | xxxx | 1591 |
| ATOM | 1592 | C | ALA | A | 204 | 18.211 | 2.210 | 75.445 | 1.00 | 0.00 | xxxx | 1592 |
| ATOM | 1593 | O | ALA | A | 204 | 18.120 | 2.675 | 74.308 | 1.00 | 0.00 | xxxx | 1593 |
| ATOM | 1594 | CB | ALA | A | 204 | 19.400 | 0.028 | 75.483 | 1.00 | 0.00 | xxxx | 1594 |
| ATOM | 1595 | N | VAL | A | 205 | 18.470 | 2.955 | 76.520 | 1.00 | 0.00 | xxxx | 1595 |
| ATOM | 1596 | CA | VAL | A | 205 | 18.690 | 4.399 | 76.447 | 1.00 | 0.00 | xxxx | 1596 |
| ATOM | 1597 | C | VAL | A | 205 | 20.029 | 4.721 | 77.097 | 1.00 | 0.00 | xxxx | 1597 |
| ATOM | 1598 | O | VAL | A | 205 | 20.225 | 4.455 | 78.288 | 1.00 | 0.00 | xxxx | 1598 |
| ATOM | 1599 | CB | VAL | A | 205 | 17.566 | 5.182 | 77.147 | 1.00 | 0.00 | xxxx | 1599 |
| ATOM | 1600 | CG1 | VAL | A | 205 | 17.870 | 6.677 | 77.115 | 1.00 | 0.00 | xxxx | 1600 |
| ATOM | 1601 | CG2 | VAL | A | 205 | 16.203 | 4.854 | 76.517 | 1.00 | 0.00 | xxxx | 1601 |
| ATOM | 1602 | N | PHE | A | 206 | 20.919 | 5.354 | 76.337 | 1.00 | 0.00 | xxxx | 1602 |
| ATOM | 1603 | CA | PHE | A | 206 | 22.159 | 5.899 | 76.870 | 1.00 | 0.00 | xxxx | 1603 |
| ATOM | 1604 | C | PHE | A | 206 | 22.051 | 7.414 | 76.907 | 1.00 | 0.00 | xxxx | 1604 |
| ATOM | 1605 | O | PHE | A | 206 | 21.570 | 8.028 | 75.954 | 1.00 | 0.00 | xxxx | 1605 |
| ATOM | 1606 | CB | PHE | A | 206 | 23.337 | 5.550 | 75.956 | 1.00 | 0.00 | xxxx | 1606 |
| ATOM | 1607 | CG | PHE | A | 206 | 23.627 | 4.061 | 75.837 | 1.00 | 0.00 | xxxx | 1607 |
| ATOM | 1608 | CD1 | PHE | A | 206 | 24.652 | 3.482 | 76.557 | 1.00 | 0.00 | xxxx | 1608 |
| ATOM | 1609 | CD2 | PHE | A | 206 | 22.889 | 3.258 | 74.969 | 1.00 | 0.00 | xxxx | 1609 |
| ATOM | 1610 | CE1 | PHE | A | 206 | 24.937 | 2.113 | 76.419 | 1.00 | 0.00 | xxxx | 1610 |
| ATOM | 1611 | CE2 | PHE | A | 206 | 23.158 | 1.899 | 74.835 | 1.00 | 0.00 | xxxx | 1611 |
| ATOM | 1612 | CZ | PHE | A | 206 | 24.180 | 1.336 | 75.555 | 1.00 | 0.00 | xxxx | 1612 |
| ATOM | 1613 | N | ALA | A | 207 | 22.503 | 8.018 | 78.005 | 1.00 | 0.00 | xxxx | 1613 |
| ATOM | 1614 | CA | ALA | A | 207 | 22.572 | 9.470 | 78.099 | 1.00 | 0.00 | xxxx | 1614 |
| ATOM | 1615 | C | ALA | A | 207 | 23.937 | 9.866 | 78.637 | 1.00 | 0.00 | xxxx | 1615 |
| ATOM | 1616 | O | ALA | A | 207 | 24.471 | 9.208 | 79.539 | 1.00 | 0.00 | xxxx | 1616 |
| ATOM | 1617 | CB | ALA | A | 207 | 21.467 | 10.027 | 79.005 | 1.00 | 0.00 | xxxx | 1617 |
| ATOM | 1618 | N | ASN | A | 208 | 24.498 | 10.944 | 78.082 | 1.00 | 0.00 | xxxx | 1618 |
| ATOM | 1619 | CA | ASN | A | 208 | 25.825 | 11.375 | 78.507 | 1.00 | 0.00 | xxxx | 1619 |
| ATOM | 1620 | C | ASN | A | 208 | 25.850 | 11.893 | 79.944 | 1.00 | 0.00 | xxxx | 1620 |
| ATOM | 1621 | O | ASN | A | 208 | 26.935 | 11.977 | 80.516 | 1.00 | 0.00 | xxxx | 1621 |
| ATOM | 1622 | CB | ASN | A | 208 | 26.378 | 12.499 | 77.614 | 1.00 | 0.00 | xxxx | 1622 |
| ATOM | 1623 | CG | ASN | A | 208 | 26.794 | 12.066 | 76.190 | 1.00 | 0.00 | xxxx | 1623 |
| ATOM | 1624 | OD1 | ASN | A | 208 | 27.122 | 12.946 | 75.398 | 1.00 | 0.00 | xxxx | 1624 |
| ATOM | 1625 | ND2 | ASN | A | 208 | 26.799 | 10.770 | 75.868 | 1.00 | 0.00 | xxxx | 1625 |
| ATOM | 1626 | N | ASN | A | 209 | 24.720 | 12.312 | 80.515 | 1.00 | 0.00 | xxxx | 1626 |
| ATOM | 1627 | CA | ASN | A | 209 | 24.716 | 12.706 | 81.922 | 1.00 | 0.00 | xxxx | 1627 |
| ATOM | 1628 | C | ASN | A | 209 | 23.396 | 12.327 | 82.587 | 1.00 | 0.00 | xxxx | 1628 |
| ATOM | 1629 | O | ASN | A | 209 | 22.425 | 11.927 | 81.931 | 1.00 | 0.00 | xxxx | 1629 |
| ATOM | 1630 | CB | ASN | A | 209 | 25.102 | 14.184 | 82.128 | 1.00 | 0.00 | xxxx | 1630 |
| ATOM | 1631 | CG | ASN | A | 209 | 23.948 | 15.150 | 81.859 | 1.00 | 0.00 | xxxx | 1631 |
| ATOM | 1632 | OD1 | ASN | A | 209 | 22.944 | 14.798 | 81.229 | 1.00 | 0.00 | xxxx | 1632 |
| ATOM | 1633 | ND2 | ASN | A | 209 | 24.091 | 16.374 | 82.348 | 1.00 | 0.00 | xxxx | 1633 |
| ATOM | 1634 | N | ASP | A | 210 | 23.383 | 12.465 | 83.917 | 1.00 | 0.00 | xxxx | 1634 |
| ATOM | 1635 | CA | ASP | A | 210 | 22.205 | 12.098 | 84.697 | 1.00 | 0.00 | xxxx | 1635 |
| ATOM | 1636 | C | ASP | A | 210 | 21.016 | 12.992 | 84.382 | 1.00 | 0.00 | xxxx | 1636 |
| ATOM | 1637 | O | ASP | A | 210 | 19.892 | 12.496 | 84.280 | 1.00 | 0.00 | xxxx | 1637 |
| ATOM | 1638 | CB | ASP | A | 210 | 22.499 | 12.120 | 86.200 | 1.00 | 0.00 | xxxx | 1638 |
| ATOM | 1639 | CG | ASP | A | 210 | 23.228 | 10.885 | 86.679 | 1.00 | 0.00 | xxxx | 1639 |
| ATOM | 1640 | OD1 | ASP | A | 210 | 23.451 | 9.951 | 85.873 | 1.00 | 0.00 | xxxx | 1640 |
| ATOM | 1641 | OD2 | ASP | A | 210 | 23.550 | 10.846 | 87.885 | 1.00 | 0.00 | xxxx | 1641 |
| ATOM | 1642 | N | ASP | A | 211 | 21.228 | 14.304 | 84.218 | 1.00 | 0.00 | xxxx | 1642 |
| ATOM | 1643 | CA | ASP | A | 211 | 20.071 | 15.166 | 83.960 | 1.00 | 0.00 | xxxx | 1643 |
| ATOM | 1644 | C | ASP | A | 211 | 19.346 | 14.769 | 82.678 | 1.00 | 0.00 | xxxx | 1644 |
| ATOM | 1645 | O | ASP | A | 211 | 18.109 | 14.733 | 82.643 | 1.00 | 0.00 | xxxx | 1645 |
| ATOM | 1646 | CB | ASP | A | 211 | 20.456 | 16.641 | 83.956 | 1.00 | 0.00 | xxxx | 1646 |
| ATOM | 1647 | CG | ASP | A | 211 | 20.279 | 17.293 | 85.323 | 1.00 | 0.00 | xxxx | 1647 |
| ATOM | 1648 | OD1 | ASP | A | 211 | 19.754 | 16.629 | 86.253 | 1.00 | 0.00 | xxxx | 1648 |
| ATOM | 1649 | OD2 | ASP | A | 211 | 20.643 | 18.479 | 85.456 | 1.00 | 0.00 | xxxx | 1649 |
| ATOM | 1650 | N | MET | A | 212 | 20.089 | 14.423 | 81.627 | 1.00 | 0.00 | xxxx | 1650 |
| ATOM | 1651 | CA | MET | A | 212 | 19.418 | 13.970 | 80.413 | 1.00 | 0.00 | xxxx | 1651 |
| ATOM | 1652 | C | MET | A | 212 | 18.766 | 12.608 | 80.614 | 1.00 | 0.00 | xxxx | 1652 |
| ATOM | 1653 | O | MET | A | 212 | 17.664 | 12.359 | 80.105 | 1.00 | 0.00 | xxxx | 1653 |
| ATOM | 1654 | CB | MET | A | 212 | 20.388 | 13.937 | 79.232 | 1.00 | 0.00 | xxxx | 1654 |
| ATOM | 1655 | CG | MET | A | 212 | 20.811 | 15.334 | 78.819 | 1.00 | 0.00 | xxxx | 1655 |
| ATOM | 1656 | SD | MET | A | 212 | 21.785 | 15.350 | 77.315 | 1.00 | 0.00 | xxxx | 1656 |
| ATOM | 1657 | CE | MET | A | 212 | 23.183 | 14.347 | 77.803 | 1.00 | 0.00 | xxxx | 1657 |
| ATOM | 1658 | N | ALA | A | 213 | 19.418 | 11.707 | 81.356 | 1.00 | 0.00 | xxxx | 1658 |
| ATOM | 1659 | CA | ALA | A | 213 | 18.786 | 10.419 | 81.642 | 1.00 | 0.00 | xxxx | 1659 |
| ATOM | 1660 | C | ALA | A | 213 | 17.469 | 10.612 | 82.378 | 1.00 | 0.00 | xxxx | 1660 |
| ATOM | 1661 | O | ALA | A | 213 | 16.484 | 9.916 | 82.096 | 1.00 | 0.00 | xxxx | 1661 |
| ATOM | 1662 | CB | ALA | A | 213 | 19.728 | 9.536 | 82.462 | 1.00 | 0.00 | xxxx | 1662 |
| ATOM | 1663 | N | LEU | A | 214 | 17.433 | 11.567 | 83.311 | 1.00 | 0.00 | xxxx | 1663 |
| ATOM | 1664 | CA | LEU | A | 214 | 16.230 | 11.827 | 84.099 | 1.00 | 0.00 | xxxx | 1664 |
| ATOM | 1665 | C | LEU | A | 214 | 15.123 | 12.421 | 83.242 | 1.00 | 0.00 | xxxx | 1665 |
| ATOM | 1666 | O | LEU | A | 214 | 13.948 | 12.090 | 83.429 | 1.00 | 0.00 | xxxx | 1666 |
| ATOM | 1667 | CB | LEU | A | 214 | 16.562 | 12.751 | 85.271 | 1.00 | 0.00 | xxxx | 1667 |
| ATOM | 1668 | CG | LEU | A | 214 | 17.503 | 12.139 | 86.310 | 1.00 | 0.00 | xxxx | 1668 |
| ATOM | 1669 | CD1 | LEU | A | 214 | 18.009 | 13.201 | 87.272 | 1.00 | 0.00 | xxxx | 1669 |
| ATOM | 1670 | CD2 | LEU | A | 214 | 16.789 | 11.031 | 87.065 | 1.00 | 0.00 | xxxx | 1670 |
| ATOM | 1671 | N | GLY | A | 215 | 15.471 | 13.308 | 82.309 | 1.00 | 0.00 | xxxx | 1671 |
| ATOM | 1672 | CA | GLY | A | 215 | 14.475 | 13.776 | 81.359 | 1.00 | 0.00 | xxxx | 1672 |
| ATOM | 1673 | C | GLY | A | 215 | 13.929 | 12.650 | 80.503 | 1.00 | 0.00 | xxxx | 1673 |
| ATOM | 1674 | O | GLY | A | 215 | 12.723 | 12.584 | 80.244 | 1.00 | 0.00 | xxxx | 1674 |
| ATOM | 1675 | N | ALA | A | 216 | 14.806 | 11.757 | 80.039 | 1.00 | 0.00 | xxxx | 1675 |
| ATOM | 1676 | CA | ALA | A | 216 | 14.338 | 10.602 | 79.279 | 1.00 | 0.00 | xxxx | 1676 |
| ATOM | 1677 | C | ALA | A | 216 | 13.396 | 9.751 | 80.110 | 1.00 | 0.00 | xxxx | 1677 |
| ATOM | 1678 | O | ALA | A | 216 | 12.360 | 9.292 | 79.612 | 1.00 | 0.00 | xxxx | 1678 |
| ATOM | 1679 | CB | ALA | A | 216 | 15.529 | 9.769 | 78.802 | 1.00 | 0.00 | xxxx | 1679 |
| ATOM | 1680 | N | ILE | A | 217 | 13.738 | 9.538 | 81.385 | 1.00 | 0.00 | xxxx | 1680 |
| ATOM | 1681 | CA | ILE | A | 217 | 12.897 | 8.739 | 82.277 | 1.00 | 0.00 | xxxx | 1681 |
| ATOM | 1682 | C | ILE | A | 217 | 11.510 | 9.355 | 82.420 | 1.00 | 0.00 | xxxx | 1682 |
| ATOM | 1683 | O | ILE | A | 217 | 10.499 | 8.646 | 82.418 | 1.00 | 0.00 | xxxx | 1683 |
| ATOM | 1684 | CB | ILE | A | 217 | 13.605 | 8.537 | 83.627 | 1.00 | 0.00 | xxxx | 1684 |
| ATOM | 1685 | CG1 | ILE | A | 217 | 14.716 | 7.494 | 83.461 | 1.00 | 0.00 | xxxx | 1685 |
| ATOM | 1686 | CG2 | ILE | A | 217 | 12.598 | 8.125 | 84.691 | 1.00 | 0.00 | xxxx | 1686 |
| ATOM | 1687 | CD1 | ILE | A | 217 | 15.733 | 7.486 | 84.585 | 1.00 | 0.00 | xxxx | 1687 |
| ATOM | 1688 | N | GLU | A | 218 | 11.430 | 10.685 | 82.521 | 1.00 | 0.00 | xxxx | 1688 |
| ATOM | 1689 | CA | GLU | A | 218 | 10.119 | 11.329 | 82.600 | 1.00 | 0.00 | xxxx | 1689 |
| ATOM | 1690 | C | GLU | A | 218 | 9.289 | 11.080 | 81.342 | 1.00 | 0.00 | xxxx | 1690 |
| ATOM | 1691 | O | GLU | A | 218 | 8.080 | 10.843 | 81.427 | 1.00 | 0.00 | xxxx | 1691 |
| ATOM | 1692 | CB | GLU | A | 218 | 10.273 | 12.826 | 82.880 | 1.00 | 0.00 | xxxx | 1692 |
| ATOM | 1693 | CG | GLU | A | 218 | 10.768 | 13.106 | 84.295 | 1.00 | 0.00 | xxxx | 1693 |
| ATOM | 1694 | CD | GLU | A | 218 | 9.954 | 12.360 | 85.351 | 1.00 | 0.00 | xxxx | 1694 |
| ATOM | 1695 | OE1 | GLU | A | 218 | 8.711 | 12.546 | 85.409 | 1.00 | 0.00 | xxxx | 1695 |
| ATOM | 1696 | OE2 | GLU | A | 218 | 10.550 | 11.574 | 86.125 | 1.00 | 0.00 | xxxx | 1696 |
| ATOM | 1697 | N | ALA | A | 219 | 9.917 | 11.127 | 80.162 | 1.00 | 0.00 | xxxx | 1697 |
| ATOM | 1698 | CA | ALA | A | 219 | 9.177 | 10.831 | 78.937 | 1.00 | 0.00 | xxxx | 1698 |
| ATOM | 1699 | C | ALA | A | 219 | 8.754 | 9.368 | 78.887 | 1.00 | 0.00 | xxxx | 1699 |
| ATOM | 1700 | O | ALA | A | 219 | 7.645 | 9.048 | 78.448 | 1.00 | 0.00 | xxxx | 1700 |
| ATOM | 1701 | CB | ALA | A | 219 | 10.025 | 11.184 | 77.715 | 1.00 | 0.00 | xxxx | 1701 |
| ATOM | 1702 | N | LEU | A | 220 | 9.623 | 8.468 | 79.358 | 1.00 | 0.00 | xxxx | 1702 |
| ATOM | 1703 | CA | LEU | A | 220 | 9.266 | 7.051 | 79.431 | 1.00 | 0.00 | xxxx | 1703 |
| ATOM | 1704 | C | LEU | A | 220 | 8.105 | 6.809 | 80.392 | 1.00 | 0.00 | xxxx | 1704 |
| ATOM | 1705 | O | LEU | A | 220 | 7.181 | 6.049 | 80.078 | 1.00 | 0.00 | xxxx | 1705 |
| ATOM | 1706 | CB | LEU | A | 220 | 10.492 | 6.229 | 79.828 | 1.00 | 0.00 | xxxx | 1706 |
| ATOM | 1707 | CG | LEU | A | 220 | 11.577 | 6.146 | 78.748 | 1.00 | 0.00 | xxxx | 1707 |
| ATOM | 1708 | CD1 | LEU | A | 220 | 12.935 | 5.811 | 79.350 | 1.00 | 0.00 | xxxx | 1708 |
| ATOM | 1709 | CD2 | LEU | A | 220 | 11.184 | 5.099 | 77.725 | 1.00 | 0.00 | xxxx | 1709 |
| ATOM | 1710 | N | LYS | A | 221 | 8.129 | 7.452 | 81.566 | 1.00 | 0.00 | xxxx | 1710 |
| ATOM | 1711 | CA | LYS | A | 221 | 7.009 | 7.320 | 82.497 | 1.00 | 0.00 | xxxx | 1711 |
| ATOM | 1712 | C | LYS | A | 221 | 5.709 | 7.782 | 81.858 | 1.00 | 0.00 | xxxx | 1712 |
| ATOM | 1713 | O | LYS | A | 221 | 4.675 | 7.119 | 81.991 | 1.00 | 0.00 | xxxx | 1713 |
| ATOM | 1714 | CB | LYS | A | 221 | 7.284 | 8.142 | 83.756 | 1.00 | 0.00 | xxxx | 1714 |
| ATOM | 1715 | CG | LYS | A | 221 | 8.319 | 7.544 | 84.684 | 1.00 | 0.00 | xxxx | 1715 |
| ATOM | 1716 | CD | LYS | A | 221 | 8.604 | 8.504 | 85.825 | 1.00 | 0.00 | xxxx | 1716 |
| ATOM | 1717 | CE | LYS | A | 221 | 9.505 | 7.891 | 86.871 | 1.00 | 0.00 | xxxx | 1717 |
| ATOM | 1718 | NZ | LYS | A | 221 | 9.758 | 8.870 | 87.968 | 1.00 | 0.00 | xxxx | 1718 |
| ATOM | 1719 | N | SER | A | 222 | 5.750 | 8.912 | 81.144 | 1.00 | 0.00 | xxxx | 1719 |
| ATOM | 1720 | CA | SER | A | 222 | 4.546 | 9.442 | 80.517 | 1.00 | 0.00 | xxxx | 1720 |
| ATOM | 1721 | C | SER | A | 222 | 3.975 | 8.470 | 79.493 | 1.00 | 0.00 | xxxx | 1721 |
| ATOM | 1722 | O | SER | A | 222 | 2.756 | 8.427 | 79.292 | 1.00 | 0.00 | xxxx | 1722 |
| ATOM | 1723 | CB | SER | A | 222 | 4.887 | 10.783 | 79.868 | 1.00 | 0.00 | xxxx | 1723 |
| ATOM | 1724 | OG | SER | A | 222 | 3.742 | 11.388 | 79.291 | 1.00 | 0.00 | xxxx | 1724 |
| ATOM | 1725 | N | ALA | A | 223 | 4.835 | 7.670 | 78.859 | 1.00 | 0.00 | xxxx | 1725 |
| ATOM | 1726 | CA | ALA | A | 223 | 4.440 | 6.652 | 77.894 | 1.00 | 0.00 | xxxx | 1726 |
| ATOM | 1727 | C | ALA | A | 223 | 4.151 | 5.298 | 78.533 | 1.00 | 0.00 | xxxx | 1727 |
| ATOM | 1728 | O | ALA | A | 223 | 3.934 | 4.323 | 77.806 | 1.00 | 0.00 | xxxx | 1728 |
| ATOM | 1729 | CB | ALA | A | 223 | 5.525 | 6.498 | 76.820 | 1.00 | 0.00 | xxxx | 1729 |
| ATOM | 1730 | N | GLY | A | 224 | 4.168 | 5.203 | 79.857 | 1.00 | 0.00 | xxxx | 1730 |
| ATOM | 1731 | CA | GLY | A | 224 | 3.714 | 4.007 | 80.540 | 1.00 | 0.00 | xxxx | 1731 |
| ATOM | 1732 | C | GLY | A | 224 | 4.788 | 3.065 | 81.044 | 1.00 | 0.00 | xxxx | 1732 |
| ATOM | 1733 | O | GLY | A | 224 | 4.453 | 1.960 | 81.485 | 1.00 | 0.00 | xxxx | 1733 |
| ATOM | 1734 | N | TYR | A | 225 | 6.059 | 3.453 | 80.996 | 1.00 | 0.00 | xxxx | 1734 |
| ATOM | 1735 | CA | TYR | A | 225 | 7.105 | 2.588 | 81.524 | 1.00 | 0.00 | xxxx | 1735 |
| ATOM | 1736 | C | TYR | A | 225 | 7.240 | 2.767 | 83.030 | 1.00 | 0.00 | xxxx | 1736 |
| ATOM | 1737 | O | TYR | A | 225 | 6.821 | 3.775 | 83.605 | 1.00 | 0.00 | xxxx | 1737 |
| ATOM | 1738 | CB | TYR | A | 225 | 8.454 | 2.918 | 80.882 | 1.00 | 0.00 | xxxx | 1738 |
| ATOM | 1739 | CG | TYR | A | 225 | 8.570 | 2.480 | 79.443 | 1.00 | 0.00 | xxxx | 1739 |
| ATOM | 1740 | CD1 | TYR | A | 225 | 8.000 | 3.229 | 78.424 | 1.00 | 0.00 | xxxx | 1740 |
| ATOM | 1741 | CD2 | TYR | A | 225 | 9.239 | 1.309 | 79.106 | 1.00 | 0.00 | xxxx | 1741 |
| ATOM | 1742 | CE1 | TYR | A | 225 | 8.099 | 2.830 | 77.102 | 1.00 | 0.00 | xxxx | 1742 |
| ATOM | 1743 | CE2 | TYR | A | 225 | 9.345 | 0.901 | 77.786 | 1.00 | 0.00 | xxxx | 1743 |
| ATOM | 1744 | CZ | TYR | A | 225 | 8.774 | 1.667 | 76.790 | 1.00 | 0.00 | xxxx | 1744 |
| ATOM | 1745 | OH | TYR | A | 225 | 8.868 | 1.275 | 75.474 | 1.00 | 0.00 | xxxx | 1745 |
| ATOM | 1746 | N | PHE | A | 226 | 7.838 | 1.757 | 83.665 | 1.00 | 0.00 | xxxx | 1746 |
| ATOM | 1747 | CA | PHE | A | 226 | 8.172 | 1.755 | 85.089 | 1.00 | 0.00 | xxxx | 1747 |
| ATOM | 1748 | C | PHE | A | 226 | 6.955 | 1.612 | 85.993 | 1.00 | 0.00 | xxxx | 1748 |
| ATOM | 1749 | O | PHE | A | 226 | 7.021 | 1.935 | 87.183 | 1.00 | 0.00 | xxxx | 1749 |
| ATOM | 1750 | CB | PHE | A | 226 | 9.049 | 2.945 | 85.479 | 1.00 | 0.00 | xxxx | 1750 |
| ATOM | 1751 | CG | PHE | A | 226 | 10.263 | 3.081 | 84.613 | 1.00 | 0.00 | xxxx | 1751 |
| ATOM | 1752 | CD1 | PHE | A | 226 | 11.193 | 2.054 | 84.551 | 1.00 | 0.00 | xxxx | 1752 |
| ATOM | 1753 | CD2 | PHE | A | 226 | 10.472 | 4.221 | 83.846 | 1.00 | 0.00 | xxxx | 1753 |
| ATOM | 1754 | CE1 | PHE | A | 226 | 12.318 | 2.161 | 83.746 | 1.00 | 0.00 | xxxx | 1754 |
| ATOM | 1755 | CE2 | PHE | A | 226 | 11.592 | 4.336 | 83.036 | 1.00 | 0.00 | xxxx | 1755 |
| ATOM | 1756 | CZ | PHE | A | 226 | 12.521 | 3.300 | 82.988 | 1.00 | 0.00 | xxxx | 1756 |
| ATOM | 1757 | N | THR | A | 227 | 5.843 | 1.150 | 85.430 | 1.00 | 0.00 | xxxx | 1757 |
| ATOM | 1758 | CA | THR | A | 227 | 4.682 | 0.684 | 86.169 | 1.00 | 0.00 | xxxx | 1758 |
| ATOM | 1759 | C | THR | A | 227 | 4.250 | −0.624 | 85.528 | 1.00 | 0.00 | xxxx | 1759 |
| ATOM | 1760 | O | THR | A | 227 | 4.433 | −0.828 | 84.324 | 1.00 | 0.00 | xxxx | 1760 |
| ATOM | 1761 | CB | THR | A | 227 | 3.523 | 1.686 | 86.088 | 1.00 | 0.00 | xxxx | 1761 |
| ATOM | 1762 | OG1 | THR | A | 227 | 3.120 | 1.844 | 84.721 | 1.00 | 0.00 | xxxx | 1762 |
| ATOM | 1763 | CG2 | THR | A | 227 | 3.933 | 3.041 | 86.656 | 1.00 | 0.00 | xxxx | 1763 |
| ATOM | 1764 | N | GLY | A | 228 | 3.678 | −1.510 | 86.331 | 1.00 | 0.00 | xxxx | 1764 |
| ATOM | 1765 | CA | GLY | A | 228 | 3.389 | −2.839 | 85.817 | 1.00 | 0.00 | xxxx | 1765 |
| ATOM | 1766 | C | GLY | A | 228 | 4.687 | −3.566 | 85.519 | 1.00 | 0.00 | xxxx | 1766 |
| ATOM | 1767 | O | GLY | A | 228 | 5.616 | −3.577 | 86.333 | 1.00 | 0.00 | xxxx | 1767 |
| ATOM | 1768 | N | ASN | A | 229 | 4.770 | −4.170 | 84.336 | 1.00 | 0.00 | xxxx | 1768 |
| ATOM | 1769 | CA | ASN | A | 229 | 5.949 | −4.930 | 83.939 | 1.00 | 0.00 | xxxx | 1769 |
| ATOM | 1770 | C | ASN | A | 229 | 6.702 | −4.298 | 82.773 | 1.00 | 0.00 | xxxx | 1770 |
| ATOM | 1771 | O | ASN | A | 229 | 7.568 | −4.953 | 82.181 | 1.00 | 0.00 | xxxx | 1771 |
| ATOM | 1772 | CB | ASN | A | 229 | 5.568 | −6.377 | 83.610 | 1.00 | 0.00 | xxxx | 1772 |
| ATOM | 1773 | CG | ASN | A | 229 | 4.838 | −7.063 | 84.752 | 1.00 | 0.00 | xxxx | 1773 |
| ATOM | 1774 | OD1 | ASN | A | 229 | 3.804 | −7.703 | 84.550 | 1.00 | 0.00 | xxxx | 1774 |
| ATOM | 1775 | ND2 | ASN | A | 229 | 5.372 | −6.928 | 85.962 | 1.00 | 0.00 | xxxx | 1775 |
| ATOM | 1776 | N | LYS | A | 230 | 6.405 | −3.042 | 82.438 | 1.00 | 0.00 | xxxx | 1776 |
| ATOM | 1777 | CA | LYS | A | 230 | 6.967 | −2.385 | 81.260 | 1.00 | 0.00 | xxxx | 1777 |
| ATOM | 1778 | C | LYS | A | 230 | 8.247 | −1.659 | 81.660 | 1.00 | 0.00 | xxxx | 1778 |
| ATOM | 1779 | O | LYS | A | 230 | 8.196 | −0.602 | 82.292 | 1.00 | 0.00 | xxxx | 1779 |
| ATOM | 1780 | CB | LYS | A | 230 | 5.952 | −1.404 | 80.685 | 1.00 | 0.00 | xxxx | 1780 |
| ATOM | 1781 | CG | LYS | A | 230 | 6.277 | −0.908 | 79.292 | 1.00 | 0.00 | xxxx | 1781 |
| ATOM | 1782 | CD | LYS | A | 230 | 5.185 | 0.026 | 78.796 | 1.00 | 0.00 | xxxx | 1782 |
| ATOM | 1783 | CE | LYS | A | 230 | 5.326 | 0.329 | 77.315 | 1.00 | 0.00 | xxxx | 1783 |
| ATOM | 1784 | NZ | LYS | A | 230 | 4.308 | 1.323 | 76.869 | 1.00 | 0.00 | xxxx | 1784 |
| ATOM | 1785 | N | TYR | A | 231 | 9.391 | −2.211 | 81.264 | 1.00 | 0.00 | xxxx | 1785 |
| ATOM | 1786 | CA | TYR | A | 231 | 10.690 | −1.754 | 81.735 | 1.00 | 0.00 | xxxx | 1786 |
| ATOM | 1787 | C | TYR | A | 231 | 11.625 | −1.492 | 80.561 | 1.00 | 0.00 | xxxx | 1787 |
| ATOM | 1788 | O | TYR | A | 231 | 11.531 | −2.142 | 79.519 | 1.00 | 0.00 | xxxx | 1788 |
| ATOM | 1789 | CB | TYR | A | 231 | 11.305 | −2.813 | 82.651 | 1.00 | 0.00 | xxxx | 1789 |
| ATOM | 1790 | CG | TYR | A | 231 | 12.673 | −2.447 | 83.152 | 1.00 | 0.00 | xxxx | 1790 |
| ATOM | 1791 | CD1 | TYR | A | 231 | 12.824 | −1.635 | 84.264 | 1.00 | 0.00 | xxxx | 1791 |
| ATOM | 1792 | CD2 | TYR | A | 231 | 13.814 | −2.906 | 82.508 | 1.00 | 0.00 | xxxx | 1792 |
| ATOM | 1793 | CE1 | TYR | A | 231 | 14.077 | −1.293 | 84.726 | 1.00 | 0.00 | xxxx | 1793 |
| ATOM | 1794 | CE2 | TYR | A | 231 | 15.065 | −2.574 | 82.960 | 1.00 | 0.00 | xxxx | 1794 |
| ATOM | 1795 | CZ | TYR | A | 231 | 15.197 | −1.764 | 84.060 | 1.00 | 0.00 | xxxx | 1795 |
| ATOM | 1796 | OH | TYR | A | 231 | 16.452 | −1.437 | 84.506 | 1.00 | 0.00 | xxxx | 1796 |
| ATOM | 1797 | N | ILE | A | 232 | 12.542 | −0.539 | 80.740 | 1.00 | 0.00 | xxxx | 1797 |
| ATOM | 1798 | CA | ILE | A | 232 | 13.594 | −0.267 | 79.760 | 1.00 | 0.00 | xxxx | 1798 |
| ATOM | 1799 | C | ILE | A | 232 | 14.838 | 0.172 | 80.524 | 1.00 | 0.00 | xxxx | 1799 |
| ATOM | 1800 | O | ILE | A | 232 | 14.719 | 0.936 | 81.494 | 1.00 | 0.00 | xxxx | 1800 |
| ATOM | 1801 | CB | ILE | A | 232 | 13.147 | 0.780 | 78.728 | 1.00 | 0.00 | xxxx | 1801 |
| ATOM | 1802 | CG1 | ILE | A | 232 | 14.186 | 0.924 | 77.622 | 1.00 | 0.00 | xxxx | 1802 |
| ATOM | 1803 | CG2 | ILE | A | 232 | 12.864 | 2.122 | 79.397 | 1.00 | 0.00 | xxxx | 1803 |
| ATOM | 1804 | CD1 | ILE | A | 232 | 13.684 | 1.726 | 76.428 | 1.00 | 0.00 | xxxx | 1804 |
| ATOM | 1805 | N | PRO | A | 233 | 16.035 | −0.298 | 80.160 | 1.00 | 0.00 | xxxx | 1805 |
| ATOM | 1806 | CA | PRO | A | 233 | 17.246 | 0.122 | 80.880 | 1.00 | 0.00 | xxxx | 1806 |
| ATOM | 1807 | C | PRO | A | 233 | 17.726 | 1.475 | 80.372 | 1.00 | 0.00 | xxxx | 1807 |
| ATOM | 1808 | O | PRO | A | 233 | 17.838 | 1.700 | 79.163 | 1.00 | 0.00 | xxxx | 1808 |
| ATOM | 1809 | CB | PRO | A | 233 | 18.260 | −0.975 | 80.535 | 1.00 | 0.00 | xxxx | 1809 |
| ATOM | 1810 | CG | PRO | A | 233 | 17.816 | −1.455 | 79.173 | 1.00 | 0.00 | xxxx | 1810 |
| ATOM | 1811 | CD | PRO | A | 233 | 16.306 | −1.417 | 79.237 | 1.00 | 0.00 | xxxx | 1811 |
| ATOM | 1812 | N | VAL | A | 234 | 18.000 | 2.378 | 81.311 | 1.00 | 0.00 | xxxx | 1812 |
| ATOM | 1813 | CA | VAL | A | 234 | 18.495 | 3.726 | 81.046 | 1.00 | 0.00 | xxxx | 1813 |
| ATOM | 1814 | C | VAL | A | 234 | 19.765 | 3.893 | 81.869 | 1.00 | 0.00 | xxxx | 1814 |
| ATOM | 1815 | O | VAL | A | 234 | 19.782 | 3.535 | 83.053 | 1.00 | 0.00 | xxxx | 1815 |
| ATOM | 1816 | CB | VAL | A | 234 | 17.460 | 4.778 | 81.487 | 1.00 | 0.00 | xxxx | 1816 |
| ATOM | 1817 | CG1 | VAL | A | 234 | 17.978 | 6.191 | 81.195 | 1.00 | 0.00 | xxxx | 1817 |
| ATOM | 1818 | CG2 | VAL | A | 234 | 16.112 | 4.530 | 80.804 | 1.00 | 0.00 | xxxx | 1818 |
| ATOM | 1819 | N | VAL | A | 235 | 20.823 | 4.394 | 81.240 | 1.00 | 0.00 | xxxx | 1819 |
| ATOM | 1820 | CA | VAL | A | 235 | 22.057 | 4.675 | 81.959 | 1.00 | 0.00 | xxxx | 1820 |
| ATOM | 1821 | C | VAL | A | 235 | 22.430 | 6.139 | 81.791 | 1.00 | 0.00 | xxxx | 1821 |
| ATOM | 1822 | O | VAL | A | 235 | 22.133 | 6.778 | 80.774 | 1.00 | 0.00 | xxxx | 1822 |
| ATOM | 1823 | CB | VAL | A | 235 | 23.217 | 3.756 | 81.523 | 1.00 | 0.00 | xxxx | 1823 |
| ATOM | 1824 | CG1 | VAL | A | 235 | 22.953 | 2.340 | 81.988 | 1.00 | 0.00 | xxxx | 1824 |
| ATOM | 1825 | CG2 | VAL | A | 235 | 23.378 | 3.811 | 80.029 | 1.00 | 0.00 | xxxx | 1825 |
| ATOM | 1826 | N | GLY | A | 236 | 23.088 | 6.666 | 82.819 | 1.00 | 0.00 | xxxx | 1826 |
| ATOM | 1827 | CA | GLY | A | 236 | 23.609 | 8.026 | 82.778 | 1.00 | 0.00 | xxxx | 1827 |
| ATOM | 1828 | C | GLY | A | 236 | 25.040 | 8.151 | 83.282 | 1.00 | 0.00 | xxxx | 1828 |
| ATOM | 1829 | O | GLY | A | 236 | 25.756 | 7.147 | 83.372 | 1.00 | 0.00 | xxxx | 1829 |
| ATOM | 1830 | N | VAL | A | 237 | 25.485 | 9.375 | 83.586 | 1.00 | 0.00 | xxxx | 1830 |
| ATOM | 1831 | CA | VAL | A | 237 | 26.766 | 9.634 | 84.252 | 1.00 | 0.00 | xxxx | 1831 |
| ATOM | 1832 | C | VAL | A | 237 | 26.558 | 10.798 | 85.209 | 1.00 | 0.00 | xxxx | 1832 |
| ATOM | 1833 | O | VAL | A | 237 | 25.967 | 11.808 | 84.815 | 1.00 | 0.00 | xxxx | 1833 |
| ATOM | 1834 | CB | VAL | A | 237 | 27.894 | 10.024 | 83.266 | 1.00 | 0.00 | xxxx | 1834 |
| ATOM | 1835 | CG1 | VAL | A | 237 | 29.137 | 10.427 | 84.038 | 1.00 | 0.00 | xxxx | 1835 |
| ATOM | 1836 | CG2 | VAL | A | 237 | 28.220 | 8.897 | 82.312 | 1.00 | 0.00 | xxxx | 1836 |
| ATOM | 1837 | N | ASP | A | 238 | 27.024 | 10.642 | 86.462 | 1.00 | 0.00 | xxxx | 1837 |
| ATOM | 1838 | CA | ASP | A | 238 | 27.345 | 11.681 | 87.450 | 1.00 | 0.00 | xxxx | 1838 |
| ATOM | 1839 | C | ASP | A | 238 | 27.114 | 11.178 | 88.868 | 1.00 | 0.00 | xxxx | 1839 |
| ATOM | 1840 | O | ASP | A | 238 | 27.899 | 11.481 | 89.767 | 1.00 | 0.00 | xxxx | 1840 |
| ATOM | 1841 | CB | ASP | A | 238 | 26.536 | 12.973 | 87.299 | 1.00 | 0.00 | xxxx | 1841 |
| ATOM | 1842 | CG | ASP | A | 238 | 27.067 | 13.885 | 86.218 | 1.00 | 0.00 | xxxx | 1842 |
| ATOM | 1843 | OD1 | ASP | A | 238 | 28.247 | 13.776 | 85.824 | 1.00 | 0.00 | xxxx | 1843 |
| ATOM | 1844 | OD2 | ASP | A | 238 | 26.279 | 14.734 | 85.762 | 1.00 | 0.00 | xxxx | 1844 |
| ATOM | 1845 | N | ALA | A | 239 | 26.025 | 10.432 | 89.073 | 1.00 | 0.00 | xxxx | 1845 |
| ATOM | 1846 | CA | ALA | A | 239 | 25.549 | 10.070 | 90.408 | 1.00 | 0.00 | xxxx | 1846 |
| ATOM | 1847 | C | ALA | A | 239 | 25.271 | 11.317 | 91.243 | 1.00 | 0.00 | xxxx | 1847 |
| ATOM | 1848 | O | ALA | A | 239 | 25.662 | 11.417 | 92.406 | 1.00 | 0.00 | xxxx | 1848 |
| ATOM | 1849 | CB | ALA | A | 239 | 26.493 | 9.092 | 91.121 | 1.00 | 0.00 | xxxx | 1849 |
| ATOM | 1850 | N | THR | A | 240 | 24.590 | 12.283 | 90.626 | 1.00 | 0.00 | xxxx | 1850 |
| ATOM | 1851 | CA | THR | A | 240 | 24.058 | 13.399 | 91.396 | 1.00 | 0.00 | xxxx | 1851 |
| ATOM | 1852 | C | THR | A | 240 | 22.968 | 12.895 | 92.340 | 1.00 | 0.00 | xxxx | 1852 |
| ATOM | 1853 | O | THR | A | 240 | 22.496 | 11.758 | 92.239 | 1.00 | 0.00 | xxxx | 1853 |
| ATOM | 1854 | CB | THR | A | 240 | 23.417 | 14.440 | 90.476 | 1.00 | 0.00 | xxxx | 1854 |
| ATOM | 1855 | OG1 | THR | A | 240 | 22.331 | 13.837 | 89.764 | 1.00 | 0.00 | xxxx | 1855 |
| ATOM | 1856 | CG2 | THR | A | 240 | 24.422 | 15.016 | 89.473 | 1.00 | 0.00 | xxxx | 1856 |
| ATOM | 1857 | N | ALA | A | 241 | 22.542 | 13.762 | 93.258 | 1.00 | 0.00 | xxxx | 1857 |
| ATOM | 1858 | CA | ALA | A | 241 | 21.444 | 13.384 | 94.147 | 1.00 | 0.00 | xxxx | 1858 |
| ATOM | 1859 | C | ALA | A | 241 | 20.197 | 12.922 | 93.397 | 1.00 | 0.00 | xxxx | 1859 |
| ATOM | 1860 | O | ALA | A | 241 | 19.662 | 11.858 | 93.744 | 1.00 | 0.00 | xxxx | 1860 |
| ATOM | 1861 | CB | ALA | A | 241 | 21.143 | 14.492 | 95.165 | 1.00 | 0.00 | xxxx | 1861 |
| ATOM | 1862 | N | PRO | A | 242 | 19.696 | 13.626 | 92.375 | 1.00 | 0.00 | xxxx | 1862 |
| ATOM | 1863 | CA | PRO | A | 242 | 18.529 | 13.084 | 91.664 | 1.00 | 0.00 | xxxx | 1863 |
| ATOM | 1864 | C | PRO | A | 242 | 18.840 | 11.821 | 90.880 | 1.00 | 0.00 | xxxx | 1864 |
| ATOM | 1865 | O | PRO | A | 242 | 17.951 | 10.979 | 90.725 | 1.00 | 0.00 | xxxx | 1865 |
| ATOM | 1866 | CB | PRO | A | 242 | 18.056 | 14.241 | 90.771 | 1.00 | 0.00 | xxxx | 1866 |
| ATOM | 1867 | CG | PRO | A | 242 | 19.187 | 15.185 | 90.708 | 1.00 | 0.00 | xxxx | 1867 |
| ATOM | 1868 | CD | PRO | A | 242 | 20.012 | 15.003 | 91.949 | 1.00 | 0.00 | xxxx | 1868 |
| ATOM | 1869 | N | GLY | A | 243 | 20.072 | 11.652 | 90.396 | 1.00 | 0.00 | xxxx | 1869 |
| ATOM | 1870 | CA | GLY | A | 243 | 20.421 | 10.405 | 89.729 | 1.00 | 0.00 | xxxx | 1870 |
| ATOM | 1871 | C | GLY | A | 243 | 20.411 | 9.234 | 90.694 | 1.00 | 0.00 | xxxx | 1871 |
| ATOM | 1872 | O | GLY | A | 243 | 19.869 | 8.164 | 90.392 | 1.00 | 0.00 | xxxx | 1872 |
| ATOM | 1873 | N | ILE | A | 244 | 20.995 | 9.423 | 91.880 | 1.00 | 0.00 | xxxx | 1873 |
| ATOM | 1874 | CA | ILE | A | 244 | 20.952 | 8.389 | 92.913 | 1.00 | 0.00 | xxxx | 1874 |
| ATOM | 1875 | C | ILE | A | 244 | 19.513 | 8.030 | 93.257 | 1.00 | 0.00 | xxxx | 1875 |
| ATOM | 1876 | O | ILE | A | 244 | 19.168 | 6.850 | 93.408 | 1.00 | 0.00 | xxxx | 1876 |
| ATOM | 1877 | CB | ILE | A | 244 | 21.745 | 8.848 | 94.148 | 1.00 | 0.00 | xxxx | 1877 |
| ATOM | 1878 | CG1 | ILE | A | 244 | 23.233 | 8.926 | 93.802 | 1.00 | 0.00 | xxxx | 1878 |
| ATOM | 1879 | CG2 | ILE | A | 244 | 21.491 | 7.935 | 95.335 | 1.00 | 0.00 | xxxx | 1879 |
| ATOM | 1880 | CD1 | ILE | A | 244 | 24.048 | 9.638 | 94.852 | 1.00 | 0.00 | xxxx | 1880 |
| ATOM | 1881 | N | GLN | A | 245 | 18.644 | 9.036 | 93.363 | 1.00 | 0.00 | xxxx | 1881 |
| ATOM | 1882 | CA | GLN | A | 245 | 17.257 | 8.751 | 93.706 | 1.00 | 0.00 | xxxx | 1882 |
| ATOM | 1883 | C | GLN | A | 245 | 16.587 | 7.904 | 92.626 | 1.00 | 0.00 | xxxx | 1883 |
| ATOM | 1884 | O | GLN | A | 245 | 15.791 | 7.013 | 92.938 | 1.00 | 0.00 | xxxx | 1884 |
| ATOM | 1885 | CB | GLN | A | 245 | 16.496 | 10.053 | 93.937 | 1.00 | 0.00 | xxxx | 1885 |
| ATOM | 1886 | CG | GLN | A | 245 | 15.085 | 9.835 | 94.443 | 1.00 | 0.00 | xxxx | 1886 |
| ATOM | 1887 | CD | GLN | A | 245 | 15.060 | 9.092 | 95.773 | 1.00 | 0.00 | xxxx | 1887 |
| ATOM | 1888 | OE1 | GLN | A | 245 | 15.750 | 9.467 | 96.722 | 1.00 | 0.00 | xxxx | 1888 |
| ATOM | 1889 | NE2 | GLN | A | 245 | 14.295 | 8.014 | 95.832 | 1.00 | 0.00 | xxxx | 1889 |
| ATOM | 1890 | N | ALA | A | 246 | 16.919 | 8.145 | 91.353 | 1.00 | 0.00 | xxxx | 1890 |
| ATOM | 1891 | CA | ALA | A | 246 | 16.345 | 7.341 | 90.279 | 1.00 | 0.00 | xxxx | 1891 |
| ATOM | 1892 | C | ALA | A | 246 | 16.863 | 5.904 | 90.301 | 1.00 | 0.00 | xxxx | 1892 |
| ATOM | 1893 | O | ALA | A | 246 | 16.121 | 4.980 | 89.942 | 1.00 | 0.00 | xxxx | 1893 |
| ATOM | 1894 | CB | ALA | A | 246 | 16.582 | 8.001 | 88.924 | 1.00 | 0.00 | xxxx | 1894 |
| ATOM | 1895 | N | ILE | A | 247 | 18.113 | 5.690 | 90.717 | 1.00 | 0.00 | xxxx | 1895 |
| ATOM | 1896 | CA | ILE | A | 247 | 18.587 | 4.325 | 90.955 | 1.00 | 0.00 | xxxx | 1896 |
| ATOM | 1897 | C | ILE | A | 247 | 17.725 | 3.647 | 92.016 | 1.00 | 0.00 | xxxx | 1897 |
| ATOM | 1898 | O | ILE | A | 247 | 17.258 | 2.514 | 91.840 | 1.00 | 0.00 | xxxx | 1898 |
| ATOM | 1899 | CB | ILE | A | 247 | 20.070 | 4.327 | 91.361 | 1.00 | 0.00 | xxxx | 1899 |
| ATOM | 1900 | CG1 | ILE | A | 247 | 20.940 | 4.927 | 90.259 | 1.00 | 0.00 | xxxx | 1900 |
| ATOM | 1901 | CG2 | ILE | A | 247 | 20.533 | 2.903 | 91.692 | 1.00 | 0.00 | xxxx | 1901 |
| ATOM | 1902 | CD1 | ILE | A | 247 | 21.292 | 3.964 | 89.184 | 1.00 | 0.00 | xxxx | 1902 |
| ATOM | 1903 | N | LYS | A | 248 | 17.490 | 4.342 | 93.134 | 1.00 | 0.00 | xxxx | 1903 |
| ATOM | 1904 | CA | LYS | A | 248 | 16.681 | 3.779 | 94.214 | 1.00 | 0.00 | xxxx | 1904 |
| ATOM | 1905 | C | LYS | A | 248 | 15.234 | 3.559 | 93.784 | 1.00 | 0.00 | xxxx | 1905 |
| ATOM | 1906 | O | LYS | A | 248 | 14.592 | 2.608 | 94.245 | 1.00 | 0.00 | xxxx | 1906 |
| ATOM | 1907 | CB | LYS | A | 248 | 16.728 | 4.699 | 95.431 | 1.00 | 0.00 | xxxx | 1907 |
| ATOM | 1908 | CG | LYS | A | 248 | 18.108 | 4.863 | 96.041 | 1.00 | 0.00 | xxxx | 1908 |
| ATOM | 1909 | CD | LYS | A | 248 | 18.059 | 5.846 | 97.200 | 1.00 | 0.00 | xxxx | 1909 |
| ATOM | 1910 | CE | LYS | A | 248 | 19.406 | 5.968 | 97.881 | 1.00 | 0.00 | xxxx | 1910 |
| ATOM | 1911 | NZ | LYS | A | 248 | 19.339 | 6.962 | 98.994 | 1.00 | 0.00 | xxxx | 1911 |
| ATOM | 1912 | N | ASP | A | 249 | 14.709 | 4.416 | 92.901 | 1.00 | 0.00 | xxxx | 1912 |
| ATOM | 1913 | CA | ASP | A | 249 | 13.350 | 4.285 | 92.385 | 1.00 | 0.00 | xxxx | 1913 |
| ATOM | 1914 | C | ASP | A | 249 | 13.212 | 3.147 | 91.379 | 1.00 | 0.00 | xxxx | 1914 |
| ATOM | 1915 | O | ASP | A | 249 | 12.078 | 2.765 | 91.056 | 1.00 | 0.00 | xxxx | 1915 |
| ATOM | 1916 | CB | ASP | A | 249 | 12.911 | 5.580 | 91.688 | 1.00 | 0.00 | xxxx | 1916 |
| ATOM | 1917 | CG | ASP | A | 249 | 12.763 | 6.758 | 92.637 | 1.00 | 0.00 | xxxx | 1917 |
| ATOM | 1918 | OD1 | ASP | A | 249 | 12.696 | 6.548 | 93.866 | 1.00 | 0.00 | xxxx | 1918 |
| ATOM | 1919 | OD2 | ASP | A | 249 | 12.697 | 7.906 | 92.134 | 1.00 | 0.00 | xxxx | 1919 |
| ATOM | 1920 | N | GLY | A | 250 | 14.319 | 2.612 | 90.869 | 1.00 | 0.00 | xxxx | 1920 |
| ATOM | 1921 | CA | GLY | A | 250 | 14.265 | 1.554 | 89.881 | 1.00 | 0.00 | xxxx | 1921 |
| ATOM | 1922 | C | GLY | A | 250 | 14.164 | 2.022 | 88.441 | 1.00 | 0.00 | xxxx | 1922 |
| ATOM | 1923 | O | GLY | A | 250 | 14.071 | 1.180 | 87.540 | 1.00 | 0.00 | xxxx | 1923 |
| ATOM | 1924 | N | THR | A | 251 | 14.179 | 3.330 | 88.194 | 1.00 | 0.00 | xxxx | 1924 |
| ATOM | 1925 | CA | THR | A | 251 | 13.998 | 3.850 | 86.841 | 1.00 | 0.00 | xxxx | 1925 |
| ATOM | 1926 | C | THR | A | 251 | 15.314 | 4.102 | 86.118 | 1.00 | 0.00 | xxxx | 1926 |
| ATOM | 1927 | O | THR | A | 251 | 15.324 | 4.183 | 84.881 | 1.00 | 0.00 | xxxx | 1927 |
| ATOM | 1928 | CB | THR | A | 251 | 13.191 | 5.156 | 86.889 | 1.00 | 0.00 | xxxx | 1928 |
| ATOM | 1929 | OG1 | THR | A | 251 | 13.881 | 6.094 | 87.723 | 1.00 | 0.00 | xxxx | 1929 |
| ATOM | 1930 | CG2 | THR | A | 251 | 11.778 | 4.916 | 87.433 | 1.00 | 0.00 | xxxx | 1930 |
| ATOM | 1931 | N | LEU | A | 252 | 16.412 | 4.249 | 86.843 | 1.00 | 0.00 | xxxx | 1931 |
| ATOM | 1932 | CA | LEU | A | 252 | 17.732 | 4.400 | 86.245 | 1.00 | 0.00 | xxxx | 1932 |
| ATOM | 1933 | C | LEU | A | 252 | 18.501 | 3.128 | 86.565 | 1.00 | 0.00 | xxxx | 1933 |
| ATOM | 1934 | O | LEU | A | 252 | 18.624 | 2.759 | 87.735 | 1.00 | 0.00 | xxxx | 1934 |
| ATOM | 1935 | CB | LEU | A | 252 | 18.432 | 5.630 | 86.828 | 1.00 | 0.00 | xxxx | 1935 |
| ATOM | 1936 | CG | LEU | A | 252 | 19.763 | 6.044 | 86.193 | 1.00 | 0.00 | xxxx | 1936 |
| ATOM | 1937 | CD1 | LEU | A | 252 | 19.584 | 6.416 | 84.720 | 1.00 | 0.00 | xxxx | 1937 |
| ATOM | 1938 | CD2 | LEU | A | 252 | 20.375 | 7.224 | 86.963 | 1.00 | 0.00 | xxxx | 1938 |
| ATOM | 1939 | N | LEU | A | 253 | 18.960 | 2.428 | 85.521 | 1.00 | 0.00 | xxxx | 1939 |
| ATOM | 1940 | CA | LEU | A | 253 | 19.663 | 1.166 | 85.737 | 1.00 | 0.00 | xxxx | 1940 |
| ATOM | 1941 | C | LEU | A | 253 | 21.013 | 1.394 | 86.390 | 1.00 | 0.00 | xxxx | 1941 |
| ATOM | 1942 | O | LEU | A | 253 | 21.428 | 0.632 | 87.277 | 1.00 | 0.00 | xxxx | 1942 |
| ATOM | 1943 | CB | LEU | A | 253 | 19.849 | 0.445 | 84.402 | 1.00 | 0.00 | xxxx | 1943 |
| ATOM | 1944 | CG | LEU | A | 253 | 20.806 | −0.752 | 84.408 | 1.00 | 0.00 | xxxx | 1944 |
| ATOM | 1945 | CD1 | LEU | A | 253 | 20.224 | −1.883 | 85.239 | 1.00 | 0.00 | xxxx | 1945 |
| ATOM | 1946 | CD2 | LEU | A | 253 | 21.120 | −1.217 | 82.996 | 1.00 | 0.00 | xxxx | 1946 |
| ATOM | 1947 | N | GLY | A | 254 | 21.721 | 2.421 | 85.957 | 1.00 | 0.00 | xxxx | 1947 |
| ATOM | 1948 | CA | GLY | A | 254 | 23.032 | 2.651 | 86.518 | 1.00 | 0.00 | xxxx | 1948 |
| ATOM | 1949 | C | GLY | A | 254 | 23.542 | 4.002 | 86.097 | 1.00 | 0.00 | xxxx | 1949 |
| ATOM | 1950 | O | GLY | A | 254 | 23.017 | 4.642 | 85.180 | 1.00 | 0.00 | xxxx | 1950 |
| ATOM | 1951 | N | THR | A | 255 | 24.595 | 4.416 | 86.787 | 1.00 | 0.00 | xxxx | 1951 |
| ATOM | 1952 | CA | THR | A | 255 | 25.286 | 5.638 | 86.447 | 1.00 | 0.00 | xxxx | 1952 |
| ATOM | 1953 | C | THR | A | 255 | 26.754 | 5.460 | 86.803 | 1.00 | 0.00 | xxxx | 1953 |
| ATOM | 1954 | O | THR | A | 255 | 27.211 | 4.363 | 87.144 | 1.00 | 0.00 | xxxx | 1954 |
| ATOM | 1955 | CB | THR | A | 255 | 24.607 | 6.845 | 87.111 | 1.00 | 0.00 | xxxx | 1955 |
| ATOM | 1956 | OG1 | THR | A | 255 | 25.209 | 8.054 | 86.631 | 1.00 | 0.00 | xxxx | 1956 |
| ATOM | 1957 | CG2 | THR | A | 255 | 24.713 | 6.788 | 88.627 | 1.00 | 0.00 | xxxx | 1957 |
| ATOM | 1958 | N | VAL | A | 256 | 27.504 | 6.539 | 86.670 | 1.00 | 0.00 | xxxx | 1958 |
| ATOM | 1959 | CA | VAL | A | 256 | 28.924 | 6.541 | 86.956 | 1.00 | 0.00 | xxxx | 1959 |
| ATOM | 1960 | C | VAL | A | 256 | 29.165 | 7.740 | 87.852 | 1.00 | 0.00 | xxxx | 1960 |
| ATOM | 1961 | O | VAL | A | 256 | 28.821 | 8.862 | 87.478 | 1.00 | 0.00 | xxxx | 1961 |
| ATOM | 1962 | CB | VAL | A | 256 | 29.741 | 6.649 | 85.658 | 1.00 | 0.00 | xxxx | 1962 |
| ATOM | 1963 | CG1 | VAL | A | 256 | 31.235 | 6.627 | 85.977 | 1.00 | 0.00 | xxxx | 1963 |
| ATOM | 1964 | CG2 | VAL | A | 256 | 29.356 | 5.528 | 84.679 | 1.00 | 0.00 | xxxx | 1964 |
| ATOM | 1965 | N | LEU | A | 257 | 29.711 | 7.504 | 89.040 | 1.00 | 0.00 | xxxx | 1965 |
| ATOM | 1966 | CA | LEU | A | 257 | 30.015 | 8.606 | 89.941 | 1.00 | 0.00 | xxxx | 1966 |
| ATOM | 1967 | C | LEU | A | 257 | 31.080 | 9.495 | 89.321 | 1.00 | 0.00 | xxxx | 1967 |
| ATOM | 1968 | O | LEU | A | 257 | 32.188 | 9.032 | 89.022 | 1.00 | 0.00 | xxxx | 1968 |
| ATOM | 1969 | CB | LEU | A | 257 | 30.487 | 8.069 | 91.291 | 1.00 | 0.00 | xxxx | 1969 |
| ATOM | 1970 | CG | LEU | A | 257 | 30.976 | 9.158 | 92.256 | 1.00 | 0.00 | xxxx | 1970 |
| ATOM | 1971 | CD1 | LEU | A | 257 | 29.845 | 10.097 | 92.657 | 1.00 | 0.00 | xxxx | 1971 |
| ATOM | 1972 | CD2 | LEU | A | 257 | 31.614 | 8.517 | 93.481 | 1.00 | 0.00 | xxxx | 1972 |
| ATOM | 1973 | N | ASN | A | 258 | 30.718 | 10.764 | 89.095 | 1.00 | 0.00 | xxxx | 1973 |
| ATOM | 1974 | CA | ASN | A | 258 | 31.640 | 11.815 | 88.691 | 1.00 | 0.00 | xxxx | 1974 |
| ATOM | 1975 | C | ASN | A | 258 | 31.941 | 12.557 | 89.986 | 1.00 | 0.00 | xxxx | 1975 |
| ATOM | 1976 | O | ASN | A | 258 | 31.043 | 13.144 | 90.596 | 1.00 | 0.00 | xxxx | 1976 |
| ATOM | 1977 | CB | ASN | A | 258 | 30.968 | 12.719 | 87.655 | 1.00 | 0.00 | xxxx | 1977 |
| ATOM | 1978 | CG | ASN | A | 258 | 31.938 | 13.653 | 86.968 | 1.00 | 0.00 | xxxx | 1978 |
| ATOM | 1979 | OD1 | ASN | A | 258 | 33.139 | 13.611 | 87.231 | 1.00 | 0.00 | xxxx | 1979 |
| ATOM | 1980 | ND2 | ASN | A | 258 | 31.428 | 14.497 | 86.073 | 1.00 | 0.00 | xxxx | 1980 |
| ATOM | 1981 | N | ASP | A | 259 | 33.184 | 12.460 | 90.442 | 1.00 | 0.00 | xxxx | 1981 |
| ATOM | 1982 | CA | ASP | A | 259 | 33.543 | 12.754 | 91.829 | 1.00 | 0.00 | xxxx | 1982 |
| ATOM | 1983 | C | ASP | A | 259 | 33.747 | 14.260 | 91.986 | 1.00 | 0.00 | xxxx | 1983 |
| ATOM | 1984 | O | ASP | A | 259 | 34.868 | 14.780 | 91.900 | 1.00 | 0.00 | xxxx | 1984 |
| ATOM | 1985 | CB | ASP | A | 259 | 34.781 | 11.940 | 92.193 | 1.00 | 0.00 | xxxx | 1985 |
| ATOM | 1986 | CG | ASP | A | 259 | 35.185 | 12.071 | 93.647 | 1.00 | 0.00 | xxxx | 1986 |
| ATOM | 1987 | OD1 | ASP | A | 259 | 34.667 | 12.950 | 94.362 | 1.00 | 0.00 | xxxx | 1987 |
| ATOM | 1988 | OD2 | ASP | A | 259 | 36.067 | 11.285 | 94.061 | 1.00 | 0.00 | xxxx | 1988 |
| ATOM | 1989 | N | ALA | A | 260 | 32.639 | 14.961 | 92.251 | 1.00 | 0.00 | xxxx | 1989 |
| ATOM | 1990 | CA | ALA | A | 260 | 32.671 | 16.414 | 92.388 | 1.00 | 0.00 | xxxx | 1990 |
| ATOM | 1991 | C | ALA | A | 260 | 33.505 | 16.846 | 93.584 | 1.00 | 0.00 | xxxx | 1991 |
| ATOM | 1992 | O | ALA | A | 260 | 34.188 | 17.878 | 93.531 | 1.00 | 0.00 | xxxx | 1992 |
| ATOM | 1993 | CB | ALA | A | 260 | 31.246 | 16.943 | 92.547 | 1.00 | 0.00 | xxxx | 1993 |
| ATOM | 1994 | N | LYS | A | 261 | 33.456 | 16.085 | 94.680 | 1.00 | 0.00 | xxxx | 1994 |
| ATOM | 1995 | CA | LYS | A | 261 | 34.170 | 16.493 | 95.885 | 1.00 | 0.00 | xxxx | 1995 |
| ATOM | 1996 | C | LYS | A | 261 | 35.681 | 16.499 | 95.667 | 1.00 | 0.00 | xxxx | 1996 |
| ATOM | 1997 | O | LYS | A | 261 | 36.364 | 17.463 | 96.037 | 1.00 | 0.00 | xxxx | 1997 |
| ATOM | 1998 | CB | LYS | A | 261 | 33.771 | 15.608 | 97.064 | 1.00 | 0.00 | xxxx | 1998 |
| ATOM | 1999 | CG | LYS | A | 261 | 32.314 | 15.783 | 97.477 | 1.00 | 0.00 | xxxx | 1999 |
| ATOM | 2000 | CD | LYS | A | 261 | 31.976 | 14.931 | 98.695 | 1.00 | 0.00 | xxxx | 2000 |
| ATOM | 2001 | CE | LYS | A | 261 | 30.512 | 15.071 | 99.081 | 1.00 | 0.00 | xxxx | 2001 |
| ATOM | 2002 | NZ | LYS | A | 261 | 30.150 | 16.484 | 99.379 | 1.00 | 0.00 | xxxx | 2002 |
| ATOM | 2003 | N | ASN | A | 262 | 36.227 | 15.441 | 95.052 | 1.00 | 0.00 | xxxx | 2003 |
| ATOM | 2004 | CA | ASN | A | 262 | 37.666 | 15.428 | 94.815 | 1.00 | 0.00 | xxxx | 2004 |
| ATOM | 2005 | C | ASN | A | 262 | 38.082 | 16.350 | 93.674 | 1.00 | 0.00 | xxxx | 2005 |
| ATOM | 2006 | O | ASN | A | 262 | 39.187 | 16.901 | 93.721 | 1.00 | 0.00 | xxxx | 2006 |
| ATOM | 2007 | CB | ASN | A | 262 | 38.198 | 14.002 | 94.643 | 1.00 | 0.00 | xxxx | 2007 |
| ATOM | 2008 | CG | ASN | A | 262 | 38.342 | 13.280 | 95.971 | 1.00 | 0.00 | xxxx | 2008 |
| ATOM | 2009 | OD1 | ASN | A | 262 | 39.094 | 13.708 | 96.849 | 1.00 | 0.00 | xxxx | 2009 |
| ATOM | 2010 | ND2 | ASN | A | 262 | 37.614 | 12.190 | 96.125 | 1.00 | 0.00 | xxxx | 2010 |
| ATOM | 2011 | N | GLN | A | 263 | 37.226 | 16.564 | 92.668 | 1.00 | 0.00 | xxxx | 2011 |
| ATOM | 2012 | CA | GLN | A | 263 | 37.565 | 17.566 | 91.657 | 1.00 | 0.00 | xxxx | 2012 |
| ATOM | 2013 | C | GLN | A | 263 | 37.590 | 18.958 | 92.268 | 1.00 | 0.00 | xxxx | 2013 |
| ATOM | 2014 | O | GLN | A | 263 | 38.479 | 19.759 | 91.957 | 1.00 | 0.00 | xxxx | 2014 |
| ATOM | 2015 | CB | GLN | A | 263 | 36.608 | 17.491 | 90.463 | 1.00 | 0.00 | xxxx | 2015 |
| ATOM | 2016 | CG | GLN | A | 263 | 36.799 | 16.209 | 89.647 | 1.00 | 0.00 | xxxx | 2016 |
| ATOM | 2017 | CD | GLN | A | 263 | 35.778 | 16.030 | 88.551 | 1.00 | 0.00 | xxxx | 2017 |
| ATOM | 2018 | OE1 | GLN | A | 263 | 35.494 | 16.960 | 87.787 | 1.00 | 0.00 | xxxx | 2018 |
| ATOM | 2019 | NE2 | GLN | A | 263 | 35.240 | 14.823 | 88.445 | 1.00 | 0.00 | xxxx | 2019 |
| ATOM | 2020 | N | ALA | A | 264 | 36.644 | 19.248 | 93.163 | 1.00 | 0.00 | xxxx | 2020 |
| ATOM | 2021 | CA | ALA | A | 264 | 36.625 | 20.545 | 93.830 | 1.00 | 0.00 | xxxx | 2021 |
| ATOM | 2022 | C | ALA | A | 264 | 37.857 | 20.733 | 94.698 | 1.00 | 0.00 | xxxx | 2022 |
| ATOM | 2023 | O | ALA | A | 264 | 38.449 | 21.820 | 94.715 | 1.00 | 0.00 | xxxx | 2023 |
| ATOM | 2024 | CB | ALA | A | 264 | 35.364 | 20.685 | 94.682 | 1.00 | 0.00 | xxxx | 2024 |
| ATOM | 2025 | N | LYS | A | 265 | 38.251 | 19.688 | 95.441 | 1.00 | 0.00 | xxxx | 2025 |
| ATOM | 2026 | CA | LYS | A | 265 | 39.406 | 19.798 | 96.327 | 1.00 | 0.00 | xxxx | 2026 |
| ATOM | 2027 | C | LYS | A | 265 | 40.695 | 19.981 | 95.537 | 1.00 | 0.00 | xxxx | 2027 |
| ATOM | 2028 | O | LYS | A | 265 | 41.549 | 20.796 | 95.911 | 1.00 | 0.00 | xxxx | 2028 |
| ATOM | 2029 | CB | LYS | A | 265 | 39.493 | 18.569 | 97.235 | 1.00 | 0.00 | xxxx | 2029 |
| ATOM | 2030 | CG | LYS | A | 265 | 40.520 | 18.700 | 98.345 | 1.00 | 0.00 | xxxx | 2030 |
| ATOM | 2031 | CD | LYS | A | 265 | 40.489 | 17.514 | 99.304 | 1.00 | 0.00 | xxxx | 2031 |
| ATOM | 2032 | CE | LYS | A | 265 | 41.232 | 17.846 | 100.589 | 1.00 | 0.00 | xxxx | 2032 |
| ATOM | 2033 | NZ | LYS | A | 265 | 42.514 | 18.556 | 100.318 | 1.00 | 0.00 | xxxx | 2033 |
| ATOM | 2034 | N | ALA | A | 266 | 40.855 | 19.237 | 94.440 | 1.00 | 0.00 | xxxx | 2034 |
| ATOM | 2035 | CA | ALA | A | 266 | 42.049 | 19.392 | 93.619 | 1.00 | 0.00 | xxxx | 2035 |
| ATOM | 2036 | C | ALA | A | 266 | 42.104 | 20.779 | 92.984 | 1.00 | 0.00 | xxxx | 2036 |
| ATOM | 2037 | O | ALA | A | 266 | 43.165 | 21.411 | 92.952 | 1.00 | 0.00 | xxxx | 2037 |
| ATOM | 2038 | CB | ALA | A | 266 | 42.111 | 18.298 | 92.553 | 1.00 | 0.00 | xxxx | 2038 |
| ATOM | 2039 | N | THR | A | 267 | 40.959 | 21.279 | 92.503 | 1.00 | 0.00 | xxxx | 2039 |
| ATOM | 2040 | CA | THR | A | 267 | 40.914 | 22.606 | 91.884 | 1.00 | 0.00 | xxxx | 2040 |
| ATOM | 2041 | C | THR | A | 267 | 41.245 | 23.686 | 92.901 | 1.00 | 0.00 | xxxx | 2041 |
| ATOM | 2042 | O | THR | A | 267 | 42.079 | 24.570 | 92.647 | 1.00 | 0.00 | xxxx | 2042 |
| ATOM | 2043 | CB | THR | A | 267 | 39.529 | 22.864 | 91.293 | 1.00 | 0.00 | xxxx | 2043 |
| ATOM | 2044 | OG1 | THR | A | 267 | 39.235 | 21.873 | 90.304 | 1.00 | 0.00 | xxxx | 2044 |
| ATOM | 2045 | CG2 | THR | A | 267 | 39.483 | 24.234 | 90.627 | 1.00 | 0.00 | xxxx | 2045 |
| ATOM | 2046 | N | PHE | A | 268 | 40.600 | 23.633 | 94.067 | 1.00 | 0.00 | xxxx | 2046 |
| ATOM | 2047 | CA | PHE | A | 268 | 40.922 | 24.609 | 95.096 | 1.00 | 0.00 | xxxx | 2047 |
| ATOM | 2048 | C | PHE | A | 268 | 42.388 | 24.525 | 95.490 | 1.00 | 0.00 | xxxx | 2048 |
| ATOM | 2049 | O | PHE | A | 268 | 43.061 | 25.554 | 95.609 | 1.00 | 0.00 | xxxx | 2049 |
| ATOM | 2050 | CB | PHE | A | 268 | 40.052 | 24.460 | 96.344 | 1.00 | 0.00 | xxxx | 2050 |
| ATOM | 2051 | CG | PHE | A | 268 | 40.498 | 25.370 | 97.436 | 1.00 | 0.00 | xxxx | 2051 |
| ATOM | 2052 | CD1 | PHE | A | 268 | 40.176 | 26.712 | 97.382 | 1.00 | 0.00 | xxxx | 2052 |
| ATOM | 2053 | CD2 | PHE | A | 268 | 41.316 | 24.920 | 98.463 | 1.00 | 0.00 | xxxx | 2053 |
| ATOM | 2054 | CE1 | PHE | A | 268 | 40.627 | 27.585 | 98.350 | 1.00 | 0.00 | xxxx | 2054 |
| ATOM | 2055 | CE2 | PHE | A | 268 | 41.770 | 25.791 | 99.436 | 1.00 | 0.00 | xxxx | 2055 |
| ATOM | 2056 | CZ | PHE | A | 268 | 41.430 | 27.125 | 99.382 | 1.00 | 0.00 | xxxx | 2056 |
| ATOM | 2057 | N | ASN | A | 269 | 42.891 | 23.307 | 95.749 | 1.00 | 0.00 | xxxx | 2057 |
| ATOM | 2058 | CA | ASN | A | 269 | 44.260 | 23.169 | 96.239 | 1.00 | 0.00 | xxxx | 2058 |
| ATOM | 2059 | C | ASN | A | 269 | 45.260 | 23.728 | 95.236 | 1.00 | 0.00 | xxxx | 2059 |
| ATOM | 2060 | O | ASN | A | 269 | 46.225 | 24.396 | 95.627 | 1.00 | 0.00 | xxxx | 2060 |
| ATOM | 2061 | CB | ASN | A | 269 | 44.596 | 21.705 | 96.537 | 1.00 | 0.00 | xxxx | 2061 |
| ATOM | 2062 | CG | ASN | A | 269 | 43.939 | 21.179 | 97.797 | 1.00 | 0.00 | xxxx | 2062 |
| ATOM | 2063 | OD1 | ASN | A | 269 | 44.044 | 19.987 | 98.102 | 1.00 | 0.00 | xxxx | 2063 |
| ATOM | 2064 | ND2 | ASN | A | 269 | 43.253 | 22.045 | 98.526 | 1.00 | 0.00 | xxxx | 2064 |
| ATOM | 2065 | N | ILE | A | 270 | 45.040 | 23.483 | 93.939 | 1.00 | 0.00 | xxxx | 2065 |
| ATOM | 2066 | CA | ILE | A | 270 | 45.923 | 24.058 | 92.923 | 1.00 | 0.00 | xxxx | 2066 |
| ATOM | 2067 | C | ILE | A | 270 | 45.859 | 25.579 | 92.962 | 1.00 | 0.00 | xxxx | 2067 |
| ATOM | 2068 | O | ILE | A | 270 | 46.893 | 26.259 | 92.973 | 1.00 | 0.00 | xxxx | 2068 |
| ATOM | 2069 | CB | ILE | A | 270 | 45.571 | 23.517 | 91.527 | 1.00 | 0.00 | xxxx | 2069 |
| ATOM | 2070 | CG1 | ILE | A | 270 | 46.007 | 22.072 | 91.408 | 1.00 | 0.00 | xxxx | 2070 |
| ATOM | 2071 | CG2 | ILE | A | 270 | 46.236 | 24.370 | 90.425 | 1.00 | 0.00 | xxxx | 2071 |
| ATOM | 2072 | CD1 | ILE | A | 270 | 45.457 | 21.393 | 90.169 | 1.00 | 0.00 | xxxx | 2072 |
| ATOM | 2073 | N | ALA | A | 271 | 44.645 | 26.142 | 92.967 | 1.00 | 0.00 | xxxx | 2073 |
| ATOM | 2074 | CA | ALA | A | 271 | 44.510 | 27.599 | 92.969 | 1.00 | 0.00 | xxxx | 2074 |
| ATOM | 2075 | C | ALA | A | 271 | 45.108 | 28.206 | 94.229 | 1.00 | 0.00 | xxxx | 2075 |
| ATOM | 2076 | O | ALA | A | 271 | 45.705 | 29.292 | 94.188 | 1.00 | 0.00 | xxxx | 2076 |
| ATOM | 2077 | CB | ALA | A | 271 | 43.038 | 27.994 | 92.836 | 1.00 | 0.00 | xxxx | 2077 |
| ATOM | 2078 | N | TYR | A | 272 | 44.980 | 27.506 | 95.354 | 1.00 | 0.00 | xxxx | 2078 |
| ATOM | 2079 | CA | TYR | A | 272 | 45.485 | 28.004 | 96.629 | 1.00 | 0.00 | xxxx | 2079 |
| ATOM | 2080 | C | TYR | A | 272 | 47.012 | 28.021 | 96.651 | 1.00 | 0.00 | xxxx | 2080 |
| ATOM | 2081 | O | TYR | A | 272 | 47.624 | 28.977 | 97.147 | 1.00 | 0.00 | xxxx | 2081 |
| ATOM | 2082 | CB | TYR | A | 272 | 44.909 | 27.125 | 97.741 | 1.00 | 0.00 | xxxx | 2082 |
| ATOM | 2083 | CG | TYR | A | 272 | 45.268 | 27.495 | 99.162 | 1.00 | 0.00 | xxxx | 2083 |
| ATOM | 2084 | CD1 | TYR | A | 272 | 44.912 | 28.721 | 99.698 | 1.00 | 0.00 | xxxx | 2084 |
| ATOM | 2085 | CD2 | TYR | A | 272 | 45.916 | 26.584 | 99.983 | 1.00 | 0.00 | xxxx | 2085 |
| ATOM | 2086 | CE1 | TYR | A | 272 | 45.223 | 29.047 | 101.014 | 1.00 | 0.00 | xxxx | 2086 |
| ATOM | 2087 | CE2 | TYR | A | 272 | 46.228 | 26.900 | 101.296 | 1.00 | 0.00 | xxxx | 2087 |
| ATOM | 2088 | CZ | TYR | A | 272 | 45.876 | 28.133 | 101.799 | 1.00 | 0.00 | xxxx | 2088 |
| ATOM | 2089 | OH | TYR | A | 272 | 46.183 | 28.458 | 103.102 | 1.00 | 0.00 | xxxx | 2089 |
| ATOM | 2090 | N | GLU | A | 273 | 47.651 | 26.968 | 96.126 | 1.00 | 0.00 | xxxx | 2090 |
| ATOM | 2091 | CA | GLU | A | 273 | 49.107 | 26.974 | 96.051 | 1.00 | 0.00 | xxxx | 2091 |
| ATOM | 2092 | C | GLU | A | 273 | 49.592 | 28.062 | 95.104 | 1.00 | 0.00 | xxxx | 2092 |
| ATOM | 2093 | O | GLU | A | 273 | 50.506 | 28.826 | 95.437 | 1.00 | 0.00 | xxxx | 2093 |
| ATOM | 2094 | CB | GLU | A | 273 | 49.621 | 25.598 | 95.622 | 1.00 | 0.00 | xxxx | 2094 |
| ATOM | 2095 | CG | GLU | A | 273 | 49.298 | 24.482 | 96.611 | 1.00 | 0.00 | xxxx | 2095 |
| ATOM | 2096 | CD | GLU | A | 273 | 49.896 | 24.718 | 97.992 | 1.00 | 0.00 | xxxx | 2096 |
| ATOM | 2097 | OE1 | GLU | A | 273 | 51.089 | 25.073 | 98.078 | 1.00 | 0.00 | xxxx | 2097 |
| ATOM | 2098 | OE2 | GLU | A | 273 | 49.168 | 24.555 | 98.993 | 1.00 | 0.00 | xxxx | 2098 |
| ATOM | 2099 | N | LEU | A | 274 | 48.968 | 28.165 | 93.928 | 1.00 | 0.00 | xxxx | 2099 |
| ATOM | 2100 | CA | LEU | A | 274 | 49.361 | 29.188 | 92.963 | 1.00 | 0.00 | xxxx | 2100 |
| ATOM | 2101 | C | LEU | A | 274 | 49.151 | 30.589 | 93.519 | 1.00 | 0.00 | xxxx | 2101 |
| ATOM | 2102 | O | LEU | A | 274 | 49.959 | 31.488 | 93.267 | 1.00 | 0.00 | xxxx | 2102 |
| ATOM | 2103 | CB | LEU | A | 274 | 48.577 | 29.004 | 91.666 | 1.00 | 0.00 | xxxx | 2103 |
| ATOM | 2104 | CG | LEU | A | 274 | 48.907 | 27.761 | 90.837 | 1.00 | 0.00 | xxxx | 2104 |
| ATOM | 2105 | CD1 | LEU | A | 274 | 47.868 | 27.549 | 89.742 | 1.00 | 0.00 | xxxx | 2105 |
| ATOM | 2106 | CD2 | LEU | A | 274 | 50.315 | 27.857 | 90.249 | 1.00 | 0.00 | xxxx | 2106 |
| ATOM | 2107 | N | ALA | A | 275 | 48.073 | 30.794 | 94.281 | 1.00 | 0.00 | xxxx | 2107 |
| ATOM | 2108 | CA | ALA | A | 275 | 47.819 | 32.092 | 94.897 | 1.00 | 0.00 | xxxx | 2108 |
| ATOM | 2109 | C | ALA | A | 275 | 48.931 | 32.488 | 95.856 | 1.00 | 0.00 | xxxx | 2109 |
| ATOM | 2110 | O | ALA | A | 275 | 49.166 | 33.683 | 96.076 | 1.00 | 0.00 | xxxx | 2110 |
| ATOM | 2111 | CB | ALA | A | 275 | 46.482 | 32.054 | 95.636 | 1.00 | 0.00 | xxxx | 2111 |
| ATOM | 2112 | N | GLN | A | 276 | 49.604 | 31.510 | 96.454 | 1.00 | 0.00 | xxxx | 2112 |
| ATOM | 2113 | CA | GLN | A | 276 | 50.722 | 31.741 | 97.361 | 1.00 | 0.00 | xxxx | 2113 |
| ATOM | 2114 | C | GLN | A | 276 | 52.057 | 31.817 | 96.634 | 1.00 | 0.00 | xxxx | 2114 |
| ATOM | 2115 | O | GLN | A | 276 | 53.094 | 31.981 | 97.288 | 1.00 | 0.00 | xxxx | 2115 |
| ATOM | 2116 | CB | GLN | A | 276 | 50.784 | 30.628 | 98.409 | 1.00 | 0.00 | xxxx | 2116 |
| ATOM | 2117 | CG | GLN | A | 276 | 49.579 | 30.556 | 99.333 | 1.00 | 0.00 | xxxx | 2117 |
| ATOM | 2118 | CD | GLN | A | 276 | 49.603 | 29.331 | 100.233 | 1.00 | 0.00 | xxxx | 2118 |
| ATOM | 2119 | OE1 | GLN | A | 276 | 48.887 | 28.352 | 100.001 | 1.00 | 0.00 | xxxx | 2119 |
| ATOM | 2120 | NE2 | GLN | A | 276 | 50.426 | 29.386 | 101.275 | 1.00 | 0.00 | xxxx | 2120 |
| ATOM | 2121 | N | GLY | A | 277 | 52.056 | 31.705 | 95.308 | 1.00 | 0.00 | xxxx | 2121 |
| ATOM | 2122 | CA | GLY | A | 277 | 53.294 | 31.680 | 94.550 | 1.00 | 0.00 | xxxx | 2122 |
| ATOM | 2123 | C | GLY | A | 277 | 54.045 | 30.369 | 94.618 | 1.00 | 0.00 | xxxx | 2123 |
| ATOM | 2124 | O | GLY | A | 277 | 55.264 | 30.349 | 94.415 | 1.00 | 0.00 | xxxx | 2124 |
| ATOM | 2125 | N | ILE | A | 278 | 53.350 | 29.266 | 94.886 | 1.00 | 0.00 | xxxx | 2125 |
| ATOM | 2126 | CA | ILE | A | 278 | 53.963 | 27.956 | 95.076 | 1.00 | 0.00 | xxxx | 2126 |
| ATOM | 2127 | C | ILE | A | 278 | 53.561 | 27.072 | 93.905 | 1.00 | 0.00 | xxxx | 2127 |
| ATOM | 2128 | O | ILE | A | 278 | 52.384 | 27.034 | 93.527 | 1.00 | 0.00 | xxxx | 2128 |
| ATOM | 2129 | CB | ILE | A | 278 | 53.511 | 27.336 | 96.409 | 1.00 | 0.00 | xxxx | 2129 |
| ATOM | 2130 | CG1 | ILE | A | 278 | 53.891 | 28.251 | 97.577 | 1.00 | 0.00 | xxxx | 2130 |
| ATOM | 2131 | CG2 | ILE | A | 278 | 54.097 | 25.940 | 96.585 | 1.00 | 0.00 | xxxx | 2131 |
| ATOM | 2132 | CD1 | ILE | A | 278 | 53.323 | 27.804 | 98.910 | 1.00 | 0.00 | xxxx | 2132 |
| ATOM | 2133 | N | THR | A | 279 | 54.528 | 26.350 | 93.339 | 1.00 | 0.00 | xxxx | 2133 |
| ATOM | 2134 | CA | THR | A | 279 | 54.222 | 25.468 | 92.210 | 1.00 | 0.00 | xxxx | 2134 |
| ATOM | 2135 | C | THR | A | 279 | 53.522 | 24.206 | 92.708 | 1.00 | 0.00 | xxxx | 2135 |
| ATOM | 2136 | O | THR | A | 279 | 54.030 | 23.542 | 93.616 | 1.00 | 0.00 | xxxx | 2136 |
| ATOM | 2137 | CB | THR | A | 279 | 55.507 | 25.085 | 91.474 | 1.00 | 0.00 | xxxx | 2137 |
| ATOM | 2138 | OG1 | THR | A | 279 | 56.136 | 26.266 | 90.970 | 1.00 | 0.00 | xxxx | 2138 |
| ATOM | 2139 | CG2 | THR | A | 279 | 55.206 | 24.153 | 90.305 | 1.00 | 0.00 | xxxx | 2139 |
| ATOM | 2140 | N | PRO | A | 280 | 52.372 | 23.844 | 92.141 | 1.00 | 0.00 | xxxx | 2140 |
| ATOM | 2141 | CA | PRO | A | 280 | 51.693 | 22.611 | 92.562 | 1.00 | 0.00 | xxxx | 2141 |
| ATOM | 2142 | C | PRO | A | 280 | 52.570 | 21.376 | 92.401 | 1.00 | 0.00 | xxxx | 2142 |
| ATOM | 2143 | O | PRO | A | 280 | 53.312 | 21.237 | 91.426 | 1.00 | 0.00 | xxxx | 2143 |
| ATOM | 2144 | CB | PRO | A | 280 | 50.483 | 22.551 | 91.626 | 1.00 | 0.00 | xxxx | 2144 |
| ATOM | 2145 | CG | PRO | A | 280 | 50.192 | 23.983 | 91.318 | 1.00 | 0.00 | xxxx | 2145 |
| ATOM | 2146 | CD | PRO | A | 280 | 51.543 | 24.644 | 91.222 | 1.00 | 0.00 | xxxx | 2146 |
| ATOM | 2147 | N | THR | A | 281 | 52.488 | 20.487 | 93.396 | 1.00 | 0.00 | xxxx | 2147 |
| ATOM | 2148 | CA | THR | A | 281 | 53.091 | 19.158 | 93.353 | 1.00 | 0.00 | xxxx | 2148 |
| ATOM | 2149 | C | THR | A | 281 | 52.078 | 18.181 | 93.927 | 1.00 | 0.00 | xxxx | 2149 |
| ATOM | 2150 | O | THR | A | 281 | 51.140 | 18.577 | 94.621 | 1.00 | 0.00 | xxxx | 2150 |
| ATOM | 2151 | CB | THR | A | 281 | 54.352 | 19.067 | 94.218 | 1.00 | 0.00 | xxxx | 2151 |
| ATOM | 2152 | OG1 | THR | A | 281 | 53.984 | 19.227 | 95.593 | 1.00 | 0.00 | xxxx | 2152 |
| ATOM | 2153 | CG2 | THR | A | 281 | 55.373 | 20.140 | 93.839 | 1.00 | 0.00 | xxxx | 2153 |
| ATOM | 2154 | N | LYS | A | 282 | 52.285 | 16.887 | 93.664 | 1.00 | 0.00 | xxxx | 2154 |
| ATOM | 2155 | CA | LYS | A | 282 | 51.404 | 15.889 | 94.267 | 1.00 | 0.00 | xxxx | 2155 |
| ATOM | 2156 | C | LYS | A | 282 | 51.395 | 16.013 | 95.786 | 1.00 | 0.00 | xxxx | 2156 |
| ATOM | 2157 | O | LYS | A | 282 | 50.346 | 15.855 | 96.420 | 1.00 | 0.00 | xxxx | 2157 |
| ATOM | 2158 | CB | LYS | A | 282 | 51.817 | 14.480 | 93.832 | 1.00 | 0.00 | xxxx | 2158 |
| ATOM | 2159 | CG | LYS | A | 282 | 51.023 | 13.369 | 94.510 | 1.00 | 0.00 | xxxx | 2159 |
| ATOM | 2160 | CD | LYS | A | 282 | 51.461 | 11.990 | 94.042 | 1.00 | 0.00 | xxxx | 2160 |
| ATOM | 2161 | CE | LYS | A | 282 | 50.575 | 10.900 | 94.638 | 1.00 | 0.00 | xxxx | 2161 |
| ATOM | 2162 | NZ | LYS | A | 282 | 50.903 | 9.547 | 94.098 | 1.00 | 0.00 | xxxx | 2162 |
| ATOM | 2163 | N | ASP | A | 283 | 52.544 | 16.331 | 96.386 | 1.00 | 0.00 | xxxx | 2163 |
| ATOM | 2164 | CA | ASP | A | 283 | 52.605 | 16.451 | 97.839 | 1.00 | 0.00 | xxxx | 2164 |
| ATOM | 2165 | C | ASP | A | 283 | 51.787 | 17.636 | 98.342 | 1.00 | 0.00 | xxxx | 2165 |
| ATOM | 2166 | O | ASP | A | 283 | 51.107 | 17.529 | 99.369 | 1.00 | 0.00 | xxxx | 2166 |
| ATOM | 2167 | CB | ASP | A | 283 | 54.055 | 16.583 | 98.304 | 1.00 | 0.00 | xxxx | 2167 |
| ATOM | 2168 | CG | ASP | A | 283 | 54.833 | 15.287 | 98.184 | 1.00 | 0.00 | xxxx | 2168 |
| ATOM | 2169 | OD1 | ASP | A | 283 | 54.208 | 14.208 | 98.131 | 1.00 | 0.00 | xxxx | 2169 |
| ATOM | 2170 | OD2 | ASP | A | 283 | 56.081 | 15.351 | 98.155 | 1.00 | 0.00 | xxxx | 2170 |
| ATOM | 2171 | N | ASN | A | 284 | 51.834 | 18.777 | 97.647 | 1.00 | 0.00 | xxxx | 2171 |
| ATOM | 2172 | CA | ASN | A | 284 | 51.185 | 19.963 | 98.195 | 1.00 | 0.00 | xxxx | 2172 |
| ATOM | 2173 | C | ASN | A | 284 | 49.744 | 20.169 | 97.733 | 1.00 | 0.00 | xxxx | 2173 |
| ATOM | 2174 | O | ASN | A | 284 | 49.050 | 21.012 | 98.313 | 1.00 | 0.00 | xxxx | 2174 |
| ATOM | 2175 | CB | ASN | A | 284 | 52.050 | 21.233 | 98.047 | 1.00 | 0.00 | xxxx | 2175 |
| ATOM | 2176 | CG | ASN | A | 284 | 52.213 | 21.691 | 96.609 | 1.00 | 0.00 | xxxx | 2176 |
| ATOM | 2177 | OD1 | ASN | A | 284 | 51.340 | 21.487 | 95.771 | 1.00 | 0.00 | xxxx | 2177 |
| ATOM | 2178 | ND2 | ASN | A | 284 | 53.339 | 22.348 | 96.327 | 1.00 | 0.00 | xxxx | 2178 |
| ATOM | 2179 | N | ILE | A | 285 | 49.265 | 19.429 | 96.732 | 1.00 | 0.00 | xxxx | 2179 |
| ATOM | 2180 | CA | ILE | A | 285 | 47.845 | 19.447 | 96.394 | 1.00 | 0.00 | xxxx | 2180 |
| ATOM | 2181 | C | ILE | A | 285 | 47.131 | 18.152 | 96.753 | 1.00 | 0.00 | xxxx | 2181 |
| ATOM | 2182 | O | ILE | A | 285 | 45.892 | 18.151 | 96.822 | 1.00 | 0.00 | xxxx | 2182 |
| ATOM | 2183 | CB | ILE | A | 285 | 47.552 | 19.846 | 94.929 | 1.00 | 0.00 | xxxx | 2183 |
| ATOM | 2184 | CG1 | ILE | A | 285 | 47.964 | 18.729 | 93.966 | 1.00 | 0.00 | xxxx | 2184 |
| ATOM | 2185 | CG2 | ILE | A | 285 | 48.214 | 21.176 | 94.591 | 1.00 | 0.00 | xxxx | 2185 |
| ATOM | 2186 | CD1 | ILE | A | 285 | 47.381 | 18.877 | 92.565 | 1.00 | 0.00 | xxxx | 2186 |
| ATOM | 2187 | N | GLY | A | 286 | 47.853 | 17.057 | 96.966 | 1.00 | 0.00 | xxxx | 2187 |
| ATOM | 2188 | CA | GLY | A | 286 | 47.239 | 15.817 | 97.401 | 1.00 | 0.00 | xxxx | 2188 |
| ATOM | 2189 | C | GLY | A | 286 | 46.744 | 14.889 | 96.311 | 1.00 | 0.00 | xxxx | 2189 |
| ATOM | 2190 | O | GLY | A | 286 | 45.989 | 13.958 | 96.620 | 1.00 | 0.00 | xxxx | 2190 |
| ATOM | 2191 | N | TYR | A | 287 | 47.117 | 15.123 | 95.049 | 1.00 | 0.00 | xxxx | 2191 |
| ATOM | 2192 | CA | TYR | A | 287 | 46.656 | 14.329 | 93.912 | 1.00 | 0.00 | xxxx | 2192 |
| ATOM | 2193 | C | TYR | A | 287 | 47.783 | 14.234 | 92.898 | 1.00 | 0.00 | xxxx | 2193 |
| ATOM | 2194 | O | TYR | A | 287 | 48.562 | 15.175 | 92.738 | 1.00 | 0.00 | xxxx | 2194 |
| ATOM | 2195 | CB | TYR | A | 287 | 45.445 | 14.977 | 93.206 | 1.00 | 0.00 | xxxx | 2195 |
| ATOM | 2196 | CG | TYR | A | 287 | 44.247 | 15.146 | 94.110 | 1.00 | 0.00 | xxxx | 2196 |
| ATOM | 2197 | CD1 | TYR | A | 287 | 44.120 | 16.268 | 94.922 | 1.00 | 0.00 | xxxx | 2197 |
| ATOM | 2198 | CD2 | TYR | A | 287 | 43.247 | 14.173 | 94.173 | 1.00 | 0.00 | xxxx | 2198 |
| ATOM | 2199 | CE1 | TYR | A | 287 | 43.045 | 16.414 | 95.778 | 1.00 | 0.00 | xxxx | 2199 |
| ATOM | 2200 | CE2 | TYR | A | 287 | 42.169 | 14.310 | 95.020 | 1.00 | 0.00 | xxxx | 2200 |
| ATOM | 2201 | CZ | TYR | A | 287 | 42.072 | 15.436 | 95.823 | 1.00 | 0.00 | xxxx | 2201 |
| ATOM | 2202 | OH | TYR | A | 287 | 41.016 | 15.600 | 96.685 | 1.00 | 0.00 | xxxx | 2202 |
| ATOM | 2203 | N | ASP | A | 288 | 47.849 | 13.100 | 92.198 | 1.00 | 0.00 | xxxx | 2203 |
| ATOM | 2204 | CA | ASP | A | 288 | 48.789 | 12.962 | 91.092 | 1.00 | 0.00 | xxxx | 2204 |
| ATOM | 2205 | C | ASP | A | 288 | 48.519 | 13.995 | 90.009 | 1.00 | 0.00 | xxxx | 2205 |
| ATOM | 2206 | O | ASP | A | 288 | 47.367 | 14.248 | 89.636 | 1.00 | 0.00 | xxxx | 2206 |
| ATOM | 2207 | CB | ASP | A | 288 | 48.654 | 11.590 | 90.440 | 1.00 | 0.00 | xxxx | 2207 |
| ATOM | 2208 | CG | ASP | A | 288 | 49.223 | 10.483 | 91.279 | 1.00 | 0.00 | xxxx | 2208 |
| ATOM | 2209 | OD1 | ASP | A | 288 | 50.454 | 10.475 | 91.494 | 1.00 | 0.00 | xxxx | 2209 |
| ATOM | 2210 | OD2 | ASP | A | 288 | 48.442 | 9.600 | 91.691 | 1.00 | 0.00 | xxxx | 2210 |
| ATOM | 2211 | N | ILE | A | 289 | 49.601 | 14.562 | 89.485 | 1.00 | 0.00 | xxxx | 2211 |
| ATOM | 2212 | CA | ILE | A | 289 | 49.578 | 15.483 | 88.353 | 1.00 | 0.00 | xxxx | 2212 |
| ATOM | 2213 | C | ILE | A | 289 | 50.142 | 14.742 | 87.150 | 1.00 | 0.00 | xxxx | 2213 |
| ATOM | 2214 | O | ILE | A | 289 | 51.215 | 14.129 | 87.235 | 1.00 | 0.00 | xxxx | 2214 |
| ATOM | 2215 | CB | ILE | A | 289 | 50.395 | 16.751 | 88.654 | 1.00 | 0.00 | xxxx | 2215 |
| ATOM | 2216 | CG1 | ILE | A | 289 | 49.854 | 17.448 | 89.904 | 1.00 | 0.00 | xxxx | 2216 |
| ATOM | 2217 | CG2 | ILE | A | 289 | 50.393 | 17.699 | 87.452 | 1.00 | 0.00 | xxxx | 2217 |
| ATOM | 2218 | CD1 | ILE | A | 289 | 50.751 | 18.579 | 90.397 | 1.00 | 0.00 | xxxx | 2218 |
| ATOM | 2219 | N | THR | A | 290 | 49.412 | 14.779 | 86.040 | 1.00 | 0.00 | xxxx | 2219 |
| ATOM | 2220 | CA | THR | A | 290 | 49.764 | 14.060 | 84.824 | 1.00 | 0.00 | xxxx | 2220 |
| ATOM | 2221 | C | THR | A | 290 | 50.168 | 15.065 | 83.756 | 1.00 | 0.00 | xxxx | 2221 |
| ATOM | 2222 | O | THR | A | 290 | 49.539 | 16.124 | 83.623 | 1.00 | 0.00 | xxxx | 2222 |
| ATOM | 2223 | CB | THR | A | 290 | 48.550 | 13.264 | 84.336 | 1.00 | 0.00 | xxxx | 2223 |
| ATOM | 2224 | OG1 | THR | A | 290 | 48.119 | 12.361 | 85.365 | 1.00 | 0.00 | xxxx | 2224 |
| ATOM | 2225 | CG2 | THR | A | 290 | 48.887 | 12.467 | 83.098 | 1.00 | 0.00 | xxxx | 2225 |
| ATOM | 2226 | N | ASP | A | 291 | 51.224 | 14.740 | 83.002 | 1.00 | 0.00 | xxxx | 2226 |
| ATOM | 2227 | CA | ASP | A | 291 | 51.724 | 15.614 | 81.931 | 1.00 | 0.00 | xxxx | 2227 |
| ATOM | 2228 | C | ASP | A | 291 | 52.036 | 17.022 | 82.437 | 1.00 | 0.00 | xxxx | 2228 |
| ATOM | 2229 | O | ASP | A | 291 | 51.957 | 17.998 | 81.684 | 1.00 | 0.00 | xxxx | 2229 |
| ATOM | 2230 | CB | ASP | A | 291 | 50.750 | 15.669 | 80.746 | 1.00 | 0.00 | xxxx | 2230 |
| ATOM | 2231 | CG | ASP | A | 291 | 50.608 | 14.341 | 80.042 | 1.00 | 0.00 | xxxx | 2231 |
| ATOM | 2232 | OD1 | ASP | A | 291 | 51.635 | 13.723 | 79.686 | 1.00 | 0.00 | xxxx | 2232 |
| ATOM | 2233 | OD2 | ASP | A | 291 | 49.463 | 13.904 | 79.833 | 1.00 | 0.00 | xxxx | 2233 |
| ATOM | 2234 | N | GLY | A | 292 | 52.400 | 17.143 | 83.712 | 1.00 | 0.00 | xxxx | 2234 |
| ATOM | 2235 | CA | GLY | A | 292 | 52.765 | 18.411 | 84.313 | 1.00 | 0.00 | xxxx | 2235 |
| ATOM | 2236 | C | GLY | A | 292 | 51.636 | 19.388 | 84.564 | 1.00 | 0.00 | xxxx | 2236 |
| ATOM | 2237 | O | GLY | A | 292 | 51.868 | 20.414 | 85.213 | 1.00 | 0.00 | xxxx | 2237 |
| ATOM | 2238 | N | LYS | A | 293 | 50.420 | 19.117 | 84.082 | 1.00 | 0.00 | xxxx | 2238 |
| ATOM | 2239 | CA | LYS | A | 293 | 49.356 | 20.122 | 84.076 | 1.00 | 0.00 | xxxx | 2239 |
| ATOM | 2240 | C | LYS | A | 293 | 47.995 | 19.597 | 84.474 | 1.00 | 0.00 | xxxx | 2240 |
| ATOM | 2241 | O | LYS | A | 293 | 47.118 | 20.418 | 84.763 | 1.00 | 0.00 | xxxx | 2241 |
| ATOM | 2242 | CB | LYS | A | 293 | 49.212 | 20.750 | 82.682 | 1.00 | 0.00 | xxxx | 2242 |
| ATOM | 2243 | CG | LYS | A | 293 | 50.461 | 21.455 | 82.158 | 1.00 | 0.00 | xxxx | 2243 |
| ATOM | 2244 | CD | LYS | A | 293 | 50.271 | 21.857 | 80.705 | 1.00 | 0.00 | xxxx | 2244 |
| ATOM | 2245 | CE | LYS | A | 293 | 51.504 | 22.554 | 80.140 | 1.00 | 0.00 | xxxx | 2245 |
| ATOM | 2246 | NZ | LYS | A | 293 | 51.585 | 24.001 | 80.516 | 1.00 | 0.00 | xxxx | 2246 |
| ATOM | 2247 | N | TYR | A | 294 | 47.750 | 18.290 | 84.462 | 1.00 | 0.00 | xxxx | 2247 |
| ATOM | 2248 | CA | TYR | A | 294 | 46.396 | 17.766 | 84.562 | 1.00 | 0.00 | xxxx | 2248 |
| ATOM | 2249 | C | TYR | A | 294 | 46.234 | 16.963 | 85.842 | 1.00 | 0.00 | xxxx | 2249 |
| ATOM | 2250 | O | TYR | A | 294 | 47.120 | 16.196 | 86.223 | 1.00 | 0.00 | xxxx | 2250 |
| ATOM | 2251 | CB | TYR | A | 294 | 46.107 | 16.820 | 83.394 | 1.00 | 0.00 | xxxx | 2251 |
| ATOM | 2252 | CG | TYR | A | 294 | 46.133 | 17.445 | 82.024 | 1.00 | 0.00 | xxxx | 2252 |
| ATOM | 2253 | CD1 | TYR | A | 294 | 47.322 | 17.549 | 81.311 | 1.00 | 0.00 | xxxx | 2253 |
| ATOM | 2254 | CD2 | TYR | A | 294 | 44.970 | 17.922 | 81.426 | 1.00 | 0.00 | xxxx | 2254 |
| ATOM | 2255 | CE1 | TYR | A | 294 | 47.355 | 18.114 | 80.041 | 1.00 | 0.00 | xxxx | 2255 |
| ATOM | 2256 | CE2 | TYR | A | 294 | 44.989 | 18.493 | 80.157 | 1.00 | 0.00 | xxxx | 2256 |
| ATOM | 2257 | CZ | TYR | A | 294 | 46.178 | 18.580 | 79.466 | 1.00 | 0.00 | xxxx | 2257 |
| ATOM | 2258 | OH | TYR | A | 294 | 46.194 | 19.133 | 78.209 | 1.00 | 0.00 | xxxx | 2258 |
| ATOM | 2259 | N | VAL | A | 295 | 45.098 | 17.116 | 86.503 | 1.00 | 0.00 | xxxx | 2259 |
| ATOM | 2260 | CA | VAL | A | 295 | 44.716 | 16.234 | 87.600 | 1.00 | 0.00 | xxxx | 2260 |
| ATOM | 2261 | C | VAL | A | 295 | 43.472 | 15.475 | 87.175 | 1.00 | 0.00 | xxxx | 2261 |
| ATOM | 2262 | O | VAL | A | 295 | 42.414 | 16.086 | 86.951 | 1.00 | 0.00 | xxxx | 2262 |
| ATOM | 2263 | CB | VAL | A | 295 | 44.463 | 17.008 | 88.897 | 1.00 | 0.00 | xxxx | 2263 |
| ATOM | 2264 | CG1 | VAL | A | 295 | 43.877 | 16.067 | 89.959 | 1.00 | 0.00 | xxxx | 2264 |
| ATOM | 2265 | CG2 | VAL | A | 295 | 45.740 | 17.633 | 89.385 | 1.00 | 0.00 | xxxx | 2265 |
| ATOM | 2266 | N | TRP | A | 296 | 43.597 | 14.154 | 87.058 | 1.00 | 0.00 | xxxx | 2266 |
| ATOM | 2267 | CA | TRP | A | 296 | 42.499 | 13.286 | 86.651 | 1.00 | 0.00 | xxxx | 2267 |
| ATOM | 2268 | C | TRP | A | 296 | 41.963 | 12.562 | 87.875 | 1.00 | 0.00 | xxxx | 2268 |
| ATOM | 2269 | O | TRP | A | 296 | 42.720 | 11.879 | 88.579 | 1.00 | 0.00 | xxxx | 2269 |
| ATOM | 2270 | CB | TRP | A | 296 | 42.984 | 12.264 | 85.627 | 1.00 | 0.00 | xxxx | 2270 |
| ATOM | 2271 | CG | TRP | A | 296 | 43.564 | 12.875 | 84.394 | 1.00 | 0.00 | xxxx | 2271 |
| ATOM | 2272 | CD1 | TRP | A | 296 | 44.852 | 12.768 | 83.952 | 1.00 | 0.00 | xxxx | 2272 |
| ATOM | 2273 | CD2 | TRP | A | 296 | 42.868 | 13.664 | 83.428 | 1.00 | 0.00 | xxxx | 2273 |
| ATOM | 2274 | NE1 | TRP | A | 296 | 45.002 | 13.449 | 82.772 | 1.00 | 0.00 | xxxx | 2274 |
| ATOM | 2275 | CE2 | TRP | A | 296 | 43.799 | 14.014 | 82.430 | 1.00 | 0.00 | xxxx | 2275 |
| ATOM | 2276 | CE3 | TRP | A | 296 | 41.548 | 14.121 | 83.318 | 1.00 | 0.00 | xxxx | 2276 |
| ATOM | 2277 | CZ2 | TRP | A | 296 | 43.451 | 14.796 | 81.322 | 1.00 | 0.00 | xxxx | 2277 |
| ATOM | 2278 | CZ3 | TRP | A | 296 | 41.197 | 14.894 | 82.230 | 1.00 | 0.00 | xxxx | 2278 |
| ATOM | 2279 | CH2 | TRP | A | 296 | 42.143 | 15.230 | 81.242 | 1.00 | 0.00 | xxxx | 2279 |
| ATOM | 2280 | N | ILE | A | 297 | 40.669 | 12.705 | 88.116 | 1.00 | 0.00 | xxxx | 2280 |
| ATOM | 2281 | CA | ILE | A | 297 | 39.992 | 12.097 | 89.258 | 1.00 | 0.00 | xxxx | 2281 |
| ATOM | 2282 | C | ILE | A | 297 | 39.213 | 10.888 | 88.746 | 1.00 | 0.00 | xxxx | 2282 |
| ATOM | 2283 | O | ILE | A | 297 | 38.483 | 11.019 | 87.754 | 1.00 | 0.00 | xxxx | 2283 |
| ATOM | 2284 | CB | ILE | A | 297 | 39.048 | 13.123 | 89.911 | 1.00 | 0.00 | xxxx | 2284 |
| ATOM | 2285 | CG1 | ILE | A | 297 | 39.825 | 14.359 | 90.410 | 1.00 | 0.00 | xxxx | 2285 |
| ATOM | 2286 | CG2 | ILE | A | 297 | 38.234 | 12.494 | 91.020 | 1.00 | 0.00 | xxxx | 2286 |
| ATOM | 2287 | CD1 | ILE | A | 297 | 40.936 | 14.067 | 91.410 | 1.00 | 0.00 | xxxx | 2287 |
| ATOM | 2288 | N | PRO | A | 298 | 39.335 | 9.717 | 89.373 | 1.00 | 0.00 | xxxx | 2288 |
| ATOM | 2289 | CA | PRO | A | 298 | 38.673 | 8.511 | 88.848 | 1.00 | 0.00 | xxxx | 2289 |
| ATOM | 2290 | C | PRO | A | 298 | 37.153 | 8.600 | 88.852 | 1.00 | 0.00 | xxxx | 2290 |
| ATOM | 2291 | O | PRO | A | 298 | 36.541 | 9.254 | 89.701 | 1.00 | 0.00 | xxxx | 2291 |
| ATOM | 2292 | CB | PRO | A | 298 | 39.139 | 7.402 | 89.803 | 1.00 | 0.00 | xxxx | 2292 |
| ATOM | 2293 | CG | PRO | A | 298 | 40.414 | 7.921 | 90.386 | 1.00 | 0.00 | xxxx | 2293 |
| ATOM | 2294 | CD | PRO | A | 298 | 40.239 | 9.409 | 90.493 | 1.00 | 0.00 | xxxx | 2294 |
| ATOM | 2295 | N | TYR | A | 299 | 36.553 | 7.896 | 87.896 | 1.00 | 0.00 | xxxx | 2295 |
| ATOM | 2296 | CA | TYR | A | 299 | 35.127 | 7.600 | 87.859 | 1.00 | 0.00 | xxxx | 2296 |
| ATOM | 2297 | C | TYR | A | 299 | 34.886 | 6.243 | 88.512 | 1.00 | 0.00 | xxxx | 2297 |
| ATOM | 2298 | O | TYR | A | 299 | 35.783 | 5.406 | 88.587 | 1.00 | 0.00 | xxxx | 2298 |
| ATOM | 2299 | CB | TYR | A | 299 | 34.673 | 7.509 | 86.405 | 1.00 | 0.00 | xxxx | 2299 |
| ATOM | 2300 | CG | TYR | A | 299 | 34.448 | 8.814 | 85.684 | 1.00 | 0.00 | xxxx | 2300 |
| ATOM | 2301 | CD1 | TYR | A | 299 | 33.321 | 9.584 | 85.936 | 1.00 | 0.00 | xxxx | 2301 |
| ATOM | 2302 | CD2 | TYR | A | 299 | 35.339 | 9.257 | 84.695 | 1.00 | 0.00 | xxxx | 2302 |
| ATOM | 2303 | CE1 | TYR | A | 299 | 33.088 | 10.760 | 85.251 | 1.00 | 0.00 | xxxx | 2303 |
| ATOM | 2304 | CE2 | TYR | A | 299 | 35.117 | 10.428 | 84.012 | 1.00 | 0.00 | xxxx | 2304 |
| ATOM | 2305 | CZ | TYR | A | 299 | 33.974 | 11.176 | 84.286 | 1.00 | 0.00 | xxxx | 2305 |
| ATOM | 2306 | OH | TYR | A | 299 | 33.700 | 12.363 | 83.634 | 1.00 | 0.00 | xxxx | 2306 |
| ATOM | 2307 | N | LYS | A | 300 | 33.647 | 6.018 | 88.962 | 1.00 | 0.00 | xxxx | 2307 |
| ATOM | 2308 | CA | LYS | A | 300 | 33.281 | 4.772 | 89.636 | 1.00 | 0.00 | xxxx | 2308 |
| ATOM | 2309 | C | LYS | A | 300 | 31.892 | 4.324 | 89.199 | 1.00 | 0.00 | xxxx | 2309 |
| ATOM | 2310 | O | LYS | A | 300 | 30.955 | 5.125 | 89.201 | 1.00 | 0.00 | xxxx | 2310 |
| ATOM | 2311 | CB | LYS | A | 300 | 33.304 | 4.942 | 91.163 | 1.00 | 0.00 | xxxx | 2311 |
| ATOM | 2312 | CG | LYS | A | 300 | 32.995 | 3.686 | 91.943 | 1.00 | 0.00 | xxxx | 2312 |
| ATOM | 2313 | CD | LYS | A | 300 | 33.317 | 3.875 | 93.417 | 1.00 | 0.00 | xxxx | 2313 |
| ATOM | 2314 | CE | LYS | A | 300 | 33.211 | 2.565 | 94.182 | 1.00 | 0.00 | xxxx | 2314 |
| ATOM | 2315 | NZ | LYS | A | 300 | 33.711 | 2.702 | 95.580 | 1.00 | 0.00 | xxxx | 2315 |
| ATOM | 2316 | N | LYS | A | 301 | 31.750 | 3.047 | 88.847 | 1.00 | 0.00 | xxxx | 2316 |
| ATOM | 2317 | CA | LYS | A | 301 | 30.456 | 2.506 | 88.445 | 1.00 | 0.00 | xxxx | 2317 |
| ATOM | 2318 | C | LYS | A | 301 | 29.495 | 2.456 | 89.631 | 1.00 | 0.00 | xxxx | 2318 |
| ATOM | 2319 | O | LYS | A | 301 | 29.872 | 2.039 | 90.729 | 1.00 | 0.00 | xxxx | 2319 |
| ATOM | 2320 | CB | LYS | A | 301 | 30.668 | 1.089 | 87.913 | 1.00 | 0.00 | xxxx | 2320 |
| ATOM | 2321 | CG | LYS | A | 301 | 29.452 | 0.450 | 87.283 | 1.00 | 0.00 | xxxx | 2321 |
| ATOM | 2322 | CD | LYS | A | 301 | 29.731 | −1.015 | 86.917 | 1.00 | 0.00 | xxxx | 2322 |
| ATOM | 2323 | CE | LYS | A | 301 | 30.833 | −1.162 | 85.862 | 1.00 | 0.00 | xxxx | 2323 |
| ATOM | 2324 | NZ | LYS | A | 301 | 31.026 | −2.605 | 85.495 | 1.00 | 0.00 | xxxx | 2324 |
| ATOM | 2325 | N | ILE | A | 302 | 28.237 | 2.850 | 89.401 | 1.00 | 0.00 | xxxx | 2325 |
| ATOM | 2326 | CA | ILE | A | 302 | 27.199 | 2.842 | 90.435 | 1.00 | 0.00 | xxxx | 2326 |
| ATOM | 2327 | C | ILE | A | 302 | 25.950 | 2.144 | 89.895 | 1.00 | 0.00 | xxxx | 2327 |
| ATOM | 2328 | O | ILE | A | 302 | 25.369 | 2.584 | 88.892 | 1.00 | 0.00 | xxxx | 2328 |
| ATOM | 2329 | CB | ILE | A | 302 | 26.836 | 4.266 | 90.901 | 1.00 | 0.00 | xxxx | 2329 |
| ATOM | 2330 | CG1 | ILE | A | 302 | 28.054 | 5.047 | 91.407 | 1.00 | 0.00 | xxxx | 2330 |
| ATOM | 2331 | CG2 | ILE | A | 302 | 25.714 | 4.217 | 91.934 | 1.00 | 0.00 | xxxx | 2331 |
| ATOM | 2332 | CD1 | ILE | A | 302 | 28.689 | 4.485 | 92.677 | 1.00 | 0.00 | xxxx | 2332 |
| ATOM | 2333 | N | THR | A | 303 | 25.510 | 1.093 | 90.584 | 1.00 | 0.00 | xxxx | 2333 |
| ATOM | 2334 | CA | THR | A | 303 | 24.195 | 0.508 | 90.335 | 1.00 | 0.00 | xxxx | 2334 |
| ATOM | 2335 | C | THR | A | 303 | 23.474 | 0.302 | 91.663 | 1.00 | 0.00 | xxxx | 2335 |
| ATOM | 2336 | O | THR | A | 303 | 23.969 | 0.724 | 92.713 | 1.00 | 0.00 | xxxx | 2336 |
| ATOM | 2337 | CB | THR | A | 303 | 24.281 | −0.832 | 89.591 | 1.00 | 0.00 | xxxx | 2337 |
| ATOM | 2338 | OG1 | THR | A | 303 | 24.863 | −1.821 | 90.450 | 1.00 | 0.00 | xxxx | 2338 |
| ATOM | 2339 | CG2 | THR | A | 303 | 25.111 | −0.723 | 88.315 | 1.00 | 0.00 | xxxx | 2339 |
| ATOM | 2340 | O | LYS | A | 304 | 22.265 | −1.400 | 95.049 | 1.00 | 0.00 | xxxx | 2340 |
| ATOM | 2341 | N | LYS | A | 304 | 22.310 | −0.349 | 91.637 | 1.00 | 0.00 | xxxx | 2341 |
| ATOM | 2342 | CA | LYS | A | 304 | 21.616 | −0.622 | 92.889 | 1.00 | 0.00 | xxxx | 2342 |
| ATOM | 2343 | C | LYS | A | 304 | 22.469 | −1.461 | 93.833 | 1.00 | 0.00 | xxxx | 2343 |
| ATOM | 2344 | CB | LYS | A | 304 | 20.262 | −1.290 | 92.628 | 1.00 | 0.00 | xxxx | 2344 |
| ATOM | 2345 | CG | LYS | A | 304 | 20.350 | −2.695 | 92.059 | 1.00 | 0.00 | xxxx | 2345 |
| ATOM | 2346 | CD | LYS | A | 304 | 18.954 | −3.279 | 91.841 | 1.00 | 0.00 | xxxx | 2346 |
| ATOM | 2347 | CE | LYS | A | 304 | 19.020 | −4.700 | 91.304 | 1.00 | 0.00 | xxxx | 2347 |
| ATOM | 2348 | NZ | LYS | A | 304 | 17.663 | −5.253 | 91.028 | 1.00 | 0.00 | xxxx | 2348 |
| ATOM | 2349 | O | ASP | A | 305 | 25.678 | −2.904 | 96.040 | 1.00 | 0.00 | xxxx | 2349 |
| ATOM | 2350 | N | ASP | A | 305 | 23.438 | −2.214 | 93.303 | 1.00 | 0.00 | xxxx | 2350 |
| ATOM | 2351 | CA | ASP | A | 305 | 24.251 | −3.096 | 94.137 | 1.00 | 0.00 | xxxx | 2351 |
| ATOM | 2352 | C | ASP | A | 305 | 25.233 | −2.346 | 95.030 | 1.00 | 0.00 | xxxx | 2352 |
| ATOM | 2353 | CB | ASP | A | 305 | 25.015 | −4.096 | 93.267 | 1.00 | 0.00 | xxxx | 2353 |
| ATOM | 2354 | CG | ASP | A | 305 | 24.095 | −5.040 | 92.517 | 1.00 | 0.00 | xxxx | 2354 |
| ATOM | 2355 | OD1 | ASP | A | 305 | 22.980 | −5.309 | 93.014 | 1.00 | 0.00 | xxxx | 2355 |
| ATOM | 2356 | OD2 | ASP | A | 305 | 24.492 | −5.517 | 91.432 | 1.00 | 0.00 | xxxx | 2356 |
| ATOM | 2357 | O | ASN | A | 306 | 27.103 | 1.971 | 95.532 | 1.00 | 0.00 | xxxx | 2357 |
| ATOM | 2358 | N | ASN | A | 306 | 25.591 | −1.108 | 94.693 | 1.00 | 0.00 | xxxx | 2358 |
| ATOM | 2359 | CA | ASN | A | 306 | 26.605 | −0.378 | 95.451 | 1.00 | 0.00 | xxxx | 2359 |
| ATOM | 2360 | C | ASN | A | 306 | 26.239 | 1.091 | 95.577 | 1.00 | 0.00 | xxxx | 2360 |
| ATOM | 2361 | CB | ASN | A | 306 | 28.002 | −0.550 | 94.848 | 1.00 | 0.00 | xxxx | 2361 |
| ATOM | 2362 | CG | ASN | A | 306 | 28.157 | 0.154 | 93.506 | 1.00 | 0.00 | xxxx | 2362 |
| ATOM | 2363 | OD1 | ASN | A | 306 | 27.213 | 0.251 | 92.728 | 1.00 | 0.00 | xxxx | 2363 |
| ATOM | 2364 | ND2 | ASN | A | 306 | 29.350 | 0.652 | 93.237 | 1.00 | 0.00 | xxxx | 2364 |
| ATOM | 2365 | N | ILE | A | 307 | 24.949 | 1.374 | 95.760 | 1.00 | 0.00 | xxxx | 2365 |
| ATOM | 2366 | CA | ILE | A | 307 | 24.491 | 2.758 | 95.743 | 1.00 | 0.00 | xxxx | 2366 |
| ATOM | 2367 | C | ILE | A | 307 | 25.148 | 3.570 | 96.853 | 1.00 | 0.00 | xxxx | 2367 |
| ATOM | 2368 | O | ILE | A | 307 | 25.348 | 4.783 | 96.713 | 1.00 | 0.00 | xxxx | 2368 |
| ATOM | 2369 | CB | ILE | A | 307 | 22.947 | 2.803 | 95.773 | 1.00 | 0.00 | xxxx | 2369 |
| ATOM | 2370 | CG1 | ILE | A | 307 | 22.438 | 4.196 | 95.404 | 1.00 | 0.00 | xxxx | 2370 |
| ATOM | 2371 | CG2 | ILE | A | 307 | 22.418 | 2.342 | 97.124 | 1.00 | 0.00 | xxxx | 2371 |
| ATOM | 2372 | CD1 | ILE | A | 307 | 22.867 | 4.653 | 94.025 | 1.00 | 0.00 | xxxx | 2372 |
| ATOM | 2373 | O | SER | A | 308 | 28.067 | 5.083 | 99.317 | 1.00 | 0.00 | xxxx | 2373 |
| ATOM | 2374 | N | SER | A | 308 | 25.542 | 2.914 | 97.949 | 1.00 | 0.00 | xxxx | 2374 |
| ATOM | 2375 | CA | SER | A | 308 | 26.201 | 3.623 | 99.038 | 1.00 | 0.00 | xxxx | 2375 |
| ATOM | 2376 | C | SER | A | 308 | 27.576 | 4.164 | 98.655 | 1.00 | 0.00 | xxxx | 2376 |
| ATOM | 2377 | CB | SER | A | 308 | 26.294 | 2.732 | 100.281 | 1.00 | 0.00 | xxxx | 2377 |
| ATOM | 2378 | OG | SER | A | 308 | 27.025 | 1.548 | 100.014 | 1.00 | 0.00 | xxxx | 2378 |
| ATOM | 2379 | O | ASP | A | 309 | 30.354 | 6.303 | 96.505 | 1.00 | 0.00 | xxxx | 2379 |
| ATOM | 2380 | N | ASP | A | 309 | 28.208 | 3.628 | 97.602 | 1.00 | 0.00 | xxxx | 2380 |
| ATOM | 2381 | CA | ASP | A | 309 | 29.484 | 4.176 | 97.152 | 1.00 | 0.00 | xxxx | 2381 |
| ATOM | 2382 | C | ASP | A | 309 | 29.348 | 5.594 | 96.612 | 1.00 | 0.00 | xxxx | 2382 |
| ATOM | 2383 | CB | ASP | A | 309 | 30.119 | 3.293 | 96.076 | 1.00 | 0.00 | xxxx | 2383 |
| ATOM | 2384 | CG | ASP | A | 309 | 30.435 | 1.895 | 96.574 | 1.00 | 0.00 | xxxx | 2384 |
| ATOM | 2385 | OD1 | ASP | A | 309 | 30.291 | 1.646 | 97.790 | 1.00 | 0.00 | xxxx | 2385 |
| ATOM | 2386 | OD2 | ASP | A | 309 | 30.836 | 1.048 | 95.746 | 1.00 | 0.00 | xxxx | 2386 |
| ATOM | 2387 | O | ALA | A | 310 | 27.266 | 9.493 | 96.726 | 1.00 | 0.00 | xxxx | 2387 |
| ATOM | 2388 | N | ALA | A | 310 | 28.140 | 6.016 | 96.251 | 1.00 | 0.00 | xxxx | 2388 |
| ATOM | 2389 | CA | ALA | A | 310 | 27.910 | 7.386 | 95.816 | 1.00 | 0.00 | xxxx | 2389 |
| ATOM | 2390 | C | ALA | A | 310 | 27.485 | 8.298 | 96.957 | 1.00 | 0.00 | xxxx | 2390 |
| ATOM | 2391 | CB | ALA | A | 310 | 26.869 | 7.420 | 94.692 | 1.00 | 0.00 | xxxx | 2391 |
| ATOM | 2392 | O | ALA | A | 311 | 24.594 | 8.506 | 99.260 | 1.00 | 0.00 | xxxx | 2392 |
| ATOM | 2393 | N | ALA | A | 311 | 27.370 | 7.760 | 98.171 | 1.00 | 0.00 | xxxx | 2393 |
| ATOM | 2394 | CA | ALA | A | 311 | 26.990 | 8.515 | 99.366 | 1.00 | 0.00 | xxxx | 2394 |
| ATOM | 2395 | C | ALA | A | 311 | 25.625 | 9.178 | 99.219 | 1.00 | 0.00 | xxxx | 2395 |
| ATOM | 2396 | CB | ALA | A | 311 | 28.065 | 9.543 | 99.730 | 1.00 | 0.00 | xxxx | 2396 |
| HETATM | 2397 | CA | CA | B | 1 | 16.457 | −6.247 | 69.477 | 1.00 | 0.00 | xxxx | 2397 |
| HETATM | 2398 | K | K | C | 1 | 8.586 | −1.863 | 85.542 | 1.00 | 0.00 | xxxx | 2398 |
| HETATM | 2399 | C2 | GLC | D | 1 | 28.679 | 16.468 | 83.705 | 1.00 | 0.00 | xxxx | 2399 |
| HETATM | 2400 | C3 | GLC | D | 1 | 27.481 | 17.202 | 83.221 | 1.00 | 0.00 | xxxx | 2400 |
| HETATM | 2401 | C4 | GLC | D | 1 | 27.709 | 18.688 | 83.223 | 1.00 | 0.00 | xxxx | 2401 |
| HETATM | 2402 | C5 | GLC | D | 1 | 28.951 | 19.065 | 82.410 | 1.00 | 0.00 | xxxx | 2402 |
| HETATM | 2403 | C6 | GLC | D | 1 | 29.243 | 20.532 | 82.547 | 1.00 | 0.00 | xxxx | 2403 |
| HETATM | 2404 | C1 | GLC | D | 1 | 29.897 | 16.851 | 82.877 | 1.00 | 0.00 | xxxx | 2404 |
| HETATM | 2405 | O1 | GLC | D | 1 | 31.017 | 16.223 | 83.335 | 1.00 | 0.00 | xxxx | 2405 |
| HETATM | 2406 | O2 | GLC | D | 1 | 28.476 | 15.049 | 83.557 | 1.00 | 0.00 | xxxx | 2406 |
| HETATM | 2407 | O3 | GLC | D | 1 | 26.342 | 16.887 | 84.074 | 1.00 | 0.00 | xxxx | 2407 |
| HETATM | 2408 | O4 | GLC | D | 1 | 26.558 | 19.344 | 82.679 | 1.00 | 0.00 | xxxx | 2408 |
| HETATM | 2409 | O5 | GLC | D | 1 | 30.117 | 18.327 | 82.909 | 1.00 | 0.00 | xxxx | 2409 |
| HETATM | 2410 | O6 | GLC | D | 1 | 30.409 | 20.896 | 81.835 | 1.00 | 0.00 | xxxx | 2410 |
| HETATM | 2411 | C1 | EDO | E | 1 | 28.447 | 15.468 | 78.549 | 1.00 | 0.00 | xxxx | 2411 |
| HETATM | 2412 | O1 | EDO | E | 1 | 27.629 | 15.608 | 79.715 | 1.00 | 0.00 | xxxx | 2412 |
| HETATM | 2413 | C2 | EDO | E | 1 | 29.009 | 16.837 | 78.200 | 1.00 | 0.00 | xxxx | 2413 |
| HETATM | 2414 | O2 | EDO | E | 1 | 27.978 | 17.680 | 77.675 | 1.00 | 0.00 | xxxx | 2414 |
| HETATM | 2415 | C1 | EDO | E | 2 | 53.188 | 23.568 | 84.402 | 1.00 | 0.00 | xxxx | 2415 |
| HETATM | 2416 | O1 | EDO | E | 2 | 53.278 | 22.553 | 83.402 | 1.00 | 0.00 | xxxx | 2416 |
| HETATM | 2417 | C2 | EDO | E | 2 | 51.756 | 24.090 | 84.478 | 1.00 | 0.00 | xxxx | 2417 |
| HETATM | 2418 | O2 | EDO | E | 2 | 51.308 | 24.639 | 83.236 | 1.00 | 0.00 | xxxx | 2418 |
| HETATM | 2419 | C01 | ACR | H | 1 | 19.055 | 26.785 | 76.517 | 1.00 | 0.00 | xxxx | 2419 |
| HETATM | 2420 | N02 | ACR | H | 1 | 20.403 | 26.622 | 76.004 | 1.00 | 0.00 | xxxx | 2420 |
| HETATM | 2421 | C03 | ACR | H | 1 | 20.830 | 27.369 | 74.835 | 1.00 | 0.00 | xxxx | 2421 |
| HETATM | 2422 | C04 | ACR | H | 1 | 21.328 | 25.718 | 76.663 | 1.00 | 0.00 | xxxx | 2422 |
| HETATM | 2423 | C05 | ACR | H | 1 | 22.614 | 25.565 | 76.162 | 1.00 | 0.00 | xxxx | 2423 |
| HETATM | 2424 | C06 | ACR | H | 1 | 23.528 | 24.678 | 76.800 | 1.00 | 0.00 | xxxx | 2424 |
| HETATM | 2425 | C07 | ACR | H | 1 | 23.153 | 23.984 | 77.912 | 1.00 | 0.00 | xxxx | 2425 |
| HETATM | 2426 | C08 | ACR | H | 1 | 21.832 | 24.146 | 78.425 | 1.00 | 0.00 | xxxx | 2426 |
| HETATM | 2427 | C09 | ACR | H | 1 | 20.933 | 25.003 | 77.796 | 1.00 | 0.00 | xxxx | 2427 |
| HETATM | 2428 | C10 | ACR | H | 1 | 24.080 | 23.107 | 78.556 | 1.00 | 0.00 | xxxx | 2428 |
| HETATM | 2429 | C11 | ACR | H | 1 | 25.359 | 22.951 | 78.034 | 1.00 | 0.00 | xxxx | 2429 |
| HETATM | 2430 | C12 | ACR | H | 1 | 25.728 | 23.661 | 76.894 | 1.00 | 0.00 | xxxx | 2430 |
| HETATM | 2431 | C13 | ACR | H | 1 | 24.838 | 24.526 | 76.273 | 1.00 | 0.00 | xxxx | 2431 |
| HETATM | 2432 | C14 | ACR | H | 1 | 26.395 | 22.024 | 78.667 | 1.00 | 0.00 | xxxx | 2432 |
| HETATM | 2433 | O15 | ACR | H | 1 | 27.467 | 21.949 | 78.192 | 1.00 | 0.00 | xxxx | 2433 |
| HETATM | 2434 | C16 | ACR | H | 1 | 26.126 | 21.160 | 79.883 | 1.00 | 0.00 | xxxx | 2434 |
| HETATM | 2435 | C17 | ACR | H | 1 | 26.800 | 19.811 | 79.689 | 1.00 | 0.00 | xxxx | 2435 |
| HETATM | 2436 | O | HOH | S | 1 | 30.092 | 16.560 | 69.763 | 1.00 | 0.00 | xxxx | 2436 |
| HETATM | 2437 | O | HOH | S | 2 | 36.450 | 31.048 | 102.042 | 1.00 | 0.00 | xxxx | 2437 |
| HETATM | 2438 | O | HOH | S | 3 | 16.169 | 1.966 | 83.610 | 1.00 | 0.00 | xxxx | 2438 |
| HETATM | 2439 | O | HOH | S | 4 | 27.012 | −3.982 | 66.101 | 1.00 | 0.00 | xxxx | 2439 |
| HETATM | 2440 | O | HOH | S | 5 | 27.395 | 35.750 | 89.728 | 1.00 | 0.00 | xxxx | 2440 |
| HETATM | 2441 | O | HOH | S | 6 | 35.304 | 11.725 | 88.646 | 1.00 | 0.00 | xxxx | 2441 |
| HETATM | 2442 | O | HOH | S | 7 | 34.716 | 3.959 | 68.498 | 1.00 | 0.00 | xxxx | 2442 |
| HETATM | 2443 | O | HOH | S | 8 | 16.439 | 12.089 | 65.942 | 1.00 | 0.00 | xxxx | 2443 |
| HETATM | 2444 | O | HOH | S | 9 | 6.696 | 12.145 | 83.586 | 1.00 | 0.00 | xxxx | 2444 |
| HETATM | 2445 | O | HOH | S | 10 | 14.814 | −3.632 | 76.616 | 1.00 | 0.00 | xxxx | 2445 |
| HETATM | 2446 | O | HOH | S | 11 | 23.616 | 15.519 | 85.687 | 1.00 | 0.00 | xxxx | 2446 |
| HETATM | 2447 | O | HOH | S | 12 | 46.338 | 12.955 | 87.369 | 1.00 | 0.00 | xxxx | 2447 |
| HETATM | 2448 | O | HOH | S | 13 | 26.279 | 2.184 | 66.534 | 1.00 | 0.00 | xxxx | 2448 |
| HETATM | 2449 | O | HOH | S | 14 | 34.001 | 1.213 | 88.945 | 1.00 | 0.00 | xxxx | 2449 |
| HETATM | 2450 | O | HOH | S | 15 | 34.606 | 16.880 | 82.868 | 1.00 | 0.00 | xxxx | 2450 |
| HETATM | 2451 | O | HOH | S | 16 | 12.461 | 8.169 | 88.923 | 1.00 | 0.00 | xxxx | 2451 |
| HETATM | 2452 | O | HOH | S | 17 | 37.297 | 16.495 | 82.586 | 1.00 | 0.00 | xxxx | 2452 |
| HETATM | 2453 | O | HOH | S | 18 | 43.859 | 19.311 | 77.028 | 1.00 | 0.00 | xxxx | 2453 |
| HETATM | 2454 | O | HOH | S | 19 | 28.529 | −3.638 | 85.001 | 1.00 | 0.00 | xxxx | 2454 |
| HETATM | 2455 | O | HOH | S | 20 | 33.875 | 38.445 | 74.722 | 1.00 | 0.00 | xxxx | 2455 |
| HETATM | 2456 | O | HOH | S | 21 | 17.882 | 0.880 | 89.534 | 1.00 | 0.00 | xxxx | 2456 |
| HETATM | 2457 | O | HOH | S | 22 | 11.808 | 11.066 | 65.996 | 1.00 | 0.00 | xxxx | 2457 |
| HETATM | 2458 | O | HOH | S | 23 | 24.159 | −9.493 | 70.074 | 1.00 | 0.00 | xxxx | 2458 |
| HETATM | 2459 | O | HOH | S | 24 | 20.576 | −0.886 | 89.292 | 1.00 | 0.00 | xxxx | 2459 |
| HETATM | 2460 | O | HOH | S | 25 | 29.190 | 18.985 | 97.045 | 1.00 | 0.00 | xxxx | 2460 |
| HETATM | 2461 | O | HOH | S | 26 | 26.788 | 39.605 | 80.411 | 1.00 | 0.00 | xxxx | 2461 |
| HETATM | 2462 | O | HOH | S | 27 | 24.557 | 18.202 | 85.565 | 1.00 | 0.00 | xxxx | 2462 |
| HETATM | 2463 | O | HOH | S | 28 | 35.400 | 13.582 | 82.109 | 1.00 | 0.00 | xxxx | 2463 |
| HETATM | 2464 | O | HOH | S | 29 | 44.791 | 21.375 | 70.688 | 1.00 | 0.00 | xxxx | 2464 |
| HETATM | 2465 | O | HOH | S | 30 | 6.697 | 10.506 | 76.242 | 1.00 | 0.00 | xxxx | 2465 |
| HETATM | 2466 | O | HOH | S | 31 | 51.672 | 12.982 | 77.051 | 1.00 | 0.00 | xxxx | 2466 |
| HETATM | 2467 | O | HOH | S | 32 | 56.034 | 19.661 | 97.314 | 1.00 | 0.00 | xxxx | 2467 |
| HETATM | 2468 | O | HOH | S | 33 | 29.447 | 38.703 | 87.461 | 1.00 | 0.00 | xxxx | 2468 |
| HETATM | 2469 | O | HOH | S | 34 | 31.834 | 40.107 | 87.450 | 1.00 | 0.00 | xxxx | 2469 |
| HETATM | 2470 | O | HOH | S | 35 | 26.756 | 8.061 | 76.968 | 1.00 | 0.00 | xxxx | 2470 |
| HETATM | 2471 | O | HOH | S | 36 | 24.158 | −3.872 | 85.032 | 1.00 | 0.00 | xxxx | 2471 |
| HETATM | 2472 | O | HOH | S | 37 | 28.052 | −9.997 | 82.594 | 1.00 | 0.00 | xxxx | 2472 |
| HETATM | 2473 | O | HOH | S | 38 | 34.765 | −2.208 | 85.587 | 1.00 | 0.00 | xxxx | 2473 |
| HETATM | 2474 | O | HOH | S | 39 | 44.975 | 12.459 | 90.427 | 1.00 | 0.00 | xxxx | 2474 |
| HETATM | 2475 | O | HOH | S | 40 | 15.133 | −2.935 | 64.022 | 1.00 | 0.00 | xxxx | 2475 |
| HETATM | 2476 | O | HOH | S | 41 | 8.577 | 16.115 | 72.053 | 1.00 | 0.00 | xxxx | 2476 |
| HETATM | 2477 | O | HOH | S | 42 | 7.570 | 15.181 | 85.619 | 1.00 | 0.00 | xxxx | 2477 |
| HETATM | 2478 | O | HOH | S | 43 | 26.618 | 25.114 | 89.009 | 1.00 | 0.00 | xxxx | 2478 |
| HETATM | 2479 | O | HOH | S | 44 | 15.306 | 11.677 | 90.607 | 1.00 | 0.00 | xxxx | 2479 |
| HETATM | 2480 | O | HOH | S | 45 | 31.455 | 11.143 | 69.784 | 1.00 | 0.00 | xxxx | 2480 |
| HETATM | 2481 | O | HOH | S | 46 | 14.230 | −4.380 | 79.195 | 1.00 | 0.00 | xxxx | 2481 |
| HETATM | 2482 | O | HOH | S | 47 | 28.436 | 31.409 | 74.855 | 1.00 | 0.00 | xxxx | 2482 |
| HETATM | 2483 | O | HOH | S | 48 | 13.398 | −3.445 | 68.777 | 1.00 | 0.00 | xxxx | 2483 |
| HETATM | 2484 | O | HOH | S | 49 | 46.833 | 13.403 | 80.483 | 1.00 | 0.00 | xxxx | 2484 |
| HETATM | 2485 | O | HOH | S | 50 | 39.393 | 9.798 | 85.489 | 1.00 | 0.00 | xxxx | 2485 |
| HETATM | 2486 | O | HOH | S | 51 | 53.217 | 26.247 | 87.539 | 1.00 | 0.00 | xxxx | 2486 |
| HETATM | 2487 | O | HOH | S | 52 | 52.319 | 13.823 | 90.231 | 1.00 | 0.00 | xxxx | 2487 |
| HETATM | 2488 | O | HOH | S | 53 | 25.425 | −10.186 | 83.126 | 1.00 | 0.00 | xxxx | 2488 |
| HETATM | 2489 | O | HOH | S | 54 | 33.599 | −1.151 | 87.935 | 1.00 | 0.00 | xxxx | 2489 |
| HETATM | 2490 | O | HOH | S | 55 | 35.829 | −0.809 | 75.826 | 1.00 | 0.00 | xxxx | 2490 |
| HETATM | 2491 | O | HOH | S | 56 | 44.377 | 39.555 | 97.245 | 1.00 | 0.00 | xxxx | 2491 |
| HETATM | 2492 | O | HOH | S | 57 | 41.203 | 25.747 | 74.709 | 1.00 | 0.00 | xxxx | 2492 |
| HETATM | 2493 | O | HOH | S | 58 | 29.032 | 40.994 | 81.246 | 1.00 | 0.00 | xxxx | 2493 |
| HETATM | 2494 | O | HOH | S | 59 | 29.561 | 24.457 | 100.956 | 1.00 | 0.00 | xxxx | 2494 |
| HETATM | 2495 | O | HOH | S | 60 | 35.032 | 43.355 | 82.796 | 1.00 | 0.00 | xxxx | 2495 |
| HETATM | 2496 | O | HOH | S | 61 | 32.631 | −9.718 | 77.045 | 1.00 | 0.00 | xxxx | 2496 |
| HETATM | 2497 | O | HOH | S | 62 | 13.214 | 11.510 | 86.158 | 1.00 | 0.00 | xxxx | 2497 |
| HETATM | 2498 | O | HOH | S | 63 | 56.614 | 23.565 | 94.666 | 1.00 | 0.00 | xxxx | 2498 |
| HETATM | 2499 | O | HOH | S | 64 | 33.119 | −4.569 | 76.962 | 1.00 | 0.00 | xxxx | 2499 |
| HETATM | 2500 | O | HOH | S | 65 | 12.974 | 21.174 | 73.803 | 1.00 | 0.00 | xxxx | 2500 |
| HETATM | 2501 | O | HOH | S | 66 | 22.604 | 19.997 | 84.777 | 1.00 | 0.00 | xxxx | 2501 |
| HETATM | 2502 | O | HOH | S | 67 | 46.034 | 10.343 | 87.911 | 1.00 | 0.00 | xxxx | 2502 |
| HETATM | 2503 | O | HOH | S | 68 | 23.211 | 33.726 | 89.295 | 1.00 | 0.00 | xxxx | 2503 |
| HETATM | 2504 | O | HOH | S | 69 | 28.425 | 14.026 | 91.190 | 1.00 | 0.00 | xxxx | 2504 |
| HETATM | 2505 | O | HOH | S | 70 | 12.304 | 13.485 | 66.471 | 1.00 | 0.00 | xxxx | 2505 |
| HETATM | 2506 | O | HOH | S | 71 | 19.625 | 11.334 | 96.551 | 1.00 | 0.00 | xxxx | 2506 |
| HETATM | 2507 | O | HOH | S | 72 | 31.429 | −5.274 | 84.049 | 1.00 | 0.00 | xxxx | 2507 |
| HETATM | 2508 | O | HOH | S | 73 | 57.230 | 26.290 | 94.575 | 1.00 | 0.00 | xxxx | 2508 |
| HETATM | 2509 | O | HOH | S | 74 | 30.402 | 8.799 | 68.454 | 1.00 | 0.00 | xxxx | 2509 |
| HETATM | 2510 | O | HOH | S | 75 | 42.305 | 41.421 | 80.757 | 1.00 | 0.00 | xxxx | 2510 |
| HETATM | 2511 | O | HOH | S | 76 | 31.668 | −4.011 | 87.949 | 1.00 | 0.00 | xxxx | 2511 |
| HETATM | 2512 | O | HOH | S | 77 | 31.815 | 41.234 | 84.994 | 1.00 | 0.00 | xxxx | 2512 |
| HETATM | 2513 | O | HOH | S | 78 | 23.437 | 16.457 | 93.563 | 1.00 | 0.00 | xxxx | 2513 |
| HETATM | 2514 | O | HOH | S | 79 | 41.303 | 43.139 | 82.512 | 1.00 | 0.00 | xxxx | 2514 |
| HETATM | 2515 | O | HOH | S | 80 | 21.590 | 15.150 | 87.659 | 1.00 | 0.00 | xxxx | 2515 |
| HETATM | 2516 | O | HOH | S | 81 | 18.486 | 10.518 | 65.200 | 1.00 | 0.00 | xxxx | 2516 |
| HETATM | 2517 | O | HOH | S | 82 | 9.071 | −4.275 | 79.356 | 1.00 | 0.00 | xxxx | 2517 |
| HETATM | 2518 | O | HOH | S | 83 | 36.449 | 19.498 | 87.421 | 1.00 | 0.00 | xxxx | 2518 |
| HETATM | 2519 | O | HOH | S | 84 | 21.050 | −3.581 | 88.635 | 1.00 | 0.00 | xxxx | 2519 |
| HETATM | 2520 | O | HOH | S | 85 | 35.483 | 43.694 | 80.045 | 1.00 | 0.00 | xxxx | 2520 |
| HETATM | 2521 | O | HOH | S | 86 | 37.504 | 44.950 | 83.775 | 1.00 | 0.00 | xxxx | 2521 |
| HETATM | 2522 | O | HOH | S | 87 | 16.431 | 0.525 | 86.039 | 1.00 | 0.00 | xxxx | 2522 |
| HETATM | 2523 | O | HOH | S | 88 | 31.460 | 4.265 | 69.160 | 1.00 | 0.00 | xxxx | 2523 |
| HETATM | 2524 | O | HOH | S | 89 | 29.009 | −10.068 | 85.371 | 1.00 | 0.00 | xxxx | 2524 |
| HETATM | 2525 | O | HOH | S | 90 | 13.694 | 13.831 | 68.979 | 1.00 | 0.00 | xxxx | 2525 |
| HETATM | 2526 | O | HOH | S | 91 | 28.377 | 38.397 | 89.848 | 1.00 | 0.00 | xxxx | 2526 |
| HETATM | 2527 | O | HOH | S | 92 | 48.576 | 19.589 | 76.520 | 1.00 | 0.00 | xxxx | 2527 |
| HETATM | 2528 | O | HOH | S | 93 | 39.565 | 14.665 | 76.878 | 1.00 | 0.00 | xxxx | 2528 |
| HETATM | 2529 | O | HOH | S | 94 | 47.590 | 40.078 | 97.330 | 1.00 | 0.00 | xxxx | 2529 |
| HETATM | 2530 | O | HOH | S | 95 | 32.776 | 43.743 | 84.124 | 1.00 | 0.00 | xxxx | 2530 |
| HETATM | 2531 | O | HOH | S | 96 | 43.503 | 9.388 | 88.768 | 1.00 | 0.00 | xxxx | 2531 |
| HETATM | 2532 | O | HOH | S | 97 | 36.606 | 31.514 | 104.837 | 1.00 | 0.00 | xxxx | 2532 |
| HETATM | 2533 | O | HOH | S | 98 | 23.177 | 34.181 | 79.704 | 1.00 | 0.00 | xxxx | 2533 |
| HETATM | 2534 | O | HOH | S | 99 | 49.499 | 25.604 | 101.501 | 1.00 | 0.00 | xxxx | 2534 |
| HETATM | 2535 | O | HOH | S | 100 | 47.490 | 39.442 | 91.128 | 1.00 | 0.00 | xxxx | 2535 |
| HETATM | 2536 | O | HOH | S | 101 | 26.419 | −4.194 | 86.572 | 1.00 | 0.00 | xxxx | 2536 |
| HETATM | 2537 | O | HOH | S | 102 | 34.198 | 38.905 | 87.825 | 1.00 | 0.00 | xxxx | 2537 |
| HETATM | 2538 | O | HOH | S | 103 | 4.491 | 5.093 | 83.968 | 1.00 | 0.00 | xxxx | 2538 |
| HETATM | 2539 | O | HOH | S | 104 | 46.687 | 10.503 | 80.515 | 1.00 | 0.00 | xxxx | 2539 |
| HETATM | 2540 | O | HOH | S | 105 | 29.607 | −7.424 | 79.355 | 1.00 | 0.00 | xxxx | 2540 |
| HETATM | 2541 | O | HOH | S | 106 | 27.866 | 16.980 | 91.747 | 1.00 | 0.00 | xxxx | 2541 |
| HETATM | 2542 | O | HOH | S | 107 | 31.275 | 2.036 | 68.201 | 1.00 | 0.00 | xxxx | 2542 |
| HETATM | 2543 | O | HOH | S | 108 | 7.908 | 12.189 | 66.781 | 1.00 | 0.00 | xxxx | 2543 |
| HETATM | 2544 | O | HOH | S | 109 | 9.363 | −9.067 | 73.636 | 1.00 | 0.00 | xxxx | 2544 |
| HETATM | 2545 | O | HOH | S | 110 | 40.595 | 31.137 | 72.661 | 1.00 | 0.00 | xxxx | 2545 |
| HETATM | 2546 | O | HOH | S | 111 | 35.532 | 40.803 | 77.645 | 1.00 | 0.00 | xxxx | 2546 |
| HETATM | 2547 | O | HOH | S | 112 | 16.446 | 8.631 | 64.211 | 1.00 | 0.00 | xxxx | 2547 |
| HETATM | 2548 | O | HOH | S | 113 | 29.345 | 40.196 | 77.301 | 1.00 | 0.00 | xxxx | 2548 |
| HETATM | 2549 | O | HOH | S | 114 | 17.140 | −3.801 | 86.092 | 1.00 | 0.00 | xxxx | 2549 |
| HETATM | 2550 | O | HOH | S | 115 | 31.311 | −8.460 | 82.052 | 1.00 | 0.00 | xxxx | 2550 |
| HETATM | 2551 | O | HOH | S | 116 | 21.060 | −11.983 | 72.146 | 1.00 | 0.00 | xxxx | 2551 |
| HETATM | 2552 | O | HOH | S | 117 | 32.758 | 46.230 | 85.726 | 1.00 | 0.00 | xxxx | 2552 |
| HETATM | 2553 | O | HOH | S | 118 | 53.994 | 22.636 | 77.590 | 1.00 | 0.00 | xxxx | 2553 |
| HETATM | 2554 | O | HOH | S | 119 | 34.245 | 8.721 | 91.171 | 1.00 | 0.00 | xxxx | 2554 |
| HETATM | 2555 | O | HOH | S | 120 | 38.082 | 10.310 | 98.438 | 1.00 | 0.00 | xxxx | 2555 |
| HETATM | 2556 | O | HOH | S | 121 | 30.850 | 36.982 | 93.600 | 1.00 | 0.00 | xxxx | 2556 |
| HETATM | 2557 | O | HOH | S | 122 | 26.285 | 19.723 | 97.088 | 1.00 | 0.00 | xxxx | 2557 |
| HETATM | 2558 | O | HOH | S | 123 | 45.716 | 40.764 | 80.927 | 1.00 | 0.00 | xxxx | 2558 |
| HETATM | 2559 | O | HOH | S | 124 | 36.475 | 30.550 | 71.465 | 1.00 | 0.00 | xxxx | 2559 |
| HETATM | 2560 | O | HOH | S | 125 | 32.694 | 39.932 | 76.320 | 1.00 | 0.00 | xxxx | 2560 |
| HETATM | 2561 | O | HOH | S | 126 | 50.009 | 10.367 | 86.104 | 1.00 | 0.00 | xxxx | 2561 |
| HETATM | 2562 | O | HOH | S | 127 | 19.479 | −5.083 | 86.899 | 1.00 | 0.00 | xxxx | 2562 |
| HETATM | 2563 | O | HOH | S | 128 | 27.186 | 11.686 | 94.668 | 1.00 | 0.00 | xxxx | 2563 |
| HETATM | 2564 | O | HOH | S | 129 | 6.860 | 11.716 | 87.403 | 1.00 | 0.00 | xxxx | 2564 |
| HETATM | 2565 | O | HOH | S | 130 | 41.852 | 41.554 | 100.254 | 1.00 | 0.00 | xxxx | 2565 |
| HETATM | 2566 | O | HOH | S | 131 | 43.068 | 15.082 | 77.399 | 1.00 | 0.00 | xxxx | 2566 |
| HETATM | 2567 | O | HOH | S | 132 | 33.563 | −10.047 | 74.521 | 1.00 | 0.00 | xxxx | 2567 |
| HETATM | 2568 | O | HOH | S | 133 | 37.144 | 9.300 | 92.542 | 1.00 | 0.00 | xxxx | 2568 |
| HETATM | 2569 | O | HOH | S | 134 | 54.774 | 26.897 | 78.378 | 1.00 | 0.00 | xxxx | 2569 |
| HETATM | 2570 | O | HOH | S | 135 | 15.642 | 9.381 | 61.627 | 1.00 | 0.00 | xxxx | 2570 |
| HETATM | 2571 | O | HOH | S | 136 | 34.168 | 11.752 | 97.033 | 1.00 | 0.00 | xxxx | 2571 |
| HETATM | 2572 | O | HOH | S | 137 | 19.053 | −9.754 | 63.423 | 1.00 | 0.00 | xxxx | 2572 |
| HETATM | 2573 | O | HOH | S | 138 | 18.427 | 9.458 | 97.304 | 1.00 | 0.00 | xxxx | 2573 |
| HETATM | 2574 | O | HOH | S | 139 | 32.375 | 30.044 | 102.996 | 1.00 | 0.00 | xxxx | 2574 |
| HETATM | 2575 | O | HOH | S | 140 | 30.318 | −12.055 | 74.888 | 1.00 | 0.00 | xxxx | 2575 |
| HETATM | 2576 | O | HOH | S | 141 | 25.415 | 17.615 | 92.333 | 1.00 | 0.00 | xxxx | 2576 |
| HETATM | 2577 | O | HOH | S | 142 | 40.031 | 8.276 | 72.106 | 1.00 | 0.00 | xxxx | 2577 |
| HETATM | 2578 | O | HOH | S | 143 | 31.155 | −5.135 | 79.278 | 1.00 | 0.00 | xxxx | 2578 |
| HETATM | 2579 | O | HOH | S | 144 | 20.293 | −13.572 | 84.517 | 1.00 | 0.00 | xxxx | 2579 |
| HETATM | 2580 | O | HOH | S | 145 | 47.032 | 42.857 | 82.340 | 1.00 | 0.00 | xxxx | 2580 |
| HETATM | 2581 | O | HOH | S | 146 | 30.539 | 27.905 | 103.630 | 1.00 | 0.00 | xxxx | 2581 |
| HETATM | 2582 | O | HOH | S | 147 | 26.102 | −11.385 | 69.050 | 1.00 | 0.00 | xxxx | 2582 |
| HETATM | 2583 | O | HOH | S | 148 | 32.306 | 18.813 | 100.642 | 1.00 | 0.00 | xxxx | 2583 |
| HETATM | 2584 | O | HOH | S | 149 | 34.316 | 23.340 | 105.452 | 1.00 | 0.00 | xxxx | 2584 |
| HETATM | 2585 | O | HOH | S | 150 | 48.231 | 40.600 | 93.204 | 1.00 | 0.00 | xxxx | 2585 |
| HETATM | 2586 | O | HOH | S | 151 | 26.105 | 32.866 | 74.276 | 1.00 | 0.00 | xxxx | 2586 |
| HETATM | 2587 | O | HOH | S | 152 | 26.276 | −7.076 | 87.052 | 1.00 | 0.00 | xxxx | 2587 |
| HETATM | 2588 | O | HOH | S | 153 | 28.787 | 27.403 | 97.004 | 1.00 | 0.00 | xxxx | 2588 |
| HETATM | 2589 | O | HOH | S | 154 | 17.129 | −12.891 | 74.110 | 1.00 | 0.00 | xxxx | 2589 |
| HETATM | 2590 | O | HOH | S | 155 | 9.512 | 14.016 | 65.802 | 1.00 | 0.00 | xxxx | 2590 |
| HETATM | 2591 | O | HOH | S | 156 | 21.215 | 17.960 | 93.737 | 1.00 | 0.00 | xxxx | 2591 |
| HETATM | 2592 | O | HOH | S | 157 | 41.716 | 8.612 | 86.096 | 1.00 | 0.00 | xxxx | 2592 |
| HETATM | 2593 | O | HOH | S | 158 | 29.408 | −12.119 | 70.047 | 1.00 | 0.00 | xxxx | 2593 |
| HETATM | 2594 | O | HOH | S | 159 | 25.011 | −12.819 | 82.517 | 1.00 | 0.00 | xxxx | 2594 |
| HETATM | 2595 | O | HOH | S | 160 | 32.663 | −5.811 | 81.679 | 1.00 | 0.00 | xxxx | 2595 |
| HETATM | 2596 | O | HOH | S | 161 | 24.880 | 19.576 | 95.065 | 1.00 | 0.00 | xxxx | 2596 |
| HETATM | 2597 | O | HOH | S | 162 | 44.355 | 29.891 | 71.184 | 1.00 | 0.00 | xxxx | 2597 |
| HETATM | 2598 | O | HOH | S | 163 | 34.518 | 29.416 | 101.548 | 1.00 | 0.00 | xxxx | 2598 |
| HETATM | 2599 | O | HOH | S | 164 | 35.180 | 32.267 | 100.121 | 1.00 | 0.00 | xxxx | 2599 |
| HETATM | 2600 | O | HOH | S | 165 | 23.399 | −10.513 | 72.364 | 1.00 | 0.00 | xxxx | 2600 |
| HETATM | 2601 | O | HOH | S | 166 | 16.052 | 14.146 | 67.637 | 1.00 | 0.00 | xxxx | 2601 |
| HETATM | 2602 | O | HOH | S | 167 | 14.427 | −6.559 | 82.883 | 1.00 | 0.00 | xxxx | 2602 |
| HETATM | 2603 | O | HOH | S | 168 | 13.244 | −2.741 | 65.936 | 1.00 | 0.00 | xxxx | 2603 |
| HETATM | 2604 | O | HOH | S | 169 | 7.373 | 2.815 | 64.826 | 1.00 | 0.00 | xxxx | 2604 |
| HETATM | 2605 | O | HOH | S | 170 | 19.049 | −9.891 | 75.701 | 1.00 | 0.00 | xxxx | 2605 |
| HETATM | 2606 | O | HOH | S | 171 | 45.770 | 25.436 | 70.089 | 1.00 | 0.00 | xxxx | 2606 |
| HETATM | 2607 | O | HOH | S | 172 | 14.520 | 22.711 | 75.198 | 1.00 | 0.00 | xxxx | 2607 |
| HETATM | 2608 | O | HOH | S | 173 | 11.837 | −6.376 | 82.392 | 1.00 | 0.00 | xxxx | 2608 |
| HETATM | 2609 | O | HOH | S | 174 | 42.682 | 22.064 | 101.116 | 1.00 | 0.00 | xxxx | 2609 |
| HETATM | 2610 | O | HOH | S | 175 | 11.747 | −5.386 | 79.675 | 1.00 | 0.00 | xxxx | 2610 |
| HETATM | 2611 | O | HOH | S | 176 | 51.980 | 18.978 | 79.010 | 1.00 | 0.00 | xxxx | 2611 |
| HETATM | 2612 | O | HOH | S | 177 | 28.850 | −7.885 | 86.063 | 1.00 | 0.00 | xxxx | 2612 |
| HETATM | 2613 | O | HOH | S | 178 | 15.864 | −12.993 | 83.108 | 1.00 | 0.00 | xxxx | 2613 |
| HETATM | 2614 | O | HOH | S | 179 | 12.312 | −0.800 | 87.994 | 1.00 | 0.00 | xxxx | 2614 |
| HETATM | 2615 | O | HOH | S | 180 | 22.300 | 7.722 | 64.043 | 1.00 | 0.00 | xxxx | 2615 |
| HETATM | 2616 | O | HOH | S | 181 | 15.971 | 21.384 | 82.137 | 1.00 | 0.00 | xxxx | 2616 |
| HETATM | 2617 | O | HOH | S | 182 | 30.811 | 25.553 | 103.189 | 1.00 | 0.00 | xxxx | 2617 |
| HETATM | 2618 | O | HOH | S | 183 | 29.318 | 13.972 | 70.591 | 1.00 | 0.00 | xxxx | 2618 |
| HETATM | 2619 | O | HOH | S | 184 | 27.674 | 25.600 | 77.740 | 1.00 | 0.00 | xxxx | 2619 |
| HETATM | 2620 | O | HOH | S | 185 | 32.834 | 23.568 | 75.565 | 1.00 | 0.00 | xxxx | 2620 |
| HETATM | 2621 | O | HOH | S | 186 | 15.402 | −10.518 | 83.634 | 1.00 | 0.00 | xxxx | 2621 |
| HETATM | 2622 | O | HOH | S | 187 | 29.808 | 39.756 | 83.574 | 1.00 | 0.00 | xxxx | 2622 |
| HETATM | 2623 | O | HOH | S | 188 | 33.887 | 31.270 | 104.772 | 1.00 | 0.00 | xxxx | 2623 |
| HETATM | 2624 | O | HOH | S | 189 | 20.488 | −11.771 | 76.677 | 1.00 | 0.00 | xxxx | 2624 |
| HETATM | 2625 | O | HOH | S | 190 | 12.518 | 16.212 | 68.881 | 1.00 | 0.00 | xxxx | 2625 |
| HETATM | 2626 | O | HOH | S | 191 | 54.565 | 27.676 | 89.397 | 1.00 | 0.00 | xxxx | 2626 |
| HETATM | 2627 | O | HOH | S | 192 | 53.012 | 16.808 | 74.423 | 1.00 | 0.00 | xxxx | 2627 |
| HETATM | 2628 | O | HOH | S | 193 | 49.804 | 36.751 | 76.677 | 1.00 | 0.00 | xxxx | 2628 |
| HETATM | 2629 | O | HOH | S | 194 | 49.635 | 35.394 | 91.979 | 1.00 | 0.00 | xxxx | 2629 |
| HETATM | 2630 | O | HOH | S | 195 | 13.533 | 9.420 | 90.707 | 1.00 | 0.00 | xxxx | 2630 |
| HETATM | 2631 | O | HOH | S | 196 | 36.374 | −7.847 | 71.968 | 1.00 | 0.00 | xxxx | 2631 |
| HETATM | 2632 | O | HOH | S | 197 | 53.604 | 36.390 | 82.878 | 1.00 | 0.00 | xxxx | 2632 |
| HETATM | 2633 | O | HOH | S | 198 | 51.181 | 15.057 | 75.617 | 1.00 | 0.00 | xxxx | 2633 |
| HETATM | 2634 | O | HOH | S | 199 | 28.232 | 28.892 | 94.774 | 1.00 | 0.00 | xxxx | 2634 |
| HETATM | 2635 | O | HOH | S | 200 | 31.413 | 23.690 | 105.190 | 1.00 | 0.00 | xxxx | 2635 |
| HETATM | 2636 | O | HOH | S | 201 | 35.263 | −5.032 | 85.936 | 1.00 | 0.00 | xxxx | 2636 |
| HETATM | 2637 | O | HOH | S | 202 | 30.282 | 32.380 | 96.955 | 1.00 | 0.00 | xxxx | 2637 |
| HETATM | 2638 | O | HOH | S | 203 | 28.203 | 31.685 | 95.320 | 1.00 | 0.00 | xxxx | 2638 |
| HETATM | 2639 | O | HOH | S | 204 | 49.772 | 37.811 | 90.376 | 1.00 | 0.00 | xxxx | 2639 |
| HETATM | 2640 | O | HOH | S | 205 | 42.346 | 31.385 | 70.795 | 1.00 | 0.00 | xxxx | 2640 |
| HETATM | 2641 | O | HOH | S | 206 | 31.448 | 13.764 | 94.887 | 1.00 | 0.00 | xxxx | 2641 |
| HETATM | 2642 | O | HOH | S | 207 | 45.518 | 14.293 | 78.127 | 1.00 | 0.00 | xxxx | 2642 |
| HETATM | 2643 | O | HOH | S | 208 | 28.442 | 5.875 | 69.185 | 1.00 | 0.00 | xxxx | 2643 |
| HETATM | 2644 | O | HOH | S | 209 | 49.554 | 35.069 | 106.432 | 1.00 | 0.00 | xxxx | 2644 |
| HETATM | 2645 | O | HOH | S | 210 | 27.200 | 6.622 | 67.192 | 1.00 | 0.00 | xxxx | 2645 |
| HETATM | 2646 | O | HOH | S | 211 | 31.168 | 33.125 | 101.975 | 1.00 | 0.00 | xxxx | 2646 |
| HETATM | 2647 | O | HOH | S | 212 | 26.450 | 36.711 | 86.006 | 1.00 | 0.00 | xxxx | 2647 |
| HETATM | 2648 | O | HOH | S | 213 | 55.824 | 32.193 | 83.580 | 1.00 | 0.00 | xxxx | 2648 |
| HETATM | 2649 | O | HOH | S | 214 | 13.304 | 10.253 | 88.335 | 1.00 | 0.00 | xxxx | 2649 |
| HETATM | 2650 | O | HOH | S | 215 | 29.506 | 13.799 | 93.673 | 1.00 | 0.00 | xxxx | 2650 |
| HETATM | 2651 | O | HOH | S | 216 | 20.507 | 9.464 | 64.402 | 1.00 | 0.00 | xxxx | 2651 |
| HETATM | 2652 | O | HOH | S | 217 | 14.748 | 13.431 | 92.671 | 1.00 | 0.00 | xxxx | 2652 |
| HETATM | 2653 | O | HOH | S | 218 | 26.847 | 39.185 | 77.674 | 1.00 | 0.00 | xxxx | 2653 |
| HETATM | 2654 | O | HOH | S | 219 | 10.406 | 16.438 | 66.823 | 1.00 | 0.00 | xxxx | 2654 |
| HETATM | 2655 | O | HOH | S | 220 | 2.716 | 4.132 | 75.046 | 1.00 | 0.00 | xxxx | 2655 |
| HETATM | 2656 | O | HOH | S | 221 | 32.567 | −2.540 | 89.806 | 1.00 | 0.00 | xxxx | 2656 |
| HETATM | 2657 | O | HOH | S | 222 | 48.057 | 8.630 | 88.418 | 1.00 | 0.00 | xxxx | 2657 |
| HETATM | 2658 | O | HOH | S | 223 | 38.623 | 36.281 | 77.233 | 1.00 | 0.00 | xxxx | 2658 |
| HETATM | 2659 | O | HOH | S | 224 | 5.367 | 0.438 | 69.681 | 1.00 | 0.00 | xxxx | 2659 |
| HETATM | 2660 | O | HOH | S | 225 | 55.509 | 29.545 | 79.721 | 1.00 | 0.00 | xxxx | 2660 |
| HETATM | 2661 | O | HOH | S | 226 | 45.784 | 11.649 | 77.043 | 1.00 | 0.00 | xxxx | 2661 |
| HETATM | 2662 | O | HOH | S | 227 | 46.921 | 22.993 | 98.717 | 1.00 | 0.00 | xxxx | 2662 |
| HETATM | 2663 | O | HOH | S | 228 | 14.908 | −6.015 | 85.254 | 1.00 | 0.00 | xxxx | 2663 |
| HETATM | 2664 | O | HOH | S | 229 | 41.066 | 29.306 | 109.579 | 1.00 | 0.00 | xxxx | 2664 |
| HETATM | 2665 | O | HOH | S | 230 | 22.133 | 30.392 | 83.355 | 1.00 | 0.00 | xxxx | 2665 |
| HETATM | 2666 | O | HOH | S | 231 | 41.120 | 9.979 | 74.044 | 1.00 | 0.00 | xxxx | 2666 |
| HETATM | 2667 | O | HOH | S | 232 | 51.131 | 37.451 | 106.185 | 1.00 | 0.00 | xxxx | 2667 |
| HETATM | 2668 | O | HOH | S | 233 | 55.796 | 23.522 | 81.726 | 1.00 | 0.00 | xxxx | 2668 |
| HETATM | 2669 | O | HOH | S | 234 | 30.767 | 13.535 | 69.149 | 1.00 | 0.00 | xxxx | 2669 |
| HETATM | 2670 | O | HOH | S | 235 | 30.378 | −13.413 | 72.516 | 1.00 | 0.00 | xxxx | 2670 |
| HETATM | 2671 | O | HOH | S | 236 | 32.947 | 5.925 | 96.446 | 1.00 | 0.00 | xxxx | 2671 |
| HETATM | 2672 | O | HOH | S | 237 | 25.500 | 16.096 | 79.243 | 1.00 | 0.00 | xxxx | 2672 |
| HETATM | 2673 | O | HOH | S | 238 | 26.802 | 29.069 | 75.273 | 1.00 | 0.00 | xxxx | 2673 |
| HETATM | 2674 | O | HOH | S | 239 | 14.192 | 17.963 | 67.834 | 1.00 | 0.00 | xxxx | 2674 |
| HETATM | 2675 | O | HOH | S | 240 | 5.905 | 10.203 | 73.585 | 1.00 | 0.00 | xxxx | 2675 |
| HETATM | 2676 | O | HOH | S | 241 | 4.513 | 7.800 | 73.072 | 1.00 | 0.00 | xxxx | 2676 |
| HETATM | 2677 | O | HOH | S | 242 | 43.660 | 44.657 | 83.268 | 1.00 | 0.00 | xxxx | 2677 |
| HETATM | 2678 | O | HOH | S | 243 | 22.969 | 21.825 | 70.556 | 1.00 | 0.00 | xxxx | 2678 |
| HETATM | 2679 | O | HOH | S | 244 | 22.901 | −13.995 | 84.304 | 1.00 | 0.00 | xxxx | 2679 |
| HETATM | 2680 | O | HOH | S | 245 | 14.508 | 14.371 | 65.000 | 1.00 | 0.00 | xxxx | 2680 |
| HETATM | 2681 | O | HOH | S | 246 | 9.488 | 16.688 | 86.964 | 1.00 | 0.00 | xxxx | 2681 |
| HETATM | 2682 | O | HOH | S | 247 | 54.267 | 24.642 | 79.573 | 1.00 | 0.00 | xxxx | 2682 |
| HETATM | 2683 | O | HOH | S | 248 | 38.086 | 13.849 | 99.533 | 1.00 | 0.00 | xxxx | 2683 |
| HETATM | 2684 | O | HOH | S | 249 | 30.344 | 25.159 | 107.966 | 1.00 | 0.00 | xxxx | 2684 |
| HETATM | 2685 | O | HOH | S | 250 | 27.639 | −1.869 | 64.685 | 1.00 | 0.00 | xxxx | 2685 |
| HETATM | 2686 | O | HOH | S | 251 | 37.885 | 24.209 | 105.067 | 1.00 | 0.00 | xxxx | 2686 |
| HETATM | 2687 | O | HOH | S | 252 | 24.776 | −0.007 | 98.826 | 1.00 | 0.00 | xxxx | 2687 |
| HETATM | 2688 | O | HOH | S | 253 | 30.015 | 19.826 | 99.611 | 1.00 | 0.00 | xxxx | 2688 |
| HETATM | 2689 | O | HOH | S | 254 | 23.631 | −4.295 | 63.805 | 1.00 | 0.00 | xxxx | 2689 |
| HETATM | 2690 | O | HOH | S | 255 | 18.259 | −12.433 | 71.943 | 1.00 | 0.00 | xxxx | 2690 |
| HETATM | 2691 | O | HOH | S | 256 | 14.315 | 10.556 | 64.808 | 1.00 | 0.00 | xxxx | 2691 |
| HETATM | 2692 | O | HOH | S | 257 | 41.561 | 5.948 | 79.800 | 1.00 | 0.00 | xxxx | 2692 |
| HETATM | 2693 | O | HOH | S | 258 | 54.871 | 21.422 | 81.888 | 1.00 | 0.00 | xxxx | 2693 |
| HETATM | 2694 | O | HOH | S | 259 | 10.817 | −9.778 | 75.712 | 1.00 | 0.00 | xxxx | 2694 |
| HETATM | 2695 | O | HOH | S | 260 | 47.176 | 25.537 | 68.505 | 1.00 | 0.00 | xxxx | 2695 |
| HETATM | 2696 | O | HOH | S | 261 | 25.754 | −0.489 | 64.311 | 1.00 | 0.00 | xxxx | 2696 |
| HETATM | 2697 | O | HOH | S | 262 | 20.717 | 32.971 | 83.280 | 1.00 | 0.00 | xxxx | 2697 |
| HETATM | 2698 | O | HOH | S | 263 | 21.527 | 20.805 | 93.076 | 1.00 | 0.00 | xxxx | 2698 |
| HETATM | 2699 | O | HOH | S | 264 | 35.227 | 33.913 | 73.496 | 1.00 | 0.00 | xxxx | 2699 |
| HETATM | 2700 | O | HOH | S | 265 | 9.419 | 3.884 | 91.548 | 1.00 | 0.00 | xxxx | 2700 |
| HETATM | 2701 | O | HOH | S | 266 | 30.772 | 42.236 | 89.132 | 1.00 | 0.00 | xxxx | 2701 |
| HETATM | 2702 | O | HOH | S | 267 | 14.889 | 11.697 | 60.585 | 1.00 | 0.00 | xxxx | 2702 |
| HETATM | 2703 | O | HOH | S | 268 | 16.782 | −10.334 | 62.350 | 1.00 | 0.00 | xxxx | 2703 |
| HETATM | 2704 | O | HOH | S | 269 | 10.923 | −4.229 | 70.024 | 1.00 | 0.00 | xxxx | 2704 |
| HETATM | 2705 | O | HOH | S | 270 | 18.813 | 20.066 | 83.551 | 1.00 | 0.00 | xxxx | 2705 |
| HETATM | 2706 | O | HOH | S | 271 | 30.048 | 2.118 | 100.339 | 1.00 | 0.00 | xxxx | 2706 |
| HETATM | 2707 | O | HOH | S | 272 | 8.909 | −5.707 | 70.349 | 1.00 | 0.00 | xxxx | 2707 |
| HETATM | 2708 | O | HOH | S | 273 | 38.277 | 9.852 | 70.341 | 1.00 | 0.00 | xxxx | 2708 |
| HETATM | 2709 | O | HOH | S | 274 | 17.404 | 17.026 | 87.678 | 1.00 | 0.00 | xxxx | 2709 |
| HETATM | 2710 | O | HOH | S | 275 | 2.083 | 0.874 | 81.599 | 1.00 | 0.00 | xxxx | 2710 |
| HETATM | 2711 | O | HOH | S | 276 | 40.573 | 25.786 | 107.080 | 1.00 | 0.00 | xxxx | 2711 |
| HETATM | 2712 | O | HOH | S | 277 | 47.238 | 18.686 | 100.507 | 1.00 | 0.00 | xxxx | 2712 |
| HETATM | 2713 | O | HOH | S | 278 | 45.356 | 30.895 | 104.313 | 1.00 | 0.00 | xxxx | 2713 |
| HETATM | 2714 | O | HOH | S | 279 | 37.886 | 0.890 | 74.951 | 1.00 | 0.00 | xxxx | 2714 |
| HETATM | 2715 | O | HOH | S | 280 | 44.882 | 32.449 | 71.587 | 1.00 | 0.00 | xxxx | 2715 |
| HETATM | 2716 | O | HOH | S | 281 | 45.071 | 19.230 | 101.060 | 1.00 | 0.00 | xxxx | 2716 |
| HETATM | 2717 | O | HOH | S | 282 | 53.338 | 21.767 | 88.706 | 1.00 | 0.00 | xxxx | 2717 |
| HETATM | 2718 | O | HOH | S | 283 | 38.872 | 12.899 | 78.989 | 1.00 | 0.00 | xxxx | 2718 |
| HETATM | 2719 | O | HOH | S | 284 | 50.214 | 34.572 | 75.391 | 1.00 | 0.00 | xxxx | 2719 |
| HETATM | 2720 | O | HOH | S | 285 | 9.787 | 22.391 | 75.366 | 1.00 | 0.00 | xxxx | 2720 |
| HETATM | 2721 | O | HOH | S | 286 | 25.330 | 34.970 | 91.174 | 1.00 | 0.00 | xxxx | 2721 |
| HETATM | 2722 | O | HOH | S | 287 | 19.025 | 22.704 | 75.489 | 1.00 | 0.00 | xxxx | 2722 |
| HETATM | 2723 | O | HOH | S | 288 | 6.732 | 14.329 | 70.989 | 1.00 | 0.00 | xxxx | 2723 |
| HETATM | 2724 | O | HOH | S | 289 | 28.593 | 23.713 | 71.566 | 1.00 | 0.00 | xxxx | 2724 |
| HETATM | 2725 | O | HOH | S | 290 | 48.417 | 10.687 | 76.682 | 1.00 | 0.00 | xxxx | 2725 |
| HETATM | 2726 | O | HOH | S | 291 | 9.233 | 23.335 | 72.944 | 1.00 | 0.00 | xxxx | 2726 |
| HETATM | 2727 | O | HOH | S | 292 | 19.769 | −1.136 | 96.415 | 1.00 | 0.00 | xxxx | 2727 |
| HETATM | 2728 | O | HOH | S | 293 | 15.597 | 23.489 | 79.366 | 1.00 | 0.00 | xxxx | 2728 |
| HETATM | 2729 | O | HOH | S | 294 | 48.418 | 12.805 | 75.957 | 1.00 | 0.00 | xxxx | 2729 |
| HETATM | 2730 | O | HOH | S | 295 | 24.690 | 36.388 | 80.735 | 1.00 | 0.00 | xxxx | 2730 |
| HETATM | 2731 | O | HOH | S | 296 | 3.277 | 6.734 | 67.960 | 1.00 | 0.00 | xxxx | 2731 |
| HETATM | 2732 | O | HOH | S | 297 | 33.665 | 24.109 | 72.624 | 1.00 | 0.00 | xxxx | 2732 |
| HETATM | 2733 | O | HOH | S | 298 | 22.977 | 21.117 | 82.694 | 1.00 | 0.00 | xxxx | 2733 |
| HETATM | 2734 | O | HOH | S | 299 | 29.752 | −7.071 | 68.645 | 1.00 | 0.00 | xxxx | 2734 |
| HETATM | 2735 | O | HOH | S | 300 | 6.795 | 14.005 | 67.913 | 1.00 | 0.00 | xxxx | 2735 |
| HETATM | 2736 | O | HOH | S | 301 | 28.876 | 7.221 | 65.323 | 1.00 | 0.00 | xxxx | 2736 |
| HETATM | 2737 | O | HOH | S | 302 | 54.121 | 19.945 | 78.093 | 1.00 | 0.00 | xxxx | 2737 |
| HETATM | 2738 | O | HOH | S | 303 | 28.461 | −1.344 | 90.238 | 1.00 | 0.00 | xxxx | 2738 |
| HETATM | 2739 | O | HOH | S | 304 | 32.077 | 0.296 | 91.320 | 1.00 | 0.00 | xxxx | 2739 |
| HETATM | 2740 | O | HOH | S | 305 | 52.762 | 33.840 | 89.496 | 1.00 | 0.00 | xxxx | 2740 |
| HETATM | 2741 | O | HOH | S | 306 | 33.055 | 24.497 | 109.572 | 1.00 | 0.00 | xxxx | 2741 |
| HETATM | 2742 | O | HOH | S | 307 | 17.403 | 7.927 | 60.363 | 1.00 | 0.00 | xxxx | 2742 |
| HETATM | 2743 | O | HOH | S | 308 | 43.466 | 33.878 | 102.677 | 1.00 | 0.00 | xxxx | 2743 |
| HETATM | 2744 | O | HOH | S | 309 | 12.277 | 17.804 | 70.661 | 1.00 | 0.00 | xxxx | 2744 |
| HETATM | 2745 | O | HOH | S | 310 | 11.041 | 11.796 | 89.974 | 1.00 | 0.00 | xxxx | 2745 |
| HETATM | 2746 | O | HOH | S | 311 | 23.651 | 14.294 | 65.562 | 1.00 | 0.00 | xxxx | 2746 |
| HETATM | 2747 | O | HOH | S | 312 | 42.026 | 45.298 | 94.042 | 1.00 | 0.00 | xxxx | 2747 |
| HETATM | 2748 | O | HOH | S | 313 | 22.517 | 16.293 | 65.454 | 1.00 | 0.00 | xxxx | 2748 |
| HETATM | 2749 | O | HOH | S | 314 | 22.627 | 12.480 | 64.518 | 1.00 | 0.00 | xxxx | 2749 |
| HETATM | 2750 | O | HOH | S | 315 | 2.884 | 8.870 | 71.069 | 1.00 | 0.00 | xxxx | 2750 |
| HETATM | 2751 | O | HOH | S | 316 | 33.745 | 16.834 | 85.547 | 1.00 | 0.00 | xxxx | 2751 |
| HETATM | 2752 | O | HOH | S | 317 | 22.276 | −5.729 | 85.346 | 1.00 | 0.00 | xxxx | 2752 |
| HETATM | 2753 | O | HOH | S | 318 | 17.902 | 8.547 | 58.415 | 1.00 | 0.00 | xxxx | 2753 |
| HETATM | 2754 | O | HOH | S | 319 | 37.913 | 34.004 | 75.956 | 1.00 | 0.00 | xxxx | 2754 |
| HETATM | 2755 | O | HOH | S | 320 | 4.531 | 12.700 | 72.802 | 1.00 | 0.00 | xxxx | 2755 |
| HETATM | 2756 | O | HOH | S | 321 | 24.012 | −0.883 | 62.835 | 1.00 | 0.00 | xxxx | 2756 |
| HETATM | 2757 | O | HOH | S | 322 | 25.236 | −11.299 | 74.409 | 1.00 | 0.00 | xxxx | 2757 |
| HETATM | 2758 | O | HOH | S | 323 | 16.111 | −2.210 | 88.800 | 1.00 | 0.00 | xxxx | 2758 |
| HETATM | 2759 | O | HOH | S | 324 | 37.659 | −1.206 | 71.051 | 1.00 | 0.00 | xxxx | 2759 |
| HETATM | 2760 | O | HOH | S | 325 | 37.666 | −6.192 | 71.121 | 1.00 | 0.00 | xxxx | 2760 |
| HETATM | 2761 | O | HOH | S | 326 | 28.307 | 3.226 | 66.321 | 1.00 | 0.00 | xxxx | 2761 |
| HETATM | 2762 | O | HOH | S | 327 | 18.264 | 17.811 | 65.553 | 1.00 | 0.00 | xxxx | 2762 |
| HETATM | 2763 | O | HOH | S | 328 | 32.832 | 36.516 | 97.649 | 1.00 | 0.00 | xxxx | 2763 |
| HETATM | 2764 | O | HOH | S | 329 | 37.812 | 34.108 | 72.992 | 1.00 | 0.00 | xxxx | 2764 |
| HETATM | 2765 | O | HOH | S | 330 | 31.618 | −1.175 | 95.798 | 1.00 | 0.00 | xxxx | 2765 |
| HETATM | 2766 | O | HOH | S | 331 | 34.949 | −3.372 | 78.898 | 1.00 | 0.00 | xxxx | 2766 |
| HETATM | 2767 | O | HOH | S | 332 | 23.906 | −12.954 | 76.241 | 1.00 | 0.00 | xxxx | 2767 |
| HETATM | 2768 | O | HOH | S | 333 | 36.425 | 4.098 | 70.822 | 1.00 | 0.00 | xxxx | 2768 |
| HETATM | 2769 | O | HOH | S | 334 | 39.250 | 45.244 | 81.350 | 1.00 | 0.00 | xxxx | 2769 |
| HETATM | 2770 | O | HOH | S | 335 | 4.762 | 13.877 | 66.822 | 1.00 | 0.00 | xxxx | 2770 |
| HETATM | 2771 | O | HOH | S | 336 | 28.360 | 33.885 | 93.919 | 1.00 | 0.00 | xxxx | 2771 |
| HETATM | 2772 | O | HOH | S | 337 | 37.026 | −2.278 | 82.988 | 1.00 | 0.00 | xxxx | 2772 |
| HETATM | 2773 | O | HOH | S | 339 | 52.387 | 24.552 | 100.326 | 1.00 | 0.00 | xxxx | 2773 |
| HETATM | 2774 | O | HOH | S | 340 | 23.731 | 19.281 | 92.922 | 1.00 | 0.00 | xxxx | 2774 |
| HETATM | 2775 | O | HOH | S | 341 | 22.622 | 29.993 | 86.246 | 1.00 | 0.00 | xxxx | 2775 |
| HETATM | 2776 | O | HOH | S | 342 | 47.903 | 18.185 | 70.488 | 1.00 | 0.00 | xxxx | 2776 |
| HETATM | 2777 | O | HOH | S | 343 | 41.398 | 34.777 | 102.884 | 1.00 | 0.00 | xxxx | 2777 |
| HETATM | 2778 | O | HOH | S | 344 | 48.646 | 39.218 | 75.315 | 1.00 | 0.00 | xxxx | 2778 |
| HETATM | 2779 | O | HOH | S | 345 | 38.330 | 15.569 | 80.280 | 1.00 | 0.00 | xxxx | 2779 |
| HETATM | 2780 | O | HOH | S | 346 | 31.644 | 34.806 | 96.101 | 1.00 | 0.00 | xxxx | 2780 |
| HETATM | 2781 | O | HOH | S | 347 | 47.364 | 33.541 | 103.953 | 1.00 | 0.00 | xxxx | 2781 |
| HETATM | 2782 | O | HOH | S | 348 | 44.632 | 14.267 | 99.126 | 1.00 | 0.00 | xxxx | 2782 |
| HETATM | 2783 | O | HOH | S | 349 | 49.644 | 35.980 | 94.514 | 1.00 | 0.00 | xxxx | 2783 |
| HETATM | 2784 | O | HOH | S | 350 | 21.713 | 24.372 | 91.316 | 1.00 | 0.00 | xxxx | 2784 |
| HETATM | 2785 | O | HOH | S | 351 | 52.309 | 31.191 | 91.054 | 1.00 | 0.00 | xxxx | 2785 |
| HETATM | 2786 | O | HOH | S | 352 | 22.952 | 34.465 | 76.990 | 1.00 | 0.00 | xxxx | 2786 |
| HETATM | 2787 | O | HOH | S | 353 | 1.535 | 7.911 | 68.662 | 1.00 | 0.00 | xxxx | 2787 |
| HETATM | 2788 | O | HOH | S | 354 | 42.120 | 12.888 | 78.426 | 1.00 | 0.00 | xxxx | 2788 |
| HETATM | 2789 | O | HOH | S | 355 | 23.184 | −11.886 | 67.081 | 1.00 | 0.00 | xxxx | 2789 |
| HETATM | 2790 | O | HOH | S | 356 | 28.553 | 29.065 | 101.938 | 1.00 | 0.00 | xxxx | 2790 |
| HETATM | 2791 | O | HOH | S | 357 | 41.272 | 12.381 | 76.436 | 1.00 | 0.00 | xxxx | 2791 |
| HETATM | 2792 | O | HOH | S | 358 | 19.489 | 19.564 | 69.735 | 1.00 | 0.00 | xxxx | 2792 |
| HETATM | 2793 | O | HOH | S | 359 | 46.136 | 39.282 | 76.126 | 1.00 | 0.00 | xxxx | 2793 |
| HETATM | 2794 | O | HOH | S | 360 | 15.145 | 9.336 | 99.422 | 1.00 | 0.00 | xxxx | 2794 |
| HETATM | 2795 | O | HOH | S | 361 | 22.838 | 23.616 | 97.095 | 1.00 | 0.00 | xxxx | 2795 |
| HETATM | 2796 | O | HOH | S | 362 | 9.603 | 13.336 | 89.036 | 1.00 | 0.00 | xxxx | 2796 |
| HETATM | 2797 | O | HOH | S | 363 | 26.386 | −2.108 | 62.392 | 1.00 | 0.00 | xxxx | 2797 |
| HETATM | 2798 | O | HOH | S | 364 | 52.011 | 37.819 | 77.771 | 1.00 | 0.00 | xxxx | 2798 |
| HETATM | 2799 | O | HOH | S | 365 | 28.168 | 8.938 | 63.919 | 1.00 | 0.00 | xxxx | 2799 |
| HETATM | 2800 | O | HOH | S | 366 | 27.589 | 39.294 | 85.068 | 1.00 | 0.00 | xxxx | 2800 |
| HETATM | 2801 | O | HOH | S | 367 | 27.687 | −12.163 | 86.695 | 1.00 | 0.00 | xxxx | 2801 |
| HETATM | 2802 | O | HOH | S | 368 | 44.266 | 8.368 | 83.648 | 1.00 | 0.00 | xxxx | 2802 |
| HETATM | 2803 | O | HOH | S | 369 | 46.173 | 42.782 | 86.952 | 1.00 | 0.00 | xxxx | 2803 |
| HETATM | 2804 | O | HOH | S | 370 | 14.140 | 12.958 | 88.666 | 1.00 | 0.00 | xxxx | 2804 |
| HETATM | 2805 | O | HOH | S | 371 | 34.823 | 19.732 | 100.091 | 1.00 | 0.00 | xxxx | 2805 |
| HETATM | 2806 | O | HOH | S | 372 | 46.908 | 9.147 | 83.618 | 1.00 | 0.00 | xxxx | 2806 |
| HETATM | 2807 | O | HOH | S | 373 | 24.142 | 29.305 | 94.702 | 1.00 | 0.00 | xxxx | 2807 |
| HETATM | 2808 | O | HOH | S | 374 | 18.119 | −1.328 | 89.006 | 1.00 | 0.00 | xxxx | 2808 |
| HETATM | 2809 | O | HOH | S | 375 | 33.026 | 38.391 | 92.683 | 1.00 | 0.00 | xxxx | 2809 |
| HETATM | 2810 | O | HOH | S | 376 | 58.201 | 22.385 | 92.378 | 1.00 | 0.00 | xxxx | 2810 |
| HETATM | 2811 | O | HOH | S | 377 | 54.105 | 29.892 | 90.670 | 1.00 | 0.00 | xxxx | 2811 |
| HETATM | 2812 | O | HOH | S | 378 | 34.565 | 8.799 | 95.893 | 1.00 | 0.00 | xxxx | 2812 |
| HETATM | 2813 | O | HOH | S | 379 | 27.908 | 0.262 | 61.279 | 1.00 | 0.00 | xxxx | 2813 |
| HETATM | 2814 | O | HOH | S | 380 | 19.941 | 22.488 | 82.984 | 1.00 | 0.00 | xxxx | 2814 |
| HETATM | 2815 | O | HOH | S | 381 | 29.525 | −1.794 | 97.959 | 1.00 | 0.00 | xxxx | 2815 |
| HETATM | 2816 | O | HOH | S | 382 | 49.648 | 29.585 | 104.557 | 1.00 | 0.00 | xxxx | 2816 |
| HETATM | 2817 | O | HOH | S | 383 | 35.408 | 8.346 | 93.778 | 1.00 | 0.00 | xxxx | 2817 |
| HETATM | 2818 | O | HOH | S | 384 | 52.470 | 22.521 | 101.253 | 1.00 | 0.00 | xxxx | 2818 |
| HETATM | 2819 | O | HOH | S | 385 | 26.897 | −3.606 | 89.300 | 1.00 | 0.00 | xxxx | 2819 |
| HETATM | 2820 | O | HOH | S | 386 | 47.051 | 43.266 | 84.658 | 1.00 | 0.00 | xxxx | 2820 |
| HETATM | 2821 | O | HOH | S | 387 | 27.492 | −1.155 | 98.711 | 1.00 | 0.00 | xxxx | 2821 |
| HETATM | 2822 | O | HOH | S | 388 | 50.334 | 39.736 | 96.998 | 1.00 | 0.00 | xxxx | 2822 |
| HETATM | 2823 | O | HOH | S | 389 | 53.977 | 16.042 | 91.468 | 1.00 | 0.00 | xxxx | 2823 |
| HETATM | 2824 | O | HOH | S | 390 | 55.332 | 22.862 | 98.469 | 1.00 | 0.00 | xxxx | 2824 |
| HETATM | 2825 | O | HOH | S | 391 | 55.356 | 15.678 | 95.126 | 1.00 | 0.00 | xxxx | 2825 |
| HETATM | 2826 | O | HOH | S | 392 | 29.452 | 26.421 | 74.947 | 1.00 | 0.00 | xxxx | 2826 |

### Example 13. Robust, Thermostable, Reagentless, Fluorescently Responsive Glucose Biosensors

We report the construction of a robust, thermostable, reagentless, fluorescently responsive glucose biosensor and its variants derived from T. thermosaccharolyticum (ttGGBP). These proteins are useful for high-precision chemometric measurements that span the entire clinical and industrial glucose concentration range, using fluorescence ratiometry measured with straightforward, inexpensive instrumentation.

Thermostable homologs of the E. coli Glucose-galactose binding protein (ecGGBP) were identified using a bioinformatics search strategy that applied a structure-based sequence filter to identify the subset of sequences that retain the original function within the larger collection of aligned sequence homologs. The homologs of interest appeared at sequence identities below 60% of the ecGGBP probe an unusual and surprising discovery. At this level, overall identities are weak predictors of biological function, application of the structure-based filter therefore was essential for accurate identification. The glucose-binding properties of the predicted hits were tested experimentally by constructing synthetic genes optimized for heterologous protein expression in E. coli and determining the glucose-binding properties of the expressed proteins. This search resulted in the identification of a homolog from Thermoanaerobacter thermosaccharolyticum (ttGGBP) as a suitable candidate for glucose sensor engineering.

Endosterically placed Acrylodan and Badan fluorescent conjugates were found to be highly effective ratiometric glucose sensors. A series of additional mutations were introduced to manipulate glucose affinities. Variants spanning four orders of magnitude (0.1-100 mM) were identified. Within these, a subset of mutants covers the entire pathophysiological glucose concentration range with responses that remain within 90% of the maximally achievable precision.

The ttGGBP-based FRSs can be immobilized site-specifically on magnetic beads or other solid or semi-solid substrates without affecting protein stability, fluorescence responses, or glucose affinities. They can be dried, and aged aggressively (incubation at 50° C. for 7 days or more, e.g. mimicking and/or exceeding environmental conditions in the field, storage or shipping) without adversely affecting sensing performance.

Reagentless, fluorescently responsive sensors present a number of advantages over enzyme-based biosensors, including self-calibration, elimination of chemical transformations and multiple substrates, which together lead to simple sample-handling fluidic circuitry and rapid response times. FRSs can be used for one-time, episodic, and continuous monitoring measurements. Additionally, the use of robust engineered glucose sensors based on thermophilic proteins is likely to simplify manufacturing and distribution processes. Combination of mutant glucose sensors reported here into multiplexed arrays or composites determine glucose concentrations from hypoglycemic to the hyperosmolar hyperglycemic state samples with high precision in one measurement. Such systems have significant potential for the development of next-generation high-accuracy, wide dynamic range sensing applications in continuous monitoring, point-of-care, or wearable systems.

The Following Materials and Methods were Used to Generate the Data Described Herein.

Bioinformatic searches. Annotated genomic and plasmid sequences of 4592 prokaryotes were downloaded from the National Center of Biotechnology Information (ftp://ftp.ncbi.nih.gov/genomes/Bacteria/all.gbk.tar.gz), together with annotations recording prokaryotic lifestyles (../ProkaryotesOrganismInfo.txt). We developed the ‘ProteinHunter’ program to provide an interface and methods for organizing, querying, and analyzing these genomic sequences as well as protein structures. ProteinHunter comprises a graphical user interface, set of computer scripts, and a parallel computing environment. Together these set up the calculations, manage the flow of information and execution in each of the calculation phases, control other programs that carry out specific calculations such as BLAST and ClustalW, and visualize the results. The protein sequence for the E. coli glucose-galactose binding protein (ecGGBP) was extracted from the protein structure file 2gbp (Vyas et al. 1988 Science, 242, 1290-5), and used as the seed sequence for a uni-directional BLAST search of the downloaded prokaryotes. Pairwise BLAST alignment were selected in ProteinHunter as hits if these contained 25% or identical residues, and if the alignment covered at least 70% of the probe and target sequences. The BLAST hits identified in ProteinHunter were aligned using ClustalW (Chenna et al. 2003 Nucleic Acids Res, 31, 3497-500). A structure-based sequence filter was used to accurately distinguish glucose-binding protein from other functions within the hits. A 10-residue, non-contiguous sequence comprising the primary complementary surface (PCS) between the protein and the bound glucose in the 2gbp structure (FIG. 1A-C) was identified using ProteinHunter. PCS residues were selected as members of the PCS if the calculated distance between any of their atoms and any glucose atom was less than 5 Å, and the distances between their backbone C_{α} and any atom in glucose was greater than that of their C_{β} atom and any atom in glucose. Secondary shell residues that do not form hydrogen bonds or van der Waals contacts were removed by inspection from the resulting set. Using the overall ClustalW alignments, the subset of PCS residues were identified within each homolog and aligned. For each homolog, the number of PCS mutations relative to the ecGGBP PCS (Hamming distance, H_{PCS}) was counted. A H_{PCS}=0 value means that the PCS sequence is identical to that of eCGGBP; homologs with this H_{PCS} value were inferred to be glucose-binding proteins. The PCS sequences were displayed sorted by their H_{PCS} values, and within each H_{PCS} value sorted by their fraction identical residues, indicating the replicon within which they reside (chromosome or plasmid), whether this replicon contains paralogs, and the temperature tolerance (hyperthermophile, thermophile, mesophile, psychrophile, unknown), their Gram stain classification (if known), and the percentage genomic AT content. Duplicate hits were removed automatically from this list if the organism name (genus and species), fractional identity and paralogs were the same. From this list unique hyper(thermophilic) ecGGBP homologs with H_{PCS}=0 were readily identified by inspection.

Gene synthesis and mutagenesis. The amino acid sequences for the GGBP homologs identified in the bioinformatic search (see above) were extracted from the ClustalW alignment file and edited further to construct a mature polypeptide with a single cysteine that replaces the equivalent of W183 in ecGGBP for site-specific labeling with Acrylodan, using AaEditor, an in-house program developed to manipulate protein sequences. The putative leader peptide that mediates anchoring of the periplasmic-binding protein on the outside of the membrane (Gram positive bacteria) or directs secretion into the periplasm (Gram negative bacteria) was deleted by examining the multiple sequence alignment and removing the sequences N-terminal to the start of the mature ecGGBP amino acid sequence. The residue equivalent to W183 in the ecGGBP sequence was mutated to cysteine; all other cysteines were changed to alanine. A hexahistidine tag was placed behind a GGS linker at the C-terminus of the mature protein to enable metal-mediated affinity purification (Hengen 1995 Methods Enzymol, 210, 129-192). The final amino acid sequence was back-translated into a DNA sequence encoding the open reading frame (ORF), which was placed in a construct behind an efficient Shine-Dalgarno ribosome-binding site, and flanked by a T7 promoter and terminator at the 5′ and 3′ ends respectively, using the GeneFab program (Cox et al. 2007 Protein Sci, 16, 379-90). The resulting ORF sequence was optimized in context by OrfOpt or OrfMorph programs designed to predict highly expressed mRNA sequences in E. coli. The resulting DNA sequences were synthesized by oligonucleotide assembly and cloned into pUC57 by GeneWiz, Inc. (South Plainfield, New Jersey). Subsequent single and multiple point mutations were constructed by preparing mutant sequences of the synthetic ORF sequences using GfMutagenesis, an in-house program that introduces point mutations into an ORF using the most prevalent codon in E. coli for an amino acid, followed by total gene synthesis.

Synthetic gene optimization. The OrfOpt and OrfMorph programs use stochastic optimization algorithms that alter choose different codons within an ORF without altering the amino acid sequence to optimize a target function designed to identify mRNA sequences that express proteins at high levels in E. coli. The OrfOpt simultaneously imposes AU-rich nucleotide composition at the 5′ and 3′ ends of the ORF, low RNA secondary structure content and favorable codon usage (Allert et al. 2010 J Mol Biol, 402, 905-18). The OrfMorph program reproduces the pattern of codon usage and RNA secondary observed in the parent genome of a protein, but using E. coli codon preferences and nucleotide composition.

Codon usage is calculated using the codon adaptation index (CAI), as described for OrfOpt, using codon frequency tables calculated for the genome under examination. The mean CAI value for a genome, μ_{c}, and its standard deviation, σ_{c}, are calculated over all the codons in a genome. A codon usage score, c, is calculated for each codon in an open reading frame (ORF) by averaging the CAI over a 9-codon window, centered on the codon for which this score is calculated. A normalized codon usage score, z_{c}, is calculated for each codon as the Z-score: z_{c}=(c−μ_{c})/σ_{c}. A plot of z_{c} along an ORF establishes the codon usage pattern of that ORF. Rare codons (z_{c}<0) are hypothesized to slow down the elongation rate of ribosome translation, introducing “pause” sites at extremes. Such pause sites are hypothesized to direct the kinetics of co-translational protein folding, allowing a newly synthesized segment to fold before more protein is made. An RNA secondary structure score, s, is determined for each nucleotide by summing its participation in all possible hairpins that can form in its vicinity (settings: minimum stem duplex length, 4 basepairs; maximum loop length, 30 bases; vicinity length, 100 bases), as described for OrfOpt. The average secondary structure energy, μₛ, and its standard deviation, σₛ, are calculated over all the nucleotides in a genome. A normalized secondary structure energy score, zₛ, is calculated for each codon as the Z-score: zₛ=(s−μₛ)/σₛ. A plot of zₛ along an ORF establishes the secondary structure pattern of that ORF. Regions of above-average secondary structure (zₛ>0) are hypothesized to slow down the elongation rate of ribosome translation, introducing “pause” sites at extremes. As with CAI-mediated pause sites, secondary structure-driven pause sites are hypothesized to direct the kinetics of co-translational protein folding. To imitate these patterns for heterologous expression of an ORF in E. coli, first the z_{c} and zₛ scores are calculated using the parent organism codon table, μ_{c}, σ_{c}, μₛ, and σₛ values. Second, a stochastic search algorithm is used that randomly chooses between degenerate codons to construct trial mRNA nucleotide sequences, calculating z_{c} and zₛ scores for each trial sequence, but using the E. coli codon table, and E. coli μ_{c}, σ_{c}, μₛ, and σₛ values. For each trial sequence, the absolute differences between the E. coli trial scores, and the wild-type scores are summed over the entire ORF. The OrfMorph program searches for a minimum of these differences. The stochastic search algorithm operates by first choosing a codon position, second choosing a degenerate codon within the allowed codons at that position. If the choice results in an improved score, the sequence is kept, otherwise it is rejected. After a position has been selected, it is removed from the pool of allowed positions, and the next is chosen from the remained. A “sweep” is completed, when all the codons in the sequence have been examined. The algorithm terminates, when two successive sweeps do not yield further improvements in the score. The resulting RNA nucleotide sequence then has codon usage patterns and secondary structure patterns that closely match those of the wild-type mRNA sequence in its parental genomic context. The hypothesis is that such matching improves production of soluble protein by mimicking co-translation folding contributions that minimize mis-folded protein intermediate aggregation.

Protein expression, purification, and fluorescent conjugate preparation. Plasmids carrying the expression constructs (see above) were transformed into KRX competent cells (Promega), and grown overnight at 37° C. on LB agar plates (100 mg/mL ampicillin). A single colony was picked and grown overnight at 37° C. in Terrific Broth (TB; Research Products International). The overnight cultures were diluted 1:20 in 500 mL TB (100 mg/mL ampicillin, 1 mM CaCl₂)), grown to an optical density of A₆₀₀=0.5 at 37° C. in vigorously aerated shaker flasks, induced by the addition of 2.5 mL rhamnose (20% w/v), and grown for a further 3-4 hrs. The cells were harvested by centrifugation (5,000 rpm, 10 min). After decanting the supernatant, the cell pellets were stored −80° C. The cell pellets were thawed, resuspended in 8 mL binding buffer (10 mM imidazole, 20 mM MOPS, 500 mM NaCl, 1 mM CaCl₂), pH 7.8). Following resuspension, 3 mL of BugBuster HT (EMD Millipore) was added. After incubation (20 mins, 25° C.), the cells were lysed on ice by sonication (2 minutes of one-second on/off pulses, 20-30% power). A clarified lysate was prepared by centrifugation (15,000 rpm, 20 min, 4° C.) from which recombinant protein was purified by batch immobilized metal affinity chromatography (IMAC). Resuspended IMAC agarose beads (5 mL; Sigma-Aldrich, P6611) were added to the lysate. After incubation at 4° C. in a Mini LabRoller (Labnet International) for 1 hr, the beads were washed at least five times with binding buffer. The immobilized protein beads were resuspended in labeling buffer (20 mM MOPS, 100 mM NaCl, 1 mM CaCl₂), pH 6.9) and labeled overnight (4° C., rotating end-over-end) with a thiol-reactive fluorophore (5-fold stoichiometric excess over protein). Following two rinses with labeling buffer to remove unincorporated label, the proteins were eluted from the beads. To elute labeled protein from the IMAC beads, 6 mL of elution buffer (400 mM imidazole, 500 mM NaCl, 1 mM CaCl₂), 20 mM MOPS, pH 7.8) was added, incubated for 30 min (4° C., rotating end-over-end), and the beads removed by centrifugation. Following dialysis of the eluate against three changes of assay buffer (20 mM MOPS, 20 mM KCl, 1 mM CaCl₂), pH 7.4), using 10 kDa semi-permeable membrane (Snakeskin tubing, Thermo Scientific), the fluorescent conjugates were concentrated in a 10 kDa cutoff spin concentrator (Vivaspin, GE Healthcare). Protein purity was assessed by SDS/PAGE. Protein concentrations were determined by (Nanodrop1000) at 280 nm (using extinction coefficients calculated from their sequence (Gill and von Hippel 1989 Anal Biochem, 182, 319-26; Artimo et al. 2012 Nucleic Acids Res, 40, W597-603), or at the fluorophore absorbance peak (Acrylodan, 391 nm and Badan, 387 nm).

Determination of temperature- and ligand-dependent fluorescence landscapes. 12-, 24-, or 48-point logarithmic titration series were prepared on a Tecan Freedom liquid-handling robot, using an in-house program, ‘TitrationPlate’, that compiles an abstract description of a multi-component titration series into machine instructions for operating the robot. Glucose concentrations were varied from 0-1.7 M in 20 mM KCl, 20 mM MOPS (pH 7.4) supplemented with either 1 mM EGTA or 1 mM CaCl₂). Temperature-dependent fluorescence emission intensities of 20 μL aliquots, each containing 10 UM protein, were measured in 384-well microtiter plates in a LightCycler 480 II (Roche) using excitation and emission wavelengths available for this instrument that most closely matched the optical characteristics of the fluorescent conjugate. Temperatures were advanced in 1K steps. At each temperature, data was collected at 1-second intervals for 60 seconds at which point the signal had relaxed to a steady value associated with the new temperature. Under these experimental photobleaching was not observed. The in-house program ‘TitrationMeltPlate’ was used to convert these observations into time-independent datasets that record fluorescence as a function of temperature for each well and associate wells with their concentration of titrant and additive. Management tools were developed to maintain a database of titrations and their analyses.

Determination of emission intensity spectra. Ligand- and wavelength-dependent emission intensities were recorded on a Nanodrop3300 (Thermo Scientific) at room temperature. Using the LED closest to the optimal excitation wavelength of the fluorophore (UV, 365 nm; blue, 470 nm; ‘white’, 460-550 nm).

Ratiometric analysis of glucose binding. Isothermal glucose titrations were extracted from the fluorescent landscape or emission spectra datasets obtained as described above.

Monochromatic emission intensities I_{λ} (these intensities correspond to a bandpass intensity, recorded either with a physical filter in the case of the Roche LightCycler, or by integrating in the interval 2λ−δ, λ+δ in the case of an emission spectrum), were fit to

I_{λ}=^{apo}β_{λ}(1−y̅ₜᵣᵤₑ)+^{sat}β_{λ}y̅ₜᵣᵤₑ 1

where ^{apo}β_{λ} and ^{sat}B_{λ} are the fluorescence baselines associated with the ligand-free and ligand-bound states of the protein, respectively, and y̅ₜᵣᵤₑ the fractional saturation of the protein. (Layton and Hellinga 2010 Biochemistry, 49, 10831-41.) Baseline functions can be constant, linear, or a second-order polynomial. For the ligand- and temperature-dependent fluorescence landscapes, we use a constant value for ^{apo}βₓ, but ^{sat}βₓ is described by a linear dependence on glucose concentration, [L]:

^{sat}βₓ=aₓ+bₓ[L] 2

For a single glucose-binding site, the fractional saturation is given by

$$\overline{y} = \frac{[ L ]}{[ L ] + {K}_{d}} \qquad 3$$

where [L] is the ligand (glucose) concentration and K_{d} the dissociation constant, ^{true}K_{d} for y̅ₜᵣᵤₑ.

A dichromatic, ratiometric signal is defined as the ratio of the intensities at two independent wavelengths, λ₁ and λ₂

R_{1,2}=I_{λ1}/I_{λ2} 4

This signal removes wavelength-independent emission intensity attenuation effects due to variations in conjugate concentration, photobleaching, fluctuations in excitation source intensities, and detection efficiency. (Demchenko 2010 J Fluoresc, 20, 1099-128; Demchenko 2014 Journal of Molecular Structure, 1077, 51-67) It is a key aspect for high-precision sensing using the reagentless fluorescently-responsive sensors described here. The ratiometric signal also can be fit to a binding isotherm:

R_{1,2}=^{apo}β_{R}(1−y̅_{R})+^{sat}β_{R}y̅_{R} 5

where ^{apo}β_{R} and ^{sat}β_{R} are the baselines, and y̅_{R} the apparent fractional saturation of the protein (with ^{app}K_{d}). In general, ^{true}K_{d}≠^{app}K_{d}; if both baselines are constant, a simple relationship can be derived relating ^{app}K_{d} to ^{true}K_{d}: (Grimley et al. 2013 J Neurosci, 33, 16297-309)

$${app}^{} {K}_{d} {=}^{true} {K}_{d} \frac{{apo}^{} {I}_{λ2}}{{sat}^{} {I}_{λ2}} \qquad 6$$

where ^{apo}I_{λ2} and ^{sat}I_{λ2} are the emission intensities of the monochromatic signal at wavelength λ₂ of the ligand-free and ligand-bound protein, respectively.

The fractional error in the chemometric concentration measurement, depends on the first derivative of the binding isotherm as follows (Marvin et al. 1997 Proc Natl Acad Sci USA, 94, 4366-71):

$$\frac{\partial S}{S} = \frac{{ε}_{1 , 2}}{S} \times {( \frac{d y {R}_{1 , 2}}{d S} )}^{- 1} \qquad 7$$

Where R_{1,2} is the ratiometric signal (equation 5), ε_{1,2} its experimental error, and δS is the resulting chemometric error in the concentration. We can then define a relative precision function

$$P ( S ) = \frac{S}{δ S} \times \frac{1}{{P}_{max}} \qquad 8$$

where P(S) is the relative precision at concentration S, which reaches a maximum value (i.e. lowest error), Pₘₐₓ, at the K_{d}.

For a given isothermal titration, values for ^{app}K_{d} and ^{true}K_{d} were obtained using a non-linear fitting algorithm in which these two parameters were simultaneously fit to the three experimental binding isotherms using equations 1 and 5, with the two monochromatic isotherms sharing the same ^{true}K_{d} value. Three separate pairs of ^{apo}β and ^{sat}β were fit in this procedure. Programs ‘Nanodrop3300’ and ‘TitrationMeltAnalysis’ were developed to analyze wavelength- or temperature-dependent ligand-binding datasets respectively.

Analysis of temperature- and ligand-dependent fluorescent landscapes. To obtain the temperature dependence of the binding reaction, the K_{d} values of all the individually determined isotherms were fit the Gibbs-Hemholtz equation (Layton and Hellinga 2010 Biochemistry, 49, 10831-41):

$$Δ {G}_{b}^{\cdot } ( T ) = {Δ}^{r e f} {H}_{b}^{\cdot } + Δ {C}_{p , b} ( T - {T}_{r e f} ) - T ( {Δ}^{r e f} {S}_{b}^{\cdot } + Δ {C}_{p , b} ln \frac{T}{{T}_{r e f}} ) \qquad 9$$

where ΔG_{b}^{⋅}(T) is the standard free energy of binding at 1 M ligand at temperature T,

$$Δ {G}_{b}^{\cdot } ( T ) = - RT ln ( 1 + \frac{1}{{K}_{d} ( T )} ) \qquad 10$$

Δ^{ref}H_{b}^{⋅}; and Δ^{ref}S_{b}^{⋅} the molar enthalpy and entropy of binding, respectively, at the reference temperature, T_{ref}, and ΔC_{p.b} the heat capacity of the binding reaction. This data analysis was carried out using ‘TitrationMeltAnalysis’.

Analysis of emission spectra components. Wavelength-dependent, I(λ), emission intensities at were converted to wavenumber-dependent intensities (Valeur 2012 Principles and Applications. Weinheim: Wiley; Lakowicz 2006 Principles of fluorescence spectroscopy. Springer, New York), I(ν):

I(ν)=λ²I(λ) 11

Singular value decomposition was used for model-free identification of regions in the emission spectra that vary with respect to glucose concentration (Henry 1992 Methods Enzymol, 210, 129-192). An Aₘₙ data matrix was constructed by recording I(ν) values of m frequencies in columns for n titration points in rows. This matrix was decomposed as

Aₘₙ=UₘₙSₙₙVₙₙ^{T} 12

where Uₘₙ records n spectral components at m frequencies ranked by the weight of their contribution to the reconstruction of the experimental data, Vₙₙ records the contribution of the nth component to the n^{th} titration point, Sₘₙ records the weight of the n^{th} component. Decomposition was carried using the in-house Nanodrop3300 program, written in Python. The linalg.svd method in the open-source Python scipy package (www.scipy.org, version 0.7.2) was used to solve the decomposition. The relative weight of the n^{th} component in Uₘₙ, fₙ, was calculated from Sₙₙ, by normalizing the values in S with its trace:

$${f}_{n} = \frac{{S}_{n n} ( n , n )}{t r ( {S}_{n n} )} \qquad 13$$

The fractional states of n individual electronic transitions in a spectrum were determined by fitting n Gaussians (Valeur 2012 Principles and Applications. Weinheim: Wiley; Lakowicz 2006 Principles of fluorescence spectroscopy. Springer, New York) to the emission intensities of the corrected spectra (equation 5) transformed into the frequency domain (equation 6):

$${calc}^{} {I}_{i} ( v ) = {\sum }_{i = 1}^{i = n} \frac{{A}_{i}}{\sqrt{{2}_{π}}} {e}^{- \frac{1}{2} {( \frac{v - {μ}_{i}}{σ} )}^{2}} \qquad 14$$

where μᵢ is the wavenumber corresponding to the peak intensity of the i^{th} transition, Aᵢ the area contributed to the total spectrum by this transition, and σ the spectral width of all transitions. The fraction, fᵢ, of the i^{th} transition is given by:

$${f}_{i} = \frac{{A}_{i}}{{\sum }_{j = 1}^{j = n} {A}_{j}} \qquad 15$$

Wavelength dependent residuals are given by:

Δ(ν)−^{obs}I(σ)−^{calc}I(ν) 16

Fits were carried out by minimizing the least squares difference between observed and calculated spectra, using simplex and conjugate gradient methods implemented in Nanodrop3300 (scipy package methods optimize.fmin and optimize.leastsq, respectively). For titration series with N spectra, collected as a function of titrant concentration, global fits were used in which, as a first approximation, μi values were kept identical in the apo-protein and saturated glucose complex, and σ was universal for all transitions in all spectra. A_{i,k} values were allowed to vary in each k^{th} spectrum. The variation of the fraction for each transition, f_{i,k}, was then fit to a binding isotherm (equation 1), constraining the fit ^{app}K_{d} value to be common to all transitions.

Structure determination by X-ray crystallography. Glucose-binding protein fluorescent conjugates were mixed with 2 mM D-glucose, and 1 mM CaCl₂). Sitting-drop vapor-diffusion Crystallization trials were carried out at 17° C. using sparse-matrix screening conditions. The ecGGBP183C⋅Acrylodan, ttGGBP182C⋅Acrylodan and ttGGBP.17C⋅Badan conjugates crystallized in 20% PEG 3350 and 0.2 M potassium thiocyanate as clusters of needles. Single crystals were isolated mechanically (Micro-Tools, Hampton Research), transferred stepwise into mother liquor containing 30% ethylene glycol, and flash-frozen in liquid nitrogen. Diffraction data was collected remotely at the Advanced Photon Source, SER-CAT beamline 22-ID; 0.5° oscillation angle frames were collected and processed using XDS program (Kabsch 2010 Acta Cryst., D66, 125-132). The data was phased by molecular replacement using PHASER (MCCoy 2007 J. Appl. Cryst., D66, 125-312) with a poly-alanine of the E. coli glucose-galactose-binding protein (2gbp (Vyas et al. 1988 Science, 242, 1290-5)) as the search model. Initial models were built after 10 cycles of rigid-body refinement in PHENIX, AutoBuild (Adams 2010 Acta Crystallogr D Biol Crystallogr, 66, 213-331). Multiple rounds of positional, individual B-factor and occupancy refinement and subsequent model building were performed in PHENIX and COOT, respectively. Solvent molecules were added both automatically as implemented in phenix.refine and by manual inspection. The structures were validated using PHENIX tools. The final refined models has crystallographic R_{factor} and R_{free} values that were within the range of average values refined at these resolutions (Kleywegt 1996 Structure, 4, 897-904).

### Example 14. FRS Uses

The glucose sensors can be incorporated into point-of-care clinical devices to measure glucose concentrations accurately, and rapidly at the patient bedside. In such a device, a small blood sample (<10 μL) is obtained by means of a finger stick using a lancet. This sample droplet is then placed on the aperture of a disposable cartridge containing desiccated, immobilized glucose sensors inside a small measurement chamber. The sample enters the chamber by virtue of passive capillary action, wetting the sensors upon contact. As soon as the sensors have been wetted, they bind glucose, and report on its concentration by virtue of the engineered fluorescent sensor mechanism. The cartridge is placed inside a small reader (handheld or on a desktop), and their fluorescence signal is measured by the (inexpensive) optoelectronic components of the reader. Excitation light is provided by a light-emitting diode (LED. In the case of Acrylodan or Badan, a commercially available 400 nm blue LED is used, and the emitted light is measured through two bandpass filters. Cartridges can contain multiple sensors, spanning the entire clinical range of possible glucose concentrations. Each sensor is immobilized at a particular, known location inside the cartridge, providing “spatial addressability”. The intensity at a particular wavelength is then recorded by imagining these sensors using an inexpensive camera, such as a Complementary metal-oxide semiconductor (CMOS) device commonly found in consumer electronics such as cell phones. Each pixel in the camera records the emitted light on a gray scale. Integration of that signal imaged through the two signals, is analyzed by an on-board computer to calculate the ratiometric signal for each immobilized sensor. Pre-recorded hyperbolic binding curves are then used to calculate the glucose concentration in the sample. Recording through multiple sensors, tuned for accurate detection at different glucose concentrations provides a high-accuracy reading. This process is completed in less than a minute.

Similar instrumentation can be used for any type of episodic measurements, for instance, using other bodily fluids, or samples obtained from animals, or non-biological samples such as foods and beverages.

The FRS glucose sensors also can be used to monitor glucose levels continuously. For instance, sensors can be immobilized at the tip of a thin optical fiber to construct a glucose-responsive optode. Such an optode can be introduced into the body subcutaneously, using a small needle. Excitation and emission light are passed to and from the immobilized sensor, respectively. The sensor is in continuous contact with the sample. Fluctuations in the glucose sample alter the dynamic equilibrium between the open and closed states of the glucose-binding protein, which is transduced into fluctuations of the fluorescent emission signal, by virtue of the sensing mechanism of the conjugated fluorophore. The emitted light intensities are read through filters by a reader connected to the optode. This reader continuously displays the change in signal, and the corresponding calculated glucose concentrations. Continuous glucose monitoring accomplished using a device containing the immobilized glucose biosensor(s), e.g., a fiber optic biosensor, introduced into the subject intradermally or subcutaneously (Judge et al., 2011, Diabetes Technology & Therapeutics 13 (3):309-317; Weidemaier et al., 2011, Biosensors and Bioelectronics 26:4117-4123; hereby incorporated by reference). For example, subcutaneously placed sensors can be used to monitor the glucose levels in a patient, guiding automated delivery of insulin in an artificial pancreas.

Similar instrumentation can be used to monitor glucose levels in a fermentor or bioreactor, coupled to automated injection of glucose for growth optimization of bacteria, fungi, and eukaryotic cells.

As was discussed above, the features that distinguish the described constructs, devices, and methods from earlier glucose assay systems include:
Self-calibration
Rapid response time
Simple sample-handling fluidic circuitry
No additional components/substrates (“reagentless”)
No incubation time to develop signal. Reading is near-instantaneous and continuous
Stability (simplifies manufacturing, distribution, storage)
Small sample volume (<10 μL).
Capable of precise measurements over extended glucose concentration range (from the hypoglycemic to the hyperglycemic-hyperosmotic range)
Multiple sensors also provides redundancy, lowering error
Large scope of uses: episodic, continuous, ex vivo, in vivo, optodes, implants.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1-82. (canceled)

83. A biosensor for glucose comprising a glucose-galactose binding protein (GGBP) and a reporter group that transduces a detectable signal, wherein said reporter group is attached to said glucose-galactose binding protein and wherein said glucose-galactose binding protein is a mutant of Escherichia coli (E. coli) GGBP, wherein said glucose-galactose protein comprises an amino acid sequence having at least 90% identity to the amino acid sequence of SEQ ID NO: 16, 17, 32, 33 or 297 and retaining at least 90% of the activity of a naturally occurring E. coli glucose-galactose binding protein (ecGGBP).

84. The biosensor of claim 83, wherein said glucose-galactose-binding protein comprises one or more of the following substitutions: Y10X, D14X, N15X, F16X, P70X, N91X, K92X, S112X, S115X, E149X, H152X, P153X, D154X, A155X, R158X, M182X, W183X, N211X, D212X, D236X, L238X, D257X, P294X, and V296X, where X is any amino acid, an amino acid that results in a conservative substitution, or a cysteine, and wherein each position is counted in ecGGBP without including the signal peptide (SEQ ID NO: 17).

85. The biosensor of claim 84, wherein X is cysteine.

86. The biosensor of claim 83, wherein said glucose-galactose binding protein comprises a mutation at position 183 and/or position 16, and wherein each position is counted in ecGGBP without including the signal peptide (SEQ ID NO: 17).

87. The biosensor of claim 83, wherein said reporter group is conjugated to a cysteine at one of positions 183 or 16, and wherein each position is counted in ecGGBP without including the signal peptide (SEQ ID NO: 17).

88. The biosensor of claim 83, wherein glucose-galactose binding protein (ecGGBP) comprises a primary complementary surface (PCS), wherein said PCS comprises residues selected from the group consisting of Y10, D14, N15, F16, N91, K92, E93, S112, S115, E149, H152, D154, A155, R158, M182, W183, N211, D236, L255, N256, D257, P294, or V296, and wherein each amino acid position is numbered as in (SEQ ID NO: 17).

89. The biosensor of claim 83, wherein said glucose-galactose protein has no deletions or insertions compared to the naturally occurring protein.

90. The biosensor of claim 83, wherein said glucose-galactose protein comprises (i) less than about 5, 4, 3, 2, or 1 inserted amino acids, and/or (ii) less than about 5, 4, 3, 2, or 1 deleted amino acids compared to the naturally occurring protein.

91. The biosensor of claim 83, wherein said mutant comprises a mutation that alters the mutant's affinity and/or specificity for glucose compared to said naturally occurring E. coli glucose-galactose binding protein (ecGGBP).

92. The biosensor of claim 83, wherein said reporter group comprises a fluorophore, and wherein said signal comprises a fluorescent signal.

93. The biosensor of claim 92, wherein an emission spectrum of said fluorophore exhibits hypsochromicity or bathochromicity upon ligand binding to the ligand-binding domain of said ligand-binding protein.

94. The biosensor of claim 92, wherein said fluorophore comprises 5-iodoacetamidofluorescein (5IAF) or 6-iodoacetamidofluorescein (6IAF), rhodamine, Oregon Green, eosin, Texas Red, indocarbocyanine, oxacarbocyanine, thiacarbocyanine, merocyanine, Badan, Acrylodan, IAEDANS, comprising 3-cyano-7-hydroxycoumarin, 7-hydroxycoumarin-3-carboxylic acid, 6,8-difluoro-7-hydroxy-4-methylcoumarin, or 7-amino-4-methylcoumarin, pyridyloxazole, nitrobenzoxadiazole, benzoxadiazole, DRAQ5, DRAQ7, or CyTRAK Orange, cascade blue, Nile red, Nile blue, cresyl violet, oxazine 170, proflavin, acridine orange, acridine yellow, auramine, crystal violet, malachite green, porphin, phthalocyanine, bilirubin, pyrene, N,N′-dimethyl-N-(iodoacetyl)-N′-(7-nitrobenz-2-ox-a-1,3-diazol-4-yl)ethylenediamide (NBD), N-((2-(iodoacetoxy)ethyl)-N-methy-1)amino-7-nitrobenz-2-oxa-1,3-diazole (NBDE), JPW4039, JPW4042, JPW4045, Oregon Green, Pacific Blue, N,N′-Dimethyl-N-(Iodoacetyl)-N′-(7-Nitrobenz-2-Oxa-1,3-Diazol-4-yl)Ethylenediamine (IANBD), 7-diethylamino-3-(4′-maleimidylphenyl)-4-methylcoumarin (CPM), BODIPY 499, BODIPY 507, Alexa488, Alexa532, Alexa546, Cy5, or 1-(2-maleimidylethyl)-4-(5-(4-methoxyphenyl)oxazol-2-yl)pyridinium methanesulfonate (PyMPO maleimide) (PyMPO).

95. The biosensor of claim 94, comprising Acrylodan (6-acryloyl-2-dimethylaminonaphthalene) or Badan (6-bromo-acetyl-2-dimethylamino-naphthalene) or a derivative thereof.

96. The biosensor of claim 95, wherein said derivative comprises a replacement of the two-ring naphthalene of Acrylodan or Badan with a three-ring anthracene, a fluorene, or a styrene.

97. A method of detecting the presence of glucose in a sample, the method comprising: (a) contacting the biosensor of claim 83 with the sample; (b) measuring a signal from the biosensor; and (c) comparing the signal to a glucose-free control, wherein a difference in signal indicates the presence of glucose in the sample.

98. The method of claim 97, wherein said sample is a non-clinical sample such as food or beverage composition or a fluid obtained from a plant.

99. The method of claim 98, wherein said food or beverage composition comprises meat, canned food, dairy, nondairy, a fermented food, a fruit, a vegetable, a tuber, a starch, a grain, pasta, yogurt, soup, ice cream, a broth, a puree, a shake, a smoothie, a batter, a condiment, a sauce, a soft drink, a fountain beverage, or water, coffee, tea, milk, a dairy-based beverages, soy-based beverage, an almond-based beverage, vegetable juice, fruit juice, a fruit juice-flavored drink, an energy drink, or an alcoholic beverage.

100. A method for monitoring the level of glucose in a subject, comprising (a) administering a biosensor according to claim 83 or a device comprising a biosensor according to claim 83 to said subject, wherein after administration the biosensor is in contact with a biological fluid or surface of said subject, and (b) detecting (i) a signal produced by a reporter group of said biosensor continuously or repeatedly at intervals less than about 30 minutes apart, and/or (ii) whether a signal is produced by a reporter group of said biosensor continuously or repeatedly at intervals less than about 30 minutes apart.

101. The method of claim 100, wherein the biological sample comprises sweat, tear fluid, blood, serum, plasma, interstitial fluid, amniotic fluid, sputum, gastric lavage, skin oil, milk, fecal matter, emesis, bile, saliva, urine, mucous, semen, lymph, spinal fluid, synovial fluid, or a cell lysate.

102. A device comprising a biosensor according to claim 83.

103. The device of claim 102, wherein the biosensor is attached to a surface or matrix of the device, wherein the surface or matrix comprises a polymer.

104. The device of claim 103, wherein the polymer is cellulose.

105. The device of claim 102, wherein said device comprises an optode, a dermal patch, a contact lens, or a bead that is suitable for subcutaneous administration.
